(19) United States
(12) Patent Application Publication
(10) Pub. No.: US 2026/0078130 A1
(43) Pub. Date: Mar. 19, 2026

(54) NAMPT MODULATORS, PREPARATIONS, AND USES THEREOF

(71) Applicant: SIRONAX LTD., Grand Cayman (KY)

(72) Inventors: Xiaoqing WANG, Beijing (CN); Weijie HOU, Beijing (CN); Ming JIANG, Beijing (CN); Shaoqiang YANG, Beijing (CN); Tao SHENG, Beijing (CN); Guangxin LI, Beijing (CN); Jie LU, Beijing (CN); Yingtao LIU, Shanghai (CN); Yu CHEN, Shanghai (CN); Lianzhu LIU, Shanghai (CN); Yimin JIANG, Beijing (CN); Yanping XU, Beijing (CN); Zhiyuan ZHANG, Beijing (CN)

(21) Appl. No.: 19/109,179

(22) PCT Filed: Sep. 22, 2023
(86) PCT No.: PCT/CN2023/120632

§ 371 Date: Mar. 6, 2025

(30) Foreign Application Priority Data
Sep. 23, 2022 (WO) PCT/CN2022/120763
Feb. 13, 2023 (WO) PCT/CN2023/075667

(51) Int. Cl. C07D 487/10; A61K 31/4035; A61K 31/4155; A61K 31/416; A61K 31/4184; A61K 31/4192; A61K 31/422; A61K 31/423; A61K 31/4245; A61K 31/427; A61K 31/428; A61K 31/429; A61K 31/433; A61K 31/437; A61K 31/4439; A61K 31/444; A61K 31/454; A61K 31/4704; A61K 31/4725; A61K 31/4985; A61K 31/501; A61K 31/506; A61K 31/517; A61K 31/5377; A61K 31/538; C07B 59/00; C07D 209/46; C07D 209/48; C07D 215/227; C07D 217/24; C07D 231/56; C07D 265/36; C07D 401/06; C07D 401/12; C07D 401/14; C07D 403/04; C07D 403/06; C07D 403/12; C07D 403/14; C07D 405/14; C07D 413/06; C07D 413/14; C07D 417/06; C07D 417/14; C07D 471/04; C07D 487/04; C07D 491/107; C07D 498/04; C07D 513/04

(52) U.S. Cl. CPC C07D 487/10; A61K 31/4035; A61K 31/4155; A61K 31/416; A61K 31/4184; A61K 31/4192; A61K 31/422; A61K 31/423; A61K 31/4245; A61K 31/427; A61K 31/428; A61K 31/429; A61K 31/433; A61K 31/437; A61K 31/4439; A61K 31/444; A61K 31/454; A61K 31/4704; A61K 31/4725; A61K 31/4985; A61K 31/501; A61K 31/506; A61K 31/517; A61K 31/5377; A61K 31/538; C07B 59/002; C07D 209/46; C07D 209/48; C07D 215/227; C07D 217/24; C07D 231/56; C07D 265/36; C07D 401/06; C07D 401/12; C07D 401/14; C07D 403/04; C07D 403/06; C07D 403/12; C07D 403/14; C07D 405/14; C07D 413/06; C07D 413/14; C07D 417/06; C07D 417/14; C07D 471/04; C07D 487/04; C07D 491/107; C07D 498/04; C07D 513/04; C07B 2200/05

(57) ABSTRACT

This disclosure provides compounds of Formula 1 or 8, compositions comprising the same, and methods of using the same, including uses in modulating NAMPT and treating various diseases and conditions that are responsive to NAMPT activation.

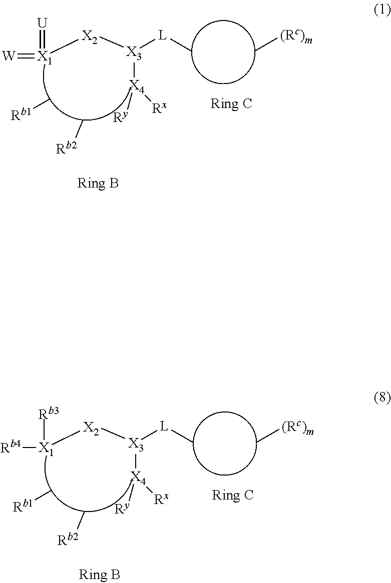

### RELATED APPLICATIONS

This application claims priority to International Application No. PCT/CN2022/120763, filed on Sep. 23, 2022, and International Application No. PCT/CN2023/075667, filed on Feb. 13, 2023. The contents of both applications are incorporated by reference in their entireties.

### FIELD OF THE DISCLOSURE

The present disclosure relates to compounds that modulate nicotinamide phosphoribosyltransferase (NAMPT), compositions comprising the compounds, methods of preparing the compounds, and methods of using the compounds to treat various diseases or conditions that benefit from NAMPT activation.

### BACKGROUND OF THE DISCLOSURE

Nicotinamide phosphoribosyltransferase (NAMPT), a dimeric type II phosphoribosyl transferase, is a key enzyme in the dominant nicotinamide adenine dinucleotide (NAD⁺) metabolism pathway in humans. Specifically, NAMPT catalyzes the rate limiting reaction in this pathway to convert nicotinamide and phosphoribosyl pyrophosphate to an intermediate nicotinamide mononucleotide (NMN), which is subsequently converted to NAD. NAD⁺, in turn, is important in serving as an electron-carrying coenzyme for oxidoreductases that play key roles in cellular energy generation pathways including the tricarboxylic acid cycle (TCA). NAD⁺ also serves as an ADP donator for other enzymes such as sirtuins (SIRTs) and poly(ADP-ribose) polymerases (PARPs) which have critical functions in energy homeostasis, cell signaling and division, and DNA repair. Because of the prominence of NAD⁺ to human biology, especially its function in connection with SIRTs and PARPs, deficiency in NAD⁺ has been shown to cause a wide spectrum of diseases such as cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

There is significant interest in NAD⁺ as a potential target for therapeutics and various strategies have been proposed to boost NAD⁺ levels in patients. One approach seeks to increase levels of NAD⁺ directly by supplementing its biosynthetic precursors including nicotinic acid (NA) and nicotinamide mononucleotide (NAM) and another approach seeks to do so by inhibiting NAD⁺ consumption, such as by prescribing PARP inhibitors. However, these efforts have not achieved desired results due to challenges in pharmacological efficacy and safety. For example, NA supplement could cause cutaneous flushing through GPR109A activation and PARP inhibitors could cause side effects as a result of underlying toxicity.

Recently, NAMPT activation has emerged as an alternative treatment approach that promises safe and effective elevation of NAD⁺ levels. A number of small-molecule activators were discovered that demonstrated non-toxicity and efficacy in boosting NAD⁺ levels. For example, a series of aminopropyl carbazole derivatives, named P7C3, have been identified that have been shown to significantly increase levels of NAD⁺ in human cell line. Additionally, other NAMPT activator candidates including a series of urea-containing small molecules SBI-797812 and DS68702229 have been identified, with varying degrees of efficacy and pharmacokinetic profiles.

Certain NAMPT activators are disclosed in WO 2020/010252A1, WO2017161261A1, WO2018132372 A1, WO2021159015, WO2021226276, and WO2022109311A1. Background information about NAMPT and certain NAMPT activators are provided by Colon, et al., Biochemical Pharmacology 198 (2022) 114946; Verdin, Science, VOL 350, ISSUE 6265, 1208; Wang et al., European Journal of Medicinal Chemistry 236 (2022) 114260; Wang et al., Cell, 158, 1324-1334; Gardell et al., NATURE COMMUNICATIONS (2019) 10:3241|https://doi.org/10.1038/s41467-019-11078-z; Akiu, et al., Chem. Pharm. Bull. 69, 1110-1122 (2021).

More research in this area is needed to identify new candidates for NAMPT activation. Described herein are small molecule activators of NAMPT and use of such activators for treating or preventing diseases or conditions responsive to increased NAMPT activity.

### SUMMARY OF THE DISCLOSURE

One aspect of this disclosure provides a compound selected from compounds of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, which can be employed in the treatment of various diseases or conditions, such as diseases or conditions that benefit from NAMPT activation. For example, disclosed herein is a compound of the following structural Formula 1:

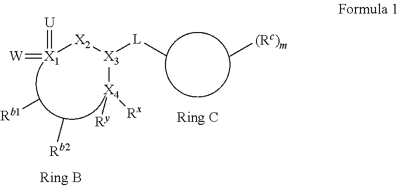

a tautomer thereof, a solvate or stereoisomer of the compound or the tautomer, or a pharmaceutically acceptable salt of the foregoing, wherein:
X₁ is C or S, provided that when X₁ is C, W is absent, and when X₁ is S, W is O or absent;
U is selected from O and S provided that when X₁ is S, U cannot be S;
X₂ is C, N, or absent; X₃ is C or N; X₄ is C, N, or absent;
Ring B is a 5- to 6-membered heterocyclic group, wherein Ring B comprises zero or one heteroatom at a position other than the positions of X₁, X₂, X₃, and X₄;
Ring C is phenyl, a 9- to 10-membered aryl, a 5- to 6-membered heteroaryl, a 9- to 10-membered heteroaryl, a 3- to 6-membered carbocyclyl, or a 4- to 12-membered heterocyclic group;
L is selected from

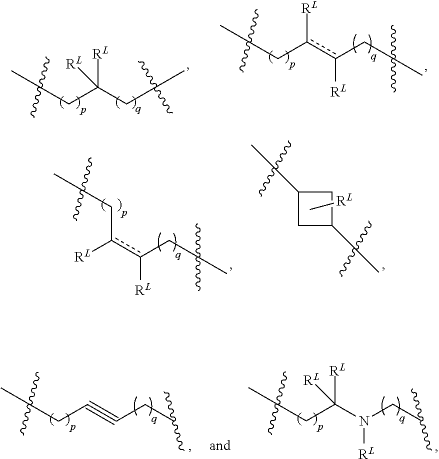

wherein R^{L}, for each occurrence, is independently selected from H, halogen, deuterium, and C₁ to C₃ alkyl optionally substituted with 1-3 groups selected from halogen;
R^{b1} and R^{b2} are attached to two adjacent positions in Ring B and R^{b1} and R^{b2} join to form Ring A, wherein Ring A is a phenyl or 5- to 6-membered heteroaryl group and wherein Ring A is substituted with z groups of R^{a};
R^{a}, for each occurrence, is independently selected from halogen, CN, C₁ to C₄ alkyl, —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3-to 7-membered carbocyclyl, and 3- to 7-membered heterocyclyl,
   wherein:
   the C₁ to C₄ alkyl of R^{a} is optionally substituted with 1-3 groups selected from —NR^{p}R^{q}, —C(═O)NR^{p}R^{q}, phenyl, 5- to 6-membered heteroaryl, 3- to 7-membered carbocyclyl, 3- to 7-membered heterocyclyl, halogen, and OR^{s},
   the 5- to 6-membered heteroaryl, the 3- to 7-membered carbocyclyl, or the 3- to 7-membered heterocyclyl of R^{a} is optionally substituted with 1-2 groups selected from C₁ to C₄ alkyl, —O(C₁ to C₄ alkyl), —N(C₁ to C₄ alkyl)₂, and —NH(C₁ to C₄ alkyl);
R^{x} is selected from H, ═O, CN, C₃ to Ce carbocyclyl, C₁ to C₄ alkyl,

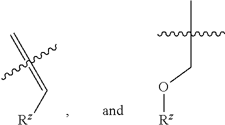

wherein:
   the C₁ to C₄ alkyl of R^{x} is optionally substituted with 1-3 groups selected from halogen, deuterium, CN, 5- to 6-membered carbocyclyl, 5- to 9-membered heterocyclyl, 6-membered aryl, 5- to 6-membered heteroaryl, and OH,
      wherein: the 6-membered aryl or the 5- to 6-membered heteroaryl of the C₁ to C₄ alkyl of R^{x} is optionally substituted with 1-3 groups selected from halogen, OR^{s}, CN, C(═O)NR^{p}R^{q}, NR^{p}R^{q}, C₁-C₆ alkyl (which is optionally substituted with 1-3 groups selected from halogen, O—R^{z}, OR^{s}, and 4- to 6-membered heterocyclyl optionally substituted with C₁ to C₂ alkyl), C₁-C₆ alkenyl, C₁-C₆ alkynyl, 3- to 6-membered carbocyclyl, and 4- to 7-membered heterocyclyl (which is optionally substituted with 1-3 groups selected from optionally substituted C₁ to C₄ alkyl, halogen, and OR^{s}), and
      wherein: the 5- to 9-membered heterocyclyl of the C₁ to C₄ alkyl of R^{x} is optionally substituted with 1-2 groups selected from C₁ to C₆ alkyl, ═O, and halogen,
   R^{z} is selected from H, 6-membered aryl, and 5- to 6-membered heteroaryl,
      wherein: the 6-membered aryl or 5- to 6-membered heteroaryl of R^{z} is optionally substituted with 1-2 groups selected from halogen;
R^{y} is selected from H, C₁ to C₂ alkyl, and absent;
R^{c}, for each occurrence, is independently selected from deuterium, halogen, C₁ to C₄ alkyl, ═O, ═S, OR^{s}, —NHC(═O)R^{s}, —NHC(═O)OR^{s}, NR^{p}R^{q}, —NHC(═O)NR^{p}R^{q}, CN, optionally substituted 5- to 6-membered heteroaryl, optionally substituted 5- to 6-membered heterocyclyl, and —C(═O)NR^{p}R^{q};
   wherein:
   the C₁ to C₄ alkyl of R is optionally substituted with 1-3 groups selected from halogen and OR^{s};
wherein:
   R^{p}, for each occurrence, is independently selected from H and C₁ to C₆ alkyl, R^{q}, for each occurrence, is independently selected from H, C₁ to C₆ alkyl, phenyl, 5- to 6-membered heteroaryl, 4- to 7-membered heterocyclyl, and CN, or
   R^{p} and R^{q} join to form a 3- to 6-membered carbocyclyl;
   R^{s}, for each occurrence, is independently selected from H, C₁ to C₆ alkyl (which is optionally substituted with 1-3 groups selected from halogen, deuterium, O(C₁ to C₄ alkyl), and NR^{p}R^{q}), phenyl, and 4- to 7-membered heterocyclyl;
   wherein:
   the 4- to 7-membered heterocyclyl of R^{q} and R^{s}, in each occurrence, is independently optionally substituted with 1-2 groups selected from C₁ to C₄ alkyl and —O(C₁ to C₄ alkyl); and
wherein:
m is an integer selected from 0, 1, 2, and 3;
p is an integer selected from 0, 1, 2, and 3;
q is an integer selected from 0, 1, 2, and 3;
z is an integer selected from 0, 1, 2, 3, and 4; and
the sum of p and q is an integer equal to or less than 5.

For another example, disclosed herein is a compound of the following structural Formula 8:

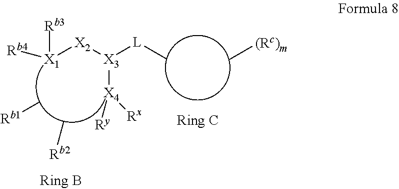

a tautomer thereof, a solvate or stereoisomer of the compound or the tautomer, or a pharmaceutically acceptable salt of the foregoing, wherein R^{b3} and R^{b4} are independently selected from H and C₁ to C₃ alkyl, or R^{b3} and R^{b4} join to form a 3- to 4-membered carbocyclyl, and wherein all other variables not specifically defined herein are defined in the preceding embodiment.

In one aspect of the disclosure, the compounds of the Formulae disclosed herein are selected from Compounds 1 to 645 shown in Table 1, a tautomer thereof, a solvate or stereoisomer of the compound or the tautomer, or a pharmaceutically acceptable salt of the foregoing.

In some embodiments, the disclosure provides pharmaceutical compositions comprising a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, and a pharmaceutically acceptable carrier. In some embodiments, the pharmaceutical compositions may comprise a compound selected from Compounds 1 to 645 shown in Table 1, a tautomer thereof, a solvate or stereoisomer of the compound or the tautomer, or a pharmaceutically acceptable salt of the foregoing, and a pharmaceutically acceptable carrier. These compositions may further comprise an additional active pharmaceutical agent.

Another aspect of the disclosure provides methods of treating a disease or condition, comprising administering to a subject in need thereof, a therapeutically effective amount of a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition is selected from cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

A further aspect of the disclosure provides methods of treating a disease or condition that benefit from NAMPT activation, comprising administering to a subject in need thereof, a therapeutically effective amount of a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administering to a subject in need thereof, a compound selected from Compounds 1 to 645 shown in Table 1, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In some embodiments, the methods of treatment comprise administration of an additional active pharmaceutical agent to the subject in need thereof, either in the same pharmaceutical composition as a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or in a separate composition. In some embodiments, the methods of treatment comprise administering a compound selected from Compounds 1 to 645 shown in Table 1, a tautomer thereof, a solvate or stereoisomer of the compound or the tautomer, or a pharmaceutically acceptable salt of the foregoing with an additional active pharmaceutical agent either in the same pharmaceutical composition or in a separate composition. When administered as a separate composition, the additional therapeutic agent may be administered prior to, at the same time as, or following administration of the compound, tautomer, solvate, stereoisomer, or a pharmaceutically acceptable salt disclosed herein.

Also disclosed herein are methods of modulating, e.g., activating, NAMPT in a subject in need thereof, comprising contacting the subject with a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1), a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of modulating, e.g., activating, NAMPT in a subject in need thereof comprise contacting the subject with a compound selected from Compounds 1 to 645 shown in Table 1, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

Also disclosed herein are methods of increasing NAD⁺ level in a subject in need thereof, comprising contacting the subject with a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt. In some embodiments, the methods of increasing NAD⁺ level mediated by NAMPT in a subject in need thereof comprise contacting the subject with a compound selected from Compounds 1 to 645 shown in Table 1, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “alkyl” refers to a hydrocarbon group selected from linear and branched saturated hydrocarbon groups, containing 1-20, e.g., 1-18, 1-12, 1-10, 1-8, 1-6, 1-4, or 1-3, carbon atoms. Examples of the alkyl group include methyl, ethyl, 1-propyl or n-propyl (“n-Pr”), 2-propyl or isopropyl (“i-Pr”), I-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of an alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl groups. Lower alkyl contains 1-8, preferably 1-6, more preferably 1-4 carbon atoms, and more preferably 1-3 carbon atoms.

The term “alkenyl” refers to a hydrocarbon group selected from linear and branched hydrocarbon groups, comprising at least one C═C double bond and 2-20, e.g., 2-18, 2-12, 2-10, 2-8, 2-6, or 2-4, carbon atoms. Examples of the alkenyl group include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups. Lower alkenyl contains 2-8, preferably 2-6, and more preferably 2-4 carbon atoms.

The term “alkynyl” refers to a hydrocarbon group selected from linear and branched hydrocarbon groups, comprising at least one C≡C triple bond and 2-20, e.g., 2-18, 2-12, 2-10, 2-8, 2-6, or 2-4, carbon atoms. Examples of the alkynyl group include ethynyl, 1-propynyl, 2-propynyl (propargyl), 1-butynyl, 2-butynyl, and 3-butynyl groups. Lower alkynyl contains 2-8, preferably 2-6, and more preferably 2-4 carbon atoms.

The term “heteroalkyl” refers to an alkyl group, as defined herein, in which one or more of the constituent carbon atoms have been replaced by a heteroatom, e.g., nitrogen, oxygen, or sulfur, e.g., CH₃CH₂OH, CH₃CH₂OC₂H₅, CH₃CH₂SH, CH₃CH₂SC₂H₅, CH₃CH₂NH₂, CH₃CH₂NHC₂H₅, etc. In some embodiments, in addition to the replacement of one or more of the constituent carbon atoms by nitrogen, oxygen, or sulfur, a heteroalkyl group is further optionally substituted as defined herein.

The term “cycloalkyl” refers to a hydrocarbon group selected from saturated and partially unsaturated cyclic hydrocarbon groups, e.g., monocyclic and polycyclic (e.g., bicyclic and tricyclic) groups. For example, the cycloalkyl group may be of 3-12, 3-10, 3-8, 3-6, 3-4, or 5-6 carbon atoms. Even further for example, the cycloalkyl group may be a monocyclic group of 3-12, 3-8, 3-6, 3-4, or 5-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6], and [6,6]ring systems, or as a bridged bicyclic ring selected from bicyclo[2.2.1]heptane, bicyclo[2.2.2]octane, and bicyclo[3.2.2]nonane. The ring may be saturated or have at least one double bond (i.e., partially unsaturated), but is not fully conjugated, and is not an aromatic ring, as “aromatic ring” is defined herein.

The term “heterocyclic” or “heterocycle” or “heterocyclyl” refers to a ring selected from 3- to 12-membered, e.g., 3- to 6-membered, 3- to 5-membered, 4- to 5-membered, or 5- to 6-membered, monocyclic, bicyclic, and tricyclic, saturated and partially unsaturated rings comprising at least one carbon atom in addition to 1, 2, 3, or 4 heteroatoms, selected from, e.g., oxygen, sulfur, nitrogen, and silicon. “Heterocycle” also refers to a 5- to 7-membered heterocyclic ring comprising at least one heteroatom selected from N, O, and S fused with 5-, 6-, and/or 7-membered cycloalkyl, carbocyclic aromatic, or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e., partially unsaturated). A heterocycle may be substituted with oxo. The point of the attachment may be carbon or heteroatom in the heterocyclic ring. A heterocycle is not a heteroaryl as defined herein.

Examples of heterocycles include, but are not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl, 1,4-diazepanyl, 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl, and 1, 1-dioxo-1-thiomorpholinyl.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6], and [6,6]ring systems as mentioned above; a fused bicyclic aryl ring such as 7- to 12-membered bicyclic aryl ring systems as mentioned above, a fused tricyclic aryl ring such as 10- to 15-membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The term “heteroatom” means one or more of oxygen, sulfur, nitrogen, phosphorus, and silicon, including, any oxidized form of nitrogen or sulfur; the quaternized form of any basic nitrogen or a substitutable nitrogen of a heterocyclic ring, for example N (as in 3,4-dihydro-2H-pyrrolyl), NH (as in pyrrolidinyl) or NR⁺ (wherein R is, e.g., an optionally substituted alkyl group) (as in N-substituted pyrrolidinyl).

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds. A double bond may be depicted as [symbol] (two solid lines). The depiction of [symbol] (a solid line and a dashed line), as used herein, denotes a bond that may be a double bond or a single bond.

The term “alkoxy” as used herein, refers to an alkyl group, as defined above, wherein one carbon of the alkyl group is replaced by an oxygen atom, provided that the oxygen atom is linked between two carbon atoms.

The term “halogen” includes F, Cl, Br, and I, i.e., fluoro, chloro, bromo, and iodo, respectively.

As used herein, a “CN,” “cyano,” or “nitrile” group refers to —C≡N.

As used herein, an “aromatic ring” refers to a carbocyclic or heterocyclic ring that contains conjugated, planar ring systems with delocalized pi electron orbitals comprised of [4n+2]p orbital electrons, wherein n is an integer of 0 to 6. A “non-aromatic” ring refers to a carbocyclic or heterocyclic that does not meet the requirements set forth above for an aromatic ring, and can be either completely or partially saturated. Non-limiting examples of aromatic rings include aryl and heteroaryl rings that are further defined as follows. An “aromatic ring” may be depicted as a cycle with conjugated double bonds, such as

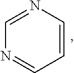

or as a cycle with an inside circle, such as

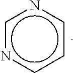

The term “aryl” herein refers to a group selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group selected from: 5- to 7-membered, e.g., 5- to 6-membered, aromatic, monocyclic rings comprising 1, 2, 3, or 4 heteroatoms selected from N, O, and S, with the remaining ring atoms being carbon; 8- to 12-membered bicyclic rings comprising 1, 2, 3, or 4 heteroatoms, selected from N, O, and S, with the remaining ring atoms being carbon and wherein at least one ring is aromatic and at least one heteroatom is present in the aromatic ring; and 11- to 14-membered tricyclic rings comprising 1, 2, 3, or 4 heteroatoms, selected from N, O, and S, with the remaining ring atoms being carbon and wherein at least one ring is aromatic and at least one heteroatom is present in an aromatic ring.

For example, the heteroaryl group may be a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinolinyl.

The term “acyl” refers to a substituent group where a point of attachment in the substituent group is a carbonyl. Exemplary acyl groups include, but are not limited to, —C(═O)R^{s}, —C(═O)NR′R″, or —C(═O)OR^{s}, wherein R′ and R″ are independently selected from hydrogen, alkyl, alkenyl, alkynyl, heteroalkyl, heteroalkenyl, heteroalkynyl, cycloalkyl, aryl, heterocyclyl, or heteroaryl, any of which may be further substituted by one or more substituents.

Some of the compounds may exist with different points of attachment of hydrogen, referred to as “tautomers.” For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereoisomers using optically active resolving agents. Racemic mixtures of chiral compounds of the disclosure can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereoisomers, and conversion to the pure stereoisomers, and (3) separation of the substantially pure or enriched stereoisomers directly under chiral conditions.

The term “substantially pure” in the context of stereoisomers means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

Unless otherwise indicated, structures depicted herein are meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the compounds disclosed herein are within the scope of the disclosure. Unless otherwise stated, all tautomeric forms of the compounds of the disclosure are within the scope of the disclosure.

The disclosure provides pharmaceutically acceptable salts of the disclosed compounds, tautomers, solvates, and stereoisomers. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, magnesium, aluminum, lithium, and ammonium. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, benzenesulfonic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate (i.e., caprate), caprylate, acrylate, formate, isobutyrate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, P-hydroxybutyrate, glycolate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium salts. Further non-limiting examples of pharmaceutically acceptable salts include salts of ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

If a compound is obtained as an acid addition salt, the free base can be obtained by basifying a solution of the acid addition salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The compounds, tautomers, solvates, stereoisomers, and pharmaceutically acceptable salts of the disclosure may also contain unnatural proportions of atomic isotopes at one or more of the atoms that constitute such compounds. For example, —CD₃, —CD₂—, —CDH—, —CD₂H, or —CDH₂ contains one or more deuteriums in place of hydrogen. For example, the compounds may be radiolabeled with radioactive isotopes, such as for example tritium (³H), iodine-125 (¹²⁵I), or carbon-14 (¹⁴C). All isotopic variations of the compounds of the disclosure, whether radioactive or not, are intended to be encompassed within the scope of the disclosure.

As used herein, “optionally substituted” is interchangeable with the phrase “substituted or unsubstituted.” In general, the term “substituted,” refers to the replacement of a hydrogen radical in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. When a substituent is attached to a ring structure without a specified position such as in

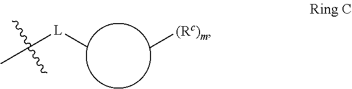

the substituent, e.g., R^{c}, may be attached to any chemically feasible position of Ring C regardless of whether Ring C is single cyclic or multi-cyclic structure, when m is a positive integer. For example, unless otherwise specified, R^{c}, as shown in

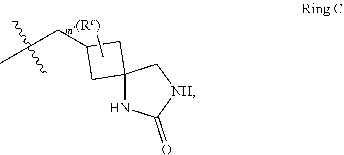

may be attached to any chemically feasible position of the 4-membered cyclic structure of Ring C or any chemically feasible position (e.g., C or N) of the 5-membered cyclic structure of Ring C, when m′ is a positive integer. For another example, R^{c}, as shown in

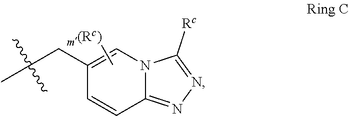

Ring C may be attached to any chemically feasible position of the 6-membered cyclic structure of Ring C (when m′ is a positive integer) and to a fixed position of the 5-membered cyclic structure of Ring C as specified.

Combinations of chemical components, e.g., substituents, ring structures, linkers, and/or heteroatoms, envisioned by this disclosure are those that result in the formation of stable or chemically feasible compounds.

In some embodiments, substituents (such as in situations where a group is optionally substituted with one or more substituents, e.g., optionally substituted phenyl) are independently selected from optionally substituted heteroatom and optionally substituted, optionally hetero-, optionally cyclic C₁-C₁₈ hydrocarbyl, particularly wherein the optionally substituted, optionally hetero-, optionally cyclic C₁-C₁₈ hydrocarbyl is optionally-substituted, optionally hetero-, optionally cyclic alkyl, alkenyl or alkynyl, or optionally-substituted, optionally hetero-, aryl; and/or the optionally substituted heteroatom is halogen, optionally substituted hydroxyl (such as alkoxy, aryloxy), optionally substituted acyl (such as formyl, alkanoyl, carbamoyl, carboxyl, amido), optionally substituted amino (such as amino, alkylamino, dialkylamino, amido, sulfamidyl), optionally substituted thiol (such as mercapto, alkylthiol, aryl thiol), optionally substituted sulfinyl or sulfonyl (such as alkylsulfinyl, arylsulfinyl, alkyl sulfonyl, arylsulfonyl), nitro, or cyano.

In some embodiments, substituents are independently selected from: halogen, —R′, —OR′, ═O, ═NR′, ═N—OR′, —NR′R″, —SR′, —SiR′R″R′, —OC(═O)R′, —C(═O)R′, —CO₂R′, —C(═O)NR′R″, —OC(═O)NR′R″, —NR″C(═O)R′, —NR′—C(═O)NR″R″, —NR′—SO₂NR″R′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R′, —CN, —NO₂, —N₃, —CH(Ph)₂, perfluoro(C₁-C₄)alkoxy, and perfluoro(C₁-C₄)alkyl, in a number ranging from zero to three, with those groups having zero, one, or two substituents being particularly preferred. R′, R″, and R′″ each independently refer to hydrogen, unsubstituted C₁-C₈ alkyl and heteroalkyl, C₁-C₈ alkyl and heteroalkyl substituted with one to three halogens, unsubstituted aryl, aryl substituted with one to three halogens, unsubstituted alkyl, alkoxy, or thioalkoxy groups, or aryl-(C₁-C₄) alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl. When the aryl group is 1,2,3,4-tetrahydronaphthalenyl, it may be substituted with a substituted or unsubstituted C₃-C₇ spirocycloalkyl group. The C₃-C₇ spirocycloalkyl group may be substituted in the same manner as defined herein for “cycloalkyl.”

In some embodiments, substituents are selected from: halogen, —R′, —OR′, ═O, —NR′R″, —SR′, —SiR′R″R′″, —OC(═O)R′, —C(═O)R′, —CO₂R′, —C(═O)NR′R″, —OC(═O)NR′R″, —NR″C(═O)R′, —NR″CO₂R′, —NR′—SO₂NR″R′″, —S(═O)R′, —SO₂R′, —SO₂NR′R″, —NR″SO₂R′, —CN, —NO₂, perfluoro C₁-C₄ alkoxy and perfluoro C₁-C₄ alkyl, where R′ and R″ are as defined above.

In some embodiments, substituents are independently selected from substituted or unsubstituted heteroatom, substituted or unsubstituted, 0-3 heteroatom-containing C₁-C₆ alkyl (e.g., C₁-C₃ alkyl or C₁-C₂ alkyl), substituted or unsubstituted, 0-3 heteroatom-containing C₂-C₆ alkenyl (e.g., C₂-C₄ alkenyl), substituted or unsubstituted, 0-3 heteroatom-containing C₂-C₆ alkynyl (e.g., C₂-C₄ alkynyl), or substituted or unsubstituted, 0-3 heteroatom-containing C₅-C₁₄ aryl (e.g., C₅-C₆ aryl), wherein each heteroatom is independently oxygen, phosphorus, sulfur, or nitrogen.

In some embodiments, substituents are independently selected from aldehyde, aldimine, alkanoyloxy, alkoxy, alkoxycarbonyl, alkyloxy, alkyl, alkenyl, alkynyl, amine, azo, halogen, carbamoyl, carbonyl, carboxamido, carboxyl, cyanyl, ester, haloformyl, hydroperoxyl, hydroxyl, imine, isocyanide, isocyante, N-tert-butoxycarbonyl, nitrate, nitrile, nitrite, nitro, nitroso, phosphate, phosphono, sulfide, sulfonyl, sulfo, sulfhydryl, thiol, thiocyanyl, trifluoromethyl, and trifluromethyl ether (OCF₃) groups.

Preferred substituents are disclosed herein and exemplified in the tables, structures, examples, and claims, and may be applied across different compounds of this disclosure. For example, substituents of a given compound may be combinatorically used with other compounds.

It may be advantageous to separate reaction products from one another and/or from starting materials. The desired products of each step or series of steps are separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example, reverse-phase and normal phase; size exclusion; ion exchange; high, medium, and low pressure liquid chromatography methods and apparatus; small scale analytical; simulated moving bed (“SMB”) and preparative thin or thick layer chromatography, as well as techniques of small scale thin layer and flash chromatography. One skilled in the art may apply such techniques to achieve a desired separation.

Non-limiting examples of suitable solvents that may be used in this disclosure include water, methanol (MeOH), ethanol (EtOH), dichloromethane or methylene chloride (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH), and sodium methoxide (NaOMe; NaOCH₃).

The term “subject” refers to an animal including a human.

The term “therapeutically effective amount” refers to the amount of a compound that produces a desired effect for which it is administered (e.g., improvement in a disease or condition, lessening the severity of a disease or condition, and/or reducing progression of a disease or condition, e.g., a disease or condition that can benefit from NAMPT activation. The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease or condition or a symptom thereof, lower risk of a disease or condition, e.g., a disease or condition that can benefit from NAMPT activation. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages, for example, a range of ±10% with respect to a specific point value) that is recognized by one of ordinary skill in the art.

### II. Compounds and Compositions

In a 1^{st} embodiment, a compound of this disclosure is a compound of the following structural Formula 1:

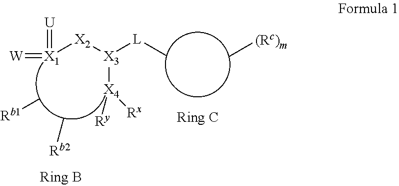

a tautomer thereof, a solvate or stereoisomer of the compound or the tautomer, or a pharmaceutically acceptable salt of the foregoing, wherein:
X₁ is C or S, provided that when X₁ is C, W is absent, and when X₁ is S, W is 0 or absent;
U is selected from O and S provided that when X₁ is S, U cannot be S;
X₂ is C, N, or absent; X₃ is C or N; X₄ is C, N, or absent;
Ring B is a 5- to 6-membered heterocyclic group, wherein Ring B comprises zero or one heteroatom at a position other than the positions of X₁, X₂, X₃, and X₄;
Ring C is phenyl, a 9- to 10-membered aryl, a 5- to 6-membered heteroaryl, a 9- to 10-membered heteroaryl, a 3- to 6-membered carbocyclyl (e.g., 3-, 4-, 5-, or 6-membered carbocyclyl group), or a 4- to 12-membered heterocyclic group (e.g., 4-, 5-, 6-, 7-, 8-, 9-, 10-, 11-, or 12-membered heterocyclic group);
L is selected from

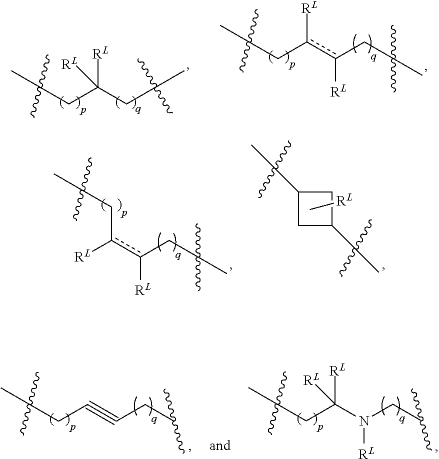

wherein R^{L}, for each occurrence, is independently selected from H, deuterium, halogen (e.g., F, Cl, or Br), and C₁ to C₃ alkyl optionally substituted with 1-3 groups selected from halogen (e.g., C₁ alkyl, C₂ alkyl, C₃ alkyl, or CF₃);
R^{b}i and R¹² are attached to two adjacent positions in Ring B and R^{b1} and R^{b2} join to form Ring A, wherein Ring A is a phenyl or 5- to 6-membered heteroaryl group and wherein Ring A is substituted with z groups of R^{a};
R^{a}, for each occurrence, is independently selected from halogen (e.g., F, Cl, or Br), CN, C₁ to C₄ alkyl (e.g., Cl, C₂, C₃, or C₄ alkyl), —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3- to 7-membered carbocyclyl (e.g., 3-, 4-, 5-, 6-, or 7-membered carbocyclyl), and 3- to 7-membered heterocyclyl (e.g., 3-, 4-, 5-, 6-, or 7-membered heterocyclyl),
   wherein:
   the C₁ to C₄ alkyl of R is optionally substituted with 1-3 groups selected from —NR^{p}R^{q}, —C(═O)NR^{p}R^{q}, phenyl, 5- to 6-membered heteroaryl, 3- to 7-membered carbocyclyl, 3- to 7-membered heterocyclyl, halogen, and OR^{s},
   the 5- to 6-membered heteroaryl, the 3- to 7-membered carbocyclyl, or the 3- to 7-membered heterocyclyl of R is optionally substituted with 1-2 groups selected from C₁ to C₄ alkyl, —O(C₁ to C₄ alkyl), —N(C₁ to C₄ alkyl)₂, and —NH(C₁ to C₄ alkyl);
R^{x} is selected from H, ═O, CN, C₃ to C₁ carbocyclyl, C₁ to C₄ alkyl,

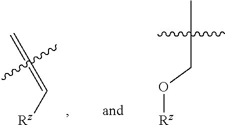

wherein:
   the C₁ to C₄ alkyl of R^{x} is optionally substituted with 1-3 groups selected from halogen, deuterium, CN, 5- to 6-membered carbocyclyl, 5- to 9-membered heterocyclyl (e.g., 5-, 6-, 7-, 8-, or 9-membered heterocyclyl), 6-membered aryl, 5- to 6-membered heteroaryl, and OH,
      wherein: the 6-membered aryl or the 5- to 6-membered heteroaryl of the C₁ to C₄ alkyl of R^{x} is optionally substituted with 1-3 groups selected from halogen, OR^{s}, CN, C(═O)NR^{p}R^{q}, NR^{p}R^{q}, C₁-C₆ alkyl (which is optionally substituted with 1-3 groups selected from halogen, O—R^{z}, OR^{s}, and 4- to 6-membered heterocyclyl optionally substituted with C₁ to C₂ alkyl), C₁-C₆ alkenyl (e.g., C₁, C₂, C₃, C₄, C₅, or C₆ alkenyl), C₁-C₆ alkynyl (e.g., C₁, C₂, C₃, C₄, C₅, or C₆ alkynyl), 3-to 6-membered carbocyclyl (e.g., 3-, 4-, 5-, or 6-membered carbocyclyl), and 4-to 7-membered heterocyclyl (e.g., 4-, 5-, 6-, or 7-membered heterocyclyl) (which is optionally substituted with 1-3 groups selected from optionally substituted C₁ to C₄ alkyl, halogen, and OR^{s}), and
      wherein: the 5- to 9-membered heterocyclyl of the C₁ to C₄ alkyl of R^{x} is optionally substituted with 1-2 groups selected from C₁ to C₆ alkyl (e.g., C₁, C₂, C₃, C₄, C₅, or C₆ alkyl), ═O, and halogen,
   R^{z} is selected from H, 6-membered aryl, and 5- to 6-membered heteroaryl,
      wherein: the 6-membered aryl or 5- to 6-membered heteroaryl of R^{z} is optionally substituted with 1-2 groups selected from halogen;
R^{y} is selected from H, C₁ to C₂ alkyl, and absent;
R^{c}, for each occurrence, is independently selected from deuterium, halogen, C₁ to C₄ alkyl, ═O, ═S, OR^{s}, —NHC(═O)R^{s}, —NHC(═O)OR^{s}, NR^{p}R^{q}, —NHC(═O)NR^{p}R^{q}, CN, optionally substituted 5- to 6-membered heteroaryl, optionally substituted 5- to 6-membered heterocyclyl, and —C(═O)NR^{p}R^{q},
   wherein:
   the C₁ to C₄ alkyl of R′ is optionally substituted with 1-3 groups selected from halogen and OR^{s};
wherein:
   R^{p}, for each occurrence, is independently selected from H and C₁ to C₆ alkyl, R^{q}, for each occurrence, is independently selected from H, C₁ to C₆ alkyl, phenyl, 5- to 6-membered heteroaryl, 4- to 7-membered heterocyclyl, and CN or
   R^{p} and R^{q} join to form a 3- to 6-membered carbocyclyl;
   R^{s}, for each occurrence, is independently selected from H, C₁ to C₆ alkyl (which is optionally substituted with 1-3 groups selected from halogen, deuterium, O(C₁ to C₄ alkyl), and NR^{p}R^{q}), phenyl, and 4- to 7-membered heterocyclyl;
   wherein:
   the 4- to 7-membered heterocyclyl of R^{q} and R^{s}, in each occurrence, is independently optionally substituted with 1-2 groups selected from C₁ to C₄ alkyl and —O(C₁ to C₄ alkyl); and
wherein:
m is an integer selected from 0, 1, 2, and 3;
p is an integer selected from 0, 1, 2, and 3;
q is an integer selected from 0, 1, 2, and 3;
z is an integer selected from 0, 1, 2, 3, and 4; and
the sum of p and q is an integer equal to or less than 5 (e.g., the sum of p and q is 0, 1, 2, 3, or 4).

Combinations of substituents as disclosed herein are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

In a 2^{nd} embodiment, a compound of the disclosure is a compound of the following structural Formula 2a or Formula 2a′:

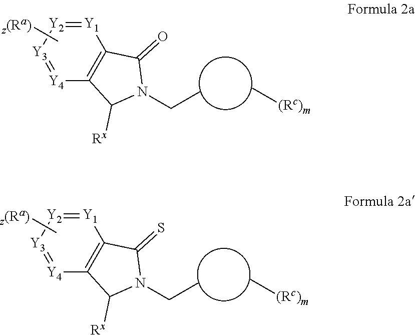

a tautomer thereof, a solvate or stereoisomer of the compound or the tautomer, or a pharmaceutically acceptable salt of the foregoing, wherein Y₁, Y₂, Y₃, and Y₄ are independently selected from C and N; and all other variables not specifically defined herein are as defined in the preceding embodiment. For example, one of Y₁, Y₂, Y₃, and Y₄ is N and the rest of them are C. For another example, two of Y₁, Y₂, Y₃, and Y₄ are N and the rest of them are C. For another example, three of Y₁, Y₂, Y₃, and Y₄ are N and the other one is C. For another example, all four of Y₁, Y₂, Y₃, and Y₄ are N. For another example, all four of Y₁, Y₂, Y₃, and Y₄ are C.

In a 3^{rd} embodiment, a compound of the disclosure is a compound of the following structural Formula 2b:

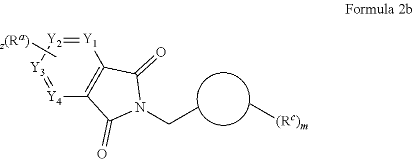

a tautomer thereof, a solvate or stereoisomer of the compound or the tautomer, or a pharmaceutically acceptable salt of the foregoing, wherein Y₁, Y₂, Y₃, and Y₄ are independently selected from C and N; and all other variables not specifically defined herein are as defined in any one of the suitable preceding embodiments. For example, one of Y₁, Y₂, Y₃, and Y₄ is N and the rest of them are C. For another example, two of Y₁, Y₂, Y₃, and Y₄ are N and the rest of them are C. For another example, three of Y₁, Y₂, Y₃, and Y₄ are N and the other one is C. For another example, all four of Y₁, Y₂, Y₃, and Y₄ are N. For another example, all four of Y₁, Y₂, Y₃, and Y₄ are C.

In a 4^{th} embodiment, a compound of the disclosure is a compound of the following structural Formula 2c, 2c-1, or 2c-2:

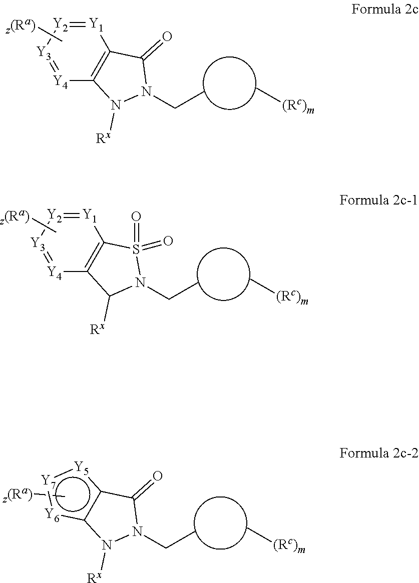

a tautomer thereof, a solvate or stereoisomer of the compound or the tautomer, or a pharmaceutically acceptable salt of the foregoing, wherein Y₁, Y₂, Y₃, and Y₄ are independently selected from C and N, and Y₅, Y₆ and Y₇ are independently selected from N, S, O and C; and all other variables not specifically defined herein are as defined in any one of the suitable preceding embodiments. For example, one of Y), Y₂, Y₃, and Y₄ is N and the rest of them are C. For another example, two of Y₁, Y₂, Y₃, and Y₄ are N and the rest of them are C. For another example, three of Y, Y₂, Y₃, and Y₄ are N and the other one is C. For another example, all four of Y₁, Y₂, Y₃, and Y₄ are N. For another example, all four of Y₁, Y₂, Y₃, and Y₄ are C.

In a 5^{th} embodiment, a compound of the disclosure is a compound of the following structural Formulae 2d to 2i:

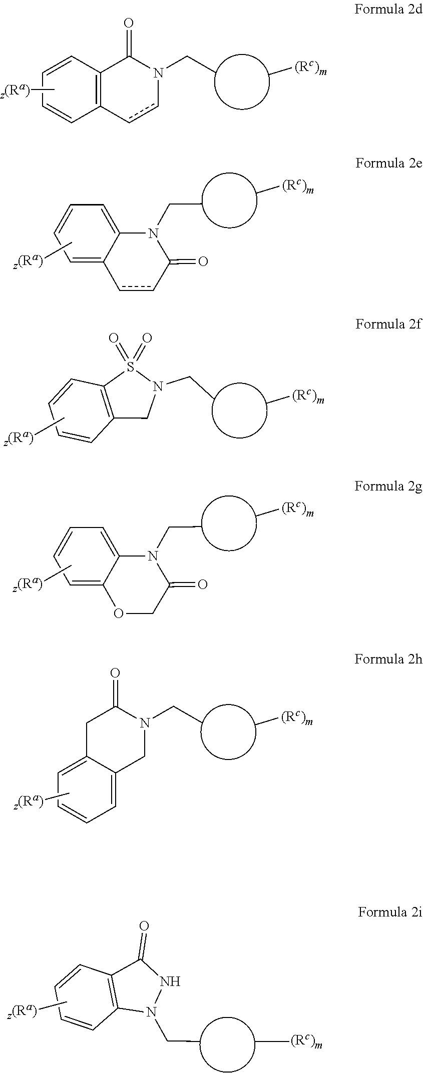

a tautomer thereof, a solvate or stereoisomer of the compound or the tautomer, or a pharmaceutically acceptable salt of the foregoing; and all other variables not specifically defined herein are as defined in any one of the suitable preceding embodiments.

In a 6^{th} embodiment, a compound of the disclosure is a compound of the following structural Formulae 3a and 3a-1 to 3a-16:

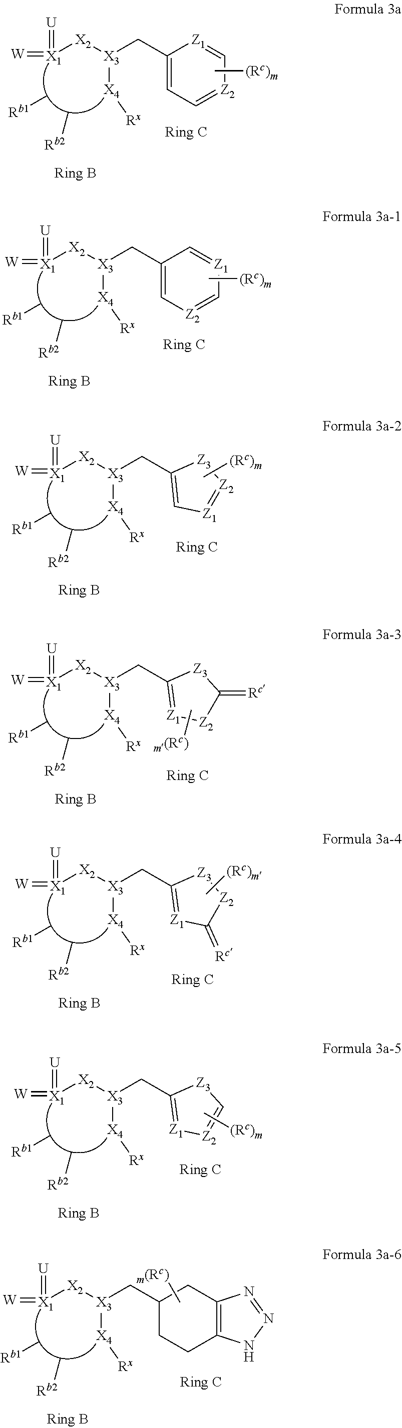

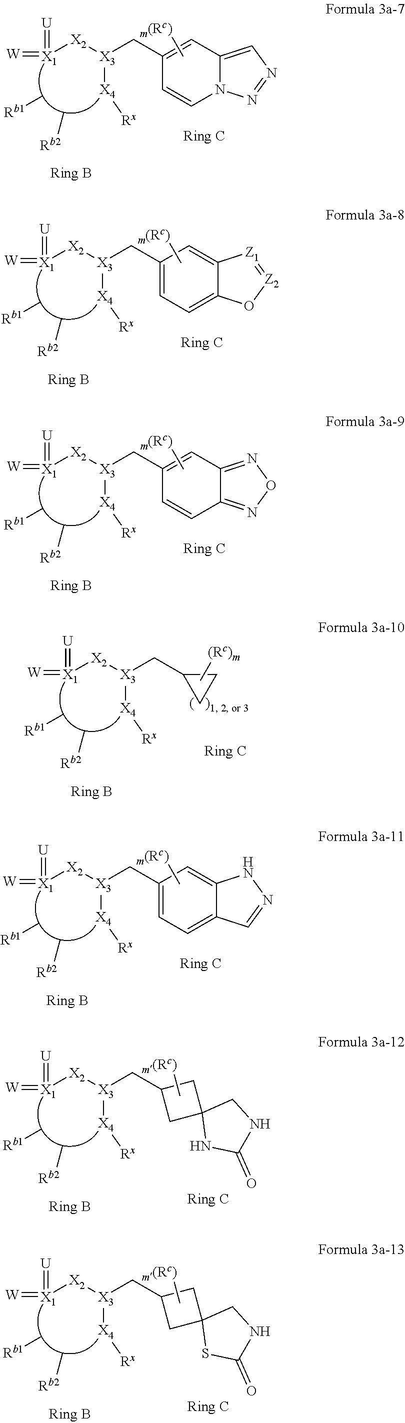

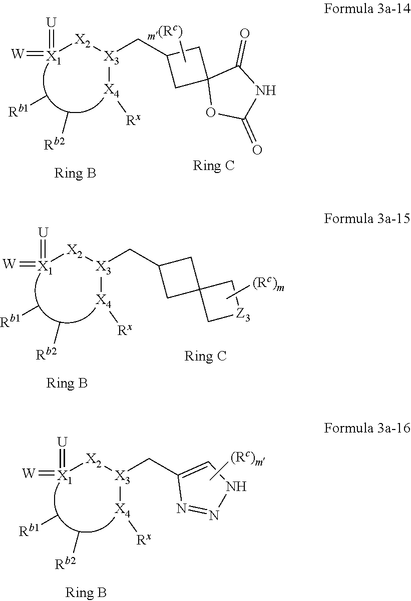

a tautomer thereof, a solvate or stereoisomer of the compound or the tautomer, or a pharmaceutically acceptable salt of the foregoing, wherein Z₁ and Z₂ are independently selected from C and N, Z₃ is selected from C, N, S, and O, R^{c′} is 0 or S, and m′, for each occurrence, is independently selected from 0, 1, and 2; and all other variables not specifically defined herein are as defined in any one of the suitable preceding embodiments. For example, one of Zi and Z₂ is N and the other one is C. For another example, both Z₁ and Z₂ are N. For another example, both Zi and Z₂ are C.

In a 7^{th} embodiment, a compound of the disclosure is a compound of the following structural Formulae 3b-3c:

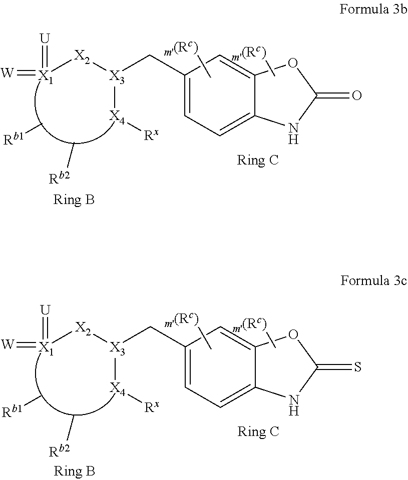

a tautomer thereof, a solvate or stereoisomer of the compound or the tautomer, or a pharmaceutically acceptable salt of the foregoing, wherein m′ is selected from 0 and 1; and all other variables not specifically defined herein are as defined in any one of the suitable preceding embodiments.

In a 8^{th} embodiment, a compound of the disclosure is a compound of the following structural Formulae 3d-3e:

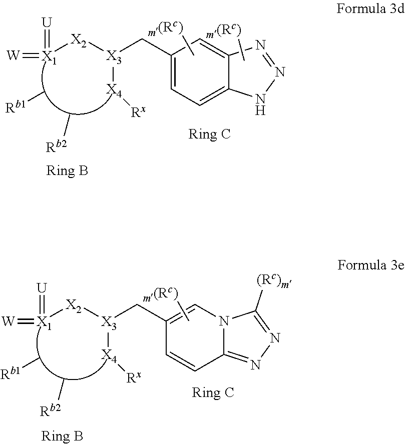

a tautomer thereof, a solvate or stereoisomer of the compound or the tautomer, or a pharmaceutically acceptable salt of the foregoing, wherein m′, for each occurrence, is independently selected from 0 and 1; and all other variables not specifically defined herein are as defined in any one of the suitable preceding embodiments.

In a 9^{th} embodiment, a compound of the disclosure is a compound of the following structural Formulae 3f-3h:

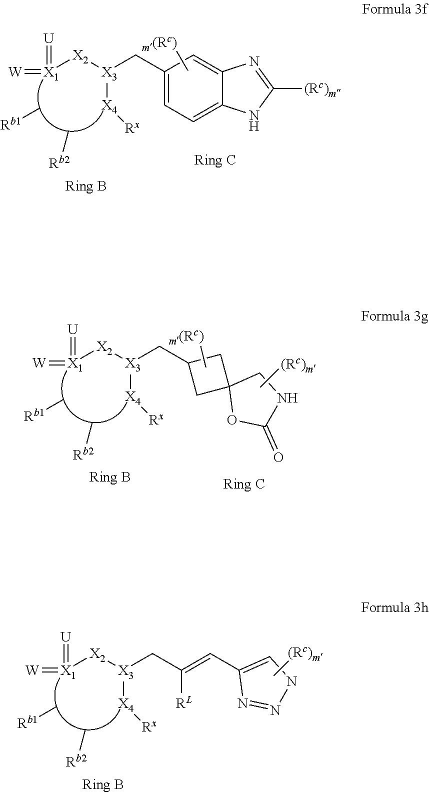

a tautomer thereof, a solvate or stereoisomer of the compound or the tautomer, or a pharmaceutically acceptable salt of the foregoing, wherein m′, for each occurrence, is independently selected from 0, 1, and 2, m″, for each occurrence, is independently selected from 0 and 1, R^{L} is selected from H and F; and all other variables not specifically defined herein are as defined in any one of the suitable preceding embodiments.

In a 10^{th} embodiment, a compound of the disclosure is a compound of the following structural Formulae 4a-4h:

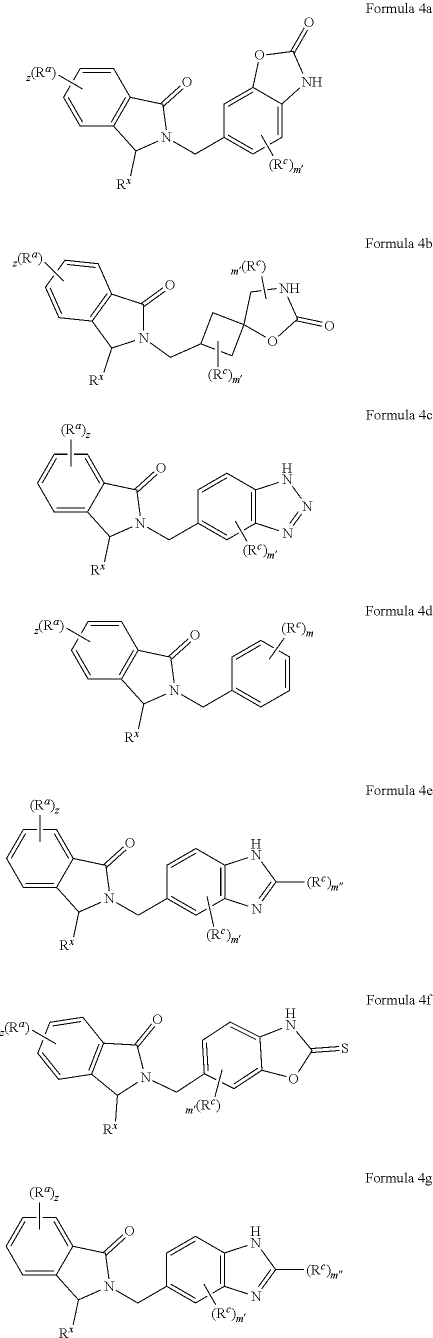

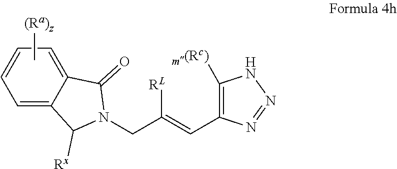

a tautomer thereof, a solvate or stereoisomer of the compound or the tautomer, or a pharmaceutically acceptable salt of the foregoing, wherein m′, for each occurrence, is independently selected from 0,1, and 2, m″, for each occurrence, is independently selected from 0 and 1, R^{L} is selected from H and F; and all other variables not specifically defined herein are as defined in any one of the suitable preceding embodiments.

In a 11^{th} embodiment, a compound of the disclosure is a compound of the following structural Formulae 5a-5e:

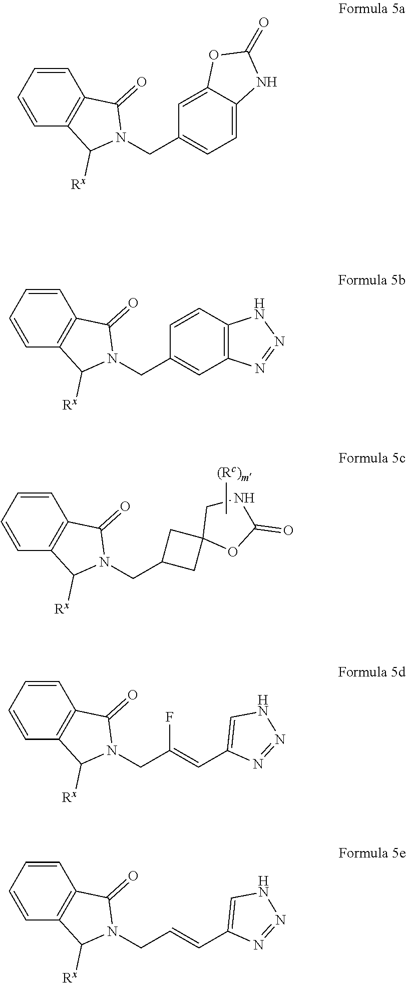

a tautomer thereof, a solvate or stereoisomer of the compound or the tautomer, or a pharmaceutically acceptable salt of the foregoing, R^{c} is deuterium, and m′ is selected from 0, 1, and 2; and all other variables not specifically defined herein are as defined in any one of the suitable preceding embodiments.

In a 12^{th} embodiment, a compound of the disclosure is a compound of the following structural Formulae 6a-6d:

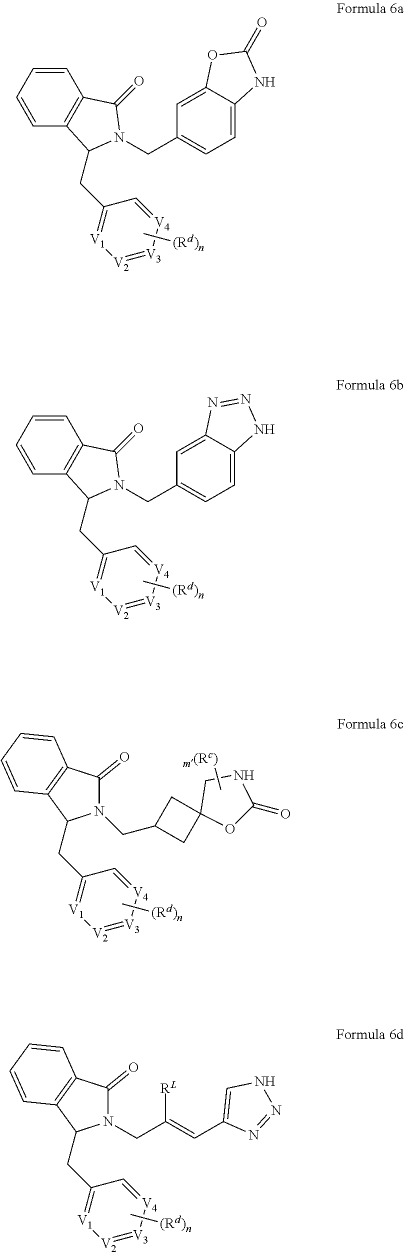

a tautomer thereof, a solvate or stereoisomer of the compound or the tautomer, or a pharmaceutically acceptable salt of the foregoing, wherein V₁, V₂, V₃, and V₄ are independently selected from C and N, R^{L} is selected from H and F, R^{c} is deuterium, R^{d}, for each occurrence, is independently selected from halogen, CN, OCH₃, C₁ to C₄ alkyl, C₁-C₄ alkenyl, C₁-C₄ alkynyl, 3- to 6-membered carbocyclyl, and 3- to 6-membered heterocyclyl, m′ is selected from 0, 1, and 2, and n is selected from 0, 1, 2, and 3; and all other variables not specifically defined herein are as defined in any one of the suitable preceding embodiments. For example, one of V₁, V₂, V₃, and V₄ is N and the rest of them are C. For another example, two of V₁, V₂, V₃, and V₄ are N and the rest of them are C. For another example, three of V₁, V₂, V₃, and V₄ are N and the other one is C. For another example, all four of V₁, V₂, V₃, and V₄ are N. For another example, all four of V₁, V₂, V₃, and V₄ are C.

In a 13^{th} embodiment, a compound of the disclosure is a compound of the following structural Formulae 6e-6h:

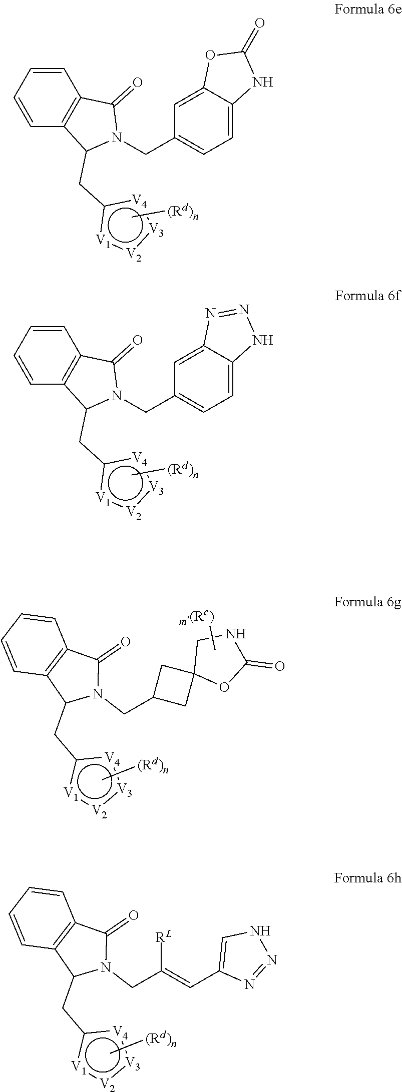

a tautomer thereof, a solvate or stereoisomer of the compound or the tautomer, or a pharmaceutically acceptable salt of the foregoing, wherein V₁, V₂, V₃, and V₄ are independently selected from C, O, S, and N, R^{L} is selected from H and F, R^{c} is deuterium, R^{d}, for each occurrence is independently selected from halogen, CN, OCH₃, C₁ to C₄ alkyl, C₁-C₄ alkenyl, C₁-C₄ alkynyl, 3- to 6-membered carbocyclyl, and 3- to 6-membered heterocyclyl, m′ is selected from 0, 1, and 2, and n, for each occurrence, is independently selected from 0, 1, 2 and 3; and all other variables not specifically defined herein are as defined in any one of the suitable preceding embodiments. For example, one of V₁, V₂, V₃, and V₄ is N and the rest of them are C. For another example, two of V₁, V₂, V₃, and V₄ are N and the rest of them are C. For another example, three of V₁, V₂, V₃, and V₄ are N and the other one is C. For another example, all four of V₁, V₂, V₃, and V₄ are N. For another example, one of V₁, V₂, V₃, and V₄ is 0, another one of V₁, V₂, V₃, and V₄ is N, and the rest of them are C. For another example, one of V₁, V₂, V₃, and V₄ is S, another one of V₁, V₂, V₃, and V₄ is N, and the rest of them are C. For another example, one of V₁, V₂, V₃, and V₄ is 0, another one of V₁, V₂, V₃, and V₄ is C, and the rest of them are N. For another example, one of V₁, V₂, V₃, and V₄ is S, another one of V₁, V₂, V₃, and V₄ is C, and the rest of them are N.

In a 14^{th} embodiment, in a compound, tautomer, a solvate or stereoisomer of the compound or the tautomer, or pharmaceutically acceptable salt of this disclosure, Ring A is selected from phenyl, pyridyl, and 5-membered heteroaryl containing 1 to 2 heteroatoms selected from N, S, and O; and all other variables not specifically defined herein are as defined in any one of the suitable preceding embodiments.

In a 15^{th} embodiment, in a compound, tautomer, a solvate or stereoisomer of the compound or the tautomer, or pharmaceutically acceptable salt of this disclosure, X₁ is C, X₂ is absent, and X₃ is C or N; and all other variables not specifically defined herein are as defined in any one of the suitable preceding embodiments. In one embodiment, X₄ is absent. In another embodiment, X₄ is present.

In a 16^{th} embodiment, in a compound, tautomer, a solvate or stereoisomer of the compound or the tautomer, or pharmaceutically acceptable salt of this disclosure, Ring C is selected from:

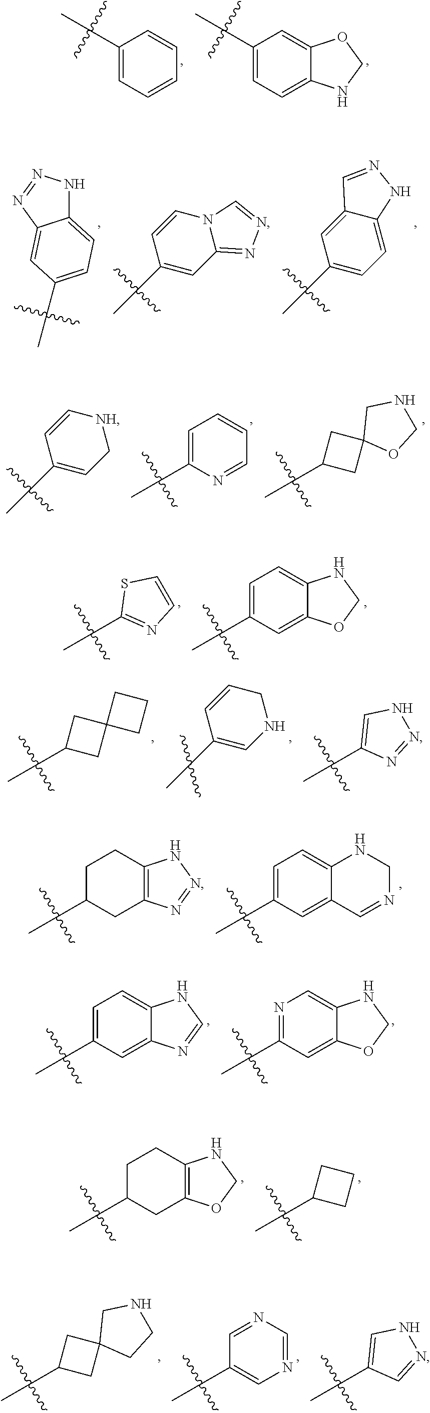

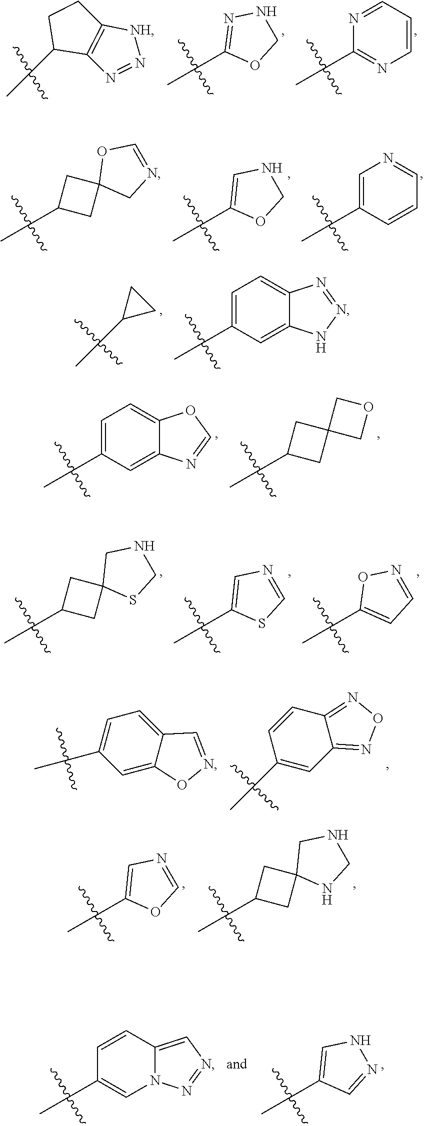

wherein Ring C is substituted with m groups of R^{c}; and all other variables not specifically defined herein are as defined in any one of the suitable preceding embodiments. For example, Ring C is substituted with a group selected from halogen, methyl, trifluoromethyl, OH, NH₂, ═O, ═S, and C(═O)NH₂.

In a 17^{th} embodiment, in a compound, tautomer, a solvate or stereoisomer of the compound or the tautomer, or pharmaceutically acceptable salt of this disclosure, R″ is selected from halogen, CN, C₁ to C₃ alkyl, —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3- to 7-membered carbocyclyl, and 3- to 7-membered heterocyclyl,
wherein:
the C₁ to C₃ alkyl of R^{a} is optionally substituted with 1-2 groups selected from —NR^{p}R^{q}, —C(═O)NR^{p}R^{q}, phenyl, 4- to 7-membered heterocyclyl, halogen, and OR^{s},
the 5- to 6-membered heteroaryl, 3- to 7-membered carbocyclyl, or 3- to 7-membered heterocyclyl of R^{a} is optionally substituted with one group selected from C₁ to C₃ alkyl, —O(C₁ to C₃ alkyl), —N(C₁ to C₃ alkyl)₂, and —NH(C₁ to C₃ alkyl), and
wherein:
   R^{p}, for each occurrence, is independently selected from H and C₁ to C₃ alkyl,
   R^{q}, for each occurrence, is independently selected from H, C₁ to C₃ alkyl, phenyl, 5- to 6-membered heteroaryl, and 4- to 7-membered heterocyclyl, and
   R^{s}, for each occurrence, is independently selected from H, C₁ to C₃ alkyl (which is optionally substituted with 1-2 groups selected from halogen), phenyl, and 4- to 7-membered heterocyclyl,
   wherein:
   the 4- to 7-membered heterocyclyl of R^{q} and R^{s}, in each occurrence, is independently optionally substituted with one group selected from C₁ to C₃ alkyl and —O(C₁ to C₃ alkyl);
      and all other variables not specifically defined herein are as defined in any one of the suitable preceding embodiments.

In a 18^{th} embodiment, in a compound, tautomer, a solvate or stereoisomer of the compound or the tautomer, or pharmaceutically acceptable salt of this disclosure, R^{a} is selected from F, Br, Cl, CN, methyl, ethyl, —C(CH₃)₃, —CH(CH₃)₂, CH₂N(CH₃)₂, CH₂CH₂C(═O)NH₂,

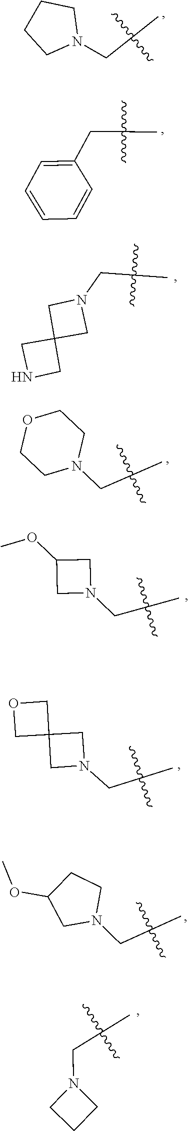

NH₂, —N(CH₃)₂,

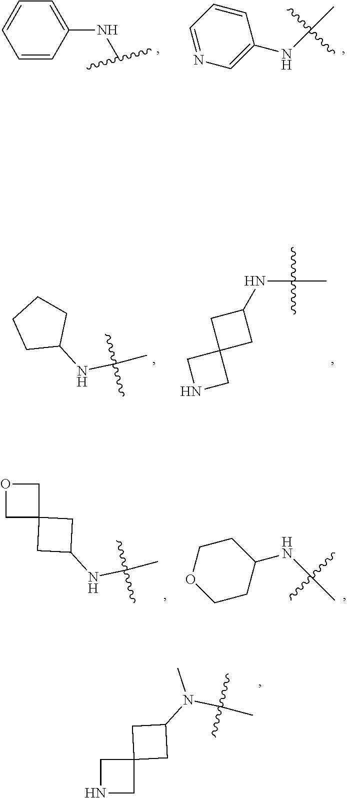

—NHC(═O)CH₃, —NHS(═O)₂CH₃,

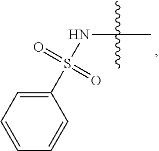 [SMILES: *NS(=O)(=O)c1ccccc1]

### OH, OCH₃,

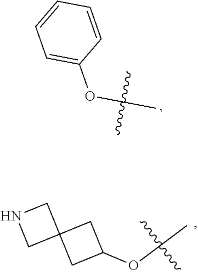 [SMILES: *Oc1ccccc1 ; *OC1CC2(CNC2)C1]

—C(═O)NH₂, —C(═O)NHCH₃,

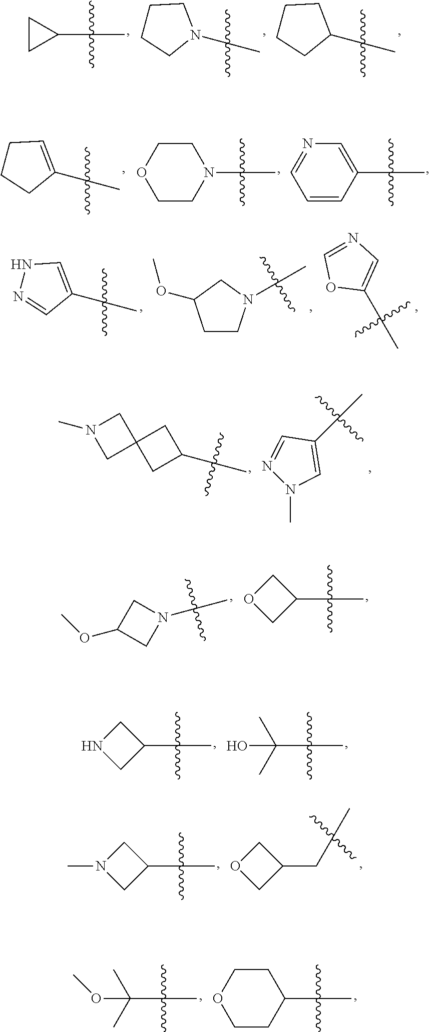

CH₂NHCH₃, CH₂OCH₃, —CHF₂,

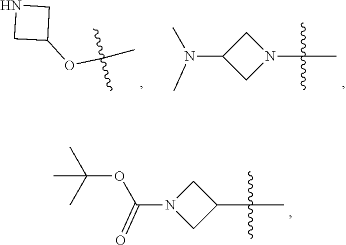

and OCHF₂; and all other variables not specifically defined herein are as defined in any one of the suitable preceding embodiments.

In a 19^{th} embodiment, in a compound, tautomer, a solvate or stereoisomer of the compound or the tautomer, or pharmaceutically acceptable salt of this disclosure, R^{a} is selected from F, Br, Cl, CN, OH, 5- to 6-membered heteroaryl, 3- to 6-membered carbocyclyl, 3- to 6-membered heterocyclyl, and methyl optionally substituted with 3- to 6-membered heterocyclyl; and all other variables not specifically defined herein are as defined in any one of the suitable preceding embodiments.

In a 20^{th} embodiment, in a compound, tautomer, a solvate or stereoisomer of the compound or the tautomer, or pharmaceutically acceptable salt of this disclosure, R^{x} is selected from H, ═O, CN, 3-6 carbocyclyl, C₁ to C₂ alkyl,

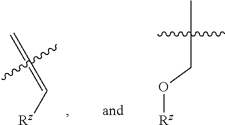

wherein:
the C₁ to C₂ alkyl of R^{x} is optionally substituted with 1-3 groups selected from halogen, deuterium, CN, 5- to 6-membered carbocyclyl, 5- to 9-membered heterocyclyl, 6-membered aryl, 5- to 6-membered heteroaryl, and OH,
   wherein: the 6-membered aryl or the 5- to 6-membered heteroaryl of the C₁ to C₂ alkyl of R^{x} is optionally substituted with 1-3 groups selected from halogen, OR^{s}, CN, CONH₂, NH₂, C₁-C₄ alkyl (which is optionally substituted with 1-3 groups selected from halogen, O—R^{z}, OR^{s}, and 4- to 6-membered heterocyclyl optionally substituted with C₁ to C₂ alkyl), C₁-C₃ alkenyl, C₁-C₃ alkynyl, 3- to 4-membered carbocyclyl, and 4- to 7-membered heterocyclyl (which is optionally substituted with 1-3 groups selected from C₁ to C₂ alkyl, halogen, and OR^{s}) and
   wherein: the 5- to 9-membered heterocyclyl of the C₁ to C₂ alkyl of R^{x} is optionally substituted with 1-2 groups selected from C₁ to C₂ alkyl, ═O, and halogen, wherein:
   R^{s} is selected from H and C₁ to C₂ alkyl optionally substituted with 1-3 groups selected from halogen, O(C₁ to C₂ alkyl), —N(C₁ to C₂ alkyl)(C₁ to C₂ alkyl), and
R^{z} is selected from H, 6-membered aryl, and 5- to 6-membered heteroaryl,
   wherein: the 6-membered aryl or 5- to 6-membered heteroaryl of R^{z} is optionally substituted with 1-2 groups selected from halogen;
      and all other variables not specifically defined herein are as defined in any one of the suitable preceding embodiments.

In a 21^{st} embodiment, in a compound, tautomer, a solvate or stereoisomer of the compound or the tautomer, or pharmaceutically acceptable salt of this disclosure, R^{x} is selected from C₁ to C₂ alkyl, wherein the C₁ to C₂ alkyl of R^{x} is substituted with 6-membered aryl or 5- to 6-membered heteroaryl, wherein: the 6-membered aryl or the 5- to 6-membered heteroaryl of the C₁ to C₂ alkyl of R^{x} is optionally substituted with 1-3 groups selected from halogen, OCH₃, CN, CONH₂, NH₂, C₁-C₂ alkyl (which is optionally substituted with 1-3 groups selected from halogen and O—R^{z}), O(C₁ to C₂ alkyl) (which is optionally substituted with 1-3 groups selected from halogen), 3- to 4-membered carbocyclyl, and 4- to 7-membered heterocyclyl; and all other variables not specifically defined herein are as defined in any one of the suitable preceding embodiments.

In a 22^{nd} embodiment, in a compound, tautomer, a solvate or stereoisomer of the compound or the tautomer, or pharmaceutically acceptable salt of this disclosure, R^{x} is selected from H, ═O, CN, methyl, ethyl, propyl, CH₂CN,

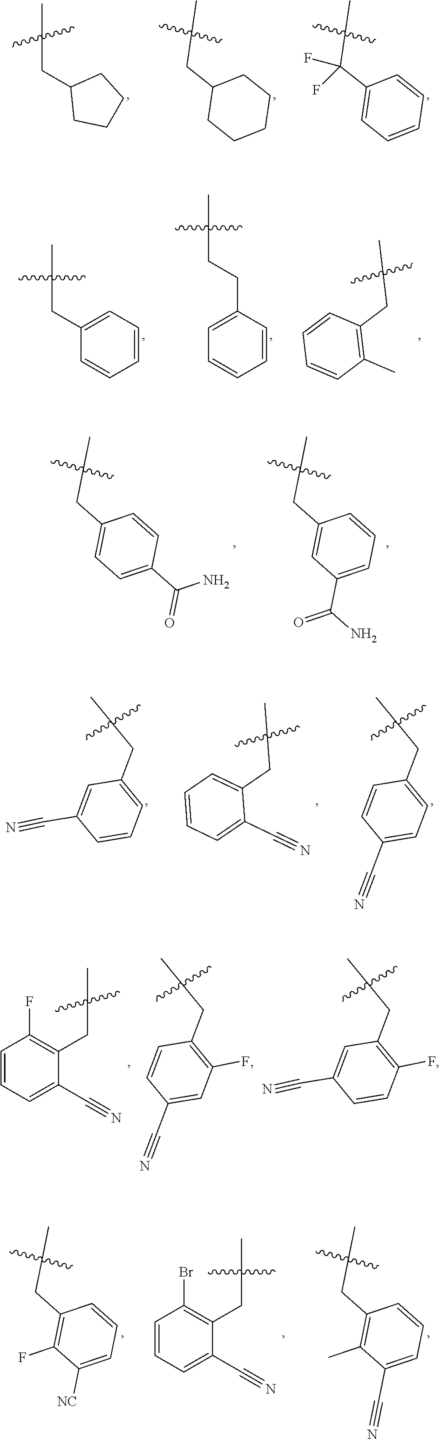

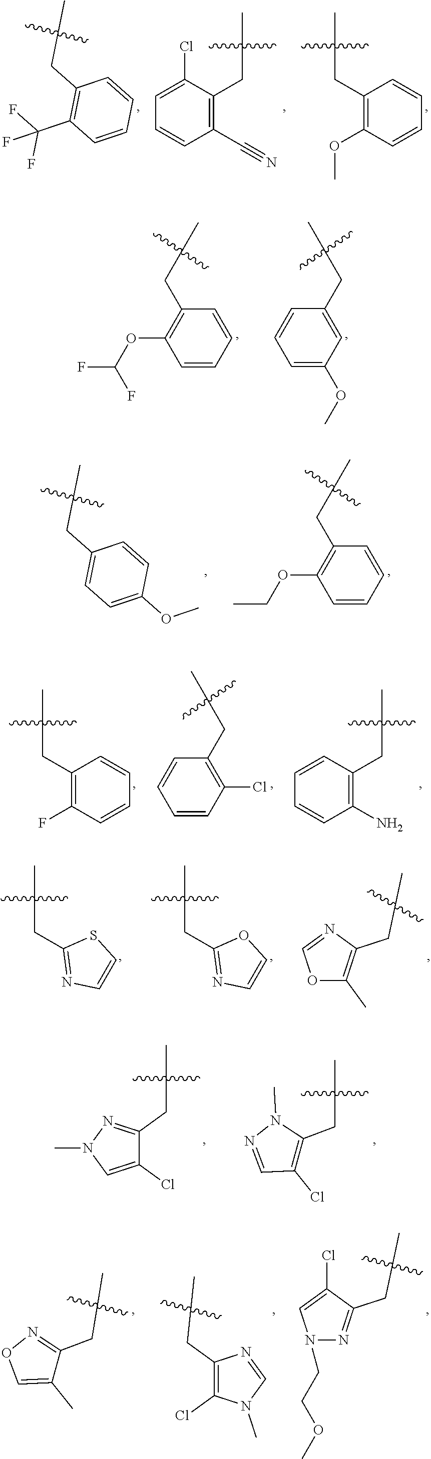

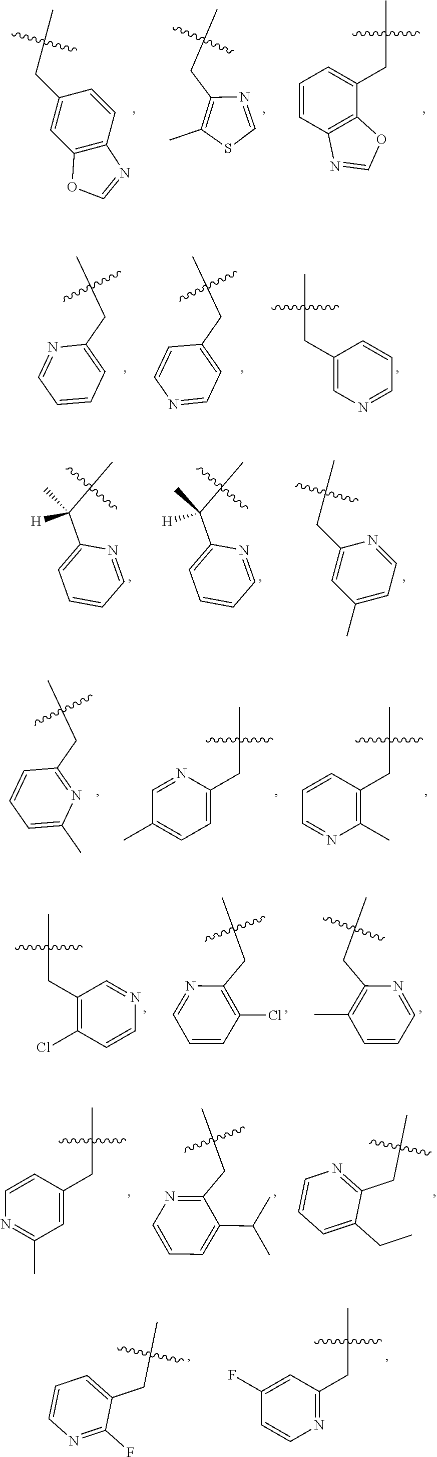

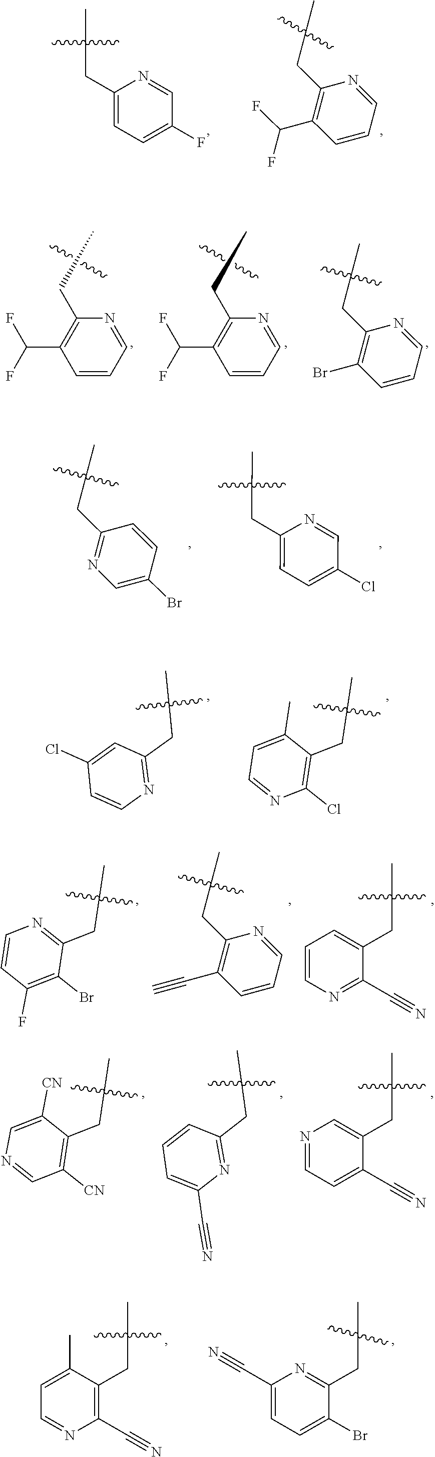

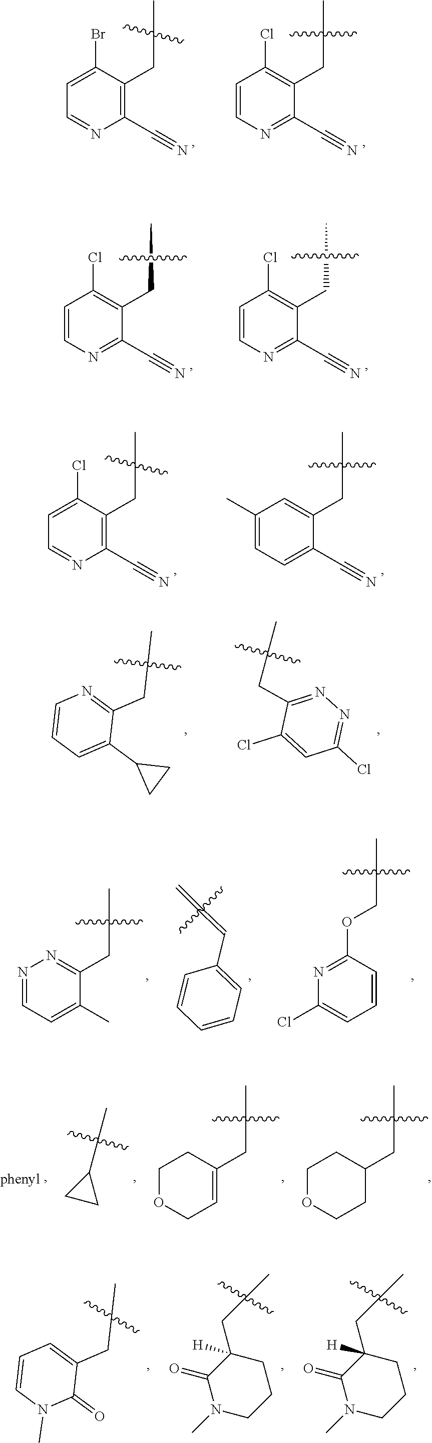

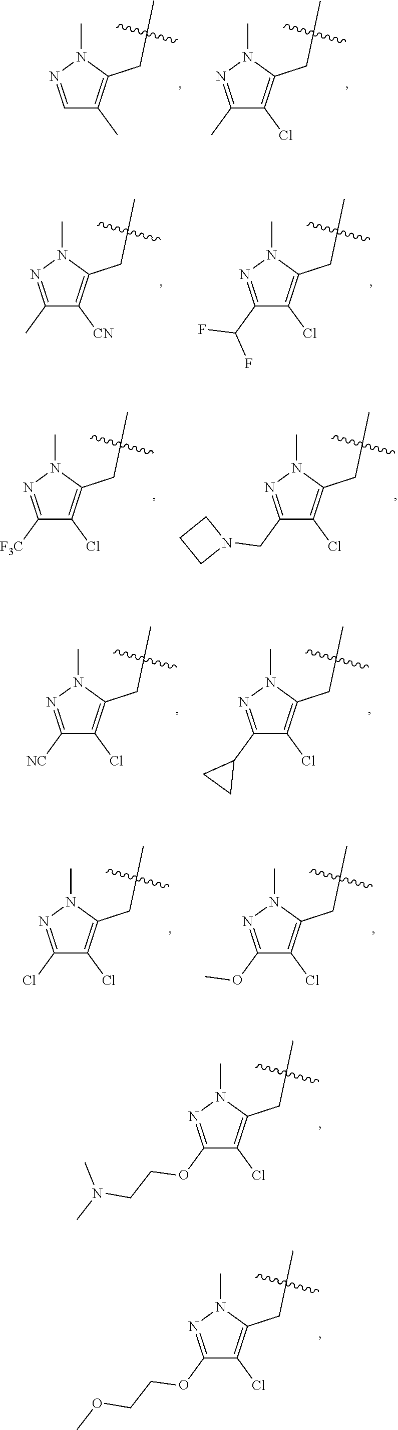

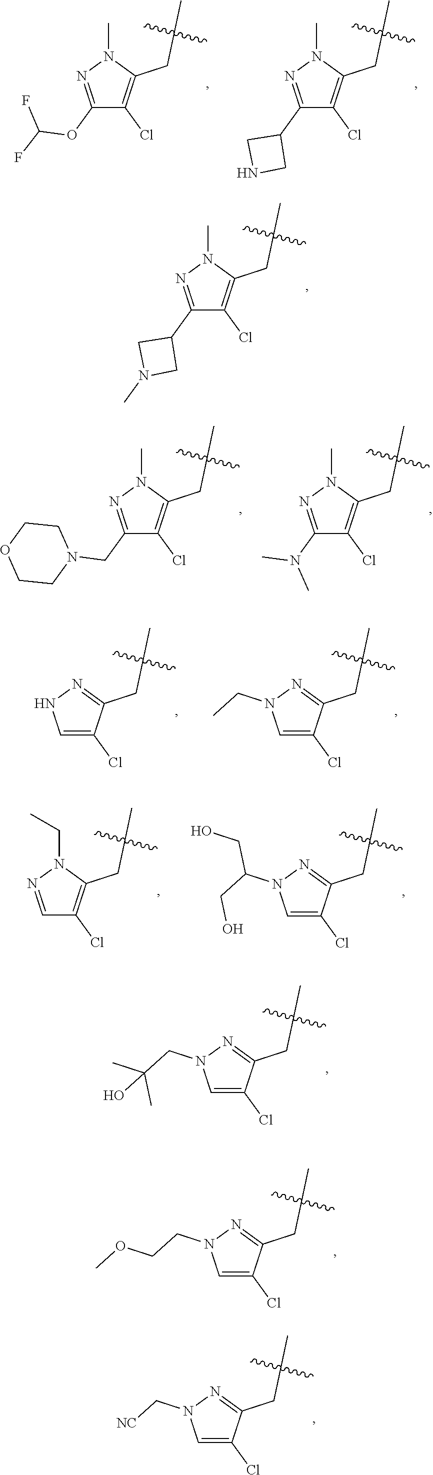

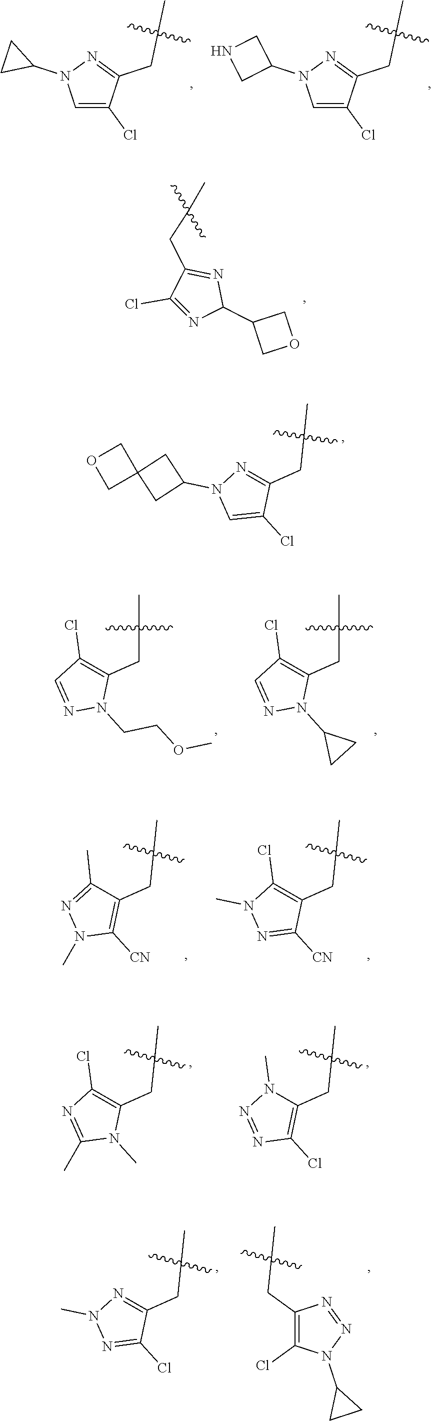

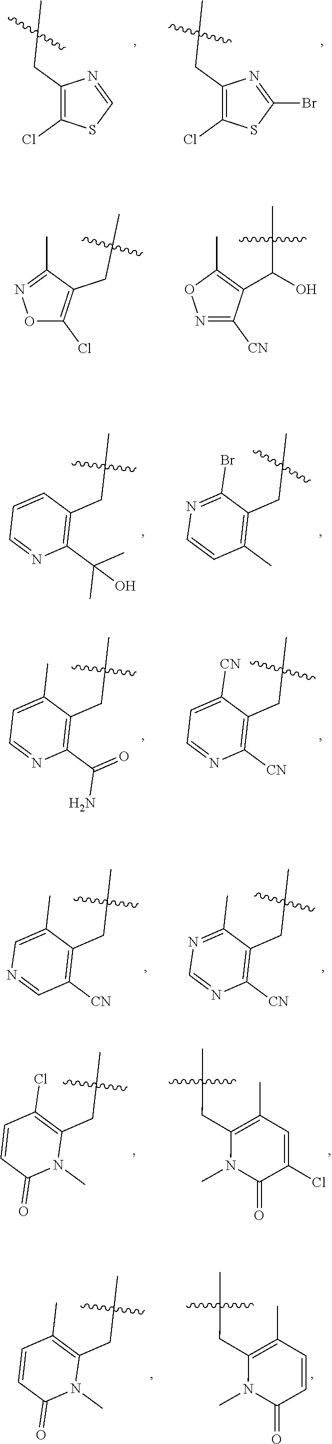

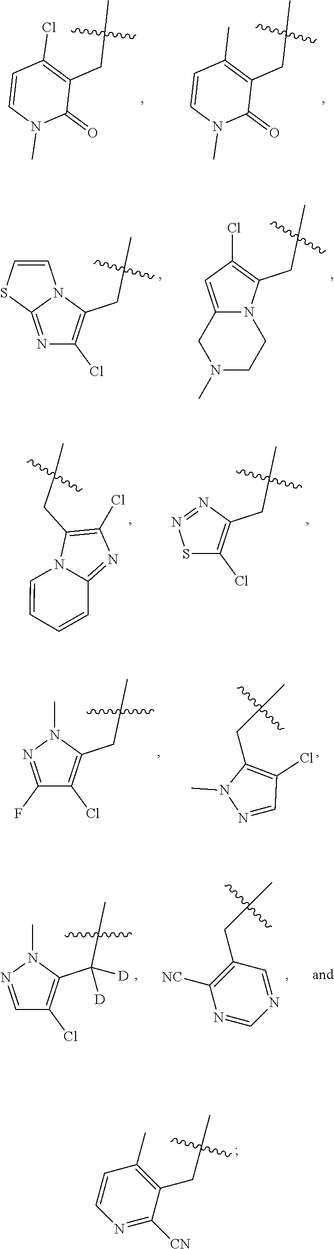

and all other variables not specifically defined herein are as defined in any one of the suitable preceding embodiments.

In a 23^{rd} embodiment, in a compound, tautomer, a solvate or stereoisomer of the compound or the tautomer, or pharmaceutically acceptable salt of this disclosure, R^{s}, for each occurrence, is independently selected from deuterium, halogen, methyl (which is optionally substituted with OR^{s}), trifluoromethyl, ═O, ═S, OR^{s}, —NHC(═O)R^{s}, —NHC(═O)OR^{s}, NR^{p}R^{q}, —NHC(═O)NR^{p}R^{q}, CN, —C(═O)NR^{p}R^{q}, optionally substituted 5- to 6-membered heteroaryl, and optionally substituted 5- to 6-membered heterocyclyl, wherein:
R^{p}, for each occurrence, is independently selected from H and C₁ to C₂ alkyl,
R^{q}, for each occurrence, is independently selected from H, C₁ to C₂ alkyl, phenyl, 5- to 6-membered heteroaryl, 4- to 7-membered heterocyclyl, and CN, and
R^{s}, for each occurrence, is independently selected from H, C₁ to C₂ alkyl (which is optionally substituted with 1-3 groups selected from halogen and deuterium), phenyl, and 4- to 7-membered heterocyclyl,
wherein:
the 4- to 7-membered heterocyclyl of R^{q} and R^{s}, in each occurrence, is optionally substituted with one group selected from C₁ to C₂ alkyl and —O(C₁ to C₂ alkyl), and wherein: m is selected from 0, 1, 2, and 3;
and all other variables not specifically defined herein are as defined in any one of the suitable preceding embodiments.

In a 24^{th} embodiment, in a compound, tautomer, a solvate or stereoisomer of the compound or the tautomer, or pharmaceutically acceptable salt of this disclosure, R^{c}, for each occurrence, is independently selected from deuterium, F, CH₃, CF₃, OH, ═O, ═S, OCH₃, NH₂, —NHC(═O)CH₃, —NHC(═O)NHCH₃, —NHC(═O)NH₂, —NHC(═O)OCH₃,

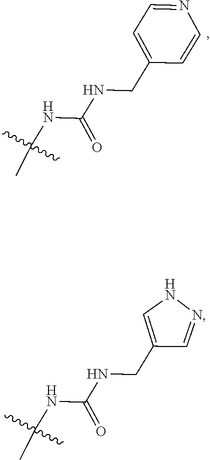 [SMILES: *NC(=O)NCc1ccncc1 ; *NC(=O)NCc1cn[nH]c1]

CN, CH₂OH, —C(═O)NH₂, Cl, —NHCN, OCHF₂,

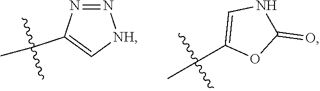 [SMILES: *c1c[nH]nn1 ; *c1c[nH]c(=O)o1]

—SCH₃, OCD₃, and —C(═O)CH₃; and all other variables not specifically defined herein are as defined in any one of the suitable preceding embodiments.

In a 25^{th} embodiment, in a compound, tautomer, a solvate or stereoisomer of the compound or the tautomer, or pharmaceutically acceptable salt of this disclosure, m is zero; and all other variables not specifically defined herein are as defined in any one of the suitable preceding embodiments.

In a 26^{th} embodiment, in a compound, tautomer, a solvate or stereoisomer of the compound or the tautomer, or pharmaceutically acceptable salt of this disclosure, R^{L} is H and R^{y} is H or deuterium; and all other variables not specifically defined herein are as defined in any one of the suitable preceding embodiments.

In a 27^{th} embodiment, in a compound, tautomer, a solvate or stereoisomer of the compound or the tautomer, or pharmaceutically acceptable salt of this disclosure,

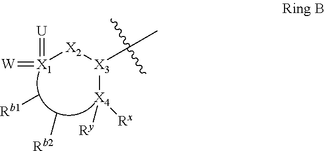

### of Formula 1 is:

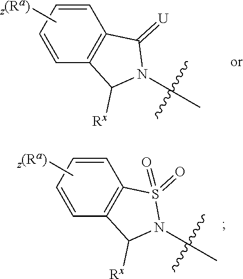

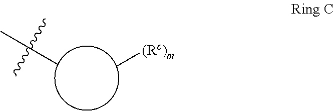

of Formula 1 is selected from:

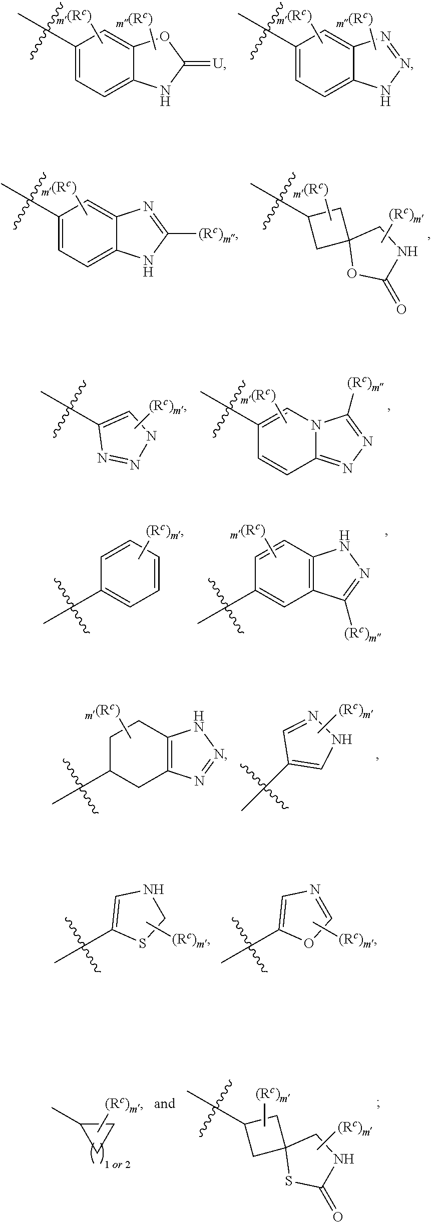

L is selected from —CH₂—, —CD₂-,

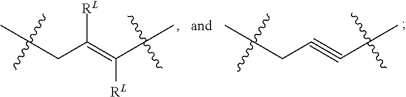

R^{x} is selected from

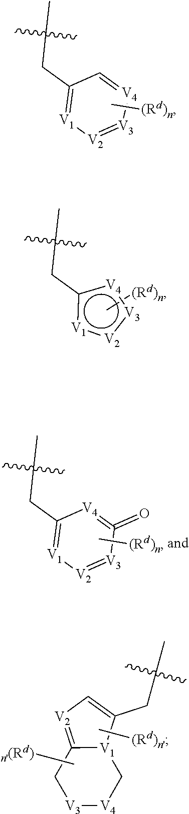

U is O or S; V₁, V₂, V₃, and V₄ are selected from C and N;
R^{L}, for each occurrence, is independently selected from H, deuterium, F, and CH₃; R^{a}, for each occurrence, is selected from CN, halogen, optionally substituted 3- to 4-membered carbocyclyl, optionally substituted 4- to 5-membered heterocyclyl, and optionally substituted O(C₁ to C₃ alkyl);
R^{c}, for each occurrence, is independently selected from deuterium, halogen, OH, C₁ to C₃ alkyl, O(C₁ to C₃ alkyl), and 5- to 6-membered heteroaryl, wherein the O(C₁ to C₃ alkyl) of R^{c} is optionally substituted with 1 to 3 deuteriums;
R^{d}, for each occurrence, is independently selected from halogen, CN, C₁ to C₃ alkyl, and O(C₁ to C₃ alkyl);
z, for each occurrence, is an integer independently selected from 0, 1, 2, and 3;
m′, for each occurrence, is an integer independently selected from 0, 1, and 2;
m″, for each occurrence, is an integer independently selected from 0 and 1;
n, for each occurrence, is an integer independently selected from 0, 1, 2, and 3; and
n′, for each occurrence, is an integer independently selected from 0, 1, and 2; and all other variables not specifically defined herein are as defined in any one of the suitable preceding embodiments.

In a 28^{th} embodiment, in a compound, tautomer, a solvate or stereoisomer of the compound or the tautomer, or pharmaceutically acceptable salt of this disclosure,

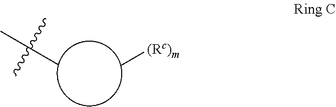

of Formula 1 is selected from:

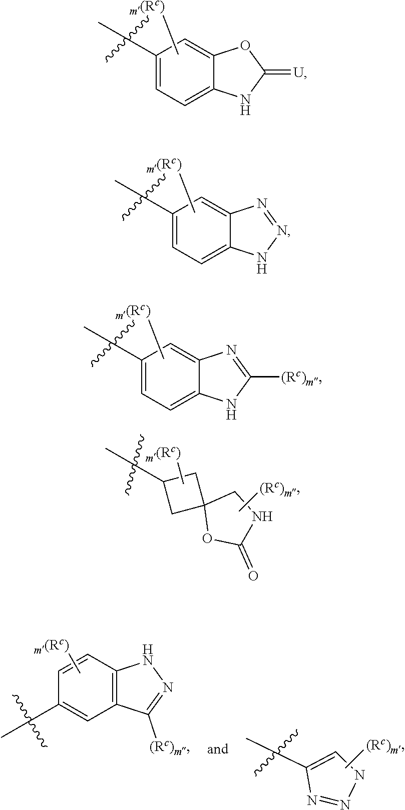

L is selected from —CH₂—, —CD₂—,

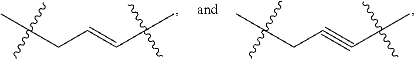 [SMILES: */C=C/C* ; *C#CC*]

R^{x} is selected from

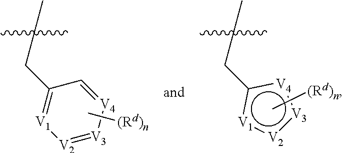

R^{d}, for each occurrence, is independently selected from halogen, CN, C₁ to C₃ alkyl, and O(C₁ to C₃ alkyl);
m′, for each occurrence, is an integer independently selected from 0, 1, and 2;
m″, for each occurrence, is an integer independently selected from 0 and 1; and
n, for each occurrence, is an integer independently selected from 0, 1, 2, and 3; and all other variables not specifically defined herein are as defined in any one of the suitable preceding embodiments.

In a 29^{th} embodiment, in a compound, tautomer, a solvate or stereoisomer of the compound or the tautomer, or pharmaceutically acceptable salt of this disclosure,

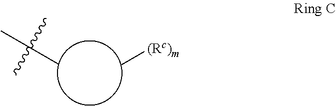

of Formula 1 is selected from:

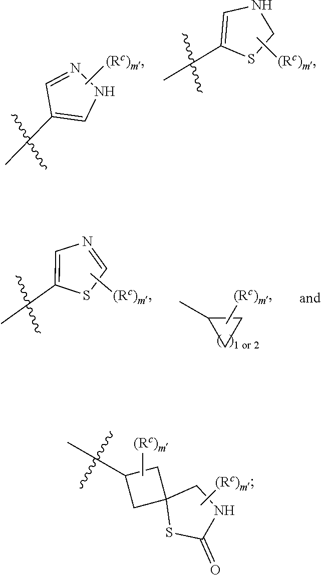

L is selected from —CH₂—, —CD₂—,

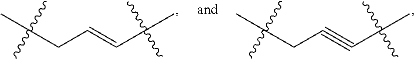 [SMILES: */C=C/C* ; *C#CC*]

R^{x} is selected from

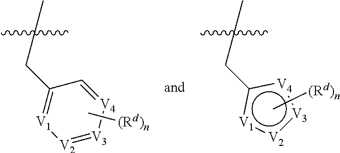

R^{d}, for each occurrence, is independently selected from halogen, CN, C₁ to C₃ alkyl, and O(C₁ to C₃ alkyl);
m′, for each occurrence, is an integer independently selected from 0, 1, and 2; and
n, for each occurrence, is an integer independently selected from 0, 1, 2, and 3; and all other variables not specifically defined herein are as defined in any one of the suitable preceding embodiments.

In a 30^{th} embodiment, in a compound, tautomer, a solvate or stereoisomer of the compound or the tautomer, or pharmaceutically acceptable salt of this disclosure,
U is O;
V₁, V₂, V₃, and V₄ are selected from C and N, wherein no more than two of V₁, V₂, V₃, and V₄ are N;
R^{L} is H or deuterium;
R^{a}, for each occurrence, is independently selected from CN, F, Cl, Br, 3-membered carbocyclyl, 4- to 5-membered heterocyclyl optionally substituted by —N(C₁ to C₂ alkyl)(C₁ to C₂ alkyl), and OCHF₂;
R^{c}, for each occurrence, is independently selected from deuterium, F, OH, CH₃, OCH₃, OCD₃, and

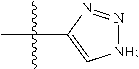 [SMILES: *c1c[nH]nn1]

R^{d}, for each occurrence, is independently selected from F, Cl, Br, CH₃, CH₂CH₃, CN, and OCH₃;
z, for each occurrence, is an integer independently selected from 0, 1, and 2;
m′, for each occurrence, is an integer independently selected from 0, 1, and 2;
n, for each occurrence, is an integer independently selected from 0, 1, 2, and 3; and
n′, for each occurrence, is an integer independently selected from 0, 1, and 2;
   and all other variables not specifically defined herein are as defined in any one of the suitable preceding embodiments.

In a 31^{st} embodiment, a compound of the disclosure is a compound of the following structural Formulae 7a-7e:

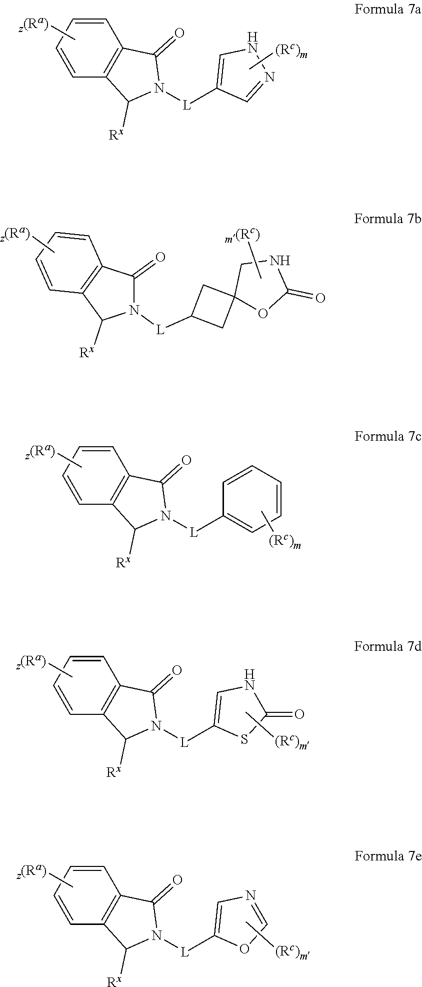

a tautomer thereof, a solvate or stereoisomer of the compound or the tautomer, or a pharmaceutically acceptable salt of the foregoing, wherein L is selected from —CH₂—, —CD₂—,

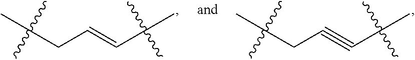 [SMILES: */C=C/C* ; *C#CC*]

m′ is selected from 0, 1, and 2, z is selected from 0, 1, and 2; and all other variables not specifically defined herein are as defined in any one of the suitable preceding embodiments.

In a 32^{nd} embodiment, in a compound, tautomer, a solvate or stereoisomer of the compound or the tautomer, or pharmaceutically acceptable salt of this disclosure,
L is selected from —CH₂— and —CD₂—;
R^{a}, for each occurrence, is independently selected from C₁ to C₃ alkyl, halogen, and O(C₁ to C₃ alkyl) optionally substituted with 1 to 3 groups selected from halogen;
R^{c}, for each occurrence, is independently selected from deuterium, halogen, OH, CH₃, OCH₃, and OCD₃;
R^{d}, for each occurrence, is independently selected from halogen, CN, OCH₃, and C₁ to C₄ alkyl;
R^{x} is selected from

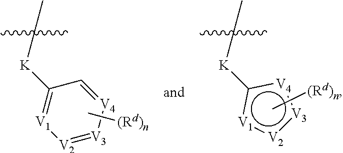

wherein K is selected from —CH₂— and —CD₂—, and
n, for each occurrence, is an integer independently selected from 0, 1, 2 and 3; and all other variables not specifically defined herein are as defined in any one of the suitable preceding embodiments.

In a 33^{rd} embodiment, a compound of the disclosure is a compound of the following structural Formula 8:

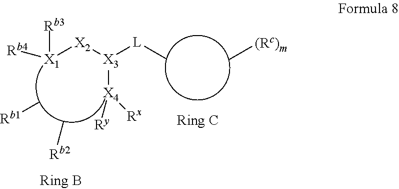

a tautomer thereof, a solvate or stereoisomer of the compound or the tautomer, or a pharmaceutically acceptable salt of the foregoing, wherein:
X₁ is C;
X₂ is C, N, or absent; X₃ is C or N; X₄ is C, N, or absent;
Ring B is a 5- to 6-membered heterocyclic group, wherein Ring B comprises zero or one heteroatom at a position other than the positions of X₁, X₂, X₃, and X₄;
Ring C is phenyl, a 9- to 10-membered aryl, a 5- to 6-membered heteroaryl, a 9- to 10-membered heteroaryl, a 3- to 6-membered carbocyclyl, or a 4- to 12-membered heterocyclic group;
L is selected from

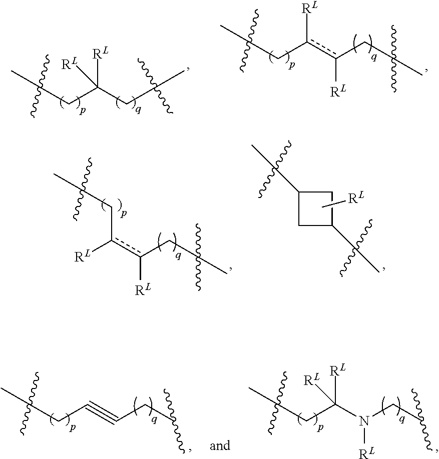

wherein R^{L}, for each occurrence, is independently selected from H, deuterium, halogen, and C₁ to C₃ alkyl optionally substituted with 1-3 groups selected from halogen;
R^{b1} and R^{b2} are attached to two adjacent positions in Ring B and R^{b1} and R^{b2} join to form Ring A, wherein Ring A is a phenyl or 5- to 6-membered heteroaryl group and wherein Ring A is substituted with z groups of R^{a};
R^{b3} and R^{b4} are independently selected from H and C₁ to C₃ alkyl, or R^{b3} and R^{b4} join to form a 3- to 4-membered carbocyclyl,
R^{a}, for each occurrence, is independently selected from halogen, CN, C₁ to C₄ alkyl, —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3- to 7-membered carbocyclyl, and 3- to 7-membered heterocyclyl,
   wherein:
   the C₁ to C₄ alkyl of R^{a} is optionally substituted with 1-3 groups selected from —NR^{p}R^{q}, —C(═O)NR^{p}R^{q}, phenyl, 5- to 6-membered heteroaryl, 3- to 7-membered carbocyclyl, 3- to 7-membered heterocyclyl, halogen, and OR^{s},
   the 5- to 6-membered heteroaryl, the 3- to 7-membered carbocyclyl, or the 3- to 7-membered heterocyclyl of R^{a} is optionally substituted with 1-2 groups selected from C₁ to C₄ alkyl, —O(C₁ to C₄ alkyl), —N(C₁ to C₄ alkyl)₂, and —NH(C₁ to C₄ alkyl);
R^{x} is selected from H, ═O, CN, C₃ to C₆ carbocyclyl, C₁ to C₄ alkyl,

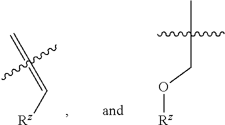

wherein:
   the C₁ to C₄ alkyl of R^{x} is optionally substituted with 1-3 groups selected from halogen, deuterium, CN, 5- to 6-membered carbocyclyl, 5- to 9-membered heterocyclyl, 6-membered aryl, 5- to 6-membered heteroaryl, and OH,
      wherein: the 6-membered aryl or the 5- to 6-membered heteroaryl of the C₁ to C₄ alkyl of R^{x} is optionally substituted with 1-3 groups selected from halogen, OR^{s}, CN, C(═O)NR^{p}R^{q}, NR^{p}R^{q}, C₁-C₆ alkyl (which is optionally substituted with 1-3 groups selected from halogen, O—R^{z}, OR^{s}, and 4- to 6-membered heterocyclyl optionally substituted with C₁ to C₂ alkyl), C₁-C₆ alkenyl, C₁-C₆ alkynyl, 3- to 6-membered carbocyclyl, and 4- to 7-membered heterocyclyl (which is optionally substituted with 1-3 groups selected from optionally substituted C₁ to C₄ alkyl, halogen, and OR^{s}), and
      wherein: the 5- to 9-membered heterocyclyl of the C₁ to C₄ alkyl of R′ is optionally substituted with 1-2 groups selected from C₁ to C₆ alkyl, ═O, and halogen,
   R^{z} is selected from H, 6-membered aryl, and 5- to 6-membered heteroaryl,
      wherein: the 6-membered aryl or 5- to 6-membered heteroaryl of R′ is optionally substituted with 1-2 groups selected from halogen;
R^{y} is selected from H, C₁ to C₂ alkyl, and absent;
R^{c}, for each occurrence, is independently selected from deuterium, halogen, C₁ to C₄ alkyl, ═O, ═S, OR^{s}, —NHC(═O)R^{s}, —NHC(═O)OR^{s}, NR^{p}R^{q}, —NHC(═O)NR^{p}R^{q}, CN, optionally substituted 5- to 6-membered heteroaryl or optionally substituted 5- to 6-membered heterocyclyl, and —C(═O)NR^{p}R^{q},
   wherein:
   the C₁ to C₄ alkyl of R^{c} is optionally substituted with 1-3 groups selected from halogen and OR^{s};
wherein:
   R^{p}, for each occurrence, is independently selected from H and C₁ to C₆ alkyl, R^{q}, for each occurrence, is independently selected from H, C₁ to C₆ alkyl, phenyl, 5- to 6-membered heteroaryl, 4- to 7-membered heterocyclyl, and CN, or
   R^{p} and R^{q} join to form a 3- to 6-membered carbocyclyl;
   R^{s}, for each occurrence, is independently selected from H, C₁ to C, alkyl (which is optionally substituted with 1-3 groups selected from deuterium, halogen, O(C₁ to C₄ alkyl), and NR^{p}R^{q}), phenyl, and 4- to 7-membered heterocyclyl;
   wherein:
   the 4- to 7-membered heterocyclyl of R^{q} and R^{s}, in each occurrence, is optionally substituted with 1-2 groups selected from C₁ to C₄ alkyl and —O(C₁ to C₄ alkyl); and
wherein:
m is an integer selected from 0, 1, 2, and 3;
p is an integer selected from 0, 1, 2, and 3;
q is an integer selected from 0, 1, 2, and 3;
z is an integer selected from 0, 1, 2, 3, and 4;
the sum of p and q is an integer equal to or less than 5; and
all other variables not specifically defined herein are as defined in any one of the suitable preceding embodiments.

In a 34^{th} embodiment, a compound of the disclosure is a compound of the following structural Formulae 9a-9h:

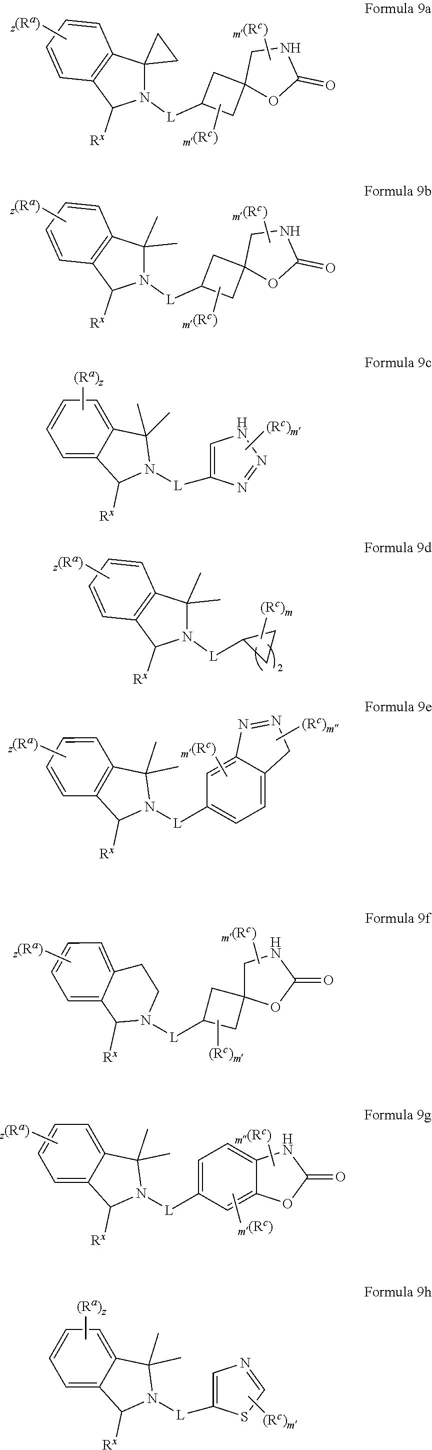

a tautomer thereof, a solvate or stereoisomer of the compound or the tautomer, or a pharmaceutically acceptable salt of the foregoing, wherein:
L is selected from —CH₂—, —CD₂—,

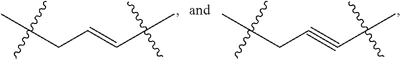 [SMILES: */C=C/C* ; *C#CC*]

m′, for each occurrence, is an integer independently is selected from 0, 1, and 2, m″, for each occurrence, is an integer independently is selected from 0, and 1, z, for each occurrence, is an integer independently is selected from 0, 1, and 2; all other variables not specifically defined herein are as defined in any one of the suitable preceding embodiments.

In a 35^{th} embodiment, in a compound, tautomer, a solvate or stereoisomer of the compound or the tautomer, or pharmaceutically acceptable salt of this disclosure,
L is selected from —CH₂—, —CD₂—, and

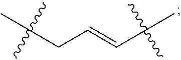 [SMILES: */C=C/C*]

R^{a} is selected from C₁ to C₃ alkyl, CN, and halogen;
R^{x} is selected from

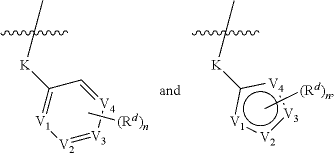

wherein K is selected from —CH₂— and —CD₂—;
R^{c}, for each occurrence, is independently selected from deuterium, C₁ to C₃ alkyl, halogen, 5- to 6-membered heteroaryl, OH, and O(C₁ to C₃ alkyl) optionally substituted with 1 to 3 groups selected from halogen and deuterium,
R^{d}, for each occurrence, is independently selected from halogen, CN, OCH₃, —C(═O)NH₂, and C₁ to C₄ alkyl; and
n, for each occurrence, is an integer independently selected from 0, 1, 2 and 3;
   all other variables not specifically defined herein are as defined in any one of the suitable preceding embodiments.

In a 36^{th} embodiment, in a compound, tautomer, a solvate or stereoisomer of the compound or the tautomer, or pharmaceutically acceptable salt of this disclosure,
R^{a} is selected from F and Cl;
R^{x} is selected from:

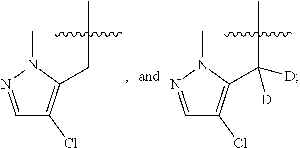

R^{c}, for each occurrence, is independently selected from: deuterium, F, OH, OCH₃, OCD₃, and C(═O)NH₂;
m, for each occurrence, is independently 0, 1, or 2;
m′, for each occurrence, is independently 0, 1 or 2;
z, for each occurrence, is independently 0, 1, or 2;
   all other variables not specifically defined herein are as defined in any one of the suitable preceding embodiments.

In certain embodiments, a compound of the disclosure is selected from Compounds 1 to 645 depicted in Table 1, a tautomer thereof, a solvate or stereoisomer of the compound or the tautomer, or a pharmaceutically acceptable salt of the foregoing.

TABLE 1
Compounds 1 to 645

| | |
|---|---|
| 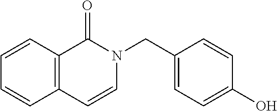 [SMILES: O=c1c2ccccc2ccn1Cc1ccc(O)cc1] | 1 |
| 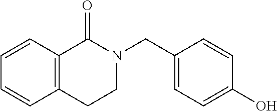 [SMILES: O=C1c2ccccc2CCN1Cc1ccc(O)cc1] | 2 |
| 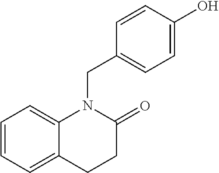 [SMILES: O=C1CCc2ccccc2N1Cc1ccc(O)cc1] | 3 |
| 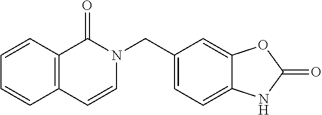 [SMILES: O=c1[nH]c2ccc(Cn3ccc4ccccc4c3=O)cc2o1] | 4 |
| 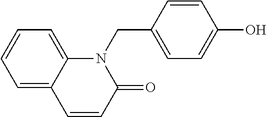 [SMILES: O=c1ccc2ccccc2n1Cc1ccc(O)cc1] | 5 |
| 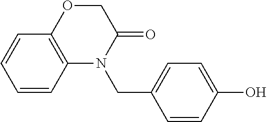 [SMILES: O=C1COc2ccccc2N1Cc1ccc(O)cc1] | 6 |
| 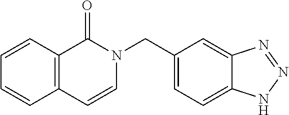 [SMILES: O=c1c2ccccc2ccn1Cc1ccc2[nH]nnc2c1] | 7 |
| 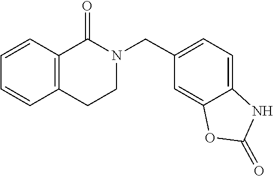 [SMILES: O=C1c2ccccc2CCN1Cc1ccc2[nH]c(=O)oc2c1] | 8 |
| 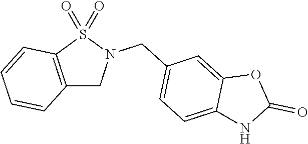 [SMILES: O=c1[nH]c2ccc(CN3Cc4ccccc4S3(=O)=O)cc2o1] | 9 |
| 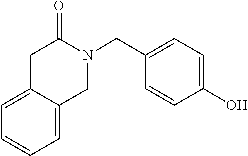 [SMILES: O=C1Cc2ccccc2CN1Cc1ccc(O)cc1] | 10 |
| 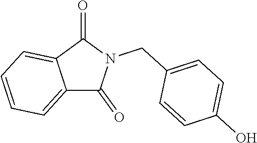 [SMILES: O=C1c2ccccc2C(=O)N1Cc1ccc(O)cc1] | 11 |
| 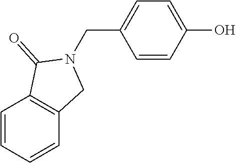 [SMILES: O=C1c2ccccc2CN1Cc1ccc(O)cc1] | 12 |
| 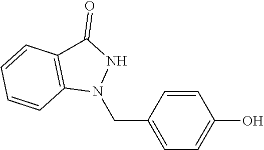 [SMILES: O=c1[nH]n(Cc2ccc(O)cc2)c2ccccc12] | 13 |
| 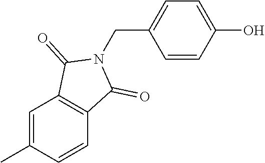 [SMILES: Cc1ccc2c(c1)C(=O)N(Cc1ccc(O)cc1)C2=O] | 14 |
| 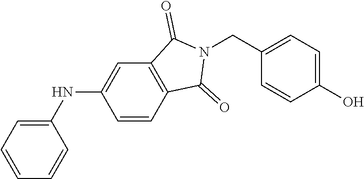 [SMILES: O=C1c2ccc(Nc3ccccc3)cc2C(=O)N1Cc1ccc(O)cc1] | 15 |
| 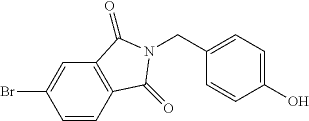 [SMILES: O=C1c2ccc(Br)cc2C(=O)N1Cc1ccc(O)cc1] | 16 |
| 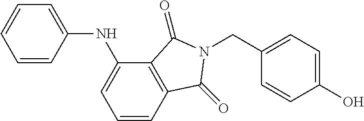 [SMILES: O=C1c2cccc(Nc3ccccc3)c2C(=O)N1Cc1ccc(O)cc1] | 17 |
| 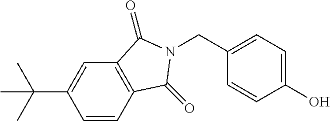 [SMILES: CC(C)(C)c1ccc2c(c1)C(=O)N(Cc1ccc(O)cc1)C2=O] | 18 |
| 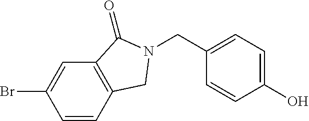 [SMILES: O=C1c2cc(Br)ccc2CN1Cc1ccc(O)cc1] | 19 |
| 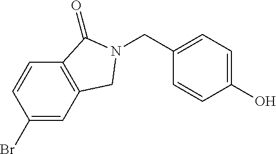 [SMILES: O=C1c2ccc(Br)cc2CN1Cc1ccc(O)cc1] | 20 |
| 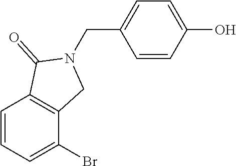 [SMILES: O=C1c2cccc(Br)c2CN1Cc1ccc(O)cc1] | 21 |
| 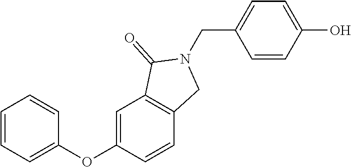 [SMILES: O=C1c2cc(Oc3ccccc3)ccc2CN1Cc1ccc(O)cc1] | 22 |
| 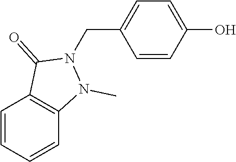 [SMILES: Cn1c2ccccc2c(=O)n1Cc1ccc(O)cc1] | 23 |
| 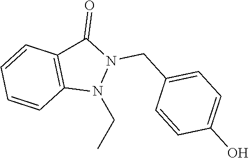 [SMILES: CCn1c2ccccc2c(=O)n1Cc1ccc(O)cc1] | 24 |
| 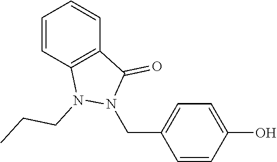 [SMILES: CCCn1c2ccccc2c(=O)n1Cc1ccc(O)cc1] | 25 |
| 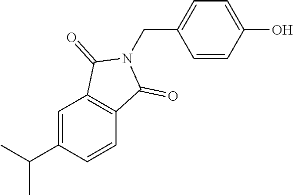 [SMILES: CC(C)c1ccc2c(c1)C(=O)N(Cc1ccc(O)cc1)C2=O] | 26 |
| 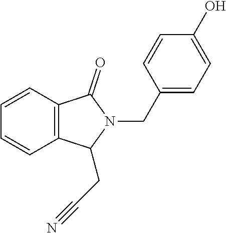 [SMILES: N#CCC1c2ccccc2C(=O)N1Cc1ccc(O)cc1] | 27 |
| 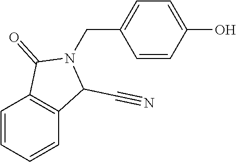 [SMILES: N#CC1c2ccccc2C(=O)N1Cc1ccc(O)cc1] | 28 |
| 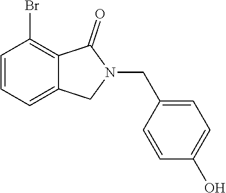 [SMILES: O=C1c2c(Br)cccc2CN1Cc1ccc(O)cc1] | 29 |
| 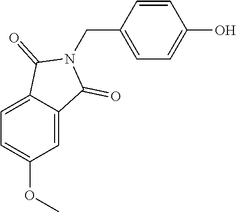 [SMILES: COc1ccc2c(c1)C(=O)N(Cc1ccc(O)cc1)C2=O] | 30 |
| 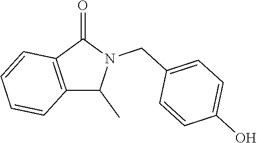 [SMILES: CC1c2ccccc2C(=O)N1Cc1ccc(O)cc1] | 31 |
| 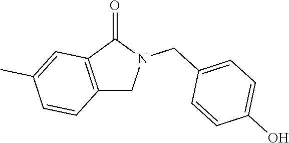 [SMILES: Cc1ccc2c(c1)C(=O)N(Cc1ccc(O)cc1)C2] | 32 |
| 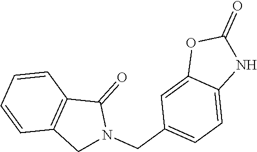 [SMILES: O=C1c2ccccc2CN1Cc1ccc2[nH]c(=O)oc2c1] | 33 |
| 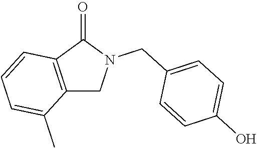 [SMILES: Cc1cccc2c1CN(Cc1ccc(O)cc1)C2=O] | 34 |
| 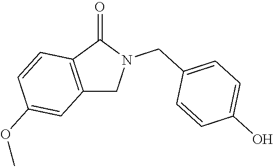 [SMILES: COc1ccc2c(c1)CN(Cc1ccc(O)cc1)C2=O] | 35 |
| 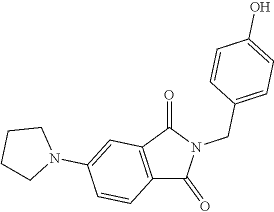 [SMILES: O=C1c2ccc(N3CCCC3)cc2C(=O)N1Cc1ccc(O)cc1] | 36 |
| 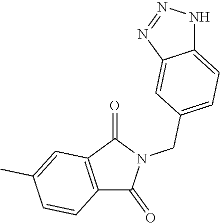 [SMILES: Cc1ccc2c(c1)C(=O)N(Cc1ccc3[nH]nnc3c1)C2=O] | 37 |
| 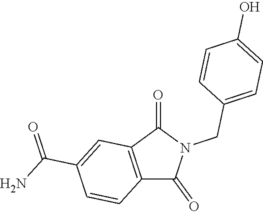 [SMILES: NC(=O)c1ccc2c(c1)C(=O)N(Cc1ccc(O)cc1)C2=O] | 38 |
| 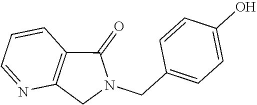 [SMILES: O=C1c2cccnc2CN1Cc1ccc(O)cc1] | 39 |
| 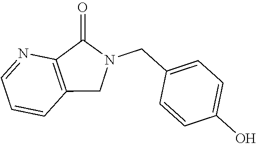 [SMILES: O=C1c2ncccc2CN1Cc1ccc(O)cc1] | 40 |
| 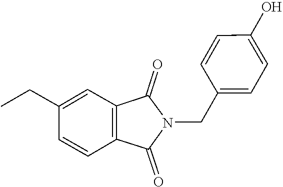 [SMILES: CCc1ccc2c(c1)C(=O)N(Cc1ccc(O)cc1)C2=O] | 41 |
| 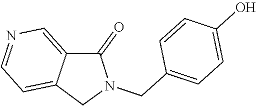 [SMILES: O=C1c2cnccc2CN1Cc1ccc(O)cc1] | 42 |
| 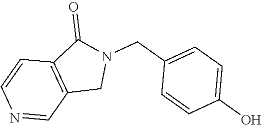 [SMILES: O=C1c2ccncc2CN1Cc1ccc(O)cc1] | 43 |
| 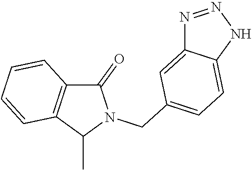 [SMILES: CC1c2ccccc2C(=O)N1Cc1ccc2[nH]nnc2c1] | 44 |
| 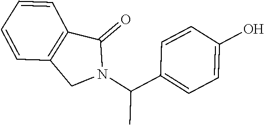 [SMILES: CC(c1ccc(O)cc1)N1Cc2ccccc2C1=O] | 45 |
| 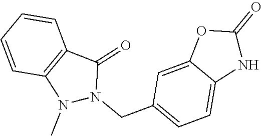 [SMILES: Cn1c2ccccc2c(=O)n1Cc1ccc2[nH]c(=O)oc2c1] | 46 |
| 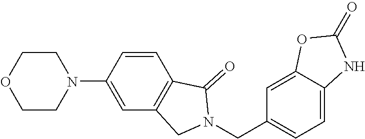 [SMILES: O=C1c2ccc(N3CCOCC3)cc2CN1Cc1ccc2[nH]c(=O)oc2c1] | 47 |
| 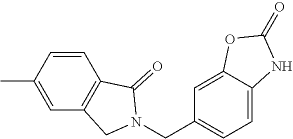 [SMILES: Cc1ccc2c(c1)CN(Cc1ccc3[nH]c(=O)oc3c1)C2=O] | 48 |
| 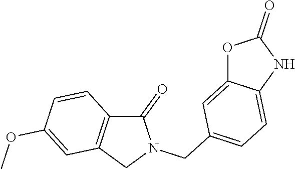 [SMILES: COc1ccc2c(c1)CN(Cc1ccc3[nH]c(=O)oc3c1)C2=O] | 49 |
| 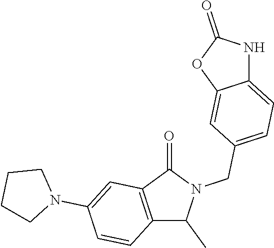 [SMILES: CC1c2ccc(N3CCCC3)cc2C(=O)N1Cc1ccc2[nH]c(=O)oc2c1] | 50 |
| 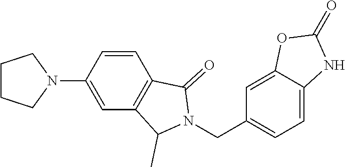 [SMILES: CC1c2cc(N3CCCC3)ccc2C(=O)N1Cc1ccc2[nH]c(=O)oc2c1] | 51 |
| 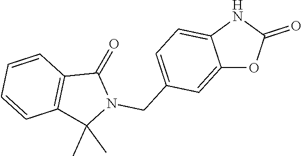 [SMILES: CC1(C)c2ccccc2C(=O)N1Cc1ccc2[nH]c(=O)oc2c1] | 52 |
| 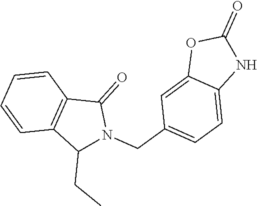 [SMILES: CCC1c2ccccc2C(=O)N1Cc1ccc2[nH]c(=O)oc2c1] | 53 |
| 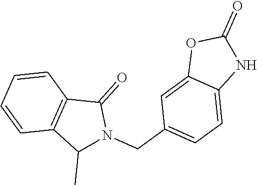 [SMILES: CC1c2ccccc2C(=O)N1Cc1ccc2[nH]c(=O)oc2c1] | 54 |
| 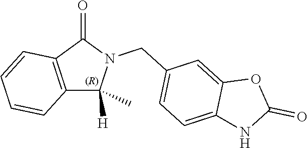 | 55 |
| 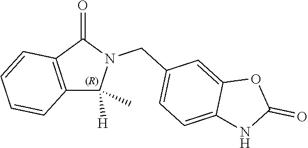 | 56 |
| 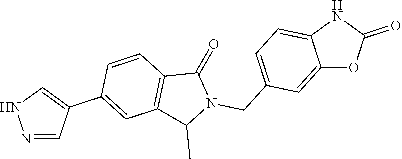 [SMILES: CC1c2cc(-c3cn[nH]c3)ccc2C(=O)N1Cc1ccc2[nH]c(=O)oc2c1] | 57 |
| 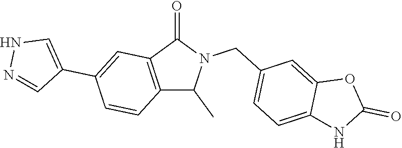 [SMILES: CC1c2ccc(-c3cn[nH]c3)cc2C(=O)N1Cc1ccc2[nH]c(=O)oc2c1] | 58 |
| 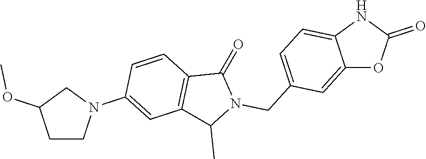 [SMILES: COC1CCN(c2ccc3c(c2)C(C)N(Cc2ccc4[nH]c(=O)oc4c2)C3=O)C1] | 59 |
| 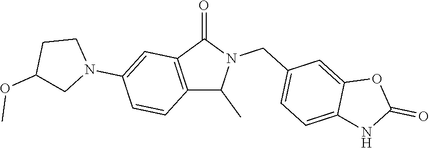 [SMILES: COC1CCN(c2ccc3c(c2)C(=O)N(Cc2ccc4[nH]c(=O)oc4c2)C3C)C1] | 60 |
| 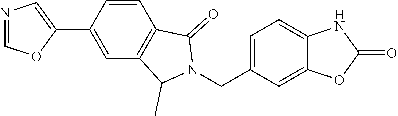 [SMILES: CC1c2cc(-c3cnco3)ccc2C(=O)N1Cc1ccc2[nH]c(=O)oc2c1] | 61 |
| 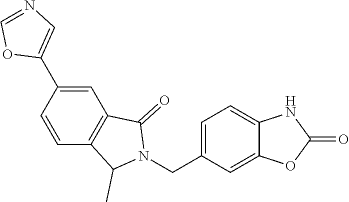 [SMILES: CC1c2ccc(-c3cnco3)cc2C(=O)N1Cc1ccc2[nH]c(=O)oc2c1] | 62 |
| 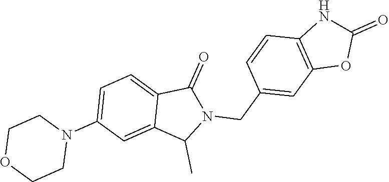 [SMILES: CC1c2cc(N3CCOCC3)ccc2C(=O)N1Cc1ccc2[nH]c(=O)oc2c1] | 63 |
| 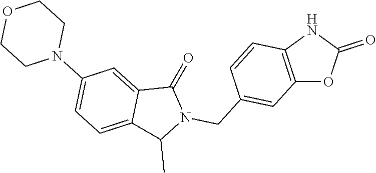 [SMILES: CC1c2ccc(N3CCOCC3)cc2C(=O)N1Cc1ccc2[nH]c(=O)oc2c1] | 64 |
| 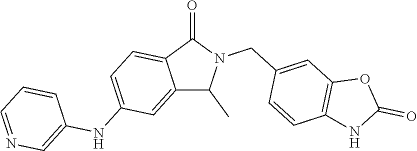 [SMILES: CC1c2cc(Nc3cccnc3)ccc2C(=O)N1Cc1ccc2[nH]c(=O)oc2c1] | 65 |
| 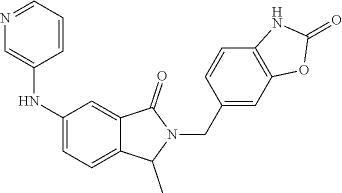 [SMILES: CC1c2ccc(Nc3cccnc3)cc2C(=O)N1Cc1ccc2[nH]c(=O)oc2c1] | 66 |
| 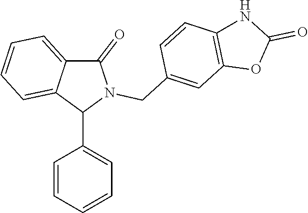 [SMILES: O=C1c2ccccc2C(c2ccccc2)N1Cc1ccc2[nH]c(=O)oc2c1] | 67 |
| 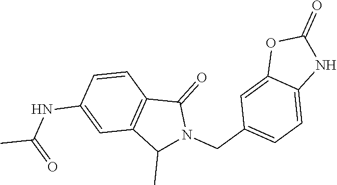 [SMILES: CC(=O)Nc1ccc2c(c1)C(C)N(Cc1ccc3[nH]c(=O)oc3c1)C2=O] | 68 |
| 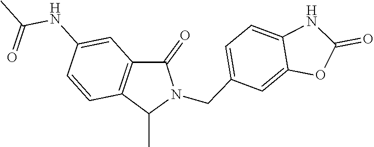 [SMILES: CC(=O)Nc1ccc2c(c1)C(=O)N(Cc1ccc3[nH]c(=O)oc3c1)C2C] | 69 |
| 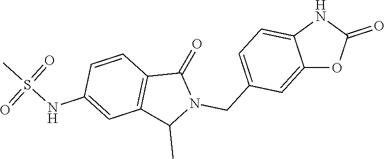 [SMILES: CC1c2cc(NS(C)(=O)=O)ccc2C(=O)N1Cc1ccc2[nH]c(=O)oc2c1] | 70 |
| 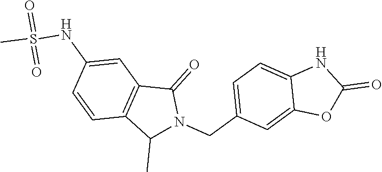 [SMILES: CC1c2ccc(NS(C)(=O)=O)cc2C(=O)N1Cc1ccc2[nH]c(=O)oc2c1] | 71 |
| 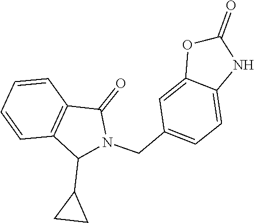 [SMILES: O=C1c2ccccc2C(C2CC2)N1Cc1ccc2[nH]c(=O)oc2c1] | 72 |
| 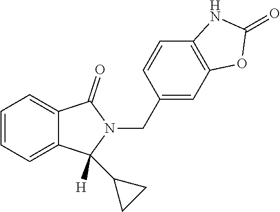 [SMILES: O=C1c2ccccc2[C@@H](C2CC2)N1Cc1ccc2[nH]c(=O)oc2c1] | 73 |
| 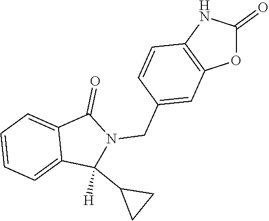 [SMILES: O=C1c2ccccc2[C@H](C2CC2)N1Cc1ccc2[nH]c(=O)oc2c1] | 74 |
| 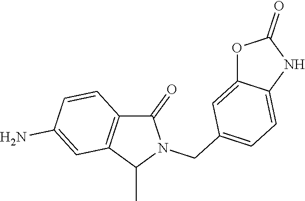 [SMILES: CC1c2cc(N)ccc2C(=O)N1Cc1ccc2[nH]c(=O)oc2c1] | 75 |
| 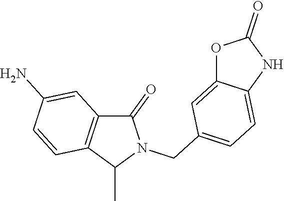 [SMILES: CC1c2ccc(N)cc2C(=O)N1Cc1ccc2[nH]c(=O)oc2c1] | 76 |
| 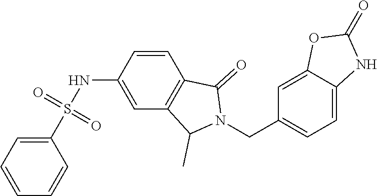 [SMILES: CC1c2cc(NS(=O)(=O)c3ccccc3)ccc2C(=O)N1Cc1ccc2[nH]c(=O)oc2c1] | 77 |
| 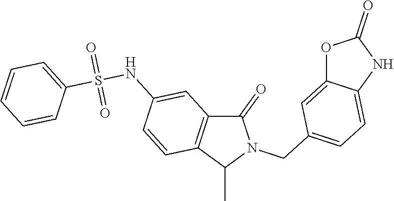 [SMILES: CC1c2ccc(NS(=O)(=O)c3ccccc3)cc2C(=O)N1Cc1ccc2[nH]c(=O)oc2c1] | 78 |
| 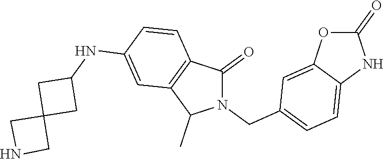 [SMILES: CC1c2cc(NC3CC4(CNC4)C3)ccc2C(=O)N1Cc1ccc2[nH]c(=O)oc2c1] | 79 |
| 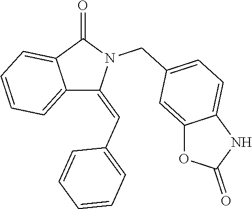 [SMILES: O=C1c2ccccc2/C(=C\c2ccccc2)N1Cc1ccc2[nH]c(=O)oc2c1] | 80-1 |
| 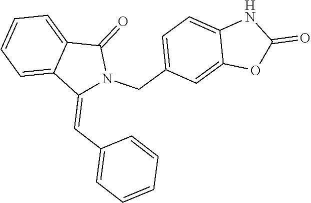 [SMILES: O=C1c2ccccc2/C(=C/c2ccccc2)N1Cc1ccc2[nH]c(=O)oc2c1] | 80-2 |
| 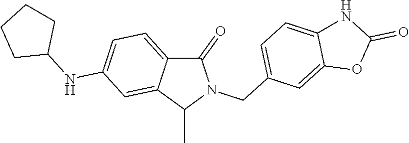 [SMILES: CC1c2cc(NC3CCCC3)ccc2C(=O)N1Cc1ccc2[nH]c(=O)oc2c1] | 81 |
| 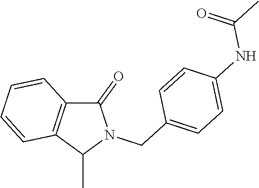 [SMILES: CC(=O)Nc1ccc(CN2C(=O)c3ccccc3C2C)cc1] | 82 |
| 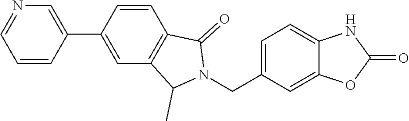 [SMILES: CC1c2cc(-c3cccnc3)ccc2C(=O)N1Cc1ccc2[nH]c(=O)oc2c1] | 83 |
| 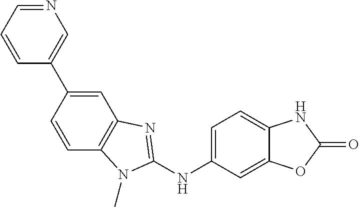 [SMILES: Cn1c(Nc2ccc3[nH]c(=O)oc3c2)nc2cc(-c3cccnc3)ccc21] | 84 |
| 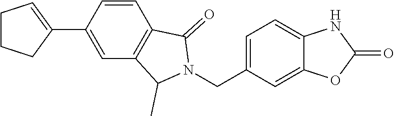 [SMILES: CC1c2cc(C3=CCCC3)ccc2C(=O)N1Cc1ccc2[nH]c(=O)oc2c1] | 85 |
| 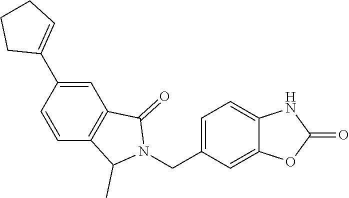 [SMILES: CC1c2ccc(C3=CCCC3)cc2C(=O)N1Cc1ccc2[nH]c(=O)oc2c1] | 86 |
| 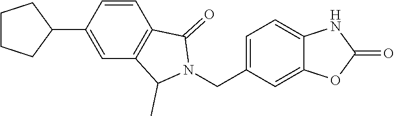 [SMILES: CC1c2cc(C3CCCC3)ccc2C(=O)N1Cc1ccc2[nH]c(=O)oc2c1] | 87 |
| 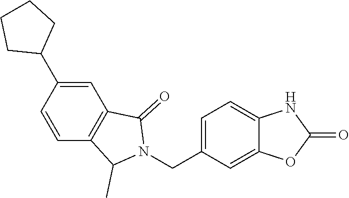 [SMILES: CC1c2ccc(C3CCCC3)cc2C(=O)N1Cc1ccc2[nH]c(=O)oc2c1] | 88 |
| 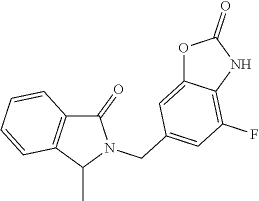 [SMILES: CC1c2ccccc2C(=O)N1Cc1cc(F)c2[nH]c(=O)oc2c1] | 89 |
| 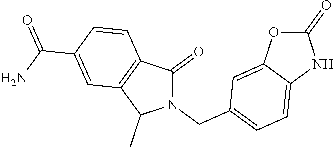 [SMILES: CC1c2cc(C(N)=O)ccc2C(=O)N1Cc1ccc2[nH]c(=O)oc2c1] | 90 |
| 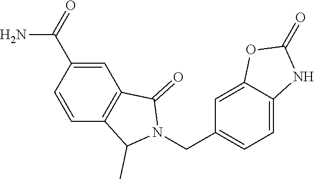 [SMILES: CC1c2ccc(C(N)=O)cc2C(=O)N1Cc1ccc2[nH]c(=O)oc2c1] | 91 |
| 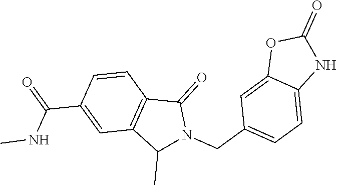 [SMILES: CNC(=O)c1ccc2c(c1)C(C)N(Cc1ccc3[nH]c(=O)oc3c1)C2=O] | 92 |
| 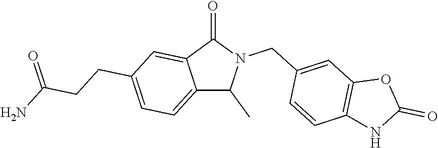 [SMILES: CC1c2ccc(CCC(N)=O)cc2C(=O)N1Cc1ccc2[nH]c(=O)oc2c1] | 93 |
| 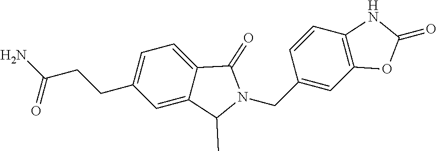 [SMILES: CC1c2cc(CCC(N)=O)ccc2C(=O)N1Cc1ccc2[nH]c(=O)oc2c1] | 94 |
| 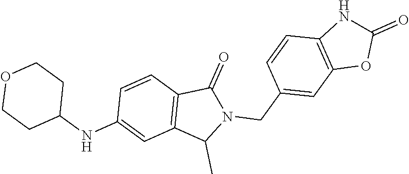 [SMILES: CC1c2cc(NC3CCOCC3)ccc2C(=O)N1Cc1ccc2[nH]c(=O)oc2c1] | 95 |
| 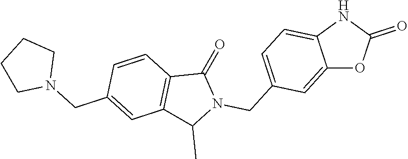 [SMILES: CC1c2cc(CN3CCCC3)ccc2C(=O)N1Cc1ccc2[nH]c(=O)oc2c1] | 96 |
| 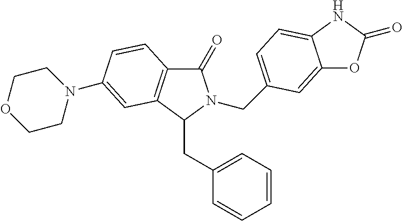 [SMILES: O=C1c2ccc(N3CCOCC3)cc2C(Cc2ccccc2)N1Cc1ccc2[nH]c(=O)oc2c1] | 97 |
| 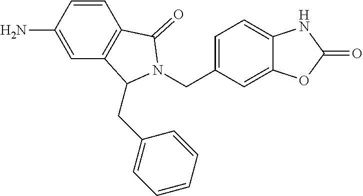 [SMILES: Nc1ccc2c(c1)C(Cc1ccccc1)N(Cc1ccc3[nH]c(=O)oc3c1)C2=O] | 98 |
| 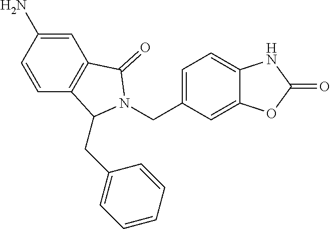 [SMILES: Nc1ccc2c(c1)C(=O)N(Cc1ccc3[nH]c(=O)oc3c1)C2Cc1ccccc1] | 99 |
| 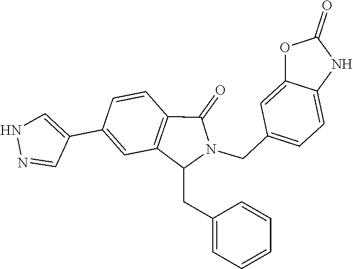 [SMILES: O=C1c2ccc(-c3cn[nH]c3)cc2C(Cc2ccccc2)N1Cc1ccc2[nH]c(=O)oc2c1] | 100 |
| 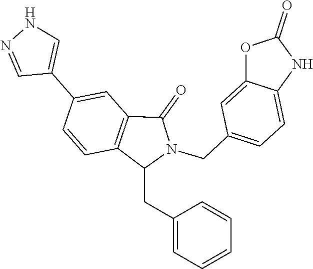 [SMILES: O=C1c2cc(-c3cn[nH]c3)ccc2C(Cc2ccccc2)N1Cc1ccc2[nH]c(=O)oc2c1] | 101 |
| 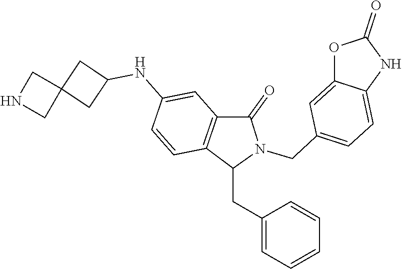 [SMILES: O=C1c2cc(NC3CC4(CNC4)C3)ccc2C(Cc2ccccc2)N1Cc1ccc2[nH]c(=O)oc2c1] | 102 |
| 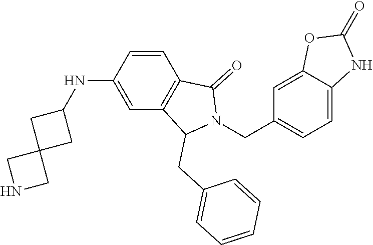 [SMILES: O=C1c2ccc(NC3CC4(CNC4)C3)cc2C(Cc2ccccc2)N1Cc1ccc2[nH]c(=O)oc2c1] | 103 |
| 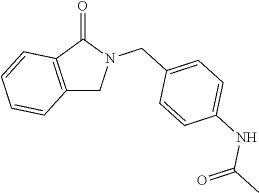 [SMILES: CC(=O)Nc1ccc(CN2Cc3ccccc3C2=O)cc1] | 104 |
| 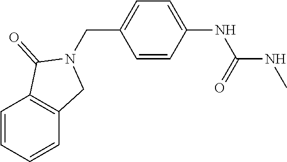 [SMILES: CNC(=O)Nc1ccc(CN2Cc3ccccc3C2=O)cc1] | 105 |
| 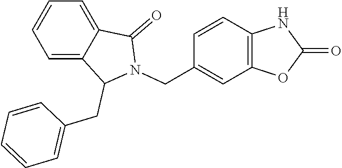 [SMILES: O=C1c2ccccc2C(Cc2ccccc2)N1Cc1ccc2[nH]c(=O)oc2c1] | 106 |
| 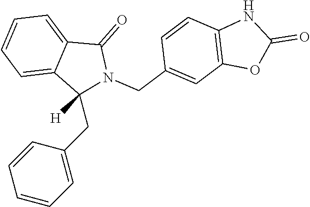 [SMILES: O=C1c2ccccc2[C@@H](Cc2ccccc2)N1Cc1ccc2[nH]c(=O)oc2c1] | 107 |
| 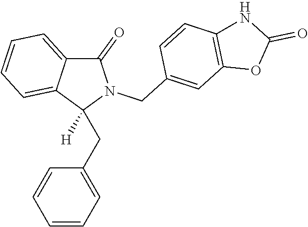 | 108 |
| 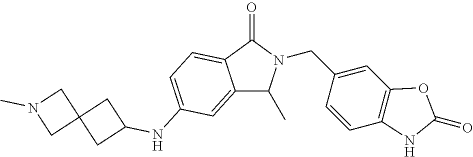 | 109 |
| 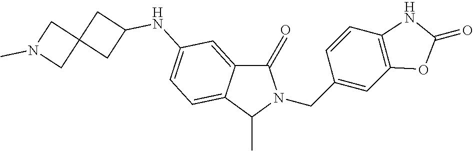 | 110 |
| 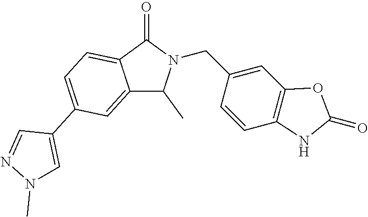 | 111 |
| 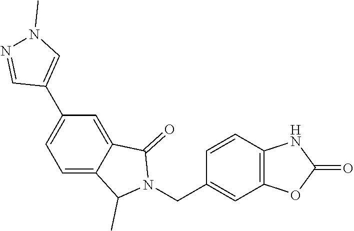 [SMILES: CC1c2ccc(-c3cnn(C)c3)cc2C(=O)N1Cc1ccc2[nH]c(=O)oc2c1] | 112 |
| 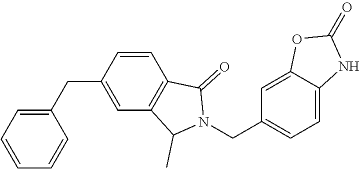 [SMILES: CC1c2cc(Cc3ccccc3)ccc2C(=O)N1Cc1ccc2[nH]c(=O)oc2c1] | 113 |
| 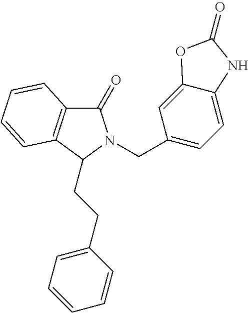 [SMILES: O=C1c2ccccc2C(CCc2ccccc2)N1Cc1ccc2[nH]c(=O)oc2c1] | 114 |
| 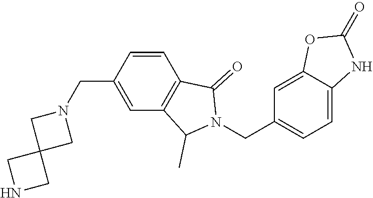 [SMILES: CC1c2cc(CN3CC4(CNC4)C3)ccc2C(=O)N1Cc1ccc2[nH]c(=O)oc2c1] | 115 |
| 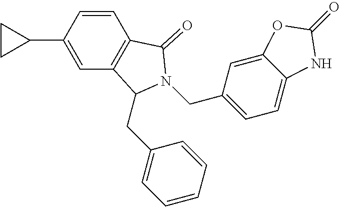 [SMILES: O=C1c2ccc(C3CC3)cc2C(Cc2ccccc2)N1Cc1ccc2[nH]c(=O)oc2c1] | 116 |
| 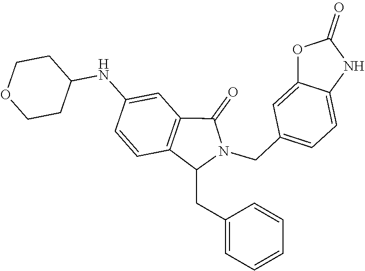 | 117 |
| 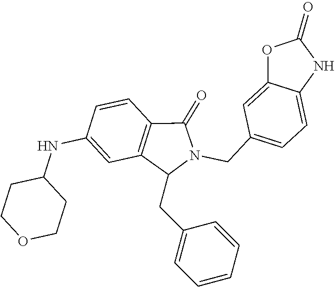 [SMILES: O=C1c2ccc(NC3CCOCC3)cc2C(Cc2ccccc2)N1Cc1ccc2[nH]c(=O)oc2c1] | 118 |
| 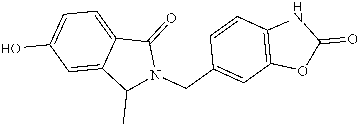 | 119 |
| 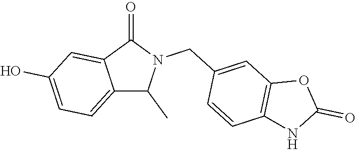 [SMILES: CC1c2ccc(O)cc2C(=O)N1Cc1ccc2[nH]c(=O)oc2c1] | 120 |
| 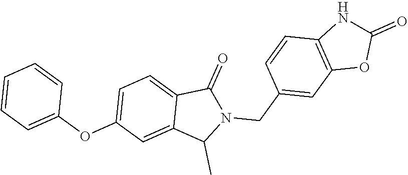 [SMILES: CC1c2cc(Oc3ccccc3)ccc2C(=O)N1Cc1ccc2[nH]c(=O)oc2c1] | 121 |
| 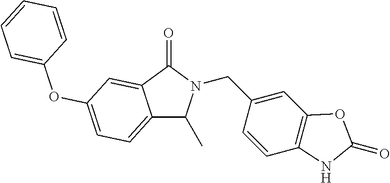 | 122 |
| 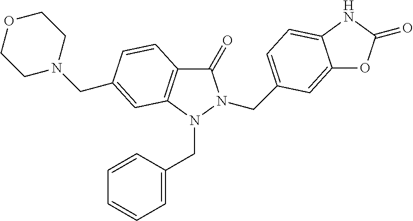 [SMILES: O=c1[nH]c2ccc(Cn3c(=O)c4ccc(CN5CCOCC5)cc4n3Cc3ccccc3)cc2o1] | 123 |
| 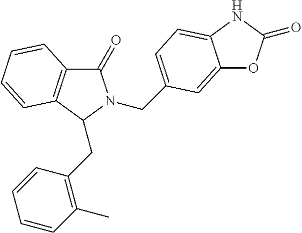 [SMILES: Cc1ccccc1CC1c2ccccc2C(=O)N1Cc1ccc2[nH]c(=O)oc2c1] | 124 |
| 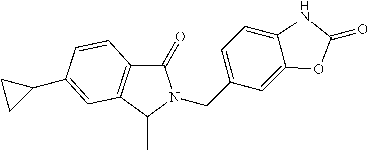 [SMILES: CC1c2cc(C3CC3)ccc2C(=O)N1Cc1ccc2[nH]c(=O)oc2c1] | 125 |
| 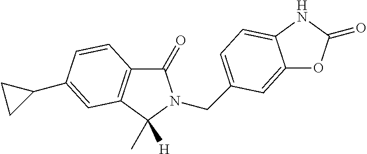 [SMILES: C[C@@H]1c2cc(C3CC3)ccc2C(=O)N1Cc1ccc2[nH]c(=O)oc2c1] | 126 |
| 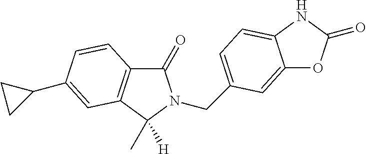 [SMILES: C[C@H]1c2cc(C3CC3)ccc2C(=O)N1Cc1ccc2[nH]c(=O)oc2c1] | 127 |
| 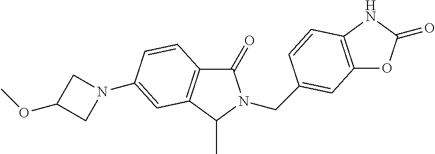 [SMILES: COC1CN(c2ccc3c(c2)C(C)N(Cc2ccc4[nH]c(=O)oc4c2)C3=O)C1] | 128 |
| 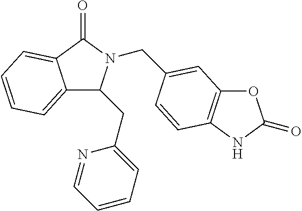 [SMILES: O=C1c2ccccc2C(Cc2ccccn2)N1Cc1ccc2[nH]c(=O)oc2c1] | 129 |
| 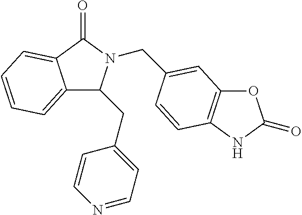 [SMILES: O=C1c2ccccc2C(Cc2ccncc2)N1Cc1ccc2[nH]c(=O)oc2c1] | 130 |
| 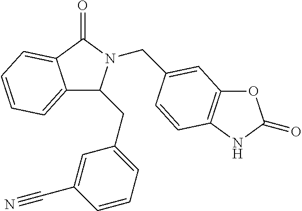 [SMILES: N#Cc1cccc(CC2c3ccccc3C(=O)N2Cc2ccc3[nH]c(=O)oc3c2)c1] | 131 |
| 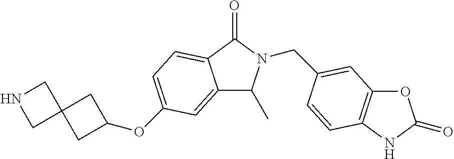 [SMILES: CC1c2cc(OC3CC4(CNC4)C3)ccc2C(=O)N1Cc1ccc2[nH]c(=O)oc2c1] | 132 |
| 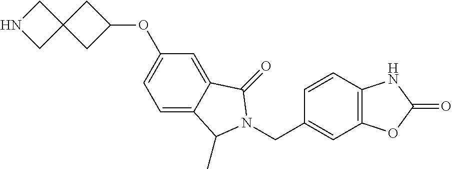 [SMILES: CC1c2ccc(OC3CC4(CNC4)C3)cc2C(=O)N1Cc1ccc2[nH]c(=O)oc2c1] | 133 |
| 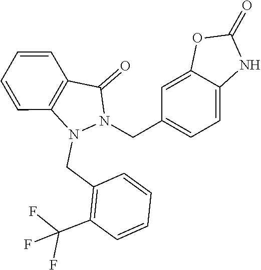 [SMILES: O=c1[nH]c2ccc(Cn3c(=O)c4ccccc4n3Cc3ccccc3C(F)(F)F)cc2o1] | 134 |
| 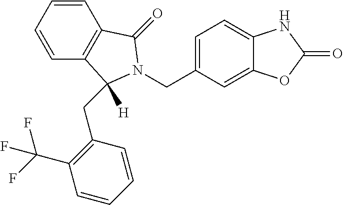 [SMILES: O=C1c2ccccc2[C@@H](Cc2ccccc2C(F)(F)F)N1Cc1ccc2[nH]c(=O)oc2c1] | 135 |
| 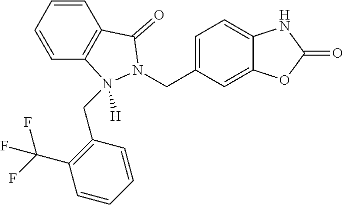 | 136 |
| 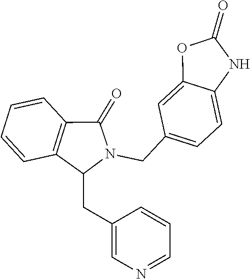 [SMILES: O=C1c2ccccc2C(Cc2cccnc2)N1Cc1ccc2[nH]c(=O)oc2c1] | 137 |
| 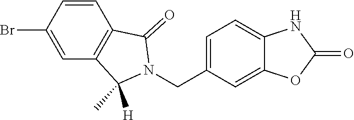 [SMILES: C[C@@H]1c2cc(Br)ccc2C(=O)N1Cc1ccc2[nH]c(=O)oc2c1] | 138 |
| 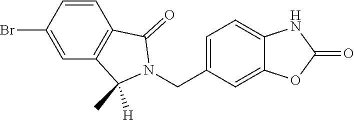 [SMILES: C[C@H]1c2cc(Br)ccc2C(=O)N1Cc1ccc2[nH]c(=O)oc2c1] | 139 |
| 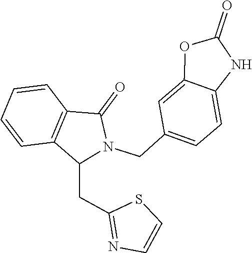 [SMILES: O=C1c2ccccc2C(Cc2nccs2)N1Cc1ccc2[nH]c(=O)oc2c1] | 140 |
| 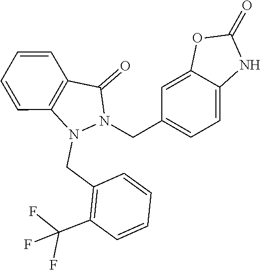 [SMILES: O=c1[nH]c2ccc(Cn3c(=O)c4ccccc4n3Cc3ccccc3C(F)(F)F)cc2o1] | 141 |
| 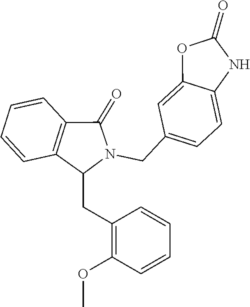 [SMILES: COc1ccccc1CC1c2ccccc2C(=O)N1Cc1ccc2[nH]c(=O)oc2c1] | 142 |
| 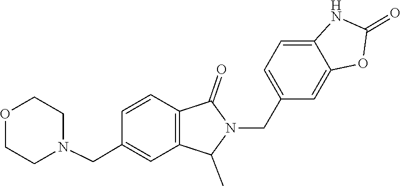 [SMILES: CC1c2cc(CN3CCOCC3)ccc2C(=O)N1Cc1ccc2[nH]c(=O)oc2c1] | 143 |
| 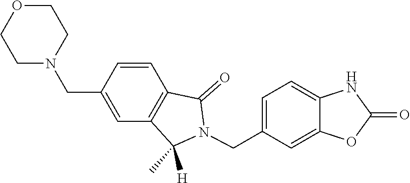 [SMILES: C[C@@H]1c2cc(CN3CCOCC3)ccc2C(=O)N1Cc1ccc2[nH]c(=O)oc2c1] | 144 |
| 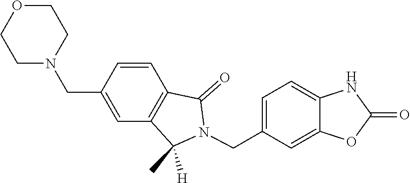 [SMILES: C[C@H]1c2cc(CN3CCOCC3)ccc2C(=O)N1Cc1ccc2[nH]c(=O)oc2c1] | 145 |
| 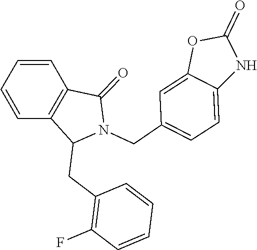 [SMILES: O=C1c2ccccc2C(Cc2ccccc2F)N1Cc1ccc2[nH]c(=O)oc2c1] | 146 |
| 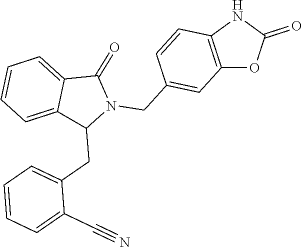 [SMILES: N#Cc1ccccc1CC1c2ccccc2C(=O)N1Cc1ccc2[nH]c(=O)oc2c1] | 147 |
| 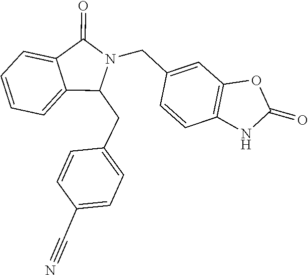 [SMILES: N#Cc1ccc(CC2c3ccccc3C(=O)N2Cc2ccc3[nH]c(=O)oc3c2)cc1] | 148 |
| 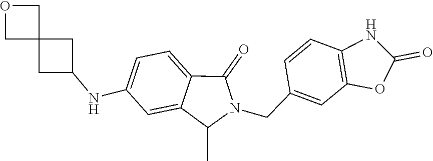 [SMILES: CC1c2cc(NC3CC4(COC4)C3)ccc2C(=O)N1Cc1ccc2[nH]c(=O)oc2c1] | 149 |
| 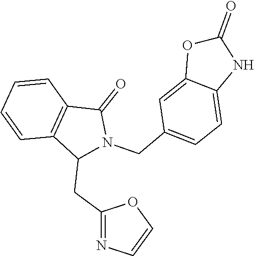 [SMILES: O=C1c2ccccc2C(Cc2ncco2)N1Cc1ccc2[nH]c(=O)oc2c1] | 150 |
| 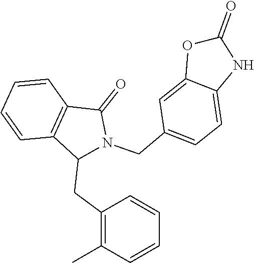 [SMILES: Cc1ccccc1CC1c2ccccc2C(=O)N1Cc1ccc2[nH]c(=O)oc2c1] | 151 |
| 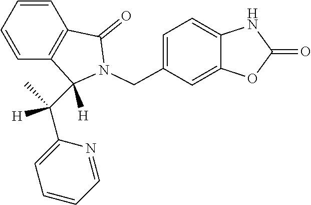 [SMILES: C[C@@H](c1ccccn1)[C@@H]1c2ccccc2C(=O)N1Cc1ccc2[nH]c(=O)oc2c1] | 152 |
| 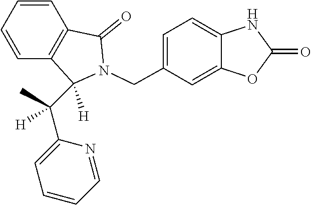 [SMILES: C[C@H](c1ccccn1)[C@H]1c2ccccc2C(=O)N1Cc1ccc2[nH]c(=O)oc2c1] | 153 |
| 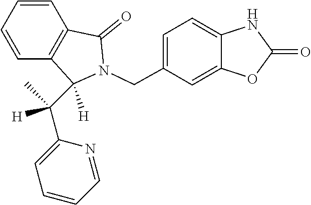 [SMILES: C[C@@H](c1ccccn1)[C@H]1c2ccccc2C(=O)N1Cc1ccc2[nH]c(=O)oc2c1] | 154 |
| 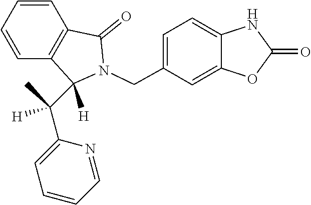 [SMILES: C[C@H](c1ccccn1)[C@@H]1c2ccccc2C(=O)N1Cc1ccc2[nH]c(=O)oc2c1] | 155 |
| 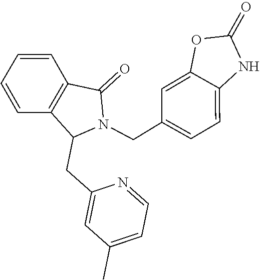 [SMILES: Cc1ccnc(CC2c3ccccc3C(=O)N2Cc2ccc3[nH]c(=O)oc3c2)c1] | 156 |
| 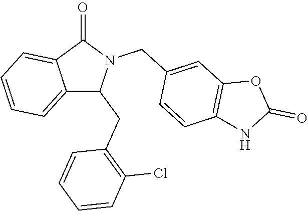 | 157 |
| 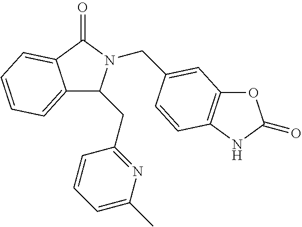 | 158 |
| 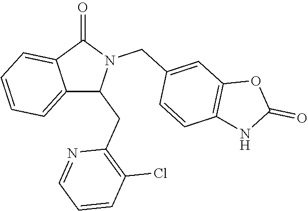 | 159 |
| 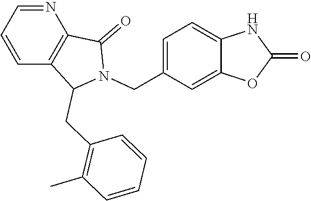 | 160 |
| 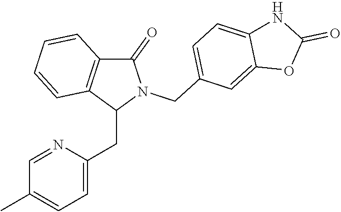 | 161 |
| 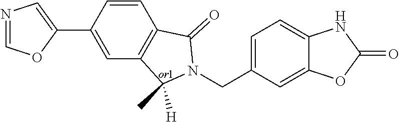 | 162 |
| 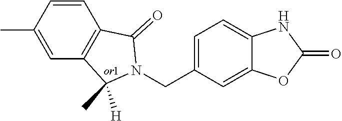 | 163 |
| 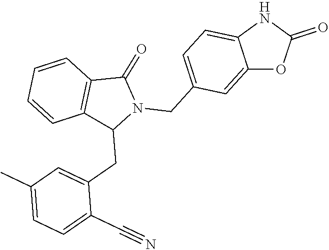 | 164 |
| 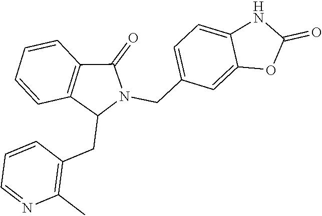 | 165 |
| 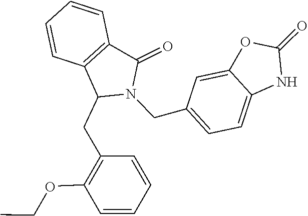 [SMILES: CCOc1ccccc1CC1c2ccccc2C(=O)N1Cc1ccc2[nH]c(=O)oc2c1] | 166 |
| 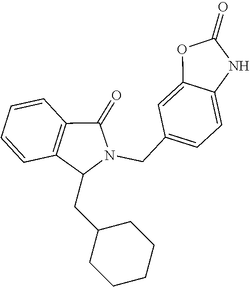 [SMILES: O=C1c2ccccc2C(CC2CCCCC2)N1Cc1ccc2[nH]c(=O)oc2c1] | 167 |
| 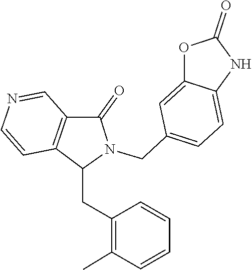 [SMILES: Cc1ccccc1CC1c2ccncc2C(=O)N1Cc1ccc2[nH]c(=O)oc2c1] | 168 |
| 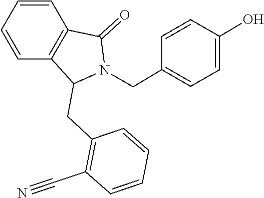 [SMILES: N#Cc1ccccc1CC1c2ccccc2C(=O)N1Cc1ccc(O)cc1] | 169 |
| 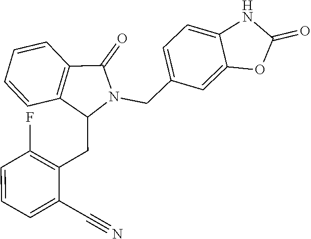 [SMILES: N#Cc1cccc(F)c1CC1c2ccccc2C(=O)N1Cc1ccc2[nH]c(=O)oc2c1] | 170 |
| 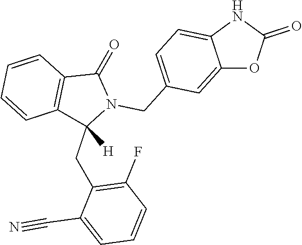 [SMILES: N#Cc1cccc(F)c1C[C@@H]1c2ccccc2C(=O)N1Cc1ccc2[nH]c(=O)oc2c1] | 171 |
| 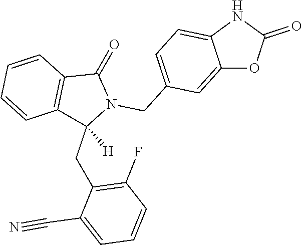 [SMILES: N#Cc1cccc(F)c1C[C@H]1c2ccccc2C(=O)N1Cc1ccc2[nH]c(=O)oc2c1] | 172 |
| 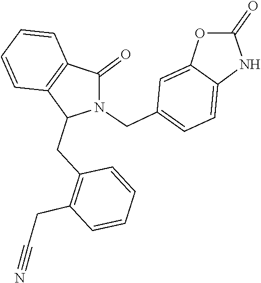 [SMILES: N#CCc1ccccc1CC1c2ccccc2C(=O)N1Cc1ccc2[nH]c(=O)oc2c1] | 173 |
| 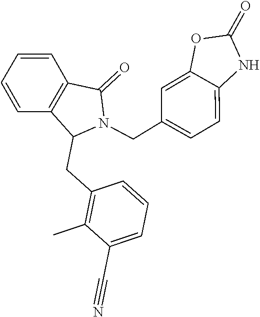 [SMILES: Cc1c(C#N)cccc1CC1c2ccccc2C(=O)N1Cc1ccc2[nH]c(=O)oc2c1] | 174 |
| 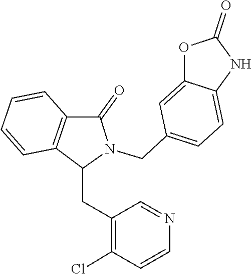 [SMILES: O=C1c2ccccc2C(Cc2cnccc2Cl)N1Cc1ccc2[nH]c(=O)oc2c1] | 175 |
| 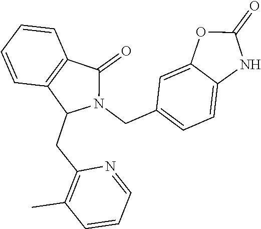 [SMILES: Cc1cccnc1CC1c2ccccc2C(=O)N1Cc1ccc2[nH]c(=O)oc2c1] | 176 |
| 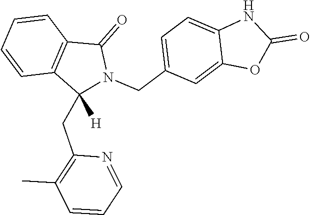 [SMILES: Cc1cccnc1C[C@@H]1c2ccccc2C(=O)N1Cc1ccc2[nH]c(=O)oc2c1] | 177 |
| 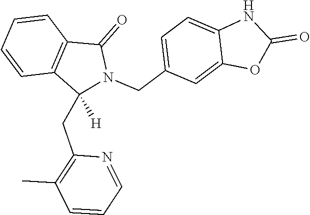 [SMILES: Cc1cccnc1C[C@H]1c2ccccc2C(=O)N1Cc1ccc2[nH]c(=O)oc2c1] | 178 |
| 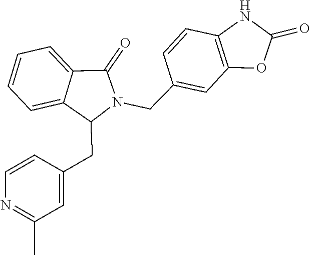 [SMILES: Cc1cc(CC2c3ccccc3C(=O)N2Cc2ccc3[nH]c(=O)oc3c2)ccn1] | 179 |
| 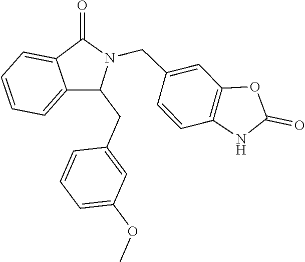 [SMILES: COc1cccc(CC2c3ccccc3C(=O)N2Cc2ccc3[nH]c(=O)oc3c2)c1] | 180 |
| 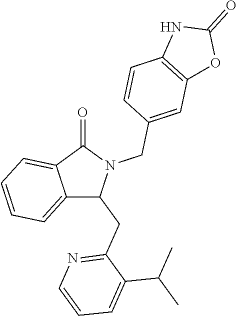 [SMILES: CC(C)c1cccnc1CC1c2ccccc2C(=O)N1Cc1ccc2[nH]c(=O)oc2c1] | 181 |
| 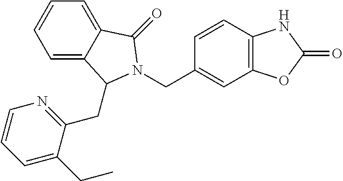 [SMILES: CCc1cccnc1CC1c2ccccc2C(=O)N1Cc1ccc2[nH]c(=O)oc2c1] | 182 |
| 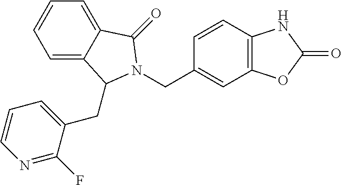 [SMILES: O=C1c2ccccc2C(Cc2cccnc2F)N1Cc1ccc2[nH]c(=O)oc2c1] | 183 |
| 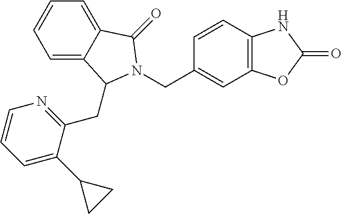 [SMILES: O=C1c2ccccc2C(Cc2ncccc2C2CC2)N1Cc1ccc2[nH]c(=O)oc2c1] | 184 |
| 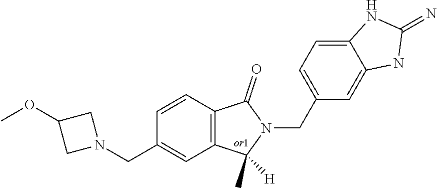 [SMILES: COC1CN(Cc2ccc3c(c2)[C@H](C)N(Cc2ccc4[nH]c(=N)[nH]c4c2)C3=O)C1] | 185 |
| 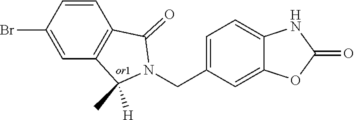 [SMILES: C[C@H]1c2cc(Br)ccc2C(=O)N1Cc1ccc2[nH]c(=O)oc2c1] | 186 |
| 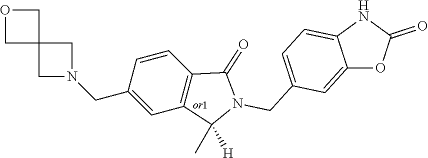 [SMILES: C[C@H]1c2cc(CN3CC4(COC4)C3)ccc2C(=O)N1Cc1ccc2[nH]c(=O)oc2c1] | 187 |
| 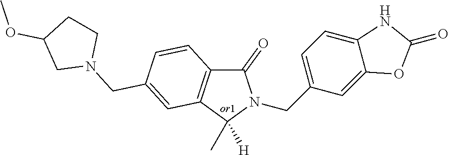 [SMILES: COC1CCN(Cc2ccc3c(c2)[C@H](C)N(Cc2ccc4[nH]c(=O)oc4c2)C3=O)C1] | 188 |
| 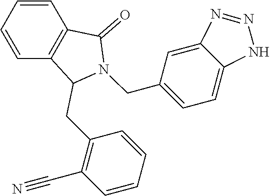 [SMILES: N#Cc1ccccc1CC1c2ccccc2C(=O)N1Cc1ccc2[nH]nnc2c1] | 189 |
| 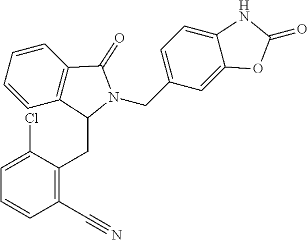 [SMILES: N#Cc1cccc(Cl)c1CC1c2ccccc2C(=O)N1Cc1ccc2[nH]c(=O)oc2c1] | 190 |
| 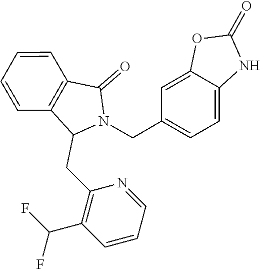 [SMILES: O=C1c2ccccc2C(Cc2ncccc2C(F)F)N1Cc1ccc2[nH]c(=O)oc2c1] | 191 |
| 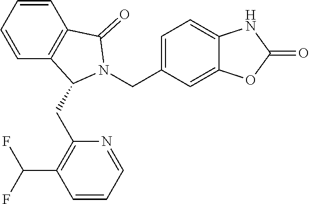 [SMILES: O=C1c2ccccc2[C@@H](Cc2ncccc2C(F)F)N1Cc1ccc2[nH]c(=O)oc2c1] | 192 |
| 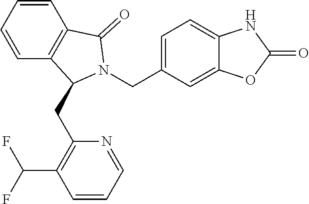 [SMILES: O=C1c2ccccc2[C@H](Cc2ncccc2C(F)F)N1Cc1ccc2[nH]c(=O)oc2c1] | 193 |
| 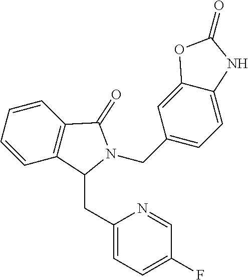 [SMILES: O=C1c2ccccc2C(Cc2ccc(F)cn2)N1Cc1ccc2[nH]c(=O)oc2c1] | 194 |
| 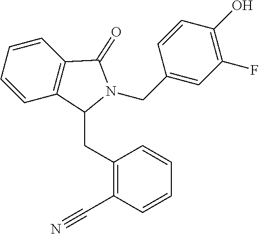 [SMILES: N#Cc1ccccc1CC1c2ccccc2C(=O)N1Cc1ccc(O)c(F)c1] | 195 |
| 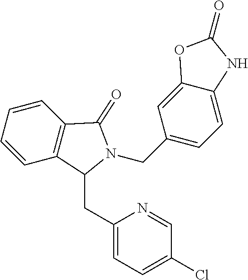 [SMILES: O=C1c2ccccc2C(Cc2ccc(Cl)cn2)N1Cc1ccc2[nH]c(=O)oc2c1] | 196 |
| 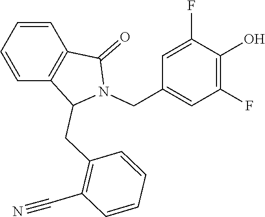 [SMILES: N#Cc1ccccc1CC1c2ccccc2C(=O)N1Cc1cc(F)c(O)c(F)c1] | 197 |
| 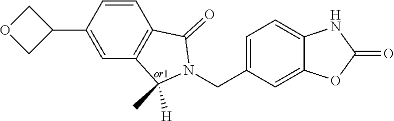 [SMILES: C[C@H]1c2cc(C3COC3)ccc2C(=O)N1Cc1ccc2[nH]c(=O)oc2c1] | 198 |
| 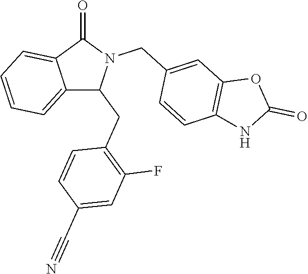 [SMILES: N#Cc1ccc(CC2c3ccccc3C(=O)N2Cc2ccc3[nH]c(=O)oc3c2)c(F)c1] | 199 |
| 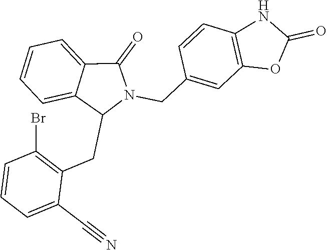 [SMILES: N#Cc1cccc(Br)c1CC1c2ccccc2C(=O)N1Cc1ccc2[nH]c(=O)oc2c1] | 200 |
| 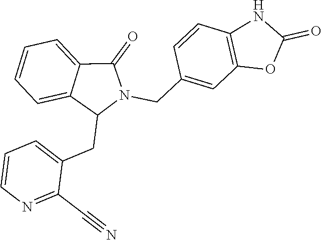 [SMILES: N#Cc1ncccc1CC1c2ccccc2C(=O)N1Cc1ccc2[nH]c(=O)oc2c1] | 201 |
| 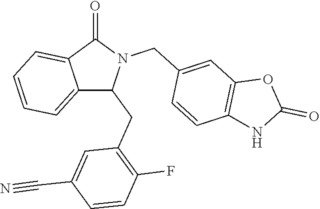 [SMILES: N#Cc1ccc(F)c(CC2c3ccccc3C(=O)N2Cc2ccc3[nH]c(=O)oc3c2)c1] | 202 |
| 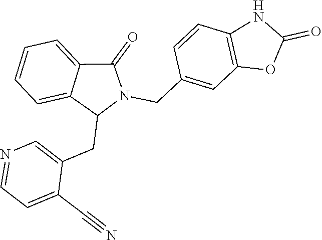 [SMILES: N#Cc1ccncc1CC1c2ccccc2C(=O)N1Cc1ccc2[nH]c(=O)oc2c1] | 203 |
| 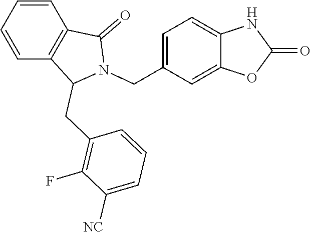 [SMILES: N#Cc1cccc(CC2c3ccccc3C(=O)N2Cc2ccc3[nH]c(=O)oc3c2)c1F] | 204 |
| 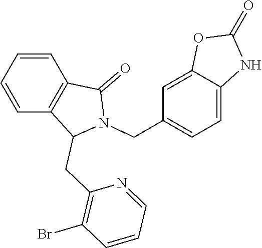 [SMILES: O=C1c2ccccc2C(Cc2ncccc2Br)N1Cc1ccc2[nH]c(=O)oc2c1] | 205 |
| 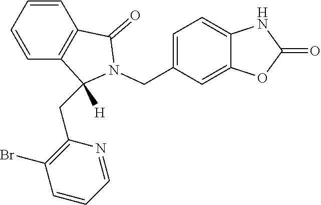 [SMILES: O=C1c2ccccc2[C@@H](Cc2ncccc2Br)N1Cc1ccc2[nH]c(=O)oc2c1] | 206 |
| 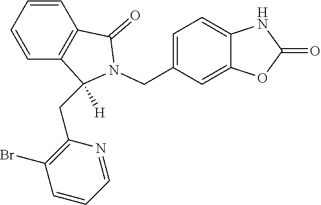 [SMILES: O=C1c2ccccc2[C@H](Cc2ncccc2Br)N1Cc1ccc2[nH]c(=O)oc2c1] | 207 |
| 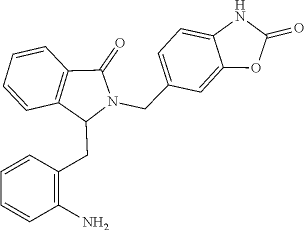 [SMILES: Nc1ccccc1CC1c2ccccc2C(=O)N1Cc1ccc2[nH]c(=O)oc2c1] | 208 |
| 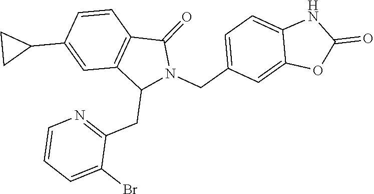 [SMILES: O=C1c2ccc(C3CC3)cc2C(Cc2ncccc2Br)N1Cc1ccc2[nH]c(=O)oc2c1] | 209 |
| 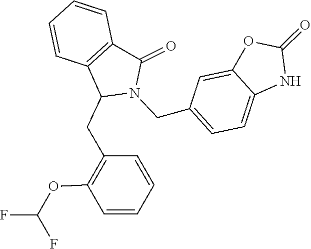 [SMILES: O=C1c2ccccc2C(Cc2ccccc2OC(F)F)N1Cc1ccc2[nH]c(=O)oc2c1] | 210 |
| 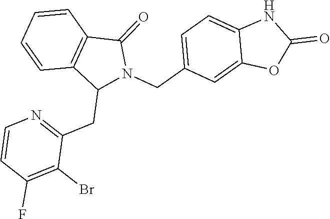 [SMILES: O=C1c2ccccc2C(Cc2nccc(F)c2Br)N1Cc1ccc2[nH]c(=O)oc2c1] | 211 |
| 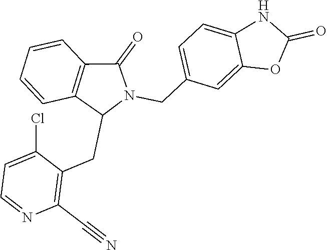 [SMILES: N#Cc1nccc(Cl)c1CC1c2ccccc2C(=O)N1Cc1ccc2[nH]c(=O)oc2c1] | 212 |
| 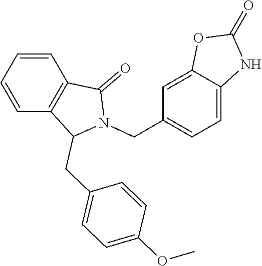 [SMILES: COc1ccc(CC2c3ccccc3C(=O)N2Cc2ccc3[nH]c(=O)oc3c2)cc1] | 213 |
| 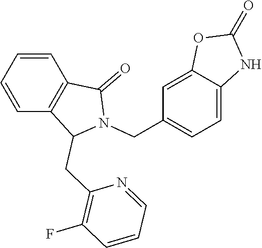 [SMILES: O=C1c2ccccc2C(Cc2ncccc2F)N1Cc1ccc2[nH]c(=O)oc2c1] | 214 |
| 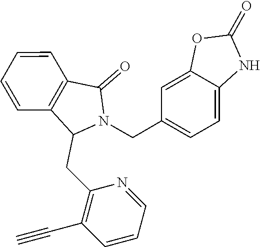 [SMILES: C#Cc1cccnc1CC1c2ccccc2C(=O)N1Cc1ccc2[nH]c(=O)oc2c1] | 215 |
| 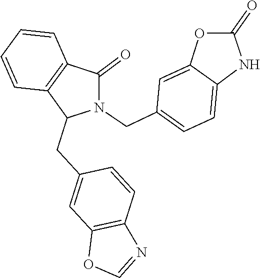 [SMILES: O=C1c2ccccc2C(Cc2ccc3ncoc3c2)N1Cc1ccc2[nH]c(=O)oc2c1] | 216 |
| 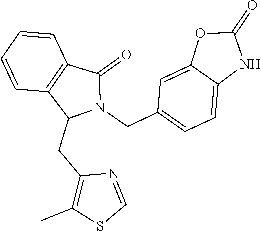 [SMILES: Cc1scnc1CC1c2ccccc2C(=O)N1Cc1ccc2[nH]c(=O)oc2c1] | 217 |
| 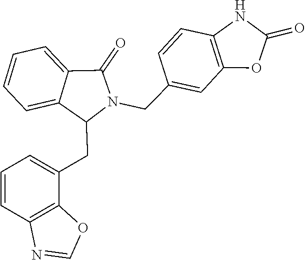 [SMILES: O=C1c2ccccc2C(Cc2cccc3ncoc23)N1Cc1ccc2[nH]c(=O)oc2c1] | 218 |
| 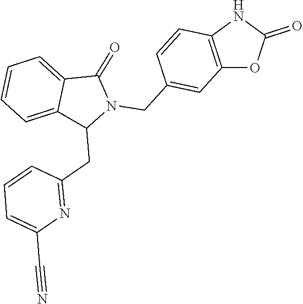 [SMILES: N#Cc1cccc(CC2c3ccccc3C(=O)N2Cc2ccc3[nH]c(=O)oc3c2)n1] | 219 |
| 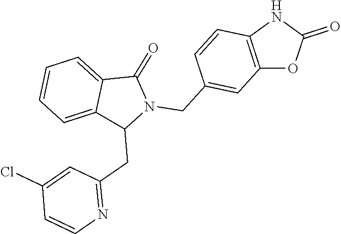 [SMILES: O=C1c2ccccc2C(Cc2cc(Cl)ccn2)N1Cc1ccc2[nH]c(=O)oc2c1] | 220 |
| 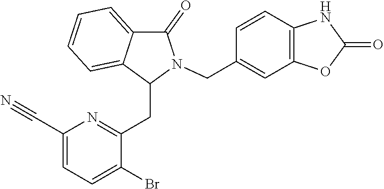 [SMILES: N#Cc1ccc(Br)c(CC2c3ccccc3C(=O)N2Cc2ccc3[nH]c(=O)oc3c2)n1] | 221 |
| 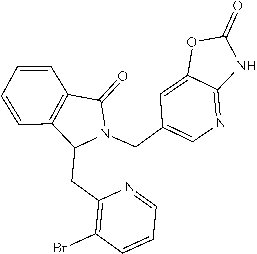 [SMILES: O=C1c2ccccc2C(Cc2ncccc2Br)N1Cc1cnc2[nH]c(=O)oc2c1] | 222 |
| 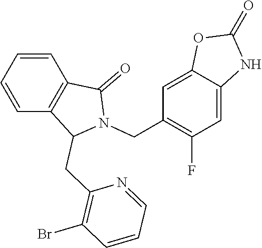 [SMILES: O=C1c2ccccc2C(Cc2ncccc2Br)N1Cc1cc2oc(=O)[nH]c2cc1F] | 223 |
| 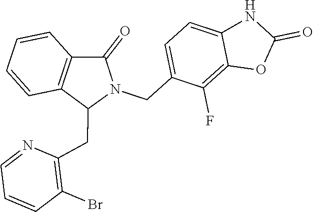 [SMILES: O=C1c2ccccc2C(Cc2ncccc2Br)N1Cc1ccc2[nH]c(=O)oc2c1F] | 224 |
| 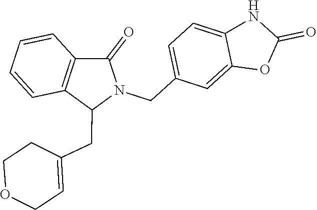 [SMILES: O=C1c2ccccc2C(CC2=CCOCC2)N1Cc1ccc2[nH]c(=O)oc2c1] | 225 |
| 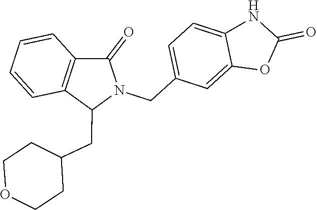 [SMILES: O=C1c2ccccc2C(CC2CCOCC2)N1Cc1ccc2[nH]c(=O)oc2c1] | 226 |
| 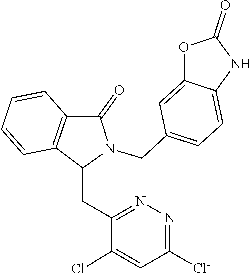 | 227 |
| 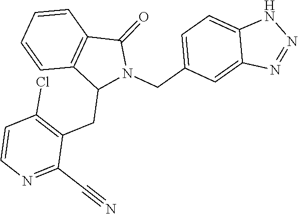 [SMILES: N#Cc1nccc(Cl)c1CC1c2ccccc2C(=O)N1Cc1ccc2[nH]nnc2c1] | 228 |
| 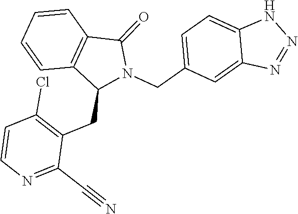 [SMILES: N#Cc1nccc(Cl)c1C[C@H]1c2ccccc2C(=O)N1Cc1ccc2[nH]nnc2c1] | 229 |
| 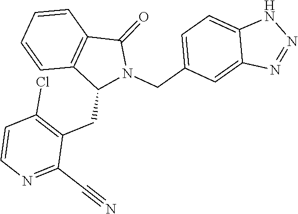 [SMILES: N#Cc1nccc(Cl)c1C[C@@H]1c2ccccc2C(=O)N1Cc1ccc2[nH]nnc2c1] | 230 |
| 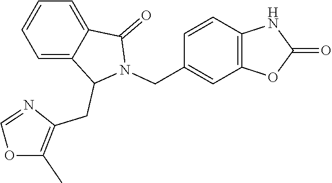 [SMILES: Cc1ocnc1CC1c2ccccc2C(=O)N1Cc1ccc2[nH]c(=O)oc2c1] | 231 |
| 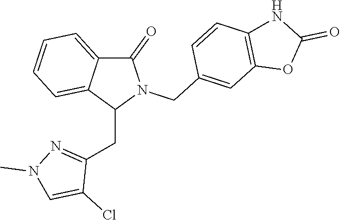 [SMILES: Cn1cc(Cl)c(CC2c3ccccc3C(=O)N2Cc2ccc3[nH]c(=O)oc3c2)n1] | 232 |
| 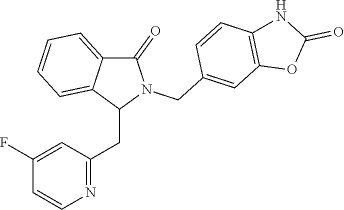 [SMILES: O=C1c2ccccc2C(Cc2cc(F)ccn2)N1Cc1ccc2[nH]c(=O)oc2c1] | 233 |
| 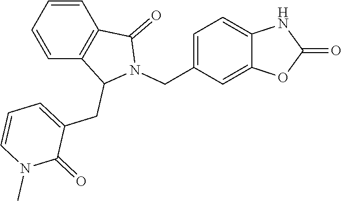 [SMILES: Cn1cccc(CC2c3ccccc3C(=O)N2Cc2ccc3[nH]c(=O)oc3c2)c1=O] | 234 |
| 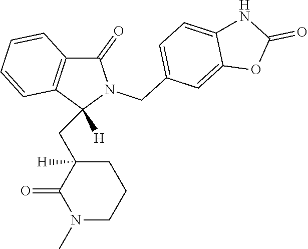 [SMILES: CN1CCC[C@H](C[C@@H]2c3ccccc3C(=O)N2Cc2ccc3[nH]c(=O)oc3c2)C1=O] | 235 |
| 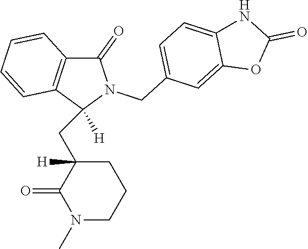 [SMILES: CN1CCC[C@@H](C[C@H]2c3ccccc3C(=O)N2Cc2ccc3[nH]c(=O)oc3c2)C1=O] | 236 |
| 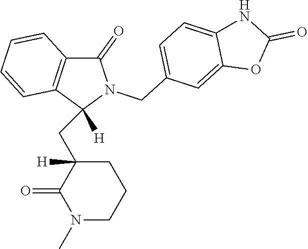 [SMILES: CN1CCC[C@@H](C[C@@H]2c3ccccc3C(=O)N2Cc2ccc3[nH]c(=O)oc3c2)C1=O] | 237 |
| 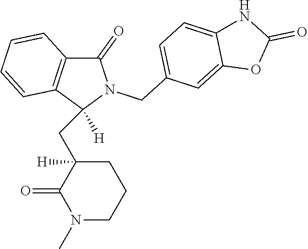 [SMILES: CN1CCC[C@H](C[C@H]2c3ccccc3C(=O)N2Cc2ccc3[nH]c(=O)oc3c2)C1=O] | 238 |
| 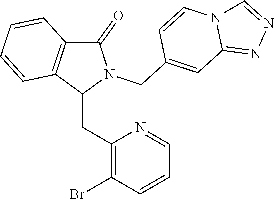 [SMILES: O=C1c2ccccc2C(Cc2ncccc2Br)N1Cc1ccn2cnnc2c1] | 239 |
| 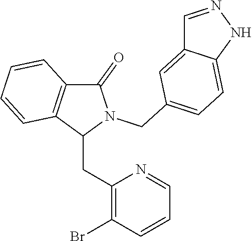 [SMILES: O=C1c2ccccc2C(Cc2ncccc2Br)N1Cc1ccc2[nH]ncc2c1] | 240 |
| 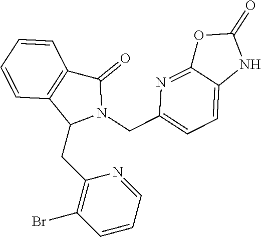 [SMILES: O=C1c2ccccc2C(Cc2ncccc2Br)N1Cc1ccc2[nH]c(=O)oc2n1] | 241 |
| 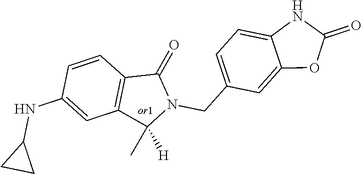 [SMILES: C[C@H]1c2cc(NC3CC3)ccc2C(=O)N1Cc1ccc2[nH]c(=O)oc2c1] | 242 |
| 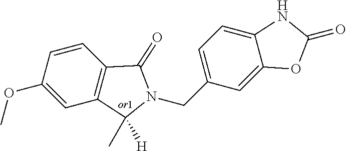 [SMILES: COc1ccc2c(c1)[C@H](C)N(Cc1ccc3[nH]c(=O)oc3c1)C2=O] | 243 |
| 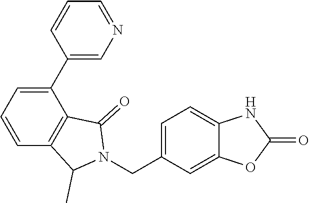 [SMILES: CC1c2cccc(-c3cccnc3)c2C(=O)N1Cc1ccc2[nH]c(=O)oc2c1] | 244 |
| 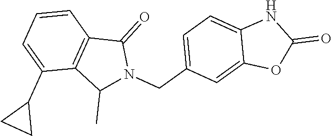 [SMILES: CC1c2c(cccc2C2CC2)C(=O)N1Cc1ccc2[nH]c(=O)oc2c1] | 245 |
| 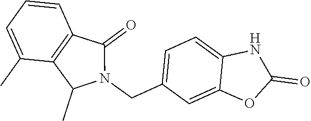 [SMILES: Cc1cccc2c1C(C)N(Cc1ccc3[nH]c(=O)oc3c1)C2=O] | 246 |
| 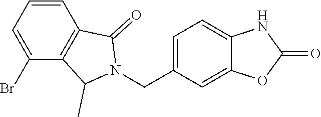 [SMILES: CC1c2c(Br)cccc2C(=O)N1Cc1ccc2[nH]c(=O)oc2c1] | 247 |
| 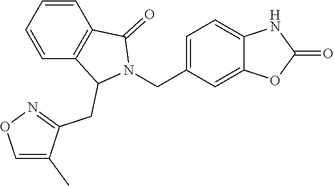 [SMILES: Cc1conc1CC1c2ccccc2C(=O)N1Cc1ccc2[nH]c(=O)oc2c1] | 248 |
| 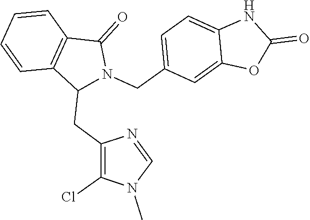 [SMILES: Cn1cnc(CC2c3ccccc3C(=O)N2Cc2ccc3[nH]c(=O)oc3c2)c1Cl] | 249 |
| 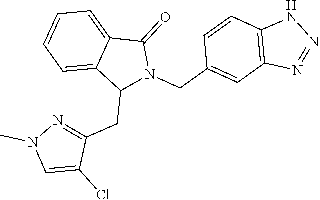 [SMILES: Cn1cc(Cl)c(CC2c3ccccc3C(=O)N2Cc2ccc3[nH]nnc3c2)n1] | 250 |
| 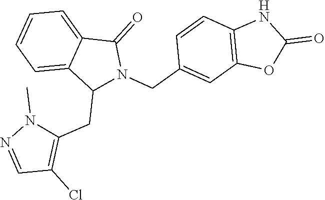 [SMILES: Cn1ncc(Cl)c1CC1c2ccccc2C(=O)N1Cc1ccc2[nH]c(=O)oc2c1] | 251 |
| 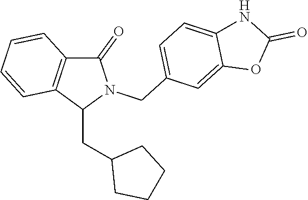 [SMILES: O=C1c2ccccc2C(CC2CCCC2)N1Cc1ccc2[nH]c(=O)oc2c1] | 252 |
| 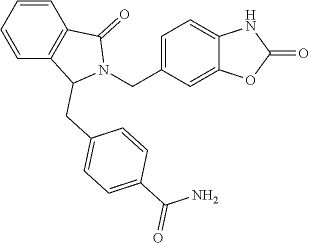 [SMILES: NC(=O)c1ccc(CC2c3ccccc3C(=O)N2Cc2ccc3[nH]c(=O)oc3c2)cc1] | 253 |
| 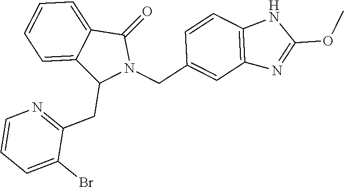 [SMILES: COc1nc2cc(CN3C(=O)c4ccccc4C3Cc3ncccc3Br)ccc2[nH]1] | 254 |
| 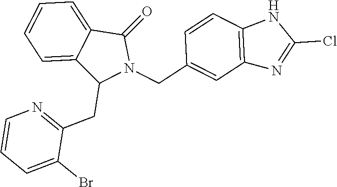 [SMILES: O=C1c2ccccc2C(Cc2ncccc2Br)N1Cc1ccc2[nH]c(Cl)nc2c1] | 255 |
| 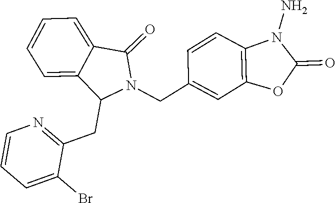 [SMILES: Nn1c(=O)oc2cc(CN3C(=O)c4ccccc4C3Cc3ncccc3Br)ccc21] | 256 |
| 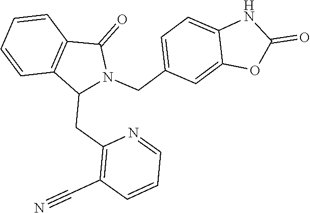 [SMILES: N#Cc1cccnc1CC1c2ccccc2C(=O)N1Cc1ccc2[nH]c(=O)oc2c1] | 257 |
| 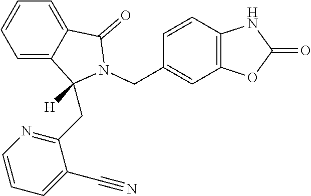 [SMILES: N#Cc1cccnc1C[C@@H]1c2ccccc2C(=O)N1Cc1ccc2[nH]c(=O)oc2c1] | 258 |
| 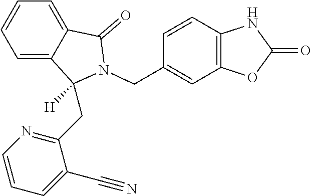 [SMILES: N#Cc1cccnc1C[C@H]1c2ccccc2C(=O)N1Cc1ccc2[nH]c(=O)oc2c1] | 259 |
| 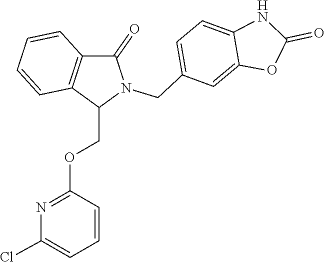 [SMILES: O=C1c2ccccc2C(COc2cccc(Cl)n2)N1Cc1ccc2[nH]c(=O)oc2c1] | 260 |
| 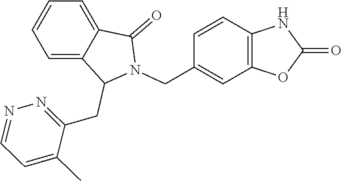 [SMILES: Cc1ccnnc1CC1c2ccccc2C(=O)N1Cc1ccc2[nH]c(=O)oc2c1] | 261 |
| 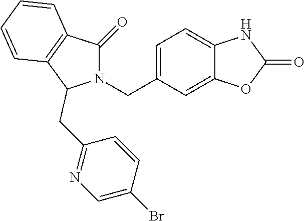 [SMILES: O=C1c2ccccc2C(Cc2ccc(Br)cn2)N1Cc1ccc2[nH]c(=O)oc2c1] | 262 |
| 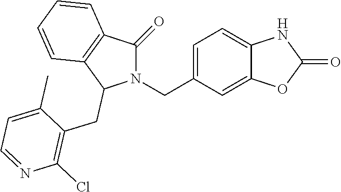 [SMILES: Cc1ccnc(Cl)c1CC1c2ccccc2C(=O)N1Cc1ccc2[nH]c(=O)oc2c1] | 263 |
| 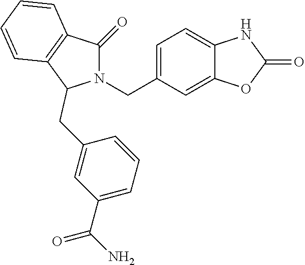 [SMILES: NC(=O)c1cccc(CC2c3ccccc3C(=O)N2Cc2ccc3[nH]c(=O)oc3c2)c1] | 264 |
| 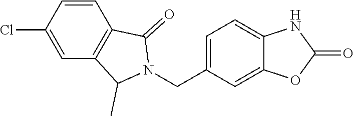 [SMILES: CC1c2cc(Cl)ccc2C(=O)N1Cc1ccc2[nH]c(=O)oc2c1] | 265 |
| 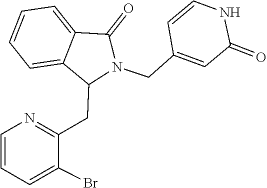 [SMILES: O=C1c2ccccc2C(Cc2ncccc2Br)N1Cc1cc[nH]c(=O)c1] | 266 |
| 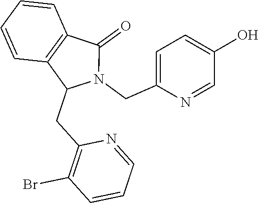 [SMILES: O=C1c2ccccc2C(Cc2ncccc2Br)N1Cc1ccc(O)cn1] | 267 |
| 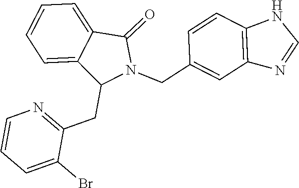 [SMILES: O=C1c2ccccc2C(Cc2ncccc2Br)N1Cc1ccc2[nH]cnc2c1] | 268 |
| 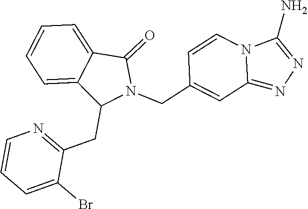 [SMILES: Nc1nnc2cc(CN3C(=O)c4ccccc4C3Cc3ncccc3Br)ccn12] | 269 |
| 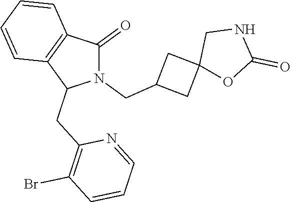 [SMILES: O=C1NCC2(CC(CN3C(=O)c4ccccc4C3Cc3ncccc3Br)C2)O1] | 270 |
| 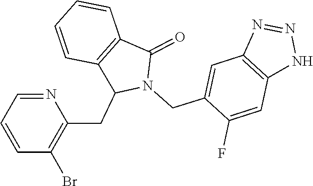 [SMILES: O=C1c2ccccc2C(Cc2ncccc2Br)N1Cc1cc2nn[nH]c2cc1F] | 271 |
| 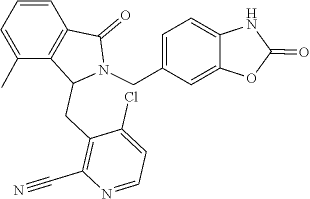 [SMILES: Cc1cccc2c1C(Cc1c(Cl)ccnc1C#N)N(Cc1ccc3[nH]c(=O)oc3c1)C2=O] | 272 |
| 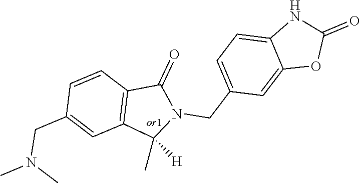 [SMILES: C[C@H]1c2cc(CN(C)C)ccc2C(=O)N1Cc1ccc2[nH]c(=O)oc2c1] | 273 |
| 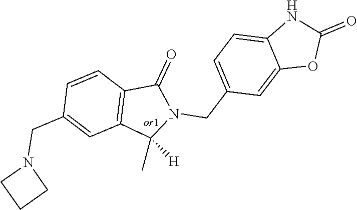 [SMILES: C[C@H]1c2cc(CN3CCC3)ccc2C(=O)N1Cc1ccc2[nH]c(=O)oc2c1] | 274 |
| 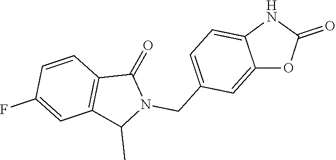 [SMILES: CC1c2cc(F)ccc2C(=O)N1Cc1ccc2[nH]c(=O)oc2c1] | 275 |
| 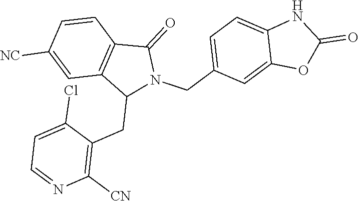 [SMILES: N#Cc1ccc2c(c1)C(Cc1c(Cl)ccnc1C#N)N(Cc1ccc3[nH]c(=O)oc3c1)C2=O] | 276 |
| 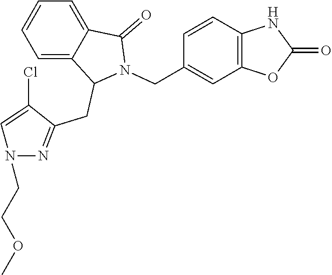 [SMILES: COCCn1cc(Cl)c(CC2c3ccccc3C(=O)N2Cc2ccc3[nH]c(=O)oc3c2)n1] | 277 |
| 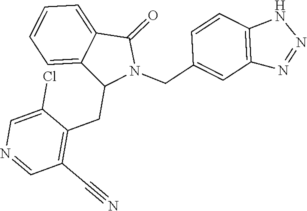 [SMILES: N#Cc1cncc(Cl)c1CC1c2ccccc2C(=O)N1Cc1ccc2[nH]nnc2c1] | 278 |
| 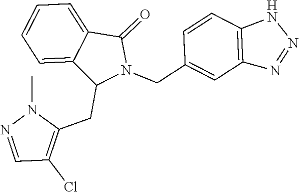 [SMILES: Cn1ncc(Cl)c1CC1c2ccccc2C(=O)N1Cc1ccc2[nH]nnc2c1] | 279 |
| 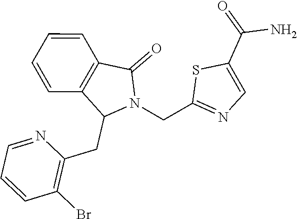 [SMILES: NC(=O)c1cnc(CN2C(=O)c3ccccc3C2Cc2ncccc2Br)s1] | 280 |
| 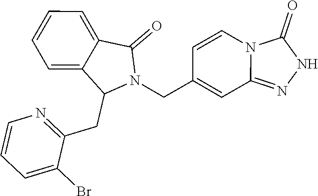 [SMILES: O=C1c2ccccc2C(Cc2ncccc2Br)N1Cc1ccn2c(=O)[nH]nc2c1] | 281 |
| 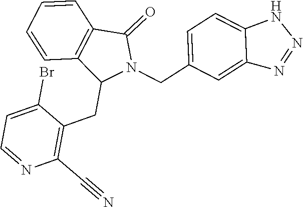 [SMILES: N#Cc1nccc(Br)c1CC1c2ccccc2C(=O)N1Cc1ccc2[nH]nnc2c1] | 282 |
| 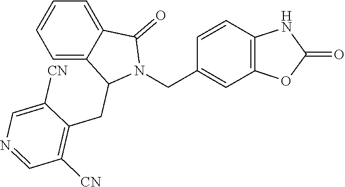 [SMILES: N#Cc1cncc(C#N)c1CC1c2ccccc2C(=O)N1Cc1ccc2[nH]c(=O)oc2c1] | 283 |
| 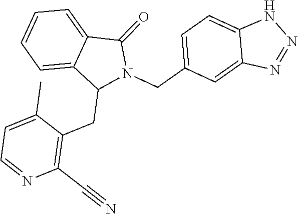 [SMILES: Cc1ccnc(C#N)c1CC1c2ccccc2C(=O)N1Cc1ccc2[nH]nnc2c1] | 284 |
| 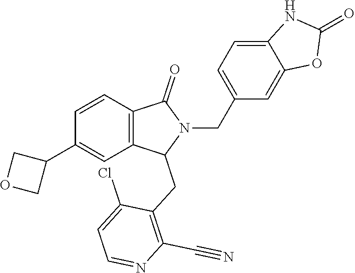 [SMILES: N#Cc1nccc(Cl)c1CC1c2cc(C3COC3)ccc2C(=O)N1Cc1ccc2[nH]c(=O)oc2c1] | 285 |
| 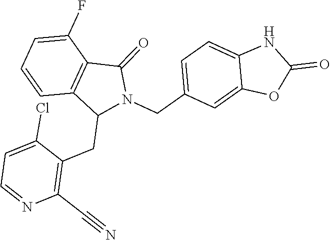 [SMILES: N#Cc1nccc(Cl)c1CC1c2cccc(F)c2C(=O)N1Cc1ccc2[nH]c(=O)oc2c1] | 286 |
| 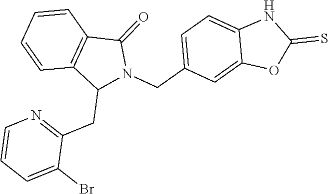 [SMILES: O=C1c2ccccc2C(Cc2ncccc2Br)N1Cc1ccc2[nH]c(=S)oc2c1] | 287 |
| 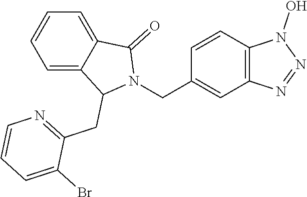 [SMILES: O=C1c2ccccc2C(Cc2ncccc2Br)N1Cc1ccc2c(c1)nnn2O] | 288 |
| 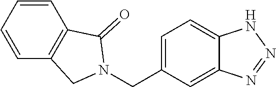 [SMILES: O=C1c2ccccc2CN1Cc1ccc2[nH]nnc2c1] | 289 |
| 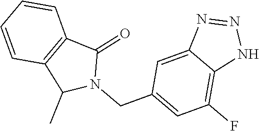 [SMILES: CC1c2ccccc2C(=O)N1Cc1cc(F)c2[nH]nnc2c1] | 290 |
| 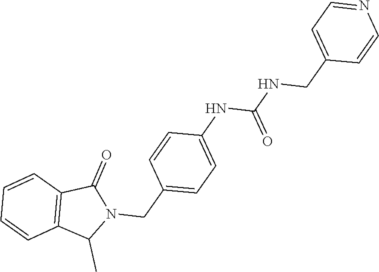 [SMILES: CC1c2ccccc2C(=O)N1Cc1ccc(NC(=O)NCc2ccncc2)cc1] | 291 |
| 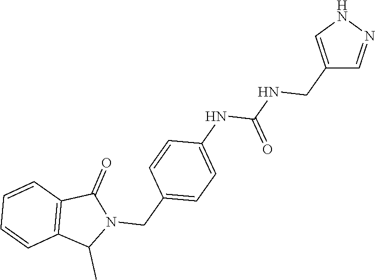 [SMILES: CC1c2ccccc2C(=O)N1Cc1ccc(NC(=O)NCc2cn[nH]c2)cc1] | 292 |
| 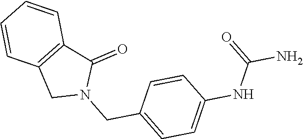 [SMILES: NC(=O)Nc1ccc(CN2Cc3ccccc3C2=O)cc1] | 293 |
| 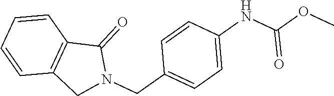 [SMILES: COC(=O)Nc1ccc(CN2Cc3ccccc3C2=O)cc1] | 294 |
| 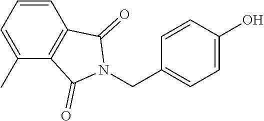 [SMILES: Cc1cccc2c1C(=O)N(Cc1ccc(O)cc1)C2=O] | 295 |
| 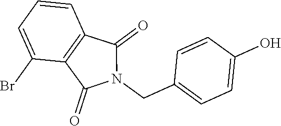 [SMILES: O=C1c2cccc(Br)c2C(=O)N1Cc1ccc(O)cc1] | 296 |
| 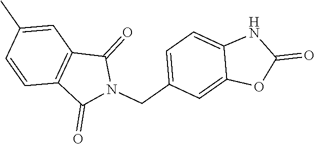 [SMILES: Cc1ccc2c(c1)C(=O)N(Cc1ccc3[nH]c(=O)oc3c1)C2=O] | 297 |
| 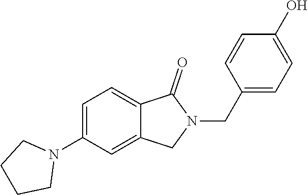 [SMILES: O=C1c2ccc(N3CCCC3)cc2CN1Cc1ccc(O)cc1] | 298 |
| 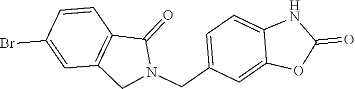 [SMILES: O=C1c2ccc(Br)cc2CN1Cc1ccc2[nH]c(=O)oc2c1] | 299 |
| 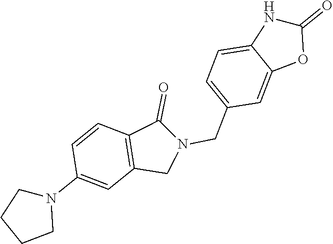 [SMILES: O=C1c2ccc(N3CCCC3)cc2CN1Cc1ccc2[nH]c(=O)oc2c1] | 300 |
| 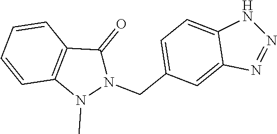 [SMILES: Cn1c2ccccc2c(=O)n1Cc1ccc2[nH]nnc2c1] | 301 |
| 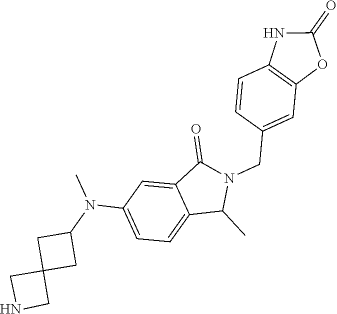 [SMILES: CC1c2ccc(N(C)C3CC4(CNC4)C3)cc2C(=O)N1Cc1ccc2[nH]c(=O)oc2c1] | 302 |
| 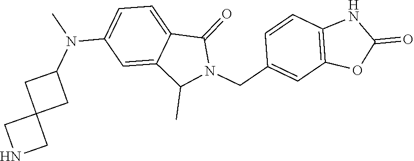 [SMILES: CC1c2cc(N(C)C3CC4(CNC4)C3)ccc2C(=O)N1Cc1ccc2[nH]c(=O)oc2c1] | 303 |
| 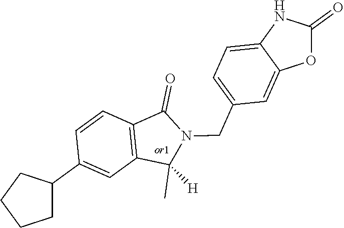 [SMILES: C[C@H]1c2cc(C3CCCC3)ccc2C(=O)N1Cc1ccc2[nH]c(=O)oc2c1] | 304 |
| 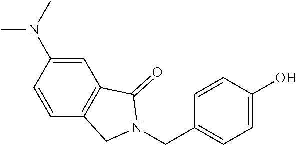 [SMILES: CN(C)c1ccc2c(c1)C(=O)N(Cc1ccc(O)cc1)C2] | 305 |
| 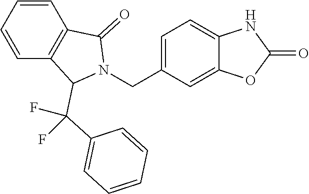 [SMILES: O=C1c2ccccc2C(C(F)(F)c2ccccc2)N1Cc1ccc2[nH]c(=O)oc2c1] | 306 |
| 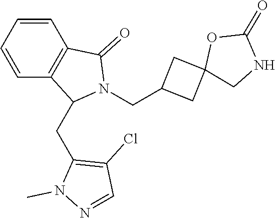 [SMILES: Cn1ncc(Cl)c1CC1c2ccccc2C(=O)N1CC1CC2(CNC(=O)O2)C1] | 307 |
| 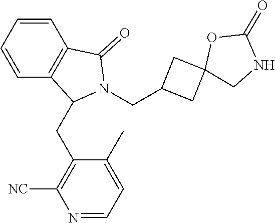 [SMILES: Cc1ccnc(C#N)c1CC1c2ccccc2C(=O)N1CC1CC2(CNC(=O)O2)C1] | 308 |
| 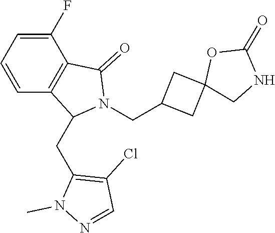 [SMILES: Cn1ncc(Cl)c1CC1c2cccc(F)c2C(=O)N1CC1CC2(CNC(=O)O2)C1] | 309 |
| 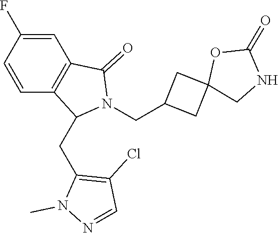 [SMILES: Cn1ncc(Cl)c1CC1c2ccc(F)cc2C(=O)N1CC1CC2(CNC(=O)O2)C1] | 310 |
| 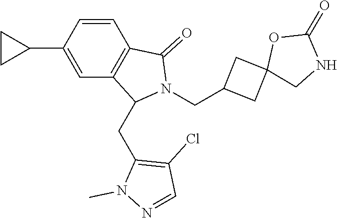 [SMILES: Cn1ncc(Cl)c1CC1c2cc(C3CC3)ccc2C(=O)N1CC1CC2(CNC(=O)O2)C1] | 311 |
| 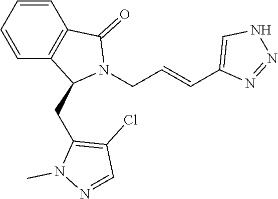 [SMILES: Cn1ncc(Cl)c1C[C@H]1c2ccccc2C(=O)N1C/C=C/c1c[nH]nn1] | 312 |
| 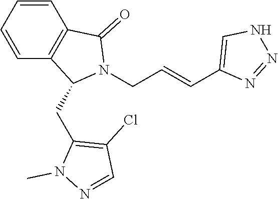 [SMILES: Cn1ncc(Cl)c1C[C@@H]1c2ccccc2C(=O)N1C/C=C/c1c[nH]nn1] | 313 |
| 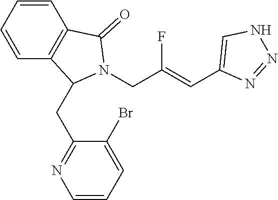 [SMILES: O=C1c2ccccc2C(Cc2ncccc2Br)N1C/C(F)=C/c1c[nH]nn1] | 314 |
| 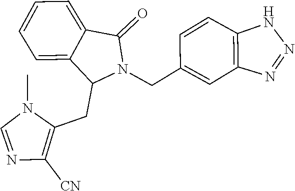 [SMILES: Cn1cnc(C#N)c1CC1c2ccccc2C(=O)N1Cc1ccc2[nH]nnc2c1] | 315 |
| 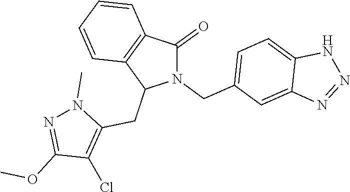 [SMILES: COc1nn(C)c(CC2c3ccccc3C(=O)N2Cc2ccc3[nH]nnc3c2)c1Cl] | 316 |
| 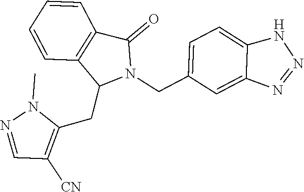 [SMILES: Cn1ncc(C#N)c1CC1c2ccccc2C(=O)N1Cc1ccc2[nH]nnc2c1] | 317 |
| 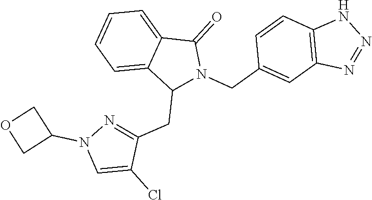 [SMILES: O=C1c2ccccc2C(Cc2nn(C3COC3)cc2Cl)N1Cc1ccc2[nH]nnc2c1] | 318 |
| 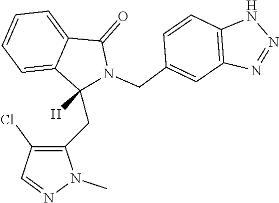 [SMILES: Cn1ncc(Cl)c1C[C@@H]1c2ccccc2C(=O)N1Cc1ccc2[nH]nnc2c1] | 319 |
| 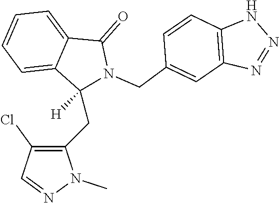 [SMILES: Cn1ncc(Cl)c1C[C@H]1c2ccccc2C(=O)N1Cc1ccc2[nH]nnc2c1] | 320 |
| 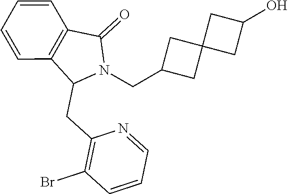 [SMILES: O=C1c2ccccc2C(Cc2ncccc2Br)N1CC1CC2(CC(O)C2)C1] | 321 |
| 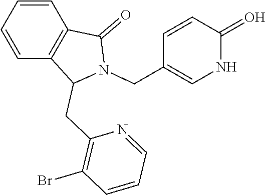 [SMILES: O=C1c2ccccc2C(Cc2ncccc2Br)N1Cc1ccc(=O)[nH]c1] | 322 |
| 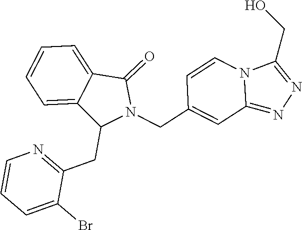 [SMILES: O=C1c2ccccc2C(Cc2ncccc2Br)N1Cc1ccn2c(CO)nnc2c1] | 323 |
| 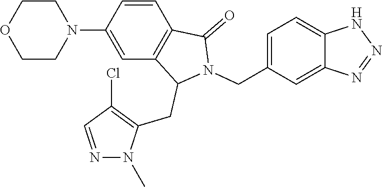 [SMILES: Cn1ncc(Cl)c1CC1c2cc(N3CCOCC3)ccc2C(=O)N1Cc1ccc2[nH]nnc2c1] | 324 |
| 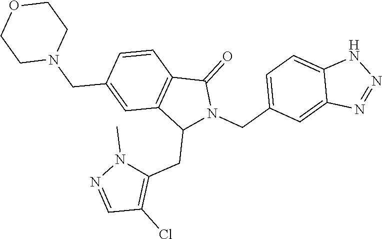 [SMILES: Cn1ncc(Cl)c1CC1c2cc(CN3CCOCC3)ccc2C(=O)N1Cc1ccc2[nH]nnc2c1] | 325 |
| 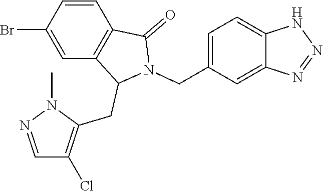 [SMILES: Cn1ncc(Cl)c1CC1c2cc(Br)ccc2C(=O)N1Cc1ccc2[nH]nnc2c1] | 326 |
| 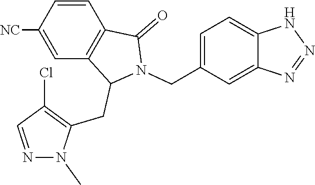 | 327 |
| 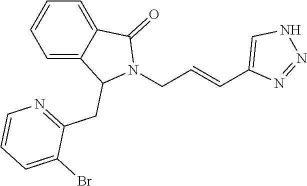 [SMILES: O=C1c2ccccc2C(Cc2ncccc2Br)N1C/C=C/c1c[nH]nn1] | 328 |
| 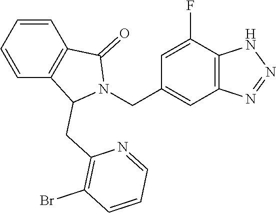 [SMILES: O=C1c2ccccc2C(Cc2ncccc2Br)N1Cc1cc(F)c2[nH]nnc2c1] | 329 |
| 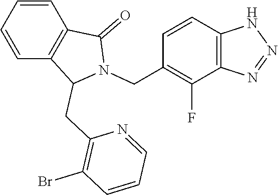 [SMILES: O=C1c2ccccc2C(Cc2ncccc2Br)N1Cc1ccc2[nH]nnc2c1F] | 330 |
| 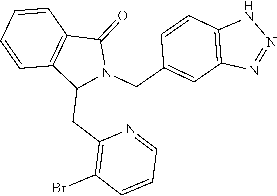 [SMILES: O=C1c2ccccc2C(Cc2ncccc2Br)N1Cc1ccc2[nH]nnc2c1] | 331 |
| 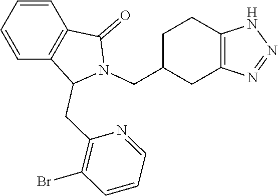 [SMILES: O=C1c2ccccc2C(Cc2ncccc2Br)N1CC1CCc2[nH]nnc2C1] | 332 |
| 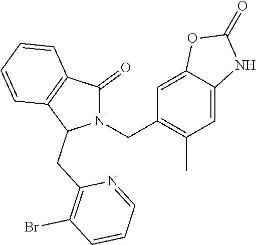 [SMILES: Cc1cc2[nH]c(=O)oc2cc1CN1C(=O)c2ccccc2C1Cc1ncccc1Br] | 333 |
| 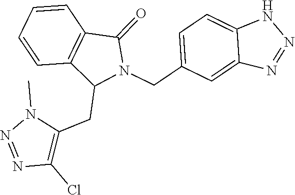 [SMILES: Cn1nnc(Cl)c1CC1c2ccccc2C(=O)N1Cc1ccc2[nH]nnc2c1] | 334 |
| 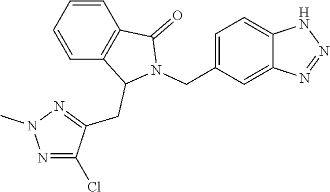 [SMILES: Cn1nc(Cl)c(CC2c3ccccc3C(=O)N2Cc2ccc3[nH]nnc3c2)n1] | 335 |
| 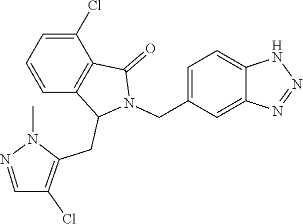 [SMILES: Cn1ncc(Cl)c1CC1c2cccc(Cl)c2C(=O)N1Cc1ccc2[nH]nnc2c1] | 336 |
| 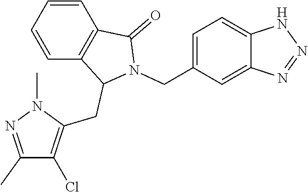 [SMILES: Cc1nn(C)c(CC2c3ccccc3C(=O)N2Cc2ccc3[nH]nnc3c2)c1Cl] | 337 |
| 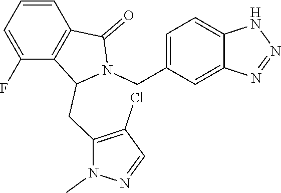 [SMILES: Cn1ncc(Cl)c1CC1c2c(F)cccc2C(=O)N1Cc1ccc2[nH]nnc2c1] | 338 |
| 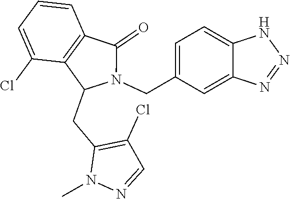 [SMILES: Cn1ncc(Cl)c1CC1c2c(Cl)cccc2C(=O)N1Cc1ccc2[nH]nnc2c1] | 339 |
| 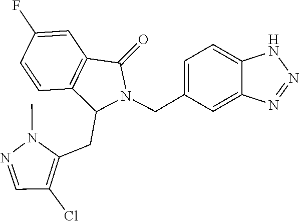 [SMILES: Cn1ncc(Cl)c1CC1c2ccc(F)cc2C(=O)N1Cc1ccc2[nH]nnc2c1] | 340 |
| 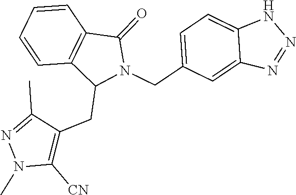 [SMILES: Cc1nn(C)c(C#N)c1CC1c2ccccc2C(=O)N1Cc1ccc2[nH]nnc2c1] | 341 |
| 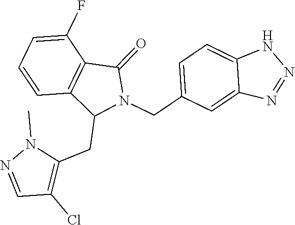 [SMILES: Cn1ncc(Cl)c1CC1c2cccc(F)c2C(=O)N1Cc1ccc2[nH]nnc2c1] | 342 |
| 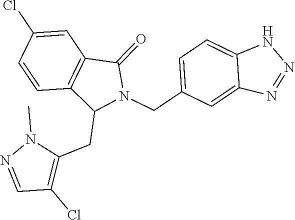 [SMILES: Cn1ncc(Cl)c1CC1c2ccc(Cl)cc2C(=O)N1Cc1ccc2[nH]nnc2c1] | 343 |
| 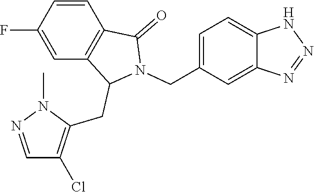 [SMILES: Cn1ncc(Cl)c1CC1c2cc(F)ccc2C(=O)N1Cc1ccc2[nH]nnc2c1] | 344 |
| 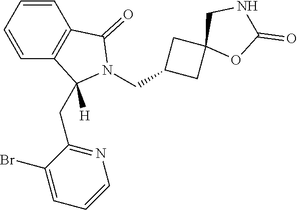 [SMILES: O=C1NC[C@]2(C[C@H](CN3C(=O)c4ccccc4[C@H]3Cc3ncccc3Br)C2)O1] | 345 |
| 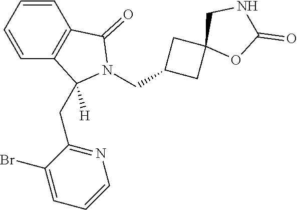 [SMILES: O=C1NC[C@]2(C[C@H](CN3C(=O)c4ccccc4[C@@H]3Cc3ncccc3Br)C2)O1] | 346 |
| 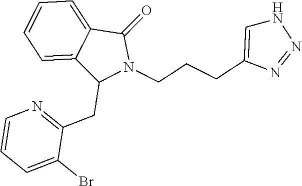 [SMILES: O=C1c2ccccc2C(Cc2ncccc2Br)N1CCCc1c[nH]nn1] | 347 |
| 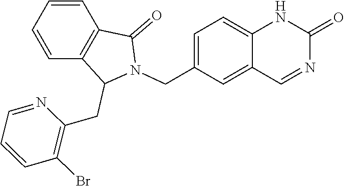 [SMILES: O=C1c2ccccc2C(Cc2ncccc2Br)N1Cc1ccc2[nH]c(=O)ncc2c1] | 348 |
| 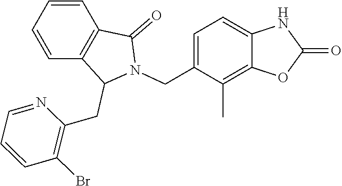 [SMILES: Cc1c(CN2C(=O)c3ccccc3C2Cc2ncccc2Br)ccc2[nH]c(=O)oc12] | 349 |
| 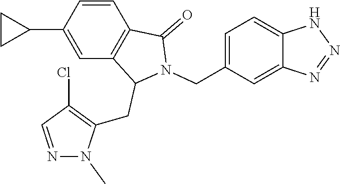 [SMILES: Cn1ncc(Cl)c1CC1c2cc(C3CC3)ccc2C(=O)N1Cc1ccc2[nH]nnc2c1] | 350 |
| 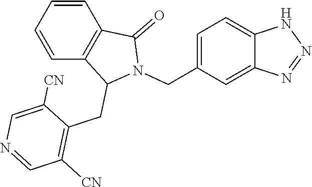 [SMILES: N#Cc1cncc(C#N)c1CC1c2ccccc2C(=O)N1Cc1ccc2[nH]nnc2c1] | 351 |
| 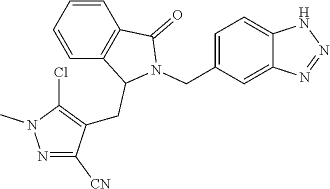 [SMILES: Cn1nc(C#N)c(CC2c3ccccc3C(=O)N2Cc2ccc3[nH]nnc3c2)c1Cl] | 352 |
| 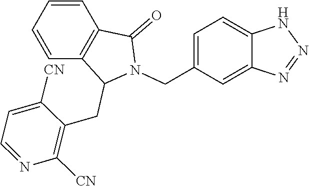 [SMILES: N#Cc1ccnc(C#N)c1CC1c2ccccc2C(=O)N1Cc1ccc2[nH]nnc2c1] | 353 |
| 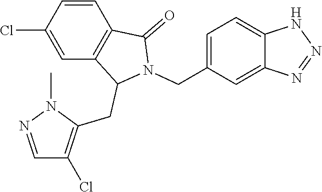 [SMILES: Cn1ncc(Cl)c1CC1c2cc(Cl)ccc2C(=O)N1Cc1ccc2[nH]nnc2c1] | 354 |
| 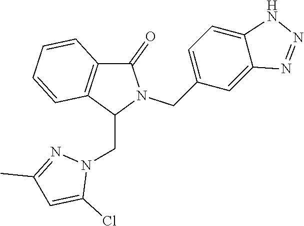 [SMILES: Cc1cc(Cl)n(CC2c3ccccc3C(=O)N2Cc2ccc3[nH]nnc3c2)n1] | 355 |
| 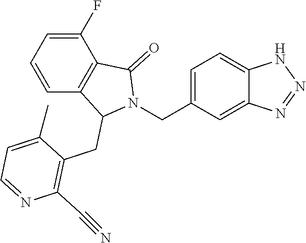 [SMILES: Cc1ccnc(C#N)c1CC1c2cccc(F)c2C(=O)N1Cc1ccc2[nH]nnc2c1] | 356 |
| 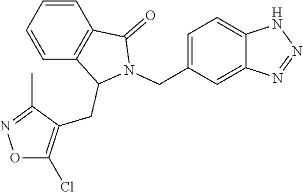 [SMILES: Cc1noc(Cl)c1CC1c2ccccc2C(=O)N1Cc1ccc2[nH]nnc2c1] | 357 |
| 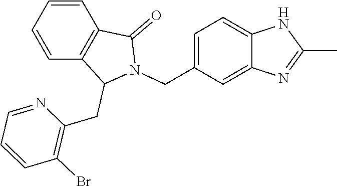 [SMILES: Cc1nc2cc(CN3C(=O)c4ccccc4C3Cc3ncccc3Br)ccc2[nH]1] | 358 |
| 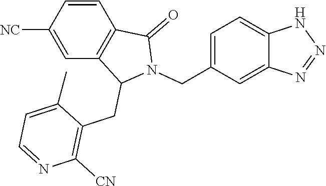 [SMILES: Cc1ccnc(C#N)c1CC1c2cc(C#N)ccc2C(=O)N1Cc1ccc2[nH]nnc2c1] | 359 |
| 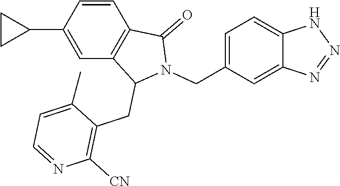 [SMILES: Cc1ccnc(C#N)c1CC1c2cc(C3CC3)ccc2C(=O)N1Cc1ccc2[nH]nnc2c1] | 360 |
| 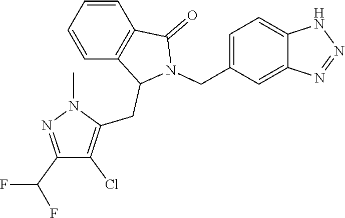 [SMILES: Cn1nc(C(F)F)c(Cl)c1CC1c2ccccc2C(=O)N1Cc1ccc2[nH]nnc2c1] | 361 |
| 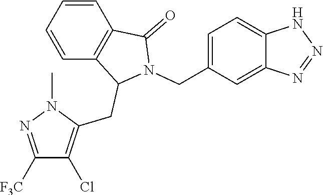 [SMILES: Cn1nc(C(F)(F)F)c(Cl)c1CC1c2ccccc2C(=O)N1Cc1ccc2[nH]nnc2c1] | 362 |
| 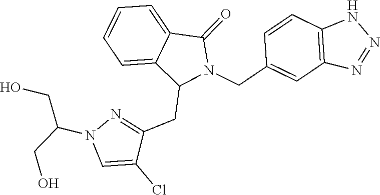 [SMILES: O=C1c2ccccc2C(Cc2nn(C(CO)CO)cc2Cl)N1Cc1ccc2[nH]nnc2c1] | 363 |
| 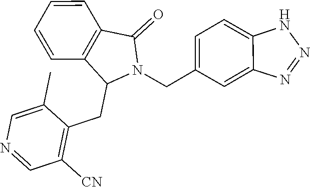 [SMILES: Cc1cncc(C#N)c1CC1c2ccccc2C(=O)N1Cc1ccc2[nH]nnc2c1] | 364 |
| 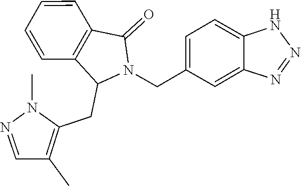 [SMILES: Cc1cnn(C)c1CC1c2ccccc2C(=O)N1Cc1ccc2[nH]nnc2c1] | 365 |
| 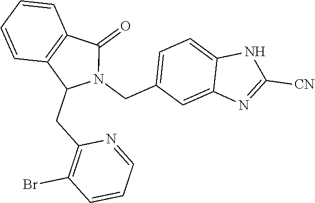 [SMILES: N#Cc1nc2cc(CN3C(=O)c4ccccc4C3Cc3ncccc3Br)ccc2[nH]1] | 366 |
| 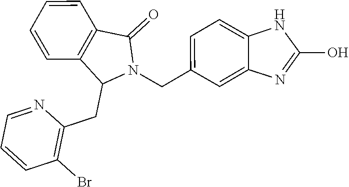 [SMILES: O=C1c2ccccc2C(Cc2ncccc2Br)N1Cc1ccc2[nH]c(O)nc2c1] | 367 |
| 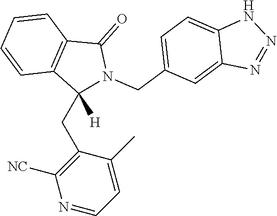 [SMILES: Cc1ccnc(C#N)c1C[C@@H]1c2ccccc2C(=O)N1Cc1ccc2[nH]nnc2c1] | 368 |
| 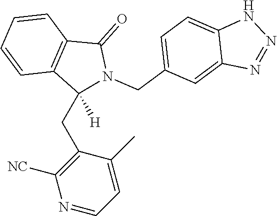 [SMILES: Cc1ccnc(C#N)c1C[C@H]1c2ccccc2C(=O)N1Cc1ccc2[nH]nnc2c1] | 369 |
| 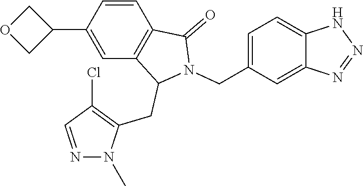 [SMILES: Cn1ncc(Cl)c1CC1c2cc(C3COC3)ccc2C(=O)N1Cc1ccc2[nH]nnc2c1] | 370 |
| 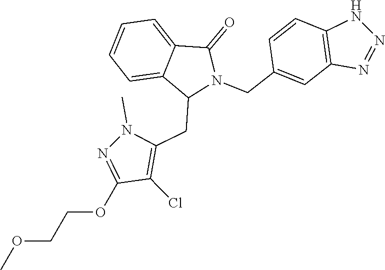 [SMILES: COCCOc1nn(C)c(CC2c3ccccc3C(=O)N2Cc2ccc3[nH]nnc3c2)c1Cl] | 371 |
| 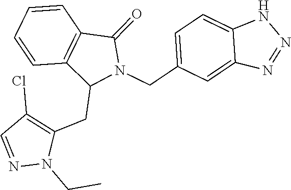 [SMILES: CCn1ncc(Cl)c1CC1c2ccccc2C(=O)N1Cc1ccc2[nH]nnc2c1] | 372 |
| 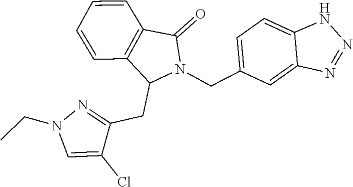 [SMILES: CCn1cc(Cl)c(CC2c3ccccc3C(=O)N2Cc2ccc3[nH]nnc3c2)n1] | 373 |
| 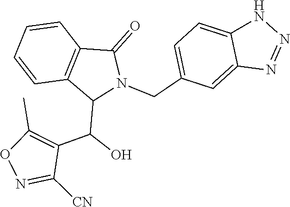 [SMILES: Cc1onc(C#N)c1C(O)C1c2ccccc2C(=O)N1Cc1ccc2[nH]nnc2c1] | 374 |
| 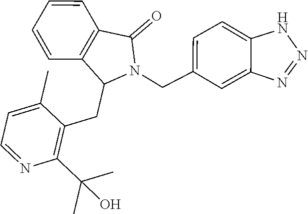 [SMILES: Cc1ccnc(C(C)(C)O)c1CC1c2ccccc2C(=O)N1Cc1ccc2[nH]nnc2c1] | 375 |
| 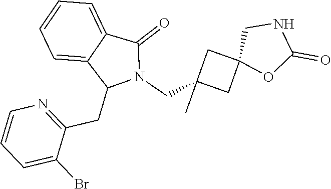 [SMILES: C[C@]1(CN2C(=O)c3ccccc3C2Cc2ncccc2Br)C[C@@]2(CNC(=O)O2)C1] | 376 |
| 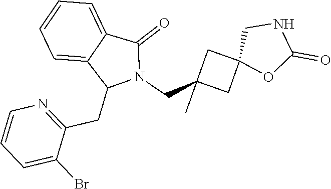 [SMILES: C[C@]1(CN2C(=O)c3ccccc3C2Cc2ncccc2Br)C[C@]2(CNC(=O)O2)C1] | 377 |
| 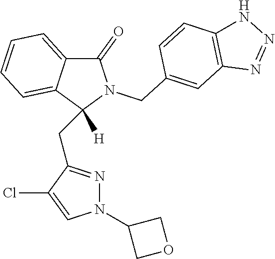 [SMILES: O=C1c2ccccc2[C@@H](Cc2nn(C3COC3)cc2Cl)N1Cc1ccc2[nH]nnc2c1] | 378 |
| 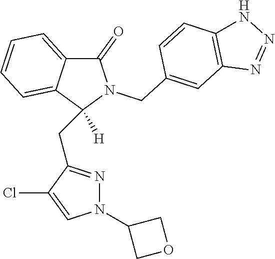 [SMILES: O=C1c2ccccc2[C@H](Cc2nn(C3COC3)cc2Cl)N1Cc1ccc2[nH]nnc2c1] | 379 |
| 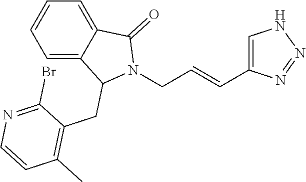 [SMILES: Cc1ccnc(Br)c1CC1c2ccccc2C(=O)N1C/C=C/c1c[nH]nn1] | 380 |
| 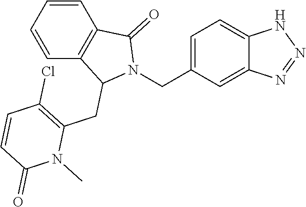 [SMILES: Cn1c(CC2c3ccccc3C(=O)N2Cc2ccc3[nH]nnc3c2)c(Cl)ccc1=O] | 381 |
| 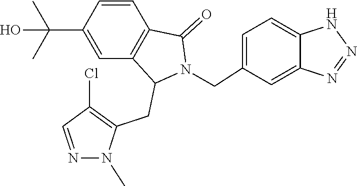 [SMILES: Cn1ncc(Cl)c1CC1c2cc(C(C)(C)O)ccc2C(=O)N1Cc1ccc2[nH]nnc2c1] | 382 |
| 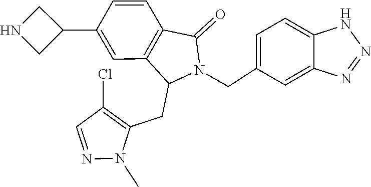 [SMILES: Cn1ncc(Cl)c1CC1c2cc(C3CNC3)ccc2C(=O)N1Cc1ccc2[nH]nnc2c1] | 383 |
| 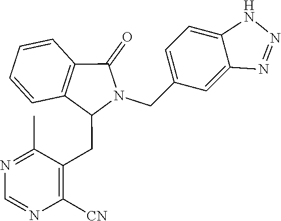 [SMILES: Cc1ncnc(C#N)c1CC1c2ccccc2C(=O)N1Cc1ccc2[nH]nnc2c1] | 384 |
| 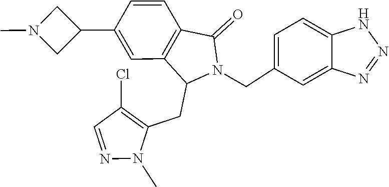 [SMILES: CN1CC(c2ccc3c(c2)C(Cc2c(Cl)cnn2C)N(Cc2ccc4[nH]nnc4c2)C3=O)C1] | 385 |
| 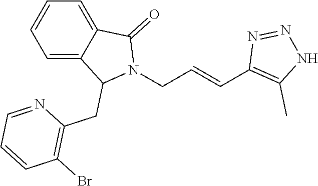 [SMILES: Cc1[nH]nnc1/C=C/CN1C(=O)c2ccccc2C1Cc1ncccc1Br] | 386 |
| 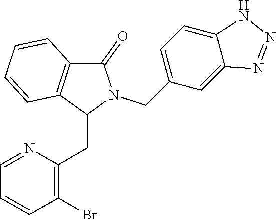 [SMILES: O=C1c2ccccc2C(Cc2ncccc2Br)N1Cc1ccc2[nH]nnc2c1] | 387 |
| 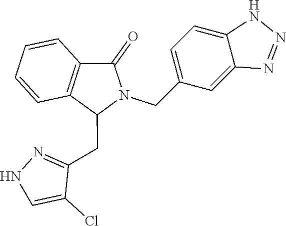 [SMILES: O=C1c2ccccc2C(Cc2n[nH]cc2Cl)N1Cc1ccc2[nH]nnc2c1] | 388 |
| 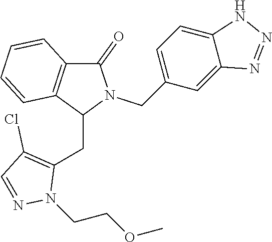 [SMILES: COCCn1ncc(Cl)c1CC1c2ccccc2C(=O)N1Cc1ccc2[nH]nnc2c1] | 389 |
| 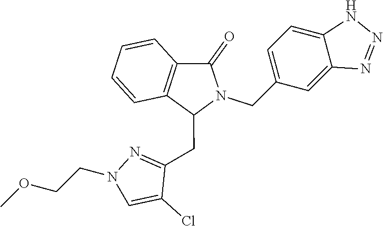 [SMILES: COCCn1cc(Cl)c(CC2c3ccccc3C(=O)N2Cc2ccc3[nH]nnc3c2)n1] | 390 |
| 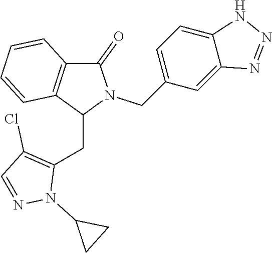 [SMILES: O=C1c2ccccc2C(Cc2c(Cl)cnn2C2CC2)N1Cc1ccc2[nH]nnc2c1] | 391 |
| 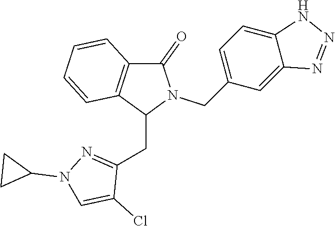 [SMILES: O=C1c2ccccc2C(Cc2nn(C3CC3)cc2Cl)N1Cc1ccc2[nH]nnc2c1] | 392 |
| 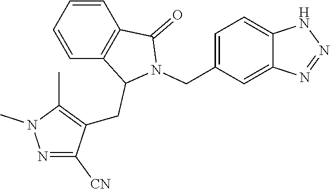 [SMILES: Cc1c(CC2c3ccccc3C(=O)N2Cc2ccc3[nH]nnc3c2)c(C#N)nn1C] | 393 |
| 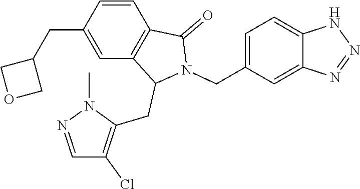 [SMILES: Cn1ncc(Cl)c1CC1c2cc(CC3COC3)ccc2C(=O)N1Cc1ccc2[nH]nnc2c1] | 394 |
| 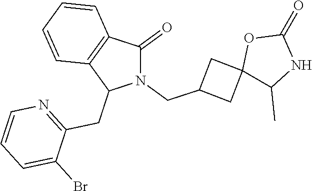 [SMILES: CC1NC(=O)OC12CC(CN1C(=O)c3ccccc3C1Cc1ncccc1Br)C2] | 395 |
| 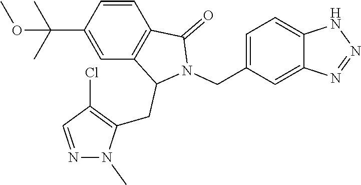 [SMILES: COC(C)(C)c1ccc2c(c1)C(Cc1c(Cl)cnn1C)N(Cc1ccc3[nH]nnc3c1)C2=O] | 396 |
| 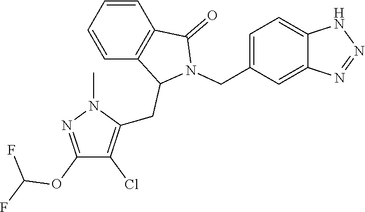 [SMILES: Cn1nc(OC(F)F)c(Cl)c1CC1c2ccccc2C(=O)N1Cc1ccc2[nH]nnc2c1] | 397 |
| 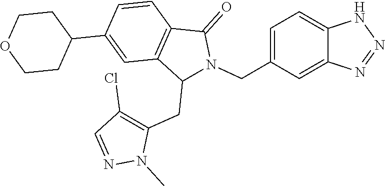 [SMILES: Cn1ncc(Cl)c1CC1c2cc(C3CCOCC3)ccc2C(=O)N1Cc1ccc2[nH]nnc2c1] | 398 |
| 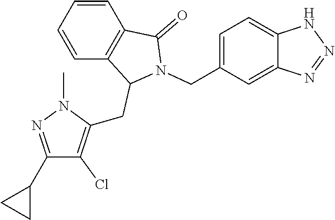 [SMILES: Cn1nc(C2CC2)c(Cl)c1CC1c2ccccc2C(=O)N1Cc1ccc2[nH]nnc2c1] | 399 |
| 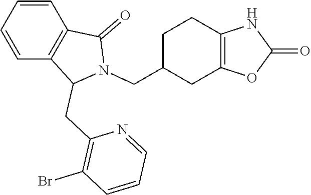 [SMILES: O=C1c2ccccc2C(Cc2ncccc2Br)N1CC1CCc2[nH]c(=O)oc2C1] | 400 |
| 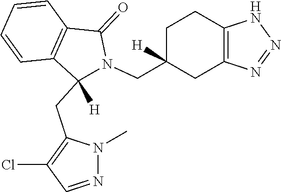 [SMILES: Cn1ncc(Cl)c1C[C@@H]1c2ccccc2C(=O)N1C[C@@H]1CCc2[nH]nnc2C1] | 401-1 |
| 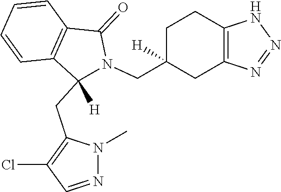 [SMILES: Cn1ncc(Cl)c1C[C@@H]1c2ccccc2C(=O)N1C[C@H]1CCc2[nH]nnc2C1] | 401-2 |
| 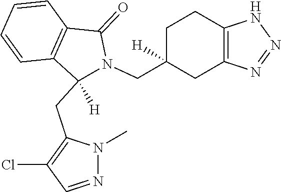 [SMILES: Cn1ncc(Cl)c1C[C@H]1c2ccccc2C(=O)N1C[C@H]1CCc2[nH]nnc2C1] | 402-1 |
| 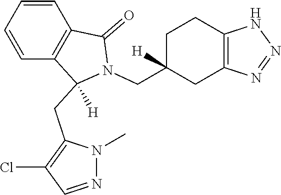 [SMILES: Cn1ncc(Cl)c1C[C@H]1c2ccccc2C(=O)N1C[C@@H]1CCc2[nH]nnc2C1] | 402-2 |
| 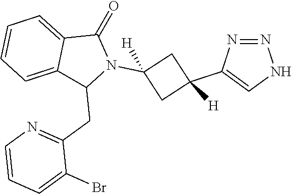 [SMILES: O=C1c2ccccc2C(Cc2ncccc2Br)N1[C@H]1C[C@H](c2c[nH]nn2)C1] | 403 |
| 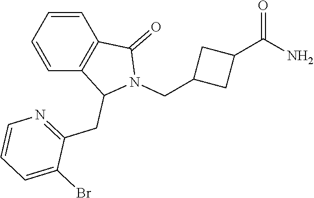 [SMILES: NC(=O)C1CC(CN2C(=O)c3ccccc3C2Cc2ncccc2Br)C1] | 404 |
| 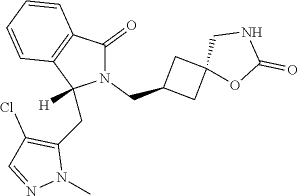 [SMILES: Cn1ncc(Cl)c1C[C@@H]1c2ccccc2C(=O)N1C[C@H]1C[C@]2(CNC(=O)O2)C1] | 405 |
| 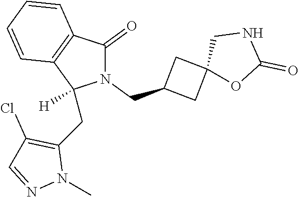 [SMILES: Cn1ncc(Cl)c1C[C@H]1c2ccccc2C(=O)N1C[C@H]1C[C@]2(CNC(=O)O2)C1] | 406 |
| 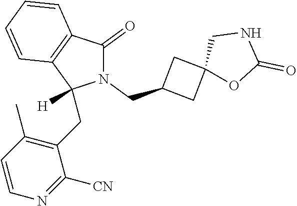 [SMILES: Cc1ccnc(C#N)c1C[C@@H]1c2ccccc2C(=O)N1C[C@H]1C[C@]2(CNC(=O)O2)C1] | 407 |
| 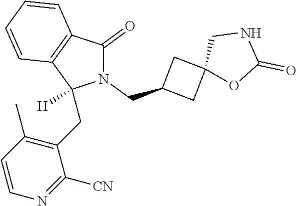 [SMILES: Cc1ccnc(C#N)c1C[C@H]1c2ccccc2C(=O)N1C[C@H]1C[C@]2(CNC(=O)O2)C1] | 408 |
| 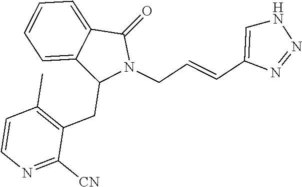 [SMILES: Cc1ccnc(C#N)c1CC1c2ccccc2C(=O)N1C/C=C/c1c[nH]nn1] | 409 |
| 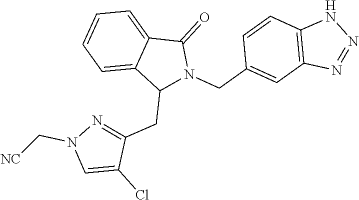 [SMILES: N#CCn1cc(Cl)c(CC2c3ccccc3C(=O)N2Cc2ccc3[nH]nnc3c2)n1] | 410 |
| 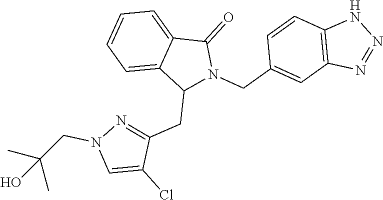 [SMILES: CC(C)(O)Cn1cc(Cl)c(CC2c3ccccc3C(=O)N2Cc2ccc3[nH]nnc3c2)n1] | 411 |
| 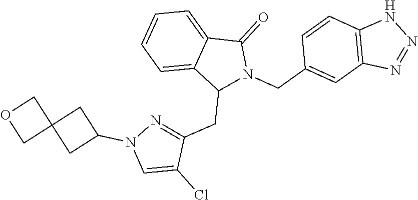 [SMILES: O=C1c2ccccc2C(Cc2nn(C3CC4(COC4)C3)cc2Cl)N1Cc1ccc2[nH]nnc2c1] | 412 |
| 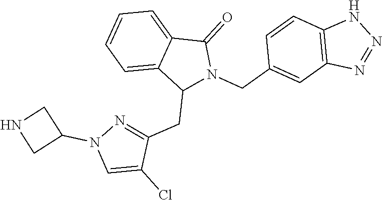 [SMILES: O=C1c2ccccc2C(Cc2nn(C3CNC3)cc2Cl)N1Cc1ccc2[nH]nnc2c1] | 413 |
| 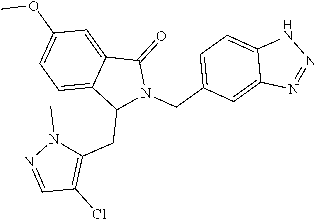 [SMILES: COc1ccc2c(c1)C(=O)N(Cc1ccc3[nH]nnc3c1)C2Cc1c(Cl)cnn1C] | 414 |
| 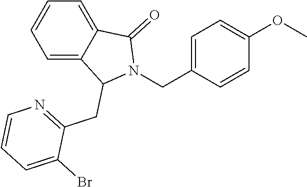 [SMILES: COc1ccc(CN2C(=O)c3ccccc3C2Cc2ncccc2Br)cc1] | 415 |
| 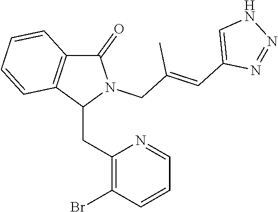 [SMILES: C/C(=C\c1c[nH]nn1)CN1C(=O)c2ccccc2C1Cc1ncccc1Br] | 416 |
| 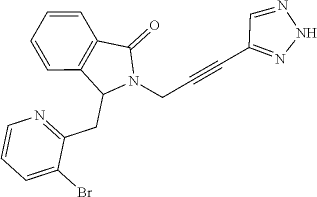 [SMILES: O=C1c2ccccc2C(Cc2ncccc2Br)N1CC#Cc1cn[nH]n1] | 417 |
| 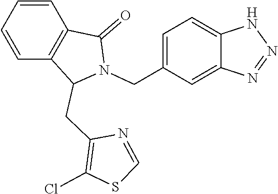 [SMILES: O=C1c2ccccc2C(Cc2ncsc2Cl)N1Cc1ccc2[nH]nnc2c1] | 418 |
| 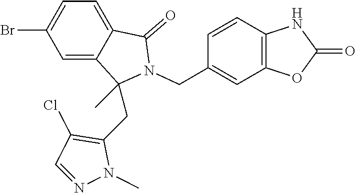 [SMILES: Cn1ncc(Cl)c1CC1(C)c2cc(Br)ccc2C(=O)N1Cc1ccc2[nH]c(=O)oc2c1] | 419 |
| 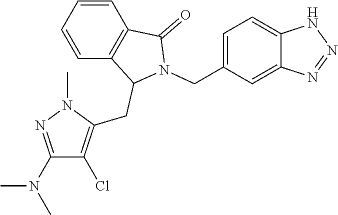 [SMILES: CN(C)c1nn(C)c(CC2c3ccccc3C(=O)N2Cc2ccc3[nH]nnc3c2)c1Cl] | 420 |
| 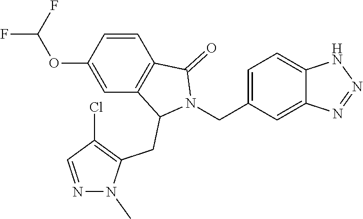 [SMILES: Cn1ncc(Cl)c1CC1c2cc(OC(F)F)ccc2C(=O)N1Cc1ccc2[nH]nnc2c1] | 421 |
| 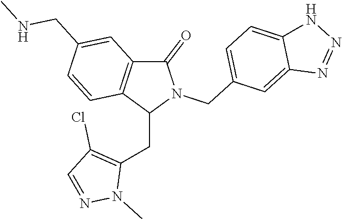 [SMILES: CNCc1ccc2c(c1)C(=O)N(Cc1ccc3[nH]nnc3c1)C2Cc1c(Cl)cnn1C] | 422 |
| 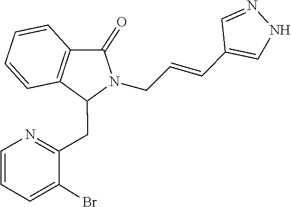 [SMILES: O=C1c2ccccc2C(Cc2ncccc2Br)N1C/C=C/c1cn[nH]c1] | 423 |
| 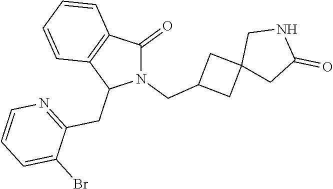 [SMILES: O=C1CC2(CN1)CC(CN1C(=O)c3ccccc3C1Cc1ncccc1Br)C2] | 424 |
| 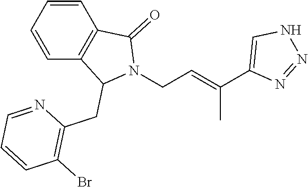 [SMILES: C/C(=C\CN1C(=O)c2ccccc2C1Cc1ncccc1Br)c1c[nH]nn1] | 425 |
| 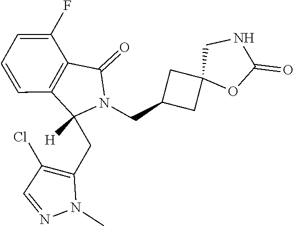 [SMILES: Cn1ncc(Cl)c1C[C@@H]1c2cccc(F)c2C(=O)N1C[C@H]1C[C@]2(CNC(=O)O2)C1] | 426 |
| 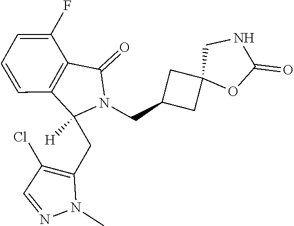 [SMILES: Cn1ncc(Cl)c1C[C@H]1c2cccc(F)c2C(=O)N1C[C@H]1C[C@]2(CNC(=O)O2)C1] | 427 |
| 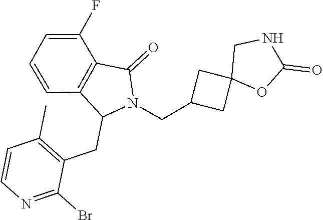 [SMILES: Cc1ccnc(Br)c1CC1c2cccc(F)c2C(=O)N1CC1CC2(CNC(=O)O2)C1] | 428 |
| 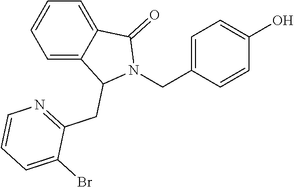 [SMILES: O=C1c2ccccc2C(Cc2ncccc2Br)N1Cc1ccc(O)cc1] | 429 |
| 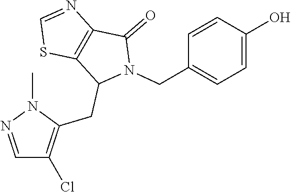 [SMILES: Cn1ncc(Cl)c1CC1c2scnc2C(=O)N1Cc1ccc(O)cc1] | 430 |
| 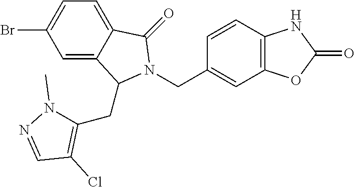 [SMILES: Cn1ncc(Cl)c1CC1c2cc(Br)ccc2C(=O)N1Cc1ccc2[nH]c(=O)oc2c1] | 431 |
| 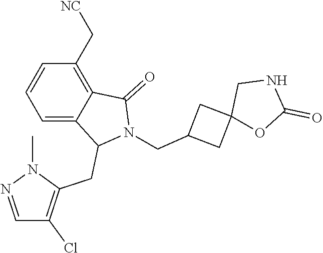 [SMILES: Cn1ncc(Cl)c1CC1c2cccc(CC#N)c2C(=O)N1CC1CC2(CNC(=O)O2)C1] | 432 |
| 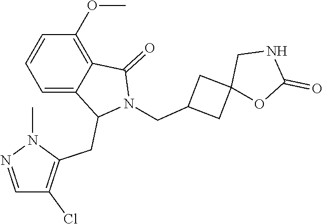 [SMILES: COc1cccc2c1C(=O)N(CC1CC3(CNC(=O)O3)C1)C2Cc1c(Cl)cnn1C] | 433 |
| 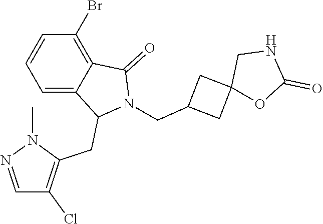 [SMILES: Cn1ncc(Cl)c1CC1c2cccc(Br)c2C(=O)N1CC1CC2(CNC(=O)O2)C1] | 434 |
| 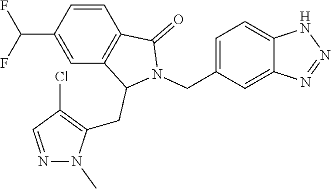 [SMILES: Cn1ncc(Cl)c1CC1c2cc(C(F)F)ccc2C(=O)N1Cc1ccc2[nH]nnc2c1] | 435 |
| 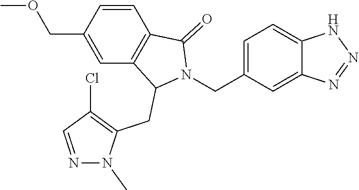 [SMILES: COCc1ccc2c(c1)C(Cc1c(Cl)cnn1C)N(Cc1ccc3[nH]nnc3c1)C2=O] | 436 |
| 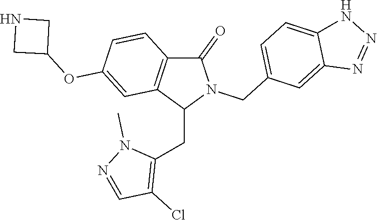 [SMILES: Cn1ncc(Cl)c1CC1c2cc(OC3CNC3)ccc2C(=O)N1Cc1ccc2[nH]nnc2c1] | 437 |
| 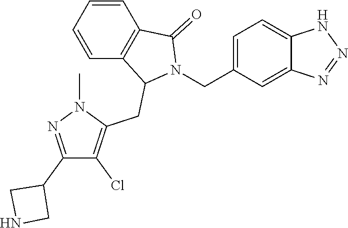 [SMILES: Cn1nc(C2CNC2)c(Cl)c1CC1c2ccccc2C(=O)N1Cc1ccc2[nH]nnc2c1] | 438 |
| 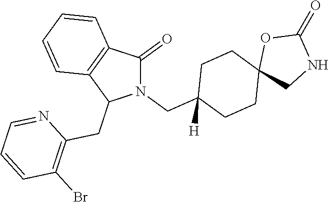 [SMILES: O=C1NC[C@]2(CC[C@H](CN3C(=O)c4ccccc4C3Cc3ncccc3Br)CC2)O1] | 439 |
| 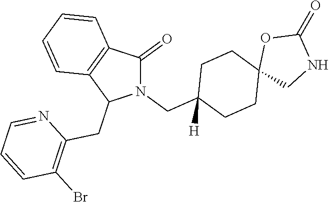 [SMILES: O=C1NC[C@]2(CC[C@@H](CN3C(=O)c4ccccc4C3Cc3ncccc3Br)CC2)O1] | 440 |
| 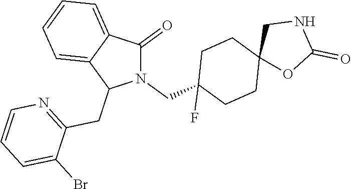 [SMILES: O=C1NC[C@]2(CC[C@](F)(CN3C(=O)c4ccccc4C3Cc3ncccc3Br)CC2)O1] | 441 |
| 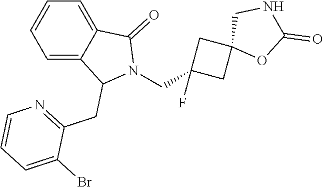 [SMILES: O=C1NC[C@]2(C[C@@](F)(CN3C(=O)c4ccccc4C3Cc3ncccc3Br)C2)O1] | 442 |
| 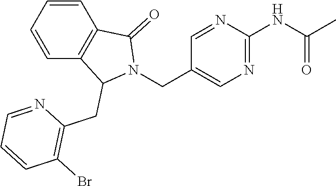 [SMILES: CC(=O)Nc1ncc(CN2C(=O)c3ccccc3C2Cc2ncccc2Br)cn1] | 443 |
| 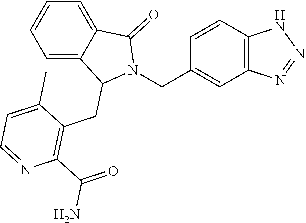 [SMILES: Cc1ccnc(C(N)=O)c1CC1c2ccccc2C(=O)N1Cc1ccc2[nH]nnc2c1] | 444 |
| 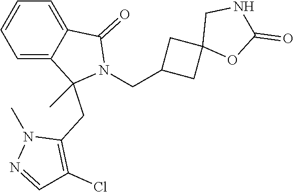 [SMILES: Cn1ncc(Cl)c1CC1(C)c2ccccc2C(=O)N1CC1CC2(CNC(=O)O2)C1] | 445 |
| 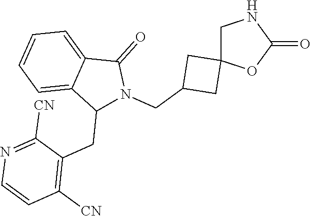 [SMILES: N#Cc1ccnc(C#N)c1CC1c2ccccc2C(=O)N1CC1CC2(CNC(=O)O2)C1] | 446 |
| 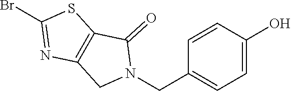 [SMILES: O=C1c2sc(Br)nc2CN1Cc1ccc(O)cc1] | 447 |
| 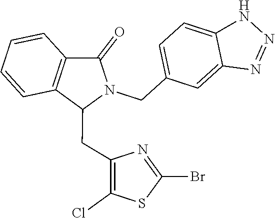 [SMILES: O=C1c2ccccc2C(Cc2nc(Br)sc2Cl)N1Cc1ccc2[nH]nnc2c1] | 448 |
| 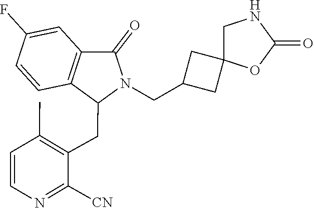 [SMILES: Cc1ccnc(C#N)c1CC1c2ccc(F)cc2C(=O)N1CC1CC2(CNC(=O)O2)C1] | 449 |
| 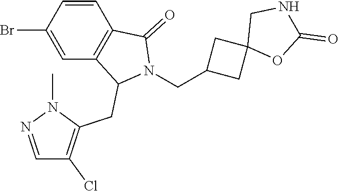 [SMILES: Cn1ncc(Cl)c1CC1c2cc(Br)ccc2C(=O)N1CC1CC2(CNC(=O)O2)C1] | 450 |
| 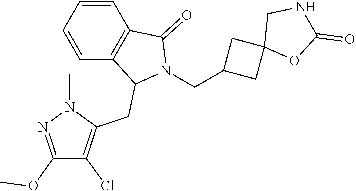 [SMILES: COc1nn(C)c(CC2c3ccccc3C(=O)N2CC2CC3(CNC(=O)O3)C2)c1Cl] | 451 |
| 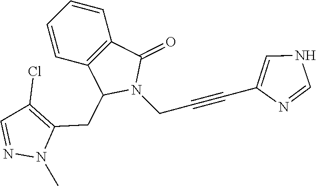 [SMILES: Cn1ncc(Cl)c1CC1c2ccccc2C(=O)N1CC#Cc1c[nH]cn1] | 452 |
| 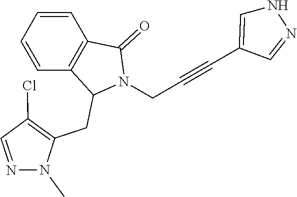 [SMILES: Cn1ncc(Cl)c1CC1c2ccccc2C(=O)N1CC#Cc1cn[nH]c1] | 453 |
| 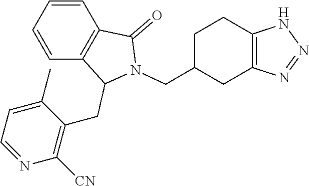 [SMILES: Cc1ccnc(C#N)c1CC1c2ccccc2C(=O)N1CC1CCc2[nH]nnc2C1] | 454 |
| 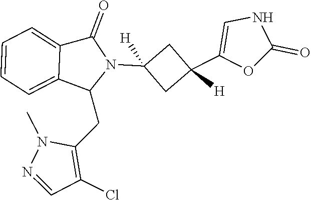 [SMILES: Cn1ncc(Cl)c1CC1c2ccccc2C(=O)N1[C@H]1C[C@H](c2c[nH]c(=O)o2)C1] | 455 |
| 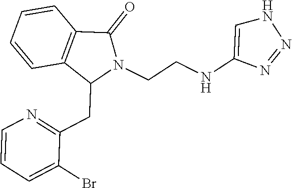 [SMILES: O=C1c2ccccc2C(Cc2ncccc2Br)N1CCNc1c[nH]nn1] | 456 |
| 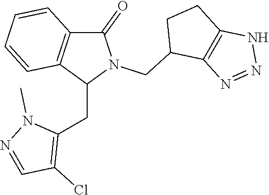 [SMILES: Cn1ncc(Cl)c1CC1c2ccccc2C(=O)N1CC1CCc2[nH]nnc21] | 457 |
| 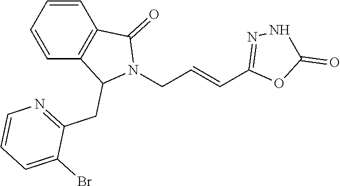 [SMILES: O=C1c2ccccc2C(Cc2ncccc2Br)N1C/C=C/c1n[nH]c(=O)o1] | 458 |
| 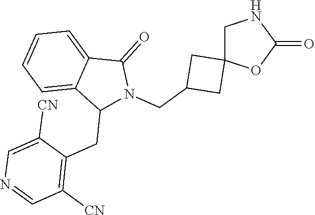 [SMILES: N#Cc1cncc(C#N)c1CC1c2ccccc2C(=O)N1CC1CC2(CNC(=O)O2)C1] | 459 |
| 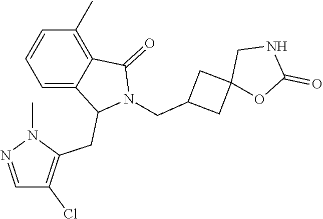 [SMILES: Cc1cccc2c1C(=O)N(CC1CC3(CNC(=O)O3)C1)C2Cc1c(Cl)cnn1C] | 460 |
| 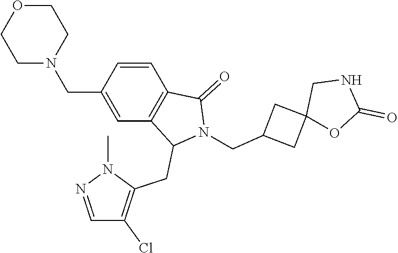 [SMILES: Cn1ncc(Cl)c1CC1c2cc(CN3CCOCC3)ccc2C(=O)N1CC1CC2(CNC(=O)O2)C1] | 461 |
| 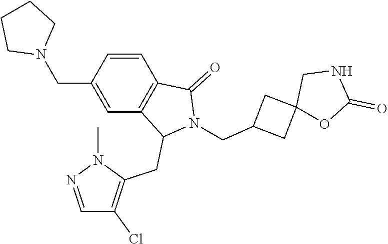 [SMILES: Cn1ncc(Cl)c1CC1c2cc(CN3CCCC3)ccc2C(=O)N1CC1CC2(CNC(=O)O2)C1] | 462 |
| 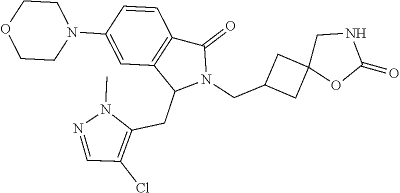 [SMILES: Cn1ncc(Cl)c1CC1c2cc(N3CCOCC3)ccc2C(=O)N1CC1CC2(CNC(=O)O2)C1] | 463 |
| 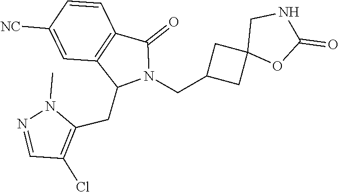 [SMILES: Cn1ncc(Cl)c1CC1c2cc(C#N)ccc2C(=O)N1CC1CC2(CNC(=O)O2)C1] | 464 |
| 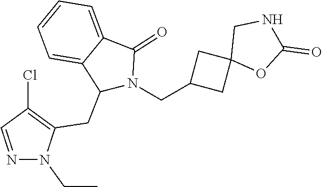 [SMILES: CCn1ncc(Cl)c1CC1c2ccccc2C(=O)N1CC1CC2(CNC(=O)O2)C1] | 465 |
| 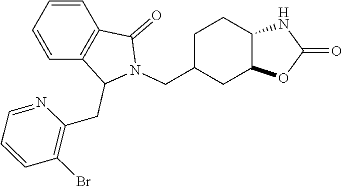 [SMILES: O=C1N[C@H]2CCC(CN3C(=O)c4ccccc4C3Cc3ncccc3Br)C[C@@H]2O1] | 466 |
| 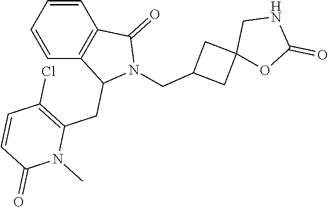 [SMILES: Cn1c(CC2c3ccccc3C(=O)N2CC2CC3(CNC(=O)O3)C2)c(Cl)ccc1=O] | 467 |
| 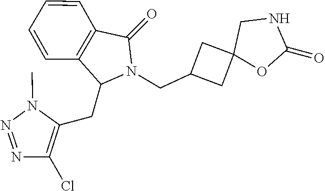 [SMILES: Cn1nnc(Cl)c1CC1c2ccccc2C(=O)N1CC1CC2(CNC(=O)O2)C1] | 468 |
| 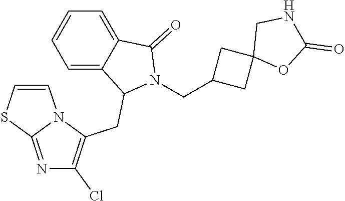 [SMILES: O=C1NCC2(CC(CN3C(=O)c4ccccc4C3Cc3c(Cl)nc4sccn34)C2)O1] | 469 |
| 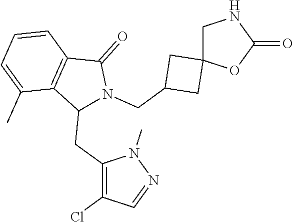 [SMILES: Cc1cccc2c1C(Cc1c(Cl)cnn1C)N(CC1CC3(CNC(=O)O3)C1)C2=O] | 470 |
| 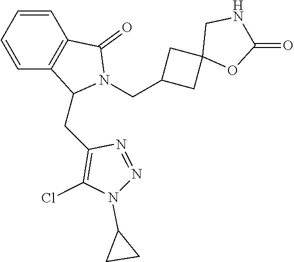 [SMILES: O=C1NCC2(CC(CN3C(=O)c4ccccc4C3Cc3nnn(C4CC4)c3Cl)C2)O1] | 471 |
| 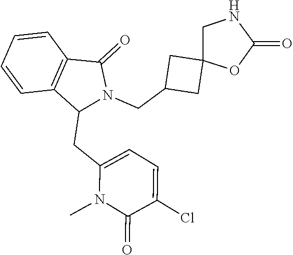 [SMILES: Cn1c(CC2c3ccccc3C(=O)N2CC2CC3(CNC(=O)O3)C2)ccc(Cl)c1=O] | 472 |
| 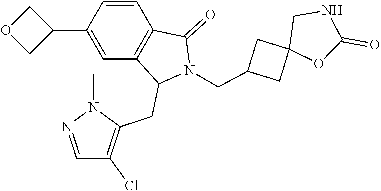 [SMILES: Cn1ncc(Cl)c1CC1c2cc(C3COC3)ccc2C(=O)N1CC1CC2(CNC(=O)O2)C1] | 473 |
| 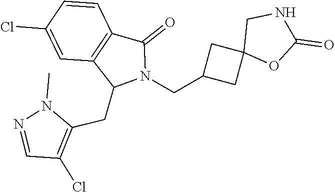 [SMILES: Cn1ncc(Cl)c1CC1c2cc(Cl)ccc2C(=O)N1CC1CC2(CNC(=O)O2)C1] | 474 |
| 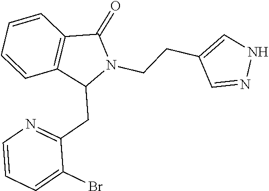 [SMILES: O=C1c2ccccc2C(Cc2ncccc2Br)N1CCc1cn[nH]c1] | 475 |
| 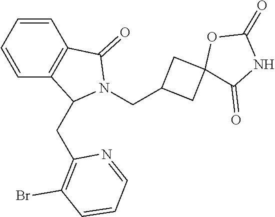 [SMILES: O=C1NC(=O)C2(CC(CN3C(=O)c4ccccc4C3Cc3ncccc3Br)C2)O1] | 476 |
| 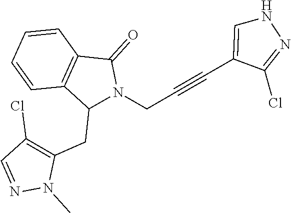 [SMILES: Cn1ncc(Cl)c1CC1c2ccccc2C(=O)N1CC#Cc1c[nH]nc1Cl] | 477 |
| 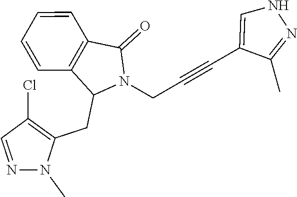 [SMILES: Cc1n[nH]cc1C#CCN1C(=O)c2ccccc2C1Cc1c(Cl)cnn1C] | 478 |
| 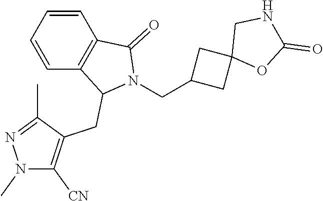 [SMILES: Cc1nn(C)c(C#N)c1CC1c2ccccc2C(=O)N1CC1CC2(CNC(=O)O2)C1] | 479 |
| 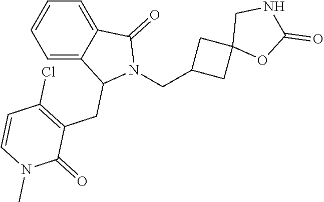 [SMILES: Cn1ccc(Cl)c(CC2c3ccccc3C(=O)N2CC2CC3(CNC(=O)O3)C2)c1=O] | 480 |
| 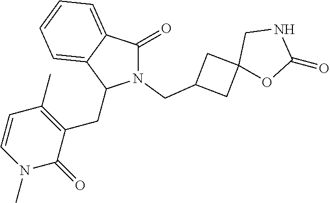 [SMILES: Cc1ccn(C)c(=O)c1CC1c2ccccc2C(=O)N1CC1CC2(CNC(=O)O2)C1] | 481 |
| 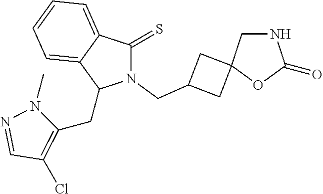 [SMILES: Cn1ncc(Cl)c1CC1c2ccccc2C(=S)N1CC1CC2(CNC(=O)O2)C1] | 482 |
| 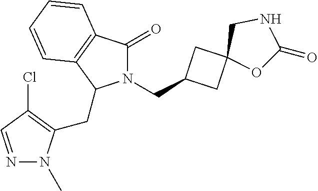 [SMILES: Cn1ncc(Cl)c1CC1c2ccccc2C(=O)N1C[C@H]1C[C@@]2(CNC(=O)O2)C1] | 483 |
| 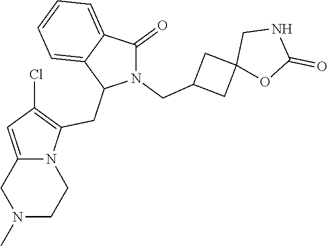 [SMILES: CN1CCn2c(cc(Cl)c2CC2c3ccccc3C(=O)N2CC2CC3(CNC(=O)O3)C2)C1] | 484 |
| 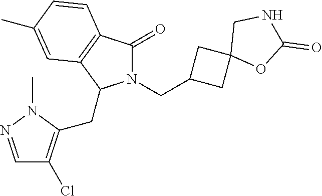 [SMILES: Cc1ccc2c(c1)C(Cc1c(Cl)cnn1C)N(CC1CC3(CNC(=O)O3)C1)C2=O] | 485 |
| 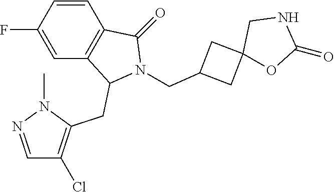 [SMILES: Cn1ncc(Cl)c1CC1c2cc(F)ccc2C(=O)N1CC1CC2(CNC(=O)O2)C1] | 486 |
| 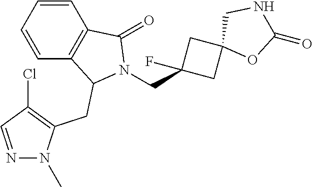 [SMILES: Cn1ncc(Cl)c1CC1c2ccccc2C(=O)N1C[C@]1(F)C[C@@]2(CNC(=O)O2)C1] | 487 |
| 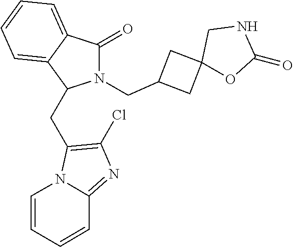 [SMILES: O=C1NCC2(CC(CN3C(=O)c4ccccc4C3Cc3c(Cl)nc4ccccn34)C2)O1] | 488 |
| 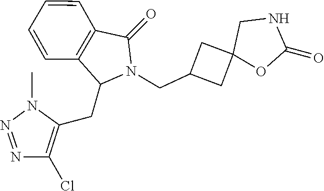 [SMILES: Cn1nnc(Cl)c1CC1c2ccccc2C(=O)N1CC1CC2(CNC(=O)O2)C1] | 489 |
| 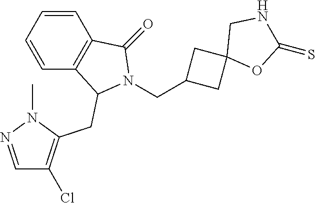 [SMILES: Cn1ncc(Cl)c1CC1c2ccccc2C(=O)N1CC1CC2(CNC(=S)O2)C1] | 490 |
| 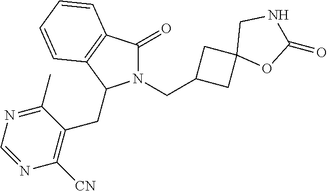 [SMILES: Cc1ncnc(C#N)c1CC1c2ccccc2C(=O)N1CC1CC2(CNC(=O)O2)C1] | 491 |
| 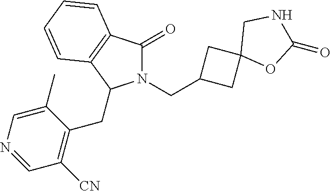 [SMILES: Cc1cncc(C#N)c1CC1c2ccccc2C(=O)N1CC1CC2(CNC(=O)O2)C1] | 492 |
| 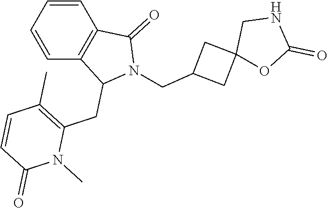 [SMILES: Cc1ccc(=O)n(C)c1CC1c2ccccc2C(=O)N1CC1CC2(CNC(=O)O2)C1] | 493 |
| 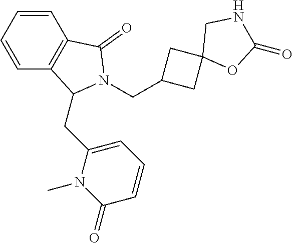 [SMILES: Cn1c(CC2c3ccccc3C(=O)N2CC2CC3(CNC(=O)O3)C2)cccc1=O] | 494 |
| 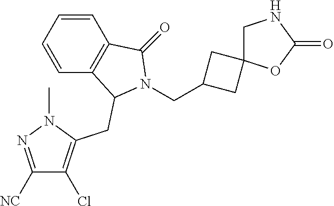 [SMILES: Cn1nc(C#N)c(Cl)c1CC1c2ccccc2C(=O)N1CC1CC2(CNC(=O)O2)C1] | 495 |
| 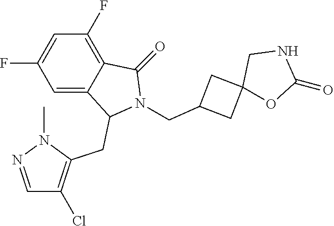 [SMILES: Cn1ncc(Cl)c1CC1c2cc(F)cc(F)c2C(=O)N1CC1CC2(CNC(=O)O2)C1] | 496 |
| 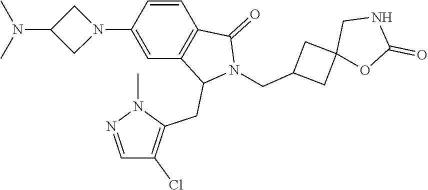 [SMILES: CN(C)C1CN(c2ccc3c(c2)C(Cc2c(Cl)cnn2C)N(CC2CC4(CNC(=O)O4)C2)C3=O)C1] | 497 |
| 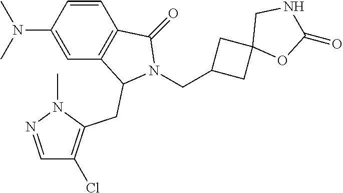 [SMILES: CN(C)c1ccc2c(c1)C(Cc1c(Cl)cnn1C)N(CC1CC3(CNC(=O)O3)C1)C2=O] | 498 |
| 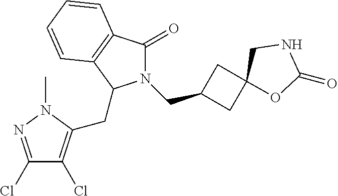 [SMILES: Cn1nc(Cl)c(Cl)c1CC1c2ccccc2C(=O)N1C[C@H]1C[C@@]2(CNC(=O)O2)C1] | 499 |
| 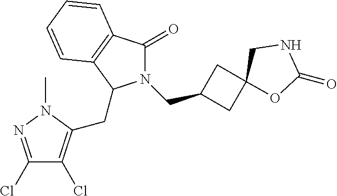 [SMILES: Cn1nc(Cl)c(Cl)c1CC1c2ccccc2C(=O)N1C[C@H]1C[C@@]2(CNC(=O)O2)C1] | 500 |
| 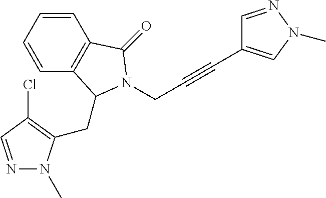 [SMILES: Cn1cc(C#CCN2C(=O)c3ccccc3C2Cc2c(Cl)cnn2C)cn1] | 501 |
| 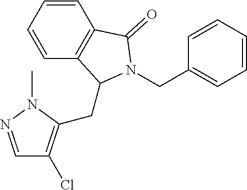 [SMILES: Cn1ncc(Cl)c1CC1c2ccccc2C(=O)N1Cc1ccccc1] | 502 |
| 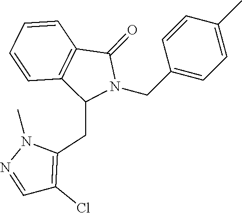 [SMILES: Cc1ccc(CN2C(=O)c3ccccc3C2Cc2c(Cl)cnn2C)cc1] | 503 |
| 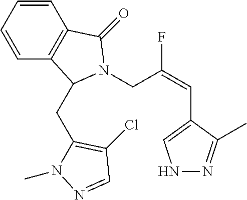 [SMILES: Cc1n[nH]cc1/C=C(/F)CN1C(=O)c2ccccc2C1Cc1c(Cl)cnn1C] | 504 |
| 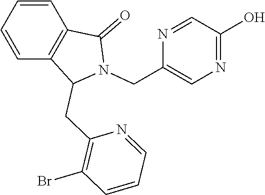 [SMILES: O=C1c2ccccc2C(Cc2ncccc2Br)N1Cc1cnc(O)cn1] | 505 |
| 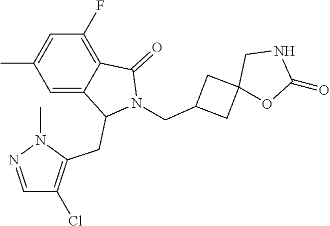 [SMILES: Cc1cc(F)c2c(c1)C(Cc1c(Cl)cnn1C)N(CC1CC3(CNC(=O)O3)C1)C2=O] | 506 |
| 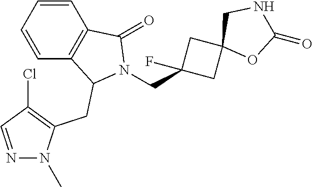 [SMILES: Cn1ncc(Cl)c1CC1c2ccccc2C(=O)N1C[C@]1(F)C[C@]2(CNC(=O)O2)C1] | 507 |
| 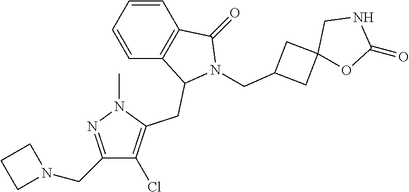 [SMILES: Cn1nc(CN2CCC2)c(Cl)c1CC1c2ccccc2C(=O)N1CC1CC2(CNC(=O)O2)C1] | 508 |
| 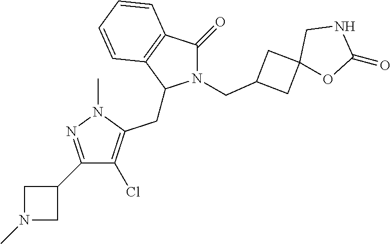 [SMILES: CN1CC(c2nn(C)c(CC3c4ccccc4C(=O)N3CC3CC4(CNC(=O)O4)C3)c2Cl)C1] | 509 |
| 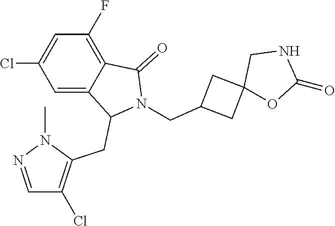 [SMILES: Cn1ncc(Cl)c1CC1c2cc(Cl)cc(F)c2C(=O)N1CC1CC2(CNC(=O)O2)C1] | 510 |
| 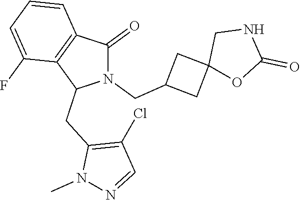 [SMILES: Cn1ncc(Cl)c1CC1c2c(F)cccc2C(=O)N1CC1CC2(CNC(=O)O2)C1] | 511 |
| 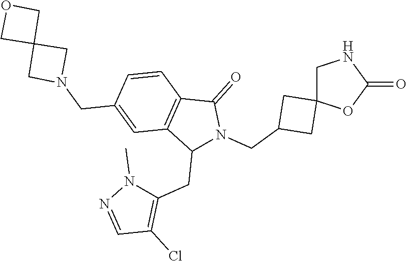 [SMILES: Cn1ncc(Cl)c1CC1c2cc(CN3CC4(COC4)C3)ccc2C(=O)N1CC1CC2(CNC(=O)O2)C1] | 512 |
| 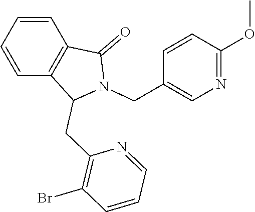 [SMILES: COc1ccc(CN2C(=O)c3ccccc3C2Cc2ncccc2Br)cn1] | 513 |
| 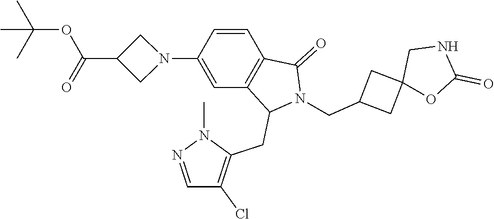 [SMILES: Cn1ncc(Cl)c1CC1c2cc(N3CC(C(=O)OC(C)(C)C)C3)ccc2C(=O)N1CC1CC2(CNC(=O)O2)C1] | 514 |
| 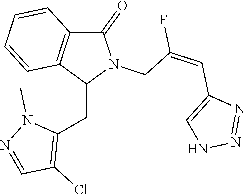 [SMILES: Cn1ncc(Cl)c1CC1c2ccccc2C(=O)N1C/C(F)=C\c1c[nH]nn1] | 515 |
| 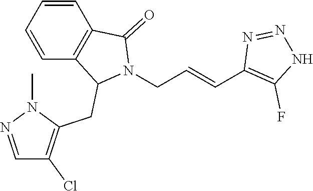 [SMILES: Cn1ncc(Cl)c1CC1c2ccccc2C(=O)N1C/C=C/c1nn[nH]c1F] | 516 |
| 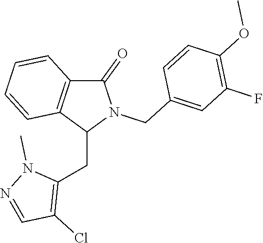 [SMILES: COc1ccc(CN2C(=O)c3ccccc3C2Cc2c(Cl)cnn2C)cc1F] | 517 |
| 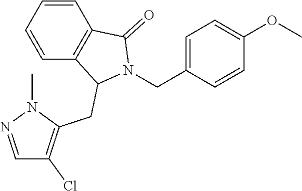 [SMILES: COc1ccc(CN2C(=O)c3ccccc3C2Cc2c(Cl)cnn2C)cc1] | 518 |
| 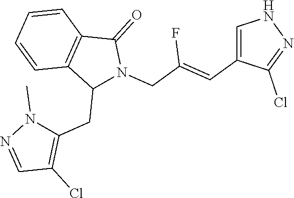 [SMILES: Cn1ncc(Cl)c1CC1c2ccccc2C(=O)N1C/C(F)=C/c1c[nH]nc1Cl] | 519 |
| 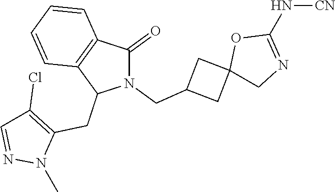 [SMILES: Cn1ncc(Cl)c1CC1c2ccccc2C(=O)N1CC1CC2(CN=C(NC#N)O2)C1] | 520 |
| 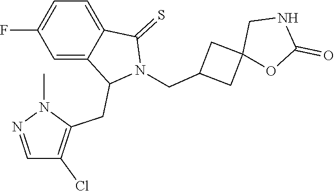 [SMILES: Cn1ncc(Cl)c1CC1c2cc(F)ccc2C(=S)N1CC1CC2(CNC(=O)O2)C1] | 521 |
| 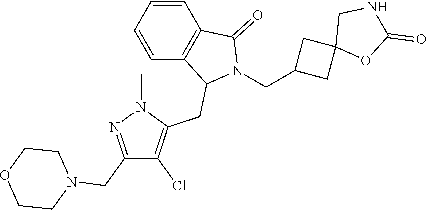 [SMILES: Cn1nc(CN2CCOCC2)c(Cl)c1CC1c2ccccc2C(=O)N1CC1CC2(CNC(=O)O2)C1] | 522 |
| 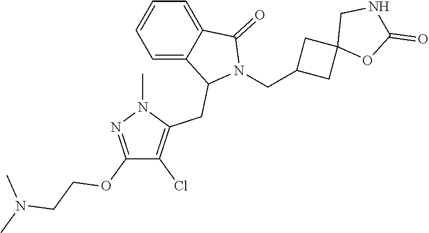 [SMILES: CN(C)CCOc1nn(C)c(CC2c3ccccc3C(=O)N2CC2CC3(CNC(=O)O3)C2)c1Cl] | 523 |
| 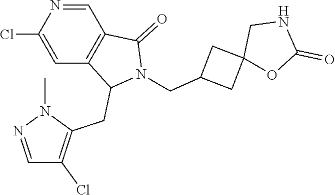 [SMILES: Cn1ncc(Cl)c1CC1c2cc(Cl)ncc2C(=O)N1CC1CC2(CNC(=O)O2)C1] | 524 |
| 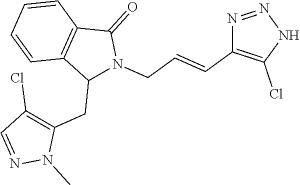 [SMILES: Cn1ncc(Cl)c1CC1c2ccccc2C(=O)N1C/C=C/c1nn[nH]c1Cl] | 525 |
| 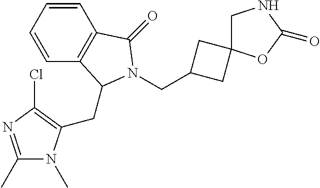 [SMILES: Cc1nc(Cl)c(CC2c3ccccc3C(=O)N2CC2CC3(CNC(=O)O3)C2)n1C] | 526 |
| 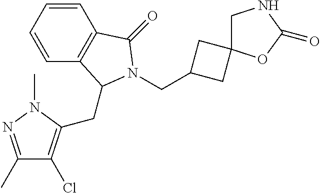 [SMILES: Cc1nn(C)c(CC2c3ccccc3C(=O)N2CC2CC3(CNC(=O)O3)C2)c1Cl] | 527 |
| 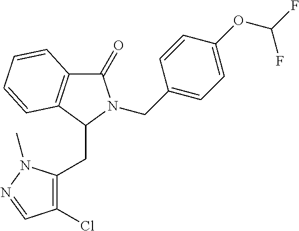 [SMILES: Cn1ncc(Cl)c1CC1c2ccccc2C(=O)N1Cc1ccc(OC(F)F)cc1] | 528 |
| 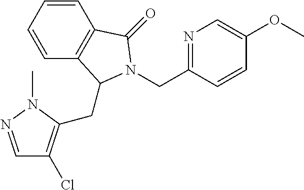 [SMILES: COc1ccc(CN2C(=O)c3ccccc3C2Cc2c(Cl)cnn2C)nc1] | 529 |
| 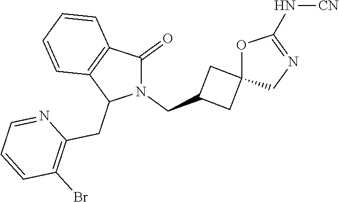 | 530 |
| 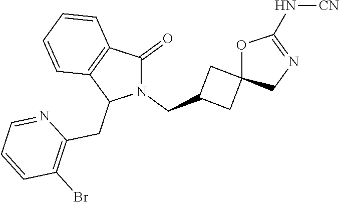 | 531 |
| 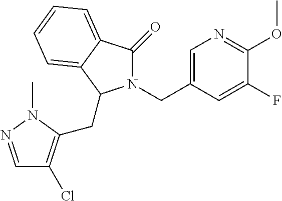 [SMILES: COc1ncc(CN2C(=O)c3ccccc3C2Cc2c(Cl)cnn2C)cc1F] | 532 |
| 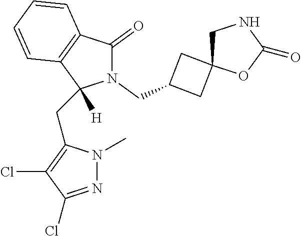 [SMILES: Cn1nc(Cl)c(Cl)c1C[C@@H]1c2ccccc2C(=O)N1C[C@H]1C[C@]2(CNC(=O)O2)C1] | 533 |
| 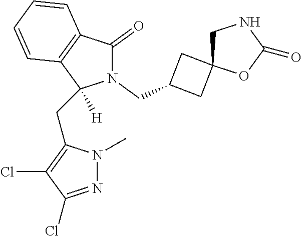 [SMILES: Cn1nc(Cl)c(Cl)c1C[C@H]1c2ccccc2C(=O)N1C[C@H]1C[C@]2(CNC(=O)O2)C1] | 534 |
| 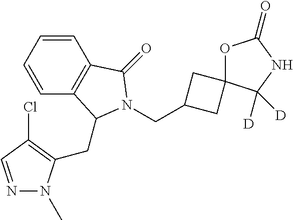 [SMILES: [2H]C1([2H])NC(=O)OC12CC(CN1C(=O)c3ccccc3C1Cc1c(Cl)cnn1C)C2] | 535 |
| 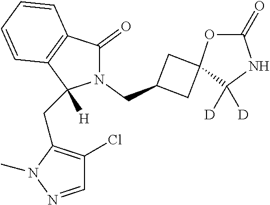 [SMILES: [2H]C1([2H])NC(=O)O[C@]12C[C@@H](CN1C(=O)c3ccccc3[C@H]1Cc1c(Cl)cnn1C)C2] | 536 |
| 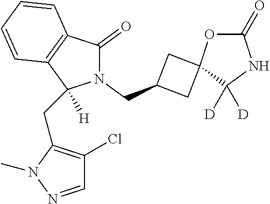 [SMILES: [2H]C1([2H])NC(=O)O[C@]12C[C@@H](CN1C(=O)c3ccccc3[C@@H]1Cc1c(Cl)cnn1C)C2] | 537 |
| 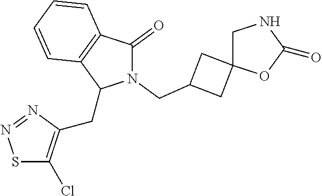 [SMILES: O=C1NCC2(CC(CN3C(=O)c4ccccc4C3Cc3nnsc3Cl)C2)O1] | 538 |
| 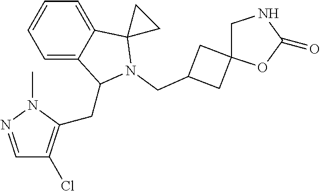 [SMILES: Cn1ncc(Cl)c1CC1c2ccccc2C2(CC2)N1CC1CC2(CNC(=O)O2)C1] | 539 |
| 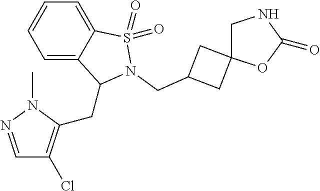 [SMILES: Cn1ncc(Cl)c1CC1c2ccccc2S(=O)(=O)N1CC1CC2(CNC(=O)O2)C1] | 540 |
| 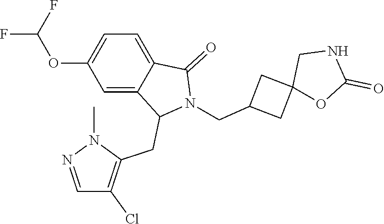 [SMILES: Cn1ncc(Cl)c1CC1c2cc(OC(F)F)ccc2C(=O)N1CC1CC2(CNC(=O)O2)C1] | 541 |
| 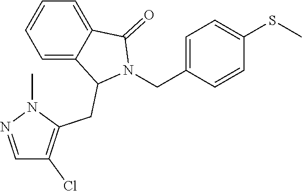 [SMILES: CSc1ccc(CN2C(=O)c3ccccc3C2Cc2c(Cl)cnn2C)cc1] | 542 |
| 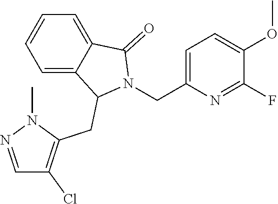 [SMILES: COc1ccc(CN2C(=O)c3ccccc3C2Cc2c(Cl)cnn2C)nc1F] | 543 |
| 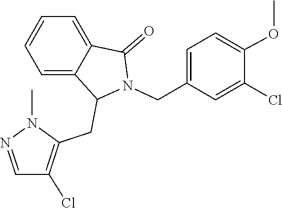 [SMILES: COc1ccc(CN2C(=O)c3ccccc3C2Cc2c(Cl)cnn2C)cc1Cl] | 544 |
| 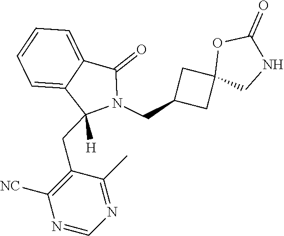 [SMILES: Cc1ncnc(C#N)c1C[C@@H]1c2ccccc2C(=O)N1C[C@H]1C[C@]2(CNC(=O)O2)C1] | 545 |
| 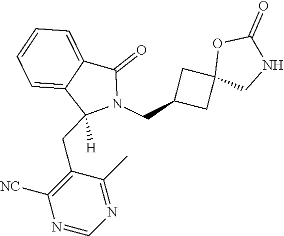 [SMILES: Cc1ncnc(C#N)c1C[C@H]1c2ccccc2C(=O)N1C[C@H]1C[C@]2(CNC(=O)O2)C1] | 546 |
| 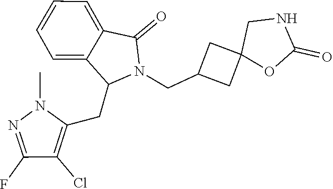 [SMILES: Cn1nc(F)c(Cl)c1CC1c2ccccc2C(=O)N1CC1CC2(CNC(=O)O2)C1] | 547 |
| 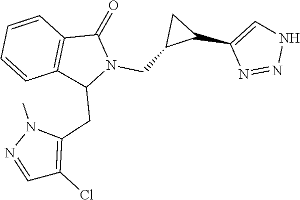 | 548 |
| 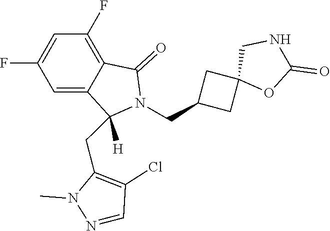 [SMILES: Cn1ncc(Cl)c1C[C@@H]1c2cc(F)cc(F)c2C(=O)N1C[C@H]1C[C@]2(CNC(=O)O2)C1] | 549 |
| 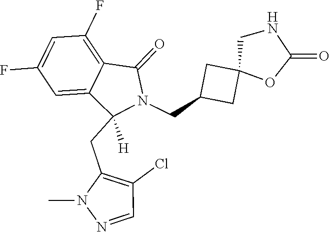 [SMILES: Cn1ncc(Cl)c1C[C@H]1c2cc(F)cc(F)c2C(=O)N1C[C@H]1C[C@]2(CNC(=O)O2)C1] | 550 |
| 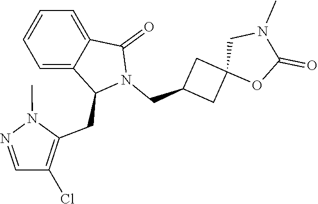 [SMILES: CN1C[C@]2(C[C@H](CN3C(=O)c4ccccc4[C@@H]3Cc3c(Cl)cnn3C)C2)OC1=O] | 551 |
| 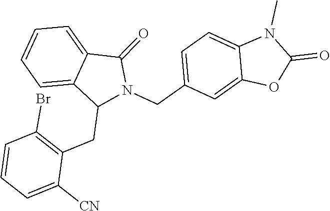 [SMILES: Cn1c(=O)oc2cc(CN3C(=O)c4ccccc4C3Cc3c(Br)cccc3C#N)ccc21] | 552 |
| 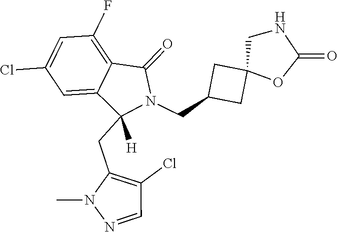 [SMILES: Cn1ncc(Cl)c1C[C@@H]1c2cc(Cl)cc(F)c2C(=O)N1C[C@H]1C[C@]2(CNC(=O)O2)C1] | 553 |
| 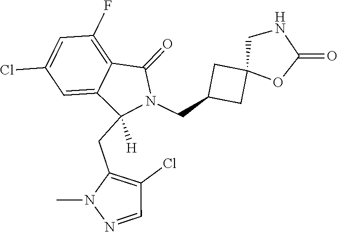 [SMILES: Cn1ncc(Cl)c1C[C@H]1c2cc(Cl)cc(F)c2C(=O)N1C[C@H]1C[C@]2(CNC(=O)O2)C1] | 554 |
| 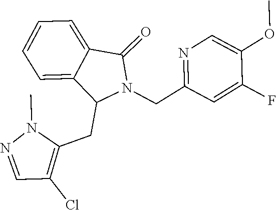 [SMILES: COc1cnc(CN2C(=O)c3ccccc3C2Cc2c(Cl)cnn2C)cc1F] | 555 |
| 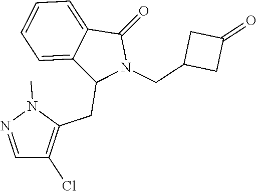 [SMILES: Cn1ncc(Cl)c1CC1c2ccccc2C(=O)N1CC1CC(=O)C1] | 556 |
| 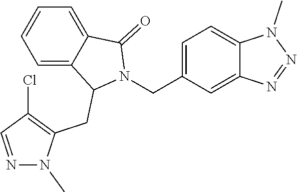 [SMILES: Cn1ncc(Cl)c1CC1c2ccccc2C(=O)N1Cc1ccc2c(c1)nnn2C] | 557 |
| 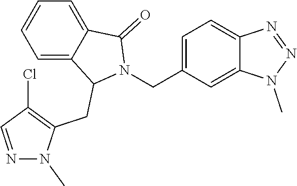 [SMILES: Cn1ncc(Cl)c1CC1c2ccccc2C(=O)N1Cc1ccc2nnn(C)c2c1] | 558 |
| 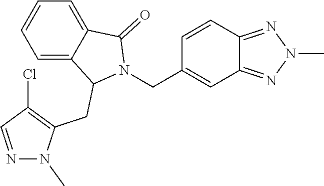 [SMILES: Cn1nc2ccc(CN3C(=O)c4ccccc4C3Cc3c(Cl)cnn3C)cc2n1] | 559 |
| 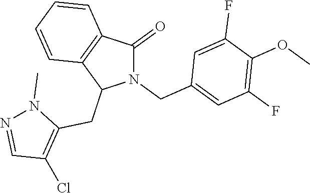 [SMILES: COc1c(F)cc(CN2C(=O)c3ccccc3C2Cc2c(Cl)cnn2C)cc1F] | 560 |
| 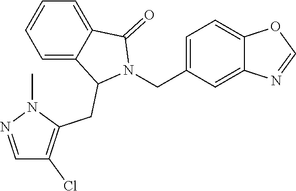 [SMILES: Cn1ncc(Cl)c1CC1c2ccccc2C(=O)N1Cc1ccc2ocnc2c1] | 561 |
| 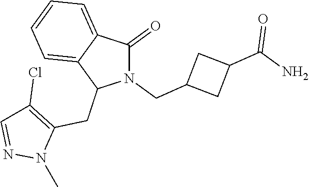 [SMILES: Cn1ncc(Cl)c1CC1c2ccccc2C(=O)N1CC1CC(C(N)=O)C1] | 562 |
| 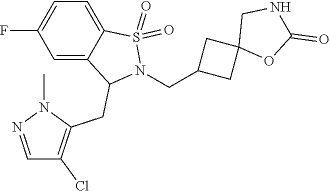 [SMILES: Cn1ncc(Cl)c1CC1c2cc(F)ccc2S(=O)(=O)N1CC1CC2(CNC(=O)O2)C1] | 563 |
| 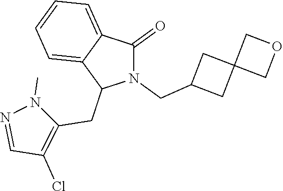 [SMILES: Cn1ncc(Cl)c1CC1c2ccccc2C(=O)N1CC1CC2(COC2)C1] | 564 |
| 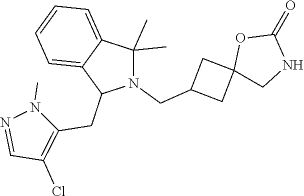 [SMILES: Cn1ncc(Cl)c1CC1c2ccccc2C(C)(C)N1CC1CC2(CNC(=O)O2)C1] | 565 |
| 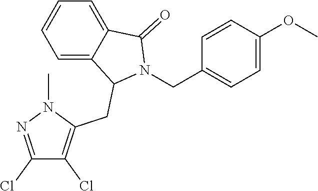 [SMILES: COc1ccc(CN2C(=O)c3ccccc3C2Cc2c(Cl)c(Cl)nn2C)cc1] | 566 |
| 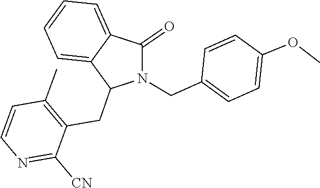 [SMILES: COc1ccc(CN2C(=O)c3ccccc3C2Cc2c(C)ccnc2C#N)cc1] | 567 |
| 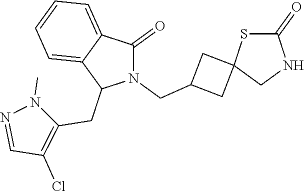 [SMILES: Cn1ncc(Cl)c1CC1c2ccccc2C(=O)N1CC1CC2(CNC(=O)S2)C1] | 568 |
| 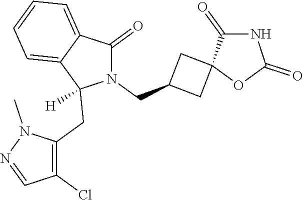 [SMILES: Cn1ncc(Cl)c1C[C@H]1c2ccccc2C(=O)N1C[C@H]1C[C@]2(C1)OC(=O)NC2=O] | 569 |
| 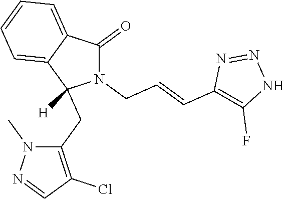 [SMILES: Cn1ncc(Cl)c1C[C@@H]1c2ccccc2C(=O)N1C/C=C/c1nn[nH]c1F] | 570 |
| 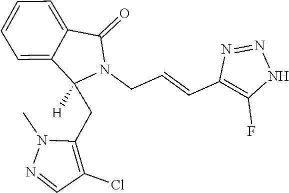 [SMILES: Cn1ncc(Cl)c1C[C@H]1c2ccccc2C(=O)N1C/C=C/c1nn[nH]c1F] | 571 |
| 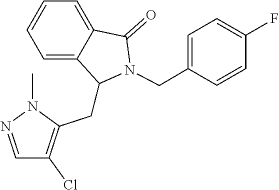 [SMILES: Cn1ncc(Cl)c1CC1c2ccccc2C(=O)N1Cc1ccc(F)cc1] | 572 |
| 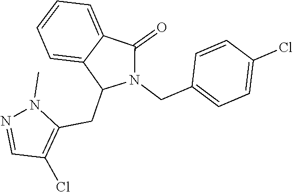 [SMILES: Cn1ncc(Cl)c1CC1c2ccccc2C(=O)N1Cc1ccc(Cl)cc1] | 573 |
| 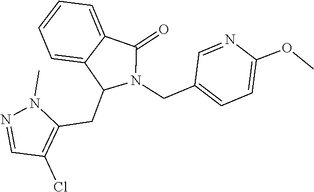 [SMILES: COc1ccc(CN2C(=O)c3ccccc3C2Cc2c(Cl)cnn2C)cn1] | 574 |
| 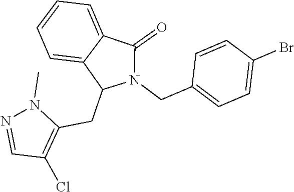 [SMILES: Cn1ncc(Cl)c1CC1c2ccccc2C(=O)N1Cc1ccc(Br)cc1] | 575 |
| 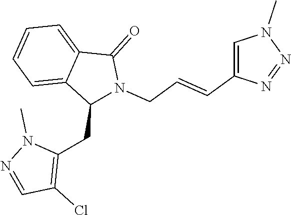 [SMILES: Cn1cc(/C=C/CN2C(=O)c3ccccc3[C@@H]2Cc2c(Cl)cnn2C)nn1] | 576-1 |
| 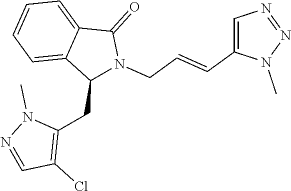 [SMILES: Cn1nncc1/C=C/CN1C(=O)c2ccccc2[C@@H]1Cc1c(Cl)cnn1C] | 576-2 |
| 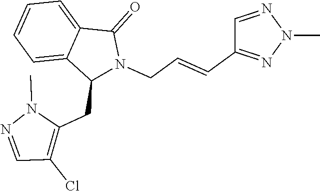 [SMILES: Cn1ncc(/C=C/CN2C(=O)c3ccccc3[C@@H]2Cc2c(Cl)cnn2C)n1] | 577 |
| 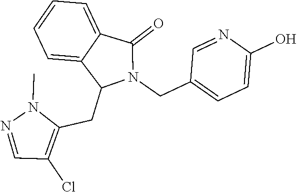 [SMILES: Cn1ncc(Cl)c1CC1c2ccccc2C(=O)N1Cc1ccc(O)nc1] | 578 |
| 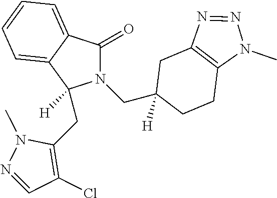 [SMILES: Cn1ncc(Cl)c1C[C@H]1c2ccccc2C(=O)N1C[C@@H]1CCc2c(nnn2C)C1] | 579 |
| 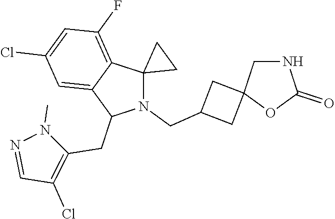 [SMILES: Cn1ncc(Cl)c1CC1c2cc(Cl)cc(F)c2C2(CC2)N1CC1CC2(CNC(=O)O2)C1] | 580 |
| 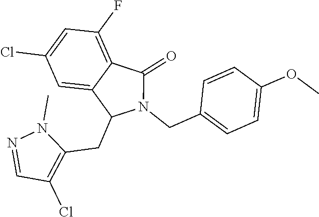 [SMILES: COc1ccc(CN2C(=O)c3c(F)cc(Cl)cc3C2Cc2c(Cl)cnn2C)cc1] | 581 |
| 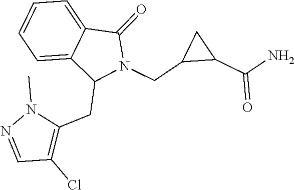 [SMILES: Cn1ncc(Cl)c1CC1c2ccccc2C(=O)N1CC1CC1C(N)=O] | 582 |
| 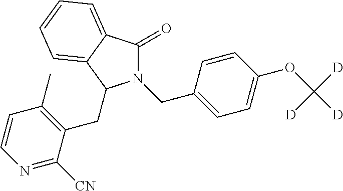 [SMILES: [2H]C([2H])([2H])Oc1ccc(CN2C(=O)c3ccccc3C2Cc2c(C)ccnc2C#N)cc1] | 583 |
| 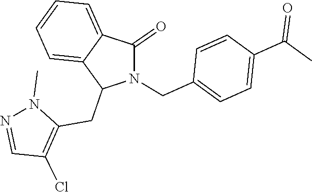 [SMILES: CC(=O)c1ccc(CN2C(=O)c3ccccc3C2Cc2c(Cl)cnn2C)cc1] | 584 |
| 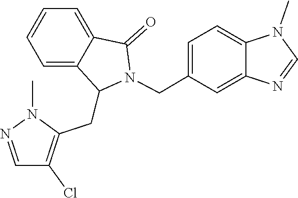 [SMILES: Cn1ncc(Cl)c1CC1c2ccccc2C(=O)N1Cc1ccc2c(c1)ncn2C] | 585 |
| 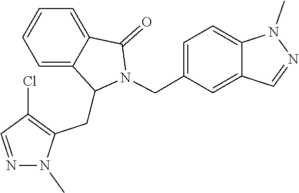 [SMILES: Cn1ncc(Cl)c1CC1c2ccccc2C(=O)N1Cc1ccc2c(cnn2C)c1] | 586 |
| 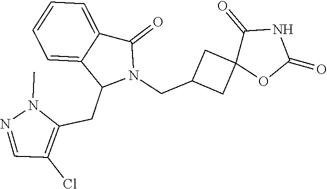 [SMILES: Cn1ncc(Cl)c1CC1c2ccccc2C(=O)N1CC1CC2(C1)OC(=O)NC2=O] | 587 |
| 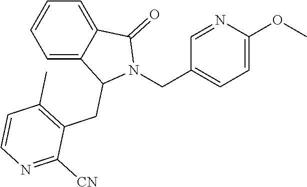 [SMILES: COc1ccc(CN2C(=O)c3ccccc3C2Cc2c(C)ccnc2C#N)cn1] | 588 |
| 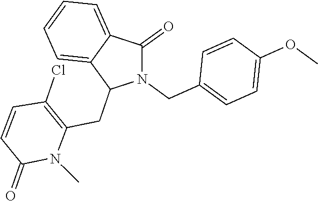 [SMILES: COc1ccc(CN2C(=O)c3ccccc3C2Cc2c(Cl)ccc(=O)n2C)cc1] | 589 |
| 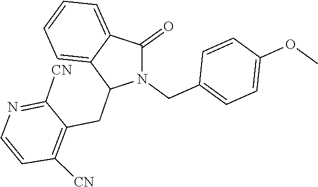 [SMILES: COc1ccc(CN2C(=O)c3ccccc3C2Cc2c(C#N)ccnc2C#N)cc1] | 590 |
| 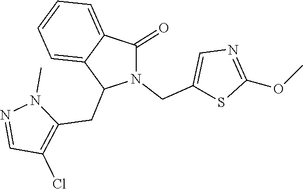 [SMILES: COc1ncc(CN2C(=O)c3ccccc3C2Cc2c(Cl)cnn2C)s1] | 591 |
| 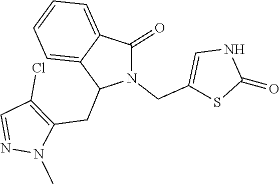 [SMILES: Cn1ncc(Cl)c1CC1c2ccccc2C(=O)N1Cc1c[nH]c(=O)s1] | 592 |
| 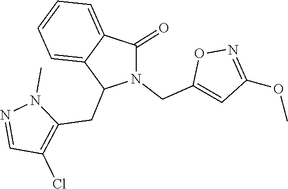 [SMILES: COc1cc(CN2C(=O)c3ccccc3C2Cc2c(Cl)cnn2C)on1] | 593 |
| 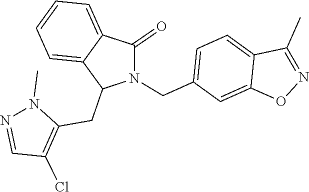 [SMILES: Cc1noc2cc(CN3C(=O)c4ccccc4C3Cc3c(Cl)cnn3C)ccc12] | 594 |
| 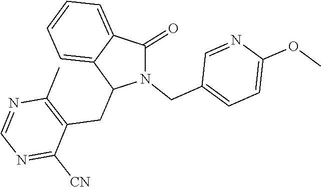 [SMILES: COc1ccc(CN2C(=O)c3ccccc3C2Cc2c(C)ncnc2C#N)cn1] | 595 |
| 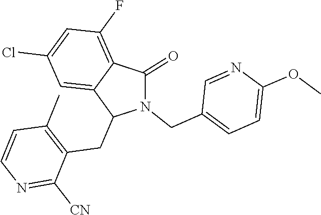 [SMILES: COc1ccc(CN2C(=O)c3c(F)cc(Cl)cc3C2Cc2c(C)ccnc2C#N)cn1] | 596 |
| 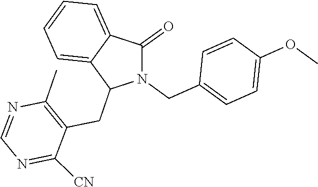 [SMILES: COc1ccc(CN2C(=O)c3ccccc3C2Cc2c(C)ncnc2C#N)cc1] | 597 |
| 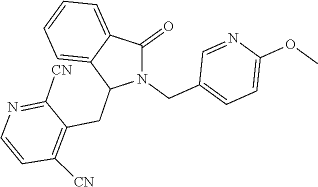 [SMILES: COc1ccc(CN2C(=O)c3ccccc3C2Cc2c(C#N)ccnc2C#N)cn1] | 598 |
| 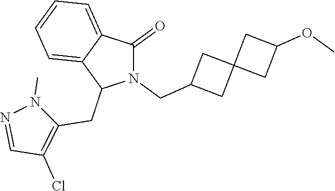 [SMILES: COC1CC2(CC(CN3C(=O)c4ccccc4C3Cc3c(Cl)cnn3C)C2)C1] | 599 |
| 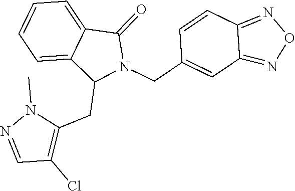 [SMILES: Cn1ncc(Cl)c1CC1c2ccccc2C(=O)N1Cc1ccc2nonc2c1] | 600 |
| 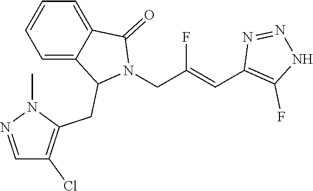 [SMILES: Cn1ncc(Cl)c1CC1c2ccccc2C(=O)N1C/C(F)=C/c1nn[nH]c1F] | 601 |
| 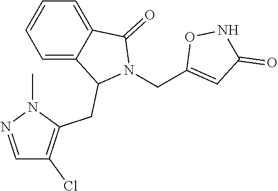 [SMILES: Cn1ncc(Cl)c1CC1c2ccccc2C(=O)N1Cc1cc(=O)[nH]o1] | 602 |
| 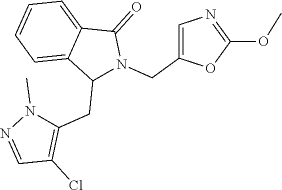 [SMILES: COc1ncc(CN2C(=O)c3ccccc3C2Cc2c(Cl)cnn2C)o1] | 603 |
| 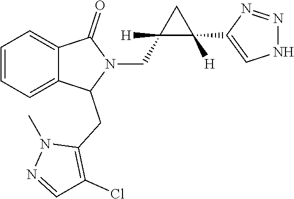 [SMILES: Cn1ncc(Cl)c1CC1c2ccccc2C(=O)N1C[C@@H]1C[C@@H]1c1c[nH]nn1] | 604 |
| 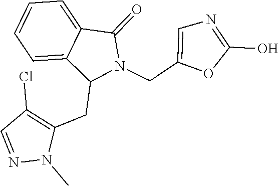 [SMILES: Cn1ncc(Cl)c1CC1c2ccccc2C(=O)N1Cc1cnc(O)o1] | 605 |
| 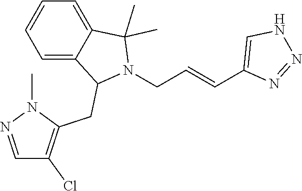 [SMILES: Cn1ncc(Cl)c1CC1c2ccccc2C(C)(C)N1C/C=C/c1c[nH]nn1] | 606 |
| 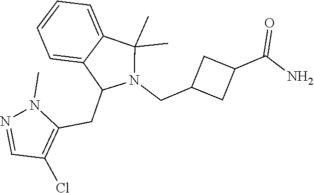 [SMILES: Cn1ncc(Cl)c1CC1c2ccccc2C(C)(C)N1CC1CC(C(N)=O)C1] | 607 |
| 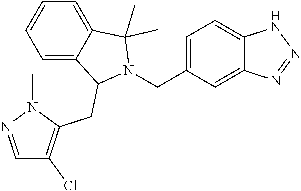 [SMILES: Cn1ncc(Cl)c1CC1c2ccccc2C(C)(C)N1Cc1ccc2[nH]nnc2c1] | 608 |
| 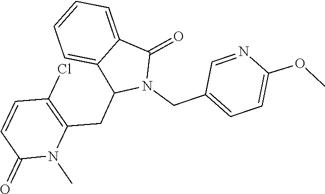 [SMILES: COc1ccc(CN2C(=O)c3ccccc3C2Cc2c(Cl)ccc(=O)n2C)cn1] | 609 |
| 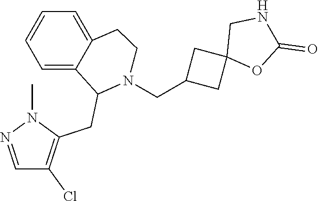 [SMILES: Cn1ncc(Cl)c1CC1c2ccccc2CCN1CC1CC2(CNC(=O)O2)C1] | 610 |
| 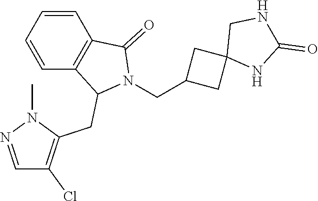 [SMILES: Cn1ncc(Cl)c1CC1c2ccccc2C(=O)N1CC1CC2(CNC(=O)N2)C1] | 611 |
| 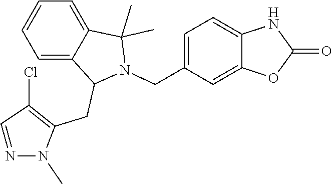 [SMILES: Cn1ncc(Cl)c1CC1c2ccccc2C(C)(C)N1Cc1ccc2[nH]c(=O)oc2c1] | 612 |
| 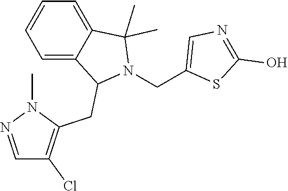 [SMILES: Cn1ncc(Cl)c1CC1c2ccccc2C(C)(C)N1Cc1cnc(O)s1] | 613 |
| 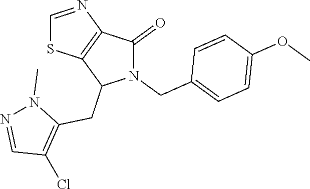 [SMILES: COc1ccc(CN2C(=O)c3ncsc3C2Cc2c(Cl)cnn2C)cc1] | 614 |
| 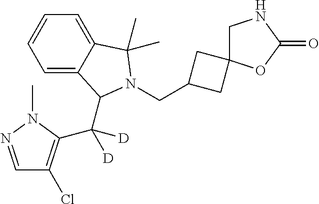 [SMILES: [2H]C([2H])(c1c(Cl)cnn1C)C1c2ccccc2C(C)(C)N1CC1CC2(CNC(=O)O2)C1] | 615 |
| 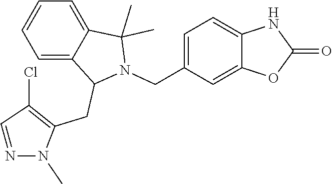 [SMILES: Cn1ncc(Cl)c1CC1c2ccccc2C(C)(C)N1Cc1ccc2[nH]c(=O)oc2c1] | 616 |
| 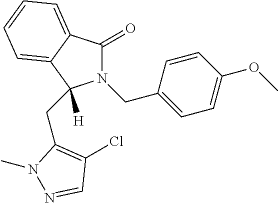 | 617 |
| 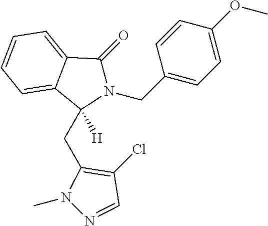 [SMILES: COc1ccc(CN2C(=O)c3ccccc3[C@@H]2Cc2c(Cl)cnn2C)cc1] | 618 |
| 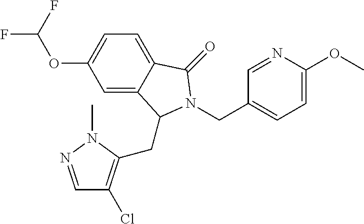 [SMILES: COc1ccc(CN2C(=O)c3ccc(OC(F)F)cc3C2Cc2c(Cl)cnn2C)cn1] | 619 |
| 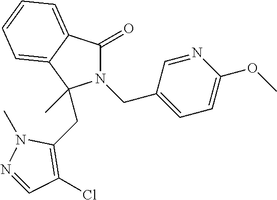 [SMILES: COc1ccc(CN2C(=O)c3ccccc3C2(C)Cc2c(Cl)cnn2C)cn1] | 620 |
| 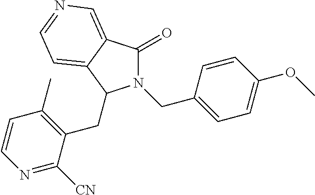 [SMILES: COc1ccc(CN2C(=O)c3cnccc3C2Cc2c(C)ccnc2C#N)cc1] | 621 |
| 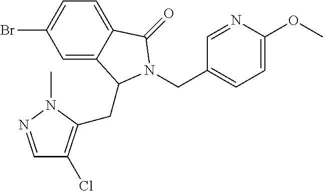 [SMILES: COc1ccc(CN2C(=O)c3ccc(Br)cc3C2Cc2c(Cl)cnn2C)cn1] | 622 |
| 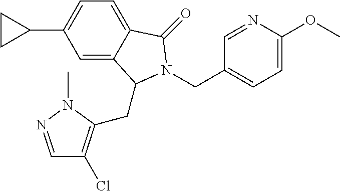 [SMILES: COc1ccc(CN2C(=O)c3ccc(C4CC4)cc3C2Cc2c(Cl)cnn2C)cn1] | 623 |
| 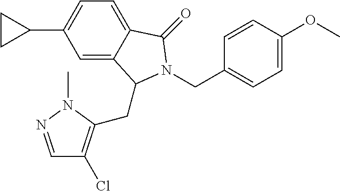 [SMILES: COc1ccc(CN2C(=O)c3ccc(C4CC4)cc3C2Cc2c(Cl)cnn2C)cc1] | 624 |
| 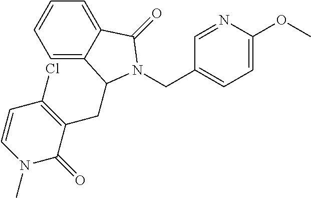 [SMILES: COc1ccc(CN2C(=O)c3ccccc3C2Cc2c(Cl)ccn(C)c2=O)cn1] | 625 |
| 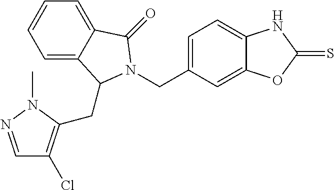 [SMILES: Cn1ncc(Cl)c1CC1c2ccccc2C(=O)N1Cc1ccc2[nH]c(=S)oc2c1] | 626 |
| 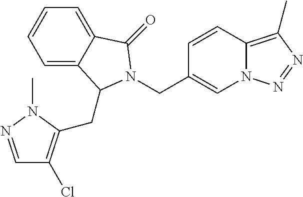 [SMILES: Cc1nnn2cc(CN3C(=O)c4ccccc4C3Cc3c(Cl)cnn3C)ccc12] | 627 |
| 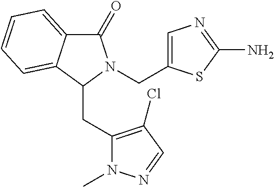 [SMILES: Cn1ncc(Cl)c1CC1c2ccccc2C(=O)N1Cc1cnc(N)s1] | 628 |
| 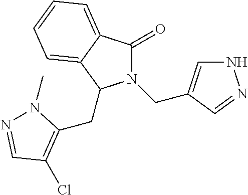 [SMILES: Cn1ncc(Cl)c1CC1c2ccccc2C(=O)N1Cc1cn[nH]c1] | 629 |
| 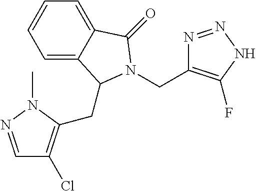 [SMILES: Cn1ncc(Cl)c1CC1c2ccccc2C(=O)N1Cc1nn[nH]c1F] | 630 |
| 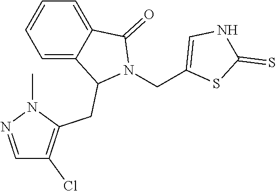 [SMILES: Cn1ncc(Cl)c1CC1c2ccccc2C(=O)N1Cc1c[nH]c(=S)s1] | 631 |
| 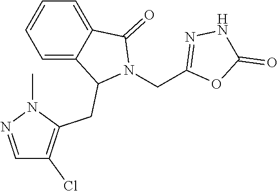 [SMILES: Cn1ncc(Cl)c1CC1c2ccccc2C(=O)N1Cc1n[nH]c(=O)o1] | 632 |
| 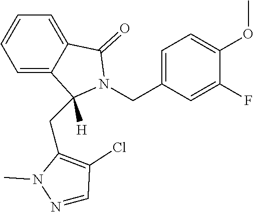 [SMILES: COc1ccc(CN2C(=O)c3ccccc3[C@H]2Cc2c(Cl)cnn2C)cc1F] | 633 |
| 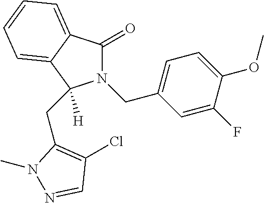 | 634 |
| 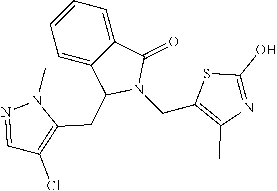 [SMILES: Cc1nc(O)sc1CN1C(=O)c2ccccc2C1Cc1c(Cl)cnn1C] | 635 |
| 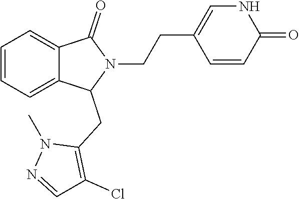 [SMILES: Cn1ncc(Cl)c1CC1c2ccccc2C(=O)N1CCc1ccc(=O)[nH]c1] | 636 |
| 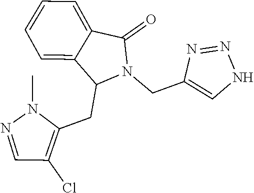 [SMILES: Cn1ncc(Cl)c1CC1c2ccccc2C(=O)N1Cc1c[nH]nn1] | 637 |
| 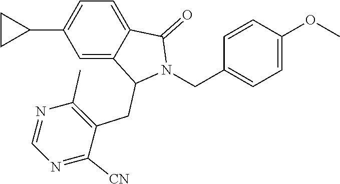 [SMILES: COc1ccc(CN2C(=O)c3ccc(C4CC4)cc3C2Cc2c(C)ncnc2C#N)cc1] | 638 |
| 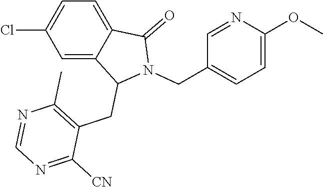 [SMILES: COc1ccc(CN2C(=O)c3ccc(Cl)cc3C2Cc2c(C)ncnc2C#N)cn1] | 639 |
| 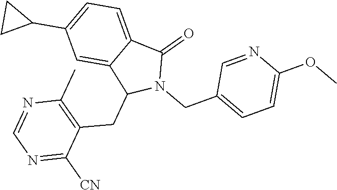 [SMILES: COc1ccc(CN2C(=O)c3ccc(C4CC4)cc3C2Cc2c(C)ncnc2C#N)cn1] | 640 |
| 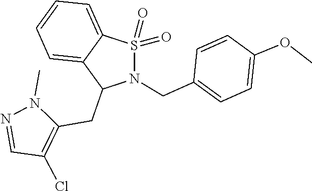 [SMILES: COc1ccc(CN2C(Cc3c(Cl)cnn3C)c3ccccc3S2(=O)=O)cc1] | 641 |
| 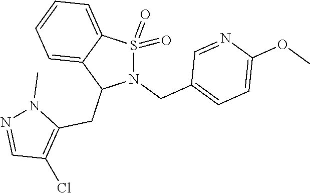 [SMILES: COc1ccc(CN2C(Cc3c(Cl)cnn3C)c3ccccc3S2(=O)=O)cn1] | 642 |
| 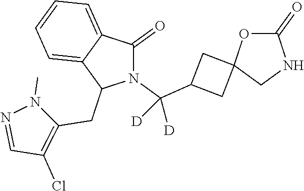 [SMILES: [2H]C([2H])(C1CC2(CNC(=O)O2)C1)N1C(=O)c2ccccc2C1Cc1c(Cl)cnn1C] | 643 |
| 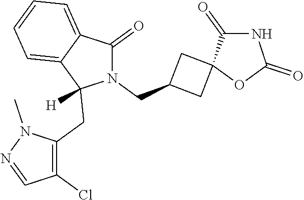 [SMILES: Cn1ncc(Cl)c1C[C@@H]1c2ccccc2C(=O)N1C[C@H]1C[C@]2(C1)OC(=O)NC2=O] | 644 |
| 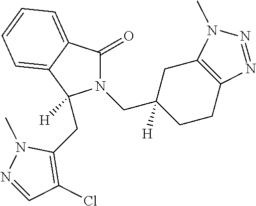 [SMILES: Cn1ncc(Cl)c1C[C@H]1c2ccccc2C(=O)N1C[C@@H]1CCc2nnn(C)c2C1] | 645 |

The notation “or 1” as used in chemical structures herein means that the stereo configuration of the chiral center labeled by “or 1” is not determined. For example, Compound 185 has an “or 1” positioned above the stereo center attached to a methyl group and a hydrogen atom. That means that the stereo configuration of the chiral carbon labeled by “or 1” is either R or S.

Another aspect of the disclosure provides a pharmaceutical composition comprising at least one compound selected from a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing can be administered as a separate composition concurrently with, prior to, or subsequent to, a composition comprising an additional active pharmaceutical agent.

In some embodiments, the pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

A compound selected from a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1)disclosed herein, a tautomer thereof, a solvate or stereoisomer of the compound or the tautomer, or a pharmaceutically acceptable salt of the foregoing, or a pharmaceutical composition disclosed herein can be administered orally in solid dosage forms, such as capsules, tablets, troches, dragées, granules and powders, or in liquid dosage forms, such as elixirs, syrups, emulsions, dispersions, and suspensions. The compound, tautomer, solvate, stereoisomer, or pharmaceutically acceptable salt described herein can also be administered parenterally, in sterile liquid dosage forms, such as dispersions, suspensions or solutions. Other dosages forms that can also be used to administer the compound, tautomer, solvate, stereoisomer, or pharmaceutically acceptable salt described herein as an ointment, cream, drops, transdermal patch or powder for topical administration, as an ophthalmic solution or suspension formation, e.g., eye drops, for ocular administration, as an aerosol spray or powder composition for inhalation or intranasal administration, or as a cream, ointment, spray or suppository for rectal or vaginal administration.

Gelatin capsules containing a compound, a tautomer thereof, a solvate or stereoisomer of the compound or the tautomer, and/or a pharmaceutically acceptable salt of the foregoing disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water-soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol.

For administration by inhalation, the compound, tautomer, solvate, stereoisomer, or pharmaceutically acceptable salt described herein may be conveniently delivered in the form of an aerosol spray presentation from pressurized packs or nebulisers. The compound, tautomer, solvate, stereoisomer, or pharmaceutically acceptable salt described herein may also be delivered as powders, which may be formulated, and the powder composition may be inhaled with the aid of an insufflation powder inhaler device. One exemplary delivery system for inhalation can be metered dose inhalation (MDI) aerosol, which may be formulated as a suspension or solution of a compound, tautomer, solvate, stereoisomer, or pharmaceutically acceptable salt described herein in at least one suitable propellant, selected, for example, from fluorocarbons and hydrocarbons.

For ocular administration, an ophthalmic preparation may be formulated with an appropriate weight percentage of a solution or suspension of the compound, tautomer, solvate, stereoisomer, or pharmaceutically acceptable salt described herein in an appropriate ophthalmic vehicle, such that the compound, tautomer, solvate, stereoisomer, or pharmaceutically acceptable salt described herein is maintained in contact with the ocular surface for a sufficient time period to allow the compound to penetrate the corneal and internal regions of the eye.

Useful pharmaceutical dosage-forms for administration of the compound, tautomer, solvate, stereoisomer, or pharmaceutically acceptable salt described herein include, but are not limited to, hard and soft gelatin capsules, tablets, parenteral injectables, and oral suspensions. In some embodiments, the pharmaceutical compositions disclosed herein may be in the form of controlled release or sustained release compositions as known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the active material is usually a component ranging from about 0.1 to about 50% by weight or preferably from about 1 to about 40% by weight with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

In some embodiments, unit capsules can be prepared by filling standard two-piece hard gelatin capsules each with, for example, 100 milligrams of the compound, tautomer, solvate, stereoisomer, or pharmaceutically acceptable salt described herein in powder, 150 milligrams of lactose, 50 milligrams of cellulose, and 6 milligrams magnesium stearate.

In some embodiments, a mixture of the compound, tautomer, solvate, stereoisomer, or pharmaceutically acceptable salt described herein and a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, tablets can be prepared by conventional procedures so that the dosage unit comprises, for example, 100 milligrams of the compound, stereoisomers thereof, or pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereoisomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, or pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, tautomer, solvate, stereoisomer, or pharmaceutically acceptable salt described herein is administered stepwise or in conjunction with at least one other therapeutic agent. When drugs are administered in physical combination, the dosage form and administration route should be selected depending on the compatibility of the combined drugs. Thus, the term coadministration is understood to include the administration of at least two agents concomitantly or sequentially, or alternatively as a fixed dose combination of the at least two active components.

The compound, tautomer, solvate, stereoisomer, or pharmaceutically acceptable salt disclosed herein can be administered as the sole active ingredient or in combination with at least one second active ingredient.

The compound, tautomer, solvate, or stereoisomer described herein may be used in the aforementioned form or in the form of their pharmaceutically acceptable salts, such as hydrochlorides, hydrobromides, acetates, sulfates, citrates, carbonates, trifluoroacetates and the like. When the compound, tautomer, solvate, stereoisomer, or pharmaceutically acceptable salt described herein contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When the compound, tautomer, solvate, or stereoisomer described herein contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts,” Journal of Pharmaceutical Science, 1977, 66, 1-19).

Neutral forms of the pharmaceutically acceptable salt described herein may be regenerated by contacting the salt with a base or acid, and isolating the parent compound in the conventional manner.

This disclosure provides prodrugs. Prodrugs of the compound, tautomer, solvate, stereoisomer, or pharmaceutically acceptable salt described herein that readily undergo chemical changes under physiological conditions to provide the compound, tautomer, solvate, stereoisomer, or pharmaceutically acceptable salt of the present disclosure. Additionally, prodrugs can be converted to the compound, tautomer, solvate, stereoisomer, or a pharmaceutically acceptable salt of the present disclosure by chemical or biochemical methods in an ex vivo environment. For example, prodrugs can be slowly converted to the compound, tautomer, solvate, stereoisomer, or pharmaceutically acceptable salt of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, i.e., the active entity.

Certain compound, tautomer, stereoisomer, or pharmaceutically acceptable salt of the disclosure can exist in unsolvated forms as well as solvated forms, including hydrate forms. Certain compound, tautomer, solvate, stereoisomer, or pharmaceutically acceptable salt of the disclosure may exist in multiple crystalline or amorphous forms.

Certain compound, tautomer, solvate, or pharmaceutically acceptable salt in this disclosure possesses asymmetric carbon atoms (optical centers) or double bonds; the racemates, enantiomers, diastereoisomers, geometric isomers and individual isomers are all intended to be encompassed within the scope of the present disclosure.

### III. Methods of Treatment and Uses

The present disclosure provides methods of treatment and uses utilizing a compound set forth in any one of the various embodiments of Section II (Compounds and Compositions) and Table 1, e.g., a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h, as well as Compounds 1 to 645 in Table 1, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

One aspect of the disclosure provides a method of treating a disease or condition, comprising administering to a subject in need thereof, a therapeutically effective amount of a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, wherein the disease or condition includes, but is not limited to, cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury. In some embodiments, the disease or condition can benefit from NAMPT activation.

In another aspect, disclosed herein is a compound, tautomer, solvate, stereoisomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1), a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for use as a medicament.

In another aspect, disclosed herein is use of a compound, tautomer, solvate, stereoisomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for treating a disease or condition that includes, but is not limited to, cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury. In some embodiments, the disease or condition can benefit from NAMPT activation.

In a further aspect of this disclosure, a compound, tautomer, solvate, stereoisomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1), a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in treating a disease or condition that includes, but is not limited to, cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury. In some embodiments, the disease or condition can benefit from NAMPT activation.

Another aspect of the disclosure provides a method of increasing NAD+ levels, comprising administering to a subject in need thereof, a therapeutically effective amount of a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for the manufacture of a medicament for increasing NAD+ levels.

In a further aspect of this disclosure, a compound, tautomer, solvate, stereoisomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in increasing NAD+ levels.

Another aspect of the disclosure provides a method of modulating, e.g., activating, NAMPT in a subject in need thereof, comprising administering to the subject, a therapeutically effective amount of a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt.

In another aspect, disclosed herein is use of a compound, tautomer, solvate, stereoisomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, for modulating, e.g., activating, NAMPT in a subject in need thereof.

In another aspect of this disclosure, a compound, tautomer, a solvate or stereoisomer of the compound or the tautomer, or pharmaceutically acceptable salt as described herein, including a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt, is for use in modulating, e.g., activating, NAMPT in a subject in need thereof by contacting the subject with the compound, tautomer, a solvate or stereoisomer of the compound or the tautomer, pharmaceutically acceptable salt, or pharmaceutical composition.

A compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered once daily, twice daily, or three times daily, for example, for the treatment of a disease or condition, that includes, but is not limited to, cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal 25 degeneration, carpal tunnel syndrome, Guillain-Barre syndrome, nerve damage, polio (poliomyelitis), and spinal cord injury. In some embodiments, the disease or condition can benefit from NAMPT activation.

A compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt may be administered, for example, various manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

The contacting is generally effected by administering to the subject an effective amount of one or more compounds, tautomers, solvates, stereoisomers, and pharmaceutically acceptable salt disclosed herein. Generally, administration is adjusted to achieve a therapeutic dosage of about 0.1 to 50 mg/kg, preferably 0.5 to 10 mg/kg, more preferably 1 to 10 mg/kg, though optimal dosages are compound specific, and generally empirically determined for each compound.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, 2 mg to 1500 mg or 5 mg to 1000 mg of a compound of Formulae 1, 2a, 2a′, 2b, 2c, 2c-1, 2c-2, 2d-2i, 3a, 3a-1 to 3a-16, 3b-3h, 4a-4h, 5a-5e, 6a-6h, 7a-7e, 8, and 9a-9h (e.g., Compounds 1 to 645 in Table 1) disclosed herein, a tautomer thereof, a solvate or stereoisomer of the compound or the tautomer, or a pharmaceutically acceptable salt of the foregoing, or a pharmaceutical composition comprising any of the compound, tautomer, solvate, stereoisomer, and pharmaceutically acceptable salt are administered once daily, twice daily, or three times daily. The compound, tautomer, solvate, stereoisomer, or pharmaceutically acceptable salt described herein is administered for morning/daytime dosing, with off period at night.

### IV. Examples

In order that the disclosure described herein may be more fully understood, the following examples are disclosed herein. It should be understood that these examples are for illustrative purposes only and are not to be construed as limiting this disclosure in any way.

### Example I. Synthesis of Exemplary Compounds

The compounds of the disclosure, selected from a compound of the Formulae depicted herein, a tautomer thereof, a solvate or stereoisomer of the compound or the tautomer, or a pharmaceutically acceptable salt of the foregoing, can be made according to standard chemical practices or as illustrated herein, including the following general synthetic procedures and specific synthetic schemes for Compounds 1 to 645 as representative examples of Formula 1.

### Preparation of Intermediates:

### 6-(bromomethyl)-3-((2-(trimethylsilyl)ethoxy)methyl)benzo[d]oxazol-2(3H)-one (Int 1)

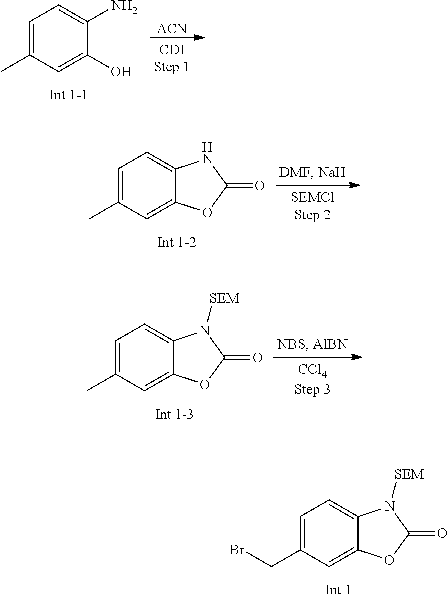

### Step 1. 6-methylbenzo[d]oxazol-2(3H)-one (Int 1-2)

To a solution of 2-amino-5-methylphenol (20 g, 162 mmol) in ACN (acetonitrile) (200 mL) was added CDI (N,N′-Carbonyldiimidazole) (52.67 g, 325 mmol) at r.t. The mixture was stirred for 14 h at 80° C. The mixture was diluted with EtOAc and washed with water. The organic layer was separated, dried over anhydrous Na₂SO₄ and concentrated under vacuum to give 6-methylbenzo[d]oxazol-2(3H)-one (22 g) as a light yellow solid, which was used in the next step without further purification. MS (ESI) m/z 150 [M+H]⁺.

### Step 2. 6-methyl-3-((2-(trimethylsilyl)ethoxy)methyl)benzo[d]oxazol-2(3H)-one (Int 1-3)

To a solution of 6-methylbenzo[d]oxazol-2(3H)-one (22 g, 148 mmol) in DMF (220 mL) was added NaH (3.89 g, 162 mmol) at 0° C. under N₂. The mixture was stirred for 30 min. SEM-C₁ (24.59 g, 148 mmol) was added and the reaction allowed to warm to r.t. This suspension was allowed to stir for an additional 2 h. Water was added at 0° C. and then extracted with EtOAc. The organic layer was separated, dried over anhydrous Na₂SO₄ and concentrated to give 6-methyl-3-(2-trimethylsilylethoxymethyl)-1,3-benzoxazol-2-one (35 g) as a yellow semi-solid, which was used in the next step without further purification. ¹H NMR (400 MHz, DMSO-d₆) δ 7.29-7.19 (m, 2H), 7.19-7.08 (m, 1H), 5.28 (s, 2H), 3.66 (t, J=7.9 Hz, 2H), 2.40 (s, 3H), 0.92 (t, J=7.9 Hz, 2H), 0.06 (s, 9H).

### Step 3. 6-(bromomethyl)-3-((2-(trimethylsilyl)ethoxy)methyl)benzo[d]oxazol-2(3H)-one (Int 1)

To a solution of 6-methyl-3-((2-(trimethylsilyl)ethoxy)methyl)benzo[d]oxazol-2(3H)-one (3 g, 10.7 mmol) in CCl₄ (30 mL) were added NBS (2.29 g, 12.9 mmol) and AIBN (176 mg, 1.07 mmol) at r.t. The mixture was stirred for 14 h at 80° C. After the reaction was completed, the solid was filtered, and the filtrate was concentrated. The residue was purified by silica gel flash column chromatography (PE/EtOAc=10/1) to give 6-(bromomethyl)-3-((2-(trimethylsilyl) ethoxy)methyl)benzo[d]oxazol-2(3H)-one (1.66 g, 43.2%) as a yellow semi-solid. ¹H NMR (400 MHz, CDCl₃) δ 7.31-7.25 (m, 2H), 7.17-7.08 (m, 1H), 5.28 (s, 2H), 4.56 (s, 2H), 3.65 (t, J=7.9 Hz, 2H), 0.95 (t, J=7.9 Hz, 2H), 0.06 (s, 9H).

### 6-((5-bromo-3-methyl-1-oxoisoindolin-2-yl)methyl)-3-((2-(trimethylsilyl)ethoxy)methyl) benzo[d]oxazol-2(3H)-one (Int 2) and 6-((5-bromo-1-methyl-3-oxoisoindolin-2-yl)methyl)-3-((2-(trimethylsilyl)ethoxy)methyl)benzo[d]oxazol-2(3H)-one (Int 3)

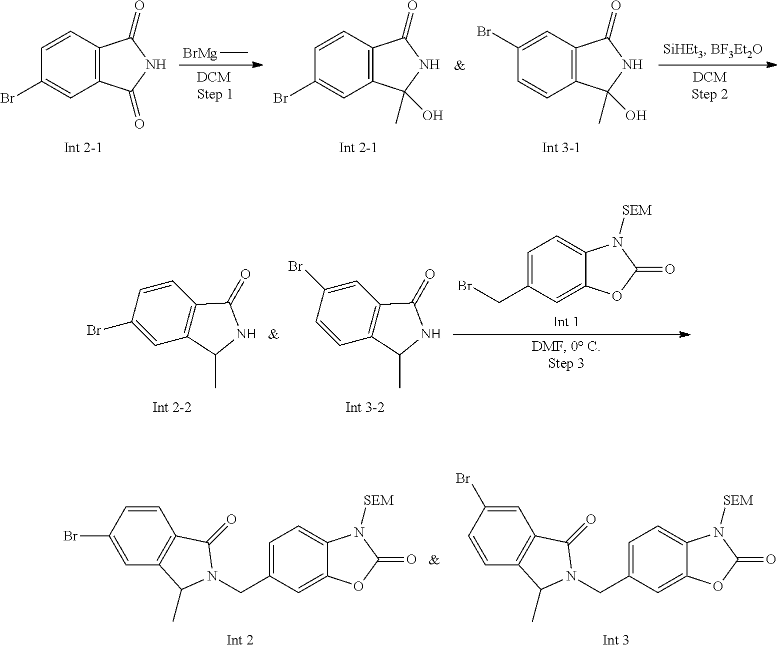

### Step 1. Mixture of 5-bromo-3-hydroxy-3-methylisoindolin-1-one (Int 2-1) and 6-bromo-3-hydroxy-3-methylisoindolin-1-one (Int 3-1)

To a solution of 5-bromoisoindoline-1,3-dione (15 g, 66.36 mmol) in DCM (300 mL) was added methylmagnesium bromide (1 M in THF, 200 mL) dropwise at 0° C. under N₂. After the addition, the mixture was stirred at 0° C. for 3 h before it was quenched with sat. NH₄Cl (aq.). After extraction with DCM, the organic layer was washed with brine, dried over anhydrous Na₂SO₄, filtered and concentrated in vacuo to give a crude product containing a mixture of 5-bromo-3-hydroxy-3-methylisoindolin-1-one and 6-bromo-3-hydroxy-3-methylisoindolin-1-one (16 g) as a white solid, which was used in the next step without further purification. MS (ESI) m/z 242 [M+H]⁺.

### Step 2. Mixture of 5-bromo-3-methylisoindolin-1-one (Int 2-2) and 6-bromo-3-methylisoindolin-1-one (Int 3-2)

Under N₂ protection, triethylsilane (30 g, 264 mmol) and boron trifluoride diethyl etherate (37.5 g, 264 mmol) were added successively at −15° C. to a mixture of 5-bromo-3-hydroxy-3-methylisoindolin-1-one and 6-bromo-3-hydroxy-3-methylisoindolin-1-one (16 g, 66 mmol) in dry DCM (160 mL). Afterwards, the reaction mixture was stirred at r.t. for 2 h and Sat. NaHCO₃(aq.) (100 mL) was added. The mixture was then extracted with DCM and the organic layer was washed with brine, dried over anhydrous Na₂SO₄, filtered and concentrated in vacuo. The residue was purified by silica gel flash column chromatography (PE/EtOAc=2/3) to give a mixture of 5-bromo-3-methylisoindolin-1-one and 6-bromo-3-methylisoindolin-1-one (11.5 g, 76%) as a white solid. MS (ESI) m/z 226 [M+H]⁺.

### Step 3. Mixture of 6-((5-bromo-3-methyl-1-oxoisoindolin-2-yl)methyl)-3-((2-(trimethylsilyl) ethoxy)methyl)benzo[d]oxazol-2(3H)-one (Int 2) and 6-((5-bromo-1-methyl-3-oxoisoindolin-2-yl)methyl)-3-((2-(trimethylsilyl)ethoxy)methyl)benzo[d]oxazol-2(3H)-one (Int 3)

To a solution of mixture of 5-bromo-3-methylisoindolin-1-one and 6-bromo-3-methylisoindolin-1-one (8 g, 35.3 mmol) in DMF (60 mL) was added NaH (934 mg, 38.9 mmol) at 0° C. under N₂. The mixture was stirred for 30 min. 6-(bromomethyl)-3-((2-(trimethylsilyl)ethoxy)methyl) benzo[d]oxazol-2(3H)-one (15.2 g, 42.4 mmol) was added and the reaction allowed to warm to r.t. This suspension was allowed to stir for an additional 2 h. Water was added at 0° C. and then extracted with EtOAc. The organic layer was separated, dried over anhydrous Na₂SO₄ and concentrated. The residue was purified by silica gel flash column chromatography (PE/EtOAc=3/1) to give a mixture of 6-((5-bromo-3-methyl-1-oxoisoindolin-2-yl)methyl)-3-((2-(trimethylsilyl)ethoxy)methyl)benzo[d]oxazol-2(3H)-one and 6-((5-bromo-1-methyl-3-oxoisoindolin-2-yl)methyl)-3-((2-(trimethylsilyl)ethoxy)methyl) benzo[d]oxazol-2(3H)-one (11 g, 21%) as a yellow semi-solid. MS (ESI) m/z 503 [M+H]⁺.

### 5-(bromomethyl)-1-((2-(trimethylsilyl)ethoxy)methyl)-1H-benzo[d][1,2,3]triazole (Int 4)

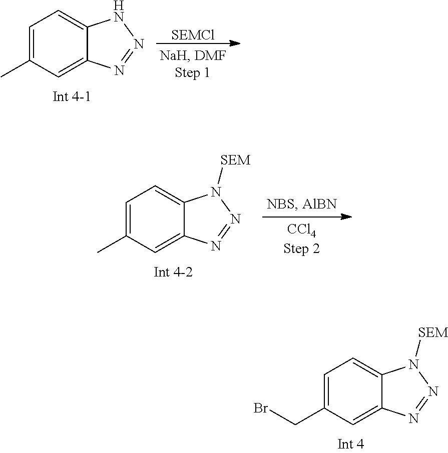

### Step 1. 5-methyl-1-((2-(trimethylsilyl)ethoxy)methyl)-1H-benzo[d][1,2,3]triazole (Int 4-2)

To a solution of 5-methyl-1H-benzo[d][1,2,3]triazole (40 g, 300 mmol) in DMF (400 mL) was added NaH (13.23 g, 330 mmol) portion-wise at 0° C. under N₂. The mixture was stirred for 30 min. (2-(chloromethoxy)ethyl) trimethylsilane (52.4 g, 315 mmol) was added and the reaction was warmed to r.t. This suspension was stirred for an additional 2 h. Water was added at 0° C. and then the mixture was extracted with EtOAc. The organic layer was separated, dried over anhydrous Na₂SO₄ and concentrated to give 5-methyl-1-((2-(trimethylsilyl)ethoxy)methyl)-1H-benzo[d][1,2,3]triazole (80 g) as a yellow oil, which was used in the next step without further purification. MS (ESI) m/z 264 [M+H]⁺.

### Step 2. 5-(bromomethyl)-1-((2-(trimethylsilyl)ethoxy)methyl)-1H-benzo[d][1,2,3]triazole (Int 4)

To a solution of 5-methyl-1-((2-(trimethylsilyl)ethoxy)methyl)-1H-benzo[d][1,2,3]triazole (80 g, 304 mmol) in CCl₄ (800 mL) were added NBS (59 g, 334 mmol) and AIBN (4.9 g, 30 mmol) at r.t. The mixture was stirred for 14 h at 80° C. After the reaction was completed, the solid was filtered, and the filtrate was concentrated. The residue was purified by silica gel flash column chromatography (PE/EtOAc=10/1) to afford 5-(bromomethyl)-1-((2-(trimethylsilyl)ethoxy) methyl)-1H-benzo[d][1,2,3]triazole (24 g, 23%) as a brown oil. MS (ESI) m/z 342 [M+H]⁺.

### 2-((1-((2-(trimethylsilyl)ethoxy)methyl)-1H-benzo[d][1,2,3]triazol-5-yl)methyl)isoindolin-1-one (Int 5)

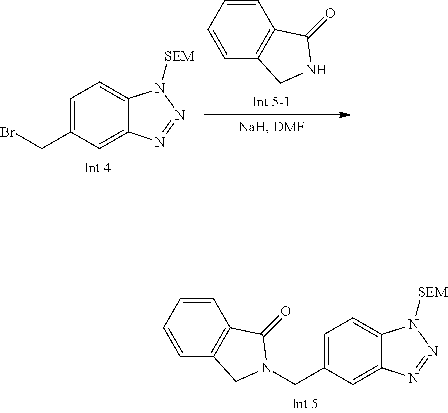 [SMILES: C[Si](C)(C)CCOCn1nnc2cc(CBr)ccc21 ; O=C1NCc2ccccc21 ; C[Si](C)(C)CCOCn1nnc2cc(CN3Cc4ccccc4C3=O)ccc21]

To a solution of isoindolin-1-one (7 g, 52 mmol) in DMF (70 mL) was added NaH (2.3 g, 57 mmol) at 0° C. under N₂. The mixture was stirred for 30 min. 5-(bromomethyl)-1-((2-(trimethylsilyl)ethoxy)methyl)-1H-benzo[d][1,2,3]triazole (24 g, 70 mmol) was added and the reaction was warmed to r.t. This suspension was stirred for an additional 2 h. Water was added at 0° C. and then the mixture was extracted with EtOAc. The organic layer was separated, dried over anhydrous Na₂SO₄ and concentrated under vacuum. The residue was purified by silica gel column chromatography (PE/EtOAc=3/2) to give 2-((1-((2-(trimethylsilyl) ethoxy)methyl)-1H-benzo[d][1,2,3]triazol-5-yl)methyl)isoindolin-1-one (10 g, 48%) as a brown oil. MS (ESI) m/z 395 [M+H]⁺.

### 6-((1-oxoisoindolin-2-yl)methyl)-3-((2-(trimethylsilyl)ethoxy)methyl)benzo[d]oxazol-2(3H)-one (Int 6), 6-((5-bromo-1-oxoisoindolin-2-yl)methyl)-3-((2-(trimethylsilyl)ethoxy) methyl)benzo[d]oxazol-2(3H)-one (Int 7) and 5-bromo-2-((1-((2-(trimethylsilyl)ethoxy) methyl)-1H-benzo[d][1,2,3]triazol-5-yl)methyl) isoindolin-1-one (Int 8)

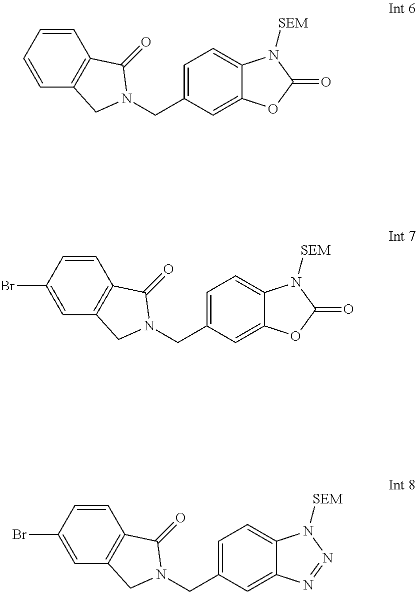 [SMILES: C[Si](C)(C)CCOCn1c(=O)oc2cc(CN3Cc4ccccc4C3=O)ccc21 ; C[Si](C)(C)CCOCn1c(=O)oc2cc(CN3Cc4cc(Br)ccc4C3=O)ccc21 ; C[Si](C)(C)CCOCn1nnc2cc(CN3Cc4cc(Br)ccc4C3=O)ccc21]

The Int 6, Int 7 and Int 8 were prepared according to the procedure described for Int 5. 6-(bromomethyl)-3-(4-methoxybenzyl)benzo[d]oxazol-2(3H)-one (Int 9)

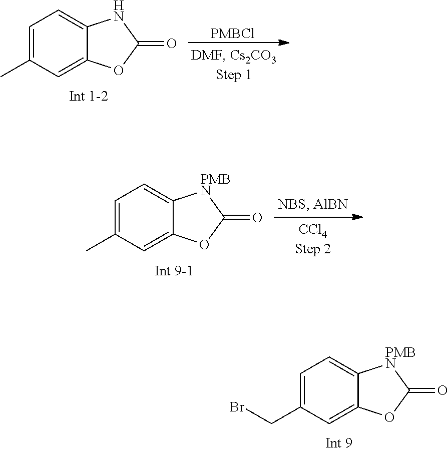

### Step 1. 3-(4-methoxybenzyl)-6-methylbenzo[d]oxazol-2(3H)-one (Int 9-1)

To a solution of 6-methylbenzo[d]oxazol-2(3H)-one (4 g, 26.8 mmol) in DMF (40 mL) were added Cs₂CO₃ (17.49 g, 53.6 mmol) and 1-(chloromethyl)-4-methoxybenzene (4.2 g, 26.8 mmol) at r.t. The mixture was stirred for 2 h at 60° C. The mixture was diluted with EtOAc and washed with water. The organic layer was separated, dried over anhydrous Na₂SO₄ and concentrated under vacuum to give 3-(4-methoxybenzyl)-6-methylbenzo[d]oxazol-2(3H)-one (6.5 g) as a yellow solid, which was used in the next step without further purification. MS (ESI) m/z 270 [M+H]⁺.

### Step 2. 6-(bromomethyl)-3-(4-methoxybenzyl)benzo[d]oxazol-2(3H)-one (Int 9)

To a solution of 3-(4-methoxybenzyl)-6-methylbenzo[d]oxazol-2(3H)-one (600 mg, 2.23 mmol) in CCl₄ (10 mL) were added NBS (436 mg, 2.45 mmol) and AIBN (37 mg, 0.22 mmol) at r.t. The mixture was stirred for 4 h at 80° C. After the reaction was completed, the solid was filtered, and the filtrate was concentrated. The residue was purified by silica gel flash column chromatography (PE/EtOAc=1/5) to afford 6-(bromomethyl)-3-(4-methoxybenzyl)benzo[d]oxazol-2(3H)-one (300 mg, 38.6%) as an off-white solid. MS (ESI) m/z 348 [M+H]⁺.

### (R)-6-((5-bromo-3-methyl-1-oxoisoindolin-2-yl)methyl)-3-((2-(trimethylsilyl)ethoxy) methyl)benzo[d]oxazol-2(3H)-one (Int 10) and (S)-6-((5-bromo-3-methyl-1-oxoisoindolin-2-yl)methyl)-3-((2-(trimethylsilyl)ethoxy)methyl)benzo[d]oxazol-2(3H)-one (Int 11)

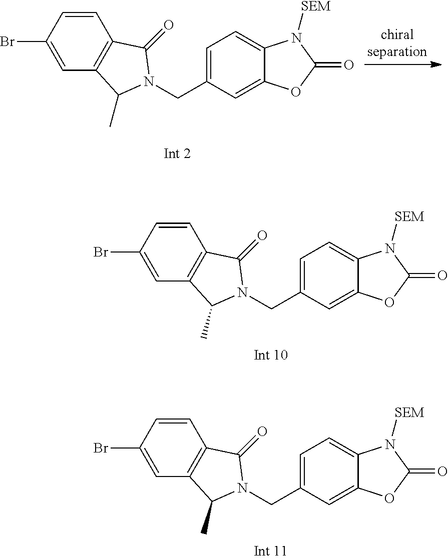 [SMILES: CC1c2cc(Br)ccc2C(=O)N1Cc1ccc2c(c1)oc(=O)n2COCC[Si](C)(C)C ; C[C@@H]1c2cc(Br)ccc2C(=O)N1Cc1ccc2c(c1)oc(=O)n2COCC[Si](C)(C)C ; C[C@H]1c2cc(Br)ccc2C(=O)N1Cc1ccc2c(c1)oc(=O)n2COCC[Si](C)(C)C]

Chiral separation of Int 2 by chiral SFC (column: CHIRALPAK AD-H; column size: 5 cm×25 cm, 5 μm; mobile phase A: CO₂; mobile phase B: MeOH (0.5% 2 mM NH₃-MeOH), flow rate: 200 g/min, wave length 220 nm) was conducted to afford two enantiomers: Int 10 and Int 11. One of the two enantiomers was a light yellow solid, with RT=1.728 min (column: Chiral ND (2) 3.0*100 mm, 3 μm; co-solvent: MeOH (0.1% DEA), flow: 2 ml/min; gradient: 10% to 50% in 2.0 min, hold 1.0 min at 50%; detector: 220 nm), MS (ESI) m/z 503/505 [M+H]⁺. The other one of the two enantiomers was a light yellow solid, with RT=1.969 min (column: Chiral ND (2) 3.0*100 mm, 3 μm; co-solvent: MeOH (0.1% DEA), flow: 2 ml/min; gradient: 10% to 50% in 2.0 min, hold 1.0 min at 50%; detector: 220 nm), MS (ESI) m/z 503/505 [M+H]⁺.

The intermediate Int 2-2 was prepared as follows:

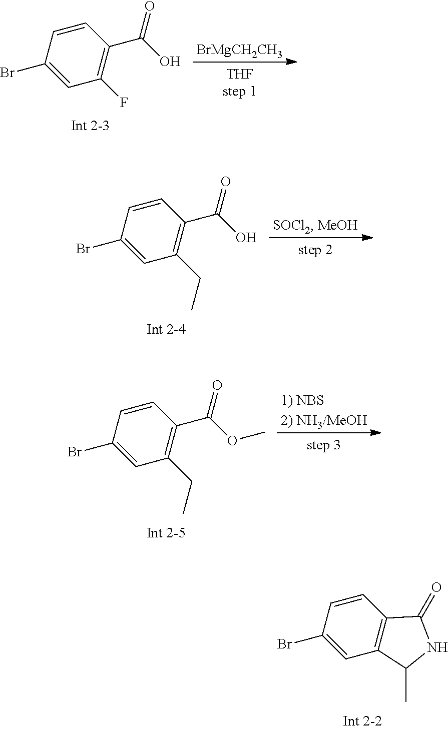

### Step 1. 4-bromo-2-ethylbenzoic acid (Int 2-4)

THF (2.5 L) and Int 2-3 (500 g, 2.28 mol) was charged into a 20 L reactor under N₂ protection. 2M ethyl magnesium bromide THF solution (2.5 L, 6.16 mol) was added to the above solution at 0° C. The mixture was stirred at r.t. for overnight. IPC with TLC showed the Int 2-3 was not detected. The mixture was charged into MeOH (1 L) at below 10° C. The mixture was concentrated. EA (5 L) and water (2.5 L) were charged into the mixture. 2M HCl (3.15 L) was added to the above solution at 15-30° C. After separation, aqueous phase was extracted with EA (2.5 L). The organic layers were combined, the mixture was charged into 5% Na₂CO₃ (5 L) at below 30° C., and then separated. Aqueous phase and EA (5000 mL) were charged into a 20 L reactor. 2M HCl (2.9 L) was added to the above solution at 15˜30° C., and then separated. The organic phase was washed 20% NaCl (2.5 L), and then dried with Na₂SO₄. After filtration, the filtrate was concentrated to afford the title compound as a white solid (231.3 g, 44%).

### Step 2. methyl 4-bromo-2-ethylbenzoate (Int 2-5)

MeOH (2 L) and Int 2-4 (200 g, 0.87 mol) were charged into a 2 L flask. SOC₂ (249.3 g, 2.10 mol) was added to the above solution at below 40° C. The mixture was stirred at 50-60° C. for overnight. IPC with TLC showed the Int 2-4 was not detected. The mixture was concentrated. EA (2 L) and water (2 L) were charged to the mixture. The mixture was charged into 5% Na₂CO₃ (100 mL) at below 30° C., and then separated. The organic phase was washed 20% NaCl (1 L). then dried with Na₂SO₄. After filtration, the filtrate was concentrated to afford the title compound as a white solid (214 g, 100%).

### Step 3: 5-bromo-3-methylisoindolin-1-one (Int 2-2)

CCl₄ (1 L), Int 2-5 (100 g, 0.41 mol), AIBN (13.5 g, 0.08 mol) and NBS (73 g, 0.41 mol) were charged into a 3 L flask at 15-30° C. The reactor was heated to 70˜80° C., and then stirred for 2 h. IPC with TLC showed the Int 2-5 was not detected. The mixture was filtered and the filtrate was collected and concentrated under vacuum. Then heptane (1 L) was charged into the reactor, and stirred for 15 min at 15˜30° C. After filtration, the filtrate was concentrated. Ammonia 2M solution in MeOH (1.32 L, 9.25 mol) were added to the above solution at 15˜30° C. The mixture was heated to 30˜35° C., and then stirred for overnight, concentrated under vacuum. Then EA (1 L) and PE (1 L) were charged into the reactor, and stirred for 15 min at 15-30° C. After filtration and drying for −2 h at 40-50° C., the title compound was obtained as a light yellow solid (117.2 g, 67%).

### 3-((3-bromopyridin-2-yl)methyl)isoindolin-1-one (Int 12)

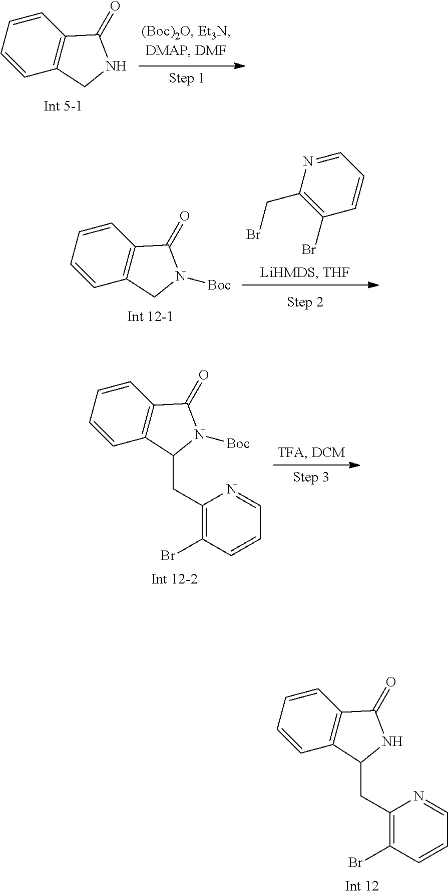 [SMILES: O=C1NCc2ccccc21 ; CC(C)(C)OC(=O)N1Cc2ccccc2C1=O ; BrCc1ncccc1Br ; CC(C)(C)OC(=O)N1C(=O)c2ccccc2C1Cc1ncccc1Br ; O=C1NC(Cc2ncccc2Br)c2ccccc21]

### Step 1. tert-butyl 1-oxoisoindoline-2-carboxylate (Int 12-1)

To a solution of Isoindolin-1-one (20 g, 150 mmol), (Boc)₂O (39.3 g, 180 mmol), Et₃N (45.5 g, 451 mmol) in THE (600 mL), was added DMAP (1.84 g, 15.0 mmol) at r.t. under N₂. After addition, the mixture was stirred at 25° C. for 1 h. LC-MS showed the starting material was mostly converted to the product. Then added water (200 mL) and extracted with EtOAc (100 mL×3). The organics were then combined and dried (Na₂SO₄) before concentration to dryness. The crude was purified with flash (PE/EtOAc=10:1) to give the product tert-butyl 1-oxoisoindoline-2-carboxylate (25 g, 71.4%) as a white solid. MS (ESI) m/z 234 [M+H]⁺.

### Step 2. tert-butyl 1-((3-bromopyridin-2-yl)methyl)-3-oxoisoindoline-2-carboxylate (12-2)

LiHMDS (99.6 ml, 99.6 mmol) was added to a solution of tert-butyl 1-oxoisoindoline-2-carboxylate (23.24 g, 99.6 mmol) in THF (100 mL) at −78° C. After stirring the solution at −78° C. for 1h, 3-bromo-2-(bromomethyl)pyridine (25.0 g, 99.6 mmol) in THE (100 mL) was added, then stirred at −78° C. for 1.5 h. LC-MS showed the starting material was mostly converted to the product. The reaction mixture was quenched with sat. NaCl (aq.), then extracted with EtOAc, and the combined organic layer was washed with sat. NaCl (aq.). The organic phase was dried over Na₂SO₄ and the solvent was evaporated. The residue was purified by flash-chromatography on silica gel with 25-40% EtOAc in hexane, to give the product tert-butyl 1-((3-bromopyridin-2-yl)methyl)-3-oxoisoindoline-2-carboxylate (15 g, 37.3%) as a white solid. MS (ESI) m/z 403 [M+H]⁺.

### Step 3. 3-((3-bromopyridin-2-yl)methyl)isoindolin-1-one (Int 12)

To a solution of tert-butyl 1-((3-bromopyridin-2-yl)methyl)-3-oxoisoindoline-2-carboxylate (15.0 g, 37.2 mmol) in DCM (16 mL) was added TFA (4 mL) and the mixture was stirred at r.t. for 2 h. The reaction progress was monitored by LC-MS. Once completed, the mixture was diluted with DCM (40 mL) and washed with sat. NaHCO₃ solution. The organic layer was dried over MgSO₄, filtered and concentrated under reduced pressure. The crude product was obtained as a black solid (15 g, 80.4%). MS (ESI) m/z 303 [M+H]⁺.

Final product HPLC condition: (column: XBridge® BEH Shield RP18; column size: 4.6*50 mm, 2.5 μm; Mobile Phase A: water with 0.10% FA, Mobile Phase B: ACN with 0.05% FA; flow: 1.5 mL/min; Gradient: 5% B to 95% B in 10 min). Final product yield was the yield of the last step.

### General Synthetic Procedures:

### Procedure A:

### 2-(4-hydroxybenzyl)isoquinolin-1(2H)-one (1)

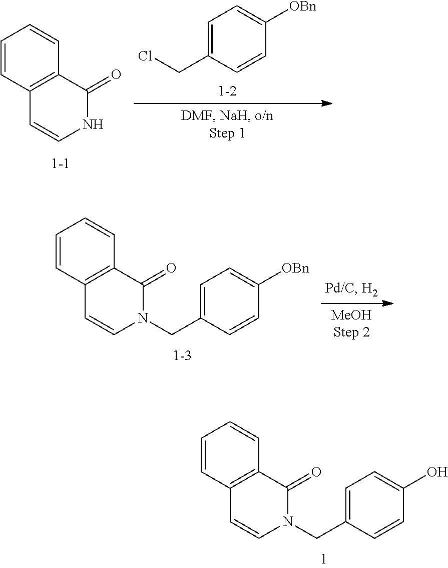

### Step 1. 2-(4-(benzyloxy)benzyl)isoquinolin-1(2H)-one (1-3)

To a solution of isoquinolin-1(2H)-one (150 mg, 1.03 mmol) in DMF (1.5 mL) was added NaH (25 mg, 1.03 mmol) at 0° C. under N₂. The mixture was stirred for 30 min. 1-(benzyloxy)-4-(chloromethyl)benzene (265 mg, 1.14 mmol) was added and the reaction allowed to warm to r.t. This suspension was allowed to stir for an additional 2 h. Water was added at 0° C. and then extracted with EtOAc. The organic layer was separated, dried over anhydrous Na₂SO₄ and concentrated under vacuum. The residue was purified by pre-TLC (PE/EtOAc=1.5/1) to give 2-(4-(benzyloxy)benzyl) isoquinolin-1(2H)-one (300 mg, 85%) as a white solid. MS (ESI) m/z 342 [M+H]⁺.

### Step 2. 2-(4-hydroxybenzyl)isoquinolin-1(2H)-one (1)

To a solution of 2-(4-(benzyloxy)benzyl)isoquinolin-1(2H)-one (300 mg, 0.88 mmol) in methanol (10 mL) in a 100 mL round bottom flask equipped with a magnetic stir bar and nitrogen inlet was added Pd/C (10 mg). The flask was evacuated and purged with nitrogen three times, and then evacuated and placed under hydrogen (1 atm). The mixture was stirred at r.t. overnight, filtered through a pad of Celite, and the filter cake washed with methanol. The filtrate was concentrated and purified by prep HPLC to give 2-(4-hydroxybenzyl)isoquinolin-1(2H)-one (49 mg, 22%) as an off-white solid. MS (ESI) m/z 252 [M+H]⁺. ¹H NMR (400 MHz, DMSO-d₆) b 9.40 (s, 1H), 8.23 (d, J=8.0 Hz, 1H), 7.73-7.66 (m, 1H), 7.64 (d, J=7.8 Hz, 1H), 7.55-7.46 (m, 2H), 7.18 (d, J=8.3 Hz, 2H), 6.71 (d, J=8.3 Hz, 2H), 6.62 (d, J=7.3 Hz, 1H), 5.05 (s, 2H).

### Procedure B:

### 6-((1-oxoisoquinolin-2(1H)-yl)methyl)benzo[d]oxazol-2(3H)-one (4)

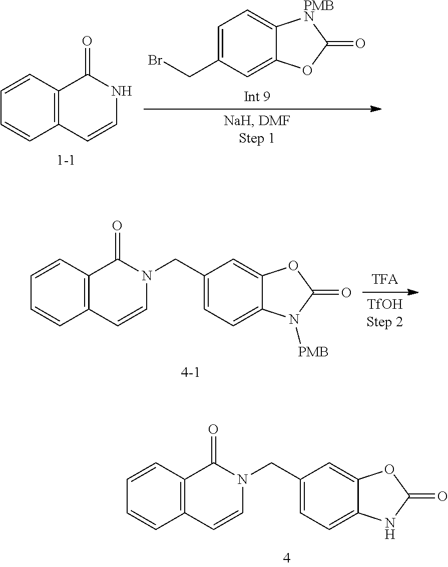

### Step 1. 3-(4-methoxybenzyl)-6-((1-oxoisoquinolin-2(1H)-yl)methyl)benzo[d]oxazol-2(3H)-one (4-1)

To a solution of isoquinolin-1(2H)-one (63 mg, 0.43 mmol) in DMF (1 mL) was added NaH (10 mg, 0.43 mmol) at 0° C. under N₂. The mixture was stirred for 30 min. 6-(bromomethyl)-3-(4-methoxybenzyl)benzo[d]oxazol-2(3H)-one (151 mg, 0.43 mmol) was added and the reaction allowed to warm to r.t. This suspension was allowed to stir for an additional 2 h. The mixture was diluted with EtOAc and washed with water. The organic layer was separated, dried over anhydrous Na₂SO₄ and concentrated under vacuum. The residue was purified by prep TLC (PE/EtOAc=1/1) to give 3-(4-methoxybenzyl)-6-((1-oxoisoquinolin-2(1H)-yl)methyl) benzo[d]oxazol-2(3H)-one (50 mg, 28%) as a yellow solid. MS (ESI) m/z 413 [M+H]⁺.

### Step 2. 6-((1-oxoisoquinolin-2(1H)-yl)methyl)benzo[d]oxazol-2(3H)-one (4)

TfOH (0.10 mL) was added to a stirred solution of 3-(4-methoxybenzyl)-6-((1-oxoisoquinolin-2(1H)-yl)methyl)benzo[d]oxazol-2(3H)-one (50 mg, 0.12 mmol) in TFA (1 mL). The mixture was stirred at 70° C. for 0.5 h. The mixture was concentrated in vacuo and purified by prep-HPLC to give 6-((1-oxoisoquinolin-2(1H)-yl)methyl)benzo[d]oxazol-2(3H)-one (12 mg, 34%) as a white solid. MS (ESI) m/z 293 [M+H]⁺. ¹H NMR (400 MHz, DMSO-d₆) δ 11.61 (s, 1H), 8.23 (d, J=8.0 Hz, 1H), 7.75-7.68 (m, 1H), 7.65 (d, J=7.8 Hz, 1H), 7.59 (d, J=7.4 Hz, 1H), 7.51 (t, J=7.5 Hz, 1H), 7.33 (s, 1H), 7.16 (d, J=8.0 Hz, 1H), 7.04 (d, J=8.0 Hz, 1H), 6.65 (d, J=7.4 Hz, 1H), 5.17 (s, 2H).

### Procedure C:

### 2-((1H-benzo[d][1,2,3]triazol-5-yl)methyl)isoquinolin-1(2H)-one (7)

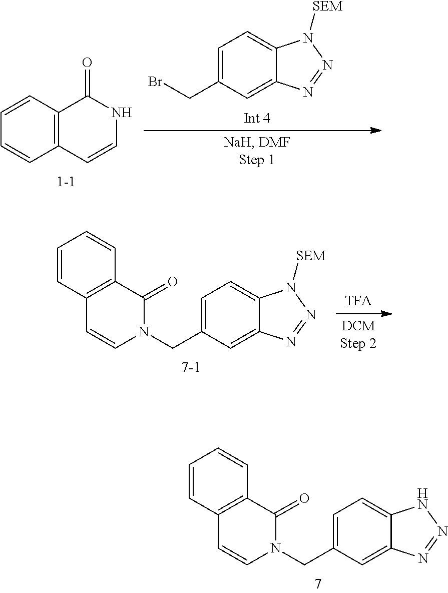 [SMILES: O=c1[nH]ccc2ccccc12 ; C[Si](C)(C)CCOCn1nnc2cc(CBr)ccc21 ; C[Si](C)(C)CCOCn1nnc2cc(Cn3ccc4ccccc4c3=O)ccc21 ; O=c1c2ccccc2ccn1Cc1ccc2[nH]nnc2c1]

### Step 1. 2-((1-((2-(trimethylsilyl)ethoxy)methyl)-1H-benzo[d][1,2,3]triazol-5-yl)methyl) isoquinolin-1(2H)-one (7-1)

To a solution of isoquinolin-1(2H)-one (50 mg, 0.34 mmol) in DMF (1 mL) was added NaH (8 mg, 0.34 mmol) at 0′C under N₂. The mixture was stirred for 30 min. 5-(bromomethyl)-1-((2-(trimethylsilyl)ethoxy)methyl)-1H-benzo[d][1,2,3]triazole (118 mg, 0.34 mmol) was added and the reaction allowed to warm to r.t. This suspension was allowed to stir for an additional 2 h. Water was added at 0′C and then extracted with EtOAc. The organic layer was separated, dried over anhydrous Na₂SO₄ and concentrated under vacuum. The residue was purified by prep TLC (PE/EtOAc=1.5/1) to give 2-((1-((2-(trimethylsilyl)ethoxy)methyl)-1H-benzo[d][1,2,3]triazol-5-yl) methyl)isoquinolin-1(2H)-one (50 mg, 35%) as a yellow solid. MS (ESI) m/z 407 [M+H]⁺.

### Step 2. 2-((1H-benzo[d][1,2,3]triazol-5-yl)methyl)isoquinolin-1(2H)-one (7)

TFA (1 mL) was added to a stirred solution of 2-((1-((2-(trimethylsilyl)ethoxy)methyl)-1H-benzo[d][1,2,3]triazol-5-yl)methyl)isoquinolin-1(2H)-one (50 mg, 0.12 mmol) in DCM (1 mL). The mixture was stirred for 5 h. The mixture was concentrated in vacuo and purified by C18 column (ACN=45%) to give 2-((1H-benzo[d][1,2,3]triazol-5-yl)methyl)isoquinolin-1(2H)-one (20 mg, 58%) as a white solid. MS (ESI) m/z 277 [M+H]⁺. ¹H NMR (400 MHz, DMSO-d₆) δ 15.58 (s, 1H), 8.25 (d, J=8.0 Hz, 1H), 7.89 (d, J=8.6 Hz, 1H), 7.81 (s, 1H), 7.74-7.63 (m, 3H), 7.52 (t, J=7.5 Hz, 1H), 7.45 (d, J=8.6 Hz, 1H), 6.68 (d, J=7.3 Hz, 1H), 5.36 (s, 2H).

### Procedure D:

### 2-(4-hydroxybenzyl)-5-methylisoindoline-1,3-dione (14)

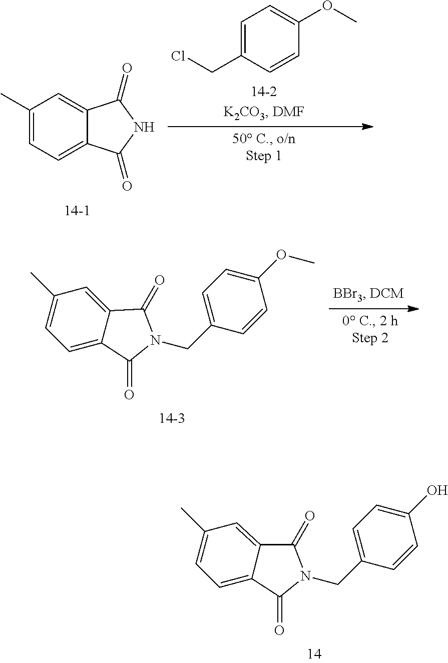

### Step 1. 2-(4-methoxybenzyl)-5-methylisoindoline-1,3-dione (14-3)

To a stirred solution of 5-methylisoindoline-1,3-dione (100 mg, 0.62 mmol) in DMF (5 mL) were added 1-(chloromethyl)-4-methoxybenzene (98 mg, 0.62 mmol) and K₂CO₃ (171 mg, 1.24 mmol). The reaction mixture was stirred at 50° C. overnight. The reaction mixture was quenched with water, extracted with EA (3×50 mL). The combined organic phase was washed with brine, dried over anhydrous Na₂SO₄ and concentrated to afford the title compound (174 mg, crude) as a light yellow solid. MS (ESI) m/z 282 [M+H]⁺.

### Step 2. 2-(4-hydroxybenzyl)-5-methylisoindoline-1,3-dione (14)

To a solution of 2-(4-methoxybenzyl)-5-methylisoindoline-1,3-dione (174 mg, 0.62 mmol) in DCM (5 mL) was added BBr₃ (466 mg, 1.86 mmol) at 0° C. for 2h, and then quenched with water, extracted with DCM (3×50 mL). The combined organic phase was washed with brine, dried over anhydrous Na₂SO₄ and concentrated. The residue was purified by Prep-HPLC to afford 2-(4-hydroxybenzyl)-5-methylisoindoline-1,3-dione (90 mg, 54.3%) as a white solid. MS (ESI) m/z 268 [M+H]⁺. ¹H NMR (400 MHz, DMSO-d₆) δ 11.64 (s, 1H), 7.61 (d, J=7.4 Hz, 1H), 7.53-7.48 (m, 1H), 7.43 (t, J=7.2 Hz, 1H), 7.28-7.18 (m, 2H), 7.17 (s, 1H), 7.11-6.97 (m, 5H), 5.18 (d, J=15.2 Hz, 1H), 4.71 (dd, J=6.4, 4.4 Hz, 1H), 4.44 (d, J=15.2 Hz, 1H), 3.45 (dd, J=14.0, 4.4 Hz, 1H), 3.14 (dd, J=14.0, 6.4 Hz, 1H).

### Procedure E:

### 6-((1-(2-fluorobenzyl)-3-oxoisoindolin-2-yl)methyl)benzo[d]oxazol-2(3H)-one (146)

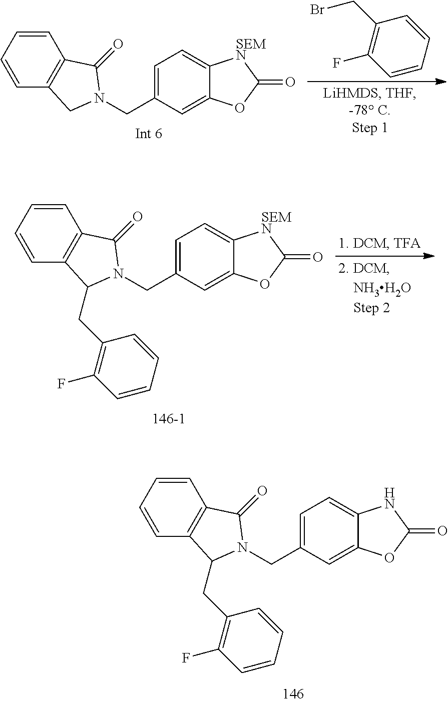 [SMILES: C[Si](C)(C)CCOCn1c(=O)oc2cc(CN3Cc4ccccc4C3=O)ccc21 ; Fc1ccccc1CBr ; C[Si](C)(C)CCOCn1c(=O)oc2cc(CN3C(=O)c4ccccc4C3Cc3ccccc3F)ccc21 ; O=C1c2ccccc2C(Cc2ccccc2F)N1Cc1ccc2[nH]c(=O)oc2c1]

### Step 1. 6-((1-(2-fluorobenzyl)-3-oxoisoindolin-2-yl)methyl)-3-((2-(trimethylsilyl) ethoxy) methyl)benzo[d]oxazol-2(3H)-one (146-1)

To a solution of 6-((1-oxoisoindolin-2-yl)methyl)-3-((2-(trimethylsilyl)ethoxy)methyl) benzo[d]oxazol-2(3H)-one (200 mg, 0.48 mmol) in THF (5 mL) was added LiHMDS (0.53 mL, 0.53 mmol) at −78° C. under N₂. The mixture was stirred for 30 min. Then 1-(bromomethyl)-2-fluorobenzene (92 mg, 0.48 mmol) was added. This suspension was allowed to stir for an additional 1 h. NH₄C₁(aq.) was added and then extracted with EtOAc. The organic layer was separated, dried over anhydrous Na₂SO₄ and concentrated under vacuum. The residue was purified by prep TLC (DCM/MeOH=25/1) to give the title compound (150 mg, 59%) as a yellow solid. MS (ESI) m/z 519 [M+H]⁺.

### Step 2. 6-((1-(2-fluorobenzyl)-3-oxoisoindolin-2-yl)methyl)benzo[d]oxazol-2(3H)-one (146)

To a solution of 6-((1-(2-fluorobenzyl)-3-oxoisoindolin-2-yl)methyl)-3-((2-(trimethylsilyl) ethoxy)methyl)benzo[d]oxazol-2(3H)-one (150 mg, 0.28 mmol) in DCM (3 mL) was added TFA (3 mL). The mixture was stirred for 3 h and then concentrated under vacuum. The residue was taken in DCM (3 mL) and ammonium hydroxide (1 mL) was added at 0′C. This suspension was allowed to stir for an additional 1 h. The mixture was concentrated and purified by prep HPLC to give the title compound (33 mg, 28%) as a white solid. MS (ESI) m/z 389 [M+H]⁺. ¹H NMR (400 MHz, DMSO-d₆) δ 8.58 (dd, J=4.8, 1.6 Hz, 1H), 7.92 (dd, J=6.4, 2.4 Hz, 1H), 7.88 (dd, J=8.0, 1.6 Hz, 1H), 7.59 (s, 1H), 7.53-7.47 (m, 2H), 7.35 (s, 1H), 7.24-7.14 (m, 2H), 5.30 (t, J=6.4 Hz, 1H), 5.11 (d, J=15.6 Hz, 11H), 4.43 (d, J=15.6 Hz, 11H), 3.47 (dd, J=15.4, 7.2 Hz, 1H), 3.35 (dd, J=15.4, 5.6 Hz, 1H).

### General Procedure F

### 6-((1-((3-bromopyridin-2-yl)methyl)-3-oxoisoindolin-2-yl)methyl)oxazolo[4,5-b]pyridin-2(3H)-one (222)

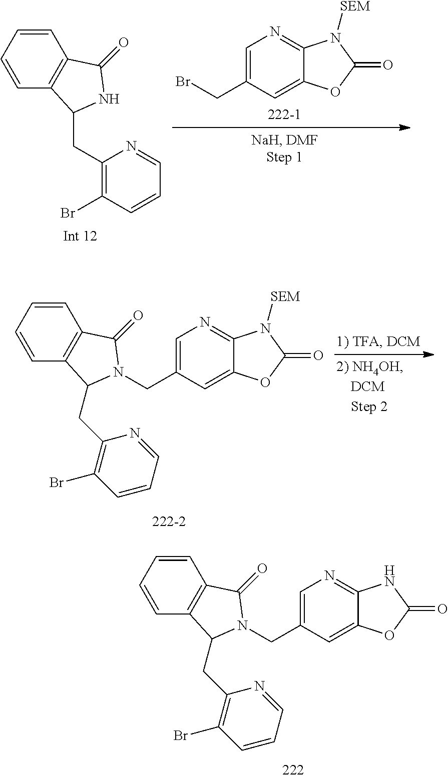 [SMILES: O=C1NC(Cc2ncccc2Br)c2ccccc21 ; C[Si](C)(C)CCOCn1c(=O)oc2cc(CBr)cnc21 ; C[Si](C)(C)CCOCn1c(=O)oc2cc(CN3C(=O)c4ccccc4C3Cc3ncccc3Br)cnc21 ; O=C1c2ccccc2C(Cc2ncccc2Br)N1Cc1cnc2[nH]c(=O)oc2c1]

### Step 1. 6-((1-((3-bromopyridin-2-yl)methyl)-3-oxoisoindolin-2-yl)methyl)-3-((2-(trimethylsilyl)ethoxy)methyl)oxazolo[4,5-b]pyridin-2(3H)-one (222-2)

To a solution of 3-((3-bromopyridin-2-yl)methyl)isoindolin-1-one (17 mg, 0.06 mmol) in DMF (1 mL), was added NaH (3 mg, 0.07 mmol) at 0° C. under N₂. After addition, the mixture was stirred at 0° C. for 1 h. Then added 6-(bromomethyl)-3-(2-trimethylsilylethoxymethyl)oxazolo[4,5-b]pyridin-2-one (20 mg, 0.06 mmol) at 0° C. under N₂. After addition, the mixture was stirred at 25° C. for 1 h. LC-MS showed the starting material was mostly converted to the product. Then added water (10 mL) and extracted with EtOAc (10 mL×3). The organics were then combined and dried (Na₂SO₄) before concentration to dryness. The crude was purified by flash (PE/EtOAc=1:1) to give 6-((1-((3-bromopyridin-2-yl)methyl)-3-oxoisoindolin-2-yl)methyl)-3-((2-(trimethylsilyl)ethoxy)methyl)oxazolo[4,5-b]pyridin-2(3H)-one (25 mg, 77.2%) as a white solid. MS (ESI) m/z 581 [M+H]⁺.

### Step 2. 6-((1-((3-bromopyridin-2-yl)methyl)-3-oxoisoindolin-2-yl)methyl)oxazolo[4,5-b]pyridin-2(3H)-one (222)

To a solution of 6-((1-((3-bromopyridin-2-yl)methyl)-3-oxoisoindolin-2-yl)methyl)-3-((2-(trimethylsilyl)ethoxy)methyl)oxazolo[4,5-b]pyridin-2(3H)-one (20 mg, 0.03 mmol) in DCM (1 mL) was added TFA (1 mL). The mixture was stirred for 1 h and then concentrated under vacuum. The residue was taken in DCM (1 mL) and ammonium hydroxide (1 mL) was added at 0° C. This suspension was allowed to stir for an additional 1 h. The mixture was concentrated and purified by prep HPLC to give 6-((1-((3-bromopyridin-2-yl)methyl)-3-oxoisoindolin-2-yl)methyl)oxazolo[4,5-b]pyridin-2(3H)-one (4 mg, 24.5%) as a white solid. MS (ESI) m/z 451 [M+H]⁺. ¹H NMR (400 MHz, CDCl₃) δ 8.58 (dd, J=4.8, 1.6 Hz, 1H), 7.92 (dd, J=6.4, 2.4 Hz, 1H), 7.88 (dd, J=8.0, 1.6 Hz, 1H), 7.59 (s, 1H), 7.53-7.47 (m, 2H), 7.35 (s, 1H), 7.24-7.14 (m, 2H), 5.30 (t, J=6.4 Hz, 1H), 5.11 (d, J=15.6 Hz, 1H), 4.43 (d, J=15.6 Hz, 1H), 3.47 (dd, J=15.4, 7.2 Hz, 1H), 3.35 (dd, J=15.4, 5.6 Hz, 1H).

The intermediate 222-1 was prepared as follows:

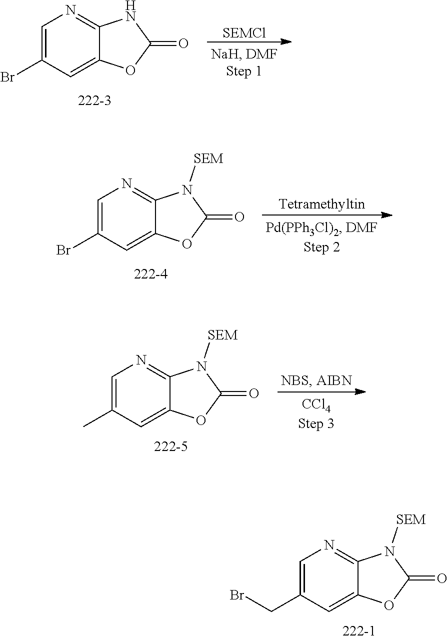

### Step 1. 6-bromo-3-((2-(trimethylsilyl)ethoxy)methyl)oxazolo[4,5-b]pyridin-2(3H)-one (222-4)

To a solution of 6-bromo-3H-oxazolo[4,5-b]pyridin-2-one (4.3 g, 20 mmol) in DMF (40 mL), was added NaH (959 mg, 24 mmol) at 0° C. under N₂. After addition, the mixture was stirred at 0° C. for 1 h. Then SEMCl (3.67 g, 22 mmol) was added, and the mixture was stirred at 0° C. for 1 h. LC-MS showed the starting material was mostly converted to the product. Then added water (100 mL) and extracted with EtOAc (50 mL×3). The organics were combined and dried (Na₂SO₄) before concentration to dryness. The crude was purified by flash (PE/EtOAc=5:1) to give 6-bromo-3-((2-(trimethylsilyl)ethoxy)methyl)oxazolo[4,5-b]pyridin-2(3H)-one (5 g, 68.9%) as a yellow oil. MS (ESI) m/z 345 [M+H]⁺.

### Step 2. 6-methyl-3-((2-(trimethylsilyl)ethoxy)methyl)oxazolo[4,5-b]pyridin-2(3H)-one (222-5)

To a solution of 6-bromo-3-((2-(trimethylsilyl)ethoxy)methyl)oxazolo[4,5-b]pyridin-2(3H)-one (100 mg, 0.29 mmol) in DMF (10 ml), Bis(triphenylphosphine)palladium(II) chloride (41 mg, 0.06 mmol) and tetramethyltin (104 mg, 0.6 mmol) were added at r.t. under N₂. After addition, the mixture was stirred at 160° C. with microwave for 30 min. LC-MS showed the starting material was mostly converted to the product. Then added water (10 mL) and extracted with EtOAc (10 mL×3). The organics were then combined and dried (Na₂SO₄) before concentration to dryness. The crude was purified with flash (PE/EtOAc=5:1) to give the product 6-methyl-3-((2-(trimethylsilyl)ethoxy)methyl)oxazolo[4,5-b]pyridin-2(3H)-one (60 mg, 73.9%) as a yellow oil. MS (ESI) m/z 281 [M+H]⁺.

### Step 3. 6-(bromomethyl)-3-((2-(trimethylsilyl)ethoxy)methyl)oxazolo[4,5-b]pyridin-2(3H)-one (222-1)

To a solution of 6-methyl-3-((2-(trimethylsilyl)ethoxy)methyl)oxazolo[4,5-b]pyridin-2(3H)-one (500 mg, 1.78 mmol) and NBS (349 mg, 1.96 mmol) in CCl₄ (5.0 mL), was added AIBN (29 mg, 0.18 mmol) at r.t. under N₂. After addition, the mixture was stirred at 90° C. with microwave for 1 h. LC-MS showed the starting material was mostly converted to the product. Then added water (10 mL) and extracted with EtOAc (10 mL×3). The organics were then combined and dried (Na₂SO₄) before concentration to dryness. The crude was purified by flash (PE/EtOAc=10:1) to give 6-(bromomethyl)-3-((2-(trimethylsilyl)ethoxy)methyl)oxazolo[4,5-b]pyridin-2(3H)-one (160 mg, 25%) as a yellow oil. MS (ESI) m/z 359 [M+H]⁺.

### Synthesis of Compound 2 to 537:

### 2-(4-hydroxybenzyl)-3,4-dihydroisoquinolin-1(2H)-one (2)

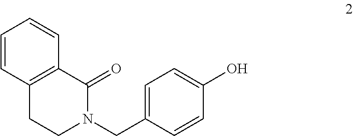 [SMILES: O=C1c2ccccc2CCN1Cc1ccc(O)cc1]

The title compound 2 was prepared according to general procedure A as a white solid (6 mg, 2.2%). MS (ESI) m/z 254 [M+H]⁺. ¹H NMR (400 MHz, DMSO-d₆) δ 9.34 (s, 1H), 7.91 (d, J=7.6 Hz, 1H), 7.51-7.42 (m, 1H), 7.35 (t, J=7.5 Hz, 1H), 7.27 (d, J=7.5 Hz, 1H), 7.12 (d, J=8.2 Hz, 2H), 6.72 (d, J=8.3 Hz, 2H), 4.58 (s, 2H), 3.43 (t, J=6.6 Hz, 2H), 2.92 (t, J=6.6 Hz, 2H).

### 1-(4-hydroxybenzyl)-3,4-dihydroquinolin-2(1H)-one (3)

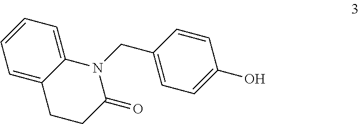 [SMILES: O=C1CCc2ccccc2N1Cc1ccc(O)cc1]

The title compound 3 was prepared according to general procedure A as a white solid (39 mg, 18%). MS (ESI) m/z 254 [M+H]⁺. ¹H NMR (400 MHz, DMSO-d₆) δ 9.27 (s, 1H), 7.20 (d, J=7.2 Hz, 1H), 7.11 (t, J=7.6 Hz, 1H), 7.02 (d, J=8.4 Hz, 2H), 6.94 (d, J=8.0 Hz, 2H), 6.67 (d, J=8.4 Hz, 2H), 5.01 (s, 2H), 2.91 (t, J=7.2 Hz, 2H), 2.66 (t, J=7.2 Hz, 2H).

### 1-(4-hydroxybenzyl)quinolin-2(1H)-one (5)

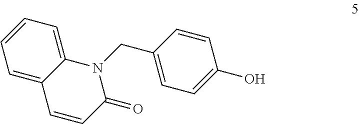 [SMILES: O=c1ccc2ccccc2n1Cc1ccc(O)cc1]

The title compound 5 was prepared according to general procedure A as a white solid (60 mg, 54%). MS (ESI) m/z 252 [M+H]⁺. ¹H NMR (400 MHz, DMSO-d₆) δ 9.34 (s, 1H), 7.96 (d, J=9.2 Hz, 1H), 7.72 (d, J=8.0 Hz, 1H), 7.51 (t, J=7.6 Hz, 1H), 7.43 (d, J=8.8 Hz, 11H), 7.22 (t, J=7.6 Hz, 1H), 7.05 (d, J=8.4 Hz, 2H), 6.70 (t, J=8.4 Hz, 3H), 5.39 (s, 2H).

### 4-(4-hydroxybenzyl)-2H-benzo[b][1,4]oxazin-3(4H)-one (6)

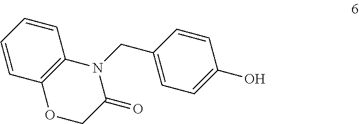 [SMILES: O=C1COc2ccccc2N1Cc1ccc(O)cc1]

The title compound 6 was prepared according to general procedure A as a white solid (93 mg, 42%). MS (ESI) m/z 256 [M+H]⁺. ¹H NMR (400 MHz, DMSO-d₆) δ 9.33 (s, 1H), 7.09 (d, J=8.4 Hz, 2H), 7.07-7.04 (m, 1H), 6.98-6.94 (m, 3H), 6.70 (d, J=8.4 Hz, 2H), 5.03 (s, 2H), 4.75 (s, 2H).

### 6-((1-oxo-3,4-dihydroisoquinolin-2(1H)-yl)methyl)benzo[d]oxazol-2(3H)-one (8)

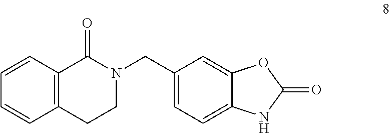 [SMILES: O=C1c2ccccc2CCN1Cc1ccc2[nH]c(=O)oc2c1]

The title compound 8 was prepared according to general procedure B as a white solid (20 mg, 46%). MS (ESI) m/z 295 [M+H]⁺. ¹H NMR (400 MHz, DMSO-d₆) δ 11.60 (s, 1H), 7.92 (dd, J=7.6, 1.6 Hz, 1H), 7.52-7.43 (m, 1H), 7.41-7.33 (m, 1H), 7.33-7.22 (m, 2H), 7.13 (dd, J=8.0, 1.6 Hz, 1H), 7.05 (d, J=8.0 Hz, 1H), 4.70 (s, 2H), 3.49 (t, J=6.6 Hz, 2H), 2.94 (t, J=6.6 Hz, 2H).

### 6-((1,1-dioxidobenzo[d]isothiazol-2(3H)-yl)methyl)benzo[d]oxazol-2(3H)-one (9)

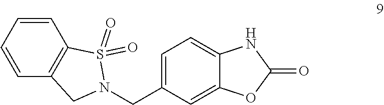 [SMILES: O=c1[nH]c2ccc(CN3Cc4ccccc4S3(=O)=O)cc2o1]

The title compound 9 was prepared according to general procedure B as a white solid (48 mg, 59%). MS (ESI) m/z 317 [M+H]⁺. ¹H NMR (400 MHz, DMSO-d₆) δ 11.68 (s, 1H), 7.90 (d, J=7.6 Hz, 1H), 7.75-7.66 (m, 1H), 7.63-7.59 (m, 1H), 7.57-7.52 (m, 1H), 7.34 (d, J=1.5 Hz, 1H), 7.22 (dd, J=8.0, 1.6 Hz, 1H), 7.10 (d, J=7.9 Hz, 1H), 4.41 (s, 2H), 4.32 (s, 2H).

### 2-(4-hydroxybenzyl)-1,4-dihydroisoquinolin-3(2H)-one (10)

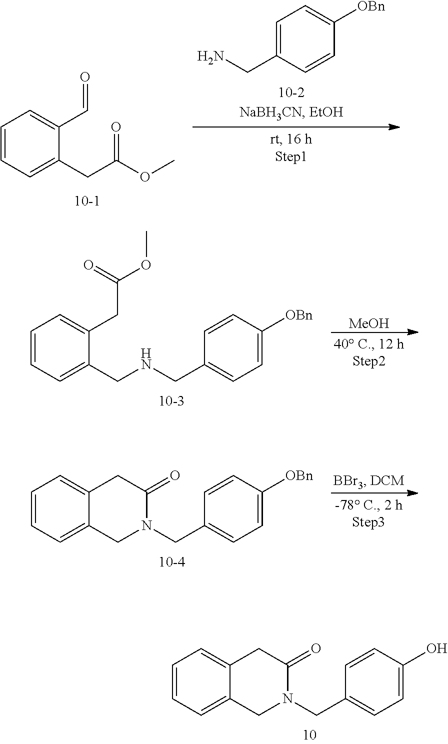

### Step 1: methyl 2-(2-(((4-(benzyloxy)benzyl)amino)methyl)phenyl)acetate (10-3)

Add (4-(benzyloxy)phenyl)methanamine (772 mg, 3.62 mmol)under a N₂ to a solution of methyl 2-(2-formylphenyl)acetate (500 mg, 2.8 mmol) in dry ethanol (10 mL). The resulting mixture was stirred for 16 h at r.t. and cool the mixture to 0° C. Add NaBH₃CN (354 mg, 5.62 mmol) portionwise until the disappearance of the intermediate imine. The reaction mixture was poured into ice water (50 mL) and extracted with EA (50 mL×3), the combined organic phase was washed with brine (100 mL), dried over Na₂SO₄, filtered, and concentrated in vacuo to afford the crude product (400 mg, 38%) as a yellow oil. Mass (m/z): 376 [M+H]⁺.

### Step 2: 2-(4-(benzyloxy)benzyl)-1,4-dihydroisoquinolin-3(2H)-one (10-4)

A mixture of methyl 2-(2-(((4-(benzyloxy)benzyl)amino)methyl)phenyl)acetate (380 mg, 1.01 mmol) in MeOH (10 mL). The reaction mixture was heated to 40° C. for 12 h and LC-MS showed the reaction was completed. The solvent was removed in vacuo and purified by column chromatography on silica gel (MeOH in DCM=0% to 10%) to afford 2-(4-(benzyloxy)benzyl)-1,4-dihydroisoquinolin-3(2H)-one (140 mg, 40%) as a yellow oil. Mass(m/z): 344 [M+H]⁺.

### Step 3: 2-(4-hydroxybenzyl)-1,4-dihydroisoquinolin-3(2H)-one (10)

To a solution of 2-(4-(benzyloxy)benzyl)-1,4-dihydroisoquinolin-3(2H)-one (120 mg, 0.35 mmol) in DCM (10 mL) were added BBr₃ (95 mg, 0.38 mmol) at −78° C. The mixture was stirred for 2 h at −78° C. under N₂. The organic layer was separated and concentrated under vacuum and purified by prep-HPLC to give 2-(4-hydroxybenzyl)-1,4-dihydroisoquinolin-3(2H)-one (28 mg, 32%) as a white solid. MS (ESI) m/z: 254 [M+H]⁺. ¹H NMR (400 MHz, DMSO-d₆) δ 9.34 (s, 1H), 7.32 (m, 4H), 7.05 (d, J=8.4 Hz, 2H), 6.71 (d, J=8.8 Hz, 2H), 4.52 (s, 2H), 4.38 (s, 2H), 3.60 (s, 2H).

### 2-(4-hydroxybenzyl)isoindoline-1,3-dione (11)

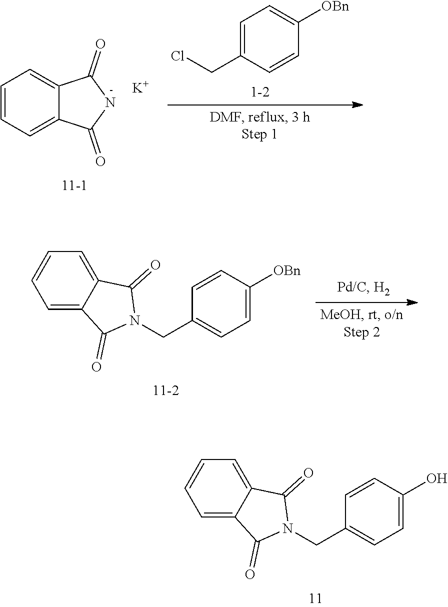 [SMILES: O=C1NC(=O)c2ccccc21 ; [K+] ; ClCc1ccc(OCc2ccccc2)cc1 ; O=C1c2ccccc2C(=O)N1Cc1ccc(OCc2ccccc2)cc1 ; O=C1c2ccccc2C(=O)N1Cc1ccc(O)cc1]

### Step 1. 2-(4-(benzyloxy)benzyl)isoindoline-1,3-dione (11-2)

To a mixture of potassium 1,3-dioxoisoindolin-2-ide (1 g, 4.3 mmol) in DMF (10 mL) was added 1-(benzyloxy)-4-(chloromethyl)benzene (836 mg, 4.51 mmol). The resulting mixture was stirred at reflux for 3 h. The reaction mixture was cooled to ambient temperature. The insoluble solid was collected by filtration and washed with water (100 ml). The collected solid was dried in an oven to afford the title compound (1.39 g, 94.2%) as a white solid. MS (ESI) m/z 344 [M+H]⁺.

### Step 2. 2-(4-hydroxybenzyl)isoindoline-1,3-dione (11)

Pd/C (50 mg, 0.15 mmol) was added to a stirred mixture of 2-(4-(benzyloxy) benzyl)isoindoline-1,3-dione (500 mg, 1.46 mmol) in methanol (10 mL). The mixture was evacuated and backfilled with hydrogen for 3 times. The resulting mixture was stirred at r.t. for overnight under hydrogen atmosphere. The reaction mixture was filtered off and washed with methanol (20 mL). The collected filtrate was concentrated under vacuum. The residue was purified by Prep-HPLC to afford 2-(4-hydroxybenzyl)isoindoline-1,3-dione (168 mg, 45.6%) as a white solid. MS (ESI) m/z 254 [M+H]⁺. ¹H NMR (400 MHz, DMSO-d₆) δ 9.38 (s, 1H), 7.93-7.80 (m, 4H), 7.12 (d, J=8.4 Hz, 2H), 6.70 (d, J=8.4 Hz, 2H), 4.64 (s, 2H).

### 2-(4-hydroxybenzyl)isoindolin-1-one (12)

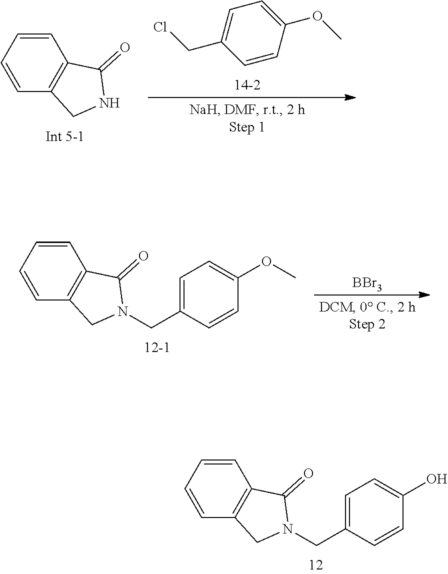

### Step 1. 2-(4-methoxybenzyl)isoindolin-1-one (12-1)

To a stirred solution of isoindolin-1-one (200 mg, 1.5 mmol) in THF (10 mL) was added NaH (60%, 72 mg, 1.8 mmol) at 0° C. for 10 min. Then 1-(chloromethyl)-4-methoxybenzene (283 mg, 1.8 mmol) was added. The resulting mixture was stirred at r.t. for 2 h. The reaction mixture was quenched with water, extracted with EA (3×50 mL). The combined organic phase was washed with brine, dried over anhydrous Na₂SO₄ and concentrated to afford the title compound (379 mg, crude) as a light yellow solid. MS (ESI) m/z 254 [M+H]⁺.

### Step 2. 2-(4-hydroxybenzyl)isoindolin-1-one (12)

To a solution of 2-(4-methoxybenzyl)isoindolin-1-one (379 mg, 1.5 mmol) in DCM (5 mL) was added BBr₃ (1.13 g, 4.5 mmol) at 0° C., and stirred for 2h, and then quenched with water, extracted with DCM (3×50 mL). The combined organic phase was washed with brine, dried over anhydrous Na₂SO₄ and concentrated. The residue was purified by prep-HPLC to afford 2-(4-hydroxybenzyl)isoindolin-1-one (86 mg, 24%) as a white solid. MS (ESI) m/z 240 [M+H]⁺. ¹H NMR (400 MHz, DMSO-d₆) δ 9.35 (s, 1H), 7.70 (d, J=7.5 Hz, 1H), 7.60-7.52 (m, 2H), 7.48 (t, J=7.2 Hz, 1H), 7.09 (d, J=8.0 Hz, 2H), 6.72 (d, J=8.4 Hz, 2H), 4.60 (s, 2H), 4.31 (s, 2H).

### 1-(4-hydroxybenzyl)-1,2-dihydro-3H-indazol-3-one (13)

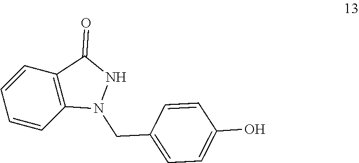 [SMILES: O=c1[nH]n(Cc2ccc(O)cc2)c2ccccc12]

The title compound 13 was prepared according to the procedure described for compound 12 as a white solid (32 mg, 13.3%). MS (ESI) m/z 241 [M+H]⁺. ¹H NMR (400 MHz, DMSO-d₆) δ 10.63 (s, 1H), 9.31 (s, 1H), 7.59 (d, J=8.0 Hz, 1H), 7.50 (d, J=8.5 Hz, 1H), 7.30 (t, J=7.7 Hz, 1H), 7.04 (d, J=8.4 Hz, 2H), 6.97 (t, J=7.4 Hz, 11H), 6.66 (d, J=8.4 Hz, 2H), 5.20 (s, 2H).

### 2-(4-hydroxybenzyl)-5-(phenylamino)isoindoline-1,3-dione (15)

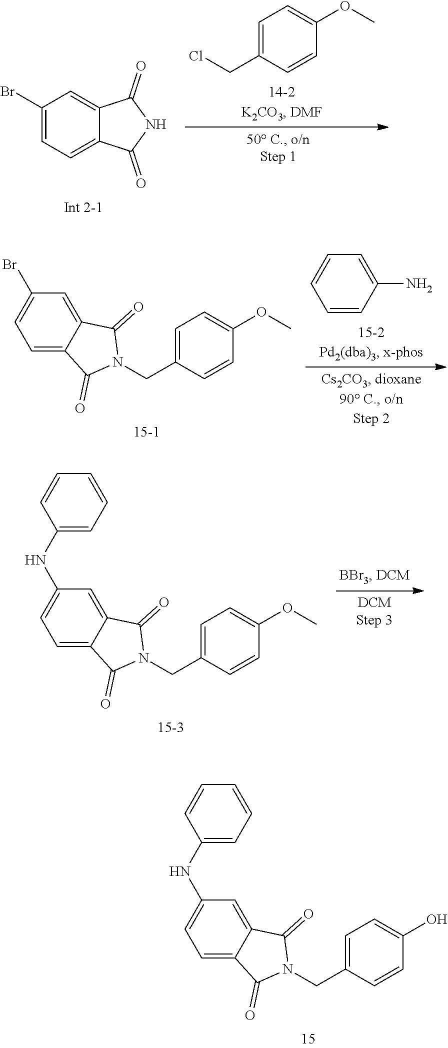

### Step 1. 5-bromo-2-(4-methoxybenzyl)isoindoline-1,3-dione (15-1)

To a stirred solution of 5-bromoisoindoline-1,3-dione (300 mg, 1.33 mmol) in DMF (8 mL) was added 1-(chloromethyl)-4-methoxybenzene (230 mg, 1.46 mmol), K₂CO₃ (367 mg, 2.66 mmol), and stirred at 50° C. overnight. The reaction mixture was quenched with water, and extracted with EA (3×50 mL). The combined organic phase was washed with brine, dried over Na₂SO₄ and concentrated to afford the title compound (450 mg, crude) as a light yellow solid. MS (ESI) m/z 346/348 [M+H]⁺.

### Step 2. 2-(4-methoxybenzyl)-5-(phenylamino)isoindoline-1,3-dione (15-3)

To a solution of 5-bromo-2-(4-methoxybenzyl)isoindoline-1,3-dione (100 mg, 0.29 mmol) in dioxane (5 mL) were added aniline (27 mg, 0.29 mmol), Pd₂(dba)₃ (13 mg, 0.015 mmol), X-Phos (14 mg, 0.03 mmol) and Cs₂CO₃ (190 mg, 0.58 mmol) at r.t. under N₂. The resulting mixture was stirred for 12 h at 90° C. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=1/3) to afford the title compound (65 mg, 63% yield) as a yellow oil. MS (ESI) m/z 359 [M+H]⁺.

### Step 3. 2-(4-hydroxybenzyl)-5-(phenylamino)isoindoline-1,3-dione (15)

To a solution of 2-(4-methoxybenzyl)-5-(phenylamino)isoindoline-1,3-dione (65 mg, 0.18 mmol) in DCM (5 mL) was added BBr₃ (136 mg, 0.54 mmol) at 0° C., and stirred for 2h, and then quenched with water, extracted with DCM (3×50 mL). The combined organic phase was washed with brine, dried over anhydrous Na₂SO₄ and concentrated. The residue was purified by Prep-HPLC to afford 2-(4-hydroxybenzyl)-5-(phenylamino)isoindoline-1,3-dione (23 mg, 37%) as a white solid. MS (ESI) m/z 345 [M+H]⁺. ¹H NMR (400 MHz, DMSO-d₆) δ 9.36 (s, 1H), 9.14 (s, 1H), 7.66 (d, J=8.3 Hz, 1H), 7.38 (t, J=7.7 Hz, 2H), 7.32-7.18 (m, 4H), 7.12-7.04 (m, 3H), 6.66 (d, J=8.4 Hz, 2H), 4.58 (s, 2H).

### 5-bromo-2-(4-hydroxybenzyl)isoindoline-1,3-dione(16)

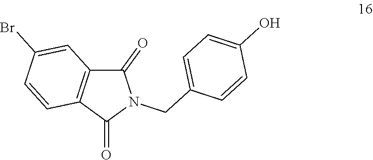 [SMILES: O=C1c2ccc(Br)cc2C(=O)N1Cc1ccc(O)cc1]

The title compound 16 was prepared according to general procedure D as a white solid (26 mg, 34%). MS (ESI) m/z 332 [M+H]⁺. ¹H NMR (400 MHz, DMSO-d₆) δ 9.38 (s, 1H), 8.12-7.98 (m, 2H), 7.81 (d, J=7.9 Hz, 1H), 7.12 (d, J=8.4 Hz, 2H), 6.69 (d, J=8.4 Hz, 2H), 4.63 (s, 2H).

### 2-(4-hydroxybenzyl)-4-(phenylamino)isoindoline-1,3-dione (17)

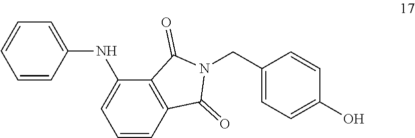 [SMILES: O=C1c2cccc(Nc3ccccc3)c2C(=O)N1Cc1ccc(O)cc1]

The title compound 17 was prepared according to the procedure described for compound 15 as a white solid (27 mg, 31%). MS (ESI) m/z 345 [M+H]⁺. ¹H NMR (400 MHz, DMSO-d₆) δ 9.37 (s, 1H), 8.41 (s, 1H), 7.58 (t, J=7.8 Hz, 1H), 7.43-7.37 (m, 3H), 7.32 (d, J=7.9 Hz, 2H), 7.21 (d, J=7.1 Hz, 1H), 7.18-7.09 (m, 3H), 6.71 (d, J=8.4 Hz, 2H), 4.62 (s, 2H).

### 5-(tert-butyl)-2-(4-hydroxybenzyl)isoindoline-1,3-dione(18)

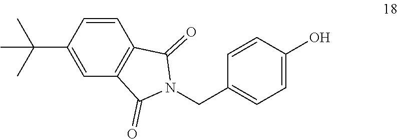 [SMILES: CC(C)(C)c1ccc2c(c1)C(=O)N(Cc1ccc(O)cc1)C2=O]

The title compound 18 was prepared according to general procedure D as a white solid (58 mg, 35%). MS (ESI) m/z 310 [M+H]⁺. ¹H NMR (400 MHz, DMSO-d₆) δ 9.39 (s, 1H), 7.90-7.78 (m, 3H), 7.11 (d, J=8.4 Hz, 2H), 6.69 (d, J=7.6 Hz, 2H), 4.63 (s, 2H), 1.34 (s, 9H).

### 6-bromo-2-(4-hydroxybenzyl) isoindolin-1-one (19)

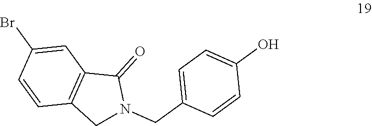 [SMILES: O=C1c2cc(Br)ccc2CN1Cc1ccc(O)cc1]

The title compound 19 was prepared according to general procedure A as a yellow solid (7 mg, 24%). MS (ESI) m/z 318 [M+H]⁺. ¹H NMR (400 MHz, CD₃OD) δ 7.91 (s, 1H), 7.72 (d, J=8.1 Hz, 11H), 7.44 (dd, J=8.0, 2.4 Hz, 11H), 7.15 (dd, J=8.4, 2.5 Hz, 2H), 6.76 (dd, J=8.5, 2.7 Hz, 2H), 4.69 (s, 2H), 4.31 (s, 2H).

### 5-bromo-2-(4-hydroxybenzyl) isoindolin-1-one (20)

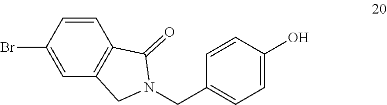 [SMILES: O=C1c2ccc(Br)cc2CN1Cc1ccc(O)cc1]

The title compound 20 was prepared according to general procedure A as a white solid (8 mg, 17%). MS (ESI) m/z 318 [M+H]⁺. ¹H NMR (400 MHz, DMSO-d₆) δ 9.40 (s, 1H), 7.81 (s, 1H), 7.69-7.63 (m, 2H), 7.09 (d, J=8.3 Hz, 2H), 6.73 (d, J=8.3 Hz, 2H), 4.59 (s, 2H), 4.31 (s, 2H).

### 4-bromo-2-(4-hydroxybenzyl) isoindolin-1-one (21)

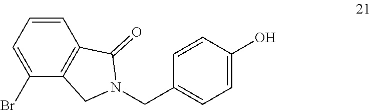 [SMILES: O=C1c2cccc(Br)c2CN1Cc1ccc(O)cc1]

The title compound 21 was prepared according to general procedure A as a white solid (3 mg, 26%). MS (ESI) m/z 318 [M+H]⁺. ¹H NMR (400 MHz, CD₃OD) δ 7.79 (d, J=7.6 Hz, 1H), 7.74 (d, J=8.0 Hz, 1H), 7.45 (t, J=7.7 Hz, 1H), 7.17 (d, J=8.3 Hz, 2H), 6.78 (d, J=8.3 Hz, 2H), 4.72 (s, 2H), 4.26 (s, 2H).

### 2-(4-hydroxybenzyl)-5-phenoxyisoindoline-1,3-dione (22)

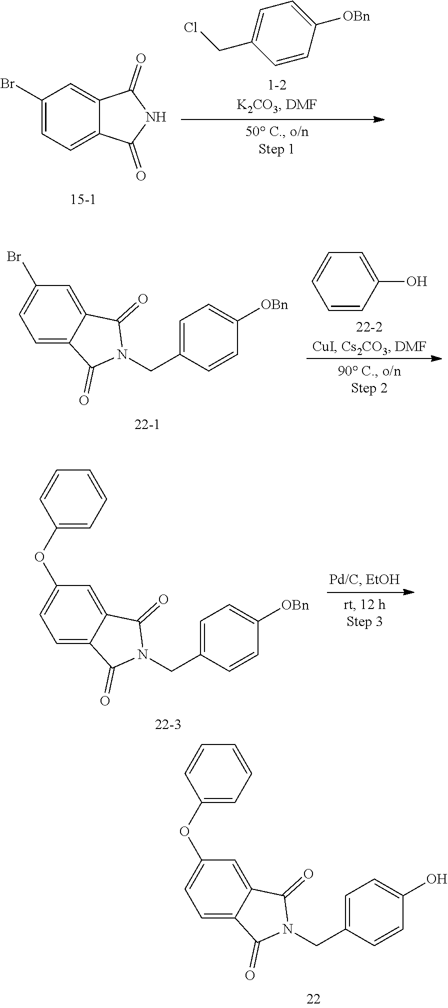

### Step 1. 2-(4-(benzyloxy)benzyl)-5-bromoisoindoline-1,3-dione (22-1)

To a stirred solution of 5-bromoisoindoline-1,3-dione (1.5 g, 6.64 mmol) in DMF (15 mL) was added 1-(benzyloxy)-4-(chloromethyl)benzene (1.55 g, 6.64 mmol), K₂CO₃ (1.83 g, 13.3 mmol), and stirred at 50° C. overnight. The reaction mixture was quenched with water, and extracted with EA (3×50 mL). The combined organic phase was washed with brine, dried over anhydrous N₂SO₄ and concentrated to afford the title compound (2.7 g, crude) as a light yellow solid. MS (ESI) m/z 422/424 [M+H]⁺.

### Step 2. 2-(4-(benzyloxy)benzyl)-5-phenoxyisoindoline-1,3-dione (22-3)

To a solution of 2-(4-(benzyloxy)benzyl)-5-bromoisoindoline-1,3-dione (100 mg, 0.24 mmol) in DMF (3 mL) were added CuI (2 mg, 0.012 mmol), Cs₂CO₃ (155 mg, 0.48 mmol) at r.t. under N₂. The resulting mixture was stirred for 15 h at 100′C. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=1/3) to afford the title compound (20 mg, 19.3%) as a yellow oil. MS (ESI) m/z 436.1 [M+H]⁺.

### Step 3. 2-(4-hydroxybenzyl)-5-phenoxyisoindoline-1,3-dione (22)

To a solution of 2-(4-(benzyloxy)benzyl)-5-phenoxyisoindoline-1,3-dione (20 mg, 0.046 mmol) in EtOH (5 mL) was added Pd/C (5 mg). The mixture was evacuated and backfilled three times with hydrogen. The resulting mixture was stirred at r.t. overnight. The reaction mixture was filtered off and the filtrate was concentrated. The residue was purified by Prep-HPLC to afford the title compound as a white solid (1 mg, 6.3%). MS (ESI) m/z 346 [M+H]⁺. ¹H NMR (400 MHz, DMSO-d₆) δ 9.38 (s, 1H), 7.88 (d, J=8.2 Hz, 1H), 7.50 (t, J=7.8 Hz, 2H), 7.39-7.25 (m, 3H), 7.18 (d, J=8.0 Hz, 2H), 7.10 (d, J=8.3 Hz, 2H), 6.69 (d, J=8.3 Hz, 2H), 4.62 (s, 2H).

### 2-(4-hydroxybenzyl)-1-methyl-1,2-dihydro-3H-indazol-3-one (23)

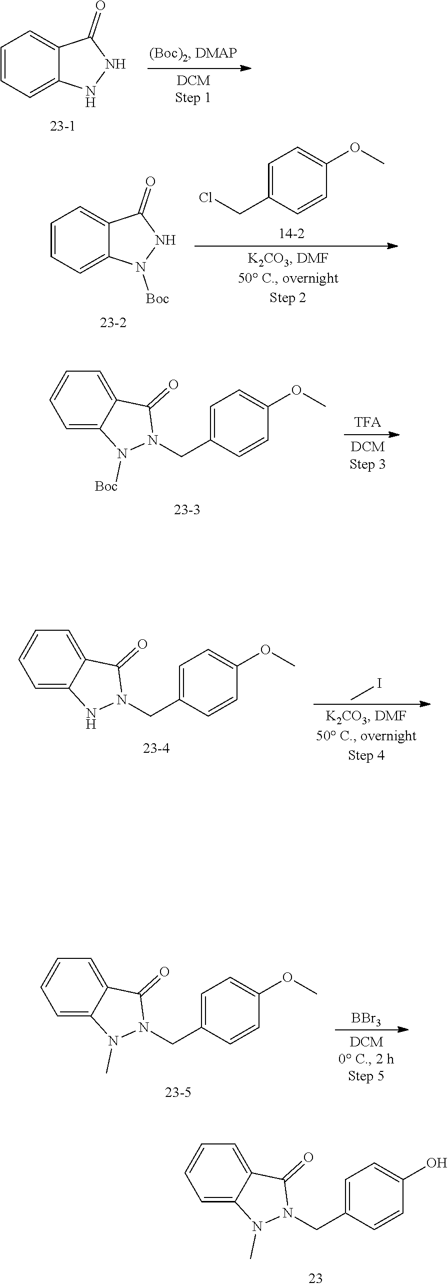

### Step 1. tert-butyl 3-oxo-2,3-dihydro-1H-indazole-1-carboxylate (23-2)

To a stirred solution of 1,2-dihydro-3H-indazol-3-one (500 mg, 3.73 mmol) in DCM (20 mL) was added (Boc)₂O (813 mg, 3.73 mmol), DMAP (45 mg, 0.37 mmol), and stirred at rt overnight. The reaction mixture was quenched with water, and extracted with DCM (3×50 mL). The organic layer was separated, dried over anhydrous Na₂SO₄ and concentrated under vacuum. The residue was purified by silica gel flash column chromatography (PE/EA=1/3) to afford the title compound (800 mg, 91.7%) as a yellow solid. MS (ESI) m/z 235 [M+H]⁺.

### Step 2. tert-butyl 2-(4-methoxybenzyl)-3-oxo-2,3-dihydro-1H-indazole-1-carboxylate (23-3)

To a stirred solution of tert-butyl 3-oxo-2,3-dihydro-1H-indazole-1-carboxylate (300 mg, 1.28 mmol) in DMF (10 mL) was added 1-(chloromethyl)-4-methoxybenzene (201 mg, 1.28 mmol), K₂CO₃ (354 mg, 2.56 mmol), and stirred at 50° C. overnight. The reaction mixture was quenched with water, extracted with EA (3×50 mL). The combined organic phase was washed with brine, dried over anhydrous Na₂SO₄ and concentrated to afford the title compound (450 mg, 99.3%) as a yellow oil. MS (ESI) m/z 355 [M+H]⁺.

### Step 3. 2-(4-methoxybenzyl)-1,2-dihydro-3H-indazol-3-one (23-4)

To a stirred solution of tert-butyl 2-(4-methoxybenzyl)-3-oxo-2,3-dihydro-1H-indazole-1-carboxylate (450 mg, 1.27 mmol) in DCM (10 mL) was added TFA (5 mL), and stirred at rt for 30 min. The reaction mixture was concentrated and quenched with NaHCO₃(aq.), extracted with DCM (3×50 mL). The combined organic phase was washed with brine, dried over anhydrous Na₂SO₄ and concentrated to afford the title compound (320 mg, crude) as a yellow oil. MS (ESI) m/z 255 [M+H]⁺.

### Step 4. 2-(4-methoxybenzyl)-1-methyl-1,2-dihydro-3H-indazol-3-one (23-5)

To a stirred solution of 2-(4-methoxybenzyl)-1,2-dihydro-3H-indazol-3-one (66 mg, 0.26 mmol) in DMF (5 mL) was added iodomethane (37 mg, 0.26 mmol), K₂CO₃ (72 mg, 0.52 mmol), and stirred at 50° C. overnight. The reaction mixture was quenched with water, extracted with EA (3×50 mL). The combined organic phase was washed with brine, dried over anhydrous Na₂SO₄ and concentrated to afford the title compound (50 mg, 71.8%) as yellow oil. MS (ESI) m/z 269 [M+H]⁺.

### Step 5. 2-(4-hydroxybenzyl)-1-methyl-1,2-dihydro-3H-indazol-3-one (23)

To a solution of 2-(4-methoxybenzyl)-1-methyl-1,2-dihydro-3H-indazol-3-one (50 mg, 0.19 mmol) in DCM (5 mL) was added BBr₃ (140 mg, 0.56 mmol) at 0° C., and reacted for 2h, and then quenched with water, extracted with DCM (3×50 mL). The combined organic phase was washed with brine, dried over anhydrous Na₂SO₄ and concentrated. The residue was purified by Prep-HPLC to afford 2-(4-hydroxybenzyl)-1-methyl-1,2-dihydro-3H-indazol-3-one as a white solid (9 mg, 19%). MS (ESI) m/z 255 [M+H]⁺. ¹H NMR (400 MHz, DMSO-d₆) δ 9.36 (s, 1H), 7.69 (d, J=7.8 Hz, 1H), 7.57 (t, J=7.7 Hz, 11H), 7.44 (d, J=8.3 Hz, 1H), 7.16 (t, J=7.4 Hz, 1H), 7.06 (d, J=8.1 Hz, 2H), 6.67 (d, J=8.1 Hz, 2H), 4.94 (s, 2H), 3.24 (s, 3H).

### 1-ethyl-2-(4-hydroxybenzyl)-1,2-dihydro-3H-indazol-3-one (24)

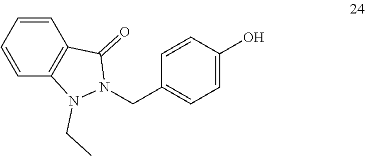 [SMILES: CCn1c2ccccc2c(=O)n1Cc1ccc(O)cc1]

The title compound 24 was prepared according to the procedure described for compound 23 as a white solid (10 mg, 20%). MS (EST) m/z 269 [M+H]⁺. 11H NMR (400 MHz, DMSO-d₆) δ 9.35 (s, 11H), 7.69 (d, J=7.8 Hz, 11H), 7.56 (t, J=7.7 Hz, 11H), 7.43 (d, J=8.3 Hz, 11H), 7.14 (t, J=7.4 Hz, 1H), 7.07 (d, J=8.2 Hz, 2H), 6.66 (d, J=8.2 Hz, 2H), 4.91 (s, 2H), 3.90 (q, J=6.9 Hz, 2H), 0.67 (t, J=6.9 Hz, 3H).

### 2-(4-hydroxybenzyl)-1-propyl-1,2-dihydro-3H-indazol-3-one (25)

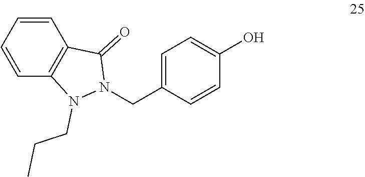 [SMILES: CCCn1c2ccccc2c(=O)n1Cc1ccc(O)cc1]

The title compound 25 was prepared according to the procedure described for compound 23 as a white solid (10 mg, 19.8%). MS (ESI) m/z 283 [M+H]⁺. 1H NMR (400 MHz, DMSO-d₆) δ 9.35 (s, 1H), 7.68 (d, J=7.8 Hz, 11H), 7.54 (t, J=8.0 Hz, 1H), 7.44 (d, J=8.3 Hz, 11H), 7.11 (t, J 7.4 Hz, 11H), 7.07 (d, J=8.0 Hz, 2H), 6.66 (d, J=8.4 Hz, 2H), 4.92 (s, 2H), 3.80 (t, J=7.6 Hz, 2H), 1.19-1.06 (m, 2H), 0.67 (t, J=7.4 Hz, 3H).

### 2-(4-hydroxybenzyl)-5-isopropylisoindoline-1,3-dione (26)

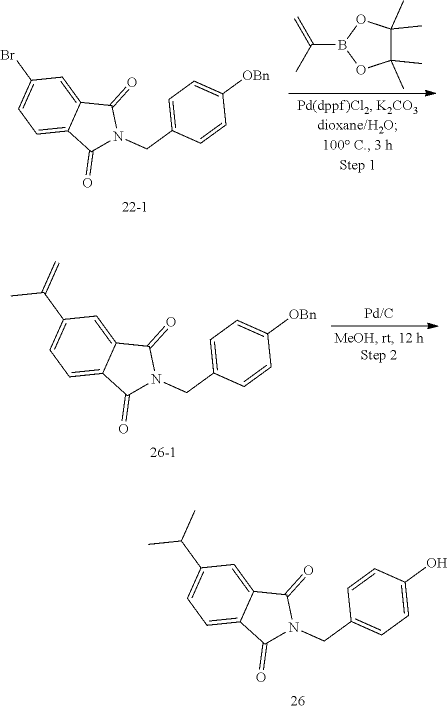 [SMILES: O=C1c2ccc(Br)cc2C(=O)N1Cc1ccc(OCc2ccccc2)cc1 ; C=C(C)B1OC(C)(C)C(C)(C)O1 ; C=C(C)c1ccc2c(c1)C(=O)N(Cc1ccc(OCc3ccccc3)cc1)C2=O ; CC(C)c1ccc2c(c1)C(=O)N(Cc1ccc(O)cc1)C2=O]

### Step 1. 2-(4-(benzyloxy)benzyl)-5-(prop-1-en-2-yl)isoindoline-1,3-dione (26-1)

To a solution of 2-(4-(benzyloxy)benzyl)-5-bromoisoindoline-1,3-dione (100 mg, 0.24 mmol) in 1-4, dioxane (3 mL) and water (1 mL) were added Pd(dppf)Cl₂ (10 mg, 0.012 mmol), K₂CO₃ (65 mg, 0.47 mmol) at r.t. under N₂. The resulting mixture was stirred for 3 h at 100° C. The mixture was diluted with EA (30 mL) and washed with water (3×20 mL). The organic layer was separated, dried over anhydrous N₂SO₄ and concentrated under vacuum. The residue was purified by silica gel flash column chromatography (PE/EA=1/3) to afford the title compound (60 mg, 65.8%) as a yellow oil. MS (ESI) m/z 384 [M+H]⁺.

### Step 2. 2-(4-hydroxybenzyl)-5-isopropylisoindoline-1,3-dione (26)

To a solution of 2-(4-(benzyloxy)benzyl)-5-(prop-1-en-2-yl)isoindoline-1,3-dione (60 mg, 0.16 mmol) in MeOH (5 mL) was added Pd/C (6 mg). The mixture was evacuated and backfilled three times with hydrogen. The resulting mixture was stirred at r.t. overnight. The reaction mixture was filtered off and the filtrate was concentrated. The residue was purified by Prep-HPLC to afford 2-(4-hydroxybenzyl)-5-isopropylisoindoline-1,3-dione (14 mg, 30.2%) as a white solid. MS (ESI) m/z 296 [M+H]⁺. 1H NMR (400 MHz, DMSO-d₆) δ 9.37 (s, 1H), 7.85-7.62 (m, 3H), 7.11 (d, J=8.3 Hz, 2H), 6.69 (d, J=8.3 Hz, 2H), 4.63 (s, 2H), 3.15-3.05 (m, 1H), 1.25 (d, J=6.9 Hz, 6H).

### 2-(2-(4-hydroxybenzyl)-3-oxoisoindolin-1-yl)acetonitrile (27)

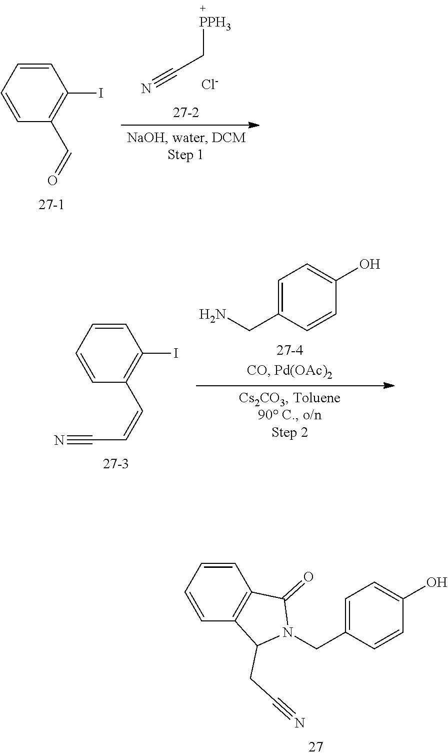

### Step 1. (Z)-3-(2-iodophenyl)acrylonitrile (27-3)

To a solution of 2-iodobenzaldehyde (500 mg, 2.15 mmol) and (cyanomethyl) triphenylphosphonium chloride (728 mg, 2.15 mmol) in DCM (5 mL) was added NaOH (95 mg, 2.37 mmol) in water (2 mL). The mixture was stirred for 1 h, the mixture was diluted with EtOAc, and washed with water. The organic layer was separated, dried over anhydrous Na₂SO₄ and concentrated under vacuum. The residue was purified by prep TLC (PE/EA=3/1) to give (Z)-3-(2-iodophenyl)acrylonitrile (300 mg, 54%) as a yellow oil. MS (ESI) m/z 256 [M+H]⁺.

### Step 2. 2-(2-(4-hydroxybenzyl)-3-oxoisoindolin-1-yl)acetonitrile (27)

To a solution of (Z)-3-(2-iodophenyl)acrylonitrile (100 mg, 0.39 mmol), 4-(aminomethyl)phenol (53 mg, 0.43 mmol), Cs₂CO₃ (256 mg, 0.78 mmol), PPh₃ (103 mg, 0.39 mmol) and Pd(OAc)₂ (18 mg, 0.07 mmol) in Toluene (2 mL) in a 50 mL round bottom flask equipped with a magnetic stir bar and nitrogen inlet. The flask was evacuated and purged with nitrogen three times, and then evacuated and placed under CO (1 atm). The mixture was stirred at 90° C. overnight. The solution was concentrated and purified by prep HPLC to give 2-(2-(4-hydroxybenzyl)-3-oxoisoindolin-1-yl)acetonitrile (4 mg, 3%) as a white solid. MS (ESI) m/z 279 [M+H]⁺. ¹H NMR (400 MHz, DMSO-d₆) δ 9.40 (s, 1H), 7.76 (d, J=7.5 Hz, 1H), 7.70-7.65 (m, 2H), 7.62-7.52 (m, 1H), 7.14 (d, J=8.2 Hz, 2H), 6.71 (d, J=8.3 Hz, 2H), 4.97 (d, J=15.0 Hz, 1H), 4.64 (t, J=4.2 Hz, 1H), 4.30 (d, J=15.0 Hz, 1H), 3.41 (d, J=4.1 Hz, 2H).

### 2-(4-hydroxybenzyl)-3-oxoisoindoline-1-carbonitrile (28)

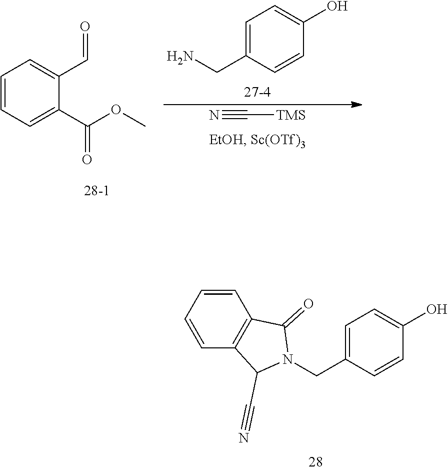

Methyl 2-formylbenzoate (164 mg, l mmol), 4-(aminomethyl)phenol (123 mg, 1 mmol), trimethylsilanecarbonitrile (198 mg, 2 mmol) and Scandium trifluoromethanesulfonate (123 mg, 0.25 mmol) were dissolved in EtOH (2 mL), and the solution was stirred at r.t. for 7 h. After completing the reaction, the mixture was concentrated in vacuo and the crude product was purified by prep HPLC to give 2-(4-hydroxybenzyl)-3-oxoisoindoline-1-carbonitrile (50 mg, 19%) as a white solid. MS (ESI) m/z 265 [M+H]⁺. ¹H NMR (400 MHz, CDCl₃) δ 7.93 (d, J=7.4 Hz, 1H), 7.70-7.55 (m, 3H), 7.24 (d, J=8.0 Hz, 2H), 6.83 (d, J=8.0 Hz, 2H), 5.42 (d, J=14.9 Hz, 1H), 5.08 (s, 1H), 4.25 (d, J=14.9 Hz, 1H).

### 7-bromo-2-(4-hydroxybenzyl)isoindolin-1-one (29)

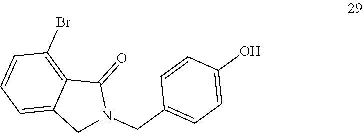 [SMILES: O=C1c2c(Br)cccc2CN1Cc1ccc(O)cc1]

The title compound 29 was prepared according to general procedure A as a yellow solid (6 mg, 9%). MS (ESI) m/z 318 [M+H]⁺. ¹H NMR (400 MHz, CD₃OD) δ 7.62 (d, J=7.6 Hz, 1H), 7.48-7.41 (m, 2H), 7.15 (d, J=8.4 Hz, 2H), 6.76 (d, J=8.4 Hz, 2H), 4.67 (s, 2H), 4.29 (s, 2H).

### 2-(4-hydroxybenzyl)-5-methoxyisoindoline-1,3-dione (30)

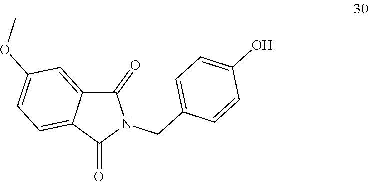 [SMILES: COc1ccc2c(c1)C(=O)N(Cc1ccc(O)cc1)C2=O]

The title compound 30 was prepared according to general procedure D as a white solid (48 mg, 42%). MS (ESI) m/z 284 [M+H]⁺. ¹H NMR (400 MHz, DMSO-d₆) δ 9.37 (s, 1H), 7.80 (d, J=8.3 Hz, 1H), 7.39 (s, 1H), 7.31 (dd, J=8.3, 1.6 Hz, 1H), 7.11 (d, J=8.3 Hz, 2H), 6.69 (d, J=8.3 Hz, 2H), 4.61 (s, 2H), 3.92 (s, 3H).

### 2-(4-hydroxybenzyl)-3-methylisoindolin-1-one (31)

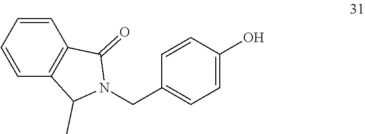 [SMILES: CC1c2ccccc2C(=O)N1Cc1ccc(O)cc1]

The title compound 31 was prepared according to general procedure A as a white solid (30 mg, 39%). MS (ESI) m/z 254 [M+H]⁺. 1H NMR (400 MHz, DMSO-d₆) δ 9.35 (s, 1H), 7.70 (d, J=7.5 Hz, 1H), 7.62-7.56 (m, 2H), 7.53-7.45 (m, 1H), 7.10 (d, J=8.1 Hz, 2H), 6.70 (d, J=8.1 Hz, 2H), 4.94 (d, J=15.0 Hz, 1H), 4.37 (q, J=6.7 Hz, 11H), 4.24 (d, J=15.0 Hz, 11H), 1.37 (d, J=6.7 Hz, 3H).

### 2-(4-hydroxybenzyl)-6-methylisoindolin-1-one (32)

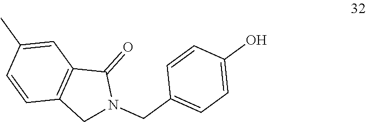 [SMILES: Cc1ccc2c(c1)C(=O)N(Cc1ccc(O)cc1)C2]

The title compound 32 was prepared according to general procedure A as a white solid (32 mg, 42%). MS (ESI) m/z 254 [M+H]⁺. ¹H NMR (400 MHz, DMSO-d₆) δ 9.37 (s, 1H), 7.51 (s, 1H), 7.43-7.37 (m, 2H), 7.08 (d, J=8.0 Hz, 2H), 6.72 (d, J=8.1 Hz, 2H), 4.58 (s, 2H), 4.25 (s, 2H), 2.39 (s, 3H).

### 6-((1-oxoisoindolin-2-yl)methyl)benzo[d]oxazol-2(3H)-one (33)

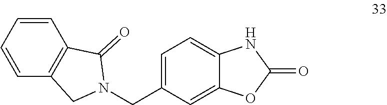 [SMILES: O=C1c2ccccc2CN1Cc1ccc2[nH]c(=O)oc2c1]

The title compound 33 was prepared according to general procedure B as an off-white solid (11 mg, 77%). MS (ESI) m/z 281 [M+H]⁺. ¹H NMR (400 MHz, DMSO-d₆) δ 11.61 (s, 1H), 7.72 (d, J=7.5 Hz, 1H), 7.61-7.54 (m, 2H), 7.50 (t, J=7.3 Hz, 1H), 7.23 (s, 1H), 7.12-7.02 (m, 2H), 4.73 (s, 2H), 4.37 (s, 2H).

### 2-(4-hydroxybenzyl)-4-methylisoindolin-1-one (34)

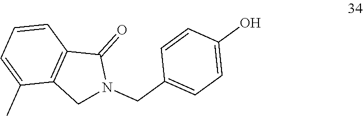 [SMILES: Cc1cccc2c1CN(Cc1ccc(O)cc1)C2=O]

The title compound 34 was prepared according to general procedure A as a white solid (22 mg, 29%). MS (ESI) m/z 254 [M+H]⁺. ¹H NMR (400 MHz, DMSO-d₆) δ 9.37 (s, 1H), 7.52 (dd, J=6.5, 1.9 Hz, 1H), 7.42-7.36 (m, 2H), 7.10 (d, J=8.2 Hz, 2H), 6.73 (d, J=8.2 Hz, 2H), 4.60 (s, 2H), 4.27 (s, 2H), 2.27 (s, 3H).

### 2-(4-hydroxybenzyl)-5-methoxyisoindolin-1-one (35)

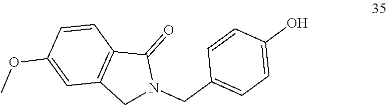 [SMILES: COc1ccc2c(c1)CN(Cc1ccc(O)cc1)C2=O]

The title compound 35 was prepared according to general procedure A as an off-white solid (20 mg, 17%). MS (ESI) m/z 270 [M+H]⁺. ¹H NMR (400 MHz, DMSO-d₆) δ 9.43 (s, 1H), 7.60 (d, J=8.4 Hz, 1H), 7.13-6.99 (m, 4H), 6.72 (d, J=8.2 Hz, 2H), 4.56 (s, 2H), 4.24 (s, 2H), 3.81 (s, 3H).

### 2-(4-hydroxybenzyl)-5-(pyrrolidin-1-yl)isoindoline-1,3-dione (36)

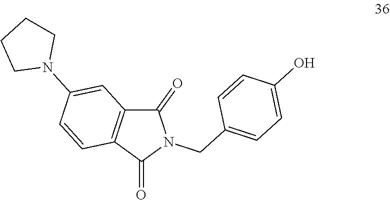 [SMILES: O=C1c2ccc(N3CCCC3)cc2C(=O)N1Cc1ccc(O)cc1]

The title compound 36 was prepared according to the procedure described for compound 15 as a white solid (3 mg, 6.1%). MS (ESI) m/z 323 [M+H]⁺. ¹H NMR (400 MHz, DMSO-d₆) δ 9.34 (s, 11H), 7.60 (d, J=8.4 Hz, 1H), 7.08 (d, J=8.3 Hz, 2H), 6.88 (d, J=2.2 Hz, 11H), 6.77 (dd, J=8.3, 2.3 Hz, 1H), 6.68 (d, J=8.4 Hz, 2H), 4.57 (s, 2H), 3.42-3.36 (m, 4H), 2.03-1.95 (m, 4H).

### 2-((1H-benzo[d][1,2,3]triazol-5-yl)methyl)-5-methylisoindoline-1,3-dione (37)

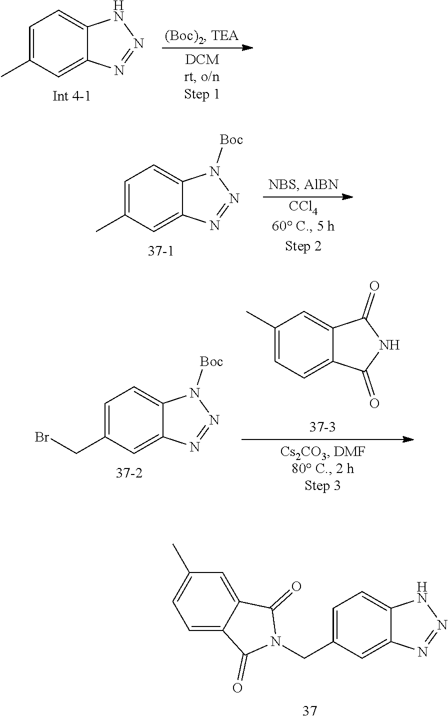

### Step 1. tert-butyl 5-methyl-1H-benzo[d][1,2,3]triazole-1-carboxylate (37-1)

To a stirred solution of 5-methyl-1H-benzo[d][1,2,3]triazole (500 mg, 3.76 mmol) in DCM (20 mL) was added (Boc)₂O (901 mg, 4.14 mmol), TEA (760 mg, 7.52 mmol), and stirred at rt overnight. The reaction mixture was quenched with water, extracted with DCM (3×50 mL). The organic layer was separated, dried over anhydrous Na₂SO₄ and concentrated under vacuum to afford the title compound (800 mg, crude) as a yellow solid. MS (ESI) m/z 234 [M+H]⁺.

### Step 2. tert-butyl 5-(bromomethyl)-1H-benzo[d][1,2,3]triazole-1-carboxylate (37-2)

To a stirred solution of tert-butyl 5-methyl-1H-benzo[d][1,2,3]triazole-1-carboxylate (200 mg, 0.86 mmol) in CCl₄ (10 mL) was added NBS (183 mg, 1.03 mmol), AIBN (20 mg, 0.09 mmol), and reacted at 60° C. for 5 h. The reaction mixture was quenched with water, extracted with EA (3×50 mL). The combined organic phase was washed with brine, dried over anhydrous Na₂SO₄ and concentrated to afford the title compound (65 mg, 24.3%) as a yellow oil. MS (ESI) m/z 312/314 [M+H]⁺.

### Step 3. 2-((1H-benzo[d][1,2,3]triazol-5-yl)methyl)-5-methylisoindoline-1,3-dione (37)

To a solution of tert-butyl 5-(bromomethyl)-1H-benzo[d][1,2,3]triazole-1-carboxylate (65 mg, 0.31 mmol) in DMF (5 mL) was added 5-methylisoindoline-1,3-dione (50 mg, 0.31 mmol) Cs₂CO₃ (202 mg, 0.62 mmol), and stirred at 50° C. overnight. The reaction was then quenched with water, and extracted with EA (3×50 mL). The combined organic phase was washed with brine, dried over anhydrous Na₂SO₄ and concentrated. The residue was purified by Prep-HPLC to afford 2-((1H-benzo[d][1,2,3]triazol-5-yl)methyl)-5-methyl isoindoline-1,3-dione (6 mg, 6.7%) as a white solid. MS (ESI) m/z 293 [M+H]⁺. ¹H NMR (400 MHz, DMSO-d₆) δ 15.55 (s, 1H), 7.87 (d, J=8.6 Hz, 1H), 7.82-7.76 (m, 2H), 7.74 (s, 1H), 7.66 (d, J=7.7 Hz, 1H), 7.37 (d, J=8.6 Hz, 1H), 4.92 (s, 2H), 2.48 (s, 3H).

### 2-(4-hydroxybenzyl)-1,3-dioxoisoindoline-5-carboxamide (38)

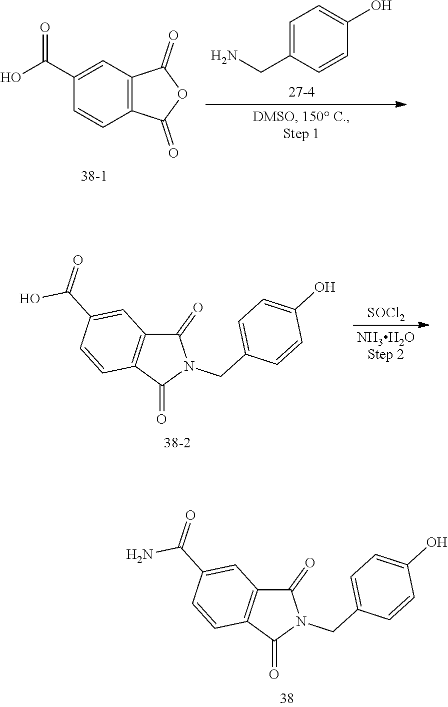

### Step 1. 2-(4-hydroxybenzyl)-1,3-dioxoisoindoline-5-carboxylic acid (38-2)

To a solution of 1,3-dioxo-1,3-dihydroisobenzofuran-5-carboxylic acid (384 mg, 2 mmol) and 4-(aminomethyl)phenol (246 mg, 2 mmol) in DMSO (3 mL). The mixture was irradiated with microwave radiation for 10 min at 150° C., and then the mixture was diluted with water, and extracted with EtOAc. The organic layer was separated, dried over anhydrous Na₂SO₄ and concentrated to give 2-(4-hydroxybenzyl)-1,3-dioxoisoindoline-5-carboxylic acid (400 mg) as an off-white solid, which was used in the next step without further purification. MS (ESI) m/z 298 [M+H]⁺.

### Step 2. 2-(4-hydroxybenzyl)-1,3-dioxoisoindoline-5-carboxamide (38)

A solution of 2-(4-hydroxybenzyl)-1,3-dioxoisoindoline-5-carboxylic acid (200 mg, 0.67 mmol) in SOCl₂ (2.0 mL) was stirred at 80° C. under N₂ for 2 h, the mixture was concentrated under vacuum, the crude was diluted with DCM (2 mL), then added in Ammonium hydroxide (1 mL), and stirred for 2 h, the mixture was diluted with EtOAc, and washed with water. The organic layer was separated, dried over anhydrous Na₂SO₄ and concentrated under vacuum. The residue was purified by prep HPLC to give 2-(4-hydroxybenzyl)-1,3-dioxoisoindoline-5-carboxamide (13 mg, 6%) as a white solid. MS (ESI) m/z 297 [M+H]⁺. 11H NMR (400 MHz, DMSO-d₆) δ 9.39 (s, 1H), 8.32 (s, 2H), 8.30 (d, J=7.7 Hz, 1H), 7.96 (d, J=7.7 Hz, 11H), 7.72 (s, 11H), 7.13 (d, J=8.2 Hz, 2H), 6.70 (d, J=8.2 Hz, 2H), 4.66 (s, 2H).

### 6-(4-hydroxybenzyl)-6,7-dihydro-5H-pyrrolo[3,4-b]pyridin-5-one (39)

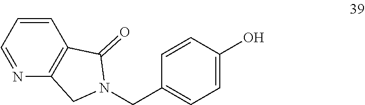 [SMILES: O=C1c2cccnc2CN1Cc1ccc(O)cc1]

The title compound 39 was prepared according to general procedure A as an off-white solid (13 mg, 18.0%). MS (ESI) m/z 241 [M+H]⁺. ¹H NMR (400 MHz, DMSO-d₆) δ 9.37 (s, 1H), 8.74 (d, J=4.9 Hz, 1H), 8.11 (d, J=7.7 Hz, 1H), 7.52 (dd, J=7.5, 5.1 Hz, 1H), 7.12 (d, J=8.2 Hz, 2H), 6.73 (d, J=8.2 Hz, 2H), 4.63 (s, 2H), 4.38 (s, 2H).

### 6-(4-hydroxybenzyl)-5,6-dihydro-7H-pyrrolo[3,4-b]pyridin-7-one (40)

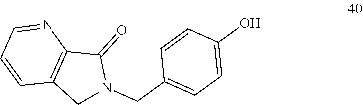 [SMILES: O=C1c2ncccc2CN1Cc1ccc(O)cc1]

The title compound 40 was prepared according to general procedure A as a yellow solid (12 mg, 8.5%). MS (ESI) m/z 241 [M+H]⁺. 1H NMR (400 MHz, CDCl₃) δ 8.79 (d, J=4.6 Hz, 1H), 7.76 (d, J=7.7 Hz, 11H), 7.41 (dd, J=7.5, 4.9 Hz, 11H), 7.19 (d, J=8.2 Hz, 2H), 6.83 (d, J=8.2 Hz, 2H), 4.78 (s, 2H), 4.26 (s, 2H).

### 5-ethyl-2-(4-hydroxybenzyl)isoindoline-1,3-dione (41)

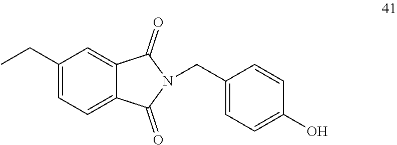 [SMILES: CCc1ccc2c(c1)C(=O)N(Cc1ccc(O)cc1)C2=O]

The title compound 41 was prepared according to the procedure described for compound 26 as a yellow solid (8 mg, 8.2%) as a white solid. MS (ESI) m/z 282 [M+H]⁺. 1′H NMR (400 MHz, DMSO-d₆) δ 9.37 (s, 11H), 7.79 (d, J=7.6 Hz, 1H), 7.74 (s, 1H), 7.68 (d, J=7.6 Hz, 11H), 7.11 (d, J=8.2 Hz, 2H), 6.69 (d, J=8.3 Hz, 2H), 4.62 (s, 2H), 2.78 (q, J=7.6 Hz, 2H), 1.22 (t, J=7.5 Hz, 3H).

### 2-(4-hydroxybenzyl)-1,2-dihydro-3H-pyrrolo[3,4-c]pyridin-3-one (42)

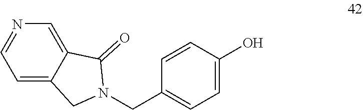 [SMILES: O=C1c2cnccc2CN1Cc1ccc(O)cc1]

The title compound 42 was prepared according to general procedure A as a yellow solid (11 mg, 12.8%). MS (ESI) m/z 241 [M+H]⁺. ¹H NMR (400 MHz, CD₃OD) δ 8.96 (s, 1H), 8.70 (d, J=4.4 Hz, 11H), 7.63 (d, J=4.8 Hz, 1H), 7.17 (d, J=8.2 Hz, 2H), 6.77 (d, J=8.1 Hz, 2H), 4.71 (s, 2H), 4.45 (s, 2H).

### 2-(4-hydroxybenzyl)-2,3-dihydro-1H-pyrrolo[3,4-c]pyridin-1-one (43)

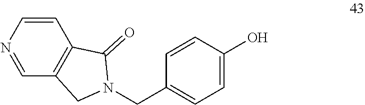 [SMILES: O=C1c2ccncc2CN1Cc1ccc(O)cc1]

The title compound 43 was prepared according to general procedure A as a yellow solid (11 mg, 21.5%). MS (ESI) m/z 241 [M+H]⁺. ¹H NMR (400 MHz, DMSO-d₆) δ 9.40 (s, 1H), 8.92 (s, 1H), 8.73 (d, J=5.0 Hz, 1H), 7.63 (d, J=5.0 Hz, 11H), 7.10 (d, J=8.2 Hz, 2H), 6.73 (d, J=8.2 Hz, 2H), 4.60 (s, 2H), 4.40 (s, 2H).

### 2-((1H-benzo[d][1,2,3]triazol-5-yl)methyl)-3-methylisoindolin-1-one (44)

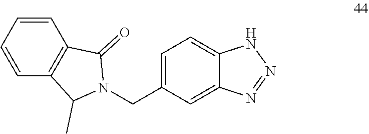

The title compound 44 was prepared according to general procedure C as a white solid (8 mg, 28%). MS (ESI) m/z 279 [M+H]⁺. ¹H NMR (400 MHz, DMSO-d₆) δ 15.63 (s, 1H), 7.92-7.80 (m, 2H), 7.75 (d, J=7.5 Hz, 1H), 7.66-7.55 (m, 2H), 7.51 (t, J=7.2 Hz, 1H), 7.36 (d, J=8.6 Hz, 1H), 5.17 (d, J=15.3 Hz, 11H), 4.62 (d, J=15.3 Hz, 1H), 4.48 (q, J=6.7 Hz, 11H), 1.41 (d, J=6.6 Hz, 3H).

### 2-(1-(4-hydroxyphenyl) ethyl) isoindolin-1-one (45)

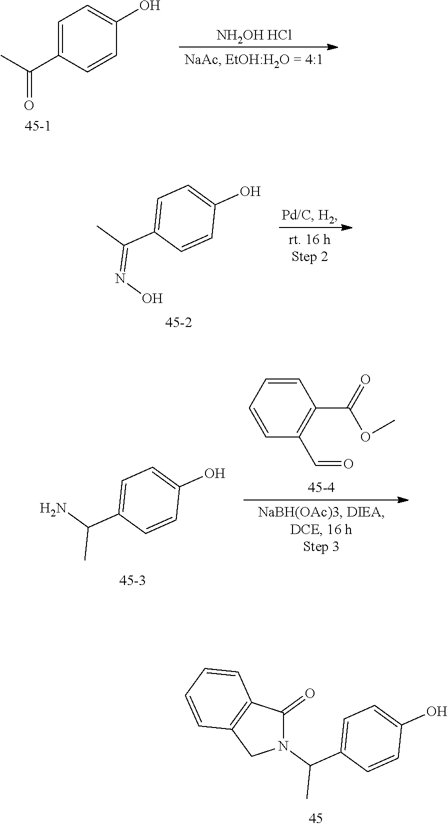

### Step 1. (Z)-1-(4-hydroxyphenyl)ethan-1-one oxime (45-2)

To a solution of 1-(4-hydroxyphenyl)ethan-1-one (3.5 g, 0.026 mol) in EtOH: H₂O=4:1 (50 mL) was added NaOAc (4.22 g, 0.05 mol) and hydroxylamine hydrochloride (2.86 g, 0.41 mol). The reaction mixture was stirred at r.t. for 16 h. Water was added and then the mixture was extracted with EtOAc, and the organic layer was collected. Then the crude was purified by silica gel column (EtOAc:PE=1:3) to give (Z)-1-(4-hydroxyphenyl)ethan-1-one oxime (3 g, 77.2%) as a white solid. MS (ESI) m/z 152 [M+H]⁺.

### Step 2. 4-(1-aminoethyl) phenol (45-3)

To a solution of (Z)-1-(4-hydroxyphenyl)ethan-1-one oxime (500 mg, 3.3 mmol) in MeOH (10 mL) were added Pd/C (2.5 g, 6.6 mmol) under the condition of hydrogen flow stirred 16 h at r.t. After the reaction was completed, the reaction mixture was filtered and the filtrate was collected. The solvent was removed in vacuum to give 4-(1-aminoethyl) phenol (400 mg, 88.8%) as a gray solid. MS (ESI) m/z 138 [M+H]⁺.

### Step 3. 2-(1-(4-hydroxyphenyl) ethyl) isoindolin-1-one (45)

To a solution of 4-(1-aminoethyl)phenol (80 mg, 0.6 mmol) in DCE (5 mL) were added methyl 2-formylbenzoate (670 mg, 4.1 mmol), DIPEA (151 mg, 1.7 mmol) and Sodium triacetoxyborohydride (618 mg, 2.9 mmol). The reaction mixture was stirred at r.t. for 16 h. After the reaction completed, the solvent was removed in vacuum and the crude was purified by prep-HPLC to give 2-(1-(4-hydroxyphenyl)ethyl) isoindolin-1-one (41 mg, 27.6%) as a white solid. MS (ESI) m/z 254 [M+H]⁺. ¹H NMR (400 MHz, DMSO-d₆) δ 9.40 (s, 1H), 7.69 (d, J=7.5 Hz, 1H), 7.59-7.52 (m, 2H), 7.48 (t, J=7.2 Hz, 1H), 7.14 (d, J=8.4 Hz, 2H), 6.73 (d, J=8.4 Hz, 2H), 5.45 (q, J=7.1 Hz, 1H), 4.47 (d, J=17.7 Hz, 1H), 4.01 (d, J=17.7 Hz, 1H), 1.58 (d, J=7.1 Hz, 3H).

### 6-((1-methyl-3-oxo-1,3-dihydro-2H-indazol-2-yl)methyl)benzo[d]oxazol-2(3H)-one (46)

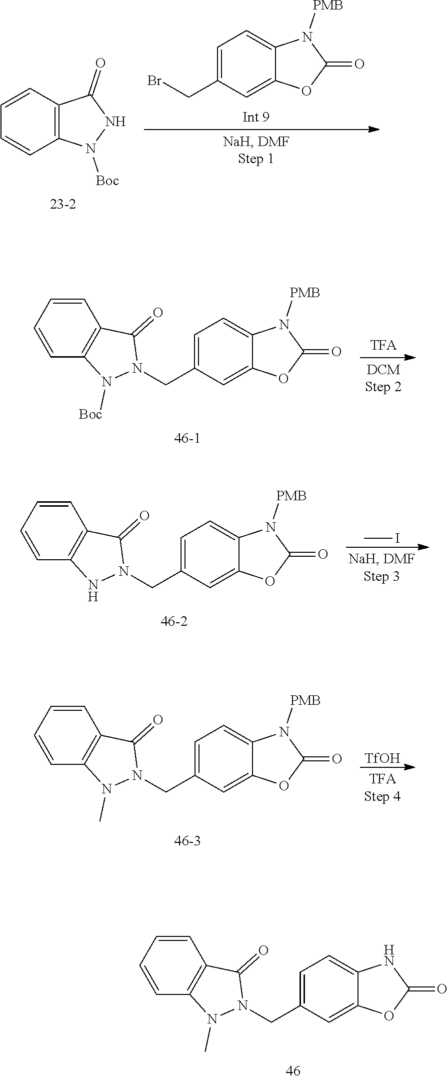

### Step 1. tert-butyl 2-((3-(4-methoxybenzyl)-2-oxo-2,3-dihydrobenzo[d]oxazol-6-yl)methyl)-3-oxo-2,3-dihydro-1H-indazole-1-carboxylate (46-1)

To a solution of tert-butyl 3-oxo-2,3-dihydro-1H-indazole-1-carboxylate (81 mg, 0.34 mmol) in DMF (1 mL) was added NaH (8 mg, 0.34 mmol) at 0° C. under N₂. The mixture was stirred for 30 min. 6-(bromomethyl)-3-(4-methoxybenzyl)benzo[d]oxazol-2(3H)-one (120 mg, 0.34 mmol) was added and the reaction allowed to warm to r.t. This suspension was allowed to stir for an additional 2 h. Water was added at 0° C. and then extracted with EtOAc. The organic layer was separated, dried over anhydrous Na₂SO₄ and concentrated under vacuum. The residue was purified by prep TLC (PE/EtOAc=1.5/1) to give tert-butyl 2-((3-(4-methoxybenzyl)-2-oxo-2,3-dihydrobenzo[d]oxazol-6-yl)methyl)-3-oxo-2,3-dihydro-1H-indazole-1-carboxylate (80 mg, 46%) as a yellow solid. MS (ESI) m/z 502 [M+H]⁺.

### Step 2. 3-(4-methoxybenzyl)-6-((3-oxo-1,3-dihydro-2H-indazol-2-yl)methyl)benzo[d]oxazol-2(3H)-one (46-2)

TFA (1 mL) was added to a stirred solution of tert-butyl 2-((3-(4-methoxybenzyl)-2-oxo-2,3-dihydrobenzo[d]oxazol-6-yl)methyl)-3-oxo-2,3-dihydro-1H-indazole-1-carboxylate (80 mg, 0.16 mmol) in DCM (2 mL). The mixture was stirred at rt for 1 h. The mixture was concentrated in vacuo to give 3-(4-methoxybenzyl)-6-((3-oxo-1,3-dihydro-2H-indazol-2-yl)methyl)benzo[d]oxazol-2(3H)-one (60 mg, 93%) as a white solid. MS (ESI) m/z 402 [M+H]⁺.

### Step 3. 3-(4-methoxybenzyl)-6-((1-methyl-3-oxo-1,3-dihydro-2H-indazol-2-yl)methyl) benzo[d]oxazol-2(3H)-one (46-3)

To a solution of 3-(4-methoxybenzyl)-6-((3-oxo-1,3-dihydro-2H-indazol-2-yl)methyl) benzo[d]oxazol-2(3H)-one (60 mg, 0.14 mmol) in DMF (1 mL) was added NaH (4 mg, 0.14 mmol) at 0° C. under N₂. The mixture was stirred for 30 min. Iodomethane (21 mg, 0.14 mmol) was added and the reaction allowed to warm to r.t. This suspension was allowed to stir for an additional 2 h. Water was added at 0° C. and then extracted with EtOAc. The organic layer was separated, dried over anhydrous Na₂SO₄ and concentrated under vacuum. The residue was purified by prep TLC (PE/EtOAc=1/1) to give 3-(4-methoxybenzyl)-6-((1-methyl-3-oxo-1,3-dihydro-2H-indazol-2-yl)methyl)benzo[d]oxazol-2(3H)-one (40 mg, 64%) as a yellow solid. MS (ESI) m/z 416 [M+H]⁺.

### Step 4. 6-((1-methyl-3-oxo-1,3-dihydro-2H-indazol-2-yl)methyl)benzo[d]oxazol-2(3H)-one (46)

TfOH (0.1 mL) was added to a stirred solution of 3-(4-methoxybenzyl)-6-((1-methyl-3-oxo-1,3-dihydro-2H-indazol-2-yl)methyl)benzo[d]oxazol-2(3H)-one (40 mg, 0.09 mmol) in TFA (1 mL). The mixture was stirred at 70° C. for 0.5 h. The mixture was concentrated in vacuo and purified by prep HPLC to give 6-((1-methyl-3-oxo-1,3-dihydro-2H-indazol-2-yl)methyl)benzo[d]oxazol-2(3H)-one (7 mg, 22%) as a white solid. MS (ESI) m/z 296 [M+H]⁺. ¹H NMR (400 MHz, DMSO-d₆) δ 11.58 (s, 1H), 7.72 (d, J=7.8 Hz, 1H), 7.58 (t, J=7.7 Hz, 1H), 7.46 (d, J=8.3 Hz, 1H), 7.23-7.14 (m, 2H), 7.09-6.98 (m, 2H), 5.07 (s, 2H), 3.26 (s, 3H).

### 6-((5-morpholino-1-oxoisoindolin-2-yl)methyl)benzo[d]oxazol-2(3H)-one (47)

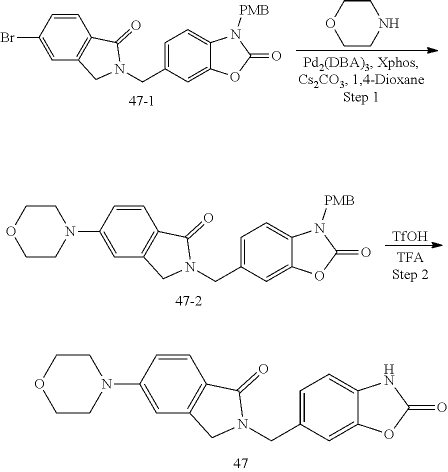

The intermediate 47-1 was prepared according to the procedure described for Int 7.

### Step 1. 3-(4-methoxybenzyl)-6-((5-morpholino-1-oxoisoindolin-2-yl)methyl)benzo[d]oxazol-2(3H)-one (47-2)

To a solution of 6-((5-bromo-1-oxoisoindolin-2-yl)methyl)-3-(4-methoxybenzyl) benzo[d]oxazol-2(3H)-one (150 mg, 0.31 mmol) in 1,4-Dioxane (2.5 mL) was added morpholine (41 mg, 0.46 mmol), Cs₂CO₃ (204 mg, 0.62 mmol), Xphos (30 mg, 0.06 mmol), Pd₂(dba)₃ (57 mg, 0.06 mmol) under N₂. The mixture was stirred at 100° C. for 14 h. The mixture was purified by prep TLC (DCM/MeOH=35/1) to give 3-(4-methoxybenzyl)-6-((5-morpholino-1-oxoisoindolin-2-yl)methyl)benzo[d]oxazol-2(3H)-one (47 mg, 30%) as a yellow solid. MS (ESI) m/z 486 [M+H]⁺.

### Step 2. 6-((5-morpholino-1-oxoisoindolin-2-yl)methyl)benzo[d]oxazol-2(3H)-one (47)

TfOH (0.1 mL) was added to a stirred solution of 3-(4-methoxybenzyl)-6-((5-morpholino-1-oxoisoindolin-2-yl)methyl)benzo[d]oxazol-2(3H)-one (47 mg, 0.09 mmol) in TFA (1 mL). The mixture was stirred at 70° C. for 0.5 h. The mixture was concentrated in vacuo and purified by prep HPLC to give 6-((5-morpholino-1-oxoisoindolin-2-yl)methyl)benzo[d]oxazol-2(3H)-one (3 mg, 6%) as a white solid. MS (ESI) m/z 366 [M+H]⁺. ¹H NMR (400 MHz, DMSO-d₆) δ 11.60 (s, 1H), 7.53 (d, J=8.3 Hz, 1H), 7.18 (s, 1H), 7.06-7.02 (m, 4H), 4.66 (s, 2H), 4.25 (s, 2H), 3.73 (t, J=4.8 Hz, 4H), 3.21 (t, J=4.9 Hz, 4H).

### 6-((5-methyl-1-oxoisoindolin-2-yl)methyl)benzo[d]oxazol-2(3H)-one (48)

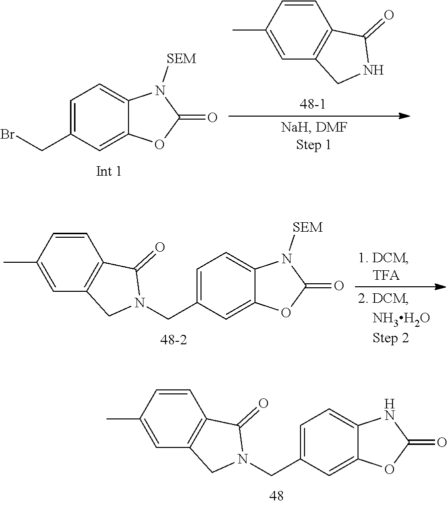 [SMILES: C[Si](C)(C)CCOCn1c(=O)oc2cc(CBr)ccc21 ; Cc1ccc2c(c1)CNC2=O ; Cc1ccc2c(c1)CN(Cc1ccc3c(c1)oc(=O)n3COCC[Si](C)(C)C)C2=O ; Cc1ccc2c(c1)CN(Cc1ccc3[nH]c(=O)oc3c1)C2=O]

### Step 1. 6-((5-methyl-1-oxoisoindolin-2-yl)methyl)-3-((2-(trimethylsilyl)ethoxy)methyl) benzo[d]oxazol-2(3H)-one (48-2)

To a solution of 5-methylisoindolin-1-one (33 mg, 0.22 mmol) in DMF (1 mL) was added NaH (8 mg, 0.34 mmol) at 0° C. under N₂. The mixture was stirred for 30 min. 6-(bromomethyl)-3-((2-(trimethylsilyl)ethoxy)methyl)benzo[d]oxazol-2(3H)-one (80 mg, 0.22 mmol) was added and the reaction allowed to warm to r.t. This suspension was allowed to stir for an additional 2 h. Water was added at 0° C. and then extracted with EtOAc. The organic layer was separated, dried over anhydrous Na₂SO₄ and concentrated under vacuum. The residue was purified by prep TLC (PE/EtOAc=1.5/1) to give 6-((5-methyl-1-oxoisoindolin-2-yl)methyl)-3-((2-(trimethylsilyl) ethoxy)methyl)benzo[d]oxazol-2(3H)-one (60 mg, 63%) as a yellow solid. MS (ESI) m/z 425 [M+H]⁺.

### Step 2. 6-((5-methyl-1-oxoisoindolin-2-yl)methyl)benzo[d]oxazol-2(3H)-one (48)

To a solution of 6-((5-methyl-1-oxoisoindolin-2-yl)methyl)-3-((2-(trimethylsilyl) ethoxy)methyl) benzo[d]oxazol-2(3H)-one (60 mg, 0.14 mmol) in DCM (3 mL) was added TFA (1 mL). The mixture was stirred for 3 h and then concentrated under vacuum. The residue was taken in DCM (3 mL) and ammonium hydroxide (1 mL) was added at 0° C. This suspension was allowed to stir for an additional 1 h. The mixture was concentrated and purified by prep HPLC to give 6-((5-methyl-1-oxoisoindolin-2-yl)methyl)benzo[d]oxazol-2(3H)-one (11 mg, 24%) as a white solid. MS (ESI) m/z 295 [M+H]⁺. ¹H NMR (400 MHz, DMSO-d₆) δ 11.61 (s, 1H), 7.60 (d, J=7.7 Hz, 1H), 7.35 (s, 1H), 7.31 (d, J=8.3 Hz, 1H), 7.21 (s, 1H), 7.08-7.04 (m, 2H), 4.70 (s, 2H), 4.31 (s, 2H), 2.40 (s, 3H).

### 6-((5-methoxy-1-oxoisoindolin-2-yl)methyl)benzo[d]oxazol-2(3H)-one (49)

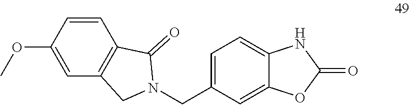

The title compound 49 was prepared according to the procedure described for compound 48 as a white solid (22 mg, 61%). MS (ESI) m/z 311 [M+H]+ 0.1H NMR (400 MHz, DMSO-d₆) δ 11.61 (s, 1H), 7.62 (d, J=8.4 Hz, 1H), 7.20 (s, 1H), 7.11 (d, J=2.2 Hz, 1H), 7.07-7.01 (m, 3H), 4.68 (s, 2H), 4.30 (s, 2H), 3.81 (s, 3H).

### 6-((1-methyl-3-oxo-5-(pyrrolidin-1-yl)isoindolin-2-yl)methyl)benzo[d]oxazol-2(3H)-one (50) and 6-((3-methyl-1-oxo-5-(pyrrolidin-1-yl)isoindolin-2-yl)methyl)benzo[d]oxazol-2(3H)-one (51)

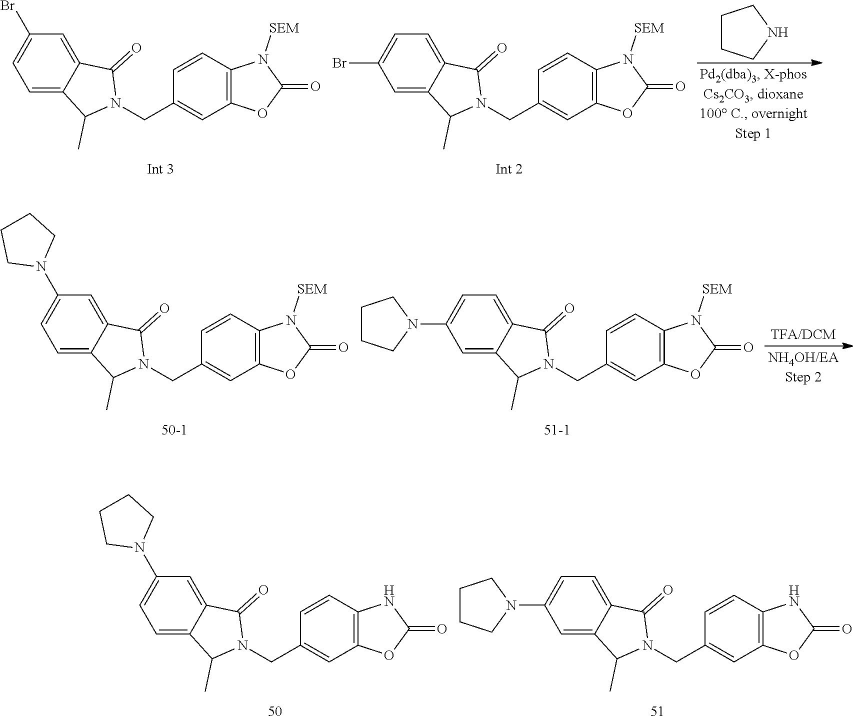

### Step 1. Mixture of 6-((1-methyl-3-oxo-5-(pyrrolidin-1-yl)isoindolin-2-yl)methyl)-3-((2-(trimethylsilyl)ethoxy)methyl)benzo[d]oxazol-2(3H)-one (50-1) and 6-((3-methyl-1-oxo-5-(pyrrolidin-1-yl)isoindolin-2-yl)methyl)-3-((2-(trimethylsilyl)ethoxy)methyl)benzo[d]oxazol-2(3H)-one (51-1)

To a solution of mixture of Int 3 and Int 2 (400 mg, 0.79 mmol) in DMF (6 mL) were added Pd₂(dba)₃ (36 mg, 0.04 mmol), X-phos (38 mg, 0.08 mmol), Cs₂CO₃ (518 mg, 1.59 mmol) and pyrrolidine (169 mg, 2.38 mmol) at r.t. under N₂. The resulting mixture was stirred for 15 h at 100° C. The mixture was diluted with EtOAc (30 mL) and washed with water (3×20 mL). The organic layer was separated, dried over anhydrous Na₂SO₄ and concentrated under vacuum. The residue was purified by silica gel flash column chromatography (PE/EtOAc=1/3) to afford the mixture of title compounds (80 mg, 20.4%) as a yellow oil. MS (ESI) m/z 494 [M+H]⁺.

### Step 2. 6-((1-methyl-3-oxo-5-(pyrrolidin-1-yl)isoindolin-2-yl)methyl)benzo[d]oxazol-2(3H)-one (50) and 6-((3-methyl-1-oxo-5-(pyrrolidin-1-yl)isoindolin-2-yl)methyl)benzo[d]oxazol-2(3H)-one (51)

To a solution of mixture of 50-1 and 51-1 (80 mg, 0.16 mmol) in DCM (5 mL) was added trifluoroacetic acid (2 mL). The mixture was stirred at r.t. for 2h, and then concentrated under vacuum. The residue was dissolved with DCM (2 mL), and NH₄OH (1 mL) was added dropwise. The mixture was stirred at r.t. for 1 h. The reaction mixture was concentrated, and the residue was purified by prep-HPLC to afford two compounds: compound 50 and compound 51. One of these two compounds was a white solid (3 mg, 10.2%), with RT=5.19 min (HPLC), MS (ESI) m/z 363 [M+H]⁺. ¹H NMR (400 MHz, DMSO-d₆) δ 7.32 (d, J=8.4 Hz, 1H), 7.16 (s, 1H), 7.05-6.99 (m, 2H), 6.78-6.73 (m, 2H), 4.99 (d, J=15.2 Hz, 1H), 4.34 (d, J=15.2 Hz, 1H), 4.23 (q, J=6.4 Hz, 1H), 3.28-3.25 (m, 4H), 2.01-1.96 (m, 2H), 1.30 (d, J=6.4 Hz, 3H). The other one of the two compounds was a white solid (3 mg, 10.2%), with RT=5.41 min (HPLC), MS (ESI) m/z 363 [M+H]⁺. ¹H NMR (400 MHz, DMSO-d₆) δ 7.46 (d, J=9.2 Hz, 1H), 7.16 (s, 1H), 7.05-7.00 (m, 2H), 6.58-6.51 (m, 2H), 4.93 (d, J=15.2 Hz, 1H), 4.30 (d, J=15.2 Hz, 1H), 4.22 (q, J=6.4 Hz, 1H), 3.31-3.24 (m, 4H), 1.99-1.93 (m, 4H), 1.32 (d, J=6.4 Hz, 3H).

### 6-((1,1-dimethyl-3-oxoisoindolin-2-yl) methyl) benzo[d]oxazol-2(3H)-one (52)

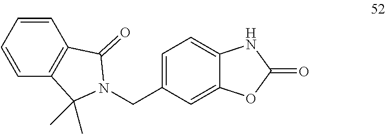

The title compound 52 was prepared according to general procedure B as an off-white solid (58 mg, 42.2%). MS (ESI) m/z 309 [M+H]⁺. ¹H NMR (400 MHz, DMSO-d₆) δ 11.58 (s, 1H), 7.72 (d, J=7.6 Hz, 1H), 7.67-7.60 (m, 2H), 7.49 (t, J=7.2 Hz, 1H), 7.31 (s, 1H), 7.19 (dd, J=8.0, 1.3 Hz, 1H), 7.02 (d, J=8.0 Hz, 1H), 4.69 (s, 2H), 1.36 (s, 6H).

### 6-((1-ethyl-3-oxoisoindolin-2-yl) methyl) benzo[d]oxazol-2(3H)-one (53)

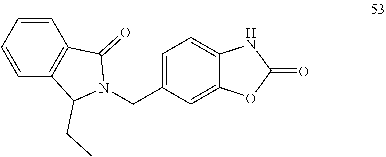

The title compound 53 was prepared according to general procedure B as an off-white solid (20 mg, 14%). MS (ESI) m/z 309 [M+H]⁺. ¹H NMR (400 MHz, DMSO-d₆) δ 11.60 (s, 1H), 7.72 (d, J=7.5 Hz, 1H), 7.63-7.48 (m, 3H), 7.26 (s, 1H), 7.11 (dd, J=8.0, 1.2 Hz, 1H), 7.04 (d, J=8.0 Hz, 1H), 5.06 (d, J=15.1 Hz, 1H), 4.51 (t, J=4.0 Hz, 1H), 4.29 (d, J=15.1 Hz, 1H), 2.05-1.99 (m, 2H), 0.36 (t, J=7.3 Hz, 3H).

### 6-((1-methyl-3-oxoisoindolin-2-yl)methyl)benzo[d]oxazol-2(3H)-one (54)

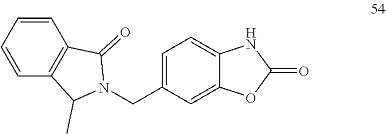

The title compound 54 was prepared according to general procedure B as a white solid (8 mg, 21%). MS (ESI) m/z 295 [M+H]⁺. ¹H NMR (400 MHz, DMSO-d₆) δ 11.29 (s, 1H), 7.72 (d, J=7.5 Hz, 1H), 7.65-7.55 (m, 2H), 7.50 (t, J=7.2 Hz, 1H), 7.24 (s, 1H), 7.09 (d, J=8.0 Hz, 1H), 7.04 (d, J=7.9 Hz, 1H), 5.01 (d, J=15.2 Hz, 1H), 4.49-4.38 (m, 2H), 1.39 (d, J=6.7 Hz, 3H).

### (R)-6-((1-methyl-3-oxoisoindolin-2-yl)methyl)benzo[d]oxazol-2(3H)-one (55)

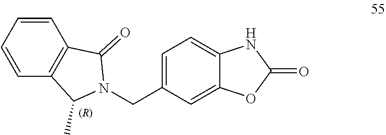

The title compound 55 was prepared according to general procedure B using (R)-3-methylisoindolin-1-one as a starting material to afford a white solid (52 mg, 28%). MS (ESI) m/z 295 [M+H]⁺. ¹H NMR (400 MHz, CD₃OD) δ 7.81 (d, J=7.5 Hz, 1H), 7.61 (t, J=7.4 Hz, 1H), 7.56-7.47 (m, 2H), 7.21 (s, 1H), 7.16 (d, J=8.0 Hz, 1i), 7.04 (d, J=8.0 Hz, 1H), 5.16 (d, J=15.1 Hz, 1H), 4.54-4.46 (m, 2H), 1.46 (d, J=6.8 Hz, 3H). CHIRAL HPLC: (column: CHIRALPAK IA-3; column size: 4.6*50 mm, 3 μm; mobile phase: MTBE (0.1% DEA):(MeOH:DCM=1:1)=85:15; flow: 1.0 mL/min) RT=1.704 min.

### (S)-6-((1-methyl-3-oxoisoindolin-2-yl)methyl)benzo[d]oxazol-2(3H)-one (56)

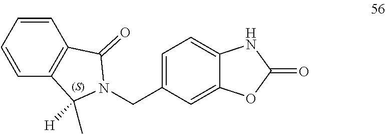

Chiral separation of compound 54 by chiral prep-HPLC (column: CHIRALPAK IA; column size: 2 cm×25 cm, 5 μm; mobile phase A: MTBE; mobile phase B: MeOH:DCM) was conducted to afford two enantiomers: compound 55 and 56. Compound 56 was an off-white solid, with RT=2.104 min (CHIRAL HPLC, column: CHIRALPAK IA-3; column size: 4.6*50 mm, 3 μm; mobile phase: MTBE (0.1% DEA):(MeOH:DCM=1:1)=85:15; flow: 1.0 mL/min). MS (ESI) m/z 295 [M+H]⁺. ¹H NMR (400 MHz, CD₃OD) δ 7.81 (d, J=7.6 Hz, 1H), 7.61 (t, J=7.5 Hz, 1H), 7.56-7.47 (m, 2H), 7.21 (s, 1H), 7.16 (d, J=8.0 Hz, 1H), 7.04 (d, J=8.1 Hz, 1H), 5.16 (d, J=15.2 Hz, 1H), 4.54-4.46 (m, 2H), 1.46 (d, J=6.8 Hz, 3H).

### 6-((3-methyl-1-oxo-5-(1H-pyrazol-4-yl)isoindolin-2-yl)methyl)benzo[d]oxazol-2(3H)-one (57) and 6-((1-methyl-3-oxo-5-(1H-pyrazol-4-yl)isoindolin-2-yl)methyl) benzo[d]oxazol-2(3H)-one (58)

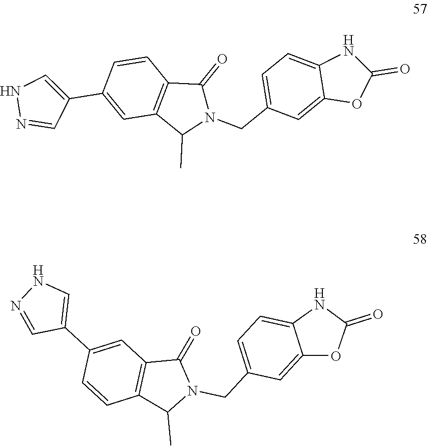

The title compounds 57 and 58 were prepared according to the procedure described for compounds 50 and 51. After pre-HPLC separation, two compounds were obtained: compound 57 and compound 58. One of the two compounds was a white solid (13 mg, 15.3%), with RT=2.98 min (HPLC), MS (ESI) m/z 361 [M+H]⁺. ¹H NMR (400 MHz, DMSO-d₆) δ 13.03 (s, 1H), 11.68 (s, 1H), 8.16 (bs, 2H), 7.83 (s, 1H), 7.75 (dd, J=8.0, 1.2 Hz, 1H), 7.67 (d, J=7.6 Hz, 1H), 7.24 (d, J=1.5 Hz, 1H), 7.09 (dd, J=8.0, 2.0 Hz, 1H), 7.04 (d, J=8.0 Hz, 1H), 4.99 (d, J=15.3 Hz, 1H), 4.48-4.37 (m, 2H), 1.43 (d, J=6.7 Hz, 3H). The other one of the two compounds was a white solid (16 mg, 18.8%), with RT=3.27 min (HPLC), MS (ESI) m/z 361 [M+H]⁺. ¹H NMR (400 MHz, DMSO-d₆) δ 12.91 (s, 1H), 11.74 (s, 1H), 8.18 (bs, 2H), 7.93 (s, 1H), 7.85 (dd, J=7.9, 1.6 Hz, 1H), 7.55 (d, J=7.9 Hz, 1H), 7.25 (s, 1H), 7.09 (d, J=8.0 Hz, 1H), 7.04 (d, J=8.0 Hz, 1H), 5.03 (d, J=15.2 Hz, 1H), 4.50-4.35 (m, 2H), 1.40 (d, J=6.7 Hz, 3H).

### 6-((5-(3-methoxypyrrolidin-1-yl)-3-methyl-1-oxoisoindolin-2-yl)methyl)benzo[d]oxazol-2(3H)-one (59) and 6-((5-(3-methoxypyrrolidin-1-yl)-1-methyl-3-oxoisoindolin-2-yl) methyl)benzo[d]oxazol-2(3H)-one (60)

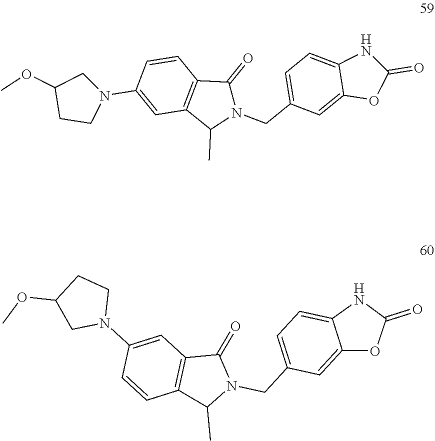

The title compounds 59 and 60 were prepared according to the procedure described for compounds 50 and 51. After pre-HPLC separation, two compounds were obtained: compound 59 and compound 60. One of the two compounds was a white solid (5 mg, 7.4%), with RT=4.21 min (HPLC), MS (ESI) m/z 394 [M+H]⁺. ¹H NMR (400 MHz, DMSO-d₆) δ 7.47 (d, J=9.0 Hz, 1H), 7.16 (s, 1H), 7.06-6.96 (m, 2H), 6.62-6.57 (m, 2H), 4.94 (d, J=15.2 Hz, 1H), 4.32 (d, J=15.2 Hz, 1H), 4.26 (q, J=6.4 Hz, 1H), 4.11-4.07 (m, 1H), 3.49-3.27 (m, 7H), 2.12-2.03 (m, 2H), 1.35 (d, J=6.7 Hz, 3H). The other one of the two compounds was a white solid (4 mg, 6%), with RT=4.34 min (HPLC), MS (ESI) m/z 394 [M+H]⁺. ¹H NMR (400 MHz, DMSO-d₆) δ 7.33 (d, J=8.2 Hz, 1H), 7.18 (s, 1H), 7.08-6.98 (m, 2H), 6.81-6.72 (m, 2H), 4.99 (d, J=15.2 Hz, 1H), 4.36 (d, J=15.2 Hz, 1H), 4.29 (q, J=6.4 Hz, 1H), 4.13-4.05 (m, 1H), 3.48-3.29 (m, 4H), 3.27 (s, 3H), 2.14-2.02 (m, 2H), 1.32 (d, J=6.6 Hz, 3H).

### Mixture of 6-((3-methyl-5-(oxazol-5-yl)-1-oxoisoindolin-2-yl)methyl)benzo[d]oxazol-2(3H)-one (61) and 6-((1-methyl-5-(oxazol-5-yl)-3-oxoisoindolin-2-yl)methyl)benzo[d]oxazol-2(3H)-one (62)

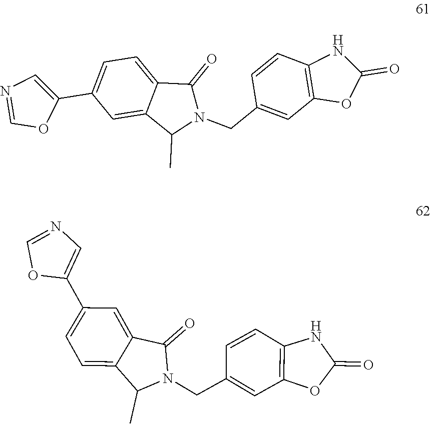

A mixture of compound 61 and 62 was prepared according to the procedure described for compounds 50 and 51 as a white solid (3 mg, 5.5%). MS (ESI) m/z 362 [M+H]⁺. ¹H NMR (400 MHz, DMSO-d₆) δ 11.60 (s, 1H), 8.52 (s, 0.5H), 8.49 (s, 0.5H), 8.04 (s, 1H), 7.90-7.95 (m, 1H), 7.88-7.80 (m, 2H), 7.70 (d, J=8.0 Hz, 1H), 7.26 (s, 1H), 7.13-7.09 (m, 1H), 7.05 (d, J=8.0 Hz, 1H), 5.04 (d, J=15.2 Hz, 0.5H), 5.00 (d, J=15.2 Hz, 0.5H), 4.58-4.38 (m, 2H), 1.44 (d, J=6.4 Hz, 1.5H), 1.42 (d, J=6.4 Hz, 1.5H).

### 6-((3-methyl-5-morpholino-1-oxoisoindolin-2-yl)methyl)benzo[d]oxazol-2(3H)-one (63) and 6-((1-methyl-5-morpholino-3-oxoisoindolin-2-yl)methyl)benzo[d]oxazol-2(3H)-one (64)

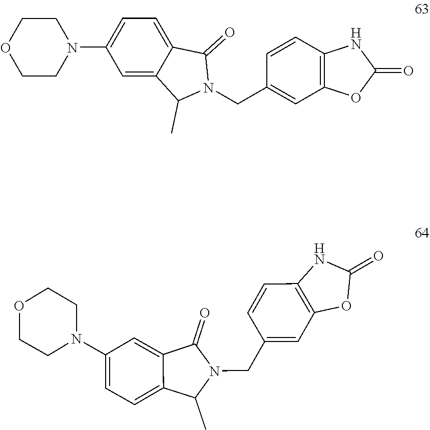

The title compounds 63 and 64 were prepared according to the procedure described for compounds 50 and 51. After pre-HPLC separation, two compounds were obtained: compound 63 and compound 64. One of the two compounds was a white solid (8 mg, 7.9%), with RT=3.54 min (HPLC), MS (ESI) m/z 380 [M+H]⁺. ¹H NMR (400 MHz, DMSO-d₆) δ 11.58 (s, 1H), 7.52 (d, J=8.4 Hz, 1H), 7.19 (s, 1H), 7.08-6.99 (m, 4H), 4.95 (d, J=15.2 Hz, 1H), 4.35 (d, J=15.2 Hz, 1H), 4.30 (q, J=6.4 Hz, 1H), 3.73 (t, J=4.9 Hz, 4H), 3.25-3.19 (m, 4H), 1.36 (d, J=6.6 Hz, 3H). The other one of the two compounds was a white solid (9 mg, 8.9%), with RT=3.59 min (HPLC), MS (ESI) m/z 380 [M+H]⁺. 11H NMR (400 MHz, DMSO-d₆) δ 11.72 (s, 1H), 7.41 (d, J=8.4 Hz, 1H), 7.24-7.16 (m, 3H), 7.11-6.98 (m, 2H), 5.00 (d, J=15.2 Hz, 1H), 4.37 (d, J=15.2 Hz, 1H), 4.32 (q, J=6.4 Hz, 11H), 3.75 (t, J=4.8 Hz, 4H), 3.16 (t, J=4.9 Hz, 4H), 1.34 (d, J=6.8 Hz, 3H).

### 6-((3-methyl-1-oxo-5-(pyridin-3-ylamino)isoindolin-2-yl)methyl)benzo[d]oxazol-2(3H)-one (65) and 6-((1-methyl-3-oxo-5-(pyridin-3-ylamino)isoindolin-2-yl)methyl) benzo[d]oxazol-2(3H)-one (66)

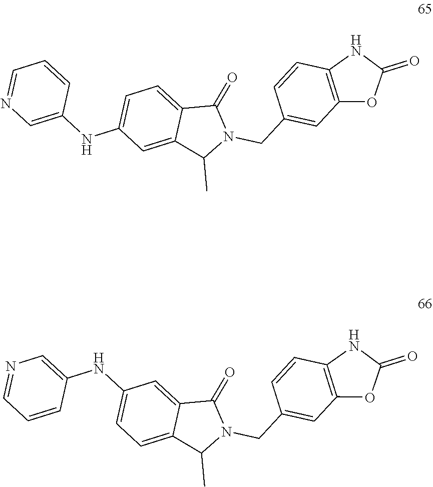

The title compounds 65 and 66 were prepared according to the procedure described for compounds 50 and 51. After pre-HPLC separation, two compounds were obtained: compound 65 and compound 66. One of the two compounds was a white solid (15 mg, 11%), with RT=2.09 min (HPLC), MS (ESI) m/z 387 [M+H]⁺. ¹H NMR (400 MHz, DMSO-d₆) δ 8.82 (s, 1H), 8.42 (d, J=2.7 Hz, 1H), 8.13 (dd, J=4.7, 1.4 Hz, 1H), 7.62-7.51 (m, 2H), 7.29 (dd, J=8.3, 4.6 Hz, 1H), 7.21 (s, 1H), 7.19 (d, J=2.0 Hz, 1H), 7.14-7.02 (m, 3H), 4.98 (d, J=15.2 Hz, 1H), 4.39-4.31 (m, 2H), 1.36 (d, J=6.7 Hz, 3H). The other one of the two compounds was a white solid (12 mg, 8.8%), with RT=2.21 min (HPLC), MS (ESI) m/z 387 [M+H]⁺. ¹H NMR (400 MHz, DMSO-d₆) δ 8.59 (s, 1H), 8.38 (d, J=2.7 Hz, 1H), 8.09 (d, J=3.6 Hz, 1H), 7.61-7.48 (m, 11H), 7.45 (d, J=8.1 Hz, 1H), 7.34 (d, J=2.1 Hz, 1H), 7.31-7.26 (m, 2H), 7.22 (s, 1H), 7.14-7.01 (m, 2H), 4.99 (d, J=15.2 Hz, 1H), 4.43-4.30 (m, 2H), 1.37 (d, J=6.6 Hz, 3H).

### 6-((1-oxo-3-phenylisoindolin-2-yl)methyl)benzo[d]oxazol-2(3H)-one (67)

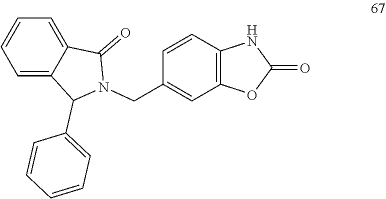

The title compound 67 was prepared according to the procedure described for compound 48 as a white solid (21 mg, 21.8%). MS (ESI) m/z 357 [M+H]⁺. ¹H NMR (400 MHz, DMSO-d₆) δ 11.59 (s, 1H), 7.84-7.75 (m, 1H), 7.57-7.47 (m, 2H), 7.41-7.29 (m, 3H), 7.26-7.17 (m, 1H), 7.17-7.09 (m, 2H), 7.01-6.98 (m, 2H), 6.90 (dd, J=8.0, 1.6 Hz, 1H), 5.53 (s, 1H), 5.07 (d, J=15.1 Hz, 11H), 3.87 (d, J=15.1 Hz, 11H).

The intermediate 67-3 was prepared as follows:

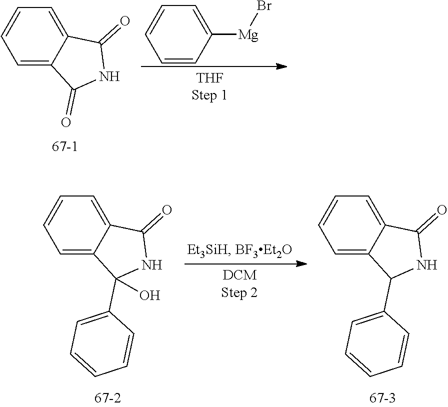

### Step 1. 3-hydroxy-3-phenylisoindolin-1-one (67-2)

To a solution of isoindoline-1,3-dione (2 g, 13.6 mmol) in THF (20 mL) was added phenylmagnesium bromide (27.2 mL, 27.2 mmol) at 0′. The resulting mixture was stirred for 3 h at rt, diluted with EtOAc (30 mL) and washed with water (3×20 mL). The organic layer was separated, dried over anhydrous Na₂SO₄ and concentrated under vacuum to afford the title compound (3.06 g, crude) as a yellow solid. MS (ESI) m/z 226 [M+H]⁺.

### Step 2. 3-phenylisoindolin-1-one (67-3)

To a solution of 3-hydroxy-3-phenylisoindolin-1-one (3.06 g, 13.6 mmol) in DCM (10 mL) were added triethylsilane (7.9 g, 68 mmol) and borontrifluoridediethyletherate (3.9 g, 27.2 mmol). The resulting mixture was stirred for 3 h at rt, diluted with EtOAc (30 mL) and washed with water (3×20 mL). The organic layer was separated, dried over anhydrous Na₂SO₄ and concentrated under vacuum. The residue was purified by TLC (PE/EA=1/3) to afford the title compound (1.8 g, 63.3%) as a yellow solid. MS (ESI) m/z 210 [M+H]⁺.

### N-(3-methyl-1-oxo-2-((2-oxo-2,3-dihydrobenzo[d]oxazol-6-yl)methyl)isoindolin-5-yl) acetamide (68) and N-(1-methyl-3-oxo-2-((2-oxo-2,3-dihydrobenzo[d]oxazol-6-yl)methyl)isoindolin-5-yl)acetamide (69)

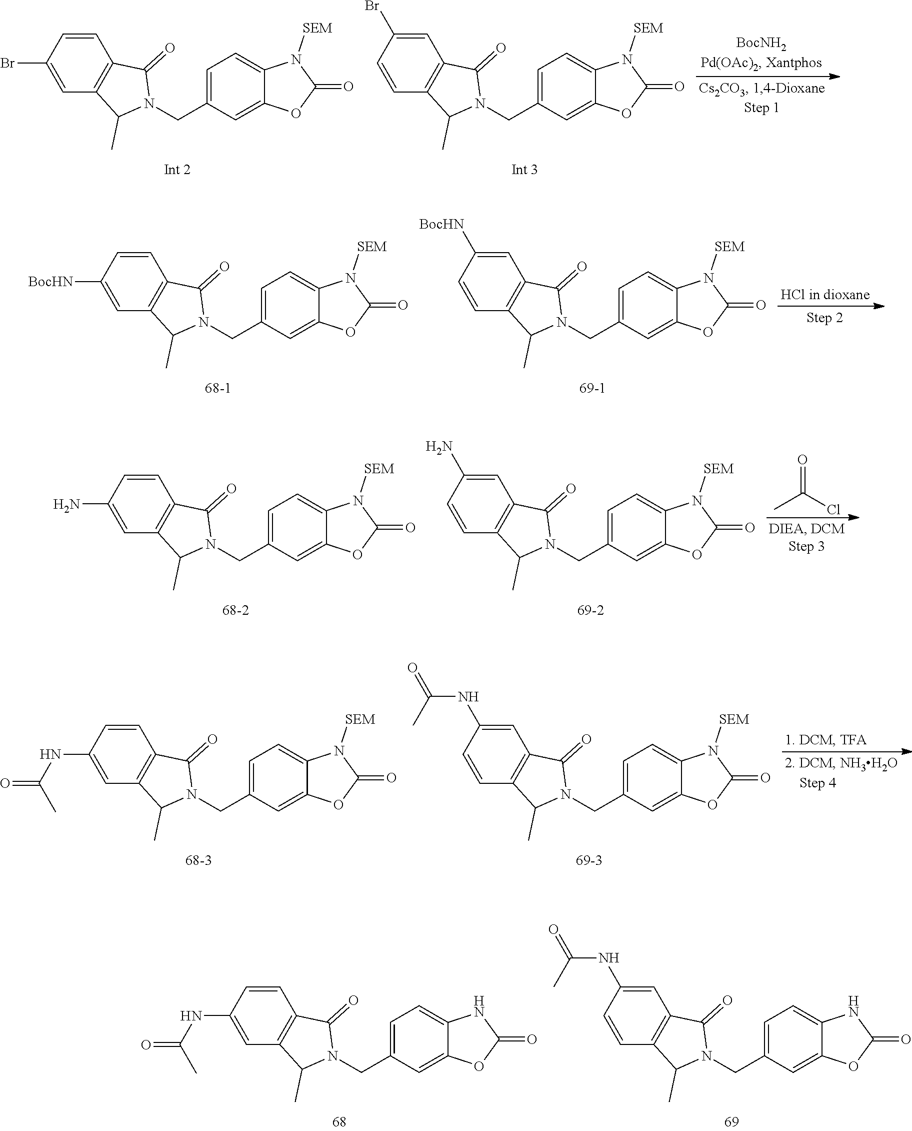

### Step 1. Mixture of tert-butyl (3-methyl-1-oxo-2-((2-oxo-3-((2-(trimethylsilyl) ethoxy)methyl)-2,3-dihydrobenzo[d]oxazol-6-yl)methyl)isoindolin-5-yl)carbamate (68-1) and tert-butyl(1-methyl-3-oxo-2-((2-oxo-3-((2-(trimethylsilyl)ethoxy)methyl)-2,3-dihydrobenzo[d]oxazol-6-yl)methyl)isoindolin-5-yl)carbamate (69-1)

To a solution of mixture of Int 2 and Int 3 (300 mg, 0.59 mmol) in 1,4-Dioxane (10 mL) were added tert-butyl carbamate (600 mg, 1.19 mmol), XantPHOS (69 mg, 0.12 mmol), Pd(OAc)₂ (27 mg, 0.12 mmol) and Cs₂CO₃ (777 mg, 2.38 mmol) under N₂. The mixture was purified by silica gel flash column chromatography (PE/EA=1.5/1) to give a mixture of tert-butyl (3-methyl-1-oxo-2-((2-oxo-3-((2-(trimethylsilyl)ethoxy)methyl)-2,3-dihydrobenzo[d]oxazol-6-yl)methyl) isoindolin-5-yl)carbamate and tert-butyl(1-methyl-3-oxo-2-((2-oxo-3-((2-(trimethylsilyl) ethoxy)methyl)-2,3-dihydrobenzo[d]oxazol-6-yl)methyl)isoindolin-5-yl)carbamate (500 mg, 77%) as a brown solid. MS (ESI) m/z 540 [M+H]⁺.

### Step 2. Mixture of 6-((5-amino-3-methyl-1-oxoisoindolin-2-yl)methyl)-3-((2-(trimethylsilyl)ethoxy)methyl)benzo[d]oxazol-2(3H)-one (68-2) and 6-((5-amino-1-methyl-3-oxoisoindolin-2-yl)methyl)-3-((2-(trimethylsilyl)ethoxy)methyl)benzo[d]oxazol-2(3H)-one (69-2)

To a solution of mixture of 68-1 and 69-1 (500 mg, 0.93 mmol) in DCM (3 mL), 4M HCl in 1,4-Dioxane (3 mL) was added, and stirred for 2 h, the mixture was concentrated under vacuum to give a mixture of 6-((5-amino-3-methyl-1-oxoisoindolin-2-yl)methyl)-3-((2-(trimethylsilyl) ethoxy)methyl)benzo[d]oxazol-2(3H)-one and 6-((5-amino-1-methyl-3-oxoisoindolin-2-yl)methyl)-3-((2-(trimethylsilyl)ethoxy)methyl)benzo[d]oxazol-2(3H)-one (400 mg, crude) as a brown solid, which was used in the next step without any further purification. MS (ESI) m/z 440 [M+H]⁺.

### Step 3. Mixture of N-(3-methyl-1-oxo-2-((2-oxo-3-((2-(trimethylsilyl)ethoxy) methyl)-2,3-dihydrobenzo[d]oxazol-6-yl)methyl)isoindolin-5-yl)acetamide (68-3) and N-(1-methyl-3-oxo-2-((2-oxo-3-((2-(trimethylsilyl)ethoxy)methyl)-2,3-dihydrobenzo[d]oxazol-6-yl)methyl)isoindolin-5-yl)acetamide (69-3)

To a solution of mixture of 68-2 and 69-2 (400 mg, 0.44 mmol) in DCM (4 mL) were added DIEA (0.32 mL, 1.82 mmol) and acetyl chloride (78 mg, 1 mmol) under N₂. The mixture was stirred for 2 h, and purified by prep TLC (DCM/MeOH=40/1) to give a mixture of N-(3-methyl-1-oxo-2-((2-oxo-3-((2-(trimethylsilyl)ethoxy)methyl)-2,3-dihydrobenzo[d]oxazol-6-yl)methyl) isoindolin-5-yl)acetamide and N-(1-methyl-3-oxo-2-((2-oxo-3-((2-(trimethylsilyl)ethoxy) methyl)-2,3-dihydrobenzo[d]oxazol-6-yl) methyl)isoindolin-5-yl)acetamide (240 mg, 54%) as a brown solid. MS (ESI) m/z 482 [M+H]⁺.

### Step 4. N-(3-methyl-1-oxo-2-((2-oxo-2,3-dihydrobenzo[d]oxazol-6-yl)methyl) isoindolin-5-yl)acetamide (68) and N-(1-methyl-3-oxo-2-((2-oxo-2,3-dihydrobenzo[d]oxazol-6-yl) methyl)isoindolin-5-yl)acetamide (69)

To a solution of mixture of 68-3 and 69-3 (240 mg, 0.50 mmol) in DCM (6 mL) was added TFA (2 mL). The mixture was stirred for 3 h and then concentrated under vacuum. The residue was taken in DCM (6 mL) and ammonium hydroxide (2 mL) was added at 0° C. This suspension was allowed to stir for an additional 1 h. The mixture was concentrated and purified by prep HPLC to afford two compounds: compound 68 and compound 69. One of these two compounds was a white solid (7 mg, 8%), with RT=2.69 min (HPLC), MS (ESI) m/z 352 [M+H]⁺. ¹H NMR (400 MHz, DMSO-d₆) δ 11.59 (s, 1H), 10.23 (s, 1H), 7.86 (s, 1H), 7.63 (d, J=8.3 Hz, 1H), 7.57 (dd, J=8.3, 1.8 Hz, 1H), 7.21 (s, 1H), 7.11-6.99 (m, 2H), 4.97 (d, J=15.2 Hz, 1H), 4.44-4.37 (m, 2H), 2.07 (s, 3H), 1.35 (d, J=6.7 Hz, 3H). The other one of the two compounds was a white solid, (6 mg, 6.9%), with RT=2.83 min (HPLC), MS (ESI) m/z 352 [M+H]⁺. ¹H NMR (400 MHz, DMSO-d₆) δ 11.64 (s, 1H), 10.15 (s, 1H), 8.05 (d, J=2.0 Hz, 1H), 7.66 (dd, J=8.2, 2.0 Hz, 1H), 7.48 (d, J=8.2 Hz, 1H), 7.21 (s, 1H), 7.07 (dd, J=8.0, 2.0 Hz, 1H), 7.02 (d, J=8.0 Hz, 1H), 4.99 (d, J=15.2 Hz, 1H), 4.43-4.33 (m, 2H), 2.07 (s, 3H), 1.36 (d, J=6.6 Hz, 3H).

### N-(3-methyl-1-oxo-2-((2-oxo-2,3-dihydrobenzo[d]oxazol-6-yl)methyl)isoindolin-5-yl) methanesulfonamide (70) and N-(1-methyl-3-oxo-2-((2-oxo-2,3-dihydrobenzo[d]oxazol-6-yl)methyl)isoindolin-5-yl)methanesulfonamide (71)

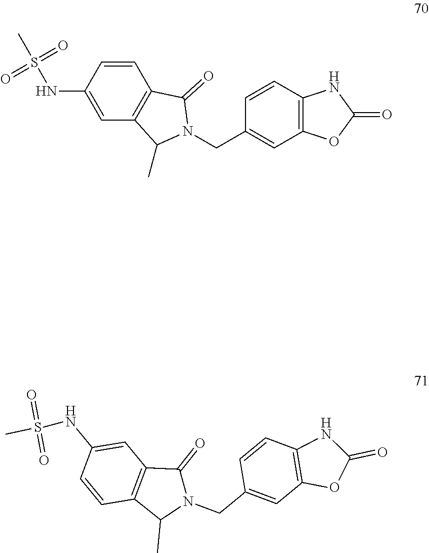 [SMILES: CC1c2cc(NS(C)(=O)=O)ccc2C(=O)N1Cc1ccc2[nH]c(=O)oc2c1 ; CC1c2ccc(NS(C)(=O)=O)cc2C(=O)N1Cc1ccc2[nH]c(=O)oc2c1]

The title compounds 70 and 71 were prepared according to the procedure described for compounds 68 and 69. After pre-HPLC separation, two compounds were obtained: compound 70 and compound 71. One of these two compounds was a white solid (3 mg, 5%), with RT=2.85 min (HPLC), MS (ESI) m/z 388 [M+H]⁺. ¹H NMR (400 MHz, DMSO-d₆) δ 11.06 (s, 1H), 8.37 (s, 1H), 7.62 (d, J=8.2 Hz, 1H), 7.28 (s, 1H), 7.26-7.17 (m, 2H), 7.09-6.99 (m, 2H), 4.97 (d, J=15.2 Hz, 1H), 4.42-4.33 (m, 2H), 3.01 (s, 3H), 1.36 (d, J=6.6 Hz, 3H). The other one of the two compounds was a white solid (1 mg, 2%), with RT=3.02 min (HPLC), MS (ESI) m/z 388 [M+H]⁺. ¹H NMR (400 MHz, DMSO-d₆) δ 10.76 (s, 1H), 8.42 (s, 1H), 7.55-7.49 (m, 2H), 7.39 (dd, J=8.2, 2.0 Hz, 1H), 7.18 (s, 1H), 7.08-6.97 (m, 2H), 4.99 (d, J=15.2 Hz, 1H), 4.41-4.36 (m, 2H), 2.99 (s, 3H), 1.37 (d, J=6.7 Hz, 3H).

### 6-((1-cyclopropyl-3-oxoisoindolin-2-yl)methyl)benzo[d]oxazol-2(3H)-one (72)

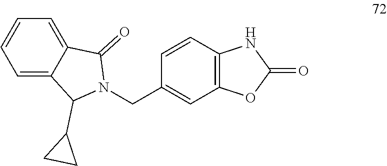 [SMILES: O=C1c2ccccc2C(C2CC2)N1Cc1ccc2[nH]c(=O)oc2c1]

The title compound 72 was prepared according to the procedure described for compound 67 as a white solid (16 mg, 19.2%). MS (ESI) m/z 321 [M+H]⁺. ¹H NMR (400 MHz, DMSO-d₆) δ 11.61 (s, 1H), 7.75 (d, J=7.5 Hz, 1H), 7.69-7.58 (m, 2H), 7.55-7.51 (m, 1H), 7.17 (s, 1H), 7.09-6.99 (m, 2H), 5.04 (d, J=15.4 Hz, 1H), 4.60 (d, J=15.4 Hz, 1H), 3.71 (d, J=8.0 Hz, 1H), 0.68-0.52 (m, 4H), 0.49-0.41 (m, 11H).

### (R)-6-((1-cyclopropyl-3-oxoisoindolin-2-yl)methyl)benzo[d]oxazol-2(3H)-one (73) and (S)-6-((1-cyclopropyl-3-oxoisoindolin-2-yl)methyl)benzo[d]oxazol-2(3H)-one (74)

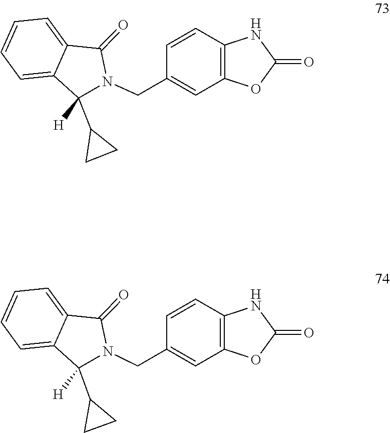 [SMILES: O=C1c2ccccc2[C@@H](C2CC2)N1Cc1ccc2[nH]c(=O)oc2c1 ; O=C1c2ccccc2[C@H](C2CC2)N1Cc1ccc2[nH]c(=O)oc2c1]

Chiral separation of compound 72 by chiral prep-HPLC (column: CHIRALPAK IG; column size: 2 cm×25 cm, 5 μm; mobile phase A: MTBE; mobile phase B: MeOH:DCM) was conducted to afford two enantiomers: compound 73 and 74. One of these two enantiomers was a white solid, with RT=4.563 min (CHIRAL HPLC, column: CHIRALPAK IG-3; column size: 4.6*50 mm, 3 μm; mobile phase: MTBE (0.1% DEA):(MeOH:DCM=1:1)=95:5; flow: 1.0 mL/min)−. MS (ESI) m/z 321 [M+H]⁺. The other one of the two enantiomers was a white solid, with RT=3.760 min (CHIRAL HPLC, column: CHIRALPAK IG-3; column size: 4.6*50 mm, 3 μm; mobile phase: MTBE (0.1% DEA):(MeOH:DCM=1:1)=95:5; flow: 1.0 mL/min): MS (ESI) m/z 321 [M+H]⁺.

### 6-((5-amino-3-methyl-1-oxoisoindolin-2-yl)methyl)benzo[d]oxazol-2(3H)-one (75) and 6-((5-amino-1-methyl-3-oxoisoindolin-2-yl)methyl)benzo[d]oxazol-2(3H)-one (76)

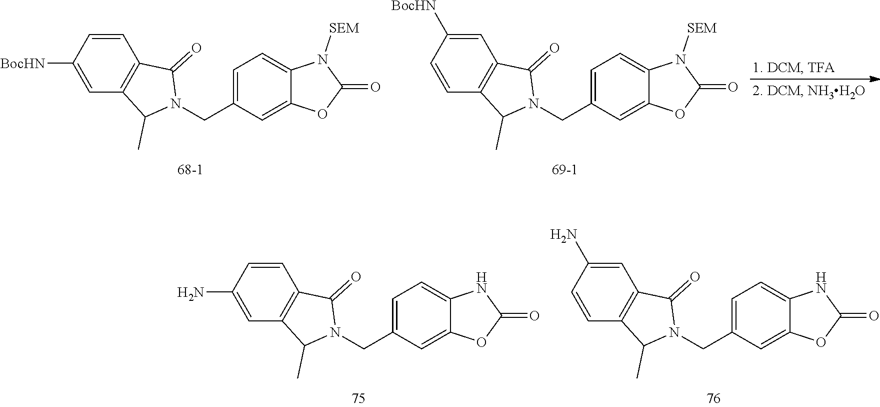

To a solution of mixture of 68-1 and 69-1 (200 mg, 0.37 mmol) in DCM (3 mL) was added TFA (1 mL). The mixture was stirred for 3 h and then concentrated under vacuum. The residue was taken in DCM (3 mL) and ammonium hydroxide (1 mL) was added at 0° C. This suspension was allowed to stir for an additional 1 h. The mixture was concentrated and purified by prep HPLC to give two compounds: compound 75 and compound 76. One of the two compounds was a white solid (12 mg, 21%), with RT=2.25 min (HPLC), MS (ESI) m/z 310 [M+H]⁺. ¹H NMR (400 MHz, DMSO-d₆) δ 11.61 (s, 1H), 7.38-7.30 (m, 1H), 7.21 (s, 1H), 7.10-6.99 (m, 4H), 4.97 (d, J=15.2 Hz, 1H), 4.38 (d, J=15.2 Hz, 1H), 4.32 (q, J=6.4 Hz, 1H), 1.33 (d, J=6.6 Hz, 3H). The other one of the two compounds was a white solid (26 mg, 45%), with RT=2.45 min (HPLC), MS (ESI) m/z 310 [M+H]⁺. ¹H NMR (400 MHz, DMSO-d₆) δ 11.57 (s, 1H), 7.34 (d, J=8.2 Hz, 1H), 7.18 (s, 1H), 7.09-6.99 (m, 2H), 6.61 (dd, J=8.0, 2.0 Hz, 1H), 6.57 (s, 1H), 4.92 (d, J=15.2 Hz, 1H), 4.31 (d, J=15.3 Hz, 1H), 4.22 (q, J=6.6 Hz, 1H), 1.30 (d, J=6.6 Hz, 3H).

### N-(3-methyl-1-oxo-2-((2-oxo-2,3-dihydrobenzo[d]oxazol-6-yl)methyl)isoindolin-5-yl) benzenesulfonamide (77) and N-(1-methyl-3-oxo-2-((2-oxo-2,3-dihydrobenzo[d]oxazol-6-yl)methyl)isoindolin-5-yl)benzenesulfonamide (78)

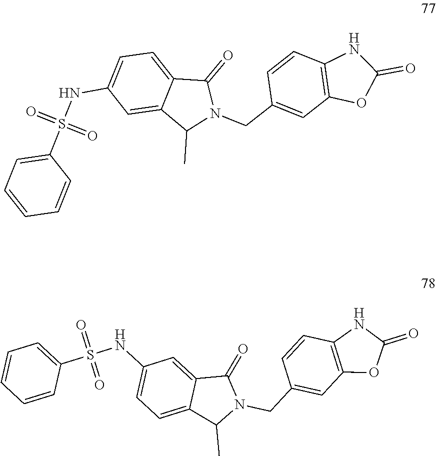 [SMILES: CC1c2cc(NS(=O)(=O)c3ccccc3)ccc2C(=O)N1Cc1ccc2[nH]c(=O)oc2c1 ; CC1c2ccc(NS(=O)(=O)c3ccccc3)cc2C(=O)N1Cc1ccc2[nH]c(=O)oc2c1]

The title compounds 77 and 78 were prepared according to the procedure described for compounds 68 and 69. After pre-HPLC separation, two compounds were obtained: compound 77 and compound 78. One of the two compounds was a white solid (26 mg, 51%), with RT=3.95 min (HPLC), MS (ESI) m/z 450 [M+H]H⁺. ¹H NMR (400 MHz, DMSO-d₆) b 10.96 (bs, 2H), 7.84-7.77 (m, 2H), 7.64-7.57 (m, 1H), 7.57-7.49 (m, 3H), 7.25 (d, J=1.9 Hz, 1H), 7.25 (d, J=2.0 Hz, 1H), 7.16 (dd, J=8.4, 2.0 Hz, 1H), 7.09-6.98 (m, 2H), 4.92 (d, J=15.2 Hz, 1H), 4.40-4.31 (m, 2H), 1.28 (d, J=6.7 Hz, 3H). The other one of the two compounds was a white solid (20 mg, 39%), with RT=4.10 min (HPLC), MS (ESI) m/z 450 [M+H]⁺. ¹H NMR (400 MHz, DMSO-d₆) δ 11.14 (bs, 2H), 7.82-7.74 (m, 2H), 7.65-7.51 (m, 3H), 7.42 (d, J=8.2 Hz, 1H), 7.38 (d, J=2.0 Hz, 11H), 7.29 (dd, J=8.2, 2.1 Hz, 1H), 7.20 (d, J=1.6 Hz, 11H), 7.11-6.98 (m, 2H), 4.93 (d, J=15.2 Hz, 1H), 4.40-4.29 (m, 2H), 1.30 (d, J=6.7 Hz, 3H).

### 6-((5-((2-azaspiro[3.3]heptan-6-yl)amino)-3-methyl-1-oxoisoindolin-2-yl)methyl)benzo[d]oxazol-2(3H)-one (79)

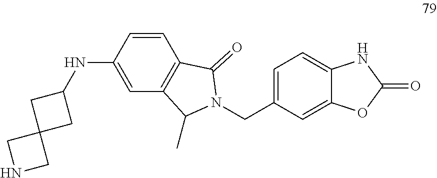 [SMILES: CC1c2cc(NC3CC4(CNC4)C3)ccc2C(=O)N1Cc1ccc2[nH]c(=O)oc2c1]

The title compound 79 was prepared according to the procedure described for compound 47 using tert-butyl 6-amino-2-azaspiro[3.3]heptane-2-carboxylate as a starting material to afford a white solid (9 mg, 21%). MS (ESI) m/z 405 [M+H]⁺. ¹H NMR (400 MHz, DMSO-d₆) δ 9.53 (bs, 2H), 7.39 (d, J=8.3 Hz, 11H), 7.18 (s, 1H), 7.09-6.99 (m, 2H), 6.60-6.52 (m, 2H), 6.49 (s, 11H), 4.93 (d, J=15.3 Hz, 1H), 4.32 (d, J=15.3 Hz, 11H), 4.24 (q, J=6.6 Hz, 11H), 4.02 (s, 2H), 3.92 (s, 2H), 3.80-3.71 (m, 1H), 2.71-2.64 (m, 2H), 2.08-1.97 (m, 2H), 1.32 (d, J=6.6 Hz, 3H).

### Mixture of (E)-6-((1-benzylidene-3-oxoisoindolin-2-yl)methyl)benzo[d]oxazol-2(3H)-one (80-1) and (Z)-6-((1-benzylidene-3-oxoisoindolin-2-yl)methyl)benzo[d]oxazol-2(3H)-one (80-2)

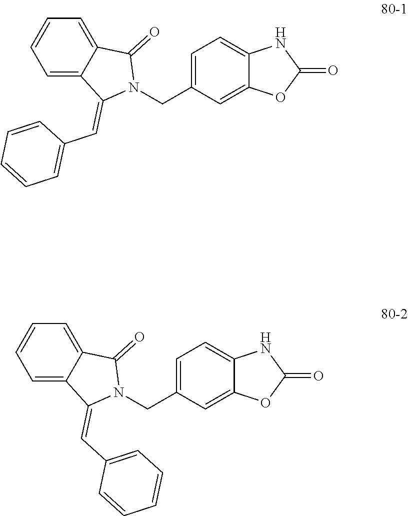 [SMILES: O=C1c2ccccc2/C(=C\c2ccccc2)N1Cc1ccc2[nH]c(=O)oc2c1 ; O=C1c2ccccc2/C(=C/c2ccccc2)N1Cc1ccc2[nH]c(=O)oc2c1]

The mixture of compounds 80-1 and 80-2 were prepared according to general procedure F as a white solid (42 mg, 33%). MS (ESI) m/z 369 [M+H]⁺. ¹H NMR (400 MHz, DMSO-d₆) δ 11.60 (s, 1H), 7.83 (d, J=7.6 Hz, 1H), 7.57-7.53 (m, 1H), 7.49-7.36 (m, 6H), 7.31-7.28 (m, 2H), 7.13 (dd, J=8.0, 1.6 Hz, 11H), 7.06 (d, J=8.0 Hz, 1H), 6.78 (s, 1H), 5.11 (s, 2H). A mixture of intermediate 80-4 and 80-5 was prepared as follows:

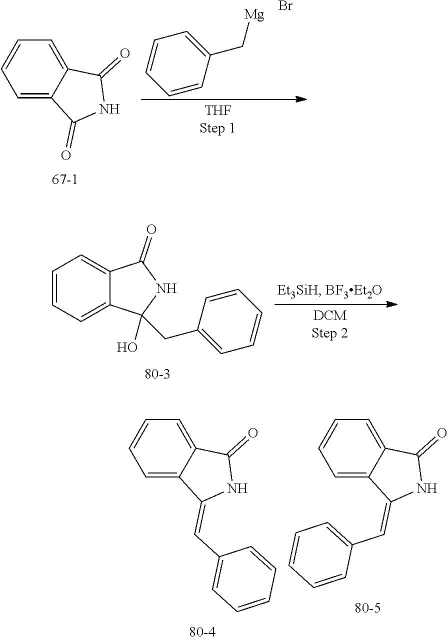

### Step 1. 3-benzyl-3-hydroxyisoindolin-1-one (80-3)

To a solution of isoindoline-1,3-dione (2 g, 13.6 mmol) in THF (20 mL) was added benzylmagnesium bromide (40.8 mL, 40.8 mmol) at 0′C. The resulting mixture was stirred for 3 h at rt and diluted with EA (30 mL) and washed with water (3×20 mL). The organic layer was separated, dried over anhydrous Na₂SO₄ and concentrated under vacuum to afford the title compound (3.25 g, crude) as a yellow solid. MS (ESI) m/z 240 [M+H]⁺.

### Step 2. Mixture of (Z)-3-benzylideneisoindolin-1-one (80-4) and (E)-3-benzylideneisoindolin-1-one (80-5)

To a solution of 3-benzyl-3-hydroxyisoindolin-1-one (3.25 g, 13.6 mmol) in DCM (10 mL) were added triethylsilane (7.9 g, 68 mmol) and borontrifluoridediethyletherate (3.9 g, 27.2 mmol) at r.t. The resulting mixture was stirred for 3 h at rt and diluted with EA (30 mL) and washed with water (3×20 mL). The organic layer was separated, dried over anhydrous Na₂SO₄ and concentrated under vacuum. The residue was purified by TLC (PE/EA=1/3) to afford the title compound (1.5 g, 49.9%) as a yellow solid. MS (ESI) m/z 222 [M+H]⁺.

### 6-((5-(cyclopentylamino)-3-methyl-1-oxoisoindolin-2-yl)methyl)benzo[d]oxazol-2(3H)-one (81)

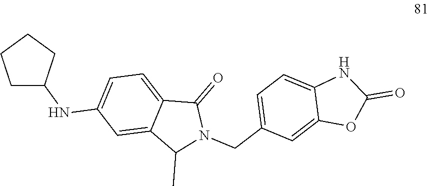 [SMILES: CC1c2cc(NC3CCCC3)ccc2C(=O)N1Cc1ccc2[nH]c(=O)oc2c1]

The title compound 81 was prepared according to the procedure described for compound 47 as a white solid (3 mg, 5%). MS (ESI) m/z 378 [M+H]⁺. ¹H NMR (400 MHz, DMSO-d₆) δ 11.58 (s, 1H), 7.36 (d, J=8.3 Hz, 1H), 7.17 (s, 1H), 7.07-6.98 (m, 2H), 6.62 (dd, J=8.3, 2.0 Hz, 1H), 6.57 (d, J=2.0 Hz, 1H), 6.29 (d, J=6.5 Hz, 1H), 4.93 (d, J=15.2 Hz, 1H), 4.30 (d, J=15.2 Hz, 1H), 4.22 (q, J=6.6 Hz, 1H), 3.79-3.68 (m, 1H), 1.99-1.86 (m, 2H), 1.71-1.63 (m, 2H), 1.67-1.49 (m, 2H), 1.49-1.39 (m, 2H), 1.32 (d, J=6.7 Hz, 3H).

### N-(4-((1-methyl-3-oxoisoindolin-2-yl)methyl)phenyl)acetamide (82)

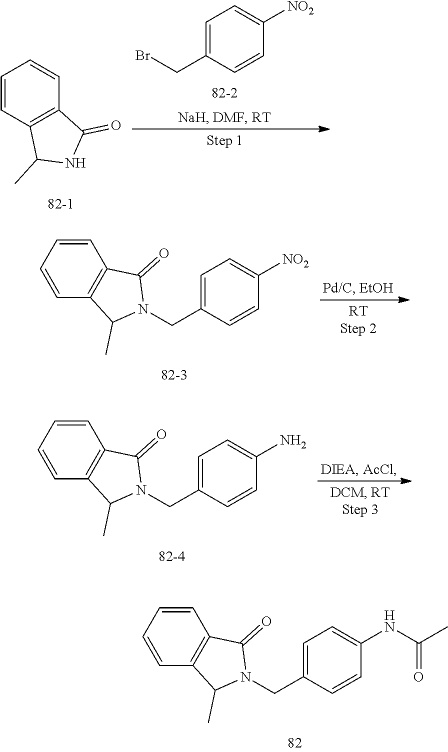 [SMILES: CC1NC(=O)c2ccccc21 ; O=[N+]([O-])c1ccc(CBr)cc1 ; CC1c2ccccc2C(=O)N1Cc1ccc([N+](=O)[O-])cc1 ; CC1c2ccccc2C(=O)N1Cc1ccc(N)cc1 ; CC(=O)Nc1ccc(CN2C(=O)c3ccccc3C2C)cc1]

### Step 1. 3-methyl-2-(4-nitrobenzyl)isoindolin-1-one (82-3)

To a solution of 3-methylisoindolin-1-one (500 mg, 3.4 mmol) in DMF (20 mL) was added NaH (150 mg, 60% in mineral oil, 3.7 mmol) at 0° C. under N₂. The mixture was stirred for 10 min at 0° C. 1-(bromomethyl)-4-nitrobenzene (808 mg, 3.7 mmol) was added at 0° C. The mixture was stirred for 4 h at r.t. Water (50 mL) was added solution, the resulting mixture was extracted with EtOAc (50 mL*3), the combined organic layers were washed with water (50 mL*2) and brine (50 mL), dried over anhydrous Na₂SO₄, filtered and concentrated under vacuum. The residue was purified by silica gel column chromatography (PE/EtOAc=1/1) to afford 3-methyl-2-(4-nitrobenzyl)isoindolin-1-one (210 mg, 38%) as a yellow solid. MS (ESI) m/z 283 [M+H]⁺.

### Step 2. 2-(4-aminobenzyl)-3-methylisoindolin-1-one (82-4)

To a solution of 3-methyl-2-(4-nitrobenzyl)isoindolin-1-one (210 mg, 0.7 mmol) in EtOH (20 mL) was added Pd/C (30 mg) at r.t. The mixture was stirred at r.t. for 4 h under hydrogen balloon. The reaction was filtered, and the filtrate was concentrated. The residue was purified by silica gel flash column chromatography (PE/EtOAc=1/2) to afford 2-(4-aminobenzyl)-3-methylisoindolin-1-one (130 mg, 69%) as a yellow solid. MS (ESI) m/z 253 [M+H]⁺.

### Step 3. N-(4-((1-methyl-3-oxoisoindolin-2-yl)methyl)phenyl)acetamide (82)

To a solution of 2-(4-aminobenzyl)-3-methylisoindolin-1-one (40 mg, 0.2 mmol) in DCM (5 mL) were added DIEA (41 mg, 0.3 mmol) and AcCl (12 mg, 0.2 mmol) at ice-bath. The mixture was stirred for 2 h at r.t. Water (5 mL) was added, and the mixture was extracted with DCM (10 mL*2). The combined organic layers were washed with brine (10 mL), dried over anhydrous Na₂SO₄, filtered and concentrated under vacuum. The residue was purified by prep-HPLC to give N-(4-((1-methyl-3-oxoisoindolin-2-yl)methyl)phenyl)acetamide (34 mg, 72%) as a white solid. MS (ESI) m/z 295 [M+H]⁺. ¹H NMR (400 MHz, DMSO-d₆) δ 9.91 (s, 1H), 7.71 (d, J=7.6 Hz, 1H), 7.60-7.57 (m, 2H), 7.53-7.47 (m, 3H), 7.21 (d, J=8.4 Hz, 2H), 4.96 (d, J=15.2 Hz, 1H), 4.41 (q, J=6.4 Hz, 11H), 4.34 (d, J=15.6 Hz, 11H), 2.01 (s, 3H), 1.37 (d, J=6.8 Hz, 3H).

### Mixture of 6-((3-methyl-1-oxo-5-(pyridin-3-yl)isoindolin-2-yl)methyl)benzo[d]oxazol-2(3H)-one (83) and 6-((1-methyl-3-oxo-5-(pyridin-3-yl)isoindolin-2-yl)methyl)benzo[d]oxazol-2(3H)-one (84)

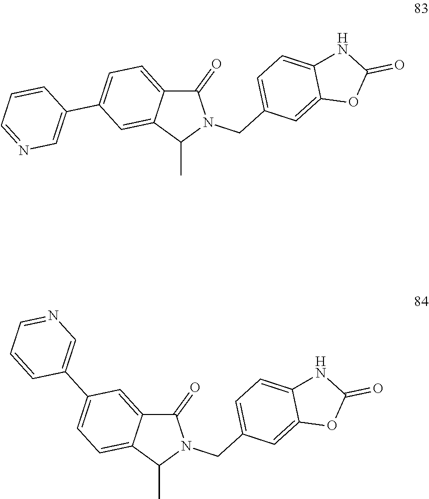 [SMILES: CC1c2cc(-c3cccnc3)ccc2C(=O)N1Cc1ccc2[nH]c(=O)oc2c1 ; CC1c2ccc(-c3cccnc3)cc2C(=O)N1Cc1ccc2[nH]c(=O)oc2c1]

A mixture of compounds 83 and 84 was prepared according to the procedure described for compounds 50 and 51 as a white solid (17 mg, 11.5%). MS (ESI) m/z 372 [M+H]⁺. ¹H NMR (400 MHz, DMSO-d₆) δ 11.60 (s, 1H), 8.95 (d, J=2.3 Hz, 1H), 8.63-8.59 (m, 1H), 8.18-8.09 (m, 1H), 8.02-7.94 (m, 1H), 7.88-7.68 (m, 2H), 7.54-7.48 (m, 1H), 7.27 (d, J=1.6 Hz, 1H), 7.14-7.01 (m, 2H), 5.05 (d, J=14.9 Hz, 1H), 4.55-4.40 (m, 2H), 1.47 (d, J=6.8 Hz, 1.5H), 1.44 (d, J=6.8 Hz, 1.5H).

### Mixture of 6-((5-(cyclopent-1-en-1-yl)-3-methyl-1-oxoisoindolin-2-yl)methyl)benzo[d]oxazol-2(3H)-one (85) and 6-((5-(cyclopent-1-en-1-yl)-1-methyl-3-oxoisoindolin-2-yl) methyl)benzo[d]oxazol-2(3H)-one (86)

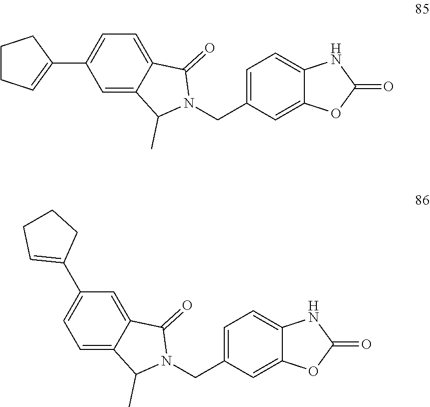 [SMILES: CC1c2cc(C3=CCCC3)ccc2C(=O)N1Cc1ccc2[nH]c(=O)oc2c1 ; CC1c2ccc(C3=CCCC3)cc2C(=O)N1Cc1ccc2[nH]c(=O)oc2c1]

A mixture of compounds 85 and 86 was prepared according to the procedure described for compounds 50 and 51 as a white solid (19 mg, 9.8%). MS (ESI) m/z 361 [M+H]⁺. ¹H NMR (400 MHz, DMSO-d₆) δ 11.61 (s, 1H), 7.85-7.46 (m, 3H), 7.23 (d, J=1.5 Hz, 1H), 7.14-6.97 (m, 2H), 6.46-6.37 (m, 1H), 5.01 (d, J=15.2 Hz, 0.5H), 4.99 (d, J=15.2 Hz, 0.5H), 4.50-4.34 (m, 2H), 2.77-2.63 (m, 2H), 2.55-2.52 (m, 2H), 2.04-1.90 (m, 2H), 1.42-1.37 (m, 3H).

### Mixture of 6-((5-cyclopentyl-3-methyl-1-oxoisoindolin-2-yl)methyl)benzo[d]oxazol-2(3H)-one (87) and 6-((5-cyclopentyl-1-methyl-3-oxoisoindolin-2-yl)methyl)benzo[d]oxazol-2(3H)-one (88)

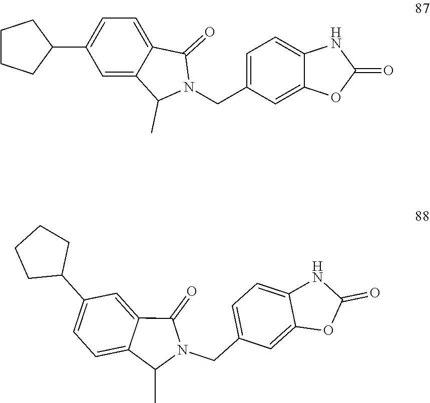 [SMILES: CC1c2cc(C3CCCC3)ccc2C(=O)N1Cc1ccc2[nH]c(=O)oc2c1 ; CC1c2ccc(C3CCCC3)cc2C(=O)N1Cc1ccc2[nH]c(=O)oc2c1]

A mixture of compounds 87 and 88 was prepared according to the procedure described for compounds 50 and 51 as a white solid (14 mg, 9.6%). MS (ESI) m/z 363 [M+H]⁺. ¹H NMR (400 MHz, DMSO-d) δ 11.60 (s, 1H), 7.64-7.54 (m, 1H), 7.51-7.35 (m, 2H), 7.23-7.20 (m, 1H), 7.09-6.98 (m, 2H), 5.01 (d, J=15.2 Hz, 0.5H), 5.00 (d, J=15.2 Hz, 0.5H), 4.46-4.27 (m, 2H), 3.14-2.98 (m, 11H), 2.10-1.97 (m, 2H), 1.85-1.49 (m, 6H), 1.40-1.35 (m, 3H).

### 4-fluoro-6-((1-methyl-3-oxoisoindolin-2-yl)methyl)benzo[d]oxazol-2(3H)-one (89)

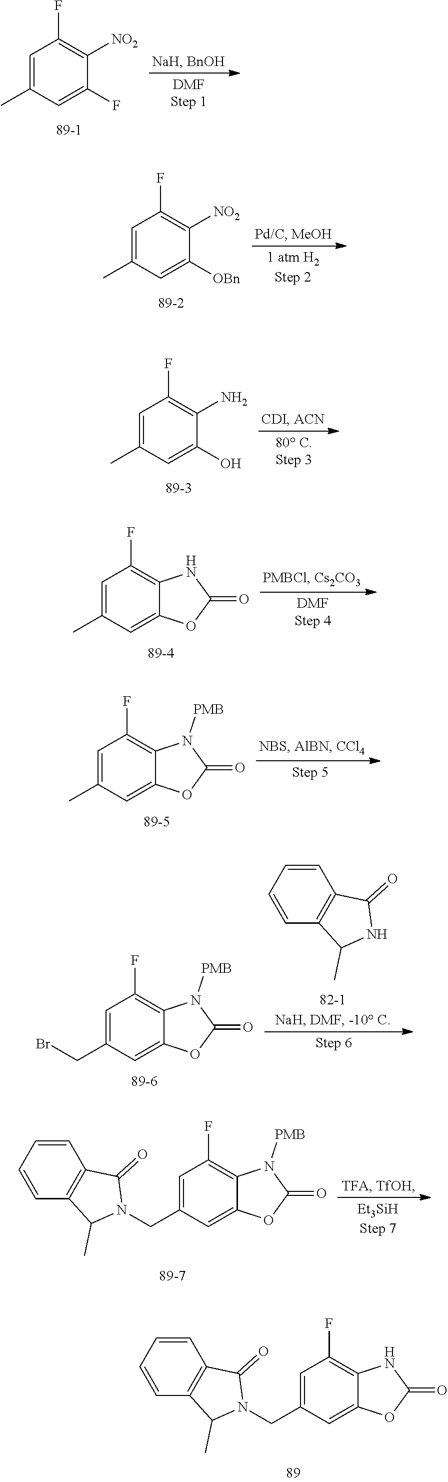

### Step 1. 1-(benzyloxy)-3-fluoro-5-methyl-2-nitrobenzene (89-2)

To a solution of BnOH (2.7 g, 25 mmol) in DMF (100 mL) was added NaH (1 g, 60% in mineral oil, 25 mmol) at 0° C. The mixture was stirred for 30 min at 0° C. Then 1,3-difluoro-5-methyl-2-nitrobenzene (4 g, 23.1 mmol) was added at 0° C. The reaction was stirred for 1 h at r.t. H₂O (50 mL) was added slowly, and the resulting mixture was extracted with EA (50 mL*2). The combined organic layers were washed with H₂O (100 mL*2) and brine (50 mL), dried over Na₂SO₄, filtered and concentrated. The residue was purified by FCC (PE/EA=10/1) to give 1-(benzyloxy)-3-fluoro-5-methyl-2-nitrobenzene (2.3 g, 38%) as a yellow solid. MS (ESI) m/z 262 [M+H]⁺.

### Step 2. 2-amino-3-fluoro-5-methylphenol (89-3)

A mixture of 1-(benzyloxy)-3-fluoro-5-methyl-2-nitrobenzene (2.3 g, 8.8 mmol) and Pd/C (690 mg, 5%) in MeOH (40 mL) was stirred at rt overnight under 1 atm H₂. The mixture was filtered, concentrated. Then the residue was washed with PE (5 mL) to give 2-amino-3-fluoro-5-methylphenol (1.1 g, 89%) as a brown solid. MS (ESI) m/z 141 [M+H]⁺.

### Step 3. 4-fluoro-6-methylbenzo[d]oxazol-2(3H)-one (89-4)

A mixture of 2-amino-3-fluoro-5-methylphenol (1.2 g, 7.1 mmol), CDI (2.3 g, 14.2 mmol) in ACN (35 mL) was stirred at 80° C. overnight. The mixture was poured into ice water (50 mL), extracted with EA (50 mL*2). The combined organic layers were washed with H₂O (100 mL*2) and brine (50 mL), dried over Na₂SO₄. The residue was purified by trituration with PE to give 4-fluoro-6-methylbenzo[d]oxazol-2(3H)-one (1.1 g, 92%) as a white solid. MS (ESI) m/z 168 [M+H]⁺.

### Step 4. 4-fluoro-3-(4-methoxybenzyl)-6-methylbenzo[d]oxazol-2(3H)-one (89-5)

To a suspension of 4-fluoro-6-methylbenzo[d]oxazol-2(3H)-one (1.1 g, 6.5 mmol) and Cs₂CO₃ (4.3 g, 13.2 mmol) in DMF (50 mL) was added PMBCl (1.1 g, 6.5 mmol) at r.t. The reaction was stirred at 60° C. overnight. The mixture was poured into ice water (100 mL), extracted with EA (100 mL*2). The combined organic layers were washed with H₂O (100 mL*2) and brine (100 mL), dried over Na₂SO₄. Then the residue was purified by FCC (PE:EA=5:1) to give 4-fluoro-3-(4-methoxybenzyl)-6-methylbenzo[d]oxazol-2(3H)-one (1.4 g, 73%) as a white solid. MS (ESI) m/z 288 [M+H]⁺.

### Step 5. 6-(bromomethyl)-4-fluoro-3-(4-methoxybenzyl)benzo[d]oxazol-2(3H)-one (89-6)

A solution of 4-fluoro-3-(4-methoxybenzyl)-6-methylbenzo[d]oxazol-2(3H)-one (200 mg, 0.35 mmol), NBS (150 mg, 0.84 mmol), AIBN (12 mg, 0.07 mmol) in CCl₄ (5 mL) was stirred at 80° C. overnight. The mixture was concentrated and purified by FCC (PE:EA=5:1) to give 6-(bromomethyl)-4-fluoro-3-(4-methoxybenzyl)benzo[d]oxazol-2(3H)-one (80 mg, 32%) as a yellow solid. MS (ESI) m/z 366 [M+H]⁺.

### Step 6. 4-fluoro-3-(4-methoxybenzyl)-6-((1-methyl-3-oxoisoindolin-2-yl)methyl) benzo[d]oxazol-2(3H)-one (89-7)

To a solution of 3-methylisoindolin-1-one (32 mg, 0.22 mmol) in DMF (100 mL) was added NaH (10 mg, 60% in mineral oil, 0.23 mmol) at 0° C. The reaction was stirred for 30 min at 0° C. Then 6-(bromomethyl)-4-fluoro-3-(4-methoxybenzyl)benzo[d]oxazol-2(3H)-one (80 mg, 0.22 mmol) was added at 0° C. The reaction was stirred for 1 h at r.t. H₂O (50 mL) was added, the mixture was extracted with EA (50 mL*2). The combined organic layers were washed with H₂O (100 mL*2) and brine (50 mL), dried over Na₂SO₄, filtered and concentrated. The residue was purified by (PE/EA=5/1) to give 4-fluoro-3-(4-methoxybenzyl)-6-((1-methyl-3-oxoisoindolin-2-yl)methyl)benzo[d]oxazol-2(3H)-one (38 mg, 40%) as a white solid. MS (ESI) m/z 433 [M+H]⁺.

### Step 7. 4-fluoro-6-((1-methyl-3-oxoisoindolin-2-yl)methyl)benzo[d]oxazol-2(3H)-one (89)

TfOH (0.23 mL) was added to a stirred solution of 4-fluoro-3-(4-methoxybenzyl)-6-((1-methyl-3-oxoisoindolin-2-yl)methyl)benzo[d]oxazol-2(3H)-one (38 mg, 0.30 mmol) and SiHEt₃ (19 mg, 0.17 mmol) in TFA (1.2 mL) at r.t. The mixture was stirred at r.t. for 2 h. The mixture was diluted with ice water (10 mL). Then Na₂CO₃ (aq.) was added until pH 9. The mixture was extracted with DCM (10 mL*2). The combined organic layers were dried over Na₂SO₄, filtered and concentrated in vacuo. The residue was purified by prep-HPLC to give 4-fluoro-6-((1-methyl-3-oxoisoindolin-2-yl)methyl)benzo[d]oxazol-2(3H)-one (16 mg, 58%) as a white solid. MS (ESI) m/z 313 [M+H]⁺. ¹H NMR (400 MHz, DMSO-d₆) δ 7.72 (d, J=7.2 Hz, 1H), 7.63-7.57 (m, 2H), 7.50 (td, J=7.6, 1.6 Hz, 1H), 7.06 (s, 1H), 7.00 (d, J=10.8 Hz, 1H), 4.98 (d, J=15.6 Hz, 1H), 4.48 (q, J=6.8 Hz, 1H), 4.42 (d, J=15.2 Hz, 1H), 1.40 (d, J=6.8 Hz, 3H).

### 3-methyl-1-oxo-2-((2-oxo-2,3-dihydrobenzo[d]oxazol-6-yl)methyl)isoindoline-5-carboxamide (90) and 1-methyl-3-oxo-2-((2-oxo-2,3-dihydrobenzo[d]oxazol-6-yl)methyl)isoindoline-5-carboxamide (91)

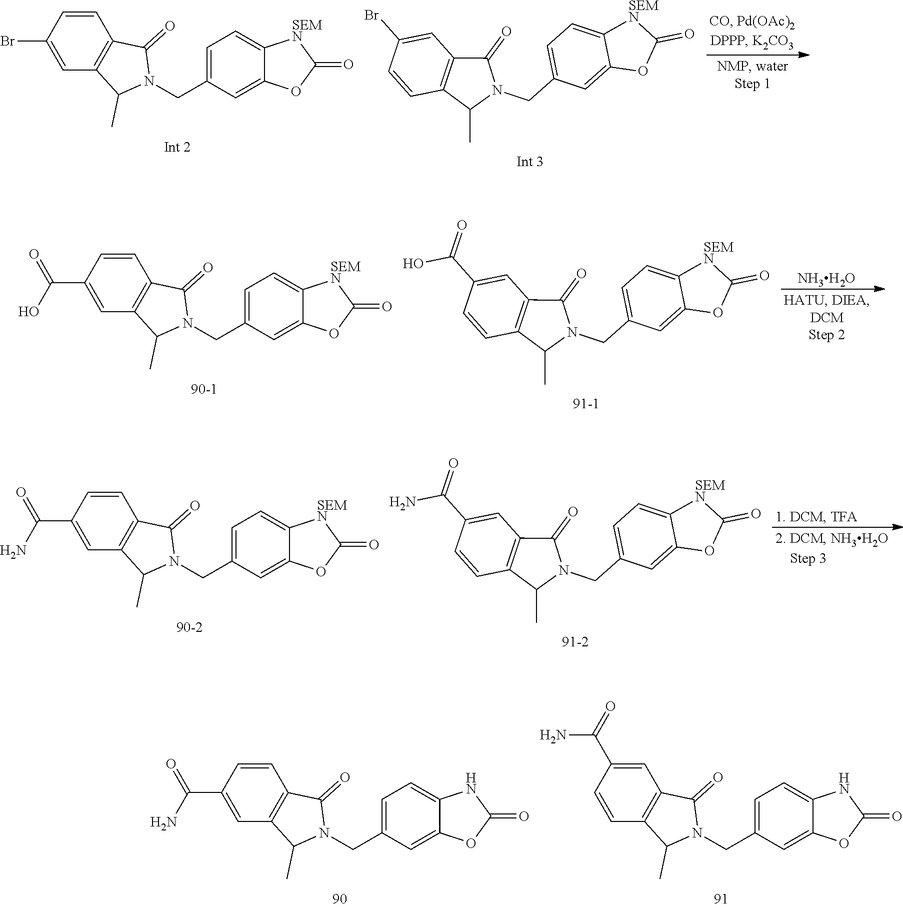

### Step 1. Mixture of 3-methyl-1-oxo-2-((2-oxo-3-((2-(trimethylsilyl)ethoxy)methyl)-2,3-dihydrobenzo[d]oxazol-6-yl)methyl)isoindoline-5-carboxylic acid (90-1) and 1-methyl-3-oxo-2-((2-oxo-3-((2-(trimethylsilyl)ethoxy)methyl)-2,3-dihydrobenzo[d]oxazol-6-yl) methyl)isoindoline-5-carboxylic acid (91-1)

To a solution of mixture of Int 2 and Int 3 (750 mg, 1.49 mmol) in NMP (15 mL) were added Pd(OAc)₂ (33 mg, 0.14 mmol), DPPP (61 mg, 0.14 mmol), K₂CO₃ (617 mg, 4.47 mmol) and Water (1.5 mL) under N₂ and then evacuated and placed under CO (4 atm). The mixture was stirred at 100° C. for 14 h. The mixture was purified by C18 column to give a mixture of 3-methyl-1-oxo-2-((2-oxo-3-((2-(trimethylsilyl)ethoxy)methyl)-2,3-dihydrobenzo[d]oxazol-6-yl)methyl) isoindoline-5-carboxylic acid and 1-methyl-3-oxo-2-((2-oxo-3-((2-(trimethylsilyl)ethoxy) methyl)-2,3-dihydrobenzo[d]oxazol-6-yl)methyl)isoindoline-5-carboxylic acid (500 mg, 71%) as a brown solid. MS (ESI) m/z 469 [M+H]⁺.

### Step 2. Mixture of 3-methyl-1-oxo-2-((2-oxo-3-((2-(trimethylsilyl)ethoxy)methyl)-2,3-dihydrobenzo[d]oxazol-6-yl)methyl)isoindoline-5-carboxamide (90-2) and 1-methyl-3-oxo-2-((2-oxo-3-((2-(trimethylsilyl)ethoxy)methyl)-2,3-dihydrobenzo[d]oxazol-6-yl)methyl) isoindoline-5-carboxamide (91-2)

To a solution of mixture of 90-1 and 91-1 (200 mg, 0.42 mmol) in DMF (4 mL) was added HATU (486 mg, 1.28 mmol), ammonium hydroxide (1 mL, 25.96 mmol) and DIEA (0.30 mL, 1.70 mmol) under N₂. The mixture was stirred for 1 h, diluted with EtOAc, and washed with water. The organic layer was separated, dried over anhydrous Na₂SO₄ and concentrated under vacuum. The residue was purified by prep TLC (DCM/MeOH=30/1) to give a mixture of 3-methyl-1-oxo-2-((2-oxo-3-((2-(trimethylsilyl)ethoxy)methyl)-2,3-dihydrobenzo[d]oxazol-6-yl)methyl)isoindoline-5-carboxamide and 1-methyl-3-oxo-2-((2-oxo-3-((2-(trimethylsilyl) ethoxy)methyl)-2,3-dihydrobenzo[d]oxazol-6-yl)methyl)isoindoline-5-carboxamide (110 mg, 55%) as a brown solid. MS (ESI) m/z 468 [M+H]⁺.

### Step 3. 3-methyl-1-oxo-2-((2-oxo-2,3-dihydrobenzo[d]oxazol-6-yl)methyl)isoindoline-5-carboxamide (90) and 1-methyl-3-oxo-2-((2-oxo-2,3-dihydrobenzo[d]oxazol-6-yl)methyl)isoindoline-5-carboxamide (91)

To a solution of mixture of 90-2 and 91-2 (110 mg, 0.22 mmol) in DCM (6 mL) was added TFA (2 mL). The mixture was stirred for 3 h and then concentrated under vacuum. The residue was taken in DCM (6 mL) and ammonium hydroxide (2 mL) was added at 0° C. This suspension was allowed to stir for an additional 1 h. The mixture was concentrated and purified by prep HPLC to afford two compounds: compound 90 and compound 91. One of the two compounds was a white solid (11 mg, 27.7%), with RT=2.29 min (HPLC), MS (ESI) m/z 338 [M+H]⁺. ¹H NMR (400 MHz, DMSO-d₆) δ 11.65 (s, 1H), 8.09 (s, 1H), 8.06 (s, 1H), 7.98 (dd, J=7.9, 1.7 Hz, 1H), 7.77 (d, J=7.9 Hz, 1H), 7.51 (s, 1H), 7.25 (d, J=1.5 Hz, 1H), 7.10 (dd, J=8.0, 1.6 Hz, 1H), 7.04 (d, J=7.9 Hz, 1H), 5.02 (d, J=15.1 Hz, 1H), 4.50 (q, J=6.7 Hz, 1H), 4.43 (d, J=15.2 Hz, 1H), 1.41 (d, J=6.7 Hz, 3H). The other one of the two compounds was a white solid (9 mg, 22.7%), with RT=2.45 min (HPLC), MS (ESI) m/z 338 [M+H]⁺. ¹H NMR (400 MHz, DMSO-d₆) δ 11.62 (s, 1H), 8.25 (d, J=1.6 Hz, 1H), 8.15 (s, 1H), 8.11 (dd, J=7.9, 1.7 Hz, 1H), 7.66 (d, J=7.9 Hz, 1H), 7.46 (s, 1H), 7.24 (d, J=1.5 Hz, 1H), 7.09 (dd, J=8.0, 1.6 Hz, 1H), 7.03 (d, J=7.9 Hz, 1H), 5.02 (d, J=15.1 Hz, 1H), 4.50 (q, J=6.7 Hz, 1H), 4.43 (d, J=15.2 Hz, 11H), 1.41 (d, J=6.7 Hz, 3H).

### N,3-dimethyl-1-oxo-2-((2-oxo-2,3-dihydrobenzo[d]oxazol-6-yl)methyl)isoindoline-5-carboxamide (92)

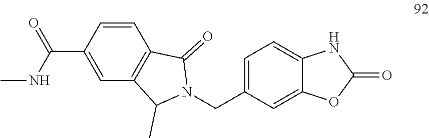 [SMILES: CNC(=O)c1ccc2c(c1)C(C)N(Cc1ccc3[nH]c(=O)oc3c1)C2=O]

The title compound 92 was prepared according to the procedure described for compounds 90 and 91 as a white solid (21 mg, 42%). MS (ESI) m/z 352 [M+H]⁺. ¹H NMR (400 MHz, DMSO-d₆) δ 11.62 (s, 1H), 8.57 (q, J=4.5 Hz, 1H), 8.01 (d, J=1.3 Hz, 1H), 7.93 (dd, J=7.9, 1.4 Hz, 1H), 7.78 (d, J=7.9 Hz, 1H), 7.24 (d, J=1.4 Hz, 1H), 7.09 (dd, J=8.0, 1.6 Hz, 1H), 7.03 (d, J=7.9 Hz, 1H), 5.01 (d, J=15.2 Hz, 1H), 4.51 (q, J=6.7 Hz, 1H), 4.44 (d, J=15.2 Hz, 1H), 2.80 (d, J=4.5 Hz, 3H), 1.42 (d, J=6.7 Hz, 3H).

### 3-(1-methyl-3-oxo-2-((2-oxo-2,3-dihydrobenzo[d]oxazol-6-yl)methyl)isoindolin-5-yl) propenamide (93) and 3-(3-methyl-1-oxo-2-((2-oxo-2,3-dihydrobenzo[d]oxazol-6-yl)methyl)isoindolin-5-yl)propenamide (94)

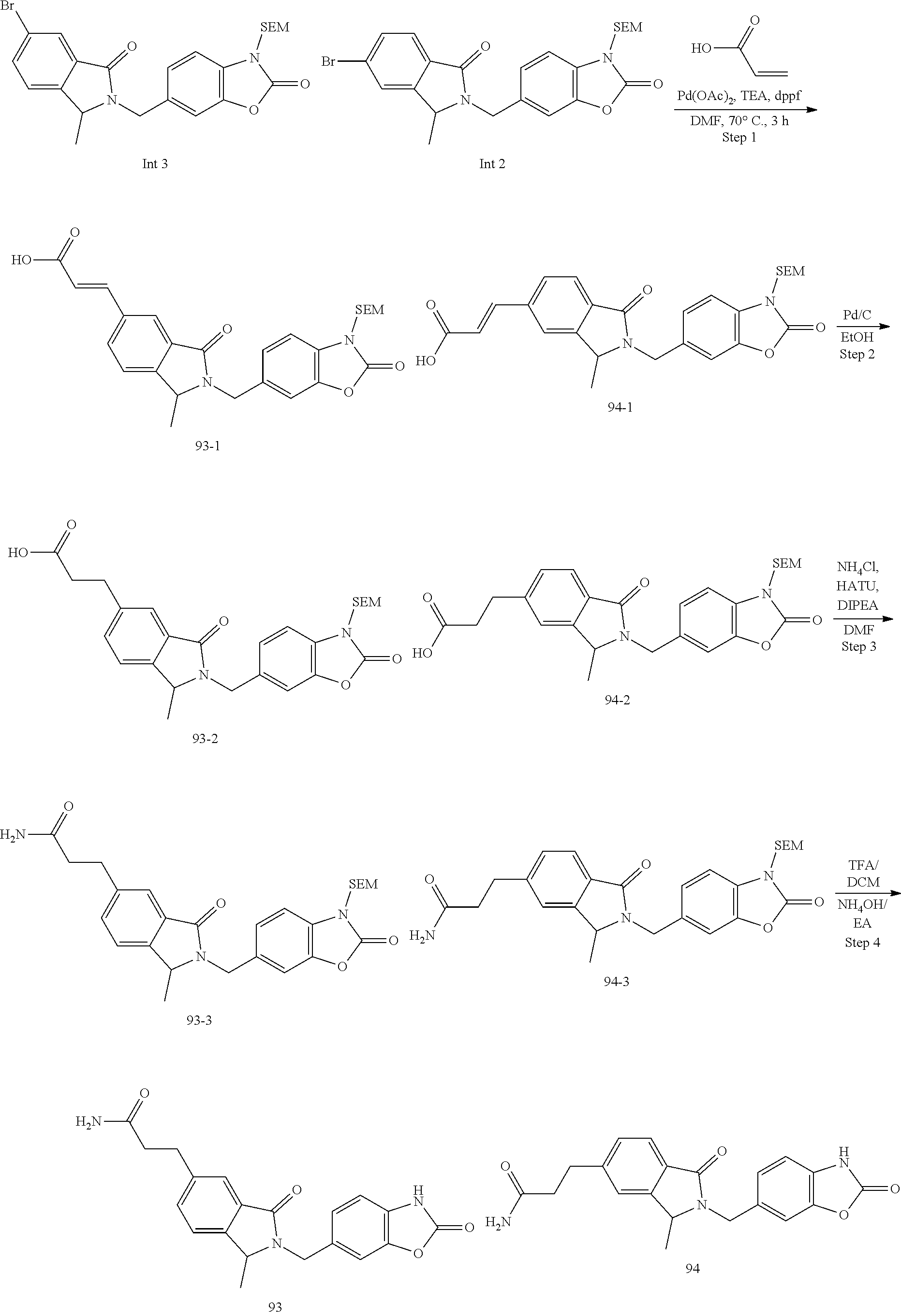

### Step 1. Mixture of (E)-3-(1-methyl-3-oxo-2-((2-oxo-3-((2-(trimethylsilyl)ethoxy) methyl)-2,3-dihydrobenzo[d]oxazol-6-yl)methyl)isoindolin-5-yl)acrylic acid (93-1) and (E)-3-(3-methyl-1-oxo-2-((2-oxo-3-((2-(trimethylsilyl)ethoxy)methyl)-2,3-dihydrobenzo[d]oxazol-6-yl)methyl)isoindolin-5-yl)acrylic acid (94-1)

To a solution of mixture of Int 2 and Int 3 (200 mg, 0.4 mmol) in DMF (3 mL) were added Pd(OAc)₂ (9 mg, 0.04 mmol), dppf(33 mg, 0.08 mmol), TEA (120 mg, 1.2 mmol) and acrylic acid (52 mg, 0.6 mmol) at r.t. under N₂. The resulting mixture was stirred for 3 h at 70° C. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=1/3) to afford a mixture of compound 93-1 and 94-1 (150 mg, 75.9%) as a yellow oil. MS (ESI) m/z 495 [M+H]⁺.

### Step 2. Mixture of 3-(1-methyl-3-oxo-2-((2-oxo-3-((2-(trimethylsilyl)ethoxy)methyl)-2,3-dihydrobenzo[d]oxazol-6-yl)methyl)isoindolin-5-yl)propanoic acid (93-2) and 3-(3-methyl-1-oxo-2-((2-oxo-3-((2-(trimethylsilyl)ethoxy)methyl)-2,3-dihydrobenzo[d]oxazol-6-yl) methyl)isoindolin-5-yl)propanoic acid (94-2)

To a solution of mixture of 93-1 and 94-1 (150 mg, 0.30 mmol) in EtOH (5 mL) was added Pd/C (15 mg). The mixture was evacuated and backfilled three times with hydrogen. The resulting mixture was stirred at r.t. overnight. The reaction mixture was filtered off and the filtrate was concentrated to afford a mixture of 93-2 and 94-2 (120 mg, 79.6%) as a yellow oil. MS (ESI) m/z 497 [M+H]⁺.

### Step 3. Mixture of 3-(1-methyl-3-oxo-2-((2-oxo-3-((2-(trimethylsilyl)ethoxy) methyl)-2,3-dihydrobenzo[d]oxazol-6-yl)methyl)isoindolin-5-yl)propenamide (93-3) and 3-(3-methyl-1-oxo-2-((2-oxo-3-((2-(trimethylsilyl)ethoxy)methyl)-2,3-dihydrobenzo[d]oxazol-6-yl) methyl)isoindolin-5-yl)propenamide (94-3)

To a solution of mixture of 93-2 and 94-2 (120 mg, 0.24 mmol) in DMF (5 mL) were added HATU (110 mg, 0.29 mmol), NH₄Cl (26 mg, 0.48 mmol) and DIPEA (62 mg, 0.48 mmol) at r.t. for 3 h, and then quenched with water, and extracted with EA (3×50 mL). The combined organic phase was washed with brine, dried over anhydrous Na₂SO₄ and concentrated to afford a mixture of 93-3 and 94-3 (80 mg, 66.8%) as a yellow oil. MS (ESI) m/z 496 [M+H]⁺.

### Step 4. 3-(1-methyl-3-oxo-2-((2-oxo-2,3-dihydrobenzo[d]oxazol-6-yl)methyl) isoindolin-5-yl)propenamide (93) and 3-(3-methyl-1-oxo-2-((2-oxo-2,3-dihydrobenzo[d]oxazol-6-yl) methyl)isoindolin-5-yl)propenamide (94)

To a solution of mixture of 93-3 and 94-3 (80 mg, 0.162 mmol) in DCM (5 mL) was added Trifluoroacetic acid (2 mL). The mixture was stirred at r.t. for 2h, and then concentrated under vacuum. The residue was dissolved with DCM (2 mL), and NH₄OH (1 mL) was added dropwise. The mixture was stirred at r.t. for 1 h. The reaction mixture was concentrated, and the residue was purified by Prep-HPLC to afford two compounds: compound 93 and compound 94. One of the two compounds was a white solid (7 mg, 23.7%), with RT=2.56 min (HPLC), MS (ESI) m/z 366 [M+H]⁺. ¹H NMR (400 MHz, DMSO-d₆) b 11.15 (s, 1H), 7.65-7.61 (m, 3H), 7.25 (dd, J=7.8, 1.5 Hz, 1H), 7.02-6.96 (m, 2H), 6.93-6.86 (m, 2H), 5.10 (d, J=15.2 Hz, 1H), 4.32-4.21 (m, 2H), 2.93 (t, J=7.5 Hz, 2H), 2.39 (t, J=7.6 Hz, 2H), 1.37 (d, J=6.7 Hz, 3H). The other one of the two compounds was a white solid (8 mg, 27%), with RT=2.80 min (HPLC), MS (ESI) m/z 366 [M+H]⁺. ¹H NMR (400 MHz, DMSO-d₆) δ 11.59 (s, 1H), 7.55 (d, J=1.4 Hz, 1H), 7.48-7.42 (m, 2H), 7.29 (s, 1H), 7.23 (d, J=1.5 Hz, 1H), 7.09-7.02 (m, 2H), 6.77 (s, 1H), 5.01 (d, J=15.2 Hz, 1H), 4.45-4.33 (m, 2H), 2.90 (t, J=7.5 Hz, 2H), 2.39 (t, J=7.6 Hz, 2H), 1.37 (d, J=6.7 Hz, 3H).

### 6-((3-methyl-1-oxo-5-((tetrahydro-2H-pyran-4-yl)amino)isoindolin-2-yl)methyl)benzo[d]oxazol-2(3H)-one (95)

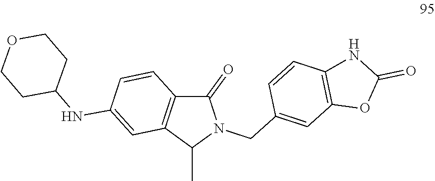 [SMILES: CC1c2cc(NC3CCOCC3)ccc2C(=O)N1Cc1ccc2[nH]c(=O)oc2c1]

The title compound 95 was prepared according to the procedure described for compound 47 as a white solid (23 mg, 13.5%). MS (ESI) m/z 394 [M+H]⁺. 1H NMR (400 MHz, DMSO-d₆) δ 11.60 (s, 1H), 7.37 (d, J=8.2 Hz, 1H), 7.18 (s, 1H), 7.06-7.01 (m, 2H), 6.70-6.62 (m, 2H), 4.93 (d, J=15.3 Hz, 11H), 4.31 (d, J=15.3 Hz, 11H), 4.23 (q, J=6.6 Hz, 11H), 3.90-3.81 (m, 2H), 3.57-3.46 (m, 1H), 3.46-3.35 (m, 2H), 1.91-1.82 (m, 2H), 1.47-1.34 (m, 2H), 1.32 (d, J=6.7 Hz, 3H).

### 6-((3-methyl-1-oxo-5-(pyrrolidin-1-ylmethyl)isoindolin-2-yl)methyl)benzo[d]oxazol-2(3H)-one (96)

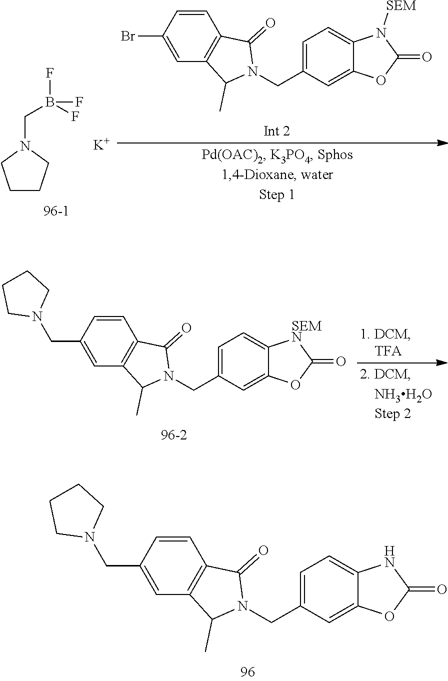

### Step 1. 6-((3-methyl-1-oxo-5-(pyrrolidin-1-ylmethyl)isoindolin-2-yl)methyl)-3-((2-(trimethylsilyl)ethoxy)methyl)benzo[d]oxazol-2(3H)-one (96-2)

To a solution of 6-((5-bromo-3-methyl-1-oxoisoindolin-2-yl)methyl)-3-((2-(trimethylsilyl) ethoxy)methyl)benzo[d]oxazol-2(3H)-one (250 mg, 0.49 mmol) in 1,4-Dioxane (2 mL) were added potassium trifluoro(pyrrolidin-1-ylmethyl)borate (228 mg, 1.19 mmol), SPhos (82 mg, 0.19 mmol), Pd(OAc)₂ (45 mg, 0.19 mmol), K₃PO₄ (421 mg, 1.99 mmol) and water (0.5 mL) under N₂. The mixture was stirred at 100° C. for 14 h. The mixture was diluted with EtOAc and washed with water. The organic layer was separated, dried over anhydrous Na₂SO₄ and concentrated under vacuum. The residue was purified by prep TLC (DCM/MeOH=25/1) to give 6-((3-methyl-1-oxo-5-(pyrrolidin-1-ylmethyl)isoindolin-2-yl)methyl)-3-((2-(trimethylsilyl) ethoxy)methyl)benzo[d]oxazol-2(3H)-one (85 mg, 33%) as a brown solid. MS (ESI) m/z 508 [M+H]⁺.

### Step 2. 6-((3-methyl-1-oxo-5-(pyrrolidin-1-ylmethyl)isoindolin-2-yl)methyl)benzo[d]oxazol-2(3H)-one (96)

To a solution of 6-((3-methyl-1-oxo-5-(pyrrolidin-1-ylmethyl)isoindolin-2-yl)methyl)-3-((2-(trimethylsilyl)ethoxy)methyl)benzo[d]oxazol-2(3H)-one (85 mg, 0.16 mmol) in DCM (3 mL) was added TFA (1 mL). The mixture was stirred for 3 h and then concentrated under vacuum. The residue was taken in DCM (3 mL) and ammonium hydroxide (1 mL) was added at 0° C. This suspension was allowed to stir for an additional 1 h. The mixture was concentrated and purified by prep HPLC to give 6-((3-methyl-1-oxo-5-(pyrrolidin-1-ylmethyl)isoindolin-2-yl)methyl) benzo[d]oxazol-2(3H)-one (18 mg, 27%) as a white solid. MS (ESI) m/z 378 [M+H]⁺. ¹H NMR (400 MHz, DMSO-d₆) δ 11.61 (s, 1H), 7.72 (d, J=7.7 Hz, 1H), 7.60 (s, 1H), 7.52 (dd, J=7.8, 1.4 Hz, 1H), 7.24 (d, J=1.5 Hz, 1H), 7.10 (dd, J=8.1, 1.5 Hz, 1H), 7.05 (d, J=8.0 Hz, 1H), 5.02 (d, J=15.2 Hz, 1H), 4.50-4.38 (m, 2H), 4.01 (s, 2H), 2.78 (d, J=6.4 Hz, 4H), 1.86-1.74 (m, 4H), 1.40 (d, J=6.6 Hz, 3H).

### 6-((3-benzyl-5-morpholino-1-oxoisoindolin-2-yl)methyl)benzo[d]oxazol-2(3H)-one (97)

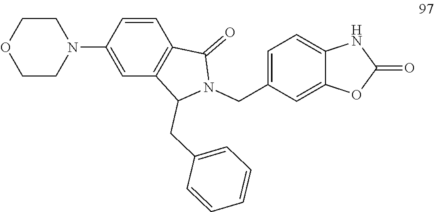 [SMILES: O=C1c2ccc(N3CCOCC3)cc2C(Cc2ccccc2)N1Cc1ccc2[nH]c(=O)oc2c1]

The title compound 97 was prepared according to the procedure described for compound 47 using 6-((3-benzyl-5-bromo-1-oxoisoindolin-2-yl)methyl)-3-((2-(trimethylsilyl)ethoxy)methyl) benzo[d]oxazol-2(3H)-one (preparation according to the procedure outlined for compound 106) as a starting material to afford a white solid (13 mg, 22%). MS (ESI) m/z 456 [M+H]⁺. ¹H NMR (400 MHz, DMSO-d₆) δ 11.74 (s, 1H), 7.41 (d, J=8.5 Hz, 1H), 7.23-7.11 (m, 3H), 7.08 (s, 1H), 7.05-6.94 (m, 5H), 6.79 (d, J=2.2 Hz, 1H), 5.08 (d, J=15.2 Hz, 1H), 4.54 (t, J=5.3 Hz, 1H), 4.31 (d, J=15.2 Hz, 1H), 3.72 (t, J=4.7 Hz, 4H), 3.35-3.29 (m, 1H), 3.21-3.08 (m, 5H).

### Mixture of 6-((5-amino-3-benzyl-1-oxoisoindolin-2-yl)methyl)benzo[d]oxazol-2(3H)-one (98) and 6-((5-amino-1-benzyl-3-oxoisoindolin-2-yl)methyl)benzo[d]oxazol-2(3H)-one (99)

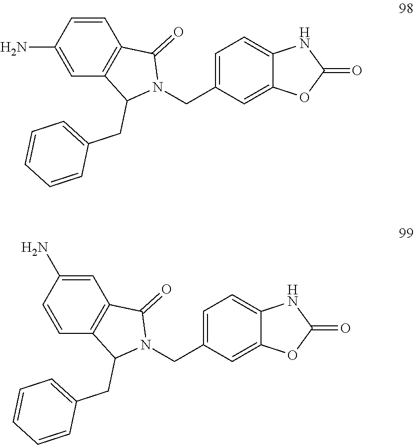 [SMILES: Nc1ccc2c(c1)C(Cc1ccccc1)N(Cc1ccc3[nH]c(=O)oc3c1)C2=O ; Nc1ccc2c(c1)C(=O)N(Cc1ccc3[nH]c(=O)oc3c1)C2Cc1ccccc1]

A mixture of 98 and 99 was prepared according to the procedure described for compound 75 and 76 as a yellow solid (31 mg, 48%). MS (ESI) m/z 386 [M+H]⁺. ¹H NMR (400 MHz, DMSO-d₆) δ 11.61 (s, 1H), 7.23 (d, J=8.0 Hz, 1H), 7.19-7.15 (m, 3H), 7.08 (d, J=2.0 Hz, 1H), 7.05-6.92 (m, 4H), 6.55 (dd, J=8.3, 2.0 Hz, 1H), 6.44 (d, J=1.9 Hz, 1H), 5.72 (s, 2H), 5.04 (d, J=15.2 Hz, 1H), 4.52-4.43 (m, 1H), 4.23 (d, J=15.2 Hz, 1H), 3.21-2.97 (m, 2H).

### 6-((3-benzyl-1-oxo-5-(1H-pyrazol-4-yl)isoindolin-2-yl)methyl)benzo[d]oxazol-2(3H)-one (100) and 6-((1-benzyl-3-oxo-5-(1H-pyrazol-4-yl)isoindolin-2-yl)methyl)benzo[d]oxazol-2(3H)-one (101)

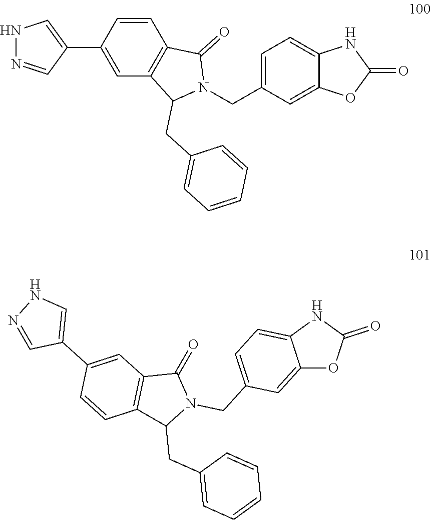 [SMILES: O=C1c2ccc(-c3cn[nH]c3)cc2C(Cc2ccccc2)N1Cc1ccc2[nH]c(=O)oc2c1 ; O=C1c2cc(-c3cn[nH]c3)ccc2C(Cc2ccccc2)N1Cc1ccc2[nH]c(=O)oc2c1]

The title compounds 100 and 101 were prepared according to the procedure described for compounds 57 and 58. After pre-HPLC separation, two compounds were obtained: compound 100 and compound 101. One of the two compounds was an off-white solid (10 mg, 21%), with RT=4.58 min (HPLC), MS (ESI) m/z 437 [M+H]⁺. ¹H NMR (400 MHz, DMSO-d₆) δ 13.05 (s, 1H), 11.65 (s, 1H), 8.18 (s, 1H), 7.91 (s, 1H), 7.66 (dd, J=8.0, 1.4 Hz, 1H), 7.57-7.50 (m, 2H), 7.19-7.11 (m, 4H), 7.05-6.97 (m, 4H), 5.12 (d, J=15.1 Hz, 1H), 4.70 (t, J=5.3 Hz, 1H), 4.41 (d, J=15.1 Hz, 1H), 3.39 (dd, J=14.0, 4.8 Hz, 1H), 3.22 (dd, J=14.0, 6.0 Hz, 1H). The other one of the two compounds was a light brown solid (8 mg, 17%), with RT=4.80 min (HPLC), MS (ESI) m/z 437 [M+H]⁺. ¹H NMR (400 MHz, DMSO-d₆) δ 12.97 (s, 1H), 11.65 (s, 1H), 8.25 (s, 1H), 8.03 (s, 1H), 7.87-7.74 (m, 2H), 7.25 (d, J=7.9 Hz, 1H), 7.22-7.11 (m, 4H), 7.09-6.96 (m, 4H), 5.16 (d, J=15.1 Hz, 1H), 4.68 (dd, J=6.3, 4.2 Hz, 1H), 4.44 (d, J=15.1 Hz, 1H), 3.38 (dd, J=14.0, 4.4 Hz, 1H) 3.15 (dd, J=14.0, 6.4 Hz, 1H).

### 6-((5-((2-azaspiro[3.3]heptan-6-yl)amino)-1-benzyl-3-oxoisoindolin-2-yl)methyl)benzo[d]oxazol-2(3H)-one (102) and 6-((5-((2-azaspiro[3.3]heptan-6-yl)amino)-3-benzyl-1-oxoisoindolin-2-yl)methyl)benzo[d]oxazol-2(3H)-one (103)

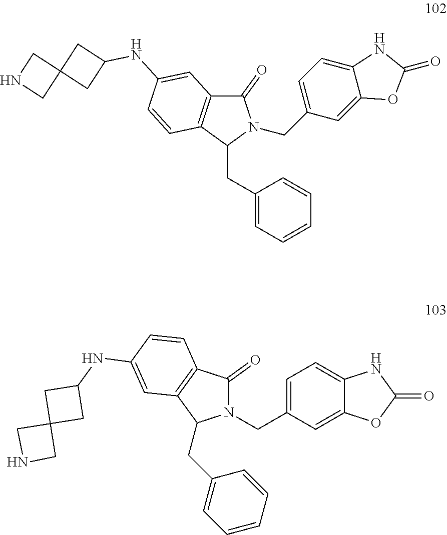 [SMILES: O=C1c2cc(NC3CC4(CNC4)C3)ccc2C(Cc2ccccc2)N1Cc1ccc2[nH]c(=O)oc2c1 ; O=C1c2ccc(NC3CC4(CNC4)C3)cc2C(Cc2ccccc2)N1Cc1ccc2[nH]c(=O)oc2c1]

The title compounds 102 and 103 were prepared according to the procedure described for compound 79 using a mixture of 6-((3-benzyl-5-bromo-1-oxoisoindolin-2-yl)methyl)-3-((2-(trimethylsilyl)ethoxy)methyl)benzo[d]oxazol-2(3H)-one and 6-((1-benzyl-5-bromo-3-oxoisoindolin-2-yl)methyl)-3-((2-(trimethylsilyl)ethoxy)methyl)benzo[d]oxazol-2(3H)-one as a starting material. After pre-HPLC separation, two compounds were obtained: compound 102 and compound 103. One of the two compounds was a light yellow solid (8 mg, 20%), with RT=2.99 min (HPLC), MS (ESI) m/z 481 [M+H]⁺. ¹H NMR (400 MHz, DMSO-d₆) δ 7.26 (d, J=8.4 Hz, 1H), 7.23-7.13 (m, 3H), 7.09-6.97 (m, 3H), 6.97-6.91 (m, 2H), 6.52-6.48 (m, 2H), 6.06 (s, 1H), 5.08 (d, J=15.0 Hz, 1H), 4.45 (dd, J=6.8, 4.4 Hz, 1H), 4.27 (d, J=15.0 Hz, 1H), 3.87-3.51 (m, 3H), 3.49-3.45 (m, 2H), 3.32 (dd, J=13.9, 4.3 Hz, 1H), 2.93 (dd, J=13.8, 6.6 Hz, 1H), 2.45-2.37 (m, 2H), 1.93-1.81 (m, 2H). The other one of the two compounds was an off-white solid (12 mg, 30%), with RT=3.37 min (HPLC), MS (ESI) m/z 481 [M+H]⁺. ¹H NMR (400 MHz, DMSO-d₆) δ 7.21-7.12 (m, 3H), 7.05-6.98 (m, 3H), 6.97-6.94 (m, 2H), 6.87 (d, J=8.0 Hz, 1H), 6.64-6.57 (m, 2H), 6.08 (d, J=6.4 Hz, 1H), 5.08 (d, J=15.1 Hz, 1H), 4.47 (dd, J=6.4, 4.4 Hz, 1H), 4.31 (d, J=15.1 Hz, 1H), 3.70-3.63 (m, 3H), 3.33-3.23 (m, 2H), 3.28 (dd, J=13.9, 4.4 Hz, 1H), 2.98 (dd, J=13.9, 7.1 Hz, 1H), 2.60-2.54 (m, 2H), 1.95-1.85 (m, 2H).

### N-(4-((1-oxoisoindolin-2-yl)methyl)phenyl)acetamide (104)

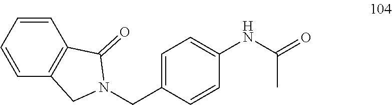 [SMILES: CC(=O)Nc1ccc(CN2Cc3ccccc3C2=O)cc1]

The title compound 104 was prepared according to the procedure described for compound 82 as a white solid (13 mg, 17%). MS (ESI) m/z 281 [M+H]⁺. ¹H NMR (400 MHz, DMSO-d₆) δ 9.94 (s, 1H), 7.60-7.47 (m, 1H), 7.63-7.54 (m, 3H), 7.54-7.45 (m, 2H), 7.20 (d, J=8.4 Hz, 2H), 4.66 (s, 2H), 4.34 (s, 2H), 2.02 (s, 3H).

### 1-methyl-3-(4-((1-oxoisoindolin-2-yl)methyl)phenyl)urea (105)

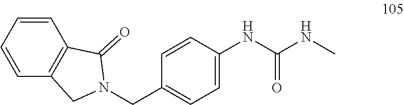 [SMILES: CNC(=O)Nc1ccc(CN2Cc3ccccc3C2=O)cc1]

The title compound 105 was prepared according to the procedure described for compound 82 as a light orange solid (47 mg, 36%). MS (ESI) m/z 296 [M+H]⁺. ¹H NMR (400 MHz, DMSO-d₆) δ 8.50 (s, 1H), 7.71 (d, J=7.6 Hz, 1H), 7.60-7.47 (m, 3H), 7.40-7.32 (m, 2H), 7.17-7.09 (m, 2H), 5.96 (q, J=4.6 Hz, 1H), 4.63 (s, 2H), 4.33 (s, 2H), 2.62 (d, J=4.6 Hz, 3H).

### 6-((1-benzyl-3-oxoisoindolin-2-yl)methyl)benzo[d]oxazol-2(3H)-one (106)

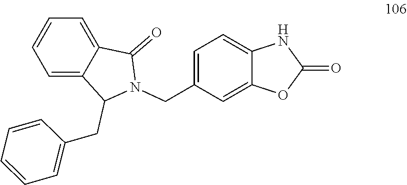 [SMILES: O=C1c2ccccc2C(Cc2ccccc2)N1Cc1ccc2[nH]c(=O)oc2c1]

The title compound 106 was prepared according to general procedure F as a white solid (116 mg, 43.6%). MS (ESI) m/z 371 [M+H]⁺. ¹H NMR (400 MHz, DMSO-d₆) δ 11.60 (s, 1H), 7.59 (d, J=7.5 Hz, 1H), 7.55-7.50 (m, 1H), 7.46-7.38 (m, 1H), 7.32 (d, J=7.6 Hz, 1H), 7.20 (s, 1H), 7.17-7.12 (m, 3H), 7.08-7.03 (m, 2H), 7.01-6.95 (m, 2H), 5.14 (d, J=15.0 Hz, 1H), 4.72 (dd, J=6.4, 4.4 Hz, 11H), 4.44 (d, J=15.1 Hz, 1H), 3.37 (dd, J=14.0, 4.3 Hz, 11H), 3.17 (dd, J=14.1, 6.3 Hz, 1H).

The intermediate 106-1 was prepared as follows:

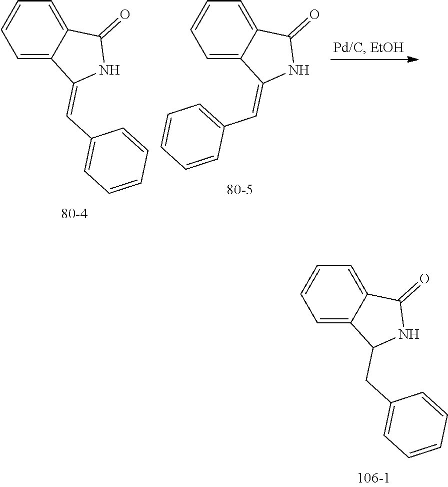

To a solution of mixture of 80-4 and 80-5 (1.5 g, 6.79 mmol) in EtOH (5 mL) was added Pd/C (150 mg). The mixture was evacuated and backfilled three times with hydrogen. The resulting mixture was stirred at r.t. overnight. The reaction mixture was filtered off and the filtrate was concentrated to afford 3-benzylisoindolin-1-one (1.3 g, 85.9%) as a white solid. MS (ESI) m/z 224 [M+H]⁺.

### (R)-6-((1-benzyl-3-oxoisoindolin-2-yl)methyl)benzo[d]oxazol-2(3H)-one (107) and (S)-6-((1-benzyl-3-oxoisoindolin-2-yl)methyl)benzo[d]oxazol-2(3H)-one (108)

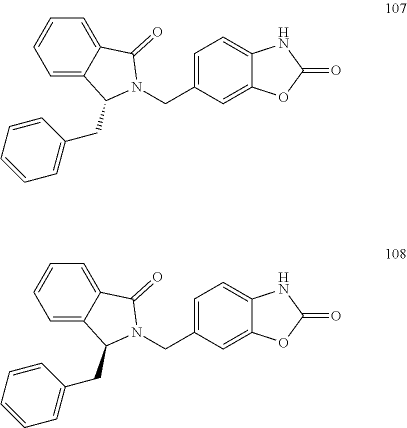 [SMILES: O=C1c2ccccc2[C@@H](Cc2ccccc2)N1Cc1ccc2[nH]c(=O)oc2c1 ; O=C1c2ccccc2[C@H](Cc2ccccc2)N1Cc1ccc2[nH]c(=O)oc2c1]

Chiral separation of compound 106 by chiral prep-HPLC (column: CHIRALPAK ID-3; column size: 2 cm×25 cm, 5 m; mobile phase A: MTBE; mobile phase B: MeOH:DCM) was conducted to afford two enantiomers: compound 107 and 108. One of the two enantiomers was a white solid, with RT=2.417 min (CHIRAL HPLC, column: CHIRALPAK ID-3; column size: 4.6*50 mm, 3 μm; mobile phase: MTBE (0.1% DEA):(MeOH:DCM=1:1)=95:5; flow: 1.0 mL/min). MS (ESI) m/z 371 [M+H]⁺. ¹H NMR (400 MHz, DMSO-d₆) δ 11.61 (s, 1H), 7.58 (d, J=7.5 Hz, 1H), 7.55-7.50 (m, 1H), 7.42 (t, J=7.4 Hz, 1H), 7.32 (d, J=7.5 Hz, 1H), 7.19 (s, 1H), 7.17-7.11 (m, 3H), 7.07-7.02 (m, 2H), 7.01-6.94 (m, 2H), 5.14 (d, J=15.1 Hz, 1H), 4.71 (t, J=5.4 Hz, 1H), 4.44 (d, J=15.1 Hz, 1H), 3.37 (dd, J=14.1, 4.3 Hz, 1H), 3.17 (dd, J=14.0, 6.4 Hz, 1H). The other one of the two enantiomers was a white solid, with RT=3.042 min, (CHIRAL HPLC, column: CHIRALPAK ID-3; column size: 4.6*50 mm, 3 μm; mobile phase: MTBE (0.1% DEA):(MeOH:DCM=1:1)=95:5; flow: 1.0 mL/min). MS (ESI) m/z 371 [M+H]⁺. ¹H NMR (400 MHz, DMSO-d₆) δ 11.61 (s, 1H), 7.58 (d, J=7.5 Hz, 1H), 7.55-7.50 (m, 1H), 7.42 (t, J=7.4 Hz, 1H), 7.32 (d, J=7.5 Hz, 1H), 7.19 (s, 1H), 7.17-7.10 (m, 3H), 7.08-7.03 (m, 2H), 6.99-6.95 (m, 2H), 5.14 (d, J=15.1 Hz, 1H), 4.71 (dd, J=6.4, 4.4 Hz, 1H), 4.44 (d, J=15.1 Hz, 1H), 3.37 (dd, J=14.1, 4.3 Hz, 1H), 3.17 (dd, J=14.0, 6.3 Hz, 1H).

### 6-((3-methyl-5-((2-methyl-2-azaspiro[3.3]heptan-6-yl)amino)-1-oxoisoindolin-2-yl)methyl) benzo[d]oxazol-2(3H)-one (109) and 6-((1-methyl-5-((2-methyl-2-azaspiro[3.3]heptan-6-yl)amino)-3-oxoisoindolin-2-yl)methyl)benzo[d]oxazol-2(3H)-one (110)

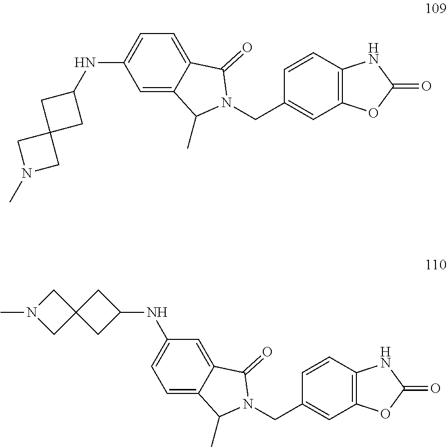 [SMILES: CC1c2cc(NC3CC4(C3)CN(C)C4)ccc2C(=O)N1Cc1ccc2[nH]c(=O)oc2c1 ; CC1c2ccc(NC3CC4(C3)CN(C)C4)cc2C(=O)N1Cc1ccc2[nH]c(=O)oc2c1]

The title compounds 109 and 110 were prepared according to the procedure described for compounds 50 and 51. After pre-HPLC separation, two compounds were obtained: compound 109 and compound 110. One of the two compounds was a white solid (4 mg, 4.4%), with RT=2.19 min (HPLC), MS (ESI) m/z 419 [M+H]⁺. ¹H NMR (400 MHz, DMSO-d₆) δ 7.37 (d, J=8.3 Hz, 1H), 7.18 (d, J=1.3 Hz, 1H), 7.06-7.01 (m, 2H), 6.58-6.52 (m, 2H), 6.48 (d, J=1.9 Hz, 1H), 4.93 (d, J=15.2 Hz, 1H), 4.30 (d, J=15.3 Hz, 1H), 4.22 (q, J=6.6 Hz, 1H), 3.79-3.69 (m, 1H), 3.43-3.37 (m, 2H), 3.30-3.25 (m, 2H), 2.59-2.52 (m, 2H), 2.29 (s, 3H), 2.00-1.86 (m, 2H), 1.31 (d, J=6.6 Hz, 3H). The other one of the two compounds was a white solid (6 mg, 6.5%), with RT=2.46 min (HPLC), MS (ESI) m/z 419 [M+H]⁺. ¹H NMR (400 MHz, DMSO-d₆) δ 7.26-7.08 (m, 2H), 7.10-6.90 (m, 2H), 6.83-6.56 (m, 2H), 6.12 (d, J=6.0 Hz, 1H), 4.97 (d, J=15.2 Hz, 1H), 4.34 (d, J=15.2 Hz, 1H), 4.24 (q, J=6.4 Hz, 1H), 3.77-3.69 (m, 1H), 3.53-3.26 (m, 4H), 2.60-2.53 (m, 2H), 2.37-2.28 (m, 2H), 1.93 (s, 3H), 1.30 (d, J=6.6 Hz, 3H).

### 6-((3-methyl-5-(1-methyl-1H-pyrazol-4-yl)-1-oxoisoindolin-2-yl)methyl)benzo[d]oxazol-2(3H)-one (1H) and 6-((1-methyl-5-(1-methyl-1H-pyrazol-4-yl)-3-oxoisoindolin-2-yl)methyl)benzo[d]oxazol-2(3H)-one (112)

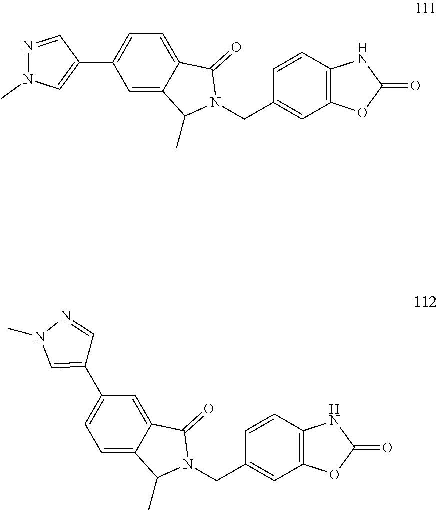 [SMILES: CC1c2cc(-c3cnn(C)c3)ccc2C(=O)N1Cc1ccc2[nH]c(=O)oc2c1 ; CC1c2ccc(-c3cnn(C)c3)cc2C(=O)N1Cc1ccc2[nH]c(=O)oc2c1]

The title compounds 111 and 112 were prepared according to the procedure described for compounds 57 and 58. After pre-HPLC separation, two compounds were obtained: compound 111 and compound 112. One of the two compounds was a white solid (12 mg, 10%), with RT=3.90 min (HPLC), MS (ESI) m/z 375 [M+H]⁺. ¹H NMR (400 MHz, DMSO-d₆) δ 11.69 (s, 1H), 8.25 (s, 1H), 7.96 (s, 1H), 7.78 (s, 1H), 7.71-7.64 (m, 2H), 7.22 (d, J=1.5 Hz, 1H), 7.08 (dd, J=8.0, 1.6 Hz, 1H), 7.03 (d, J=8.0 Hz, 1H), 4.99 (d, J=15.2 Hz, 1H), 4.51-4.34 (m, 2H), 3.87 (s, 3H), 1.42 (d, J=6.7 Hz, 3H). The other one of the two compounds was a white solid (6 mg, 5%), with RT=4.08 min (HPLC), MS (ESI) m/z 375 [M+H]⁺. ¹H NMR (400 MHz, DMSO-d₆) δ 11.67 (s, 1H), 8.27 (s, 1H), 7.96 (d, J=0.8 Hz, 1H), 7.87 (d, J=1.6 Hz, 1H), 7.80 (dd, J=7.9, 1.7 Hz, 1H), 7.55 (d, J=7.9 Hz, 1H), 7.22 (d, J=1.5 Hz, 1H), 7.08 (dd, J=8.0, 1.6 Hz, 1H), 7.03 (d, J=8.0 Hz, 1H), 5.02 (d, J=15.2 Hz, 1H), 4.46-4.36 (m, 2H), 3.87 (s, 3H), 1.39 (d, J=6.7 Hz, 3H).

### 6-((5-benzyl-3-methyl-1-oxoisoindolin-2-yl)methyl)benzo[d]oxazol-2(3H)-one (113)

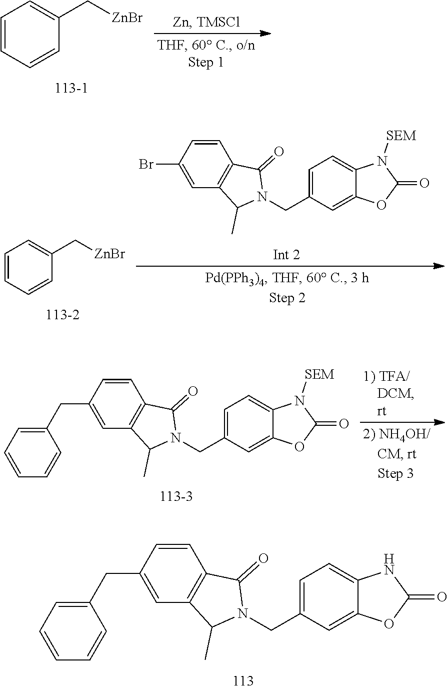

### Step 1. benzylzinc(H) bromide (113-2)

Under N₂, Zn powder (304 mg, 4.68 mmol) and dry THF (5 mL) were added to a flask. TMSCl (25 mg, 0.23 mmol) was then added to the above suspension and the resulting mixture was warmed to 50° C. (bromomethyl)benzene (400 mg, 2.34 mmol) in THF (1 mL) was added dropwise to the above mixture. The resulting mixture was stirred at 60° C. overnight. The reaction solution was cooled and used directly for the next step.

### Step 2. 6-((5-benzyl-3-methyl-1-oxoisoindolin-2-yl)methyl)-3-((2-(trimethylsilyl) ethoxy) methyl)benzo[d]oxazol-2(3H)-one (113-3)

6-((5-bromo-3-methyl-1-oxoisoindolin-2-yl)methyl)-3-((2-(trimethylsilyl)ethoxy)methyl) benzo[d]oxazol-2(3H)-one (300 mg, 0.8 mmol) and Pd(PPh₃)₄(93 mg, 0.08 mmol) in THF (5 mL) were charged to a dried flask. The flask was evacuated and backfilled three times with nitrogen, and then the above prepared benzylzinc(II) bromide was added dropwise. The resulting mixture was stirred at 60° C. for 3 h. The reaction solution was diluted with water (10 mL) and extracted with EtOAc (3×30 mL). The combined organic phase was dried and concentrated. The residue was purified by a silica gel column, eluted with petroleum ether and EtOAc (3:1) to afford the title compound (376 mg, 90.8%) as a yellow oil.

### Step 3. 6-((5-benzyl-3-methyl-1-oxoisoindolin-2-yl) methyl) benzo[d]oxazol-2(3H)-one (113)

To a solution of 6-((5-benzyl-3-methyl-1-oxoisoindolin-2-yl)methyl)-3-((2-(trimethylsilyl) ethoxy) methyl) benzo[d]oxazol-2(3H)-one (370 mg, 0.73 mmol) in DCM (10 mL) was added trifluoroacetic acid (2 mL). The resulting mixture was stirred at r.t. for 2 h. The reaction mixture was concentrated under vacuum. The residue was dissolved with DCM (5 mL), and then NH₄OH (1 mL) was added and stirred at r.t. for 1 h. The mixture was concentrated, and the residue was purified by Prep-HPLC to afford 6-((5-benzyl-3-methyl-1-oxoisoindolin-2-yl)methyl) benzo[d]oxazol-2(3H)-one (27 mg, 9.8%) as a white solid. MS (ESI) m/z 385 [M+H]⁺. ¹H NMR (400 MHz, DMSO-d₆) δ 11.60 (s, 1H), 7.63 (d, J=7.8 Hz, 1H), 7.47 (s, 1H), 7.35 (dd, J=7.8, 1.4 Hz, 1H), 7.31-7.15 (m, 6H), 7.09-6.99 (m, 2H), 4.99 (d, J=15.2 Hz, 1H), 4.44-4.35 (m, 2H), 4.04 (s, 2H), 1.37 (d, J=6.6 Hz, 3H).

### 6-((1-oxo-3-phenethylisoindolin-2-yl)methyl)benzo[d]oxazol-2(3H)-one (114)

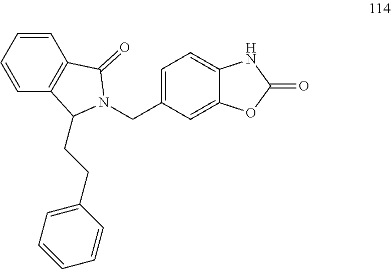 [SMILES: O=C1c2ccccc2C(CCc2ccccc2)N1Cc1ccc2[nH]c(=O)oc2c1]

The title compound 114 was prepared according to the procedure described for compound 67 as a white solid (175 mg, 39.1%). MS (ESI) m/z 385 [M+H]⁺. ¹H NMR (400 MHz, DMSO-d₆) δ 11.63 (s, 1H), 7.76 (d, J=7.5 Hz, 1H), 7.70-7.61 (m, 2H), 7.55-7.50 (m, 1H), 7.30 (d, J=1.5 Hz, 1H), 7.22-7.08 (m, 4H), 7.04 (d, J=7.9 Hz, 1H), 6.96-6.89 (m, 2H), 4.96 (d, J=15.1 Hz, 1H), 4.62 (dd, J=5.0, 3.1 Hz, 1H), 4.48 (d, J=15.1 Hz, 1H), 2.37-2.16 (m, 2H), 2.13-2.03 (m, 1H), 1.91-1.81 (m, 1H).

### 6-((5-((2,6-diazaspiro[3.3]heptan-2-yl)methyl)-3-methyl-1-oxoisoindolin-2-yl)methyl) benzo[d]oxazol-2(3H)-one (115)

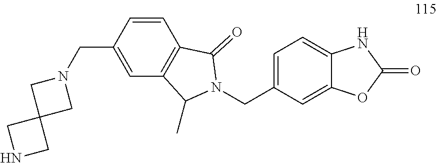 [SMILES: CC1c2cc(CN3CC4(CNC4)C3)ccc2C(=O)N1Cc1ccc2[nH]c(=O)oc2c1]

The title compound 115 was prepared according to the procedure described for compound 96 using potassium ((6-(tert-butoxycarbonyl)-2,6-diazaspiro[3.3]heptan-2-yl)methyl) trifluoroborate as a starting material to afford a white solid (16 mg, 7.5%). MS (ESI) m/z 405 [M+H]⁺. ¹H NMR (400 MHz, DMSO-d₆) δ 7.63 (d, J=7.8 Hz, 1H), 7.42 (s, l H), 7.25 (dd, J=8.0, 1.6 Hz, 1H), 7.17-7.14 (m, 1H), 7.05-6.94 (m, 2H), 5.00 (d, J=15.1 Hz, 1H), 4.43-4.31 (m, 2H), 3.66-3.52 (m, 8H), 3.22 (s, 2H), 1.37 (dd, J=6.7, 2.2 Hz, 3H).

### 6-((3-benzyl-5-cyclopropyl-1-oxoisoindolin-2-yl)methyl)benzo[d]oxazol-2(3H)-one (116)

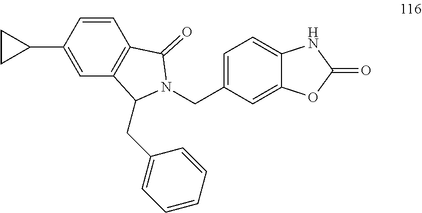 [SMILES: O=C1c2ccc(C3CC3)cc2C(Cc2ccccc2)N1Cc1ccc2[nH]c(=O)oc2c1]

The title compound 116 was prepared according to the procedure described for compound 100 as a white solid (33 mg, 25%). MS (ESI) m/z 411 [M+H]⁺. ¹H NMR (400 MHz, DMSO-d₆) δ 11.64 (s, 1H), 7.44 (d, J=7.8 Hz, 1H), 7.20-7.11 (m, 5H), 7.05-7.02 (m, 2H), 7.00-6.96 (m, 2H), 6.83 (d, J=1.5 Hz, 1H), 5.11 (d, J=15.2 Hz, 1H), 4.61 (dd, J=6.4, 4.4 Hz, 1H), 4.39 (d, J=15.2 Hz, 1H), 3.36 (dd, J=14.0, 4.4 Hz, 1H), 3.07 (dd, J=14.0, 6.4 Hz, 1H), 1.98-1.91 (m, 1H), 1.05-0.92 (m, 2H), 0.67-0.59 (m, 2H).

### 6-((1-benzyl-3-oxo-5-((tetrahydro-2H-pyran-4-yl)amino)isoindolin-2-yl)methyl)benzo[d]oxazol-2(3H)-one (117) and 6-((3-benzyl-1-oxo-5-((tetrahydro-2H-pyran-4-yl)amino) isoindolin-2-yl)methyl)benzo[d]oxazol-2(3H)-one (118)

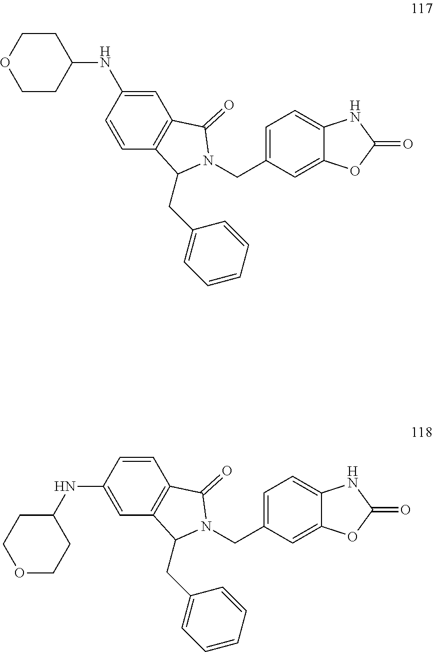 [SMILES: O=C1c2cc(NC3CCOCC3)ccc2C(Cc2ccccc2)N1Cc1ccc2[nH]c(=O)oc2c1 ; O=C1c2ccc(NC3CCOCC3)cc2C(Cc2ccccc2)N1Cc1ccc2[nH]c(=O)oc2c1]

The title compounds 117 and 118 were prepared according to the procedure described for compound 95 using a mixture of 6-((3-benzyl-5-bromo-1-oxoisoindolin-2-yl)methyl)-3-((2-(trimethylsilyl)ethoxy)methyl)benzo[d]oxazol-2(3H)-one and 6-((1-benzyl-5-bromo-3-oxoisoindolin-2-yl)methyl)-3-((2-(trimethylsilyl)ethoxy)methyl)benzo[d]oxazol-2(3H)-one as a starting material. After pre-HPLC separation, two compounds were obtained: compound 117 and compound 118. One of the two compounds was an off-white solid (5 mg, 14%), with RT=5.06 min (HPLC), MS (ESI) m/z 470 [M+H]⁺. ¹H NMR (400 MHz, DMSO-d₆) δ 11.68 (s, 1H), 7.23-7.15 (m, 3H), 7.13 (s, 1H), 7.06-6.97 (m, 4H), 6.88 (d, J=8.2 Hz, 1H), 6.77-6.70 (m, 2H), 5.77 (d, J=8.0 Hz, 1H), 5.09 (d, J=15.2 Hz, 1H), 4.49 (dd, J=6.4, 4.4 Hz, 1H), 4.36 (d, J=15.2 Hz, 1H), 3.89-3.80 (m, 2H), 3.46-3.38 (m, 3H), 3.29 (dd, J=14.0, 4.4 Hz, 1H), 2.99 (dd, J=14.0, 6.4 Hz, 1H), 1.89-1.80 (m, 2H), 1.42-1.26 (m, 2H). The other one of the two compounds was a white solid (6 mg, 17%) with RT=4.94 min (HPLC), MS (ESI) m/z 470 [M+H]⁺. ¹H NMR (400 MHz, DMSO-d₆) δ 11.65 (s, 1H), 7.27 (d, J=8.4 Hz, 1H), 7.23-7.14 (m, 3H), 7.13-6.96 (m, 5H), 6.60 (dd, J=8.3, 2.0 Hz, 11H), 6.24-6.15 (m, 2H), 5.04 (d, J=15.2 Hz, 1H), 4.48 (dd, J=7.2, 4.4 Hz, 1H), 4.30 (d, J=15.2 Hz, 1H), 3.91-3.79 (m, 2H), 3.37-3.35 (m, 1H), 3.25-3.20 (m, 3H), 2.91 (dd, J=13.8, 7.2 Hz, 1H), 1.83-1.70 (m, 2H), 1.4-1.21 (m, 2H).

### 6-((5-hydroxy-3-methyl-1-oxoisoindolin-2-yl)methyl)benzo[d]oxazol-2(3H)-one (119) and 6-((5-hydroxy-1-methyl-3-oxoisoindolin-2-yl)methyl)benzo[d]oxazol-2(3H)-one (120)

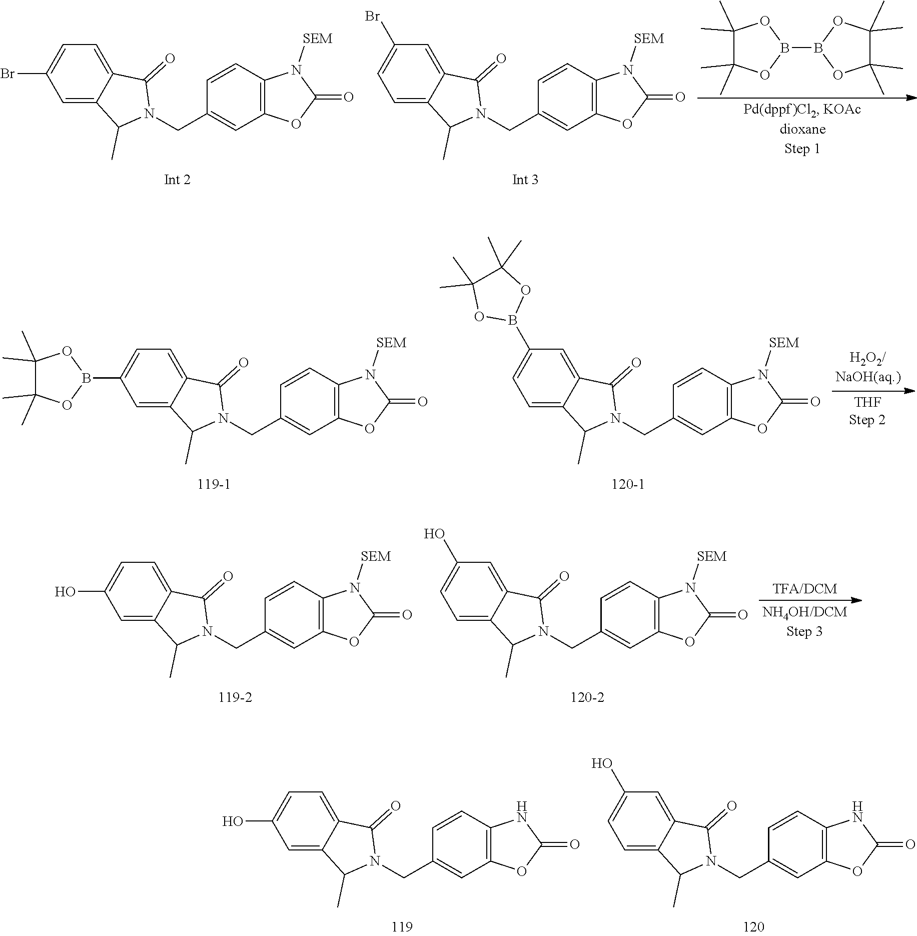

### Step 1. Mixture of 6-((3-methyl-1-oxo-5-(4,4,5,5-tetramethyl-1,3,2-dioxaborolan-2-yl)isoindolin-2-yl)methyl)-3-((2-(trimethylsilyl)ethoxy)methyl)benzo[d]oxazol-2(3H)-one (119-1) and 6-((1-methyl-3-oxo-5-(4,4,5,5-tetramethyl-1,3,2-dioxaborolan-2-yl)isoindolin-2-yl)methyl)-3-((2-(trimethylsilyl)ethoxy)methyl) benzo[d]oxazol-2(3H)-one (120-1)

To a solution of mixture of Int 2 and Int 3 (500 mg, 1 mmol) in 1,4-dioxane (10 mL) were added Pd(dppf)C₁₂ (81 mg, 0.1 mmol), KOAc (196 mg, 2. mmol) and 4,4,4′,4′,5,5,5′,5′-octamethyl-2,2′-bi(1,3,2-dioxaborolane) (380 mg, 1.5 mmol) at r.t. under N₂. The resulting mixture was stirred for 1 h at 100′C using a Biotage microwave reactor. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=1/4) to afford a mixture of 119-1 and 120-1 (400 mg, 72.6%) as a yellow oil. MS (ESI) m/z 551 [M+H]⁺.

### Step 2. Mixture of 6-((5-hydroxy-3-methyl-1-oxoisoindolin-2-yl)methyl)-3-((2-(trimethylsilyl)ethoxy)methyl)benzo[d]oxazol-2(3H)-one (119-2) and 6-((5-hydroxy-1-methyl-3-oxoisoindolin-2-yl)methyl)-3-((2-(trimethylsilyl)ethoxy)methyl)benzo[d]oxazol-2(3H)-one (120-2)

To a solution of mixture of 119-1 and 120-1 (200 mg, 0.36 mmol) in THE (10 mL) were added NaOH (1M, 0.4 mL) and H₂O₂(30%, 0.4 mL) at 0° C., and stirred for 1 h. Then quenched with water, extracted with EA (3×50 mL). The combined organic phase was washed with brine, dried over anhydrous Na₂SO₄ and concentrated to afford a mixture of 119-2 and 120-2 (100 mg, 62.4%) as a yellow oil. MS (ESI) m/z 441 [M+H]⁺.

### Step 3. 6-((5-hydroxy-3-methyl-1-oxoisoindolin-2-yl)methyl)benzo[d]oxazol-2(3H)-one (119) and 6-((5-hydroxy-1-methyl-3-oxoisoindolin-2-yl)methyl)benzo[d]oxazol-2(3H)-one (120)

To a solution of mixture of 119-2 and 120-2 (100 mg, 0.23 mmol) in DCM (5 mL) was added trifluoroacetic acid (2 mL). The mixture was stirred at r.t. for 2h, and then concentrated under vacuum. The residue was dissolved with DCM (4 mL), and NH₄OH (2 mL) was added dropwise. The mixture was stirred at r.t. for 1 h. The reaction mixture was concentrated, and the residue was purified by Prep-HPLC to afford two compounds: compound 119 and compound 120. One of the two compounds was a white solid (2 mg, 5.7%), with RT=3.35 min (HPLC), MS (ESI) m/z 311 [M+H]⁺. ¹H NMR (400 MHz, DMSO-d) δ 10.26 (s, 1H), 7.51 (d, J=7.6 Hz, 1H), 7.17 (s, 1H), 7.07-6.97 (m, 2H), 6.88-6.84 (m, 2H), 4.95 (d, J=15.2 Hz, 1H), 4.37-4.25 (m, 2H), 1.33 (d, J=6.7 Hz, 3H). The other one of the two compounds was a white solid (3 mg, 8.5%), with RT=3.54 min (HPLC), MS (ESI) m/z 311 [M+H]⁺. ¹H NMR (400 MHz, DMSO-d₆) δ 9.85 (s, 11H), 7.34 (d, J=8.2 Hz, 1H), 7.15 (s, 1H), 7.05-6.94 (m, 4H), 4.97 (d, J=15.1 Hz, 11H), 4.38-4.25 (m, 2H), 1.33 (d, J=6.6 Hz, 3H).

### 6-((3-methyl-1-oxo-5-phenoxyisoindolin-2-yl)methyl)benzo[d]oxazol-2(3H)-one (121) and 6-((1-methyl-3-oxo-5-phenoxyisoindolin-2-yl)methyl)benzo[d]oxazol-2(3H)-one (122)

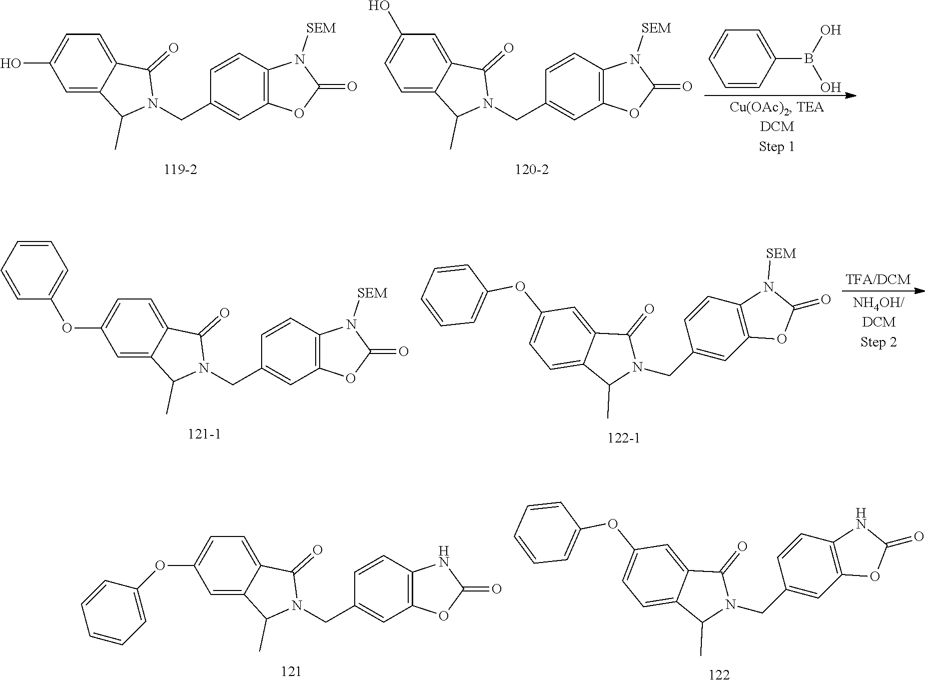

### Step 1. Mixture of 6-((3-methyl-1-oxo-5-phenoxyisoindolin-2-yl)methyl)-3-((2-(trimethylsilyl)ethoxy)methyl)benzo[d]oxazol-2(3H)-one (121-1) and 6-((1-methyl-3-oxo-5-phenoxyisoindolin-2-yl)methyl)-3-((2-(trimethylsilyl)ethoxy)methyl)benzo[d]oxazol-2(3H)-one (122-1)

To a solution of mixture of 119-2 and 120-2 (200 mg, 0.45 mmol) in DCM (10 ml) were added phenylboronic acid (111 mg, 0.91 mmol), Cu(OAc)₂ (17 mg, 0.09 mmol) and TEA (92 mg, 0.91 mmol) at r.t. under oxygen atmosphere. The resulting mixture was stirred at r.t. overnight. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=1/4) to afford a mixture of 121-1 and 122-1 (100 mg, 42.7%) as a yellow oil. MS (ESI) m/z 517 [M+H]⁺.

### Step 2. 6-((3-methyl-1-oxo-5-phenoxyisoindolin-2-yl)methyl)benzo[d]oxazol-2(3H)-one (121) and 6-((1-methyl-3-oxo-5-phenoxyisoindolin-2-yl)methyl)benzo[d]oxazol-2(3H)-one (122)

To a solution of mixture of 121-1 and 122-1 (100 mg, 0.19 mmol) in DCM (5 mL) was added trifluoroacetic acid (2 mL). The mixture was stirred at r.t. for 2h, and then concentrated under vacuum. The residue was dissolved with DCM (2 mL), and NH₄OH (1 mL) was added dropwise. The mixture was stirred at r.t. for 1 h. The reaction mixture was concentrated, and the residue was purified by Prep-HPLC afford two compounds: compound 121 and compound 122. One of the two compounds was a white solid (9 mg, 24%), with RT=4.04 min (HPLC), MS (ESI) m/z 387 [M+H]⁺. ¹H NMR (400 MHz, DMSO-d,) δ 7.60 (d, J=8.2 Hz, 1H), 7.49-7.38 (m, 2H), 7.30 (dd, J=8.3, 2.4 Hz, 1H), 7.21 (dt, J=14.8, 1.1 Hz, OH), 7.21 (s, 2H), 7.13 (d, J=2.4 Hz, 1H), 7.11-7.02 (m, 3H), 7.02 (d, J=7.9 Hz, 1H), 5.00 (d, J=15.2 Hz, 1H), 4.40 (dd, J=14.7, 8.6 Hz, 2H), 1.38 (dd, J=13.6, 6.7 Hz, 3H). The other one of the two compounds was a white solid (9 mg, 24%), with RT=3.94 min (HPLC), MS (ESI) m/z 311 [M+H]⁺.

### 6-((3-benzyl-5-(morpholinomethyl)-1-oxoisoindolin-2-yl)methyl)benzo[d]oxazol-2(3H)-one (123)

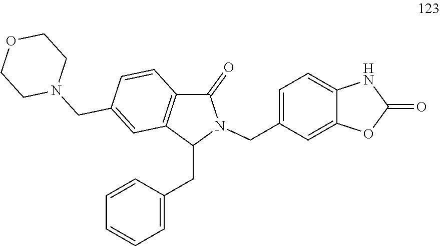 [SMILES: O=C1c2ccc(CN3CCOCC3)cc2C(Cc2ccccc2)N1Cc1ccc2[nH]c(=O)oc2c1]

The title compound 123 was prepared according to the procedure described for compound 96 using 6-((3-benzyl-5-bromo-1-oxoisoindolin-2-yl)methyl)-3-((2-(trimethylsilyl)ethoxy)methyl) benzo[d]oxazol-2(3H)-one and hydrogen trifluoro(morpholinomethyl)borate as starting materials to afford a white solid (10 mg, 310%). MS (EST) m/z 470 [M+H]⁺. ¹H NMR (400 MHz, DMSO-d₆) δ 11.70 (s, 1H), 7.51 (d, J=7.7 Hz, 1H), 7.32 (d, J=7.7 Hz, 1H), 7.28 (s, 1H), 7.20 (d, J=1.5 Hz, 1H), 7.13-7.09 (m, 3H), 7.08-7.02 (m, 2H), 6.97-6.93 (m, 2H), 5.15 (d, J=15.2 Hz, 1H), 4.68 (dd, J=6.0, 4.4 Hz, 11H), 4.45 (d, J=15.2 Hz, 1H), 3.57 (t, J=4.6 Hz, 4H), 3.48 (d, J=2.5 Hz, 2H), 3.36 (dd, J=14.0, 4.4 Hz, 1H), 3.17 (dd, J=14.0, 6.0 Hz, 1H), 2.30 (d, J=4.8 Hz, 4H).

### 6-((1-(2-methylbenzyl)-3-oxoisoindolin-2-yl)methyl)benzo[d]oxazol-2(3H)-one (124)

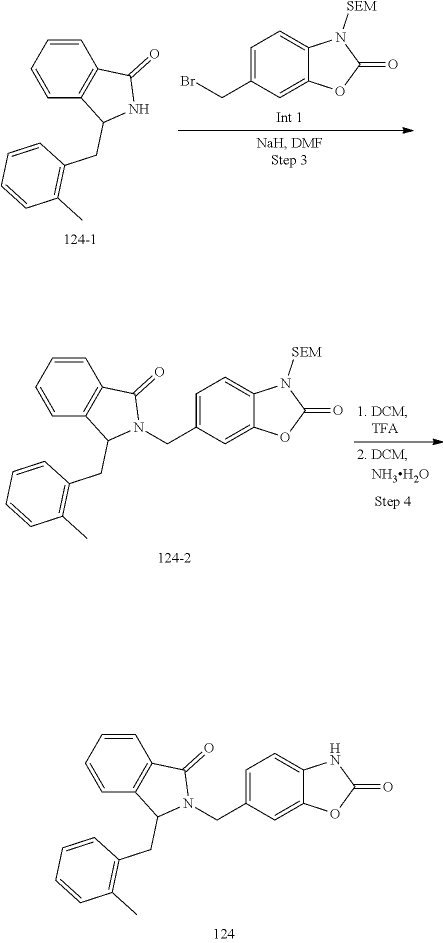 [SMILES: Cc1ccccc1CC1NC(=O)c2ccccc21 ; C[Si](C)(C)CCOCn1c(=O)oc2cc(CBr)ccc21 ; Cc1ccccc1CC1c2ccccc2C(=O)N1Cc1ccc2c(c1)oc(=O)n2COCC[Si](C)(C)C ; Cc1ccccc1CC1c2ccccc2C(=O)N1Cc1ccc2[nH]c(=O)oc2c1]

The intermediate 124-1 was prepared according to the procedure described for Int 12 as a yellow sloid.

### Step 1. 6-((1-(2-methylbenzyl)-3-oxoisoindolin-2-yl)methyl)-3-((2-(trimethylsilyl)ethoxy) methyl)benzo[d]oxazol-2(3H)-one (124-2)

To a solution of 3-(2-methylbenzyl)isoindolin-1-one (80 mg, 0.33 mmol) in DMF (2 mL) was added NaH (9 mg, 0.37 mmol) at 0° C. under N₂. The mixture was stirred for 30 min. 6-(bromomethyl)-3-((2-(trimethylsilyl)ethoxy)methyl)benzo[d]oxazol-2(3H)-one (133 mg, 0.37 mmol) was added and the reaction allowed to warm to r.t. This suspension was allowed to stir for additional 2 h. Water was added at 0° C. and then extracted with EtOAc. The organic layer was separated, dried over anhydrous Na₂SO₄ and concentrated under vacuum. The residue was purified by prep TLC (DCM/MeOH=25/1) to give 6-((1-(2-methylbenzyl)-3-oxoisoindolin-2-yl)methyl)-3-((2-(trimethylsilyl)ethoxy)methyl)benzo[d]oxazol-2(3H)-one (60 mg, 34%) as a yellow solid. MS (ESI) m/z 515 [M+H]⁺.

### Step 2. 6-((1-(2-methylbenzyl)-3-oxoisoindolin-2-yl)methyl)benzo[d]oxazol-2(3H)-one (124)

To a solution of 6-((1-(2-methylbenzyl)-3-oxoisoindolin-2-yl)methyl)-3-((2-(trimethylsilyl) ethoxy)methyl)benzo[d]oxazol-2(3H)-one (60 mg, 0.11 mmol) in DCM (3 mL) was added TFA (1 mL). The mixture was stirred for 3 h and then concentrated under vacuum. The residue was taken in DCM (3 mL) and ammonium hydroxide (1 mL) was added at 0° C. This suspension was allowed to stir for an additional 1 h. The mixture was concentrated and purified by prep HPLC to give 6-[[1-(o-tolylmethyl)-3-oxo-isoindolin-2-yl]methyl]-3H-1,3-benzoxazol-2-one (25 mg, 56%) as an off-white solid. MS (ESI) m/z 385 [M+H]⁺. ¹H NMR (400 MHz, DMSO-d₆) δ 11.68 (s, 1H), 7.76-7.69 (m, 1H), 7.49-7.40 (m, 2H), 7.22-7.11 (m, 3H), 7.11-7.07 (m, 1H), 7.07-7.00 (m, 2H), 6.96 (dd, J=8.0, 2.0 Hz, 1H), 6.78 (d, J=6.8 Hz, 1H), 5.15 (d, J=15.2 Hz, 1H), 4.64 (dd, J=8.4, 6.0 Hz, 1H), 4.33 (d, J=15.1 Hz, 1H), 3.42 (dd, J=13.6, 6.0 Hz, 1H), 2.75 (dd, J=13.6, 8.4 Hz, 1H), 2.09 (s, 3H).

### 6-((5-cyclopropyl-3-methyl-1-oxoisoindolin-2-yl)methyl)benzo[d]oxazol-2(3H)-one (125)

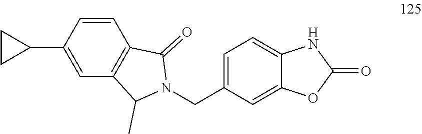 [SMILES: CC1c2cc(C3CC3)ccc2C(=O)N1Cc1ccc2[nH]c(=O)oc2c1]

The title compound 125 was prepared according to the procedure described for compound 57 as a white solid (7 mg, 5%). MS (ESI) m/z 335 [M+H]⁺. ¹H NMR (400 MHz, DMSO-d₆) δ 7.56 (d, J=7.8 Hz, 1H), 7.26 (s, 1H), 7.21-7.18 (m, 2H), 7.07-7.01 (m, 2H), 4.98 (d, J=15.2 Hz, 1H), 4.39-4.32 (m, 2H), 2.07-1.99 (m, 1H), 1.37 (d, J=6.7 Hz, 3H), 1.03-1.00 (m, 2H), 0.80-0.70 (m, 2H).

### (R)-6-((5-cyclopropyl-3-methyl-1-oxoisoindolin-2-yl)methyl)benzo[d]oxazol-2(3H)-one (126) and (S)-6-((5-cyclopropyl-3-methyl-1-oxoisoindolin-2-yl)methyl)benzo[d]oxazol-2(3H)-one (127)

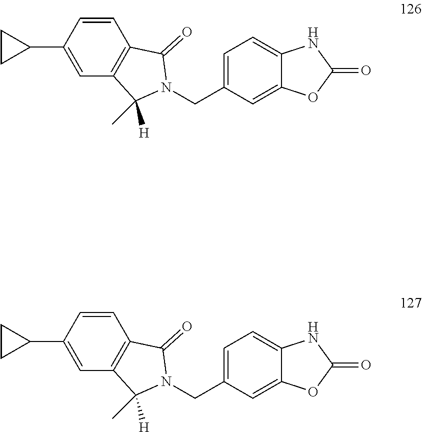 [SMILES: C[C@@H]1c2cc(C3CC3)ccc2C(=O)N1Cc1ccc2[nH]c(=O)oc2c1 ; C[C@H]1c2cc(C3CC3)ccc2C(=O)N1Cc1ccc2[nH]c(=O)oc2c1]

The title compound 126 and compound 127 were prepared, respectively, according to the procedure described for compound 125. The stereoisomer of Int 10 or Int 11 that has RT of 1.728 min was used as a starting material to afford one white solid (50 mg, 37%) that has the following characterization: MS (ESI) m/z 335 [M+H]⁺. ¹H NMR (400 MHz, DMSO-d₆) δ 11.60 (s, 1H), 7.56 (d, J=7.8 Hz, 11H), 7.26 (s, 11H), 7.22-7.18 (m, 2H), 7.08-7.02 (m, 2H), 4.98 (d, J=15.2 Hz, 1H), 4.40-4.33 (m, 2H), 2.07-1.99 (m, 1H), 1.37 (d, J=6.7 Hz, 3H), 1.03-1.00 (m, 2H), 0.78-0.73 (m, 2H). The stereoisomer of Int 10 or Int 11 that has RT of 1.969 min was used as a starting material to afford one white solid (43 mg, 33%) that has the following characterization: MS (ESI) m/z 335 [M+H]⁺. 1H NMR (400 MHz, DMSO-d₆) δ 11.63 (s, 1H), 7.56 (d, J=7.9 Hz, 1H), 7.26 (s, 1H), 7.21-7.18 (m, 2H), 7.08-7.01 (m, 2H), 4.98 (d, J=15.2 Hz, 1H), 4.40-4.33 (m, 2H), 2.06-2.00 (m, 1H), 1.37 (d, J=6.7 Hz, 3H), 1.03-1.01 (m, 2H), 0.80-0.71 (m, 2H).

### 6-((5-(3-methoxyazetidin-1-yl)-3-methyl-1-oxoisoindolin-2-yl)methyl)benzo[d]oxazol-2(3H)-one (128)

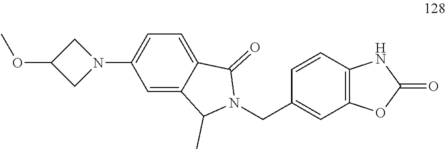 [SMILES: COC1CN(c2ccc3c(c2)C(C)N(Cc2ccc4[nH]c(=O)oc4c2)C3=O)C1]

The title compound 128 was prepared according to the procedure described for compound 51 as a white solid (3 mg, 4%). MS (ESI) m/z 380 [M+H]⁺. ¹H NMR (400 MHz, DMSO-d₆) δ 11.60 (s, 1H), 7.48 (d, J=8.2 Hz, 1H), 7.18 (s, 1H), 7.08-6.98 (m, 2H), 6.53-6.43 (m, 2H), 4.94 (d, J=15.3 Hz, 1H), 4.38-4.29 (m, 2H), 4.27 (q, J=6.5 Hz, 1H), 4.14-4.08 (m, 2H), 3.72-3.66 (m, 2H), 3.24 (s, 3H), 1.34 (d, J=6.6 Hz, 3H).

### 6-((1-oxo-3-(pyridin-2-ylmethyl)isoindolin-2-yl)methyl)benzo[d]oxazol-2(3H)-one (129)

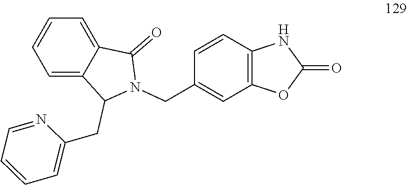 [SMILES: O=C1c2ccccc2C(Cc2ccccn2)N1Cc1ccc2[nH]c(=O)oc2c1]

The title compound 129 was prepared according to the procedure described for compound 124 as a white solid (16 mg, 23%). MS (ESI) m/z 372 [M+H]⁺. ¹H NMR (400 MHz, DMSO-d₆) δ 11.65 (s, 1H), 8.50-8.43 (m, 1H), 7.68-7.56 (m, 2H), 7.54-7.40 (m, 2H), 7.25-7.18 (m, 11H), 7.15-7.08 (m, 2H), 7.04-6.95 (m, 3H), 5.11 (d, J=15.3 Hz, 1H), 4.95 (dd, J=6.9, 4.9 Hz, 1H), 4.42 (d, J=15.3 Hz, 11H), 3.51 (dd, J=14.0, 5.0 Hz, 11H), 3.18 (dd, J=14.0, 7.0 Hz, 11H).

### 6-((1-oxo-3-(pyridin-4-ylmethyl)isoindolin-2-yl)methyl)benzo[d]oxazol-2(3H)-one (130)

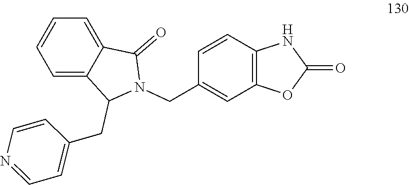 [SMILES: O=C1c2ccccc2C(Cc2ccncc2)N1Cc1ccc2[nH]c(=O)oc2c1]

The title compound 130 was prepared according to the procedure described for compound 124 as a white solid (4 mg, 4.5%). MS (ESI) m/z 372 [M+H]⁺. ¹H NMR (400 MHz, DMSO-d₆) δ 8.31-8.27 (m, 2H), 7.59-7.54 (m, 2H), 7.49-7.42 (m, 2H), 7.21 (s, 1H), 7.08-6.98 (m, 2H), 6.96-6.88 (m, 2H), 5.14 (d, J=15.0 Hz, 1H), 4.80 (t, J=4.9 Hz, 1H), 4.48 (d, J=15.0 Hz, 1H), 4.44-4.37 (m, 1H), 3.39 (dd, J=14.0, 4.4 Hz, 1H).

### 3-((3-oxo-2-((2-oxo-2,3-dihydrobenzo[d]oxazol-6-yl)methyl)isoindolin-1-yl)methyl) benzonitrile (131)

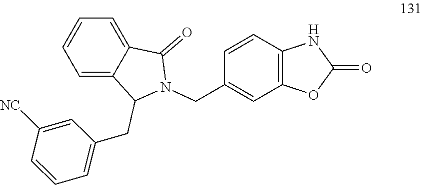 [SMILES: N#Cc1cccc(CC2c3ccccc3C(=O)N2Cc2ccc3[nH]c(=O)oc3c2)c1]

The title compound 131 was prepared according to the procedure described for compound 124 as a white solid (28 mg, 32%). MS (ESI) m/z 396 [M+H]⁺. ¹H NMR (400 MHz, DMSO-d₆) δ 11.61 (s, 1H), 7.63-7.54 (m, 3H), 7.51-7.40 (m, 2H), 7.38-7.28 (m, 2H), 7.24 (d, J=7.9 Hz, 1H), 7.19 (s, 1H), 7.10-7.00 (m, 2H), 5.09 (d, J=15.1 Hz, 1H), 4.82 (t, J=4.9 Hz, 1H), 4.52 (d, J=15.2 Hz, 1H), 3.45-3.34 (m, 2H).

### 6-((5-((2-azaspiro[3.3]heptan-6-yl)oxy)-3-methyl-1-oxoisoindolin-2-yl)methyl)benzo[d]oxazol-2(3H)-one (132) and 6-((5-((2-azaspiro[3.3]heptan-6-yl)oxy)-1-methyl-3-oxoisoindolin-2-yl)methyl)benzo[d]oxazol-2(3H)-one (133)

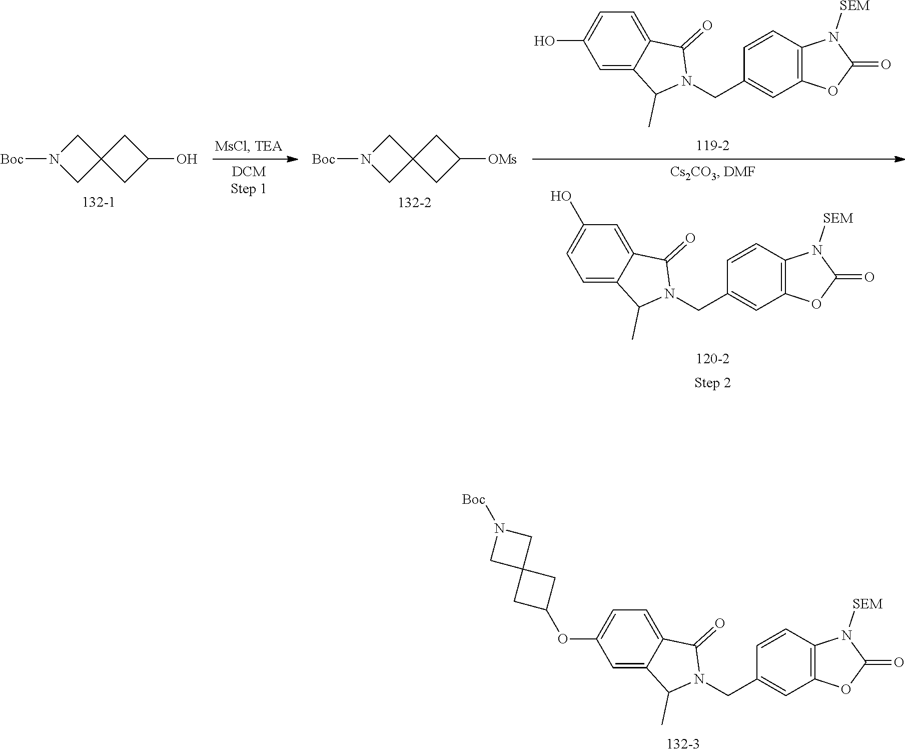 [SMILES: CC1c2cc(O)ccc2C(=O)N1Cc1ccc2c(c1)oc(=O)n2COCC[Si](C)(C)C ; CC(C)(C)OC(=O)N1CC2(CC(O)C2)C1 ; CC(C)(C)OC(=O)N1CC2(CC(OS(C)(=O)=O)C2)C1 ; CC1c2ccc(O)cc2C(=O)N1Cc1ccc2c(c1)oc(=O)n2COCC[Si](C)(C)C ; CC1c2cc(OC3CC4(C3)CN(C(=O)OC(C)(C)C)C4)ccc2C(=O)N1Cc1ccc2c(c1)oc(=O)n2COCC[Si](C)(C)C]

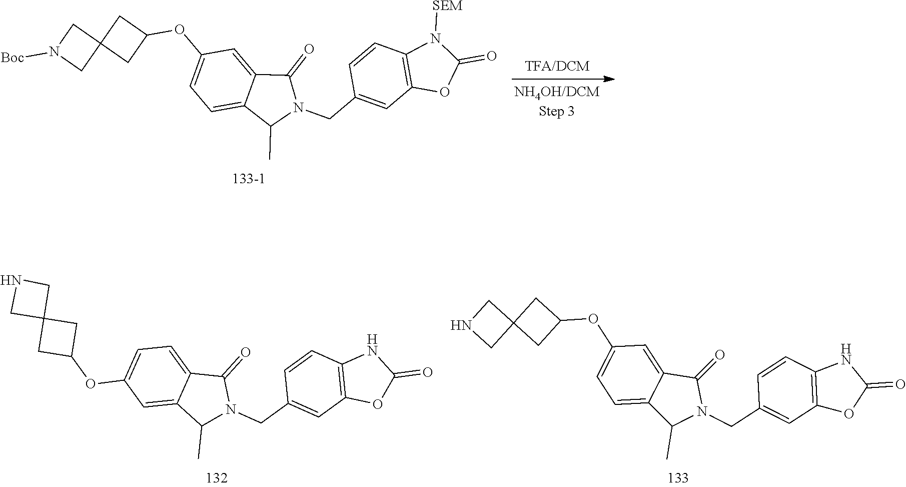 [SMILES: CC1c2ccc(OC3CC4(C3)CN(C(=O)OC(C)(C)C)C4)cc2C(=O)N1Cc1ccc2c(c1)oc(=O)n2COCC[Si](C)(C)C ; CC1c2cc(OC3CC4(CNC4)C3)ccc2C(=O)N1Cc1ccc2[nH]c(=O)oc2c1 ; CC1c2ccc(OC3CC4(CNC4)C3)cc2C(=O)N1Cc1ccc2[nH]c(=O)oc2c1]

### Step 1. tert-butyl 6-((methylsulfonyl)oxy)-2-azaspiro[3.3]heptane-2-carboxylate (132-2)

To a solution of tert-butyl 6-hydroxy-2-azaspiro[3.3]heptane-2-carboxylate (500 mg, 2.34 mmol) in DCM (10 mL) were added MsCl (324 mg, 2.82 mmol), TEA (474 mg, 4.69 mmol), and stirred at r.t. overnight. The mixture was diluted with DCM (30 mL) and washed with water (3×20 mL). The organic layer was separated, dried over anhydrous Na₂SO₄ and concentrated to afford the title compound (600 mg, 88.10%) as a yellow oil.

### Step 2. Mixture of tert-butyl 6-((3-methyl-1-oxo-2-((2-oxo-3-((2-(trimethylsilyl)ethoxy) methyl)-2,3-dihydrobenzo[d]oxazol-6-yl)methyl)isoindolin-5-yl)oxy)-2-azaspiro[3.3]heptane-2-carboxylate (132-3) and tert-butyl 6-((1-methyl-3-oxo-2-((2-oxo-3-((2-(trimethylsilyl)ethoxy)methyl)-2,3-dihydrobenzo[d]oxazol-6-yl)methyl)isoindolin-5-yl)oxy)-2-azaspiro[3.3]heptane-2-carboxylate (133-1)

To a solution of mixture of 119-2 and 120-2 (400 mg, 0.91 mmol) in DMF (10 mL) were added tert-butyl 6-((methylsulfonyl)oxy)-2-azaspiro[3.3]heptane-2-carboxylate (317 mg, 1.09 mmol) and Cs₂CO₃ (592 mg, 1.82 mmol) and stirred at 100° C. overnight. The reaction mixture was then quenched with water, and extracted with EA (3×50 mL). The combined organic phase was washed with brine, dried over anhydrous Na₂SO₄ and concentrated to afford a mixture 132-3 and 133-1 (200 mg, 34.7%) as a yellow oil. MS (ESI) m/z 636 [M+H]⁺.

### Step 3. 6-((5-((2-azaspiro[3.3]heptan-6-yl)oxy)-3-methyl-1-oxoisoindolin-2-yl)methyl) benzo[d]oxazol-2(3H)-one (132) and 6-((5-((2-azaspiro[3.3]heptan-6-yl)oxy)-1-methyl-3-oxoisoindolin-2-yl)methyl)benzo[d]oxazol-2(3H)-one (133)

To a solution of mixture of 132-3 and 132-1 (200 mg, 0.16 mmol) in DCM (10 mL) was added trifluoroacetic acid (4 mL). The mixture was stirred at r.t. for 2h, and then concentrated under vacuum. The residue was dissolved with DCM (4 mL), and NH₄OH (2 mL) was added dropwise. The mixture was stirred at r.t. for 1 h. The reaction mixture was concentrated, and the residue was purified by Prep-HPLC to afford two compounds: compound 132 and compound 133. One of the two compounds was a white solid (11 mg, 17%), with RT=2.07 min (HPLC), MS (ESI) m/z 406 [M+H]⁺. ¹H NMR (400 MHz, DMSO-d₆) δ 11.64 (s, 1H), 7.61 (d, J=8.4 Hz, 1H), 7.21 (s, 1H), 7.09-7.00 (m, 3H), 6.92 (d, J=8.4, 2.2 Hz, 1H), 4.97 (d, J=15.2 Hz, 1H), 4.72-4.65 (m, 1H), 4.42-4.30 (m, 2H), 4.04 (t, J=6.2 Hz, 2H), 3.96 (t, J=6.2 Hz, 2H), 2.87-2.77 (m, 2H), 2.34-2.21 (m, 2H), 1.37 (d, J=6.6 Hz, 3H). The other one of the two compounds was a white solid, (4 mg, 6%), with RT=2.53 min (HPLC), MS (ESI) m/z 406 [M+H]⁺. ¹H NMR (400 MHz, DMSO-d₆) δ 11.63 (s, 1H), 7.61 (d, J=9.0 Hz, 1H), 7.22 (s, 1H), 7.10-7.02 (m, 4H), 4.97 (d, J=15.2 Hz, 1H), 4.75-4.68 (m, 1H), 4.42-4.30 (m, 2H), 4.04 (t, J=6.2 Hz, 2H), 3.96 (t, J=6.2 Hz, 2H), 2.87-2.77 (m, 2H), 2.33-2.25 (m, 2H), 1.37 (d, J=6.6 Hz, 3H).

### 6-((1-oxo-3-(2-(trifluoromethyl) benzyl) isoindolin-2-yl)methyl)benzo[d]oxazol-2(3H)-one (134)

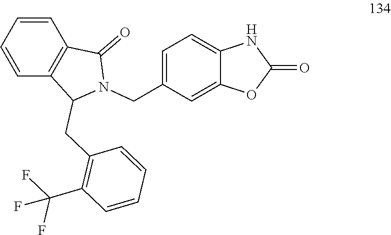 [SMILES: O=C1c2ccccc2C(Cc2ccccc2C(F)(F)F)N1Cc1ccc2[nH]c(=O)oc2c1]

The title compound 134 was prepared according to the procedure described for compound 124 as a white solid (141 mg, 32.6%). MS (ESI) m/z 439 [M+H]⁺. ¹H NMR (400 MHz, DMSO-d₆) δ 11.62 (s, 1H), 7.78-7.71 (m, 2H), 7.66 (t, J=7.6 Hz, 1H), 7.55-7.40 (m, 4H), 7.06-7.00 (m, 2H), 6.94 (dd, J=8.0, 1.6 Hz, 1H), 6.65 (d, J=7.5 Hz, 1H), 5.11 (d, J=15.3 Hz, 1H), 4.84 (t, J=7.2 Hz, 1H), 4.27 (d, J=15.3 Hz, 1H), 3.57 (dd, J=14.4, 6.4 Hz, 1H), 2.93 (dd, J=14.4, 8.0 Hz, 1H).

### (R)-6-((1-oxo-3-(2-(trifluoromethyl)benzyl)isoindolin-2-yl)methyl)benzo[d]oxazol-2(3H)-one (135) and (S)-6-((1-oxo-3-(2-(trifluoromethyl)soindolin-2-yl)methyl) benzo[d]oxazol-2(3H)-one (136)

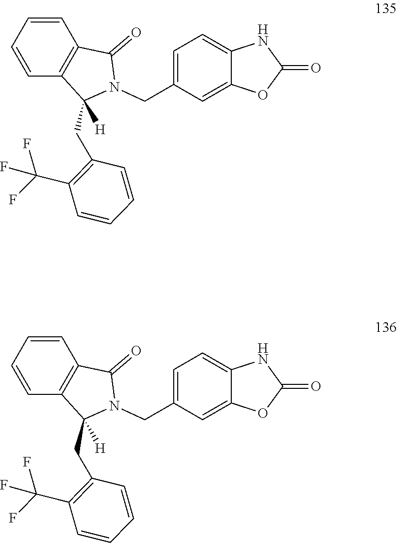 [SMILES: O=C1c2ccccc2[C@@H](Cc2ccccc2C(F)(F)F)N1Cc1ccc2[nH]c(=O)oc2c1 ; O=C1c2ccccc2[C@H](Cc2ccccc2C(F)(F)F)N1Cc1ccc2[nH]c(=O)oc2c1]

Chiral separation of compound 134 by chiral prep-HPLC (Column: CHIRALPAK IG; Column size: 2 cm×25 cm, 5 um; Mobile phase A: Hex; Mobile phase B: EtOH; Flow rate: 20 mL/min; Detector: 220 nm.) was conducted to afford two enantiomers: compound 135 and 136. One of the two enantiomers was a white solid, with RT=2.075 min (CHIRAL HPLC, column: CHIRALPAK IG-3; column size: 4.6*50 mm, 3 μm; mobile phase: Hex (0.1% DEA):EtOH=70:30; flow: 1.0 mL/min). MS (ESI) m/z 439 [M+H]⁺. ¹H NMR (400 MHz, DMSO-d₆) δ 11.61 (s, 1H), 7.76-7.72 (m, 2H), 7.66 (t, J=7.5 Hz, 1H), 7.55-7.40 (m, 4H), 7.05-7.00 (m, 2H), 6.94 (dd, J=8.0, 1.6 Hz, 11H), 6.64 (d, J=7.5 Hz, 1H), 5.10 (d, J=15.3 Hz, 1H), 4.83 (t, J=7.2 Hz, 11H), 4.26 (d, J=15.3 Hz, 1H), 3.57 (dd, J=14.4, 6.4 Hz, 1H), 2.92 (dd, J=14.4, 8.0 Hz, 1H). The other one of the two enantiomers was a white solid, with RT=2.971 min (CHIRAL HPLC, column: CHIRALPAK IG-3; column size: 4.6*50 mm, 3 μm; mobile phase: Hex (0.1% DEA):EtOH=70:30; flow: 1.0 mL/min). MS (ESI) m/z 439 [M+H]⁺. ¹H NMR (400 MHz, DMSO-d₆) δ 11.61 (s, 1H), 7.76-7.72 (m, 2H), 7.66 (t, J=7.5 Hz, 1H), 7.55-7.40 (m, 4H), 7.05-7.00 (m, 2H), 6.94 (dd, J=8.0, 1.5 Hz, 1H), 6.64 (d, J=7.5 Hz, 1H), 5.10 (d, J=15.3 Hz, 1H), 4.83 (t, J=7.2 Hz, 1H), 4.26 (d, J=15.3 Hz, 1H), 3.57 (dd, J=14.3, 6.4 Hz, 1H), 2.92 (dd, J=14.3, 8.0 Hz, 1H).

### 6-((1-oxo-3-(pyridin-3-ylmethyl)isoindolin-2-yl)methyl)benzo[d]oxazol-2(3H)-one (137)

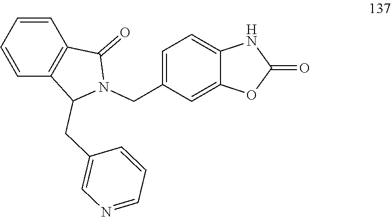 [SMILES: O=C1c2ccccc2C(Cc2cccnc2)N1Cc1ccc2[nH]c(=O)oc2c1]

The title compound 137 was prepared according to the procedure described for compound 124 as a white solid (28 mg, 27%). MS (ESI) m/z 372 [M+H]⁺. ¹H NMR (400 MHz, DMSO-d₆) δ 11.69 (s, 1H), 8.28 (dd, J=4.8, 1.6 Hz, 1H), 8.10 (d, J=2.1 Hz, 1H), 7.60-7.49 (m, 3H), 7.42 (t, J=7.3 Hz, 1H), 7.29-7.25 (m, 1H), 7.24 (s, 1H), 7.15-6.98 (m, 3H), 5.16 (d, J=15.1 Hz, 1H), 4.77 (t, J=4.6 Hz, 1H), 4.52 (d, J=15.1 Hz, 1H), 3.37 (d, J=4.8 Hz, 2H).

### (R)-6-((5-bromo-3-methyl-1-oxoisoindolin-2-yl)methyl)benzo[d]oxazol-2(3H)-one (138) and (S)-6-((5-bromo-3-methyl-1-oxoisoindolin-2-yl)methyl)benzo[d]oxazol-2(3H)-one (139)

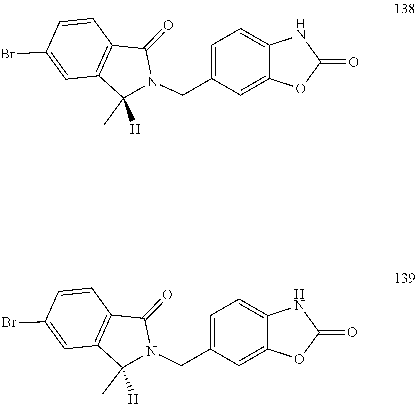 [SMILES: C[C@@H]1c2cc(Br)ccc2C(=O)N1Cc1ccc2[nH]c(=O)oc2c1 ; C[C@H]1c2cc(Br)ccc2C(=O)N1Cc1ccc2[nH]c(=O)oc2c1]

The title compound 138 and compound 139 were prepared, respectively, according to the procedure described for step 2 in general procedure E. The stereoisomer of Int 10 or Int 11 that has RT of 1.728 min was used as a starting material to afford one white solid (40 mg, 67.4%) that has the following characterization: MS (ESI) m/z 373 [M+H]⁺. ¹H NMR (400 MHz, DMSO-d₆) δ 11.62 (s, 1H), 7.89 (s, 1H), 7.72-7.62 (m, 2H), 7.25 (s, 1H), 7.12-7.01 (m, 2H), 4.99 (d, J=15.2 Hz, 1H), 4.50-4.36 (m, 2H), 1.40 (d, J=6.7 Hz, 3H). The stereoisomer of Int 10 or Int 11 that has RT of 1.969 min was used as a starting material to afford one white solid (40 mg, 67.4%) that has the following characterization: MS (ESI) m/z 373 [M+H]⁺. ¹H NMR (400 MHz, DMSO-d₆) δ 11.62 (s, 1H), 7.89 (s, 1H), 7.73-7.62 (m, 2H), 7.25 (s, 1H), 7.11-7.03 (m, 2H), 4.99 (d, J=15.2 Hz, 1H), 4.49-4.37 (m, 2H), 1.40 (d, J=6.7 Hz, 3H).

### 6-((1-oxo-3-(thiazol-2-ylmethyl) isoindolin-2-yl) methyl) benzo[d]oxazol-2(3H)-one (140)

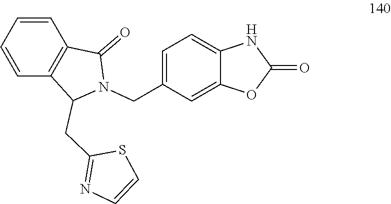 [SMILES: O=C1c2ccccc2C(Cc2nccs2)N1Cc1ccc2[nH]c(=O)oc2c1]

The title compound 140 was prepared according to the procedure described for compound 124 as a white solid (4 mg, 9%). MS (ESI) m/z 378 [M+H]⁺. ¹H NMR (400 MHz, DMSO-d₆) δ 11.61 (s, 1H), 7.67-7.54 (m, 3H), 7.53-7.40 (m, 3H), 7.26 (s, 1H), 7.12-7.01 (m, 2H), 5.15 (d, J=15.2 Hz, 1H), 4.85 (t, J=4.6 Hz, 1H), 4.44 (d, J=15.2 Hz, 1H), 3.86-3.72 (m, 2H).

### 6-((3-oxo-1-(2-(trifluoromethyl)benzyl)-1,3-dihydro-2H-indazol-2-yl)methyl)benzo[d]oxazol-2(3H)-one (141)

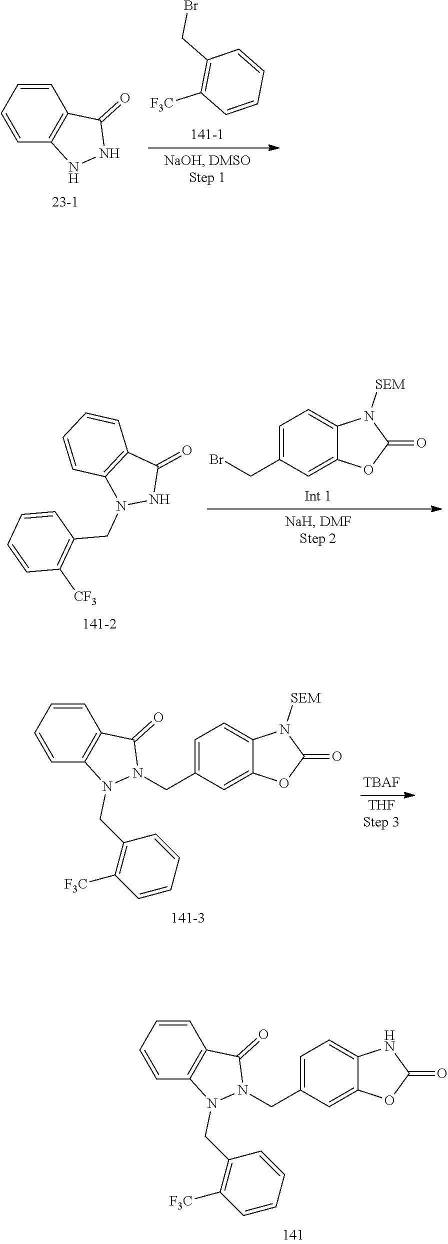

### Step 1. 1-(2-(trifluoromethyl)benzyl)-1,2-dihydro-3H-indazol-3-one (141-2)

To a solution of 1,2-dihydro-3H-indazol-3-one (200 mg, 1.49 mmol) in DMSO (2.5 mL) was added NaOH (119 mg, 2.98 mmol) and 1-(bromomethyl)-2-(trifluoromethyl) benzene (356 mg, 1.49 mmol) under N₂. The mixture was stirred for 2 h, and purified by C18 column (ACN=55%) to give 1-(2-(trifluoromethyl)benzyl)-1,2-dihydro-3H-indazol-3-one (170 mg, 39%) as a white solid. MS (ESI) m/z 293 [M+H]⁺.

### Step 2. 6-((3-oxo-1-(2-(trifluoromethyl)benzyl)-1,3-dihydro-2H-indazol-2-yl)methyl)-3-((2-(trimethylsilyl)ethoxy)methyl)benzo[d]oxazol-2(3H)-one (141-3)

To a solution of 1-(2-(trifluoromethyl)benzyl)-1,2-dihydro-3H-indazol-3-one (440 mg, 1.51 mmol) in DMF (5 mL) was added NaH (40 mg, 1.66 mmol) at 0° C. under N₂. The mixture was stirred for 30 min. 6-(bromomethyl)-3-((2-(trimethylsilyl)ethoxy)methyl)benzo[d]oxazol-2(3H)-one (701 mg, 1.96 mmol) was added. This suspension was allowed to stir for an additional 2 h. Water was added and then extracted with EtOAc. The organic layer was separated, dried over anhydrous Na₂SO₄ and concentrated under vacuum. The residue was purified by prep TLC (DCM/MeOH=30/1) to give 6-((3-oxo-1-(2-(trifluoromethyl)benzyl)-1,3-dihydro-2H-indazol-2-yl)methyl)-3-((2-(trimethylsilyl)ethoxy)methyl)benzo[d]oxazol-2(3H)-one (140 mg, 16%) as a yellow solid. MS (ESI) m/z 570 [M+H]⁺.

### Step 3. 6-((3-oxo-1-(2-(trifluoromethyl)benzyl)-1,3-dihydro-2H-indazol-2-yl)methyl)benzo[d]oxazol-2(3H)-one (141)

To a solution of 6-((3-oxo-1-(2-(trifluoromethyl)benzyl)-1,3-dihydro-2H-indazol-2-yl)methyl)-3-((2-(trimethylsilyl)ethoxy)methyl)benzo[d]oxazol-2(3H)-one (140 mg, 0.24 mmol) in THF (5 mL) was added TBAF (192 mg, 0.73 mmol) under N₂. The mixture was stirred at rt for 2 days, the mixture was diluted with EtOAc, and washed with water. The organic layer was separated, dried over anhydrous Na₂SO₄ and concentrated under vacuum. The residue was purified by prep HPLC to give 6-((3-oxo-1-(2-(trifluoromethyl)benzyl)-1,3-dihydro-2H-indazol-2-yl)methyl) benzo[d]oxazol-2(3H)-one (9 mg, 8.3%) as a light yellow solid. MS (ESI) m/z 440 [M+H]⁺. ¹H NMR (400 MHz, DMSO-d₆) δ 11.60 (s, 1H), 7.81-7.74 (m, 2H), 7.63-7.52 (m, 3H), 7.29-7.22 (m, 1H), 7.06 (s, 1H), 7.04-6.95 (m, 3H), 6.76 (t, J=8.0 Hz, 1H), 6.70 (d, J=8.0 Hz, 1H), 6.04 (d, J=2.6 Hz, 1H), 5.12 (d, J=15.4 Hz, 11H), 3.77 (d, J=15.4 Hz, 1H).

### 6-((1-(2-methoxybenzyl)-3-oxoisoindolin-2-yl)methyl)benzo[d]oxazol-2(3H)-one (142)

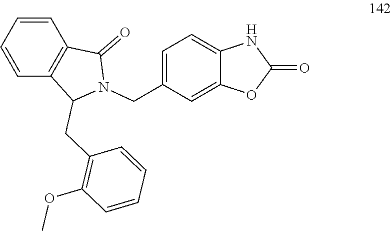 [SMILES: COc1ccccc1CC1c2ccccc2C(=O)N1Cc1ccc2[nH]c(=O)oc2c1]

The title compound 142 was prepared according to the procedure described for compound 124 as a white solid (12 mg, 5.9%). MS (ESI) m/z 401 [M+H]⁺. ¹H NMR (400 MHz, DMSO-d₆) δ 11.63 (s, 1H), 7.71-7.62 (m, 1H), 7.51-7.40 (m, 2H), 7.28-7.22 (m, 1H), 7.09 (d, J=1.5 Hz, 1H), 7.05 (d, J=7.9 Hz, 1H), 7.02-6.91 (m, 4H), 6.84 (t, J=7.6 Hz, 1H), 5.17 (d, J=15.1 Hz, 1H), 4.68 (dd, J=7.6, 5.1 Hz, 1H), 4.35 (d, J=15.1 Hz, 1H), 3.67 (s, 3H), 3.43 (dd, J=13.4, 5.1 Hz, 1H), 2.81 (dd, J=13.5, 7.6 Hz, 1H).

### 6-((3-methyl-5-(morpholinomethyl)-1-oxoisoindolin-2-yl)methyl)benzo[d]oxazol-2(3H)-one (143)

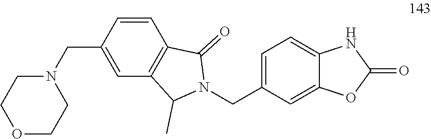 [SMILES: CC1c2cc(CN3CCOCC3)ccc2C(=O)N1Cc1ccc2[nH]c(=O)oc2c1]

The title compound 143 was prepared according to the procedure described for compound 96 as a white solid (9 mg, 23%). MS (ESI) m/z 394 [M+H]⁺. ¹H NMR (400 MHz, DMSO-d₆) δ 11.68 (s, 1H), 7.66 (d, J=7.7 Hz, 1H), 7.50 (s, 1H), 7.44 (dd, J=7.8, 1.4 Hz, 1H), 7.24 (d, J=1.4 Hz, 1H), 7.09 (dd, J=8.0, 1.6 Hz, 1H), 7.04 (d, J=8.0 Hz, 1H), 5.01 (d, J=15.2 Hz, 1H), 4.46-4.36 (m, 2H), 3.61-3.49 (m, 6H), 2.35 (t, J=4.7 Hz, 4H), 1.39 (d, J=6.7 Hz, 3H).

### (R)-6-((3-methyl-5-(morpholinomethyl)-1-oxoisoindolin-2-yl)methyl)benzo[d]oxazol-2(3H)-one (144) and (S)-6-((3-methyl-5-(morpholinomethyl)-1-oxoisoindolin-2-yl)methyl)benzo[d]oxazol-2(3H)-one (145)

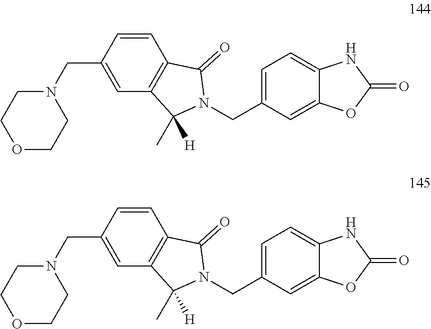 [SMILES: C[C@@H]1c2cc(CN3CCOCC3)ccc2C(=O)N1Cc1ccc2[nH]c(=O)oc2c1 ; C[C@H]1c2cc(CN3CCOCC3)ccc2C(=O)N1Cc1ccc2[nH]c(=O)oc2c1]

The title compound 144 and compound 145 were prepared, respectively, according to the procedure described for compound 96. The stereoisomer of Int 10 or Int 11 that has RT of 1.728 min was used as a starting material to afford one white solid (69 mg, 44%) that has the following characterization: MS (ESI) m/z 394 [M+H]⁺. ¹H NMR (400 MHz, DMSO-d₆) δ 11.63 (s, 1H), 7.71 (d, J=7.7 Hz, 1H), 7.55 (s, 1H), 7.49 (d, J=7.8 Hz, 1H), 7.24 (d, J=1.5 Hz, 1H), 7.10 (dd, J=8.1, 1.5 Hz, 1H), 7.04 (d, J=8.0 Hz, 1H), 5.02 (d, J=15.2 Hz, 1H), 4.48-4.37 (m, 2H), 3.75-3.52 (m, 6H), 2.63-2.40 (m, 4H), 1.40 (d, J=6.7 Hz, 3H). The stereoisomer of Int 10 or Int 11 that has RT of 1.969 min was used as a starting material to afford one white solid (56 mg, 36%) that has the following characterization: MS (ESI) m/z 394 [M+H]⁺. ¹H NMR (400 MHz, DMSO-d₆) δ 11.64 (s, 1H), 7.72 (d, J=7.7 Hz, 1H), 7.57 (s, 1H), 7.50 (d, J=7.8 Hz, 1H), 7.24 (d, J=1.5 Hz, 1H), 7.10 (d, J=8.1 Hz, 1H), 7.05 (d, J=7.9 Hz, 1H), 5.02 (d, J=15.2 Hz, 1H), 4.49-4.37 (m, 2H), 3.75-3.52 (m, 6H), 2.63-2.40 (m, 4H), 1.40 (d, J=6.7 Hz, 3H).

### 2-((3-oxo-2-((2-oxo-2,3-dihydrobenzo[d]oxazol-6-yl)methyl)isoindolin-1-yl)methyl) benzonitrile (147)

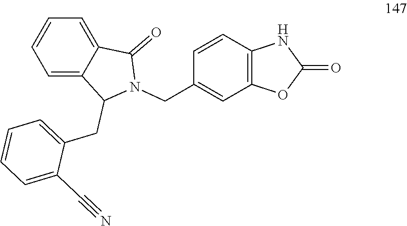 [SMILES: N#Cc1ccccc1CC1c2ccccc2C(=O)N1Cc1ccc2[nH]c(=O)oc2c1]

The title compound 147 was prepared according to general procedure E as a white solid (9 mg, 7.3%). MS (ESI) m/z 396 [M+H]⁺. ¹H NMR (400 MHz, DMSO-d₆) δ 11.62 (s, 1H), 7.72 (dd, J=7.7, 1.4 Hz, 1H), 7.65 (d, J=7.6 Hz, 1H), 7.60-7.56 (m, 1H), 7.53-7.49 (m, 1H), 7.47-7.39 (m, 2H), 7.28 (d, J=7.6 Hz, 1H), 7.14 (s, 1H), 7.12 (d, J=7.6 Hz, 1H), 7.04-7.00 (m, 2H), 5.15 (d, J=15.2 Hz, 1H), 4.87 (t, J=5.8 Hz, 1H), 4.47 (d, J=15.2 Hz, 1H), 3.60 (dd, J=14.4, 5.0 Hz, 1H), 3.28 (dd, J=14.4, 6.4 Hz, 1H).

### 4-((3-oxo-2-((2-oxo-2,3-dihydrobenzo[d]oxazol-6-yl)methyl)isoindolin-1-yl)methyl) benzonitrile (148)

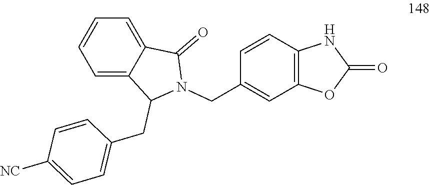 [SMILES: N#Cc1ccc(CC2c3ccccc3C(=O)N2Cc2ccc3[nH]c(=O)oc3c2)cc1]

The title compound 148 was prepared according to general procedure E as a white solid (50 mg, 53%). MS (ESI) m/z 396 [M+H]⁺. ¹H NMR (400 MHz, DMSO-d₆) δ 11.63 (s, 1H), 7.65-7.51 (m, 4H), 7.47-7.39 (m, 2H), 7.22 (d, J=1.5 Hz, 11H), 7.19-7.1 I (m, 2H), 7.11-7.01 (m, 2H), 5.14 (d, J=15.1 Hz, 1H), 4.79 (t, J=4.9 Hz, 1H), 4.49 (d, J=15.1 Hz, 1H), 3.46 (dd, J=14.1, 4.2 Hz, 1H), 3.37 (dd, J=14.0, 5.9 Hz, 1H).

### 6-((5-((2-oxaspiro[3.3]heptan-6-yl)amino)-3-methyl-1-oxoisoindolin-2-yl)methyl)benzo[d]oxazol-2(3H)-one (149)

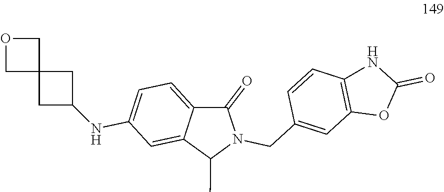 [SMILES: CC1c2cc(NC3CC4(COC4)C3)ccc2C(=O)N1Cc1ccc2[nH]c(=O)oc2c1]

The title compound 149 was prepared according to the procedure described for compound 109 as a white solid (7 mg, 7.4%). MS (ESI) m/z 406 [M+H]⁺. ¹H NMR (400 MHz, DMSO-d₆) δ 11.63 (s, 1H), 7.37 (d, J=8.3 Hz, 1H), 7.15 (s, 1H), 7.03-6.99 (m, 2H), 6.58-6.52 (m, 2H), 6.49 (d, J=1.9 Hz, 1H), 4.93 (d, J=15.2 Hz, 1H), 4.62 (s, 2H), 4.49 (s, 2H), 4.29 (d, J=15.2 Hz, 1H), 4.22 (q, J=6.6 Hz, 1H), 3.75-3.63 (m, 1H), 2.73-2.61 (m, 2H), 2.05-1.93 (m, 2H), 1.31 (d, J=6.6 Hz, 3H).

### 6-((1-(oxazol-2-ylmethyl)-3-oxoisoindolin-2-yl)methyl)benzo[d]oxazol-2(3H)-one (150)

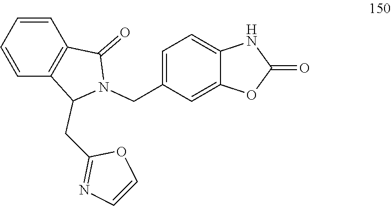 [SMILES: O=C1c2ccccc2C(Cc2ncco2)N1Cc1ccc2[nH]c(=O)oc2c1]

The title compound 150 was prepared according to general procedure E as a white solid (81 mg, 26.9%). MS (ESI) m/z 362 [M+H]⁺. ¹H NMR (400 MHz, DMSO-d₆) δ 11.62 (s, 1H), 7.88 (d, J=0.9 Hz, 1H), 7.66 (d, J=7.5 Hz, 1H), 7.59-7.54 (m, 1H), 7.47 (t, J=7.5 Hz, 1H), 7.40 (d, J=7.3 Hz, 1H), 7.23 (d, J=1.4 Hz, 1H), 7.11-6.98 (m, 3H), 5.13 (d, J=15.3 Hz, 1H), 4.81 (dd, J=6.2, 4.2 Hz, 1H), 4.40 (d, J=15.3 Hz, 1H), 3.57 (dd, J=15.7, 4.2 Hz, 1H), 3.42 (dd, J=15.6, 6.3 Hz, 1H).

### 6-((7-(2-methylbenzyl)-5-oxo-5,7-dihydro-6H-pyrrolo[3,4-b]pyridin-6-yl)methyl)benzo[d]oxazol-2(3H)-one (151)

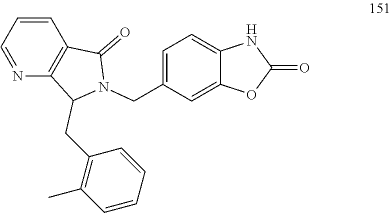 [SMILES: Cc1ccccc1CC1c2ncccc2C(=O)N1Cc1ccc2[nH]c(=O)oc2c1]

The title compound 151 was prepared according to general procedure E using 6-((5-oxo-5,7-dihydro-6H-pyrrolo[3,4-b]pyridin-6-yl)methyl)-3-((2-(trimethylsilyl)ethoxy)methyl)benzo[d]oxazol-2(3H)-one (preparation according to the procedure outlined for Int 6) as a starting material to afford a white solid (97 mg, 64.9%). MS (ESI) m/z 386 [M+H]⁺. ¹H NMR (400 MHz, DMSO-d₆) δ 11.60 (s, 1H), 8.72 (dd, J=4.9, 1.6 Hz, 1H), 8.08 (dd, J=7.6, 1.6 Hz, 1H), 7.50 (dd, J=7.7, 4.9 Hz, 1H), 7.15-7.01 (m, 4H), 6.97 (d, J=7.9 Hz, 1H), 6.88 (d, J=1.5 Hz, 1H), 6.82 (dd, J=8.0, 1.6 Hz, 1H), 5.17 (d, J=15.0 Hz, 1H), 4.73 (dd, J=6.6, 4.8 Hz, 1H), 4.22 (d, J=15.2 Hz, 1H), 3.37 (dd, J=14.6, 4.9 Hz, 1H), 3.18 (dd, J=14.6, 6.6 Hz, 1H), 2.18 (s, 3H).

### Mixture of 6-(((R)-1-oxo-3-((R)-1-(pyridin-2-yl)ethyl)isoindolin-2-yl)methyl)benzo[d]oxazol-2(3H)-one (152) and 6-(((S)-1-oxo-3-((S)-1-(pyridin-2-yl)ethyl)isoindolin-2-yl)methyl)benzo[d]oxazol-2(3H)-one (153), and mixture of 6-(((S)-1-oxo-3-((R)-1-(pyridin-2-yl)ethyl)isoindolin-2-yl)methyl)benzo[d]oxazol-2(3H)-one (154) and 6-(((R)-1-oxo-3-((S)-1-(pyridin-2-yl)ethyl)isoindolin-2-yl)methyl)benzo[d]oxazol-2(3H)-one (155)

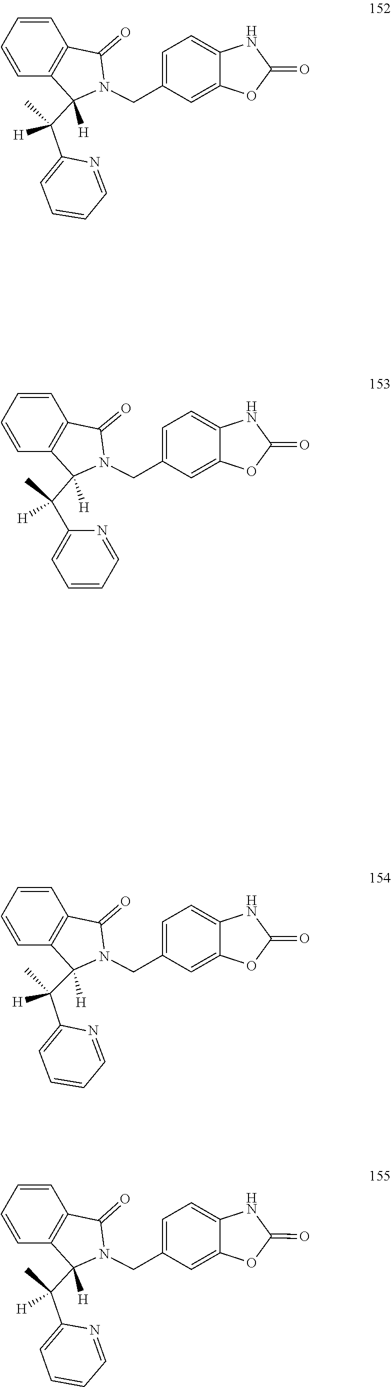 [SMILES: C[C@@H](c1ccccn1)[C@@H]1c2ccccc2C(=O)N1Cc1ccc2[nH]c(=O)oc2c1 ; C[C@H](c1ccccn1)[C@H]1c2ccccc2C(=O)N1Cc1ccc2[nH]c(=O)oc2c1 ; C[C@@H](c1ccccn1)[C@H]1c2ccccc2C(=O)N1Cc1ccc2[nH]c(=O)oc2c1 ; C[C@H](c1ccccn1)[C@@H]1c2ccccc2C(=O)N1Cc1ccc2[nH]c(=O)oc2c1]

A mixture of 152 and 153 and a mixture of 154 and 155 were prepared, respectively, according to general procedure E. After Pre-HPLC separation, 2 diastereoisomers (each one contains two enantiomers) were obtained: mixture of 152 and 153, and mixture of 154 and 155. One of the two diastereoisomers was a white solid (45 mg, 22%), with RT=3.24 min (HPLC), MS (ESI) m/z 386 [M+H]⁺. ¹H NMR (400 MHz, DMSO-d₆) δ 11.57 (s, 1H), 8.49-8.47 (m, 1H), 7.75-7.71 (m, 2H), 7.68-7.59 (m, 2H), 7.50 (t, J=7.4 Hz, 1H), 7.32-7.29 (m, 1H), 7.24 (d, J=7.9 Hz, 1H), 6.91 (d, J=8.3 Hz, 1H), 6.58-6.55 (m, 2H), 5.07 (d, J=15.2 Hz, 1H), 4.97 (d, J=2.6 Hz, 1H), 3.95 (d, J=15.2 Hz, 1H), 3.83-3.80 (m, 1H), 1.13 (d, J=7.2 Hz, 3H). The other one of the two diastereoisomers was a white solid (40 mg, 20%), with RT=3.68 min (HPLC), MS (ESI) m/z 386 [M+H]⁺. ¹H NMR (400 MHz, DMSO-d₆) δ 11.64 (s, 1H), 8.63-8.61 (m, 1H), 7.84-7.80 (m, 1H), 7.73 (d, J=7.5 Hz, 1H), 7.44 (t, J=7.4 Hz, 1H), 7.36-7.31 (m, 3H), 7.28 (d, J=8.0 Hz, 1H), 7.15 (dd, J=8.0, 1.5 Hz, 1H), 7.07 (d, J=7.9 Hz, 1H), 6.19 (d, J=7.7 Hz, 1H), 5.21 (d, J=15.2 Hz, 1H), 5.13 (d, J=3.9 Hz, 1H), 4.48 (d, J=15.2 Hz, 1H), 3.93-3.81 (m, 1H), 0.73 (d, J=7.0 Hz, 3H).

### 6-((1-((4-methylpyridin-2-yl)methyl)-3-oxoisoindolin-2-yl)methyl)benzo[d]oxazol-2(3H)-one (156)

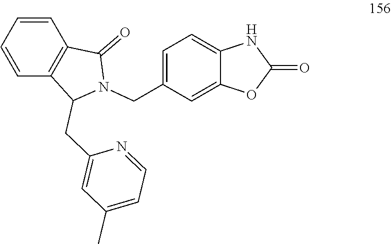 [SMILES: Cc1ccnc(CC2c3ccccc3C(=O)N2Cc2ccc3[nH]c(=O)oc3c2)c1]

The title compound 156 was prepared according to general procedure E as a white solid (85 mg, 42.5%). MS (ESI) m/z 386 [M+H]⁺. ¹H NMR (400 MHz, DMSO-d₆) δ 11.60 (s, 1H), 8.33 (d, J=5.0 Hz, 1H), 7.67 (d, J=6.9 Hz, 1H), 7.55-7.42 (m, 2H), 7.12 (d, J=7.4 Hz, 1H), 7.07-7.04 (m, 2H), 7.02 (d, J=7.9 Hz, 1H), 6.95 (dd, J=8.0, 1.5 Hz, 1H), 6.84 (s, 1H), 5.04 (d, J=15.3 Hz, 1H), 4.98 (t, J=6.1 Hz, 1H), 4.40 (d, J=15.3 Hz, 1H), 3.41 (dd, J=14.2, 5.4 Hz, 1H), 3.10 (dd, J=14.1, 6.9 Hz, 1H), 2.18 (s, 3H).

### 6-((1-(2-chlorobenzyl)-3-oxoisoindolin-2-yl)methyl)benzo[d]oxazol-2(3H)-one (157)

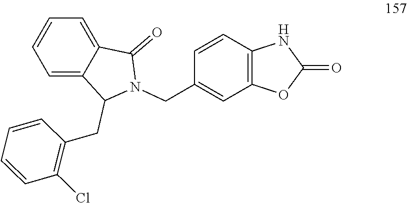 [SMILES: O=C1c2ccccc2C(Cc2ccccc2Cl)N1Cc1ccc2[nH]c(=O)oc2c1]

The title compound 157 was prepared according to general procedure E as a white solid (62 mg, 58.6%). MS (ESI) m/z 405 [M+H]⁺. ¹H NMR (400 MHz, DMSO-d₆) δ 11.59 (s, 1H), 7.73-7.67 (m, 1H), 7.51-7.40 (m, 3H), 7.35-7.19 (m, 3H), 7.08 (d, J=1.5 Hz, 1H), 7.05-6.95 (m, 2H), 6.93-6.87 (m, 1H), 5.16 (d, J=15.2 Hz, 1H), 4.77 (dd, J=7.6, 5.6 Hz, 1H), 4.39 (d, J=15.2 Hz, 1H), 3.55 (dd, J=13.8, 5.6 Hz, 1H), 2.98 (dd, J=13.8, 7.6 Hz, 1H).

### 6-((1-((6-methylpyridin-2-yl)methyl)-3-oxoisoindolin-2-yl)methyl)benzo[d]oxazol-2(3H)-one (158)

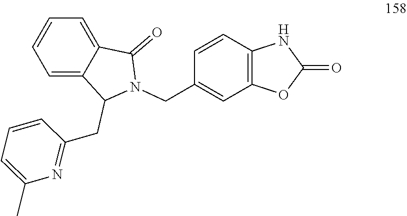 [SMILES: Cc1cccc(CC2c3ccccc3C(=O)N2Cc2ccc3[nH]c(=O)oc3c2)n1]

The title compound 158 was prepared according to general procedure E as a white solid (89 mg, 67%). MS (ESI) m/z 386 [M+H]⁺. ¹H NMR (400 MHz, DMSO-d₆) δ 11.64 (s, 1H), 7.77 (s, 1H), 7.68 (d, J=7.4 Hz, 1H), 7.59-7.44 (m, 2H), 7.35 (s, 1H), 7.25 (d, J=7.6 Hz, 1H), 7.08-7.01 (m, 3H), 6.95 (dd, J=8.0, 1.6 Hz, 1H), 5.12-4.96 (m, 2H), 4.42 (d, J=15.3 Hz, 1H), 3.50 (dd, J=14.4, 5.5 Hz, 11H), 3.39-3.26 (m, 1H), 2.46 (s, 3H).

### 6-((1-((3-chloropyridin-2-yl)methyl)-3-oxoisoindolin-2-yl)methyl)benzo[d]oxazol-2(3H)-one (159)

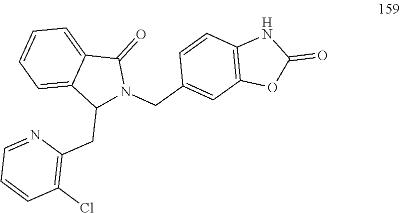 [SMILES: O=C1c2ccccc2C(Cc2ncccc2Cl)N1Cc1ccc2[nH]c(=O)oc2c1]

The title compound 159 was prepared according to general procedure E as a white solid (7 mg, 6.2%). MS (ESI) m/z 406 [M+H]⁺. ¹H NMR (400 MHz, DMSO-do) δ 11.60 (s, 1H), 8.50 (dd, J=4.7, 1.5 Hz, 1H), 7.86 (dd, J=8.1, 1.5 Hz, 1H), 7.76-7.69 (m, 1H), 7.52-7.44 (m, 2H), 7.36 (dd, J=8.1, 4.7 Hz, 1H), 7.09-7.02 (m, 1H), 7.03-6.95 (m, 2H), 6.91 (dd, J=8.0, 1.6 Hz, 1H), 5.13 (t, J=6.4 Hz, 1H), 5.04 (d, J=15.4 Hz, 1H), 4.45 (d, J=15.4 Hz, 1H), 3.59 (dd, J=15.0, 5.8 Hz, 1H), 3.20 (dd, J=15.1, 7.2 Hz, 1H).

### 6-((5-(2-methylbenzyl)-7-oxo-5,7-dihydro-6H-pyrrolo[3,4-b]pyridin-6-yl)methyl)benzo[d]oxazol-2(3H)-one (160)

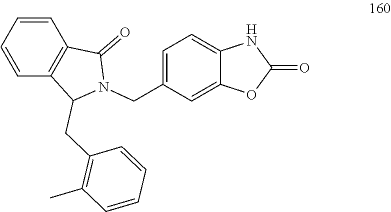 [SMILES: Cc1ccccc1CC1c2ccccc2C(=O)N1Cc1ccc2[nH]c(=O)oc2c1]

The title compound 160 was prepared according to general procedure E using 6-((7-oxo-5,7-dihydro-6H-pyrrolo[3,4-b]pyridin-6-yl)methyl)-3-((2-(trimethylsilyl)ethoxy)methyl)benzo[d]oxazol-2(3H)-one (preparation according to the procedure outlined for Int 6) as a starting material to afford a white solid (16 mg, 53%). MS (ESI) m/z 386 [M+H]⁺. ¹H NMR (400 MHz, DMSO-d₆) δ 11.64 (s, 1H), 8.69 (dd, J=4.9, 1.5 Hz, 1H), 7.41 (dd, J=7.8, 4.8 Hz, 1H), 7.24-7.00 (m, 8H), 5.19 (d, J=15.1 Hz, 1H), 4.70 (dd, J=8.7, 5.8 Hz, 1H), 4.45 (d, J=15.2 Hz, 1H), 3.50 (dd, J=13.8, 5.8 Hz, 1H), 2.75 (dd, J=13.8, 8.7 Hz, 1H), 2.09 (s, 3H).

### 6-((1-((5-methylpyridin-2-yl)methyl)-3-oxoisoindolin-2-yl)methyl)benzo[d]oxazol-2(3H)-one (161)

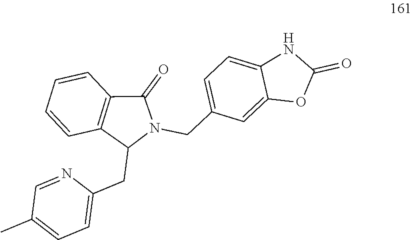 [SMILES: Cc1ccc(CC2c3ccccc3C(=O)N2Cc2ccc3[nH]c(=O)oc3c2)nc1]

The title compound 161 was prepared according to general procedure E as a white solid (90 mg, 58%). MS (ESI) m/z 386 [M+H]⁺. ¹H NMR (400 MHz, DMSO-d₆) δ 11.62 (s, 1H), 8.31 (d, J=2.2 Hz, 1H), 7.68-7.61 (m, 1H), 7.54-7.41 (m, 3H), 7.18-7.10 (m, 2H), 7.06-6.96 (m, 2H), 6.91 (d, J=7.9 Hz, 1H), 5.10 (d, J=15.3 Hz, 1H), 4.92 (dd, J=6.9, 4.8 Hz, 1H), 4.44 (d, J=15.3 Hz, 1H), 3.50-3.45 (m, 1H), 3.16 (dd, J=14.1, 7.0 Hz, 1H), 2.24 (s, 3H).

### rel-(S)-6-((3-methyl-5-(oxazol-5-yl)-1-oxoisoindolin-2-yl)methyl)benzo[d]oxazol-2(3H)-one (162)

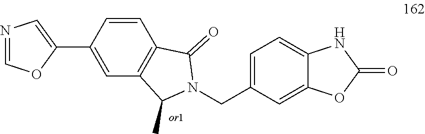 [SMILES: C[C@H]1c2cc(-c3cnco3)ccc2C(=O)N1Cc1ccc2[nH]c(=O)oc2c1]

The title compound 162 was prepared according to the procedure described for compound 61 using the stereoisomer of Int 10 or Int 11 that has RT of 1.969 min as a starting material to afford a white solid (5 mg, 15%) MS (ESI) m/z 362 [M+H]⁺. ¹H NMR (400 MHz, CD₃OD) δ 8.21-8.18 (m, 2H), 8.05 (d, J=0.9 Hz, 1H), 7.94 (d, J=8.3 Hz, 1H), 7.36 (d, J=0.8 Hz, 1H), 7.25 (s, 1H), 7.20-7.17 (m, 1H), 7.07 (d, J=8.0 Hz, 1H), 5.18 (d, J=15.2 Hz, 1H), 4.53 (d, J=15.3 Hz, 1H), 4.29 (t, J=6.6 Hz, 1H), 1.53 (d, J=6.8 Hz, 3H).

### rel-(S)-6-((3,5-dimethyl-1-oxoisoindolin-2-yl)methyl)benzo[d]oxazol-2(3H)-one (163)

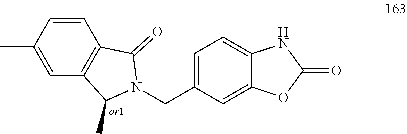 [SMILES: Cc1ccc2c(c1)[C@H](C)N(Cc1ccc3[nH]c(=O)oc3c1)C2=O]

The title compound 163 was prepared according to the procedure described for compound 125 using the stereoisomer of Int 10 or Int 11 that has RT of 1.969 min as a starting material to afford a white solid (14 mg, 33%). MS (ESI) m/z 309 [M+H]⁺. ¹H NMR (400 MHz, CD₃OD) δ 7.57 (d, J=8.2 Hz, 1H), 7.21 (d, J=7.7 Hz, 2H), 7.08 (s, 1H), 7.04 (d, J=8.0 Hz, 1H), 6.93 (d, J=8.0 Hz, 1H), 5.02 (d, J=15.3 Hz, 1H), 4.38-4.31 (m, 2H), 2.34 (s, 3H), 1.33 (d, J=6.7 Hz, 3H).

### 4-methyl-2-((3-oxo-2-((2-oxo-2,3-dihydrobenzo[d]oxazol-6-yl)methyl)isoindolin-1-yl) methyl)benzonitrile (164)

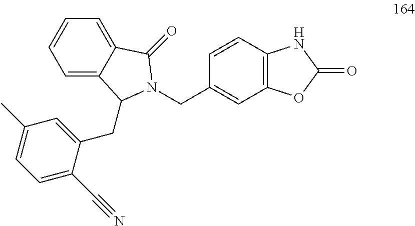 [SMILES: Cc1ccc(C#N)c(CC2c3ccccc3C(=O)N2Cc2ccc3[nH]c(=O)oc3c2)c1]

The title compound 164 was prepared according to general procedure E as an off-white solid (27 mg, 43%). MS (ESI) m/z 410 [M+H]⁺. ¹H NMR (400 MHz, DMSO-d(,) δ 11.66 (s, 1H), 7.73 (dd, J=6.0, 2.8 Hz, 1H), 7.62 (dd, J=7.8, 1.8 Hz, 1H), 7.53 (d, J=1.8 Hz, 1H), 7.52-7.44 (m, 2H), 7.36 (d, J=7.9 Hz, 1H), 7.05 (d, J=1.5 Hz, 1H), 7.00 (d, J=7.9 Hz, 1H), 6.95 (dd, J=8.0, 1.6 Hz, 1H), 6.87 (dd, J=5.9, 2.8 Hz, 1H), 5.09 (d, J=15.2 Hz, 1H), 4.78 (dd, J=7.6, 6.1 Hz, 1H), 4.36 (d, J=15.2 Hz, 1H), 3.43 (dd, J=14.1, 6.1 Hz, 1H), 2.90 (dd, J=14.1, 7.6 Hz, 1H), 2.17 (s, 3H).

The intermediate 164-3 was prepared as follows:

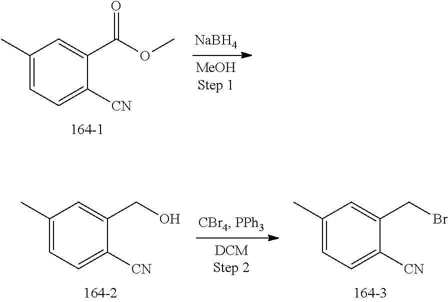

### Step 1. 2-(hydroxymethyl)-4-methylbenzonitrile (164-2)

To a solution of methyl 2-cyano-5-methylbenzoate (200 mg, 1.14 mmol) in methanol (5 mL) was added NaBH₄ (173 mg, 4.57 mmol) at 0° C. under N₂. The mixture was stirred for 2 h and concentrated under vacuum. The residue was purified by prep TLC (DCM/MeOH=30/1) to give 2-(hydroxymethyl)-4-methylbenzonitrile (154 mg, 91%) as a colorless semi-solid. MS (ESI) m/z 148 [M+H]⁺.

### Step 2. 2-(bromomethyl)-4-methylbenzonitrile (164-3)

To a solution of 2-(hydroxymethyl)-4-methylbenzonitrile (154 mg, 1.05 mmol) and PPh₃ (549 mg, 2.09 mmol) in DCM (3 mL) was added CBr₄ (347 mg, 1.05 mmol) at 0° C. under N₂. The mixture was stirred for 20 min and then allowed to warm to r.t. This suspension was allowed to stir for an additional 1 h. The solvent was removed in vacuo and the residue was purified by prep TLC (PE/EtOAc=5/1) to give 2-(bromomethyl)-4-methylbenzonitrile (70 mg, 31%) as a white solid. MS (ESI) m/z 210 [M+H]⁺.

### 6-((1-((2-methylpyridin-3-yl)methyl)-3-oxoisoindolin-2-yl)methyl)benzo[d]oxazol-2(3H)-one (165)

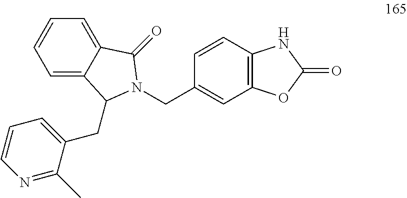 [SMILES: Cc1ncccc1CC1c2ccccc2C(=O)N1Cc1ccc2[nH]c(=O)oc2c1]

The title compound 165 was prepared according to general procedure E as an off-white solid (25 mg, 24%). MS (EST) m/z 386 [M+H]⁺. ¹H NMR (400 MHz, DMSO-d₆) δ 11.66 (s, 1H), 8.46 (d, J=5.2 Hz, 1H), 7.72 (d, J=7.1 Hz, 2H), 7.53-7.46 (m, 2H), 7.41 (d, J=6.3 Hz, 1H), 7.13 (s, 1H), 7.05-6.97 (m, 3H), 5.11 (d, J=15.2 Hz, 1H), 4.79 (t, J=6.5 Hz, 1H), 4.45 (d, J=15.2 Hz, 1H), 3.52 (dd, J=14.9, 5.3 Hz, 1H), 3.01 (dd, J=14.5, 7.6 Hz, 1H), 2.37 (s, 3H).

### 6-((1-(2-ethoxybenzyl)-3-oxoisoindolin-2-yl)methyl)benzo[d]oxazol-2(3H)-one (166)

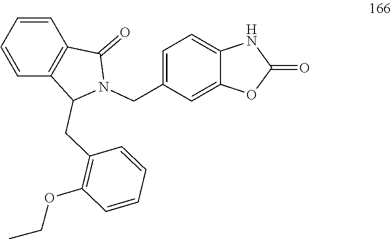 [SMILES: CCOc1ccccc1CC1c2ccccc2C(=O)N1Cc1ccc2[nH]c(=O)oc2c1]

The title compound 166 was prepared according to general procedure E as a white solid (5 mg, 15%). MS (ESI) m/z 415 [M+H]⁺. ¹H NMR (400 MHz, DMSO-d₆) δ 11.64 (s, 1H), 7.71-7.64 (m, 1H), 7.50-7.40 (m, 2H), 7.24 (td, J=7.8, 1.8 Hz, 1H), 7.06-7.01 (m, 2H), 7.00-6.91 (m, 3H), 6.88-6.79 (m, 2H), 5.16 (d, J=15.2 Hz, 1H), 4.76 (dd, J=7.8, 5.4 Hz, 1H), 4.37 (d, J=15.2 Hz, 1H), 3.98-3.83 (m, 2H), 3.43 (dd, J=13.3, 5.5 Hz, 1H), 2.75 (dd, J=13.3, 7.8 Hz, 1H), 1.11 (t, J=6.9 Hz, 3H).

### 6-((1-(cyclohexylmethyl)-3-oxoisoindolin-2-yl)methyl)benzo[d]oxazol-2(3H)-one (167)

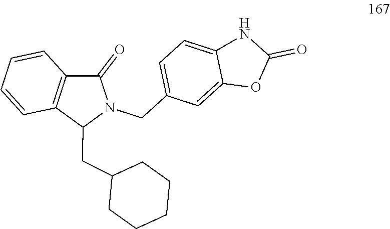 [SMILES: O=C1c2ccccc2C(CC2CCCCC2)N1Cc1ccc2[nH]c(=O)oc2c1]

The title compound 167 was prepared according to the procedure described for compound 67 as a white solid (15 mg, 48%). MS (ESI) m/z 377 [M+H]⁺. ¹H NMR (400 MHz, DMSO-d₆) δ 11.67 (s, 1H), 7.73 (d, J=7.5 Hz, 1H), 7.63-7.45 (m, 3H), 7.23 (s, 1H), 7.10-7.02 (m, 2H), 5.06 (d, J=15.2 Hz, 1H), 4.48 (dd, J=6.8, 3.7 Hz, 1H), 4.36 (d, J=15.2 Hz, 1H), 1.95-1.84 (m, 1H), 1.73-1.63 (m, 1H), 1.61-1.36 (m, 4H), 1.24 (d, J=12.2 Hz, 1H), 1.17-1.11 (m, 1H), 1.09-0.84 (m, 4H), 0.80-0.67 (m, 1H).

### 6-((1-(2-methylbenzyl)-3-oxo-1,3-dihydro-2H-pyrrolo[3,4-c]pyridin-2-yl)methyl)benzo[d]oxazol-2(3H)-one (168)

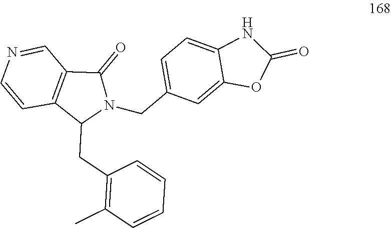 [SMILES: Cc1ccccc1CC1c2ccncc2C(=O)N1Cc1ccc2[nH]c(=O)oc2c1]

The title compound 168 was prepared according to the procedure described for compound 124 as a white solid (8 mg, 24%). MS (EST) m/z 386 [M+H]⁺. ¹H NMR (400 MHz, DMSO-d₆) δ 11.63 (s, 1H), 8.93 (s, 1H), 8.60 (d, J=5.1 Hz, 1H), 7.28-6.96 (m, 7H), 6.78 (d, J=5.1 Hz, 1H), 5.14 (d, J=15.2 Hz, 1H), 4.81-4.69 (m, l H), 4.37 (d, J=15.2 Hz, 1H), 3.46 (dd, J=13.9, 6.0 Hz, 1H), 2.79 (dd, J=13.9, 8.5 Hz, 11H), 2.10 (s, 3H).

### 2-((2-(4-hydroxybenzyl)-3-oxoisoindolin-1-yl)methyl)benzonitrile (169)

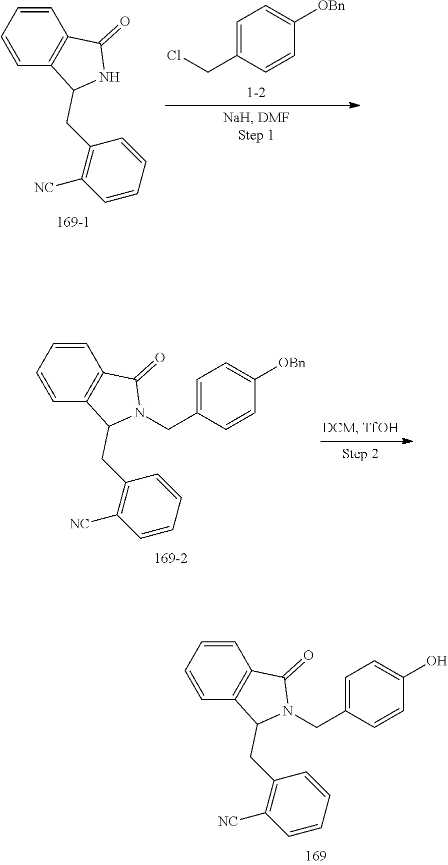 [SMILES: N#Cc1ccccc1CC1NC(=O)c2ccccc21 ; ClCc1ccc(OCc2ccccc2)cc1 ; N#Cc1ccccc1CC1c2ccccc2C(=O)N1Cc1ccc(OCc2ccccc2)cc1 ; N#Cc1ccccc1CC1c2ccccc2C(=O)N1Cc1ccc(O)cc1]

The intermediate 169-1 was prepared according to the procedure described for Int 12.

### Step 1. 2-((2-(4-(benzyloxy)benzyl)-3-oxoisoindolin-1-yl)methyl)benzonitrile (169-2)

To a solution of 2-((3-oxoisoindolin-1-yl)methyl)benzonitrile (200 mg, 0.8 mmol) in DMF (2 mL) was added NaH (21 mg, 0.88 mmol) at 0° C. under N₂. The mixture was stirred for 30 min. 1-(benzyloxy)-4-(chloromethyl)benzene (187 mg, 0.8 mmol) was added and the reaction was allowed to warm to r.t. and stirred for an additional 2 h. Water was added at 0° C. and then extracted with EtOAc. The organic layer was separated, dried over anhydrous Na₂SO₄ and concentrated under vacuum. The residue was purified by prep TLC (PE/EtOAc=1.5/1) to give 2-((2-(4-(benzyloxy)benzyl)-3-oxoisoindolin-1-yl)methyl) benzonitrile (100 mg, 27%) as a yellow solid. MS (ESI) m/z 445 [M+H]⁺.

### Step 2. 2-((2-(4-hydroxybenzyl)-3-oxoisoindolin-1-yl)methyl)benzonitrile (169)

TfOH (0.1 mL) was added to a stirred solution of 2-((2-(4-(benzyloxy)benzyl)-3-oxoisoindolin-1-yl)methyl)benzonitrile (100 mg, 0.22 mmol) in DCM (2 mL). The mixture was stirred for 14 h. The mixture was concentrated in vacuo and purified by prep HPLC to give 2-((2-(4-hydroxybenzyl)-3-oxoisoindolin-1-yl)methyl)benzonitrile (6 mg, 7.3%) as a white solid. MS (ESI) m/z 355 [M+H]⁺. ¹H NMR (400 MHz, CD₃OD) δ 7.62 (d, J=6.9 Hz, 1H), 7.53 (d, J=7.8 Hz, 1H), 7.44-7.32 (m, 3H), 7.28 (t, J=7.6 Hz, 1H), 7.08-7.03 (m, 2H), 7.00-6.93 (m, 2H), 6.67-6.59 (m, 2H), 5.12 (d, J=15.1 Hz, 1H), 4.74-4.70 (m, 1H), 4.27 (d, J=15.1 Hz, 1H), 3.51 (dd, J=14.4, 5.2 Hz, 11H), 3.19 (dd, J=14.4, 6.5 Hz, 1H).

### 3-fluoro-2-((3-oxo-2-((2-oxo-2,3-dihydrobenzo[d]oxazol-6-yl)methyl)isoindolin-1-yl) methyl)benzonitrile (170)

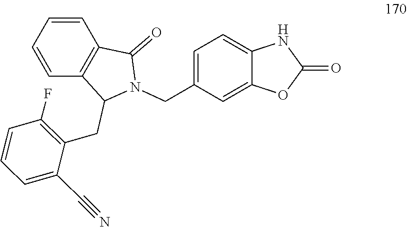 [SMILES: N#Cc1cccc(F)c1CC1c2ccccc2C(=O)N1Cc1ccc2[nH]c(=O)oc2c1]

The title compound 170 was prepared according to general procedure E as an off-white solid (63 mg, 40%). MS (ESI) m/z 414 [M+H]⁺. ¹H NMR (400 MHz, DMSO-d₆) δ 11.68 (s, 1H), 7.75-7.65 (m, 2H), 7.65-7.52 (m, 2H), 7.52-7.44 (m, 2H), 7.16 (s, 1H), 7.06-6.97 (m, 2H), 6.86-6.79 (m, 11H), 5.20 (d, J=15.2 Hz, 11H), 4.74 (dd, J=8.0, 5.6 Hz, 11H), 4.42 (d, J=15.2 Hz, 11H), 3.70 (dd, J=14.0, 5.2 Hz, 11H), 3.03 (dd, J=14.0, 8.0 Hz, 11H).

### (R)-3-fluoro-2-((3-oxo-2-((2-oxo-2,3-dihydrobenzo[d]oxazol-6-yl)methyl)isoindolin-1-yl) methyl)benzonitrile (171) and (S)-3-fluoro-2-((3-oxo-2-((2-oxo-2,3-dihydrobenzo[d]oxazol-6-yl)methyl)isoindolin-1-yl)methyl)benzonitrile (172)

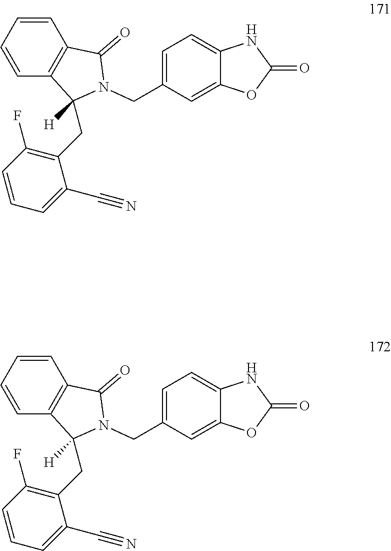 [SMILES: N#Cc1cccc(F)c1C[C@@H]1c2ccccc2C(=O)N1Cc1ccc2[nH]c(=O)oc2c1 ; N#Cc1cccc(F)c1C[C@H]1c2ccccc2C(=O)N1Cc1ccc2[nH]c(=O)oc2c1]

Chiral separation of compound 170 by chiral prep-HPLC (column: CHIRALPAK IA; column size: 2 cm×25 cm, 5 μm; mobile phase A: MeOH:DCM); mobile phase B: MTBE was conducted to afford two enantiomers: compound 171 and compound 172. 171 was a white solid, with RT=1.424 min (CHIRAL HPLC, column: CHIRALPAK IA-3; column size: 4.6*50 mm, 3 μm; mobile phase: MTBE (0.1% DEA): (MeOH:DCM=1:1)=80:20; flow: 1.0 mL/min). Mass (m/z): 414 [M+H]⁺. ¹H NMR (400 MHz, DMSO-d₆) δ 11.60 (s, 1H), 7.74-7.65 (m, 2H), 7.64-7.52 (m, 2H), 7.51-7.45 (m, 2H), 7.16 (s, 1H), 7.06-6.97 (m, 2H), 6.86-6.81 (m, 1H), 5.19 (d, J=15.2 Hz, 1H), 4.74 (dd, J=8.0, 5.6 Hz, 1H), 4.42 (d, J=15.4 Hz, 1H), 3.69 (dd, J=14.0, 5.2 Hz, 1H), 3.03 (dd, J=14.0, 8.0 Hz, 1H). 172 was a white solid, with RT=2.067 min (CHIRAL HPLC, column: CHIRALPAK IA-3; column size: 4.6*50 mm, 3 μm; mobile phase: MTBE (0.1% DEA): (MeOH:DCM=1:1)=80:20; Flow: 1.0 mL/min)Mass (m/z): 414 [M+H]⁺. ¹H NMR (400 MHz, DMSO-d₆) δ 11.59 (s, 1H), 7.74-7.64 (m, 2H), 7.64-7.50 (m, 2H), 7.53-7.43 (m, 2H), 7.15 (s, 1H), 7.06-6.97 (m, 2H), 6.87-6.79 (m, 1H), 5.19 (d, J=15.2 Hz, 1H), 4.74 (dd, J=8.0, 5.6 Hz, 11H), 4.42 (d, J=15.2 Hz, 11H), 3.69 (dd, J=14.0, 5.6 Hz, 11H), 3.03 (dd, J=14.0, 8.0 Hz, 1H). The structure of 172 was confirmed by X-ray.

### 2-(2-((3-oxo-2-((2-oxo-2,3-dihydrobenzo[d]oxazol-6-yl)methyl)isoindolin-1-yl)methyl) phenyl)acetonitrile (173)

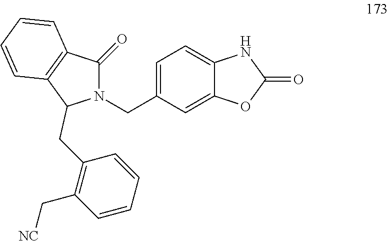 [SMILES: N#CCc1ccccc1CC1c2ccccc2C(=O)N1Cc1ccc2[nH]c(=O)oc2c1]

The title compound 173 was prepared according to general procedure E as a white solid (45 mg, 23%). MS (ESI) m/z 410 [M+H]⁺. ¹H NMR (400 MHz, DMSO-d₆) δ 11.58 (s, 1H), 7.73-7.71 (m, 11H), 7.49-7.44 (m, 2H), 7.40-7.27 (m, 3H), 7.19 (dd, J=7.6, 1.6 Hz, 1H), 7.07 (d, J=1.4 Hz, 11H), 7.03-6.97 (m, 2H), 6.84-6.82 (m, 11H), 5.12 (d, J=15.2 Hz, 1H), 4.73 (dd, J=7.9, 5.9 Hz, 1H), 4.34 (d, J=15.2 Hz, 1H), 3.83 (s, 2H), 3.46 (dd, J=14.3, 5.9 Hz, 1H), 2.88 (dd, J=14.3, 8.0 Hz, 11H).

### 2-methyl-3-((3-oxo-2-((2-oxo-2,3-dihydrobenzo[d]oxazol-6-yl)methyl)isoindolin-1-yl) methyl)benzonitrile (174)

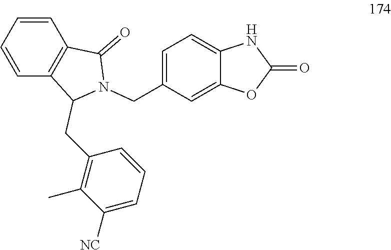 [SMILES: Cc1c(C#N)cccc1CC1c2ccccc2C(=O)N1Cc1ccc2[nH]c(=O)oc2c1]

The title compound 174 was prepared according to general procedure E using 3-(bromomethyl)-2-methylbenzonitrile (preparation according to the procedure described for 164-3) as a starting material to afford a white solid (136 mg, 52%). MS (ESI) m/z 410 [M+H]⁺. ¹H NMR (400 MHz, DMSO-d₆) δ 11.60 (s, 1H), 7.75-7.65 (m, 2H), 7.51-7.45 (m, 2H), 7.41 (d, J=7.8 Hz, 1H), 7.31 (t, J=7.7 Hz, 1H), 7.05 (d, J=1.5 Hz, 11H), 7.01 (d, J=7.9 Hz, 1H), 6.96 (d, J=8.0 Hz, 1H), 6.90-6.85 (m, 1H), 5.09 (d, J=15.3 Hz, 1H), 4.74 (dd, J=7.8, 5.8 Hz, 1H), 4.39 (d, J=15.3 Hz, 1H), 3.49 (dd, J=14.2, 5.8 Hz, 1H), 2.94 (dd, J=14.2, 7.8 Hz, 1H), 2.26 (s, 3H).

### 6-((1-((4-chloropyridin-3-yl)methyl)-3-oxoisoindolin-2-yl)methyl)benzo[d]oxazol-2(3H)-one (175)

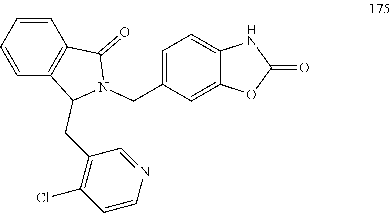 [SMILES: O=C1c2ccccc2C(Cc2cnccc2Cl)N1Cc1ccc2[nH]c(=O)oc2c1]

The title compound 175 was prepared according to general procedure E as a white solid (36 mg, 23.7%). MS (ESI) m/z 406 [M+H]⁺. ¹H NMR (400 MHz, DMSO-d₆) δ 11.61 (s, 1H), 8.38 (d, J=5.3 Hz, 1H), 8.29 (s, 1H), 7.68 (d, J=6.7 Hz, 1H), 7.57-7.45 (m, 2H), 7.47 (d, J=2.6 Hz, 1H), 7.13 (s, 1H), 7.08-7.02 (m, 1H), 7.06-6.97 (m, 2H), 5.17 (d, J=15.2 Hz, 1H), 4.82 (dd, J=7.1, 5.1 Hz, 1H), 4.47 (d, J=15.2 Hz, 1H), 3.59 (dd, J=14.2, 5.1 Hz, 1H), 3.13 (dd, J=14.2, 7.2 Hz, 1H).

### 6-((1-((3-methylpyridin-2-yl)methyl)-3-oxoisoindolin-2-yl)methyl)benzo[d]oxazol-2(3H)-one (176)

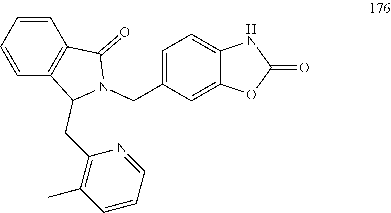 [SMILES: Cc1cccnc1CC1c2ccccc2C(=O)N1Cc1ccc2[nH]c(=O)oc2c1]

The title compound 176 was prepared according to general procedure E as a white solid (71 mg, 35.8%). MS (ESI) m/z 386 [M+H]⁺. ¹H NMR (400 MHz, DMSO-d₆) δ 11.64 (s, 1H), 8.39 (d, J=4.9 Hz, 1H), 7.76-7.71 (m, 1H), 7.54 (d, J=7.8 Hz, 1H), 7.52-7.42 (m, 2H), 7.22 (dd, J=7.6, 4.8 Hz, 1H), 7.02-6.94 (m, 3H), 6.91 (d, J=8.0 Hz, 1H), 5.19 (dd, J=7.6, 5.8 Hz, 1H), 5.04 (d, J=15.3 Hz, 1H), 4.39 (d, J=15.3 Hz, 1H), 3.44 (dd, J=15.1, 5.8 Hz, 1H), 2.97 (dd, J=15.1, 7.7 Hz, 1H), 2.05 (s, 3H).

176-2 was prepared according to the procedure described for step 2 of 174-3 preparation.

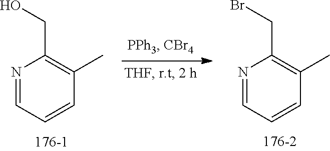

### (R)-6-((1-((3-methylpyridin-2-yl)methyl)-3-oxoisoindolin-2-yl)methyl)benzo[d]oxazol-2(3H)-one (177) and (S)-6-((1-((3-methylpyridin-2-yl)methyl)-3-oxoisoindolin-2-yl)methyl)benzo[d]oxazol-2(3H)-one (178)

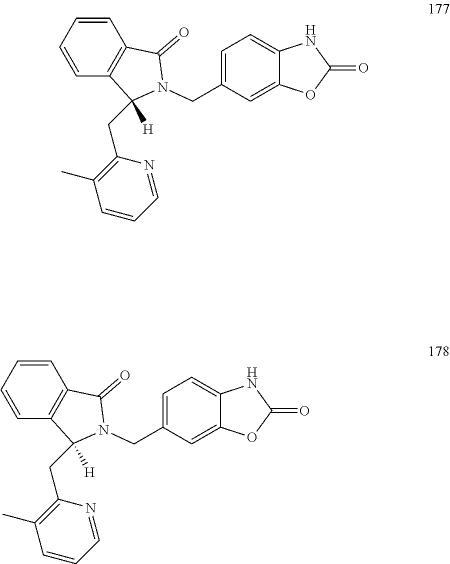 [SMILES: Cc1cccnc1C[C@@H]1c2ccccc2C(=O)N1Cc1ccc2[nH]c(=O)oc2c1 ; Cc1cccnc1C[C@H]1c2ccccc2C(=O)N1Cc1ccc2[nH]c(=O)oc2c1]

Chiral separation of compound 176 by chiral prep-HPLC (Column: CHIRALPAK IF; Column size: 2 cm×25 cm, 5 um; Mobile phase A: MeOH:DCM; Mobile phase B: MtBE; Flow rate: 20 mL/min; Detector: 220 nm.) to afford two enantiomers: compound 177 and 178. 177 was a white solid, with RT=1.571 min (CHIRAL HPLC, column: CHIRALPAK IF-3; column size: 4.6*50 mm, 3 μm; mobile phase: MTBE (0.1% DEA):(MeOH:DCM=1:1)=80:20; flow: 1.0 mL/min)). MS (ESI) m/z 386 [M+H]⁺. ¹H NMR (400 MHz, DMSO-d₆) δ 11.57 (s, 1H), 8.38 (d, J=4.8 Hz, 1H), 7.77-7.70 (m, 1H), 7.54 (d, J=7.6 Hz, 1H), 7.51-7.43 (m, 2H), 7.21 (dd, J=7.6, 4.8 Hz, 1H), 6.99 (m, 3H), 6.91 (d, J=8.0 Hz, 1H), 5.19 (t, J=6.7 Hz, 1H), 5.04 (d, J=15.3 Hz, 1H), 4.39 (d, J=15.3 Hz, 1H), 3.43 (dd, J=15.1, 5.8 Hz, 1H), 2.98 (dd, J=15.1, 7.6 Hz, 1H), 2.05 (s, 3H). 178 was a white solid, with RT=2.158 min (CHIRAL HPLC, column: CHIRALPAK IF-3; column size: 4.6*50 mm, 3 μm; mobile phase: MTBE (0.1% DEA): (MeOH:DCM=1:1)=80:20; flow: 1.0 mL/min). MS (ESI) m/z 386 [M+H]⁺. ¹H NMR (400 MHz, DMSO-d₆) δ 11.57 (s, 1H), 8.38 (d, J=4.8 Hz, 1H), 7.77-7.70 (m, 1H), 7.54 (d, J=7.6 Hz, 1H), 7.51-7.43 (m, 2H), 7.21 (dd, J=7.6, 4.8 Hz, 1H), 6.99 (m, 3H), 6.91 (d, J=8.0 Hz, 1H), 5.19 (dd, J=7.6, 5.6 Hz, 1H), 5.04 (d, J=15.3 Hz, 1H), 4.39 (d, J=15.3 Hz, 1H), 3.43 (dd, J=15.1, 5.8 Hz, 1H), 2.98 (dd, J=15.1, 7.6 Hz, 1H), 2.05 (s, 3H). The structure of 178 was confirmed by X-ray.

### 6-((1-((2-methylpyridin-4-yl)methyl)-3-oxoisoindolin-2-yl)methyl)benzo[d]oxazol-2(3H)-one (179)

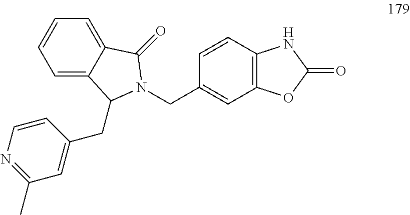 [SMILES: Cc1cc(CC2c3ccccc3C(=O)N2Cc2ccc3[nH]c(=O)oc3c2)ccn1]

The title compound 179 was prepared according to general procedure E as a white solid (25 mg, 26%). MS (ESI) m/z 386 [M+H]⁺. ¹H NMR (400 MHz, DMSO-d₆) b 11.63 (s, 1H), 8.18 (d, J=5.1 Hz, 1H), 7.65-7.54 (m, 2H), 7. 7.48-7.42 (m, 2H), 7.20 (s, 1H), 7.05 (d, J=1.8 Hz, 2H), 6.81 (s, 1H), 6.74 (d, J=5.2 Hz, 1H), 5.09 (d, J=15.2 Hz, 1H), 4.81 (t, J=5.1 Hz, 1H), 4.47 (d, J=15.2 Hz, 1H), 3.36-3.29 (m, 1H), 3.24 (dd, J=14.1, 5.9 Hz, 1H), 2.29 (s, 3H).

### 6-((1-(3-methoxybenzyl)-3-oxoisoindolin-2-yl)methyl)benzo[d]oxazol-2(3H)-one (180)

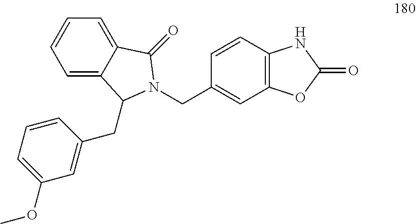 [SMILES: COc1cccc(CC2c3ccccc3C(=O)N2Cc2ccc3[nH]c(=O)oc3c2)c1]

The title compound 180 was prepared according to general procedure E as a white solid (47 mg, 40.2%). MS (ESI) m/z 401 [M+H]⁺. ¹H NMR (400 MHz, DMSO-d₆) δ 11.63 (s, 1H), 7.60 (d, J=7.5 Hz, 1H), 7.55 (td, J=7.5, 1.3 Hz, 1H), 7.44 (t, J=7.4 Hz, 1H), 7.37 (d, J=7.6 Hz, 1H), 7.17 (s, 1H), 7.10-7.00 (m, 3H), 6.70 (dd, J=8.2, 2.6 Hz, 1H), 6.60-6.53 (m, 1H), 6.48 (t, J=2.0 Hz, 1H), 5.11 (d, J=15.1 Hz, 1H), 4.73 (dd, J=6.1, 4.3 Hz, 1H), 4.42 (d, J=15.1 Hz, 1H), 3.58 (s, 3H), 3.31-3.27 (m, 1H), 3.16 (dd, J=14.1, 6.2 Hz, 1H).

### 6-((1-((3-isopropylpyridin-2-yl)methyl)-3-oxoisoindolin-2-yl)methyl)benzo[d]oxazol-2(3H)-one (181)

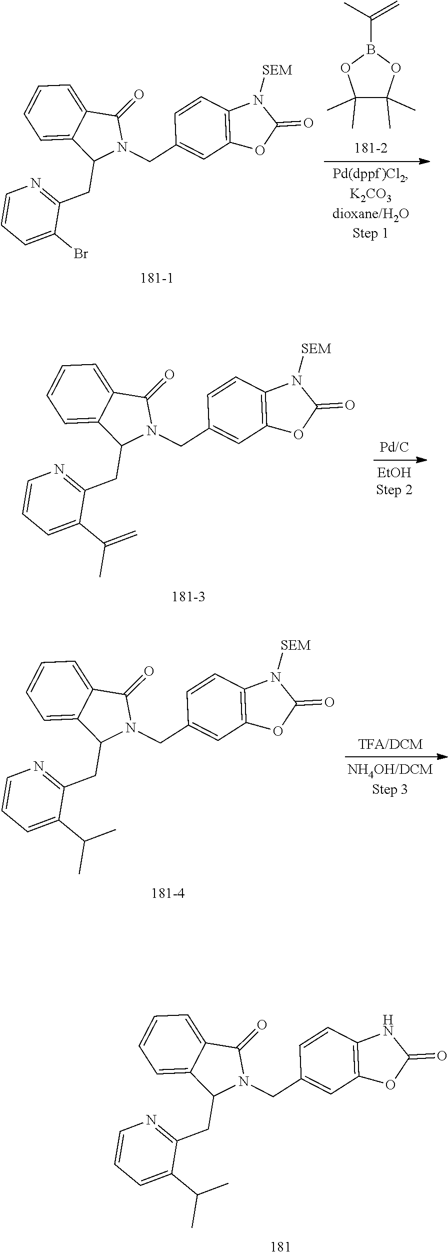

181-1 was prepared according to the procedure described for step 1 in general procedure E.

### Step 1. 6-((1-oxo-3-((3-(prop-1-en-2-yl)pyridin-2-yl)methyl)isoindolin-2-yl)methyl)-3-((2-(trimethylsilyl)ethoxy)methyl)benzo[d]oxazol-2(3H)-one (181-3)

To a solution of 6-((1-((3-bromopyridin-2-yl)methyl)-3-oxoisoindolin-2-yl)methyl)-3-((2-(trimethylsilyl)ethoxy)methyl)benzo[d]oxazol-2(3H)-one (100 mg, 0.17 mmol) in dioxane (3 ml) and water (1 ml) were added Pd(dppf)Cl₂ (13 mg, 0.02 mmol) and K₂CO₃ (48 mg, 0.35 mmol) at r.t. under N₂. The resulting mixture was stirred for 3 h at 100° C. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=1/3) to afford the title compound (60 mg, 64.1%) as a yellow oil. MS (ESI) m/z 542 [M+H]⁺.

### Step 2. 6-((1-((3-isopropylpyridin-2-yl)methyl)-3-oxoisoindolin-2-yl)methyl)-3-((2-(trimethylsilyl)ethoxy)methyl)benzo[d]oxazol-2(3H)-one (181-4)

To a solution of 6-((1-oxo-3-((3-(prop-1-en-2-yl)pyridin-2-yl)methyl)isoindolin-2-yl) methyl)-3-((2-(trimethylsilyl)ethoxy)methyl)benzo[d]oxazol-2(3H)-one (60 mg, 0.11 mmol) in EtOH (5 mL) was added Pd/C (10 mg). The mixture was evacuated and backfilled three times with hydrogen. The resulting mixture was stirred at r.t. overnight. The reaction mixture was filtered off and the filtrate was concentrated to afford the title compound (50 mg, 83%) as a yellow oil. MS (ESI) m/z 544 [M+H]⁺.

### Step 3. 6-((1-((3-isopropylpyridin-2-yl)methyl)-3-oxoisoindolin-2-yl)methyl)benzo[d]oxazol-2(3H)-one (181)

To a solution of 6-((1-((3-isopropylpyridin-2-yl)methyl)-3-oxoisoindolin-2-yl)methyl)-3-((2-(trimethylsilyl)ethoxy)methyl)benzo[d]oxazol-2(3H)-one (50 mg, 0.09 mmol) in DCM (5 mL) was added trifluoroacetic acid (2 mL). The mixture was stirred at r.t. for 2h, and then concentrated under vacuum. The residue was dissolved with DCM (2 mL), and NH₄₀H (1 mL) was added dropwise. The mixture was stirred at r.t. for 1 h. The reaction mixture was concentrated, and the residue was purified by Prep-HPLC to afford 6-((1-((3-methylpyridin-2-yl) methyl)-3-oxoisoindolin-2-yl) methyl) benzo[d]oxazol-2(3H)-one (17 mg, 44.7%) as a white solid. MS (ESI) m/z 414 [M+H]⁺. ¹H NMR (400 MHz, DMSO-do) δ 11.59 (s, 1H), 8.40 (dd, J=4.7, 1.7 Hz, 1H), 7.77-7.69 (m, 1H), 7.67 (dd, J=7.9, 1.7 Hz, 1H), 7.51-7.39 (m, 2H), 7.30 (dd, J=7.9, 4.7 Hz, 1H), 7.09-6.97 (m, 2H), 6.92 (dd, J=7.9, 1.6 Hz, 1H), 6.80 (d, J=7.1 Hz, 1H), 5.13 (dd, J=7.8, 5.8 Hz, 1H), 5.08 (d, J=15.3 Hz, 1H), 4.39 (d, J=15.3 Hz, 1H), 3.52 (dd, J=14.7, 5.9 Hz, 1H), 3.00 (dd, J=14.7, 8.0 Hz, 11H), 2.84-2.71 (m, 11H), 0.98 (d, J=6.8 Hz, 3H), 0.95 (d, J=6.8 Hz, 3H).

### 6-((1-((3-ethylpyridin-2-yl)methyl)-3-oxoisoindolin-2-yl)methyl)benzo[d]oxazol-2(3H)-one (182)

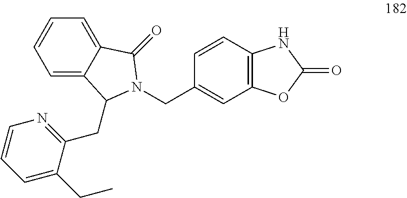 [SMILES: CCc1cccnc1CC1c2ccccc2C(=O)N1Cc1ccc2[nH]c(=O)oc2c1]

The title compound 182 was prepared according to the procedure described for compound 181 as a white solid (7 mg, 7.7%). MS (ESI) m/z 400 [M+H]⁺. ¹H NMR (400 MHz, DMSO-d₆) δ 11.58 (s, 1H), 8.41 (dd, J=4.8, 1.8 Hz, 1H), 7.74 (dd, J=6.7, 1.8 Hz, 1H), 7.56 (dd, J=7.7, 1.7 Hz, 1H), 7.53-7.40 (m, 2H), 7.26 (dd, J=7.7, 4.7 Hz, 1H), 7.03-6.96 (m, 2H), 6.95-6.84 (m, 2H), 5.22 (dd, J=7.6, 6.0 Hz, 1H), 5.04 (d, J=15.3 Hz, 11H), 4.38 (d, J=15.3 Hz, 1H), 3.45 (dd, J=15.0, 6.0 Hz, 1H), 3.28 (s, 1H), 2.97 (dd, J=15.0, 7.8 Hz, 1H), 2.46-2.33 (m, 1H), 0.93 (t, J=7.5 Hz, 3H).

### 6-((1-((2-fluoropyridin-3-yl)methyl)-3-oxoisoindolin-2-yl)methyl)benzo[d]oxazol-2(3H)-one (183)

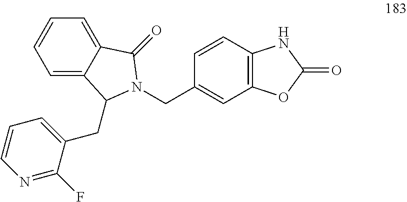 [SMILES: O=C1c2ccccc2C(Cc2cccnc2F)N1Cc1ccc2[nH]c(=O)oc2c1]

The title compound 183 was prepared according to general procedure E as a white solid (27 mg, 32%). MS (ESI) m/z 390 [M+H]⁺. ¹H NMR (400 MHz, DMSO-d₆) δ 11.63 (s, 1H), 8.00 (dd, J=4.9, 1.8 Hz, 1H), 7.62-7.47 (m, 3H), 7.43 (t, J=7.4 Hz, 1H), 7.37 (d, J=7.6 Hz, 1H), 7.20 (s, 1H), 7.16-7.11 (m, 1H), 7.10-7.00 (m, 2H), 5.18 (d, J=15.2 Hz, 1H), 4.78 (t, J=5.0 Hz, 1H), 4.48 (d, J=15.2 Hz, 1H), 3.44 (dd, J=14.4, 4.2 Hz, 1H), 3.28 (dd, J=14.4, 5.6 Hz, 1H).

### 6-((1-((3-cyclopropylpyridin-2-yl)methyl)-3-oxoisoindolin-2-yl)methyl)benzo[d]oxazol-2(3H)-one (184)

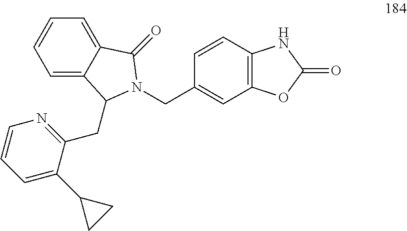 [SMILES: O=C1c2ccccc2C(Cc2ncccc2C2CC2)N1Cc1ccc2[nH]c(=O)oc2c1]

The title compound 184 was prepared according to general procedure E as a white solid (8 mg, 7.9%). MS (ESI) m/z 412 [M+H]⁺. ¹H NMR (400 MHz, DMSO-d₆) δ 11.58 (s, 1H), 8.38 (dd, J=4.9, 1.6 Hz, 1H), 7.74 (dd, J=6.4, 2.0 Hz, 1H), 7.51-7.42 (m, 2H), 7.37 (d, J=7.9 Hz, 1H), 7.25 (t, J=6.3 Hz, 1H), 7.02-6.96 (m, 2H), 6.94-6.87 (m, 2H), 5.21 (t, J=6.8 Hz, 1H), 5.08 (d, J=15.3 Hz, 1H), 4.39 (d, J=15.3 Hz, 1H), 3.63 (dd, J=14.9, 6.0 Hz, 1H), 3.18 (dd, J=14.9, 7.8 Hz, 1H), 1.76-1.66 (m, 1H), 0.78-0.68 (m, 2H), 0.54-0.38 (m, 2H).

The intermediate 184-3 was prepared as follows:

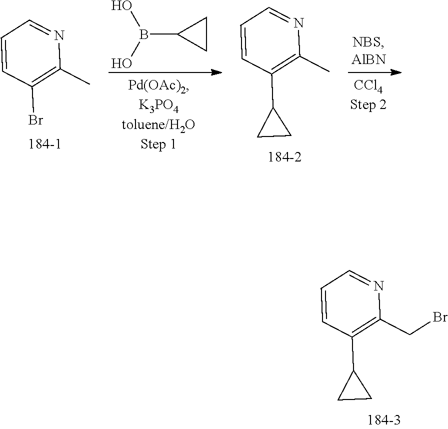

Step 1 was performed according to the procedure outlined for step 1 for the preparation of compound 181.

Step 2 was performed according to the procedure outlined for step 3 for the preparation of Int 1.

### rel-(S)-6-((5-((3-methoxyazetidin-1-yl)methyl)-3-methyl-1-oxoisoindolin-2-yl)methyl) benzo[d]oxazol-2(3H)-one (185)

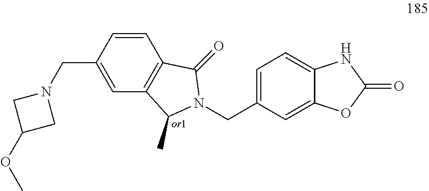 [SMILES: COC1CN(Cc2ccc3c(c2)[C@H](C)N(Cc2ccc4[nH]c(=O)oc4c2)C3=O)C1]

The title compound 185 was prepared according to compound 96 using the stereoisomer of Int 10 or Int 11 that has RT of 1.969 min as a starting material to afford a white solid (2 mg, 9%). MS (ESI) m/z 394 [M+H]⁺. ¹H NMR (400 MHz, DMSO-d₆) δ 11.60 (s, 1H), 7.64 (d, J=7.7 Hz, 1H), 7.47 (s, 1H), 7.39 (d, J=7.9 Hz, 1H), 7.23 (s, 1H), 7.10-7.02 (m, 2H), 5.00 (d, J=15.2 Hz, 1H), 4.43-4.36 (m, 2H), 4.01-3.91 (m, 1H), 3.70 (s, 2H), 3.51 (s, 2H), 3.14 (s, 3H), 2.90 (s, 2H), 1.38 (d, J=6.7 Hz, 3H).

### rel-(S)-3-methyl-1-oxo-2-((2-oxo-2,3-dihydrobenzo[d]oxazol-6-yl)methyl)isoindoline-5-carbonitrile (186)

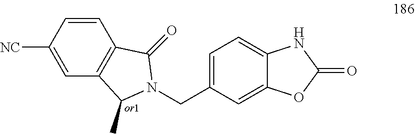 [SMILES: C[C@H]1c2cc(C#N)ccc2C(=O)N1Cc1ccc2[nH]c(=O)oc2c1]

The title compound 186 was prepared according to the procedure described for compound 163 using the stereoisomer of Int 10 or Int 11 that has RT of 1.969 min as a starting material to afford a white solid (4 mg, 9.4%). MS (ESI) m/z 320 [M+H]⁺. ¹H NMR (400 MHz, CD₃OD) δ 7.90-7.84 (m, 2H), 7.79 (dd, J=7.9, 1.3 Hz, 1H), 7.14 (d, J=1.6 Hz, 1H), 7.08 (dd, J=8.0, 1.6 Hz, 1H), 6.96 (d, J=8.0 Hz, 1H), 5.07 (d, J=15.2 Hz, 1H), 4.51 (q, J=6.8 Hz, 1H), 4.43 (d, J=15.2 Hz, 1H), 1.40 (d, J=6.8 Hz, 3H).

### rel-(S)-6-((5-((2-oxa-6-azaspiro[3.3]heptan-6-yl)methyl)-3-methyl-1-oxoisoindolin-2-yl) methyl)benzo[d]oxazol-2(3H)-one (187)

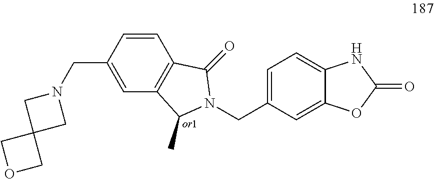 [SMILES: C[C@H]1c2cc(CN3CC4(COC4)C3)ccc2C(=O)N1Cc1ccc2[nH]c(=O)oc2c1]

The title compound 187 was prepared according to the procedure described for compound 96 using the stereoisomer of Int 10 or Int 11 that has RT of 1.969 min as a starting material to afford a white solid (4 mg, 9%). MS (ESI) m/z 406 [M+H]⁺. 1H NMR (400 MHz, CD₃OD) δ 7.73 (d, J=7.7 Hz, 11H), 7.46-7.40 (m, 2H), 7.10 (s, 1H), 7.07 (d, J=8.1 Hz, 1H), 6.95 (d, J=8.0 Hz, 1H), 5.06 (d, J=15.2 Hz, 1H), 4.66 (s, 4H), 4.45-4.37 (m, 2H), 4.01 (s, 2H), 3.86 (s, 4H), 1.38 (d, J=6.7 Hz, 3H).

### rel-6-(((3S)-5-((3-methoxypyrrolidin-1-yl)methyl)-3-methyl-1-oxoisoindolin-2-yl)methyl) benzo[d]oxazol-2(3H)-one (188)

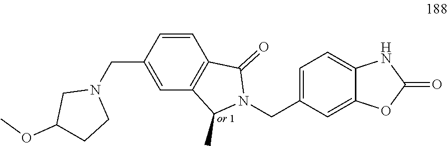 [SMILES: COC1CCN(Cc2ccc3c(c2)[C@H](C)N(Cc2ccc4[nH]c(=O)oc4c2)C3=O)C1]

The title compound 188 was prepared according to compound 96 using the stereoisomer of Int 10 or Int 11 that has RT of 1.969 min as a starting material to afford a white solid (7 mg, 17%). MS (ESI) m/z 408 [M+H]⁺. ¹H NMR (400 MHz, CD₃OD) δ 7.79 (d, J=7.8 Hz, 1H), 7.60-7.54 (m, 2H), 7.12-7.06 (m, 2H), 6.95 (d, J=8.0 Hz, 1H), 5.07 (d, J=15.2 Hz, 1H), 4.41 (dd, J=13.6, 6.8 Hz, 1H), 4.41 (d, J=15.2 Hz, 1H), 4.34-4.30 (m, 2H), 4.07-4.03 (m, 1H), 3.41-3.23 (m, 4H), 3.21 (s, 3H), 2.19-2.05 (m, 2H), 1.40 (d, J=6.7 Hz, 3H).

### 2-((2-((1H-benzo[d][1,2,3]triazol-5-yl)methyl)-3-oxoisoindolin-1-yl)methyl)benzonitrile (189)

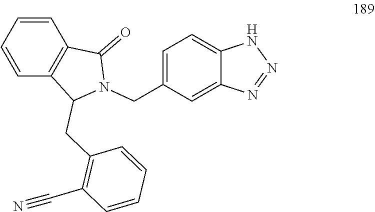 [SMILES: N#Cc1ccccc1CC1c2ccccc2C(=O)N1Cc1ccc2[nH]nnc2c1]

The title compound 189 was prepared according to general procedure C using 169-1 as a starting material to afford an off-white solid (10 mg, 13%). MS (ESI) m/z 380 [M+H]⁺. ¹H NMR (400 MHz, DMSO-d₆) δ 15.56 (s, 1H), 7.86 (d, J=8.5 Hz, 1H), 7.73-7.64 (m, 3H), 7.61-7.44 (m, 3H), 7.41 (t, J=7.6 Hz, 1H), 7.33-7.24 (m, 2H), 7.13 (d, J=7.4 Hz, 1H), 5.32 (d, J=15.4 Hz, 1H), 4.89 (t, J=6.0 Hz, 1H), 4.65 (d, J=15.4 Hz, 1H), 3.61 (dd, J=14.4, 5.2 Hz, 1H), 3.32 (dd, J=14.4, 7.2 Hz, 1H).

### 3-chloro-2-((3-oxo-2-((2-oxo-2,3-dihydrobenzo[d]oxazol-6-yl)methyl)isoindolin-1-yl) methyl)benzonitrile (190)

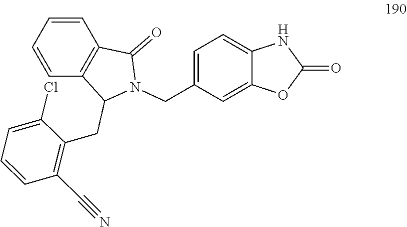 [SMILES: N#Cc1cccc(Cl)c1CC1c2ccccc2C(=O)N1Cc1ccc2[nH]c(=O)oc2c1]

The title compound 190 was prepared according to general procedure E as a light yellow solid (78 mg, 49%). MS (ESI) m/z 430 [M+H]⁺. ¹H NMR (400 MHz, DMSO-d₆) δ 11.66 (s, 1H), 7.92-7.85 (m, 2H), 7.79 (d, J=7.4 Hz, 1H), 7.59 (t, J=8.0 Hz, 1H), 7.52 (t, J=7.4 Hz, 1H), 7.45 (t, J=7.5 Hz, 1H), 7.11 (d, J=1.5 Hz, 1H), 7.06-6.95 (m, 2H), 6.51 (d, J=7.5 Hz, 1H), 5.20 (d, J=15.2 Hz, 1H), 4.79 (dd, J=9.2, 6.0 Hz, 1H), 4.36 (d, J=15.3 Hz, 1H), 3.79 (dd, J=13.6, 6.0 Hz, 1H), 2.91 (dd, J=13.6, 9.2 Hz, 1H).

### 6-((1-((3-(difluoromethyl)pyridin-2-yl)methyl)-3-oxoisoindolin-2-yl)methyl)benzo[d]oxazol-2(3H)-one (191)

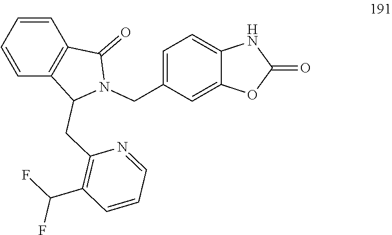 [SMILES: O=C1c2ccccc2C(Cc2ncccc2C(F)F)N1Cc1ccc2[nH]c(=O)oc2c1]

The title compound 191 was prepared according to general procedure E as an off-white solid (39 mg, 24%). MS (ESI) m/z 422 [M+H]⁺. ¹H NMR (400 MHz, DMSO-d₆) δ 11.61 (s, 1H), 8.70 (d, 15 J=4.8 Hz, 1H), 7.93 (d, J=7.8 Hz, 1H), 7.78-7.69 (m, 1H), 7.53-7.43 (m, 3H), 7.06 (t, J=52 Hz, 1H), 7.03-6.94 (m, 3H), 6.90-6.83 (m, 1H), 5.28 (t, J=6.6 Hz, 1H), 4.99 (d, J=15.5 Hz, 1H), 4.36 (d, J=15.5 Hz, 1H), 3.56 (dd, J=15.4, 6.2 Hz, 1H), 3.21 (dd, J=15.4, 7.0 Hz, 1H).

The intermediate 191-3 was prepared as follows:

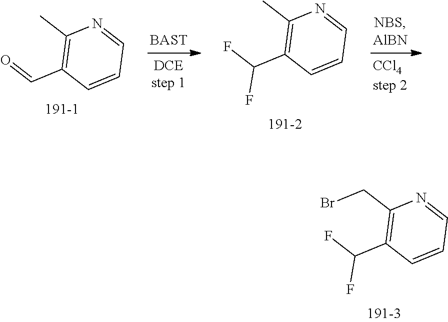

### Step 1. 3-(difluoromethyl)-2-methylpyridine (191-2)

To a solution of 2-methylnicotinaldehyde (1.36 g, 11.2 mmol) in DCE (20 mL) was added BAST (7.45 g, 33.7 mmol) under N₂. The mixture was stirred at 85° C. for 14 h. The mixture was concentrated under vacuum. The residue was taken in EtOAc, then adjusted to pH 7 with NaHCO₃ (aq.) and extracted with EtOAc. The organic layer was separated, dried over anhydrous Na₂SO₄ and concentrated. The residue was purified by silica gel column chromatography (PE/EA=9/1) to give 3-(difluoromethyl)-2-methylpyridine (800 mg, 49%) as a yellow oil. MS (ESI) m/z 144 [M+H]⁺.

### Step 2. 2-(bromomethyl)-3-(difluoromethyl)pyridine (191-3)

To a solution of 3-(difluoromethyl)-2-methylpyridine (800 mg, 5.59 mmol) in CCl₄ (10 mL) were added NBS (1.09 g, 6.15 mmol) and AIBN (92 mg, 0.55 mmol) at r.t. The mixture was stirred for 14 h at 80° C. After the reaction was completed, the solid was filtered, and the filtrate was concentrated. The residue was purified by silica gel flash column chromatography (PE/EtOAc=1/10) to afford 2-(bromomethyl)-3-(difluoromethyl)pyridine (129 mg, 10%) as a yellow oil. MS (ESI) m/z 222 [M+H]⁺.

### (R)-6-((1-((3-(difluoromethyl)pyridin-2-yl)methyl)-3-oxoisoindolin-2-yl)methyl)benzo[d]oxazol-2(3H)-one (192) and (S)-6-((1-((3-(difluoromethyl)pyridin-2-yl)methyl)-3-oxoisoindolin-2-yl)methyl)benzo[d]oxazol-2(3H)-one (193)

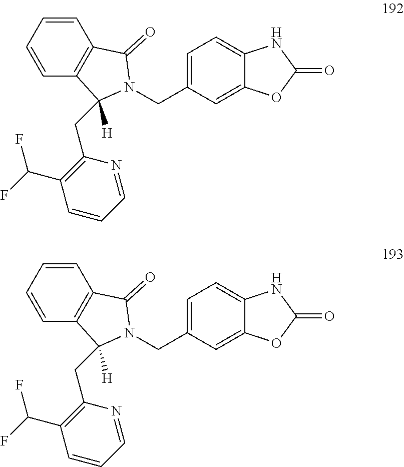 [SMILES: O=C1c2ccccc2[C@@H](Cc2ncccc2C(F)F)N1Cc1ccc2[nH]c(=O)oc2c1 ; O=C1c2ccccc2[C@H](Cc2ncccc2C(F)F)N1Cc1ccc2[nH]c(=O)oc2c1]

Chiral separation of 191 by chiral prep-HPLC (column: CHIRALPAK IA; column size: 2 cm×25 cm, 5 μm; mobile phase A: MTBE; mobile phase B: MeOH:DCM) was conducted to afford two enantiomers: compound 192 and compound 193. One of the two enantiomers was a white solid, with RT=1.350 min (CHIRAL HPLC, column: CHIRALPAK IA-3; column size: 4.6*50 mm, 3 μm; mobile phase: MTBE (0.1% DEA): (MeOH:DCM=1:1)=80:20; flow: 1.0 mL/min). MS (ESI) m/z: 422 [M+H]⁺. ¹H NMR (400 MHz, CD₃OD) δ 8.56 (d, J=4.9 Hz, 1H), 7.83 (d, J=7.8 Hz, 1H), 7.78-7.70 (m, 1H), 7.45-7.31 (m, 3H), 6.94-6.76 (m, 4H), 6.65 (t, J=56 Hz, 1H), 5.27 (t, J=6.6 Hz, 1H), 5.01 (d, J=15.4 Hz, 1H), 4.36 (d, J=15.5 Hz, 1H), 3.44 (dd, J=15.3, 6.5 Hz, 1H), 3.14 (dd, J=15.3, 6.7 Hz, 1H). The other one of the two enantiomers was a white solid, with RT=1.680 min (CHIRAL HPLC, column: CHIRALPAK IA-3; column size: 4.6*50 mm, 3 μm; mobile phase: MTBE (0.1% DEA): (MeOH:DCM=1:1)=80:20; Flow: 1.0 mL/min). MS (ESI) m/z: 422 [M+H]⁺. ¹H NMR (400 MHz, CD₃OD) δ 8.56 (d, J=4.9 Hz, 1H), 7.83 (d, J=7.8 Hz, 1H), 7.78-7.70 (m, 1H), 7.45-7.31 (m, 3H), 6.94-6.76 (m, 4H), 6.65 (t, J=56 Hz, 1H), 5.27 (t, J=6.6 Hz, 1H), 5.01 (d, J=15.4 Hz, 1H), 4.36 (d, J=15.5 Hz, 1H), 3.44 (dd, J=15.3, 6.5 Hz, 1H), 3.14 (dd, J=15.3, 6.7 Hz, 1H). RT=1.680 min.

### 6-((1-((5-fluoropyridin-2-yl)methyl)-3-oxoisoindolin-2-yl)methyl)benzo[d]oxazol-2(3H)-one (194)

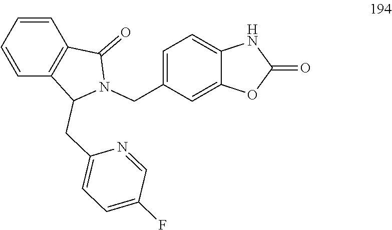 [SMILES: O=C1c2ccccc2C(Cc2ccc(F)cn2)N1Cc1ccc2[nH]c(=O)oc2c1]

The title compound 194 was prepared according to general procedure E as a light yellow solid (74 mg, 47%). MS (ESI) m/z 390 [M+H]⁺. ¹H NMR (400 MHz, DMSO-d₆) δ 11.64 (s, 1H), 8.44 (d, J=2.9 Hz, 1H), 7.64 (d, J=7.4 Hz, 1H), 7.58-7.48 (m, 2H), 7.45 (t, J=7.4 Hz, 1H), 7.19 (d, J=7.5 Hz, 1H), 7.14 (s, 1H), 7.11-6.96 (m, 3H), 5.11 (d, J=15.3 Hz, 1H), 4.91 (dd, J=6.6, 4.8 Hz, 1H), 4.45 (d, J=15.4 Hz, 1H), 3.52 (dd, J=14.2, 4.8 Hz, 1H), 3.24 (dd, J=14.2, 6.7 Hz, 1H).

### 2-((2-(3-fluoro-4-hydroxybenzyl)-3-oxoisoindolin-1-yl)methyl)benzonitrile (195)

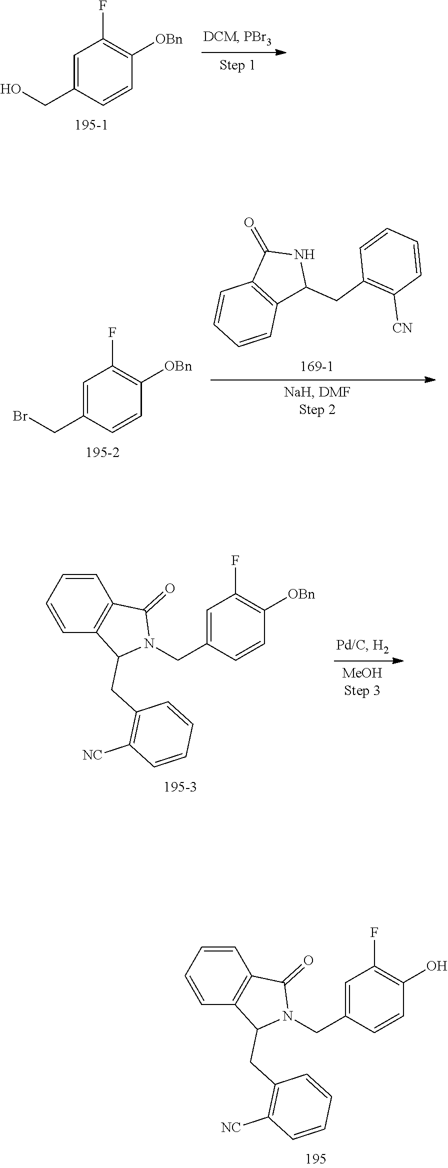 [SMILES: OCc1ccc(OCc2ccccc2)c(F)c1 ; Fc1cc(CBr)ccc1OCc1ccccc1 ; N#Cc1ccccc1CC1NC(=O)c2ccccc21 ; N#Cc1ccccc1CC1c2ccccc2C(=O)N1Cc1ccc(OCc2ccccc2)c(F)c1 ; N#Cc1ccccc1CC1c2ccccc2C(=O)N1Cc1ccc(O)c(F)c1]

### Step 1. 1-(benzyloxy)-4-(bromomethyl)-2-fluorobenzene (195-2)

To a mixture of (4-(benzyloxy)-3-fluorophenyl)methanol (100 mg, 0.43 mmol) in DCM (3 mL) was added PBr₃ (70 mg, 0.25 mmol) dropwise at 0 T. The resulting mixture was allowed to warm to r.t. and stirred for 1.5 h. The reaction mixture was adjusted to pH 7 using NaHCO₃ (aq.). The aqueous layer was separated and the organic layer was washed with brine, dried over anhydrous Na₂SO₄ and concentrated in vacuo to give 1-benzyloxy-4-(bromomethyl)-2-fluoro-benzene (80 mg) as a white solid, which was used in the next step without any further purification. MS (ESI) m/z 295 [M+H]⁺.

Step 2 was performed according to the procedure outlined for step 1 in general procedure A.

Step 3 was performed according to the procedure outlined for step 2 in general procedure A to 25 obtain compound 195 as a white solid (12 mg, 20%). MS (ESI) m/z 373 [M+H]⁺. ¹H NMR (400 MHz, DMSO-d₆) δ 9.82 (s, 1H), 7.73 (d, J=7.7 Hz, 1H), 7.64 (d, J=7.4 Hz, 1H), 7.60-7.48 (m, 2H), 7.47-7.37 (m, 2H), 7.27 (d, J=7.8 Hz, 1H), 7.14 (d, J=7.5 Hz, 1H), 6.96 (dd, J=12.0, 1.9 Hz, 1H), 6.91-6.81 (m, 2H), 5.05 (d, J=15.2 Hz, 1H), 4.85 (t, J=6.0 Hz, 1H), 4.34 (d, J=15.2 Hz, 1H), 3.58 (dd, J=14.4, 5.2 Hz, 1H), 3.27 (dd, J=14.4, 6.4 Hz, 1H).

### 6-((1-((5-chloropyridin-2-yl)methyl)-3-oxoisoindolin-2-yl)methyl)benzo[d]oxazol-2(3H)-one (196)

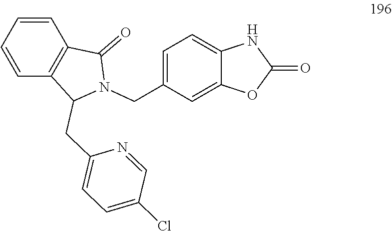 [SMILES: O=C1c2ccccc2C(Cc2ccc(Cl)cn2)N1Cc1ccc2[nH]c(=O)oc2c1]

The title compound 196 was prepared according to general procedure E as a white solid (23 mg, 37%) as a white solid. MS (ESI) m/z 406 [M+H]⁺. ¹H NMR (400 MHz, DMSO-d₆) δ 11.66 (s, 1H), 8.48 (d, J=2.5 Hz, 1H), 7.72 (dd, J=8.4, 2.6 Hz, 1H), 7.64 (d, J=7.4 Hz, 1H), 7.53 (t, J=7.4 Hz, 1H), 7.45 (t, J=7.4 Hz, 1H), 7.23 (d, J=7.6 Hz, 1H), 7.14 (s, 1H), 7.04-6.98 (m, 3H), 5.10 (d, J=15.3 Hz, 1H), 4.92 (t, J=5.6 Hz, 1H), 4.45 (d, J=15.3 Hz, 1H), 3.51 (dd, J=1 4.2, 4.8 Hz, 1H), 3.28-3.22 (m, 1H).

### 2-((2-(3,5-difluoro-4-hydroxybenzyl)-3-oxoisoindolin-1-yl)methyl)benzonitrile (197)

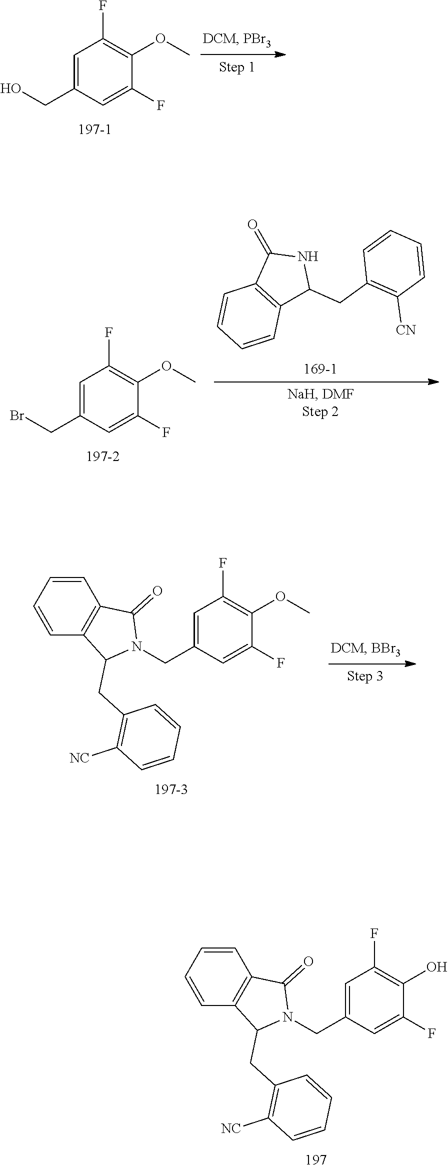 [SMILES: COc1c(F)cc(CO)cc1F ; COc1c(F)cc(CBr)cc1F ; N#Cc1ccccc1CC1NC(=O)c2ccccc21 ; COc1c(F)cc(CN2C(=O)c3ccccc3C2Cc2ccccc2C#N)cc1F ; N#Cc1ccccc1CC1c2ccccc2C(=O)N1Cc1cc(F)c(O)c(F)c1]

Step 1 was performed according to the procedure outlined for step 1 for the preparation of compound 195.

Step 2 was performed according to the procedure outlined for step 1 in general procedure A.

Step 3 was performed according to the procedure outlined for step 2 for the preparation of compound 12 to obtain compound 197 as a white solid (24 mg, 34%). MS (ESI) m/z 391 [M+H]⁺. ¹H NMR (400 MHz, DMSO-d₆) δ 10.16 (s, 1H), 7.72 (d, J=7.7 Hz, 1H), 7.65 (d, J=7.4 Hz, 1H), 7.61-7.37 (m, 4H), 7.30 (d, J=7.8 Hz, 1H), 7.12 (d, J=7.5 Hz, 1H), 6.87 (d, J=7.7 Hz, 2H), 5.00 (d, J=15.4 Hz, 1H), 4.93 (t, J=5.9 Hz, 1H), 4.41 (d, J=15.4 Hz, 1H), 3.59 (dd, J=14.3, 5.2 Hz, 1H), 3.26 (dd, J=14.3, 6.6 Hz, 1H).

### rel-(S)-6-((3-methyl-5-(oxetan-3-yl)-1-oxoisoindolin-2-yl)methyl)benzo[d]oxazol-2(3H)-one (198)

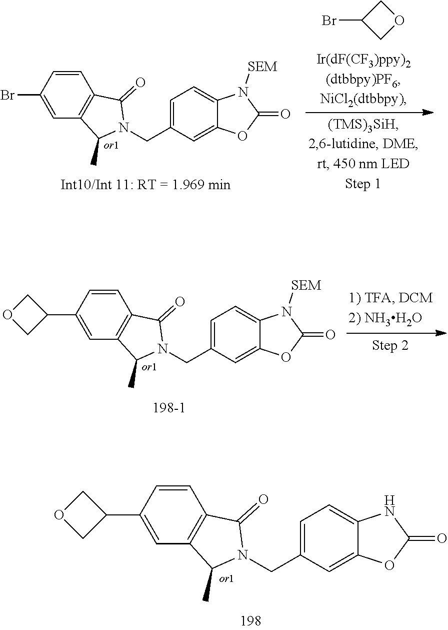 [SMILES: BrC1COC1 ; C[C@H]1c2cc(Br)ccc2C(=O)N1Cc1ccc2c(c1)oc(=O)n2COCC[Si](C)(C)C ; C[C@H]1c2cc(C3COC3)ccc2C(=O)N1Cc1ccc2c(c1)oc(=O)n2COCC[Si](C)(C)C ; C[C@H]1c2cc(C3COC3)ccc2C(=O)N1Cc1ccc2[nH]c(=O)oc2c1]

### Step 1: rel-(S)-6-((3-methyl-5-(oxetan-3-yl)-1-oxoisoindolin-2-yl)methyl)-3-((2-(trimethylsilyl)ethoxy)methyl)benzo[d]oxazol-2(3H)-one (198-1)

To an 8 mL vial equipped with a stir bar was added photocatalyst Ir[dF(CF₃)ppy]₂(dtbbpy)PF₆ (1 mg, 10 μmol), the stereoisomer of Int 10 or Int 11 that has RT of 1.969 min (100 mg, 0.2 mmol), 3-bromooxetane (27 mg, 0.2 mmol), tris(trimethylsilyl)silane (103 mg, 0.4 mmol). The vial was sealed and placed N₂ before 4 mL of DME was added. To a separate vial was added NiCl₂(dtbbpy) (4 mg, 10 μmol) and 2,6-lutidine(44 mg, 0.41 mmol). The catalyst vial was sealed, purged with nitrogen then to it was added 2 mL of DME. The precatalyst solution was sonicated or stirred for 5 min, after which, 1 mL (0.5 mol % catalyst) was syringed into the reaction vessel. The solution was degassed by sparging with nitrogen while stirring for 10 minutes before sealing with Parafilm. The reaction was stirred and irradiated with a 34 W blue LED lamp (7 cm away, with cooling fan to keep the reaction temperature at 25° C. for 12 h. The resulting mixture was diluted with water (5 mL). The resulted mixture was diluted with EtOAc (30 mL), the solution was successively washed with water (30 mL) and brine (30 mL), dried over anhydrous Na₂SO₄, after filtration, the filtrate was concentrated under reduced pressure. The residue was purified by TLC, Layered with (PE:EtOAc) (1:1) to afford the title compound (60 mg, 31%) as a white solid. MS (ESI) m/z 467 [M+H]⁺.

### Step 2: rel-(S)-6-((3-methyl-5-(oxetan-3-yl)-1-oxoisoindolin-2-yl)methyl)benzo[d]oxazol-2(3H)-one (198)

To a solution of 198-1 (60 mg, 0.1 mmol) in DCM (5 mL) was added dropwise TFA (2 mL) at 0° C. The resulting mixture was stirred for additional 0.5 h at 0° C. LC-MS and TLC showed 198-1 was disappeared. The resulting mixture was concentrated under reduced pressure. NH₃·H₂O (5 mL) was dropwise added in mixture under 0° C. The resulting mixture was stirred for additional 0.5 h at 0° C. The mixture was concentrated under reduced pressure. The residue was purified by TLC, Layered with (DCM:MeOH) (50:1) to afford the title compound (19 mg, 43%) as a white solid. MS (ESI) m/z 320 [M+H]⁺. ¹H NMR (400 MHz, CDCl₃) δ 7.88 (d, J=7.8 Hz, 1H), 7.51-7.44 (m, 2H), 7.16 (s, 1H), 7.11 (d, J=8.1 Hz, 1H), 6.97 (d, J=8.0 Hz, 1H), 5.28 (d, J=15.4 Hz, 1H), 5.15-5.10 (m, 2H), 4.78-4.74 (m, 2H), 4.41 (q, J=6.6 Hz, 1H), 4.36-4.26 (m, 2H), 1.47 (d, J=6.7 Hz, 3H).

### 3-fluoro-4-((3-oxo-2-((2-oxo-2,3-dihydrobenzo[d]oxazol-6-yl)methyl)isoindolin-1-yl)methyl)benzonitrile (199)

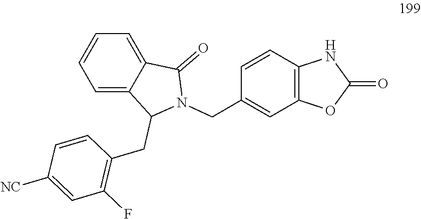 [SMILES: N#Cc1ccc(CC2c3ccccc3C(=O)N2Cc2ccc3[nH]c(=O)oc3c2)c(F)c1]

The title compound 199 was prepared according to general procedure E as a white solid (25 mg, 27.5%). MS (ESI) m/z 414 [M+H]⁺. ¹H NMR (400 MHz, DMSO-d₆) δ 11.66 (s, 1H), 7.69 (d, J=10.0 Hz, 1H), 7.60 (d, J=7.6 Hz, 1H), 7.55-7.50 (m, 2H), 7.44 (t, J=7.4 Hz, 1H), 7.31 (d, J=7.6 Hz, 1H), 7.25-7.20 (m, 2H), 7.04 (s, 2H), 5.17 (d, J=15.2 Hz, 1H), 4.79 (d, J=5.6 Hz, 1H), 4.48 (d, J=15.2 Hz, 1H), 3.53 (dd, J=14.2, 4.4 Hz, 1H), 3.29-3.25 (m, 1H).

### 3-bromo-2-((3-oxo-2-((2-oxo-2,3-dihydrobenzo[d]oxazol-6-yl)methyl)isoindolin-1-yl) methyl)benzonitrile (200)

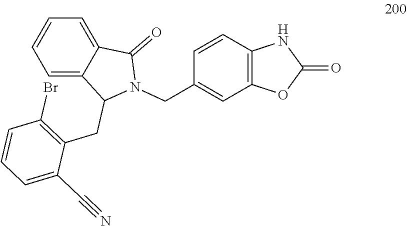 [SMILES: N#Cc1cccc(Br)c1CC1c2ccccc2C(=O)N1Cc1ccc2[nH]c(=O)oc2c1]

The title compound 200 was prepared according to general procedure E as a white solid (80 mg, 47.3%). MS (ESI) m/z 474 [M+H]⁺. ¹H NMR (400 MHz, DMSO-d(,) δ 11.64 (s, 1H), 8.05 (dd, J=8.1, 1.2 Hz, 1H), 7.92 (dd, J=7.7, 1.2 Hz, 1H), 7.80 (d, J=7.4 Hz, 1H), 7.56-7.41 (m, 3H), 7.10 (d, J=1.5 Hz, 1H), 7.06-6.95 (m, 2H), 6.50 (d, J=7.6 Hz, 1H), 5.19 (d, J=15.4 Hz, 1H), 4.84 (dd, J=9.0, 6.4 Hz, 1H), 4.35 (d, J=15.4 Hz, 1H), 3.77 (dd, J=13.6, 6.4 Hz, 1H), 2.95 (dd, J=13.6, 9.0 Hz, 1H).

### 3-((3-oxo-2-((2-oxo-2,3-dihydrobenzo[d]oxazol-6-yl)methyl)isoindolin-1-yl)methyl) picolinonitrile (201)

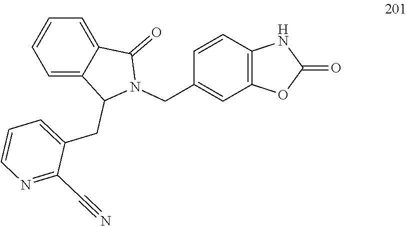 [SMILES: N#Cc1ncccc1CC1c2ccccc2C(=O)N1Cc1ccc2[nH]c(=O)oc2c1]

The title compound 201 was prepared according to general procedure E as a white solid (83 mg, 63.5%). MS (ESI) m/z 397 [M+H]⁺. ¹H NMR (400 MHz, DMSO-d₆) δ 11.61 (s, 1H), 8.54 (dd, J=4.7, 1.5 Hz, 1H), 7.70-7.51 (m, 4H), 7.45 (t, J=7.4 Hz, 1H), 7.30 (d, J=7.6 Hz, 1H), 7.18 (s, 1H), 7.03 (s, 2H), 5.14 (d, J=15.2 Hz, 1H), 4.95 (t, J=5.2 Hz, 1H), 4.56 (d, J=15.2 Hz, 1H), 3.61 (dd, J=14.6, 4.8 Hz, 1H), 3.43 (dd, J=14.6, 6.0 Hz, 1H).

### 4-fluoro-3-((3-oxo-2-((2-oxo-2,3-dihydrobenzo[d]oxazol-6-yl)methyl)isoindolin-1-yl) methyl)benzonitrile (202)

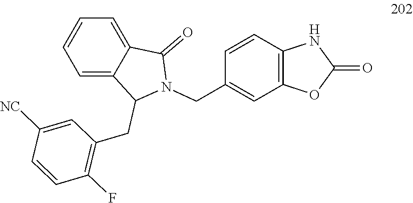 [SMILES: N#Cc1ccc(F)c(CC2c3ccccc3C(=O)N2Cc2ccc3[nH]c(=O)oc3c2)c1]

The title compound 202 was prepared according to general procedure E as a white solid (28 mg, 33%). MS (EST) m/z 414 [M+H]⁺. ¹H NMR (400 MHz, DMSO-d₆) δ 11.62 (s, 1H), 7.76-7.69 (m, 1H), 7.63-7.52 (m, 2H), 7.51-7.41 (m, 2H), 7.37-7.25 (m, 2H), 7.19 (s, L H), 7.04 (s, 2H), 5.12 (d, J=15.2 Hz, 1H), 4.81 (t, J=5.4 Hz, 1H), 4.50 (d, J=15.2 Hz, 1H), 3.45 (dd, J=14.2, 4.6 Hz, 1H), 3.31-3.25 (m, 1H).

### 3-((3-oxo-2-((2-oxo-2,3-dihydrobenzo[d]oxazol-6-yl)methyl)isoindolin-1-yl)methyl) isonicotinonitrile (203)

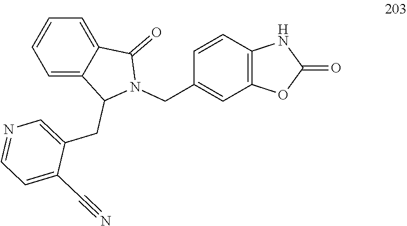 [SMILES: N#Cc1ccncc1CC1c2ccccc2C(=O)N1Cc1ccc2[nH]c(=O)oc2c1]

The title compound 203 was prepared according to general procedure E as a white solid (12 mg, 7.4%). MS (ESI) m/z 397 [M+H]⁺. ¹H NMR (400 MHz, DMSO-d₆) δ 11.62 (s, 1H), 8.58 (d, J=5.0 Hz, 1H), 8.45 (s, 1H), 7.70 (d, J=5.0 Hz, 1H), 7.61 (d, J=7.6 Hz, 1H), 7.55 (td, J=7.5, 1.3 Hz, 1H), 7.45 (t, J=7.4 Hz, 1H), 7.30 (d, J=7.6 Hz, 1H), 7.19 (s, 1H), 7.06-7.02 (m, 2H), 5.17 (d, J=15.2 Hz, 1H), 4.93 (t, J=5.4 Hz, 1H), 4.56 (d, J=15.2 Hz, 1H), 3.61 (dd, J=14.6, 4.8 Hz, 1H), 3.43 (dd, J=14.6, 6.0 Hz, 1H).

### 2-fluoro-3-((3-oxo-2-((2-oxo-2,3-dihydrobenzo[d]oxazol-6-yl)methyl)isoindolin-1-yl) methyl)benzonitrile (204)

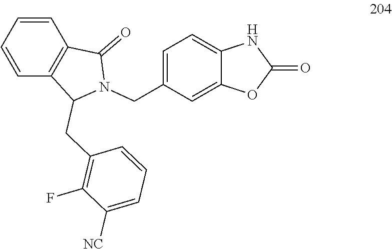 [SMILES: N#Cc1cccc(CC2c3ccccc3C(=O)N2Cc2ccc3[nH]c(=O)oc3c2)c1F]

The title compound 204 was prepared according to general procedure E as a white solid (89 mg, 55.7%). MS (ESI) m/z 414 [M+H]⁺. 1H NMR (400 MHz, DMSO-d₆) δ 11.63 (s, 1H), 7.71 (t, J=7.8 Hz, 1H), 7.63-7.53 (m, 2H), 7.48-7.34 (m, 3H), 7.25-7.15 (m, 2H), 7.04 (brs, 2H), 5.13 (d, J=15.2 Hz, 1H), 4.82 (t, J=5.1 Hz, 1H), 4.50 (d, J=15.2 Hz, 1H), 3.48 (dd, J=14.4, 4.6 Hz, 1H), 3.36 (s, 1H).

### 6-((1-((3-bromopyridin-2-yl)methyl)-3-oxoisoindolin-2-yl)methyl)benzo[d]oxazol-2(3H)-one (205)

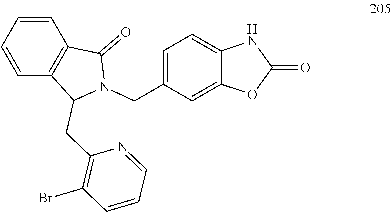 [SMILES: O=C1c2ccccc2C(Cc2ncccc2Br)N1Cc1ccc2[nH]c(=O)oc2c1]

The title compound 205 was prepared according to general procedure E as a white solid (90 mg, 30.5%). MS (ESI) m/z 450 [M+H]⁺. ¹H NMR (400 MHz, DMSO-d₆) δ 11.61 (s, 1H), 8.55 (dd, J=4.6, 1.5 Hz, 11H), 8.02 (dd, J=8.1, 1.5 Hz, 11H), 7.78-7.69 (m, 1H), 7.55-7.44 (m, 2H), 7.28 (dd, J=8.1, 4.6 Hz, 11H), 7.09-7.01 (m, 1H), 7.00-6.94 (m, 2H), 6.88 (dd, J=8.0, 1.5 Hz, 1H), 5.16 (t, J=6.5 Hz, 11H), 5.02 (d, J=15.4 Hz, 11H), 4.44 (d, J=15.4 Hz, 1H), 3.57 (dd, J=15.1, 6.0 Hz, 11H), 3.21 (dd, J=15.1, 7.1 Hz, 1H).

### (R)-6-((1-((3-bromopyridin-2-yl)methyl)-3-oxoisoindolin-2-yl)methyl)benzo[d]oxazol-2(3H)-one (206) and (S)-6-((1-((3-bromopyridin-2-yl)methyl)-3-oxoisoindolin-2-yl)methyl)benzo[d]oxazol-2(3H)-one (207)

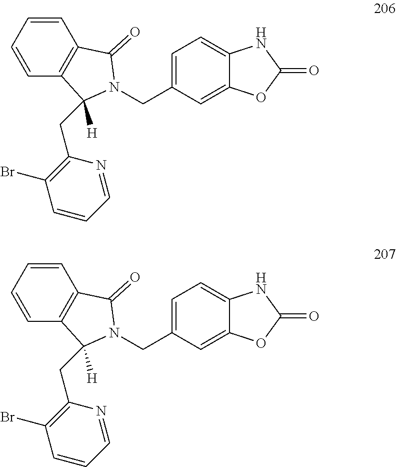 [SMILES: O=C1c2ccccc2[C@@H](Cc2ncccc2Br)N1Cc1ccc2[nH]c(=O)oc2c1 ; O=C1c2ccccc2[C@H](Cc2ncccc2Br)N1Cc1ccc2[nH]c(=O)oc2c1]

Chiral separation of 205 by chiral prep-HPLC (column: CHIRALPAK IF; Column size: 2 cm×25 cm, 5 um; Mobile phase A: MtBE; Mobile phase B: MeOH:DCM) was conducted to afford two enantiomers: compound 206 and compound 207. One of the two enantiomers was a white solid, with RT=1.087 min (CHIRAL HPLC, column: CHIRALPAK IF-3; column size: 4.6*50 mm, 3 μm; mobile phase: MTBE (0.1% DEA):(MeOH:DCM=1:1)=50:50; flow: 1.0 mL/min). MS (ESI) m/z 450.1 [M+H]⁺. 11H NMR (400 MHz, DMSO-d₆) δ 11.61 (s, 1H), 8.54 (dd, J=4.7, 1.7 Hz, 1H), 8.02 (dd, J=8.1, 1.6 Hz, 1H), 7.78-7.69 (m, 1H), 7.55-7.44 (m, 2H), 7.28 (dd, J=8.1, 4.6 Hz, 1H), 7.09-7.01 (m, 11H), 7.00-6.94 (m, 2H), 6.88 (dd, J=7.9, 1.7 Hz, 11H), 5.16 (t, J=6.5 Hz, 1H), 5.02 (d, J=15.4 Hz, 1H), 4.44 (d, J=15.4 Hz, 1H), 3.57 (dd, J=15.1, 6.0 Hz, 1H), 3.21 (dd, J=15.1, 7.1 Hz, 1H). The other one of the two enantiomers was a white solid, with RT=1.302 min (CHIRAL HPLC, column: CHIRALPAK IF-3; column size: 4.6*50 mm, 3 9 m; mobile phase: MTBE (0.1% DEA):(MeOH:DCM=1:1)=50:50; flow: 1.0 mL/min). MS (ESI) m/z 450.1 [M+H]⁺. ¹H NMR (400 MHz, DMSO-d₆) δ 11.61 (s, 1H), 8.54 (dd, J=4.7, 1.4 Hz, 1H), 8.02 (dd, J=8.1, 1.5 Hz, 1H), 7.78-7.69 (m, 1H), 7.55-7.44 (m, 2H), 7.28 (dd, J=8.1, 4.6 Hz, 1H), 7.09-7.01 (m, 1H), 7.00-6.94 (m, 2H), 6.88 (d, J=8.0 Hz, 1H), 5.16 (t, J=6.5 Hz, 1H), 5.02 (d, J=15.4 Hz, 1H), 4.44 (d, J=15.4 Hz, 1H), 3.57 (dd, J=15.1, 6.0 Hz, 1H), 3.21 (dd, J=15.1, 7.1 Hz, 1H).

### 6-((1-(2-aminobenzyl)-3-oxoisoindolin-2-yl)methyl)benzo[d]oxazol-2(3H)-one (208)

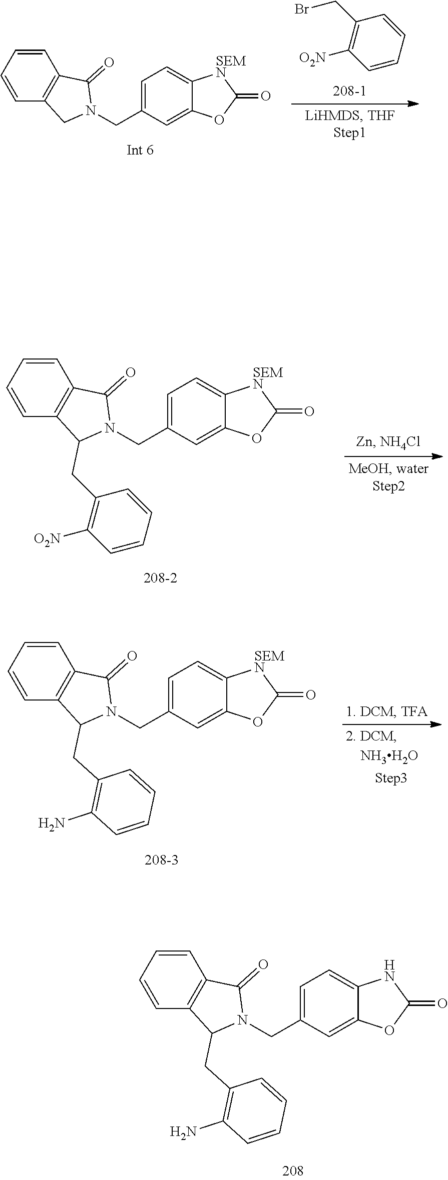 [SMILES: C[Si](C)(C)CCOCn1c(=O)oc2cc(CN3Cc4ccccc4C3=O)ccc21 ; O=[N+]([O-])c1ccccc1CBr ; C[Si](C)(C)CCOCn1c(=O)oc2cc(CN3C(=O)c4ccccc4C3Cc3ccccc3[N+](=O)[O-])ccc21 ; C[Si](C)(C)CCOCn1c(=O)oc2cc(CN3C(=O)c4ccccc4C3Cc3ccccc3N)ccc21 ; Nc1ccccc1CC1c2ccccc2C(=O)N1Cc1ccc2[nH]c(=O)oc2c1]

### Step 1. 6-((1-(2-nitrobenzyl)-3-oxoisoindolin-2-yl)methyl)-3-((2-(trimethylsilyl)ethoxy) methyl)benzo[d]oxazol-2(3H)-one (208-2)

To a solution of 6-((1-oxoisoindolin-2-yl)methyl)-3-((2-(trimethylsilyl)ethoxy)methyl) benzo[d]oxazol-2(3H)-one (300 mg, 0.73 mmol) in THF (3 mL) was added LiHMDS (0.8 mL, 0.8 mmol) at −78° C. under N₂. The mixture was stirred for 30 min. 1-(bromomethyl)-2-nitrobenzene (158 mg, 0.73 mmol) was added. This suspension was allowed to stir for an additional 1 h. NH₄Cl (aq.) was added and then extracted with EtOAc. The organic layer was separated, dried over anhydrous Na₂SO₄ and concentrated under vacuum. The residue was purified by prep TLC (DCM/MeOH=25/1) to give 6-((1-(2-nitrobenzyl)-3-oxoisoindolin-2-yl)methyl)-3-((2-(trimethylsilyl)ethoxy)methyl)benzo[d]oxazol-2(3H)-one (300 mg, 75%) as a yellow solid. MS (ESI) m/z 546 [M+H]⁺.

### Step 2. 6-((1-(2-aminobenzyl)-3-oxoisoindolin-2-yl)methyl)-3-((2-(trimethylsilyl)ethoxy) methyl)benzo[d]oxazol-2(3H)-one (208-3)

To a solution of 6-((1-(2-nitrobenzyl)-3-oxoisoindolin-2-yl)methyl)-3-((2-(trimethylsilyl) ethoxy)methyl)benzo[d]oxazol-2(3H)-one (300 mg, 0.55 mmol) and NH₄Cl (147 mg, 2.75 mmol) in methanol (5 mL) and water (5 mL) was added Zn (180 mg, 2.75 mmol) under N₂. The mixture was stirred at 80° C. for 2 h, the mixture was diluted with EtOAc, and washed with water. The organic layer was separated, dried over anhydrous Na₂SO₄ and concentrated. The residue was purified by prep TLC (DCM/MeOH=20/1) to give 6-((1-(2-aminobenzyl)-3-oxoisoindolin-2-yl)methyl)-3-((2-(trimethylsilyl)ethoxy)methyl)benzo[d]oxazol-2(3H)-one (200 mg, 70%) as a yellow solid. MS (ESI) m/z 516 [M+H]⁺.

### Step 3. 6-((1-(2-aminobenzyl)-3-oxoisoindolin-2-yl)methyl)benzo[d]oxazol-2(3H)-one (208)

To a solution of 6-((1-(2-aminobenzyl)-3-oxoisoindolin-2-yl)methyl)-3-((2-(trimethylsilyl) ethoxy)methyl)benzo[d]oxazol-2(3H)-one (100 mg, 0.19 mmol) in DCM (3 mL) was added TFA (1 mL). The mixture was stirred for 3 h and then concentrated under vacuum. The residue was taken in DCM (3 mL) and ammonium hydroxide (1 mL) was added at 0′C. This suspension was allowed to stir for an additional 1 h. The mixture was concentrated and purified by prep HPLC to give 6-((1-(2-aminobenzyl)-3-oxoisoindolin-2-yl)methyl)benzo[d]oxazol-2(3H)-one (10 mg, 13%) as a white solid. MS (ESI) m/z 386 [M+H]⁺. ¹H NMR (400 MHz, DMSO-d₆) δ 11.66 (s, 1H), 7.74-7.65 (m, 1H), 7.51-7.41 (m, 2H), 7.11-6.88 (m, 5H), 6.84 (d, J=7.5 Hz, 1H), 6.73-6.67 (m, 1H), 6.56-6.47 (m, 1H), 5. 10 (d, J=15.0 Hz, 1H), 4.97 (s, 2H), 4.75 (t, J=6.9 Hz, 1H), 4.23 (d, J=15.0 Hz, 1H), 3.18 (dd, J=14.1, 6.6 Hz, 1H), 2.78 (dd, J=14.0, 7.3 Hz, 1H).

### 6-((3-((3-bromopyridin-2-yl)methyl)-5-cyclopropyl-1-oxoisoindolin-2-yl)methyl)benzo[d]oxazol-2(3H)-one (209)

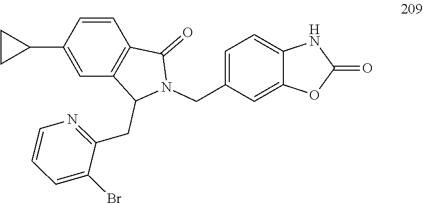 [SMILES: O=C1c2ccc(C3CC3)cc2C(Cc2ncccc2Br)N1Cc1ccc2[nH]c(=O)oc2c1]

The title compound 209 was prepared according to general procedure E as a white solid (12 mg, 14%). MS (ESI) m/z 490 [M+H]⁺. ¹H NMR (400 MHz, DMSO-d₆) δ 11.68 (s, 1H), 8.55 (d, J=4.7 Hz, 1H), 8.03 (d, J=8.0 Hz, 1H), 7.58 (d, J=7.8 Hz, 1H), 7.29 (dd, J=8.1, 4.6 Hz, 1H), 7.23 (d, J=7.9 Hz, 1H), 6.99-6.91 (m, 2H), 6.87-6.80 (m, 1H), 6.56 (s, 1H), 5.06-4.95 (m, 2H), 4.37 (d, J=15.4 Hz, 1H), 3.54 (dd, J=14.8, 6.4 Hz, 1H), 3.18 (dd, J=14.8, 7.2 Hz, 11H), 1.96-1.87 (m, 1H), 0.99-0.91 (m, 2H), 0.65-0.51 (m, 2H).

209-1 was prepared according to the procedure outlined for step 1 for the preparation of compound 181.

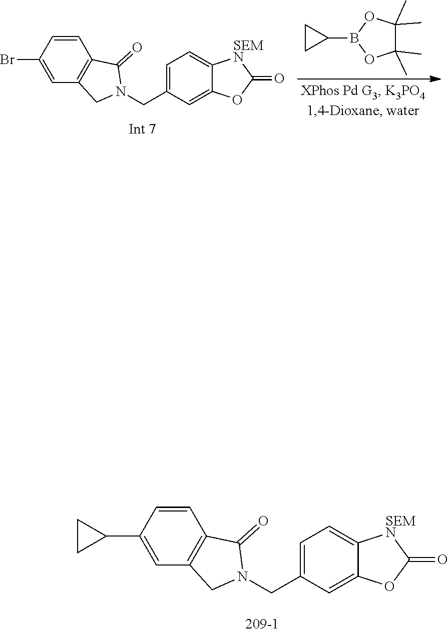 [SMILES: C[Si](C)(C)CCOCn1c(=O)oc2cc(CN3Cc4cc(Br)ccc4C3=O)ccc21 ; CC1(C)OB(C2CC2)OC1(C)C ; C[Si](C)(C)CCOCn1c(=O)oc2cc(CN3Cc4cc(C5CC5)ccc4C3=O)ccc21]

### 6-((1-(2-(difluoromethoxy)benzyl)-3-oxoisoindolin-2-yl)methyl)benzo[d]oxazol-2(3H)-one (210)

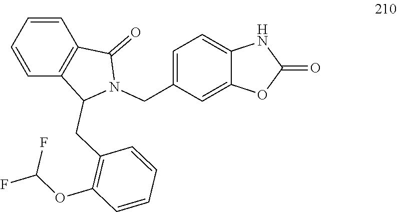 [SMILES: O=C1c2ccccc2C(Cc2ccccc2OC(F)F)N1Cc1ccc2[nH]c(=O)oc2c1]

The title compound 210 was prepared according to general procedure E as a white solid (32 mg, 31%). MS (ESI) m/z 437 [M+H]⁺. ¹H NMR (400 MHz, DMSO-d₆) δ 11.63 (s, 1H), 7.71-7.61 (m, 1H), 7.52-7.40 (m, 2H), 7.36-7.27 (m, 1H), 7.17-6.95 (m, 8H), 5.15 (d, J=15.2 Hz, 1H), 4.71 (dd, J=7.3, 5.1 Hz, 1H), 4.39 (d, J=15.1 Hz, 1H), 3.47 (dd, J=13.9, 5.1 Hz, 1H), 2.94 (dd, J=13.8, 7.4 Hz, 1H).

### 6-((1-((3-bromo-4-fluoropyridin-2-yl)methyl)-3-oxoisoindolin-2-yl)methyl)benzo[d]oxazol-2(3H)-one (2H)

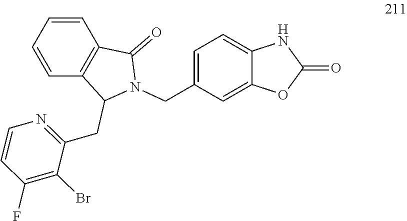 [SMILES: O=C1c2ccccc2C(Cc2nccc(F)c2Br)N1Cc1ccc2[nH]c(=O)oc2c1]

The title compound 211 was prepared according to general procedure E as a white solid (12 mg, 14%). MS (ESI) m/z 468 [M+H]⁺. ¹H NMR (400 MHz, DMSO-dt) δ 11.64 (s, 1H), 8.54 (dd, J=8.0, 5.4 Hz, 1H), 7.78-7.70 (m, 1H), 7.57-7.46 (m, 2H), 7.41 (dd, J=8.5, 5.4 Hz, 1H), 7.18-7.12 (m, 1H), 6.98-6.92 (m, 2H), 6.85 (d, J=7.9 Hz, 1H), 5.21 (t, J=6.4 Hz, 1H), 4.90 (d, J=15.4 Hz, 1H), 4.51 (d, J=15.4 Hz, 1H), 3.55 (dd, J=15.2, 6.4 Hz, 1H), 3.29 (dd, J=15.2, 6.8 Hz, 1H).

The intermediate 211-3 was prepared as follows:

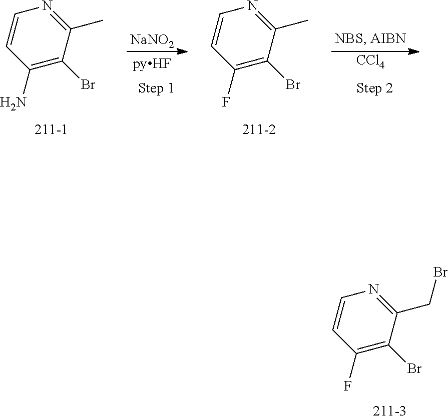

### Step 1: 3-bromo-4-fluoro-2-methylpyridine (211-2)

3-bromo-2-methylpyridin-4-amine (1 g, 5.35 mmol) was taken in pyridine hydrofluoride (7.5 mL) and cooled at −10° C. NaNO₂ (553 mg, 8.02 mmol) was added portion-wise over 30 min. The mixture was allowed to come to r.t. and finally heated at 60° C. for 1 h. The mixture was poured onto crushed ice (ca 50 g). The mixture was neutralized with 1M Na₂CO₃ and extracted with EA (200 mL). The organic layer was further washed with brine, dried over MgSO₄, filtered and the filtrate concentrated. The residue was purified by silica gel column chromatography (PE/EA=1/3) to give 3-bromo-4-fluoro-2-methylpyridine (220 mg, 21%) as a brown oil.

Step 2 was performed according to the procedure outlined for step 3 for the preparation of Int 1.

### 4-chloro-3-((3-oxo-2-((2-oxo-2,3-dihydrobenzo[d]oxazol-6-yl)methyl)isoindolin-1-yl) methyl)picolinonitrile (212)

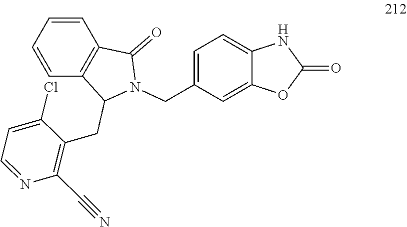 [SMILES: N#Cc1nccc(Cl)c1CC1c2ccccc2C(=O)N1Cc1ccc2[nH]c(=O)oc2c1]

The title compound 212 was prepared according to general procedure E using 4-chloro-3-methylpicolinonitrile as a starting material to afford a white solid (70 mg, 26.5%). MS (ESI) m/z 431 [M+H]⁺. ¹H NMR (400 MHz, DMSO-d₆) δ 11.64 (s, 1H), 8.68 (d, J=5.2 Hz, 1H), 7.98 (d, J=5.2 Hz, 1H), 7.79 (d, J=7.4 Hz, 1H), 7.59-7.42 (m, 2H), 7.19 (s, 1H), 7.05 (s, 2H), 6.59 (d, J=7.5 Hz, 1H), 5.19 (d, J=15.4 Hz, 1H), 4.81 (dd, J=9.2, 5.6 Hz, 1H), 4.45 (d, J=15.4 Hz, 1H), 3.83 (dd, J=14.0, 5.6 Hz, 1H), 2.89 (dd, J=14.0, 9.2 Hz, 1H).

### 6-((1-(4-methoxybenzyl)-3-oxoisoindolin-2-yl)methyl)benzo[d]oxazol-2(3H)-one (213)

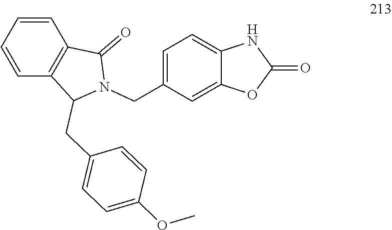 [SMILES: COc1ccc(CC2c3ccccc3C(=O)N2Cc2ccc3[nH]c(=O)oc3c2)cc1]

The title compound 213 was prepared according to general procedure E as a white solid (38 mg, 46.4%). MS (ESI) m/z 401 [M+H]⁺. ¹H NMR (400 MHz, DMSO-d₆) δ 11.62 (s, 1H), 7.62-7.49 (m, 2H), 7.42 (t, J=7.4 Hz, 1H), 7.34 (d, J=7.6 Hz, 1H), 7.21 (s, 1H), 7.11-7.01 (m, 2H), 6.90-6.82 (m, 2H), 6.74-6.66 (m, 2H), 5.14 (d, J=15.1 Hz, 1H), 4.66 (dd, J=6.3, 4.1 Hz, 1H), 4.44 (d, J=15.1 Hz, 1H), 3.66 (s, 3H), 3.30 (dd, J=14.2, 4.1 Hz, 1H), 3.11 (dd, J=14.2, 6.3 Hz, 1H).

### 6-((1-((3-fluoropyridin-2-yl)methyl)-3-oxoisoindolin-2-yl)methyl)benzo[d]oxazol-2(3H)-one (214)

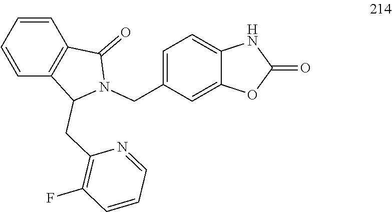 [SMILES: O=C1c2ccccc2C(Cc2ncccc2F)N1Cc1ccc2[nH]c(=O)oc2c1]

The title compound 214 was prepared according to general procedure E as a white solid (14 mg, 11%). MS (ESI) m/z 390 [M+H]⁺. ¹H NMR (400 MHz, DMSO-d₆) δ 11.66 (s, 1H), 8.35-8.32 (m, 1H), 7.68-7.61 (m, 1H), 7.60-7.41 (m, 3H), 7.36-7.29 (m, 1H), 7.15 (d, J=7.4 Hz, 1H), 7.11 (d, J=1.4 Hz, 1H), 7.04-6.94 (m, 2H), 5.11 (d, J=15.4 Hz, 1H), 4.98 (t, J=5.8 Hz, 1H), 4.48 (d, J=15.4 Hz, 1H), 3.60-3.52 (m, 1H), 3.25-3.16 (m, 1H).

### 6-((1-((3-ethynylpyridin-2-yl)methyl)-3-oxoisoindolin-2-yl)methyl)benzo[d]oxazol-2(3H)-one (215)

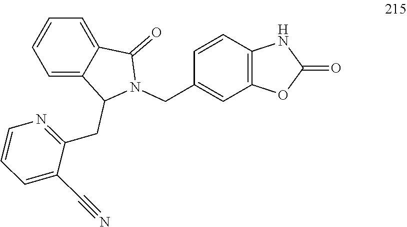 [SMILES: N#Cc1cccnc1CC1c2ccccc2C(=O)N1Cc1ccc2[nH]c(=O)oc2c1]

The title compound 215 was prepared according to the procedure described for compound 181 as a white solid (10 mg, 33%). MS (ESI) m/z 396 [M+H]⁺. ¹H NMR (400 MHz, DMSO-d₆) δ 11.64 (s, 1H), 8.54 (dd, J=4.9, 1.8 Hz, 1H), 7.85 (dd, J=7.9, 1.8 Hz, 1H), 7.75-7.68 (m, 1H), 7.53-7.43 (m, 2H), 7.34 (dd, J=7.8, 4.8 Hz, 1H), 7.01-6.94 (m, 3H), 6.90-6.84 (m, 1H), 5.12 (t, J=6.5 Hz, 1H), 5.06 (d, J=15.4 Hz, 1H), 4.50 (s, 1H), 4.40 (d, J=15.4 Hz, 1H), 3.63 (dd, J=14.6, 6.0 Hz, 1H), 3.26 (dd, J=14.6, 7.1 Hz, 1H).

### 6-((1-(benzo[d]oxazol-6-ylmethyl)-3-oxoisoindolin-2-yl)methyl)benzo[d]oxazol-2(3H)-one (216)

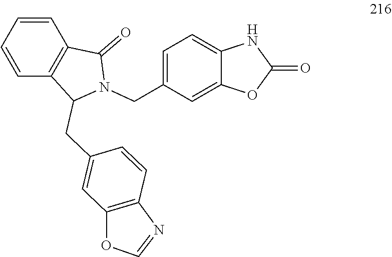 [SMILES: O=C1c2ccccc2C(Cc2ccc3ncoc3c2)N1Cc1ccc2[nH]c(=O)oc2c1]

The title compound 216 was prepared according to general procedure E as a yellow solid (15 mg, 12%). MS (ESI) m/z 412 [M+H]⁺. ¹H NMR (400 MHz, DMSO-d₆) δ 11.62 (s, 1H), 8.63 (s, 1H), 7.61-7.50 (m, 3H), 7.46-7.37 (m, 2H), 7.36-7.31 (m, 1H), 7.22-7.17 (m, 1H), 7.11-6.96 (m, 3H), 5.13 (d, J=15.1 Hz, 11H), 4.85-4.77 (m, 1H), 4.52 (d, J=15.1 Hz, 1H), 3.52 (dd, J=14.2, 4.3 Hz, 1H), 3.37 (dd, J=14.2, 6.1 Hz, 1H).

### 6-((1-((5-methylthiazol-4-yl)methyl)-3-oxoisoindolin-2-yl)methyl)benzo[d]oxazol-2(3H)-one (217)

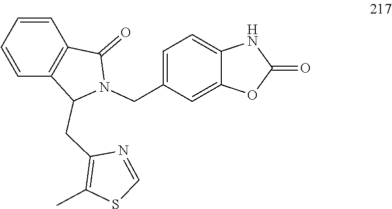 [SMILES: Cc1scnc1CC1c2ccccc2C(=O)N1Cc1ccc2[nH]c(=O)oc2c1]

The title compound 217 was prepared according to general procedure E as a light yellow solid (48 mg, 30%). MS (ESI) m/z 392 [M+H]⁺. ¹H NMR (400 MHz, DMSO-d₆) δ 11.60 (s, 1H), 8.83 (s, 1H), 7.67 (d, J=6.7 Hz, 1H), 7.54-7.42 (m, 2H), 7.14 (d, J=1.4 Hz, 1H), 7.09 (d, J=7.3 Hz, 1H), 7.06-6.96 (m, 2H), 5.12 (d, J=15.3 Hz, 1H), 4.83 (t, J=6.5 Hz, 1H), 4.42 (d, J=15.3 Hz, 11H), 3.40 (dd, J=14.5, 5.1 Hz, 1H), 3.07 (dd, J=14.5, 7.1 Hz, 1H), 2.12 (s, 3H).

The intermediate 217-3 was prepared as follows:

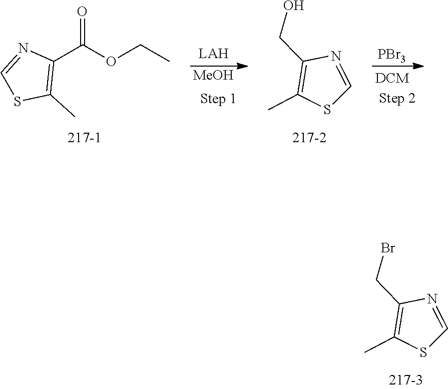

### Step 1. (5-methylthiazol-4-yl)methanol (217-2)

To a stirred solution of ethyl 5-methylthiazole-4-carboxylate (200 mg, 1.17 mmol) in THF (5 mL) at 0° C. was added LiAlH₄ (89 mg, 2.34 mmol). After 30 min, the reaction mixture was carefully quenched with water, NaOH (aq.). The solution was filtered, and the filtrate was concentrated to give (5-methylthiazol-4-yl)methanol (74 mg) as a colorless oil, which was used in the next step without further purification. MS (ESI) m/z 129 [M+H]⁺.

### Step 2. 4-(bromomethyl)-5-methylthiazole (217-3)

To a 0° C. mixture of (5-methylthiazol-4-yl)methanol (74 mg, 0.57 mmol) in DCM (3 mL) was added PBr₃ (93 mg, 0.34 mmol) dropwise. The resulting mixture was allowed to warm to r.t. and stirred for 1.5 h. The reaction mixture was adjusted to pH 7 using NaHCO₃ (aq.). The aqueous layer was separated, and the organic layer was washed with brine, dried over anhydrous Na₂SO₄ and concentrated under reduced pressure to give 4-(bromomethyl)-5-methylthiazole (110 mg) as a yellow solid, which was used in the next step without any further purification. MS (ESI) m/z 192 [M+H]H⁺.

### 6-((1-(benzo[d]oxazol-7-ylmethyl)-3-oxoisoindolin-2-yl)methyl)benzo[d]oxazol-2(3H)-one (218)

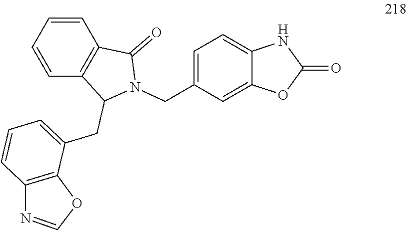 [SMILES: O=C1c2ccccc2C(Cc2cccc3ncoc23)N1Cc1ccc2[nH]c(=O)oc2c1]

The title compound 218 was prepared according to general procedure E as a white solid (10 mg, 5%). MS (ESI) m/z 412 [M+H]⁺. ¹H NMR (400 MHz, DMSO-d₆) δ 11.61 (s, 1H), 8.62 (s, 1H), 7.58 (t, J=8.1 Hz, 2H), 7.48 (t, J=7.5 Hz, 1H), 7.40 (t, J=7.4 Hz, 1H), 7.31 (d, J=7.6 Hz, 1H), 7.19 (t, J=7.8 Hz, 1H), 7.10 (s, 1H), 7.04-6.95 (m, 3H), 5.14 (d, J=15.2 Hz, 1H), 4.91 (t, J=5.4 Hz, 1H), 4.47 (d, J=15.2 Hz, 1H), 3.69-3.48 (m, 2H).

### 6-((3-oxo-2-((2-oxo-2,3-dihydrobenzo[d]oxazol-6-yl)methyl)isoindolin-1-yl)methyl)picolinonitrile (219)

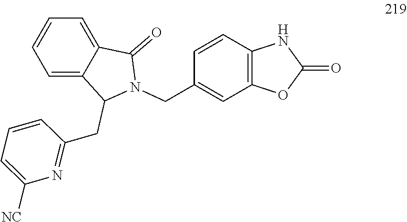 [SMILES: N#Cc1cccc(CC2c3ccccc3C(=O)N2Cc2ccc3[nH]c(=O)oc3c2)n1]

The title compound 219 was prepared according to general procedure E as a white solid (6 mg, 5.4%). MS (ESI) m/z 397 [M+H]⁺. ¹H NMR (400 MHz, DMSO-d₆) δ 11.62 (s, 1H), 8.58 (d, J=5.0 Hz, 1H), 8.45 (s, 1H), 7.70 (d, J=5.0 Hz, 1H), 7.64-7.51 (m, 2H), 7.45 (t, J=7.4 Hz, 1H), 7.30 (d, J=7.5 Hz, 1H), 7.19 (s, 1H), 7.04 (d, J=2.1 Hz, 2H), 5.17 (d, J=15.2 Hz, 1H), 4.93 (t, J=5.3 Hz, 1H), 4.56 (d, J=15.3 Hz, 1H), 3.61 (dd, J=14.5, 4.8 Hz, 1H), 3.43 (dd, J=14.6, 5.9 Hz, 1H).

### 6-((1-((4-chloropyridin-2-yl)methyl)-3-oxoisoindolin-2-yl)methyl)benzo[d]oxazol-2(3H)-one (220)

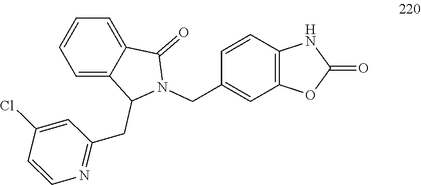 [SMILES: O=C1c2ccccc2C(Cc2cc(Cl)ccn2)N1Cc1ccc2[nH]c(=O)oc2c1]

The title compound 220 was prepared according to general procedure E as a white solid (51 mg, 50%). MS (ESI) m/z 406 [M+H]⁺. ¹H NMR (400 MHz, DMSO-d₆) δ 11.63 (s, 1H), 7.86-7.79 (m, 2H), 7.64 (d, J=7.5 Hz, 1H), 7.56 (t, J=7.5 Hz, 1H), 7.46 (t, J=7.4 Hz, 1H), 7.40-7.29 (m, 2H), 7.12-7.07 (m, 1H), 7.06-6.92 (m, 2H), 5.08 (d, J=15.4 Hz, 1H), 4.99 (t, J=5.5 Hz, 1H), 4.42 (d, J=15.4 Hz, 1H), 3.52 (dd, J=14.4, 5.1 Hz, 1H), 3.43 (dd, J=14.4, 5.9 Hz, 1H).

### 5-bromo-6-((3-oxo-2-((2-oxo-2,3-dihydrobenzo[d]oxazol-6-yl)methyl)isoindolin-1-yl) methyl)picolinonitrile (221)

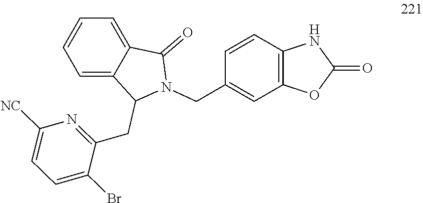 [SMILES: N#Cc1ccc(Br)c(CC2c3ccccc3C(=O)N2Cc2ccc3[nH]c(=O)oc3c2)n1]

The title compound 221 was prepared according to general procedure E as a white solid (5 mg, 3.8%). MS (ESI) m/z 475 [M+H]⁺. ¹H NMR (400 MHz, DMSO-d₆) δ 11.66 (s, 1H), 8.27 (d, J=8.2 Hz, 1H), 7.85 (d, J=8.2 Hz, 1H), 7.74 (dd, J=6.4, 2.8 Hz, 1H), 7.57-7.49 (m, 2H), 7.29 (d, J=7.2 Hz, 1H), 6.92-6.95 (m, 2H), 6.84-6.81 (m, 11H), 5.18 (t, J=6.0 Hz, 1H), 4.94 (d, J=15.6 Hz, 1H), 4.42 (d, J=15.6 Hz, 1H), 3.57 (dd, J=15.8, 6.2 Hz, 1H), 3.37 (dd, J=15.8, 6.2 Hz, 1H).

### 6-((1-((3-bromopyridin-2-yl)methyl)-3-oxoisoindolin-2-yl)methyl)-5-fluorobenzo[d]oxazol-2(3H)-one (223)

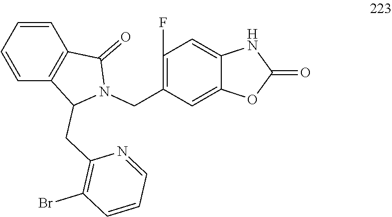 [SMILES: O=C1c2ccccc2C(Cc2ncccc2Br)N1Cc1cc2oc(=O)[nH]c2cc1F]

The title compound 223 was prepared according to general procedure F as a white solid (22 mg, 25.5%). MS (ESI) m/z 468 [M+H]⁺. ¹H NMR (400 MHz, CDCl₃) δ 8.50 (dd, J=4.8, 1.6 Hz, 1H), 7.90 (dd, J=6.0, 2.4 Hz, 1H), 7.83 (dd, J=8.0, 1.6 Hz, 1H), 7.51-7.41 (m, 2H), 7.11 (dd, J=8.0, 4.8 Hz, 1H), 7.03 (d, J=6.0 Hz, 2H), 6.68 (d, J=8.8 Hz, 1H), 5.28 (t, J=6.8 Hz, 1H), 5.14 (d, J=15.6 Hz, 1H), 4.58 (d, J=15.6 Hz, 1H), 3.59 (dd, J=15.2, 6.0 Hz, 1H), 3.28 (dd, J=15.2, 7.2 Hz, 1H).

The intermediate 223-5 was prepared as follows:

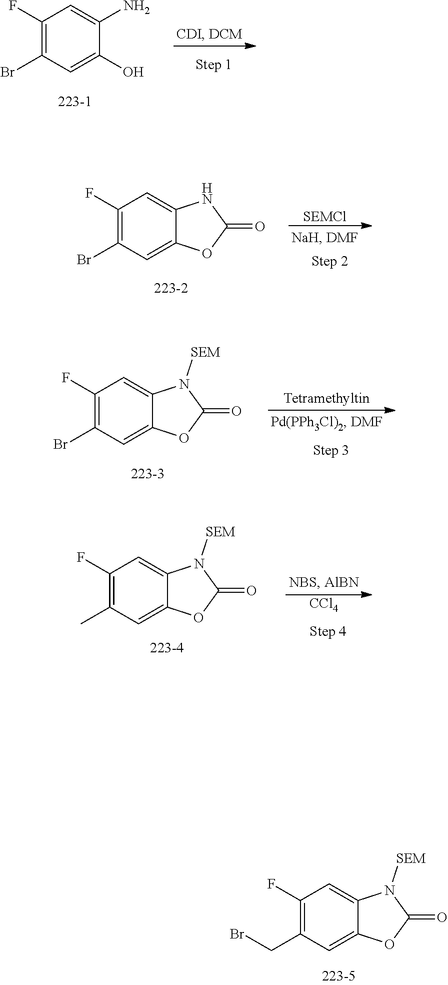

### Step 1. 6-bromo-5-fluoro-3H-1,3-benzoxazol-2-one (223-2)

To a solution of 2-amino-5-bromo-4-fluoro-phenol (2 g, 9.7 mmol) and CDI (1.88 g, 11.64 mmol) in THE (20 mL), TEA (1.3 mL, 19.42 mmol) was added at r.t. under N₂. After addition, the mixture was stirred at 80° C. for 1 h. LC-MS showed the starting material was mostly converted to the product. Then the reaction mixture was added water (50 mL) and extracted with EtOAc (20 mL×3). The organics were then combined and dried (Na₂SO₄) before concentration to dryness. The crude was purified with flash (PE/EtOAc=4:1) to give the product 6-bromo-5-fluoro-3H-1,3-benzoxazol-2-one (2 g, 88.79%) as a yellow oil. MS (ESI) m/z 232 [M+H]⁺.

### Step 2. 2-[(6-bromo-5-fluoro-1,3-benzoxazol-2-yl)oxymethoxy]ethyl-trimethyl-silane (223-3)

To a solution of 6-bromo-5-fluoro-3H-1,3-benzoxazol-2-one (1.1 g, 4.74 mmol) in DMF (11 mL), was added NaH (228 mg, 5.69 mmol) at 0° C. under N₂. After addition, the mixture was stirred at 0° C. for 1 h. Then SEMCl (869 mg, 5.22 mmol) was added. After addition, the mixture was stirred at 0° C. for 1 h. LC-MS showed the starting material was mostly converted to the product. Then the reaction mixture was added water (30 mL) and extracted with EtOAc (15 mL×3). The organics were then combined and dried (Na₂SO₄) before concentration to dryness. The crude was purified with flash (PE/EtOAc=5:1) to give 2-[(6-bromo-5-fluoro-1,3-benzoxazol-2-yl)oxymethoxy]ethyl-trimethyl-silane (1 g, 58.2%) as a yellow oil. MS (ESI) m/z 362 [M+H]⁺.

### Step 3. 5-fluoro-6-methyl-3-(2-trimethylsilylethoxymethyl)-1,3-benzoxazol-2-one (223-4)

To a solution of 6-bromo-5-fluoro-3-(2-trimethylsilylethoxymethyl)-1,3-benzoxazol-2-one (1.1 g, 3.04 mmol), Bis(triphenylphosphine) palladium (II) chloride (426 mg, 0.61 mmol), tetramethyltin (1.01 g, 6.07 mmol) in DMF (10 mL) was added at r.t. under N₂. After addition, the mixture was stirred at 160° C. with microwave for 30 min. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with flash (PE/EtOAc=5:1) to give 5-fluoro-6-methyl-3-(2-trimethylsilylethoxymethyl)-1,3-benzoxazol-2-one (600 mg, 66.4%) as a yellow oil. MS (ESI) m/z 298 [M+H]⁺.

### Step 4. 6-(bromomethyl)-5-fluoro-3-(2-trimethylsilylethoxymethyl)-1,3-benzoxazol-2-one (223-5)

To a solution of 5-fluoro-6-methyl-3-(2-trimethylsilylethoxymethyl)-1,3-benzoxazol-2-one (500 mg, 1.68 mmol) and NBS (329 mg, 1.85 mmol) in CCl₄ (5.0 mL) was added AIBN (28 mg, 0.17 mmol) at r.t. under N₂. After addition, the mixture was stirred at 87° C. with microwave for 1 h. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with flash (PE/EtOAc=3:1) to give 6-(bromomethyl)-5-fluoro-3-(2-trimethylsilylethoxymethyl)-1,3-benzoxazol-2-one (200 mg, 31.6%) as a yellow oil. MS (ESI) m/z 376 [M+H]⁺.

### 6-((1-((3-bromopyridin-2-yl)methyl)-3-oxoisoindolin-2-yl)methyl)-7-fluorobenzo[d]oxazol-2(3H)-one (224)

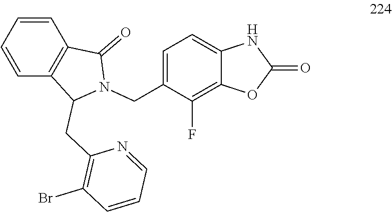 [SMILES: O=C1c2ccccc2C(Cc2ncccc2Br)N1Cc1ccc2[nH]c(=O)oc2c1F]

The title compound 224 was prepared according to the procedure described for compound 223 as a white solid (30 mg, 30.4%). MS (ESI) m/z 468 [M+H]⁺. ¹H NMR (400 MHz, CDCl₃) δ 8.50 (d, J=4.7 Hz, 1H), 7.91-7.81 (m, 2H), 7.51-7.41 (m, 2H), 7.14 (dd, J=8.1, 4.6 Hz, 1H), 7.10-7.01 (m, 2H), 6.71 (d, J=8.1 Hz, 1H), 5.30 (t, J=6.5 Hz, 1H), 5.16 (d, J=16.0 Hz, 1H), 4.59 (d, J=15.8 Hz, 1H), 3.58 (dd, J=15.1, 6.3 Hz, 1H), 3.30 (dd, J=15.1, 6.7 Hz, 1H).

### 6-((1-((3,6-dihydro-2H-pyran-4-yl)methyl)-3-oxoisoindolin-2-yl)methyl)benzo[d]oxazol-2(3H)-one (225)

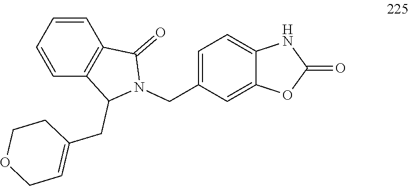 [SMILES: O=C1c2ccccc2C(CC2=CCOCC2)N1Cc1ccc2[nH]c(=O)oc2c1]

The title compound 225 was prepared according to general procedure E as a white solid (9 mg, 22.5%). MS (ESI) m/z 377 [M+H]⁺. ¹H NMR (400 MHz, CD₃OD) δ 7.82 (d, J=7.5 Hz, 1H), 7.64-7.57 (m, 1H), 7.57-7.48 (m, 2H), 7.19 (s, 1H), 7.13 (d, J=8.0 Hz, 1H), 7.05 (d, J=8.0 Hz, 1H), 5.43 (s, 1H), 5.27 (d, J=15.3 Hz, 1H), 4.63 (dd, J=6.8, 4.5 Hz, 1H), 4.47 (d, J=15.3 Hz, 1H), 4.05-3.96 (m, 2H), 3.61 (t, J=5.4 Hz, 2H), 2.79 (dd, J=15.3, 4.3 Hz, 1H), 2.57 (dd, J=14.4, 6.7 Hz, 1H), 1.86-1.70 (m, 2H).

### 6-((1-oxo-3-((tetrahydro-2H-pyran-4-yl)methyl)isoindolin-2-yl)methyl)benzo[d]oxazol-2(3H)-one (226)

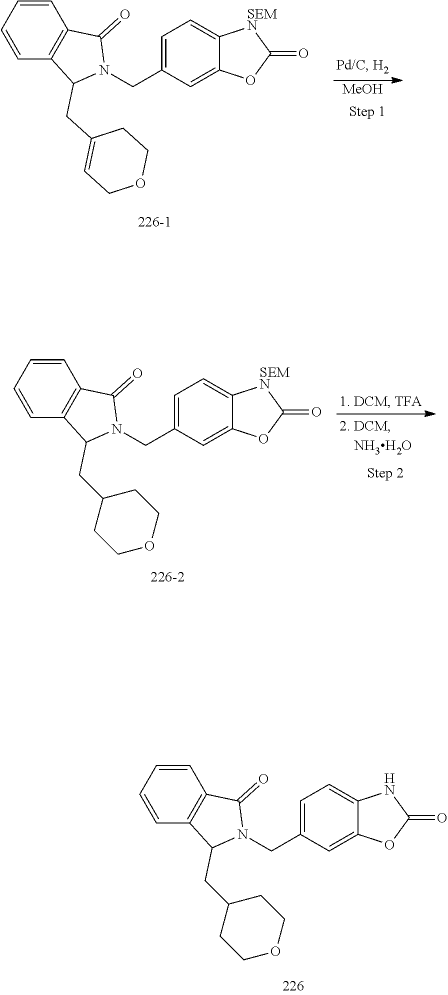 [SMILES: "MeOH" ; C[Si](C)(C)CCOCn1c(=O)oc2cc(CN3C(=O)c4ccccc4C3CC3=CCOCC3)ccc21 ; C[Si](C)(C)CCOCn1c(=O)oc2cc(CN3C(=O)c4ccccc4C3CC3CCOCC3)ccc21 ; O=C1c2ccccc2C(CC2CCOCC2)N1Cc1ccc2[nH]c(=O)oc2c1]

### Step 1. 6-((1-oxo-3-((tetrahydro-2H-pyran-4-yl)methyl)isoindolin-2-yl)methyl)-3-((2-(trimethylsilyl)ethoxy)methyl)benzo[d]oxazol-2(3H)-one (226-2)

In a stainless steel autoclave, 6-((1-((3,6-dihydro-2H-pyran-4-yl)methyl)-3-oxoisoindolin-2-yl)methyl)-3-((2-(trimethylsilyl)ethoxy)methyl)benzo[d]oxazol-2(3H)-one (100 mg, 0.20 mmol) was dissolved in methanol (10 mL) to which was added Pd/C (10 mg). The autoclave was charged to 20 bar with hydrogen and the reaction mixture was stirred for 2 h at r.t. The mixture was filtered through celite and the filtrates concentrated under reduced pressure to afford 6-((1-oxo-3-((tetrahydro-2H-pyran-4-yl)methyl)isoindolin-2-yl)methyl)-3-((2-(trimethylsilyl)ethoxy) methyl)benzo[d]oxazol-2(3H)-one (100 mg). The product was used in the next step without further purification.

### Step 2. 6-((1-oxo-3-((tetrahydro-2H-pyran-4-yl)methyl)isoindolin-2-yl)methyl)benzo[d]oxazol-2(3H)-one (226)

To a solution of 6-((1-oxo-3-((tetrahydro-2H-pyran-4-yl)methyl)isoindolin-2-yl)methyl)-3-((2-(trimethylsilyl)ethoxy)methyl)benzo[d]oxazol-2(3H)-one (100 mg, 0.09 mmol) in DCM (3 mL) was added TFA (1 mL). The mixture was stirred for 3 h and then concentrated under vacuum. The residue was taken in DCM (3 mL) and ammonium hydroxide (1 mL) was added at 0′C. This suspension was allowed to stir for an additional 1 h. The mixture was concentrated and purified by prep HPLC to give 6-((1-oxo-3-((tetrahydro-2H-pyran-4-yl)methyl)isoindolin-2-yl)methyl) benzo[d]oxazol-2(3H)-one as a white solid (6 mg, 8.1%). MS (ESI) m/z 379 [M+H]⁺. ¹H NMR (400 MHz, CD₃OD) δ 7.83 (d, J=7.6 Hz, 1H), 7.62 (t, J=7.4 Hz, 1H), 7.60-7.49 (m, 2H), 7.23 (s, 1H), 7.17 (d, J=8.0 Hz, 11H), 7.06 (d, J=8.0 Hz, 11H), 5.20 (d, J=15.2 Hz, 1H), 4.67-4.57 (m, 1H), 4.47 (d, J=15.2 Hz, 1H), 3.84-3.75 (m, 1i), 3.70 (dd, J=11.3, 3.6 Hz, 1H), 3.23-3.09 (m, 2H), 2.08-1.84 (m, 2H), 1.47-1.37 (m, 1H), 1.36-1.21 (m, 2H), 1.21-1.02 (m, 2H).

### 6-((1-((4,6-dichloropyridazin-3-yl)methyl)-3-oxoisoindolin-2-yl)methyl)benzo[d]oxazol-2(3H)-one (227)

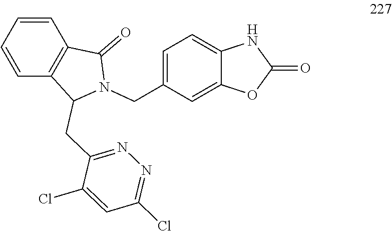 [SMILES: O=C1c2ccccc2C(Cc2nnc(Cl)cc2Cl)N1Cc1ccc2[nH]c(=O)oc2c1]

The title compound 227 was prepared according to general procedure E using 4,6-dichloro-3-methylpyridazine as a starting material to afford a white solid (2 mg, 5%). MS (ESI) m/z 441 [M+H]⁺. ¹H NMR (400 MHz, CD₃OD) δ 7.78 (s, 1H), 7.75-7.69 (m, 1H), 7.52-7.39 (m, 2H), 7.28 (d, J=7.4 Hz, 1H), 6.94 (s, 1H), 6.89-6.85 (m, 2H), 5.26 (t, J=5.8 Hz, 1H), 4.96 (d, J=15.6 Hz, 1H), 4.65 (d, J=15.6 Hz, 1H), 3.64 (dd, J=15.9, 5.8 Hz, 1H), 3.45 (dd, J=15.9, 5.8 Hz, 1H).

### 3-((2-((1H-benzo[d][1,2,3]triazol-5-yl)methyl)-3-oxoisoindolin-1-yl)methyl)-4-chloropicolinonitrile (228)

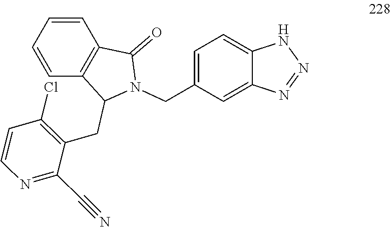 [SMILES: N#Cc1nccc(Cl)c1CC1c2ccccc2C(=O)N1Cc1ccc2[nH]nnc2c1]

The title compound 228 was prepared according to the procedure described for compound 212 as a white solid (50 mg, 42%). MS (ESI) m/z 415 [M+H]⁺. ¹H NMR (400 MHz, DMSO-d₆) δ 15.66 (s, 1H), 8.67 (d, J=5.3 Hz, 1H), 7.96 (d, J=5.3 Hz, 1H), 7.89 (d, J=8.6 Hz, 1H), 7.82 (d, J=7.5 Hz, 1H), 7.77 (s, 1H), 7.55 (t, J=7.4 Hz, 1H), 7.48 (td, J=7.5, 1.3 Hz, 1H), 7.31 (d, J=8.6 Hz, 1H), 6.58 (d, J=7.5 Hz, 1H), 5.35 (d, J=15.6 Hz, 1H), 4.81 (dd, J=9.4, 5.6 Hz, 1H), 4.65 (d, J=15.6 Hz, 1H), 3.88 (dd, J=14.0, 5.6 Hz, 1H), 2.90 (dd, J=14.0, 9.4 Hz, 1H).

### (S)-3-((2-((1H-benzo[d][1,2,3]triazol-5-yl)methyl)-3-oxoisoindolin-1-yl)methyl)-4-chloropicolinonitrile (229) and (R)-3-((2-((1H-benzo[d][1,2,3]triazol-5-yl)methyl)-3-oxoisoindolin-1-yl)methyl)-4-chloropicolinonitrile (230)

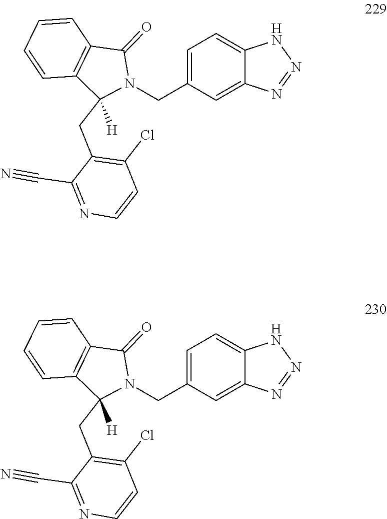 [SMILES: N#Cc1nccc(Cl)c1C[C@H]1c2ccccc2C(=O)N1Cc1ccc2[nH]nnc2c1 ; N#Cc1nccc(Cl)c1C[C@@H]1c2ccccc2C(=O)N1Cc1ccc2[nH]nnc2c1]

Chiral separation of 228 by chiral prep-HPLC (column: CHIRALPAK IG; column size: 2 cm×25 cm, 5 μm; mobile phase A: Hex:DCM (0.5% 2 mM NH₃-MeOH); mobile phase B: EtOH) was conducted to afford two enantiomers: compound 229 and compound 230. One of the two enantiomers was a white solid, with RT=7.184 min (CHIRAL HPLC, column: CHIRALPAK IG-3; column size: 4.6*50 mm, 3 μm; mobile phase: (Hex:DCM=3:1) (0.1% DEA):EtOH=90:10; flow: 1.0 mL/min). Mass (m/z): 415 [M+H]⁺. The other one of the two enantiomers was a white solid, RT=8.249 min (CHIRAL HPLC, column: CHIRALPAK IG-3; column size: 4.6*50 mm, 3 11 m; mobile phase: (Hex:DCM=3:1) (0.1% DEA):EtOH=90:10; flow: 1.0 mL/min). Mass (m/z): 415 [M+H]⁺.

### 6-((1-((5-methyloxazol-4-yl)methyl)-3-oxoisoindolin-2-yl)methyl)benzo[d]oxazol-2(3H)-one (231)

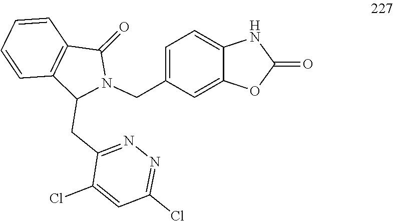 [SMILES: O=C1c2ccccc2C(Cc2nnc(Cl)cc2Cl)N1Cc1ccc2[nH]c(=O)oc2c1]

The title compound 231 was prepared according to general procedure E as a white solid (13 mg, 10.3%). MS (ESI) m/z 376 [M+H]⁺. ¹H NMR (400 MHz, DMSO-d₆) δ 11.62 (s, 1H), 8.05 (s, 1H), 7.63 (d, J=7.4 Hz, 1H), 7.54 (t, J=7.5 Hz, 1H), 7.49-7.38 (m, 2H), 7.21 (s, 1H), 7.04 (s, 2H), 5.13 (d, J=15.3 Hz, 1H), 4.68 (dd, J=6.3, 4.1 Hz, 1H), 4.46 (d, J=15.3 Hz, 1H), 3.20 (dd, J=14.9, 4.2 Hz, 1H), 3.02 (dd, J=14.9, 6.3 Hz, 1H), 1.93 (s, 3H).

### 6-((1-((4-chloro-1-methyl-1H-pyrazol-3-yl)methyl)-3-oxoisoindolin-2-yl)methyl)benzo[d]oxazol-2(3H)-one (232)

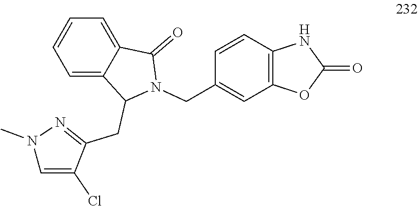 [SMILES: Cn1cc(Cl)c(CC2c3ccccc3C(=O)N2Cc2ccc3[nH]c(=O)oc3c2)n1]

The title compound 232 was prepared according to general procedure E as a white solid (23 mg, 15.4%). MS (ESI) m/z 409 [M+H]⁺. ¹H NMR (400 MHz, DMSO-d₆) δ 11.62 (s, 1H), 7.82 (s, 1H), 7.77-7.60 (m, 1H), 7.59-7.39 (m, 2H), 7.23 (d, J=7.5 Hz, 1H), 7.13 (d, J=1.4 Hz, 1H), 7.09-6.91 (m, 2H), 5.15 (d, J=15.2 Hz, 1H), 4.72 (dd, J=7.2, 5.0 Hz, 1H), 4.40 (d, J=15.2 Hz, 1H), 3.74 (s, 3H), 3.26 (dd, J=14.8, 5.0 Hz, 1H), 2.91 (dd, J=14.8, 7.2 Hz, 1H).

### 6-((1-((4-fluoropyridin-2-yl)methyl)-3-oxoisoindolin-2-yl)methyl)benzo[d]oxazol-2(3H)-one (233)

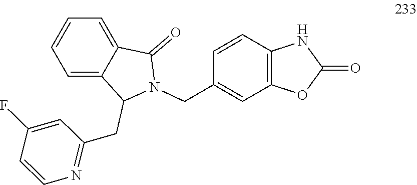 [SMILES: O=C1c2ccccc2C(Cc2cc(F)ccn2)N1Cc1ccc2[nH]c(=O)oc2c1]

The title compound 233 was prepared according to general procedure E as a white solid (21 mg, 23.5%). MS (ESI) m/z 390 [M+H]⁺. ¹H NMR (400 MHz, DMSO-d₆) δ 11.62 (s, 1H), 8.49 (dd, J=9.1, 5.7 Hz, 1H), 7.73-7.63 (m, 1H), 7.60-7.42 (m, 2H), 7.23-7.12 (m, 2H), 7.11 (d, J=1.4 Hz, 1H), 7.06-6.89 (m, 3H), 5.06 (d, J=15.3 Hz, 1H), 4.99 (t, J=5.8 Hz, 1H), 4.45 (d, J=15.4 Hz, 1H), 3.50 (dd, J=1 4.2, 5.1 Hz, 1H), 3.25 (dd, J=14.2, 6.7 Hz, 1H).

### 6-((1-((1-methyl-2-oxo-1,2-dihydropyridin-3-yl)methyl)-3-oxoisoindolin-2-yl)methyl) benzo[d]oxazol-2(3H)-one (234)

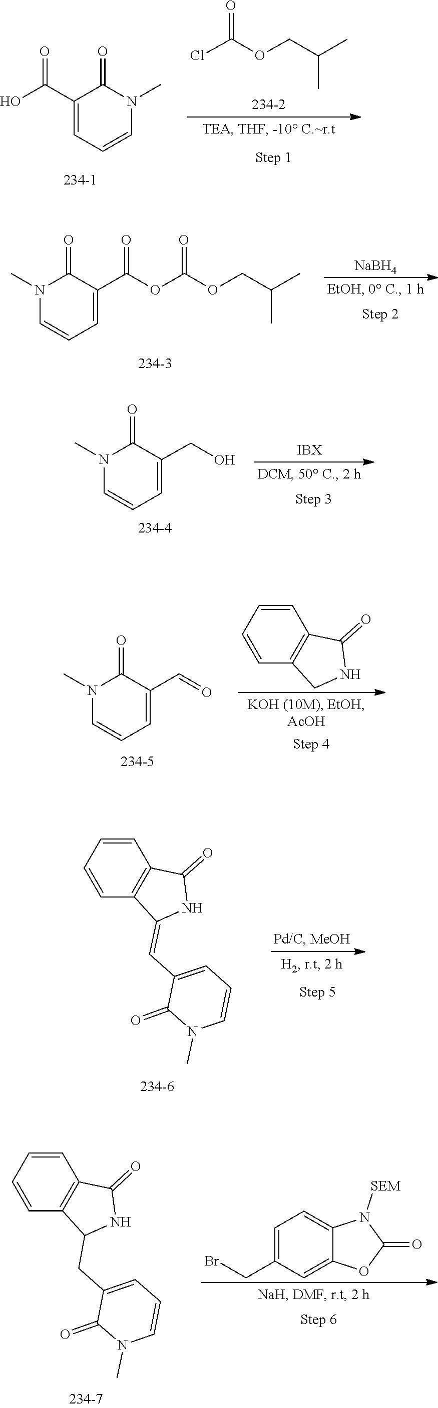

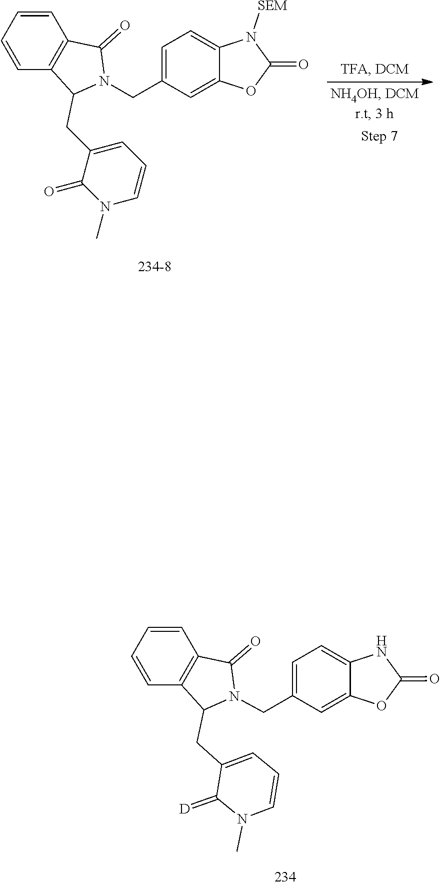

### Step 1. (isobutyl carbonic) 1-methyl-2-oxo-1,2-dihydropyridine-3-carboxylic anhydride (234-3)

Under an ice-salt bath, isobutyl carbonochloridate (1.34 g, 9.79 mmol) was added dropwise to a stirred solution of 1-methyl-2-oxo-1,2-dihydropyridine-3-carboxylic acid (1.0 g, 6.53 mmol) and TEA (1.0 g, 9.79 mmol) in THF (15 mL). The mixture was stirred at r.t. for 2 h. The reaction mixture was filtered off and the filtrate was concentrated. The residue was used directly for the next step.

### Step 2. 3-(hydroxymethyl)-1-methylpyridin-2(1H)-one (234-4)

Under an ice-water bath, NaBH₄ (740 mg, 19.5 mmol) was added in several portions to a stirred solution of (isobutyl carbonic) 1-methyl-2-oxo-1,2-dihydropyridine-3-carboxylic anhydride (1.65 g, 6.53 mmol) in EtOH (15 mL). The mixture was stirred at 0° C. for 1 h. The reaction mixture was quenched by 1M HCl (2 mL) and concentrated under vacuum. The residue was triturated with MeOH/DCM (15:1) and filtered off. The filtrate was concentrated to afford the title compound (780 mg, 86%) as a yellow solid. MS (ESI) m/z 140 [M+H]⁺.

### Step 3. 1-methyl-2-oxo-1,2-dihydropyridine-3-carbaldehyde (234-5)

To a stirred solution of 1-methyl-2-oxo-1,2-dihydropyridine-3-carboxylic acid (600 mg, 4.31 mmol) in DCM (25 mL) was added IBX (1.45 g, 5.17 mmol). The mixture was stirred at 50° C. for 2 h. The reaction mixture was filtered off and the filtrate was concentrated to afford the title compound (553 mg, 93.5%) as a light yellow solid. MS (ESI) m/z 138 [M+H]⁺.

### Step 4. (Z)-3-((1-methyl-2-oxo-1,2-dihydropyridin-3-yl)methylene)isoindolin-1-one (234-6)

To a stirred solution of isoindolin-1-one (537 mg, 4.03 mmol) and 1-methyl-2-oxo-1,2-dihydropyridine-3-carbaldehyde (553 mg, 4.03 mmol) in EtOH (25 mL) was added KOH (10M, 10 mL). The mixture was stirred at 80° C. overnight. The reaction mixture was cooled and adjusted to pH about 7-8 with AcOH. The mixture was extracted with DCM (3×50 mL). The combined organic phase was washed with brine, dried over anhydrous Na₂SO₄ and concentrated. The residue was purified by a silica gel column eluting with DCM and methanol (15:1) to afford the title compound (287 mg, 28.2%) as a yellow solid. MS (ESI) m/z 253.1 [M+H]⁺.

### Step 5. 3-((1-methyl-2-oxo-1,2-dihydropyridin-3-yl)methyl)isoindolin-1-one (234-7)

To a stirred solution of (Z)-3-((1-methyl-2-oxo-1,2-dihydropyridin-3-yl)methylene)isoindolin-1-one (160 mg, 0.63 mmol) in MeOH (10 mL) was added Pd/C (16 mg, 0.06 mmol). The mixture was evacuated and backfilled three times with hydrogen. The resulting mixture was stirred at r.t. for 2 h. The reaction mixture was filtered off and the filtrate was concentrated to afford the title compound (84 mg, 52.1%) as a light yellow solid. MS (ESI) m/z 255 [M+H]⁺.

### Step 6. 6-((1-((1-methyl-2-oxo-1,2-dihydropyridin-3-yl)methyl)-3-oxoisoindolin-2-yl)methyl)-3-((2-(trimethylsilyl)ethoxy)methyl)benzo[d]oxazol-2(3H)-one (234-8)

Under ice-water bath cooling, NaH (60%, 14.5 mg, 0.36 mmol) was added portion-wise to a stirred mixture of 3-((1-methyl-2-oxo-1,2-dihydropyridin-3-yl)methyl)isoindolin-1-one (84 mg, 0.33 mmol) in DMF (15 mL). The mixture was stirred at 0° C. for 30 min. 6-(bromomethyl)-3-((2-(trimethylsilyl)ethoxy) methyl)benzo[d]oxazol-2(3H)-one (130 mg, 0.36 mmol) was added in portions. The resulting mixture was stirred at r.t. for 3 h. The mixture was quenched by ice-water and extracted with EtOAc (3×10 mL). The combined organic phase was washed with brine (2×50 mL), dried over anhydrous Na₂SO₄ and concentrated to afford the title compound (130 mg, crude) as a light yellow solid. MS (ESI) m/z 532 [M+H]⁺.

### Step 7. 6-((1-((1-methyl-2-oxo-1,2-dihydropyridin-3-yl)methyl)-3-oxoisoindolin-2-yl) methyl) benzo[d]oxazol-2(3H)-one (234)

To a solution of 6-((1-((1-methyl-2-oxo-1,2-dihydropyridin-3-yl)methyl)-3-oxoisoindolin-2-yl)methyl)-3-((2-(trimethylsilyl)ethoxy)methyl)benzo[d]oxazol-2(3H)-one (130 mg, 0.24 mmol) in DCM (5 mL) was added trifluoroacetic acid (1 mL). The mixture was stirred at r.t. for 2h, and then concentrated under vacuum. The residue was dissolved with DCM (2 mL), and NH₄OH (0.5 mL) was added dropwise. The mixture was stirred at r.t. for 1 h. The reaction mixture was concentrated, and the residue was purified by Prep-HPLC to afford title compound as a white solid (20 mg, 20%). MS (ESI) m/z 402 [M+H]⁺. ¹H NMR (400 MHz, DMSO-d₆) δ 11.60 (s, 1H), 7.69 (d, J=7.4 Hz, 1H), 7.64 (dd, J=6.8, 2.0 Hz, 1H), 7.58-7.43 (m, 2H), 7.26 (d, J=7.4 Hz, 1H), 7.09 (d, J=6.4 Hz, 2H), 7.03-6.94 (m, 2H), 6.10 (t, J=6.8 Hz, 1H), 5.08 (d, J=15.2 Hz, 1H), 4.84 (t, J=5.9 Hz, 1H), 4.43 (d, J=15.2 Hz, 1H), 3.41 (s, 3H), 3.19 (dd, J=13.6, 5.2 Hz, 1H), 2.82 (dd, J=13.6, 6.8 Hz, 1H).

### Mixture of 6-(((R)-1-(((R)-1-methyl-2-oxopiperidin-3-yl)methyl)-3-oxoisoindolin-2-yl) methyl)benzo[d]oxazol-2(3H)-one (235) and 6-(((S)-1-(((S)-1-methyl-2-oxopiperidin-3-yl)methyl)-3-oxoisoindolin-2-yl)methyl)benzo[d]oxazol-2(3H)-one (236), and mixture of 6-(((R)-1-(((S)-1-methyl-2-oxopiperidin-3-yl)methyl)-3-oxoisoindolin-2-yl)methyl)benzo[d]oxazol-2(3H)-one (237) and 6-(((S)-1-(((R)-1-methyl-2-oxopiperidin-3-yl)methyl)-3-oxoisoindolin-2-yl)methyl)benzo[d]oxazol-2(3H)-one (238)

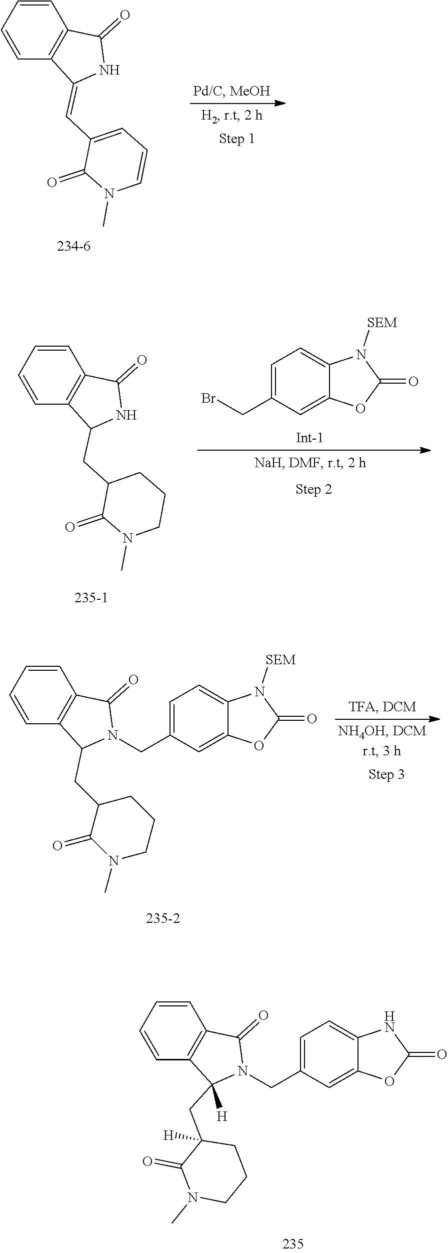 [SMILES: Cn1cccc(/C=C2\NC(=O)c3ccccc32)c1=O ; CN1CCCC(CC2NC(=O)c3ccccc32)C1=O ; C[Si](C)(C)CCOCn1c(=O)oc2cc(CBr)ccc21 ; CN1CCCC(CC2c3ccccc3C(=O)N2Cc2ccc3c(c2)oc(=O)n3COCC[Si](C)(C)C)C1=O ; CN1CCC[C@H](C[C@@H]2c3ccccc3C(=O)N2Cc2ccc3[nH]c(=O)oc3c2)C1=O]

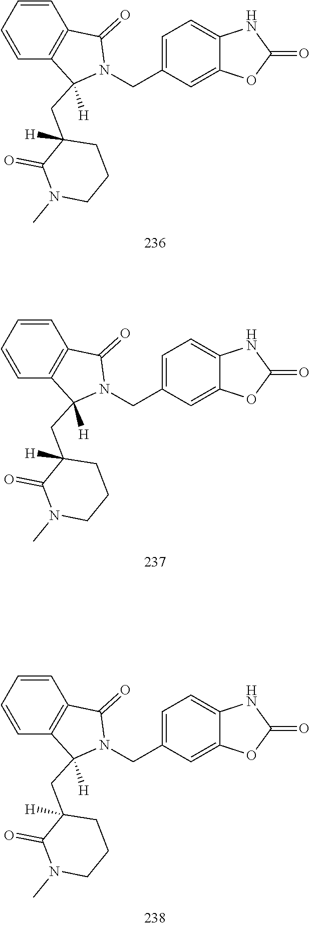 [SMILES: CN1CCC[C@@H](C[C@H]2c3ccccc3C(=O)N2Cc2ccc3[nH]c(=O)oc3c2)C1=O ; CN1CCC[C@@H](C[C@@H]2c3ccccc3C(=O)N2Cc2ccc3[nH]c(=O)oc3c2)C1=O ; CN1CCC[C@H](C[C@H]2c3ccccc3C(=O)N2Cc2ccc3[nH]c(=O)oc3c2)C1=O]

### Step 1. 3-((1-methyl-2-oxopiperidin-3-yl)methyl)isoindolin-1-one (235-1)

To a stirred solution of (Z)-3-((1-methyl-2-oxo-1,2-dihydropyridin-3-yl)methylene)isoindolin-1-one (127 mg, 0.63 mmol) in MeOH (10 mL) was added Pd/C (16 mg, 0.06 mmol). The mixture was evacuated and backfilled three times with hydrogen. The resulting mixture was stirred at r.t. overnight. The reaction mixture was filtered off and the filtrate was concentrated to afford the title compound (116 mg, 89.2%) as a light yellow solid. MS (ESI) m/z 259 [M+H]⁺.

### Step 2. 6-((1-((1-methyl-2-oxopiperidin-3-yl)methyl)-3-oxoisoindolin-2-yl)methyl)-3-((2-(trimethylsilyl)ethoxy)methyl)benzo[d]oxazol-2(3H)-one (235-2)

Under ice-water bath cooling, NaH (60%, 20 mg, 0.49 mmol) was added portion-wise to a stirred mixture of 3-((1-methyl-2-oxopiperidin-3-yl)methyl)isoindolin-1-one (116 mg, 0.45 mmol) in DMF (15 mL). The mixture was stirred at 0° C. for 30 min. 6-(bromomethyl)-3-((2-(trimethylsilyl) ethoxy) methyl) benzo[d]oxazol-2(3H)-one (177 mg, 0.49 mmol) was added in portions. The resulting mixture was stirred at r.t. for 3 h. The mixture was quenched by ice-water and extracted with EtOAc (3×10 mL). The combined organic phase was washed with brine (2×50 mL), dried over anhydrous Na₂SO₄ and concentrated to afford the title compound (145 mg, crude) as a light yellow solid.

### Step 3. Mixture of 6-(((R)-1-(((R)-1-methyl-2-oxopiperidin-3-yl)methyl)-3-oxoisoindolin-2-yl)methyl)benzo[d]oxazol-2(3H)-one (235) and 6-(((S)-1-(((S)-1-methyl-2-oxopiperidin-3-yl)methyl)-3-oxoisoindolin-2-yl)methyl)benzo[d]oxazol-2(3H)-one (236) and mixture of 6-(((R)-1-(((S)-1-methyl-2-oxopiperidin-3-yl) methyl)-3-oxoisoindolin-2-yl)methyl)benzo[d]oxazol-2(3H)-one (237) and 6-(((S)-1-(((R)-1-methyl-2-oxopiperidin-3-yl)methyl)-3-oxoisoindolin-2-yl)methyl)benzo[d]oxazol-2(3H)-one (238)

To a solution of 6-((1-((1-methyl-2-oxopiperidin-3-yl)methyl)-3-oxoisoindolin-2-yl)methyl)-3-((2-(trimethylsilyl)ethoxy)methyl)benzo[d]oxazol-2(3H)-one (145 mg, 0.27 mmol) in DCM (5 mL) was added trifluoroacetic acid (1 mL). The mixture was stirred at r.t. for 2h, and then concentrated under vacuum. The residue was dissolved with DCM (2 mL), and NH₄OH (0.5 mL) was added dropwise. The mixture was stirred at r.t. for 1 h. The reaction mixture was concentrated, and the residue was purified by Prep-HPLC to afford two diastereomers (each diastereomers contains two enantiomers): mixture of 235 and 236, and mixture of 237 and 238. One of the two diastereomers was a white solid (5 mg, 4.5%), with RT=3.63 min (HPLC), MS (ESI) m/z 406 [M+H]⁺. ¹H NMR (400 MHz, DMSO-d₆) δ 11.62 (s, 1H), 7.72 (d, J=7.5 Hz, 1H), 7.65-7.56 (m, 2H), 7.53-7.47 (m, 1H), 7.22 (d, J=1.4 Hz, 1H), 7.08-6.97 (m, 2H), 5.11 (d, J=15.1 Hz, 1H), 4.73 (dd, J=6.3, 4.4 Hz, 1H), 4.33 (d, J=15.2 Hz, 1H), 3.26-3.09 (m, 2H), 2.77 (s, 3H), 2.71-2.61 (m, 1H), 2.05 (dd, J=9.4, 6.3 Hz, 1H), 1.80-1.40 (m, 5H). The other one of the two diastereomers was a white solid (10 mg, 9%), with RT=3.54 min (HPLC), MS (ESI) m/z 406 [M+H]+, ¹H NMR (400 MHz, DMSO-d₆) δ 11.63 (s, 1H), 7.74 (d, J=7.4 Hz, 11H), 7.60 (d, J=4.0 Hz, 2H), 7.55-7.47 (m, 1H), 7.18 (d, J=1.3 Hz, 1H), 7.11-7.01 (m, 2H), 5.19-5.09 (m, 1H), 4.53 (dd, J=7.1, 2.4 Hz, 1H), 4.43 (d, J=15.2 Hz, 1H), 3.24-3.11 (m, 2H), 2.79 (s, 3H), 2.50-2.41 (m, 1H), 1.99 (td, J=9.1, 3.1 Hz, 2H), 1.69-1.59 (m, 1H), 1.55-1.43 (m, 1H), 1.32 (d, J=12.6 Hz, 1H), 1.20 (dd, J=15.2, 6.9 Hz, 1H).

### 2-([1,2,4]triazolo[4,3-a]pyridin-7-ylmethyl)-3-((3-bromopyridin-2-yl)methyl)isoindolin-1-one (239)

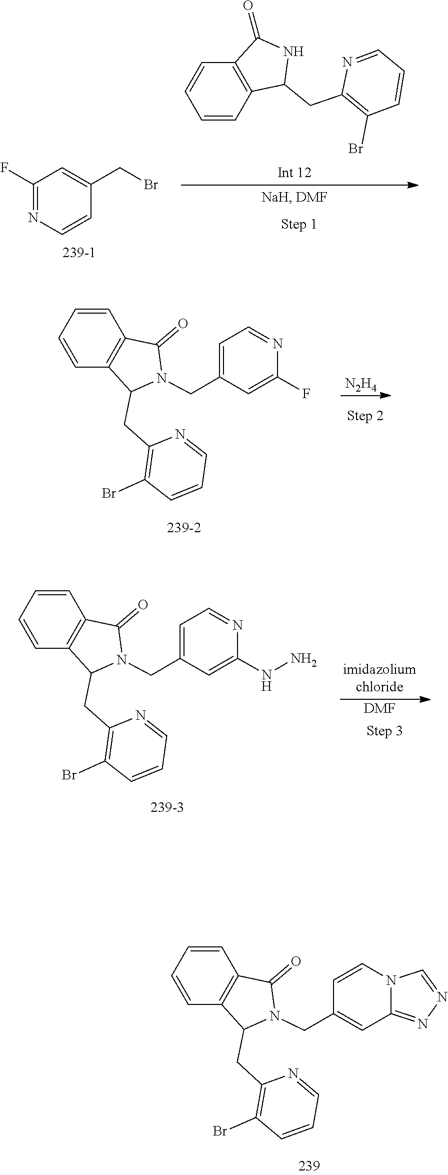

### Step 1. 3-((3-bromopyridin-2-yl)methyl)-2-((2-fluoropyridin-4-yl)methyl) isoindolin-1-one (239-2)

To a solution of NaH (28 mg, 0.70 mmol) in DMF (1 mL), was added 3-[(3-bromo-2-pyridyl)methyl]isoindolin-1-one (177 mg, 0.59 mmol) in DMF (1 mL) at 0° C. under N₂. After addition, the mixture was stirred at 0° C. for 0.5 h. Then added 4-(bromomethyl)-2-fluoro-pyridine (112 mg, 0.59 mmol) in DMF (1 mL). After addition, the mixture was stirred at 0° C. for 0.5 h. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with flash (PE/EtOAc=1:1) to give 3-((3-bromopyridin-2-yl)methyl)-2-((2-fluoropyridin-4-yl)methyl) isoindolin-1-one (100 mg, 41.6%) as a yellow oil. MS (ESI) m/z 412 [M+H]⁺.

### Step 2. 3-[(3-bromo-2-pyridyl)methyl]-2-[(2-hydrazino-4-pyridyl)methyl]isoindolin-1-one (239-3)

3-[(3-bromo-2-pyridyl)methyl]-2-[(2-fluoro-4-pyridyl)methyl]isoindolin-1-one (100 mg, 0.24 mmol) was dissolved in Hydrazinium hydroxide solution (1.0 mL) under N₂. After addition, the mixture was stirred at 110° C. for 0.5 h. LC-MS showed the starting material was mostly converted to the product. The reaction mixture was concentrated to give the product 3-[(3-bromo-2-pyridyl)methyl]-2-[(2-hydrazino-4-pyridyl)methyl]isoindolin-1-one (100 mg, 77.7%) as a yellow solid. MS (ESI) m/z 424 [M+H]⁺.

### Step 3. 2-((3H-414-[1,2,4]triazolo[4,3-a]pyridin-7-yl)methyl)-3-((3-bromopyridin-2-yl) methyl)isoindolin-1-one (239)

To a solution of 3-[(3-bromo-2-pyridyl)methyl]-2-[(2-hydrazino-4-pyridyl)methyl]isoindolin-1-one (50 mg, 0.12 mmol) in DMF (1 mL) was added imidazolium chloride (2 mg, 0.012 mmol) under N₂. After addition, the mixture was stirred at 160° C. for 0.5 h with microwave. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with C18 flash to give the product 2-((3H-414-[1,2,4]triazolo[4,3-a]pyridin-7-yl)methyl)-3-((3-bromopyridin-2-yl)methyl) isoindolin-1-one (10 mg, 17.5%) as a white solid. MS (ESI) m/z 434 [M+H]⁺. ¹H NMR (400 MHz, DMSO-d₆) δ 9.19 (d, J=0.9 Hz, 1H), 8.52 (dd, J=4.6, 1.5 Hz, 1H), 8.45 (dd, J=7.1, 1.1 Hz, 1H), 7.97 (dd, J=8.0, 1.5 Hz, 1H), 7.78-7.74 (m, 1H), 7.56-7.49 (m, 2H), 7.36 (s, 1H), 7.26 (dd, J=8.1, 4.6 Hz, 1H), 7.14-7.11 (m, 1H), 6.72 (dd, J=7.1, 1.6 Hz, 1H), 5.28 (t, J=6.4 Hz, 1H), 4.98 (d, J=16.0 Hz, 1H), 4.60 (d, J=16.0 Hz, 1H), 3.58 (dd, J=15.3, 6.1 Hz, 1H), 3.27 (dd, J=15.3, 6.8 Hz, 1H).

### 2-((1H-indazol-5-yl)methyl)-3-((3-bromopyridin-2-yl)methyl)isoindolin-1-one (240)

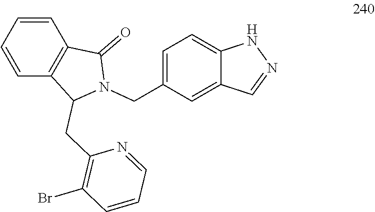 [SMILES: O=C1c2ccccc2C(Cc2ncccc2Br)N1Cc1ccc2[nH]ncc2c1]

The title compound 240 was prepared according to general procedure F as a white solid (8 mg, 32.8%). MS (ESI) m/z 433 [M+H]⁺. ¹H NMR (400 MHz, CDCl₃) δ 8.51 (s, 1H), 8.01 (s, 1H), 7.92 (d, J=7.6 Hz, 1H), 7.85 (d, J=7.9 Hz, 1H), 7.50-7.36 (m, 4H), 7.30-7.26 (m, 1H), 7.16 (br s, 1H), 6.99 (d, J=7.1 Hz, 1H), 5.35 (d, J=14.2 Hz, 1H), 5.23 (t, J=6.0 Hz, 1H), 4.50 (d, J=14.2 Hz, 1H), 3.60 (dd, J=14.2, 6.0 Hz, 1H), 3.25 (dd, J=14.2, 5.2 Hz, 1H).

### 5-((1-((3-bromopyridin-2-yl)methyl)-3-oxoisoindolin-2-yl)methyl)oxazolo[5,4-b]pyridin-2(1H)-one (241)

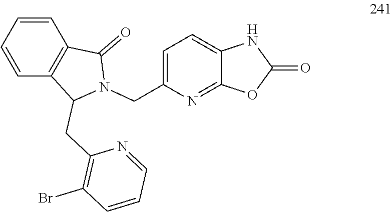 [SMILES: O=C1c2ccccc2C(Cc2ncccc2Br)N1Cc1ccc2[nH]c(=O)oc2n1]

The title compound 241 was prepared according to the procedure described for compound 223 using 3-amino-6-methylpyridin-2-ol as a starting material to afford a white solid (40 mg, 48.2%). MS (ESI) m/z 451 [M+H]⁺. ¹H NMR (400 MHz, CDCl₃) δ 8.62 (s, 1H), 7.99-7.85 (m, 3H), 7.57-7.51 (m, 1H), 7.51-7.45 (m, 2H), 7.25-7.20 (m, 1H), 7.15 (d, J=7.9 Hz, 1H), 7.04 (d, J=7.9 Hz, 1H), 5.45 (t, J=6.5 Hz, 1H), 5.20 (d, J=14.8 Hz, 1H), 4.49 (d, J=15.9 Hz, 1H), 3.64 (dd, J=15.1, 6.9 Hz, 1H), 3.45 (m, 1H).

### rel-(S)-6-((5-(cyclopropylamino)-3-methyl-1-oxoisoindolin-2-yl)methyl)benzo[d]oxazol-2(3H)-one (242)

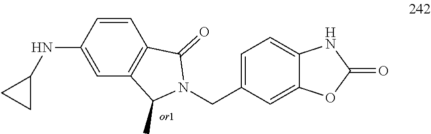 [SMILES: C[C@H]1c2cc(NC3CC3)ccc2C(=O)N1Cc1ccc2[nH]c(=O)oc2c1]

The title compound 242 was prepared according to the procedure described for compound 81 using the stereoisomer of Int 10 or Int 11 that has RT of 1.969 min as a starting material to afford a white solid (3 mg, 20%). MS (ESI) m/z 350 [M+H]⁺. ¹H NMR (400 MHz, CD₃OD) δ 7.42 (d, J=8.2 Hz, 1H), 7.09-7.01 (m, 2H), 6.94 (d, J=8.0 Hz, 1H), 6.72-6.68 (m, 2H), 4.98 (d, J=15.4 Hz, 1H), 4.33 (d, J=15.4 Hz, 1H), 4.25 (q, J=6.7 Hz, 1H), 2.35-2.30 (m, 1H), 1.31 (d, J=6.7 Hz, 3H), 0.69-0.64 (m, 2H), 0.43-0.28 (m, 2H).

### rel-(S)-6-((5-methoxy-3-methyl-1-oxoisoindolin-2-yl)methyl)benzo[d]oxazol-2(3H)-one (243)

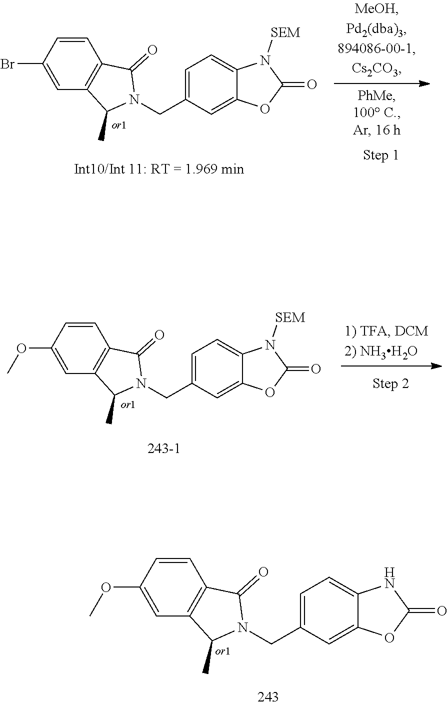 [SMILES: C[C@H]1c2cc(Br)ccc2C(=O)N1Cc1ccc2c(c1)oc(=O)n2COCC[Si](C)(C)C ; COc1ccc2c(c1)[C@H](C)N(Cc1ccc3c(c1)oc(=O)n3COCC[Si](C)(C)C)C2=O ; COc1ccc2c(c1)[C@H](C)N(Cc1ccc3[nH]c(=O)oc3c1)C2=O]

### Step 1. rel-(S)-6-((5-methoxy-3-methyl-1-oxoisoindolin-2-yl)methyl)-3-((2-(trimethylsilyl) ethoxy)methyl)benzo[d]oxazol-2(3H)-one (243-1)

To a solution of the stereoisomer of Int 10 or Int 11 that has RT of 1.969 min (139 mg, 0.27 mmol) and Cs₂CO₃ (224 mg, 0.69 mmol) in toluene (3 mL) were added MeOH (1 ml), tBu-Bippyphos (42 mg, 0.08 mmol) and Pd₂(dba)₃ (51 mg, 0.05 mmol) at r.t. The mixture was stirred for 16 h at 100° C. under Ar. The mixture was diluted with EtOAc and washed with water. The organic layer was separated, dried over anhydrous Na₂SO₄ and concentrated under vacuum. The residue was purified by silica gel flash column chromatography (CH₂Cl₂/EtOAc=2/1) to afford rel-(S)-6-((5-methoxy-3-methyl-1-oxoisoindolin-2-yl)methyl)-3-((2-(trimethylsilyl)ethoxy) methyl)benzo[d]oxazol-2(3H)-one (90 mg, 74%)) as a yellow solid. MS (ESI) m/z 455 [M+H]⁺.

### Step 2. rel-(S)-6-((5-methoxy-3-methyl-1-oxoisoindolin-2-yl)methyl)benzo[d]oxazol-2(3H)-one (243)

To a solution of 243-1 (90 mg 0.2 mmol) in DCM (2 mL) was added TFA (2 ml) at r.t. The mixture was stirred for 1 h at r.t. The mixture was concentrated under vacuum and added NH₄OH (2 ml). After reacted at r.t. for 1 h, the mixture was concentrated under vacuum. The residue was purified by silica gel flash column chromatography (CH₂Cl₂/MeOH=20/1) to afford rel-(S)-6-((5-methoxy-3-methyl-1-oxoisoindolin-2-yl)methyl)benzo[d]oxazol-2(3H)-one (17 mg, 23%) as a yellow solid. MS (ESI) m/z 325 [M+H]-. ¹H NMR (400 MHz, DMSO-d₆) δ 11.60 (s, 1H), 7.62 (d, J=8.4 Hz, 1H), 7.29-6.96 (m, 5H), 4.97 (d, J=15.2 Hz, 1H), 4.44-4.20 (m, 2H), 3.83 (s, 3H), 1.38 (d, J=6.7 Hz, 3H).

### 6-((1-methyl-3-oxo-4-(pyridin-3-yl)isoindolin-2-yl)methyl)benzo[d]oxazol-2(3H)-one (244)

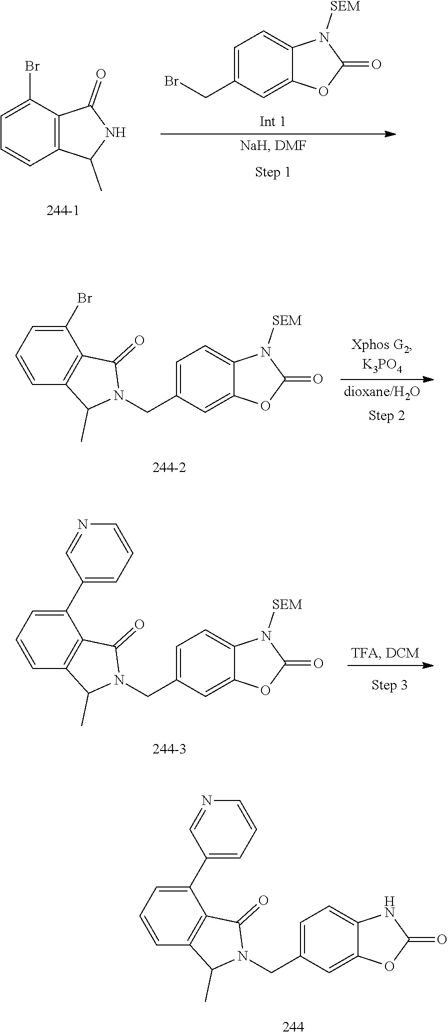 [SMILES: CC1NC(=O)c2c(Br)cccc21 ; C[Si](C)(C)CCOCn1c(=O)oc2cc(CBr)ccc21 ; CC1c2cccc(Br)c2C(=O)N1Cc1ccc2c(c1)oc(=O)n2COCC[Si](C)(C)C ; CC1c2cccc(-c3cccnc3)c2C(=O)N1Cc1ccc2c(c1)oc(=O)n2COCC[Si](C)(C)C ; CC1c2cccc(-c3cccnc3)c2C(=O)N1Cc1ccc2[nH]c(=O)oc2c1]

### Step 1. 6-((4-bromo-1-methyl-3-oxoisoindolin-2-yl)methyl)-3-((2-(trimethylsilyl)ethoxy) methyl)benzo[d]oxazol-2(3H)-one (244-2)

To a solution of 7-bromo-3-methylisoindolin-1-one (750 mg, 3.3 mmol) in DMF (30 mL) was added NaH (140 mg, 60% in mineral oil, 3.5 mmol) at 0° C. The reaction was stirred for 30 min at 0° C. Then 6-(bromomethyl)-3-((2-(trimethylsilyl)ethoxy) methyl)benzo[d]oxazol-2(3H)-one (1.2 g, 3.3 mmol) was added at 0° C. The reaction was stirred for 1 h at r.t. H₂O (50 mL) was added, and the mixture was extracted with EA (50 mL*2). The combined organic layers were washed with H₂O (100 mL*2) and brine (50 mL), dried over Na₂SO₄, filtered and concentrated. Then the residue was purified by flash column chromatography (PE/EA=5/l) to give 6-((4-bromo-1-methyl-3-oxoisoindolin-2-yl)methyl)-3-((2-(trimethylsilyl)ethoxy)methyl)benzo[d]oxazol-2(3H)-one (500 mg, 30%) as a white solid. MS (ESI) m/z 503 [M+H]⁺.

### Step 2. 6-((1-methyl-3-oxo-4-(pyridin-3-yl)isoindolin-2-yl)methyl)-3-((2-(trimethylsilyl) ethoxy)methyl)benzo[d]oxazol-2(3H)-one (244-3)

A mixture of 6-((4-bromo-1-methyl-3-oxoisoindolin-2-yl)methyl)-3-((2-(trimethylsilyl) ethoxy)methyl)benzo[d]oxazol-2(3H)-one (60 mg, 0.12 mmol), 3-(4,4,5,5-tetramethyl-1,3,2-dioxaborolan-2-yl)pyridine (25 mg, 0.12 mmol), Pd-xphos-G2 (19 mg, 0.24 mmol) and K₃PO₄ (51 mg, 0.24 mmol) in dioxane/H₂O (5 mL/1 mL) was stirred at 100° C. overnight. The mixture was filtered and extracted with EA (50 mL*2). The combined organic layers were washed with brine, dried over Na₂SO₄, filtered and concentrated. The residue was purified by trituration with PE to give 6-((1-methyl-3-oxo-4-(pyridin-3-yl)isoindolin-2-yl)methyl)-3-((2-(trimethylsilyl) ethoxy)methyl)benzo[d]oxazol-2(3H)-one (45 mg, 75%) as a yellow solid. MS (ESI) m/z 502 [M+H]⁺.

### Step 3. 6-((1-methyl-3-oxo-4-(pyridin-3-yl)isoindolin-2-yl)methyl)benzo[d]oxazol-2(3H)-one (244)

To a solution of 6-((1-methyl-3-oxo-4-(pyridin-3-yl)isoindolin-2-yl)methyl)-3-((2-(trimethylsilyl)ethoxy)methyl)benzo[d]oxazol-2(3H)-one (45 mg, 0.09 mmol) in DCM (2 mL) was added TFA (3 mL). The mixture was stirred at r.t. for 2 h. Then pH of the mixture was adjusted to 8 using Na₂CO₃ (aq.) and the mixture was extracted with DCM (10 mL*2). The combined organic layers were washed with Na₂CO₃ (aq.) (50 mL) and brine (50 mL), dried over Na₂SO₄, filtered and concentrated in vacuo and purified by prep-HPLC to give 6-((1-methyl-3-oxo-4-(pyridin-3-yl)isoindolin-2-yl)methyl)benzo[d]oxazol-2(3H)-one (16 mg, 48%) as a white solid. MS (ESI) m/z 372 [M+H]⁺. ¹H NMR (400 MHz, CD₃OD) δ 8.70 (d, J=1.6 Hz, 1H), 8.55 (dd, J=6.4, 4.8 Hz, 1H), 8.05-8.02 (m, 1H), 8.70 (t, J=7.6 Hz, 1H), 7.59 (d, J=7.6 Hz, 1H), 7.53-7.50 (m, 1H), 7.43 (d, J=7.6 Hz, 1H), 7.20 (s, 1H), 7.13 (d, J=8.0 Hz, 1H), 7.02 (d, J=8.4 Hz, 1H), 5.07 (d, J=15.2 Hz, 1H), 4.54-4.52 (m, 1H), 4.44 (d, J=15.6 Hz, 1H), 1.49 (d, J=6.8 Hz, 3H).

### 6-((4-cyclopropyl-3-methyl-1-oxoisoindolin-2-yl)methyl)benzo[d]oxazol-2(3H)-one (245)

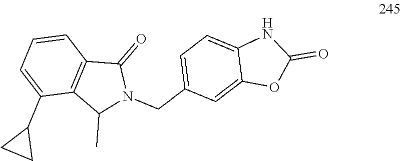 [SMILES: CC1c2c(cccc2C2CC2)C(=O)N1Cc1ccc2[nH]c(=O)oc2c1]

The title compound 245 was prepared according to the procedure described for compound 125 as a white solid (10 mg, 27.5%). MS (ESI) m/z 335 [M+H]⁺. ¹H NMR (400 MHz, DMSO-d₆) δ 11.22 (s, 1H), 7.50 (d, J=7.2 Hz, 1H), 7.39 (t, J=7.6 Hz, 1H), 7.2 (s, 1H), 7.12-7.08 (m, 2H), 7.03 (d, J=8.0 Hz, 1H), 5.08 (d, J=15.2 Hz, 1H), 4.62 (q, J=6.4 Hz, 1H), 4.37 (d, J=15.6 Hz, 1H), 2.00-1.96 (m, 1H), 1.49 (d, J=6.4 Hz, 3H), 1.05-0.99 (m, 1H), 0.93-0.85 (m, 2H), 0.63-0.59 (m, 1H).

### 6-((3,4-dimethyl-1-oxoisoindolin-2-yl)methyl)benzo[d]oxazol-2(3H)-one (246)

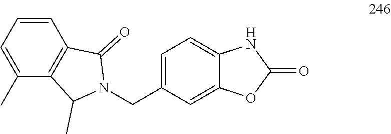 [SMILES: Cc1cccc2c1C(C)N(Cc1ccc3[nH]c(=O)oc3c1)C2=O]

The title compound 246 was prepared according to the procedure described for compound 163 as a white solid (24 mg, 52%). MS (ESI) m/z 309 [M+H]⁺. ¹H NMR (400 MHz, CD₃OD) δ 7.65-7.63 (m, 1H), 7.43-7.38 (m, 2H), 7.21 (s, 1H), 7.14 (d, J=8.0 Hz, 1H), 7.03 (d, J=8.4 Hz, 1H), 5.18 (d, J=15.2 Hz, 1H), 4.55-4.52 (m, 1H), 4.46 (d, J=15.2 Hz, 1H), 2.38 (s, 3H), 1.46 (d, J=6.4 Hz 3H).

### 6-((4-bromo-3-methyl-1-oxoisoindolin-2-yl)methyl)benzo[d]oxazol-2(3H)-one (247)

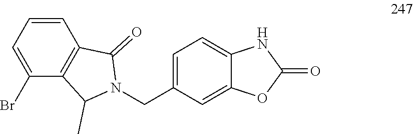 [SMILES: CC1c2c(Br)cccc2C(=O)N1Cc1ccc2[nH]c(=O)oc2c1]

The title compound 247 was prepared according to the procedure described for compound 48 as a white solid (40 mg, 54%). MS (ESI) m/z 373 [M+H]⁺. ¹H NMR (400 MHz, CD₃OD) δ 7.81 (d, J=7.2 Hz, 1H), 7.77 (d, J=7.6 Hz, 1H), 7.45 (t, J=8.0 Hz, 1H), 7.23 (s, 1H), 7.16 (dd, J=8.4, 0.8 Hz, 1H), 7.06 (d, J=8.0 Hz, 1H), 5.19 (d, J=15.2 Hz, 1H), 4.51-4.45 (m, 2H), 1.59 (d, J=6.8 Hz, 3H).

### 6-((1-((4-methylisoxazol-3-yl)methyl)-3-oxoisoindolin-2-yl)methyl)benzo[d]oxazol-2(3H)-one (248)

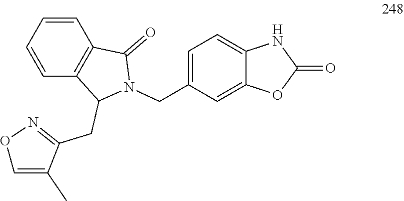 [SMILES: Cc1conc1CC1c2ccccc2C(=O)N1Cc1ccc2[nH]c(=O)oc2c1]

The title compound 248 was prepared according to the procedure described for compound 217 using ethyl 4-methylisoxazole-3-carboxylate as a starting material to afford a white solid (12 mg, 15%). MS (ESI) m/z 376 [M+H]⁺. ¹H NMR (400 MHz, DMSO-d₆) δ 8.48 (s, 1H), 8.39 (s, 1H), 7.66 (d, J=7.4 Hz, 1H), 7.59-7.50 (m, 1H), 7.50-7.45 (m, 1H), 7.42 (d, J=7.5 Hz, 1H), 7.16 (s, 1H), 7.06-6.96 (m, 2H), 5.15 (d, J=15.3 Hz, 1H), 4.78 (dd, J=6.7, 4.1 Hz, 1H), 4.41 (d, J=15.2 Hz, 1H), 3.44-3.39 (m, 1H), 3.17 (dd, J=15.3, 6.8 Hz, 1H), 1.68 (s, 3H).

### 6-((1-((5-chloro-1-methyl-1H-imidazol-4-yl)methyl)-3-oxoisoindolin-2-yl)methyl)benzo[d]oxazol-2(3H)-one (249)

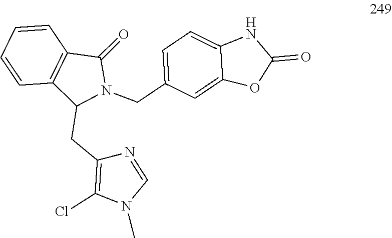 [SMILES: Cn1cnc(CC2c3ccccc3C(=O)N2Cc2ccc3[nH]c(=O)oc3c2)c1Cl]

The title compound 249 was prepared according to the procedure described for compound 217 using ethyl 5-chloro-1-methyl-1H-imidazole-4-carboxylate as starting material to afford a white solid (6 mg, 25%). MS (ESI) m/z 409 [M+H]⁺. ¹H NMR (400 MHz, CD₃OD) δ 7.88-7.79 (m, 1H), 7.57 (s, 1H), 7.55-7.51 (m, 2H), 7.11 (s, 1H), 7.05 (s, 2H), 6.96-6.88 (m, 1H), 5.37 (d, J=15.2 Hz, 1H), 4.71 (dd, J=8.4, 6.4 Hz, 1H), 4.36 (d, J=15.2 Hz, 1H), 3.42 (dd, J=15.2, 6.4 Hz, 1H), 3.36 (s, 3H), 2.89 (dd, J=15.2, 8.4 Hz, 1H).

### 2-((1H-benzo[d][1,2,3]triazol-5-yl)methyl)-3-((4-chloro-1-methyl-1H-pyrazol-3-yl)methyl) isoindolin-1-one (250)

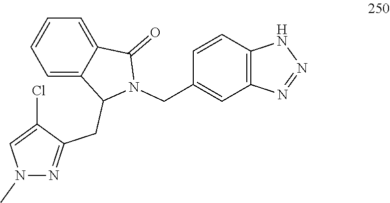 [SMILES: Cn1cc(Cl)c(CC2c3ccccc3C(=O)N2Cc2ccc3[nH]nnc3c2)n1]

The title compound 250 was prepared according to the procedure described for compound 232 as a white solid (10 mg, 6.7%). MS (EST) m/z 393 [M+H]⁺. ¹H NMR (400 MHz, DMSO-d₆) δ 15.59 (s, 1H), 8.0-7.94 (m, 1H), 7.86-7.78 (m, 2H), 7.70 (d, J=7.1 Hz, 1H), 7.56-7.44 (m, 2H), 7.25-7.20 (m, 2H), 5.31 (d, J=15.4 Hz, 1H), 4.73 (br, 1H), 4.60-4.55 (m, 1H), 3.73 (s, 3H), 3.28 (dd, J=14.8, 5.2 Hz, 1H), 2.94 (dd, J=14.8, 7.2 Hz, 1H).

### 6-((1-((4-chloro-1-methyl-1H-pyrazol-5-yl)methyl)-3-oxoisoindolin-2-yl)methyl)benzo[d]oxazol-2(3H)-one (251)

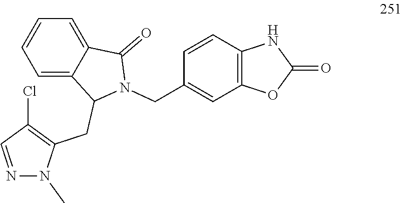 [SMILES: Cn1ncc(Cl)c1CC1c2ccccc2C(=O)N1Cc1ccc2[nH]c(=O)oc2c1]

The title compound 251 was prepared according to general procedure E as a white solid (24 mg, 16.1%). MS (ESI) m/z 409 [M+H]⁺. ¹H NMR (400 MHz, DMSO-d₆) δ 11.58 (s, 1H), 7.75-7.72 (m, 1H), 7.55 (s, 1H), 7.54-7.49 (m, 2H), 7.14 (s, 1H), 7.05-6.99 (m, 2H), 6.85 (t, J=4.8 Hz, 1H), 5.21 (d, J=15.2 Hz, 1H), 4.66 (dd, J=8.2, 6.0 Hz, 1H), 4.31 (d, J=15.2 Hz, 1H), 3.57 (s, 3H), 3.50 (dd, J=14.8, 6.0 Hz, 1H), 2.98 (dd, J=14.8, 8.2 Hz, 1H).

### 6-((1-(cyclopentylmethyl)-3-oxoisoindolin-2-yl)methyl)benzo[d]oxazol-2(3H)-one (252)

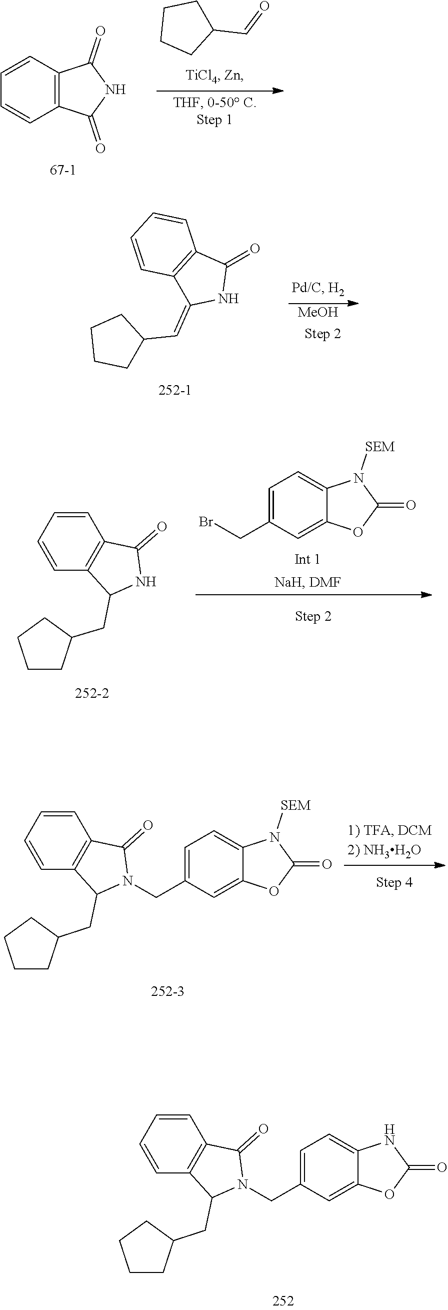

### Step 1: (E)-3-(cyclopentylmethylene)isoindolin-1-one (252-1)

To a solution of cyclopentanecarbaldehyde (500 mg, 5.1 mmol), 2H-isoindole-1,3-dione (749 mg, 5.1 mmol), and Zn (662 mg, 10.2 mmol) in THF (20 ml) was added TiCl₄ (1.93 g, 10.2 mmol) in portions at 0° C. under N₂. The reaction mixture was stirred at 50° C. for 2 h. The resulted mixture was diluted with EtOAc (30 mL), the solution was successively washed with water (30 mL) and brine (30 mL), dried over anhydrous Na₂SO₄, and after filtration, the filtrate was concentrated under reduced pressure. The residue was purified by TLC, Layered with (DCM:MeOH) (50:1) to afford the title compound (550 mg, 50%) as a white solid. MS (ESI) m/z 214 [M+H]⁺.

### Step 2:3-(cyclopentylmethyl)isoindolin-1-one (252-2)

A solution of (E)-3-(cyclopentylmethylene)isoindolin-1-one (250 mg, 1.2 mmol), Pd/C(25 mg) in MeOH (10 ml) was stirred at 25° C. for 1 h under H₂. The reaction mixture was concentrated under reduced pressure to give a residue. It was purified by flash (PE:EtOAc=1:1) to afford the title compound (200 mg, 84%) as a white solid. MS (EST) m/z 216 [M+H]⁺.

### Step 3: 6-((1-(cyclopentylmethyl)-3-oxoisoindolin-2-yl)methyl)-3-((2-(trimethylsilyl)ethoxy) methyl)benzo[d]oxazol-2(3H)-one (252-3)

To a solution of 3-(cyclopentylmethyl)-2,3-dihydroisoindol-1-one (100 mg, 0.4 mmol) in DMF (10 ml) was added NaH (22 mg, 0.5 mmol) under 0° C. The reaction mixture was stirred at 0° C. for 1h. 6-(bromomethyl)-3-((2-(trimethylsilyl)ethoxy)methyl) benzo[d]oxazol-2(3H)-one (200 mg, 0.5 mmol) was added in reaction mixture. The reaction mixture was stirred at 0° C. for 1 h. H₂O (10 mL) was added in mixture. The resulted mixture was diluted with EtOAc (30 mL), and the organic phase was successively washed with water (30 mL) and brine (30 mL), dried over anhydrous Na₂SO₄, and after filtration, the filtrate was concentrated under reduced pressure. The residue was purified by TLC, Layered with (DCM:MeOH) (50:1) to afford the title compound (150 mg, 76%) as a white solid. MS (ESI) m/z 493 [M+H]⁺.

### Step 4: 6-((1-(cyclopentylmethyl)-3-oxoisoindolin-2-yl)methyl)benzo[d]oxazol-2(3H)-one (252)

6-((1-(cyclopentylmethyl)-3-oxoisoindolin-2-yl)methyl)-3-((2-(trimethylsilyl)ethoxy)methyl) benzo[d]oxazol-2(3H)-one (150 mg, 0.3 mmol) was dissolved in DCM (5 mL), TFA (5 ml) was added dropwise at 0° C. The resulting mixture was stirred for additional 0.5 h at 0° C. LC-MS and TLC showed disappearance of the starting material. The resulting mixture was concentrated under reduced pressure. NH₄OH (5 mL) was add dropwise in the mixture under 0° C. The resulting mixture was stirred for additional 0.5 h at 0° C. The mixture was concentrated under reduced pressure. The residue was purified by TLC, Layered with (DCM:MeOH) (50:1) to afford the title compound (52 mg, 47%) as a white solid. MS (ESI) m/z 363 [M+H]⁺. ¹H NMR (400 MHz, DMSO-d₆) δ 11.61 (s, 1H), 7.71 (d, J=7.5 Hz, 1H), 7.60-7.55 (m, 2H), 7.51-7.47 (m, 1H), 7.24 (s, 1H), 7.10-7.02 (m, 2H), 5.08 (d, J=15.1 Hz, 1H), 4.46 (t, J=4.5 Hz, 1H), 4.32 (d, J=15.1 Hz, 1H), 2.08-1.97 (m, 2H), 1.51-1.39 (m, 4H), 1.33-1.22 (m, 2H), 1.17-1.09 (m, 1H), 1.06-0.99 (m, 11H), 0.83-0.73 (m, 1H).

### 4-((3-oxo-2-((2-oxo-2,3-dihydrobenzo[d]oxazol-6-yl)methyl)isoindolin-1-yl)methyl) benzamide (253)

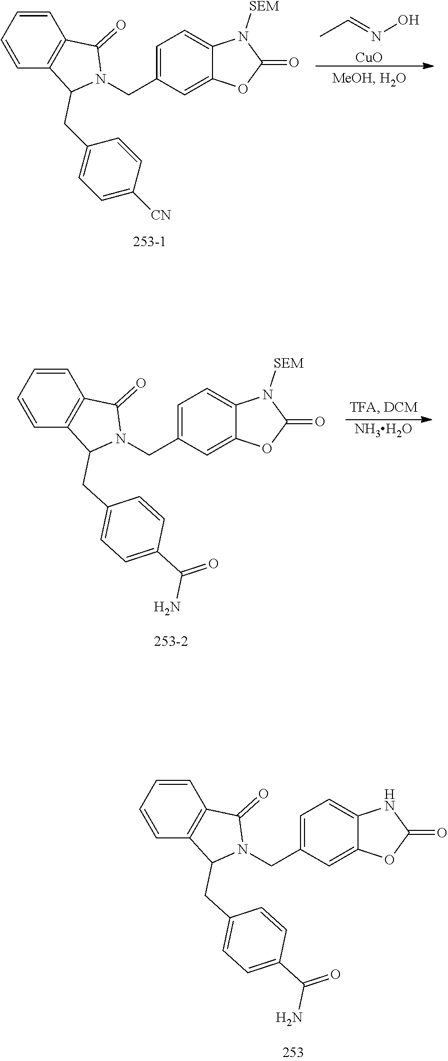

### Step 1. 4-((3-oxo-2-((2-oxo-3-((2-(trimethylsilyl)ethoxy)methyl)-2,3-dihydrobenzo[d]oxazol-6-yl)methyl)isoindolin-1-yl)methyl)benzamide (253-2)

To a solution of 4-((3-oxo-2-((2-oxo-3-((2-(trimethylsilyl)ethoxy)methyl)-2,3-dihydrobenzo[d]oxazol-6-yl)methyl)isoindolin-1-yl)methyl)benzonitrile (100 mg, 0.11 mmol) in MeOH/H₂O (10 mL) were added (E)-acetaldehyde oxime (50 mg, 0.33 mmol) and CuO (40 mg, 0.33 mmol) at r.t. The mixture was stirred for 16 h at 100° C. The mixture was diluted with EtOAc, and washed with water. The organic layer was separated, dried over anhydrous Na₂SO₄ and concentrated under vacuum. The residue was purified by silica gel flash column chromatography (PE/EtOAc=1/2) to afford 4-((3-oxo-2-((2-oxo-3-((2-(trimethylsilyl)ethoxy) methyl)-2,3-dihydrobenzo[d]oxazol-6-yl)methyl)isoindolin-1-yl)methyl)benzamide (60 mg, 60%) as a yellow oil. MS (ESI) m/z 544 [M+H]⁺.

### Step 2. 4-((3-oxo-2-((2-oxo-2,3-dihydrobenzo[d]oxazol-6-yl)methyl)isoindolin-1-yl)methyl) benzamide (253)

To a solution of 4-((3-oxo-2-((2-oxo-3-((2-(trimethylsilyl)ethoxy)methyl)-2,3-dihydrobenzo[d]oxazol-6-yl)methyl)isoindolin-1-yl)methyl)benzamide (60 mg, 0.12 mmol) in DCM (5 mL) were added TFA (2 mL), the mixture was stirred for 1 h at r.t. The mixture was concentrated in vacuo and then dissolved in NH₄OH (2 ml), then mixture reaction was stirred for another 1 h at r.t., and purified by prep-HPLC to give 4-((3-oxo-2-((2-oxo-2,3-dihydrobenzo[d]oxazol-6-yl)methyl)isoindolin-1-yl)methyl)benzamide (8 mg, 17.5% o) as a white solid. MS (ESI) m/z 414 [M+H]⁺. ¹H NMR (400 MHz, CD₃OD) δ 7.68-7.62 (m, 3H), 7.53 (td, J=7.5, 1.2 Hz, 1H), 7.45 (d, J=7.4 Hz, 1H), 7.34 (d, J=7.5 Hz, 1H), 7.16 (s, 1H), 7.11 (dd, J=8.1, 1.6 Hz, 1H), 7.07-7.03 (in, 3H), 5.29 (d, J=15.2 Hz, 1H), 4.82 (d, J=5.5 Hz, 1H), 4.51 (d, J=15.2 Hz, 1H), 3.42 (d, J=4.3 Hz, 1H), 3.32-3.27 (in, 1H). 3-((3-bromopyridin-2-yl)methyl)-2-((2-methoxy-1H-benzo[d]imidazol-5-yl)methyl) isoindolin-1-one (254)

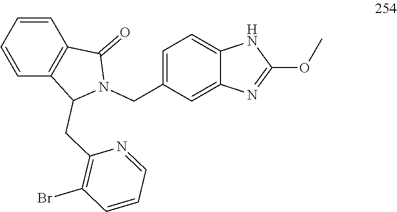 [SMILES: COc1nc2cc(CN3C(=O)c4ccccc4C3Cc3ncccc3Br)ccc2[nH]1]

The title compound 254 was prepared according to general procedure F as a white solid (27 mg, 21.3%). MS (EST) m/z 463 [M+H]⁺. ¹H NMR (400 MHz, DMSO-d₆) δ 11.81-11.72 (m, LH), 8.54 (dd, J=4.4, 1.6 Hz, 1H), 8.09-8.02 (m, 1H), 7.77-7.71 (m, 1H), 7.51-7.43 (m, 2H), 7.33-7.22 (m, 2H), 7.15-7.09 (m, 11H), 7.01-6.88 (m, 2H), 6.83-6.76 (m, 1H), 5.24-5.13 (m, 1H), 5.10-5.03 (m, 11H), 4.36 (dd, J=15.2, 8.4 Hz, 1H), 4.01 (d, J=2.4 Hz, 3H), 3.70-3.58 (m, 1H), 3.22-3.11 (in, 1H).

The intermediate 254-4 was prepared as follows:

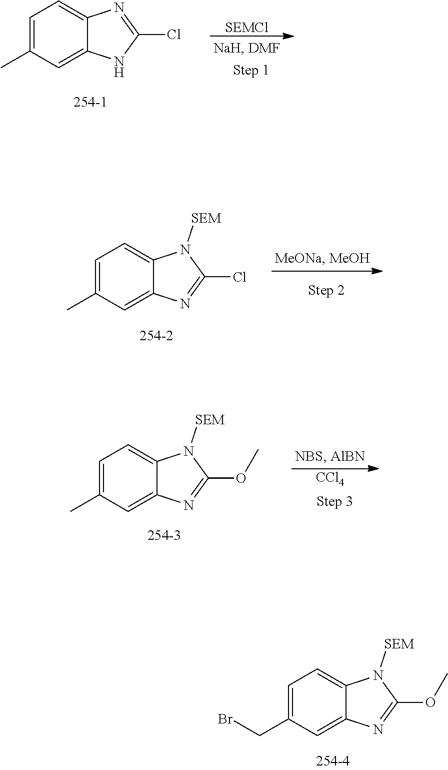

Step 1 was performed according to procedure outlined for step 2 for the preparation of Int 1.

Step 2. 2-[(2-methoxy-6-methyl-benzimidazol-1-yl)methoxy]ethyl-trimethyl-silane (254-3)

To a solution of 2-[(2-chloro-6-methyl-benzimidazol-1-yl)methoxy]ethyl-trimethyl-silane (1 g, 3.37 mmol) in CH₃OH (5.0 mL) was added CH₃ONa (5.4M/L, 5 mL) at r.t. under N₂. After addition, the mixture was stirred at 80° C. for 1 hi. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with flash (PE/EtOAc=1:1) to give the product 2-[(2-methoxy-6-methyl-benzimidazol-1-yl)methoxy]ethyl-trimethyl-silane (800 mg, 81.2%) as a yellow oil.

Step 3 was performed according to procedure outlined for step 3 for the preparation of Int 1.

### 3-((3-bromopyridin-2-yl)methyl)-2-((2-chloro-1H-benzo[d]imidazol-5-yl)methyl) isoindolin-1-one (255)

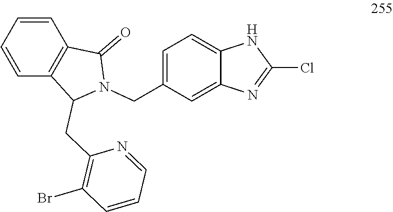 [SMILES: O=C1c2ccccc2C(Cc2ncccc2Br)N1Cc1ccc2[nH]c(Cl)nc2c1]

The title compound 255 was prepared according to the procedure described for 254 as a white solid (48 mg, 57.6%). MS (ESI) m/z 467 [M+H]⁺. ¹H NMR (400 MHz, DMSO-d₆) δ 13.17 (s, 1H), 8.54 (dd, J=4.6, 1.6 Hz, 1H), 8.03 (dd, J=8.0, 1.8 Hz, 1H), 7.79-7.73 (m, 1H), 7.56-7.27 (m, 3H), 7.29 (dd, J=8.0, 4.4 Hz, 1H), 7.22 (s, 1H), 7.01-6.96 (m, 2H), 5.19 (d, J=15.2 Hz, 1H), 5.11 (t, J=6.8 Hz, 1H), 4.47 (d, J=15.2 Hz, 1H), 3.62 (dd, J=14.8, 6.0 Hz, 1H), 3.19 (dd, J=14.8, 7.2 Hz, 1H).

### 3-Amino-6-((1-((3-bromopyridin-2-yl)methyl)-3-oxoisoindolin-2-yl)methyl)benzo[d]oxazol-2(3H)-one (256)

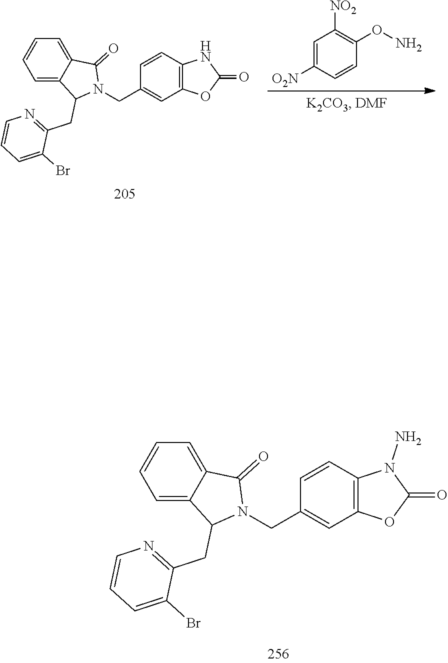 [SMILES: O=C1c2ccccc2C(Cc2ncccc2Br)N1Cc1ccc2[nH]c(=O)oc2c1 ; NOc1ccc([N+](=O)[O-])cc1[N+](=O)[O-] ; Nn1c(=O)oc2cc(CN3C(=O)c4ccccc4C3Cc3ncccc3Br)ccc21]

To a solution of 6-((1-((3-bromopyridin-2-yl)methyl)-3-oxoisoindolin-2-yl)methyl)benzo[d]oxazol-2(3H)-one (32 mg, 0.07 mmol) in DMF (3 mL) was added 0-(2,4-dinitrophenyl)hydroxylamine (14 mg, 0.07 mmol) and K₂CO₃ (30 mg, 0.21 mmol). Then the mixture was stirred at r.t. overnight. The reaction progress was monitored by LC/MS. Once completed, the solvent was removed under reduced pressure. The obtained residue was dissolved in DCM (20 mL) and washed with water (20 mL, 3 times). The organic layer was dried over MgSO₄, filtered and concentrated under reduced pressure. The residue was purified by prep-HPLC to give the product as a yellow solid (1 mg, 2.7%). MS (ESI) m/z 465 [M+H]⁺.

### 2-((3-oxo-2-((2-oxo-2,3-dihydrobenzo[d]oxazol-6-yl)methyl)isoindolin-1-yl)methyl) nicotinonitrile (257)

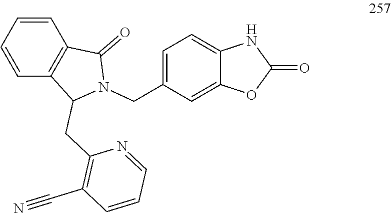 [SMILES: N#Cc1cccnc1CC1c2ccccc2C(=O)N1Cc1ccc2[nH]c(=O)oc2c1]

The title compound 257 was prepared according to general procedure E as a white solid (71 mg, 59%). MS (ESI) m/z 397 [M+H]⁺. ¹H NMR (400 MHz, DMSO-d₆) δ 11.60 (bs, 1H), 8.76 (d, J=4.9 Hz, 1H), 8.17 (d, J=7.9 Hz, 1H), 7.70 (d, J=7.3 Hz, 1H), 7.55-7.44 (m, 3H), 7.17 (d, J=7.3 Hz, 1H), 7.05 (d, J=1.5 Hz, 1H), 6.99 (d, J=8.0 Hz, 1H), 6.92 (dd, J=8.0, 1.6 Hz, 1H), 5.16 (t, J=6.0 Hz, 1H), 4.99 (d, J=15.6 Hz, 1H), 4.53 (d, J=15.6 Hz, 1H), 3.64 (dd, J=15.2, 5.6 Hz, 1H), 3.40 (dd, J=15.2, 6.4 Hz, 1H).

### (R)-2-((3-oxo-2-((2-oxo-2,3-dihydrobenzo[d]oxazol-6-yl)methyl)isoindolin-1-yl)methyl) nicotinonitrile (258) and (S)-2-((3-oxo-2-((2-oxo-2,3-dihydrobenzo[d]oxazol-6-yl)methyl)isoindolin-1-yl)methyl)nicotinonitrile (259)

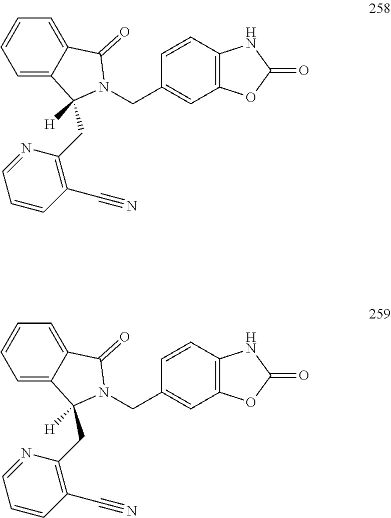 [SMILES: N#Cc1cccnc1C[C@@H]1c2ccccc2C(=O)N1Cc1ccc2[nH]c(=O)oc2c1 ; N#Cc1cccnc1C[C@H]1c2ccccc2C(=O)N1Cc1ccc2[nH]c(=O)oc2c1]

Chiral separation of 257 by chiral prep-HPLC (column: CHIRALPAK IF; column size: 2 cm×25 cm, 5 μm; mobile phase A: MTBE; mobile phase B: MeOH:DCM) was conducted to afford two enantiomers: compound 258 and compound 259. One of the two enantiomers was a white solid, with RT=1.809 min (CHIRAL HPLC, column: CHIRALPAK IF-3; column size: 4.6*50 mm, 3 μm; mobile phase: MTBE (0.1% DEA):(MeOH:DCM=1:1)=80:20; flow: 1.0 mL/min). MS (ESI) m/z 397 [M+H]⁺. ¹H NMR (400 MHz, DMSO-d₆) δ 11.60 (s, 1H), 8.76 (dd, J=4.9, 1.7 Hz, 1H), 8.17 (dd, J=7.9, 1.7 Hz, 1H), 7.70 (d, J=7.3 Hz, 1H), 7.53 (td, J=7.4, 1.6 Hz, 1H), 7.48 (dt, J=7.9, 5.1 Hz, 2H), 7.17 (d, J=7.3 Hz, 1H), 7.05 (d, J=1.5 Hz, 1H), 6.99 (d, J=8.0 Hz, 1H), 6.92 (dd, J=8.0, 1.6 Hz, 1H), 5.16 (t, J=6.0 Hz, 1H), 4.99 (d, J=15.5 Hz, 1H), 4.53 (d, J=15.5 Hz, 1H), 3.64 (dd, J=15.2, 5.7 Hz, 1H), 3.40 (dd, J=15.2, 6.4 Hz, 1H). The other one of the two enantiomers was a white solid, with RT-2.246 min, (CHIRAL HPLC, column: CHIRALPAK IF-3; column size: 4.6*50 mm, 3 μm; mobile phase: MTBE (0.1% DEA):(MeOH:DCM=1:1)=80:20; flow: 1.0 mL/min). MS (ESI) m/z 397 [M+H]⁺. ¹H NMR (400 MHz, DMSO-d₆) δ 11.60 (s, 1H), 8.76 (dd, J=4.9, 1.7 Hz, 1H), 8.17 (dd, J=7.9, 1.7 Hz, 1H), 7.70 (d, J=7.3 Hz, 1H), 7.53 (td, J=7.4, 1.6 Hz, 1H), 7.48 (dt, J=7.9, 5.1 Hz, 2H), 7.17 (d, J=7.3 Hz, 1H), 7.05 (d, J=1.5 Hz, 1H), 6.99 (d, J=8.0 Hz, 1H), 6.92 (dd, J=8.0, 1.6 Hz, 1H), 5.16 (t, J=6.0 Hz, 1H), 4.99 (d, J=15.5 Hz, 1H), 4.53 (d, J=15.5 Hz, 1H), 3.64 (dd, J=15.2, 5.6 Hz, 1H), 3.40 (dd, J=15.2, 6.4 Hz, 1H).

### 6-((1-(((6-chloropyridin-2-yl)oxy)methyl)-3-oxoisoindolin-2-yl)methyl)benzo[d]oxazol-2(3H)-one (260)

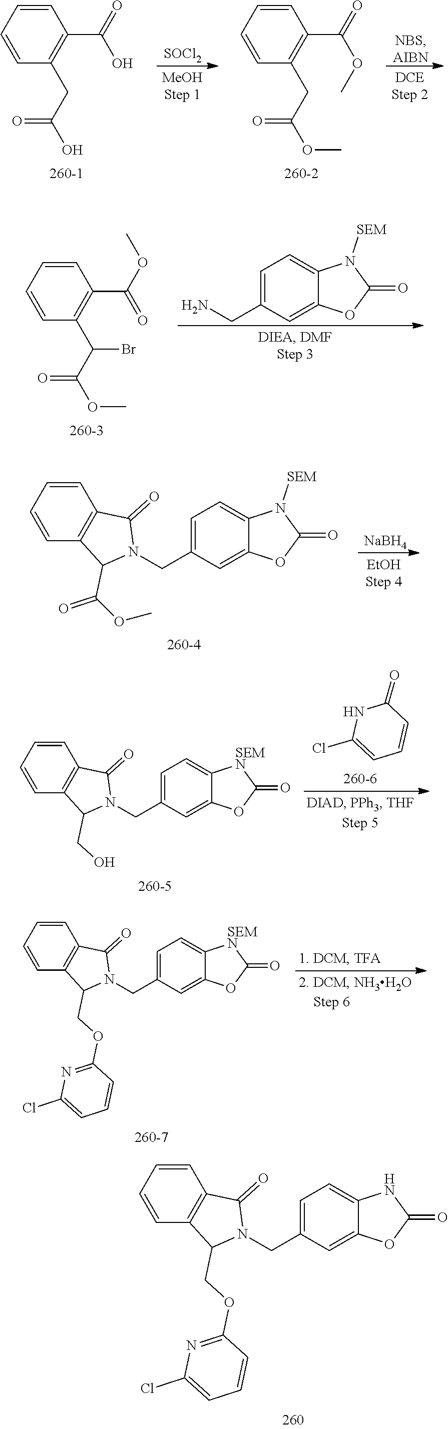

### Step 1. methyl 2-(2-methoxy-2-oxoethyl)benzoate (260-2)

2-(carboxymethyl)benzoic acid (2 g, 11.1 mmol) was dissolved in methanol (20 mL) and thionyl chloride (1 mL) was added dropwise. The solution was heated under reflux for 2 h and concentrated in vacuo. The residue was dissolved in EtOAc and washed several times with 10% NaHCO₃. After drying over Na₂SO₄, the solvent was evaporated in vacuo to give methyl 2-(2-methoxy-2-oxoethyl)benzoate (2.1 g) as a yellow oil, which was used in the next step without further purification. MS (ESI) m/z 209 [M+H]⁺.

### Step 2. methyl 2-(1-bromo-2-methoxy-2-oxoethyl)benzoate (260-3)

To a solution of methyl 2-(2-methoxy-2-oxoethyl)benzoate (2.1 g, 10.1 mmol) in DCE (20 mL) were added NBS (1.97 g, 11.1 mmol) and AIBN (165 mg, 1.01 mmol) at r.t. The mixture was stirred for 14 h at 80° C. After the reaction was completed, the solid was filtered, and the filtrate was concentrated. The residue was purified by silica gel flash column chromatography (PE/EA=10/1) to afford methyl 2-(1-bromo-2-methoxy-2-oxoethyl)benzoate (1.5 g, 51%) as a yellow oil. MS (ESI) m/z 287 [M+H]⁺.

### Step 3. methyl 3-oxo-2-((2-oxo-3-((2-(trimethylsilyl)ethoxy)methyl)-2,3-dihydrobenzo[d]oxazol-6-yl)methyl)isoindoline-1-carboxylate (260-4)

To a solution of 6-(aminomethyl)-3-((2-(trimethylsilyl)ethoxy)methyl)benzo[d]oxazol-2(3H)-one (400 mg, 1.36 mmol) and DIEA (0.47 mL, 2.72 mmol) in DMF (4 mL) was added methyl 2-(1-bromo-2-methoxy-2-oxoethyl)benzoate (390 mg, 1.36 mmol) under N₂. The mixture was stirred for 2 h at 80° C., the mixture was purified by C18 Column (ACN=90%) to give methyl 3-oxo-2-((2-oxo-3-((2-(trimethylsilyl)ethoxy)methyl)-2,3-dihydrobenzo[d]oxazol-6-yl)methyl) isoindoline-1-carboxylate (200 mg, 31%) as a white solid. MS (ESI) m/z 469 [M+H]⁺.

### Step 4. 6-((1-(hydroxymethyl)-3-oxoisoindolin-2-yl)methyl)-3-((2-(trimethylsilyl)ethoxy) methyl)benzo[d]oxazol-2(3H)-one (260-5)

To a solution of methyl 3-oxo-2-((2-oxo-3-((2-(trimethylsilyl)ethoxy)methyl)-2,3-dihydrobenzo[d]oxazol-6-yl)methyl)isoindoline-1-carboxylate (200 mg, 0.42 mmol) in ethanol (2 mL) was added NaBH₄ (49 mg, 1.28 mmol) under N₂, and stirred for 12 h, the mixture was concentrated under vacuum. The residue was purified by silica gel column chromatography (PE/EtOAc=1/1) to give 6-((1-(hydroxymethyl)-3-oxoisoindolin-2-yl)methyl)-3-((2-(trimethylsilyl)ethoxy)methyl)benzo[d]oxazol-2(3H)-one (171 mg, 90%) as a white solid. MS (ESI) m/z 441 [M+H]⁺.

### Step 5. 6-((1-(((6-chloropyridin-2-yl)oxy)methyl)-3-oxoisoindolin-2-yl)methyl)-3-((2-(trimethylsilyl)ethoxy)methyl)benzo[d]oxazol-2(3H)-one (260-7)

To a solution of 6-((1-(hydroxymethyl)-3-oxoisoindolin-2-yl)methyl)-3-((2-(trimethylsilyl) ethoxy)methyl)benzo[d]oxazol-2(3H)-one (57 mg, 0.12 mmol), 6-chloropyridin-2(1H)-one (17 mg, 0.12 mmol) and PPh₃ (41 mg, 0.15 mmol) in THF (2 mL) was added DIAD (31 mg, 0.15 mmol) under N₂. The mixture was stirred at r.t. for 14 h. The solution was purified by prep TLC (PE/EtOAc=3/1) to give 6-((1-(((6-chloropyridin-2-yl)oxy)methyl)-3-oxoisoindolin-2-yl)methyl)-3-((2-(trimethylsilyl) ethoxy)methyl)benzo[d]oxazol-2(3H)-one (20 mg, 28%) as a white solid. MS (ESI) m/z 552 [M+H]⁺.

### Step 6. 6-((1-(((6-chloropyridin-2-yl)oxy)methyl)-3-oxoisoindolin-2-yl)methyl)benzo[d]oxazol-2(3H)-one (260)

To a solution of 6-((1-(((6-chloropyridin-2-yl)oxy)methyl)-3-oxoisoindolin-2-yl)methyl)-3-((2-(trimethylsilyl)ethoxy)methyl)benzo[d]oxazol-2(3H)-one (20 mg, 0.03 mmol) in DCM (3 mL) was added TFA (1 mL). The mixture was stirred for 3 h and then concentrated under vacuum. The residue was taken in DCM (3 mL) and ammonium hydroxide (1 mL) was added at 0° C. This suspension was allowed to stir for an additional 1 h. The mixture was concentrated and purified by prep HPLC to give 6-((1-(((6-chloropyridin-2-yl)oxy)methyl)-3-oxoisoindolin-2-yl)methyl)benzo[d]oxazol-2(3H)-one (3 mg, 18%) as a white solid. MS (ESI) m/z 422 [M+H]⁺. ¹H NMR (400 MHz, CD₃OD) δ 7.92-7.82 (m, 1H), 7.72-7.61 (m, 2H), 7.61-7.47 (m, 2H), 7.19-7.08 (m, 2H), 6.80-6.75 (m, 2H), 6.43 (d, J=8.1 Hz, 1H), 5.01-4.92 (m, 2H), 4.84-4.82 (m, 1H), 4.78 (dd, J=11.8, 3.4 Hz, 1H), 4.54 (dd, J=11.9, 4.0 Hz, 1H).

### 6-((1-((4-methylpyridazin-3-yl)methyl)-3-oxoisoindolin-2-yl)methyl)benzo[d]oxazol-2(3H)-one (261)

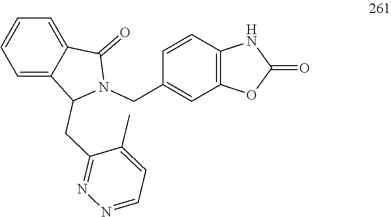 [SMILES: Cc1ccnnc1CC1c2ccccc2C(=O)N1Cc1ccc2[nH]c(=O)oc2c1]

The title compound 261 was prepared according to general procedure E as a white solid (3 mg, 2.3%). MS (ESI) m/z 387 [M+H]⁺. ¹H NMR (400 MHz, DMSO-d₆) δ 11.59 (bs, 1H), 8.98 (d, J=5.1 Hz, 1H), 7.82-7.67 (m, 1H), 7.57-7.30 (m, 3H), 7.22-6.84 (m, 4H), 5.30 (dd, J=7.4, 5.6 Hz, 1H), 5.04 (d, J=15.4 Hz, 1H), 4.49 (d, J=15.4 Hz, 1H), 3.64 (dd, J=15.7, 5.6 Hz, 1H), 3.21 (dd, J=15.7, 7.5 Hz, 1H), 2.08 (s, 3H).

The intermediate 261-4 was prepared as follows:

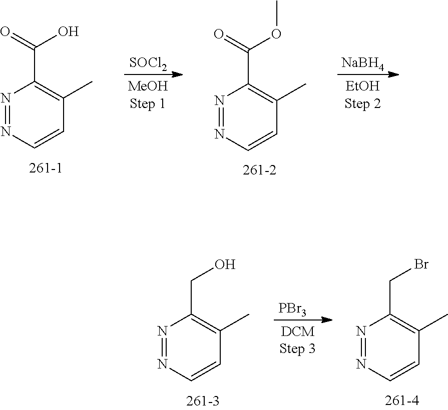

### Step 1. methyl 4-methylpyridazine-3-carboxylate (261-2)

To a solution of 4-methylpyridazine-3-carboxylic acid (100 mg, 0.73 mmol) in MeOH (5 mL) was added SOCl₂ (1 mL) at 0° C. for 2 h. The mixture was concentrated under vacuum to afford the title compound (100 mg, 90.1%) as a yellow oil. MS (ESI) m/z 153 [M+H]⁺.

### Step 2. (4-methylpyridazin-3-yl)methanol (261-3)

To a solution of methyl 4-methylpyridazine-3-carboxylate (100 mg, 0.66 mmol) in EtOH (5 mL) was added NaBH₄ (83 mg, 2.17 mmol) at 0° C. and reacted at r.t. for 2 h. The mixture was diluted with EA (30 mL) and washed with water (3×20 mL). The organic layer was separated, dried over anhydrous Na₂SO₄ and concentrated under vacuum to afford the title compound (60 mg, 73.5%) as a yellow oil. MS (ESI) m/z 125 [M+H]⁺.

### Step 3. 3-(bromomethyl)-4-methylpyridazine (261-4)

To a solution of (4-methylpyridazin-3-yl)methanol (60 mg, 0.48 mmol) in DCM (5 mL) was added PBr₃ (191 mg, 0.58 mmol) at 0° C. and stirred for 2 h at r.t. The reaction mixture was quenched with water and extracted with EA (2×30 mL) and concentrated to afford the title compound (60 mg, 66.6%) as a light yellow solid. MS (ESI) m/z 187/189 [M+H]⁺.

### 6-((1-((5-bromopyridin-2-yl)methyl)-3-oxoisoindolin-2-yl)methyl)benzo[d]oxazol-2(3H)-one (262)

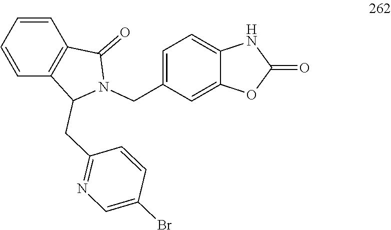 [SMILES: O=C1c2ccccc2C(Cc2ccc(Br)cn2)N1Cc1ccc2[nH]c(=O)oc2c1]

The title compound 262 was prepared according to general procedure E as a white solid (11 mg, 14%). MS (ESI) m/z 450/452 [M+H]⁺. ¹H NMR (400 MHz, CD₃OD) δ 8.49 (d, J=2.1 Hz, 1H), 7.70 (dd, J=1 3.6, 5.1 Hz, 2H), 7.54 (d, J=7.5 Hz, 1H), 7.48 (d, J=7.4 Hz, 1H), 7.28 (d, J=7.6 Hz, 1H), 7.09 (s, 1H), 7.02 (t, J=6.3 Hz, 2H), 6.85 (d, J=8.3 Hz, 1H), 5.21 (d, J=15.4 Hz, 1H), 5.00 (t, J=5.5 Hz, 1H), 4.54 (d, J=15.4 Hz, 1H), 3.44 (dd, J=14.3, 4.9 Hz, 1H).

### 6-((1-((2-chloro-4-methylpyridin-3-yl)methyl)-3-oxoisoindolin-2-yl)methyl)benzo[d]oxazol-2(3H)-one (263)

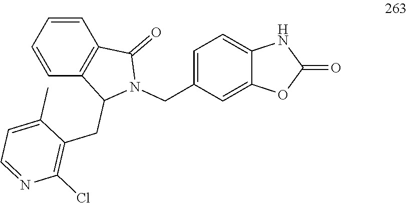 [SMILES: Cc1ccnc(Cl)c1CC1c2ccccc2C(=O)N1Cc1ccc2[nH]c(=O)oc2c1]

The title compound 263 was prepared according to general procedure E as a yellow solid (3 mg, 18%). MS (ESI) m/z 420 [M+H]⁺. ¹H NMR (400 MHz, CD₃OD) δ 8.20 (d, J=5.0 Hz, 1H), 7.87 (d, J=7.6 Hz, 1H), 7.52 (t, J=7.5 Hz, 1H), 7.42 (t, J=7.5 Hz, 1H), 7.26 (d, J=5.0 Hz, 1H), 7.10 (s, 1H), 7.04 (s, 2H), 6.60 (d, J=7.6 Hz, 1H), 5.38 (d, J=15.2 Hz, 1H), 4.89-4.77 (m, 1H), 4.36 (d, J=15.2 Hz, 1H), 3.63 (dd, J=13.8, 6.4 Hz, 1H), 2.89 (dd, J=13.8, 9.3 Hz, 1H), 2.03 (s, 3H).

The intermediate 263-3 was prepared as follows:

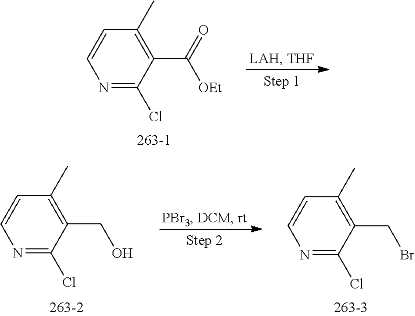

### Step 1. (2-chloro-4-methylpyridin-3-yl)methanol (263-2)

To a solution of ethyl 2-chloro-4-methylnicotinate (2.1 g, 10 mmol) in THF (30 mL) was added LAH (800 mg, 16 mmol) in portions at 0° C., then stirred for 2 h at r.t. Cooled to 0° C., the reaction mixture was carefully quenched with 0.8 ml of water, 0.8 mL of 15% NaOH (aq.) (2 mL) and 2.4 mL of water. Then the reaction mixture was added MgSO₄, stirred for 15 min. The mixture was filtered, and the filtrate was concentrated. The residue was purified by column chromatography on silica gel (eluting (Petroleum ether:EtOAc=3:1 to 1:1) to give (2-chloro-4-methylpyridin-3-yl)methanol (800 mg, 51%) as a white solid. MS (ESI) m/z 158[M+H]⁺.

### Step 2. 3-(bromomethyl)-2-chloro-4-methylpyridine (263-3)

To a solution of (2-chloro-4-methylpyridin-3-yl)methanol (450 mg, 2.87 mmol) in dried DCM (5 mL) was added dropwise PBr₃ (1.5 g, 5.74 mmol) at 0° C. The mixture was stirred for 1 h. The reaction mixture was quenched with NaHCO₃ (aq.). The organic layer was separated, dried over Na₂SO₄ and concentrated to give 3-(bromomethyl)-2-chloro-4-methylpyridine (550 mg, 87%) as a yellow oil. MS (ESI) m/z 220/222 [M+H]⁺.

### 3-((3-oxo-2-((2-oxo-2,3-dihydrobenzo[d]oxazol-6-yl)methyl)isoindolin-1-yl)methyl) benzamide (264)

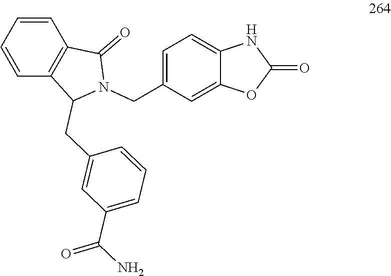 [SMILES: NC(=O)c1cccc(CC2c3ccccc3C(=O)N2Cc2ccc3[nH]c(=O)oc3c2)c1]

The title compound 264 was prepared according to the procedure described for compound 253 as a white solid (10 mg, 75%). MS (ESI) m/z 413 [M+H]⁺. ¹H NMR (400 MHz, DMSO-d₆) δ 7.82 (s, 1H), 7.65 (d, J=7.7 Hz, 1H), 7.59 (d, J=7.0 Hz, 2H), 7.55-7.49 (m, 1H), 7.45-7.40 (m, 1H), 7.30 (d, J=7.7 Hz, 2H), 7.24-7.17 (m, 2H), 7.10-7.02 (m, 4H), 5.14 (d, J=15.2 Hz, 1H), 4.80-4.72 (m, 1H), 4.45 (d, J=15.1 Hz, 1H), 3.44-3.37 (m, 1H), 3.25-3.16 (m, 1H).

### 6-((5-chloro-3-methyl-1-oxoisoindolin-2-yl)methyl)benzo[d]oxazol-2(3H)-one (265)

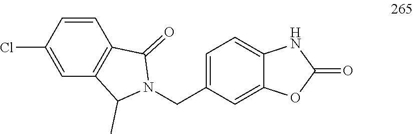 [SMILES: CC1c2cc(Cl)ccc2C(=O)N1Cc1ccc2[nH]c(=O)oc2c1]

The title compound 265 was prepared according to the procedure described for compound 48 as a white solid (15 mg, 23%). MS (ESI) m/z 329 [M+H]⁺. ¹H NMR (400 MHz, CD₃OD) 6.7.77 (d, J=8.1 Hz, 1H), 7.59 (d, J=1.8 Hz, 1H), 7.52 (d, J=8.1, 1.8 Hz, 1H), 7.21 (s, 1H), 7.15 (d, J=8.1 Hz, 1H), 7.04 (d, J=8.0 Hz, 1H), 5.13 (d, J=15.3 Hz, 1H), 4.54-4.38 (m, 2H), 1.45 (d, J=6.8 Hz, 3H).

### 3-((3-bromopyridin-2-yl)methyl)-2-((2-oxo-1,2-dihydropyridin-4-yl)methyl)isoindolin-1-one (266)

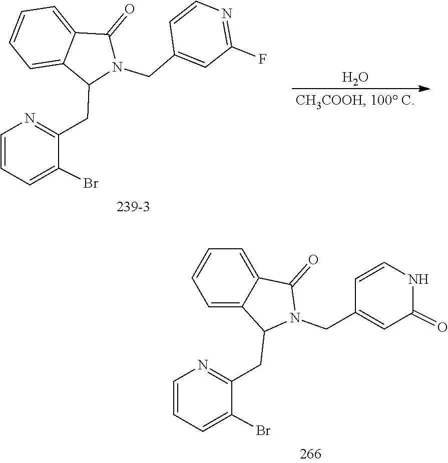 [SMILES: O=C1c2ccccc2C(Cc2ncccc2Br)N1Cc1ccnc(F)c1 ; O ; O=C1c2ccccc2C(Cc2ncccc2Br)N1Cc1cc[nH]c(=O)c1]

To a solution of 3-((3-bromopyridin-2-yl)methyl)-2-((2-fluoropyridin-4-yl)methyl)isoindolin-1-one (22 mg, 0.05 mmol) in AcOH (1 mL) was added H₂O (0.1 mL) and the mixture was stirred at 100° C. overnight. The reaction progress was monitored by LC-MS. Once completed, the solvent was removed under reduced pressure and the residue was purified by prep-HPLC to give the product as a white solid (11 mg, 50%). MS (ESI) m/z 410 [M+H]⁺. ¹H NMR (400 MHz, DMSO-d₆) δ 11.44 (bs, 1H), 8.56 (dd, J=4.6, 1.5 Hz, 1H), 8.05 (dd, J=8.1, 1.5 Hz, 1H), 7.78-7.70 (m, 1H), 7.52 (m, 2H), 7.32-7.21 (m, 2H), 7.16-7.07 (m, 1H), 5.96-5.78 (m, 2H), 5.25 (t, J=6.5 Hz, 1H), 4.77 (d, J=16.4 Hz, 1H), 4.30 (d, J=16.4 Hz, 1H), 3.55 (dd, J=15.2, 6.1 Hz, 1H), 3.24 (dd, J=15.2, 7.0 Hz, 1H).

### 3-((3-bromopyridin-2-yl)methyl)-2-((5-hydroxypyridin-2-yl)methyl)isoindolin-1-one (267)

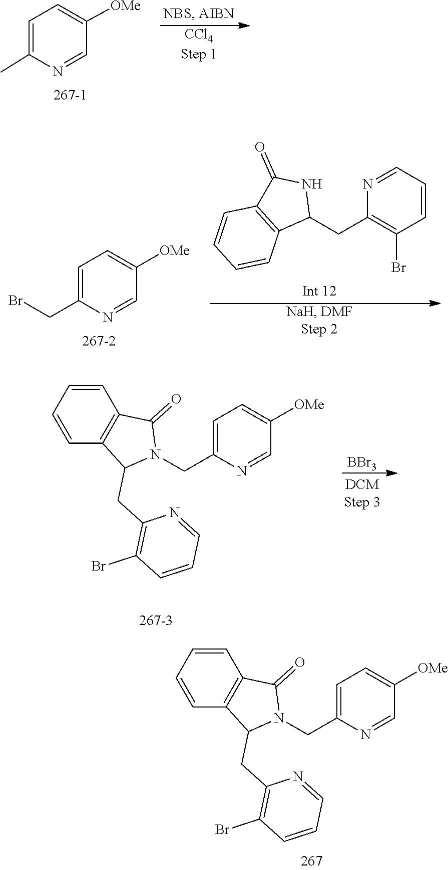

### Step 1. 5-(bromomethyl)-2-methoxy-pyridine (267-2)

To a solution of 2-methoxy-5-methyl-pyridine (500 mg, 4.06 mmol), NBS (0.76 g, 4.26 mmol) in CCl₄ (20 mL), was added AIBN (67 mg, 0.41 mmol) at r.t. under N₂. After addition, the mixture was stirred at 90° C. with microwave for 1 h. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with flash (PE/EtOAc=3:1) to give the product 5-(bromomethyl)-2-methoxy-pyridine (350 mg, 42.7%) as a yellow oil.

### Step 2. 3-[(3-bromo-2-pyridyl)methyl]-2-[(5-methoxy-2-pyridyl)methyl]isoindolin-1-one (267-3)

To a solution of 3-[(3-bromo-2-pyridyl)methyl]isoindolin-1-one (178 mg, 0.59 mmol) in DMF (2 mL), was added NaH (28 mg, 0.71 mmol) at 0° C. under N₂. After addition, the mixture was stirred at 0° C. for 1 h, and then added a solution of 2-(bromomethyl)-5-methoxypyridine (119 mg, 0.59 mmol) in DMF (1 mL) at 0° C. under N₂. After addition, the mixture was stirred at 25° C. for 1 h. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified with flash (PE/EtOAc=1:1) to give the product 3-[(3-bromo-2-pyridyl)methyl]-2-[(5-methoxy-2-pyridyl)methyl]isoindolin-1-one (150 mg, 60.2%) as a yellow oil. MS (ESI) m/z 424 [M+H]⁺.

### Step 3. 3-((3-bromopyridin-2-yl)methyl)-2-((5-hydroxypyridin-2-yl)methyl)isoindolin-1-one (267)

To a solution of 3-[(3-bromo-2-pyridyl)methyl]-2-[(5-methoxy-2-pyridyl)methyl]isoindolin-1-one (95 mg, 0.22 mmol) in DCM (1 mL) was added BBr₃ (18 mg, 0.45 mmol) at 0° C. under N₂. After addition, the mixture was up to r.t. and stirred for 1 h. LC-MS showed the starting material was mostly converted to the product. Then the reaction mixture was added water (10 mL) and extracted with EtOAc (10 mL×3). The organics were then combined and dried (Na₂SO₄) before concentration to dryness. The crude was purified by HPLC to give 3-((3-bromopyridin-2-yl)methyl)-2-((5-hydroxypyridin-2-yl)methyl)isoindolin-1-one (70 mg, 76.2%) as a white solid. MS (ESI) m/z 410 [M+H]⁺. ¹H NMR (400 MHz, DMSO-d₆) δ 10.10 (s, 1H), 8.50 (d, J=4.4 Hz, 1H), 8.02 (d, J=7.8 Hz, 2H), 7.80-7.61 (m, 1H), 7.56-7.39 (m, 2H), 7.30-7.16 (m, 2H), 7.16-6.95 (m, 2H), 5.27 (m, 1H), 5.03 (d, J=16.0 Hz, 1H), 4.49 (d, J=16.8 Hz, 1H), 3.59 (dd, J=15.0, 5.8 Hz, 1H), 3.28-3.08 (m, 1H).

### 2-((1H-benzo[d]imidazol-5-yl)methyl)-3-((3-bromopyridin-2-yl)methyl)isoindolin-1-one (268)

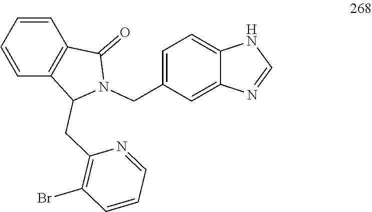 [SMILES: O=C1c2ccccc2C(Cc2ncccc2Br)N1Cc1ccc2[nH]cnc2c1]

The title compound 268 was prepared according to general procedure F as a white solid (10 mg, 36.9%). MS (ESI) m/z 433 [M+H]⁺. ¹H NMR (400 MHz, DMSO-d₆) δ 13.17 (s, 1H), 8.58 (dd, J=4.6, 1.6 Hz, 1H), 8.19 (s, 1H), 8.02 (dd, J=8.0, 1.8 Hz, 1H), 7.79-7.73 (m, 1H), 7.56-7.27 (m, 3H), 7.26-7.20 (m, 2H), 6.98 (q, J=3.2 Hz, 2H), 5.18 (d, J=15.2 Hz, 1H), 5.11 (t, J=6.8 Hz, 1H), 4.45 (d, J=15.2 Hz, 1H), 3.61 (dd, J=14.8, 6.0 Hz, 1H), 3.18 (dd, J=14.8, 7.2 Hz, 1H).

### 2-((3-amino-[1,2,4]triazolo[4,3-a]pyridin-7-yl)methyl)-3-((3-bromopyridin-2-yl)methyl) isoindolin-1-one (269)

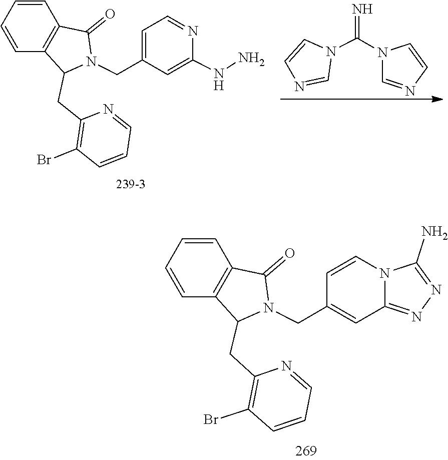 [SMILES: NNc1cc(CN2C(=O)c3ccccc3C2Cc2ncccc2Br)ccn1 ; N=C(n1ccnc1)n1ccnc1 ; Nc1nnc2cc(CN3C(=O)c4ccccc4C3Cc3ncccc3Br)ccn12]

To a solution of 3-((3-bromopyridin-2-yl)methyl)-2-((2-hydrazineylpyridin-4-yl)methyl)isoindolin-1-one (100 mg, 0.24 mmol) in DMF (3 ml), di(imidazol-1-yl)methanimine (38 mg, 0.24 mmol) was added under N₂. After addition, the mixture was stirred at 70° C. for 0.5 h. LC-MS showed the starting material was mostly converted to the product. The reaction mixture was concentrated and purified by prep HPLC to give 2-((3-amino-[1,2,4]triazolo[4,3-a]pyridin-7-yl)methyl)-3-((3-bromopyridin-2-yl)methyl) isoindolin-1-one (20 mg, 15%) as a white solid. MS (ESI) m/z 449 [M+H]⁺. ¹H NMR (400 MHz, DMSO-d₆) δ 8.56-8.49 (m, 1H), 8.45-8.43 (m, 11H), 8.01-7.98 (m, 2H), 7.79-7.72 (m, 1H), 7.59-7.45 (m, 2H), 7.29-7.24 (m, 1H), 7.10-7.05 (m, 2H), 6.59-6.51 (m, 2H), 5.30-5.23 (m, 1H), 5.00-4.90 (m, 1H), 4.57-4.46 (m, 1H), 3.63-3.47 (m, 1H), 3.30-3.15 (m, 1H).

### 2-((1-((3-bromopyridin-2-yl)methyl)-3-oxoisoindolin-2-yl)methyl)-5-oxa-7-azaspiro[3.4]octan-6-one (270)

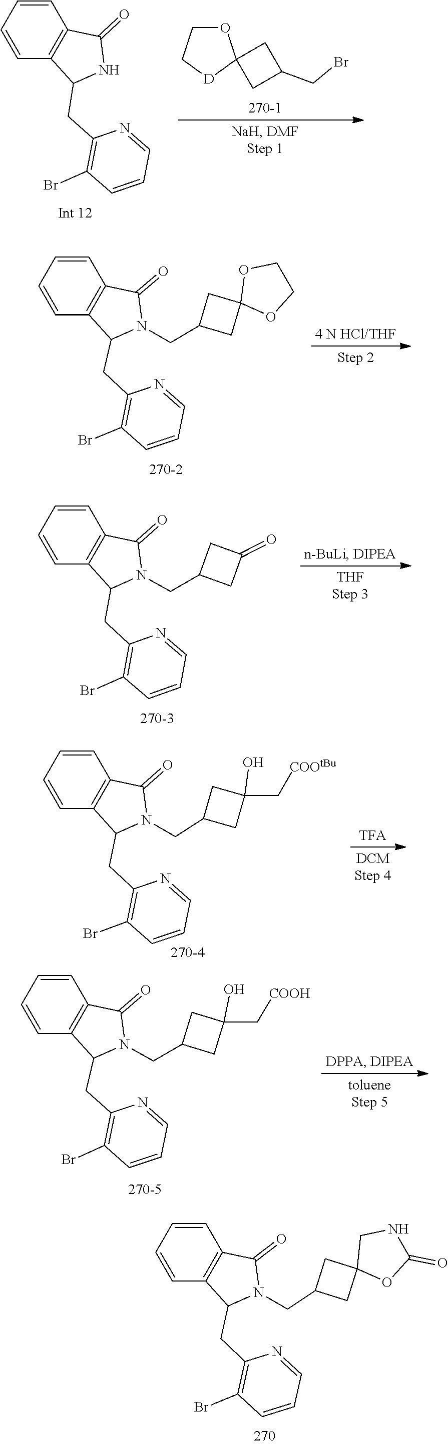

### Step 1. 3-[(3-bromo-2-pyridyl)methyl]-2-(5,8-dioxaspiro[3.4]octan-2-ylmethyl) isoindolin-1-one (270-2)

To a solution of 3-[(3-bromo-2-pyridyl) methyl]isoindolin-1-one (600 mg, 1.98 mmol) in DMF (2 mL), was added NaH (95 mg, 2.38 mmol) at 0° C. under N₂. After addition, the mixture was stirred at 0° C. for 1 h. Then the reaction mixture was added 2-(bromomethyl)-5,8-dioxaspiro[3.4]octane (410 mg, 1.98 mmol) in DMF (1 mL) at 0° C. under N₂. After addition, the mixture was stirred at 75° C. overnight. LC-MS showed the starting material was mostly converted to the product. Then the reaction mixture was added water (10 mL) and extracted with EtOAc (10 mL×3). The organics were then combined and dried (Na₂SO₄) before concentration to dryness. The residue was purified with flash (PE/EtOAc=1:1) to give the product 3-[(3-bromo-2-pyridyl)methyl]-2-(5,8-dioxaspiro[3.4]octan-2-ylmethyl)isoindolin-1-one (723 mg, 85.1%) as a yellow oil. MS (ESI) m/z 429 [M+H]⁺.

### Step 2. 3-[(3-bromo-2-pyridyl)methyl]-2-[(3-oxocyclobutyl)methyl]isoindolin-1-one (270-3)

To a solution of 3-[(3-bromo-2-pyridyl)methyl]-2-(5,8-dioxaspiro[3.4]octan-2-ylmethyl) isoindolin-1-one (723 mg, 1.68 mmol) in THF (5 ml) was added 4N HCl (5 mL) and reacted at 50° C. for 2h. LC-MS showed the starting material was mostly converted to the product. Then the reaction mixture was added sat. NaHCO₃ (20 mL) and extracted with EtOAc (10 mL×3). The organics were combined and dried (Na₂SO₄) before concentration to dryness. The crude was purified by flash (PE/EtOAc=1:1) to give the product 3-[(3-bromo-2-pyridyl)methyl]-2-[(3-oxocyclobutyl)methyl]isoindolin-1-one (500 mg, 77.1%) as a yellow oil. MS (ESI) m/z 385 [M+H]⁺.

### Step 3. tert-butyl 2-[3-[[1-[(3-bromo-2-pyridyl)methyl]-3-oxo-isoindolin-2-yl]methyl]-1-hydroxy-cyclobutyl]acetate (270-4)

To a solution of diisopropylamine (0.14 mL, 0.97 mmol) in THF (2 mL), was added n-BuLi (0.39 mL, 0.93 mmol) at −78° C. and stirred for 0.5 h under N₂. Then the mixture was added dropwise tert-butyl acetate (0.12 mL, 0.88 mmol) at −78° C. under N₂, and the mixture was stirred for 1 h, then followed by addition of 3-[(3-bromo-2-pyridyl)methyl]-2-[(3-oxocyclobutyl) methyl]isoindolin-1-one (170 mg, 0.44 mmol) in THE (2 mL) and stirred for 1 h at −78° C. LC-MS showed the starting material was mostly converted to the product. Then the reaction mixture was added water (20 mL) and extracted with EtOAc (20 mL×3). The organics were then combined and dried (Na₂SO₄) before concentration to dryness. The crude was purified with flash (PE/EtOAc=1:2) to give tert-butyl 2-[3-[[1-[(3-bromo-2-pyridyl)methyl]-3-oxo-isoindolin-2-yl]methyl]-1-hydroxy-cyclobutyl]acetate (150 mg, 67.8%) as a yellow oil. MS (ESI) m/z 487 [M+H]⁺.

### Step 4. 2-[3-[[1-[(3-bromo-2-pyridyl)methyl]-3-oxo-isoindolin-2-yl]methyl]-1-hydroxy-cyclobutyl]acetic acid (270-5)

To a solution of tert-butyl 2-[3-[[1-[(3-bromo-2-pyridyl)methyl]-3-oxo-isoindolin-2-yl]methyl]-1-hydroxy-cyclobutyl]acetate (641 mg, 1.28 mmol) in DCM (6 mL) was added TFA (6 mL) at 0° C., then up to r.t. and stirred for 1 h. LC-MS showed the starting material was mostly converted to the product. The mixture was concentrated under reduced pressure. The crude product 2-[3-[[1-[(3-bromo-2-pyridyl)methyl]-3-oxo-isoindolin-2-yl]methyl]-1-hydroxy-cyclobutyl]acetic acid (574 mg) was used in the next step without purification. MS (ESI) m/z 431 [M+H]⁺.

### Step 5. 2-[[1-[(3-bromo-2-pyridyl)methyl]-3-oxo-isoindolin-2-yl]methyl]-5-oxa-7-azaspiro[3.4]octan-6-one (270)

To a solution of 2-[3-[[1-[(3-bromo-2-pyridyl)methyl]-3-oxo-isoindolin-2-yl]methyl]-1-hydroxy-cyclobutyl]acetic acid (574 mg, 1.29 mmol) in toluene (15 mL) were added DIPEA (1.1 mL, 6.44 mmol) and DPPA (0.56 mL, 2.58 mmol) at 25° C., then up to 120° C. and stirred overnight. LC-MS showed the starting material was mostly converted to the product. The mixture was concentrated under reduced pressure. The crude was purified with flash (PE/EtOAc=3:1) to give the product 2-[[1-[(3-bromo-2-pyridyl)methyl]-3-oxo-isoindolin-2-yl]methyl]-5-oxa-7-azaspiro[3.4]octan-6-one (423 mg, 74.2%) as a white solid. MS (ESI) m/z 442 [M+H]⁺. ¹H NMR (400 MHz, CDCl₃) δ 8.59 (d, J=4.7 Hz, 1H), 7.91 (d, J=8.0 Hz, 1H), 7.87-7.79 (m, 1H), 7.42 (m, 2H), 7.17 (dd, J=8.1, 4.7 Hz, 1H), 6.96 (d, J=6.9 Hz, 1H), 5.60 (s, 1H), 5.30 (t, J=6.7 Hz, 1H), 4.10 (dd, J=14.5, 3.0 Hz, 1H), 3.54 (m, 3H), 3.21 (dt, J=13.0, 6.2 Hz, 2H), 2.31 (m, 5H).

### 3-((3-bromopyridin-2-yl)methyl)-2-((6-fluoro-1H-benzo[d][1,2,3]triazol-5-yl)methyl) isoindolin-1-one (271)

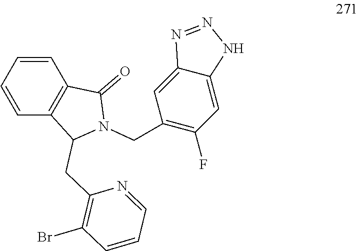 [SMILES: O=C1c2ccccc2C(Cc2ncccc2Br)N1Cc1cc2nn[nH]c2cc1F]

The title compound 271 was prepared according to general procedure F using 5-(bromomethyl)-6-fluoro-1-((2-(trimethylsilyl)ethoxy)methyl)-1H-benzo[d][1,2,3]triazole (preparation according to the procedure outlined for Int 4) as a starting material to afford a white solid (20 mg, 24.8%). MS (ESI) m/z 452 [M+H]⁺. ¹H NMR (400 MHz, CDCl₃) δ 8.54 (d, J=4.6 Hz, 1H), 7.95-7.89 (m, 1H), 7.81 (d, J=8.1 Hz, 1H), 7.51-7.47 (m, 2H), 7.33 (s, 1H), 7.18-7.12 (m, 2H), 6.79 (s, 1H), 5.36 (t, J=6.5 Hz, 1H), 5.19 (d, J=15.6 Hz, 1H), 4.60 (d, J=15.7 Hz, 1H), 3.52 (dd, J=15.3, 6.8 Hz, 1H), 3.32 (dd, J=15.3, 5.9 Hz, 1H).

### 4-chloro-3-((7-methyl-3-oxo-2-((2-oxo-2,3-dihydrobenzo[d]oxazol-6-yl)methyl)isoindolin-1-yl)methyl)picolinonitrile (272)

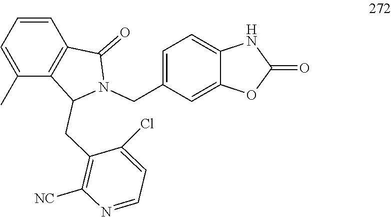 [SMILES: Cc1cccc2c1C(Cc1c(Cl)ccnc1C#N)N(Cc1ccc3[nH]c(=O)oc3c1)C2=O]

The title compound 272 was prepared according to the procedure described for compound 212 as a white solid (5 mg, 30%). MS (ESI) m/z 445 [M+H]⁺. ¹H NMR (400 MHz, DMSO-d₆) δ 11.63 (s, 1H), 8.68 (d, J=5.2 Hz, 1H), 8.00 (d, J=5.2 Hz, 1H), 7.63 (d, J=7.2 Hz, 1H), 7.48 (t, J=7.2 Hz, 1H), 7.42 (d, J=7.2 Hz, 1H), 6.97 (d, J=8.0 Hz, 1H), 6.65 (d, J=8.0 Hz, 1H), 5.12 (d, J=15.6 Hz, 11H), 4.99 (dd, J=8.0, 5.6 Hz, 1H), 3.76 (d, J=15.6 Hz, 1H), 3.49 (dd, J=14.0, 5.2 Hz, 1H), 3.27-3.23 (m, 1H), 2.09 (s, 3H).

### rel-(S)-6-((5-((dimethylamino)methyl)-3-methyl-1-oxoisoindolin-2-yl)methyl)benzo[d]oxazol-2(3H)-one (273)

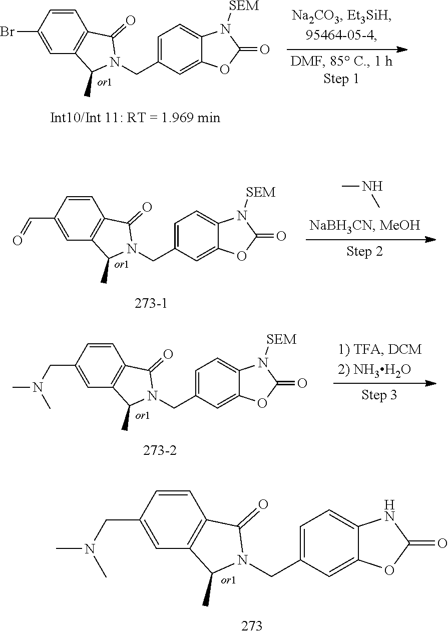

### Step 1: rel-(S)-3-methyl-1-oxo-2-((2-oxo-3-((2-(trimethylsilyl)ethoxy)methyl)-2,3-dihydrobenzo[d]oxazol-6-yl)methyl)isoindoline-5-carbaldehyde (273-1)

To a solution of the stereoisomer of Int 10 or Int 11 that has RT of 1.969 min (500 mg, 1 mmol) in DMF (20 ml), Pd(dppf)Cl₂ (72 mg, 0.1 mmol), Et₃SiH (229 mg, 2 mmol) and Na₂CO₃ (206 mg 2 mmol) were added under N₂. The reaction mixture was stirred at 85° C. for 1 h. The resulting mixture was diluted with EtOAc (30 mL), the organic phase was successively washed with water (30 mL) and brine (30 mL), dried over anhydrous Na₂SO₄, after filtration, the filtrate was concentrated under reduced pressure. The residue was purified by TLC, Layered with (PE:EtOAc) (1:1) to afford the title compound (440 mg, 97%) as a white solid. MS (ESI) m/z 453 [M+H]⁺.

### Step 2: rel-(S)-6-((5-((dimethylamino)methyl)-3-methyl-1-oxoisoindolin-2-yl)methyl)-3-((2-(trimethylsilyl)ethoxy)methyl)benzo[d]oxazol-2(3H)-one (273-2)

273-1 (130 mg, 0.3 mmol) and dimethylamine (64 mg 1.4 mmol) were dissolved in MeOH (10 ml). The reaction mixture was stirred at 25° C. for 0.5h. NaBH₃CN (90 mg, 1.4 mmol) was added in reaction mixture. The reaction mixture was stirred at 25° C. for 2h. H₂O (10 mL) was added in mixture. The resulting mixture was diluted with EtOAc (30 mL), the organic phase was successively washed with water (30 mL) and brine (30 mL), dried over anhydrous Na₂SO₄, after filtration, the filtrate was concentrated under reduced pressure. The residue was purified by TLC, layered with (EtOAc) to afford the title compound (66 mg, 42%) as a white solid. MS (ESI) m/z 482 [M+H]⁺.

### Step 3: rel-(S)-6-((5-((dimethylamino)methyl)-3-methyl-1-oxoisoindolin-2-yl) methyl)benzo[d]oxazol-2(3H)-one (273)

To a solution of 273-2 (66 mg, 0.1 mmol) in DCM (5 mL) was added dropwise TFA (5 mL) at 0° C. The resulting mixture was stirred for additional 0.5 h at 0° C. LC-MS and TLC showed 273-2 disappeared. The resulting mixture was concentrated under reduced pressure. NH₄OH (5 mL) was dropwise added in mixture under 0° C. The resulting mixture was stirred for additional 0.5 h at 0° C. The mixture was concentrated under reduced pressure. The residue was purified by TLC, layered with (DCM: MeOH) (50:1) to afford the title compound (25 mg, 43%) as a white solid. MS (ESI) m/z 352 [M+H]⁺. ¹H NMR (400 MHz, CD₃OD) δ 7.90 (d, J=7.8 Hz, 1H), 7.74 (s, 1H), 7.67 (d, J=7.8 Hz, 1H), 7.22 (s, 1H), 7.19 (d, J=8.0 Hz, 1H), 7.07 (d, J=8.0 Hz, 1H), 5.17 (d, J=15.2 Hz, 1H), 4.58 (d, J=6.7 Hz, 1H), 4.52 (d, J=15.2 Hz, 1H), 4.38 (s, 1H), 2.82 (s, 6H), 1.50 (d, J=6.8 Hz, 3H).

### rel-(S)-6-((5-(azetidin-1-ylmethyl)-3-methyl-1-oxoisoindolin-2-yl)methyl)benzo[d]oxazol-2(3H)-one (274)

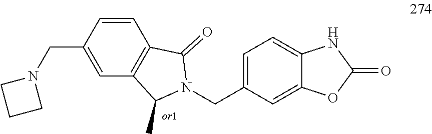 [SMILES: C[C@H]1c2cc(CN3CCC3)ccc2C(=O)N1Cc1ccc2[nH]c(=O)oc2c1]

The title compound 274 was prepared according to the procedure described for compound 273 using the stereoisomer of Int 10 or Int 11 that has RT of 1.969 min as a starting material to afford a white solid (19 mg, 52%). MS (ESI) m/z 364 [M+H]⁺. ¹H NMR (400 MHz, CD₃OD) δ 7.87 (d, J=7.8 Hz, 11H), 7.67 (s, 1H), 7.62 (d, J=7.8 Hz, 11H), 7.20 (s, 11H), 7.17 (d, J=8.1 Hz, 1H), 7.07 (d, J=8.0 Hz, 11H), 5.16 (d, J=15.2 Hz, 11H), 4.55 (d, J=6.7 Hz, 11H), 4.50 (d, J=15.2 Hz, 1H), 4.46 (s, 2H), 4.14 (t, J=8.2 Hz, 4H), 2.53-2.46 (m, 2H), 1.48 (d, J=6.7 Hz, 3H).

### 6-((5-fluoro-3-methyl-1-oxoisoindolin-2-yl)methyl)benzo[d]oxazol-2(3H)-one (275)

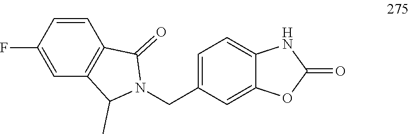 [SMILES: CC1c2cc(F)ccc2C(=O)N1Cc1ccc2[nH]c(=O)oc2c1]

The title compound 275 was prepared according to the procedure described for compound 48 to afford a white solid (4 mg, 22%). MS (ESI) m/z 313 [M+H]⁺. ¹H NMR (400 MHz, CDCl₃) δ 7.89-7.84 (m, 11H), 7.76 (s, 1H), 7.20-7.14 (m, 2H), 7.11 (d, J=7.9 Hz, 1H), 7.08-7.03 (m, 1H), 6.98 (d, J=8.0 Hz, 1H), 5.26 (d, J=15.2 Hz, 1H), 4.44-4.24 (m, 2H), 1.44 (d, J=6.8 Hz, 3H).

### 3-((4-chloro-2-cyanopyridin-3-yl)methyl)-1-oxo-2-((2-oxo-2,3-dihydrobenzo[d]oxazol-6-yl)methyl)isoindoline-5-carbonitrile (276)

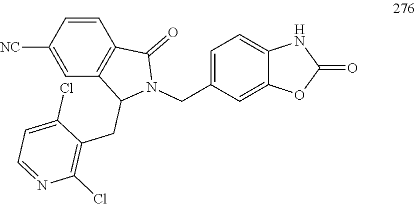 [SMILES: N#Cc1ccc2c(c1)C(Cc1c(Cl)ccnc1Cl)N(Cc1ccc3[nH]c(=O)oc3c1)C2=O]

The title compound 276 was prepared according to the procedure described for compound 212 using 1-oxo-2-((2-oxo-3-((2-(trimethylsilyl)ethoxy)methyl)-2,3-dihydrobenzo[d]oxazol-6-yl)methyl)isoindoline-5-carbonitrile as a starting material to afford a white solid (10 mg, 30%). MS (ESI) m/z 456 [M+H]⁺. ¹H NMR (400 MHz, DMSO-d₆) δ 11.65 (s, 1H), 8.70 (d, J=5.2 Hz, LH), 8.04-7.96 (m, 3H), 7.25 (s, 1H), 7.20 (s, LH), 7.04 (s, 2H), 5.19 (d, J=15.6 Hz, 1H), 4.89 (dd, J=9.2, 5.6 Hz, 1H), 4.49 (d, J=15.6 Hz, 1H), 3.82 (dd, J=14.0, 5.6 Hz, 1H), 2.94 (dd, J=14.0, 9.2 Hz, 1H).

### 6-((1-((4-chloro-1-(2-methoxyethyl)-1H-pyrazol-3-yl)methyl)-3-oxoisoindolin-2-yl)methyl) benzo[d]oxazol-2(3H)-one (277)

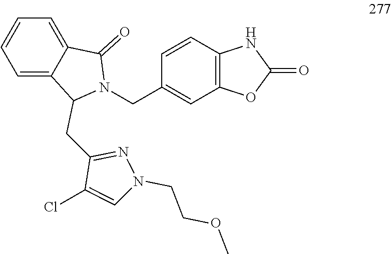 [SMILES: COCCn1cc(Cl)c(CC2c3ccccc3C(=O)N2Cc2ccc3[nH]c(=O)oc3c2)n1]

The title compound 277 was prepared according to general procedure E as a white solid (37 mg, 16%). MS (ESI) m/z 453 [M+H]⁺. ¹H NMR (400 MHz, DMSO-d₆) δ 11.67 (bs, 1H), 7.75-7.72 (m, 1H), 7.62 (s, 1H), 7.52-7.47 (m, 2H), 7.16 (s, 1H), 7.06-7.00 (m, 2H), 6.77-6.74 (m, 1H), 5.20 (d, J=15.2 Hz, 1H), 4.70 (dd, J=8.8, 5.6 Hz, 1H), 4.34 (d, J=15.2 Hz, 1H), 4.06-3.94 (m, 2H), 3.58-3.44 (m, 3H), 3.05 (s, 3H), 2.85 (dd, J=14.8, 5.2 Hz, 1H).

The intermediate 277-3 was prepared as follows:

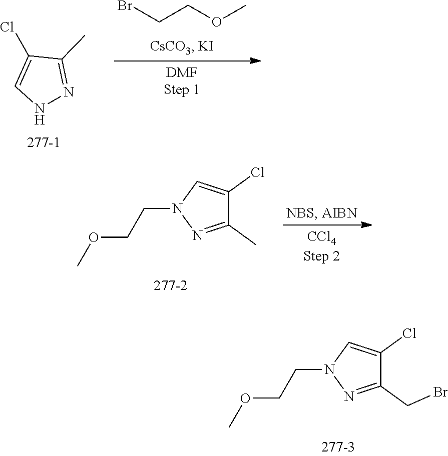

### Step 1. 4-chloro-1-(2-methoxyethyl)-3-methyl-1H-pyrazole (277-2)

To a solution of 4-chloro-3-methyl-1H-pyrazole (300 mg, 2.58 mmol) in DMF (10 mL) were added Cs₂CO₃ (1.68 g, 5.15 mmol), 1-bromo-2-methoxyethane (394 mg, 2.83 mmol) and KI (43 mg, 0.26 mmol) and stirred for 4 h at 70° C. The mixture was diluted with EA (30 mL) and washed with water (3×20 mL). The organic layer was separated, dried over anhydrous Na₂SO₄ and concentrated under vacuum. The residue was purified by silica gel flash column chromatography (PE/EA=4/1) to afford the title compound (300 mg, 66.8%) as a yellow oil. MS (ESI) m/z 175 [M+H]⁺.

### Step 2. 3-(bromomethyl)-4-chloro-1-(2-methoxyethyl)-1H-pyrazole (277-3)

To a solution of 4-chloro-1-(2-methoxyethyl)-3-methyl-1H-pyrazole (300 mg, 1.72 mmol) in CCl₄ (5 mL) were added NBS (307 mg, 1.72 mmol) and AIBN (28 mg, 0.17 mmol). The mixture was refluxed for 5 h. Then the reaction mixture was quenched with water and extracted with EA (2×30 mL) and concentrated. The residue was purified by silica gel flash column chromatography (PE/EA=5/1) to afford the title compound (130 mg, 30%) as a yellow oil. MS (ESI) m/z 253/255 [M+H]⁺.

### 4-((2-((1H-benzo[d][1,2,3]triazol-5-yl)methyl)-3-oxoisoindolin-1-yl)methyl)-5-chloronicotinonitrile (278)

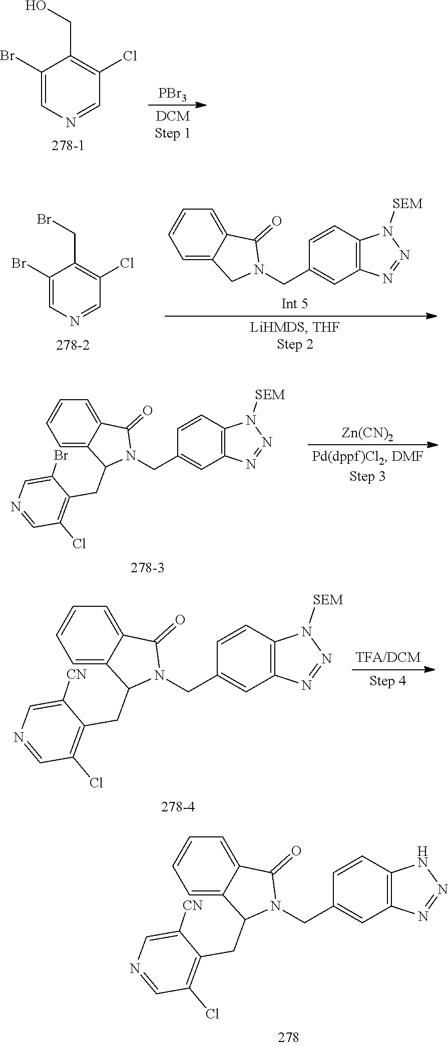

### Step 1. 3-bromo-4-(bromomethyl)-5-chloropyridine (278-2)

To a solution of (3-bromo-5-chloropyridin-4-yl)methanol (200 mg, 0.89 mmol) in DCM (5 mL) was added PBr₃ (268 mg, 0.99 mmol) at 0° C. for 2 h. The reaction mixture was quenched with water and pH was adjusted to 7-8 by NaHCO₃(aq.) and extracted with DCM (2×30 mL) and concentrated to afford the title compound (138 mg, 54.8%) as a white solid. MS (ESI) m/z 284/286 [M+H]+

### Step 2. 3-((3-bromo-5-chloropyridin-4-yl)methyl)-2-((1-((2-(trimethylsilyl)ethoxy) methyl)-1H-benzo[d][1,2,3]triazol-5-yl)methyl)isoindolin-1-one (278-3)

To a solution of Int 5 (200 mg, 0.51 mmol) in THF (20 mL) were added LiHMDS (0.51 mL, 0.51 mmol) at −78° C. for 0.5 h. Then 3-bromo-4-(bromomethyl)-5-chloropyridine (144 mg, 0.51 mmol) was added. The resulting mixture was stirred for 1 h at −78° C. and diluted with EA (30 mL) and washed with water (3×20 mL). The organic layer was separated, dried over anhydrous Na₂SO₄ and concentrated under vacuum. The residue was purified by silica gel flash column chromatography (PE/EA=4/1) to afford the title compound (100 mg, 33.0%) as a yellow solid. MS (ESI) m/z 598 [M+H]⁺.

### Step 3. 5-chloro-4-((3-oxo-2-((1-((2-(trimethylsilyl)ethoxy)methyl)-1H-benzo[d][1,2,3]triazol-5-yl)methyl)isoindolin-1-yl)methyl)nicotinonitrile (278-4)

To a solution of 3-((3-bromo-5-chloropyridin-4-yl)methyl)-2-((1-((2-(trimethylsilyl) ethoxy)methyl)-1H-benzo[d][1,2,3]triazol-5-yl)methyl)isoindolin-1-one (100 mg, 0.17 mmol) in DMF (5 mL) were added Pd(dppf)C₁₂ (24 mg, 0.03 mmol) and Zn(CN)₂ (10 mg, 0.08 mmol). The mixture was stirred at 100° C. overnight under N₂. The reaction mixture was diluted with EA (30 mL) and washed with water (3×20 mL). The organic layer was separated, dried over anhydrous Na₂SO₄ and concentrated under vacuum. The residue was purified by TLC to afford the title compound (50 mg, 54.7%) as a yellow oil. MS (ESI) m/z 545 [M+H]⁺.

### Step 4. 4-((2-((1H-benzo[d][1,2,3]triazol-5-yl)methyl)-3-oxoisoindolin-1-yl)methyl)-5-chloronicotinonitrile (278)

To a solution of 5-chloro-4-((3-oxo-2-((1-((2-(trimethylsilyl)ethoxy)methyl)-1H-benzo[d][1,2,3]triazol-5-yl)methyl)isoindolin-1-yl)methyl)nicotinonitrile (50 mg, 0.09 mmol) in DCM (5 mL) was added trifluoroacetic acid (2 mL). The mixture was stirred at r.t. overnight, and then concentrated under vacuum. The residue was purified by Prep-HPLC to afford 6-((1-((3-methylpyridin-2-yl) methyl)-3-oxoisoindolin-2-yl) methyl) benzo[d]oxazol-2(3H)-one (2 mg, 5.3%) as a white solid. MS (ESI) m/z 415 [M+H]⁺. ¹H NMR (400 MHz, DMSO-d₆) δ 15.64 (s, 1H), 9.00-8.92 (m, 2H), 8.00 (t, J=8.0 Hz, 1H), 7.94-7.86 (m, 1H), 7.84-7.80 (m, 1H), 7.57-7.46 (m, 2H), 7.24-7.20 (m, 1H), 6.72-6.68 (m, 1H), 5.34 (d, J=15.6 Hz, 1H), 4.85-4.79 (m, 1H), 4.65 (d, J=15.6 Hz, 1H), 3.87-3.79 (m, 1H), 2.95-2.89 (m, 1H).

### 2-((1H-benzo[d][1,2,3]triazol-5-yl)methyl)-3-((4-chloro-1-methyl-1H-pyrazol-5-yl)methyl) isoindolin-1-one (279)

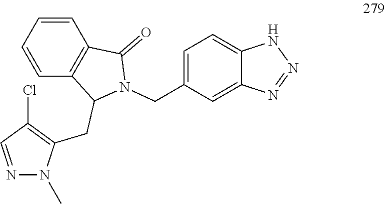 [SMILES: Cn1ncc(Cl)c1CC1c2ccccc2C(=O)N1Cc1ccc2[nH]nnc2c1]

The title compound 279 was prepared according to the procedure described for step 2 and step 4 for the preparation of compound 278 to afford a white solid (8 mg, 4.1%). MS (ESI) m/z 393 [M+H]⁺. ¹H NMR (400 MHz, DMSO-d₆) δ 15.61 (bs, 1H), 7.80-7.69 (m, 2H), 7.55-7.46 (m, 3H), 7.40 (dd, J=8.3, 7.1 Hz, 1H), 7.17 (d, J=7.0 Hz, 1H), 6.80 (dd, J=5.4, 3.2 Hz, 1H), 5.54 (d, J=15.8 Hz, 1H), 4.94 (d, J=15.8 Hz, 1H), 4.83 (dd, J=8.6, 5.6 Hz, 1H), 3.59 (dd, J=14.8, 5.6 Hz, 1H), 3.55 (s, 3H), 3.03 (dd, J=14.8, 8.6 Hz, 1H).

The intermediate 279-4 was prepared as follows:

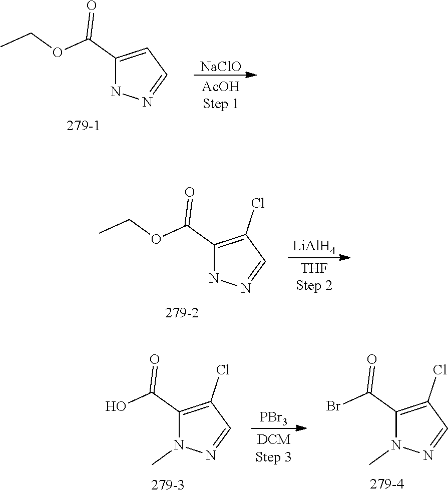

### Step 1. ethyl 4-chloro-1-methyl-1H-pyrazole-5-carboxylate (279-2)

To a solution of ethyl 1-methyl-1H-pyrazole-5-carboxylate (462 mg, 3 mmol) in AcOH (10 mL) was added NaClO (7 mL) at r.t. for 6 h. The mixture was diluted with EA (30 mL) and washed with water (3×20 mL). The organic layer was separated, dried over anhydrous Na₂SO₄ and concentrated under vacuum to afford the title compound (400 mg, 71.9%) as a yellow oil. MS (ESI) m/z 189 [M+H]⁺.

### Step 2. (4-chloro-1-methyl-1H-pyrazol-5-yl)methanol (279-3)

To a solution of ethyl 4-chloro-1-methyl-1H-pyrazole-5-carboxylate (300 mg, 1.6 mmol) in THF (5 mL) was added LiAlH₄ (610 mg, 1.6 mmol) at 0° C. for 1 h. The reaction mixture was quenched with water and extracted with EA (2×30 mL) and concentrated to afford the title compound (180 mg, 77%) as a light yellow oil. MS (ESI) m/z 147 [M+H]⁺.

### Step 3. 5-(bromomethyl)-4-chloro-1-methyl-1H-pyrazole (279-4)

To a solution of (4-chloro-1-methyl-1H-pyrazol-5-yl)methanol (180 mg, 1.23 mmol) in DCM (5 mL) was added PBr₃ (354 mg, 1.35 mmol) at 0° C. for 2 h. The reaction mixture was quenched with water and the pH was adjusted to 7-8 by NaHCO₃(aq.) Then extracted with DCM (2×30 mL) and concentrated to afford the title compound (200 mg, 78.1%) as a white solid. MS (ESI) m/z 209/211 [M+H]⁺.

### 2-((1-((3-bromopyridin-2-yl)methyl)-3-oxoisoindolin-2-yl)methyl)thiazole-5-carboxamide (280)

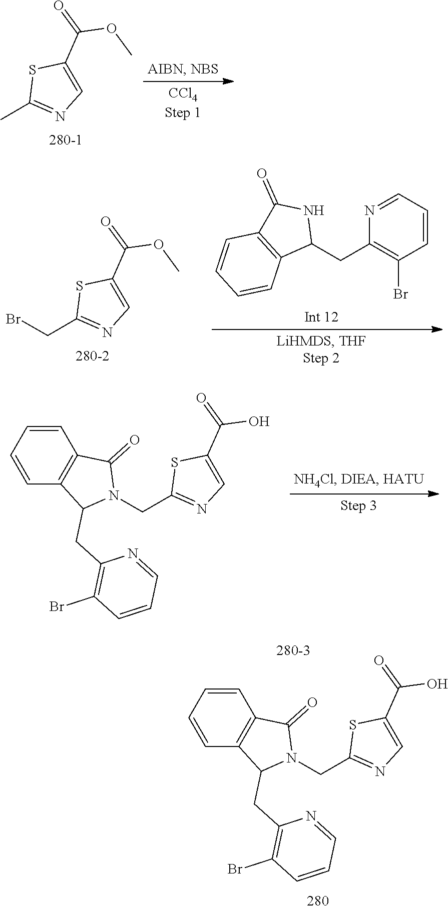

### Step 1. methyl 2-(bromomethyl)thiazole-5-carboxylate (280-2)

To a solution of methyl 2-methylthiazole-5-carboxylate (1 g, 6.36 mmol) in CCl₄ (100 mL) were added NBS (1.2 g, 7 mmol) and AIBN (103 mg, 0.63 mmol) at r.t. The reaction was refluxed for 3 h. H₂O (50 mL) was added, and the mixture was extracted with EA (50 mL*2). The combined organic layers were washed with H₂O (100 mL*2) and brine (50 mL), dried over Na₂SO₄, filtered and concentrated. The residue was purified by flash column chromatography (PE/EA=5/1) to give methyl 2-(bromomethyl)thiazole-5-carboxylate (450 mg, 30%) as yellow oil. MS (ESI) m/z 236 [M+H]H⁺.

### Step 2. 2-((1-((3-bromopyridin-2-yl)methyl)-3-oxoisoindolin-2-yl)methyl)thiazole-5-carboxylic acid (280-3)

To a solution of 3-((3-bromopyridin-2-yl)methyl)isoindolin-1-one (325 mg, 1.06 mmol) in THF (10 mL) was added LiHMDS (1.59 ml, 1.0 M in THF, 1.59 mmol) at −40° C. The solution was stirred at −40° C. for 0.5 h. Then a solution of methyl 2-(bromomethyl)thiazole-5-carboxylate (300 mg, 1.27 mmol) in THF (2 mL) was added at −40° C. The mixture was stirred at 50° C. for 3 h. Sat. NH₄Cl (10 mL) was added at r.t., and the mixture was extracted with EA (50 mL*2). The pH of the aqueous phase was adjusted to 7 with 0.1N HCl, and the reaction mixture was extracted with EA (50 mL*2). The combined organic layers were washed with H₂O (100 mL*2) and brine (50 mL), dried over Na₂SO₄, filtered and concentrated. The residue was purified by FCC to give 2-((1-((3-bromopyridin-2-yl)methyl)-3-oxoisoindolin-2-yl)methyl)thiazole-5-carboxylic acid (60 mg, 13%) as a yellow solid. MS (ESI) m/z 444 [M+H]⁺.

### Step 3. 2-((1-((3-bromopyridin-2-yl)methyl)-3-oxoisoindolin-2-yl)methyl)thiazole-5-carboxamide (280)

To a solution of 2-((1-((3-bromopyridin-2-yl)methyl)-3-oxoisoindolin-2-yl)methyl) thiazole-5-carboxylic acid (60 mg, 0.13 mmol) in DMF (5 mL) were added NH₄Cl (37 mg, 0.67 mmol), DIEA (52 mg, 0.4 mmol) and HATU (62 mg, 0.67 mmol) at ice-bath. The reaction was stirred at r.t. overnight. H₂O (50 mL) was added, and the mixture was extracted with EA (50 mL*2). The combined organic layers were washed with H₂O (50 mL*2) and brine (50 mL), dried over Na₂SO₄, filtered and concentrated. Then the residue was purified by prep-HPLC to give 2-((1-((3-bromopyridin-2-yl)methyl)-3-oxoisoindolin-2-yl)methyl)thiazole-5-carboxamide (5 mg, 8%) as a white solid. MS (ESI) m/z 443 [M+H]⁺. ¹H NMR (300 MHz, DMSO-d₆) δ 8.57-8.55 (m, 1H), 8.20 (s, 1H), 8.08-8.05 (m, 2H), 7.78-7.76 (m, 1H), 7.61-7.54 (m, 3H), 7.30 (dd, J=4.8, 8.1 Hz, 1H), 7.21 (d, J=7.2 Hz 11H), 5.48 (t, J=6.3 Hz, 1H), 5.22 (d, J=16.8 Hz, 11H), 4.86 (d, J=16.8 Hz, 1H), 3.64 (dd, J=15.6, 6.0 Hz, 1H), 3.31-3.30 (m, 1H). 7-((1-((3-bromopyridin-2-yl)methyl)-3-oxoisoindolin-2-yl)methyl)-[1,2,4]triazolo[4,3-a]pyridin-3(2H)-one (281)

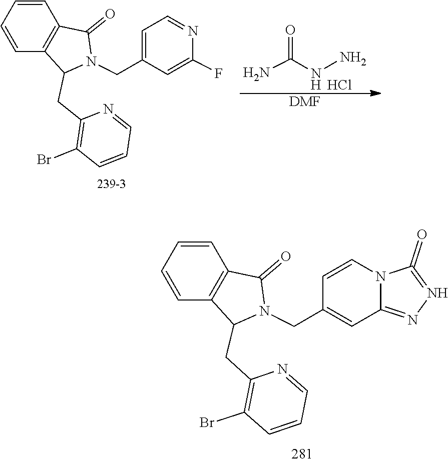

To a solution of 3-((3-bromopyridin-2-yl)methyl)-2-((2-fluoropyridin-4-yl)methyl)isoindolin-1-one (100 mg, 0.24 mmol) in DMF (3 ml) was added semicarbazide hydrochloride (27 mg, 0.24 mmol) under N₂. After addition, the mixture was stirred at 150° C. with microwave for 0.5 h. LC-MS showed the starting material was mostly converted to the product. The reaction mixture was concentrated and purified by prep-HPLC to give 7-((1-((3-bromopyridin-2-yl)methyl)-3-oxoisoindolin-2-yl)methyl)-[1,2,4]triazolo[4,3-a]pyridin-3(2H)-one (19 mg, 13.9%) as a white solid. MS (ESI) m/z 450 [M+H]⁺. ¹H NMR (400 MHz, DMSO-d₆) S 12.39 (s, 1H), 8.54 (dd, J=4.8, 1.6 Hz, 1H), 8.00 (dd, J=8.0, 1.6 Hz, 1H), 7.79-7.69 (m, 2H), 7.58-7.47 (m, 2H), 7.27 (dd, J=8.0, 4.8 Hz, 1H), 7.15 (d, J=6.6 Hz, 1H), 6.76 (s, 1H), 6.30 (dd, J=7.2, 1.6 Hz, 1H), 5.30 (t, J=6.4 Hz, 1H), 4.80 (d, J=16.4 Hz, 1H), 4.47 (d, J=16.4 Hz, 1H), 3.62-3.56 (m, 1H), 3.28 (dd, J=15.6, 6.8 Hz, 11H).

### 3-((2-((1H-benzo[d][1,2,3]triazol-5-yl)methyl)-3-oxoisoindolin-1-yl)methyl)-4-bromopicolinonitrile (282)

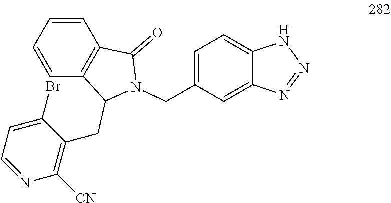 [SMILES: N#Cc1nccc(Br)c1CC1c2ccccc2C(=O)N1Cc1ccc2[nH]nnc2c1]

The title compound 282 was prepared according to the procedure described for compound 279 using 4-bromo-3-methylpicolinonitrile as a starting material to afford a white solid (5 mg, 6.4%). MS (ESI) m/z 459 [M+H]⁺. ¹H NMR (400 MHz, DMSO-d₆) δ 15.51 (bs, 1H), 8.55 (d, J=5.2 Hz, 1H), 8.12 (d, J=5.2 Hz, 1H), 7.87 (d, J=8.4 Hz, 1H), 7.83 (d, J=7.6 Hz, 1H), 7.72 (s, 1H), 7.55 (t, J=7.6 Hz, 1H), 7.48 (t, J=7.6 Hz, 1H), 7.28 (d, J=8.4 Hz, 1H), 6.55 (d, J=7.6 Hz, 1H), 5.33 (d, J=15.6 Hz, 1H), 4.86 (dd, J=9.2, 6.0 Hz, 1H), 4.62 (d, J=15.6 Hz, 1H), 3.84 (dd, J=14.0, 6.0 Hz, 1H), 2.92 (dd, J=14.0, 9.2 Hz, 1H).

### 4-((3-oxo-2-((2-oxo-2,3-dihydrobenzo[d]oxazol-6-yl)methyl)isoindolin-1-yl)methyl) pyridine-3,5-dicarbonitrile (283)

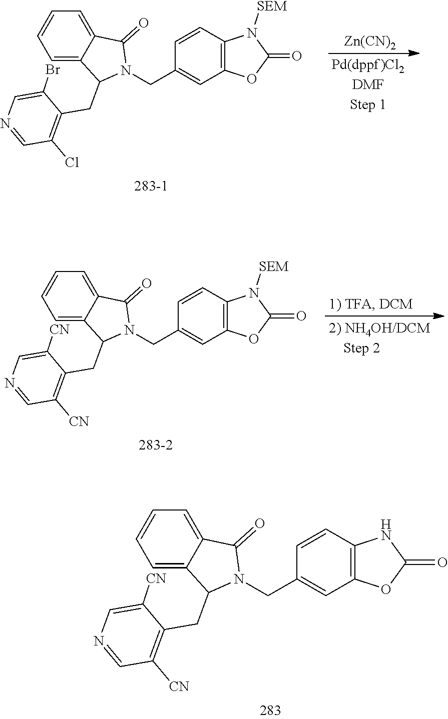

283-1 was prepared according to general procedure E using 278-2 as a starting material.

### Step 1. 4-((3-oxo-2-((2-oxo-3-((2-(trimethylsilyl)ethoxy)methyl)-2,3-dihydrobenzo[d]oxazol-6-yl)methyl)isoindolin-1-yl)methyl)pyridine-3,5-dicarbonitrile (283-2)

To a solution of 6-((1-((3-bromo-5-chloropyridin-4-yl)methyl)-3-oxoisoindolin-2-yl)methyl)-3-((2-(trimethylsilyl)ethoxy)methyl)benzo[d]oxazol-2(3H)-one (100 mg, 0.16 mmol) in DMF (5 mL) were added Pd(dppf)Cl₂ (24 mg, 0.03 mmol), Zn(CN)₂ (19 mg, 0.16 mmol) at 110° C. overnight. The reaction mixture was diluted with EA (30 mL) and washed with water (3×20 mL). The organic layer was separated, dried over anhydrous Na₂SO₄ and concentrated under vacuum. The residue was purified by TLC to afford the title compound (50 mg, 55.7%) as a yellow oil. MS (ESI) m/z 552 [M+H]⁺.

### Step 2. 4-((3-oxo-2-((2-oxo-2,3-dihydrobenzo[d]oxazol-6-yl)methyl)isoindolin-1-yl)methyl) pyridine-3,5-dicarbonitrile (283)

To a solution of 4-((3-oxo-2-((2-oxo-3-((2-(trimethylsilyl)ethoxy)methyl)-2,3-dihydrobenzo[d]oxazol-6-yl)methyl)isoindolin-1-yl)methyl)pyridine-3,5-dicarbonitrile (50 mg, 0.09 mmol) in DCM (5 mL) was added trifluoroacetic acid (2 mL). The mixture was stirred at r.t. for 2h, and then concentrated under vacuum. The residue was dissolved with DCM (2 mL), and NH₄OH (1 mL) was added dropwise. The mixture was stirred at r.t. for 1 h. The reaction mixture was concentrated, and the residue was purified by Prep-HPLC to 4-((3-oxo-2-((2-oxo-2,3-dihydrobenzo[d]oxazol-6-yl)methyl)isoindolin-1-yl)methyl)pyridine-3,5-dicarbonitrile (14 mg, 32%) as a white solid. MS (ESI) m/z 422 [M+H]⁺. ¹H NMR (400 MHz, DMSO-d₆) δ 11.67 (bs, 1H), 9.25 (s, 2H), 7.79-7.73 (m, 1H), 7.57-7.45 (m, 2H), 7.20 (d, J=1.3 Hz, 1H), 7.07-6.98 (m, 2H), 6.81 (d, J=7.1 Hz, 11H), 5.14 (d, J=15.4 Hz, 1H), 4.91 (dd, J=8.6, 5.6 Hz, 1H), 4.49 (d, J=15.4 Hz, 1H), 3.87 (dd, J=13.6, 5.6 Hz, 1H), 3.04 (dd, J=13.6, 8.6 Hz, 1H).

### 3-((2-((1H-benzo[d][1,2,3]triazol-5-yl)methyl)-3-oxoisoindolin-1-yl)methyl)-4-methylpicolinonitrile (284)

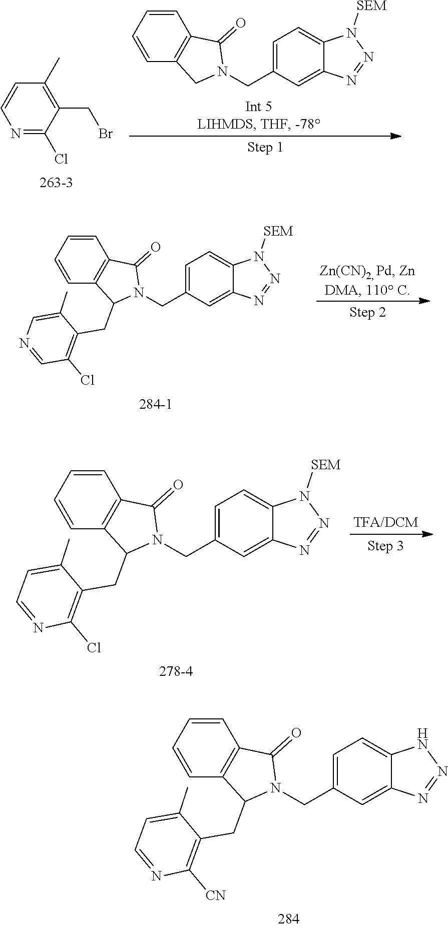

### Step 1. 3-((2-chloro-4-methylpyridin-3-yl)methyl)-2-((1-((2-(trimethylsilyl)ethoxy) methyl)-1H-benzo[d][1,2,3]triazol-5-yl)methyl)isoindolin-1-one (284-1)

To a solution of 2-((1-((2-(trimethylsilyl)ethoxy)methyl)-1H-benzo[d][1,2,3]triazol-5-yl)methyl)isoindolin-1-one (500 mg, 1.28 mmol) in dried THF (5 mL) was added dropwise LiHMDS (2 mL, 2 mmol) at −78° C. under N₂. The mixture was stirred for 40 min at −78° C., then added 3-(bromomethyl)-2-chloro-4-methylpyridine (450 mg, 2.04 mmol). The mixture was stirred for 1 h at −78° C., quenched with NH₄Cl (aq.) (2 mL), and extracted with EtOAc (2*5 mL). The combined organic layer was dried over anhydrous Na₂SO₄ and concentrated. The residue was purified by column chromatography on silica gel (eluting (Petroleum ether:EtOAc=3:1 to 1:1) to give 3-((2-chloro-4-methylpyridin-3-yl)methyl)-2-((1-((2-(trimethylsilyl) ethoxy)methyl)-1H-benzo[d][1,2,3]triazol-5-yl)methyl)isoindolin-1-one (468 mg, 69%) as a yellow oil. MS (ESI) m/z 535 [M+H]⁺.

### Step 2. 4-methyl-3-((3-oxo-2-((1-((2-(trimethylsilyl)ethoxy)methyl)-1H-benzo[d][1,2,3]triazol-5-yl)methyl)isoindolin-1-yl)methyl)picolinonitrile (284-2)

A mixture of 3-((2-chloro-4-methylpyridin-3-yl)methyl)-2-((1-((2-(trimethylsilyl) ethoxy)methyl)-1H-benzo[d][1,2,3]triazol-5-yl)methyl)isoindolin-1-one (468 mg, 0.88 mmol), zinc cyanide (308 mg, 2.62 mmol), Zn powder (30 mg, 0.4 mmol), and Pd(dppf)Cl₂ (198 mg, 0.26 mmol) in DMA (6 mL) was stirred at 110° C. under N₂ for 6 h. After cooled, the mixture was filtered, and the filtrate was concentrated. The residue was purified by column chromatography on silica gel (eluting methanol/dichloromethane 1:100) to give 4-methyl-3-((3-oxo-2-((1-((2-(trimethylsilyl)ethoxy) methyl)-1H-benzo[d][1,2,3]triazol-5-yl)methyl)isoindolin-1-yl)methyl) picolinonitrile (280 mg, 61%) as a yellow oil. MS (ESI) m/z 525 [M+H]⁺.

### Step 3. 3-((2-((1H-benzo[d][1,2,3]triazol-5-yl)methyl)-3-oxoisoindolin-1-yl)methyl)-4-methylpicolinonitrile (284)

To a solution of 4-methyl-3-((3-oxo-2-((1-((2-(trimethylsilyl)ethoxy)methyl)-1H-benzo[d][1,2,3]triazol-5-yl)methyl)isoindolin-1-yl)methyl)picolinonitrile (20 mg, 0.04 mmol) in DCM (1 mL) were added TFA (1 mL) at 0° C. The mixture was stirred for 16 h at r.t., and then concentrated. The residue was purified by Prep-HPLC to give 3-((2-((1H-benzo[d][1,2,3]triazol-5-yl)methyl)-3-oxoisoindolin-1-yl)methyl)-4-methyl picolinonitrile (6 mg, 40%) as a white solid. MS (ESI) m/z 395 [M+H]⁺. 1H NMR (400 MHz, CD₃OD) δ 8.50 (d, J=4.9 Hz, 1H), 7.90 (d, J=7.6 Hz, 1H), 7.85 (d, J=8.4 Hz, 1H), 7.79 (s, 1H), 7.55 (t, J=7.6 Hz, 1H), 7.50 (d, J=4.8 Hz, 1H), 7.45-7.39 (m, 2H), 6.49 (d, J=7.6 Hz, 1H), 5.51 (d, J=15.6 Hz, 1H), 4.85-4.81 (m, 1H), 4.64 (d, J=15.6 Hz, 1H), 3.83 (dd, J=14.0, 6.4 Hz, 1H), 2.94 (dd, J=14.0, 9.6 Hz, 1H), 2.11 (s, 3H).

### 4-chloro-3-((6-(oxetan-3-yl)-3-oxo-2-((2-oxo-2,3-dihydrobenzo[d]oxazol-6-yl)methyl) isoindolin-1-yl)methyl)picolinonitrile (285)

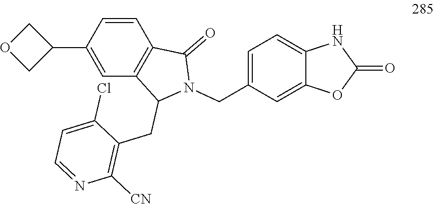 [SMILES: N#Cc1nccc(Cl)c1CC1c2cc(C3COC3)ccc2C(=O)N1Cc1ccc2[nH]c(=O)oc2c1]

The title compound 285 was prepared according to general procedure E using 6-((5-(oxetan-3-yl)-1-oxoisoindolin-2-yl)methyl)-3-((2-(trimethylsilyl)ethoxy)methyl)benzo[d]oxazol-2(3H)-one (preparation according to the procedure outlined for 198-1) as a starting material to afford a white solid (2 mg, 9.2%). MS (ESI) m/z 487 [M+H]⁺. ¹H NMR (400 MHz, CDCl₃) δ 8.61 (d, J=5.2 Hz, 1H), 7.92 (d, J=7.9 Hz, 1H), 7.80 (s, 1H), 7.62 (d, J=5.2 Hz, 1H), 7.44 (d, J=7.4 Hz, 1H), 7.13 (s, 1H), 7.10 (d, J=8.0 Hz, 1H), 6.99 (d, J=7.8 Hz, 1H), 6.62 (s, 11H), 5.37 (d, J=15.6 Hz, 1H), 5.08-5.00 (m, 2H), 4.83 (dd, J=9.2, 5.6 Hz, 1H), 4.56-4.49 (m, 2H), 4.41 (d, J=15.6 Hz, 1H), 4.16-4.12 (m, 1H), 3.75 (dd, J=13.6, 6.4 Hz, 1H), 3.05 (dd, J=13.6, 8.6 Hz, 1H).

### 4-chloro-3-((4-fluoro-3-oxo-2-((2-oxo-2,3-dihydrobenzo[d]oxazol-6-yl)methyl) isoindolin-1-yl)methyl)picolinonitrile (286)

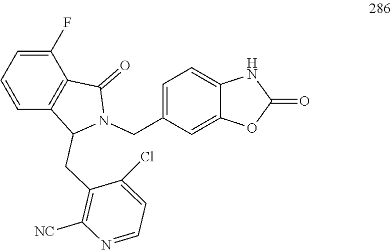 [SMILES: N#Cc1nccc(Cl)c1CC1c2cccc(F)c2C(=O)N1Cc1ccc2[nH]c(=O)oc2c1]

The title compound 286 was prepared according to the procedure described for compound 212 as a white solid (4 mg, 8%). MS (ESI) m/z 449 [M+H]-. ¹H NMR (400 MHz, DMSO-d₆) b 11.64 (bs, 1H), 8.67 (d, J=5.2 Hz, 1H), 7.98 (d, J=5.2 Hz, 1H), 7.54-7.49 (m, 1H), 7.31 (t, J=9.2 Hz, 1H), 7.21-7.19 (m, 1H), 7.06-7.03 (m, 2H), 6.41 (d, J=7.6 Hz, 1H), 5.13 (d, J=15.6 Hz, 1H), 4.83 (dd, J=9.2, 5.6 Hz, 1H), 4.43 (d, J=15.6 Hz, 1H), 3.83 (dd, J=14.0, 5.6 Hz, 1H), 2.90 (dd, J=14.0, 9.6 Hz, 1H).

### 3-((3-bromopyridin-2-yl)methyl)-2-((2-thioxo-2,3-dihydrobenzo[d]oxazol-6-yl)methyl) isoindolin-1-one (287)

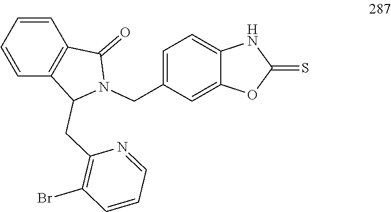 [SMILES: O=C1c2ccccc2C(Cc2ncccc2Br)N1Cc1ccc2[nH]c(=S)oc2c1]

The title compound 287 was prepared according to general procedure F using 6-methyl-3-((2-(trimethylsilyl)ethoxy)methyl)benzo[d]oxazole-2(3H)-thione as a starting material to afford a white solid (20 mg, 24.8%). MS (ESI) m/z 466 [M+H]⁺. ¹H NMR (400 MHz, DMSO-d₆) δ 8.54 (dd, J=4.8, 1.6 Hz, 1H), 8.00 (dd, J=8.0, 1.6 Hz, 1H), 7.78-7.66 (m, 1H), 7.54-7.47 (m, 2H), 7.27 (dd, J=8.0, 4.4 Hz, 1H), 7.18 (d, J=1.6 Hz, 1H), 7.12 (d, J=8.0 Hz, 1H), 7.10-7.03 (m, 1H), 7.00 (dd, J=8.0, 1.6 Hz, 1H), 5.18 (t, J=6.4 Hz, 1H), 5.04 (d, J=15.6 Hz, 1H), 4.51 (d, J=15.6 Hz, 1H), 3.56 (dd, J=15.2, 6.0 Hz, 1H), 3.22 (dd, J=15.2, 7.2 Hz, 1H).

### 3-((3-bromopyridin-2-yl)methyl)-2-((1-hydroxy-1H-benzo[d][1,2,3]triazol-5-yl)methyl) isoindolin-1-one (288)

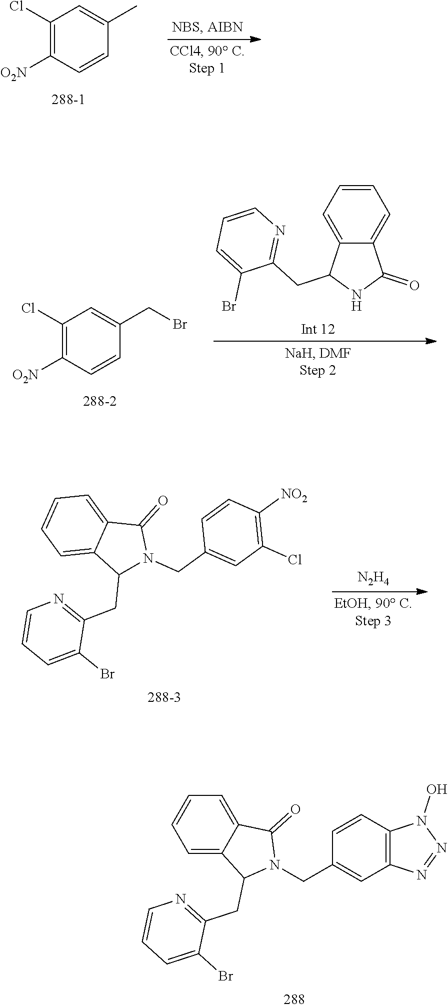 [SMILES: Cc1ccc([N+](=O)[O-])c(Cl)c1 ; O=C1NC(Cc2ncccc2Br)c2ccccc21 ; O=[N+]([O-])c1ccc(CBr)cc1Cl ; O=C1c2ccccc2C(Cc2ncccc2Br)N1Cc1ccc([N+](=O)[O-])c(Cl)c1 ; "N2H4" ; O=C1c2ccccc2C(Cc2ncccc2Br)N1Cc1ccc2c(c1)nnn2O]

### Step 1. 4-(bromomethyl)-2-chloro-1-nitrobenzene (288-2)

To a solution of 2-chloro-4-methyl-1-nitrobenzene (500 mg, 2.91 mmol) in CCl₄ (5 mL) were added NBS (571 mg, 3.21 mmol) and AIBN (48 mg, 0.29 mmol) under Ar. Then the mixture was stirred at 90° C. for 30 min using microwave. The reaction progress was monitored by LC/MS. Once completed, the solvent was removed under reduced pressure and the residue was purified by column chromatography (MeOH/DCM=1/20) to give the product as a colorless oil (240 mg, 33%).

### Step 2. 3-((3-bromopyridin-2-yl)methyl)-2-(3-chloro-4-nitrobenzyl)isoindolin-1-one (288-3)

To a solution of 3-[(3-bromo-2-pyridyl)methyl]isoindolin-1-one (290 mg, 0.96 mmol) in DMF (2 mL) was added NaH (46 mg, 1.15 mmol) at 0° C. and the mixture was stirred at 0° C. for 30 min. Then 4-(bromomethyl)-2-chloro-1-nitrobenzene (240 mg, 0.96 mmol) was added, and the mixture was stirred for additional 2h at r.t. The reaction progress was monitored by LC/MS. Once completed, the mixture was diluted with DCM (50 mL) and washed with H₂O (40 mL, 2 times). The organic layer was dried over MgSO₄, filtered and concentrated under reduced pressure. The residue was purified by column chromatography (MeOH/DCM=1/20) to give the product as a light yellow oil (220 mg, 49%). MS (ESI) m/z 472 [M+H]⁺.

### Step 3. 3-((3-bromopyridin-2-yl)methyl)-2-((1-hydroxy-11-benzo[d][1,2,3]triazol-5-yl) methyl)isoindolin-1-one (288)

To a solution of 3-((3-bromopyridin-2-yl)methyl)-2-(3-chloro-4-nitrobenzyl)isoindolin-1-one (50 mg, 0.11 mmol) in EtOH (1 mL) was added a N₂H₄—H₂O solution (80%, 0.1 mL) and the mixture was stirred at 90° C. overnight. The reaction progress was monitored by LC/MS. Once completed, the solvent was removed and the residue was purified by prep-HPLC to give the product as a white solid (8 mg, 16.4%). MS (ESI) m/z 450 [M+H]⁺. ¹H NMR (400 MHz, DMSO-d) δ 8.52 (dd, J=4.6, 1.5 Hz, 1H), 7.98 (dd, J=8.1, 1.5 Hz, 1H), 7.82-7.71 (m, 1H), 7.63-7.55 (m, 2H), 7.51 (dd, J=5.7, 3.1 Hz, 2H), 7.27 (dd, J=8.1, 4.6 Hz, 2H), 7.08-7.00 (m, 1H), 5.21-5.16 (m, 1H), 5.15 (d, J=6.5 Hz, 1H), 4.60 (d, J=15.5 Hz, 1H), 3.59 (dd, J=15.1, 6.0 Hz, 1H), 3.23 (dd, J=15.1, 7.1 Hz, 1H).

### 2-((1H-benzo[d][1,2,3]triazol-5-yl)methyl)isoindolin-1-one (289)

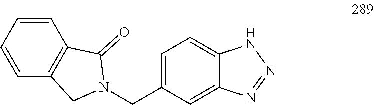 [SMILES: O=C1c2ccccc2CN1Cc1ccc2[nH]nnc2c1]

The title compound 289 was prepared according to general procedure C as a white solid (5 mg, 17.9%). MS (ESI) m/z 265 [M+H]⁺. ¹H NMR (400 MHz, DMSO-d₆) δ 15.31 (s, 1H), 7.88 (d, J=8.5 Hz, 1H), 7.79-7.72 (m, 2H), 7.64-7.47 (m, 3H), 7.34 (d, J=8.5 Hz, 1H), 4.90 (s, 2H), 4.40 (s, 2H).

### 2-((7-fluoro-1H-benzo[d][1,2,3]triazol-5-yl)methyl)-3-methylisoindolin-1-one (290)

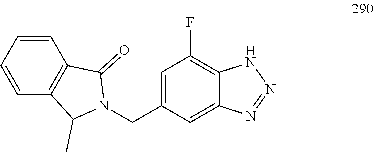 [SMILES: CC1c2ccccc2C(=O)N1Cc1cc(F)c2[nH]nnc2c1]

The title compound 290 was prepared according to general procedure C as a white solid (21 mg, 59%). MS (ESI) m/z 297 [M+H]⁺. ¹H NMR (400 MHz, CD₃OD) δ 7.82 (d, J=7.6 Hz, 1H), 7.61 (t, J=7.2 Hz, 1H), 7.55-7.49 (m, 3H), 6.84-6.82 (m, 1H), 5.27 (d, J=15.2 Hz, 1H), 4.59-4.51 (m, 2H), 1.47 (d, J=7.2 Hz, 3H).

The intermediate 290-6 was prepared as follows:

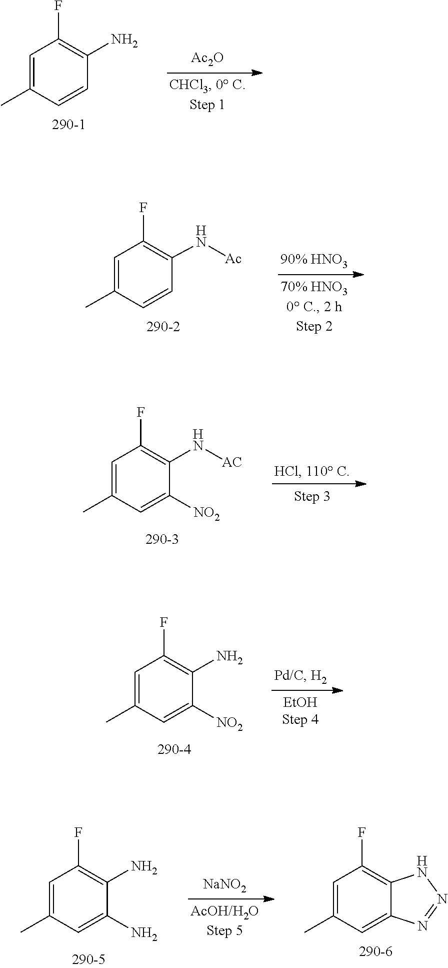

### Step 1. N-(2-fluoro-4-methylphenyl)acetamide (290-2)

To a solution of 2-fluoro-4-methylaniline (10 g, 80 mmol) in CHCl₃ (80 mL) was added Ac₂O (8.1 g, 79.4 mmol) at 0° C. The mixture was stirred for 30 min at 0° C. Water (100 mL) was added, and the solution was neutralized with a sat. Na₂CO₃. The mixture was extracted with EtOAc (100 mL*3). The combined organic layers were washed with water (100 mL), dried over anhydrous Na₂SO₄, filtered and concentrated under vacuum. The residue was washed with PE (100 mL), and the solid was collected by filtration. The solid was dried under vacuum to give N-(2-fluoro-4-methylphenyl) acetamide (13 g, 93%) as a white solid. MS (ESI) m/z 168 [M+H]⁺.

### Step 2. N-(2-fluoro-4-methyl-6-nitrophenyl)acetamide (290-3)

To a solution of N-(2-fluoro-4-methylphenyl)acetamide (6 g, 35.9 mmol) in 70% HNO₃ (50 mL) were added 90% HNO₃ (40 mL) at 0° C. The mixture was stirred for 2 h at 0° C. Water (300 mL) was added, and the solid was collected by filtration. The solid was dried under vacuum to give N-(2-fluoro-4-methyl-6-nitrophenyl)acetamide (7 g, 92%) as a white solid. MS (ESI) m/z 213 [M+H]⁺.

### Step 3. 2-fluoro-4-methyl-6-nitroaniline (290-4)

A solution of N-(2-fluoro-4-methyl-6-nitrophenyl)acetamide (6 g, 28.3 mmol) in 20% HCl (150 mL) was stirred for 4 h at 110° C. The solution was neutralized with a sat. Na₂CO₃. The mixture was extracted with EtOAc (100 mL*3), and the organic layer was separated, dried over anhydrous Na₂SO₄ and concentrated under vacuum. The residue was purified by silica gel flash column chromatography (PE/EtOAc=1/5) to afford 2-fluoro-4-methyl-6-nitroaniline (500 mg, 11%) as a yellow solid. MS (ESI) m/z 171 [M+H]⁺. ¹H NMR (400 MHz, DMSO-d₆) δ 7.68 (s, 1H), 7.37 (d, J=16.0 Hz, 1H), 7.14 (s, 2H), 2.24 (s, 3H).

### Step 4. 3-fluoro-5-methylbenzene-1,2-diamine (290-5)

To a solution of 2-fluoro-4-methyl-6-nitroaniline (490 mg, 2.9 mmol) in EtOH (25 mL) was added Pd/C (49 mg) at r.t. The mixture was stirred for 4 h under an H₂ balloon. The reaction was filtered and concentrated to afford 3-fluoro-5-methylbenzene-1,2-diamine (350 mg, 87%) as a brown solid. MS (ESI) m/z 141 [M+H]⁺.

### Step 5. 7-fluoro-5-methyl-1H-benzo[d][1,2,3]triazole (290-6)

A solution of NaNO₂ (201 mg, 2.9 mmol) in water (2 mL) was added to a solution of 3-fluoro-5-methylbenzene-1,2-diamine (340 mg, 2.4 mmol) in AcOH (15 mL) and H₂O (7 mL) at 0° C. The mixture was stirred at r.t. for 2 h. Water (50 mL) was added, and the solution was neutralized with a sat. Na₂CO₃. The mixture was extracted with EtOAc (50 mL*3). The combined organic layers were washed with brine (100 mL), dried over anhydrous Na₂SO₄, filtered and concentrated under vacuum to give 7-fluoro-5-methyl-1H-benzo[d][1,2,3]triazole (340 mg, 93%) as a yellow solid. MS (ESI) m/z 152 [M+H]⁺. ¹H NMR (400 MHz, CDCl₃) δ 7.41 (s, 1H), 7.31 (s, 1H), 6.99 (d, J=14.0 Hz, 1H), 2.57 (s, 3H).

### 1-(4-((1-methyl-3-oxoisoindolin-2-yl)methyl)phenyl)-3-(pyridin-4-ylmethyl)urea (291)

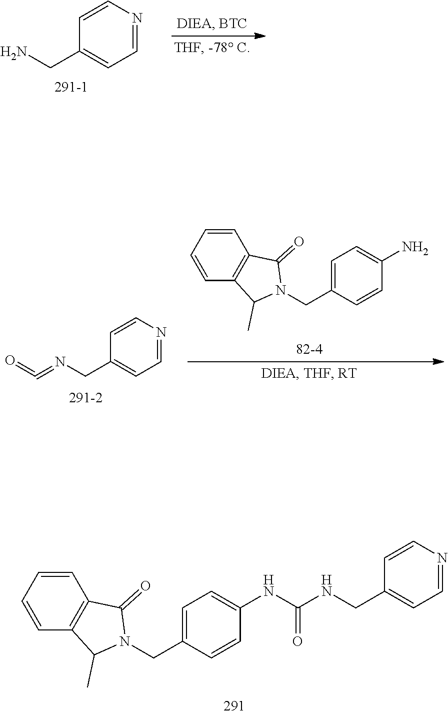 [SMILES: NCc1ccncc1 ; O=C=NCc1ccncc1 ; CC1c2ccccc2C(=O)N1Cc1ccc(N)cc1 ; CC1c2ccccc2C(=O)N1Cc1ccc(NC(=O)NCc2ccncc2)cc1]

### Step 1. 4-(isocyanatomethyl)pyridine (291-2)

To a solution of pyridin-4-ylmethanamine (200 mg, 1.9 mmol) in THF (8 mL) were added DIEA (716 mg, 5.6 mmol) and BTC (220 mg, 0.7 mmol) at −78° C. under N₂. The mixture was stirred for 30 min at −78° C. After the reaction was completed, the mixture was used in the next step without work up.

### Step 2. 1-(4-((1-methyl-3-oxoisoindolin-2-yl)methyl)phenyl)-3-(pyridin-4-ylmethyl)urea (291)

To a solution of 2-(4-aminobenzyl)-3-methylisoindolin-1-one (40 mg, 1 mmol) and DIEA (42 mg, 0.3 mmol) in THF (5 mL) was added a solution of 4-(isocyanatomethyl)pyridine in THF (2 mL) that was made in step 1 at −40° C. under N₂. The mixture was stirred overnight at r.t. Water (10 mL) was added, and the reaction mixture was extracted with EtOAc (20 mL*2). The combined organic layers were washed with brine (20 mL), dried over anhydrous Na₂SO₄, filtered and concentrated under vacuum. The residue was purified by prep-HPLC to give 1-(4-((1-methyl-3-oxoisoindolin-2-yl)methyl)phenyl)-3-(pyridin-4-ylmethyl)urea (7 mg, 12%) as a white solid. MS (ESI) m/z 387 [M+H]⁺. ¹H NMR (400 MHz, DMSO-d₆) b 8.71 (s, 1H), 8.48 (dd, J=4.4, 1.6 Hz, 2H), 7.70 (d, J=7.6 Hz, 1H), 7.61-7.57 (m, 2H), 7.52-7.48 (m, 1H), 7.36 (d, J=8.4 Hz, 2H), 7.26 (d, J=5.6 Hz, 2H), 7.16 (d, J=8.4 Hz, 2H), 6.70 (t, J=6.0 Hz, 1H), 4.95 (d, J=14.8 Hz, 1H), 4.39 (q, J=6.4 Hz, 1H), 4.31-4.28 (m, 3H), 1.37 (d, J=6.8 Hz, 3H).

### 1-((1H-pyrazol-4-yl)methyl)-3-(4-((1-methyl-3-oxoisoindolin-2-yl)methyl)phenyl)urea (292)

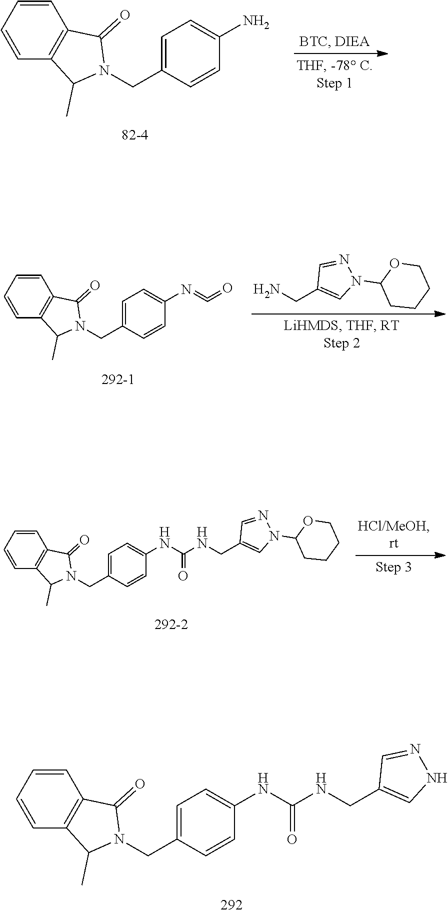 [SMILES: CC1c2ccccc2C(=O)N1Cc1ccc(N)cc1 ; CC1c2ccccc2C(=O)N1Cc1ccc(N=C=O)cc1 ; NCc1cnn(C2CCCCO2)c1 ; CC1c2ccccc2C(=O)N1Cc1ccc(NC(=O)NCc2cnn(C3CCCCO3)c2)cc1 ; CC1c2ccccc2C(=O)N1Cc1ccc(NC(=O)NCc2cn[nH]c2)cc1]

### Step 1. 2-(4-isocyanatobenzyl)-3-methylisoindolin-1-one (292-1)

To a solution of 2-(4-aminobenzyl)-3-methylisoindolin-1-one (40 mg, 0.2 mmol) in THF (5 mL) were added DIEA (62 mg, 0.5 mmol) and BTC (18 mg, 0.1 mmol) at −78° C. under N₂. The mixture was stirred for 30 min at −78° C. After the reaction was completed, the mixture was used for the next step without work up.

### Step 2. 1-(4-((1-methyl-3-oxoisoindolin-2-yl)methyl)phenyl)-3-((1-(tetrahydro-2H-pyran-2-yl)-1H-pyrazol-4-yl)methyl)urea (292-2)

To a solution of (1-(tetrahydro-2H-pyran-2-yl)-1H-pyrazol-4-yl)methanamine (45 mg, 0.3 mmol) in THF (5 mL) was added LiHMDS (0.2 mL, 1.0 M in hexane, 0.2 mmol) at −78° C. The reaction was stirred at −78° C. for 10 min. A solution of 2-(4-isocyanatobenzyl)-3-methylisoindolin-1-one that was made in Step 1 was added to the reaction at −78° C. under N₂. The mixture was stirred at r.t. for 2 h. Water (10 mL) was added, and extracted with EtOAc (20 mL*2). The combined organic layers were washed with brine (20 mL), dried over anhydrous Na₂SO₄, filtered and concentrated under vacuum. The residue was purified by prep-HPLC to give 1-(4-((1-methyl-3-oxoisoindolin-2-yl)methyl)phenyl)-3-((1-(tetrahydro-2H-pyran-2-yl)-1H-pyrazol-4-yl)methyl)urea (15 mg, 20%) as a white solid. MS (ESI) m/z 460 [M+H]⁺.

### Step 3. 1-((1H-pyrazol-4-yl)methyl)-3-(4-((1-methyl-3-oxoisoindolin-2-yl)methyl) phenyl) urea (292)

To a solution of 1-(4-((1-methyl-3-oxoisoindolin-2-yl)methyl)phenyl)-3-((1-(tetrahydro-2H-pyran-2-yl)-1H-pyrazol-4-yl)methyl)urea (15 mg, 0.03 mmol) in MeOH (2 mL) was added HCl (0.2 mL) at r.t. The reaction was stirred for 2 h at r.t. The mixture was concentrated under vacuum and the residue was purified by prep-HPLC to give 1-((1H-pyrazol-4-yl)methyl)-3-(4-((1-methyl-3-oxoisoindolin-2-yl)methyl)phenyl)urea (5 mg, 42%) as a white solid. MS (ESI) m/z 376 [M+H]⁺. ¹H NMR (400 MHz, DMSO-d₆) δ 12.65 (brs, 1H), 8.44 (s, 1H), 7.70 (d, J=7.6 Hz, 1H), 7.62-7.57 (m, 3H), 7.51-7.41 (m, 2H), 7.34 (d, J=8.4 Hz, 2H), 7.15 (d, J=8.4 Hz, 2H), 6.29 (t, J=5.2 Hz, 1H), 4.95 (d, J=15.2 Hz, 1H), 4.39 (q, J=6.4 Hz, 1H), 4.28 (d, J=15.2 Hz, 1H), 4.12 (d, J=5.6 Hz, 2H), 1.38 (d, J=6.8 Hz, 3H).

### 1-(4-((1-oxoisoindolin-2-yl)methyl)phenyl)urea (293)

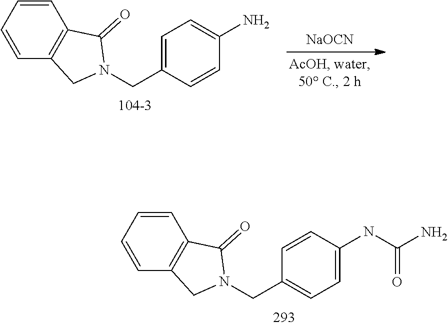

To a solution of 2-(4-aminobenzyl)isoindolin-1-one (100 mg, 0.42 mmol) in acetic acid (0.5 mL) and water (1 mL) was added sodium cyanate (55 mg, 0.83 mmol) under N₂. The mixture was stirred at 50′C for 2 h, and the mixture was filtered and then purified by C18 column (ACN=40%) to give 1-(4-((1-oxoisoindolin-2-yl)methyl)phenyl)urea (16 mg, 13%) as an off-white solid. MS (ESI) m/z 282 [M+H]⁺. ¹H NMR (400 MHz, DMSO-d₆) δ 8.57 (s, 1H), 7.71 (d, J=7.5, 1H), 7.63-7.53 (m, 2H), 7.51-7.47 (m, 1H), 7.42-7.32 (m, 2H), 7.18-7.10 (m, 2H), 5.83 (s, 2H), 4.63 (s, 2H), 4.33 (s, 2H).

### methyl (4-((1-oxoisoindolin-2-yl)methyl)phenyl)carbamate (294)

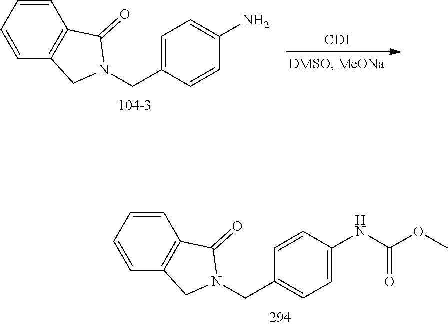 [SMILES: Nc1ccc(CN2Cc3ccccc3C2=O)cc1 ; "CDI" ; COC(=O)Nc1ccc(CN2Cc3ccccc3C2=O)cc1]

To a solution of 2-(4-aminobenzyl)isoindolin-1-one (100 mg, 0.42 mmol) in DMSO (1 mL) was added CDI (136 mg, 0.84 mmol) at 0° C. under N₂. The mixture was stirred for 30 min. Sodium methanolate (25 mg, 0.46 mmol) was added and the reaction was allowed to warm to r.t. This suspension was allowed to stir for an additional 2h. Water was added at 0° C. and then the reaction mixture was extracted with EtOAc. The organic layer was separated, dried over anhydrous Na₂SO₄ and concentrated under vacuum. The residue was purified by prep HPLC to give methyl (4-((1-oxoisoindolin-2-yl)methyl) phenyl)carbamate (25 mg, 19%) as a white solid. MS (ESI) m/z 297 [M+H]⁺. ¹H NMR (400 MHz, DMSO-d₆) δ 9.64 (s, 1H), 7.75-7.68 (m, 1H), 7.63-7.53 (m, 2H), 7.53-7.45 (m, 1H), 7.45-7.38 (m, 2H), 7.23-7.16 (m, 2H), 4.65 (s, 2H), 4.34 (s, 2H), 3.64 (s, 3H).

### 2-(4-hydroxybenzyl)-4-methylisoindoline-1,3-dione (295)

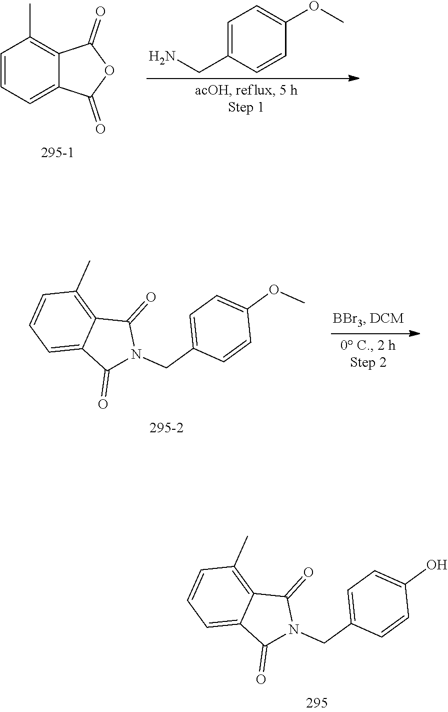

### Step 1. 2-(4-methoxybenzyl)-4-methylisoindoline-1,3-dione (295-2)

To a stirred solution of 4-methylisobenzofuran-1,3-dione (200 mg, 1.23 mmol) in AcOH (5 mL) was added (4-methoxyphenyl)methanamine (169 mg, 1.23 mmol), and the reaction mixture was refluxed for 5 h. The reaction mixture was quenched with water, and extracted with EA (3×50 mL). The combined organic phase was washed with brine, dried over anhydrous Na₂SO₄ and concentrated to afford the title compound (345 mg, crude) as a light yellow solid. MS (ESI) m/z 282 [M+H]⁺.

### Step 2. 2-(4-hydroxybenzyl)-4-methylisoindoline-1,3-dione (295)

To a solution of 2-(4-methoxybenzyl)-4-methylisoindoline-1,3-dione (345 mg, 1.23 mmol) in DCM (5 mL) was added BBr₃ (923 mg, 3.69 mmol) at 0° C. and reacted for 2h, and then the reaction mixture was quenched with water, and extracted with DCM (3×50 mL). The combined organic phase was washed with brine, dried over anhydrous Na₂SO₄ and concentrated. The residue was purified by Prep-HPLC to afford 2-(4-hydroxybenzyl)-4-methylisoindoline-1,3-dione (80 mg, 24.4%) as a white solid. MS (ESI) m/z 268 [M+H]⁺. ¹H NMR (400 MHz, DMSO-d₆) δ 9.38 (d, J=4.0 Hz, 1H), 7.74-7.65 (m, 2H), 7.65-7.58 (m, 1H), 7.15-7.09 (m, 2H), 6.73-6.66 (m, 2H), 4.62 (s, 2H), 2.62 (s, 3H).

### 4-bromo-2-(4-hydroxybenzyl)isoindoline-1,3-dione (296)

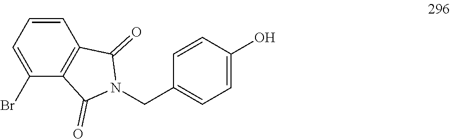 [SMILES: O=C1c2cccc(Br)c2C(=O)N1Cc1ccc(O)cc1]

The title compound 296 was prepared according to general procedure D as a white solid (15 mg, 21%). MS (ESI) m/z 332 [M+H]⁺. ¹H NMR (400 MHz, DMSO-d₆) δ 9.39 (s, 1H), 8.00 (d, J=8.0 Hz, 1H), 7.88 (d, J=7.3 Hz, 1H), 7.72 (t, J=7.7 Hz, 1H), 7.13 (d, J=8.4 Hz, 2H), 6.70 (d, J=8.4 Hz, 2H), 4.63 (s, 2H).

### 5-methyl-2-((2-oxo-2,3-dihydrobenzo[d]oxazol-6-yl)methyl)isoindoline-1,3-dione (297)

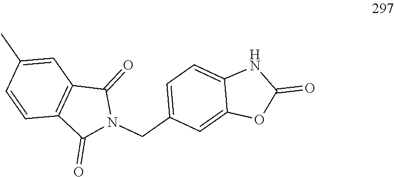 [SMILES: Cc1ccc2c(c1)C(=O)N(Cc1ccc3[nH]c(=O)oc3c1)C2=O]

The title compound 297 was prepared according to general procedure B as a white solid (9 mg, 40%). MS (ESI) m/z 309 [M+H]⁺. ¹H NMR (400 MHz, DMSO-d₆) δ 11.60 (s, 1H), 7.77 (d, J=7.6 Hz, 1H), 7.72 (s, 1H), 7.65 (d, J=7.4 Hz, 1H), 7.24 (s, 1H), 7.09 (d, J=8.0, 1H), 7.03 (d, J=8.0 Hz, 1H), 4.75 (s, 2H), 2.48 (s, 3H).

### 2-(4-hydroxybenzyl)-5-(pyrrolidin-1-yl)isoindolin-1-one (298)

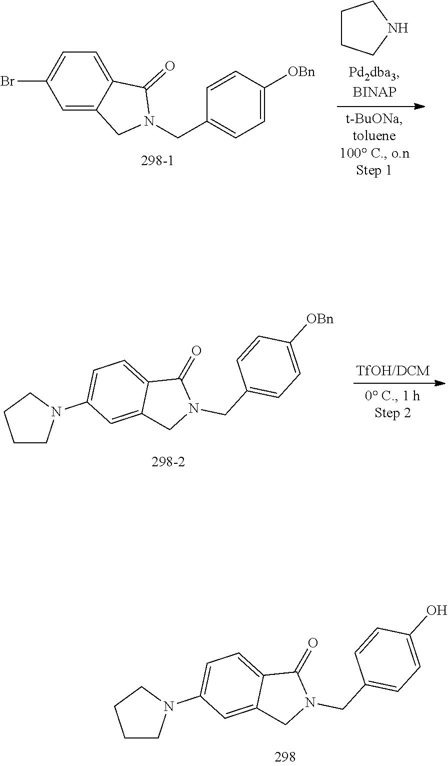 [SMILES: O=C1c2ccc(Br)cc2CN1Cc1ccc(OCc2ccccc2)cc1 ; C1CCNC1 ; O=C1c2ccc(N3CCCC3)cc2CN1Cc1ccc(OCc2ccccc2)cc1 ; O=C1c2ccc(N3CCCC3)cc2CN1Cc1ccc(O)cc1]

298-1 was prepared according to the procedure described for 1-3.

Step 1 was performed according to the procedure described for step 1 for the preparation of 47-2.

### Step 2. 2-(4-hydroxybenzyl)-5-(pyrrolidin-1-yl)isoindolin-1-one (298)

To a stirred solution of 2-(4-(benzyloxy) benzyl)-5-(pyrrolidin-1-yl)isoindolin-1-one (60 mg, 0.15 mmol) in DCM (5 mL) was added TfOH (0.5 mL) at 0° C. The resulting mixture was stirred at 0° C. for 1 h. The mixture was quenched by a sat. NaHCO₃ (aq.), and extracted with DCM (3×30 mL). The combined organic phase was dried (Na₂SO₄) and concentrated. The residue was purified by Prep-HPLC to afford a white solid (10 mg, 21.5%). MS (ESI) m/z 309 [M+H]⁺. ¹H NMR (400 MHz, DMSO-d₆) δ 9.35 (s, 1H), 7.45 (d, J=8.3 Hz, 1H), 7.05 (d, J=8.2 Hz, 2H), 6.71 (d, J=8.2 Hz, 2H), 6.62-6.57 (m, 2H), 4.51 (s, 2H), 4.16 (s, 2H), 3.29-3.24 (m, 4H), 2.00-1.90 (m, 4H).

### 6-((5-bromo-1-oxoisoindolin-2-yl)methyl)benzo[d]oxazol-2(3H)-one (299)

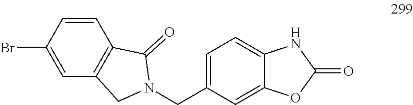 [SMILES: O=C1c2ccc(Br)cc2CN1Cc1ccc2[nH]c(=O)oc2c1]

The title compound 299 was prepared according to general procedure B as an off-white solid (42 mg, 13%). MS (ESI) m/z 361 [M+H]+¹H NMR (400 MHz, DMSO-d₆) δ 11.55 (s, 1H), 6 7.81 (s, 1H), 7.71-7.63 (m, 2H), 7.22 (s, 1H), 7.11-7.02 (m, 2H), 4.71 (s, 2H), 4.36 (s, 2H).

### 6-((1-oxo-5-(pyrrolidin-1-yl)isoindolin-2-yl)methyl)benzo[d]oxazol-2(3H)-one (300)

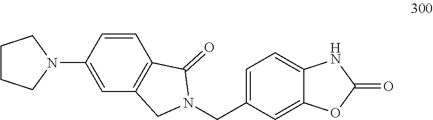 [SMILES: O=C1c2ccc(N3CCCC3)cc2CN1Cc1ccc2[nH]c(=O)oc2c1]

The title compound 300 was prepared according to the procedure described for compound 47 as a white solid (3 mg, 6%) as a white solid. MS (ESI) m/z 350 [M+H]⁺. ¹H NMR (400 MHz, DMSO-d₆) δ 11.59 (s, 1H), 7.47 (d, J=8.3 Hz, 1H), 7.17 (s, 1H), 7.06-7.02 (m, 2H), 6.63-6.58 (m, 2H), 4.64 (s, 2H), 4.21 (s, 2H), 3.29-3.25 (m, 4H), 2.00-1.92 (m, 4H).

### 2-((1H-benzo[d][1,2,3]triazol-5-yl)methyl)-1-methyl-1,2-dihydro-3H-indazol-3-one (301)

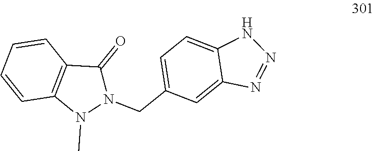 [SMILES: Cn1c2ccccc2c(=O)n1Cc1ccc2[nH]nnc2c1]

The title compound 301 was prepared according to the procedure described for compound 46 as a white solid (13 mg, 25%). MS (ESI) m/z 280 [M+H]⁺. ¹H NMR (400 MHz, DMSO-d₆) δ 15.54 (s, 1H), 7.84 (d, J=8.6 Hz, 1H), 7.78 (s, 1H), 7.76-7.71 (m, 1H), 7.61-7.55 (m, 1H), 7.46 (d, J=8.3 Hz, 1H), 7.29 (dd, J=8.6, 1.5 Hz, 1H), 7.23-7.15 (m, 1H), 5.25 (s, 2H), 3.28 (s, 3H).

### 6-((1-methyl-5-(methyl(2-azaspiro[3.3]heptan-6-yl)amino)-3-oxoisoindolin-2-yl)methyl) benzo[d]oxazol-2(3H)-one (302) and 6-((3-methyl-5-(methyl(2-azaspiro[3.3]heptan-6-yl)amino)-1-oxoisoindolin-2-yl)methyl)benzo[d]oxazol-2(3H)-one (303)

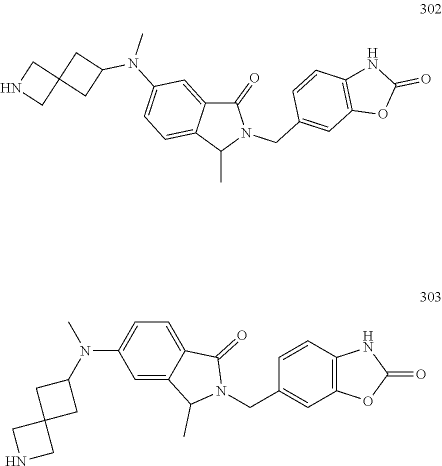 [SMILES: CC1c2ccc(N(C)C3CC4(CNC4)C3)cc2C(=O)N1Cc1ccc2[nH]c(=O)oc2c1 ; CC1c2cc(N(C)C3CC4(CNC4)C3)ccc2C(=O)N1Cc1ccc2[nH]c(=O)oc2c1]

The title compounds 302 and 303 were prepared according to the procedure described for compounds 50 and 51. After pre-HPLC separation, two compounds were obtained: compound 302 and compound 303. One of the two compounds was a yellow solid (6 mg, 7.2%), with RT=4.16 min (HPLC), MS (ESI) m/z 419 [M+H]⁺. ¹H NMR (400 MHz, DMSO-d₆) δ 11.62 (s, 1H), 7.36 (d, J=8.4 Hz, 1H), 7.20 (s, 1H), 7.08-6.97 (m, 4H), 4.99 (d, J=15.2 Hz, 1H), 4.37 (d, J=15.2 Hz, 1H), 4.30 (q, J=6.4 Hz, 1H), 4.09 (t, J=6.0 Hz, 2H), 4.04-3.96 (m, 1H), 3.90 (t, J=6.0 Hz, 2H), 2.79 (s, 3H), 2.60-2.54 (m, 2H), 2.29-2.23 (m, 2H), 1.36 (d, J=6.7 Hz, 3H). The other one of the two compounds was a white solid (3 mg, 3.6%), with RT=4.82 min (HPLC), MS (ESI) m/z 419 [M+H]⁺. ¹H NMR (400 MHz, DMSO-d₆) δ 11.61 (s, 1H), 7.48 (d, J=9.2 Hz, 1H), 7.19 (s, 1H), 7.07-7.02 (m, 2H), 6.85-6.82 (m, 2H), 4.94 (d, J=15.2 Hz, 1H), 4.35 (d, J=15.2 Hz, 1H), 4.27 (q, J=6.4 Hz, 1H), 4.14-4.10 (m, 1H), 4.07 (t, J=5.6 Hz, 2H), 3.90 (t, J=6.0 Hz, 2H), 2.84 (s, 3H), 2.61-2.54 (m, 2H), 2.32-2.26 (m, 2H), 1.36 (d, J=6.7 Hz, 3H). rel-(S)-6-((5-cyclopentyl-3-methyl-1-oxoisoindolin-2-yl)methyl)benzo[d]oxazol-2(3H)-one (304)

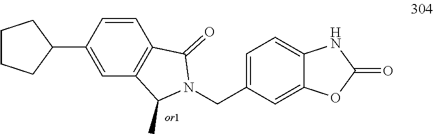 [SMILES: C[C@H]1c2cc(C3CCCC3)ccc2C(=O)N1Cc1ccc2[nH]c(=O)oc2c1]

The title compound 304 was prepared according to the procedure described for compound 87 using the stereoisomer of Int 10 or Int 11 that has RT of 1.969 min as a starting material to afford a white solid (14 mg, 22%). MS (ESI) m/z 363 [M+H]⁺. ¹H NMR (400 MHz, CD₃OD) δ 7.73 (d, J=8.2 Hz, 1H), 7.44-7.41 (m, 2H), 7.23 (d, J=1.6 Hz, 1H), 7.17 (dd, J=8.0, 1.6 Hz, 1H), 7.06 (d, J=8.0 Hz, 1H), 5.17 (d, J=15.4, 1H), 4.52-4.46 (m, 2H), 3.19-3.10 (m, 1H), 2.17-2.07 (m, 2H), 1.96-1.60 (m, 6H), 1.47 (d, J=6.7 Hz, 3H).

### 6-(dimethylamino)-2-(4-hydroxybenzyl) isoindolin-1-one (305)

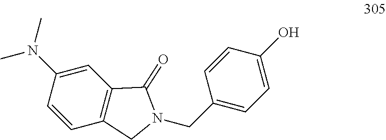 [SMILES: CN(C)c1ccc2c(c1)C(=O)N(Cc1ccc(O)cc1)C2]

The title compound 305 was prepared according to the procedure described for compound 298 as a yellow solid (3 mg, 19.7%). MS (ESI) m/z 283 [M+H]⁺. ¹H NMR (400 MHz, CD₃OD) δ 7.31 (d, J=8.4 Hz, 1H), 7.15-7.10 (m, 3H), 7.01 (dd, J=8.4, 2.4 Hz, 1H), 6.76 (d, J=8.2 Hz, 2H), 4.68 (s, 2H), 4.22 (s, 2H), 2.99 (s, 6H).

### 6-((1-(difluoro(phenyl)methyl)-3-oxoisoindolin-2-yl)methyl)benzo[d]oxazol-2(3H)-one (306)

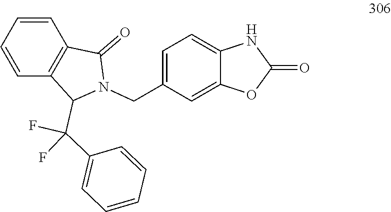 [SMILES: O=C1c2ccccc2C(C(F)(F)c2ccccc2)N1Cc1ccc2[nH]c(=O)oc2c1]

The title compound 306 was prepared according to general procedure E as a white solid (6 mg, 37.6%). MS (ESI) m/z 407 [M+H]⁺. 1H NMR (400 MHz, CD₃OD) δ 7.56 (d, J=7.4 Hz, 1H), 7.50 (td, J=7.6, 1.3 Hz, 1H), 7.42 (td, J=7.5, 1.1 Hz, 1H), 7.38-7.32 (m, 1H), 7.29 (t, J=7.4 Hz, 1H), 7.17 (t, J=7.9 Hz, 2H), 7.08-6.90 (m, 5H), 5.29 (d, J=15.1 Hz, 1H), 5.02 (t, J=7.1 Hz, 1H), 4.40 (d, J=15.1 Hz, 1H).

### 2-((1-((4-chloro-1-methyl-1H-pyrazol-5-yl)methyl)-3-oxoisoindolin-2-yl)methyl)-5-oxa-7-azaspiro[3.4]octan-6-one (307)

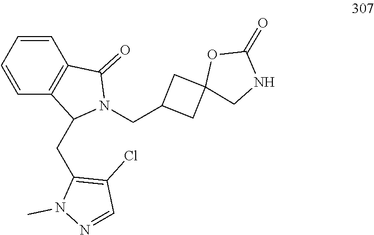 [SMILES: Cn1ncc(Cl)c1CC1c2ccccc2C(=O)N1CC1CC2(CNC(=O)O2)C1]

The title compound 307 was prepared according to the procedure described for compound 270 as a white solid (80 mg, 76.8%). MS (ESI) m/z 401 [M+H]⁺. ¹H NMR (400 MHz, CDCl₃) δ 7.86 (d, J=7.2 Hz, 1H), 7.53 (s, 1H), 7.51-7.46 (m, 1H), 7.45-7.40 (m, 1H), 6.54 (d, J=7.5 Hz, 1H), 4.99 (s, 1H), 4.85 (dd, J=9.8, 5.6 Hz, 1H), 4.23 (dd, J=14.3, 5.3 Hz, 1H), 3.62 (s, 2H), 3.50 (dd, J=14.4, 5.6 Hz, 1H), 3.46 (s, 3H), 3.20 (dd, J=14.4, 6.7 Hz, 1H), 2.59 (dd, J=14.3, 9.8 Hz, 1H), 2.50-2.30 (m, 5H).

The intermediate 307-2 was prepared as follows:

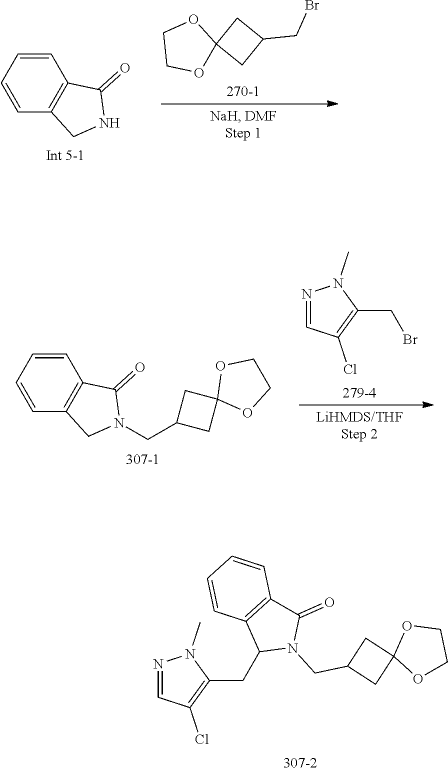

Step 1 was performed according to the procedure outlined for the preparation of Int 5.

Step 2 was performed according to the procedure outlined for step 2 for the preparation of Int 12.

### 4-methyl-3-((3-oxo-2-((6-oxo-5-oxa-7-azaspiro[3.4]octan-2-yl)methyl)isoindolin-1-yl)methyl)picolinonitrile (308)

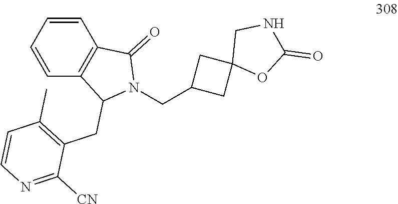 [SMILES: Cc1ccnc(C#N)c1CC1c2ccccc2C(=O)N1CC1CC2(CNC(=O)O2)C1]

The title compound 308 was prepared according to the procedure described for compound 307 as a white solid (100 mg, 76.3%). MS (ESI) m/z 403 [M+H]⁺. ¹H NMR (400 MHz, CDCl₃) δ 8.58 (d, J=4.9 Hz, 1H), 7.89 (d, J=7.6 Hz, 1H), 7.47 (t, J=7.5 Hz, 1H), 7.37 (d, J=5.0 Hz, 1H), 7.33 (dt, J=7.5, 1.3 Hz, 1H), 6.37 (d, J=7.6 Hz, 1H), 5.15 (s, 1H), 4.92 (dd, J=10.1, 5.7 Hz, 11H), 4.24 (dd, J=14.2, 7.9 Hz, 1H), 3.71 (dd, J=13.7, 5.7 Hz, 11H), 3.66-3.60 (m, 2H), 3.36 (dd, J=14.2, 5.8 Hz, 1H), 2.76 (dd, J=13.6, 10.2 Hz, 1H), 2.60-2.31 (m, 5H), 1.98 (s, 3H).

The intermediate 308-3 was prepared as follows:

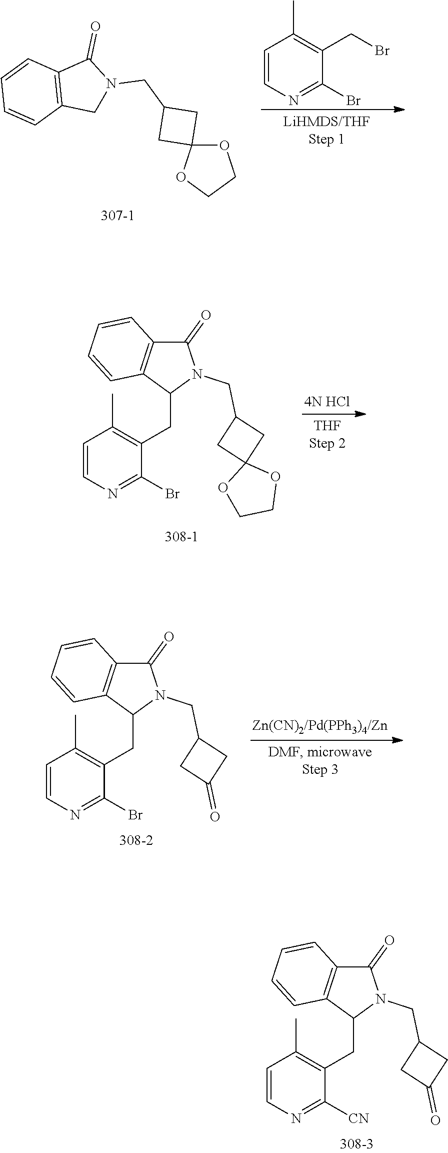

Step 1 was performed according to the procedure outlined for step 2 for the preparation of Int 12.

Step 2 was performed according to the procedure outlined for step 2 for the preparation of compound 270.

Step 3 was performed according to the procedure outlined for step 3 for the preparation of compound 278.

### 2-((1-((4-chloro-1-methyl-1H-pyrazol-5-yl)methyl)-4-fluoro-3-oxoisoindolin-2-yl)methyl)-5-oxa-7-azaspiro[3.4]octan-6-one (309)

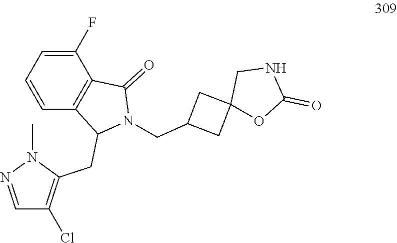 [SMILES: Cn1ncc(Cl)c1CC1c2cccc(F)c2C(=O)N1CC1CC2(CNC(=O)O2)C1]

The title compound 309 was prepared according to the procedure described for compound 307 using 7-fluoroisoindolin-1-one as a starting material to afford a white solid (110 mg, 86.5%). MS (ESI) m/z 419 [M+H]⁺. ¹H NMR (400 MHz, CDCl₃) δ 7.53 (s, 1H), 7.40 (td, J=7.9, 4.7 Hz, 1H), 7.11 (t, J=8.7 Hz, 1H), 6.32 (d, J=7.6 Hz, 1H), 5.25 (s, 1H), 4.85 (dd, J=9.7, 5.5 Hz, 1H), 4.18 (dd, J=14.3, 4.5 Hz, 1H), 3.62 (s, 2H), 3.49 (s, 3H), 3.49 (dd, J=14.3, 5.4 Hz, 1H), 3.23-3.11 (m, 1H), 2.62 (dd, J=14.4, 9.8 Hz, 1H), 2.51-2.28 (m, 5H).

### 2-((1-((4-chloro-1-methyl-1H-pyrazol-5-yl)methyl)-5-fluoro-3-oxoisoindolin-2-yl)methyl)-5-oxa-7-azaspiro[3.4]octan-6-one (310)

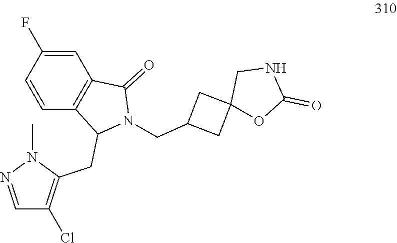 [SMILES: Cn1ncc(Cl)c1CC1c2ccc(F)cc2C(=O)N1CC1CC2(CNC(=O)O2)C1]

The title compound 310 was prepared according to the procedure described for compound 307 using 6-fluoroisoindolin-1-one as a starting material to afford a white solid (90 mg, 82.6%). MS (ESI) m/z 419 [M+H]⁺. ¹H NMR (400 MHz, CDCl₃) δ 7.58-7.47 (m, 2H), 7.13 (td, J=8.6, 2.5 Hz, 1H), 6.47 (dd, J=8.4, 4.4 Hz, 1H), 5.24 (s, 1H), 4.82 (dd, J=9.9, 5.4 Hz, 1H), 4.21 (dd, J=14.5, 5.0 Hz, 1H), 3.62 (s, 2H), 3.57-3.48 (m, 4H), 3.22 (dd, J=14.6, 6.3 Hz, 1H), 2.57 (dd, J=14.3, 9.9 Hz, 1H), 2.52-2.29 (m, 5H).

### 2-((3-((4-chloro-1-methyl-1H-pyrazol-5-yl)methyl)-5-cyclopropyl-1-oxoisoindolin-2-yl)methyl)-5-oxa-7-azaspiro[3.4]octan-6-one (3H)

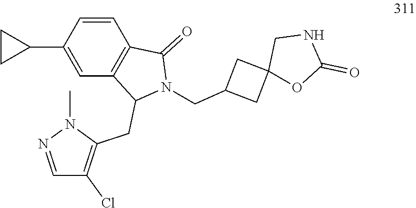 [SMILES: Cn1ncc(Cl)c1CC1c2cc(C3CC3)ccc2C(=O)N1CC1CC2(CNC(=O)O2)C1]

The title compound 311 was prepared according to the procedure described for compound 307 using 5-cyclopropylisoindolin-1-one as a starting material to afford a white solid (51 mg, 19.2%). MS (ESI) m/z 441 [M+H]⁺. ¹H NMR (400 MHz, CDCl₃) δ 7.71 (d, J=7.9 Hz, 1H), 7.53 (s, 1H), 7.19 (dd, J=7.9, 1.6 Hz, 1H), 6.05 (s, 1H), 5.02 (s, 1H), 4.76 (dd, J=9.8, 5.7 Hz, 1H), 4.20 (dd, J=14.3, 5.4 Hz, 1H), 3.61 (s, 2H), 3.48 (dd, J=14.3, 5.7 Hz, 1H), 3.43 (s, 3H), 3.16 (dd, J=14.3, 6.8 Hz, 1H), 2.54 (dd, J=14.3, 9.8 Hz, 1H), 2.46-2.41 (m, 2H), 2.40-2.28 (m, 3H), 1.90-1.82 (m, 1H), 1.08-0.91 (m, 2H), 0.69-0.50 (m, 2H).

The intermediate 311-6 was prepared as follows:

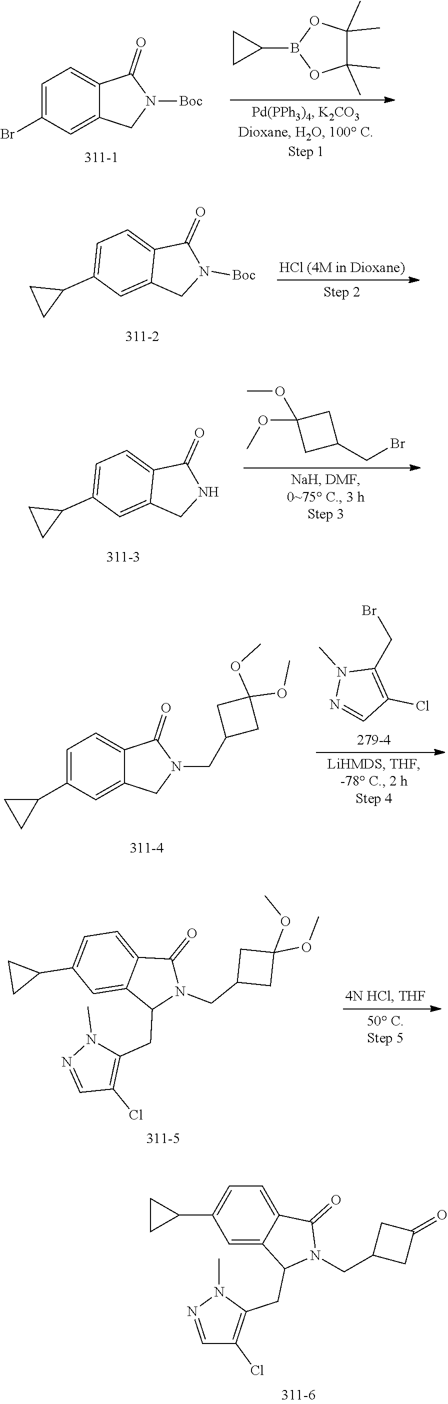

Step 1 was performed according to the procedure outlined for step 1 for the preparation of compound 181

### Step 2. 5-cyclopropylisoindolin-1-one (311-3)

A solution of 311-2 (700 mg, 2.56 mmol) in HCl (4M in dioxane, 8 mL) was stirred at r.t. for 2 h. The precipitated solid was collected by filtration and washed with dioxane. The collected solid was dried to afford the title compound (425 mg, 95.8%) as a white solid. MS (ESI) m/z 174 [M+H]⁺.

Step 3 was performed according to the procedure outlined for the preparation of Int 5.

Step 4 was performed according to the procedure outlined for step 2 for the preparation of Int 12.

Step 5 was performed according to the procedure outlined for step 2 for the preparation of compound 270.

### (S,E)-2-(3-(1H-1,2,3-triazol-4-yl)allyl)-3-((4-chloro-1-methyl-1H-pyrazol-5-yl)methyl) isoindolin-1-one (312) and (R,E)-2-(3-(1H-1,2,3-triazol-4-yl)allyl)-3-((4-chloro-1-methyl-1H-pyrazol-5-yl)methyl)isoindolin-1-one (313)

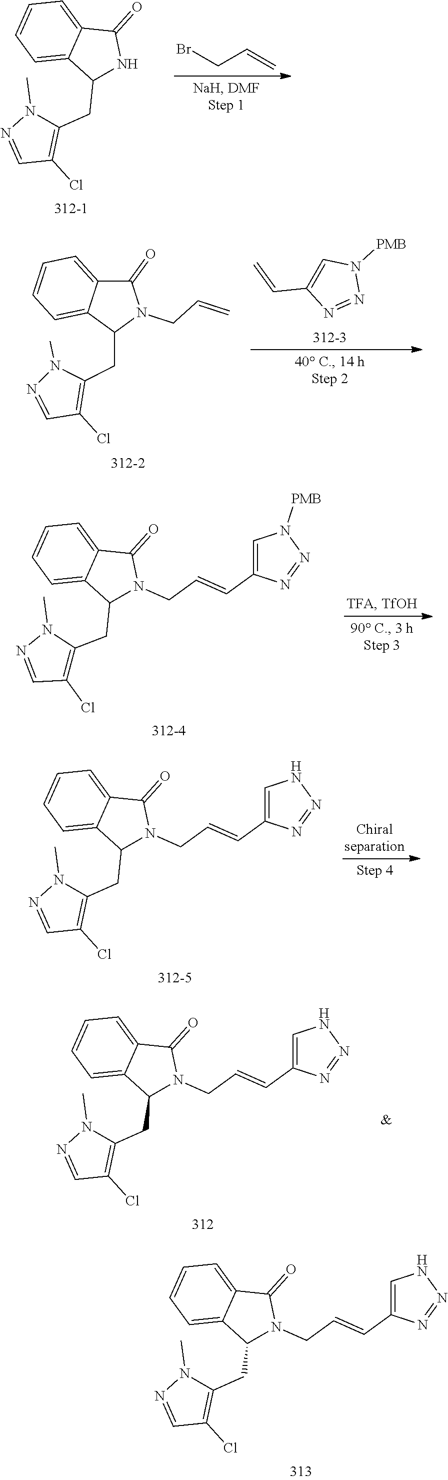

312-1 was prepared according to the procedure outlined for In12.

### Step 1. 2-allyl-3-((4-chloro-1-methyl-1H-pyrazol-5-yl)methyl)isoindolin-1-one (312-2)

To a solution of 312-1 (1.1 g, 4.2 mmol) in DMF (10 mL) was added NaH (111 mg, 4.62 mmol) at 0° C. under N₂. The mixture was stirred for 30 min. Then 3-bromoprop-1-ene (559 mg, 4.62 mmol) was added and the reaction mixture was allowed to warm to r.t. This suspension was stirred for an additional 2 h. The mixture was purified by C18 column (70% ACN) to give 2-allyl-3-((4-chloro-1-methyl-1H-pyrazol-5-yl)methyl)isoindolin-1-one (880 mg, 69%) as a yellow solid. MS (ESI) m/z 302 [M+H]⁺.

### Step 2. (E)-3-((4-chloro-1-methyl-1H-pyrazol-5-yl)methyl)-2-(3-(1-(4-methoxybenzyl)-1H-1,2,3-triazol-4-yl)allyl)isoindolin-1-one (312-4)

To a solution of 312-3 (628 mg, 2.92 mmol) in DCE (10 mL) were added 312-2 (880 mg, 2.92 mmol) and Hoveyda-Grubbs 2^{nd} (183 mg, 0.29 mmol) under N₂. The mixture was stirred at 40° C. for 14 h. The mixture was purified by silica gel column chromatography (DCM/MeOH=15/1) to give (E)-3-((4-chloro-1-methyl-1H-pyrazol-5-yl)methyl)-2-(3-(1-(4-methoxybenzyl)-1H-1,2,3-triazol-4-yl)allyl)isoindolin-1-one (450 mg, 31%) as a black solid. MS (ESI) m/z 489 [M+H]⁺.

### Step 3. (E)-2-(3-(1H-1,2,3-triazol-4-yl)allyl)-3-((4-chloro-1-methyl-1H-pyrazol-5-yl)methyl) isoindolin-1-one (312-5)

To a solution of 312-4 (650 mg, 1.33 mmol) in TFA (6 mL) was added TfOH (1.5 mL). The mixture was stirred at 90° C. for 3 h. The mixture was concentrated under vacuum. The residue was taken in DCM, then the pH was adjusted to 7 with sat. NaHCO₃ (aq.). The organic layer was separated, dried over anhydrous Na₂SO₄ and concentrated in vacuo. The residue was purified by prep-TLC (DCM/MeOH=10/1) to give (E)-2-(3-(1H-1,2,3-triazol-4-yl)allyl)-3-((4-chloro-1-methyl-1H-pyrazol-5-yl)methyl)isoindolin-1-one (200 mg, 40%) as a brown solid. MS (ESI) m/z 369 [M+H]⁺.

### Step 4. (S,E)-2-(3-(1H-1,2,3-triazol-4-yl)allyl)-3-((4-chloro-1-methyl-1H-pyrazol-5-yl)methyl)isoindolin-1-one (312) and (R,E)-2-(3-(1H-1,2,3-triazol-4-yl)allyl)-3-((4-chloro-1-methyl-1H-pyrazol-5-yl)methyl)isoindolin-1-one (313)

Chiral separation of 312-5 by chiral prep-HPLC (column: CHIRALPAK IH; column size: 2 cm×25 cm, 5 μm; mobile phase A: MtBE; mobile phase B: EtOH) was conducted to afford two enantiomers: compound 312 and compound 313. 313 was a yellow solid, with RT=1.329 min (CHIRAL HPLC, column: CHIRALPAK IH-3; column size: 4.6*50 mm, 3 μm; mobile phase: MTBE (0.1% DEA):EtOH=70:30; flow: 1.0 mL/min)).

Mass (m/z): 369 [M+H]⁺. ¹H NMR (400 MHz, CD₃OD) δ 7.87 (s, 1H), 7.85-7.78 (m, 1H), 7.58-7.48 (m, 3H), 6.90-6.83 (m, 1H), 6.61 (d, J=16.1 Hz, 1H), 6.46-6.35 (m, 1H), 5.04 (dd, J=8.6, 6.1 Hz, 1H), 4.91-4.87 (m, 1H), 4.04 (dd, J=15.9, 7.9 Hz, 1H), 3.61 (s, 3H), 3.56 (dd, J=14.8, 6.0 Hz, 1H), 2.98 (dd, J=14.8, 8.7 Hz, 1H). 312 was a yellow solid, with RT=2.064 min (CHIRAL HPLC, column: CHIRALPAK IH-3; column size: 4.6*50 mm, 3 μm; mobile phase: MTBE (0.1% DEA):EtOH=70:30; flow: 1.0 mL/min).) Mass (m/z): 369 [M+H]⁺. ¹H NMR (400 MHz, CD₃OD) δ 7.87 (s, 1H), 7.85-7.77 (m, 1H), 7.58-7.48 (m, 3H), 6.91-6.83 (m, 11H), 6.61 (d, J=16.1 Hz, 1H), 6.46-6.35 (m, 11H), 5.04 (dd, J=8.6, 6.0 Hz, 11H), 4.91-4.87 (m, 1H), 4.04 (dd, J=15.9, 7.9 Hz, 11H), 3.61 (s, 3H), 3.56 (dd, J=14.8, 6.0 Hz, 11H), 2.97 (dd, J=14.8, 8.6 Hz, 1H). The structure of 312 was confirmed by X-ray.

### (Z)-3-((3-bromopyridin-2-yl)methyl)-2-(2-fluoro-3-(1H-1,2,3-triazol-4-yl)allyl)isoindolin-1-one (314)

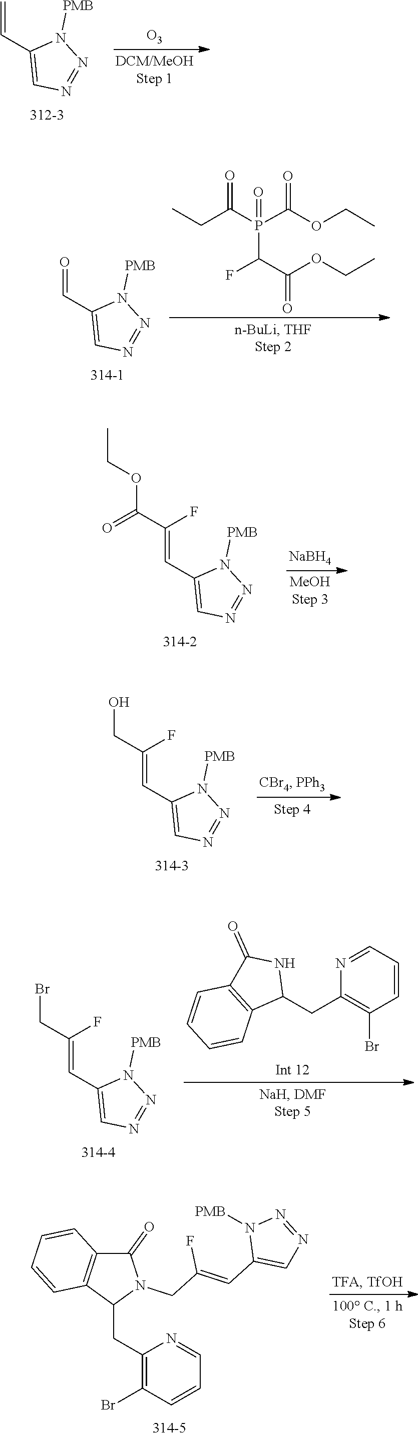

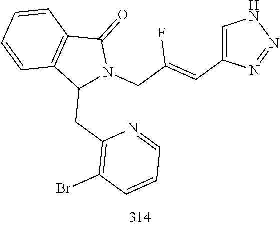 [SMILES: O=C1c2ccccc2C(Cc2ncccc2Br)N1C/C(F)=C/c1c[nH]nn1]

### Step 1. 1-(4-methoxybenzyl)-1H-1,2,3-triazole-5-carbaldehyde (314-1)

A solution of 312-3 (2.0 g, 9.29 mmol) in DCM (10 mL) and methanol (10 mL) saturated with O₃ was stirred at −78° C. until it turned to blue. N₂ was bubbled into the solution until it turned to colorless at −78° C. Then methyl sulfide (10 mL) was added and the reaction mixture was warmed to r.t. This suspension was stirred for an additional 1 h. LC-MS showed the starting material was mostly converted to the product. The reaction mixture was concentrated to give the product 1-(4-methoxybenzyl)-1H-1,2,3-triazole-5-carbaldehyde (1.80 g, 82.1%) as a yellow oil. MS (ESI) m/z 218 [M+H]⁺.

### Step 2. ethyl (Z)-2-fluoro-3-(1-(4-methoxybenzyl)-1H-1,2,3-triazol-5-yl)acrylate (314-2)

n-BuLi (0.23 mL, 3.48 mmol) was added dropwise to a solution of ethyl 2-diethoxyphosphoryl-2-fluoro-acetate (843 mg, 3.48 mmol) in THF (5 mL) at −78° C. After 30 min, a solution of 314-1 (630 mg, 2.90 mmol) in THE (5 mL) was added dropwise and the mixture was stirred at −78° C. for 1 h. The reaction was quenched with sat. NH₄Cl (aq.), then water (10 mL) was added to the mixture and the mixture was extracted with EtOAc (10 mL×3). The organics were then combined and dried (Na₂SO₄) before concentration to dryness to give the product ethyl (Z)-2-fluoro-3-(1-(4-methoxybenzyl)-1H-1,2,3-triazol-5-yl)acrylate (400 mg, 41.1%) as a yellow oil. MS (ESI) m/z 306 [M+H]⁺.

### Step 3. (Z)-2-fluoro-3-(1-(4-methoxybenzyl)-1H-1,2,3-triazol-5-yl)prop-2-en-1-ol (314-3)

To a solution of 314-2 (300 mg, 0.98 mmol) in methanol (5 mL) was added NaBH₄ (186 mg, 4.91 mmol) at r.t. under N₂. After addition, the mixture was stirred at r.t. for 1 h. LC-MS showed the starting material was mostly converted to the product. Then water (10 mL) was added to the mixture and the mixture was extracted with EtOAc (10 mL×3). The organics were then combined and dried (Na₂SO₄) before concentration to dryness to give the product (Z)-2-fluoro-3-(1-(4-methoxybenzyl)-1H-1,2,3-triazol-5-yl)prop-2-en-1-ol (200 mg, 71.1%) as a yellow oil. MS (ESI) m/z 264 [M+H]⁺.

### Step 4. (Z)-5-(3-bromo-2-fluoroprop-1-en-1-yl)-1-(4-methoxybenzyl)-1H-1,2,3-triazole (314-4)

A solution of PPh₃ (197 mg, 0.75 mmol) in dry DCM (7 mL) was added to a solution of 314-3 (180 mg, 0.68 mmol) and CBr₄ (286 mg, 0.75 mmol) in dry DCM (5 mL) at 0° C. The reaction mixture was stirred at 0° C. After the reaction was completed, H₂O (10 mL) was added to the mixture and the mixture was extracted with DCM (3×10 mL). The combined organic phase was washed with brine, dried over anhydrous Na₂SO₄, and concentrated in vacuo. The residue was purified by silica gel column chromatography (PE/EtOAc=10:1) to give the product (Z)-5-(3-bromo-2-fluoroprop-1-en-1-yl)-1-(4-methoxybenzyl)-1H-1,2,3-triazole (160 mg, 65.3%) as a yellow oil. MS (ESI) m/z 326 [M+H]⁺.

### Step 5. (Z)-3-((3-bromopyridin-2-yl)methyl)-2-(2-fluoro-3-(1-(4-methoxybenzyl)-1H-1,2,3-triazol-5-yl)allyl)isoindolin-1-one (314-5)

To a solution of Int 12 (56 mg, 0.18 mmol) in DMF (1 mL) was added NaH (9 mg, 0.22 mmol) at 0° C. under N₂. After having reacted at 0° C. for 0.5h, 314-4 (60 mg, 0.18 mmol) was added to the reaction mixture. After addition, the mixture was stirred at 0° C. for 0.5 h. LC-MS showed the starting material was mostly converted to the product. Then water (10 mL) was added to the mixture and the mixture was extracted with EtOAc (10 mL×3). The combined organic phase was washed with brine, dried over anhydrous Na₂SO₄ and concentrated in vacuo. The crude was purified by flash (PE/EtOAc=2:1) to give the product (Z)-3-((3-bromopyridin-2-yl)methyl)-2-(2-fluoro-3-(1-(4-methoxybenzyl)-1H-1,2,3-triazol-5-yl)allyl)isoindolin-1-one (80 mg, 71.4%) as a white solid. MS (ESI) m/z 548 [M+H]⁺.

### Step 6. (Z)-3-((3-bromopyridin-2-yl)methyl)-2-(2-fluoro-3-(1H-1,2,3-triazol-4-yl)allyl)isoindolin-1-one (314)

To a solution of 314-5 (110 mg, 0.20 mmol) in TFA (1.5 mL) was added TfOH (0.53 mL). After addition, the mixture was stirred at 100° C. for 1 h. LC-MS showed the starting material was mostly converted to the product. Then water (10 mL) was added to the mixture and the mixture was extracted with EtOAc (10 mL×3). The combined organic phase was washed with brine, dried over Na₂SO₄ and concentrated in vacuo. The residue was purified by prep-HPLC to give the product (Z)-3-((3-bromopyridin-2-yl)methyl)-2-(2-fluoro-3-(1H-1,2,3-triazol-5-yl)allyl)isoindolin-1-one (30 mg, 33.5%) as a white solid. MS (ESI) m/z 428 [M+H]⁺. ¹H NMR (400 MHz, DMSO-d₆) δ 8.57 (dd, J=4.6, 1.5 Hz, 1H), 8.06 (dd, J=8.1, 1.5 Hz, 1H), 7.91 (d, J=2.5 Hz, 1H), 7.74-7.70 (m, 1H), 7.56-7.45 (m, 2H), 7.30 (dd, J=8.1, 4.6 Hz, 1H), 7.08-7.01 (m, 1H), 6.06 (d, J=39.7 Hz, 1H), 5.33 (dd, J=7.5, 5.5 Hz, 1H), 4.83 (dd, J=16.2, 10.2 Hz, 1H), 4.28 (dd, J=21.1, 16.2 Hz, 1H), 3.75 (dd, J=15.0, 5.5 Hz, 1H), 3.26 (dd, J=15.0, 7.5 Hz, 1H).

### 5-((2-((1H-benzo[d][1,2,3]triazol-5-yl)methyl)-3-oxoisoindolin-1-yl)methyl)-1-methyl-1H-imidazole-4-carbonitrile (315)

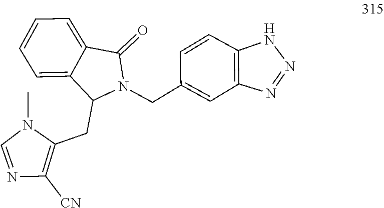 [SMILES: Cn1cnc(C#N)c1CC1c2ccccc2C(=O)N1Cc1ccc2[nH]nnc2c1]

The title compound 315 was prepared according to the procedure described for compound 279 as a white solid (3 mg, 8.1%). MS (ESI) m/z 384 [M+H]⁺. ¹H NMR (400 MHz, DMSO-d₆) δ 7.94-7.79 (m, 2H), 7.78-7.71 (m, 1H), 7.67 (d, J=7.4 Hz, 1H), 7.55 (t, J=7.4 Hz, 1H), 7.47 (t, J=7.4 Hz, 1H), 7.37 (d, J=7.5 Hz, 1H), 7.28 (s, 1H), 5.24 (d, J=15.5 Hz, 1H), 4.81 (t, J=5.4 Hz, 1H), 4.67 (d, J=15.5 Hz, 1H), 3.58 (s, 3H), 3.34-3.31 (m, 1H), 3.21 (dd, J=15.0, 6.2 Hz, 1H).

The intermediate 315-5 was prepared as follows:

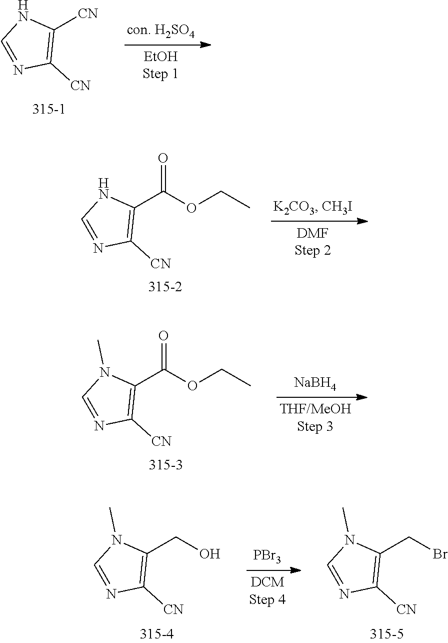

### Step 1. ethyl 4-cyano-1H-imidazole-5-carboxylate (315-2)

To a solution of 1H-imidazole-4,5-dicarbonitrile (3 g, 25.4 mmol) in EtOH (20 mL) were added con. H₂SO₄ (0.66 mL) at r.t. The mixture was refluxed for 48 h. The mixture was diluted with EtOAc and washed with water. The organic layer was separated, dried over anhydrous Na₂SO₄ and concentrated under vacuum to give 315-2 (3 g) as a light yellow solid, which was used in the next step without further purification. MS (ESI) m/z 166 [M+H]⁺.

### Step 2. ethyl 4-cyano-1-methyl-1H-imidazole-5-carboxylate (315-3)

To a solution of 315-2 (1.5 g, 9.09 mmol) in DMF (20 mL) were added K₂CO₃ (2.51 g, 18.2 mmol) and iodomethane (1.29 g, 9.09 mmol) at r.t. The mixture was stirred for 2 h at r.t. The mixture was diluted with EtOAc and washed with water. The organic layer was separated, dried over anhydrous Na₂SO₄ and purified by silica gel flash column chromatography (PE/EtOAc=1/5) to afford 315-3 (500 mg, 30.7%) as a white solid. MS (ESI) m/z 180 [M+H]⁺.

Step 3 was performed according to the procedure outlined for preparation of 164-2.

Step 4 was performed according to the procedure outlined for preparation of 195-2.

### 2-((1H-benzo[d][1,2,3]triazol-5-yl)methyl)-3-((4-chloro-3-methoxy-1-methyl-1H-pyrazol-5-yl)methyl)isoindolin-1-one (316)

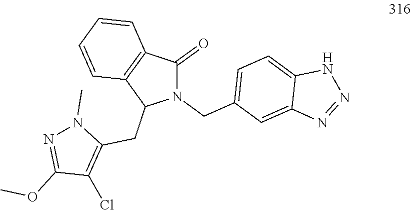 [SMILES: COc1nn(C)c(CC2c3ccccc3C(=O)N2Cc2ccc3[nH]nnc3c2)c1Cl]

The title compound 316 was prepared according to the procedure described for compound 279 as a white solid (15 mg, 27%). MS (ESI) m/z 423 [M+H]⁺. ¹H NMR (400 MHz, CD₃OD) δ 7.79-7.74 (m, 2H), 7.66 (s, 1H), 7.47-7.42 (m, 2H), 7.25 (dd, J=8.6, 1.5 Hz, 1H), 6.85-6.80 (m, 1H), 5.42 (d, J=15.4 Hz, 1H), 4.64 (dd, J=8.3, 6.2 Hz, 1H), 4.44 (d, J=15.4 Hz, 1H), 3.80 (s, 3H), 3.35 (dd, J=14.9, 6.2 Hz, 1H), 3.26 (s, 3H), 2.82 (dd, J=14.8, 8.4 Hz, 1H).

### 5-((2-((1H-benzo[d][1,2,3]triazol-5-yl)methyl)-3-oxoisoindolin-1-yl)methyl)-1-methyl-1H-pyrazole-4-carbonitrile (317)

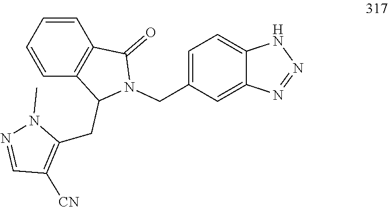 [SMILES: Cn1ncc(C#N)c1CC1c2ccccc2C(=O)N1Cc1ccc2[nH]nnc2c1]

The title compound 317 was prepared according to the procedure described for compound 279 as a white solid (10 mg, 20%). MS (ESI) m/z 384 [M+H]⁺. ¹H NMR (400 MHz, DMSO-d₆) δ 15.63 (s, 1H), 8.00-7.80 (m, 2H), 7.80-7.65 (m, 2H), 7.59-7.45 (m, 2H), 7.37-7.27 (m, 1H), 7.15-7.05 (m, 1H), 5.38 (d, J=15.4 Hz, 1H), 4.90-4.50 (m, 2H), 4.01-3.41 (m, 5H).

The intermediate 317-4 was prepared as follows:

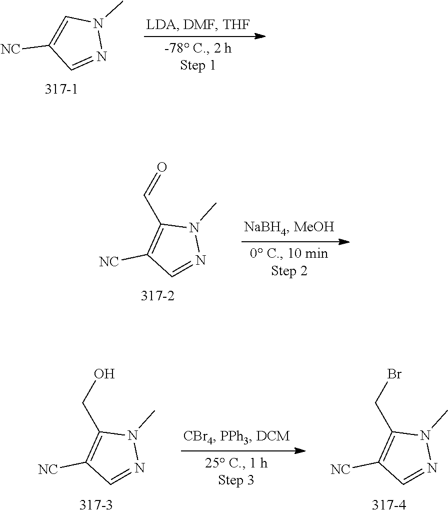

### Step 1: 5-formyl-1-methyl-1H-pyrazole-4-carbonitrile (317-2)

To a solution of 317-1 (2 g, 18.69 mmol) in THF (10 mL) was added LDA (11 mL, 22 mmol) at −78 T under N₂. After 1 h, DMF (1.63 g, 22.42 mmol) was added. The resulting mixture was stirred for additional 1 h at −78° C. under N₂. Then the mixture was quenched with NH₄Cl (aq.), and then diluted with water (20 mL). The resulting mixture was extracted with EtOAc (3×20 mL). The combined organic layers were washed with brine (20 mL), dried over anhydrous Na₂SO₄, after filtration, the filtrate was concentrated under vacuum. The residue was purified by silica gel column chromatography, eluted with (PE/EA=1/2) to afford 317-2 (1.1 g, 24%) as a white solid. MS (ESI) m/z: 136 [M+H]⁺.

Step 2 was performed according to the procedure outlined for preparation of 164-2.

Step 3 was performed according to the procedure outlined for preparation of 164-3.

### 2-((1H-benzo[d][1,2,3]triazol-5-yl)methyl)-3-((4-chloro-1-(oxetan-3-yl)-1H-pyrazol-3-yl)methyl)isoindolin-1-one (318)

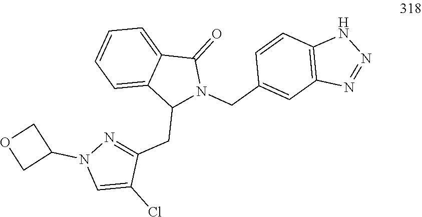 [SMILES: O=C1c2ccccc2C(Cc2nn(C3COC3)cc2Cl)N1Cc1ccc2[nH]nnc2c1]

The title compound 318 was prepared according to the procedure described for compound 279 (18 mg, 29.2%) as a white solid. MS (ESI) m/z 435 [M+H]⁺. ¹H NMR (400 MHz, DMSO-d₆) δ 15.66 (s, 1H), 7.88 (d, J=8.6 Hz, 1H), 7.78 (s, 1H), 7.77-7.70 (m, 2H), 7.55-7.46 (m, 2H), 7.26 (d, J=8.6 Hz, 1H), 6.81-6.76 (m, 1H), 5.43-5.37 (m, 1H), 5.38 (d, J=14.8 Hz, 1H), 4.70 (q, J=6.3 Hz, 2H), 4.60-4.47 (m, 3H), 4.39 (dd, J=7.7, 6.2 Hz, 1H), 3.50 (dd, J=14.9, 6.0 Hz, 1H), 3.05 (dd, J=14.9, 8.0 Hz, 1H).

The intermediate 318-3 was prepared as follows:

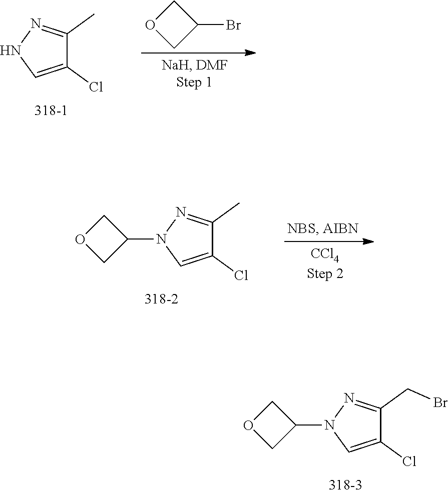

### Step 1. 4-chloro-3-methyl-1-(oxetan-3-yl)-1H-pyrazole (318-2)

To a solution of 4-chloro-3-methyl-1H-pyrazole (500 mg, 4.29 mmol) in DMF (15 mL) was added NaH (189 mg, 4.72 mmol) at 0° C.C under N₂. The mixture was stirred for 10 min. 3-bromooxetane (647 mg, 4.72 mmol) was added and the reaction mixture was warmed to r.t. This suspension was stirred for an additional 2 h. Water was added at 0° C.C and then the mixture was extracted with EtOAc. The organic layer was separated, dried over anhydrous Na₂SO₄ and concentrated to give 318-2 (650 mg) as a yellow oil, which was used in the next step without further purification. MS (ESI) m/z 173 [M+H]⁺.

Step 2 was performed according to the procedure outlined for preparation of 222-1.

### (R)-2-((1H-benzo[d][1,2,3]triazol-5-yl)methyl)-3-((4-chloro-1-methyl-1H-pyrazol-5-yl)methyl)isoindolin-1-one (319) and (S)-2-((1H-benzo[d][1,2,3]triazol-5-yl)methyl)-3-((4-chloro-1-methyl-1H-pyrazol-5-yl)methyl)isoindolin-1-one (320)

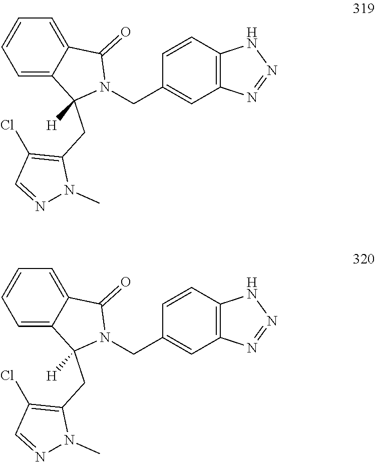 [SMILES: Cn1ncc(Cl)c1C[C@@H]1c2ccccc2C(=O)N1Cc1ccc2[nH]nnc2c1 ; Cn1ncc(Cl)c1C[C@H]1c2ccccc2C(=O)N1Cc1ccc2[nH]nnc2c1]

Chiral separation of compound 279 by chiral prep-HPLC (column: CHIRALPAK IH; column size: 2 cm*25 cm, 5 um; mobile phase A: MTBE; mobile phase B: EtOH) was conducted to afford two enantiomers: compound 319 and compound 320. One of the two enantiomers was a white solid, with RT=2.396 min (CHIRAL HPLC, column: CHIRALPAK IH-3; column size: 4.6*50 mm, 3 μm; mobile phase: MTBE (0.10% DEA):(EtOH)=80:20; flow: 1 mL/min). MS (ESI) m/z 393 [M+H]⁺. ¹H NMR (400 MHz, CDCl₃) δ 7.94 (d, J=7.0 Hz, 1H), 7.89 (d, J=8.5 Hz, 1H), 7.76 (s, 1H), 7.52-7.41 (m, 3H), 7.34 (dd, J=8.5, 1.4 Hz, 1H), 6.65 (d, J=7.3 Hz, 1H), 5.53 (d, J=15.2 Hz, 1H), 4.73 (dd, J=8.9, 6.2 Hz, 1H), 4.41 (d, J=15.2 Hz, 1H), 3.46 (s, 3H), 3.43 (dd, J=14.6, 6.2 Hz, 1H), 2.75 (dd, J=14.5, 8.9 Hz, 1H). The other one of the two enantiomers was a white solid, with RT=4.047 min (CHIRAL HPLC, column: CHIRALPAK IH-3; column size: 4.6*50 mm, 3 μm; mobile phase: MTBE (0.1% DEA):(EtOH)=80:20; flow: 1 mL/min). MS (ESI) m/z 393 [M+H]⁺. ¹H NMR (400 MHz, CDCl₃) δ 7.95 (d, J=7.3 Hz, 1H), 7.89 (d, J=8.5 Hz, 1H), 7.74 (s, 1H), 7.52-7.41 (m, 3H), 7.34 (d, J=8.4 Hz, 1H), 6.66 (d, J=7.4 Hz, 1H), 5.53 (d, J=15.1 Hz, 1H), 4.73 (dd, J=8.7, 6.1 Hz, 1H), 4.41 (d, J=15.2 Hz, 1H), 3.46 (s, 3H), 3.43 (dd, J=14.7, 6.3 Hz, 1H), 2.75 (dd, J=14.6, 8.7 Hz, 1H).

### 3-((3-bromopyridin-2-yl)methyl)-2-((6-hydroxyspiro[3.3]heptan-2-yl)methyl) isoindolin-1-one (321)

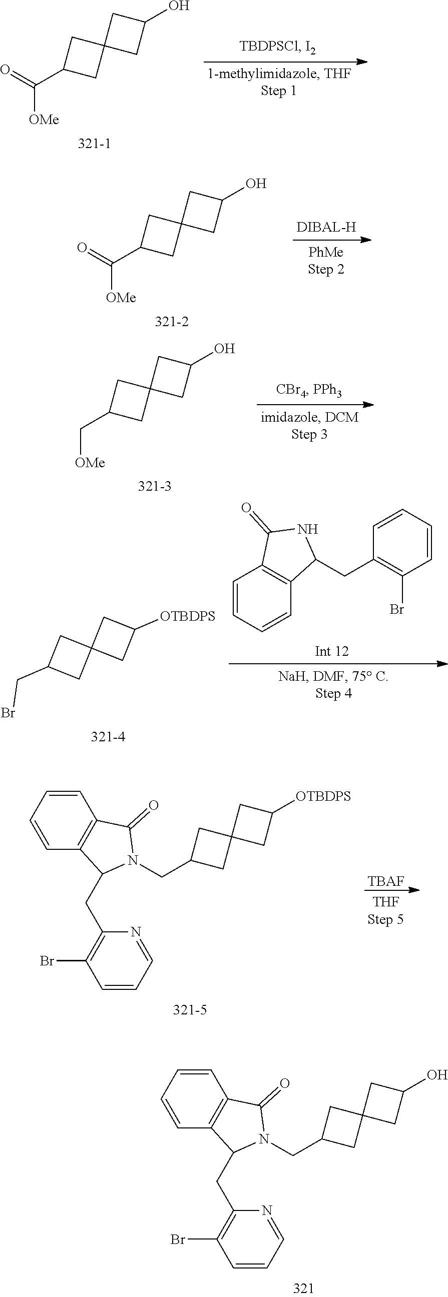

### Step 1. methyl 6-((tert-butyldiphenylsilyl)oxy)spiro[3.3]heptane-2-carboxylate (321-2)

To a solution of the methyl 2-hydroxyspiro[3.3]heptane-6-carboxylate (272 mg, 1.60 mmol), 1-methylimidazole (1.9 mL, 24.0 mmol) and I₂ (1.22 g, 4.80 mmol) in dry THF (10 mL) was added dropwise TBDPSCl (0.46 mL, 1.76 mmol) at r.t. The solution was stirred for 3 h at r.t, then the mixture was diluted with EtOAc (50 mL) and washed with 10% aqueous sodium thiosulfate (2×40 mL). Aqueous phases were then combined and extracted with EtOAc (50 mL). The organic phase was dried over MgSO₄, filtered and evaporated. The residue was purified by silica gel flash column chromatography (EtOAc/hexane=1/10) to afford 321-2 (500 mg, 76.5%) as a white solid. MS (ESI) m/z 409 [M+H]⁺.

### Step 2. [2-[tert-butyl(diphenyl)silyl]oxyspiro[3.3]heptan-6-yl]methanol (321-3)

To a solution of 321-2 (500 mg, 1.22 mmol) in toluene (6 mL) was added DIBAL-H (3.7 mL, 3.67 mmol) at −78° C. under N₂ and stirred for 1 h. Then the mixture was added water (0. 15 mL), 15% NaOH (0.15 mL) and water (0.37 mL) and then warmed up to r.t. and stirred for 15 min followed by the addition of Na₂SO₄. The mixture was filtered and washed with EtOAc (20 mL), then concentrated to afford the crude product, which was used to the next step without further purification. MS (ESI) m/z 381 [M+H]⁺.

### Step 3. [6-(bromomethyl)spiro[3.3]heptan-2-yl]oxy-tert-butyl-diphenyl-silane (321-4)

To a solution of 321-3 (200 mg, 0.53 mmol), carbon tetrabromide (192 mg, 0.58 mmol) and imidazole (71 mg, 1.05 mmol) in dry CH₂Cl₂ (2 mL) was added a solution of triphenylphosphine 20 (152 mg, 0.58 mmol) in dry CH₂Cl₂ (3 mL) at 0° C. The mixture was stirred for 2 h at 0° C., quenched with H₂O (10 mL), extracted with CH₂Cl₂ (3×30 mL), the combined organic layers was dried over Na₂SO₄, filtered and concentrated. The residue was purified by silica gel flash column chromatography (EtOAc/hexane=1/10) to yield 321-4 (150 mg, 64.4%). MS (ESI) m/z 443 [M+H]⁺.

Step 4 was performed according to the procedure outlined for preparation of Int 5.

### Step 5. 3-[(3-bromo-2-pyridyl)methyl]-2-[(2-hydroxyspiro[3.3]heptan-6-yl)methyl]isoindolin-1-one (321)

To a solution of 321-5 (100 mg, 0.15 mmol) in anhydrous THE (1 mL) was added a 1.0 M solution of TBAF (0.17 mL, 0.17 mmol). After 1 h, the solvent was removed under reduced pressure and the residue was purified by HPLC to give the product 321 (45 mg, 70.1%) as a white solid. MS (ESI) m/z 427 [M+H]⁺. ¹H NMR (400 MHz, DMSO-d₆) δ 8.61-8.59 (m, 1H), 8.10 (dd, J=8.1, 1.4 Hz, 1H), 7.67-7.63 (m, 1H), 7.51-7.41 (m, 2H), 7.31 (dd, J=8.1, 4.6 Hz, 1H), 7.02-6.94 (m, 1H), 5.19 (t, J=6.7 Hz, 1H), 4.84 (d, J=6.4 Hz, 1H), 3.94-3.83 (m, 2H), 3.57 (dd, J=14.8, 5.7 Hz, 1H), 3.19-3.07 (m, 2H), 2.45-2.33 (m, 1H), 2.28-2.22 (m, 1H), 2.19-2.11 (m, 1H), 2.05-1.79 (m, 2H), 1.76-1.67 (m, 3H), 1.62-1.57 (m, 1H).

### 3-((3-bromopyridin-2-yl)methyl)-2-((6-oxo-1,6-dihydropyridin-3-yl)methyl)isoindolin-1-one (322)

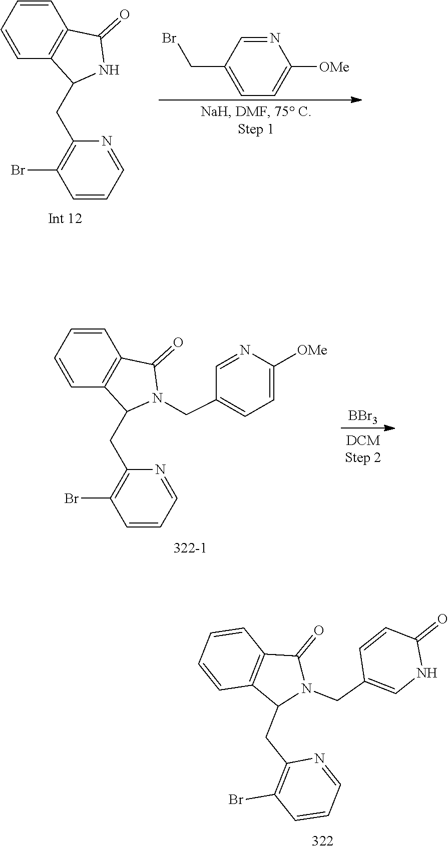

Step 1 was performed according to the procedure outlined for preparation of Int 5.

### Step 2. 3-((3-bromopyridin-2-yl)methyl)-2-((6-oxo-1,6-dihydropyridin-3-yl)methyl)isoindolin-1-one (322)

To a solution of 322-1 (95 mg, 0.22 mmol) in DCM (1 mL) was added BBr₃ (18 mg, 0.45 mmol) at 0° C. under N₂. After addition, the mixture was warmed up to r.t. and stirred for 1 h. LC-MS showed the starting material was almost converted to the product. Then water (10 mL) was added and the mixture was extracted with EtOAc (10 mL*3). The combined organics were dried (Na₂SO₄) before concentration to dryness. The residue was purified by HPLC to give 322 (70 mg, 76.2%) as a white solid. MS (ESI) m/z 410 [M+H]⁺. 1H NMR (400 MHz, DMSO-d₆) δ 11.47 (s, 1H), 8.58-8.56 (m, 1H), 8.06 (dt, J=8.2, 1.7 Hz, 1H), 7.73-7.68 (m, 1H), 7.50-7.45 (m, 2H), 7.31-7.27 (m, 1H), 7.22 (dt, J=9.5, 2.4 Hz, 1H), 7.14-7.13 (m, 1H), 7.02-6.98 (m, 1H), 6.25 (dd, J=9.5, 2.0 Hz, 1H), 5.18-5.14 (m, 1H), 4.76 (d, J=15.3 Hz, 1H), 4.20 (d, J=15.2 Hz, 1H), 3.67-3.61 (m, 1H), 3.20-3.14 (m, 11H).

### 3-((3-bromopyridin-2-yl)methyl)-2-((3-(hydroxymethyl)-[1,2,4]triazolo[4,3-a]pyridin-7-yl)methyl)isoindolin-1-one (323)

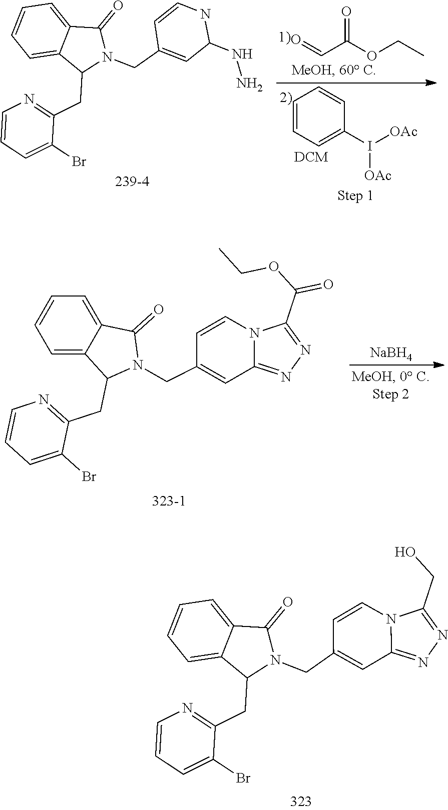

### Step 1. Ethyl 7-((1-((3-bromopyridin-2-yl)methyl)-3-oxoisoindolin-2-yl)methyl)-[1,2,4]triazolo[4,3-a]pyridine-3-carboxylate (323-1)

To a solution of 239-4 (30 mg, 0.07 mmol) in methanol (3 mL) was added ethyl 2-oxoacetate (7 mg, 0.07 mmol) and the mixture was stirred at 60 TC for 1 h. The mixture was cooled down and the solvent was removed under reduced pressure. The residue was dissolved in DCM (3 mL) and [acetoxy(phenyl)-λ3-iodanyl]acetate (23 mg, 0.07 mmol) was added. Then the mixture was stirred for 2 h at r.t. The reaction was monitored by LC/MS. Once completed, the solvent was removed under reduced pressure and the crude product was used directly in the next step.

### Step 2. 3-((3-bromopyridin-2-yl)methyl)-2-((3-(hydroxymethyl)-[1,2,4]triazolo[4,3-a]pyridin-7-yl)methyl)isoindolin-1-one (323)

To a solution of 323-1 (36 mg, 0.07 mmol) in methanol (2 mL) was added NaBH₄ (10 mg, 0.28 mmol) at 0° C. and the mixture was stirred for 2 h. The reaction was monitored by LC/MS. Once completed, the solvent was removed under reduced pressure and the residue was purified by prep-HPLC to yield 323 as a white solid (2 mg, 6%). MS (ESI) m/z 464 [M+H]⁺.

### 2-((1H-benzo[d][1,2,3]triazol-5-yl)methyl)-3-((4-chloro-1-methyl-1H-pyrazol-5-yl)methyl)-5-morpholinoisoindolin-1-one (324)

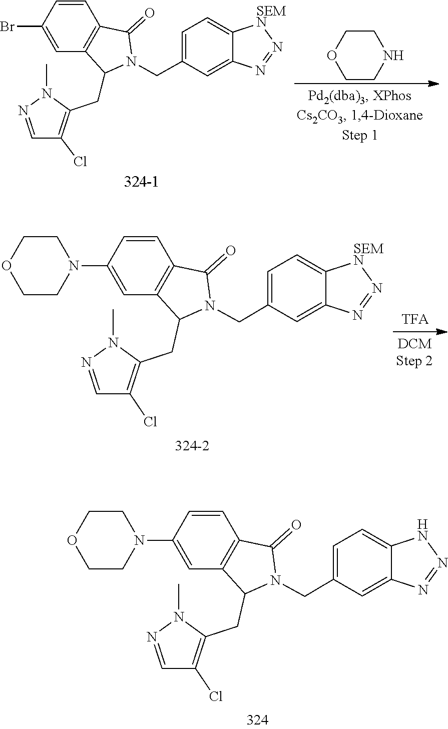 [SMILES: Cn1ncc(Cl)c1CC1c2cc(Br)ccc2C(=O)N1Cc1ccc2c(c1)nnn2COCC[Si](C)(C)C ; C1COCCN1 ; Cn1ncc(Cl)c1CC1c2cc(N3CCOCC3)ccc2C(=O)N1Cc1ccc2c(c1)nnn2COCC[Si](C)(C)C ; Cn1ncc(Cl)c1CC1c2cc(N3CCOCC3)ccc2C(=O)N1Cc1ccc2[nH]nnc2c1]

The intermediate 324-1 was prepared according to the procedure outlined for the preparation of 284-1 using Int 8 and 279-4 as starting materials.

Step 1 was performed according to the procedure outlined for preparation of 15-3.

### Step 2. 2-((1H-benzo[d][1,2,3]triazol-5-yl)methyl)-3-((4-chloro-1-methyl-1H-pyrazol-5-yl)methyl)-5-morpholinoisoindolin-1-one (324)

To a solution of 324-2 (150 mg, 0.24 mmol) in DCM (4 mL) was added TFA (4 mL). The resulting mixture was stirred at 55° C. for 1.5 h. The reaction mixture was concentrated and diluted with DCM, then adjusted to pH=7 with NaHCO₃ (aq.). The aqueous layer was separated, and the organic layer was washed with brine, dried over anhydrous Na₂SO₄ and concentrated. The residue was purified by prep HPLC to afford 324 (19 mg, 16%) as a white solid. MS (ESI) m/z 478 [M+H]H⁺. ¹H NMR (400 MHz, DMSO-d₆) δ 15.60 (s, 1H), 7.86 (d, J=8.6 Hz, 1H), 7.61 (s, 1H), 7.60-7.53 (m, 2H), 7.19 (d, J=8.6 Hz, 1H), 7.07 (dd, J=8.6, 2.2 Hz, 1H), 6.36 (d, J=2.2 Hz, 1H), 5.31 (d, J=15.2 Hz, 1H), 4.50 (t, J=6.9 Hz, 1H), 4.32 (d, J=15.3 Hz, 1H), 3.70 (t, J=4.8 Hz, 4H), 3.56 (s, 3H), 3.43 (dd, J=14.8, 6.4 Hz, 1H), 3.18-3.07 (m, 4H), 3.02 (dd, J=14.8, 7.7 Hz, 1H).

### 2-((1H-benzo[d][1,2,3]triazol-5-yl)methyl)-3-((4-chloro-1-methyl-1H-pyrazol-5-yl)methyl)-5-(morpholinomethyl)isoindolin-1-one (325)

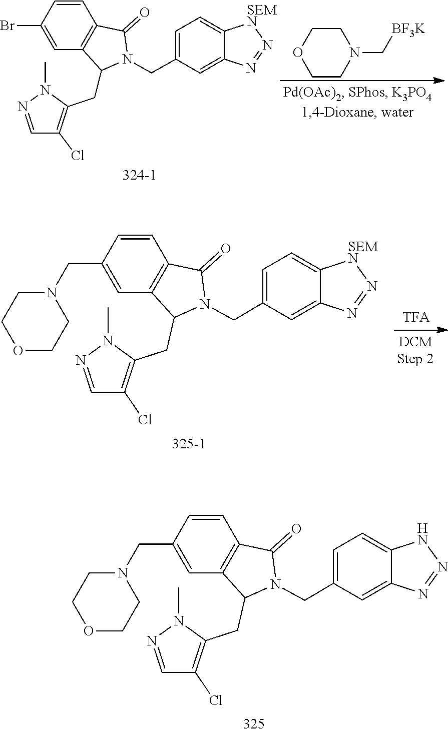

Step 1 was performed according to the procedure outlined for preparation of 96-2.

Step 2 was performed according to the procedure outlined for preparation of compound 324 to give 325 (24 mg, 19%) as a white solid. MS (ESI) m/z 492 [M+H]⁺. ¹H NMR (400 MHz, DMSO-d₆) δ 15.68 (s, 1H), 8.04-7.84 (m, 1H), 7.79-7.57 (m, 1H), 7.54 (s, 1H), 7.44 (d, J=7.8 Hz, 1H), 7.36-7.19 (m, 1H), 6.77 (s, 1H), 5.35 (d, J=15.3 Hz, 1H), 4.65 (t, J=7.0 Hz, 1H), 4.52 (d, J=15.3 Hz, 1H), 3.62-3.48 (m, 8H), 3.43 (s, 2H), 2.96 (dd, J=14.8, 8.5 Hz, 1H), 2.29 (bs, 4H).

### 2-((1H-benzo[d][1,2,3]triazol-5-yl)methyl)-5-bromo-3-((4-chloro-1-methyl-1H-pyrazol-5-yl)methyl)isoindolin-1-one (326)

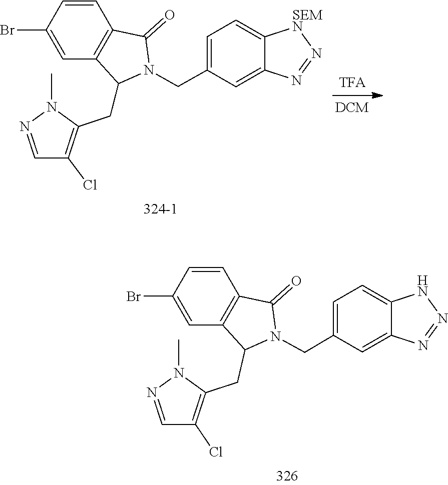 [SMILES: Cn1ncc(Cl)c1CC1c2cc(Br)ccc2C(=O)N1Cc1ccc2c(c1)nnn2COCC[Si](C)(C)C ; Cn1ncc(Cl)c1CC1c2cc(Br)ccc2C(=O)N1Cc1ccc2[nH]nnc2c1]

The title compound 326 was prepared according to the procedure described for compound 324 to give 326 (63 mg, 38%) as a white solid. MS (ESI) m/z 471 [M+H]⁺. ¹H NMR (400 MHz, DMSO-d₆) δ 15.62 (s, 1H), 7.87 (d, J=8.6 Hz, 1H), 7.75-7.65 (m, 3H), 7.55 (s, 1H), 7.24 (d, J=8.6 Hz, 1H), 7.16 (s, 1H), 5.35 (d, J=15.3 Hz, 1H), 4.69 (t, J=6.8 Hz, 1H), 4.47 (d, J=15.3 Hz, 1H), 3.60 (s, 3H), 3.51 (dd, J=14.9, 6.1 Hz, 1H), 3.10 (dd, J=15.0, 7.5 Hz, 1H).

### 2-((1H-benzo[d][1,2,3]triazol-5-yl)methyl)-3-((4-chloro-1-methyl-1H-pyrazol-5-yl)methyl)-1-oxoisoindoline-5-carbonitrile (327)

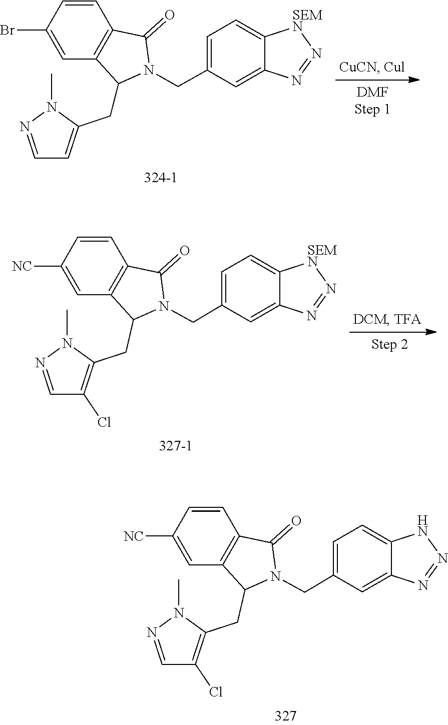 [SMILES: Cn1nccc1CC1c2cc(Br)ccc2C(=O)N1Cc1ccc2c(c1)nnn2COCC[Si](C)(C)C ; Cn1ncc(Cl)c1CC1c2cc(C#N)ccc2C(=O)N1Cc1ccc2c(c1)nnn2COCC[Si](C)(C)C ; Cn1ncc(Cl)c1CC1c2cc(C#N)ccc2C(=O)N1Cc1ccc2[nH]nnc2c1]

### Step 1. 3-((4-chloro-1-methyl-1H-pyrazol-5-yl)methyl)-1-oxo-2-((1-((2-(trimethylsilyl) ethoxy)methyl)-1H-benzo[d][1,2,3]triazol-5-yl)methyl)isoindoline-5-carbonitrile (327-1)

To a solution of 324-1 (300 mg, 0.49 mmol) in DMF (5 mL) were added CuI (19 mg, 0.09 mmol) and CuCN (134 mg, 1.50 mmol) under N₂. The mixture was stirred at 140° C. for 14 h. The mixture was diluted with EtOAc and ammonium hydroxide. The organic layer was separated, dried over anhydrous Na₂SO₄ and concentrated under vacuum. The residue was purified by silica gel column chromatography (DCM/MeOH=25/1) to give 327-1 (180 mg, 65%) as a yellow solid. MS (ESI) m/z 548 [M+H]⁺.

Step 2 was performed according to the procedure outlined for preparation of compound 324 to afford 327 (3 mg, 2.1%) as a white solid. MS (ESI) m/z 418 [M+H]⁺. ¹H NMR (400 MHz, DMSO-d₆) δ 15.51 (s, 1H), 7.99 (d, J=7.9 Hz, 1H), 7.93 (d, J=7.9 Hz, 1H), 7.86 (d, J=8.5 Hz, 1H), 7.70 (s, 1H), 7.55 (s, 1H), 7.44 (s, 1H), 7.23 (d, J=8.5 Hz, 1H), 5.38 (d, J=15.3 Hz, 1H), 4.78 (t, J=6.7 Hz, 1H), 4.50 (d, J=15.3 Hz, 1H), 3.61 (s, 3H), 3.54 (dd, J=15.1, 6.2 Hz, 1H), 3.16 (dd, J=15.0, 7.5 Hz, 1H).

### (E)-2-(3-(1H-1,2,3-triazol-4-yl)allyl)-3-((3-bromopyridin-2-yl)methyl)isoindolin-1-one (328)

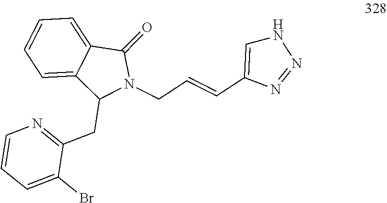 [SMILES: O=C1c2ccccc2C(Cc2ncccc2Br)N1C/C=C/c1c[nH]nn1]

The title compound 328 was prepared according to the procedure described for compound 312-5 as a white solid (8 mg, 40%). MS (ESI) m/z 410 [M+H]⁺. ¹H NMR (400 MHz, DMSO-d₆) δ 8.58 (dd, J=4.7, 1.5 Hz, 1H), 8.07 (dd, J=8.1, 1.5 Hz, 1H), 7.93 (s, 1H), 7.71 (dd, J=6.3, 2.5 Hz, 1H), 7.53-7.45 (m, 2H), 7.31 (dd, J=8.1, 4.7 Hz, 1H), 7.08-6.99 (m, 1H), 6.39-6.18 (m, 2H), 5.33 (q, J=7.2 Hz, 1H), 4.64-4.52 (m, 1H), 4.01 (dd, J=16.1, 6.1 Hz, 1H), 3.65 (dd, J=14.9, 5.9 Hz, 1H), 3.22 (dd, J=14.9, 7.4 Hz, 1H).

### 3-((3-bromopyridin-2-yl)methyl)-2-((7-fluoro-1H-benzo[d][1,2,3]triazol-5-yl)methyl) isoindolin-1-one (329)

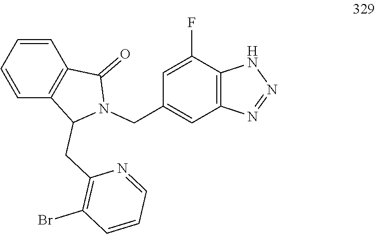 [SMILES: O=C1c2ccccc2C(Cc2ncccc2Br)N1Cc1cc(F)c2[nH]nnc2c1]

The title compound 329 was prepared according to the procedure described for compound 7 as a white solid (3 mg, 21.2%). MS (ESI) m/z 452 [M+H]⁺. ¹H NMR (400 MHz, CDCl₃) δ 8.54 (d, J=4.7 Hz, 1H), 7.95-7.89 (m, 1H), 7.81 (d, J=8.0 Hz, 1H), 7.52-7.42 (m, 2H), 7.33 (s, 1H), 7.21-7.06 (m, 2H), 6.79 (s, 1H), 5.36 (t, J=6.5 Hz, 1H), 5.19 (d, J=15.6 Hz, 1H), 4.60 (d, J=15.6 Hz, 1H), 3.52 (dd, J=15.2, 6.8 Hz, 1H), 3.30 (dd, J=15.2, 6.8 Hz, 1H).

The intermediate 329-4 was prepared as follows:

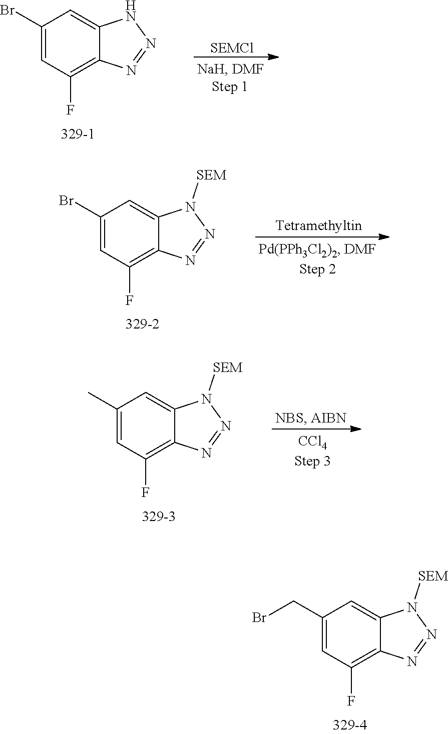

Step 1 was performed according to the procedure outlined for preparation of Int 4-2.

Step 2 was performed according to the procedure outlined for preparation of 222-5.

Step 3 was performed according to the procedure outlined for preparation of 222-1.

### 3-((3-bromopyridin-2-yl)methyl)-2-((4-fluoro-1H-benzo[d][1,2,3]triazol-5-yl)methyl)isoindolin-1-one (330)

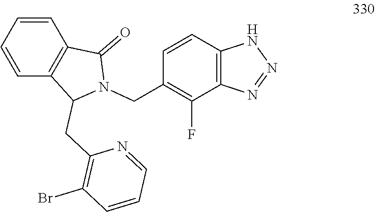 [SMILES: O=C1c2ccccc2C(Cc2ncccc2Br)N1Cc1ccc2[nH]nnc2c1F]

The title compound 330 was prepared according to the procedure described for compound 329 as a white solid (22 mg, 26.6%). MS (ESI) m/z 452 [M+H]⁺. ¹H NMR (400 MHz, CDCl₃) δ 8.50 (d, J=4.8 Hz, 1H), 7.93-7.88 (m, 1H), 7.87-7.83 (m, 1H), 7.54-7.31 (m, 4H), 7.15 (dd, J=8.0, 4.8 Hz, 1H), 7.04-6.98 (m, 1H), 5.32 (d, J=15.6 Hz, 1H), 5.26 (t, J=6.8 Hz, 1H), 4.77 (d, J=15.6 Hz, 1H), 3.64 (dd, J=15.0, 6.4 Hz, 1H), 3.30 (dd, J=15.0, 6.4 Hz, 1H).

### 2-((1H-benzo[d][1,2,3]triazol-5-yl)methyl)-3-((3-bromopyridin-2-yl)methyl)isoindolin-1-one (331)

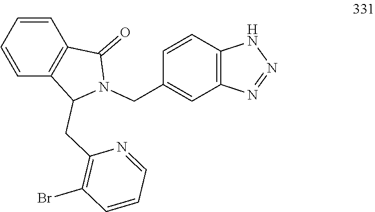 [SMILES: O=C1c2ccccc2C(Cc2ncccc2Br)N1Cc1ccc2[nH]nnc2c1]

The title compound 331 was prepared according to the procedure described for compound 7 as a white solid (9 mg, 21.2%). MS (ESI) m/z 434 [M+H]⁺. ¹H NMR (400 MHz, CDCl₃) δ 8.50 (dd, J=4.8, 1.5 Hz, 1H), 7.96-7.89 (m, 1H), 7.80 (dd, J=8.1, 1.5 Hz, 1H), 7.75 (s, 1H), 7.52 (s, 1H), 7.49-7.43 (m, 2H), 7.22-7.09 (m, 2H), 7.09-7.03 (m, 1H), 5.35-5.23 (m, 2H), 4.59 (d, J=15.5 Hz, 1H), 3.55 (dd, J=15.1, 6.6 Hz, 1H), 3.29 (dd, J=15.1, 6.4 Hz, 1H).

### 3-((3-bromopyridin-2-yl)methyl)-2-((4,5,6,7-tetrahydro-1H-benzo[d][1,2,3]triazol-5-yl)methyl)isoindolin-1-one (332)

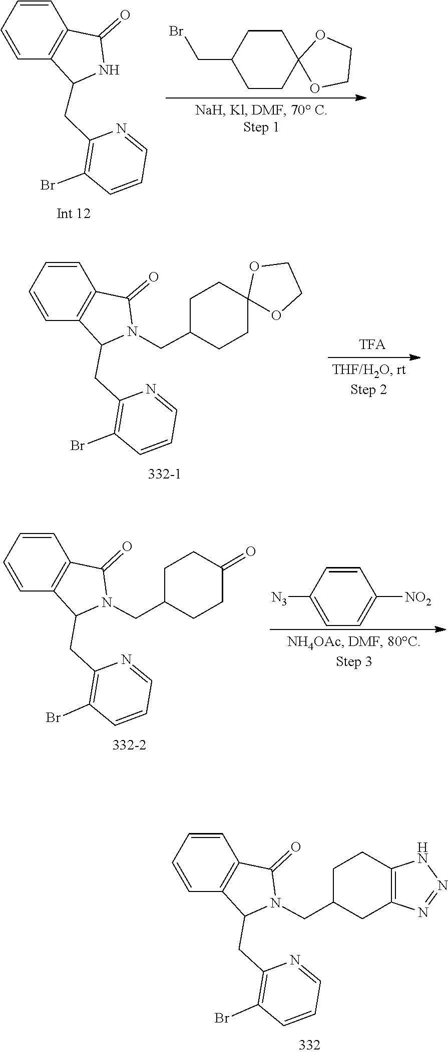 [SMILES: O=C1NC(Cc2ncccc2Br)c2ccccc21 ; BrCC1CCC2(CC1)OCCO2 ; O=C1c2ccccc2C(Cc2ncccc2Br)N1CC1CCC2(CC1)OCCO2 ; O=C1CCC(CN2C(=O)c3ccccc3C2Cc2ncccc2Br)CC1 ; [N-]=[N+]=Nc1ccc([N+](=O)[O-])cc1 ; O=C1c2ccccc2C(Cc2ncccc2Br)N1CC1CCc2[nH]nnc2C1]

### Step 1. 2-((1,4-dioxaspiro[4.5]decan-8-yl)methyl)-3-((3-bromopyridin-2-yl) methyl)isoindolin-1-one (332-1)

To a solution of Int 12 (600 mg, 1.98 mmol) in DMF (15 mL) was added NaH (95 mg, 60% in mineral oil, 2.38 mmol) at 0 T and stirred at 0 TC for 30 min. Then 8-(bromomethyl)-1,4-dioxaspiro[4.5]decane (558 mg, 2.38 mmol) and KI (328 mg, 1.98 mmol) were added. The mixture was stirred at 70 TC for 16 h. The mixture was poured into ice water (20 mL) and extracted with EA (20 mL*3) and concentrated under vacuum. The residue was purified by FCC (PE/EA=1/1) to give 332-1 as a yellow oil (450 mg, 49.8%). MS (EST) m/z 457 [M+H]⁺.

### Step 2. 3-((3-bromopyridin-2-yl)methyl)-2-((4-oxocyclohexyl)methyl)isoindolin-1-one (332-2)

To a solution of 332-1 (450 mg, 0.87 mmol) in THF/H₂O (2 mL/4 mL) was added TFA (4 mL). The mixture was stirred at r.t. for 2 h. The mixture was concentrated, adjusted to pH at 9 with sat. NaHCO₃ and extracted with EA (20 mL*2), dried over anhydrous Na₂SO₄, filtered and concentrated. The residue was purified by FCC (DCM/MeOH=20/1) to give 332-2 as a yellow solid (290 mg, 80.5%). MS (ESI) m/z 413 [M+H]⁺.

### Step 3. 3-((3-bromopyridin-2-yl)methyl)-2-((4,5,6,7-tetrahydro-1H-benzo[d][1,2,3]triazol-5-yl)methyl)isoindolin-1-one (332)

To a solution of 332-2 (260 mg, 0.63 mmol) in DMF (15 mL) were added 1-azido-4-nitrobenzene (155 mg, 0.94 mmol) and NH₄₀Ac (242 mg, 3.15 mmol). The mixture was stirred at 80′C for 16 h. The mixture was poured into water (20 mL) and extracted with EA (20 mL*3). The organic phase was concentrated and the residue was purified by prep-HPLC to give 332 as a white solid (69 mg, 25%). MS (ESI) m/z 438 [M+H]⁺. ¹H NMR (400 MHz, DMSO-d₆) δ 8.61-8.57 (m, 1H), 8.11 (d, J=7.6 Hz, 1H), 7.70 (d, J=6.8 Hz, 1H), 7.52-7.49 (m, 2H), 7.33-7.29 (m, 1H), 7.14 (t, J=8.0 Hz, 1H), 5.38-8.35 (s, 1H), 3.91-3.84 (m, 1H), 3.64-3.58 (m, 1H), 3.28-3.26 (m, 2H), 3.17-3.16 (m, 1H), 2.71-2.67 (m, 1H), 2.64-2.54 (m, 1H), 2.34-2.28 (m, 1H), 2.23-2.07 (m, 1H), 1.77-1.70 (m, 1H), 1.48-1.31 (m, 1H).

### 6-((1-((3-bromopyridin-2-yl)methyl)-3-oxoisoindolin-2-yl)methyl)-5-methylbenzo[d]oxazol-2(3H)-one (333)

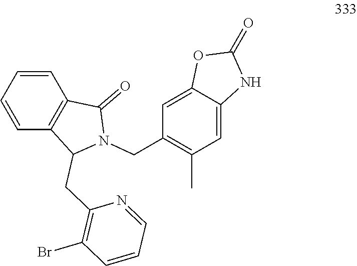 [SMILES: Cc1cc2[nH]c(=O)oc2cc1CN1C(=O)c2ccccc2C1Cc1ncccc1Br]

The title compound 333 was prepared according to general procedure F as a white solid (19 mg, 45.5%). MS (ESI) m/z 464 [M+H]⁺. ¹H NMR (400 MHz, DMSO-d₆) δ 11.39 (brs, 1H), 8.50 (dd, J=4.4, 1.2 Hz, 1H), 7.99 (dd, J=8.0, 1.2 Hz, 1H), 7.75-7.72 (m, 1H), 7.51-7.45 (m, 2H), 7.26-7.22 (m, 1H), 6.99-6.97 (m, 1H), 6.93 (s, 1H), 6.87 (s, 1H), 5.11 (t, J=6.4 Hz, 1H), 5.03 (d, J=15.6 Hz, 1H), 4.45 (d, J=16.0 Hz, 1H), 3.62 (dd, J=15.2, 5.6 Hz, 1H), 3.22-3.14 (m, 1H), 2.14 (s, 3H).

The intermediate 333-7 was prepared as follows:

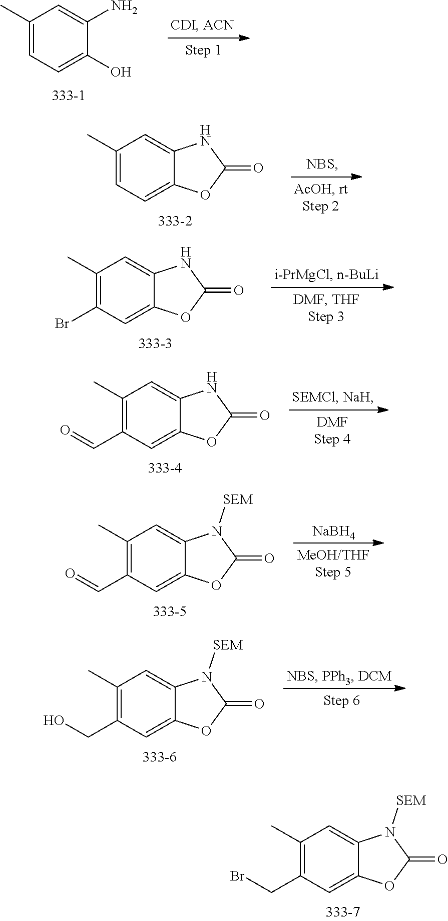

Step 1 was performed according to the procedure outlined for preparation of 223-2.

### Step 2. 6-bromo-5-methylbenzo[d]oxazol-2(3H)-one (333-3)

To a solution of 333-2 (8.9 g, 59.7 mmol) in AcOH (50 mL) was added NBS (11.7 g, 65.7 mmol). The mixture was stirred at r.t. overnight. The mixture was poured into water (50 mL) and extracted with EA (30 mL*3), dried over anhydrous Na₂SO₄, filtered and concentrated. The residue was purified by FCC (PE/EA=5/1) to give 333-3 (6.5 g, 48%) as a yellow solid. MS (ESI) m/z 228 [M+H]⁺.

### Step 3. 5-methyl-2-oxo-2,3-dihydrobenzo[d]oxazole-6-carbaldehyde (333-4)

To a solution of 333-3 (1 g, 4.37 mmol) in THF (15 mL) was added i-PrMgCl (2.4 mL, 4.8 mmol, 2 M in THF) at −10° C. and stirred at 0° C. for 1 h. n-BuLi (7 mL, 17.56 mmol, 2.5 M in hexane) was added at −40° C. and stirred at −10° C. for 30 min. DMF (3.4 mL, 43.9 mmol) was added at −10° C. and stirred at r.t. for 30 min. Aqueous citric acid was added to adjust pH=9 and extracted with EA (20 mL*3), dried over anhydrous Na₂SO₄, filtered and concentrated. The residue was purified by FCC (PE/EA=3/1) to give 333-4 as a yellow solid (570 mg, 73%). MS (ESI) m/z 178 [M+H]⁺.

Step 4 was performed according to the procedure outlined for preparation of 223-3.

### Step 5. 6-(hydroxymethyl)-5-methyl-3-((2-(trimethylsilyl)ethoxy)methyl)benzo[d]oxazol-2(3H)-one (333-6)

To a solution of 333-5 (325 mg, 1.06 mmol) in MeOH/THF (5 mL/5 mL) was added NaBH₄ (81 mg, 2.12 mmol) at 0° C. and stirred at r.t. for 1 h. The mixture was poured into ice water (20 mL) and extracted with EA (20 mL*2), dried over anhydrous Na₂SO₄, filtered and concentrated. The residue was purified by FCC (PE/EA=3/1) to give 333-6 as a colorless oil (315 mg, 96%). MS (ESI) m/z 310 [M+H]⁺.

### Step 6. 6-(bromomethyl)-5-methyl-3-((2-(trimethylsilyl)ethoxy)methyl)benzo[d]oxazol-2(3H)-one (333-7)

To a solution of 333-6 (315 mg, 1.02 mmol) and PPh₃ (320 mg, 1.22 mmol) in DCM (15 mL) was added NBS (217 mg, 1.22 mmol) at 0° C. The mixture was stirred at r.t. overnight. The mixture was poured into water (30 mL) and extracted with EA (20 mL*3), dried over anhydrous Na₂SO₄, filtered and concentrated. The residue was purified by FCC (PE/EA=5/1) to give 333-7 as a white solid (315 mg, 83%). MS (ESI) m/z 372 [M+H]⁺.

### 2-((1H-benzo[d][1,2,3]triazol-5-yl)methyl)-3-((4-chloro-1-methyl-1H-1,2,3-triazol-5-yl)methyl)isoindolin-1-one (334)

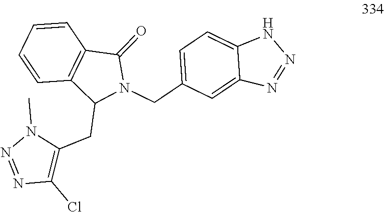 [SMILES: Cn1nnc(Cl)c1CC1c2ccccc2C(=O)N1Cc1ccc2[nH]nnc2c1]

The title compound 334 was prepared according to the procedure described for compound 279 as a white solid (65 mg, 50%). MS (ESI) m/z 394 [M+H]⁺. ¹H NMR (400 MHz, CDCl₃) δ 7.94 (d, J=6.9 Hz, 1H), 7.89 (d, J=8.6 Hz, 1H), 7.73 (s, 1H), 7.54-7.45 (m, 2H), 7.31 (d, J=8.6 Hz, 1H), 6.73 (d, J=7.3 Hz, 1H), 5.51 (d, J=15.4 Hz, 1H), 4.74 (dd, J=8.3, 6.1 Hz, 1H), 4.44 (d, J=15.4 Hz, 1H), 3.61 (s, 3H), 3.41 (dd, J=14.9, 6.0 Hz, 1H), 2.86 (dd, J=14.9, 8.4 Hz, 1H).

The intermediate 334-5 was prepared as follows:

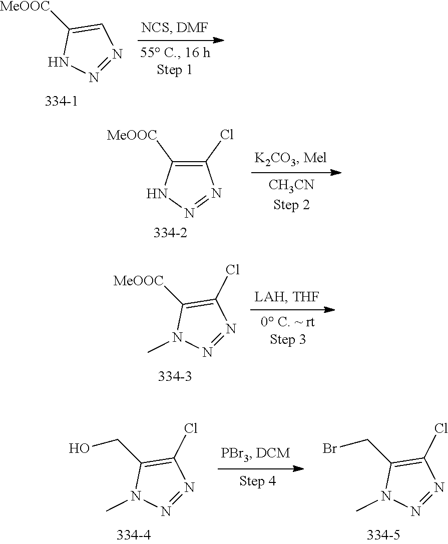

### Step 1: methyl 4-chloro-1H-1,2,3-triazole-5-carboxylate (334-2)

A mixture of methyl 1H-1,2,3-triazole-5-carboxylate (3.0 g, 24 mmol) and NCS (3.8 g, 28 mmol) in dried DMF (30 mL) was stirred at 55° C. for 16 h. The mixture was extracted with ethyl acetate (20 mL*3). The combined organic layer was washed with water (20 mL) and brine (20 mL), dried over Na₂SO₄, filtered, and concentrated in vacuo. The residue was purified by column chromatography on silica gel eluting (PE/EA=3/1 to 1/1) to give 334-2 (2.5 g, 66%) as a white solid. MS (ESI) m/z 162 [M+H]⁺.

### Step 2: methyl 4-chloro-1-methyl-1H-1,2,3-triazole-5-carboxylate (334-3)

A mixture of 334-2 (2.5 g, 16 mmol), potassium carbonate (4.4 g, 32 mmol) and iodomethane (6.8 g, 48 mmol) in acetonitrile (30 mL) was stirred at 55° C. for 16 h. The mixture was diluted with water (30 mL) and extracted with ethyl acetate (20 mL*2), The combined organic layer was washed with water (10 mL) and brine (10 mL), dried over Na₂SO₄, filtered, and concentrated in vacuo. The residue was purified by column chromatography on silica gel eluting (PE/EA=3/1 to 1/1) to give a regioisomer mixture, which was separated by SFC to give 334-3 (320 mg, 12%) as a white solid. MS (ESI) m/z 176 [M+H]⁺.

Step 3 was performed according to the procedure outlined for preparation of 279-3.

Step 4 was performed according to the procedure outlined for preparation of 279-4.

### 2-((1H-benzo[d][1,2,3]triazol-5-yl)methyl)-3-((5-chloro-2-methyl-2H-1,2,3-triazol-4-yl)methyl)isoindolin-1-one (335)

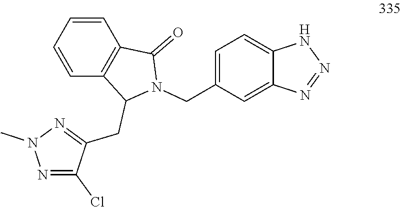 [SMILES: Cn1nc(Cl)c(CC2c3ccccc3C(=O)N2Cc2ccc3[nH]nnc3c2)n1]

The title compound 335 was prepared according to the procedure described for compound 279 as a white solid (37 mg, 47%). MS (ESI) m/z 394 [M+H]⁺. ¹H NMR (400 MHz, CDCl₃) δ 7.87-7.82 (m, 2H), 7.74 (s, 1H), 7.58-7.39 (m, 2H), 7.34-7.27 (m, 2H), 5.55 (d, J=15.6 Hz, 1H), 4.77 (t, J=5.3 Hz, 1H), 4.55 (d, J=15.6 Hz, 1H), 4.03 (s, 3H), 3.28 (dd, J=15.2, 4.5 Hz, 1H), 3.16 (dd, J=15.2, 6.1 Hz, 1H).

The intermediate 335-3 was prepared according to the procedure outlined for the preparation of 334-5.

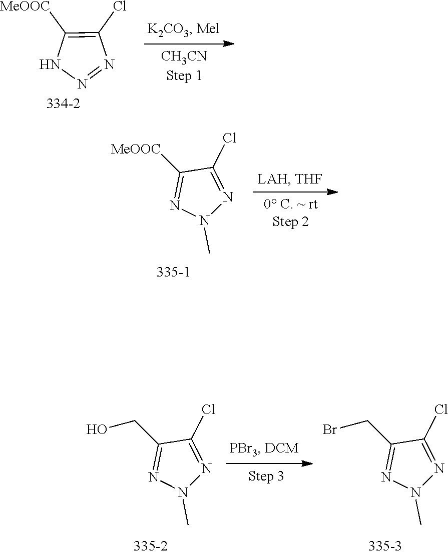

### 2-((1H-benzo[d][1,2,3]triazol-5-yl)methyl)-7-chloro-3-((4-chloro-1-methyl-1H-pyrazol-5-yl)methyl)isoindolin-1-one (336)

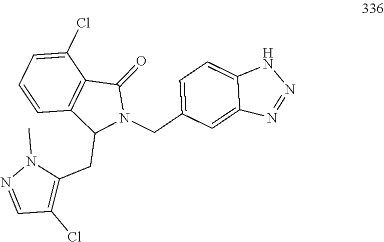 [SMILES: Cn1ncc(Cl)c1CC1c2cccc(Cl)c2C(=O)N1Cc1ccc2[nH]nnc2c1]

The title compound 336 was prepared according to the procedure described for compound 279 as a white solid (10 mg, 16%). MS (ESI) m/z 427 [M+H]⁺. ¹H NMR (400 MHz, CDCl₃) δ 7.87 (d, J=8.6 Hz, 1H), 7.73 (s, 1H), 7.50 (s, 1H), 7.43 (d, J=7.9 Hz, 1H), 7.39-7.30 (m, 2H), 6.56 (d, J=7.5 Hz, 1H), 5.50 (d, J=15.2 Hz, 1H), 4.68 (dd, J=8.6, 6.4 Hz, 1H), 4.37 (d, J=15.2 Hz, 1H), 3.48 (s, 3H), 3.40 (dd, J=14.6, 6.3 Hz, 1H), 2.74 (dd, J=14.6, 8.7 Hz, 1H).

### 2-((1H-benzo[d][1,2,3]triazol-5-yl)methyl)-3-((4-chloro-1,3-dimethyl-1H-pyrazol-5-yl)methyl)isoindolin-1-one (337)

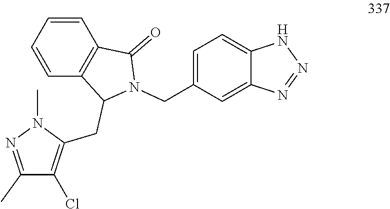 [SMILES: Cc1nn(C)c(CC2c3ccccc3C(=O)N2Cc2ccc3[nH]nnc3c2)c1Cl]

The title compound 337 was prepared according to the procedure described for compound 279 as a white solid (84 mg, 40.8%). MS (ESI) m/z 407 [M+H]⁺. ¹H NMR (400 MHz, DMSO-d₆) δ 7.87 (d, J=8.6 Hz, 1H), 7.81-7.74 (m, 1H), 7.70 (s, 1H), 7.55-7.50 (m, 2H), 7.24 (dd, J=8.6, 1.5 Hz, 1H), 6.94-6.84 (m, 1H), 5.36 (d, J=15.3 Hz, 1H), 4.64 (dd, J=8.1, 6.0 Hz, 1H), 4.47 (d, J=15.3 Hz, 1H), 3.48 (s, 3H), 3.48 (dd, J=14.8, 6.0 Hz, 1H), 2.98 (dd, J=14.8, 8.2 Hz, 1H), 2.10 (s, 3H).

### 2-((1H-benzo[d][1,2,3]triazol-5-yl)methyl)-3-((4-chloro-1-methyl-1H-pyrazol-5-yl)methyl)-4-fluoroisoindolin-1-one (338)

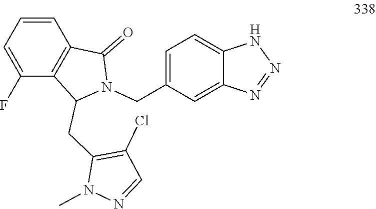 [SMILES: Cn1ncc(Cl)c1CC1c2c(F)cccc2C(=O)N1Cc1ccc2[nH]nnc2c1]

The title compound 338 was prepared according to the procedure described for compound 279 as a white solid (15 mg, 27%). MS (ESI) m/z 411 [M+H]⁺. ¹H NMR (400 MHz, CDCl₃) δ 7.86 (d, J=7.8 Hz, 1H), 7.77 (d, J=7.4 Hz, 1H), 7.56-7.51 (m, 2H), 7.28-7.21 (m, 2H), 7.08 (d, J=7.4 Hz, 1H), 5.60 (d, J=15.0 Hz, 1H), 4.75 (d, J=8.2 Hz, 1H), 4.00 (d, J=15.0 Hz, 1H), 3.61 (s, 3H), 3.44 (d, J=14.7 Hz, 1H), 3.09 (dd, J=15.0, 8.1 Hz, 1H).

### 2-((1H-benzo[d][1,2,3]triazol-5-yl)methyl)-4-chloro-3-((4-chloro-1-methyl-1H-pyrazol-5-yl)methyl)isoindolin-1-one (339)

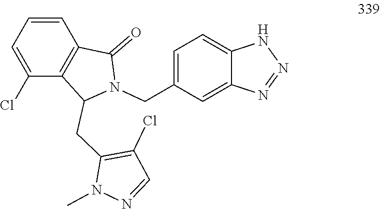 [SMILES: Cn1ncc(Cl)c1CC1c2c(Cl)cccc2C(=O)N1Cc1ccc2[nH]nnc2c1]

The title compound 339 was prepared according to the procedure described for compound 279 as a white solid (13 mg, 17%). MS (ESI) m/z 427 [M+H]⁺. ¹H NMR (400 MHz, CDCl₃) δ 7.88 (dd, J=7.2, 1.3 Hz, 1H), 7.84 (d, J=8.4 Hz, 1H), 7.61-7.50 (m, 3H), 7.13 (s, 1H), 7.02 (dd, J=8.7, 1.4 Hz, 1H), 5.62 (d, J=15.0 Hz, 1H), 4.69 (dd, J=9.6, 3.0 Hz, 1H), 3.84 (d, J=15.2 Hz, 1H), 3.84 (dd, J=1 5.3, 3.2 Hz, 1H), 3.63 (s, 3H), 2.90 (dd, J=15.4, 9.6 Hz, 1H).

### 2-((1H-benzo[d][1,2,3]triazol-5-yl)methyl)-3-((4-chloro-1-methyl-1H-pyrazol-5-yl)methyl)-6-fluoroisoindolin-1-one (340)

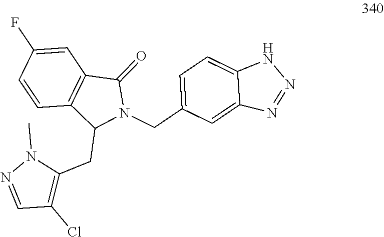 [SMILES: Cn1ncc(Cl)c1CC1c2ccc(F)cc2C(=O)N1Cc1ccc2[nH]nnc2c1]

The title compound 340 was prepared according to the procedure described for compound 279 as a white solid (15 mg, 27%). MS (ESI) m/z 411 [M+H]⁺. ¹H NMR (400 MHz, DMSO-d₆) δ 7.88 (d, J=8.5 Hz, 1H), 7.75 (s, 1H), 7.56-7.53 (m, 2H), 7.40 (td, J=9.3, 2.6 Hz, 1H), 7.28 (d, J=8.6 Hz, 1H), 6.87 (dd, J=8.4, 4.5 Hz, 1H), 5.36 (d, J=15.3 Hz, 1H), 4.67 (dd, J=8.1, 5.8 Hz, 1H), 4.53 (d, J=15.3 Hz, 1H), 3.59-3.51 (m, 1H), 3.58 (s, 3H), 3.02 (dd, J=14.9, 8.2 Hz, 1H).

### 4-((2-((1H-benzo[d][1,2,3]triazol-5-yl)methyl)-3-oxoisoindolin-1-yl)methyl)-1,3-dimethyl-1H-pyrazole-5-carbonitrile (341)

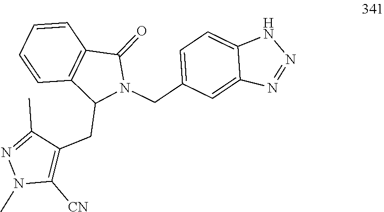 [SMILES: Cc1nn(C)c(C#N)c1CC1c2ccccc2C(=O)N1Cc1ccc2[nH]nnc2c1]

The title compound 341 was prepared according to the procedure described for compound 279 as a white solid (21 mg, 56%). MS (ESI) m/z: 398 [M+H]⁺. ¹H NMR (400 MHz, DMSO-d₆) δ 15.64 (s, 1H), 7.91-7.84 (m, 1H), 7.78-7.72 (m, 1H), 7.69 (d, J=7.3 Hz, 1H), 7.54 (t, J=7.6 Hz, 1H), 7.48 (d, J=7.2 Hz, 1H), 7.30 (d, J=7.4 Hz, 1H), 7.29-7.26 (m, 1H), 5.36 (d, J=15.4 Hz, 1H), 4.67-4.58 (m, 2H), 3.80 (s, 3H), 3.37-3.33 (m, 1H), 3.09 (d, J=14.9, 6.4 Hz, 11H), 1.9s (s, 3H).

The intermediate 341-4 was prepared according to the procedure described for preparation of 317-4.

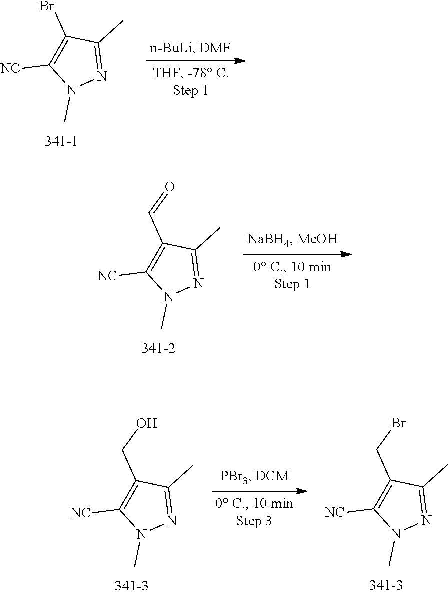

### 2-((1H-benzo[d][1,2,3]triazol-5-yl)methyl)-3-((4-chloro-1-methyl-1H-pyrazol-5-yl)methyl)-7-fluoroisoindolin-1-one (342)

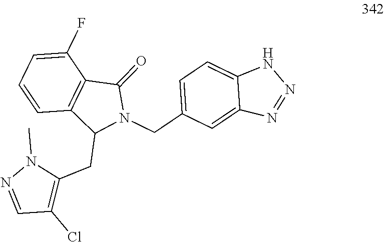 [SMILES: Cn1ncc(Cl)c1CC1c2cccc(F)c2C(=O)N1Cc1ccc2[nH]nnc2c1]

The title compound 342 was prepared according to the procedure described for compound 279 as a white solid (60 mg, 47%). MS (ESI) m/z 411 [M+H]⁺. ¹H NMR (400 MHz, DMSO-d₆) δ 15.64 (bs, 1H), 7.88 (d, J=8.5 Hz, 1H), 7.75 (s, 1H), 7.60-7.51 (m, 2H), 7.33-7.24 (m, 2H), 6.67 (d, J=7.6 Hz, 1H), 5.32 (d, J=15.3 Hz, 1H), 4.69 (dd, J=8.1, 5.9 Hz, 1H), 4.47 (d, J=15.3 Hz, 1H), 3.58-3.52 (m, 1H), 3.56 (s, 3H), 3.06 (dd, J=14.9, 8.2 Hz, 1H).

### 2-((1H-benzo[d][1,2,3]triazol-5-yl)methyl)-6-chloro-3-((4-chloro-1-methyl-1H-pyrazol-5-yl)methyl)isoindolin-1-one (343)

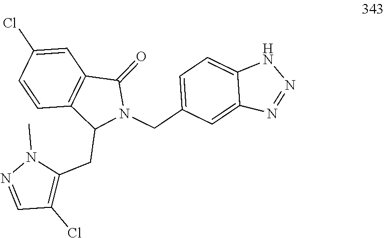 [SMILES: Cn1ncc(Cl)c1CC1c2ccc(Cl)cc2C(=O)N1Cc1ccc2[nH]nnc2c1]

The title compound 343 was prepared according to the procedure described for compound 279 as a white solid (10 mg, 16%). MS (ESI) m/z 427 [M+H]⁺. ¹H NMR (400 MHz, CDCl₃) δ 7.90 (d, J=1.6 Hz, 1H), 7.88 (d, J=8.4 Hz, 1H), 7.73 (s, 1H), 7.49 (s, 1H), 7.41 (dd, J=8.1, 2.0 Hz, 1H), 7.35 (d, J=8.5 Hz, 1H), 6.58 (d, J=8.1 Hz, 1H), 5.51 (d, J=15.2 Hz, 1H), 4.69 (dd, J=8.6, 6.6 Hz, 1H), 4.40 (d, J=15.2 Hz, 1H), 3.48 (s, 3H), 3.41 (dd, J=14.6, 6.2 Hz, 1H), 2.73 (dd, J=14.5, 9.0 Hz, 1H).

### 2-((1H-benzo[d][1,2,3]triazol-5-yl)methyl)-3-((4-chloro-1-methyl-1H-pyrazol-5-yl)methyl)-5-fluoroisoindolin-1-one (344)

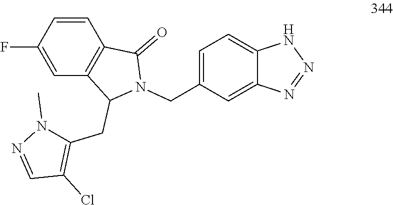 [SMILES: Cn1ncc(Cl)c1CC1c2cc(F)ccc2C(=O)N1Cc1ccc2[nH]nnc2c1]

The title compound 344 was prepared according to the procedure described for compound 279 as a white solid (6 mg, 9%). MS (ESI) m/z 411 [M+H]⁺. ¹H NMR (400 MHz, CDCl₃) b 7.93 (dd, J=8.4, 5.0 Hz, 1H), 7.87 (d, J=8.7 Hz, 1H), 7.69 (s, 1H), 7.51 (s, 1H), 7.33 (d, J=8.4 Hz, 1H), 7.20 (td, J=8.7, 2.0 Hz, 1H), 6.42 (dd, J=8.3, 2.2 Hz, 1H), 5.50 (d, J=15.2 Hz, 1H), 4.69 (dd, J=8.4, 6.6 Hz, 1H), 4.34 (d, J=15.3 Hz, 1H), 3.52 (s, 3H), 3.38 (dd, J=14.7, 6.5 Hz, 1H), 2.79 (dd, J=14.6, 8.6 Hz, 1H).

### (2s,4S)-2-(((R)-1-((3-bromopyridin-2-yl)methyl)-3-oxoisoindolin-2-yl)methyl)-5-oxa-7-azaspiro[3.4]octan-6-one (345) and (2s,4R)-2-(((S)-1-((3-bromopyridin-2-yl)methyl)-3-oxoisoindolin-2-yl)methyl)-5-oxa-7-azaspiro[3.4]octan-6-one (346)

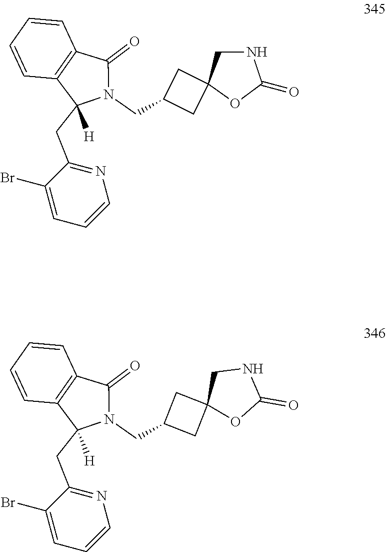 [SMILES: O=C1NC[C@]2(C[C@H](CN3C(=O)c4ccccc4[C@H]3Cc3ncccc3Br)C2)O1 ; O=C1NC[C@]2(C[C@H](CN3C(=O)c4ccccc4[C@@H]3Cc3ncccc3Br)C2)O1]

Chiral separation of compound 270 by chiral prep-HPLC (column: CHIRALPAK IF; column size: 2 cm×25 cm, 5 μm; mobile phase A: MeOH; mobile phase B: EtOH) was conducted to afford two enantiomers: compound 345 and compound 346. One of the two enantiomers was a white solid, with RT=1.286 min (CHIRAL HPLC, column: CHIRALPAK IF-3; column size: 4.6*50 mm, 3 μm; mobile phase: MTBE (0.1% DEA):EtOH=90:10; flow: 1.0 mL/min). MS (ESI) m/z 442 [M+H]⁺. ¹H NMR (400 MHz, CDCl₃) δ 8.59 (d, J=4.7 Hz, 1H), 7.91 (d, J=8.0 Hz, 1H), 7.87-7.79 (m, 1H), 7.45-7.39 (m, 2H), 7.17 (dd, J=8.1, 4.7 Hz, 1H), 6.96 (d, J=6.9 Hz, 1H), 5.60 (s, 1H), 5.30 (t, J=6.7 Hz, 1H), 4.10 (dd, J=14.5, 3.0 Hz, 1H), 3.57-3.51 (m, 3H), 3.24-3.17 (m, 2H), 2.34-2.26 (m, 5H). The other one of the two enantiomers was a white solid, with RT=2.358 min, (CHIRAL HPLC, column: CHIRALPAK IF-3; column size: 4.6*50 mm, 3 μm; mobile phase: MTBE (0.1% DEA): EtOH=90:10; flow: 1.0 mL/min). MS (ESI) m/z 442 [M+H]⁺. ¹H NMR (400 MHz, CDCl₃) δ 8.59 (dd, J=4.7, 1.5 Hz, 1H), 7.91 (dd, J=8.1, 1.5 Hz, 1H), 7.83 (dd, J=6.3, 2.3 Hz, 1H), 7.45-7.39 (m, 2H), 7.17 (dd, J=8.1, 4.7 Hz, 1H), 7.01-6.90 (m, 1H), 5.56 (s, 1H), 5.32-5.26 (m, 1H), 4.15-4.03 (m, 1H), 3.57-3.52 (m, 3H), 3.27-3.17 (m, 2H), 2.44-2.16 (m, 5H).

### 2-(3-(1H-1,2,3-triazol-4-yl)propyl)-3-((3-bromopyridin-2-yl)methyl)isoindolin-1-one (347)

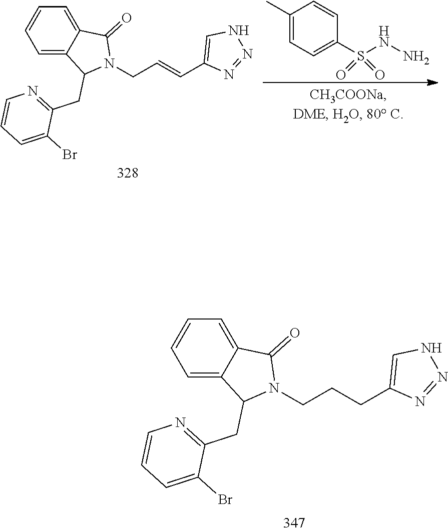 [SMILES: O=C1c2ccccc2C(Cc2ncccc2Br)N1C/C=C/c1c[nH]nn1 ; Cc1ccc(S(=O)(=O)NN)cc1 ; O=C1c2ccccc2C(Cc2ncccc2Br)N1CCCc1c[nH]nn1]

To a solution of 328 (10 mg, 0.02 mmol) in DME (1.0 mL) were added water (1 mL), sodium acetate (332 mg, 2.44 mmol) and 4-methylbenzenesulfonhydrazide (227 mg, 1.22 mmol). The mixture was stirred at 80° C. overnight. Then the mixture was diluted with water and extracted with EA. The organic layer was separated, dried over Na₂SO₄, filtered and concentrated under reduced pressure. The residue was purified by prep-HPLC to yield 347 (3 mg, 30%) as a white solid. MS (ESI) m/z 412 [M+H]⁺.

### 6-((1-((3-bromopyridin-2-yl)methyl)-3-oxoisoindolin-2-yl)methyl)quinazolin-2(1H)-one (348)

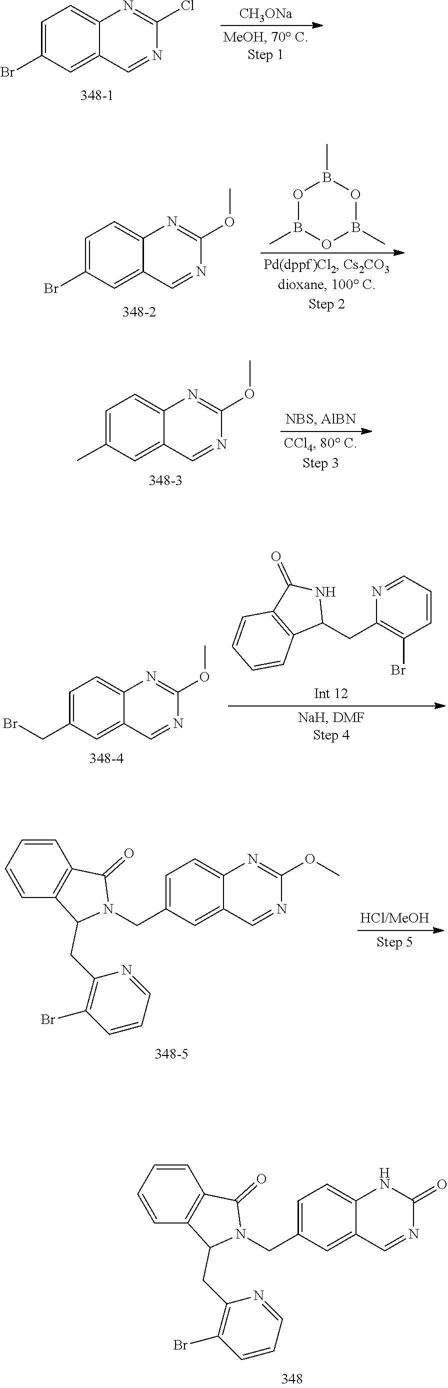

### Step 1. 6-bromo-2-methoxyquinazoline (348-2)

The mixture of 348-1 (2.6 g, 10.7 mmol) and CH₃ONa (2.9 g, 53.5 mmol) in MeOH (50 mL) was stirred at 70° C. for 1 h. The mixture was poured into ice water (20 mL) and extracted with EA (20 mL*3), dried over anhydrous Na₂SO₄, filtered and concentrated under vacuum. The 15 residue was purified by FCC (PE/EA=10/1) to give 348-2 as a white solid (2.4 g, 92%). MS (ESI) m/z 239 [M+H]⁺.

### Step 2. 2-methoxy-6-methylquinazoline (348-3)

The mixture of 348-2 (2.0 g, 8.4 mmol), 2,4,6-trimethyl-1,3,5,2,4,6-trioxatriborinane (3 mL, 10.1 mmol), Pd(dppf)Cl₂ (952 mg, 1.3 mmol) and Cs₂CO₃ (5.5 g, 16.8 mmol) in dioxane (50 mL) was 20 stirred at 100° C. for 3 h under N₂. The mixture was filtered, the filtrate was poured into water (20 mL) and extracted with EA (20 mL*3), dried over anhydrous Na₂SO₄, filtered and concentrated under vacuum. The residue was purified by FCC (PE/EA=2/1) to give 348-3 as a yellow oil (1.3 g, 87%). MS (ESI) m/z 175 [M+H]⁺.

Step 3 was performed according to the procedure outlined for preparation of 223-5.

Step 4 was performed according to the procedure outlined for preparation of 222-2.

### Step 5. 6-((1-((3-bromopyridin-2-yl)methyl)-3-oxoisoindolin-2-yl)methyl)quinazolin-2(1H)-one (348)

348-5 (100 mg, 0.21 mmol) was dissolved in HCl/MeOH (2.5 mL/10 mL), and the mixture was stirred at 80° C. for 2 h. The reaction mixture was neutralized with Na₂CO₃ and extracted with EA (20 mL*3). The combined organic phase was washed with brine, dried over anhydrous Na₂SO₄, filtered and concentrated. The residue was purified by prep-HPLC to give 348 as a white solid (15.3 mg, 16%). MS (ESI) m/z 461 [M+H]⁺. ¹H NMR (400 MHz, DMSO-d₆) δ 9.45 (s, 0.5H), 9.18 (s, 0.5H), 8.56-8.52 (m, 1H), 8.07-8.04 (m, 0.5H), 7.97 (dd, J=8.0, 1.6 Hz, 0.5H), 7.77-7.72 (m, 1H), 7.55-7.43 (m, 4H), 7.31-7.24 (m, 1H), 7.18-7.14 (m, 1H), 6.99-6.92 (m, 1.5H), 6.76-6.73 (m, 0.5H), 6.01-5.98 (m, 0.5H), 5.55-5.51 (m, 0.5H), 5.26-4.96 (m, 1H), 4.56 (d, J=15.6 Hz, 0.5H), 4.29 (t, J=15.6 Hz, 0.5H), 3.53-3.48 (m, 1H), 3.19-3.13 (m, 1H).

### 6-((1-((3-bromopyridin-2-yl)methyl)-3-oxoisoindolin-2-yl)methyl)-7-methylbenzo[d]oxazol-2(3H)-one (349)

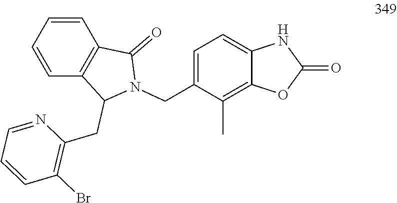 [SMILES: Cc1c(CN2C(=O)c3ccccc3C2Cc2ncccc2Br)ccc2[nH]c(=O)oc12]

The title compound 349 was prepared according to the procedure described for compound 333 as a white solid (18 mg, 40%). MS (ESI) m/z 464 [M+H]⁺. ¹H NMR (400 MHz, DMSO-d₆) δ 11.49 (s, 1H), 8.50 (dd, J=4.4, 1.2 Hz, 1H), 8.01 (dd, J=8.0, 1.6 Hz, 1H), 7.74-7.72 (m, 1H), 7.50-7.45 (m, 2H), 7.25 (dd, J=8.0, 4.4 Hz, 1H), 6.94-6.92 (m, 1H), 6.88-6.82 (m, 2H), 5.14-5.05 (m, 2H), 4.47 (d, J=16.0 Hz, 11H), 3.64 (dd, J=14.8, 5.6 Hz, 1H), 3.15 (dd, J=14.8, 7.6 Hz, 1H), 2.15 (s, 3H).

### 2-((1H-benzo[d][1,2,3]triazol-5-yl)methyl)-3-((4-chloro-1-methyl-1H-pyrazol-5-yl)methyl)-5-cyclopropylisoindolin-1-one (350)

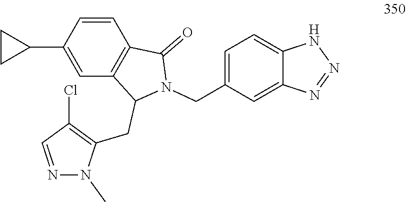 [SMILES: Cn1ncc(Cl)c1CC1c2cc(C3CC3)ccc2C(=O)N1Cc1ccc2[nH]nnc2c1]

The title compound 350 was prepared according to the procedure described for compound 279 as a white solid (25 mg, 35%). MS (ESI) m/z 433 [M+H]⁺. ¹H NMR (400 MHz, DMSO-d₆) δ 7.86 (d, J=8.6 Hz, 11H), 7.70 (s, 1H), 7.61 (d, J=7.9 Hz, 11H), 7.56 (s, 11H), 7.30 (d, J=8.1 Hz, 1H), 7.24 (d, J=8.4 Hz, 1H), 6.35 (s, 1H), 5.34 (d, J=15.2 Hz, 1H), 4.55 (dd, J=8.2, 6.0 Hz, 1H), 4.45 (d, J=15.3 Hz, 1H), 3.53 (s, 3H), 3.54-3.49 (m, 1H), 2.94 (dd, J=14.8, 8.3 Hz, 1H), 1.98-1.87 (m, 1H), 1.01-0.91 (m, 2H), 0.65-0.53 (m, 2H).

The intermediate 350-1 was prepared according to the procedure described for the preparation of 244-3.

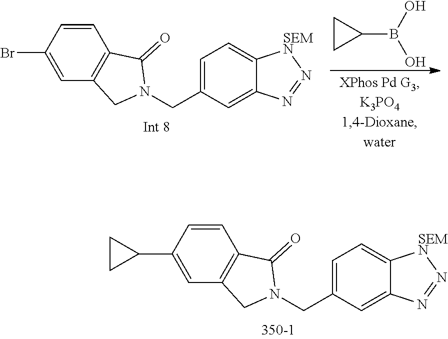

### 4-((2-((1H-benzo[d][1,2,3]triazol-5-yl)methyl)-3-oxoisoindolin-1-yl)methyl)pyridine-3,5-dicarbonitrile (351)

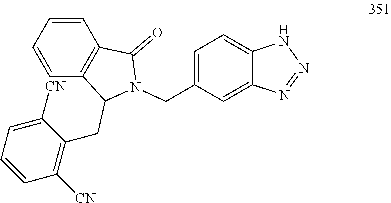 [SMILES: N#Cc1cccc(C#N)c1CC1c2ccccc2C(=O)N1Cc1ccc2[nH]nnc2c1]

The title compound 351 was prepared according to the procedure described for compound 283 as a white solid (6 mg, 12%). MS (ESI) m/z 406 [M+H]⁺. 1H NMR (400 MHz, DMSO-d₆) δ 9.24 (s, 2H), 7.86 (d, J=8.5 Hz, 1H), 7.79 (dd, J=6.6, 1.6 Hz, 1H), 7.75 (s, 1H), 7.58-7.46 (m, 2H), 7.31 (dd, J=8.7, 1.5 Hz, 1H), 6.82 (d, J=7.2 Hz, 1H), 5.29 (d, J=15.6 Hz, 1H), 4.94 (dd, J=8.5, 5.4 Hz, 11H), 4.70 (d, J=15.6 Hz, 11H), 3.90 (dd, J=13.7, 5.5 Hz, 11H), 3.05 (dd, J=13.7, 8.6 Hz, 1H).

### 4-((2-((1H-benzo[d][1,2,3]triazol-5-yl)methyl)-3-oxoisoindolin-1-yl)methyl)-5-chloro-1-methyl-1H-pyrazole-3-carbonitrile (352)

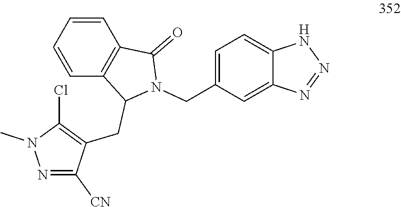 [SMILES: Cn1nc(C#N)c(CC2c3ccccc3C(=O)N2Cc2ccc3[nH]nnc3c2)c1Cl]

The title compound 352 was prepared according to the procedure described for compound 279 as a white solid (49 mg, 35.7%). MS (ESI) m/z 418 [M+H]⁺. ¹H NMR (400 MHz, DMSO-d₆) δ 15.65 (s, 1H), 7.87 (d, J=8.6 Hz, 1H), 7.78 (s, 1H), 7.66 (d, J=7.6 Hz, 1H), 7.55 (td, J=7.5, 1.3 Hz, 11H), 7.47 (t, J=7.5 Hz, 11H), 7.40 (d, J=7.5 Hz, 11H), 7.30 (dd, J=8.6, 1.5 Hz, 11H), 5.39 (d, J=15.4 Hz, 1H), 4.72-4.61 (m, 2H), 3.77 (s, 3H), 3.34 (dd, J=15.1, 4.4 Hz, 1H), 3.23 (dd, J=15.1, 5.7 Hz, 1H).

The intermediate 352-6 was prepared as follows:

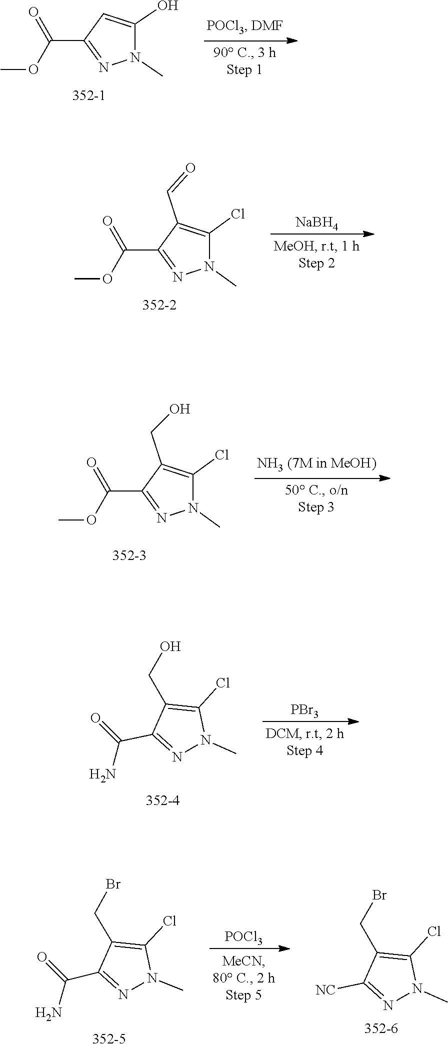

### Step 1. methyl 5-chloro-4-formyl-1-methyl-1H-pyrazole-3-carboxylate (352-2)

To an oven-dried flask was added DMF (2.8 g, 38.4 mmol). The flask was evacuated and backfilled three times with N₂. POCl₃ (14.7 g, 96.1 mmol) was added dropwise to the above flask at 0′C. The mixture was stirred at r.t. for 30 min, methyl 5-hydroxy-1-methyl-1H-pyrazole-3-carboxylate (3 g, 19.2 mmol) was added, and the reaction was heated to 90′C. This suspension was stirred for an additional 3 h. The reaction mixture was poured into water very slowly and adjusted to pH at about 7 using a sat. NaHCO₃ (aq.). The mixture was extracted with EtOAc and washed with water. The organic layer was separated, dried over anhydrous Na₂SO₄ and concentrated under vacuum to give 352-2 (1.2 g, 31%) as a yellow solid, which was used in the next step without further purification. MS (ESI) m/z 203 [M+H]⁺.

Step 2 was performed according to the procedure outlined for preparation of 164-2.

### Step 3. 5-chloro-4-(hydroxymethyl)-1-methyl-1H-pyrazole-3-carboxamide (352-4)

A solution of 352-3 (700 mg, 3.42 mmol) in NH₃ (7M in MeOH, 10 mL) was charged in a stainless steel sealed tube. The mixture was stirred at 50′C overnight. After the reaction completed, the mixture was concentrated under vacuum to afford 352-4 (650 mg) as a purple solid, which was used in the next step without further purification. MS (ESI) m/z 190 [M+H]⁺.

Step 4 was performed according to the procedure outlined for preparation of 195-2.

### Step 5. 4-(bromomethyl)-5-chloro-1-methyl-1H-pyrazole-3-carbonitrile (352-6)

To a solution of 352-5 (130 mg, 0.51 mmol) in MeCN (4 mL) was added POCl₃ (0.8 mL) under N₂. The mixture was stirred at 80° C.T for 2 h. The mixture was poured into water very slowly and adjusted to pH at about 7 using sat. NaHCO₃ (aq.). The mixture was extracted with EtOAc. The organic layer was separated, dried over anhydrous Na₂SO₄ and concentrated under vacuum. The residue was purified by Prep-TLC (PE/EtOAc=5/1) to give 352-6 (108 mg, 89.5%) as a colorless oil. MS (ESI) m/z 234 [M+H]⁺.

### 3-((2-((1H-benzo[d][1,2,3]triazol-5-yl)methyl)-3-oxoisoindolin-1-yl)methyl)pyridine-2,4-dicarbonitrile (353)

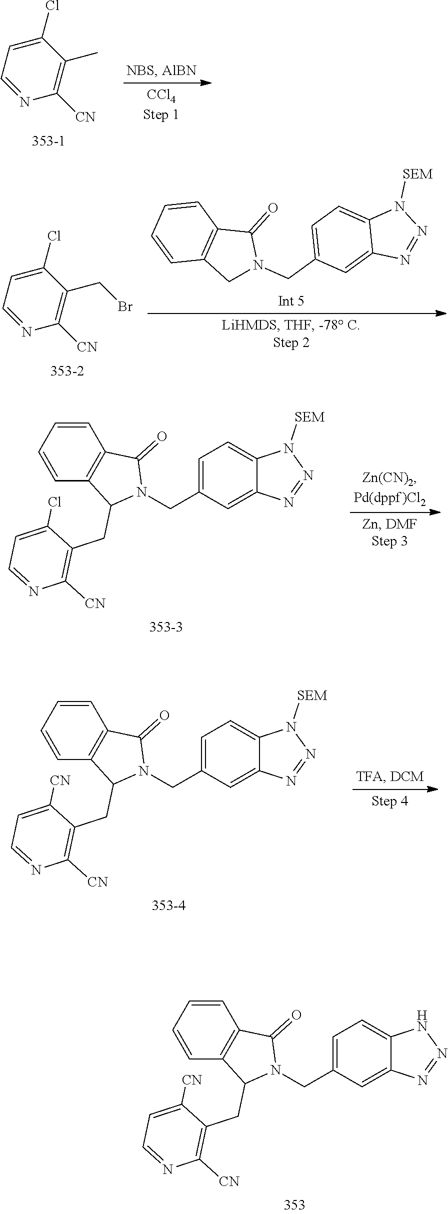

Step 1 was performed according to the procedure outlined for preparation of 222-1.

Step 2 was performed according to the procedure outlined for preparation of Int 12-2.

Step 3 was performed according to the procedure outlined for preparation of 284-2.

Step 4 was performed according to the procedure outlined for step 2 for preparation of compound 7 to afford a white solid (10 mg, 13.2%). MS (ESI) m/z 406 [M+H]⁺. ¹H NMR (400 MHz, CDCl₃) δ 8.84 d, J=5.1 Hz, 1H), 8.01-7.88 (m, 2H), 7.85 (s, 1H), 7.77-7.71 (m, 1H), 7.57-7.42 (m, 3H), 6.54 (d, J=7.8 Hz, 1H), 5.45 (d, J=15.6 Hz, 1H), 4.88 (t, J=7.1 Hz, 1H), 4.77 (d, J=15.3 Hz, 1H), 3.86-3.80 (m, 1H), 3.15-3.07 (m, 1H).

### 2-((1H-benzo[d][1,2,3]triazol-5-yl)methyl)-5-chloro-3-((4-chloro-1-methyl-1H-pyrazol-5-yl)methyl)isoindolin-1-one (354)

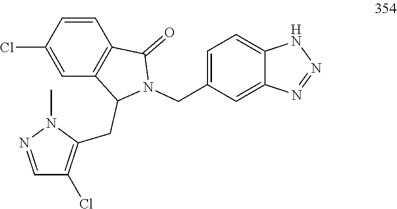 [SMILES: Cn1ncc(Cl)c1CC1c2cc(Cl)ccc2C(=O)N1Cc1ccc2[nH]nnc2c1]

The title compound 354 was prepared according to the procedure described for compound 279 as a white solid (56 mg, 26%). MS (ESI) m/z 427 [M+H]⁺. ¹H NMR (400 MHz, DMSO-d₆) δ 7.88 (d, J=8.6 Hz, 1H), 7.76 (d, J=8.1 Hz, 1H), 7.70 (s, 1H), 7.58 (d, J=8.1, 1.9 Hz, 1H), 7.55 (s, 1H), 7.24 (dd, J=8.5, 1.5 Hz, 1H), 7.02 (d, J=1.8 Hz, 1H), 5.35 (d, J=15.3 Hz, 1H), 4.70 (t, J=6.8 Hz, 1H), 4.47 (d, J=15.3 Hz, 1H), 3.61 (s, 3H), 3.52 (dd, J=14.9, 6.1 Hz, 1H), 3.11 (dd, J=14.9, 7.6 Hz, 1H).

### 2-((1H-benzo[d][1,2,3]triazol-5-yl)methyl)-3-((5-chloro-3-methyl-1H-pyrazol-1-yl)methyl)isoindolin-1-one (355)

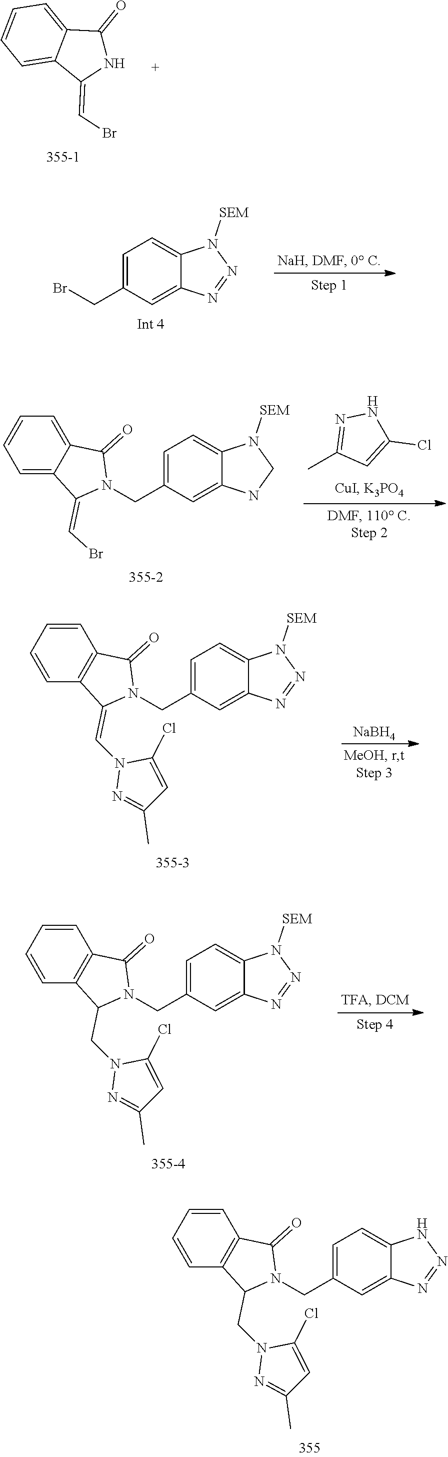

Step 1 was performed according to the procedure outlined for preparation of Int 5.

### Step 2. (Z)-3-((5-chloro-3-methyl-1H-pyrazol-1-yl)methylene)-2-((1-((2-(trimethylsilyl) ethoxy)methyl)-1H-benzo[d][1,2,3]triazol-5-yl)methyl)isoindolin-1-one (355-3)

To a solution of 355-2 (500 mg, 1.0 mmol) in dried DMF (10 mL) were added 5-chloro-3-methyl-1H-pyrazole (232 mg, 2.0 mmol), CuI (380 mg, 2.0 mmol) and K₃PO₄ (424 mg, 2.0 mmol), and the mixture was stirred at 110° C. for 9 h. The mixture was diluted with water (10 mL) and extracted with ethyl acetate (10 mL*2), The combined organic layer was washed with water (2*10 mL) and brine (2*10 mL), dried over Na₂SO₄, filtered, and concentrated in vacuo. The residue was purified by column chromatography on silica gel eluting (PE/EA=2/1 to 1/1) to give 355-3 (52 mg, 25%) as a white solid. MS (ESI) m/z 521 [M+H]⁺.

Step 3 was performed according to the procedure outlined for preparation of 164-2.

Step 4 was performed according to the procedure outlined for step 2 for the preparation of compound 7 to afford 355 as a white solid (3 mg, 7%). MS (ESI) m/z 393 [M+H]⁺. ¹H NMR (400 MHz, CDCl₃) δ 7.95 (d, J=6.4 Hz, 1H), 7.81 (d, J=8.5 Hz, 1H), 7.61 (s, 1H), 7.57-7.46 (m, 2H), 7.29-7.27 (m, 1H), 6.88 (d, J=6.8 Hz, 1H), 6.03 (s, 1H), 5.32 (d, J=15.3 Hz, 1H), 5.13 (t, J=6.9 Hz, 1H), 4.32-4.18 (m, 2H), 4.08 (dd, J=13.8, 6.6 Hz, 1H), 1.95 (s, 3H).

### 3-((2-((1H-benzo[d][1,2,3]triazol-5-yl)methyl)-4-fluoro-3-oxoisoindolin-1-yl)methyl)-4-methylpicolinonitrile (356)

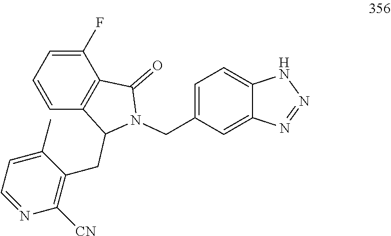 [SMILES: Cc1ccnc(C#N)c1CC1c2cccc(F)c2C(=O)N1Cc1ccc2[nH]nnc2c1]

The title compound 356 was prepared according to the procedure described for compound 284 as a white solid (30 mg, 30%). MS (ESI) m/z 413 [M+H]⁺. ¹H NMR (400 MHz, DMSO-d₆) δ 8.55 (d, J=4.9 Hz, 1H), 7.89 (d, J=8.6 Hz, 1H), 7.79 (s, 1H), 7.58 (d, J=4.9 Hz, 1H), 7.53-7.44 (m, 11H), 7.36-7.27 (m, 2H), 6.24 (d, J=7.6 Hz, 1H), 5.31 (d, J=15.5 Hz, 1H), 4.79 (dd, J=9.5, 5.9 Hz, 1H), 4.57 (d, J=15.5 Hz, 1H), 3.85 (dd, J=14.0, 5.9 Hz, 1H), 2.83 (dd, J=13.9, 9.8 Hz, 1H), 2.20 (s, 3H).

### 2-((1H-benzo[d][1,2,3]triazol-5-yl)methyl)-3-((5-chloro-3-methylisoxazol-4-yl)methyl)isoindolin-1-one (357)

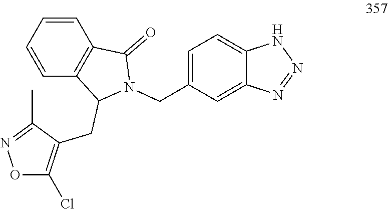 [SMILES: Cc1noc(Cl)c1CC1c2ccccc2C(=O)N1Cc1ccc2[nH]nnc2c1]

The title compound 357 was prepared according to the procedure described for compound 279 as a white solid (20 mg, 26.4%). MS (ESI) m/z 394 [M+H]⁺. ¹H NMR (400 MHz, CDCl₃) δ 7.93 (d, J=8.4 Hz, 1H), 7.87 (d, J=8.8 Hz, 1H), 7.73 (s, 1H), 7.54-7.46 (m, 2H), 7.33 (d, J=8.4 Hz, 1H), 7.04 (d, J=5.6 Hz, 1H), 5.59 (d, J=15.3 Hz, 1H), 4.55 (t, J=6.9 Hz, 1H), 4.45 (d, J=15.5 Hz, 1H), 3.09 (dd, J=14.6, 4.9 Hz, 1H), 2.59 (dd, J=14.8, 8.2 Hz, 1H), 1.95 (s, 3H).

The intermediate 357-3 was prepared as follows:

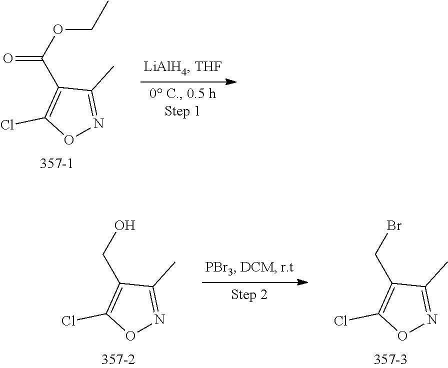

Step 1 was performed according to the procedure outlined for preparation of 263-2.

Step 2 was performed according to the procedure outlined for preparation of 263-3.

### 3-((3-bromopyridin-2-yl)methyl)-2-((2-methyl-1H-benzo[d]imidazol-5-yl)methyl)isoindolin-1-one (358)

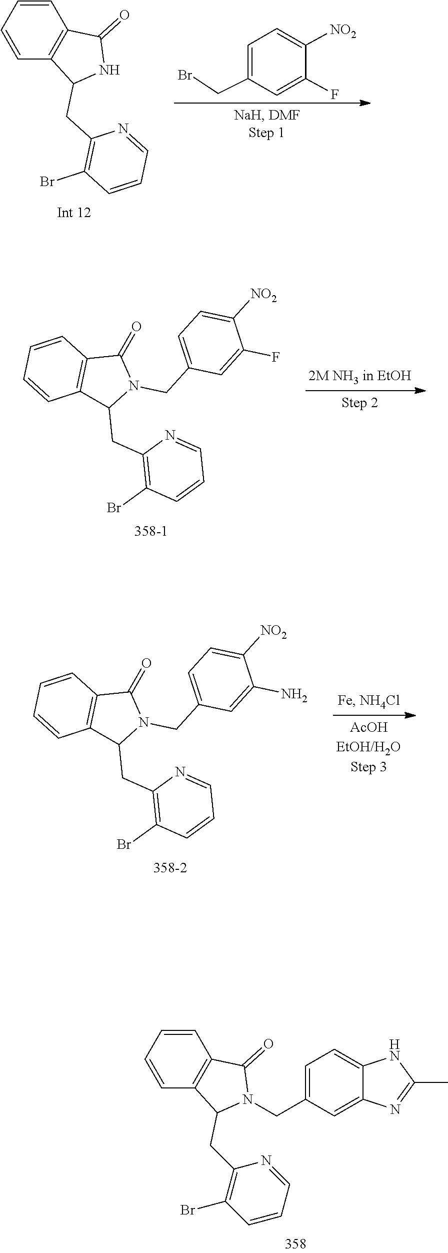 [SMILES: O=C1NC(Cc2ncccc2Br)c2ccccc21 ; O=[N+]([O-])c1ccc(CBr)cc1F ; O=C1c2ccccc2C(Cc2ncccc2Br)N1Cc1ccc([N+](=O)[O-])c(F)c1 ; Nc1cc(CN2C(=O)c3ccccc3C2Cc2ncccc2Br)ccc1[N+](=O)[O-] ; Cc1nc2cc(CN3C(=O)c4ccccc4C3Cc3ncccc3Br)ccc2[nH]1]

### Step 1. 3-((3-bromopyridin-2-yl)methyl)-2-(3-fluoro-4-nitrobenzyl)isoindolin-1-one (358-1)

To a solution of Int 12 (500 mg, 1.65 mmol) in DMF (10 mL) was added NaH (66 mg, 1.65 mmol, 60% in mineral oil) and stirred at r.t. for 20 min under N₂. Then 4-(bromomethyl)-2-fluoro-1-nitrobenzene (386 mg, 1.65 mmol) was added. The mixture was stirred at r.t. for 16 h. The mixture was quenched with water (15 mL) and extracted with EA (20 mL*2). The combined organic layers were concentrated. The residue was purified by FCC to give 358-1 as a yellow solid (200 mg, 26%). MS (ESI) m/z 456 [M+H]⁺.

### Step 2. 2-(3-amino-4-nitrobenzyl)-3-((3-bromopyridin-2-yl)methyl)isoindolin-1-one (358-2)

A mixture of 358-1 (200 mg, 0.44 mmol) in NH₃ (5 mL, 2 M in EtOH) was stirred at 80° C.T for 16 h in a sealed tube. The mixture was concentrated and purified by FCC to give 358-2 as a yellow oil (150 mg, 75%). MS (ESI) m/z 453 [M+H]⁺.

### Step 3. 3-((3-bromopyridin-2-yl)methyl)-2-((2-methyl-1H-benzo[d]imidazol-5-yl)methyl)isoindolin-1-one (358)

To a solution of 358-2 (20 mg, 0.04 mmol) in EtOH/H₂O (5 mL/0.5 mL) were added NH₄Cl (50 mg, 0.94 mmol), Fe (50 mg, 0.89 mmol) and CH₃COOH (1 drop). The mixture was stirred at 80° C. for 2 h. The mixture was filtered and the filtrate was concentrated. The residue was purified by prep-HPLC to give 358 as a white solid (2 mg, 11.5%). MS (ESI) m/z 447 [M+H]⁺. ¹H NMR (400 MHz, CD₃OD) δ 8.47 (dd, J=4.4, 1.2 Hz, 1H), 7.95 (dd, J=8.0, 1.2 Hz, 1H), 7.84-7.82 (m, 1H), 7.52-7.46 (m, 2H), 7.37 (d, J=8.0 Hz, 1H), 7.25-7.22 (m, 1H), 7.17 (s, 1H), 7.03-7.01 (m, 1H), 6.94 (d, J=8.4 Hz, 1H), 5.31 (d, J=15.2 Hz, 1H), 5.19 (t, J=6.8 Hz, 1H), 4.51 (d, J=15.2 Hz, 1H), 3.64 (dd, J=14.6, 6.4 Hz, 1H), 3.23-3.18 (m, 1H), 2.53 (s, 3H).

### 2-((1H-benzo[d][1,2,3]triazol-5-yl)methyl)-3-((2-cyano-4-methylpyridin-3-yl)methyl)-1-oxoisoindoline-5-carbonitrile (359)

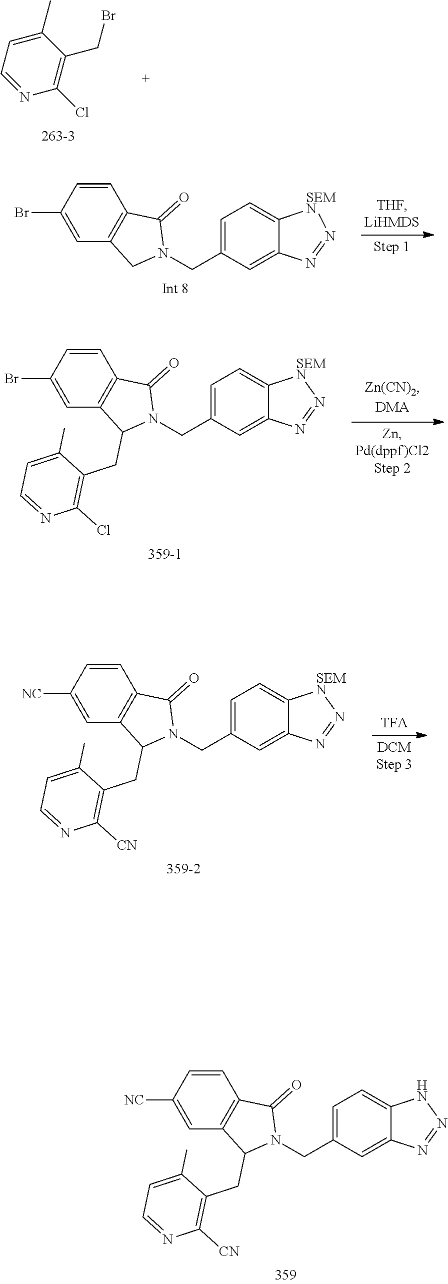

Step 1 was performed according to the procedure outlined for preparation of Int 12-2.

Step 2 was performed according to the procedure outlined for preparation of 284-2.

Step 3 was performed according to the procedure outlined for step 2 for the preparation of compound 7 to afford 359 as a white solid (2 mg, 8.5%). MS (ESI) m/z 420 [M+H]⁺. ¹H NMR (400 MHz, CD₃OD) δ 8.55 (d, J=4.9 Hz, 1H), 8.08 (d, J=7.9 Hz, 1H), 7.95 (dd, J=7.9, 1.3 Hz, 1H), 7.88 (d, J=8.6 Hz, 1H), 7.80 (s, 1H), 7.57 (d, J=5.0 Hz, 1H), 7.40 (dd, J=8.5, 1.8 Hz, 1H), 6.87 (s, 1H), 5.53 (d, J=15.5 Hz, 1H), 4.95-4.92 (m, 1H), 4.66 (d, J=15.5 Hz, 1H), 3.86 (dd, J=14.1, 6.2 Hz, 1H), 2.99 (dd, J=14.1, 9.6 Hz, 1H), 2.17 (s, 3H).

### 3-((2-((1H-benzo[d][1,2,3]triazol-5-yl)methyl)-6-cyclopropyl-3-oxoisoindolin-1-yl)methyl)-4-methylpicolinonitrile (360)

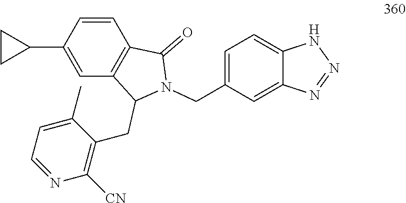 [SMILES: Cc1ccnc(C#N)c1CC1c2cc(C3CC3)ccc2C(=O)N1Cc1ccc2[nH]nnc2c1]

The title compound 360 was prepared according to the procedure described for compound 350 as a white solid (15 mg, 30%). MS (ESI) m/z 435 [M+H]⁺. ¹H NMR (400 MHz, DMSO-d₆) δ 8.57 (d, J=4.9 Hz, 1H), 7.86 (d, J=8.5 Hz, 1H), 7.74 (s, 1H), 7.66 (d, J=7.9 Hz, 1H), 7.59 (d, J=4.9 Hz, 1H), 7.33 (dd, J=7.9, 1.6 Hz, 1H), 7.26 (dd, J=8.4, 1.4 Hz, 1H), 5.88 (s, 1H), 5.33 (d, J=15.3 Hz, 1H), 4.63 (dd, J=9.6, 5.6 Hz, 1H), 4.54 (d, J=15.4 Hz, 1H), 3.83 (dd, J=13.6, 5.6 Hz, 1H), 2.76 (dd, J=13.6, 9.6 Hz, 1H), 2.18 (s, 3H), 1.93-1.81 (m, 1H), 0.99-0.82 (m, 2H), 0.52-0.42 (m, 1H), 0.42-0.32 (m, 1H).

The intermediate 360-6 was prepared as follows:

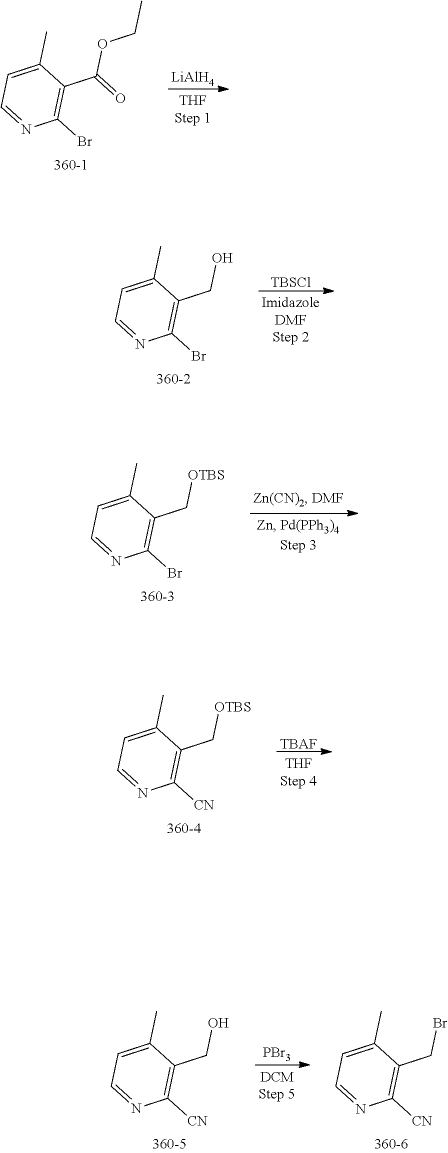

Step 1 was performed according to the procedure outlined for preparation of 263-2.

### Step 2. 2-bromo-3-(((tert-butyldimethylsilyl)oxy)methyl)-4-methylpyridine (360-3)

To a solution of 360-2 (1.4 g, 6.93 mmol) in DMF (15 mL) were added imidazole (471 mg, 6.93 mmol) and TBSCl (1.15 g, 7.62 mmol) under N₂. The mixture was stirred for 14 h, then diluted with EtOAc, and washed with water. The organic layer was separated, dried over anhydrous Na₂SO₄ and concentrated under vacuum. The residue was purified by silica gel column chromatography (PE/EtOAc=5/1) to give 360-3 (1.74 g, 79%) as a colorless oil. MS (ESI) m/z 316 [M+H]⁺.

Step 3 was performed according to the procedure outlined for preparation of 284-2.

Step 4 was performed according to the procedure outlined for step 5 for preparation of 321.

Step 5 was performed according to the procedure outlined for preparation of 263-3.

### 2-((1H-benzo[d][1,2,3]triazol-5-yl)methyl)-3-((4-chloro-3-(difluoromethyl)-1-methyl-1H-pyrazol-5-yl)methyl)isoindolin-1-one (361)

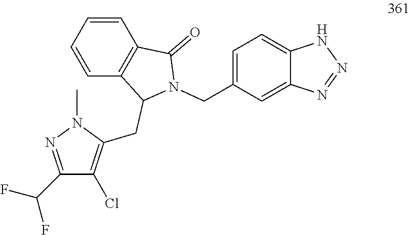 [SMILES: Cn1nc(C(F)F)c(Cl)c1CC1c2ccccc2C(=O)N1Cc1ccc2[nH]nnc2c1]

The title compound 361 was prepared according to the procedure described for compound 279 as a white solid (47 mg, 28%). MS (ESI) m/z 443 [M+H]⁺. ¹H NMR (400 MHz, DMSO-d₆) δ 15.64 (s, 1H), 7.87 (d, J=8.6 Hz, 1H), 7.82-7.72 (m, 2H), 7.58-7.48 (m, 2H), 7.27 (d, J=8.6 Hz, 1H), 6.99 (d, J=53.2 Hz, 11H), 6.91-6.85 (m, 11H), 5.37 (d, J=15.3 Hz, 1H), 4.71 (dd, J=8.2, 5.7 Hz, 1H), 4.52 (d, J=15.3 Hz, 1H), 3.61 (s, 3H), 3.60-3.52 (m, 1H), 3.06 (dd, J=15.0, 8.2 Hz, 11H).

The intermediate 361-6 was prepared as follows:

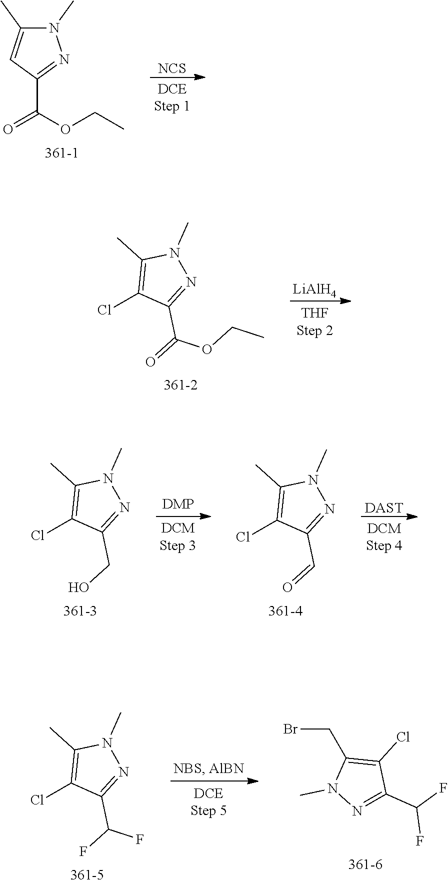

### Step 1. ethyl 4-chloro-1,5-dimethyl-1H-pyrazole-3-carboxylate (361-2)

To a solution of ethyl 1,5-dimethyl-1H-pyrazole-3-carboxylate (2 g, 11.9 mmol) in DCE (20 mL) was added NCS (3.18 g, 23.8 mmol) under N₂. The mixture was stirred at 80° C. for 14 h, then filtered. The filtrate was concentrated and purified by silica gel column chromatography (PE/EtOAc=1/1) to give 361-2 (910 mg, 37%) as a colorless oil. MS (ESI) m/z 203 [M+H]⁺.

Step 2 was performed according to the procedure outlined for preparation of 263-2.

### Step 3. 4-chloro-1,5-dimethyl-1H-pyrazole-3-carbaldehyde (361-4)

To a solution of 361-3 (650 mg, 4.05 mmol) in DCM (10 mL) was added Dess-Martin periodinane (3.43 g, 8.09 mmol) under N₂. The mixture was stirred for 2 h, the mixture was filtered and concentrated, the crude product was purified by silica gel column chromatography (PE/EtOAc=1/1) to give 361-4 (580 mg, 90%) as a yellow solid. MS (ESI) m/z 159 [M+H]⁺.

### Step 4. 4-chloro-3-(difluoromethyl)-1,5-dimethyl-1H-pyrazole (361-5)

To a solution of 361-4 (580 mg, 3.66 mmol) in DCM (10 mL) was added DAST (1.18 g, 7.31 mmol) at 0° C.T under N₂. The mixture was stirred for 14 h, then quenched with MeOH and concentrated under vacuum. The residue was purified by silica gel column chromatography (PE/EtOAc=3/1) to give 361-5 (390 mg, 59%) as a yellow oil. MS (ESI) m/z 181 [M+H]⁺.

Step 5 was performed according to the procedure outlined for preparation of 222-1.

### 2-((1H-benzo[d][1,2,3]triazol-5-yl)methyl)-3-((4-chloro-1-methyl-3-(trifluoromethyl)-1H-pyrazol-5-yl)methyl)isoindolin-1-one (362)

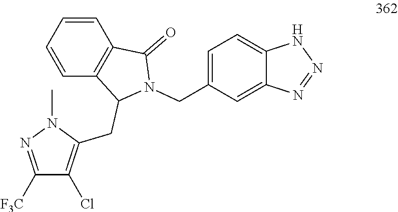 [SMILES: Cn1nc(C(F)(F)F)c(Cl)c1CC1c2ccccc2C(=O)N1Cc1ccc2[nH]nnc2c1]

The title compound 362 was prepared according to the procedure described for compound 279 as a white solid (60 mg, 36%). MS (ESI) m/z 461 [M+H]⁺. ¹H NMR (400 MHz, DMSO-d₆) δ 7.87 (d, J=8.5 Hz, 1H), 7.84-7.73 (m, 2H), 7.59-7.48 (m, 2H), 7.27 (dd, J=8.5, 1.5 Hz, 1H), 6.95-6.85 (m, 1H), 5.36 (d, J=15.2 Hz, 1H), 4.74 (dd, J=8.1, 5.6 Hz, 1H), 4.55 (d, J=15.4 Hz, 1H), 3.68 (s, 3H), 3.63 (dd, J=15.2, 5.6 Hz, 1H), 3.10 (dd, J=14.8, 8.0 Hz, 1H).

### 2-((1H-benzo[d][1,2,3]triazol-5-yl)methyl)-3-((4-chloro-1-(1,3-dihydroxypropan-2-yl)-1H-pyrazol-3-yl)methyl)isoindolin-1-one (363)

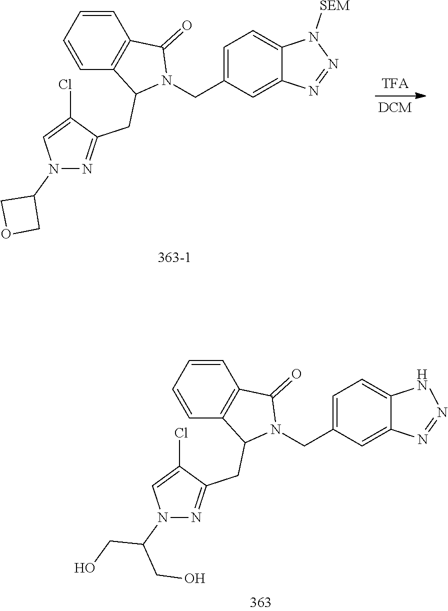 [SMILES: C[Si](C)(C)CCOCn1nnc2cc(CN3C(=O)c4ccccc4C3Cc3nn(C4COC4)cc3Cl)ccc21 ; O=C1c2ccccc2C(Cc2nn(C(CO)CO)cc2Cl)N1Cc1ccc2[nH]nnc2c1]

The intermediate 363-1 was prepared according to the procedure outlined for the preparation of compound 318.

To a solution of 363-1 (80 mg, 0.14 mmol) in DCM (2 mL) was added TFA (2 mL). The mixture was stirred at 50° C. for 2h, and then concentrated under vacuum and the residue was purified by prep-HPLC to afford 363 (15 mg, 23.3%) as a white solid. MS (ESI) m/z 453 [M+H]⁺. ¹H NMR (400 MHz, DMSO-d₆) δ 7.87 (d, J=8.5 Hz, 1H), 7.81-7.70 (m, 2H), 7.60 (s, 1H), 7.54-7.43 (m, 2H), 7.30 (d, J=8.4 Hz, 1H), 6.84 (d, J=7.1 Hz, 1H), 5.35 (d, J=15.3 Hz, 1H), 4.81 (dd, J=9.3, 5.5 Hz, 1H), 4.60 (d, J=15.3 Hz, 1H), 4.12-4.04 (m, 1H), 3.64-3.53 (m, 3H), 3.52-3.42 (m, 2H), 2.77 (dd, J=14.7, 9.3 Hz, 1H).

### 4-((2-((1H-benzo[d][1,2,3]triazol-5-yl)methyl)-3-oxoisoindolin-1-yl)methyl)-5-methylnicotinonitrile (364)

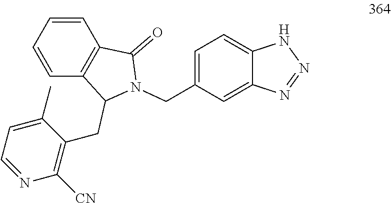 [SMILES: Cc1ccnc(C#N)c1CC1c2ccccc2C(=O)N1Cc1ccc2[nH]nnc2c1]

The title compound 364 was prepared according to the procedure described for compound 284 as a white solid (10 mg, 23%). MS (ESI) m/z 395 [M+H]⁺. ¹H NMR (400 MHz, CDCl₃) δ 8.77 (s, 1H), 8.61 (s, 1H), 7.97 (d, J=7.6 Hz, 1H), 7.90 (d, J=8.7 Hz, 1H), 7.81 (s, 1H), 7.51 (t, J=7.4 Hz, 1H), 7.43 (d, J=8.5 Hz, 1H), 7.37 (t, J=7.6 Hz, 1H), 6.44 (d, J=7.7 Hz, 1H), 5.45 (d, J=15.1 Hz, 1H), 4.83 (dd, J=9.3, 5.8 Hz, 1H), 4.62 (d, J=15.1 Hz, 1H), 3.62 (dd, J=13.1, 5.7 Hz, 1H), 2.82 (dd, J=13.0, 9.6 Hz, 1H), 1.97 (s, 3H).

The intermediate 364-3 was prepared as follows:

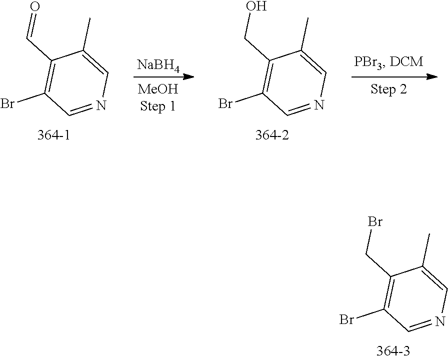

Step 1 was performed according to the procedure outlined for preparation of 164-2.

Step 2 was performed according to the procedure outlined for preparation of 263-3.

### 2-((1H-benzo[d][1,2,3]triazol-5-yl)methyl)-3-((1,4-dimethyl-1H-pyrazol-5-yl)methyl)isoindolin-1-one (365)

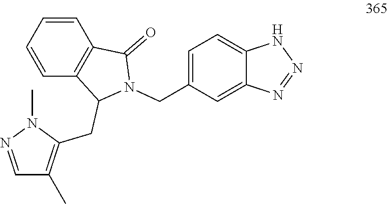 [SMILES: Cc1cnn(C)c1CC1c2ccccc2C(=O)N1Cc1ccc2[nH]nnc2c1]

The title compound 365 was prepared according to the procedure described for compound 279 as a white solid (8 mg, 27%). MS (ESI) m/z 373 [M+H]⁺. ¹H NMR (400 MHz, CDCl₃) δ 7.95 (d, J=7.4 Hz, 1H), 7.86 (s, 1H), 7.55-7.41 (m, 3H), 7.37 (s, 1H), 6.74 (d, J=7.4 Hz, 1H), 7.30-7.27 (m, 1H), 5.61 (d, J=15.2 Hz, 1H), 4.46 (t, J=7.7 Hz, 1H), 4.23 (d, J=15.2 Hz, 1H), 3.48 (s, 3H), 3.25 (dd, J=14.6, 7.1 Hz, 1H), 2.77 (dd, J=14.6, 8.2 Hz, 1H), 1.86 (s, 3H).

### 5-((1-((3-bromopyridin-2-yl)methyl)-3-oxoisoindolin-2-yl)methyl)-1H-benzo[d]imidazole-2-carbonitrile (366) and 3-((3-bromopyridin-2-yl)methyl)-2-((2-hydroxy-1H-benzo[d]imidazol-5-yl)methyl)isoindolin-1-one (367)

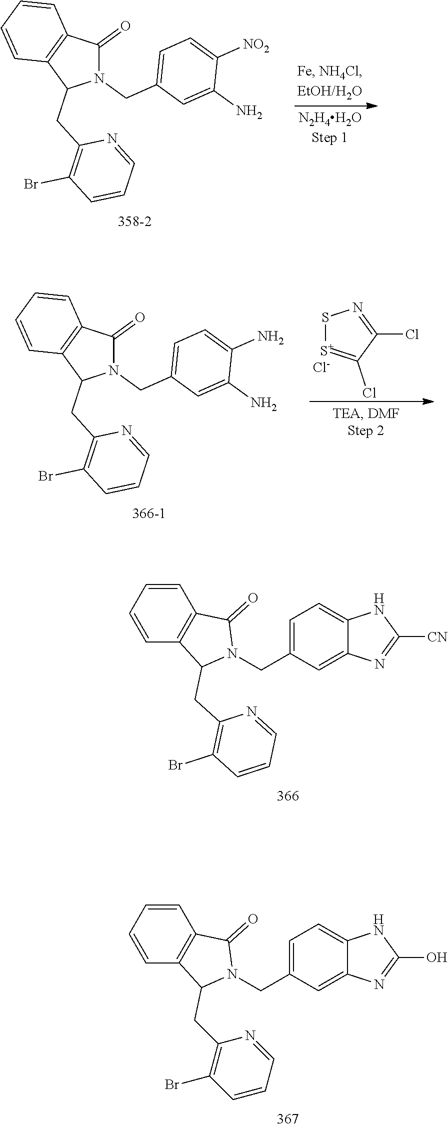 [SMILES: Nc1cc(CN2C(=O)c3ccccc3C2Cc2ncccc2Br)ccc1[N+](=O)[O-] ; Nc1ccc(CN2C(=O)c3ccccc3C2Cc2ncccc2Br)cc1N ; [Cl-] ; Clc1ns[s+]c1Cl ; N#Cc1nc2cc(CN3C(=O)c4ccccc4C3Cc3ncccc3Br)ccc2[nH]1 ; O=C1c2ccccc2C(Cc2ncccc2Br)N1Cc1ccc2[nH]c(O)nc2c1]

### Step 1. 3-((3-bromopyridin-2-yl)methyl)-2-(3,4-diaminobenzyl)isoindolin-1-one (366-1)

To a solution of 358-2 (100 mg, 0.22 mmol) in EtOH/H₂O (5 mL/0.5 mL) were added NH₄C₁ (274 mg, 5.17 mmol) and Fe (273 mg, 4.88 mmol). The mixture was stirred at r.t. for 10 min. N₂H₄ 4H₂O (5 drop) was added. The mixture was stirred at 80° C. for 2 h. The mixture was concentrated and purified by FCC to give 366-1 as a yellow solid (50 mg, 53%). MS (ESI) m/z 423 [M+H]⁺.

### Step 2. 5-((1-((3-bromopyridin-2-yl)methyl)-3-oxoisoindolin-2-yl)methyl)-1H-benzo[d]imidazole-2-carbonitrile (366) and 3-((3-bromopyridin-2-yl)methyl)-2-((2-hydroxy-1H-benzo[d]imidazol-5-yl)methyl)isoindolin-1-one (367)

To a solution of 366-1 (40 mg, 0.094 mmol) in DMF (1 mL) were added 4,5-dichloro-1,2,3-dithiazol-1-ium (38 mg, 0.19 mmol) and TEA (28 mg, 0.28 mmol) at 0° C. The mixture was stirred at r.t. for 16 h. The mixture was concentrated and purified by prep-HPLC to give compound 366 and 367. Compound 366 was a yellow solid (0.4 mg, 1.10%), MS (ESI) m/z 458 [M+H]⁺. ¹H NMR (400 MHz, CDCl₃) δ 8.53 (s, 1H), 7.86-7.84 (m, 2H), 7.57-7.44 (m, 4H), 7.16-7.05 (m, 3H), 5.35-5.24 (m, 2H), 4.61 (d, J=15.2 Hz, 1H), 3.58-3.56 (m, 1H), 3.32-3.27 (m, 1H). Compound 367 was a yellow solid (0.3 mg, 10%), MS (ESI) m/z 449 [M+H]⁺. ¹H NMR (400 MHz, CDCl₃) δ 8.54 (s, 1H), 8.09 (brs, 2H), 7.88 (t, J=7.2 Hz, 2H), 7.45 (d, J=7.2 Hz, 2H), 7.18-7.14 (m, 1H), 7.00-6.98 (m, 1H), 6.88-6.87 (m, 2H), 6.78-6.77 (m, 1H), 5.23-5.17 (m, 2H), 4.38 (d, J=15.5 Hz, 1H), 3.59-3.55 (m, 1H), 3.27-3.25 (m, 1H).

### (R)-3-((2-((1H-benzo[d][1,2,3]triazol-5-yl)methyl)-3-oxoisoindolin-1-yl)methyl)-4-methylpicolinonitrile (368) and (S)-3-((2-((1H-benzo[d][1,2,3]triazol-5-yl)methyl)-3-oxoisoindolin-1-yl)methyl)-4-methylpicolinonitrile (369)

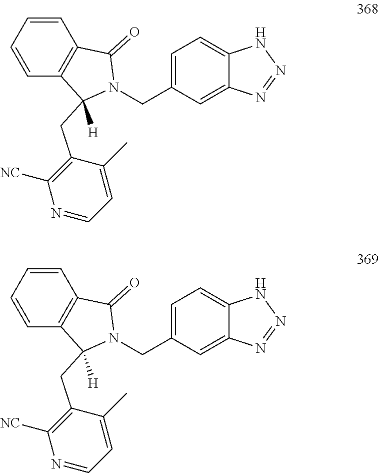 [SMILES: Cc1ccnc(C#N)c1C[C@@H]1c2ccccc2C(=O)N1Cc1ccc2[nH]nnc2c1 ; Cc1ccnc(C#N)c1C[C@H]1c2ccccc2C(=O)N1Cc1ccc2[nH]nnc2c1]

Chiral separation of compound 284 by chiral Prep-HPLC (column: CHIRALPAK IH; column size: 2 cm×25 cm, 5 μm; mobile phase A: MTBE; mobile phase B: EtOH) was conducted to afford two enantiomers: Compound 368 and Compound 369. One of the two enantiomers was a white solid, with RT=2.235 min (CHIRAL HPLC, column: CHIRALPAK IH-3; column size: 4.6*50 mm, 3 μm; mobile phase: MtBE(0.1% FA):EtOH=80:20; flow: 1.0 mL/min). MS (ESI) m/z: 395 [M+H]⁺. ¹H NMR (400 MHz, DMSO-d(,) δ 15.68 (s, 1H), 8.56 (d, J=4.9 Hz, 1H), 8.04-7.90 (m, 1H), 7.82 (d, J=7.5 Hz, 1H), 7.78-7.61 (m, 1H), 7.59 (d, J=4.9 Hz, 1H), 7.54 (t, J=7.5 Hz, 1H), 7.50-7.21 (m, 2H), 6.43 (d, J=7.2 Hz, 1H), 5.36 (d, J=15.4 Hz, 1H), 4.77 (d, J=9.1 Hz, 1H), 4.60 (d, J=14.0 Hz, 1H), 3.91-3.77 (m, 1H), 2.82 (dd, J=13.9, 9.7 Hz, 1H), 2.19 (s, 3H). The other one of the two enantiomers was a white solid, RT=3.334 min (CHIRAL HPLC, column: CHIRALPAK IH-3; column size: 4.6*50 mm, 3 μm; mobile phase: MtBE(0.1% FA):EtOH=80:20; flow: 1.0 mL/min). MS (ESI) m/z 395 [M+H]⁺. ¹H NMR (400 MHz, DMSO-d₆) δ 15.68 (s, 1H), 8.56 (d, J=4.9 Hz, 1H), 7.95-7.69 (m, 3H), 7.59 (d, J=4.9 Hz, 1H), 7.54 (t, J=7.5 Hz, 1H), 7.45 (t, J=7.5 Hz, 1H), 7.32 (d, J=8.6 Hz, 1H), 6.43 (d, J=7.6 Hz, 1H), 5.36 (d, J=15.4 Hz, 1H), 4.77 (dd, J=9.6, 5.9 Hz, 1H), 4.60 (d, J=15.4 Hz, 1H), 3.85 (dd, J=13.9, 5.9 Hz, 1H), 2.82 (dd, J=13.9, 9.7 Hz, 11H), 2.20 (s, 31H).

### 2-((1H-benzo[d][1,2,3]triazol-5-yl)methyl)-3-((4-chloro-1-methyl-1H-pyrazol-5-yl)methyl)-5-(oxetan-3-yl)isoindolin-1-one (370)

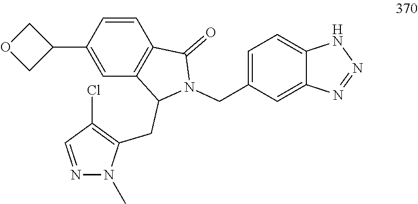 [SMILES: Cn1ncc(Cl)c1CC1c2cc(C3COC3)ccc2C(=O)N1Cc1ccc2[nH]nnc2c1]

The title compound 370 was prepared according to the procedure described for compound 279 as a white solid (30 mg, 38.9%). MS (ESI) m/z 449 [M+H]⁺. ¹H NMR (400 MHz, CDCl₃) δ 7.93 (d, J=7.8 Hz, 11H), 7.87 (d, J=8.4 Hz, 11H), 7.69 (s, 1H), 7.54-7.46 (m, 2H), 7.29 (d, J=8.4 Hz, 1H), 6.75 (s, 1H), 5.54 (d, J=15.0 Hz, 1H), 5.09-5.02 (m, 2H), 4.71 (t, J=7.6 Hz, 11H), 4.67-4.61 (m, 2H), 4.35 (d, J=15.1 Hz, 1H), 4.26-4.16 (m, 1H), 3.48 (s, 3H), 3.39 (dd, J=14.2, 7.2 Hz, 1H), 2.83 (dd, J=14.2, 8.4 Hz, 11H).

The intermediate 370-4 was prepared as follows:

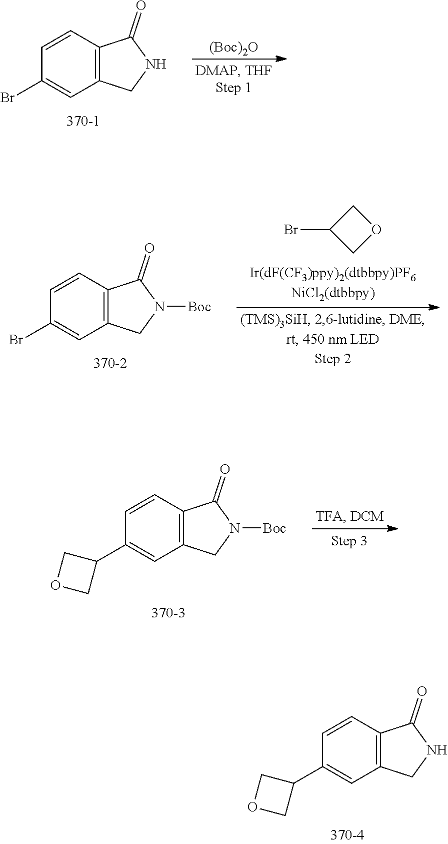 [SMILES: "(Boc)2O" ; O=C1NCc2cc(Br)ccc21 ; CC(C)(C)OC(=O)N1Cc2cc(Br)ccc2C1=O ; BrC1COC1 ; CC(C)(C)OC(=O)N1Cc2cc(C3COC3)ccc2C1=O ; O=C1NCc2cc(C3COC3)ccc21]

Step 1 was performed according to the procedure outlined for preparation of Int 12-1.

Step 2 was performed according to the procedure outlined for preparation of 198-1.

Step 3 was performed according to the procedure outlined for preparation of Int 12.

### 2-((1H-benzo[d][1,2,3]triazol-5-yl)methyl)-3-((4-chloro-3-(2-methoxyethoxy)-1-methyl-1H-pyrazol-5-yl)methyl)isoindolin-1-one (371)

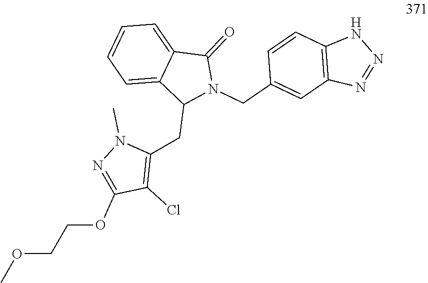 [SMILES: COCCOc1nn(C)c(CC2c3ccccc3C(=O)N2Cc2ccc3[nH]nnc3c2)c1Cl]

The title compound 371 was prepared according to the procedure described for compound 279 as a white solid (9 mg, 19.3%). MS (ESI) m/z 467 [M+H]⁺. ¹H NMR (400 MHz, DMSO-d₆) δ 7.85 (d, J=8.5 Hz, 1H), 7.79-7.71 (m, 2H), 7.59-7.48 (m, 2H), 7.24 (d, J=8.6 Hz, 1H), 7.00-6.93 (m, 1H), 5.35 (d, J=15.3 Hz, 1H), 4.65 (dd, J=8.1, 5.8 Hz, 1H), 4.51 (d, J=15.3 Hz, 1H), 4.26-4.20 (m, 2H), 3.66-3.59 (m, 2H), 3.48 (dd, J=14.9, 5.8 Hz, 1H), 3.40 (s, 3H), 3.29 (s, 3H), 2.97 (dd, J=14.9, 8.1 Hz, 1H).

The intermediate 371-5 was prepared as follows:

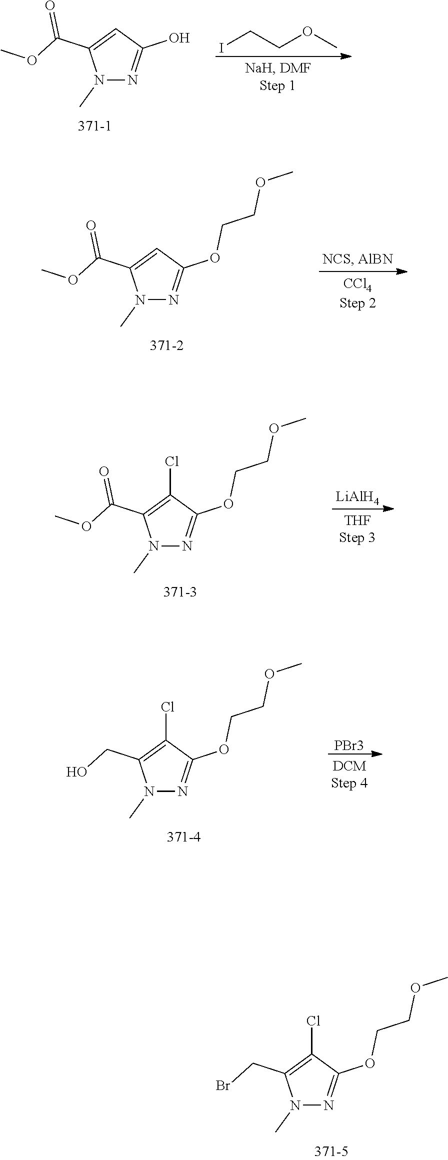

Step 1 was performed according to the procedure outlined for preparation of Int 5.

Step 2 was performed according to the procedure outlined for preparation of 222-1.

Step 3 was performed according to the procedure outlined for preparation of 263-2.

Step 4 was performed according to the procedure outlined for preparation of 263-3.

### 2-((1H-benzo[d][1,2,3]triazol-5-yl)methyl)-3-((4-chloro-1-ethyl-1H-pyrazol-5-yl)methyl)isoindolin-1-one (372) and 2-((1H-benzo[d][1,2,3]triazol-5-yl)methyl)-3-((4-chloro-1-ethyl-1H-pyrazol-3-yl)methyl)isoindolin-1-one (373)

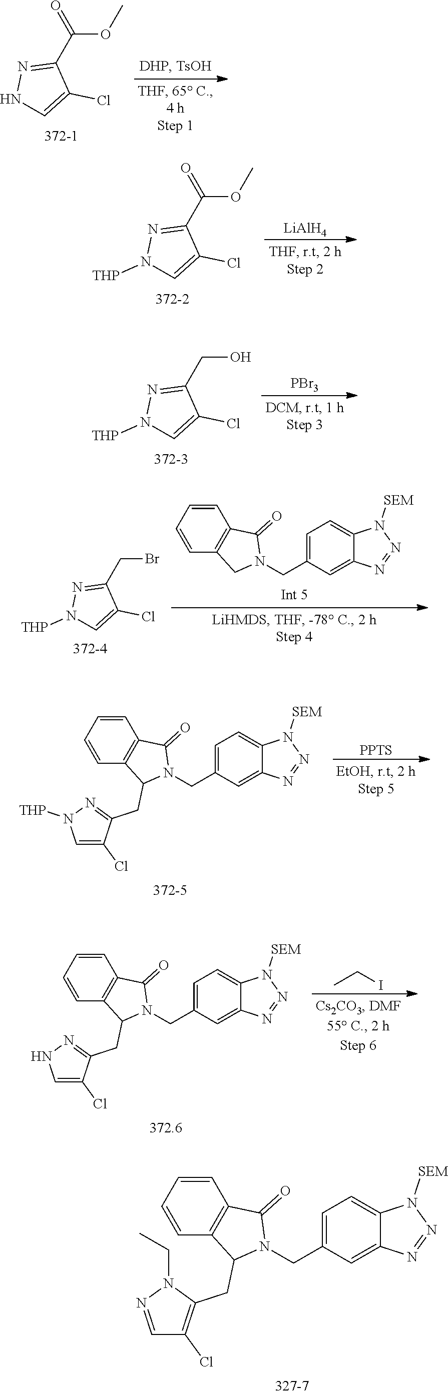

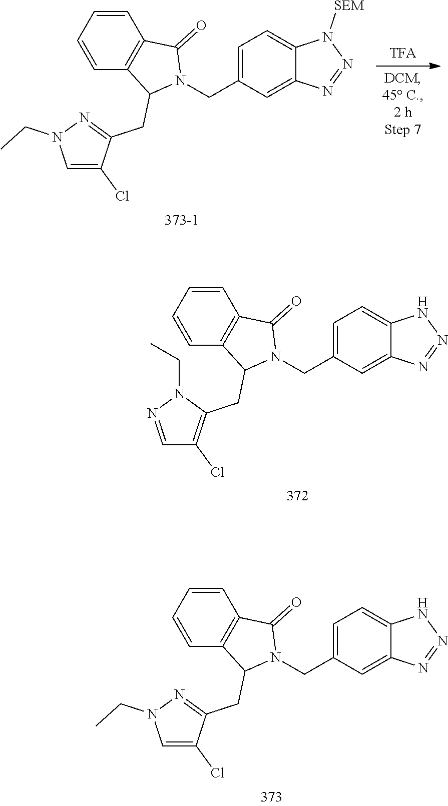 [SMILES: CCn1cc(Cl)c(CC2c3ccccc3C(=O)N2Cc2ccc3c(c2)nnn3COCC[Si](C)(C)C)n1 ; CCn1ncc(Cl)c1CC1c2ccccc2C(=O)N1Cc1ccc2[nH]nnc2c1 ; CCn1cc(Cl)c(CC2c3ccccc3C(=O)N2Cc2ccc3[nH]nnc3c2)n1]

### Step 1. methyl 4-chloro-1-(tetrahydro-2H-pyran-2-yl)-1H-pyrazole-3-carboxylate (372-2)

To a solution of methyl 4-chloro-1H-pyrazole-3-carboxylate (3 g, 18.7 mmol) in THF (50 mL) were added 3,4-Dihydro-2H-pyran (2.35 g, 28.0 mmol) and TsOH (483 mg, 2.8 mmol). The mixture was stirred at 65° C. for 4 h. The reaction mixture was diluted with water and extracted with ethyl acetate. The combined organic phase was dried and concentrated under vacuum to afford 372-2 (4.8 g, crude) as a yellow oil, which was used in the next step without further purification. MS (ESI) m/z 245 [M+H]⁺.

Step 2 was performed according to the procedure outlined for preparation of 263-2.

Step 3 was performed according to the procedure outlined for preparation of 263-3.

Step 4 was performed according to the procedure outlined for preparation of Int 12-2.

### Step 5. 3-((4-chloro-1H-pyrazol-3-yl)methyl)-2-((1-((2-(trimethylsilyl)ethoxy) methyl)-1H-benzo[d][1,2,3]triazol-5-yl)methyl)isoindolin-1-one (372-6)

To a solution of 372-5 (200 mg, 0.34 mmol) in EtOH (5 mL) was added PPTS (58 mg, 0.34 mmol). The mixture was stirred at r.t. for 2h, and then concentrated under vacuum. The residue was purified by silica gel column eluting with PE/EA=1/2 to afford 372-6 (76 mg, 44%) as a light yellow oil. MS (ESI) m/z 509 [M+H]⁺.

### Step 6. Mixture of 3-((4-chloro-1-ethyl-1H-pyrazol-5-yl)methyl)-2-((1-((2-(trimethylsilyl)ethoxy)methyl)-1H-benzo[d][1,2,3]triazol-5-yl)methyl)isoindolin-1-one (372-7) and 3-((4-chloro-1-ethyl-1H-pyrazol-3-yl)methyl)-2-((1-((2-(trimethylsilyl)ethoxy)methyl)-1H-benzo[d][1,2,3]triazol-5-yl)methyl)isoindolin-1-one (373-1)

To a solution of 372-6 (76 mg, 0.15 mmol) in DMF (2 mL) were added Cs₂CO₃ (97 mg, 0.30 mmol) and iodoethane (35 mg, 0.22 mmol). The resulting mixture was stirred at 55′C for 2 h. The reaction mixture was diluted with water and extracted with ethyl acetate. The combined organic phase was washed with water and brine, dried over anhydrous Na₂SO₄ and concentrated under vacuum to afford a mixture of 372-7 and 373-1 (120 mg, crude) as a light yellow oil. MS (ESI) m/z 537 [M+H]⁺.

### Step 7. 2-((1H-benzo[d][1,2,3]triazol-5-yl)methyl)-3-((4-chloro-1-ethyl-1H-pyrazol-5-yl)methyl)isoindolin-1-one (372) and 2-((1H-benzo[d][1,2,3]triazol-5-yl)methyl)-3-((4-chloro-1-ethyl-1H-pyrazol-3-yl)methyl)isoindolin-1-one (373)

To a solution of a mixture of 372-7 and 373-1 (120 mg, crude) in DCM (3 mL) was added 20 trifluoroacetic acid (15 mL). The mixture was stirred at 45° C. for 2h, and then concentrated under vacuum. The residue was purified by Prep-HPLC to afford two compounds: compound 372 and compound 373. One of the two compounds was a white solid (3 mg, 3.3%), with RT=4.48 min (HPLC). MS (ESI) m/z 407 [M+H]⁺. ¹H NMR (400 MHz, CD₃OD) δ 7.97-7.81 (m, 2H), 7.76 (s, 1H), 7.62-7.43 (m, 3H), 7.36 (d, J=8.6 Hz, 1H), 6.79 (d, J=7.3 Hz, 1H), 5.55 (d, J=15.3 Hz, 1H), 4.74 (dd, J=8.7, 6.2 Hz, 1H), 4.54 (d, J=14.5 Hz, 1H), 3.87-3.68 (m, 2H), 3.55 (dd, J=14.9, 6.2 Hz, 1H), 2.97 (dd, J=14.8, 8.7 Hz, 1H), 1.13 (t, J=7.2 Hz, 3H). The other one of the two compounds was a white solid (5 mg, 5.6%), with RT=4.56 min (HPLC). MS (ESI) m/z 407 [M+H]⁺. ¹H NMR (400 MHz, CD₃OD) δ 7.83 (d, J=8.6 Hz, 1H), 7.80-7.75 (m, 1H), 7.69 (s, 1H), 7.58 (s, 1H), 7.56-7.45 (m, 2H), 7.35 (dd, J=8.6, 1.5 Hz, 1H), 7.26 (d, J=7.6 Hz, 1H), 5.46 (d, J=15.6 Hz, 1H), 4.67 (d, J=15.6 Hz, 1H), 4.08-3.98 (m, 2H), 3.32-3.29 (m, 1H), 3.08 (dd, J=14.8, 6.5 Hz, 1H), 1.34 (t, J=7.3 Hz, 3H).

### 4-((2-((1H-benzo[d][1,2,3]triazol-5-yl)methyl)-3-oxoisoindolin-1-yl)(hydroxy)methyl)-5-methylisoxazole-3-carbonitrile (374)

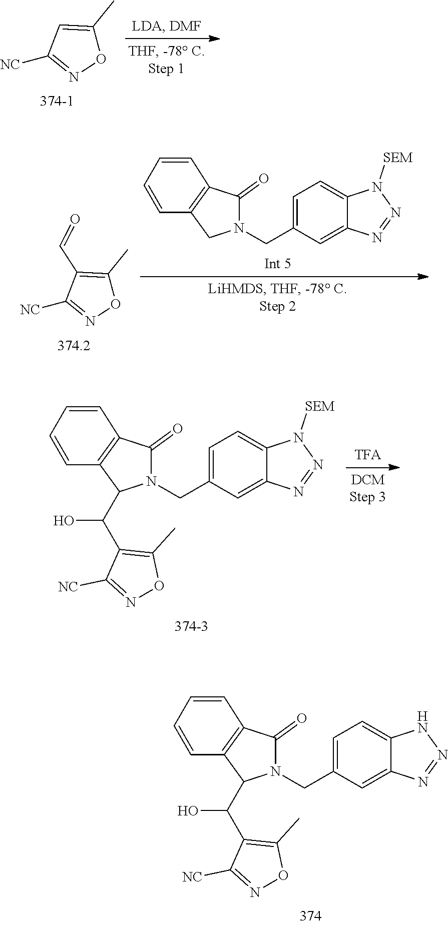

### Step 1: 4-formyl-5-methylisoxazole-3-carbonitrile (374-2)

To a solution of 5-methylisoxazole-3-carbonitrile (1 g, 9.3 mmol) in THF (15 mL) was added LDA (5.1 mL, 2M/L) at −78° C. under N₂. The mixture was stirred for 30 min at −78° C., then to the mixture was added DMF (3.7 g, 45 mmol) and the mixture was stirred for 2 h at −78° C. The mixture was quenched with NH₄Cl (aq.) (20 mL) and extracted with Ethyl acetate (2*25 mL). The combined organic layer was dried over anhydrous Na₂SO₄ and concentrated. The residue was purified by column chromatography on silica gel (eluting (PE/EA=10/1 to 5/1) to give 374-2 (330 mg, 26%) as a white solid. MS (ESI) m/z 137 [M+H]⁺.

### Step 2: 4-(hydroxy(3-oxo-2-((1-((2-(trimethylsilyl)ethoxy)methyl)-1H-benzo[d][1,2,3]triazol-5-yl)methyl)isoindolin-1-yl)methyl)-5-methylisoxazole-3-carbonitrile (374-3)

To a solution of Int 5 (300 mg, 0.76 mmol) in dried THF (5 mL) was added dropwise LiHMDS (1.5 mL, 1M/L in THF) at −78° C. under N₂. The mixture was stirred for 40 min at −78° C., then added 374-2 (122 mg, 0.9 mmol). The mixture was stirred for 1 h at −78° C. The mixture was quenched with NH₄Cl (aq.) (5 mL) and extracted with Ethyl acetate (2*5 mL). The combined organic layer was dried over anhydrous Na₂SO₄ and concentrated. The residue was purified by column chromatography on silica gel (eluting (PE/EA=4/1 to 1/1) to give 374-3 (170 mg, 42%) as a yellow oil. MS (ESI) m/z 531 [M+H]⁺.

Step 3 was performed according to the procedure outlined for preparation of compound 7 to afford 374 as a white solid (45 mg, 34%). MS (ESI) m/z 401 [M+H]⁺. ¹H NMR (400 MHz, DMSO-d₆) δ 7.87 (d, J=8.5 Hz, 1H), 7.73 (s, 1H), 7.65 (d, J=7.2 Hz, 1H), 7.55 (dd, J=7.4, 1.4 Hz, 1H), 7.51-7.43 (m, 2H), 7.30 (dd, J=8.6, 1.5 Hz, 1H), 6.56 (bs, 1H), 5.57 (d, J=3.2 Hz, 1H), 5.50 (d, J=15.1 Hz, 1H), 4.94 (d, J=15.1 Hz, 1H), 4.79 (d, J=3.2 Hz, 1H), 2.13 (s, 3H).

### 2-((1H-benzo[d][1,2,3]triazol-5-yl)methyl)-3-((2-(2-hydroxypropan-2-yl)-4-methylpyridin-3-yl)methyl)isoindolin-1-one (375)

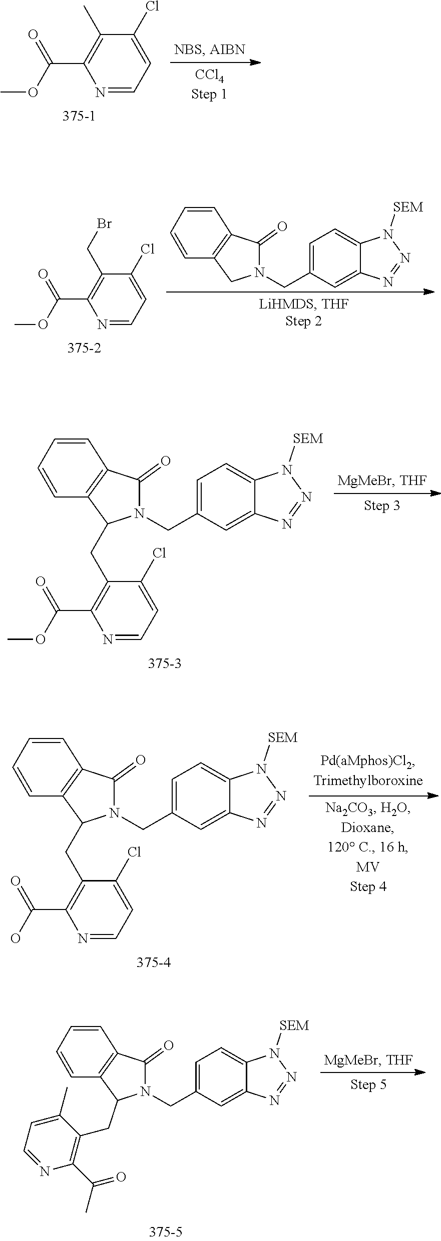

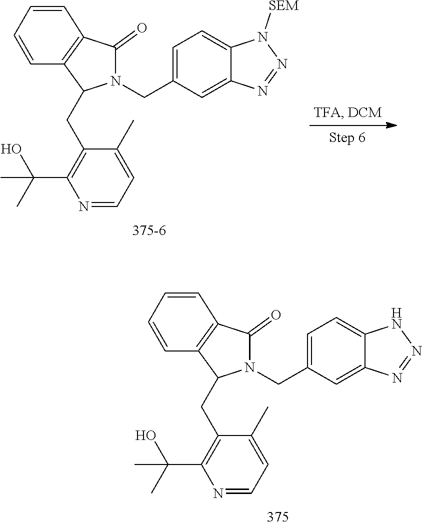 [SMILES: Cc1ccnc(C(C)(C)O)c1CC1c2ccccc2C(=O)N1Cc1ccc2c(c1)nnn2COCC[Si](C)(C)C ; Cc1ccnc(C(C)(C)O)c1CC1c2ccccc2C(=O)N1Cc1ccc2[nH]nnc2c1]

Step 1 was performed according to the procedure outlined for preparation of 222-1.

Step 2 was performed according to the procedure outlined for preparation of Int 12-2.

### Step 3. 3-((2-acetyl-4-chloropyridin-3-yl)methyl)-2-((1-((2-(trimethylsilyl) ethoxy)methyl)-1H-benzo[d][1,2,3]triazol-5-yl)methyl)isoindolin-1-one (375-4)

To a solution of 375-3 (310 mg, 0.5 mmol) in THF was added dropwise methylmagnesium bromide (0.2 mL, 3M/L in THF) at 0° C. The mixture was stirred at r.t. for 2 h. Then quenched with NH₄Cl (aq.) (5 mL) and extracted with Ethyl acetate (2*10 mL). The combined organic layer was dried over anhydrous Na₂SO₄ and concentrated. The residue was purified by column chromatography on silica gel (eluting with PE/EA=1/1 to 1/2) to give 375-4 (250 mg, 83%) as a yellow oil. MS (ESI) m/z 562 [M+H]⁺.

### Step 4. 3-((2-acetyl-4-methylpyridin-3-yl)methyl)-2-((1-((2-(trimethylsilyl)ethoxy) methyl)-1H-benzo[d][1,2,3]triazol-5-yl)methyl)isoindolin-1-one (375-5)

A mixture of 375-4 (170 mg, 0.3 mmol), Pd(amphos)C₁₂ (43 mg, 0.06 mmol), Trimethylboroxine (180 mg, 1.43 mmol), and Na₂CO₃ (80 mg, 0.75 mmol) in THF/H₂O (4/1 mL) was reacted at 120° C. for 2 h under N₂ using microwave reactor. The mixture was filtered, the filtrate was extracted with EA (2*20 mL). The combined organic layer was dried over anhydrous Na₂SO₄ and concentrated. The residue was purified by column chromatography on silica gel (eluting with PE/EA=1/1 to 1/2) to give 375-5 (110 mg, 67%) as a yellow oil. MS (ESI) m/z 542 [M+H]⁺.

Step 5 was performed according to the procedure outlined for preparation of 375-4.

Step 6 was performed according to the procedure outlined for step 2 for preparation of compound 7 to afford 375 as a white solid (22 mg, 32%). MS (ESI) m/z 428 [M+H]⁺. ¹H NMR (400 MHz, CDCl₃) δ 8.37 (d, J=4.3 Hz, 1H), 7.92 (d, J=7.6 Hz, 1H), 7.83 (d, J=8.3 Hz, 1H), 7.64 (s, 1H), 7.43 (t, J=7.5 Hz, 1H), 7.33-7.27 (m, 2H), 7.07 (s, 1H), 6.33 (bs, 1H), 5.55 (d, J=15.2 Hz, 1H), 4.91-4.81 (m, 1H), 4.38-4.23 (m, 1H), 3.76-3.62 (m, 1H), 3.23-3.07 (m, 1H), 1.42 (s, 3H), 1.25 (s, 6H).

### (2r,4r)-2-((1-((3-bromopyridin-2-yl)methyl)-3-oxoisoindolin-2-yl)methyl)-2-methyl-5-oxa-7-azaspiro[3.4]octan-6-one (376) and (2s,4s)-2-((1-((3-bromopyridin-2-yl)methyl)-3-oxoisoindolin-2-yl)methyl)-2-methyl-5-oxa-7-azaspiro[3.4]octan-6-one (377)

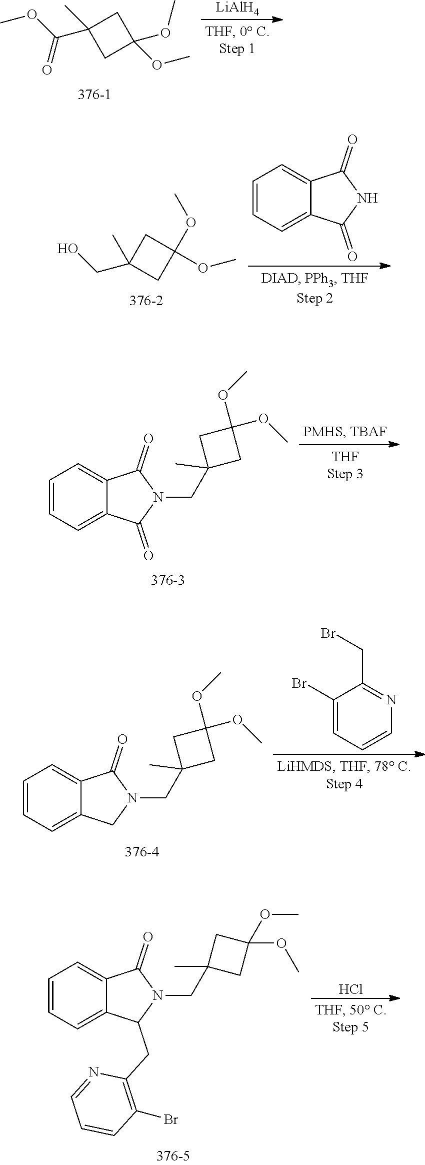

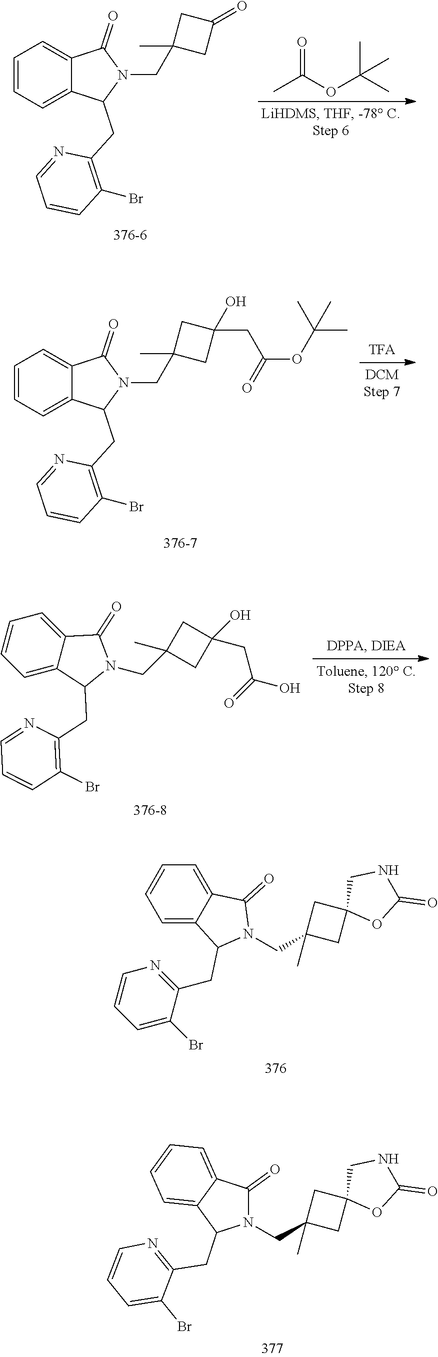

Step 1 was performed according to the procedure outlined for preparation of 263-2.

### Step 2. 2-((3,3-dimethoxy-1-methylcyclobutyl)methyl)isoindoline-1,3-dione (376-3)

To a solution of isoindoline-1,3-dione (1.0 g, 6.80 mmol) in THF (20 mL) were added (3,3-dimethoxy-1-methyl-cyclobutyl)methanol (1.31 g, 8.16 mmol), DIAD (1.65 g, 8.16 mmol) and PPh₃ (2.14 g, 8.16 mmol) at 0° C. The mixture was stirred at r.t. for 2 h. The reaction was monitored by LC/MS. Once completed, the solvent was removed under reduced pressure and the residue was purified by column chromatograph (PE/EA=1/1) to yield 376-3 (1.27 g, 64%). ¹H NMR (400 MHz, CDCl₃) δ 7.93-7.80 (m, 2H), 7.80-7.65 (m, 2H), 3.75 (s, 2H), 3.09 (dd, J=14.9, 1.4 Hz, 6H), 2.42-2.27 (m, 2H), 1.99-1.82 (m, 2H), 1.21 (d, J=1.4 Hz, 3H).

### Step 3. 2-((3,3-dimethoxy-1-methylcyclobutyl)methyl)isoindolin-1-one (376-4)

To a solution of 376-3 (1.24 g, 4.29 mmol) in THF (15 mL) were added TBAF (68 mg, 0.21 mmol) and PMHS (1.3 mL, 21.4 mmol) under Ar. The mixture was stirred at 25° C. overnight. The reaction was monitored by LC/MS. Once completed, the precipitate was filtered, and the filtrate was concentrated under reduced pressure. The residue was purified by column chromatograph (PE/EA=1/1) to yield 376-4 (446 mg, 38%). MS (ESI) m/z 276 [M+H]⁺.

Step 4 was performed according to the procedure outlined for preparation of Int 12-2.

Step 5 was performed according to the procedure outlined for preparation of 270-3.

### Step 6. tert-Butyl 2-(3-((1-((3-bromopyridin-2-yl)methyl)-3-oxoisoindolin-2-yl)methyl)-1-hydroxy-3-methylcyclobutyl)acetate (376-7)

To a solution of tert-butyl acetate (0.041 mL, 0.31 mmol) in THF (3 mL) was added LiHMDS (54 mg, 0.32 mmol) at −78° C. and the mixture was stirred for 1 h. Then 376-6 (61 mg, 0.15 mmol) was added and the mixture was stirred for 2 h at −78° C. The reaction was monitored by LC/MS. Once completed, the solvent was removed under reduced pressure and the residue was dissolved in DCM, washed with water, dried over Na₂SO₄, filtered and concentrated under reduced pressure. The residue was used in the next step without further purification.

Step 7 was performed according to the procedure outlined for preparation of 270-5.

Step 8 was performed according to the procedure outlined for step 5 for preparation of 270. Two products were obtained after purified by prep-HPLC. One of the two products was a white solid (2 mg, 2.5%), with RT=4.40 min (HPLC). MS (ESI) m/z 456 [M+H]⁺. ¹H NMR (400 MHz, DMSO-d₆) δ 8.60 (dd, J=4.6, 1.5 Hz, 1H), 8.10 (dd, J=8.1, 1.5 Hz, 1H), 7.75-7.58 (m, 1H), 7.55-7.38 (m, 2H), 7.31 (dd, J=8.1, 4.6 Hz, 1H), 6.91-6.78 (m, 1H), 5.26 (dd, J=8.4, 4.7 Hz, 1H), 4.03 (d, J=14.3 Hz, 1H), 3.78 (dd, J=14.8, 4.8 Hz, 1H), 3.57 (s, 1H), 3.08 (dd, J=14.8, 8.4 Hz, 1H), 2.72-2.62 (m, 1H), 2.44 (m, 2H), 2.37-2.27 (m, 1H), 2.12 (t, J=11.5 Hz, 2H), 0.98 (s, 3H). The other one of the two products was a white solid (3 mg, 3.9%), with RT=4.54 min (HPLC). MS (EST) m/z 456 [M+H]⁺. ¹H NMR (400 MHz, DMSO-d₆) δ 8.59 (dd, J=4.6, 1.5 Hz, 1H), 8.08 (dd, J=8.1, 1.5 Hz, 1H), 7.73-7.61 (m, 1H), 7.49-7.39 (m, 2H), 7.30 (dd, J=8.1, 4.6 Hz, 1H), 6.91-6.81 (m, 1H), 5.28 (dd, J=8.2, 4.7 Hz, 1H), 4.01 (d, J=14.5 Hz, 1H), 3.78 (dd, J=14.8, 4.8 Hz, 1H), 3.58 (d, J=9.3 Hz, 1H), 3.48 (d, J=9.3 Hz, 1H), 3.24 (m, 1H), 3.08 (dd, J=14.7, 8.3 Hz, 1H), 2.45-2.29 (m, 2H), 2.22-2.03 (m, 2H), 1.13 (s, 3H).

### (R)-2-((1H-benzo[d][1,2,3]triazol-5-yl)methyl)-3-((4-chloro-1-(oxetan-3-yl)-1H-pyrazol-3-yl)methyl)isoindolin-1-one (378) and (S)-2-((1H-benzo[d][1,2,3]triazol-5-yl)methyl)-3-((4-chloro-1-(oxetan-3-yl)-1H-pyrazol-3-yl)methyl)isoindolin-1-one (379)

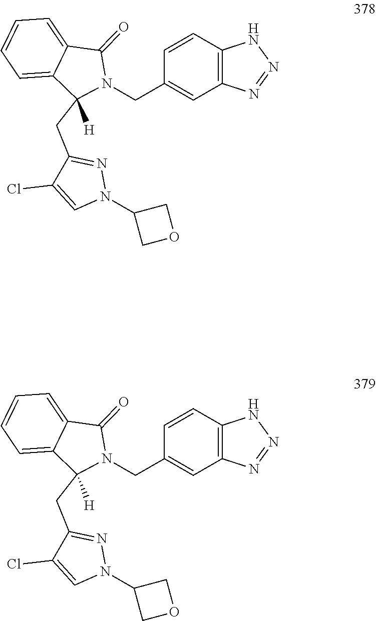 [SMILES: O=C1c2ccccc2[C@@H](Cc2nn(C3COC3)cc2Cl)N1Cc1ccc2[nH]nnc2c1 ; O=C1c2ccccc2[C@H](Cc2nn(C3COC3)cc2Cl)N1Cc1ccc2[nH]nnc2c1]

Chiral separation of compound 318 by chiral prep-HPLC (column: CHIRALPAK IH; column size: 2 cm*25 cm, Sum; mobile phase A: MTBE; mobile phase B: EtOH) was conducted to afford two enantiomers: compound 378 and compound 379. One of the two enantiomers was a white solid, with RT=3.572 min (CHIRAL HPLC, column: CHIRALPAK IH-3; column size: 4.6*50 mm, 3 μm; mobile phase: MTBE (0.1% DEA):(EtOH)=80:20; flow: 1 mL/min). MS (ESI) m/z 435 [M+H]⁺. The other one of the two enantiomers was a white solid, with RT=5.826 min (CHIRAL HPLC, column: CHIRALPAK IH-3; column size: 4.6*50 mm, 3 μm; mobile phase: MTBE (0.1% DEA):(EtOH)=80:20; flow: 1 mL/min). MS (ESI) m/z 435 [M+H]⁺.

### (E)-2-(3-(1H-1,2,3-triazol-4-yl)allyl)-3-((2-bromo-4-methylpyridin-3-yl)methyl) isoindolin-1-one (380)

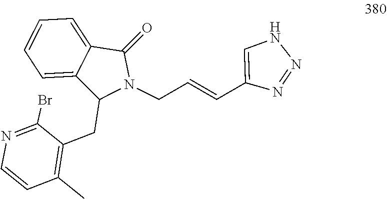 [SMILES: Cc1ccnc(Br)c1CC1c2ccccc2C(=O)N1C/C=C/c1c[nH]nn1]

The title compound 380 was prepared according to the procedure described for compound 312-5 as a brown solid (280 mg, 47%). MS (ESI) m/z 424 [M+H]⁺. ¹H NMR (400 MHz, DMSO-d₆) δ 8.23 (d, J=4.8 Hz, 1H), 7.93 (d, J=1.7 Hz, 1H), 7.76 (d, J=7.3 Hz, 1H), 7.56-7.41 (m, 2H), 7.32 (d, J=4.9 Hz, 1H), 6.60 (d, J=7.5 Hz, 1H), 6.55 (d, J=16.0 Hz, 1H), 6.43-6.32 (m, 1H), 5.04 (dd, J=9.5, 5.9 Hz, 1H), 4.79-4.68 (m, 1H), 3.94 (dd, J=16.0, 7.7 Hz, 1H), 3.62 (dd, J=13.8, 6.5 Hz, 1H), 2.88 (dd, J=12.0, 8.0 Hz, 1H), 2.12 (s, 3H).

### 2-((1H-benzo[d][1,2,3]triazol-5-yl)methyl)-3-((3-chloro-1-methyl-6-oxo-1,6-dihydropyridin-2-yl)methyl)isoindolin-1-one (381)

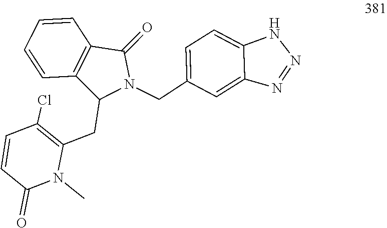 [SMILES: Cn1c(CC2c3ccccc3C(=O)N2Cc2ccc3[nH]nnc3c2)c(Cl)ccc1=O]

The title compound 381 was prepared according to the procedure described for compound 279 as a white solid (13 mg, 31%). MS (ESI) m/z 420 [M+H]⁺. ¹H NMR (400 MHz, CDCl₃) δ 7.97 (d, J=7.4 Hz, 1H), 7.87 (d, J=8.5 Hz, 1H), 7.72 (s, 1H), 7.60-7.44 (m, 2H), 7.36 (d, J=9.6 Hz, 1H), 7.32 (dd, J=8.4, 1.6 Hz, 1H), 6.83 (d, J=7.5 Hz, 1H), 6.65 (d, J=9.7 Hz, 1H), 5.56 (d, J=15.3 Hz, 1H), 4.83 (t, J=7.8 Hz, 1H), 4.39 (d, J=15.4 Hz, 1H), 3.63 (dd, J=13.4, 6.2 Hz, 1H), 3.16 (s, 3H), 2.95 (dd, J=14.3, 8.6 Hz, 1H).

The intermediate 381-8 was prepared as follows:

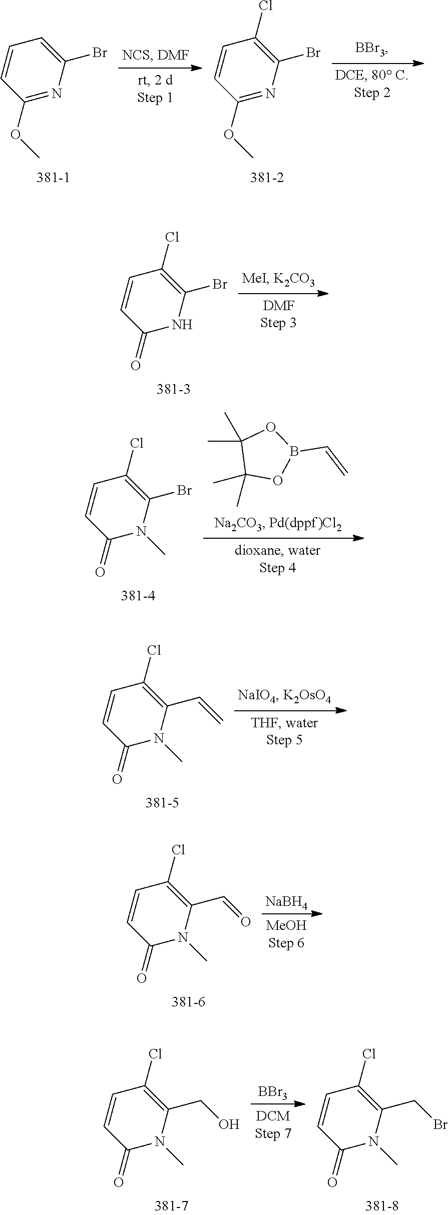

### Step 1: 2-bromo-3-chloro-6-methoxypyridine (381-2)

A mixture of 2-bromo-6-methoxypyridine (5.0 g, 0.02 mol) and NCS (5.2 g, 0.04 mol) in dried DMF (30 mL) was stirred at r.t. for 2 days. The mixture was extracted with ethyl acetate (20 mL*3), the combined organic layer was washed with water (20 mL) and brine (20 mL), dried over Na₂SO₄, filtered, and concentrated in vacuo. The residue was purified by column chromatography on silica gel (eluting with PE/EA=5:1 to 1/1) to give 381-2 (3.7 g, 45%) as a white solid. MS (ESI) m/z 222 [M+H]⁺.

### Step 2: 6-bromo-5-chloropyridin-2(1H)-one (381-3)

To a solution of 381-2 (3.5 g, 1.5 mol) in DCE (30 mL) was added BBr₃ (1.1 g, 4.5 mol). The reaction mixture was stirred at 80° C.T for 2 h. The mixture was diluted with water (30 mL) and extracted with ethyl acetate (20 mL*2). The combined organic layer was washed with water (10 mL) and brine (10 mL), dried over Na₂SO₄, filtered, and concentrated in vacuo. The residue was purified by column chromatography on silica gel (eluting with PE/EA=3/1 to 1/1) to give 381-3 (1.5 g, 65%) as a yellow oil. MS (ESI) m/z 208 [M+H]⁺.

### Step 3. 6-bromo-5-chloro-1-methylpyridin-2(1H)-one (381-4)

A mixture of 381-3 (1.5 g, 7.2 mmol), Mel (2.0 g, 14.4 mmol) and K₂CO₃ (1.4 g, 14.4 mmol) in DMF (30 mL) was stirred for 16 h at 25° C. The mixture was diluted with water (30 mL) and extracted with ethyl acetate (10 mL*2). The combined organic layer was washed with water (2*10 mL) and brine (2*10 mL), dried over Na₂SO₄, filtered, and concentrated in vacuo. The residue was purified by column chromatography on silica gel (eluting with PE/EA=5/1 to 1/1) to give 381-4 (600 mg, 35%) as a white oil. MS (ESI) m/z 222 [M+H]⁺.

Step 4 was performed according to the procedure outlined for the preparation of 26-1.

### Step 5. 3-chloro-1-methyl-6-oxo-1,6-dihydropyridine-2-carbaldehyde (381-6)

To a solution of 5-chloro-1-methyl-6-vinylpyridin-2(1H)-one (290 mg, 1.7 mmol) in THF/H₂O (18 mL) were added NaIO₄ (1.1 g, 5.4 mmol) and K₂OsO₄ (35 mg, 0.1 mmol). The mixture was stirred at 25′C for 16 h. The mixture was diluted with water (10 mL) and extracted with ethyl acetate (2*10 mL). The combined organic layer was dried over anhydrous Na₂SO₄ and concentrated. The residue was purified by column chromatography on silica gel (eluting with PE/EA=4/1 to 1/1) to give 381-6 (280 mg, 89%) as a yellow solid. MS (ESI) m/z 172 [M+H]⁺.

Step 6 was performed according to the procedure outlined for preparation of 164-2.

Step 7 was performed according to the procedure outlined for preparation of 263-3.

### 2-((1H-benzo[d][1,2,3]triazol-5-yl)methyl)-3-((4-chloro-1-methyl-1H-pyrazol-5-yl)methyl)-5-(2-hydroxypropan-2-yl)isoindolin-1-one (382)

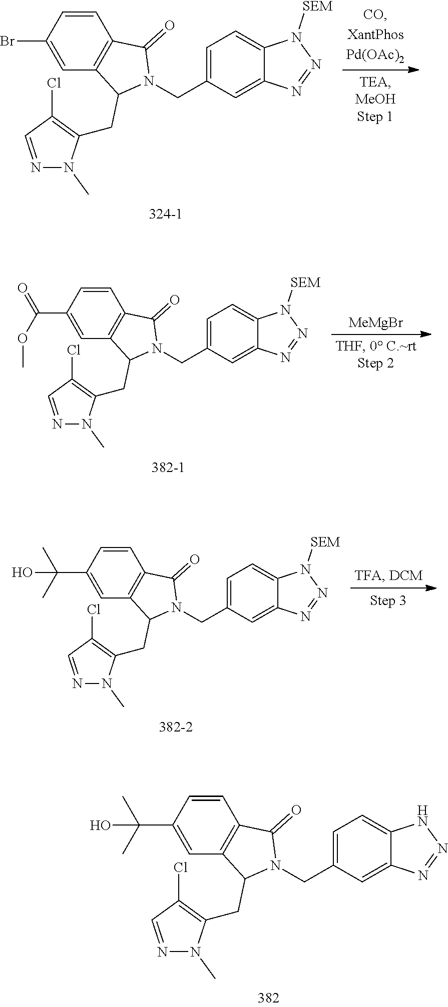 [SMILES: "MeMgBr" ; Cn1ncc(Cl)c1CC1c2cc(Br)ccc2C(=O)N1Cc1ccc2c(c1)nnn2COCC[Si](C)(C)C ; COC(=O)c1ccc2c(c1)C(Cc1c(Cl)cnn1C)N(Cc1ccc3c(c1)nnn3COCC[Si](C)(C)C)C2=O ; Cn1ncc(Cl)c1CC1c2cc(C(C)(C)O)ccc2C(=O)N1Cc1ccc2c(c1)nnn2COCC[Si](C)(C)C ; Cn1ncc(Cl)c1CC1c2cc(C(C)(C)O)ccc2C(=O)N1Cc1ccc2[nH]nnc2c1]

### Step 1. methyl 3-((4-chloro-1-methyl-1H-pyrazol-5-yl)methyl)-1-oxo-2-((1-((2-10 (trimethylsilyl)ethoxy)methyl)-1H-benzo[d][1,2,3]triazol-5-yl)methyl)isoindoline-5-carboxylate (382-1)

A mixture of 324-1 (300 mg, 0.5 mmol), Xantphos (58 mg, 0.1 mmol), Pd(OAc)₂ (8 mg, 0.05 mmol), and TEA (2 ml) in MeOH (6 mL) was stirred at 85 Tc for 16 h under CO (balloon) atmosphere. After cooled, the mixture was filtered, and the filtrate was concentrated. The residue was purified by column chromatography on silica gel (eluting methanol/dichloromethane=1/100) to give 382-1 (174 mg, 60% o) as a yellow oil. MS (ESI) m/z 581 [M+H]⁺.

Step 2 was performed according to the procedure outlined for preparation of 375-4.

Step 3 was performed according to the procedure outlined for step 2 for the preparation of compound 7 to afford 382 as a white solid (8 mg, 13%). MS (ESI) m/z 451 [M+H]⁺. ¹H NMR (400 MHz, CDCl₃) δ 7.89 (d, J=8.1 Hz, 1H), 7.87-7.83 (m, 1H), 7.68 (s, 1H), 7.63 (dd, J=8.0, 1.6 Hz, 1H), 7.52-7.49 (m, 1H), 7.35 (d, J=9.1 Hz, 1H), 6.76 (s, 1H), 5.54 (d, J=15.2 Hz, 1H), 4.65 (dd, J=8.6, 6.6 Hz, 1H), 4.35 (d, J=15.2 Hz, 1H), 3.50-3.36 (in, 1H), 3.43 (s, 3H), 2.75 (dd, J=14.6, 8.7 Hz, 1H), 1.52 (s, 3H), 1.51 (s, 3H).

### 2-((1H-benzo[d][1,2,3]triazol-5-yl)methyl)-5-(azetidin-3-yl)-3-((4-chloro-1-methyl-1H-pyrazol-5-yl)methyl)isoindolin-1-one (383)

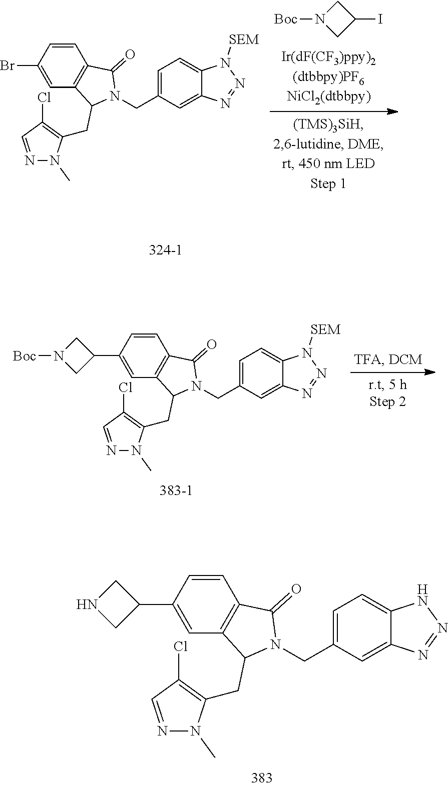 [SMILES: CC(C)(C)OC(=O)N1CC(I)C1 ; Cn1ncc(Cl)c1CC1c2cc(Br)ccc2C(=O)N1Cc1ccc2c(c1)nnn2COCC[Si](C)(C)C ; Cn1ncc(Cl)c1CC1c2cc(C3CN(C(=O)OC(C)(C)C)C3)ccc2C(=O)N1Cc1ccc2c(c1)nnn2COCC[Si](C)(C)C ; Cn1ncc(Cl)c1CC1c2cc(C3CNC3)ccc2C(=O)N1Cc1ccc2[nH]nnc2c1]

Step 1 was performed according to the procedure outlined for preparation of 198-1.

Step 2 was performed according to the procedure outlined for step 2 for the preparation of compound 7 to afford 383 as a white solid (23 mg, 35%). MS (ESI) m/z 448 [M+H]⁺. ¹H NMR (400 MHz, CD₃OD) δ 7.85 (d, J=8.3 Hz, 1H), 7.73 (d, J=7.7 Hz, 1H), 7.68 (s, 1H), 7.54 (s, 1H), 7.49 (d, J=7.7 Hz, 1H), 7.21 (d, J=8.8 Hz, 1H), 6.90 (s, 1H), 5.36 (d, J=15.2 Hz, 11H), 4.63 (t, J=6.9 Hz, 1H), 4.44 (d, J=15.1 Hz, 1H), 3.99 (s, 3H), 3.85-3.70 (m, 1H), 3.33-3.54 (m, 5H), 3.05 (dd, J=14.6, 7.5 Hz, 1H).

### 5-((2-((1H-benzo[d][1,2,3]triazol-5-yl)methyl)-3-oxoisoindolin-1-yl)methyl)-6-methylpyrimidine-4-carbonitrile (384)

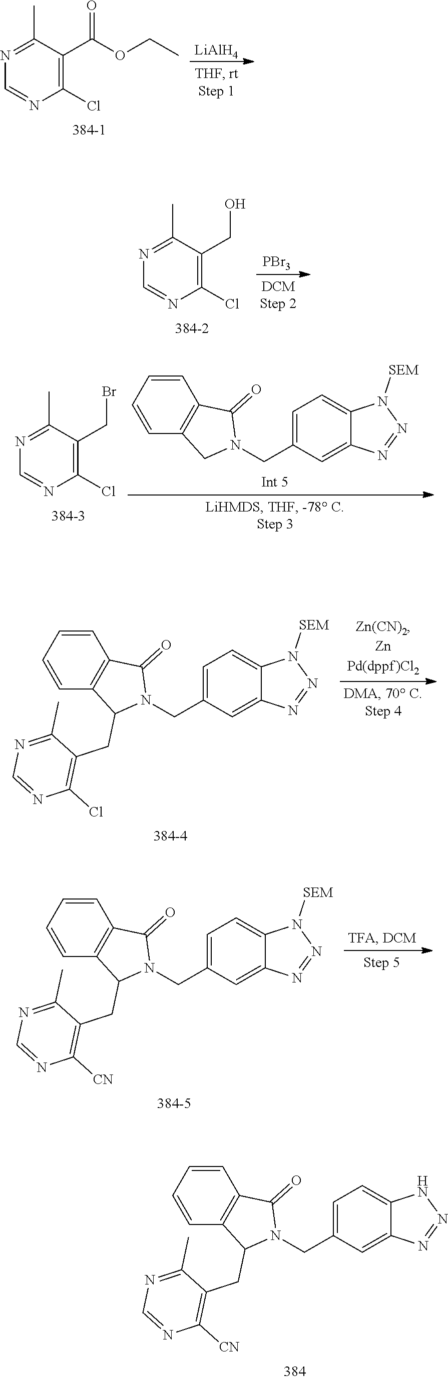

Step 1 was performed according to the procedure outlined for preparation of 263-2.

Step 2 was performed according to the procedure outlined for preparation of 263-3.

Step 3 was performed according to the procedure outlined for preparation of Int 12-2.

Step 4 was performed according to the procedure outlined for preparation of 284-2.

Step 5 was performed according to the procedure outlined for step 2 for preparation of compound 7 to afford 384 as a white solid (15 mg, 40%). MS (ESI) m/z 396 [M+H]⁺. ¹H NMR (400 MHz, CD₃OD) δ 9.07 (s, 1H), 7.94-7.82 (m, 3H), 7.57 (t, J=7.5 Hz, 1H), 7.52-7.40 (m, 2H), 6.60 (d, J=7.6 Hz, 1H), 5.50 (d, J=15.6 Hz, 1H), 4.91-4.87 (m, 1H), 4.72 (d, J=15.6 Hz, 1H), 3.84 (dd, J=14.2, 5.7 Hz, 1H), 2.92 (dd, J=14.1, 9.8 Hz, 1H), 2.32 (s, 3H).

### 2-((1H-benzo[d][1,2,3]triazol-5-yl)methyl)-3-((4-chloro-1-methyl-1H-pyrazol-5-yl)methyl)-5-(1-methylazetidin-3-yl)isoindolin-1-one (385)

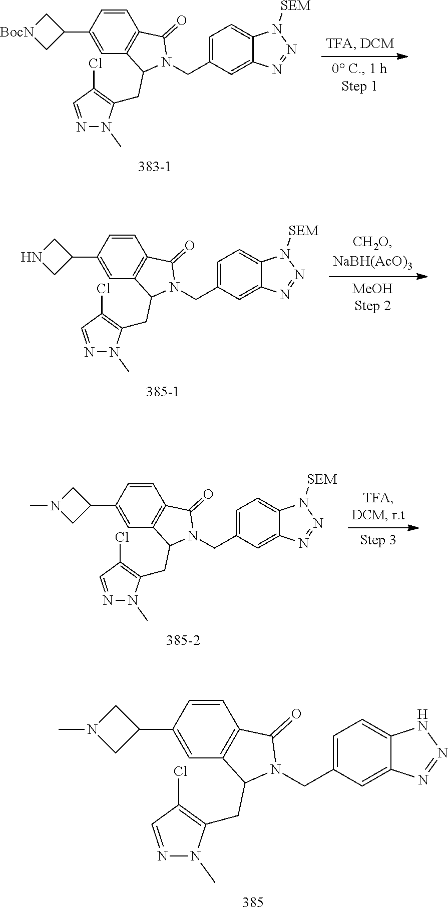 [SMILES: Cn1ncc(Cl)c1CC1c2cc(C3CN(C(=O)OC(C)(C)C)C3)ccc2C(=O)N1Cc1ccc2c(c1)nnn2COCC[Si](C)(C)C ; Cn1ncc(Cl)c1CC1c2cc(C3CNC3)ccc2C(=O)N1Cc1ccc2c(c1)nnn2COCC[Si](C)(C)C ; CN1CC(c2ccc3c(c2)C(Cc2c(Cl)cnn2C)N(Cc2ccc4c(c2)nnn4COCC[Si](C)(C)C)C3=O)C1 ; CN1CC(c2ccc3c(c2)C(Cc2c(Cl)cnn2C)N(Cc2ccc4[nH]nnc4c2)C3=O)C1]

Step 1 was performed according to the procedure outlined for step 2 for preparation of compound 7.

### Step 2: 3-((4-chloro-1-methyl-1H-pyrazol-5-yl)methyl)-5-(1-methylazetidin-3-yl)-2-((1-((2-(trimethylsilyl)ethoxy)methyl)-1H-benzo[d][1,2,3]triazol-5-yl)methyl)isoindolin-1-one (385-2)

A mixture of 385-1 (50 mg, 0.1 mmol) and CH₂O (13 mg, 0.4 mmol) in MeOH (10 ml) was stirred at 25° C. for 10 min. Then NaBH(AcO)₃ (55 mg, 0.2 mmol) was added. The reaction mixture was stirred at 25° C. for 1h. H₂O was added into mixture. The resulting mixture was extracted with EtOAc, the organic phase was successively washed with water and brine, dried over anhydrous Na₂SO₄, after filtration, the filtrate was concentrated under reduced pressure. The residue was purified by column chromatography on silica gel (PE/EtOAc=1/1) to afford 385-2 (30 mg, 53%) as a white solid. MS (ESI) m/z 592 [M+H]⁺.

Step 3 was performed according to the procedure outlined for step 2 for preparation of compound 7 to afford 385 as a white solid (12 mg, 31%). MS (ESI) m/z 462 [M+H]⁺. ¹H NMR (400 MHz, CD₃OD) δ 7.89 (d, J=8.0 Hz, 1H), 7.85 (d, J=8.8 Hz, 1H), 7.77 (s, 1H), 7.57-7.51 (m, 2H), 7.37 (d, J=8.6 Hz, 1H), 6.70 (s, 1H), 5.53 (d, J=15.3 Hz, 1H), 4.75 (dd, J=8.4, 6.3 Hz, 1H), 4.54 (d, J=15.4 Hz, 1H), 4.30-4.20 (m, 2H), 4.13-4.04 (m, 1H), 3.59 (dd, J=14.8, 6.3 Hz, 1H), 3.51 (s, 3H), 3.04-2.96 (m, 3H), 2.66 (s, 3H).

### (E)-3-((3-bromopyridin-2-yl)methyl)-2-(3-(5-methyl-1H-1,2,3-triazol-4-yl)allyl)isoindolin-1-one (386)

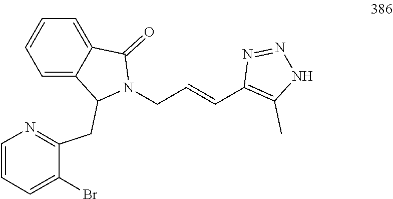 [SMILES: Cc1[nH]nnc1/C=C/CN1C(=O)c2ccccc2C1Cc1ncccc1Br]

The title compound 386 was prepared according to the procedure described for compound 328 as a white solid (2 mg, 4%). MS (ESI) m/z 424 [M+H]⁺. ¹H NMR (400 MHz, DMSO-d₆) δ 8.51 (dd, J=4.6, 1.5 Hz, 1H), 8.00 (dd, J=8.1, 1.5 Hz, 1H), 7.67-7.56 (m, 1H), 7.49-7.34 (m, 2H), 7.24 (dd, J=8.1, 4.6 Hz, 1H), 7.04-6.92 (m, 1H), 6.26 (d, J=16.1 Hz, 1H), 6.14-6.02 (m, 1H), 5.26 (t, J=6.6 Hz, 1H), 4.62-4.45 (m, 1H), 3.94 (dd, J=15.8, 7.1 Hz, 1H), 3.57 (dd, J=14.9, 6.0 Hz, 1H), 3.20-3.13 (m, 1H), 2.16 (s, 3H).

The intermediate 386-2 was prepared as follows:

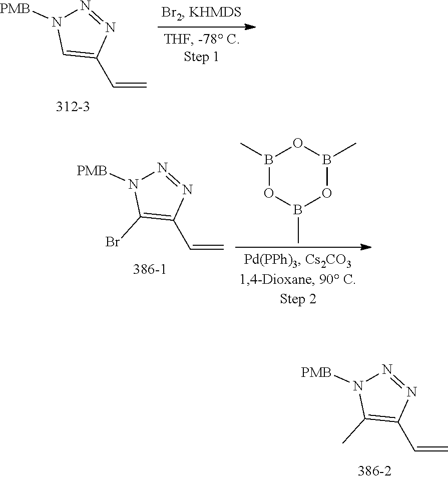

### Step 1. 5-bromo-1-(4-methoxybenzyl)-4-vinyl-1H-1,2,3-triazole (386-1)

To a solution of 312-3 (1.05 g, 4.88 mmol) in THF (20 mL) was added KHMDS (1.07 g, 5.37 mmol) and the mixture was stirred at −78° C. for 1 h. Then bromine (0.30 mL, 5.85 mmol) was added and the mixture was stirred for 30 min at −78° C. and 2 h at r.t. The reaction was monitored by LC/MS. Once completed, the mixture was diluted with DCM, washed with water, dried over Na₂SO₄, filtered and concentrated under reduced pressure. The residue was purified by column chromatograph (PE/EA=10/1) to yield 386-1 (240 mg, 17%). ¹H NMR (400 MHz, CDCl₃) δ 7.28-7.20 (m, 2H), 6.90-6.80 (m, 2H), 6.57 (dd, J=17.7, 11.3 Hz, 1H), 6.18 (dd, J=17.8, 1.4 Hz, 1H), 5.46 (s, 2H), 5.41 (dd, J=11.4, 1.4 Hz, 1H), 3.77 (s, 3H).

Step 2 was performed according to the procedure outlined for preparation of 26-1.

### 6-((1-((3-bromopyridin-2-yl)methyl)-3-oxoisoindolin-2-yl)methyl)oxazolo[4,5-c]pyridin-2(3H)-one (387)

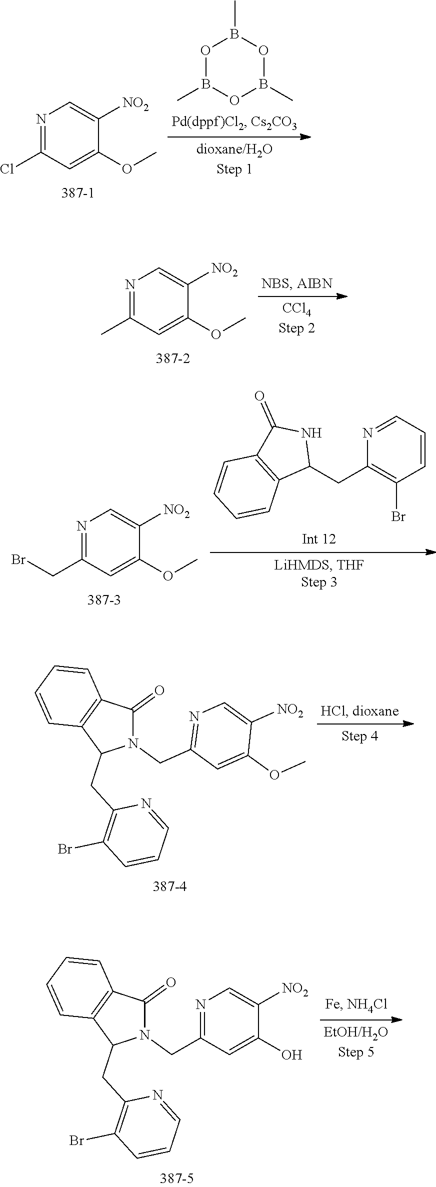

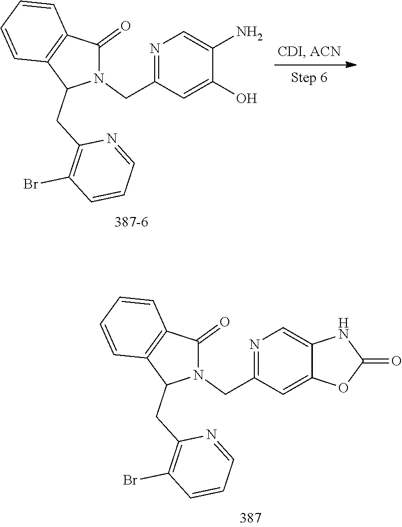 [SMILES: Nc1cnc(CN2C(=O)c3ccccc3C2Cc2ncccc2Br)cc1O ; O=C1c2ccccc2C(Cc2ncccc2Br)N1Cc1cc2oc(=O)[nH]c2cn1]

Step 1 was performed according to the procedure outlined for preparation of 26-1.

Step 2 was performed according to the procedure outlined for preparation of 222-1.

Step 3 was performed according to the procedure outlined for preparation of 222-2.

### Step 4. 3-((3-bromopyridin-2-yl)methyl)-2-((4-hydroxy-5-nitropyridin-2-yl) methyl)isoindolin-1-one (387-5)

A mixture of 387-4 (30 mg, 0.064 mmol) in dioxane (3 mL) and HCl (1 mL, 2M in water) was stirred at 90° C. overnight. The mixture was concentrated and purified by FCC (PE/EA=2/1) to give 387-5 (25 mg, 83%) as a yellow oil. MS (ESI) m/z 455 [M+H]⁺.

### Step 5. 2-((5-amino-4-hydroxypyridin-2-yl)methyl)-3-((3-bromopyridin-2-yl)methyl)isoindolin-1-one (387-6)

To a solution of 387-5 (25 mg, 0.05 mmol) in EtOH/H₂O (1 mL/0.1 mL) were added NH₄Cl (30 mg, 0.56 mmol) and Fe (30 mg, 0.54 mmol) at r.t. The mixture was stirred at 80° C. for 2 h. The mixture was concentrated and purified by FCC (PE/EA=1/1) to give 387-6 (20 mg, 85%) as a yellow oil. MS (ESI) m/z 425 [M+H]⁺.

Step 6 was performed according to the procedure outlined for preparation of Int 1-2 to afford 387 as a white solid (3 mg, 14%). MS (ESI) m/z 451 [M+H]⁺. ¹H NMR (400 MHz, DMSO-d₆) δ 8.50-8.49 (m, 1H), 8.09 (s, 1H), 8.00 (dd, J=8.4, 0.8 Hz, 1H), 7.73-7.71 (m, 1H), 7.52-7.48 (m, 2H), 7.24 (dd, J=8.0, 4.4 Hz, 1H), 7.08-7.05 (m, 2H), 5.32 (t, J=6.4 Hz, 1H), 5.04 (d, J=15.6 Hz, 1H), 4.52 (d, J=16.0 Hz, 1H), 3.59 (dd, J=14.8, 6.0 Hz, 1H), 3.22 (dd, J=15.2, 7.2 Hz, 1H).

### 2-((1H-benzo[d][1,2,3]triazol-5-yl)methyl)-3-((4-chloro-1H-pyrazol-3-yl)methyl)isoindolin-1-one (388)

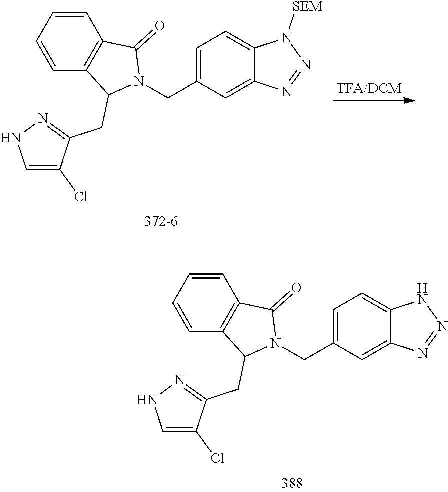 [SMILES: C[Si](C)(C)CCOCn1nnc2cc(CN3C(=O)c4ccccc4C3Cc3n[nH]cc3Cl)ccc21 ; O=C1c2ccccc2C(Cc2n[nH]cc2Cl)N1Cc1ccc2[nH]nnc2c1]

To a solution of 372-6 (70 mg, 0.14 mmol) in DCM (2 mL) was added TFA (2 mL). The mixture was stirred at r.t. overnight, and then concentrated under vacuum. The residue was purified by prep-HPLC to give 388 (16 mg, 30.2%) as a white solid. MS (ESI) m/z 379 [M+H]⁺. ¹H NMR (400 MHz, DMSO-d₆) δ 15.64 (s, 1H), 12.93 (s, 1H), 7.90-7.79 (m, 2H), 7.77-7.71 (m, 1H), 7.68 (d, J=7.4 Hz, 1H), 7.55-7.41 (m, 2H), 7.28 (d, J=9.1 Hz, 1H), 7.23-7.14 (m, 1H), 5.38-5.23 (m, 11H), 4.74 (t, J=5.7 Hz, 11H), 4.59 (d, J=15.6 Hz, 1H), 3.44-3.34 (m, 11H), 3.02 (dd, J=14.9, 6.8 Hz, 1H).

### 2-((1H-benzo[d][1,2,3]triazol-5-yl)methyl)-3-((4-chloro-1-(2-methoxyethyl)-1H-pyrazol-5-yl)methyl)isoindolin-1-one (389) and 2-((1H-benzo[d][1,2,3]triazol-5-yl)methyl)-3-((4-chloro-1-(2-methoxyethyl)-1H-pyrazol-3-yl)methyl)isoindolin-1-one (390)

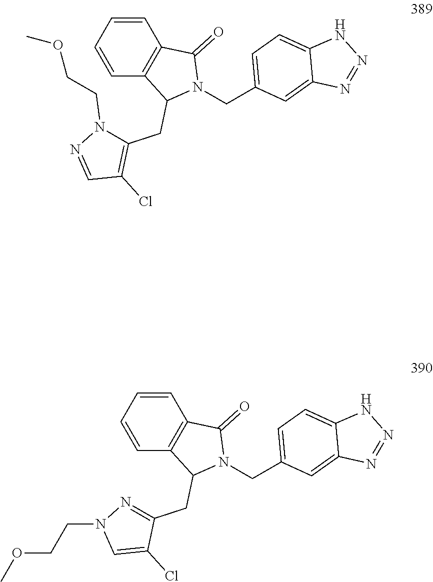 [SMILES: COCCn1ncc(Cl)c1CC1c2ccccc2C(=O)N1Cc1ccc2[nH]nnc2c1 ; COCCn1cc(Cl)c(CC2c3ccccc3C(=O)N2Cc2ccc3[nH]nnc3c2)n1]

The title compounds 389 and 390 were prepared according to the procedure outlined for compound 372. After SFC separation, two regioisomers were obtained: compound 389 and 390. One of the two isomers was a white solid (5 mg, 3.8%), with RT=2.32 min (CHIRAL SFC, column: CHIRALPAK AD-H; column size: 250 mm*20 mm, Sum; Modifier: 40% IPA (NH₄OH 0.2%); flow: 40 g/min). MS (ESI) m/z 437 [M+H]⁺. ¹H NMR (400 MHz, DMSO-d₆) δ 15.56 (s, 1H), 7.85 (d, J=8.5 Hz, 1H), 7.79-7.75 (m, 1H), 7.74 (s, 1H), 7.60 (s, 1H), 7.54-7.45 (m, 2H), 7.23 (d, J=8.3 Hz, 1H), 6.80-6.72 (m, 1H), 5.36 (d, J=15.2 Hz, 1H), 4.70 (dd, J=8.9, 5.8 Hz, 1H), 4.51 (d, J=15.3 Hz, 1H), 4.07-3.89 (m, 2H), 3.61-3.46 (m, 2H), 3.41-3.35 (m, 1H), 2.95 (s, 3H), 2.87 (dd, J=14.8, 9.0 Hz, 1H). The other one of the two isomers was a white solid (25 mg, 19.1%), with RT=3.95 min (CHIRAL SFC, column: CHIRALPAK AD-H; column size: 250 mm*20 mm, Sum; Modifier: 40% IPA (NH₄OH 0.2%); flow: 40 g/min). MS (ESI) m/z 437 [M+H]⁺. ¹H NMR (400 MHz, DMSO-d₆) δ 15.62 (s, 1H), 7.85 (d, J=8.5 Hz, 1H), 7.82 (s, 1H), 7.74-7.67 (m, 2H), 7.53-7.43 (m, 2H), 7.26 (dd, J=8.6, 1.5 Hz, 1H), 7.19 (dd, J=6.5, 1.8 Hz, 1H), 5.31 (d, J=15.4 Hz, 1H), 4.73 (dd, J=7.1, 4.9 Hz, 1H), 4.58 (d, J=15.4 Hz, 1H), 4.19-4.05 (m, 2H), 3.57 (t, J=5.2 Hz, 2H), 3.31 (dd, J=14.8, 5.0 Hz, 1H), 3.18 (s, 3H), 2.94 (dd, J=14.8, 7.2 Hz, 1H).

### 2-((1H-benzo[d][1,2,3]triazol-5-yl)methyl)-3-((4-chloro-1-cyclopropyl-1H-pyrazol-5-yl)methyl)isoindolin-1-one (391) and 2-((1H-benzo[d][1,2,3]triazol-5-yl)methyl)-3-((4-chloro-1-cyclopropyl-1H-pyrazol-3-yl)methyl)isoindolin-1-one (392)

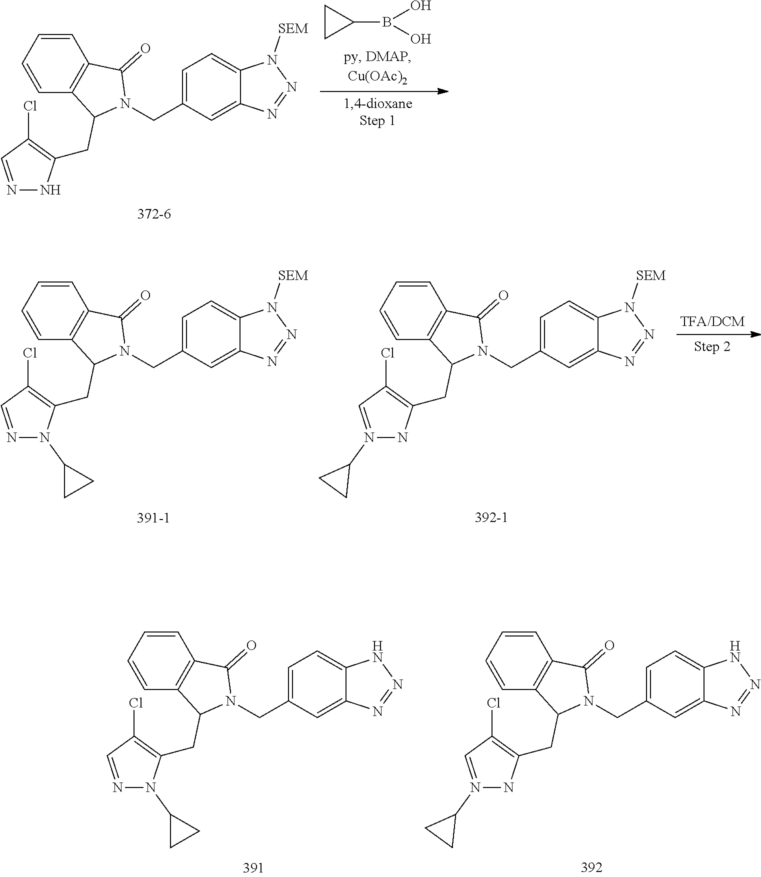

### Step 1. Mixture of 3-((4-chloro-1-cyclopropyl-1H-pyrazol-5-yl)methyl)-2-((1-((2-(trimethylsilyl)ethoxy)methyl)-1H-benzo[d][1,2,3]triazol-5-yl)methyl)isoindolin-1-one (391-1) and 3-((4-chloro-1-cyclopropyl-1H-pyrazol-3-yl)methyl)-2-((1-((2-(trimethylsilyl) ethoxy)methyl)-1H-benzo[d][1,2,3]triazol-5-yl)methyl)isoindolin-1-one (392-1)

To a solution of 372-6 (200 mg, 0.4 mmol) in dioxane (10 mL) were added cyclopropylboronic acid (102 mg, 1.2 mmol), pyridine (34 mg, 0.44 mmol), DMAP (144 mg, 1.2 mmol), cupric acetate monohydrate (102 mg, 0.52 mmol) under 02. The mixture was stirred at r.t. overnight. Water was added and then the mixture was extracted with EtOAc. The organic layer was separated, dried over anhydrous Na₂SO₄ and concentrated, the residue was purified by TLC to give a mixture of 391-1 and 392-1 (60 mg, 27.4%) as a yellow oil. MS (ESI) m/z 549 [M+H]⁺.

Step 2 was performed according to the procedure outlined for step 2 for the preparation of compound 7. After SFC separation, two regioisomers were obtained: compound 391 and 392. One of the two isomers was a white solid (3 mg, 12%), with RT=3.06 min (CHIRAL SFC, column: CHIRALPAK AD-H; column size: 250 mm*20 mm, 5 μm; Modifier: 40% IPA (NH₄OH 0.2%); flow: 40 g/min). MS (ESI) m/z 419 [M+H]⁺. ¹H NMR (400 MHz, CDCl₃) δ 7.95 (d, J=7.5 Hz, 1H), 7.87 (d, J=7.7 Hz, 1H), 7.72-7.59 (m, 1H), 7.56-7.42 (m, 3H), 7.32 (d, J=8.6 Hz, 1H), 6.73 (d, J=7.4 Hz, 1H), 5.57 (d, J=15.2 Hz, 1H), 4.83 (t, J=7.6 Hz, 1H), 4.34 (d, J=15.2 Hz, 1H), 3.45 (dd, J=14.5, 6.7 Hz, 1H), 3.01 (dd, J=14.5, 8.5 Hz, 1H), 2.91-2.82 (m, 1H), 1.34-1.20 (m, 2H), 1.04-0.90 (m, 2H). The other one of the two isomers was a white solid (29 mg, 53.4%), with RT=4.48 min (CHIRAL SFC, column: CHIRALPAK AD-H; column size: 250 mm*20 mm, 5 μm; Modifier: 40% IPA (NH₄OH 0.2%); flow: 40 g/min). MS (ESI) m/z 419 [M+H]⁺. ¹H NMR (400 MHz, DMSO-d₆) δ 7.88 (s, 1H), 7.86 (d, J=8.6 Hz, 1H), 7.73-7.62 (m, 2H), 7.56-7.41 (m, 2H), 7.30-7.18 (m, 2H), 5.29 (d, J=15.5 Hz, 1H), 4.74 (t, J=6.6 Hz, 1H), 4.57 (d, J=15.5 Hz, 1H), 3.66-3.53 (m, 1H), 3.26 (dd, J=14.9, 4.9 Hz, 1H), 2.99 (dd, J=14.9, 6.6 Hz, 1H), 0.99-0.78 (m, 4H).

### 4-((2-((1H-benzo[d][1,2,3]triazol-5-yl)methyl)-3-oxoisoindolin-1-yl)methyl)-1,5-dimethyl-1H-pyrazole-3-carbonitrile (393)

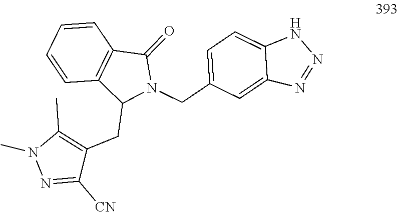 [SMILES: Cc1c(CC2c3ccccc3C(=O)N2Cc2ccc3[nH]nnc3c2)c(C#N)nn1C]

The title compound 393 was prepared according to the procedure described for compound 279 as a white solid (30 mg, 30%). MS (ESI) m/z 398 [M+H]⁺. ¹H NMR (400 MHz, CDCl₃) δ 7.92-7.86 (m, 2H), 7.81 (s, 1H), 7.50-7.43 (m, 2H), 7.38 (d, J=7.9 Hz, 1H), 7.02-6.93 (m, 1H), 5.51 (d, J=15.2 Hz, 1H), 4.62 (dd, J=8.2, 5.0 Hz, 1H), 4.57 (d, J=15.2 Hz, 1H), 3.82 (s, 3H), 3.28 (dd, J=14.8, 4.8 Hz, 1H), 2.76 (dd, J=14.7, 8.1 Hz, 1H), 1.85 (s, 3H).

The intermediate 393-6 was prepared as follows:

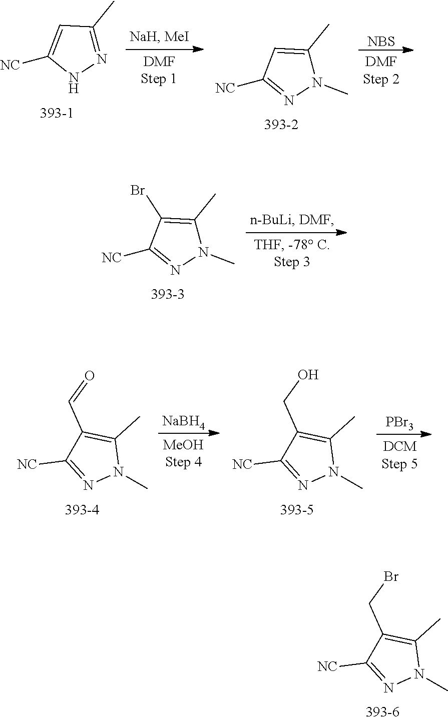

### Step 1: 1,5-dimethyl-1H-pyrazole-3-carbonitrile (393-2)

To a solution of 3-methyl-1H-pyrazole-5-carbonitrile (1 g, 9.3 mmol) in dried DMF (20 mL) was added NaH (247 mg, 10.2 mol) in portions at 0° C. under N₂. The mixture was stirred for 30 min, and then added dropwise Mel (2.2 g, 15 mmol). The mixture was stirred at 0° C. for another 2 h, then was quenched with water (20 mL) and extracted with ethyl acetate (50 mL*2). The combined organic layer was washed with water (50 mL) and brine (50 mL), dried over Na₂SO₄, filtered, and concentrated in vacuo. The residue was purified by column chromatography on silica gel (eluting with PE/EA=2/1 to 1/1) to give 393-2 (600 mg, 53%) as a yellow solid. MS (ESI) m/z 122 [M+H]⁺.

### Step 2: 4-bromo-1,5-dimethyl-1H-pyrazole-3-carbonitrile (393-3)

A mixture of 393-2 (500 mg, 4.1 mmol) and NBS (875 mg, 4.9 mmol) in dried DMF (10 mL) was stirred at 55° C. for 16 h. The mixture was extracted with ethyl acetate (20 mL*3), The combined organic layer was washed with water (30 mL) and brine (30 mL), dried over Na₂SO₄, filtered, and concentrated in vacuo. The residue was purified by column chromatography on silica gel (eluting with PE/EA=3/1 to 2/1) to give 393-3 (620 mg, 76%) as white solid. MS (ESI) m/z 200 [M+H]⁺.

### Step 3: 4-formyl-1,5-dimethyl-1H-pyrazole-3-carbonitrile (393-4)

To a solution of 393-3 (620 mg, 3.1 mmol) in dried THF (5 mL) was added dropwise n-BuLi (1.55 mL, 3.7 mmol) at −78° C. under N₂. After 1 h, then added DMF (566 mg, 7.75 mmol). The mixture was stirred for 1 h at −78° C.T, quenched with NH₄Cl (aq.) (10 mL) and extracted with ethyl acetate (2*30 mL). The combined organic layer was dried over anhydrous Na₂SO₄ and concentrated. The residue was purified by column chromatography on silica gel (eluting with PE/EA=2/1 to 1/1) to give 393-4 (430 mg, 93%) as a yellow oil. MS (ESI) m/z 150 [M+H]⁺.

Step 4 was performed according to the procedure outlined for preparation of 164-2.

Step 5 was performed according to the procedure outlined for preparation of 263-3.

### 2-((1H-benzo[d][1,2,3]triazol-5-yl)methyl)-3-((4-chloro-1-methyl-1H-pyrazol-5-yl)methyl)-5-(oxetan-3-ylmethyl)isoindolin-1-one (394)

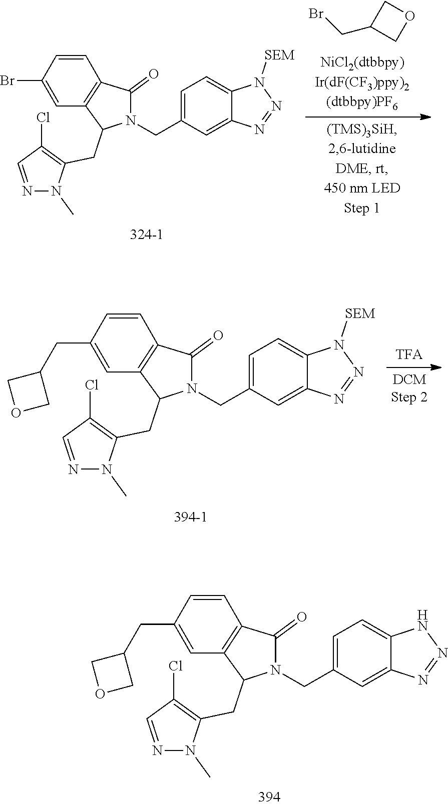 [SMILES: BrCC1COC1 ; Cn1ncc(Cl)c1CC1c2cc(Br)ccc2C(=O)N1Cc1ccc2c(c1)nnn2COCC[Si](C)(C)C ; Cn1ncc(Cl)c1CC1c2cc(CC3COC3)ccc2C(=O)N1Cc1ccc2c(c1)nnn2COCC[Si](C)(C)C ; Cn1ncc(Cl)c1CC1c2cc(CC3COC3)ccc2C(=O)N1Cc1ccc2[nH]nnc2c1]

Step 1 was performed according to the procedure outlined for preparation of 198-1.

Step 2 was performed according to the procedure outlined for preparation of compound 7 to afford 394 as a white solid (6 mg, 7%). MS (ESI) m/z 463[M+H]⁺. ¹H NMR (400 MHz, CD₃OD) δ 8.46 (s, 1H), 7.85 (d, J=7.9 Hz, 1H), 7.81-7.73 (m, 2H), 7.51 (s, 1H), 7.37 (t, J=8.6 Hz, 2H), 6.51 (s, 1H), 5.51 (d, J=15.2 Hz, 1H), 4.76-4.69 (m, 3H), 4.53 (d, J=15.5 Hz, 1H), 4.40 (t, J=6.0 Hz, 2H), 3.56 (dd, J=14.9, 6.2 Hz, 1H), 3.50 (s, 3H), 3.29-3.17 (m, 1H), 3.02 (d, J=7.7 Hz, 2H), 2.93 (dd, J=15.1, 8.4 Hz, 1H).

### 2-((1-((3-bromopyridin-2-yl)methyl)-3-oxoisoindolin-2-yl)methyl)-8-methyl-5-oxa-7-azaspiro[3.4]octan-6-one (395)

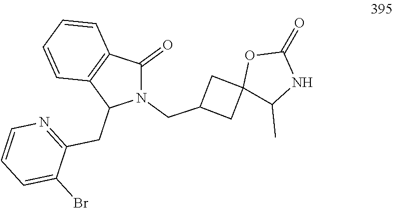 [SMILES: CC1NC(=O)OC12CC(CN1C(=O)c3ccccc3C1Cc1ncccc1Br)C2]

The title compound 395 was prepared according to the procedure described for compound 270 as a white solid (57 mg, 33.5%). MS (ESI) m/z 456 [M+H]⁺. ¹H NMR (400 MHz, DMSO-d₆) δ 8.61 (d, J=4.0 Hz, 1H), 8.10 (dd, J=8.0, 1.2 Hz, 1H), 7.68-7.66 (m, 1H), 7.54-7.44 (m, 3H), 7.32 (dd, J=8.4, 4.8 Hz, 1H), 7.03-7.00 (m, 1H), 5.30-5.25 (m, 1H), 3.97-3.92 (m, 1H), 3.70-3.61 (m, 2H), 3.30-3.25 (m, 1H), 3.21-3.14 (m, 1H), 2.47-2.35 (m, 1H), 2.31-2.19 (m, 1H), 2.16-2.01 (m, 2H), 1.98-1.90 (m, 1H), 1.08 (t, J=6.0 Hz, 3H).

### 2-((1H-benzo[d][1,2,3]triazol-5-yl)methyl)-3-((4-chloro-1-methyl-1H-pyrazol-5-yl)methyl)-5-(2-methoxypropan-2-yl)isoindolin-1-one (396)

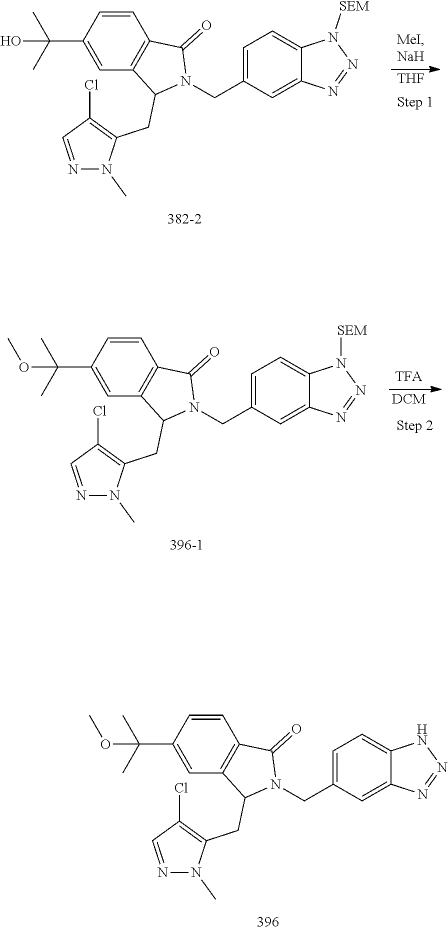

Step 1 was performed according to the procedure outlined for preparation of 393-2.

Step 2 was performed according to the procedure outlined for preparation of compound 7 to afford 396 as white solid (13 mg, 20%). MS (ESI) m/z 465 [M+H]⁺. ¹H NMR (400 MHz, CDCl₃) δ 7.91 (d, J=8.0 Hz, 1H), 7.88-7.85 (m, 1H), 7.73 (s, 1H), 7.59 (dd, J=8.1, 1.1 Hz, 1H), 7.50 (s, 1H), 7.34 (d, J=8.5 Hz, 1H), 6.66 (s, 1H), 5.54 (d, J=15.2 Hz, 1H), 4.69 (d, J=8.7, 6.5 Hz, 1H), 4.37 (d, J=15.3 Hz, 1H), 3.46 (s, 3H), 3.42 (d, J=14.5, 6.5 Hz, 1H), 3.03 (s, 3H), 2.78 (dd, J=14.5, 8.8 Hz, 1H), 1.45 (s, 6H), 1.42 (s, 6H).

### 2-((1H-benzo[d][1,2,3]triazol-5-yl)methyl)-3-((4-chloro-3-(difluoromethoxy)-1-methyl-1H-pyrazol-5-yl)methyl)isoindolin-1-one (397)

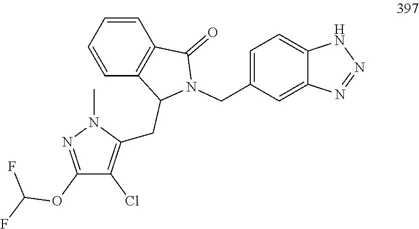 [SMILES: Cn1nc(OC(F)F)c(Cl)c1CC1c2ccccc2C(=O)N1Cc1ccc2[nH]nnc2c1]

The title compound 397 was prepared according to the procedure described for compound 279 as a white solid (4 mg, 11%). MS (ESI) m/z 459 [M+H]⁺. i H NMR (400 MHz, CDCl₃) δ 12.64 (s, 1H), 7.95 (d, J=6.7 Hz, 1H), 7.88 (d, J=8.2 Hz, 1H), 7.73 (s, 1H), 7.55-7.45 (m, 2H), 7.34 (d, J=8.5 Hz, 1H), 6.90 (s, 1H), 6.77 (d, J=6.6 Hz, 1H), 5.51 (d, J=15.3 Hz, 1H), 4.73 (dd, J=8.5, 6.3 Hz, 1H), 4.43 (d, J=15.3 Hz, 1H), 3.36 (s, 3H), 3.35 (dd, J=14.2, 6.2 Hz, 1H), 2.77 (dd, J=14.7, 8.7 Hz, 1H).

### 2-((1H-benzo[d][1,2,3]triazol-5-yl)methyl)-3-((4-chloro-1-methyl-1H-pyrazol-5-yl)methyl)-5-(tetrahydro-2H-pyran-4-yl)isoindolin-1-one (398)

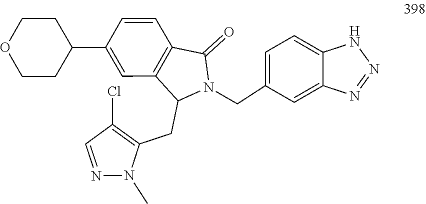 [SMILES: Cn1ncc(Cl)c1CC1c2cc(C3CCOCC3)ccc2C(=O)N1Cc1ccc2[nH]nnc2c1]

The title compound 398 was prepared according to the procedure described for compound 279 as a white solid (20 mg, 17%). MS (EST) m/z 477 [M+H]⁺. ¹H NMR (400 MHz, CDCl₃) δ 7.88 (d, J=7.9 Hz, 2H), 7.78 (s, 1H), 7.50 (s, 1H), 7.37-7.31 (m, 2H), 6.44 (s, 1H), 5.53 (d, J=15.2 Hz, 1H), 4.69 (dd, J=8.8, 6.3 Hz, 1H), 4.42 (d, J=15.2 Hz, 1H), 4.11-3.98 (m, 2H), 3.54-3.42 (m, 3H), 3.45 (s, 3H), 2.80-2.65 (m, 2H), 1.75-1.60 (m, 4H).

The intermediate 398-2 was prepared as follows:

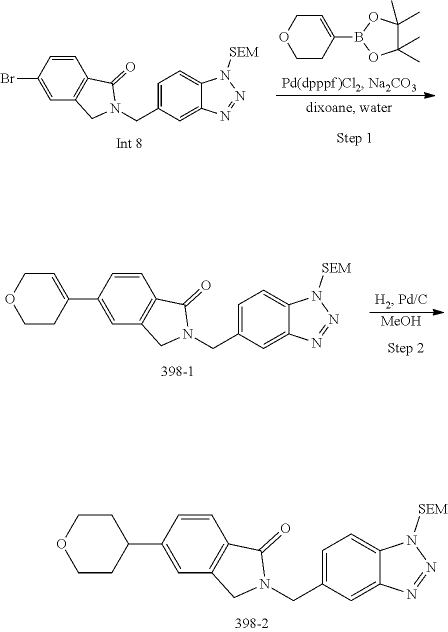 [SMILES: "MeOH" ; C[Si](C)(C)CCOCn1nnc2cc(CN3Cc4cc(Br)ccc4C3=O)ccc21 ; CC1(C)OB(C2=CCOCC2)OC1(C)C ; C[Si](C)(C)CCOCn1nnc2cc(CN3Cc4cc(C5=CCOCC5)ccc4C3=O)ccc21 ; C[Si](C)(C)CCOCn1nnc2cc(CN3Cc4cc(C5CCOCC5)ccc4C3=O)ccc21]

Step 1 was performed according to the procedure outlined for preparation of 26-1.

Step 2 was performed according to the procedure outlined for preparation of 181-4.

### 2-((1H-benzo[d][1,2,3]triazol-5-yl)methyl)-3-((4-chloro-3-cyclopropyl-1-methyl-1H-pyrazol-5-yl)methyl)isoindolin-1-one (399)

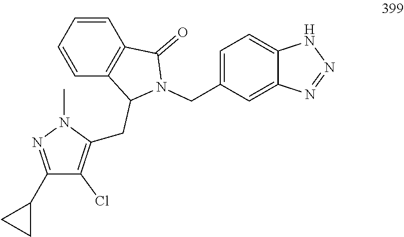 [SMILES: Cn1nc(C2CC2)c(Cl)c1CC1c2ccccc2C(=O)N1Cc1ccc2[nH]nnc2c1]

The title compound 399 was prepared according to the procedure described for compound 279 as a white solid (8 mg, 11%). MS (ESI) m/z 433 [M+H]⁺. ¹H NMR (400 MHz, CD₃OD) δ 7.94-7.83 (m, 2H), 7.75 (s, 1H), 7.60-7.50 (m, 2H), 7.33 (dd, J=8.6, 1.5 Hz, 1H), 6.90-6.82 (m, 1H), 5.52 (d, J=15.4 Hz, 1H), 4.75 (dd, J=8.3, 6.3 Hz, 1H), 4.49 (d, J=15.4 Hz, 1H), 3.47 (dd, J=14.8, 6.4 Hz, 1H), 3.42 (s, 3H), 2.94 (dd, J=14.8, 8.3 Hz, 1H), 1.93-1.83 (m, 1H), 1.00-0.80 (m, 4H).

The intermediate 399-4 was prepared as follows:

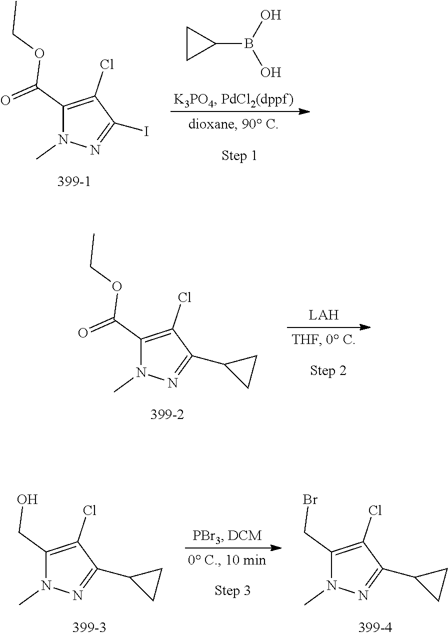

Step 1 was performed according to the procedure outlined for preparation of 26-1.

Step 2 was performed according to the procedure outlined for preparation of 263-2.

Step 3 was performed according to the procedure outlined for preparation of 263-3.

### 6-((1-((3-bromopyridin-2-yl)methyl)-3-oxoisoindolin-2-yl)methyl)-4,5,6,7-tetrahydrobenzo[d]oxazol-2(3H)-one (400)

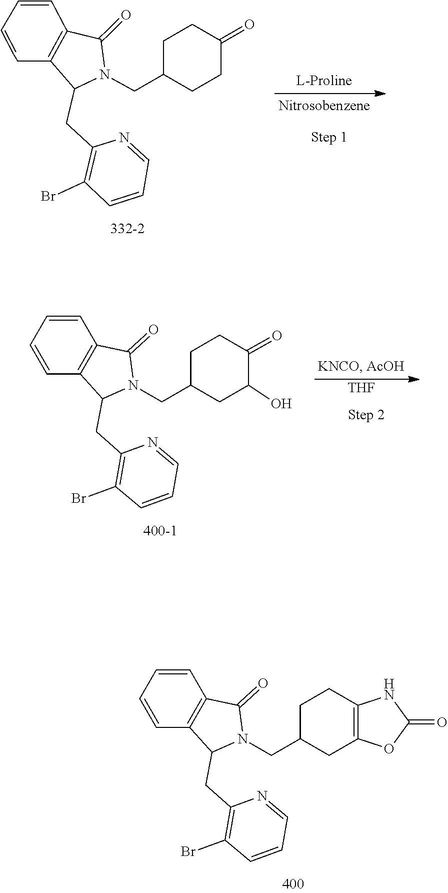 [SMILES: O=C1CCC(CN2C(=O)C3=C(CC=CC=C3)C2Cc2ncccc2Br)CC1 ; O=C1CCC(CN2C(=O)c3ccccc3C2Cc2ncccc2Br)CC1O ; O=C1c2ccccc2C(Cc2ncccc2Br)N1CC1CCc2[nH]c(=O)oc2C1 ; "THF"]

### Step 1. 3-((3-bromopyridin-2-yl)methyl)-2-((3-hydroxy-4-oxocyclohexyl)methyl)isoindolin-1-one (400-1)

To a solution of 332-2 (249 mg, 0.60 mmol) and L-Proline (14 mg, 0.12 mmol) in DCM (1.5 mL) was added PhNO (194 mg, 1.81 mmol) and the reaction mixture was stirred at 4° C. for 24 h. Then the reaction mixture was directly loaded on silica gel column without aqueous work-up. The mixture was purified by flash column chromatography to give 400-1 (100 mg, 38.7%) as a colorless oil. MS (ESI) m/z 429 [M+H]⁺.

### Step 2. 6-((1-((3-bromopyridin-2-yl)methyl)-3-oxoisoindolin-2-yl)methyl)-4,5,6,7-tetrahydrobenzo[d]oxazol-2(3H)-one (400)

To a stirred solution of the 400-1 (100 mg, 0.23 mmol) and KOCN (38 mg, 0.47 mmol) in THF (1 mL) at 50° C. was slowly added AcOH (0.032 mL, 0.56 mmol). The reaction mixture was stirred at this temperature for 4 h. After cooled to r.t, the reaction mixture was added water and extracted with EtOAc. The combined organic layer was washed with brine and dried over Na₂SO₄, and solvent was evaporated under reduced pressure. The residue was purified by HPLC to provide the compound 400 (30 mg, 28.4%) as a white solid. MS (ESI) m/z 454 [M+H]⁺. ¹H NMR (400 MHz, DMSO-d₆) δ 10.35 (s, 1H), 8.63-8.56 (m, 1H), 8.10 (d, J=8.1 Hz, 1H), 7.71-7.65 (m, 1H), 7.59-7.44 (m, 2H), 7.31 (dd, J=8.1, 4.6 Hz, 1H), 7.13 (t-like, J=6.2 Hz, 1H), 5.37-5.31 (m, 1H), 3.85 (dd, J=14.1, 9.3 Hz, 1H), 3.63-3.55 (m, 1H), 3.29-3.23 (m, 1H), 3.11 (dd, J=14.0, 4.7 Hz, 1H), 2.33-1.95 (m, 5H), 1.67-1.60 (m, 11H), 1.47-1.15 (m, 1H).

### (R)-3-((4-chloro-1-methyl-1H-pyrazol-5-yl)methyl)-2-(((R)-4,5,6,7-tetrahydro-1H-benzo[d][1,2,3]triazol-5-yl)methyl)isoindolin-1-one (401-1), (R)-3-((4-chloro-1-methyl-1H-pyrazol-5-yl)methyl)-2-(((S)-4,5,6,7-tetrahydro-1H-benzo[d][1,2,3]triazol-5-yl)methyl)isoindolin-1-one (401-2), (S)-3-((4-chloro-1-methyl-1H-pyrazol-5-yl)methyl)-2-(((S)-4,5,6,7-tetrahydro-1H-benzo[d][1,2,3]triazol-5-yl)methyl)isoindolin-1-one (402-1) and (S)-3-((4-chloro-1-methyl-1H-pyrazol-5-yl)methyl)-2-(((R)-4,5,6,7-tetrahydro-1H-benzo[d][1,2,3]triazol-5-yl)methyl)isoindolin-1-one (402-2)

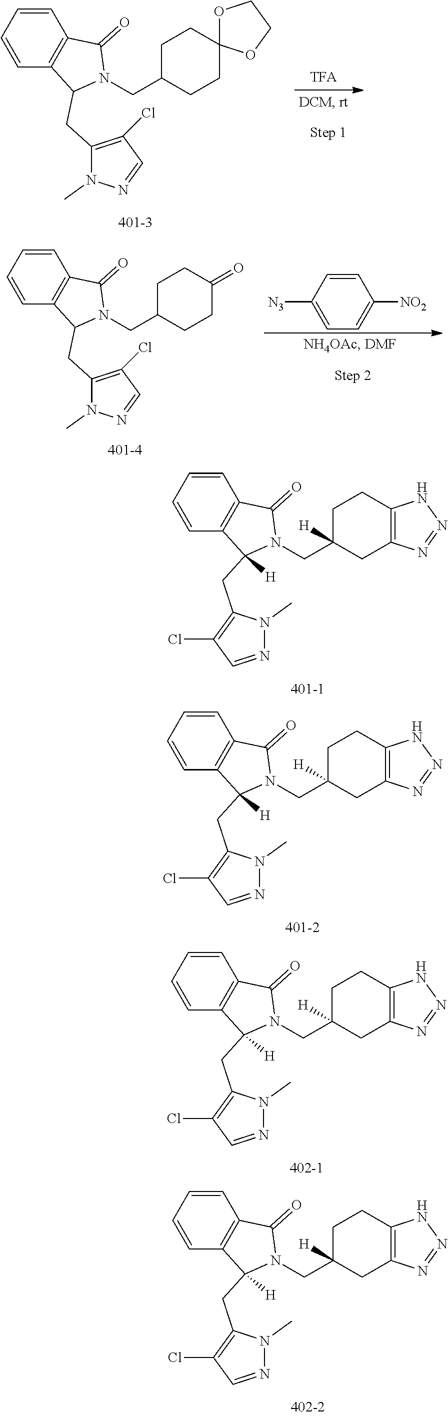

The intermediate 401-3 was prepared according to the procedure described for 307-2.

### Step 1. 3-((4-chloro-1-methyl-1H-pyrazol-5-yl)methyl)-2-((4-oxocyclohexyl) methyl)isoindolin-1-one (401-4)

To a solution of 401-3 (277 mg, 0.66 mmol) in THF/H₂O (2 mL/4 mL) was added TFA (4 mL) at r.t. The reaction mixture was stirred at r.t. for 2 h. The mixture was concentrated, diluted with H₂O (5 mL), adjusted to pH at 8 with sat. NaHCO₃, extracted with EA (10 mL*2), washed with brine (20 mL), dried over anhydrous Na₂SO₄, filtered and concentrated. The residue was purified by FCC (PE/EA=1/1) to give 401-4 as a yellow oil (300 mg, 99%). MS (ESI) m/z 372 [M+H]⁺.

### Step 2. (R)-3-((4-chloro-1-methyl-1H-pyrazol-5-yl)methyl)-2-(((R)-4,5,6,7-tetrahydro-1H-benzo[d][1,2,3]triazol-5-yl)methyl)isoindolin-1-one (401-1), (R)-3-((4-chloro-1-methyl-1H-pyrazol-5-yl)methyl)-2-(((S)-4,5,6,7-tetrahydro-1H-benzo[d][1,2,3]triazol-5-yl)methyl)isoindolin-1-one (401-2), (S)-3-((4-chloro-1-methyl-1H-pyrazol-5-yl)methyl)-2-(((S)-4,5,6,7-tetrahydro-1H-benzo[d][1,2,3]triazol-5-yl)methyl)isoindolin-1-one (402-1) and (S)-3-((4-chloro-1-methyl-1H-pyrazol-5-yl)methyl)-2-(((R)-4,5,6,7-tetrahydro-1H-benzo[d][1,2,3]triazol-5-yl)methyl)isoindolin-1-one (402-2)

A mixture of 401-2 (256 mg, 0.69 mmol), 1-azido-4-nitrobenzene (171 mg, 1.04 mmol), NH₄0Ac (266 mg, 3.45 mmol) in DMF (10 mL) was stirred at 80° C. overnight. Water (30 mL) was added and extracted with EA (20 mL*2), washed with water (20 mL*2) and brine (20 mL), dried over anhydrous Na₂SO₄, filtered, concentrated and purified by FCC (DCM/MeOH=20/1) to give a crude product. The crude product residue was purified by chiral HPLC (IA, 4.6 mm*250 mm 5 μm, Hex/EtOH=40/60, 1 ml/min) to give two products. One of the two products (containing two isomers of 401-1, 401-2, 402-1 and 402-2) was a yellow solid (46 mg, 17%), with RT=5.504 min, MS (ESI) m/z 397 [M+H]⁺. ¹H NMR (400 MHz, CD₃OD) δ 7.84-7.81 (m, 1H), 7.58-7.51 (m, 3H), 6.92-6.87 (m, 1H), 5.13-5.08 (m, 1H), 4.16-4.06 (m, 1H), 3.64-3.56 (m, 4H), 3.42-3.34 (m, 1H), 3.01-2.60 (m, 4H), 2.54-2.31 (m, 2H), 2.09-1.89 (m, 1H), 1.72-1.51 (m, 1H). The other one of the two products (containing the other two isomers of 401-1, 401-2, 402-1 and 402-2) was a yellow solid (49 mg, 18%), with RT=7.695 min, MS (ESI) m/z 397 [M+H]⁺. ¹H NMR (400 MHz, CD₃OD) δ 7.83-7.82 (m, 1H), 7.57-7.51 (m, 3H), 6.91-6.88 (m, 1H), 5.13-5.07 (m, 1H), 4.14-4.06 (m, 1H), 3.62-3.57 (m, 4H), 3.40-3.36 (m, 1H), 3.01-2.60 (m, 4H), 2.54-2.33 (m, 2H), 2.09-1.92 (m, 1H), 1.68-1.53 (m, 1H).

### 2-((1r,3r)-3-(1H-1,2,3-triazol-4-yl)cyclobutyl)-3-((3-bromopyridin-2-yl)methyl)isoindolin-1-one (403)

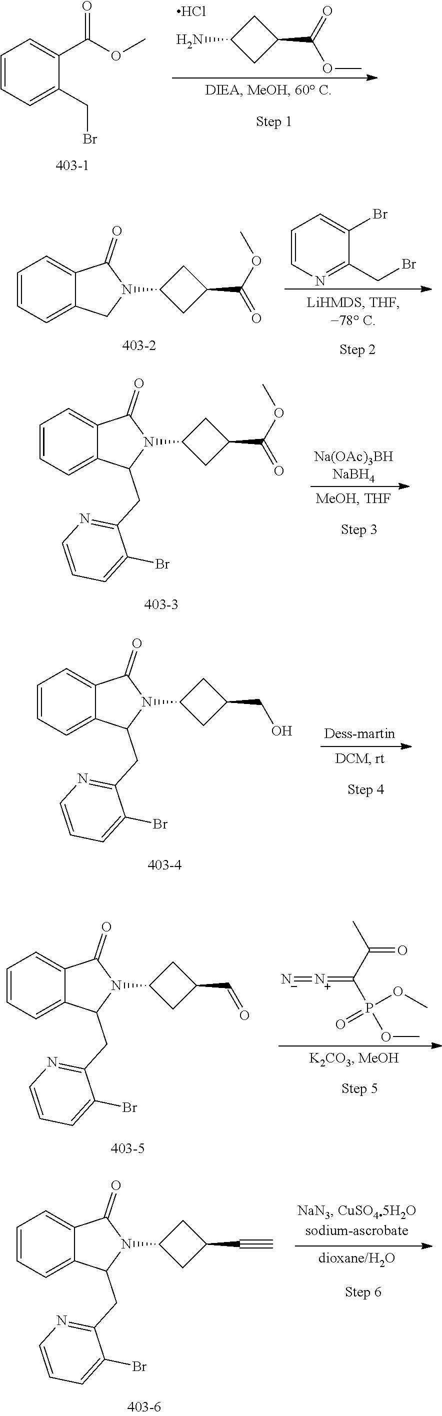

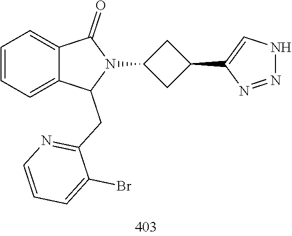 [SMILES: O=C1c2ccccc2C(Cc2ncccc2Br)N1[C@H]1C[C@H](c2c[nH]nn2)C1]

### Step 1: methyl (1r,3r)-3-(1-oxoisoindolin-2-yl)cyclobutane-1-carboxylate (403-2)

A mixture of 403-1 (500 mg, 2.18 mmol), HCl salt of methyl (1r,3r)-3-aminocyclobutane-1-carboxylate (435 mg, 2.62 mmol) and DIEA (703 mg, 5.45 mmol) in MeOH (20 mL) was stirred at 60° C. for 2 h. Water (20 mL) was added and extracted with EA (20 mL*3), the combined organic phase was washed with brine (20 mL), dried over anhydrous Na₂SO₄, filtered and concentrated. The residue was purified by FCC (PE/EA=5/1) to give 403-2 as a white solid (320 mg, 60%). MS (ESI) m/z 246 [M+H]⁺.

Step 2 was performed according to the procedure outlined for preparation of Int 12-2.

### Step 3: 3-((3-bromopyridin-2-yl)methyl)-2-((1r,3r)-3-(hydroxymethyl)cyclobutyl) isoindolin-1-one (403-4)

To a solution of 403-3 (500 mg, 1.21 mmol) in THF (20 mL) were added MeOH (155 mg, 4.84 mmol), NaBH₄ (138 mg, 3.63 mmol) and NaBH(OAc)₃ (51 mg, 0.24 mmol) at 0° C. The mixture was stirred at r.t. for 4 h. The mixture was added water (20 mL) and extracted with EA (20 mL*3), washed with brine (50 mL), dried over anhydrous Na₂SO₄, filtered and concentrated. The residue was purified by FCC (PE/EA=2/1) to give 403-4 as a white oil (400 mg, 85%). MS (ESI) m/z 387 [M+H]⁺.

### Step 4. (1r,3r)-3-(1-((3-bromopyridin-2-yl)methyl)-3-oxoisoindolin-2-yl) cyclobutane-1-carbaldehyde (403-5)

To a solution of 403-4 (400 mg, 1.04 mmol) in DCM (20 mL) was added Dess-Martin (661 mg, 1.56 mmol) at 0° C.C. The mixture was stirred at r.t. for 1 h. The mixture was poured into sat. Na₂S₂O₃ (20 mL) and extracted with DCM (20 mL*3). The pH of the combined organic layers was adjusted to 8 with sat. Na₂CO₃ (aq.), dried over anhydrous Na₂SO₄, filtered and concentrated under vacuum. The residue was purified by FCC (PE/EA=1/1) to give 403-5 as a colorless oil (280 mg, 70%). MS (ESI) m/z 385 [M+H]⁺.

### Step 5: 3-((3-bromopyridin-2-yl)methyl)-2-((1r,3r)-3-ethynylcyclobutyl)isoindolin-1-one (403-6)

To a solution of 403-5 (280 mg, 0.73 mmol) and K₂CO₃ (201 mg, 1.46 mmol) in MeOH (15 mL) was added dimethyl (1-diazo-2-oxopropyl)phosphonate (170 mg, 0.88 mmol) at 0° C. The mixture was stirred at r.t. for 2 h. The mixture was added water (20 mL) and extracted with EA (20 mL*3), washed with brine (20 mL), dried over anhydrous Na₂SO₄, filtered and concentrated. The residue was purified by FCC (PE/EA=2/1) to give 403-6 as a colorless oil (160 mg, 58%). MS (ESI) m/z 381 [M+H]⁺.

### Step 6: 2-((1r,3r)-3-(1H-1,2,3-triazol-4-yl)cyclobutyl)-3-((3-bromopyridin-2-yl)methyl)isoindolin-1-one (403)

A mixture of 403-6 (100 mg, 0.26 mmol), NaN₃ (51 mg, 0.78 mmol), CuSO₄.5H₂O (65 mg, 0.26 mmol) and sodium-ascorbate (52 mg, 0.26 mmol) in dioxane/H₂O (10 mL/2 mL) was stirred at 100° C. for 6 h. The mixture was added water (10 mL) and extracted with EA (20 mL*3), washed with brine (20 mL), dried over anhydrous Na₂SO₄, filtered and concentrated. The residue was purified by prep-HPLC (TFA) to give 403 as a white solid (13 mg, 12.1%). MS (ESI) m/z 424 [M+H]⁺. ¹H NMR (400 MHz, DMSO-d₆) δ 8.59 (dd, J=4.8, 1.6 Hz, 1H), 8.08 (dd, J=8.0, 0.9 Hz, 1H), 7.72 (s, 1H), 7.66-7.64 (m, 1H), 7.46-7.43 (m, 2H), 7.30 (dd, J=8.0, 4.4 Hz, 1H), 6.90-6.88 (m, 1H), 5.36-5.32 (m, 1H), 4.53-4.45 (m, 1H), 3.63-3.55 (m, 2H), 3.22-3.08 (m, 4H), 2.50-2.29 (m, 1H).

### 3-((1-((3-bromopyridin-2-yl)methyl)-3-oxoisoindolin-2-yl)methyl)cyclobutane-1-carboxamide (404)

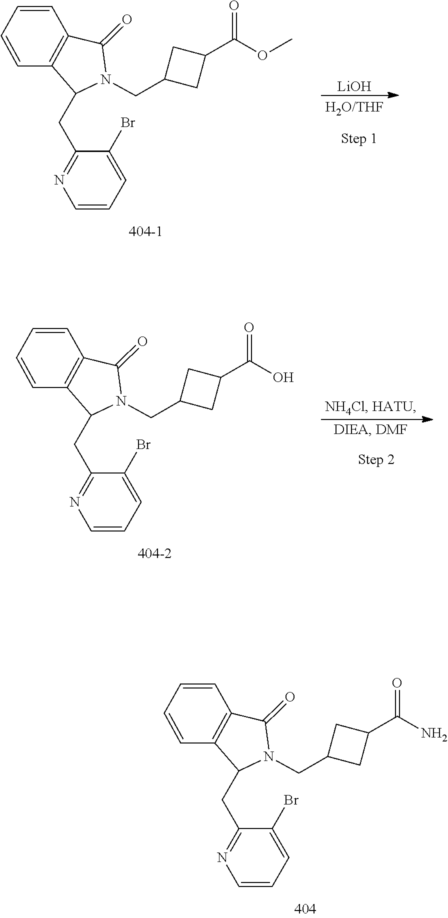 [SMILES: COC(=O)C1CC(CN2C(=O)c3ccccc3C2Cc2ncccc2Br)C1 ; "LiOH" ; O=C(O)C1CC(CN2C(=O)c3ccccc3C2Cc2ncccc2Br)C1 ; "DIEA, DMF" ; NC(=O)C1CC(CN2C(=O)c3ccccc3C2Cc2ncccc2Br)C1]

The intermediate 404-1 was prepared according to the procedure outlined for 222-2.

### Step 1. 3-((1-((3-bromopyridin-2-yl)methyl)-3-oxoisoindolin-2-yl)methyl) cyclobutane-1-carboxylic acid (404-2)

A mixture of 404-1 (50 mg, 0.17 mmol) and LiOH H₂O (15 mg, 0.34 mmol) in THF/H₂O (1 mL/1 mL) was stirred at r.t. for 16 h. The mixture was adjusted to pH at 5 with 0.1N HCl, extracted with EA (10 mL*2). The combined organic layers were dried over Na₂SO₄, filtered and concentrated to give 404-2 as a yellow oil (50 mg, 100%). MS (ESI) m/z 415 [M+H]⁺.

### Step 2. 3-((1-((3-bromopyridin-2-yl)methyl)-3-oxoisoindolin-2-yl)methyl) cyclobutane-1-carboxamide (404)

To a solution of 404-2 (50 mg, 0.12 mmol), NH₄Cl (48 mg, 0.12 mmol) and DIEA (46 mg, 0.36 mmol) in DMF (2 mL) was added HATU (48 mg, 0.12 mmol) in an ice-bath. The mixture was stirred at r.t. for 16 h. The mixture was concentrated and purified by prep-HPLC to give 404 as a white solid (21 mg, 40%). MS (ESI) m/z 414 [M+H]⁺. ¹H NMR (400 MHz, DMSO-d₆) δ 8.62-8.59 (m, 1H), 8.12-8.09 (m, 1H), 7.68-7.64 (m, 1H), 7.47-7.44 (m, 2H), 7.31 (dd, J=8.0, 4.8 Hz, 1H), 7.14-7.11 (m, 1H), 6.97-6.94 (m, 1H), 6.68 (s, 1H), 5.26-5.19 (m, 1H), 4.04 (dd, J=14.0, 8.8 Hz, 0.4H), 3.86 (dd, J=13.6, 7.6 Hz, 0.6H), 3.63 (dd, J=15.2, 5.2 Hz, 1H), 3.27-3.10 (m, 2H), 3.00-2.73 (m, 1H), 2.47-2.39 (m, 1H), 2.21-1.97 (m, 2H), 1.93-1.74 (m, 2H).

### (2s,4S)-2-(((R)-1-((4-chloro-1-methyl-1H-pyrazol-5-yl)methyl)-3-oxoisoindolin-2-yl)methyl)-5-oxa-7-azaspiro[3.4]octan-6-one (405) and (2s,4R)-2-(((S)-1-((4-chloro-1-methyl-1H-pyrazol-5-yl)methyl)-3-oxoisoindolin-2-yl)methyl)-5-oxa-7-azaspiro[3.4]octan-6-one (406)

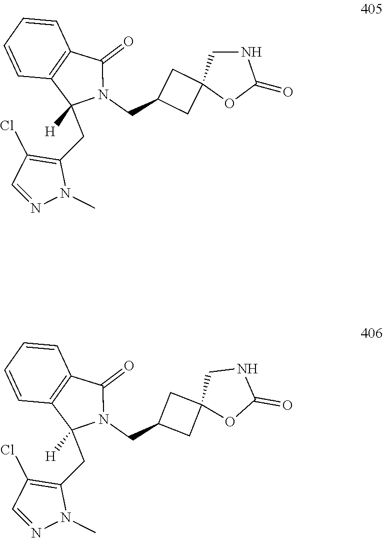 [SMILES: Cn1ncc(Cl)c1C[C@@H]1c2ccccc2C(=O)N1C[C@H]1C[C@]2(CNC(=O)O2)C1 ; Cn1ncc(Cl)c1C[C@H]1c2ccccc2C(=O)N1C[C@H]1C[C@]2(CNC(=O)O2)C1]

Chiral separation of compound 307 by chiral prep-HPLC (column: CHIRALPAK IA; column size: 2 cm×25 cm, 5 μm; mobile phase A: Hex:DCM; mobile phase B: EtOH) was conducted to afford two enantiomers: compound 405 and compound 406. 405 was a white solid, with RT=2.935 min (CHIRAL HPLC, column: CHIRALPAK IA-3; column size: 4.6*50 mm, 3 μm; mobile phase: (Hex:DCM=3:1) (0.1% DEA):EtOH=90:10; flow: 1.0 mL/min). MS (ESI) m/z 401 [M+H]⁺. ¹H NMR (400 MHz, CDCl₃) δ 7.86 (d, J=7.3 Hz, 1H), 7.53 (s, 1H), 7.48 (td, J=7.5, 1.1 Hz, 1H), 7.42 (td, J=7.5, 1.3 Hz, 1H), 6.54 (d, J=7.5 Hz, 1H), 5.10 (br, 1H), 4.84 (dd, J=9.8, 5.6 Hz, 1H), 4.22 (dd, J=14.4, 5.4 Hz, 1H), 3.61 (s, 2H), 3.49 (dd, J=14.4, 5.6 Hz, 1H), 3.46 (s, 3H), 3.20 (dd, J=14.4, 6.7 Hz, 1H), 2.59 (dd, J=14.4, 9.8 Hz, 1H), 2.49-2.31 (m, 5H). 406 was a white solid, with RT=4.923 min, (CHIRAL HPLC, column: CHIRALPAK IA-3; column size: 4.6*50 mm, 3 μm; mobile phase: (Hex:DCM=3:1)(0.10% DEA):EtOH=90:10; flow: 1.0 mL/min). MS (ESI) m/z 401 [M+H]⁺. ¹H NMR (400 MHz, CDCl₃) δ 7.86 (d, J=7.3 Hz, 1H), 7.53 (s, 1H), 7.48 (td, J=7.4, 1.1 Hz, 1H), 7.42 (td, J=7.5, 1.3 Hz, 1H), 6.54 (dd, J=7.5, 1.0 Hz, 1H), 5.08 (s, 1H), 4.85 (dd, J=9.8, 5.6 Hz, 1H), 4.22 (dd, J=14.3, 5.4 Hz, 1H), 3.61 (s, 2H), 3.50 (dd, J=14.6, 5.4 Hz, 1H), 3.46 (s, 3H), 3.20 (dd, J=14.4, 6.8 Hz, 1H), 2.59 (dd, J=14.4, 9.8 Hz, 1H), 2.50-2.29 (m, 5H). The structures of 405 and 406 were confirmed by X-ray.

### 4-methyl-3-(((R)-3-oxo-2-(((2s,4S)-6-oxo-5-oxa-7-azaspiro[3.4]octan-2-yl)methyl) isoindolin-1-yl)methyl)picolinonitrile (407) and 4-methyl-3-(((S)-3-oxo-2-(((2s,4R)-6-oxo-5-oxa-7-azaspiro[3.4]octan-2-yl)methyl)isoindolin-1-yl)methyl) picolinonitrile (408)

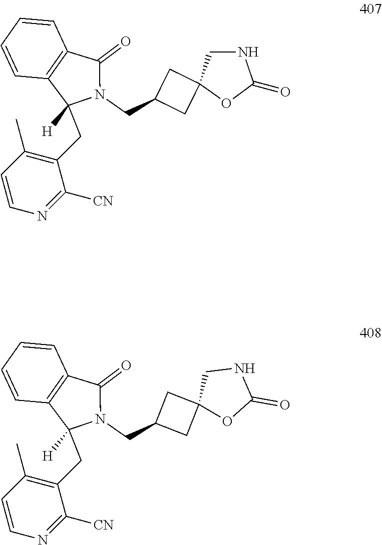 [SMILES: Cc1ccnc(C#N)c1C[C@@H]1c2ccccc2C(=O)N1C[C@H]1C[C@]2(CNC(=O)O2)C1 ; Cc1ccnc(C#N)c1C[C@H]1c2ccccc2C(=O)N1C[C@H]1C[C@]2(CNC(=O)O2)C1]

Chiral separation of compound 308 by chiral prep-HPLC (column: CHIRALPAK IA; column size: 2 cm×25 cm, 5 μm; mobile phase A: MTBE; mobile phase B: MeOH:DCM) was conducted to afford two enantiomers: compound 407 and compound 408. One of the two enantiomers was a white solid, with RT=2.628 min (CHIRAL HPLC, column: CHIRALPAK IA-3; column size: 4.6*50 mm, 3 μm; mobile phase: MtBE (0.1% DEA):(MeOH:DCM=1/1)=80:20; flow: 1.0 mL/min). MS (ESI) m/z 403 [M+H]⁺. The other one of the two enantiomers was a white solid, with RT=3.760 min, (CHIRAL HPLC, column: CHIRALPAK IA-3; column size: 4.6*50 mm, 3 μm; mobile phase: MtBE(0.1% DEA):(MeOH:DCM=1/1)=80:20; flow: 1.0 mL/min). MS (ESI) m/z 403 [M+H]⁺.

### (E)-3-((2-(3-(1H-1,2,3-triazol-4-yl)allyl)-3-oxoisoindolin-1-yl)methyl)-4-methylpicolinonitrile (409)

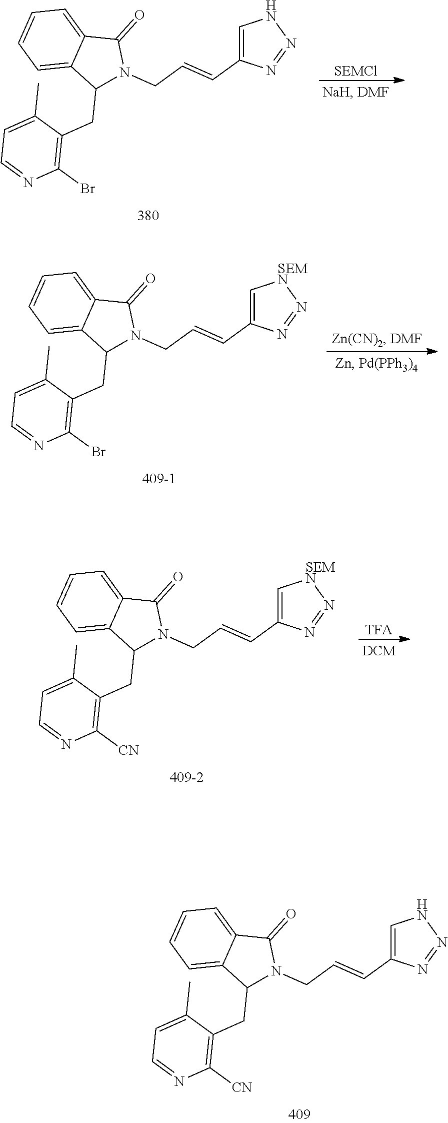

Step 1 was performed according to the procedure outlined for preparation of Int 4-2.

Step 2 was performed according to the procedure outlined for preparation of 284-2.

Step 3 was performed according to the procedure outlined for step 2 for the preparation of compound 7 to give 409 as a white solid (2 mg, 5.1%). MS (ESI) m/z 371 [M+H]⁺. ¹H NMR (400 MHz, DMSO-d₆) δ 14.93 (s, 1H), 8.58 (d, J=4.9 Hz, 1H), 7.96 (s, 1H), 7.77 (d, J=7.4 Hz, iH), 7.63 (d, J=5.0 Hz, 1H), 7.57-7.42 (m, 2H), 6.57 (d, J=16.1 Hz, 1H), 6.51 (d, J=7.5 Hz, 1H), 6.43-6.31 (m, 1H), 4.99 (dd, J=9.5, 5.9 Hz, 1H), 4.78-4.67 (m, 1H), 4.01 (dd, J=15.9, 7.6 Hz, 1H), 3.81 (dd, J=13.9, 6.0 Hz, 1H), 2.85 (dd, J=13.9, 9.5 Hz, 1H), 2.36 (s, 3H).

### 2-(3-((2-((1H-benzo[d][1,2,3]triazol-5-yl)methyl)-3-oxoisoindolin-1-yl)methyl)-4-chloro-1H-pyrazol-1-yl)acetonitrile (410)

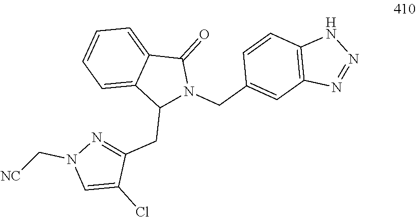 [SMILES: N#CCn1cc(Cl)c(CC2c3ccccc3C(=O)N2Cc2ccc3[nH]nnc3c2)n1]

The title compound 410 was prepared according to the procedure described for compound 373 as a white solid (9 mg, 10.8%). MS (ESI) m/z 418 [M+H]⁺. ¹H NMR (400 MHz, DMSO-d₆) δ 7.98 (s, 1H), 7.89-7.83 (m, 1H), 7.78-7.68 (m, 2H), 7.54-7.44 (m, 2H), 7.28 (d, J=8.4 Hz, 1H), 7.25-7.18 (m, 1H), 5.38 (s, 2H), 5.29 (d, J=15.5 Hz, 1H), 4.76 (t, J=6.1 Hz, 1H), 4.64 (d, J=15.5 Hz, 1H), 3.36 (dd, J=15.0, 5.0 Hz, 1H), 2.98 (dd, J=14.9, 7.4 Hz, 1H). CHIRAL SFC: (column: CHIRALPAK AD-H; column size: 250 mm*20 mm, 5 um; Modifier: 40% IPA (NH₄OH 0.2%); flow: 12.5 g/min) RT=2.11 min.

### 2-((1H-benzo[d][1,2,3]triazol-5-yl)methyl)-3-((4-chloro-1-(2-hydroxy-2-methylpropyl)-1H-pyrazol-3-yl)methyl)isoindolin-1-one (411)

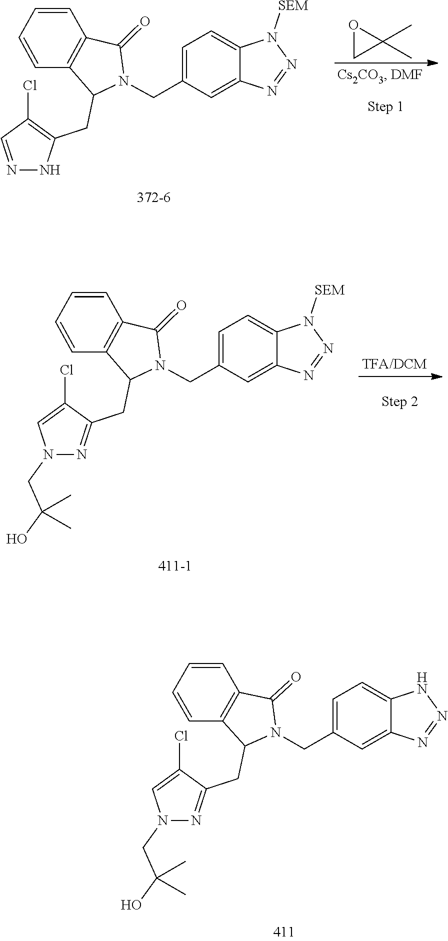

### Step 1. 3-((4-chloro-1-(2-hydroxy-2-methylpropyl)-1H-pyrazol-3-yl)methyl)-2-((1-((2-(trimethylsilyl)ethoxy)methyl)-1H-benzo[d][1,2,3]triazol-5-yl)methyl)isoindolin-1-one (411-1)

To a solution of 372-6 (150 mg, 0.3 mmol) in DMF (10 mL) were added 2,2-dimethyloxirane (64 mg, 0.9 mmol) and Cs₂CO₃ (192 mg, 0.6 mmol). The mixture was stirred for 8 h at 60′C. Water was added and the mixture was extracted with EtOAc. The organic layer was dried over anhydrous Na₂SO₄ and concentrated, and the residue was purified by TLC to give 411-1 (80 mg, 46%) as a yellow oil. MS (ESI) m/z 581 [M+H]⁺.

Step 2 was performed according to the procedure outlined for preparation of compound 7 to give 411 as a white solid (31 mg, 49.2%). MS (ESI) m/z 451 [M+H]⁺. ¹H NMR (400 MHz, DMSO-d₆) δ 15.59 (s, 1H), 7.87 (d, J=8.6 Hz, 1H), 7.77 (s, 1H), 7.69 (s, 1H), 7.65 (dd, J=6.7, 1.6 Hz, 1H), 7.53-7.40 (m, 2H), 7.34-7.24 (m, 2H), 5.32 (d, J=15.4 Hz, 1H), 4.73 (dd, J=6.8, 4.4 Hz, 1H), 4.63 (d, J=15.5 Hz, 1H), 3.92-3.80 (m, 2H), 3.39-3.31 (m, 1H), 3.02 (dd, J=14.8, 6.9 Hz, 1H), 0.94 (s, 3H), 0.90 (s, 3H). RT=4.13 min (HPLC).

### 2-((1H-benzo[d][1,2,3]triazol-5-yl)methyl)-3-((4-chloro-1-(2-oxaspiro[3.3]heptan-6-yl)-1H-pyrazol-3-yl)methyl)isoindolin-1-one (412)

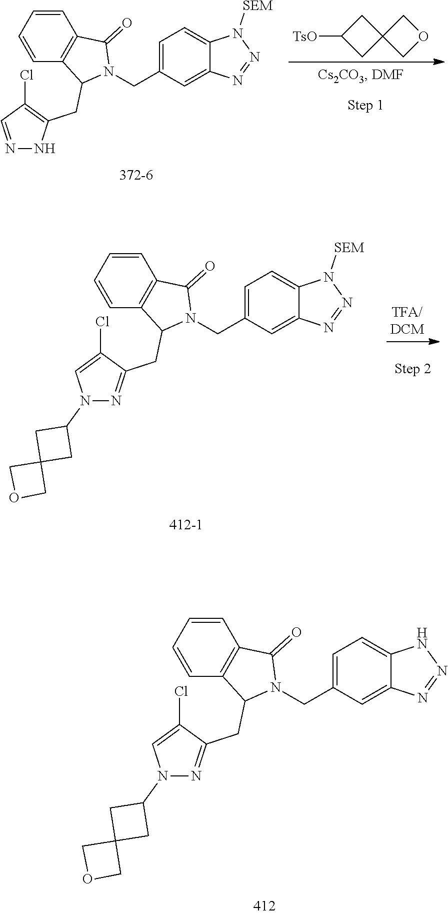 [SMILES: C[Si](C)(C)CCOCn1nnc2cc(CN3C(=O)c4ccccc4C3Cc3[nH]ncc3Cl)ccc21 ; Cc1ccc(S(=O)(=O)OC2CC3(COC3)C2)cc1 ; C[Si](C)(C)CCOCn1nnc2cc(CN3C(=O)c4ccccc4C3Cc3nn(C4CC5(COC5)C4)cc3Cl)ccc21 ; O=C1c2ccccc2C(Cc2nn(C3CC4(COC4)C3)cc2Cl)N1Cc1ccc2[nH]nnc2c1]

Step 1 was performed according to the procedure outlined for preparation of 372-7.

Step 2 was performed according to the procedure outlined for preparation of compound 7 to afford 412 as a white solid (34 mg, 54.8%). MS (EST) m/z 475 [M+H]⁺. ¹H NMR (400 MHz, DMSO-d₆) δ 8.85 (s, 1H), 7.92 (s, 1i), 7.82-7.78 (m, 2H), 7.57-7.47 (m, 2H), 7.15 (d, J=8.5 Hz, 1H), 7.06 (d, J=6.4 Hz, 1H), 5.41 (d, J=15.0 Hz, 1H), 5.11 (t, J=4.5 Hz, 1H), 4.77 (dd, J=9.5, 4.6 Hz, 1H), 4.58 (d, J=15.0 Hz, 1H), 4.52 (d, J=11.9 Hz, 1H), 4.33 (d, J=11.8 Hz, 1H), 3.89 (dd, J=14.7, 4.7 Hz, 1H), 3.39 (d, J=11.6 Hz, 1H), 3.31 (d, J=11.6 Hz, 1H), 3.04 (dd, J=14.7, 9.6 Hz, 1H), 2.56 (dd, J=9.9, 4.3 Hz, 1H), 2.43 (dd, J=10.0, 4.5 Hz, 1H), 1.94-1.85 (m, 2H). RT=1.96 min (HPLC)

### 2-((1H-benzo[d][1,2,3]triazol-5-yl)methyl)-3-((1-(azetidin-3-yl)-4-chloro-1H-pyrazol-3-yl)methyl)isoindolin-1-one (413)

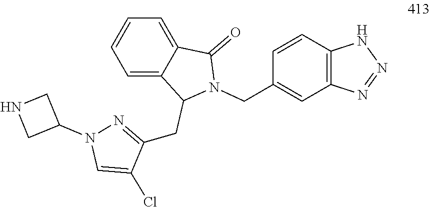 [SMILES: O=C1c2ccccc2C(Cc2nn(C3CNC3)cc2Cl)N1Cc1ccc2[nH]nnc2c1]

The title compound 413 was prepared according to the procedure described for compound 373 using tert-butyl 3-iodoazetidine-1-carboxylate as a starting material to afford a white solid (3 mg, 9.2%). MS (ESI) m/z 434 [M+H]⁺. RT=4.13 min (HPLC).

### 2-((1H-benzo[d][1,2,3]triazol-5-yl)methyl)-3-((4-chloro-1-methyl-1H-pyrazol-5-yl)methyl)-6-methoxyisoindolin-1-one (414)

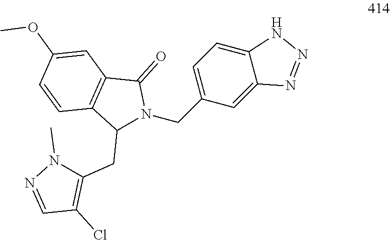 [SMILES: COc1ccc2c(c1)C(=O)N(Cc1ccc3[nH]nnc3c1)C2Cc1c(Cl)cnn1C]

The title compound 414 was prepared according to the procedure described for compound 279 as a white solid (20 mg, 22%). MS (ESI) m/z 423 [M+H]⁺. ¹H NMR (400 MHz, CDCl₃) δ 7.91 (d, J=8.5 Hz, 1H), 7.80 (s, 1H), 7.51 (s, 1H), 7.45 (d, J=2.4 Hz, 1H), 7.37 (d, J=8.5 Hz, 1H), 7.01 (dd, J=8.4, 2.4 Hz, 1H), 6.52 (d, J=8.4 Hz, 1H), 5.50 (d, J=15.2 Hz, 1H), 4.69 (dd, J=8.9, 6.0 Hz, 1H), 4.46 (d, J=15.2 Hz, 1H), 3.85 (s, 3H), 3.49 (s, 3H), 3.43 (dd, J=14.5, 6.0 Hz, 1H), 2.72 (dd, J=14.4, 9.0 Hz, 1H).

### 3-((3-bromopyridin-2-yl)methyl)-2-(4-methoxybenzyl)isoindolin-1-one (415)

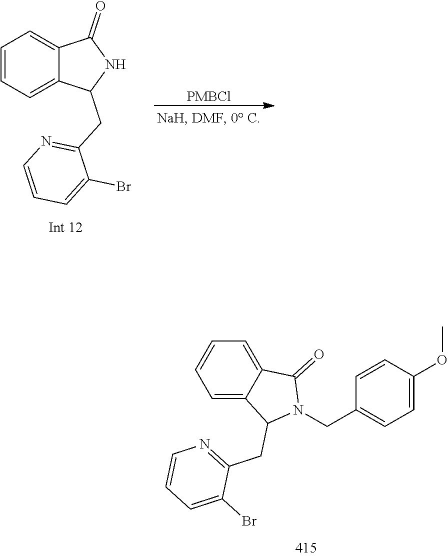 [SMILES: O=C1NC(Cc2ncccc2Br)c2ccccc21 ; "PMBCl" ; COc1ccc(CN2C(=O)c3ccccc3C2Cc2ncccc2Br)cc1]

To a solution of Int 12 (148 mg, 0.49 mmol) in DMF (2 mL) was added NaH (21 mg, 0.54 mmol) at 0° C. and the mixture was stirred for 1 h. Then 1-(chloromethyl)-4-methoxy-benzene (76 mg, 0.49 mmol) was added, and the mixture was stirred for another 2 h. The mixture was diluted with DCM, washed with water, dried over Na₂SO₄, filtered and concentrated under reduced pressure. The residue was purified by prep-HPLC to afford 415 (88 mg, 43%) as a white solid. MS (ESI) m/z 423 [M+H]⁺. ¹H NMR (400 MHz, DMSO-d₆) δ 8.56 (dd, J=4.6, 1.5 Hz, 1H), 8.06 (dd, J=8.1, 1.5 Hz, 1H), 7.79-7.69 (m, 1H), 7.56-7.42 (m, 2H), 7.30 (dd, J=8.1, 4.6 Hz, 1H), 7.06-7.00 (m, 2H), 7.00-6.93 (m, 1H), 6.89-6.75 (m, 2H), 5.13-4.97 (m, 2H), 4.30 (d, J=15.3 Hz, 1H), 3.71 (s, 3H), 3.60 (dd, J=14.9, 5.8 Hz, 1H), 3.17 (dd, J=14.9, 7.4 Hz, 1H).

### (E)-3-((3-bromopyridin-2-yl)methyl)-2-(2-methyl-3-(1H-1,2,3-triazol-4-yl)allyl)isoindolin-1-one (416)

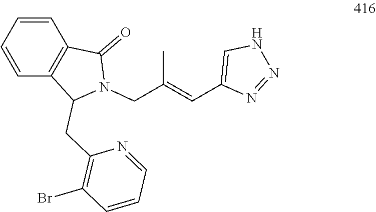 [SMILES: C/C(=C\c1c[nH]nn1)CN1C(=O)c2ccccc2C1Cc1ncccc1Br]

The title compound 416 was prepared according to the procedure described for compound 314 as a white solid (17 mg, 21.2%). MS (ESI) m/z 424 [M+H]⁺. ¹H NMR (400 MHz, DMSO-d₆) δ 8.56 (dd, J=4.8, 1.5 Hz, 1H), 8.06 (dd, J=8.1, 1.5 Hz, 1H), 7.83 (s, 1H), 7.77-7.70 (m, 1H), 7.54-7.48 (m, 2H), 7.30 (dd, J=8.1, 4.6 Hz, 1H), 7.09-7.02 (m, 11H), 6.08 (s, 1H), 5.24 (t, J=6.6 Hz, 1H), 4.58 (d, J=15.8 Hz, 1H), 3.96 (d, J=15.9 Hz, 1H), 3.64 (dd, J=14.9, 6.0 Hz, 11H), 5.23 (dd, J=14.8, 7.2 Hz, 1H), 1.81 (s, 3H).

### 2-(3-(2H-1,2,3-triazol-4-yl)prop-2-yn-1-yl)-3-((3-bromopyridin-2-yl)methyl) isoindolin-1-one (417)

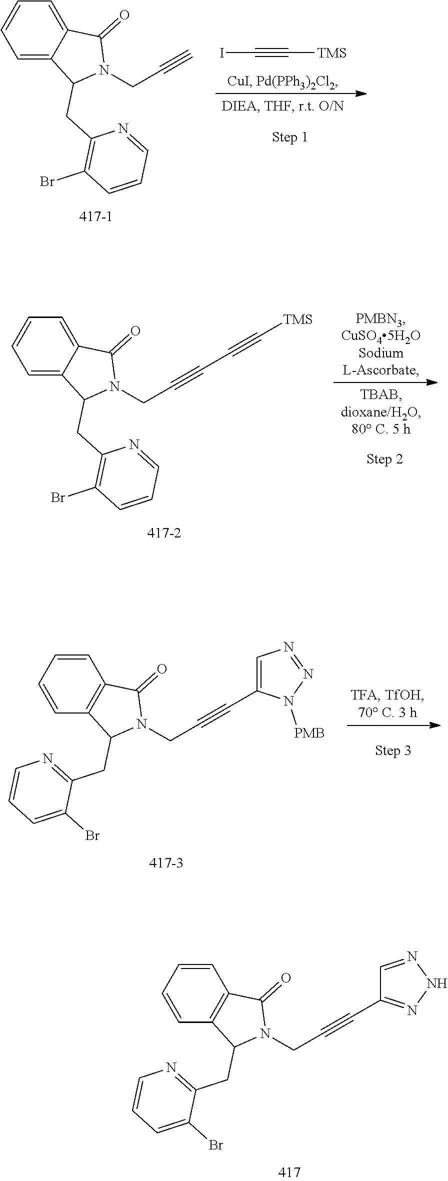

The intermediate 417-1 was prepared according to the procedure outlined for 222-2.

### Step 1: 3-((3-bromopyridin-2-yl)methyl)-2-(5-(trimethylsilyl)penta-2,4-diyn-1-yl)isoindolin-1-one (417-2)

To a solution of 417-1 (350 mg, 1.03 mmol), CuI (9.8 mg, 0.05 mmol), Pd(PPh₃)₂Cl₂ (36 mg, 0.05 mmol) and DIEA (398 mg, 3.08 mmol) in THF (25 mL) was added (iodoethynyl)trimethylsilane (277 mg, 1.23 mmol), the reaction mixture was stirred at r.t. overnight under N₂. The mixture was washed with water (25 mL*2) and dried over anhydrous Na₂SO₄, filtered and concentrated. The residue was purified by column chromatography on silica gel (PE/EA=2/1) to give 417-2 as a brown solid (200 mg, 44%). MS (ESI) m/z 437[M+H]⁺.

### Step 2: 3-((3-bromopyridin-2-yl)methyl)-2-(3-(1-(4-methoxybenzyl)-1H-1,2,3-triazol-5-yl)prop-2-yn-1-yl)isoindolin-1-one (417-3)

A mixture of 417-2 (40 mg, 0.09 mmol), CuSO₄-5H₂O (4.6 mg, 0.02 mmol), Sodium L-Ascorbate (4 mg, 0.02 mmol), TBAB (10 mg, 0.02 mmol) and PMBN₃ (18 mg, 0.11 mmol) in dioxane/H₂O (5 mL/1 mL) was stirred at 80° C. for 5 h under N₂. The mixture was extracted with EA (8 mL*2) and the combined organic layer was washed with water (10 mL*2) and dried over anhydrous Na₂SO₄, filtered and concentrated to give the crude product 417-3 as a brown oil (60 mg). MS (ESI) m/z 528 [M+H]⁺.

### Step 3: 2-(3-(2H-1,2,3-triazol-4-yl)prop-2-yn-1-yl)-3-((3-bromopyridin-2-yl)methyl)isoindolin-1-one (417)

A mixture of 417-3 (60 mg, 0.11 mmol), TfOH (51 mg, 0.34 mmol) in TFA (3 mL) was stirred at 70° C. for 3 h. The mixture was basified with Na₂CO₃ to pH=8 and extracted with EA (10 mL*2). The combined organic layer was washed with water (10 mL*2) and dried over anhydrous Na₂SO₄, filtered and concentrated. The residue was purified by prep-HPLC to give 417 as a white solid (6 mg, 16%, last 2 steps). MS (ESI) m/z 408 [M+H]⁺. ¹H NMR (400 MHz, DMSO-d₆): δ 8.60 (dd, J=4.8, 1.6 Hz, 1H), 8.08 (dd, J=7.6, 1.2 Hz, 2H), 7.72 (d, J=7.2 Hz, 1H), 7.56-7.48 (m, 2H), 7.30 (dd, J=8.0, 4.4 Hz, 1H), 7.14 (d, J=7.6 Hz, 1H), 5.46 (t, J=6.8 Hz, 1H), 4.90 (d, J=18.0 Hz, 1H), 4.47 (d, J=18.0 Hz, 1H), 3.75 (dd, J=15.1, 6.0 Hz, 1H), 3.31 (dd, J=14.8, 7.2 Hz, 1H).

### 2-((1H-benzo[d][1,2,3]triazol-5-yl)methyl)-3-((5-chlorothiazol-4-yl)methyl)isoindolin-1-one (418)

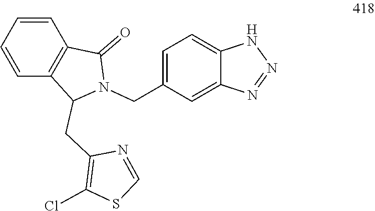 [SMILES: O=C1c2ccccc2C(Cc2ncsc2Cl)N1Cc1ccc2[nH]nnc2c1]

The title compound 418 was prepared according to the procedure described for compound 279 as a light yellow solid (35 mg, 35%). MS (ESI) m/z 396 [M+H]⁺. ¹H NMR (400 MHz, DMSO-d₆) δ 15.64 (s, 1H), 8.96 (s, 1H), 7.83-7.91 (m, 1H), 7.78-7.73 (m, 1H), 7.69 (dd, J=6.9, 1.2 Hz, 11H), 7.55-7.45 (m, 2H), 7.28 (d, J=8.5 Hz, 1H), 7.20 (d, J=7.4 Hz, 1H), 5.29 (d, J=15.5 Hz, 1H), 4.88 (dd, J=6.6, 4.7 Hz, 1H), 4.65 (d, J=15.5 Hz, 1H), 3.48 (dd, J=14.8, 4.8 Hz, 1H), 3.21 (dd, J=14.8, 6.6 Hz, 11H).

### 6-((6-bromo-1-((4-chloro-1-methyl-1H-pyrazol-5-yl)methyl)-1-methyl-3-oxoisoindolin-2-yl)methyl)benzo[d]oxazol-2(3H)-one (419)

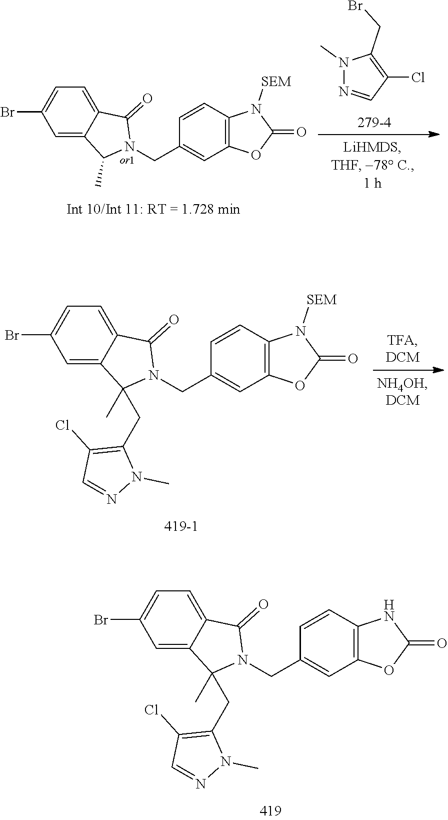

Step 1 was performed according to the procedure outlined for preparation of Int 12-2.

Step 2 was performed according to the procedure outlined for step 2 of general procedure E to afford 419 as a white solid (650 mg, 54.6%). MS (ESI) m/z 501 [M+H]⁺. ¹H NMR (400 MHz, DMSO-d₆) δ 11.59 (s, 1H), 7.65 (dd, J=8.0, 1.7 Hz, 1H), 7.61-7.53 (m, 2H), 7.38 (s, 1H), 7.30 (d, J=1.5 Hz, 1H), 7.17 (dd, J=8.1, 1.6 Hz, 1H), 7.02 (d, J=8.0 Hz, 1H), 5.08 (d, J=15.8 Hz, 1H), 4.67 (d, J=15.8 Hz, 1H), 3.44 (d, J=15.2 Hz, 1H), 3.42 (s, 3H), 3.25 (d, J=15.1 Hz, 1H), 1.46 (s, 3H).

### 2-((1H-benzo[d][1,2,3]triazol-5-yl)methyl)-3-((4-chloro-3-(dimethylamino)-1-methyl-1H-pyrazol-5-yl)methyl)isoindolin-1-one (420)

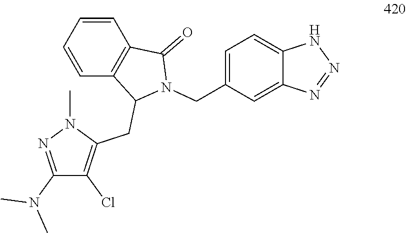 [SMILES: CN(C)c1nn(C)c(CC2c3ccccc3C(=O)N2Cc2ccc3[nH]nnc3c2)c1Cl]

The title compound 420 was prepared according to the procedure described for compound 279 as a white solid (17 mg, 27%). MS (ESI) m/z 436 [M+H]⁺. ¹H NMR (400 MHz, DMSO-d₆) δ 7.86 (d, J=8.5 Hz, 1H), 7.80-7.75 (m, 1H), 7.72 (s, 1H), 7.59-7.46 (m, 2H), 7.24 (dd, J=8.5, 1.5 Hz, 1H), 6.96-6.78 (m, 1H), 5.36 (d, J=15.3 Hz, 1H), 4.63 (dd, J=8.3, 6.1 Hz, 1H), 4.45 (d, J=15.3 Hz, 1H), 3.43 (dd, J=14.8, 6.0 Hz, 1H), 3.40 (s, 3H), 2.90 (d, J=14.7, 8.4 Hz, 1H), 2.74 (s, 6H).

The intermediate 420-3 was prepared as follows:

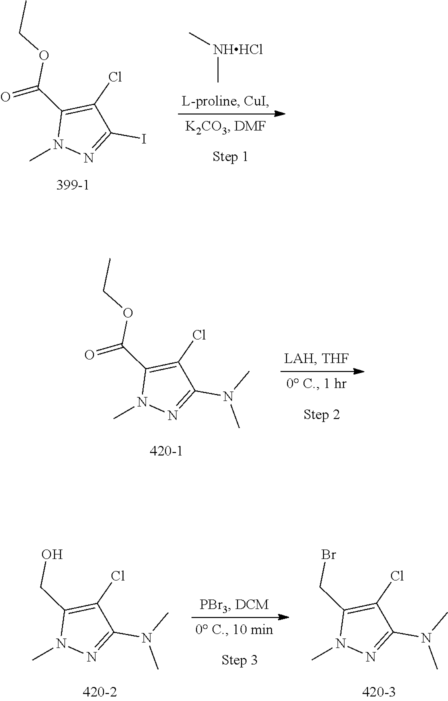

### Step 1. ethyl 4-chloro-3-(dimethylamino)-1-methyl-1H-pyrazole-5-carboxylate (420-1)

To a solution of 399-1 (1 g, 3.18 mmol) and dimethylamine hydrochloride (1.21 g, 15 mmol) in DMF (10 mL) were added L-proline (172 mg, 1.5 mmol), K₂CO₃ (880 mg, 6.36 mmol) and CuI (300 mg, 1.5 mmol). The resulting mixture was stirred for additional 12 h in a sealed tube at 90° C. under N₂. The mixture was allowed to cool down to r.t. The resulting mixture was diluted with water (20 mL) and extracted with EtOAc (3*20 mL). The combined organic layers were washed with brine (10 mL), dried over anhydrous Na₂SO₄, after filtration, the filtrate was concentrated under reduced pressure. The residue was purified by silica gel column chromatography eluting with (PE/EA=1/1) to afford 420-1 (180 mg, 24%) as a white solid. MS (ESI) m/z 232 [M+H]⁺.

Step 2 was performed according to the procedure outlined for preparation of 263-2.

Step 3 was performed according to the procedure outlined for preparation of 263-3.

### 2-((1H-benzo[d][1,2,3]triazol-5-yl)methyl)-3-((4-chloro-1-methyl-1H-pyrazol-5-yl)methyl)-5-(difluoromethoxy)isoindolin-1-one (421)

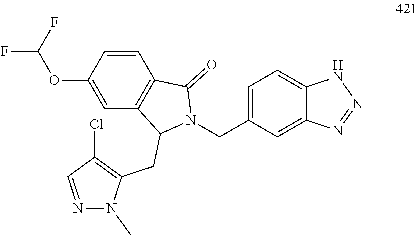 [SMILES: Cn1ncc(Cl)c1CC1c2cc(OC(F)F)ccc2C(=O)N1Cc1ccc2[nH]nnc2c1]

The title compound 421 was prepared according to the procedure described for compound 279 as a white solid (56 mg, 26%). MS (ESI) m/z 459 [M+H]⁺. ¹H NMR (400 MHz, CDCl₃) δ 7.94 (d, J=8.4 Hz, 1H), 7.90 (d, J=8.4 Hz, 1H), 7.77 (s, 1H), 7.51 (s, 1H), 7.37 (d, J=8.5 Hz, 1H), 7.29-7.22 (m, 1H), 6.49 (t, J=72.4 Hz, 1H), 6.41 (s, 1H), 5.50 (d, J=15.3 Hz, 1H), 4.72 (dd, J=8.8, 6.2 Hz, 1H), 4.41 (d, J=15.3 Hz, 1H), 3.51 (s, 3H), 3.42 (dd, J=14.6, 6.2 Hz, 1H), 2.78 (dd, J=14.6, 8.8 Hz, 1H).

The intermediate 421-2 was prepared as follows:

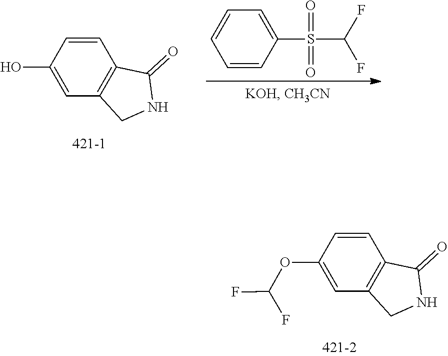

A mixture of 5-hydroxyisoindolin-1-one (650 mg, 4.4 mmol), ((difluoromethyl) sulfonyl)benzene (2.8 g, 15 mmol) and potassium hydroxide (560 mg, 10 mmol) in CH₃CN (10 mL) and water (2 mL) was stirred at 60′C for 2 days, then the mixture was diluted with water (10 mL) and extracted with ethyl acetate (2*25 mL). The combined organic layer was dried over anhydrous Na₂SO₄ and concentrated. The residue was purified by column chromatography on silica gel (eluting (PE/EA=2/1 to 1/1) to give 5-(difluoromethoxy)isoindolin-1-one (170 mg, 20%) as a white solid. MS (ESI) m/z 200 [M+H]⁺.

### 2-((1H-benzo[d][1,2,3]triazol-5-yl)methyl)-3-((4-chloro-1-methyl-1H-pyrazol-5-yl)methyl)-6-((methylamino)methyl)isoindolin-1-one (422)

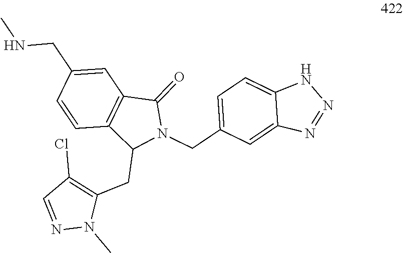 [SMILES: CNCc1ccc2c(c1)C(=O)N(Cc1ccc3[nH]nnc3c1)C2Cc1c(Cl)cnn1C]

The title compound 422 was prepared according to the procedure described for compound 279 as a white solid (50 mg, 42%). MS (ESI) m/z 436 [M+H]⁺. ¹H NMR (400 MHz, CDCl₃) δ 8.01 (s, 1H), 7.86 (d, J=8.2 Hz, 1H), 7.57 (s, 1H), 7.52-7.46 (m, 2H), 7.23 (s, 1H), 6.68 (d, J=7.8 Hz, 1H), 5.46 (d, J=15.6 Hz, 1H), 4.70 (dd, J=8.4, 6.4 Hz, 1H), 4.36 (d, J=15.3 Hz, 1H), 4.05 (d, J=8.9 Hz, 2H), 3.47 (s, 3H), 3.34 (dd, J=14.6, 6.3 Hz, 1H), 2.77 (dd, J=14.6, 8.6 Hz, 1H), 2.60 (s, 3H).

The intermediate 422-5 was prepared as follows:

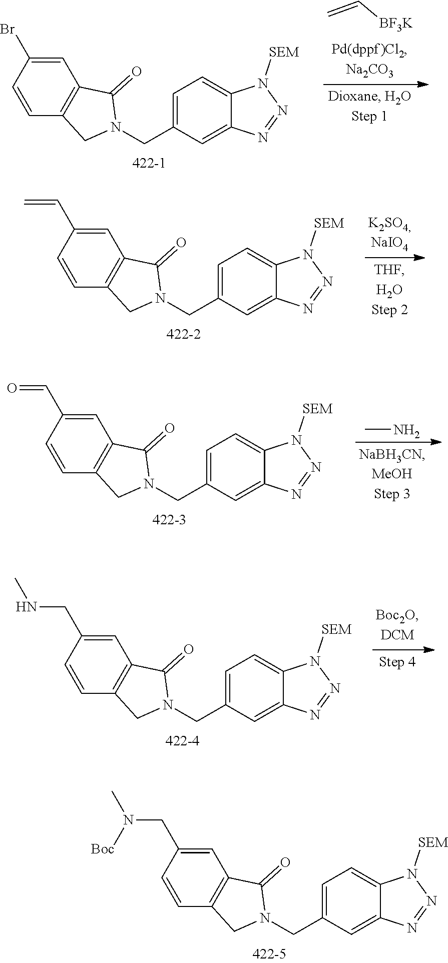

Step 1 was performed according to the procedure outlined for preparation of 26-1.

Step 2 was performed according to the procedure outlined for preparation of 381-6.

Step 3 was performed according to the procedure outlined for preparation of 273-2.

Step 4 was performed according to the procedure outlined for preparation of Int 12-1.

### (E)-2-(3-(1H-pyrazol-4-yl)allyl)-3-((3-bromopyridin-2-yl)methyl)isoindolin-1-one (423)

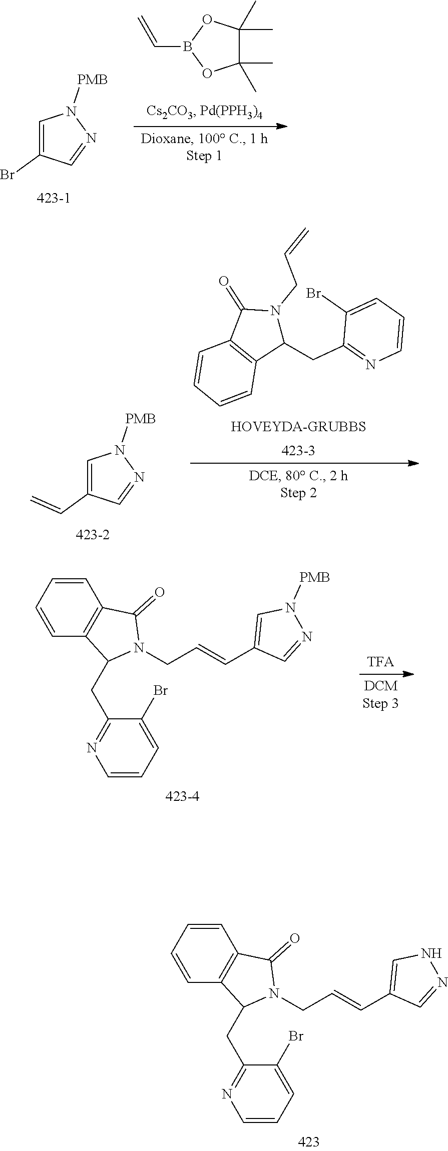

Step 1 was performed according to the procedure outlined for preparation of 26-1.

Step 2 was performed according to the procedure outlined for preparation of 312-4.

Step 3 was performed according to the procedure outlined for preparation of 312-5 to afford compound 423 as a white solid (5 mg, 12%). MS (ESI) m/z 409 [M+H]⁺. ¹H NMR (400 MHz, CD₃OD) δ 8.44 (d, J=4.7, 1.5 Hz, 1H), 7.92 (dd, J=8.1, 1.5 Hz, 1H), 7.71-7.58 (m, 3H), 7.45-7.35 (m, 2H), 7.17 (dd, J=8.1, 4.7 Hz, 1H), 6.98-6.93 (m, 1H), 6.16 (d, J=16.1 Hz, 1H), 5.86 (ddd, J=15.9, 7.5, 5.3 Hz, 1H), 5.30 (t, J=6.7 Hz, 1H), 4.59 (ddd, J=15.6, 5.4, 1.6 Hz, 1H), 3.89 (dd, J=15.7, 7.4 Hz, 1H), 3.61 (dd, J=14.6, 6.3 Hz, 1H), 3.24-3.18 (m, 1H).

### 3-((3-bromopyridin-2-yl)methyl)-2-((7-oxo-6-azaspiro[3.4]octan-2-yl)methyl) isoindolin-1-one (424)

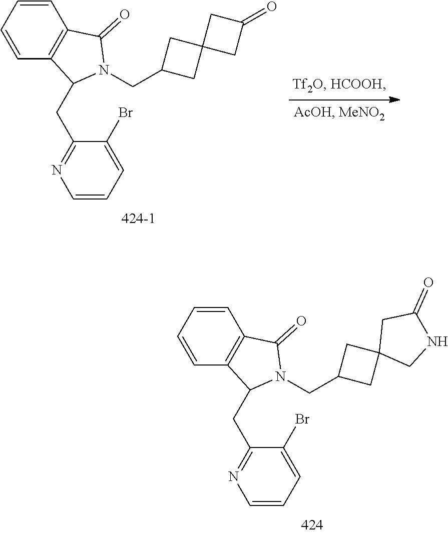 [SMILES: O=C1CC2(C1)CC(CN1C(=O)c3ccccc3C1Cc1ncccc1Br)C2 ; O=C1CC2(CN1)CC(CN1C(=O)c3ccccc3C1Cc1ncccc1Br)C2]

The intermediate 424-1 was prepared according to the procedure outlined for 270-3. A mixture of 424-1 (200 mg, 0.47 mmol), Tf₂O (265 mg, 0.94 mmol), HCOOH (54 mg, 1.18 mmol) in AcOH/MeNO₂ (3 mL/3 mL) was stirred at 100° C. for 4 h. Water (10 mL) was added, the mixture was extracted with EA (20 mL*3), and the combined organic layers were washed with sat. NaHCO₃ (aq.) until pH 8. The organic layer was concentrated under vacuum. The residue was purified by prep-HPLC to give 424 as a white solid (22 mg, 11%). MS (ESI) m/z 440 [M+H]⁺. ¹H NMR (400 MHz, CD₃OD) δ 8.56 (d, J=4.0 Hz, 1H), 8.04 (dd, J=8.0, 1.2 Hz, 1H), 7.75-7.73 (m, 1H), 7.49-7.46 (m, 2H), 7.26 (dd, J=8.0, 4.4 Hz, 1H), 6.99-6.96 (m, 1H), 5.31 (t, J=6.8 Hz, 1H), 4.07-4.00 (m, 1H), 3.69 (dd, J=14.4, 5.6 Hz, 1H), 3.42 (s, 1H), 3.35-3.31 (m, 2H), 3.30-3.21 (m, 2H), 2.66-2.60 (m, 1H), 2.43 (s, 1H), 2.34 (s, 1H), 2.20-2.03 (m, 2H), 1.95-1.81 (m, 2H).

### (E)-2-(3-(1H-1,2,3-triazol-4-yl)but-2-en-1-yl)-3-((3-bromopyridin-2-yl)methyl) isoindolin-1-one (425)

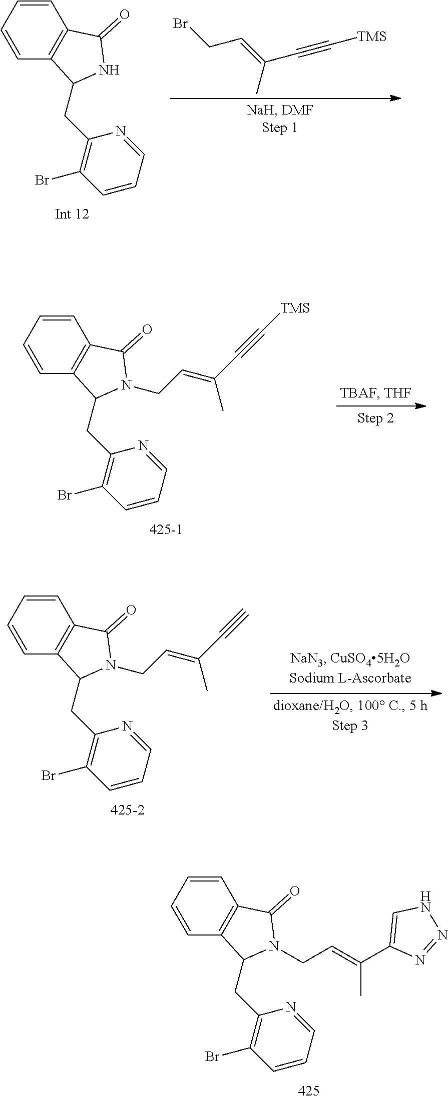 [SMILES: O=C1NC(Cc2ncccc2Br)c2ccccc21 ; C/C(C#C[Si](C)(C)C)=C\CBr ; C/C(C#C[Si](C)(C)C)=C\CN1C(=O)c2ccccc2C1Cc1ncccc1Br ; C#C/C(C)=C/CN1C(=O)c2ccccc2C1Cc1ncccc1Br ; C/C(=C\CN1C(=O)c2ccccc2C1Cc1ncccc1Br)c1c[nH]nn1]

Step 1 was performed according to the procedure outlined for preparation of 222-1.

### Step 2. (E)-3-((3-bromopyridin-2-yl)methyl)-2-(3-methylpent-2-en-4-yn-1-yl)isoindolin-1-one (425-2)

To a solution of 425-1 in THF (20 mL) was added TBAF (5.0 mL, 1.0 M in THF) at 0° C. The mixture was stirred at r.t. for 2 h. The mixture was added water (20 mL), extracted with EA (20 mL*3), washed with brine (20 mL), dried over anhydrous Na₂SO₄, filtered and concentrated to give 425-2 (830 mg, 90%) as a yellow oil. MS (ESI) m/z 381 [M+H]⁺.

Step 3 was performed according to the procedure described for step 6 for preparation of 403 to afford 425 (5 mg, 0.6%) as a yellow solid. MS (ESI) m/z 424 [M+H]⁺. ¹H NMR (400 MHz, CD₃OD): S 8.52 (d, J=4.0 Hz, 1H), 8.00 (dd, J=8.0, 1.2 Hz, 1H), 7.88 (s, 1H), 7.78-7.77 (m, 1H), 7.51-7.48 (m, 2H), 7.24 (dd, J=8.4, 4.8 Hz, 1H), 7.06-7.04 (m, 1H), 6.14 (t, J=6.4 Hz, 1H), 5.41 (t, J=6.4 Hz, 1H), 4.73 (dd, J=16.0, 6.0 Hz, 1H), 4.24 (dd, J=16.4, 7.2 Hz, 1H), 3.72 (dd, J=14.8, 6.0 Hz, 1H), 3.31-3.29 (m, 1H), 2.06 (s, 3H).

### (2s,4S)-2-(((R)-1-((4-chloro-1-methyl-1H-pyrazol-5-yl)methyl)-4-fluoro-3-oxoisoindolin-2-yl)methyl)-5-oxa-7-azaspiro[3.4]octan-6-one (426) and (2s,4R)-2-(((S)-1-((4-chloro-1-methyl-1H-pyrazol-5-yl)methyl)-4-fluoro-3-oxoisoindolin-2-yl)methyl)-5-oxa-7-azaspiro[3.4]octan-6-one (427)

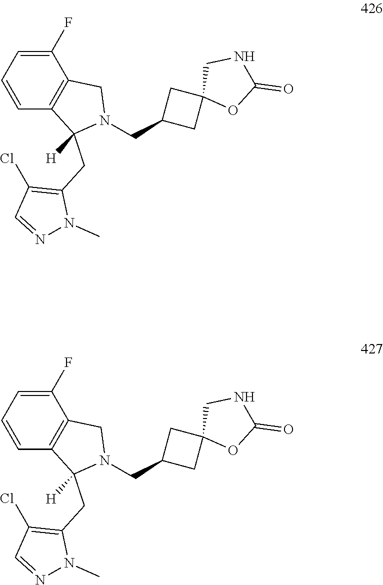 [SMILES: Cn1ncc(Cl)c1C[C@@H]1c2cccc(F)c2CN1C[C@H]1C[C@]2(CNC(=O)O2)C1 ; Cn1ncc(Cl)c1C[C@H]1c2cccc(F)c2CN1C[C@H]1C[C@]2(CNC(=O)O2)C1]

Chiral separation of compound 309 by chiral prep-HPLC (column: CHIRALPAK IA; column size: 2 cm×25 cm, 5 μm; mobile phase A: HEX; mobile phase B: EtOH) was conducted to afford two enantiomers: compound 426 and compound 427. One of the two enantiomers was a white solid, with RT=2.312 min (CHIRAL HPLC, column: CHIRALPAK IA-3; column size: 4.6*50 mm, 3 μm; mobile phase: Hex(0.1% DEA):EtOH=70:30; flow: 1.0 mL/min). Mass (m/z): 419 [M+H]⁺. ¹H NMR (400 MHz, DMSO-d₆) δ 7.59-7.47 (m, 3H), 7.25 (t, J=9.8 Hz, 1H), 6.61 (d, J=7.5 Hz, 1H), 4.90 (dd, J=8.5, 5.6 Hz, 1H), 3.95 (dd, J=14.2, 6.0 Hz, 1H), 3.65 (s, 3H), 3.56 (dd, J=14.9, 5.7 Hz, 1H), 3.50 (s, 2H), 3.30 (dd, J=14.2, 6.4 Hz, 1H), 2.92 (dd, J=14.8, 8.6 Hz, 1H), 2.41-2.04 (m, 5H). The other one of the two enantiomers was a white solid, with RT=3.262 min (CHIRAL HPLC, column: CHIRALPAK IA-3; column size: 4.6*50 mm, 3 μm; mobile phase: Hex(0.1% DEA):EtOH=70:30; flow: 1.0 mL/min). Mass (m/z): 419 [M+H]⁺. ¹H NMR (400 MHz, DMSO-d(,) δ 7.59-7.47 (m, 3H), 7.25 (t, J=9.8 Hz, 1H), 6.61 (d, J=7.5 Hz, 1H), 4.90 (dd, J=8.5, 5.6 Hz, 1H), 3.95 (dd, J=14.2, 6.0 Hz, 1H), 3.65 (s, 3H), 3.56 (dd, J=14.9, 5.7 Hz, 1H), 3.50 (s, 2H), 3.31-3.25 (m, 1H), 2.92 (dd, J=14.8, 8.6 Hz, 1H), 2.41-2.04 (m, 5H).

### 3-((4-fluoro-3-oxo-2-((6-oxo-5-oxa-7-azaspiro[3.4]octan-2-yl)methyl)isoindolin-1-yl)methyl)-4-methylpicolinonitrile (428)

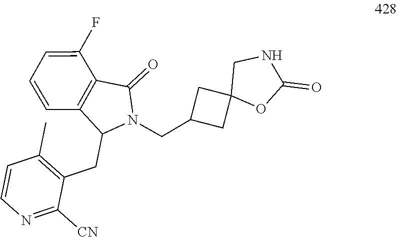 [SMILES: Cc1ccnc(C#N)c1CC1c2cccc(F)c2C(=O)N1CC1CC2(CNC(=O)O2)C1]

The title compound 428 was prepared according to the procedure described for compound 308 (80 mg, 59.7%) as a white solid. MS (ESI) m/z 421 [M+H]⁺. ¹H NMR (400 MHz, CDCl₃) δ 8.58 (d, J=4.9 Hz, 1H), 7.37 (d, J=4.9 Hz, 1H), 7.31 (td, J=8.0, 4.7 Hz, 1H), 7.11 (t, J=8.6 Hz, 1H), 6.18 (d, J=7.5 Hz, 1H), 4.96 (s, 1H), 4.92 (dd, J=10.0, 5.7 Hz, 1H), 4.20 (dd, J=14.2, 7.7 Hz, 1H), 3.69 (dd, J=13.8, 5.7 Hz, 1H), 3.62 (s, 2H), 3.31 (dd, J=14.3, 5.9 Hz, 1H), 2.79 (dd, J=13.7, 10.0 Hz, 1H), 2.57-2.33 (m, 5H), 2.02 (s, 3H).

### 3-((3-bromopyridin-2-yl)methyl)-2-(4-hydroxybenzyl)isoindolin-1-one (429)

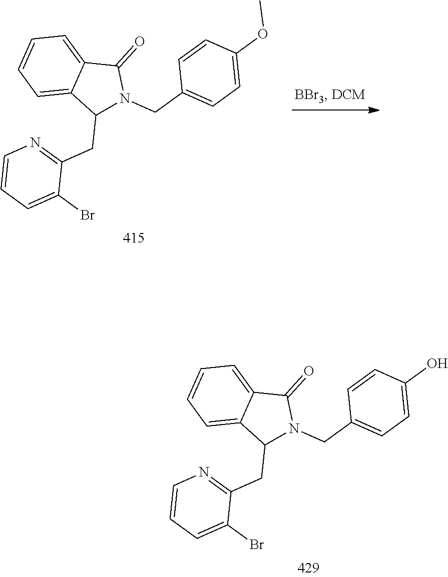 [SMILES: COc1ccc(CN2C(=O)c3ccccc3C2Cc2ncccc2Br)cc1 ; O=C1c2ccccc2C(Cc2ncccc2Br)N1Cc1ccc(O)cc1]

To a solution of 415 (20 mg, 0.047 mmol) in DCM (5 mL) was added BBr₃ (118 mg, 0.47 mmol) at −78° C. and the mixture was stirred for 1 h at 0° C. The reaction was monitored by LC/MS. Once completed, the mixture was diluted with DCM, washed with water, dried over Na₂SO₄, filtered and concentrated under reduced pressure. The residue was purified by prep-HPLC to give 429 (8 mg, 41.6%) as a white solid. MS (ESI) m/z 409 [M+H]⁺. ¹H NMR (400 MHz, DMSO-d₆) δ 8.56 (dd, J=4.6, 1.5 Hz, 1H), 8.07 (dd, J=8.1, 1.5 Hz, 1H), 7.78-7.66 (m, 1H), 7.53-7.42 (m, 2H), 7.30 (dd, J=8.1, 4.6 Hz, 1H), 7.01-6.92 (m, 1H), 6.92-6.84 (m, 2H), 6.70-6.58 (m, 2H), 5.10-4.95 (m, 2H), 4.22 (d, J=15.2 Hz, 1H), 3.60 (dd, J=14.8, 5.8 Hz, 1H), 3.16 (dd, J=14.8, 7.5 Hz, 1H).

### 6-((4-chloro-1-methyl-1H-pyrazol-5-yl)methyl)-5-(4-hydroxybenzyl)-5,6-dihydro-4H-pyrrolo[3,4-d]thiazol-4-one (430)

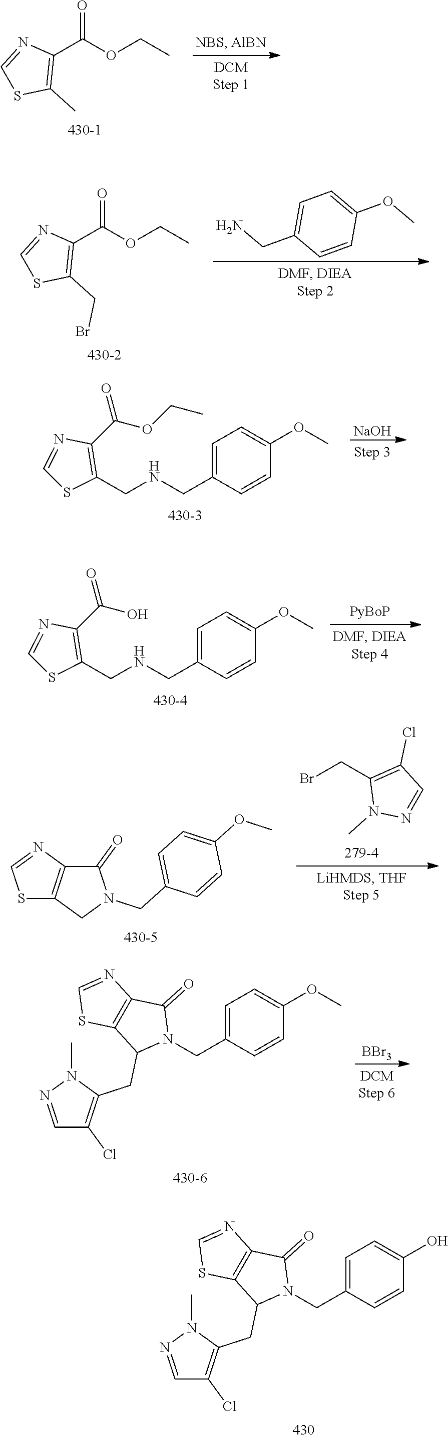

Step 1 was performed according to the procedure outlined for preparation of 222-1.

### Step 2. ethyl 5-(((4-methoxybenzyl)amino)methyl)thiazole-4-carboxylate (430-3)

To a solution of (4-methoxyphenyl)methanamine (724 mg, 5.28 mmol) and DIEA (2.3 mL, 13.2 mmol) in DMF (10 mL) was added ethyl 5-(bromomethyl)thiazole-4-carboxylate (1.1 g, 4.40 mmol) under N₂. The mixture was stirred for 2 h. The solvent was removed in vacuo and the residue was purified by C18 column to give 430-3 (940 mg, 69%) as a brown solid. MS (ESI) m/z 307 [M+H]⁺.

### Step 3. 5-(((4-methoxybenzyl)amino)methyl)thiazole-4-carboxylic acid (430-4)

To a solution of 430-3 (940 mg, 3.07 mmol) in THF (9 mL) and water (3 mL) was added NaOH (184 mg, 4.60 mmol) under N₂. The mixture was stirred for 2 h. The mixture was concentrated to give 430-4 (900 mg) as a white solid, which was used in the next step without further purification. MS (ESI) m/z 279 [M+H]⁺.

### Step 4. 5-(4-methoxybenzyl)-5,6-dihydro-4H-pyrrolo[3,4-d]thiazol-4-one (430-5)

To a solution of 430-4 (900 mg) in DMF (20 mL) were added DIEA (1.0 mL, 5.99 mmol) and PyBOP (2.34 g, 4.50 mmol) under N₂. The mixture was stirred for 1 h, water was added at 0′C and then extracted with EtOAc. The organic layer was separated, dried over anhydrous Na₂SO₄ and concentrated under vacuum. The residue was purified by silica gel column chromatography (PE/EA=1/5) to give 430-5 (370 mg, 47%) as a white solid. MS (ESI) m/z 261 [M+H]⁺.

Step 5 was performed according to the procedure outlined for preparation of Int 12-2.

Step 6 was performed according to the procedure outlined for step 2 for preparation of compound 14 to afford 430 as a white solid (38 mg, 54%). MS (ESI) m/z 375 [M+H]⁺. ¹H NMR (400 MHz, DMSO-d₆) δ 9.41 (s, 1H), 9.18 (s, 1H), 7.57 (s, 1H), 7.11 (d, J=8.0 Hz, 2H), 6.73 (d, J=8.0 Hz, 2H), 5.05 (d, J=15.1 Hz, 1H), 4.80 (dd, J=9.4, 5.8 Hz, 1H), 4.23 (d, J=15.1 Hz, 1H), 3.66 (s, 3H), 3.59 (dd, J=14.6, 5.9 Hz, 1H), 2.89 (dd, J=14.6, 9.5 Hz, 1H).

### 6-((5-bromo-3-((4-chloro-1-methyl-1H-pyrazol-5-yl)methyl)-1-oxoisoindolin-2-yl)methyl)benzo[d]oxazol-2(3H)-one (431)

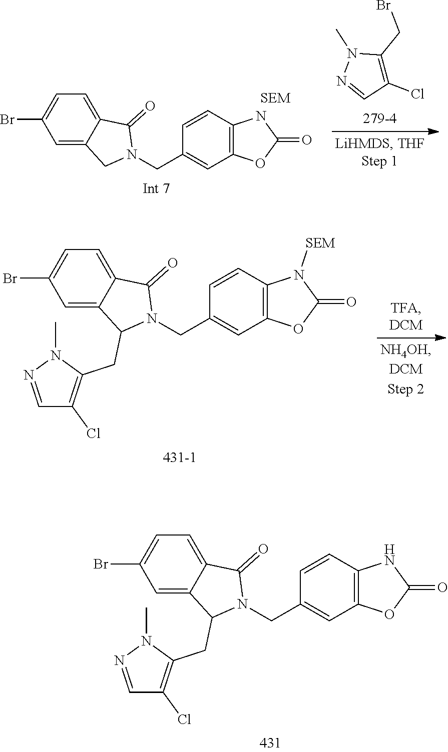

Step 1 was performed according to the procedure outlined for preparation of Int 12-2.

Step 2 was performed according to the procedure outlined for step 2 of general procedure E to afford 431 (21 mg, 16%) as a white solid. MS (ESI) m/z 487 [M+H]⁺. ¹H NMR (400 MHz, DMSO-d₆) δ 11.62 (s, 1H), 7.74-7.62 (m, 2H), 7.55 (s, 1H), 7.17 (d, J=1.6 Hz, 1H), 7.10 (d, J=1.5 Hz, 1H), 7.04 (d, J=7.9 Hz, 1H), 6.97 (dd, J=8.0, 1.6 Hz, 1H), 5.19 (d, J=15.2 Hz, 1H), 4.67 (t, J=6.8 Hz, 1H), 4.28 (d, J=15.2 Hz, 1H), 3.61 (s, 3H), 3.47 (dd, J=15.0, 6.1 Hz, 1H), 3.07 (dd, J=14.9, 7.6 Hz, 1H).

### 2-(1-((4-chloro-1-methyl-1H-pyrazol-5-yl)methyl)-3-oxo-2-((6-oxo-5-oxa-7-azaspiro[3.4]octan-2-yl)methyl)isoindolin-4-yl)acetonitrile (432)

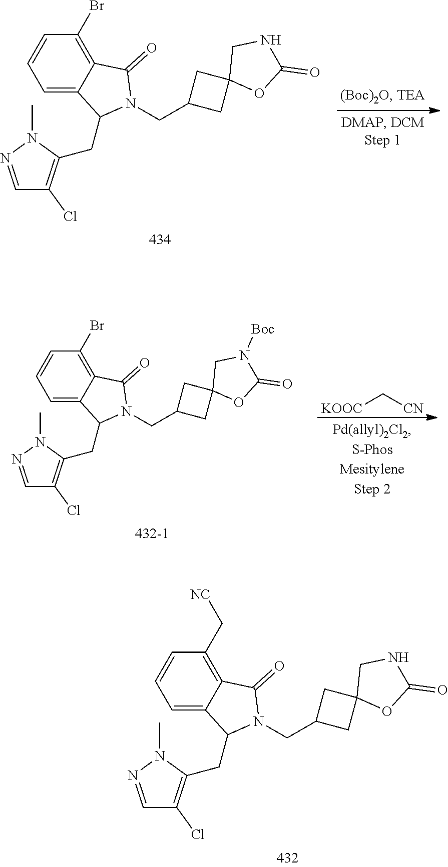

Step 1 was performed according to the procedure outlined for preparation of Int 12-1.

### Step 2. 2-(1-((4-chloro-1-methyl-1H-pyrazol-5-yl)methyl)-3-oxo-2-((6-oxo-5-oxa-7-azaspiro[3.4]octan-2-yl)methyl)isoindolin-4-yl)acetonitrile (432)

To a solution of tert-butyl 2-((4-bromo-1-((4-chloro-1-methyl-1H-pyrazol-5-yl)methyl)-3-oxoisoindolin-2-yl)methyl)-6-oxo-5-oxa-7-azaspiro[3.4]octane-7-carboxylate (150 mg, 0.26 mmol) in Mesitylene (3 mL) were added Pd(allyl)₂Cl₂ (5 mg, 0.013 mmol), S-Phos (11 mg, 0.026 mmol) and potassium 2-cyanoacetate (38 mg, 0.31 mmol) under N₂. The resulting mixture was stirred at 140° C. for 7 h. The reaction mixture was diluted with water and extracted with ethyl acetate. The combined organic phase was dried and concentrated. The residue was purified by Prep-HPLC to afford 432 (26 mg, 23%) as a white solid. MS (ESI) m/z 440 [M+H]⁺. ¹H NMR (400 MHz, CDCl₃) δ 7.57 (d, J=7.7 Hz, 1H), 7.53 (s, 1H), 7.45 (t, J=7.7 Hz, 1H), 6.52 (d, J=7.6 Hz, 1H), 5.23 (s, 1H), 4.84 (dd, J=9.8, 5.5 Hz, 1H), 4.43 (d, J=6.8 Hz, 2H), 4.17 (dd, J=14.4, 5.4 Hz, 1H), 3.61 (s, 2H), 3.53-3.44 (m, 4H), 3.19 (dd, J=14.4, 6.6 Hz, 1H), 2.59 (dd, J=14.4, 9.8 Hz, 1H), 2.51-2.26 (m, 5H).

### 2-((1-((4-chloro-1-methyl-1H-pyrazol-5-yl)methyl)-4-methoxy-3-oxoisoindolin-2-yl)methyl)-5-oxa-7-azaspiro[3.4]octan-6-one (433)

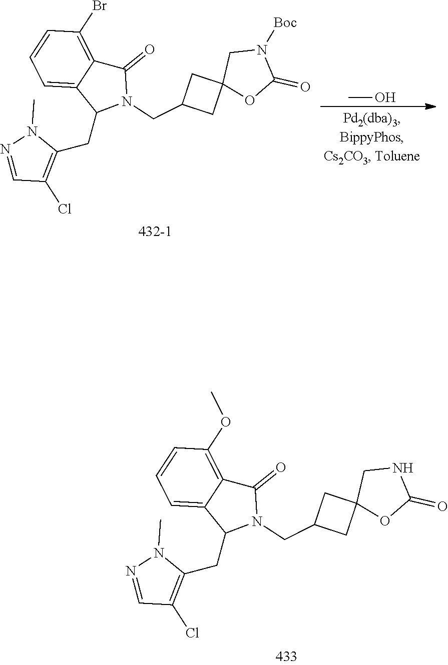

To a solution of 432-1(120 mg, 0.21 mmol) and methanol (13 mg, 0.41 mmol) in toluene (3 mL) were added Pd₂(dba)₃ (9 mg, 0.01 mmol), BippyPhos (11 mg, 0.02 mmol) and Cs₂CO₃ (135 mg, 0.41 mmol) under N₂. The resulting mixture was stirred at 100° C. overnight. The reaction mixture was diluted with water and extracted with ethyl acetate. The combined organic phase was dried and concentrated. The residue was purified by Prep-HPLC to afford 433 (7 mg, 7.9%) as a white solid. MS (ESI) m/z 431 [M+H]⁺. ¹H NMR (400 MHz, CDCl₃) δ 7.51 (s, 1H), 7.36 (t, J=7.9 Hz, 1H), 6.90 (d, J=8.3 Hz, 1H), 6.12 (d, J=7.6 Hz, 1H), 4.93 (s, 1H), 4.77 (dd, J=9.5, 5.6 Hz, 1H), 4.19-4.10 (m, 1H), 3.96 (s, 3H), 3.59 (s, 2H), 3.47 (s, 3H), 3.42 (dd, J=14.4, 5.7 Hz, 1H), 3.15-3.05 (m, 1H), 2.59 (dd, J=14.4, 9.5 Hz, 1H), 2.43-2.31 (m, 5H).

### 2-((4-bromo-1-((4-chloro-1-methyl-1H-pyrazol-5-yl)methyl)-3-oxoisoindolin-2-yl)methyl)-5-oxa-7-azaspiro[3.4]octan-6-one (434)

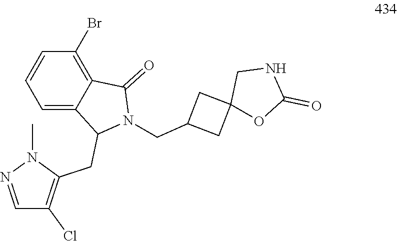 [SMILES: Cn1ncc(Cl)c1CC1c2cccc(Br)c2C(=O)N1CC1CC2(CNC(=O)O2)C1]

The title compound 434 was prepared according to the procedure described for compound 307 as a white solid (19 mg, 28.7%). MS (ESI) m/z 479 [M+H]⁺. ¹H NMR (400 MHz, CDCl₃) δ 7.62 (d, J=7.9 Hz, 1H), 7.52 (s, 1H), 7.25 (t, J=7.5 Hz, 1H), 6.48 (d, J=7.6 Hz, 1H), 5.09 (s, 1H), 4.79 (dd, J=9.7, 5.6 Hz, 1H), 4.17 (dd, J=14.8, 2.8 Hz, 1H), 3.61 (s, 2H), 3.52-3.43 (m, 4H), 3.22-3.12 (m, 1H), 2.59 (dd, J=14.4, 9.7 Hz, 1H), 2.47-2.32 (m, 5H).

### 2-((1H-benzo[d][1,2,3]triazol-5-yl)methyl)-3-((4-chloro-1-methyl-1H-pyrazol-5-yl)methyl)-5-(difluoromethyl)isoindolin-1-one (435)

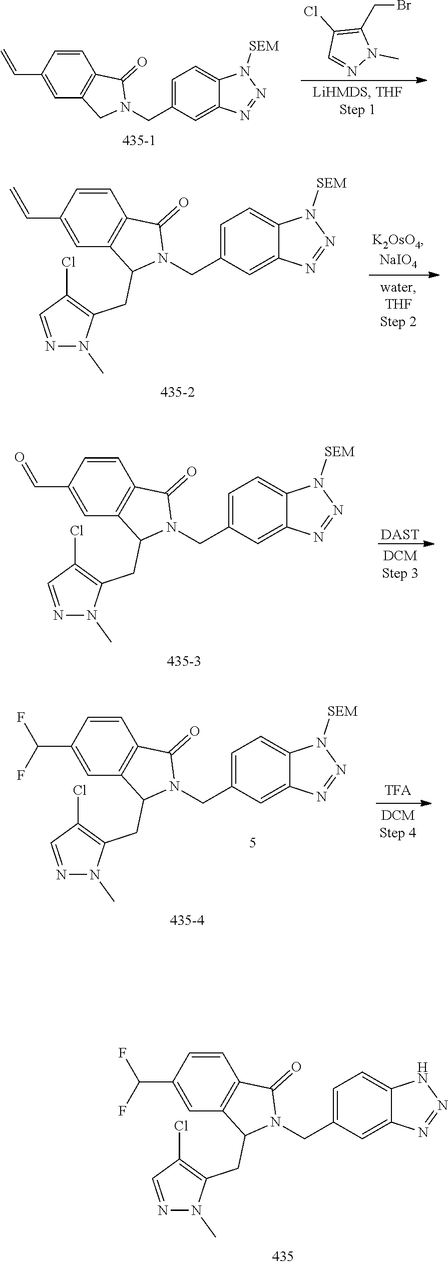

435-1 was prepared according to the procedure described for 422-2 as a yellow oil (2.5 g, 56%). MS (ESI) m/z 421 [M+H]⁺.

Step 1 was performed according to the procedure outlined for preparation of Int 12-2.

Step 2 was performed according to the procedure outlined for preparation of 381-6.

### Step 3. 3-((4-chloro-1-methyl-1H-pyrazol-5-yl)methyl)-5-(difluoromethyl)-2-((1-((2-(trimethylsilyl)ethoxy)methyl)-1H-benzo[d][1,2,3]triazol-5-yl)methyl)isoindolin-1-one (435-4)

To a solution of 435-3 (350 mg, 0.63 mmol) in DCM (6 mL) was added DAST (225 mg, 1.4 mmol) at 0° C., and stirred at 25 T under N₂ for 3 h. The mixture was diluted with ethyl acetate (20 mL), washed with water (20 mL) and brine (20 mL), dried over Na₂SO₄, filtered, and concentrated in vacuo. The residue was purified by column chromatography on silica gel (eluting with PE/EA=1/1 to 1/2) to give 435-4 (220 mg, 61%) as a yellow oil. MS (ESI) m/z 573 [M+H]⁺.

Step 4 was performed according to the procedure outlined for step 2 for preparation of compound 7 to give 435 as a white solid (57 mg, 37%). MS (ESI) m/z 443 [M+H]⁺. ¹H NMR (400 MHz, CDCl₃) δ 8.04 (d, J=8.4 Hz, 1H), 7.88 (d, J=8.5 Hz, 1H), 7.71 (s, 1H), 7.66 (d, J=7.9 Hz, 1H), 7.52 (s, 1H), 7.34 (dd, J=8.6, 1.2 Hz, 1H), 6.87 (s, 1H), 6.62 (t, J=56 Hz, 1H), 5.54 (d, J=15.2 Hz, 1H), 4.75 (dd, J=8.4, 6.4 Hz, 1H), 4.38 (d, J=15.3 Hz, 1H), 3.49 (s, 3H), 3.40 (dd, J=14.7, 6.5 Hz, 1H), 2.81 (dd, J=14.7, 8.5 Hz, 1H).

### 2-((1H-benzo[d][1,2,3]triazol-5-yl)methyl)-3-((4-chloro-1-methyl-1H-pyrazol-5-yl)methyl)-5-(methoxymethyl)isoindolin-1-one (436)

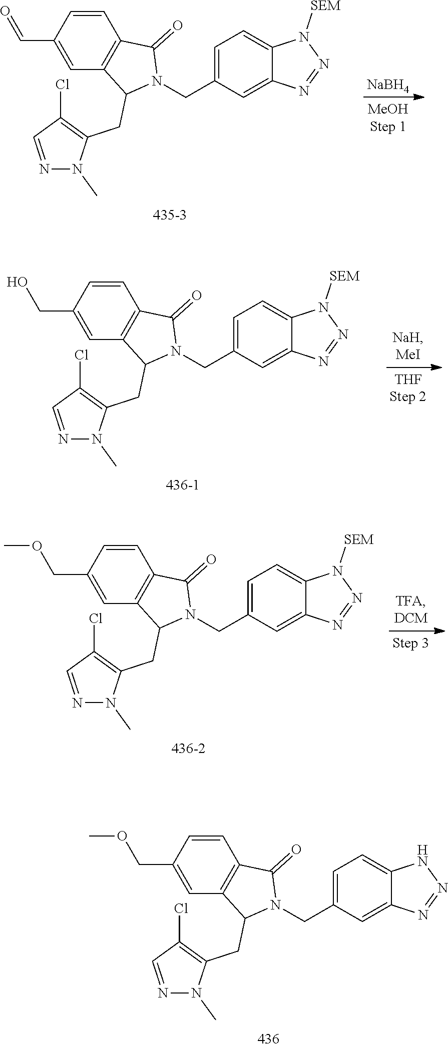 [SMILES: Cn1ncc(Cl)c1CC1c2cc(C=O)ccc2C(=O)N1Cc1ccc2c(c1)nnn2COCC[Si](C)(C)C ; "NaBH4" ; Cn1ncc(Cl)c1CC1c2cc(CO)ccc2C(=O)N1Cc1ccc2c(c1)nnn2COCC[Si](C)(C)C ; COCc1ccc2c(c1)C(Cc1c(Cl)cnn1C)N(Cc1ccc3c(c1)nnn3COCC[Si](C)(C)C)C2=O ; COCc1ccc2c(c1)C(Cc1c(Cl)cnn1C)N(Cc1ccc3[nH]nnc3c1)C2=O]

Step 1 was performed according to the procedure outlined for preparation of 164-2.

Step 2 was performed according to the procedure outlined for preparation of 46-3.

Step 3 was performed according to the procedure outlined for preparation of compound 7 to give 436 as a white solid (30 mg, 37%). MS (ESI) m/z 437 [M+H]⁺. ¹H NMR (400 MHz, CDCl₃) δ 7.91 (d, J=7.6 Hz, 1H), 7.86 (d, J=8.0 Hz, 11H), 7.65 (s, 1H), 7.51 (s, 1H), 7.46 (d, J=7.6 Hz, 1H), 7.28 (d, J=8.3 Hz, 1H), 6.76 (s, 1H), 5.52 (d, J=15.0 Hz, 1H), 4.70 (t, J=7.2 Hz, 1H), 4.43 (s, 2H), 4.33 (d, J=15.1 Hz, 1H), 3.49 (s, 3H), 3.38 (s, 3H), 3.36-3.00 (m, 1H), 2.83 (dd, J=13.4, 6.6 Hz, 1H).

### 2-((1H-benzo[d][1,2,3]triazol-5-yl)methyl)-5-(azetidin-3-yloxy)-3-((4-chloro-1-methyl-1H-pyrazol-5-yl)methyl)isoindolin-1-one (437)

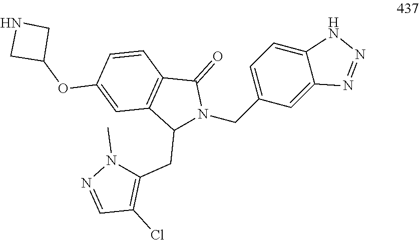 [SMILES: Cn1ncc(Cl)c1CC1c2cc(OC3CNC3)ccc2C(=O)N1Cc1ccc2[nH]nnc2c1]

The title compound 437 was prepared according to the procedure described for compound 279 as a white solid (14 mg, 21%). MS (ESI) m/z 464 [M+H]⁺. ¹H NMR (400 MHz, CD₃OD) δ 7.84 (d, J=8.4 Hz, 1H), 7.81 (d, J=8.4 Hz, 1H), 7.76 (s, 11H), 7.51 (s, 1H), 7.35 (d, J=8.6 Hz, 1H), 7.04 (dd, J=8.5, 2.1 Hz, 1H), 6.07 (s, 1H), 5.49 (d, J=15.4 Hz, 1H), 5.07-5.01 (m, 1H), 4.68 (dd, J=8.8, 6.1 Hz, 1H), 4.51 (d, J=15.6 Hz, 1H), 4.45-4.36 (m, 2H), 4.10-4.03 (m, 2H), 3.61-3.54 (m, 1H), 3.53 (s, 3H), 2.94 (dd, J=14.8, 8.8 Hz, 1H).

The intermediate 437-1 was prepared as follows:

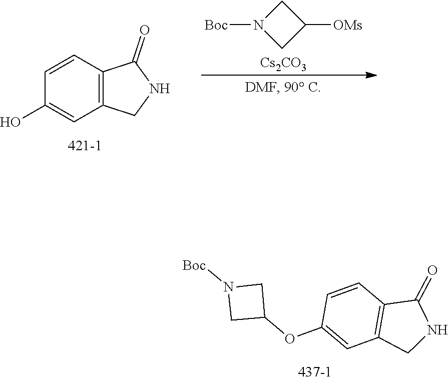

To a solution of 5-hydroxyisoindolin-1-one (300 mg, 2 mmol) and tert-butyl 3-((methylsulfonyl)oxy)azetidine-1-carboxylate (502 mg, 2 mmol) in DMF (5 mL) was added Cs₂CO₃ (1.3 g, 4 mmol). The resulting mixture was stirred for additional 12 h at 90° C. under N₂. The mixture was allowed to cool down to r.t. The resulting mixture was diluted with water (20 mL) and extracted with EtOAc (3×20 mL). The combined organic layers were washed with brine (10 mL), dried over anhydrous Na₂SO₄, and after filtration, the filtrate was concentrated under reduced pressure. The residue was purified by silica gel column chromatography eluting with PE/EA=1/1 to afford tert-butyl 3-((1-oxoisoindolin-5-yl)oxy)azetidine-1-carboxylate as a yellow solid. MS (ESI) m/z 249 [M−55]⁺.

### 2-((1H-benzo[d][1,2,3]triazol-5-yl)methyl)-3-((3-(azetidin-3-yl)-4-chloro-1-methyl-1H-pyrazol-5-yl)methyl)isoindolin-1-one (438)

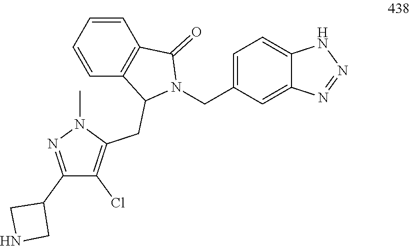 [SMILES: Cn1nc(C2CNC2)c(Cl)c1CC1c2ccccc2C(=O)N1Cc1ccc2[nH]nnc2c1]

The title compound 438 was prepared according to the procedure described for compound 279 as a white solid (28 mg, 44%). MS (ESI) m/z 448 [M+H]⁺. ¹H NMR (400 MHz, CD₃OD) δ 7.88-7.85 (m, 1H), 7.83 (d, J=8.4 Hz, 1H), 7.70 (s, 1H), 7.60-7.49 (m, 2H), 7.37 (dd, J=8.6, 1.2 Hz, 1H), 6.96-6.90 (m, 1H), 5.53 (d, J=15.4 Hz, 1H), 4.76 (dd, J=7.9, 6.2 Hz, 1H), 4.52 (d, J=15.5 Hz, 1H), 4.44-4.28 (m, 4H), 4.24-4.16 (m, 1H), 3.54 (s, 3H), 3.50 (dd, J=15.0, 6.4 Hz, 1H), 3.04 (dd, J=15.0, 7.9 Hz, 1H).

The intermediate 438-4 was prepared as follows:

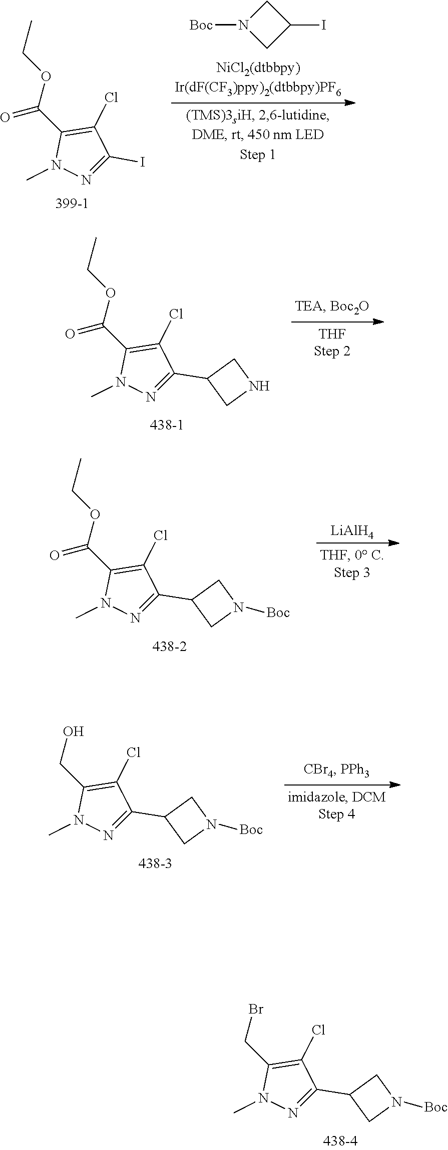

Step 1 was performed according to the procedure outlined for preparation of 198-1.

Step 2 was performed according to the procedure outlined for preparation of Int 12-1.

Step 3 was performed according to the procedure outlined for preparation of 263-2.

Step 4 was performed according to the procedure outlined for preparation of 164-3.

### (5s,8s)-8-((1-((3-bromopyridin-2-yl)methyl)-3-oxoisoindolin-2-yl)methyl)-1-oxa-3-azaspiro[4.5]decan-2-one (439) and (5r,8r)-8-((1-((3-bromopyridin-2-yl)methyl)-3-oxoisoindolin-2-yl)methyl)-1-oxa-3-azaspiro[4.5]decan-2-one (440)

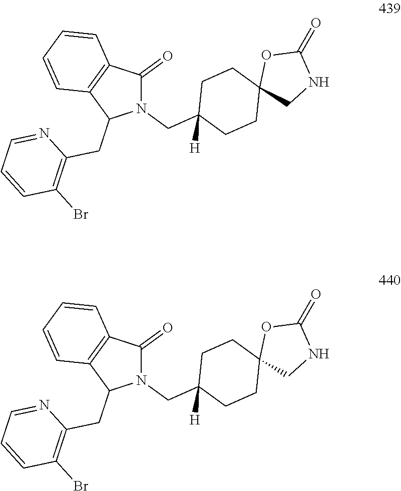 [SMILES: O=C1NC[C@]2(CC[C@H](CN3C(=O)c4ccccc4C3Cc3ncccc3Br)CC2)O1 ; O=C1NC[C@]2(CC[C@@H](CN3C(=O)c4ccccc4C3Cc3ncccc3Br)CC2)O1]

The title compounds 439 and 440 were prepared according to the procedure described for compound 270.

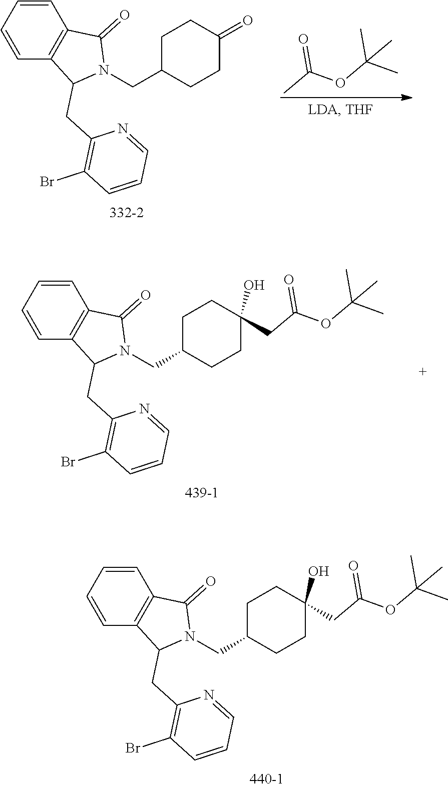

In the step mentioned above, 2 isomers were obtained: 439-1 and 440-1. One of the two isomers with RT=0.928 min (Diamonsll Plus 5 μm C18, 30*3.0 mm, 1.8 min 5-98% (0.02% NH₄0Ac)) and the other one of the two isomers with RT=0.960 min (Diamonsll Plus 5 μm C18, 30*3.0 mm, 1.8 min 5-98% (0.02% NH₄0Ac)). Compound synthesized by using 439-1/440-1 (RT=0.928 min) as a starting material was a white solid (39 mg). MS (ESI) m/z 470 [M+H]⁺. ¹H NMR (400 MHz, DMSO-d₆) δ 8.63-8.60 (m, 1H), 8.11 (d, J=8.0 Hz, 1H), 7.69-7.65 (m, 1H), 7.52-7.45 (m, 2H), 7.40 (d, J=6.8 Hz, 1H), 7.32 (dd, J=8.0, 4.4 Hz, 1H), 7.14-7.09 (m, 1H), 5.32-5.28 (m, 1H), 3.74-3.68 (m, 1H), 3.61-3.54 (m, 1H), 3.28-3.20 (m, 2H), 3.13 (s, 1H), 3.02-2.96 (m, 1H), 1.86-1.74 (m, 2H), 1.65-1.62 (m, 1H), 1.53-1.34 (m, 4H), 1.26-0.92 (m, 2H). Compound synthesized by using 439-1/440-1 (RT=0.960 min) as a starting material was a white solid (14 mg). MS (ESI) m/z 470 [M+H]⁺. ¹H NMR (400 MHz, DMSO-d₆) δ 8.62 (dd, J=4.8, 1.6 Hz, 1H), 8.11 (dd, J=8.4, 1.6 Hz, 1H), 7.68-7.66 (m, 1H), 7.52-7.45 (m, 2H), 7.41 (s, 1H), 7.32 (dd, J=8.0, 4.4 Hz, 1H), 7.13 (d, J=6.8 Hz, 1H), 5.31 (t, J=6.4 Hz, 1H), 3.73-3.67 (m, 1H), 3.57 (dd, J=14.8, 6.4 Hz, 1H), 3.28-3.20 (m, 3H), 2.99 (dd, J=14.0, 5.2 Hz, 1H), 1.80-1.43 (m, 7H), 1.12-0.89 (m, 2H).

### (2r,4r)-2-((1-((3-bromopyridin-2-yl)methyl)-3-oxoisoindolin-2-yl)methyl)-2-fluoro-5-oxa-7-azaspiro[3.4]octan-6-one (441) and (2s,4s)-2-((1-((3-bromopyridin-2-yl)methyl)-3-oxoisoindolin-2-yl)methyl)-2-fluoro-5-oxa-7-azaspiro[3.4]octan-6-one (442)

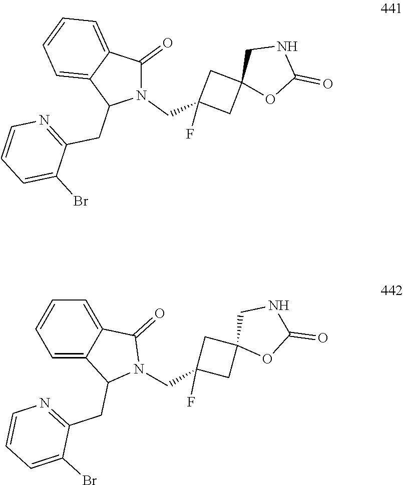 [SMILES: O=C1NC[C@]2(C[C@](F)(CN3C(=O)c4ccccc4C3Cc3ncccc3Br)C2)O1 ; O=C1NC[C@]2(C[C@@](F)(CN3C(=O)c4ccccc4C3Cc3ncccc3Br)C2)O1]

The title compounds 441 and 442 were prepared according to the procedure described for compound 270. After separation by prep-HPLC, two isomers were obtained. One of the two isomers was a white solid (0.4 mg, 2%), with RT=4.47 min, MS (ESI) m/z 460. The other one of the two isomers was a white solid (0.7 mg, 3%), with RT=4.63 min, MS (ESI) m/z 460.

The intermediate 441-6 was prepared as follows:

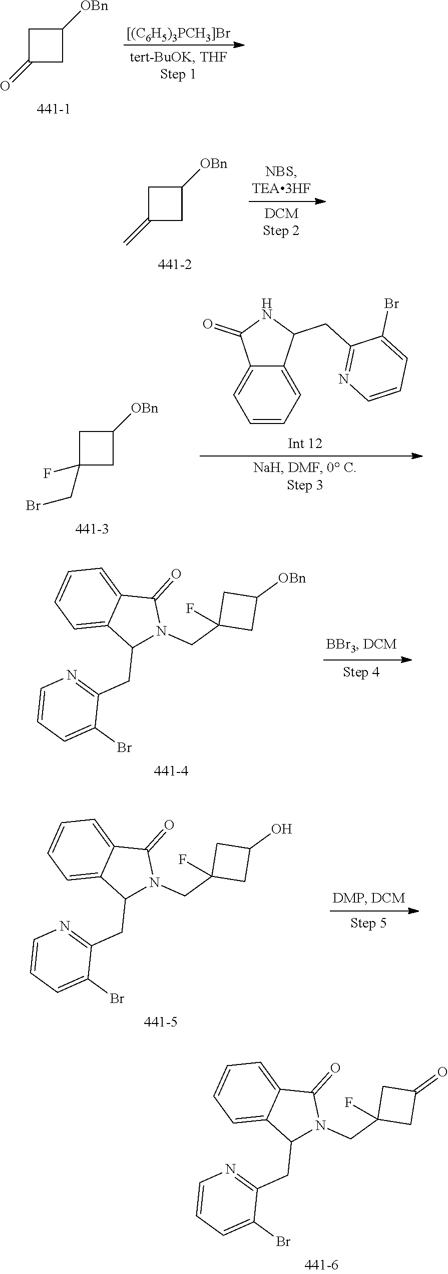

### Step 1. ((3-Methylenecyclobutoxy)methyl)benzene (441-2)

To a solution of methyltriphenylphosphonium bromide (15.20 g, 42.6 mmol) in THE (100 mL) was added potassium tert-butoxide (4.46 g, 39.7 mmol) and the mixture was stirred for 1 h. Then 3-benzyloxycyclobutanone (5 g, 28.4 mmol) was added and the mixture was stirred at 25° C. overnight. The reaction was monitored by TLC. Once completed, the solvent was removed under reduced pressure and the residue was dissolved in DCM, washed with water, dried over Na₂SO₄, concentrated and purified by column chromatograph (PE/EA=10/1) to yield the product (3.44 g, 70%). ¹H NMR (400 MHz, DMSO-d₆) δ 7.37-7.20 (m, 5H), 4.84 (tt, J=2.8, 2.0 Hz, 2H), 4.39 (s, 2H), 4.08 (p, J=6.5 Hz, 1H), 2.95-2.77 (m, 2H), 2.70-2.54 (m, 2H), 2.50 (p, J=1.9 Hz, 2H).

### Step 2. ((3-(Bromomethyl)-3-fluorocyclobutoxy)methyl)benzene (441-3)

To a solution of 441-2 (2.0 g, 11.5 mmol) and triethylamine trihydrofluoride (2.8 mL, 17.2 mmol) in DCM (30 mL) was added NBS (2.25 g, 12.6 mmol) at 0° C. and then the mixture was stirred for 5 h at r.t. The reaction was monitored by TLC. Once completed, solvent was removed under reduced pressure and the residue was dissolved in DCM, washed with water, dried over Na₂SO₄, concentrated and purified by column chromatograph (PE/EA=10/1) to yield the product (1.97 g, 62%). ¹H NMR (400 MHz, DMSO-d₆) δ 7.39-7.24 (m, 5H), 4.40 (d, J=2.2 Hz, 2H), 3.90-3.67 (m, 2H), 2.68-2.43 (m, 3H), 2.33-2.11 (m, 2H).

Step 3 was performed according to the procedure outlined for preparation of Int 5.

### Step 4. 3-[(3-bromo-2-pyridyl)methyl]-2-[(1-fluoro-3-hydroxy-cyclobutyl)methyl]isoindolin-1-one (441-5)

To a solution of 441-4 (123 mg, 0.25 mmol) in DCM (10 mL) was added BBr₃ (68 mg, 0.27 mmol) at −78° C. and the mixture was stirred for 1 h at 0° C. The reaction was monitored by LC/MS. Once completed, the reaction was quenched with water and extracted with EA. The organic layer was separated, dried over Na₂SO₄, concentrated and the residue was used directly in the next step.

### Step 5. 3-[(3-Bromo-2-pyridyl)methyl]-2-[(1-fluoro-3-oxo-cyclobutyl)methyl]isoindolin-1-one (441-6)

To a solution of 441-5 (130 mg, 0.32 mmol) in DCM (10 mL) was added DMP (408 mg, 0.96 mmol) and the mixture was stirred at 25° C. for 2 h. The reaction was monitored by LC/MS. Once completed, the reaction was quenched with Na₂S₂O₃ (aq.) and extracted with DCM. The organic layer was separated, dried over Na₂SO₄, concentrated and the residue was purified by column chromatograph (PE/EA=2/1) to yield the product (25 mg, 19%). MS (ESI) m/z 403 [M+H]⁺.

### N-(5-((1-((3-bromopyridin-2-yl)methyl)-3-oxoisoindolin-2-yl)methyl)pyrimidin-2-yl)acetamide (443)

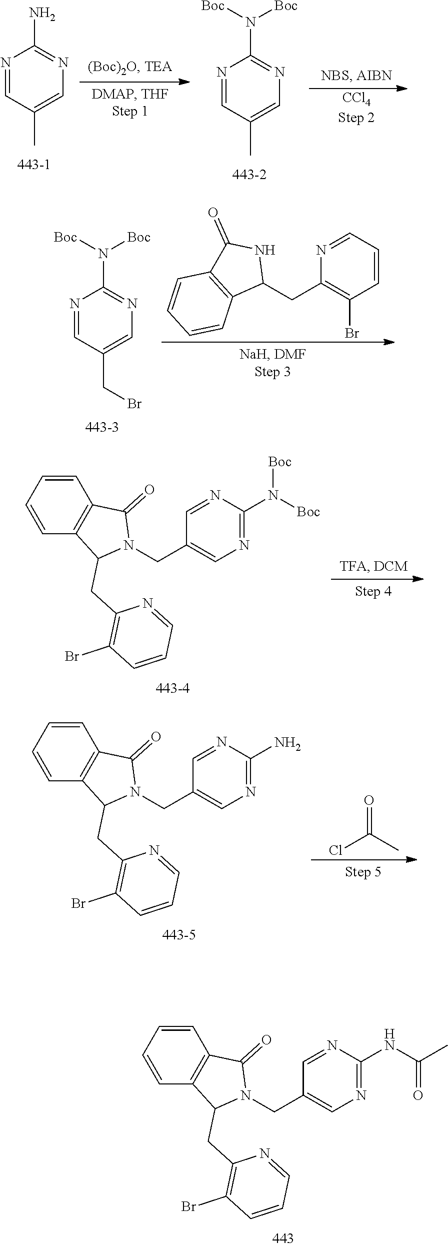

Step 1 was performed according to the procedure outlined for preparation of 23-2.

Step 2 was performed according to the procedure outlined for preparation of 222-1.

Step 3 was performed according to the procedure outlined for preparation of Int 5.

Step 4 was performed according to the procedure outlined for preparation of 23-4.

### Step 5. N-(5-((1-((3-bromopyridin-2-yl)methyl)-3-oxoisoindolin-2-yl)methyl) pyrimidin-2-yl)acetamide (443)

To a solution of 443-5 (50 mg, 0.12 mmol) in THF (5 mL) was added acetyl chloride (0.01 mL, 0.12 mmol) at 0° C. under N₂. The mixture was stirred for 1 h at 0° C. The mixture was concentrated in vacuo and purified by prep-HPLC to give 443 (1 mg, 1.8%) as a white solid. MS (ESI) m/z 452 [M+H]⁺.

### 3-((2-((1H-benzo[d][1,2,3]triazol-5-yl)methyl)-3-oxoisoindolin-1-yl)methyl)-4-methylpicolinamide (444)

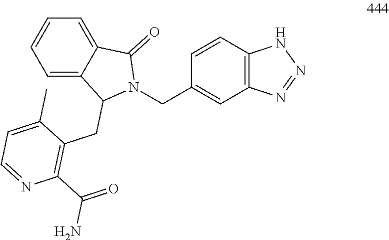 [SMILES: Cc1ccnc(C(N)=O)c1CC1c2ccccc2C(=O)N1Cc1ccc2[nH]nnc2c1]

The title compound 444 was prepared according to the procedure described for compound 279 as a white solid (3 mg, 3.6%). MS (ESI) m/z 413 [M+H]⁺. ¹H NMR (400 MHz, CD₃OD) δ 8.46 (d, J=4.8 Hz, 1H), 7.86 (d, J=7.6 Hz, 1H), 7.81 (d, J=8.7 Hz, 11H), 7.62 (s, 1H), 7.51 (t, J=7.5 Hz, 1H), 7.46-7.33 (m, 3H), 6.66 (d, J=7.6 Hz, 1H), 5.39 (d, J=15.3 Hz, 1H), 5.00 (t, J=7.6 Hz, 1H), 4.51 (d, J=15.3 Hz, 1H), 4.01 (dd, J=13.3, 7.3 Hz, 1H), 3.233-3.13 (m, 1H), 1.99 (s, 3H).

The intermediate 444-5 was prepared as follows:

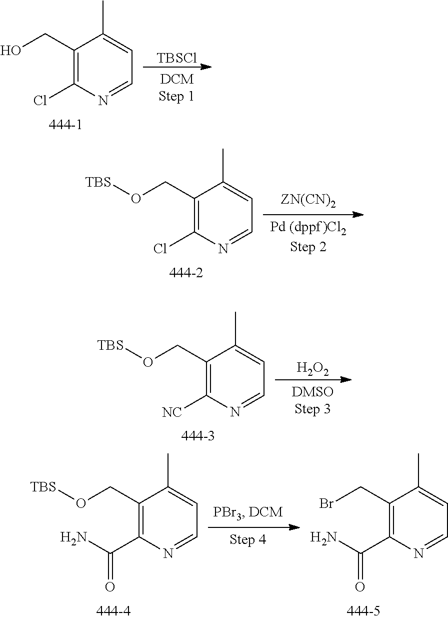

Step 1 was performed according to the procedure outlined for preparation of 360-3.

Step 2 was performed according to the procedure outlined for preparation of 278-4.

### Step 3. 3-(((tert-butyldimethylsilyl)oxy)methyl)-4-methylpicolinamide (444-4)

To a solution of 444-3 (8 g, 0.03 mol) and K₂CO₃ (8.43 g, 0.061 mol) in DMSO (160 mL) was added H₂O₂(80 mL) dropwise at r.t. The resulting mixture was stirred for 1 h. The mixture was quenched with 500 mL water and 50 g Na₂S2O₃ and extracted with EA (300 mL+200 mL). The EA phases were combined and washed with 50 mL*2 half saturated saline twice, and the EA phase was dried with Na₂SO₄. The solution was filtered and the filtrate was evaporated to obtain 444-4 (7.5 g, 87%) as a black solid. MS (ESI) m/z 281 [M+H]⁺.

### Step 4 was performed according to the procedure outlined for preparation of 263-3. 2-((1-((4-chloro-1-methyl-1H-pyrazol-5-yl)methyl)-1-methyl-3-oxoisoindolin-2-yl)methyl)-5-oxa-7-azaspiro[3.4]octan-6-one (445)

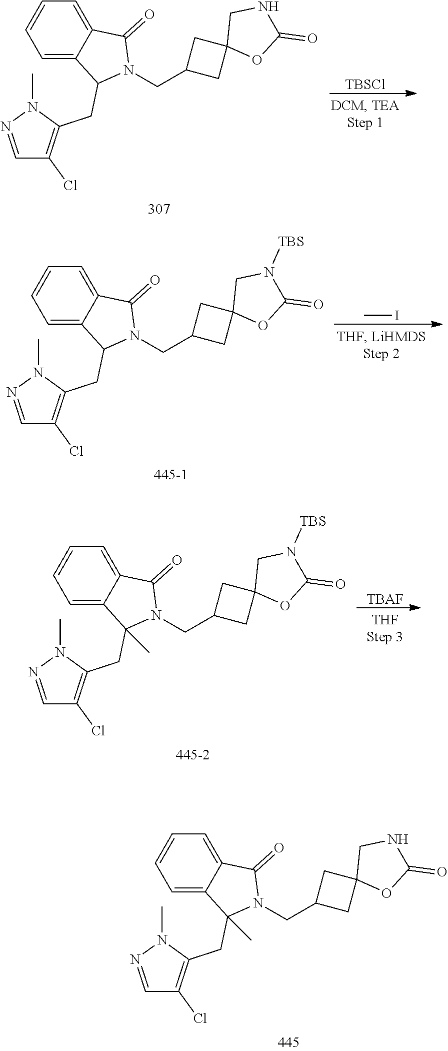

### Step 1. 7-(tert-butyldimethylsilyl)-2-((1-((4-chloro-1-methyl-1H-pyrazol-5-yl)methyl)-3-oxoisoindolin-2-yl)methyl)-5-oxa-7-azaspiro[3.4]octan-6-one (445-1)

To a solution of 307 (400 mg, 0.99 mmol) in DCM (10 mL) were added TEA (0.42 mL, 2.99 mmol) and TBSCl (150 mg, 0.99 mmol) under N₂. The mixture was stirred for 12 h, then diluted with DCM, and washed with water. The organic layer was separated, dried over anhydrous Na₂SO₄ and concentrated under vacuum. The residue was purified by silica gel column chromatography (PE/EA=1/2) to give 445-1 (480 mg, 93.4%) as a yellow solid. MS (ESI) m/z 515 [M+H]⁺.

Step 2 was performed according to the procedure outlined for preparation of Int 12-2.

### Step 3. 2-((1-((4-chloro-1-methyl-1H-pyrazol-5-yl)methyl)-1-methyl-3-oxoisoindolin-2-yl)methyl)-5-oxa-7-azaspiro[3.4]octan-6-one (445)

To a solution of 445-2 (200 mg, 0.37 mmol) in THE (2 mL) was added TBAF (1 mL). The mixture was stirred for 1 h. The solution was taken in EtOAc, and washed with water. The organic layer was separated, dried over anhydrous Na₂SO₄ and concentrated. The residue was purified by prep HPLC to give 445 (63 mg, 38%) as a white solid. MS (ESI) m/z 415 [M+H]⁺. ¹H NMR (400 MHz, CDCl₃) δ 7.82 (d, J=6.5 Hz, 1H), 7.51-7.39 (m, 3H), 6.66 (d, J=6.8 Hz, 1H), 5.02 (s, 1H), 3.86 (dd, J=14.2, 6.2 Hz, 1H), 3.62 (s, 2H), 3.42-3.32 (m, 2H), 2.98 (s, 3H), 2.67-2.54 (m, 2H), 2.52-2.32 (m, 4H), 1.65 (s, 3H).

### 3-((3-oxo-2-((6-oxo-5-oxa-7-azaspiro[3.4]octan-2-yl)methyl)isoindolin-1-yl)methyl)pyridine-2,4-dicarbonitrile (446)

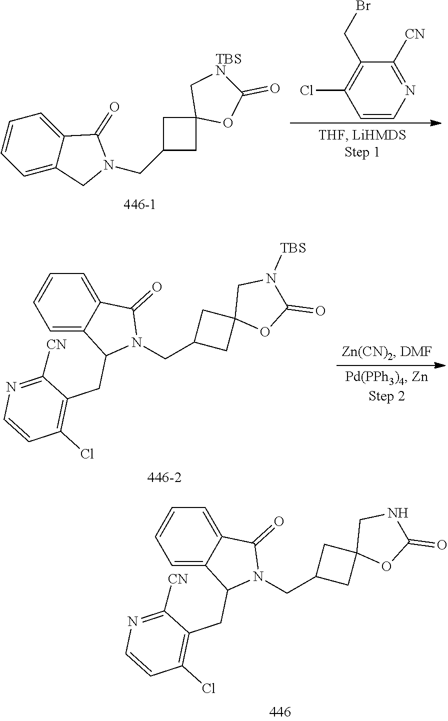

Step 1 was performed according to the procedure outlined for preparation of Int 12-2.

### Step 2. 3-((3-oxo-2-((6-oxo-5-oxa-7-azaspiro[3.4]octan-2-yl)methyl)isoindolin-1-yl)methyl)pyridine-2,4-dicarbonitrile (446)

To a solution of 446-2 (400 mg, 0.74 mmol) in DMF (8 mL) were added Zn (10 mg, 0.14 mmol), Zn(CN)₂ (261 mg, 2.23 mmol), Pd(PPh₃)₄(86 mg, 0.07 mmol) under N₂. The mixture was stirred at 120′C for 14 h. The mixture was diluted with EtOAc and washed with water. The organic layer was separated, dried over anhydrous Na₂SO₄ and concentrated under vacuum. The residue was purified by prep HPLC to give 446 (64 mg, 20%) as a yellow solid. MS (ESI) m/z 414 [M+H]⁺. ¹H NMR (400 MHz, DMSO-d₆) δ 8.96 (d, J=5.0 Hz, 1H), 8.29 (d, J=4.9 Hz, 1H), 7.71 (dd, J=6.6, 1.5 Hz, 1H), 7.55-7.42 (m, 3H), 6.64 (d, J=7.0 Hz, 1H), 5.02 (dd, J=9.3, 4.9 Hz, 1H), 4.11-3.94 (m, 2H), 3.50 (s, 2H), 3.45 (dd, J=14.3, 5.5 Hz, 1H), 2.86 (dd, J=14.0, 9.4 Hz, 11H), 2.43-2.23 (m, 4H), 2.17-2.05 (m, 11H).

The intermediate 446-1 was prepared as follows:

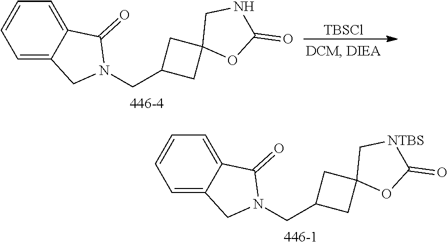

446-4 was prepared according to the procedure described for preparation of 307.

446-1 was prepared according to the procedure described for preparation of 445-1.

### 2-bromo-5-(4-hydroxybenzyl)-4,5-dihydro-6H-pyrrolo[3,4-d]thiazol-6-one (447)

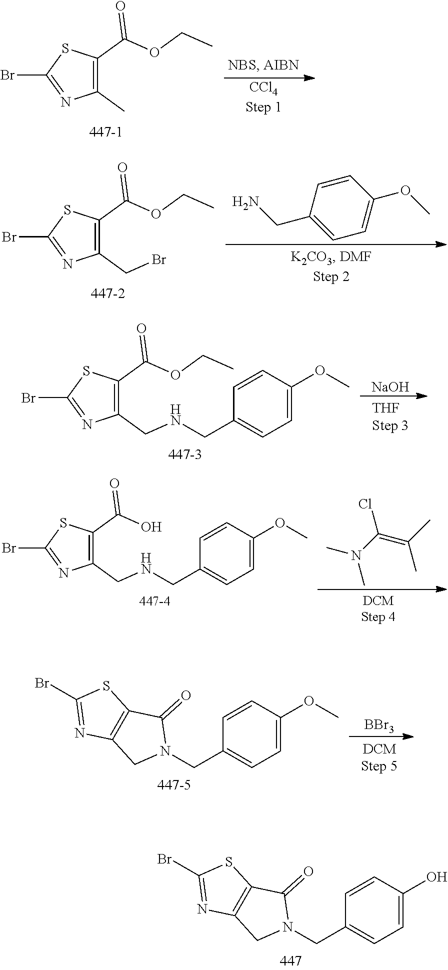

Step 1 was performed according to the procedure outlined for preparation of 222-1.

### Step 2. ethyl 2-bromo-4-(((4-methoxybenzyl)amino)methyl)thiazole-5-carboxylate (447-3)

To a solution of 447-2 (1.0 g, 3.06 mmol) in DMF (20 mL) were added K₂CO₃ (845 mg, 6.12 mmol) and (4-methoxyphenyl)methanamine (503 mg, 3.67 mmol) at r.t. The mixture was stirred for 2 h at r.t. The mixture was diluted with EtOAc and washed with water. The organic layer was separated, dried over anhydrous Na₂SO₄ and purified by silica gel flash column chromatography (PE/EA=1/3) to afford 447-3 (500 mg, 42.6%) as a yellow solid. MS (ESI) m/z 385 [M+H]⁺.

Step 3 was performed according to the procedure outlined for preparation of 430-4.

### Step 4. 2-bromo-5-(4-methoxybenzyl)-4,5-dihydro-6H-pyrrolo[3,4-d]thiazol-6-one (447-5)

To a solution of 447-4 (100 mg, 0.28 mmol) in DCM (5 mL) was added 1-chloro-N,N,2-trimethylprop-1-en-1-amine (75 mg, 0.56 mmol) at 0° C. under N₂. The mixture was stirred for 30 min at 0° C. and then water was added, extracted with DCM. The organic layer was separated, dried over anhydrous Na₂SO₄ and concentrated under vacuum to give 447-5 (70 mg) as a yellow solid, which was used in the next step without further purification. MS (ESI) m/z 339 [M+H]⁺.

Step 5 was performed according to the procedure outlined for step 2 of general procedure D to afford 447 as a white solid (7 mg, 10.3%). MS (ESI) m/z 325 [M+H]⁺. ¹H NMR (400 MHz, DMSO-d₆) δ 9.39 (s, 1H), 7.10 (d, J=8.5 Hz, 2H), 6.72 (d, J=8.5 Hz, 2H), 4.55 (s, 2H), 4.44 (s, 2H).

### 2-((1H-benzo[d][1,2,3]triazol-5-yl)methyl)-3-((2-bromo-5-chlorothiazol-4-yl)methyl)isoindolin-1-one (448)

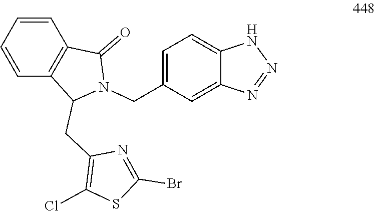 [SMILES: O=C1c2ccccc2C(Cc2nc(Br)sc2Cl)N1Cc1ccc2[nH]nnc2c1]

The title compound 448 was prepared according to the procedure described for compound 279 as a white solid (6 mg, 14%). MS (ESI) m/z 474 [M+H]⁺. ¹H NMR (400 MHz, CDCl₃) δ 7.87-7.80 (m, 1H), 7.76 (d, J=8.5 Hz, 1H), 7.59 (s, 1H), 7.49-7.37 (m, 2H), 7.21 (d, J=8.8 Hz, 1H), 7.10 (d, J=7.0 Hz, 1H), 5.34 (d, J=15.6 Hz, 1H), 4.88 (t, J=6.1 Hz, 1H), 4.50 (d, J=15.6 Hz, 1H), 3.18 (dd, J=14.8, 6.0 Hz, 1H), 3.03 (dd, J=14.9, 6.2 Hz, 1H).

### 3-((5-fluoro-3-oxo-2-((6-oxo-5-oxa-7-azaspiro[3.4]octan-2-yl)methyl)isoindolin-1-yl)methyl)-4-methylpicolinonitrile (449)

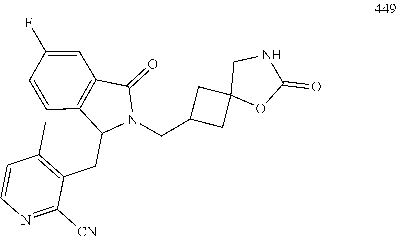 [SMILES: Cc1ccnc(C#N)c1CC1c2ccc(F)cc2C(=O)N1CC1CC2(CNC(=O)O2)C1]

The title compound 449 was prepared according to the procedure described for compound 308 (200 mg, 71.9%) as a white solid. MS (ESI) m/z 421 [M+H]⁺. ¹H NMR (400 MHz, CDCl₃) δ 8.58 (d, J=4.9 Hz, 1H), 7.54 (dd, J=7.4, 2.5 Hz, 1H), 7.38 (d, J=4.9 Hz, 1H), 7.03 (td, J=8.6, 2.5 Hz, 1H), 6.32 (dd, J=8.4, 4.3 Hz, 1H), 5.44 (s, 1H), 4.89 (dd, J=10.2, 5.5 Hz, 1H), 4.22 (dd, J=14.2, 7.7 Hz, 1H), 3.70 (dd, J=13.4, 5.6 Hz, 1H), 3.62 (s, 2H), 3.36 (dd, J=14.3, 5.7 Hz, 1H), 2.75 (dd, J=13.7, 10.1 Hz, 1H), 2.58-2.31 (m, 5H), 2.01 (s, 3H).

### 2-((5-bromo-3-((4-chloro-1-methyl-1H-pyrazol-5-yl)methyl)-1-oxoisoindolin-2-yl)methyl)-5-oxa-7-azaspiro[3.4]octan-6-one (450)

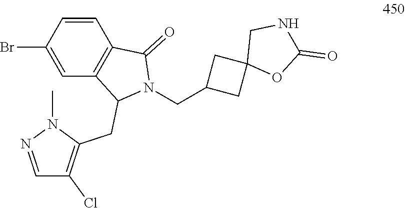 [SMILES: Cn1ncc(Cl)c1CC1c2cc(Br)ccc2C(=O)N1CC1CC2(CNC(=O)O2)C1]

The title compound 450 was prepared according to the procedure described for compound 307 (1.4 g, 59.7%) as a white solid. MS (ESI) m/z 479 [M+H]⁺. ¹H NMR (400 MHz, CDCl₃) δ 7.71 (d, J=8.1 Hz, 1H), 7.61 (dd, J=8.1, 1.6 Hz, 1H), 7.53 (s, 1H), 6.74 (s, 1H), 5.75 (s, 1H), 4.80 (dd, J=9.4, 5.7 Hz, 1H), 4.17 (dd, J=14.4, 5.2 Hz, 1H), 3.60 (s, 2H), 3.54 (s, 3H), 3.43 (dd, J=14.5, 5.7 Hz, 1H), 3.15 (dd, J=14.5, 6.4 Hz, 1H), 2.64 (dd, J=14.5, 9.4 Hz, 1H), 2.46-2.26 (m, 5H).

### 2-((1-((4-chloro-3-methoxy-1-methyl-1H-pyrazol-5-yl)methyl)-3-oxoisoindolin-2-yl)methyl)-5-oxa-7-azaspiro[3.4]octan-6-one (451)

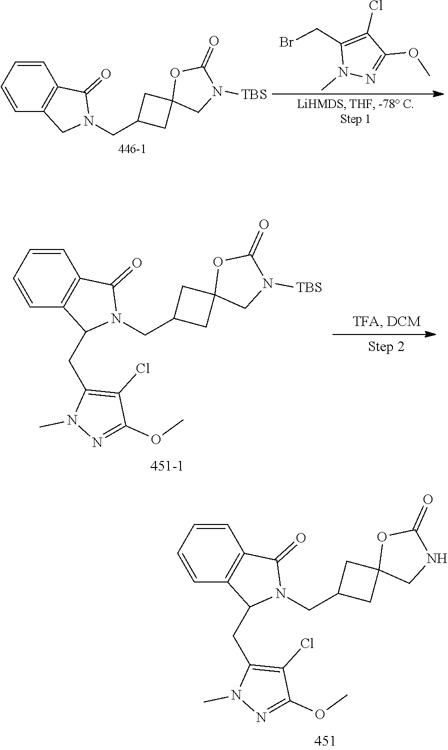

Step 1 was performed according to the procedure outlined for preparation of Int 12-2.

### Step 2. 2-((1-((4-chloro-3-methoxy-1-methyl-1H-pyrazol-5-yl)methyl)-3-oxoisoindolin-2-yl)methyl)-5-oxa-7-azaspiro[3.4]octan-6-one (451)

To a solution of 451-1 (380 mg, 0.7 mmol) in DCM (3 mL) was added TFA (3 mL) at 0° C. The mixture was stirred for 2 h at r.t, and the concentrated. The residue was purified by Prep-HPLC to give 451 as a white solid (83 mg, 28%). MS (ESI) m/z 431 [M+H]+. 1H NMR (400 MHz, CDCl₃) δ 7.86 (dd, J=6.4, 1.6 Hz, 1H), 7.52-7.42 (m, 2H), 6.69 (d, J=6.8 Hz, 1H), 4.96 (s, 1H), 4.83 (dd, J=9.8, 5.4 Hz, 1H), 4.21 (dd, J=14.4, 5.4 Hz, 1H), 4.00 (s, 3H), 3.51 (s, 2H), 3.42 (dd, J=14.4, 5.5 Hz, 1H), 3.33 (s, 3H), 3.22 (dd, J=14.4, 6.4 Hz, 1H), 2.52 (dd, J=14.3, 9.8 Hz, 1H), 2.46-2.29 (m, 5H).

### 2-(3-(1H-imidazol-4-yl)prop-2-yn-1-yl)-3-((4-chloro-1-methyl-1H-pyrazol-5-yl)methyl)insoindolin-1-one (450)

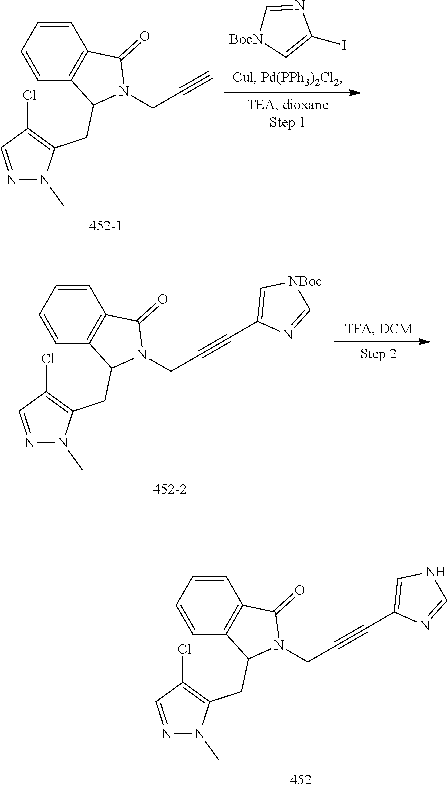 [SMILES: C#CCN1C(=O)c2ccccc2C1Cc1c(Cl)cnn1C ; CC(C)(C)OC(=O)n1cnc(I)c1 ; Cn1ncc(Cl)c1CC1c2ccccc2C(=O)N1CC#Cc1cn(C(=O)OC(C)(C)C)cn1 ; Cn1ncc(Cl)c1CC1c2ccccc2C(=O)N1CC#Cc1c[nH]cn1]

The intermediate 452-1 was prepared according to the procedure outlined for 222-2.

Step 1 was performed according to the procedure outlined for preparation of 417-2.

### Step 2. 2-(3-(1H-imidazol-4-yl)prop-2-yn-1-yl)-3-((3-bromopyridin-2-yl)methyl) isoindolin-1-one (452)

TFA (1 mL) was added to a stirred solution of 452-2 (20 mg, 0.043 mmol) in DCM (1 mL). The mixture was stirred for 1 h. The mixture was concentrated in vacuo and purified by prep-HPLC to give 452 (5 mg, 31.8%) as a white solid. MS (ESI) m/z 366 [M+H]⁺. ¹H NMR (400 MHz, DMSO-d₆) δ 12.29 (s, 1H), 7.72 (dd, J=6.8, 1.5 Hz, 1H), 7.64 (s, 1H), 7.59-7.48 (m, 3H), 7.42 (s, 1H), 6.97 (d, J=7.3 Hz, 1H), 5.08 (dd, J=8.4, 5.8 Hz, 1H), 4.95 (d, J=17.9 Hz, 1H), 4.29 (d, J=18.0 Hz, 1H), 3.74 (s, 3H), 3.58 (dd, J=14.8, 5.8 Hz, 1H), 3.04 (dd, J=14.8, 8.4 Hz, 11H).

### 2-(3-(1H-pyrazol-4-yl)prop-2-yn-1-yl)-3-((4-chloro-1-methyl-1H-pyrazol-5-yl)methyl)isoindolin-1-one (453)

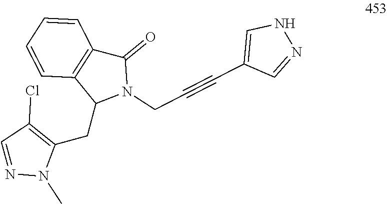 [SMILES: Cn1ncc(Cl)c1CC1c2ccccc2C(=O)N1CC#Cc1cn[nH]c1]

The title compound 453 was prepared according to the procedure described for compound 452 as a white solid (6 mg, 29.3%). MS (ESI) m/z 366 [M+H]⁺. ¹H NMR (400 MHz, DMSO-d₆) δ 13.10 (s, 1H), 8.01 (s, 1H), 7.72 (dd, J=6.9, 1.4 Hz, 1H), 7.63 (s, 1H), 7.59-7.48 (m, 3H), 6.98 (d, J=7.4 Hz, 1H), 5.10 (dd, J=8.2, 6.0 Hz, 1H), 4.95 (d, J=18.0 Hz, 1H), 4.25 (d, J=17.9 Hz, 1H), 3.73 (s, 3H), 3.55 (dd, J=14.9, 6.0 Hz, 1H), 3.05 (dd, J=14.8, 8.3 Hz, 1H).

### 4-methyl-3-((3-oxo-2-((4,5,6,7-tetrahydro-1H-benzo[d][1,2,3]triazol-5-yl)methyl)isoindolin-1-yl)methyl)picolinonitrile (454)

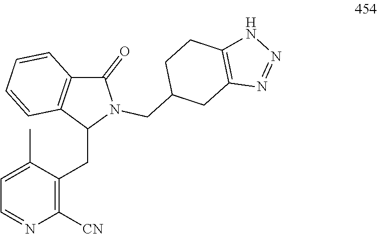 [SMILES: Cc1ccnc(C#N)c1CC1c2ccccc2C(=O)N1CC1CCc2[nH]nnc2C1]

The title compound 454 was prepared according to the procedure described for compound 332 as a white solid (5 mg, 15.6%). MS (ESI) m/z 399 [M+H]⁺. ¹H NMR (400 MHz, CD₃OD) δ 8.53 (d, J=4.8 Hz, 1H), 7.85 (d, J=7.6 Hz, 1H), 7.56-7.52 (m, 2H), 7.46-7.42 (m, 1H), 6.52 (dd, J=8.0, 3.6 Hz, 1H), 5.16-5.10 (m, 1H), 4.20-4.12 (m, 1H), 3.95-3.89 (m, 1H), 3.53-3.42 (m, 1H), 2.95-2.37 (m, 6H), 2.22 (s, 3H), 2.14-1.94 (m, 1H), 1.73-1.55 (m, 1H).

The intermediate 454-4 was prepared as follows:

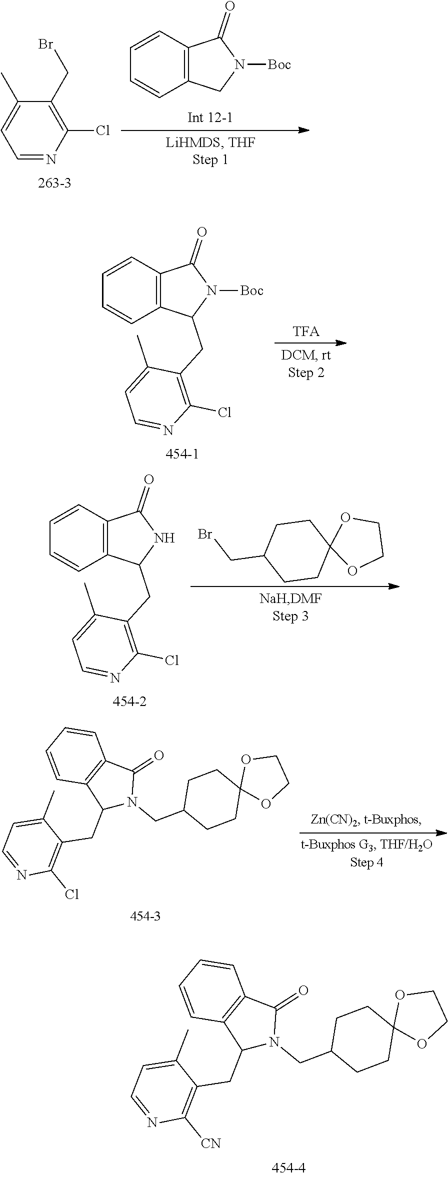

Step 1 was performed according to the procedure outlined for preparation of Int 12-2.

Step 2 was performed according to the procedure outlined for preparation of Int 12.

Step 3 was performed according to the procedure outlined for preparation of 270-2.

Step 4 was performed according to the procedure outlined for preparation of 308-3.

### 5-((1r,3r)-3-(1-((4-chloro-1-methyl-1H-pyrazol-5-yl)methyl)-3-oxoisoindolin-2-yl)cyclobutyl)oxazol-2(3H)-one (455)

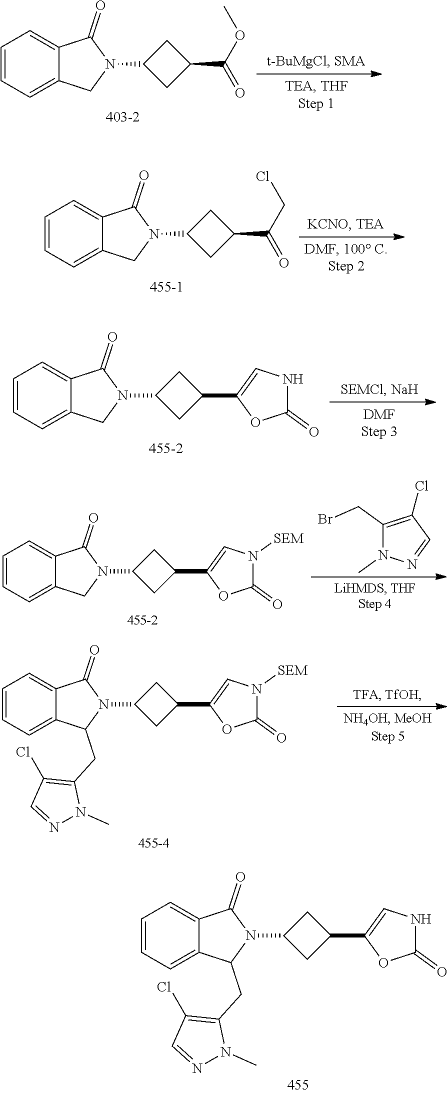

### Step 1. 2-((1r,3r)-3-(2-chloroacetyl)cyclobutyl)isoindolin-1-one (455-1)

To a solution of 403-2 (1.5 g, 6.1 mmol), SMA (1.1 g, 9.1 mmol) and TEA (0.93 g, 9.1 mmol) in THF (100 mL) was added t-BuMgCl (37 mL, 1 M in THF) at 0′C for 3 h. The mixture was stirred at r.t. for 5 h. The reaction mixture was poured into ice water (50 mL), extracted with EA (50 mL*2), dried over anhydrous Na₂SO₄, filtered and concentrated under vacuum. The residue was purified by HPLC to give 455-1 (2.4 g, 75%) as a yellow solid. MS (ESI) m/z 264 [M+H]⁺.

### Step 2. 5-((1r,3r)-3-(1-oxoisoindolin-2-yl)cyclobutyl)oxazol-2(3H)-one (455-2)

A mixture of 455-1 (1.2 g, 4.6 mmol), KCNO (739 mg, 9.1 mmol) and TEA (1.3 g, 12.9 mmol) in DMF (30 mL) was stirred at 100° C. for 3 h. The mixture was filtered, concentrated and purified by HPLC to give 455-2 (240 mg, 17%) as a colorless oil. MS (ESI) m/z 271 [M+H]⁺.

Step 3 was performed according to the procedure outlined for preparation of Int 1-3.

Step 4 was performed according to the procedure outlined for preparation of Int 12-2.

### Step 5. 5-((1r,3r)-3-(1-((4-chloro-1-methyl-1H-pyrazol-5-yl)methyl)-3-oxoisoindolin-2-yl)cyclobutyl)oxazol-2(3H)-one (455)

A mixture of 455-4 (50 mg) in TFA (2 mL) and TfOH (0.1 mL) was stirred at 50° C. for 30 min. The mixture was concentrated and added NH₃H₂O/MeOH (2 mL/2 mL) at r.t. The mixture was stirred at r.t. for 1 h. The mixture was concentrated and purified by prep-HPLC to give 455 (3 mg, 8%) as a white solid. MS (ESI) m/z 399 [M+H]⁺. ¹H NMR (400 MHz, CD₃OD) δ 7.77-7.75 (m, 1H), 7.52-7.49 (m, 3H), 6.74-6.72 (m, 1H), 6.63 (d, J=1.6 Hz, 1H), 5.10-5.07 (m, 1H), 4.58-4.50 (m, 1H), 3.57 (s, 3H), 3.51-3.39 (m, 2H), 3.19-3.04 (m, 2H), 2.87 (dd, J=14.8, 4.8 Hz, 1H), 2.60-2.55 (m, 1H), 2.48-2.42 (m, 1H).

### 2-(2-((1H-1,2,3-triazol-4-yl)amino)ethyl)-3-((3-bromopyridin-2-yl)methyl)isoindolin-1-one (456)

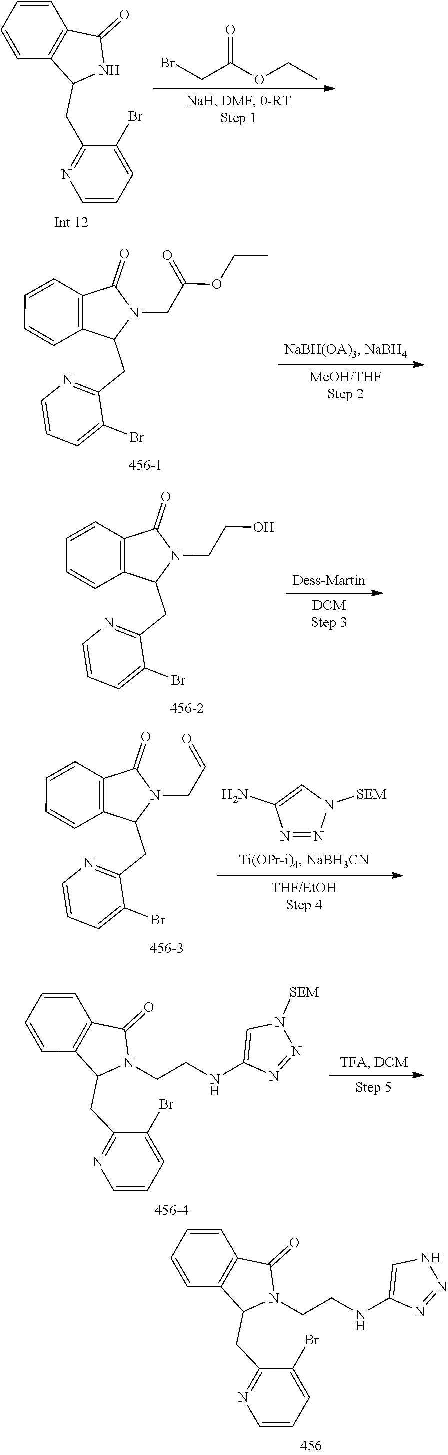

Step 1 was performed according to the procedure outlined for preparation of Int 5.

Step 2 was performed according to the procedure outlined for preparation of 164-2.

Step 3 was performed according to the procedure outlined for preparation of 403-5.

### Step 4: 3-((3-bromopyridin-2-yl)methyl)-2-(2-((1-((2-(trimethylsilyl)ethoxy)methyl)-1H-1,2,3-triazol-4-yl)amino)ethyl)isoindolin-1-one (456-4)

A mixture of 456-3 (50 mg, 0.15 mmol), 1-((2-(trimethylsilyl)ethoxy)methyl)-1H-1,2,3-triazol-4-amine (32 mg, 0.15 mmol) and Ti(OPr-i)₄ (128 mg, 0.45 mmol) in THF (5 mL) was stirred at r.t. for 1 h. NaBH₃CN (15 mg, 0.23 mmol) and EtOH (5 mL) were added in an ice-bath. The mixture was stirred at r.t. overnight. Then the mixture was added water (20 mL) and extracted with EA (20 mL*2), washed with brine (20 mL), dried over anhydrous Na₂SO₄, filtered and concentrated. The residue was purified by FCC (PE/EA=1/2) to give 456-4 (30 mg, 36%) as a yellow oil. MS (ESI) m/z 543 [M+H]⁺.

Step 5 was performed according to the procedure outlined for preparation of compound 7 to give 456 as a white solid (1 mg, 4%). MS (ESI) m/z 413 [M+H]⁺. ¹H NMR (400 MHz, CD₃OD) δ 8.51-8.47 (m, 1H), 8.00 (dd, J=8.4, 1.2 Hz, 1H), 7.77-7.71 (m, 1H), 7.48-7.45 (m, 2H), 7.22 (dd, J=8.4, 4.8 Hz, 1H), 7.01-6.99 (m, 2H), 5.42 (t, J=6.0 Hz, 1H), 4.16-4.10 (m, 1H), 3.69 (dd, J=14.4, 5.6 Hz, 1H), 3.54-3.44 (m, 3H), 3.30-3.21 (m, 1H).

### 3-((4-chloro-1-methyl-1H-pyrazol-5-yl)methyl)-2-((1,4,5,6-tetrahydrocyclopenta[d][1,2,3]triazol-4-yl)methyl)isoindolin-1-one (457)

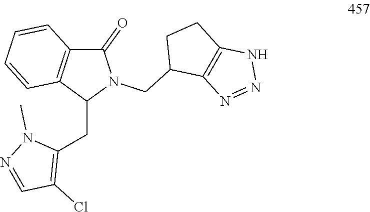 [SMILES: Cn1ncc(Cl)c1CC1c2ccccc2C(=O)N1CC1CCc2[nH]nnc21]

The title compound 457 was prepared according to the procedure described for compound 401-1 as a yellow solid (10 mg, 8%). MS (ESI) m/z 383 [M+H]⁺. ¹H NMR (400 MHz, CD₃OD) δ 7.83-7.73 (m, 1H), 7.55-7.45 (m, 3H), 6.83-6.80 (m, 0.4H), 6.71-6.69 (m, 0.6H), 5.14-5.10 (m, 0.4H), 4.99-4.96 (m, 0.6H), 4.29-4.22 (m, 1H), 3.71-3.54 (m, 5H), 3.52-3.47 (m, 1H), 3.07-2.67 (m, 4H), 2.66-2.34 (m, 1H).

### (E)-5-(3-(1-((3-bromopyridin-2-yl)methyl)-3-oxoisoindolin-2-yl)prop-1-en-1-yl)-1,3,4-oxadiazol-2(3H)-one (458)

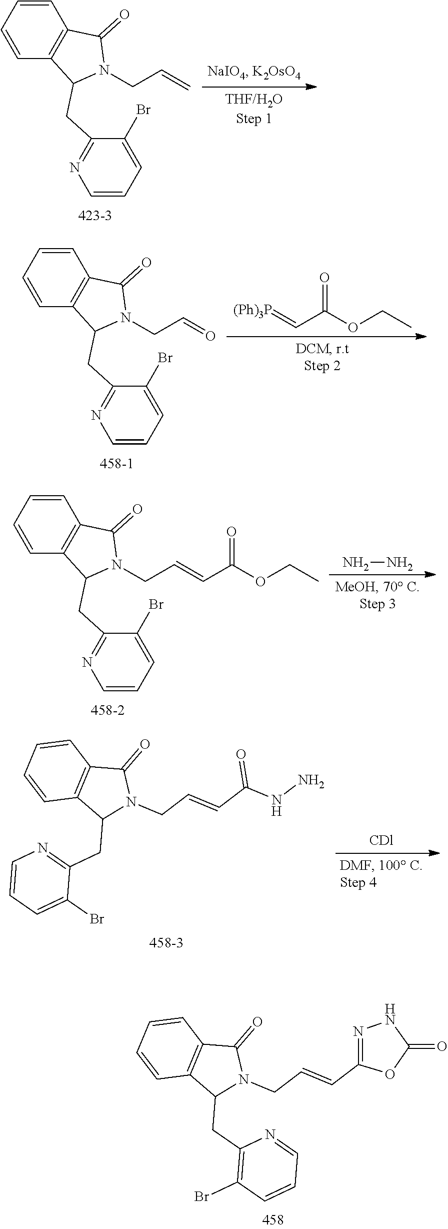

Step 1 was performed according to the procedure outlined for preparation of 381-6.

### Step 2. ethyl (E)-4-(1-((3-bromopyridin-2-yl)methyl)-3-oxoisoindolin-2-yl)but-2-enoate (458-2)

To a solution of 458-1 in dried DCM (10 mL) was added ethyl 2-(triphenyl-15-phosphaneylidene)acetate (3.6 g, 10.4 mmol), and the mixture was then stirred for 16 h at 25° C. The mixture was diluted with water (30 mL) and extracted with ethyl acetate (10 mL*2), The combined organic layer was washed with water (10 mL) and brine (10 mL), dried over Na₂SO₄, filtered, and concentrated in vacuo. The residue was purified by column chromatography on silica gel (eluting (PE/EA=2/1 to 1/1) to give 458-2 (780 mg, 45%) as a brown oil. MS (ESI) m/z 415 [M+H]⁺.

### Step 3. (E)-4-(1-((3-bromopyridin-2-yl)methyl)-3-oxoisoindolin-2-yl)but-2-enehydrazide (458-3)

To a solution of 458-2 (780 mg, 1.8 mmol) in dried MeOH (10 mL) was added N₂H₄ f-120 (360 mg, 7.2 mmol), the mixture was stirred at 80° C. for 30 min. The mixture was diluted with water (10 mL) and extracted with ethyl acetate (10 mL*2). The combined organic layer was washed with water (2*10 mL) and brine (2*10 mL), dried over Na₂SO₄, filtered, and concentrated in vacuo. The residue was purified by column chromatography on silica gel (eluting (PE/EA=2/1 to 1/1) to give 458-3 (100 mg, 45%) as a white solid. MS (ESI) m/z 401 [M+H]⁺.

### Step 4. (E)-5-(3-(1-((3-bromopyridin-2-yl)methyl)-3-oxoisoindolin-2-yl)prop-1-en-1-yl)-1,3,4-oxadiazol-2(3H)-one (458)

To a solution of 458-3 (100 mg, 0.25 mmol) in dried DMF (5 mL) was added CDI (81 mg, 0.5 mmol). The mixture was stirred at 100′C for 2 h. The mixture was diluted with water (5 mL) and extracted with ethyl acetate (2*10 mL). The combined organic layer was washed with water (10 mL) and brine (10 mL), dried over Na₂SO₄, filtered, and concentrated in vacuo. The residue was purified by prep-HPLC to give 458 (10 mg, 12%) as a white solid. MS (ESI) m/z 427 [M+H]+¹H NMR (400 MHz, CDCl₃) δ 8.61 (d, J=3.8 Hz, 1H), 7.89 (t, J=6.4 Hz, 2H), 7.48-7.36 (m, 2H), 7.16 (dd, J=7.8, 4.8 Hz, 1H), 6.87 (d, J=7.4 Hz, 1H), 5.61 (dd, J=9.4, 3.5 Hz, 1H), 5.31 (dt, J=14.5, 7.1 Hz, 1H), 3.83 (dd, J=15.4, 3.4 Hz, 1H), 3.71 (d, J=5.6 Hz, 1H), 3.43 (d, J=7.1 Hz, 2H), 3.07 (dd, J=15.3, 9.4 Hz, 1H).

### 4-((3-oxo-2-((6-oxo-5-oxa-7-azaspiro[3.4]octan-2-yl)methyl)isoindolin-1-yl)methyl)pyridine-3,5-dicarbonitrile (459)

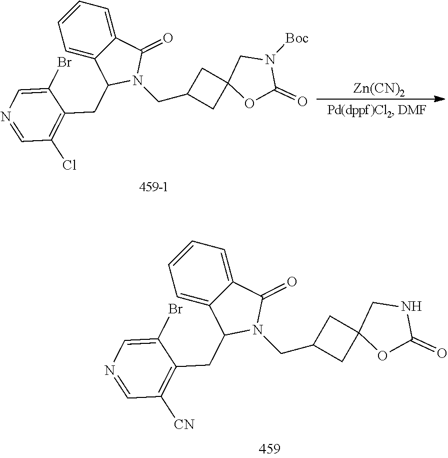

The intermediate 459-1 was prepared according to the procedure outlined for 432-1.

To a solution of tert-butyl 2-((1-((3-bromo-5-chloropyridin-4-yl)methyl)-3-oxoisoindolin-2-yl)methyl)-6-oxo-5-oxa-7-azaspiro[3.4]octane-7-carboxylate (280 mg, 0.49 mmol) in DMF (5 mL) were added Pd(dppf)Cl₂ (71 mg, 0.1 mmol) and Zn(CN)₂(124 mg, 1.07 mmol). The reaction mixture was stirred at 120° C. overnight. The reaction mixture was diluted with EA and washed with water. The organic layer was separated, dried over anhydrous Na₂SO₄ and concentrated under vacuum. The residue was purified by prep-HPLC to give 459 (86 mg, 42.6%) as a white solid. MS (ESI) m/z 414 [M+H]⁺. ¹H NMR (400 MHz, DMSO-d₆) δ 9.32 (s, 2H), 7.74-7.68 (m, 1H), 7.55-7.43 (m, 3H), 6.75-6.67 (m, 1H), 5.01 (dd, J=9.3, 4.9 Hz, 1H), 4.09-3.94 (m, 2H), 3.50 (s, 2H), 3.44 (dd, J=14.4, 5.3 Hz, 1H), 2.86 (dd, J=13.6, 9.4 Hz, 1H), 2.41-2.23 (m, 4H), 2.14-2.05 (m, 1H).

### 2-((1-((4-chloro-1-methyl-1H-pyrazol-5-yl)methyl)-4-methyl-3-oxoisoindolin-2-yl)methyl)-5-oxa-7-azaspiro[3.4]octan-6-one (460)

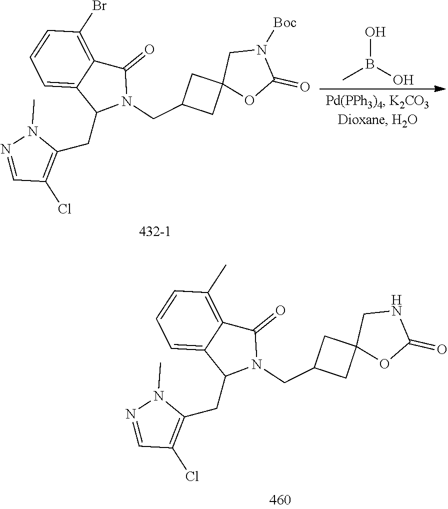

To a solution of 432-1 (150 mg, 0.26 mmol) and methylboronic acid (23 mg, 0.39 mmol) in 1,4-dioxane (2.4 mL) and water (0.6 mL) were added Pd(PPh₃)₄(30 mg, 0.026 mmol) and K₂CO₃ (72 mg, 0.52 mmol). The mixture was stirred at 120° C. overnight. The mixture was diluted with water and extracted with ethyl acetate. The combined organic phase was dried and concentrated. The residue was purified by Prep-HPLC to give 460 (21 mg, 19.6%) as a white solid. MS (ESI) m/z 415 [M+H]⁺. ¹H NMR (400 MHz, DMSO-d₆) δ 7.56 (s, 1H), 7.49 (s, 1H), 7.34 (t, J=7.6 Hz, 1H), 7.22 (d, J=7.5 Hz, 1H), 6.56 (d, J=7.5 Hz, 1H), 4.80 (dd, J=8.6, 5.7 Hz, 1H), 3.97 (dd, J=14.1, 6.0 Hz, 1H), 3.63 (s, 3H), 3.55-3.48 (m, 3H), 3.27 (dd, J=14.1, 6.2 Hz, 1H), 2.83 (dd, J=14.7, 8.7 Hz, 1H), 2.60 (s, 3H), 2.41-2.04 (m, 5H).

### 2-((3-((4-chloro-1-methyl-1H-pyrazol-5-yl)methyl)-5-(morpholinomethyl)-1-oxoisoindolin-2-yl)methyl)-5-oxa-7-azaspiro[3.4]octan-6-one (461)

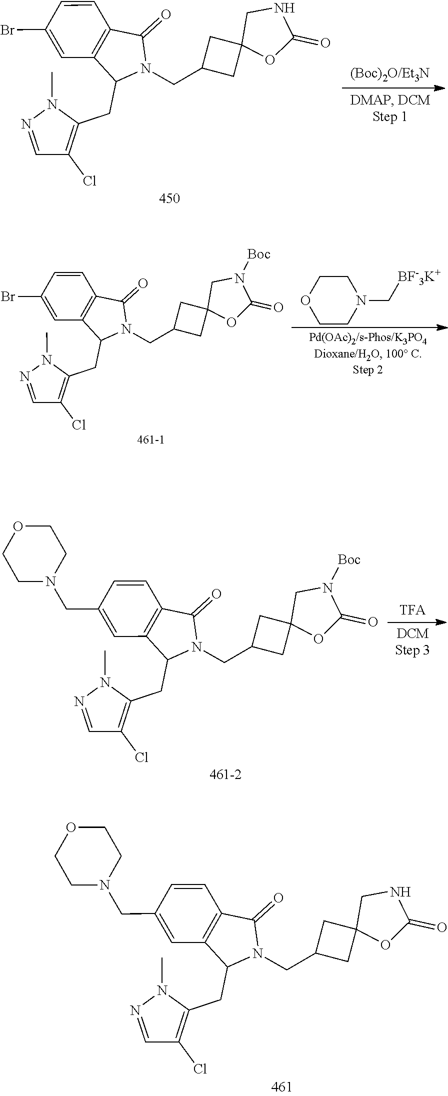

Step 1 was performed according to the procedure outlined for preparation of 432-1.

### Step 2. tert-butyl 2-((3-((4-chloro-1-methyl-1H-pyrazol-5-yl)methyl)-5-(morpholinomethyl)-1-oxoisoindolin-2-yl)methyl)-6-oxo-5-oxa-7-azaspiro[3.4]octane-7-carboxylate (461-2)

To a solution of 461-1 (100 mg, 0.17 mmol) in 1,4-dioxane (5 ml) and water (0.5 ml) were added potassium trifluoro(morpholinomethyl)boranuide (43 mg, 0.21 mmol), Pd(OAc)₂ (8 mg, 0.035 mmol), K₃PO₄ (73 mg, 0.35 mmol) and S-Phos (14 mg, 0.035 mmol). The reaction was stirred for 14 h at 100° C. and quenched with water. The mixture was extracted with EtOAc and washed with water. The organic layer was separated, dried over anhydrous Na₂SO₄ and concentrated under vacuum. The residue was purified by silica gel flash column chromatography (DCM/MeOH=10/1) to afford 461-2 (30 mg, 29.0%) as a yellow solid. MS (ESI) m/z 600 [M+H]⁺.

### Step 3. 2-((3-((4-chloro-1-methyl-1H-pyrazol-5-yl)methyl)-5-(morpholinomethyl)-1-oxoisoindolin-2-yl)methyl)-5-oxa-7-azaspiro[3.4]octan-6-one (461)

To a solution of 461-2 (30 mg, 0.05 mmol) in DCM (3 ml) was added 0.3 ml TFA. The mixture was stirred for 0.5 h at r.t. The product was purified by silica gel flash column chromatography (DCM/MeOH=10/1) to afford 461 (10 mg, 40%) as a white solid. MS (ESI) m/z 500 [M+H]⁺. ¹H NMR (400 MHz, CDCl₃) δ 7.81 (d, J=7.8 Hz, 1H), 7.51 (s, 1H), 7.48 (dd, J=7.8, 1.4 Hz, 11H), 6.65 (s, 1H), 5.35 (s, 11H), 4.84 (dd, J=9.1, 6.0 Hz, 11H), 4.20 (dd, J=14.4, 5.2 Hz, 11H), 3.75 (t, J=4.7 Hz, 4H), 3.61 (s, 2H), 3.59 (s, 2H), 3.52 (s, 3H), 3.42 (dd, J=14.5, 6.0 Hz, 1H), 3.14 (dd, J=14.4, 6.4 Hz, 1H), 2.69 (dd, J=14.5, 9.1 Hz, 11H), 2.53 (s, 4H), 2.46-2.27 (m, 5H).

### 2-((3-((4-chloro-1-methyl-1H-pyrazol-5-yl)methyl)-1-oxo-5-(pyrrolidin-1-ylmethyl)isoindolin-2-yl)methyl)-5-oxa-7-azaspiro[3.4]octan-6-one (462)

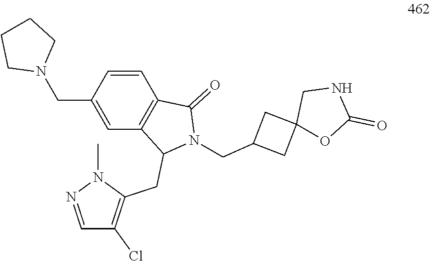 [SMILES: Cn1ncc(Cl)c1CC1c2cc(CN3CCCC3)ccc2C(=O)N1CC1CC2(CNC(=O)O2)C1]

The title compound 462 was prepared according to the procedure described for compound 461 as a white solid (6 mg, 7.5%, last 2 steps). MS (ESI) m/z 484 [M+H]⁺. ¹H NMR (400 MHz, CDCl₃) δ 7.83 (d, J=7.8 Hz, 1H), 7.54 (d, J=7.8 Hz, 1H), 7.49 (s, 11H), 6.83 (s, 1H), 5.21 (s, 1H), 4.85 (dd, J=8.3, 6.4 Hz, 1H), 4.19 (dd, J=14.4, 5.8 Hz, 11H), 3.98 (d, J=13.0 Hz, 11H), 3.84 (d, J=13.0 Hz, 1H), 3.60 (s, 2H), 3.54 (s, 3H), 3.36 (dd, J=14.7, 6.5 Hz, 1H), 3.08 (dd, J=14.3, 6.6 Hz, 1H), 2.92-2.83 (m, 4H), 2.78 (dd, J=14.6, 8.4 Hz, 1H), 2.44-2.24 (m, 5H), 1.98-1.91 (m, 4H).

### 2-((3-((4-chloro-1-methyl-1H-pyrazol-5-yl)methyl)-5-morpholino-1-oxoisoindolin-2-yl)methyl)-5-oxa-7-azaspiro[3.4]octan-6-one (463)

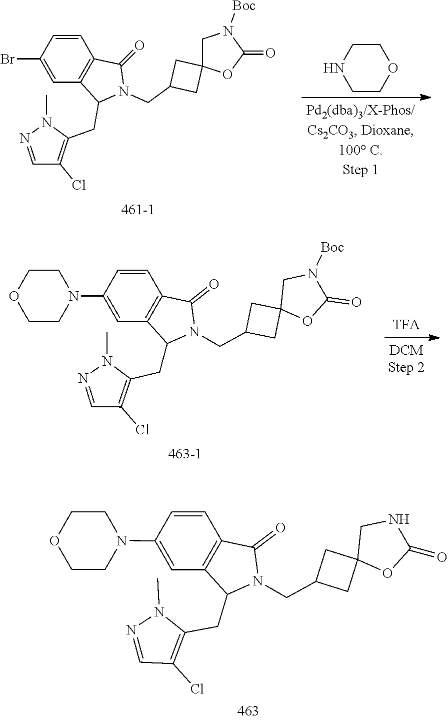 [SMILES: Cn1ncc(Cl)c1CC1c2cc(Br)ccc2C(=O)N1CC1CC2(C1)CN(C(=O)OC(C)(C)C)C(=O)O2 ; C1COCCN1 ; Cn1ncc(Cl)c1CC1c2cc(N3CCOCC3)ccc2C(=O)N1CC1CC2(C1)CN(C(=O)OC(C)(C)C)C(=O)O2 ; Cn1ncc(Cl)c1CC1c2cc(N3CCOCC3)ccc2C(=O)N1CC1CC2(CNC(=O)O2)C1]

### Step 1. tert-butyl 2-((3-((4-chloro-1-methyl-1H-pyrazol-5-yl)methyl)-5-morpholino-1-oxoisoindolin-2-yl)methyl)-6-oxo-5-oxa-7-azaspiro[3.4]octane-7-carboxylate (463-1)

To a solution of 461-1 (100 mg, 0.173 mmol) in 3 ml 1,4-dioxane were added morpholine (18 mg, 0.21 mmol), Pd₂(dba)₃ (16 mg, 0.017 mmol), Cs₂CO₃ (112 mg, 0.35 mmol) and X-Phos (16 mg, 0.035 mmol). The reaction mixture was stirred for 10 h at 100° C. and quenched with water. The mixture was extracted with EtOAc and washed with water. The organic layer was separated, dried over anhydrous Na₂SO₄ and concentrated under vacuum. The residue was purified by silica gel flash column chromatography (DCM/MeOH=20/1) to afford 463-1 as a yellow solid (98 mg, 97.0%). MS (ESI) m/z 586 [M+H]⁺.

Step 2 was performed according to the procedure described for step 3 for the preparation of 461 to afford 463 as a white solid (48 mg, 57.3%). MS (ESI) m/z 486 [M+H]⁺. ¹H NMR (400 MHz, CDCl₃) δ 7.70 (d, J=8.5 Hz, 1H), 7.51 (s, 11H), 6.94 (dd, J=8.5, 2.2 Hz, 1H), 5.85 (d, J=2.2 Hz, 1H), 5.55 (s, 1H), 4.73 (dd, J=9.8, 5.6 Hz, 1H), 4.16 (dd, J=14.3, 5.1 Hz, 1H), 3.80 (t, J=4.9 Hz, 4H), 3.61 (s, 2H), 3.53-3.48 (m, 1H), 3.47 (s, 3H), 3.15 (dd, J=14.3, 6.1 Hz, 1H), 3.12-3.01 (m, 4H), 2.55 (dd, J=14.3, 9.9 Hz, 1H), 2.47-2.28 (m, 5H).

### 3-((4-chloro-1-methyl-1H-pyrazol-5-yl)methyl)-1-oxo-2-((6-oxo-5-oxa-7-azaspiro[3.4]octan-2-yl)methyl)isoindoline-5-carbonitrile (464)

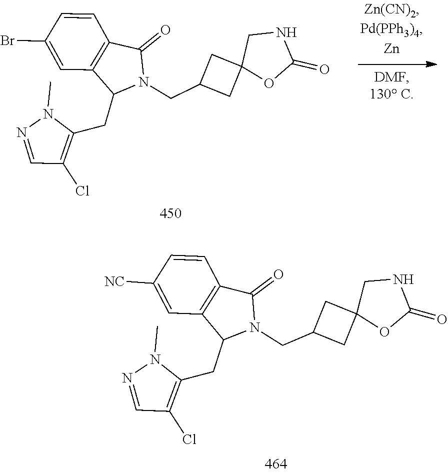 [SMILES: Cn1ncc(Cl)c1CC1c2cc(Br)ccc2C(=O)N1CC1CC2(CNC(=O)O2)C1 ; Cn1ncc(Cl)c1CC1c2cc(C#N)ccc2C(=O)N1CC1CC2(CNC(=O)O2)C1]

To a solution of 450 (100 mg, 0.21 mmol) in 3 ml DMF were added Zn(CN)₂ (73 mg, 0.63 mmol), Zn (14 mg, 0.21 mmol) and Pd(PPh₃)₄(73 mg, 0.042 mmol). The reaction mixture was stirred for 2 h at 130° C. using Biotage microwave reactor and quenched with ammonia water. The mixture was extracted with EtOAc and washed with water. The organic layer was separated, dried over anhydrous Na₂SO₄ and concentrated under vacuum. The residue was purified by silica gel flash column chromatography (DCM/MeOH=20/1) to afford 464 as a yellow solid (45 mg, 50.7%). MS (ESI) m/z 426 [M+H]⁺. ¹H NMR (400 MHz, CDCl₃) δ 7.95 (d, J=7.8 Hz, 1H), 7.78 (d, J=7.8 Hz, 1H), 7.53 (s, 1H), 6.94 (s, 1H), 5.86 (s, 1H), 4.91 (dd, J=9.3, 5.8 Hz, 1H), 4.19 (dd, J=14.3, 5.0 Hz, 1H), 3.60 (s, 2H), 3.58 (s, 3H), 3.46 (dd, J=14.8, 5.7 Hz, 1H), 3.21 (dd, J=14.5, 5.8 Hz, 1H), 2.71 (dd, J=14.6, 9.3 Hz, 1H), 2.48-2.27 (m, 5H).

### 2-((1-((4-chloro-1-ethyl-1H-pyrazol-5-yl)methyl)-3-oxoisoindolin-2-yl)methyl)-5-oxa-7-azaspiro[3.4]octan-6-one (465)

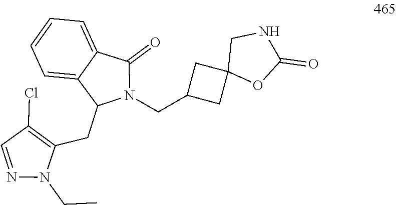 [SMILES: CCn1ncc(Cl)c1CC1c2ccccc2C(=O)N1CC1CC2(CNC(=O)O2)C1]

The title compound 465 was prepared according to the procedure described for compound 307 as a white solid (110 mg, 26%). MS (ESI) m/z 415 [M+H]⁺. ¹H NMR (400 MHz, CDCl₃) δ 7.86 (d, J=7.5 Hz, 1H), 7.56 (s, 1H), 7.50-7.44 (m, 1H), 7.41 (td, J=7.5, 1.2 Hz, 1H), 6.54 (d, J=7.5 Hz, 1H), 5.06 (s, 1H), 4.88 (dd, J=9.7, 5.6 Hz, 1H), 4.22 (dd, J=14.3, 5.0 Hz, 1H), 3.79 (dt, J=14.4, 7.2 Hz, 1H), 3.62 (m, 3H), 3.49 (dd, J=14.4, 5.7 Hz, 1H), 3.18 (dd, J=14.4, 6.3 Hz, 1H), 2.60 (dd, J=14.4, 9.8 Hz, 1H), 2.48-2.31 (m, 5H), 1.23 (t, J=7.2 Hz, 3H).

### trans-6-((1-((3-bromopyridin-2-yl)methyl)-3-oxoisoindolin-2-yl)methyl)hexahydrobenzo[d]oxazol-2(3H)-one (466)

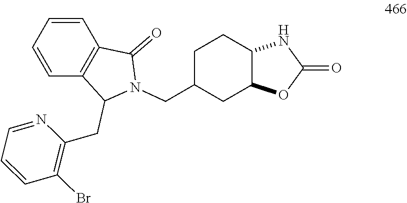

The title compound 466 was prepared according to the general procedure E as a white solid (6 mg, 18%). MS (ESI) m/z 456 [M+H]⁺. ¹H NMR (400 MHz, CD₃OD) δ 8.58-8.55 (m, 1H), 8.06-8.03 (m, 1H), 7.76-7.75 (m, 1H), 7.50-7.48 (m, 2H), 7.28-7.26 (m, 1H), 7.10-7.08 (m, 1H), 5.40-5.38 (m, 1H), 3.95-3.81 (m, 2H), 3.70-3.66 (m, 1H), 3.30-3.19 (m, 3H), 2.04-2.00 (m, 3H), 1.70-1.13 (m, 4H).

The intermediate 466-12 was prepared as follows (the stereochemistry in the structures of below scheme is relative configuration):

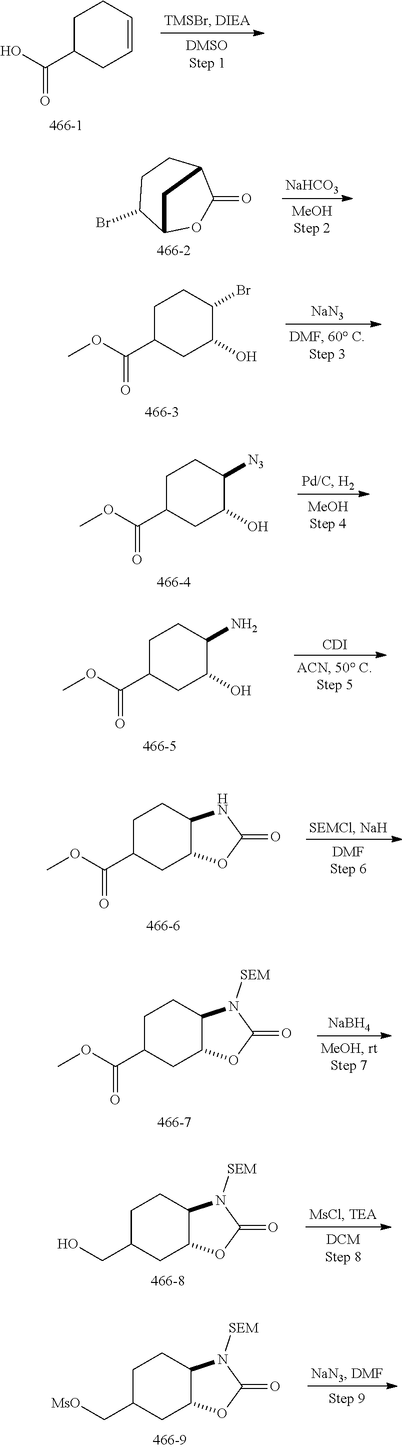

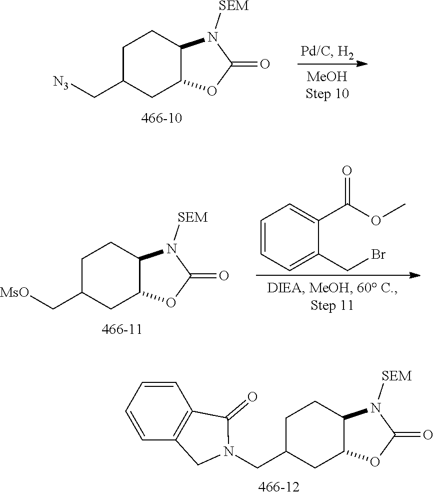

### Step 1. trans-4-bromo-6-oxabicyclo[3.2.1]octan-7-one (466-2)

To a solution of cyclohex-3-ene-1-carboxylic acid (40 g, 317 mmol) in DMSO (40 mL) were added TMSBr (58 g, 379 mmol) and DIEA (67 mL, 379 mmol) at 0° C. The reaction was stirred at r.t. overnight. The mixture was poured into water (200 mL) and extracted with EA (200 mL*2). The combined organic layers were washed with water (200 mL*2) and brine (200 mL), dried over anhydrous Na₂SO₄, filtered and concentrated. The residue was washed with hexane (30 mL) to give 466-2 (27 g, 42%) as a white solid.

### Step 2. methyl cis-4-bromo-3-hydroxycyclohexane-1-carboxylate (466-3)

A mixture of 466-2 (21 g, 107 mmol) and NaHCO₃ (10 g, 118 mmol) in MeOH (500 mL) was stirred overnight at r.t. The mixture was concentrated, added CHCl₃ (100 mL) and stirred at r.t. for 5 min; after filtration the filtrate was concentrated to give 466-3 (23 g, 91%) as a white solid.

¹H NMR (400 MHz, CD₃OD) δ 4.61-4.60 (m, 1H), 3.70 (s, 3H), 3.53-3.48 (m, 1H), 2.45-2.27 (m, 2H), 2.06-1.77 (m, 6H).

### Step 3. methyl trans-4-azido-3-hydroxycyclohexane-1-carboxylate (466-4)

To a solution of 466-3 (21 g, 89 mmol) in DMF (300 mL) was added NaN₃ (6 g, 98 mmol) at r.t. The mixture was stirred at 60′C overnight. To the mixture was added water (600 mL) and extracted with EA (300 mL*2). The combined organic layers were washed with water (200 mL*2) and brine (200 mL), dried over Na₂SO₄, filtered and concentrated under vacuum. The residue was purified by FCC (PE/EA=50/1˜5/1) to give 466-4 (11 g, 62%) as a yellow oil.

Step 4 was performed according to the procedure outlined for preparation of 181-4.

Step 5 was performed according to the procedure outlined for preparation of Int 1-2.

Step 6 was performed according to the procedure outlined for preparation of Int 1-3.

Step 7 was performed according to the procedure outlined for preparation of 164-2.

Step 8 was performed according to the procedure outlined for preparation of 132-2.

Step 9 was performed according to the procedure outlined for preparation of 466-4.

Step 10 was performed according to the procedure outlined for preparation of 181-4.

Step 11 was performed according to the procedure outlined for preparation of 403-2.

### 2-((1-((3-chloro-1-methyl-6-oxo-1,6-dihydropyridin-2-yl)methyl)-3-oxoisoindolin-2-yl)methyl)-5-oxa-7-azaspiro[3.4]octan-6-one (467)

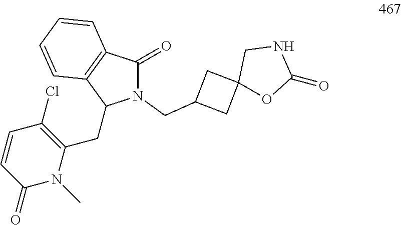 [SMILES: Cn1c(CC2c3ccccc3C(=O)N2CC2CC3(CNC(=O)O3)C2)c(Cl)ccc1=O]

The title compound 467 was prepared according to the procedure described for compound 451 using 381-8 as a reactant to afford a white solid (20 mg, 12%). MS (ESI) m/z 428 [M+H]⁺. ¹H NMR (400 MHz, CDCl₃) δ 7.89 (d, J=7.3 Hz, 1H), 7.55-7.43 (m, 2H), 7.41 (d, J=9.6 Hz, 1H), 6.76 (d, J=7.4 Hz, 1H), 6.64 (d, J=9.7 Hz, 1H), 5.06 (s, 1H), 4.99 (dd, J=9.4, 6.4 Hz, 1H), 4.21 (dd, J=15.1, 3.2 Hz, 1H), 3.70 (dd, J=14.1, 6.5 Hz, 1H), 3.61 (s, 2H), 3.19 (dd, J=13.9, 5.6 Hz, 1H), 3.10 (s, 3H), 2.81 (dd, J=14.0, 9.4 Hz, 1H), 2.47-2.34 (m, 5H).

### 2-((1-((4-chloro-1-methyl-1H-imidazol-5-yl)methyl)-3-oxoisoindolin-2-yl)methyl)-5-oxa-7-azaspiro[3.4]octan-6-one (468)

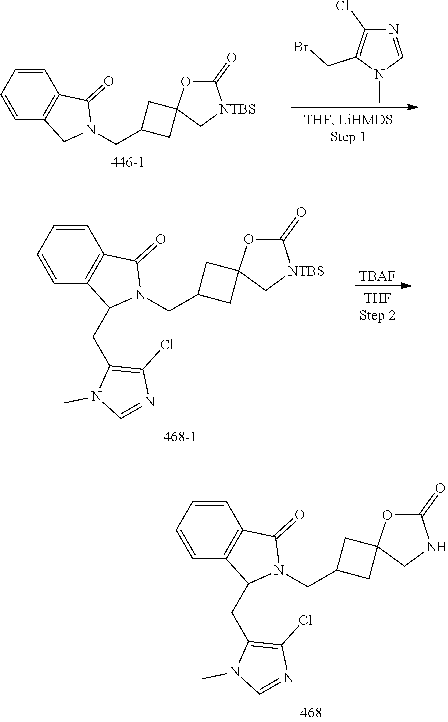

Step 1 was performed according to the procedure outlined for preparation of Int 12-2.

Step 2 was performed according to the procedure outlined for step 3 for preparation of compound 445 to afford 468 as a white solid (22 mg, 18%). MS (ESI) m/z 401 [M+H]⁺. ¹H NMR (400 MHz, CDCl₃) δ 7.86 (d, J=8.0 Hz, 1H), 7.52-7.38 (m, 2H), 7.36 (s, 1H), 6.62 (d, J=7.6 Hz, 1H), 5.04 (s, 1H), 4.82 (dd, J=9.7, 5.5 Hz, 1H), 4.23 (dd, J=14.4, 5.6 Hz, 1H), 3.62 (s, 2H), 3.46 (dd, J=14.8, 5.6 Hz, 1H), 3.23 (dd, J=14.4, 6.4 Hz, 1H), 3.20 (s, 3H), 2.54-2.33 (m, 6H).

### 2-((1-((6-chloroimidazo[2,1-b]thiazol-5-yl)methyl)-3-oxoisoindolin-2-yl)methyl)-5-oxa-7-azaspiro[3.4]octan-6-one (469)

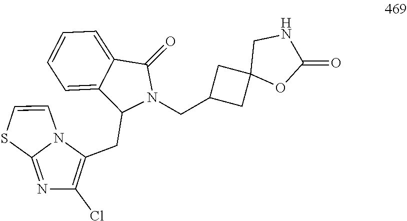 [SMILES: O=C1NCC2(CC(CN3C(=O)c4ccccc4C3Cc3c(Cl)nc4sccn34)C2)O1]

The title compound 469 was prepared according to the procedure described for compound 270 as a white solid (11 mg, 17.8%). MS (ESI) m/z 443 [M+H]⁺. ¹H NMR (400 MHz, DMSO-d₆) δ 7.71 (d, J=4.5 Hz, 1H), 7.58 (dd, J=6.7, 1.3 Hz, 1H), 7.51-7.37 (m, 3H), 7.28 (d, J=4.5 Hz, 1H), 7.19 (d, J=7.3 Hz, 1H), 4.93 (dd, J=6.6, 4.9 Hz, 1H), 3.99 (dd, J=14.0, 6.0 Hz, 1H), 3.68 (dd, J=15.4, 5.0 Hz, 1H), 3.49 (s, 2H), 3.42-3.29 (m, 2H), 2.37-2.25 (m, 1H), 2.28-2.13 (m, 3H), 2.13-2.05 (m, 1H).

### 2-((3-((4-chloro-1-methyl-1H-pyrazol-5-yl)methyl)-4-methyl-1-oxoisoindolin-2-yl)methyl)-5-oxa-7-azaspiro[3.4]octan-6-one (470)

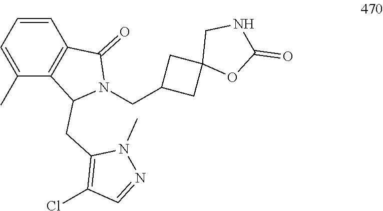 [SMILES: Cc1cccc2c1C(Cc1c(Cl)cnn1C)N(CC1CC3(CNC(=O)O3)C1)C2=O]

The title compound 470 was prepared according to the procedure described for compound 307 as a yellow solid (44 mg, 39%). MS (ESI) m/z 415 [M+H]⁺. ¹H NMR (400 MHz, CDCl₃) δ 7.71 (d, J=7.3 Hz, 1H), 7.48 (s, 1H), 7.42 (t, J=7.5 Hz, 1H), 7.36 (d, J=7.5 Hz, 1H), 5.05 (s, 1H), 4.92 (dd, J=8.1, 4.9 Hz, 1H), 4.15 (dd, J=14.2, 4.9 Hz, 1H), 3.56 (s, 3H), 3.55 (s, 2H), 3.26 (dd, J=15.0, 4.8 Hz, 1H), 2.88 (dd, J=15.0, 8.0 Hz, 1H), 2.54 (dd, J=14.2, 6.5 Hz, 1H), 2.27 (s, 3H), 2.33-2.13 (m, 5H).

### 2-((1-((5-chloro-1-cyclopropyl-1H-1,2,3-triazol-4-yl)methyl)-3-oxoisoindolin-2-yl)methyl)-5-oxa-7-azaspiro[3.4]octan-6-one (471)

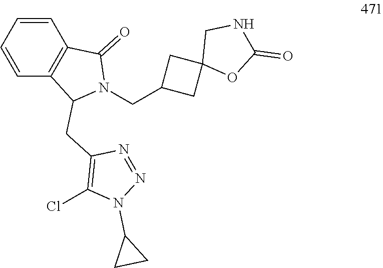 [SMILES: O=C1NCC2(CC(CN3C(=O)c4ccccc4C3Cc3nnn(C4CC4)c3Cl)C2)O1]

The title compound 471 was prepared according to the procedure described for compound 451 as a white solid (54 mg, 68%). MS (ESI) m/z 428 [M+H]⁺. ¹H NMR (400 MHz, CDCl₃) δ 7.72 (d, J=7.4 Hz, 1H), 7.49-7.36 (m, 2H), 7.20 (d, J=7.4 Hz, 1H), 5.98 (s, 1H), 4.84 (dd, J=6.5, 4.2 Hz, 1H), 4.15 (dd, J=14.5, 5.0 Hz, 1H), 3.87-3.82 (m, 1H), 3.60 (s, 2H), 3.34-3.20 (m, 2H), 3.05 (dd, J=15.0, 6.6 Hz, 1H), 2.43-2.32 (m, 5H), 1.26-1.16 (m, 2H), 1.14-0.96 (m, 2H).

The intermediate 471-3 was prepared as follows:

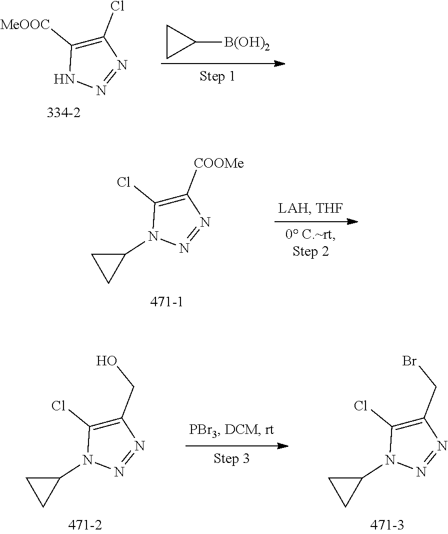

### Step 1: methyl 4-chloro-1-cyclopropyl-1H-1,2,3-triazole-5-carboxylate (471-1)

A mixture of 334-2 (3 g, 18 mmol), cyclopropylboronic acid (1.9 g, 22 mmol), dipiperidinomethane (8.2 g, 45 mmol) and Cu(AcO)₂ (162 mg, 1.8 mmol) in dried 1,4-dioxane (30 mL) was stirred at 83 T for 16 h. The mixture was filtered, and the filtrate was concentrated. The mixture was dissolved with ethyl acetate (30 mL), and the organic layer was washed with water (30 mL) and brine (30 mL), dried over Na₂SO₄, filtered, and concentrated in vacuo. The residue was purified by column chromatography on silica gel (eluting with PE/EA=3/1 to 2/1) to give 471-1 (320 mg, 8%) as a yellow oil. MS (ESI) m/z 202 [M+H]⁺.

Step 2 was performed according to the procedure outlined for preparation of 279-3.

Step 3 was performed according to the procedure outlined for preparation of 279-4.

### 2-((1-((5-chloro-1-methyl-6-oxo-1,6-dihydropyridin-2-yl)methyl)-3-oxoisoindolin-2-yl)methyl)-5-oxa-7-azaspiro[3.4]octan-6-one (472)

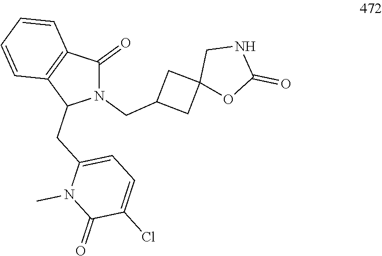 [SMILES: Cn1c(CC2c3ccccc3C(=O)N2CC2CC3(CNC(=O)O3)C2)ccc(Cl)c1=O]

The title compound 472 was prepared according to the procedure described for compound 467 as a white solid (4 mg, 2.4%). MS (ESI) m/z 428 [M+H]⁺. ¹H NMR (400 MHz, CDCl₃) δ 7.87 (d, J=7.0 Hz, 1H), 7.57-7.40 (m, 3H), 6.85 (d, J=7.4 Hz, 1H), 6.00 (d, J=7.5 Hz, 1H), 4.82 (s, 1H), 4.73 (dd, J=9.4, 5.3 Hz, 1H), 4.16 (d, J=15.3 Hz, 1H), 3.61 (s, 2H), 3.50 (s, 3H), 3.49 (dd, J=13.2, 6.6 Hz, 1H), 3.27 (dd, J=14.8, 5.6 Hz, 1H), 2.66 (dd, J=14.7, 9.4 Hz, 1H), 2.48-2.35 (m, 5H).

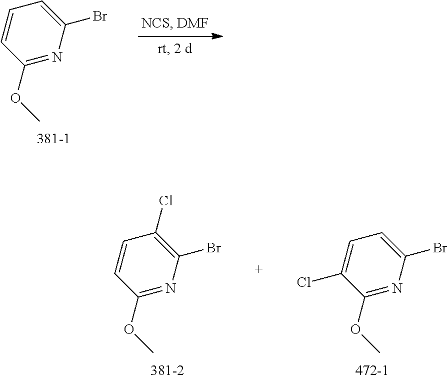

To a solution of 2-bromo-6-methoxypyridine (100 g, 0.53 mol) in DMF (300 mL) was added NCS (77.8 g, 0.58 mol) at r.t. The mixture was stirred for 2 d at 25° C. The mixture was allowed to cool down to r.t. The resulting mixture was diluted with water (1 L), and extracted with EtOAc (3*300 mL). The combined organic layers were washed with brine (300 mL), dried over anhydrous Na₂SO₄, and after filtration, the filtrate was concentrated under reduced pressure. The residue was purified by silica gel column chromatography eluting with PE/EA=1/1 to afford a crude product (84 g, white solid) containing 2 isomers: 2-bromo-3-chloro-6-methoxypyridine (80%) and 6-bromo-3-chloro-2-methoxypyridine (20%). MS (ESI) m/z 222 [M+H]⁺.

### 2-((3-((4-chloro-1-methyl-1H-pyrazol-5-yl)methyl)-5-(oxetan-3-yl)-1-oxoisoindolin-2-yl)methyl)-5-oxa-7-azaspiro[3.4]octan-6-one (473)

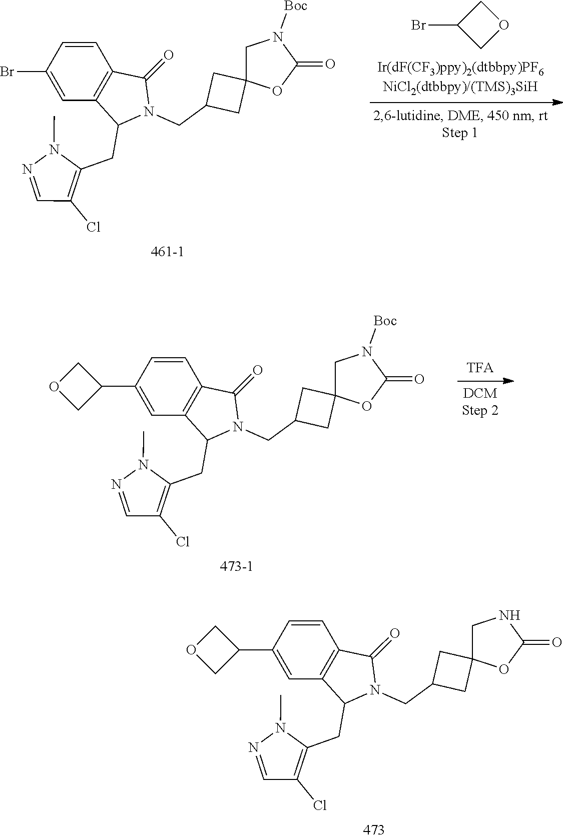 [SMILES: Cn1ncc(Cl)c1CC1c2cc(Br)ccc2C(=O)N1CC1CC2(C1)CN(C(=O)OC(C)(C)C)C(=O)O2 ; BrC1COC1 ; Cn1ncc(Cl)c1CC1c2cc(C3COC3)ccc2C(=O)N1CC1CC2(C1)CN(C(=O)OC(C)(C)C)C(=O)O2 ; Cn1ncc(Cl)c1CC1c2cc(C3COC3)ccc2C(=O)N1CC1CC2(CNC(=O)O2)C1]

### Step 1. tert-butyl 2-((3-((4-chloro-1-methyl-1H-pyrazol-5-yl)methyl)-5-(oxetan-3-yl)-1-oxoisoindolin-2-yl)methyl)-6-oxo-5-oxa-7-azaspiro[3.4]octane-7-carboxylate (473-1)

To a solution of 461-1 (120 mg, 0.21 mmol) in 3 mL DME were added 2,6-lutidine (45 mg, 0.42 mmol), 3-Bromooxetane (34 mg, 0.63 mmol), NiCl₂(dtbbpy) (4 mg, 0.01 mmol), (TMS)₃SiH (104 mg, 0.42 mmol) and Ir(dF(CF₃)ppy)₂(dtbbpy)PF((12 mg, 0.01 mmol). The mixture was stirred for 12 h at 450 nm lamp. The product was purified by silica gel flash column chromatography (DCM/MeOH=10/1) to give 473-1 (61 mg, 52.5%) as a white solid. MS (ESI) m/z 557 [M+H]⁺.

Step 2 was performed according to the procedure described for step 3 for the preparation of 461 to afford 473 as a white solid (10 mg, 40.0%). MS (ESI) m/z 457 [M+H]⁺. ¹H NMR (400 MHz, CDCl₃) δ 7.84 (d, J=7.8 Hz, 1H), 7.53 (s, 1H), 7.50-7.40 (m, 1H), 6.56 (s, 1H), 5.47 (s, 1H), 5.07-5.02 (m, 2H), 4.84 (dd, J=9.4, 5.9 Hz, 1H), 4.66-4.59 (m, 2H), 4.29-4.12 (m, 2H), 3.61 (s, 2H), 3.51-3.43 (m, 4H), 3.16 (dd, J=14.5, 6.2 Hz, 1H), 2.64 (dd, J=14.4, 9.4 Hz, 1H), 2.50-2.25 (m, 5H).

### 2-((5-chloro-3-((4-chloro-1-methyl-1H-pyrazol-5-yl)methyl)-1-oxoisoindolin-2-yl)methyl)-5-oxa-7-azaspiro[3.4]octan-6-one (474)

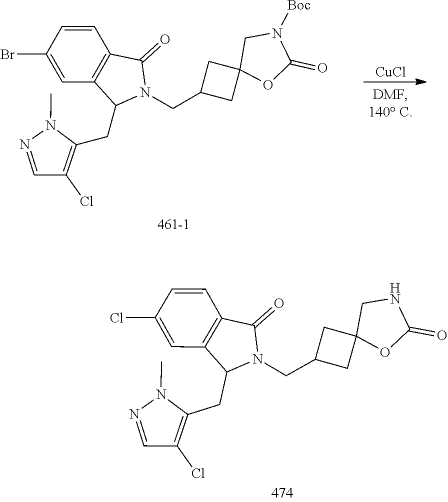

To a solution of 461-1 (100 mg, 0.17 mmol) in 3 mL DMF was added CuCl (68 mg, 0.69 mmol). The mixture was stirred for 12 h at 140° C. Water was added and extracted with EA (3 x). The combined organic phase was washed with brine, dried over Na₂SO₄, and concentrated in vacuo. The residue was purified by silica gel flash column chromatography (DCM/MeOH=20/1) to afford 474 as a white solid (30 mg, 40.0%). MS (ESI) m/z 435 [M+H]⁺. ¹H NMR (400 MHz, CDCl₃) δ 7.79 (d, J=8.0 Hz, 1H), 7.54 (s, 1H), 7.47 (dd, J=8.1, 1.7 Hz, 1H), 6.59 (d, J=1.7 Hz, 1H), 5.16 (s, 1H), 4.82 (dd, J=9.4, 5.7 Hz, 1H), 4.19 (dd, J=14.4, 5.1 Hz, 1H), 3.61 (s, 2H), 3.55 (s, 3H), 3.45 (dd, J=14.5, 5.7 Hz, 1H), 3.16 (dd, J=14.4, 6.3 Hz, 1H), 2.65 (dd, J=14.5, 9.4 Hz, 1H), 2.49-2.28 (m, 5H).

### 2-(2-(1H-pyrazol-4-yl)ethyl)-3-((3-bromopyridin-2-yl)methyl)isoindolin-1-one (475)

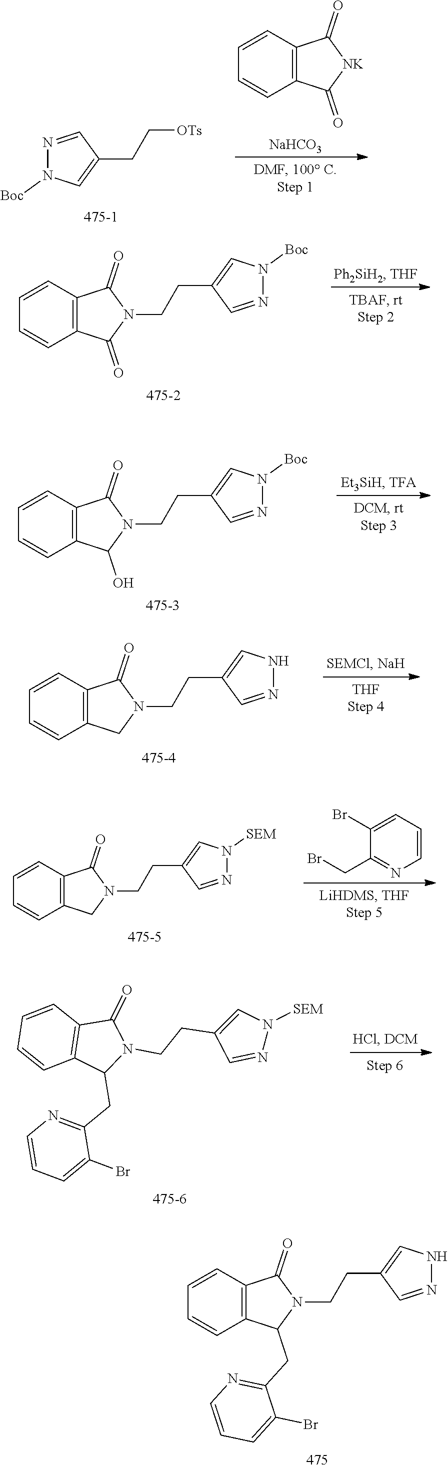

Step 1 was performed according to the procedure outlined for preparation of 15-1.

### Step 2. tert-butyl 4-(2-(1-hydroxy-3-oxoisoindolin-2-yl)ethyl)-1H-pyrazole-1-carboxylate (475-3)

To a solution of 475-2 (124 mg, 0.36 mmol) in dried THF (10 mL) were added Ph₂SiH₂ (135 mg, 0.72 mmol) and TBAF (2 mL) at 0° C., and the mixture was stirred at 50° C. for 3 h. The solvent was removed in vacuo to give crude product 475-3 (120 mg, 32%) as a white oil. MS (ESI) m/z 241 [M-Boc+H]⁺.

### Step 3. 2-(2-(1H-pyrazol-4-yl)ethyl)isoindolin-1-one (475-4)

To a solution of 475-3 (120 mg, 0.34 mmol) in dried DCM (5 mL) were added Et₃SiH (242 mg, 1.0 mmol) and TFA (3 mL). The mixture was stirred at 25° C. for 3 h. the mixture was washed with water (5 mL) and extracted with ethyl acetate (2*10 mL). The combined organic layer was dried over anhydrous Na₂SO₄ and concentrated. The residue was purified by column chromatography on silica gel (eluting with PE/EA=4/1 to 2/1) to give 475-4 (100 mg, 69%) as a white solid. MS (ESI) m/z 228 [M+H]⁺.

Step 4 was performed according to the procedure outlined for preparation of Int 4-2.

Step 5 was performed according to the procedure outlined for preparation of Int 12-2.

### Step 6. 2-((1H-benzo[d][1,2,3]triazol-5-yl)methyl)-3-((4-chloro-1-methyl-1H-1,2,3-triazol-5-yl)methyl)isoindolin-1-one (475)

To a solution of 475-6 (80 mg, 0.15 mmol) in DCM (2 mL) was added TFA (1 mL) at 0° C. The mixture was stirred for 3 h at r.t, and then concentrated. The residue was purified by Prep-HPLC to afford 475 (35 mg, 45%) as a white solid. MS (ESI) m/z 397 [M+H]⁺. ¹H NMR (400 MHz, CDCl₃) δ 8.59 (dd, J=4.7, 1.5 Hz, 1H), 7.92 (dd, J=8.1, 1.5 Hz, 1H), 7.86-7.82 (m, 1H), 7.47-7.40 (m, 4H), 7.18 (dd, J=8.0, 4.7 Hz, 1H), 7.06-7.00 (m, 1H), 5.31 (t, J=6.6 Hz, 1H), 4.27-4.14 (m, 1H), 3.53 (dd, J=15.1, 6.3 Hz, 1H), 3.36-3.19 (m, 2H), 2.93-2.72 (m, 2H).

### 2-((1-((3-bromopyridin-2-yl)methyl)-3-oxoisoindolin-2-yl)methyl)-5-oxa-7-azaspiro[3.4]octane-6,8-dione (476)

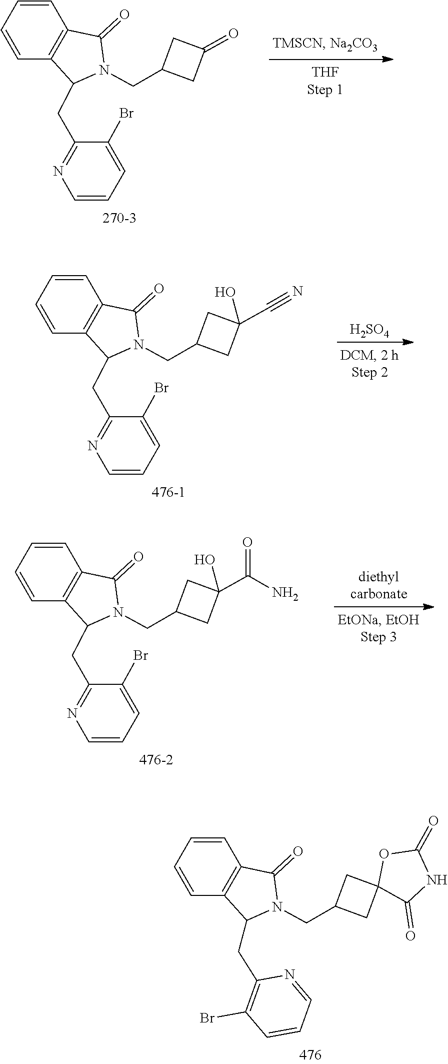

### Step 1: 3-((1-((3-bromopyridin-2-yl)methyl)-3-oxoisoindolin-2-yl)methyl)-1-hydroxycyclobutane-1-carbonitrile (476-1)

To a solution of 270-3 (600 mg, 1.56 mmol) and Na₂CO₃ (497 mg, 4.69 mmol) in THF (25 mL) was added TMSCN (170 mg, 1.72 mmol) in an ice-bath. The mixture was stirred at r.t. overnight. The mixture was diluted with EA (50 mL), washed with water (25 mL*2) and dried over anhydrous Na₂SO₄, filtered and concentrated. The residue was purified by column chromatography on silica gel (PE/EA=1/1) to give 476-1 (200 mg, 21%) as a white solid. MS (ESI) m/z 412 [M+H]⁺.

### Step 2: 3-((1-((3-bromopyridin-2-yl)methyl)-3-oxoisoindolin-2-yl)methyl)-1-hydroxycyclobutane-1-carboxamide (476-2)

To a solution of 476-1 (50 mg, 0.12 mmol) in DCM (5 mL) was added H₂SO₄ (238 mg, 2.43 mmol) at r.t. The reaction mixture was stirred at r.t. for 2 h. The mixture was diluted with water (10 mL), and basified with Na₂CO₃ to pH 8. After extraction with DCM (10 mL*2). The combined organic layers were washed with water (10 mL*2) and dried over anhydrous Na₂SO₄, filtered and concentrated. The residue was purified by prep-HPLC to give 476-2 (10 mg, 19%) as a white solid. MS (ESI) m/z 430 [M+H]⁺.

### Step 3: 2-[[1-[(3-bromo-2-pyridyl)methyl]-3-oxo-isoindolin-2-yl]methyl]-5-oxa-7-azaspiro[3.4]octane-6,8-dione (476)

To a solution of 476-2 (4 mg, 0.0093 mmol) in ethanol (0.1 mL) were added NaOEt (8 μL 210% NaOEt/EtOH, 0.019 mmol) and diethyl carbonate (6 μL, 0.047 mmol). The resulting mixture was refluxed under a N₂ atmosphere for 10 h, and then solvent was removed under vacuum. The residue was purified by a flash silica gel column (EtOAc/hexanes=1/1) to afford 476 (3 mg, 70.7%) as a white solid. MS (EST) m/z 456 [M+H]⁺. ¹H NMR (400 MHz, DMSO-d₆) δ 11.80 (s, 1H), 8.61-8.59 (m, 1H), 8.12-8.09 (m, 1H), 7.68-7.64 (m, 1H), 7.52-7.42 (m, 2H), 7.33-7.28 (m, 1H), 7.03-6.94 (m, 1H), 5.48-5.16 (m, 1H), 4.11-3.92 (m, 1H), 3.65 (dd, J=14.9, 5.4 Hz, 1H), 3.16 (dd, J=14.9, 7.7 Hz, 1H), 2.60-2.54 (m, 1H), 2.48-2.26 (m, 3H), 2.23-2.14 (m, 1H), 2.03-1.96 (m, 1H).

### 3-((4-chloro-1-methyl-1H-pyrazol-5-yl)methyl)-2-(3-(3-chloro-1H-pyrazol-4-yl)prop-2-yn-1-yl)isoindolin-1-one (477)

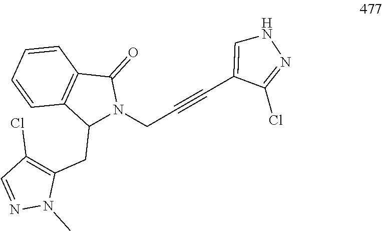 [SMILES: Cn1ncc(Cl)c1CC1c2ccccc2C(=O)N1CC#Cc1c[nH]nc1Cl]

The title compound 477 was prepared according to the procedure described for compound 452 as a white solid (3 mg, 28.7%). MS (ESI) m/z 441 [M+H]⁺. ¹H NMR (400 MHz, DMSO-d₆) δ 13.36 (s, 1H), 8.12 (s, 1H), 7.73 (dd, J=6.7, 1.6 Hz, 1H), 7.62-7.41 (m, 3H), 6.93 (d, J=7.3 Hz, 1H), 5.09 (dd, J=8.5, 5.8 Hz, 1H), 4.98 (d, J=18.1 Hz, 1H), 4.33 (d, J=18.0 Hz, 1H), 3.72 (s, 3H), 3.60 (dd, J=14.8, 5.8 Hz, 1H), 3.03 (dd, J=14.8, 8.6 Hz, 1H).

The intermediate 477-3 was prepared as follows:

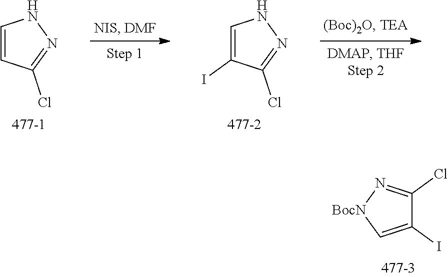 [SMILES: Clc1cc[nH]n1 ; Clc1n[nH]cc1I ; CC(C)(C)OC(=O)n1cc(I)c(Cl)n1]

### Step 1. 3-chloro-4-iodo-1H-pyrazole (477-2)

To a solution of 3-chloro-1H-pyrazole (500 mg, 4.88 mmol) in DMF (10 mL) was added NIS (1.43 g, 6.34 mmol). The mixture was stirred for 1 h at 25T. LC-MS showed the starting material was mostly converted to the product. Then the mixture was added water (10 mL) and extracted with EA (5 mL*3). The organics were then combined and dried (Na₂SO₄) before concentrated to dryness. The residue was purified by flash chromatography (PE/EtOAc=5/1) to give the product 477-2 (800 mg, 71.8%) as a yellow solid. MS (EST) m/z 229 [M+H]⁺.

### Step 2 was performed according to the procedure outlined for preparation of 23-2. 3-((4-chloro-1-methyl-1H-pyrazol-5-yl)methyl)-2-(3-(3-methyl-1H-pyrazol-4-yl)prop-2-yn-1-yl)isoindolin-1-one (478)

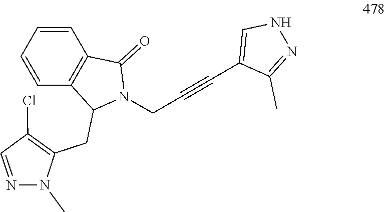 [SMILES: Cc1n[nH]cc1C#CCN1C(=O)c2ccccc2C1Cc1c(Cl)cnn1C]

The title compound 478 was prepared according to the procedure described for compound 477 as a white solid (3 mg, 26.4%). MS (ESI) m/z 380 [M+H]⁺. ¹H NMR (400 MHz, DMSO-d₆) δ 12.75 (s, 1H), 7.95-7.81 (m, 1H), 7.73 (d, J=6.6 Hz, 1H), 7.62-7.46 (m, 3H), 6.95 (d, J=7.3 Hz, 1H), 5.09 (dd, J=8.3, 6.0 Hz, 1H), 4.97 (d, J=18.0 Hz, 1H), 4.27 (d, J=18.0 Hz, 1H), 3.71 (s, 3H), 3.57 (dd, J=14.8, 6.0 Hz, 1H), 3.05 (dd, J=14.8, 8.4 Hz, 1H), 2.20 (s, 3H).

### 1,3-dimethyl-4-((3-oxo-2-((6-oxo-5-oxa-7-azaspiro[3.4]octan-2-yl)methyl)isoindolin-1-yl)methyl)-1H-pyrazole-5-carbonitrile (479)

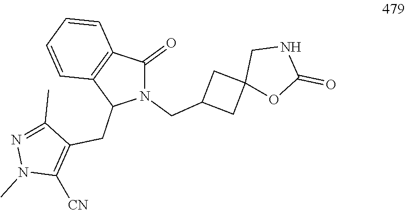 [SMILES: Cc1nn(C)c(C#N)c1CC1c2ccccc2C(=O)N1CC1CC2(CNC(=O)O2)C1]

The title compound 479 was prepared according to the procedure described for compound 270 as a white solid (160 mg, 80%). MS (ESI) m/z 406 [M+H]⁺. ¹H NMR (400 MHz, DMSO-d₆) δ 7.60 (d, J=7.2 Hz, 1H), 7.55-7.40 (m, 3H), 7.27 (d, J=7.6 Hz, 1H), 4.82 (dd, J=6.8, 4.4 Hz, 1H), 4.02 (dd, J=14.2, 6.0 Hz, 1H), 3.82 (s, 3H), 3.50 (s, 2H), 3.47-3.35 (m, 2H), 2.91 (dd, J=14.8, 6.9 Hz, 1H), 2.44-2.31 (m, 1H), 2.31-2.16 (m, 3H), 2.16-2.06 (m, 1H), 1.96 (s, 3H).

### 2-((1-((4-chloro-1-methyl-2-oxo-1,2-dihydropyridin-3-yl)methyl)-3-oxoisoindolin-2-yl)methyl)-5-oxa-7-azaspiro[3.4]octan-6-one (480)

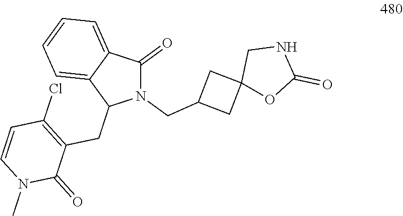 [SMILES: Cn1ccc(Cl)c(CC2c3ccccc3C(=O)N2CC2CC3(CNC(=O)O3)C2)c1=O]

The title compound 480 was prepared according to the procedure described for compound 270 as a white solid (42 mg, 5.9%). MS (ESI) m/z 428 [M+H]⁺. ¹H NMR (400 MHz, DMSO-d₆) δ 7.82 (d, J=7.3 Hz, 11H), 7.72-7.60 (m, 1H), 7.54-7.39 (m, 3H), 7.06-6.96 (m, 11H), 6.44 (d, J=7.3 Hz, 11H), 4.98 (dd, J=9.0, 5.3 Hz, 1H), 4.00 (dd, J=14.1, 5.5 Hz, 1H), 3.54-3.46 (m, 5H), 3.39-3.27 (m, 2H), 2.63 (dd, J=12.9, 9.1 Hz, 1H), 2.41-2.31 (m, 1H), 2.30-2.19 (m, 3H), 2.11-2.01 (m, 11H).

The intermediate 480-6 was prepared as follows:

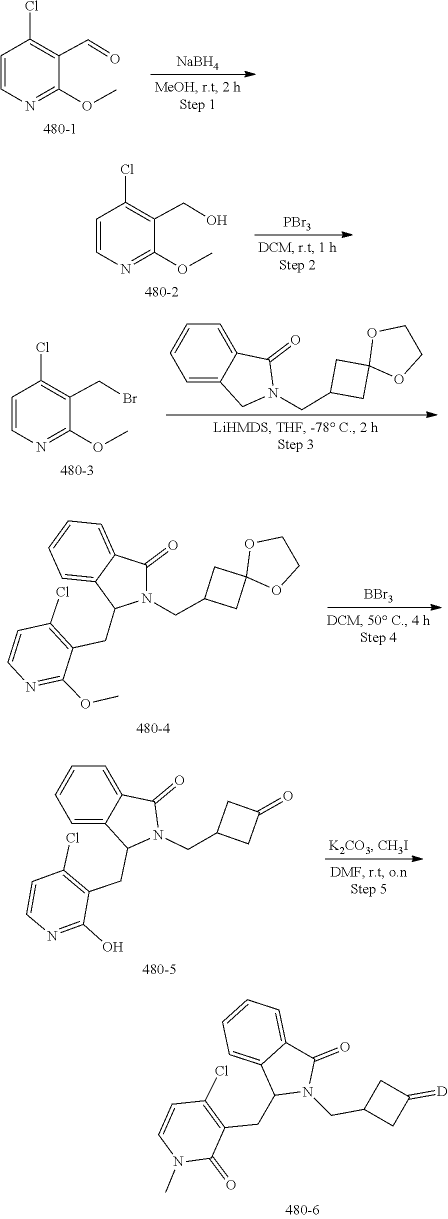

Step 1 was performed according to the procedure outlined for preparation of 333-6.

Step 2 was performed according to the procedure outlined for preparation of 263-3.

Step 3 was performed according to the procedure outlined for preparation of Int 12-2.

### Step 4. 3-((4-chloro-2-hydroxypyridin-3-yl)methyl)-2-((3-oxocyclobutyl)methyl) isoindolin-1-one (480-5)

To a stirred solution of 480-4 (608 mg, 1.46 mmol) in DCM (10 mL) was added BBr₃ (1M, 2.2 mL, 2.20 mmol) dropwise at 0° C. The mixture was stirred at 50° C. for 4 h. The reaction mixture was adjusted to pH at about 7-8 using sat. NaHCO₃ and extracted with DCM (3*20 mL). The combined organic phase was washed with brine, dried over anhydrous Na₂SO₄ and concentrated to afford 480-5 (500 mg, crude) as a brown oil. MS (ESI) m/z 357 [M+H]⁺.

Step 5 was performed according to the procedure outlined for preparation of 315-3.

### 2-((1-((1,4-dimethyl-2-oxo-1,2-dihydropyridin-3-yl)methyl)-3-oxoisoindolin-2-yl)methyl)-5-oxa-7-azaspiro[3.4]octan-6-one (481)

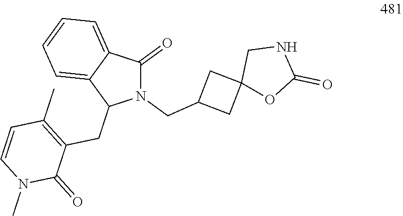 [SMILES: Cc1ccn(C)c(=O)c1CC1c2ccccc2C(=O)N1CC1CC2(CNC(=O)O2)C1]

The title compound 481 was prepared according to the procedure described for compound 270 as a white solid (51 mg, 8.6%). MS (ESI) m/z 408 [M+H]⁺. ¹H NMR (400 MHz, DMSO-d₆) δ 7.73-7.65 (m, 1H), 7.63 (d, J=6.9 Hz, 1H), 7.52-7.40 (m, 3H), 6.94-6.86 (m, 1H), 6.14 (d, J=6.9 Hz, 1H), 4.97 (dd, J=9.3, 5.2 Hz, 1H), 4.00 (dd, J=13.9, 5.6 Hz, 11H), 3.56-3.46 (m, 5H), 3.33-3.24 (m, 2H), 2.43-2.32 (m, 2H), 2.33-2.22 (m, 3H), 2.11-2.01 (m, 1H), 1.81 (s, 3H).

The intermediate 481-6 was prepared as follows:

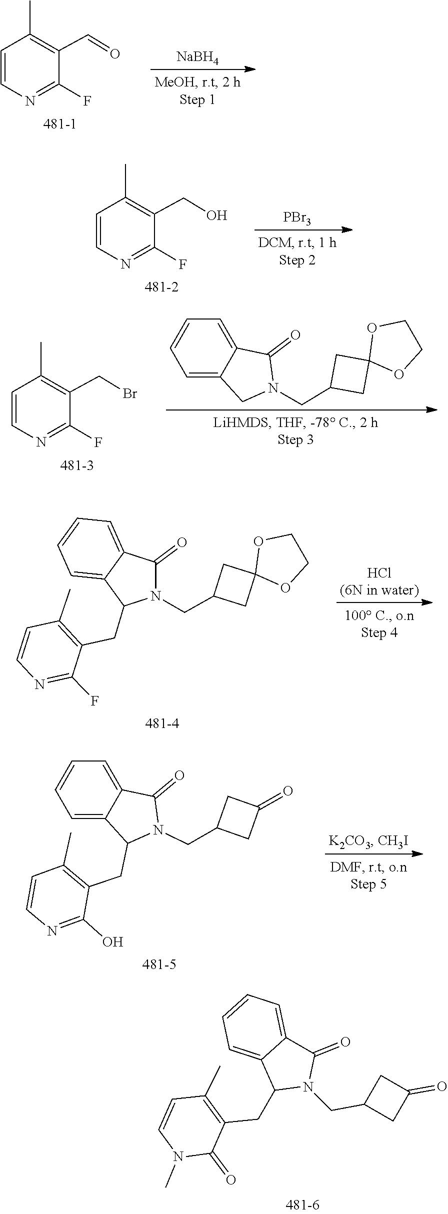

Step 1 was performed according to the procedure outlined for preparation of 333-6.

Step 2 was performed according to the procedure outlined for preparation of 195-2.

Step 3 was performed according to the procedure outlined for preparation of Int 12-2.

### Step 4. 3-((2-hydroxy-4-methylpyridin-3-yl)methyl)-2-((3-oxocyclobutyl)methyl) isoindolin-1-one (481-5)

A mixture of 481-4 (650 mg, 1.70 mmol) in HCl (6N in water, 18 mL) was stirred at 100° C. overnight. The reaction mixture was cooled to r.t. The precipitated solid was collected by filtration and washed with water. The collected solid was dried in an oven to afford 481-5 (497 mg, 86.9%) as a gray solid. MS (ESI) m/z 337 [M+H]⁺.

Step 5 was performed according to the procedure outlined for preparation of 315-3.

### 2-((1-((4-chloro-1-methyl-1H-pyrazol-5-yl)methyl)-3-thioxoisoindolin-2-yl)methyl)-5-oxa-7-azaspiro[3.4]octan-6-one (482)

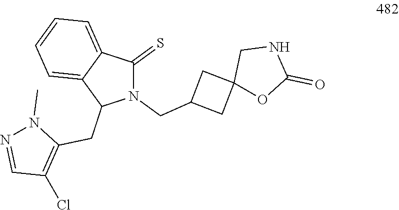 [SMILES: Cn1ncc(Cl)c1CC1c2ccccc2C(=S)N1CC1CC2(CNC(=O)O2)C1]

The title compound 482 was prepared according to the procedure described for compound 270 as a white solid (12 mg, 7.5%). MS (ESI) m/z 417 [M+H]⁺. ¹H NMR (400 MHz, DMSO-d₆) δ 7.88-7.81 (m, 1H), 7.56-7.46 (m, 4H), 6.79-6.72 (m, 1H), 5.20 (dd, J=8.8, 5.5 Hz, 1H), 4.75 (dd, J=13.1, 3.9 Hz, 1H), 3.87-3.70 (m, 2H), 3.64 (s, 3H), 3.51 (s, 2H), 2.96 (dd, J=14.8, 8.9 Hz, 1H), 2.46-2.18 (m, 5H).

The intermediate 482-1 was prepared as follows:

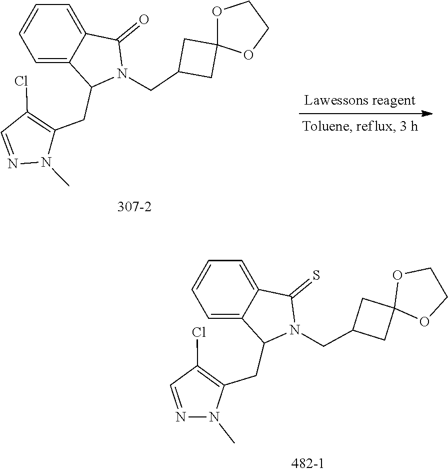 [SMILES: Cn1ncc(Cl)c1CC1c2ccccc2C(=O)N1CC1CC2(C1)OCCO2 ; Cn1ncc(Cl)c1CC1c2ccccc2C(=S)N1CC1CC2(C1)OCCO2]

To a solution of 307-2 (230 mg, 0.59 mmol) in Toluene (10 mL) was added Lawessons reagent (240 mg, 0.59 mmol). The mixture was stirred at 110° C. for 3 h. The reaction mixture was concentrated under vacuum. The residue was purified by a silica gel column, and eluted with PE/EA=3/1 to afford 2-((5,8-dioxaspiro[3.4]octan-2-yl)methyl)-3-((4-chloro-1-methyl-iH-pyrazol-5-yl)methyl)isoindoline-1-thione (174 mg, 72.5%) as a brown oil. MS (ESI) m/z 404 [M+H]⁺.

### (2r,4r)-2-((1-((4-chloro-1-methyl-1H-pyrazol-5-yl)methyl)-3-oxoisoindolin-2-yl)methyl)-5-oxa-7-azaspiro[3.4]octan-6-one (483)

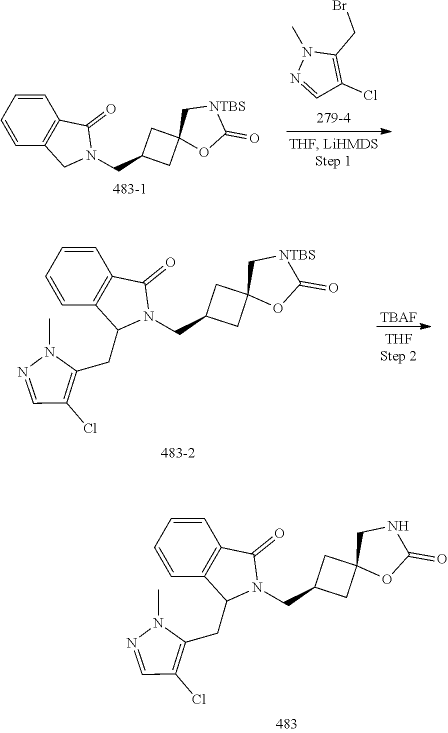

483-1 was obtained when purification of 446-1 by flash silica gel column chromatography eluting with PE/EA=1/1 was conducted.

Step 1 was performed according to the procedure outlined for preparation of Int 12-2.

Step 2 was performed according to the procedure outlined for step 3 for preparation of compound 445 to afford 483 as a white solid (35 mg, 22%). MS (ESI) m/z 401 [M+H]⁺. ¹H NMR (400 MHz, CDCl₃) δ 7.86 (d, J=7.2 Hz, 1H), 7.52 (s, 1H), 7.51-7.39 (m, 2H), 6.57 (d, J=7.6 Hz, 1H), 5.04 (s, 1H), 4.83 (dd, J=9.7, 5.6 Hz, 1H), 4.13 (dd, J=14.4, 9.7 Hz, 1H), 3.76-3.65 (m, 2H), 3.48 (s, 3H), 3.48-3.41 (m, 1H), 3.28 (dd, J=14.3, 5.6 Hz, 1H), 2.86-2.71 (m, 2H), 2.66-2.50 (m, 2H), 2.23-2.03 (m, 2H).

### 2-((1-((7-chloro-2-methyl-1,2,3,4-tetrahydropyrrolo[1,2-a]pyrazin-6-yl)methyl)-3-oxoisoindolin-2-yl)methyl)-5-oxa-7-azaspiro[3.4]octan-6-one (484)

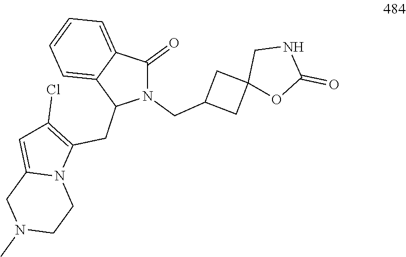 [SMILES: CN1CCn2c(cc(Cl)c2CC2c3ccccc3C(=O)N2CC2CC3(CNC(=O)O3)C2)C1]

The title compound 484 was prepared according to the procedure described for compound 468 as a white solid (2 mg, 6.2%). MS (ESI) m/z 455 [M+H]⁺. ¹H NMR (400 MHz, CD₃OD) δ 7.71-7.62 (m, 1H), 7.44-7.35 (m, 2H), 6.70-6.63 (m, 1H), 5.82 (s, 1H), 4.81 (dd, J=9.1, 6.3 Hz, 1H), 4.02 (dd, J=14.2, 6.1 Hz, 1H), 3.69-3.58 (m, 1H), 3.57-3.43 (m, 4H), 3.37-3.24 (m, 3H), 2.75-2.65 (m, 1H), 2.64-2.53 (m, 2H), 2.41-2.35 (m, 1H), 2.33 (s, 3H), 2.30-2.23 (m, 2H), 2.23-2.10 (m, 2H).

The intermediate 484-6 was prepared as follows:

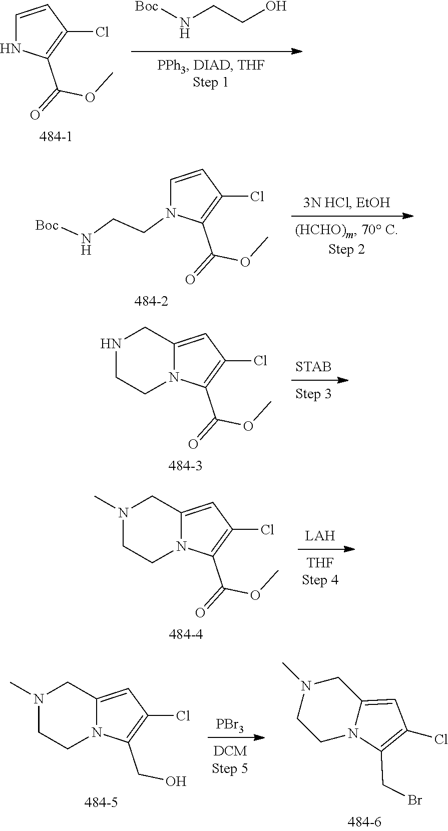

### Step 1. methyl 1-(2-((tert-butoxycarbonyl)amino)ethyl)-3-chloro-1H-pyrrole-2-carboxylate (484-2)

To a solution of methyl 3-chloro-JH-pyrrole-2-carboxylate (2 g, 12.5 mmol), Boc-Glycinol (2.63 g, 16.3 mmol) and PPh₃ (4.27 g, 16.3 mmol) in THF (60 mL) was added DIAD (3.29 g, 16.3 mmol) under N₂. The mixture was stirred for 2 h. The organic layer was concentrated under vacuum. The residue was purified by silica gel column chromatography (PE/EtOAc=3/1) to give 484-2 (3.5 g, 92%) as a yellow oil. MS (ESI) m/z 303 [M+H]⁺.

### Step 2. methyl 7-chloro-1,2,3,4-tetrahydropyrrolo[1,2-a]pyrazine-6-carboxylate (484-3)

To a solution of 484-2 (2.5 g, 8.26 mmol) in methanol (25 mL) was added 3N HCl (8 mL). The mixture was stirred for 3 h. Formaldehyde (743 mg, 24.8 mmol) was added and the reaction was stirred for 14 h at 80° C. The reaction mixture was concentrated under vacuum. The residue was purified by C18 column to give 484-3 (1.2 g, 67%) as a yellow solid. MS (ESI) m/z 215 [M+H]⁺.

### Step 3. methyl 7-chloro-2-methyl-1,2,3,4-tetrahydropyrrolo[1,2-a]pyrazine-6-carboxylate (484-4)

To a solution of 484-3 (1.2 g, 5.59 mmol) in methanol (15 mL) were added formaldehyde (335 mg, 11.2 mmol) and STAB (1.3 g, 6.15 mmol). The mixture was stirred for 14 h. The mixture was concentrated under vacuum. The residue was purified by C18 column to give 484-4 (558 mg, 43%) as a yellow solid. MS (ESI) m/z 229 [M+H]⁺.

Step 4 was performed according to the procedure outlined for preparation of 263-2.

Step 5 was performed according to the procedure outlined for preparation of 263-3.

### 2-((3-((4-chloro-1-methyl-1H-pyrazol-5-yl)methyl)-5-methyl-1-oxoisoindolin-2-yl)methyl)-5-oxa-7-azaspiro[3.4]octan-6-one (485)

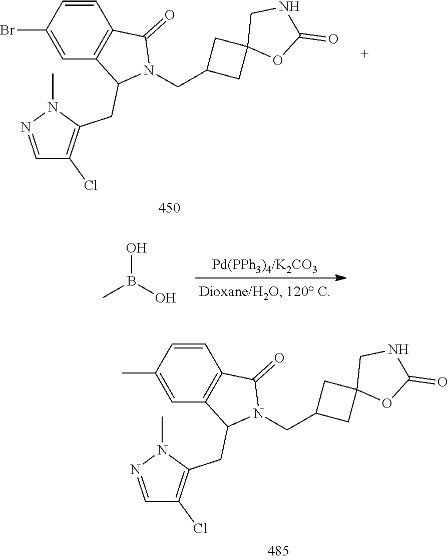

The title compound 485 was prepared according to the procedure described for compound 460 as a white solid (20 mg, 28.0%). MS (ESI) m/z 415 [M+H]⁺. ¹H NMR (400 MHz, CDCl₃) δ 7.72 (d, J=7.7 Hz, 1H), 7.52 (s, 1H), 7.27 (d, J=7.1 Hz, 1H), 6.35 (s, 1H), 5.61 (s, 1H), 4.78 (dd, J=9.4, 5.7 Hz, 1H), 4.18 (dd, J=14.4, 5.1 Hz, 1H), 3.60 (s, 2H), 3.47 (s, 3H), 3.45-3.39 (m, 1H), 3.13 (dd, J=14.5, 6.3 Hz, 1H), 2.60 (dd, J=14.4, 9.5 Hz, 1H), 2.46-2.27 (m, 8H).

### 2-((3-((4-chloro-1-methyl-1H-pyrazol-5-yl)methyl)-5-fluoro-1-oxoisoindolin-2-yl)methyl)-5-oxa-7-azaspiro[3.4]octan-6-one (486)

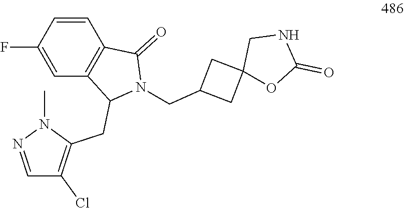 [SMILES: Cn1ncc(Cl)c1CC1c2cc(F)ccc2C(=O)N1CC1CC2(CNC(=O)O2)C1]

The title compound 486 was prepared according to the procedure described for compound 307 as a white solid (120 mg, 77.5%). MS (ESI) m/z 419 [M+H]⁺. ¹H NMR (400 MHz, CDCl₃) δ 7.82 (dd, J=8.4, 5.0 Hz, 1H), 7.51 (s, 1H), 7.16 (td, J=8.7, 2.3 Hz, 1H), 6.26 (dd, J=8.2, 2.2 Hz, 1H), 5.93 (s, 1H), 4.81 (dd, J=9.6, 5.7 Hz, 1H), 4.16 (dd, J=14.5, 5.3 Hz, 1H), 3.60 (s, 2H), 3.54 (s, 3H), 3.45 (dd, J=14.5, 5.7 Hz, 1H), 3.17 (dd, J=14.5, 6.4 Hz, 1H), 2.64 (dd, J=14.5, 9.6 Hz, 1H), 2.50-2.25 (m, 5H).

### (2r,4r)-2-((1-((4-chloro-1-methyl-1H-pyrazol-5-yl)methyl)-3-oxoisoindolin-2-yl)methyl)-2-fluoro-5-oxa-7-azaspiro[3.4]octan-6-one (487) and (2s,4s)-2-((1-((4-chloro-1-methyl-1H-pyrazol-5-yl)methyl)-3-oxoisoindolin-2-yl)methyl)-2-fluoro-5-oxa-7-azaspiro[3.4]octan-6-one (507)

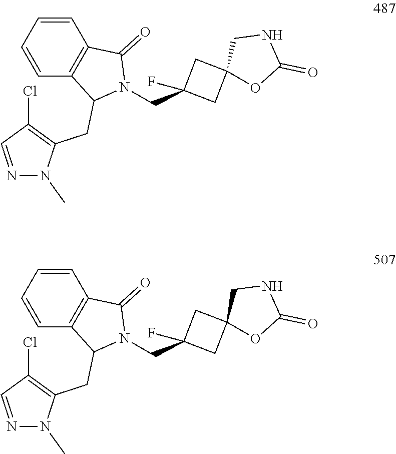 [SMILES: Cn1ncc(Cl)c1CC1c2ccccc2C(=O)N1C[C@]1(F)C[C@@]2(CNC(=O)O2)C1 ; Cn1ncc(Cl)c1CC1c2ccccc2C(=O)N1C[C@]1(F)C[C@]2(CNC(=O)O2)C1]

The title compounds 487 and 507 were prepared according to the procedure described for compound 468. Compound 487 was a yellow solid. MS (ESI) m/z 419 [M+H]⁺. ¹H NMR (400 MHz, CDCl₃) δ 7.89 (dt, J=7.5, 1.1 Hz, 1H), 7.60-7.37 (m, 3H), 6.51 (dd, J=7.6, 1.0 Hz, 1H), 5.15 (s, 1H), 5.02 (dd, J=10.2, 5.4 Hz, 1H), 4.58 (dd, J=34.3, 15.3 Hz, 1H), 3.73 (q, J=9.2 Hz, 2H), 3.65-3.53 (m, 2H), 3.50 (s, 3H), 3.03-2.87 (m, 2H), 2.73-2.59 (m, 1H), 2.59-2.40 (m, 2H). Compound 507 was a yellow solid. MS (ESI) m/z 419 [M+H]⁺. ¹H NMR (400 MHz, CDCl₃) δ 7.89 (dt, J=7.5, 1.1 Hz, 1H), 7.60-7.37 (m, 3H), 6.51 (dd, J=7.6, 1.0 Hz, 1H), 5.15 (s, 1H), 5.02 (dd, J=10.2, 5.4 Hz, 1H), 4.58 (dd, J=34.3, 15.3 Hz, 1H), 3.73 (q, J=9.2 Hz, 2H), 3.65-3.53 (m, 2H), 3.50 (s, 3H), 3.03-2.87 (m, 2H), 2.73-2.59 (m, 1H), 2.59-2.40 (m, 2H).

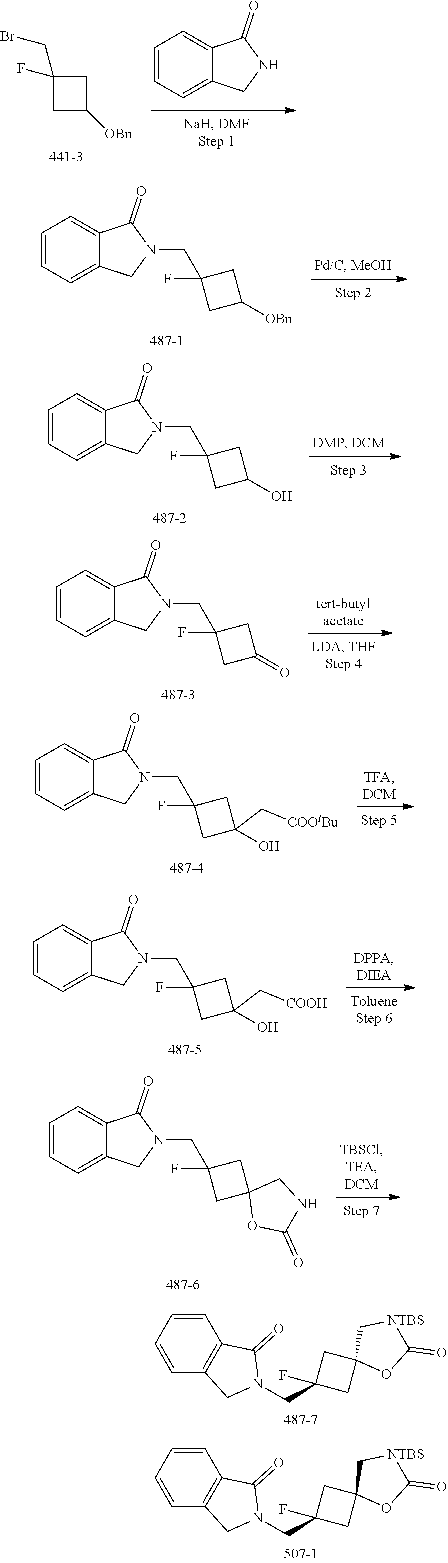

The intermediate 487-3 was prepared according to the procedure described for 441-6. The intermediates 487-7 and 507-1 were prepared according to the procedure described for 445-1. In step 7, two isomers (487-7 and 507-1) were obtained after purification.

### 2-((1-((2-chloroimidazo[1,2-a]pyridin-3-yl)methyl)-3-oxoisoindolin-2-yl)methyl)-5-oxa-7-azaspiro[3.4]octan-6-one (488)

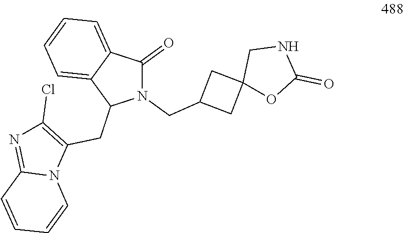 [SMILES: O=C1NCC2(CC(CN3C(=O)c4ccccc4C3Cc3c(Cl)nc4ccccn34)C2)O1]

The title compound 488 was prepared according to the procedure described for compound 270 as a white solid (11 mg, 22.1%). MS (ESI) m/z 437 [M+H]⁺. ¹H NMR (400 MHz, DMSO-d₆) δ 8.36 (d, J=6.9 Hz, 1H), 7.60 (d, J=6.7 Hz, 1H), 7.55-7.46 (m, 2H), 7.44-7.26 (m, 2H), 6.95 (td, J=6.9, 1.2 Hz, 1H), 6.86 (d, J=7.2 Hz, 1H), 4.98 (dd, J=7.5, 5.6 Hz, 1H), 4.00 (dd, J=14.0, 5.7 Hz, 1H), 3.81 (dd, J=15.3, 5.6 Hz, 1H), 3.48 (s, 2H), 3.42-3.26 (m, 2H), 2.36-2.04 (m, 5H).

### 2-((1-((4-chloro-1-methyl-1H-1,2,3-triazol-5-yl)methyl)-3-oxoisoindolin-2-yl)methyl)-5-oxa-7-azaspiro[3.4]octan-6-one (489)

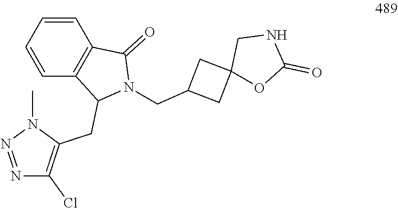 [SMILES: Cn1nnc(Cl)c1CC1c2ccccc2C(=O)N1CC1CC2(CNC(=O)O2)C1]

The title compound 489 was prepared according to the procedure described for compound 270 using 334-5 as a starting material to afford a white solid (99 mg, 33%). MS (EST) m/z 402 [M+H]+¹H NMR (400 MHz, DMSO-d₆) δ 7.65 (d, J=7.2 Hz, 1H), 7.56-7.45 (m, 2H), 7.02 (d, J=7.6 Hz, 1H), 4.95 (dd, J=7.6, 0.8 Hz, 1H), 4.00 (dd, J=14.0, 6.4 Hz, 1H), 3.87 (s, 3H), 3.64 (dd, J=15.2, 5.2 Hz, 11H), 3.50 (s, 2H), 3.41 (dd, J=14.4, 6.8 Hz, 11H), 3.07 (dd, J=15.2, 7.6 Hz, 1H), 2.45-2.04 (m, 5H).

### 3-((4-chloro-1-methyl-1H-pyrazol-5-yl)methyl)-2-((6-thioxo-5-oxa-7-azaspiro[3.4]octan-2-yl)methyl)isoindolin-1-one (490)

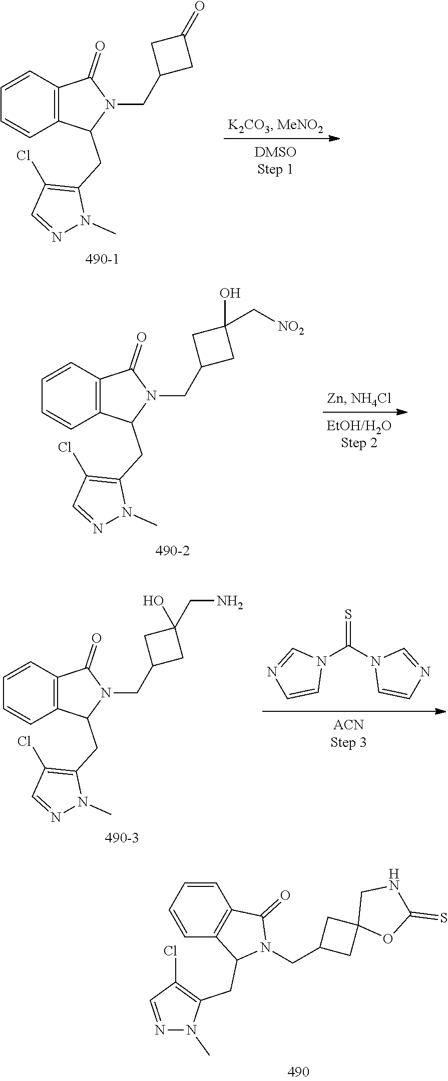 [SMILES: Cn1ncc(Cl)c1CC1c2ccccc2C(=O)N1CC1CC(=O)C1 ; Cn1ncc(Cl)c1CC1c2ccccc2C(=O)N1CC1CC(O)(C[N+](=O)[O-])C1 ; Cn1ncc(Cl)c1CC1c2ccccc2C(=O)N1CC1CC(O)(CN)C1 ; S=C(n1ccnc1)n1ccnc1 ; Cn1ncc(Cl)c1CC1c2ccccc2C(=O)N1CC1CC2(CNC(=S)O2)C1]

### Step 1. 3-((4-chloro-1-methyl-1H-pyrazol-5-yl)methyl)-2-((3-hydroxy-3-(nitromethyl)cyclobutyl)methyl)isoindolin-1-one (490-2)

To a solution of 490-1 (100 mg, 0.29 mmol) and nitromethane (1.5 mL) in DMSO (2 mL) was added K₂CO₃ (48 mg, 0.34 mmol) at 0° C. The mixture was stirred at r.t. overnight. The mixture was filtered. To the filtrate was added water (5 mL) and the mixture was extracted with EA (10 mL*2). The combined organic layers were washed with brine (10 mL), dried over Na₂SO₄, filtered and concentrated. The residue was purified by FCC (PE/EA=1/1) to give 490-2 (80 mg, 68%) as an off-white solid. MS (ESI) m/z 405 [M+H]⁺.

Step 2 was performed according to the procedure outlined for preparation of 208-3.

### Step 3. 3-((4-chloro-1-methyl-1H-pyrazol-5-yl)methyl)-2-((6-thioxo-5-oxa-7-azaspiro[3.4]octan-2-yl)methyl)isoindolin-1-one (490)

A mixture of 490-3 (20 mg, 0.05 mmol) and di(1H-imidazol-1-yl)methanethione (14 mg, 0.08 mmol) in ACN (2 mL) was stirred at 70° C. for 2 h. The mixture was diluted with water (5 mL) and extracted with EA (10 mL*2). The combined organic layers were washed with brine (10 mL), dried over Na₂SO₄, filtered and concentrated. The residue was purified by prep-HPLC to give 490 (4 mg, 15%) as a white solid. MS (ESI) m/z 417 [M+H]⁺. ¹H NMR (400 MHz, CD₃OD) δ 7.78-7.76 (m, 1H), 7.52-7.49 (m, 3H), 6.77-6.75 (m, 1H), 5.00 (dd, J=8.8, 5.6 Hz, 1H), 4.16 (dd, J=14.4, 6.8 Hz, 1H), 3.78 (s, 2H), 3.62 (dd, J=14.8, 5.7 Hz, 1H), 3.58 (s, 3H), 3.43 (dd, J=14.8, 6.4 Hz, 1H), 2.88 (dd, J=14.8, 8.8 Hz, 1H), 2.56-2.36 (m, 5H).

### 6-methyl-5-((3-oxo-2-((6-oxo-5-oxa-7-azaspiro[3.4]octan-2-yl)methyl)isoindolin-1-yl)methyl)pyrimidine-4-carbonitrile (491)

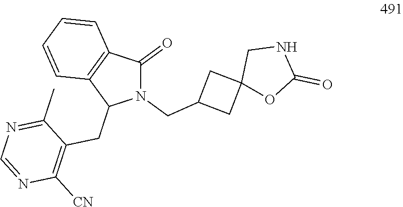 [SMILES: Cc1ncnc(C#N)c1CC1c2ccccc2C(=O)N1CC1CC2(CNC(=O)O2)C1]

The title compound 491 was prepared according to the procedure described for compound 270 as a white solid (20 mg, 25%). MS (ESI) m/z 404 [M+H]⁺. ¹H NMR (400 MHz, DMSO-d₆) δ 9.20 (s, 1H), 7.73 (d, J=6.8 Hz, 1H), 7.51-7.45 (m, 3H), 6.52 (d, J=6.8 Hz, 1H), 4.98-4.97 (m, 1H), 4.05-4.02 (m, 1H), 3.90-3.87 (m, 1H), 3.51 (s, 2H), 3.49-3.39 (m, 1H), 2.73-2.67 (m, 1H), 2.57 (s, 3H), 2.31-2.12 (m, 5H).

The intermediate 491-4 was prepared as follows:

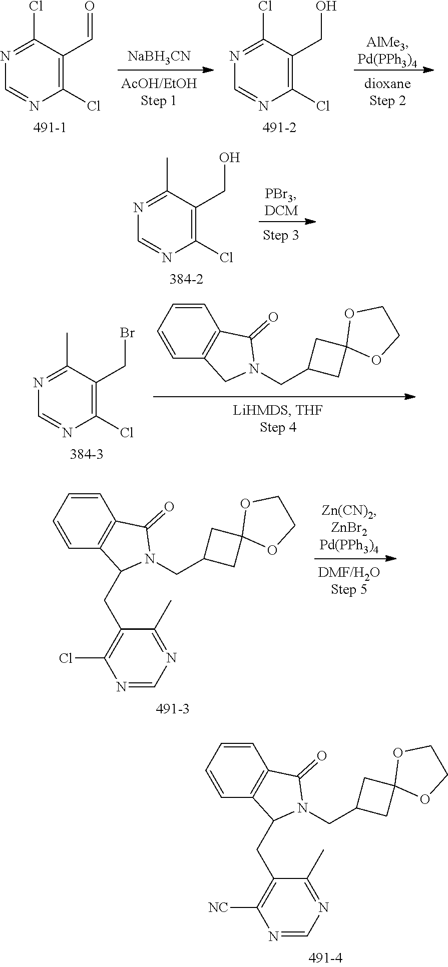

### Step 1. (4,6-dichloropyrimidin-5-yl)methanol (491-2)

To a solution of 4,6-dichloropyrimidine-5-carbaldehyde (8.0 g, 45.2 mmol) in EtOH (80 mL) were added AcOH (8 mL) and NaBH₃CN (4.2 g, 67.8 mmol) at 0° C. The mixture was stirred at r.t. overnight. To the mixture was added water (50 mL) and extracted with EA (100 mL*3), washed with brine (50 mL), dried over anhydrous Na₂SO₄, filtered and concentrated. The residue was purified by FCC (PE/EA=3/1) to give 491-2 (6.6 g, 82%) as a white solid. MS (ESI) m/z 179 [M+H]⁺.

### Step 2. (4-chloro-6-methylpyrimidin-5-yl)methanol (384-2)

A mixture of 491-2 (6.6 g, 36.9 mmol), AlMe₃ (20.3 mL, 40.6 mmol, 2M in hexane) and Pd(PPh₃)₄(4.2 g, 3.7 mmol) in dioxane (100 mL) was stirred at 80° C. overnight. The mixture was diluted with water (50 mL) and extracted with EA (100 mL*3). The combined organic layers were washed with brine (100 mL), dried over anhydrous Na₂SO₄, filtered and concentrated. The residue was purified by column chromatography on silica gel (PE/EA=2/1) to give 384-2 (985 mg, 17%) as a yellow solid. MS (ESI) m/z 159 [M+H]⁺.

Step 3 was performed according to the procedure outlined for preparation of 263-3.

Step 4 was performed according to the procedure outlined for preparation of Int 12-2.

### Step 5. 5-((2-((5,8-dioxaspiro[3.4]octan-2-yl)methyl)-3-oxoisoindolin-1-yl)methyl)-6-methylpyrimidine-4-carbonitrile (491-4)

A mixture of 491-3 (634 mg, 1.59 mmol), Zn(CN)₂ (372 mg, 3.18 mmol), ZnBr₂ (23 mg, 0.11 mmol) and Pd(PPh₃)₄(92 mg, 0.08 mmol) in DMF/H₂O (15 mL/2 drop) was stirred at 110° C. overnight. The mixture was diluted with water (50 mL), extracted with EA (30 mL*3). The combined organic layers were washed with water (50 mL*2) and brine (50 mL), dried over anhydrous Na₂SO₄, filtered and concentrated. The residue was purified by column chromatography on silica gel (PE/EA=1/1) to give 491-4 (600 mg, 97%) as a yellow solid. MS (ESI) m/z 391 [M+H]⁺.

### 5-methyl-4-((3-oxo-2-((6-oxo-5-oxa-7-azaspiro[3.4]octan-2-yl)methyl)isoindolin-1-yl)methyl)nicotinonitrile (492)

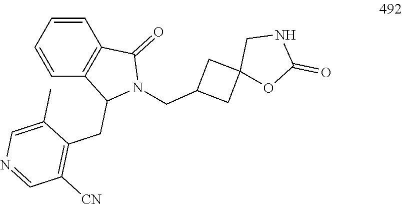 [SMILES: Cc1cncc(C#N)c1CC1c2ccccc2C(=O)N1CC1CC2(CNC(=O)O2)C1]

The title compound 492 was prepared according to the procedure described for compound 270 as a white solid (127 mg, 42%). MS (ESI) m/z 403 [M+H]⁺. ¹H NMR (400 MHz, DMSO-d₆) δ 8.88 (s, 1H), 8.74 (s, 1H), 7.73 (d, J=7.2 Hz, 1H), 7.55-7.37 (m, 2H), 6.44 (d, J=8.0 Hz, 1H), 4.95 (dd, J=10.0, 6.0 Hz, 1H), 4.01 (dd, J=14.4, 6.4 Hz, 1H), 3.81 (dd, J=13.2, 5.6 Hz, 1H), 3.51 (s, 2H), 3.38 (dd, J=14.1, 5.5 Hz, 1H), 2.71 (dd, J=13.3, 9.9 Hz, 1H), 2.43-2.34 (m, 1H), 2.33-2.19 (m, 3H), 2.25 (s, 3H), 2.15-2.03 (m, 1H).

The intermediate 492-2 was prepared as follows:

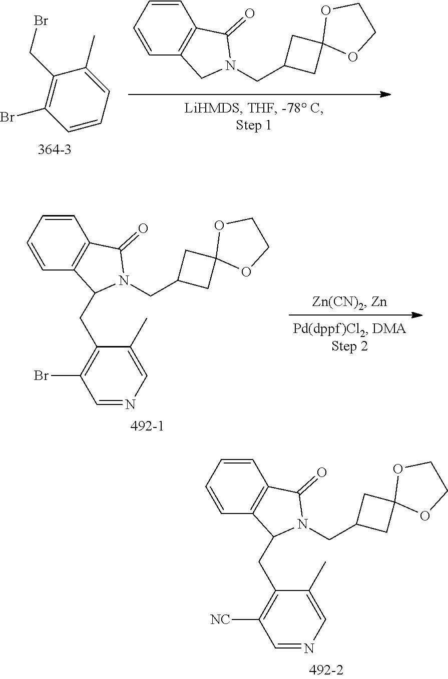

Step 1 was performed according to the procedure outlined for preparation of Int 12-2.

Step 2 was performed according to the procedure outlined for preparation of 284-2.

### 2-((1-((1,3-dimethyl-6-oxo-1,6-dihydropyridin-2-yl)methyl)-3-oxoisoindolin-2-yl)methyl)-5-oxa-7-azaspiro[3.4]octan-6-one (493)

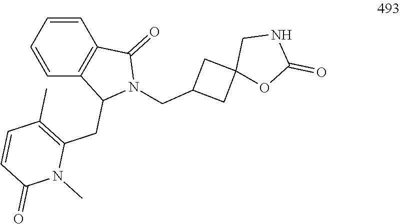 [SMILES: Cc1ccc(=O)n(C)c1CC1c2ccccc2C(=O)N1CC1CC2(CNC(=O)O2)C1]

The title compound 493 was prepared according to the procedure described for compound 451 as a white solid (40 mg, 13%). MS (ESI) m/z 408 [M+H]⁺. ¹H NMR (400 MHz, CDCl₃) δ 7.87 (d, J=7.5 Hz, 1H), 7.49 (t, J=7.4 Hz, 1H), 7.41 (td, J=7.6, 1.3 Hz, 1H), 7.21 (d, J=9.3 Hz, 1H), 6.67 (d, J=7.6 Hz, 1H), 6.61 (d, J=9.3 Hz, 1H), 5.45 (s, 1H), 4.75 (dd, J=9.5, 6.6 Hz, 1H), 4.18 (dd, J=15.0, 3.4 Hz, 1H), 3.62 (s, 2H), 3.51 (dd, J=14.4, 6.5 Hz, 1H), 3.38 (s, 3H), 3.23 (dd, J=15.2, 4.3 Hz, 1H), 2.72 (dd, J=14.4, 9.7 Hz, 1H), 2.51-2.26 (m, 5H), 1.91 (s, 3H).

The intermediate 493-4 was prepared as follows:

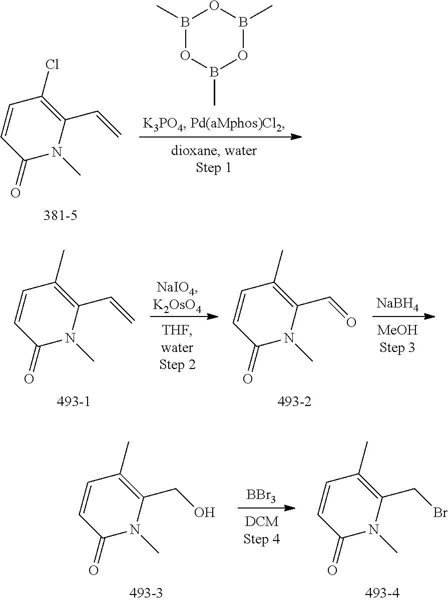

Step 1 was performed according to the procedure outlined for preparation of 348-3.

Step 2 was performed according to the procedure outlined for preparation of 381-6.

Step 3 was performed according to the procedure outlined for preparation of 164-2.

Step 4 was performed according to the procedure outlined for preparation of 263-3.

### 2-((1-((1-methyl-6-oxo-1,6-dihydropyridin-2-yl)methyl)-3-oxoisoindolin-2-yl)methyl)-5-oxa-7-azaspiro[3.4]octan-6-one (494)

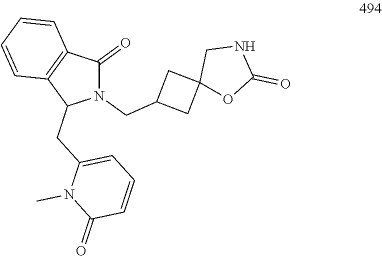 [SMILES: Cn1c(CC2c3ccccc3C(=O)N2CC2CC3(CNC(=O)O3)C2)cccc1=O]

The title compound 494 was prepared according to the procedure described for compound 493 as a white solid (65 mg, 22%). MS (ESI) m/z 394 [M+H]⁺. ¹H NMR (400 MHz, CDCl₃) δ 7.86 (d, J=7.4 Hz, 1H), 7.54-7.40 (m, 2H), 7.30 (dd, J=9.1, 6.9 Hz, 1H), 6.87 (d, J=7.4 Hz, 1H), 6.60 (d, J=9.1 Hz, 1H), 6.06 (d, J=6.0 Hz, 1H), 5.93 (s, 1H), 4.75 (dd, J=9.2, 5.5 Hz, 1H), 4.16 (dd, J=14.0, 4.1 Hz, 1H), 3.62 (s, 2H), 3.49 (dd, J=14.7, 5.5 Hz, 1H), 3.44 (s, 3H), 3.31-3.24 (m, 1H), 2.68 (dd, J=14.7, 9.2 Hz, 1H), 2.47-2.32 (m, 5H).

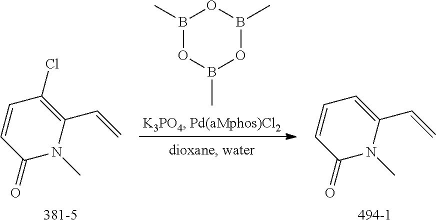

The intermediate 494-1 was a byproduct in the preparation of 493-1.

### 4-chloro-1-methyl-5-((3-oxo-2-((6-oxo-5-oxa-7-azaspiro[3.4]octan-2-yl)methyl)isoindolin-1-yl)methyl)-1H-pyrazole-3-carbonitrile (495)

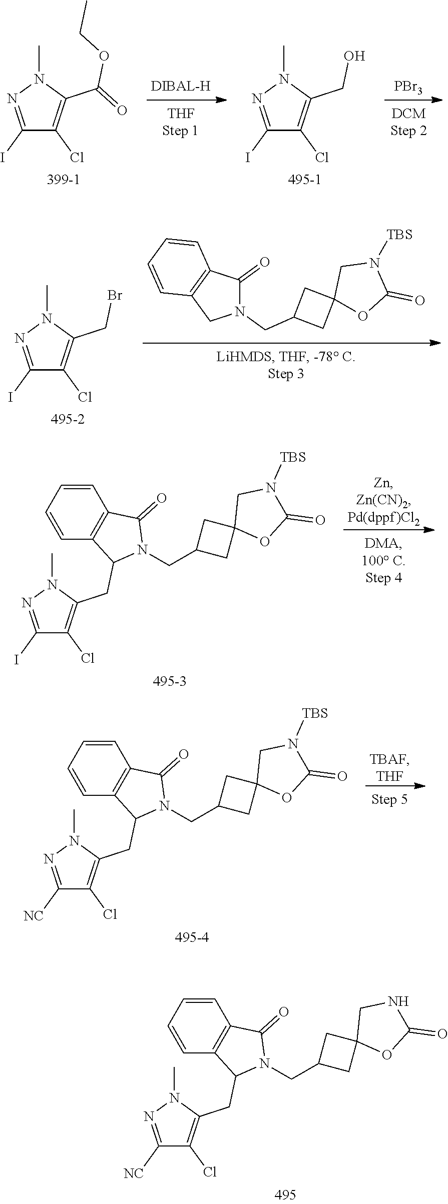

### Step 1. (4-chloro-3-iodo-1-methyl-1H-pyrazol-5-yl)methanol (495-1)

To a solution of 399-1 (3 g, 9.55 mmol) in THF (10 mL) was added DIBAL-H (30 mL, 30 mmol) at 0° C. The resulting mixture was stirred for 1 h at 0° C. The reaction was quenched with H₂O (1.2 mL), 15% NaOH (1.2 mL), and H₂O (3 mL), and added MgSO₄ (10 g), and after filtration, the filtrate was concentrated to give 495-1 (1.8 g, 69%) as a colorless oil. MS (ESI) m/z 273 [M+H]⁺.

Step 2 was performed according to the procedure outlined for preparation of 263-3.

Step 3 was performed according to the procedure outlined for preparation of Int 12-2.

Step 4 was performed according to the procedure outlined for preparation of 284-2.

Step 5 was performed according to the procedure outlined for step 3 for preparation of 445 to afford 495 as a white solid (20 mg, 21%). MS (ESI) m/z 426 [M+H]⁺. ¹H NMR (400 MHz, CDCl₃) δ 7.88 (d, J=7.4 Hz, 1H), 7.58-7.42 (m, 2H), 6.49 (d, J=7.5 Hz, 1H), 5.16 (s, 1H), 4.82 (dd, J=9.9, 5.2 Hz, 1H), 4.25 (dd, J=14.3, 5.2 Hz, 1H), 3.63 (s, 2H), 3.58 (dd, J=14.6, 5.2 Hz, 1H), 3.51 (s, 3H), 3.26 (dd, J=14.4, 6.4 Hz, 1H), 2.61 (dd, J=14.5, 9.9 Hz, 1H), 2.53-2.35 (m, 5H).

### 2-((1-((4-chloro-1-methyl-1H-pyrazol-5-yl)methyl)-4,6-difluoro-3-oxoisoindolin-2-yl)methyl)-5-oxa-7-azaspiro[3.4]octan-6-one (496)

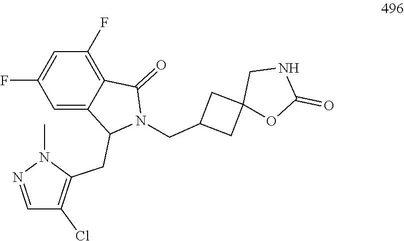 [SMILES: Cn1ncc(Cl)c1CC1c2cc(F)cc(F)c2C(=O)N1CC1CC2(CNC(=O)O2)C1]

The title compound 496 was prepared according to the procedure described for compound 307 as a white solid (34 mg, 27.2%). MS (ESI) m/z 437 [M+H]⁺. ¹H NMR (400 MHz, DMSO-d₆) δ 7.55 (s, 1H), 7.49 (s, 1H), 7.36 (td, J=9.8, 2.0 Hz, 1H), 6.58 (dd, J=8.2, 2.1 Hz, 11H), 4.92 (dd, J=7.8, 5.9 Hz, 1H), 3.93 (dd, J=14.2, 6.0 Hz, 1H), 3.71 (s, 3H), 3.52 (dd, J=15.2, 6.0 Hz, 1H), 3.49 (s, 2H), 3.25 (dd, J=14.2, 6.2 Hz, 1H), 3.02 (dd, J=14.9, 7.9 Hz, 1H), 2.39-2.23 (m, 2H), 2.23-2.11 (m, 2H), 2.11-2.02 (m, 11H).

### 2-((3-((4-chloro-1-methyl-1H-pyrazol-5-yl)methyl)-5-(3-(dimethylamino)azetidin-1-yl)-1-oxoisoindolin-2-yl)methyl)-5-oxa-7-azaspiro[3.4]octan-6-one (497)

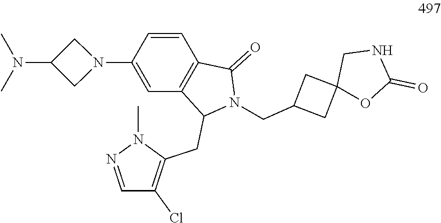 [SMILES: CN(C)C1CN(c2ccc3c(c2)C(Cc2c(Cl)cnn2C)N(CC2CC4(CNC(=O)O4)C2)C3=O)C1]

The title compound 497 was prepared according to the procedure described for compound 463 as a white solid (84 mg, 72.0%, last 2 steps). MS (ESI) m/z 499 [M+H]⁺. ¹H NMR (400 MHz, CDCl₃) δ 7.64 (d, J=8.3 Hz, 1H), 7.50 (s, 1H), 6.42 (dd, J=8.3, 2.0 Hz, 1H), 5.46 (s, 1H), 5.35 (d, J=2.0 Hz, 1H), 4.70 (dd, J=9.8, 5.6 Hz, 1H), 4.15 (dd, J=14.4, 5.4 Hz, 1H), 3.95-3.89 (m, 2H), 3.74-3.70 (m, 2H), 3.67-3.64 (m, 1H), 3.60 (s, 2H), 3.48 (s, 3H), 3.44 (dd, J=14.4, 5.8 Hz, 1H), 3.40-3.31 (m, 1H), 3.10 (dd, J=14.4, 6.9 Hz, 1H), 2.55 (dd, J=14.3, 9.8 Hz, 1H), 2.49-2.32 (m, 4H), 2.29 (s, 6H).

### 2-((3-((4-chloro-1-methyl-1H-pyrazol-5-yl)methyl)-5-(dimethylamino)-1-oxoisoindolin-2-yl)methyl)-5-oxa-7-azaspiro[3.4]octan-6-one (498)

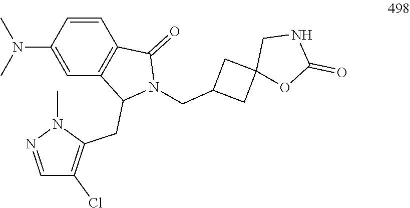 [SMILES: CN(C)c1ccc2c(c1)C(Cc1c(Cl)cnn1C)N(CC1CC3(CNC(=O)O3)C1)C2=O]

The title compound 498 was prepared according to the procedure described for compound 463 as a white solid (69 mg, 56.0%, last 2 steps). MS (ESI) m/z 444 [M+H]⁺. ¹H NMR (400 MHz, CDCl₃) δ 7.65 (d, J=8.6 Hz, 1H), 7.50 (s, 1H), 6.72 (dd, J=8.6, 2.3 Hz, 1H), 5.66 (d, J=2.3 Hz, 1H), 5.56 (s, 1H), 4.71 (dd, J=9.8, 5.6 Hz, 1H), 4.15 (dd, J=14.3, 5.3 Hz, 1H), 3.61 (s, 2H), 3.54-3.41 (m, 4H), 3.12 (dd, J=14.3, 6.8 Hz, 1H), 2.90 (s, 6H), 2.56 (dd, J=14.3, 9.8 Hz, 1H), 2.48-2.26 (m, 5H).

### (2s,4s)-2-((1-((3,4-dichloro-1-methyl-1H-pyrazol-5-yl)methyl)-3-oxoisoindolin-2-yl)methyl)-5-oxa-7-azaspiro[3.4]octan-6-one (499) and (2r,4r)-2-((1-((3,4-dichloro-1-methyl-1H-pyrazol-5-yl)methyl)-3-oxoisoindolin-2-yl)methyl)-5-oxa-7-azaspiro[3.4]octan-6-one (500)

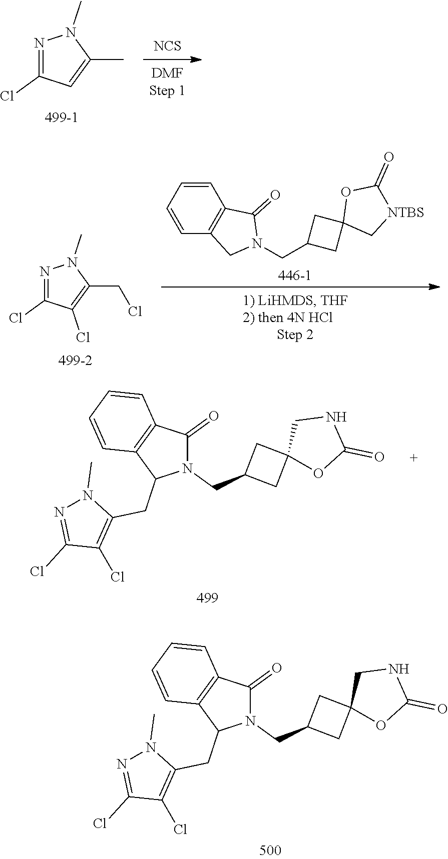

### Step 1. 3,4-dichloro-5-(chloromethyl)-1-methyl-pyrazole (499-2)

A mixture of 3-chloro-1,5-dimethyl-pyrazole (150 mg, 1.15 mmol) and NCS (337 mg, 2.53 mmol) in DMF (4 mL) was stirred at 80° C. for 3 h. The mixture was quenched with water (10 mL) and then extracted with EtOAc (10 mL*3). The organics were combined and dried (Na₂SO₄) before concentrated to dryness. The residue was purified with flash (PE/EtOAc=10/1) to give 499-2 (160 mg, 69.8%) as a white solid. MS (ESI) m/z 199 [M+H]⁺.

### Step 2. (2s,4s)-2-((1-((3,4-dichloro-1-methyl-1H-pyrazol-5-yl)methyl)-3-oxoisoindolin-2-yl)methyl)-5-oxa-7-azaspiro[3.4]octan-6-one (499) and (2r,4r)-2-((1-((3,4-dichloro-1-methyl-1H-pyrazol-5-yl)methyl)-3-oxoisoindolin-2-yl)methyl)-5-oxa-7-azaspiro[3.4]octan-6-one (500)

To a solution of 446-1 (50 mg, 0.13 mmol) in THF (1 ml) was added LiHMDS (0.16 mmol) at −78° C. under N₂. The mixture was stirred for 30 min. Then to the mixture was added 499-2 (28 mg, 0.14 mmol) in THF (0.5 mL). This suspension was allowed to stir for an additional 1 h. Then the mixture was warmed up to r.t. and added 4N HCl (1.5 mL), and then stirred overnight. The mixture was quenched with sat. NaHCO₃ (5 mL) and then extracted with EtOAc. The organic layer was separated, dried over anhydrous Na₂SO₄ and concentrated under vacuum. After separation by pre-HPLC, two isomers were obtained: compound 499 and compound 500. Compound 499 was a white solid (15 mg, 21.1%). MS (ESI) m/z 435 [M+H]⁺. ¹H NMR (400 MHz, CDCl₃) δ 7.87 (dd, J=7.3, 1.5 Hz, 1H), 7.54-7.41 (m, 2H), 6.60 (d, J=7.3 Hz, 1H), 4.98 (s, 1H), 4.82 (dd, J=10.0, 5.3 Hz, 1H), 4.23 (dd, J=14.4, 5.4 Hz, 1H), 3.62 (s, 2H), 3.50 (dd, J=14.5, 5.3 Hz, 11H), 3.41 (s, 3H), 3.24 (dd, J=14.4, 6.5 Hz, 11H), 2.55 (dd, J=14.4, 10.0 Hz, 1H), 2.50-2.30 (m, 5H). Compound 500 was a white solid (1 mg, 1.4%). MS (ESI) m/z 435 [M+H]⁺. ¹H NMR (400 MHz, CDCl₃) δ 7.87 (dd, J=7.0, 1.7 Hz, 1H), 7.53-7.45 (m, 2H), 6.65 (d, J=7.3 Hz, 1H), 4.89-4.77 (m, 2H), 4.13 (dd, J=14.6, 9.6 Hz, 1H), 3.77-3.60 (m, 2H), 3.47 (m, 4H), 3.29 (dd, J=14.4, 5.5 Hz, 1H), 2.82-2.75 (m, 2H), 2.64-2.50 (m, 2H), 2.28-1.96 (m, 2H).

### 3-((4-chloro-1-methyl-1H-pyrazol-5-yl)methyl)-2-(3-(1-methyl-1H-pyrazol-4-yl)prop-2-yn-1-yl)isoindolin-1-one (501)

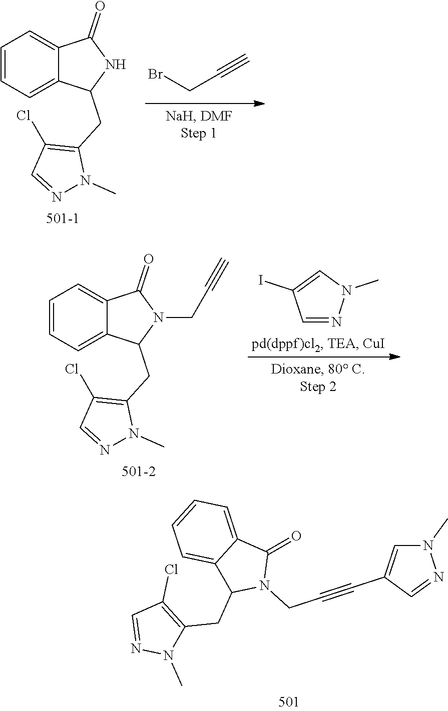

The intermediate 501-1 was prepared according to the procedure outlined for Int 12.

Step 1 was performed according to the procedure outlined for preparation of 222-2.

### Step 2. 3-((4-chloro-1-methyl-1H-pyrazol-5-yl)methyl)-2-(3-(1-methyl-1H-pyrazol-4-yl)prop-2-yn-1-yl)isoindolin-1-one (501)

To a solution of 501-2 (50 mg, 0.2 mmol) and 4-iodo-1-methyl-1H-pyrazole (82 mg, 0.4 mmol) in dried dioxane (5 mL) were added Pd(dppf)Cl₂ (81 mg, 0.1 mmol), TEA (48 mg, 0.4 mmol) and CuI (48 mg, 0.4 mmol). The mixture was stirred at 80° C. for 3h. To the mixture was added water (5 mL) and the mixture was extracted with ethyl acetate (2*10 mL). The combined organic layer was dried over anhydrous Na₂SO₄ and concentrated. The residue was purified by Prep-HPLC to give 501 (3 mg, 7%) as a white solid. MS (ESI) m/z 380 [M+H]⁺. ¹H NMR (400 MHz, CDCl₃) δ 7.91-7.84 (m, 1H), 7.59-7.39 (m, 5H), 6.70 (d, J=7.0 Hz, 1H), 5.15-5.11 (m, 1H), 5.14 (d, J=17.9 Hz, 1H), 4.18 (d, J=18.0 Hz, 1H), 3.88 (s, 3H), 3.60 (s, 3H), 3.60 (dd, J=14.4, 6.0 Hz, 1H), 2.76 (dd, J=14.4, 9.4 Hz, 1H).

### 2-benzyl-3-((4-chloro-1-methyl-1H-pyrazol-5-yl)methyl)isoindolin-1-one (502)

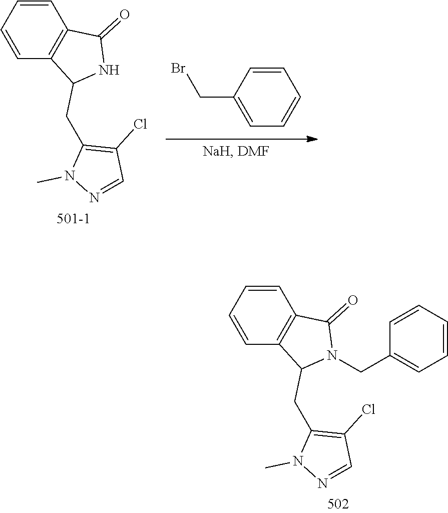 [SMILES: Cn1ncc(Cl)c1CC1NC(=O)c2ccccc21 ; BrCc1ccccc1 ; Cn1ncc(Cl)c1CC1c2ccccc2C(=O)N1Cc1ccccc1]

The title compound 502 was prepared according to the procedure described for Int 5 as a white solid (2 mg, 1.5%). MS (ESI) m/z 352 [M+H]⁺. ¹H NMR (400 MHz, CDCl₃) δ 7.92 (d, J=7.5 Hz, 1H), 7.51-7.40 (m, 3H), 7.36-7.24 (m, 5H), 6.61 (d, J=7.5 Hz, 1H), 5.43 (d, J=15.1 Hz, 1H), 4.65 (dd, J=9.3, 5.9 Hz, 1H), 4.21 (d, J=15.1 Hz, 1H), 3.42 (s, 3H), 3.39 (dd, J=14.4, 6.0 Hz, 1H), 2.66 (dd, J=14.4, 9.3 Hz, 1H).

### 3-((4-chloro-1-methyl-1H-pyrazol-5-yl)methyl)-2-(4-methylbenzyl)isoindolin-1-one (503)

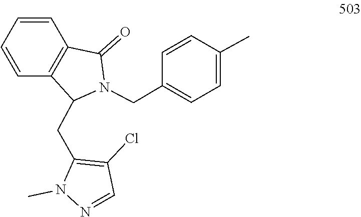 [SMILES: Cc1ccc(CN2C(=O)c3ccccc3C2Cc2c(Cl)cnn2C)cc1]

The title compound 503 was prepared according to the procedure described for compound 502 as a white solid (2 mg, 2%). MS (ESI) m/z 366 [M+H]⁺. ¹H NMR (400 MHz, CDCl₃) δ 7.91 (d, J=7.5 Hz, 1H), 7.51-7.44 (m, 2H), 6.41 (td, J=7.5, 1.3 Hz, 1H), 7.18-7.10 (m, 4H), 6.59 (d, J=7.5 Hz, 1H), 5.38 (d, J=15.0 Hz, 1H), 4.64 (dd, J=9.3, 6.0 Hz, 1H), 4.17 (d, J=15.1 Hz, 1H), 3.43 (s, 3H), 3.40 (d, J=14.2, 6.0 Hz, 1H), 2.65 (dd, J=14.5, 9.4 Hz, 1H), 2.32 (s, 3H).

### (E)-3-((4-chloro-1-methyl-1H-pyrazol-5-yl)methyl)-2-(2-fluoro-3-(3-methyl-1H-pyrazol-4-yl)allyl)isoindolin-1-one (504)

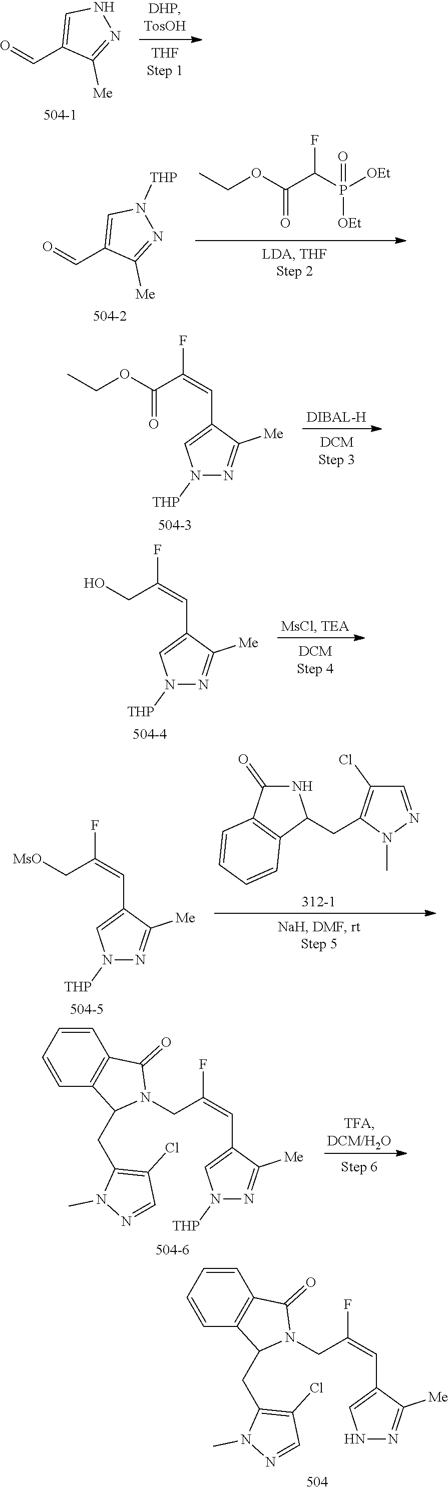

### Step 1. 3-methyl-1-(tetrahydro-2H-pyran-2-yl)-1H-pyrazole-4-carbaldehyde (504-2)

To a solution of 3-methyl-1H-pyrazole-4-carbaldehyde (1.0 g, 9.1 mmol) in THF (30 mL) were added DHP (1.5 g, 18.2 mmol) and TsOH (170 mg, 0.9 mmol) at r.t. The mixture was stirred at r.t. for 16 h. The mixture was concentrated and purified by FCC (PE/EA=10/1) to give 504-2 (1.2 g, 77%) as a yellow oil. MS (ESI) m/z 195 [M+H]⁺.

### Step 2 ethyl (E)-2-fluoro-3-(3-methyl-1-(tetrahydro-2H-pyran-2-yl)-1H-pyrazol-4-yl)acrylate (504-3)

To a solution of ethyl 2-(diethoxyphosphoryl)-2-fluoroacetate (823 mg, 3.4 mmol) in THF (30 mL) was added LDA (1.9 mL, 3.7 mmol, 2M in THF) at −25° C. and stirred for 1 h. To the mixture was added 504-2 (600 mg, 3.1 mmol) at −78° C. The mixture was stirred at r.t. for 16 h. The mixture was poured into ice-water (30 mL), and extracted with EA (30 mL*2). The combined organic layers were washed with brine (30 mL), dried over Na₂SO₄, filtered and concentrated. The residue was purified by FCC (PE/EA=5/1) to give 504-3 (400 mg, 46%) as a yellow solid. MS (ESI) m/z 283 [M+H]⁺.

### Step 3. (E)-2-fluoro-3-(3-methyl-1-(tetrahydro-2H-pyran-2-yl)-1H-pyrazol-4-yl)prop-2-en-1-ol (504-4)

To a solution of 504-3 (800 mg, 2.8 mmol) in DCM (30 mL) was added DIBAL-H (6.2 mL, 6.2 mmol, 1M in hexane) at 0° C. The mixture was stirred at r.t. for 16 h. The mixture was quenched with aq. NH₄Cl (30 mL) in an ice-bath, and extracted with DCM (30 mL*2). The combined organic layers were washed with brine (30 mL), dried over Na₂SO₄, filtered and concentrated. The residue was purified by FCC (PE/EA=5/1 to 3/1) to give 504-4 (450 mg, 67%) as a yellow oil. MS (ESI) m/z 241 [M+H]⁺.

### Step 4. (E)-2-fluoro-3-(3-methyl-1-(tetrahydro-2H-pyran-2-yl)-1H-pyrazol-4-yl)allyl methanesulfonate (504-5)

To a solution of 504-4 (270 mg, 1.1 mmol) in DCM (10 mL) were added TEA (0.5 mL, 3.3 mmol) and MsCl (270 mg, 1.1 mmol) in an ice-bath. The reaction mixture was stirred at r.t. for 3 h. The mixture was poured into water (10 mL) and extracted with DCM (20 mL*2). The combined organic layers were washed with brine (20 mL), dried over Na₂SO₄, filtered and concentrated to give 504-5 (350 mg, crude) as a yellow oil. MS (ESI) m/z 319 [M+H]⁺.

Step 5 was performed according to the procedure outlined for preparation of Int 5.

### Step 6. (E)-3-((4-chloro-1-methyl-1H-pyrazol-5-yl)methyl)-2-(2-fluoro-3-(3-methyl-1H-pyrazol-4-yl)allyl)isoindolin-1-one (504)

A mixture of 504-6 (60 mg, 0.12 mmol) in TFA/DCM/H₂O (2 mL/5 mL/1 mL) was stirred at r.t. for 3 h. The mixture was concentrated, added ice-water (3 mL), adjusted to pH at 8 with aq. NaHCO₃ and extracted with DCM (20 mL*2). The combined organic layers were washed with brine (20 mL), dried over Na₂SO₄, filtered and concentrated. The residue was purified by FCC (DCM/MeOH=15/1) to afford 504 (22 mg, 45%) as a white solid. MS (ESI) m/z 400 [M+H]⁺. ¹H NMR (400 MHz, DMSO-d₆) δ 12.64 (d, J=25.2 Hz, 1H), 7.74-7.50 (m, 5H), 6.96 (d, J=6.8 Hz, 1H), 5.85 (d, J=15.6 Hz, 0.5H), 5.74 (d, J=15.6 Hz, 0.5H), 4.97-4.88 (m, 2H), 4.06-3.95 (m, 1H), 3.69 (s, 3H), 3.55 (dd, J=14.8, 6.0 Hz, 1H), 3.03 (dd, J=15.2, 8.4 Hz, 1H), 2.25 (s, 1.8H), 2.19 (s, 1.2H).

### 3-((3-bromopyridin-2-yl)methyl)-2-((5-hydroxypyrimidin-2-yl)methyl)isoindolin-1-one (505)

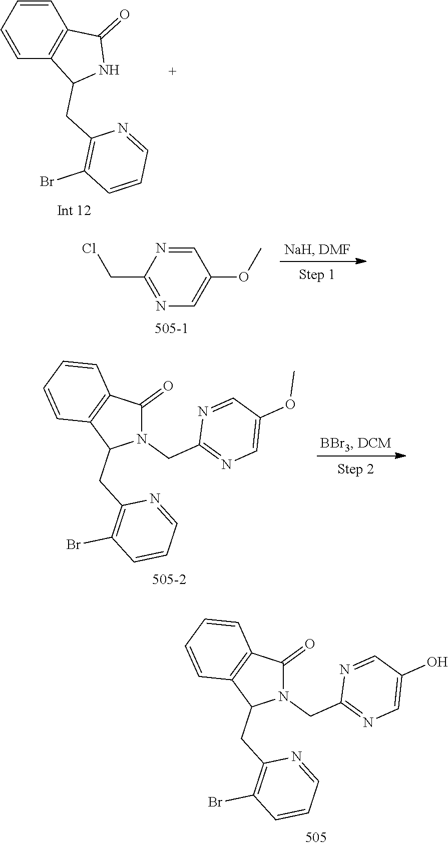

Step 1 was performed according to the procedure outlined for preparation of Int 5.

Step 2 was performed according to the procedure outlined for step 2 for preparation of compound 14 to afford 505 as a white solid (11 mg, 22.6%). MS (ESI) m/z 411 [M+H]⁺. ¹H NMR (400 MHz, DMSO-d₆) δ 8.47 (dd, J=8.4, 1.6 Hz, 1H), 8.18 (s, 2H), 8.02 (dd, J=8.4, 1.6 Hz, 1H), 7.72 (dd, J=6.4, 1.6 Hz, 1H), 7.53-7.50 (m, 2H), 7.24 (dd, J=8.0, 4.4 Hz, 1H), 7.13 (d, J=6.0 Hz, 1H), 5.42 (t, J=6.4 Hz, 11H), 5.10 (d, J=16.8 Hz, 11H), 4.48 (d, J=16.8 Hz, 11H), 3.56 (dd, J=15.2, 6.4 Hz, 11H), 3.27 (dd, J=15.2, 6.4 Hz, 1H).

### 2-((1-((4-chloro-1-methyl-1H-pyrazol-5-yl)methyl)-4-fluoro-6-methyl-3-oxoisoindolin-2-yl)methyl)-5-oxa-7-azaspiro[3.4]octan-6-one (506)

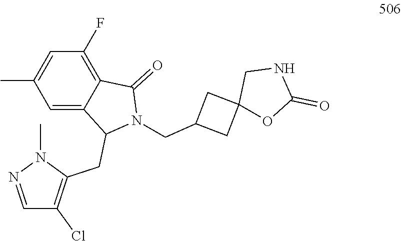 [SMILES: Cc1cc(F)c2c(c1)C(Cc1c(Cl)cnn1C)N(CC1CC3(CNC(=O)O3)C1)C2=O]

The title compound 506 was prepared according to the procedure described for compound 307 as a white solid (18 mg, 26%). MS (ESI) m/z 433 [M+H]⁺. ¹H NMR (400 MHz, DMSO-d₆) δ 7.55 (s, 1H), 7.49 (s, 1H), 7.10 (d, J=10.6 Hz, 1H), 6.47 (s, 1H), 4.84 (dd, J=8.0, 5.7 Hz, 1H), 3.93 (dd, J=14.2, 6.0 Hz, 1H), 3.65 (s, 3H), 3.50 (dd, J=14.9, 5.8 Hz, 1H), 3.49 (s, 2H), 3.25 (dd, J=14.2, 6.1 Hz, 1H), 2.94 (dd, J=14.8, 8.1 Hz, 1H), 2.39-2.33 (m, 1H), 2.30 (s, 3H), 2.29-2.12 (m, 3H), 2.10-2.02 (m, 1H).

The intermediate 506-3 was prepared as follows:

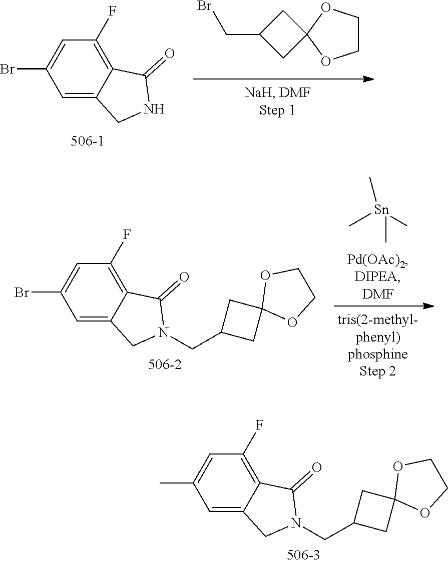

Step 1 was performed according to the procedure outlined for preparation of Int 5.

### Step 2. 2-((5,8-dioxaspiro[3.4]octan-2-yl)methyl)-7-fluoro-5-methylisoindolin-1-one (506-3)

To a solution of 506-2 (200 mg, 0.56 mmol) in DMF (5 mL) were added Pd(OAc)₂ (10 mg, 0.06 mmol), tris(2-methylphenyl)phosphine (37 mg, 0.12 mmol), DIPEA (363 mg, 2.81 mmol) and tetramethylstannane (110 mg, 0.62 mmol). The reaction mixture was stirred at 100′˜C for 6 h. The reaction mixture was diluted with EA and washed with water. The organic layer was separated, dried over anhydrous Na₂SO₄ and concentrated under vacuum. The residue was purified by TLC to afford 506-3 (90 mg, 55.2%) as a yellow oil. MS (ESI) m/z 292 [M+H]⁺.

### 2-((1-((3-(azetidin-1-ylmethyl)-4-chloro-1-methyl-1H-pyrazol-5-yl)methyl)-3-oxoisoindolin-2-yl)methyl)-5-oxa-7-azaspiro[3.4]octan-6-one (508)

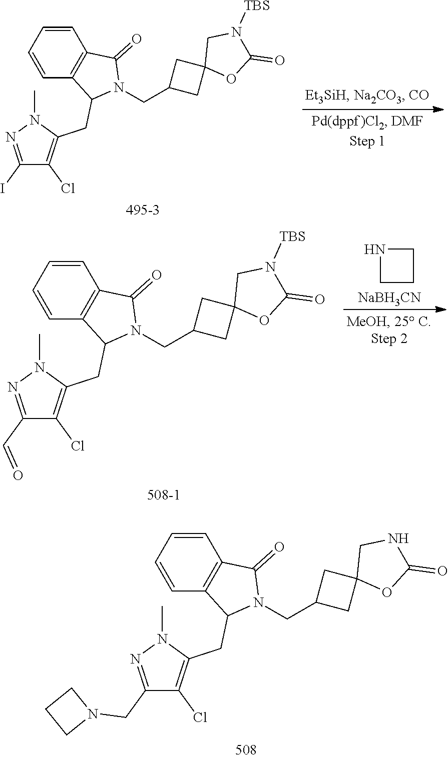

Step 1 was performed according to the procedure outlined for preparation of 273-1.

### Step 2. 2-((1-((3-(azetidin-1-ylmethyl)-4-chloro-1-methyl-1H-pyrazol-5-yl)methyl)-3-oxoisoindolin-2-yl)methyl)-5-oxa-7-azaspiro[3.4]octan-6-one (508)

To a solution of 508-1 (150 mg, 0.27 mmol) and azetidine (57 mg, 1 mmol) in MeOH (5 mL) was added NaBH₃CN (31 mg, 0.5 mmol), The resulting mixture was stirred for 12 h at 25° C. The reaction mixture was concentrated under reduced pressure. The residue was purified by reverse phase flash chromatography eluting with ACN/H₂O to afford 508 (8 mg, 6%) as a white solid. MS (ESI) m/z 470 [M+H]⁺. ¹H NMR (400 MHz, CDCl₃) δ 7.85 (d, J=7.1 Hz, 1H), 7.52-7.40 (m, 2H), 6.60 (d, J=7.3 Hz, 1H), 5.24 (s, 1H), 4.84 (dd, J=9.4, 5.5 Hz, 1H), 4.23 (dd, J=14.9, 4.8 Hz, 1H), 3.95 (s, 2H), 3.83 (t, J=7.7 Hz, 4H), 3.62 (s, 4H), 3.46 (s, 3H), 3.48 (dd, J=13.8, 5.0 Hz, 1H), 3.20 (dd, J=14.7, 5.5 Hz, 1H), 4.84 (dd, J=14.4, 9.5 Hz, 1H), 2.50-2.25 (m, 7H).

### 2-((1-((4-chloro-1-methyl-3-yl)-1H-pyrazol-5-yl)methyl)-3-oxoisoindolin-2-yl)methyl)-5-oxa-7-azaspiro[3.4]octan-6-one (509)

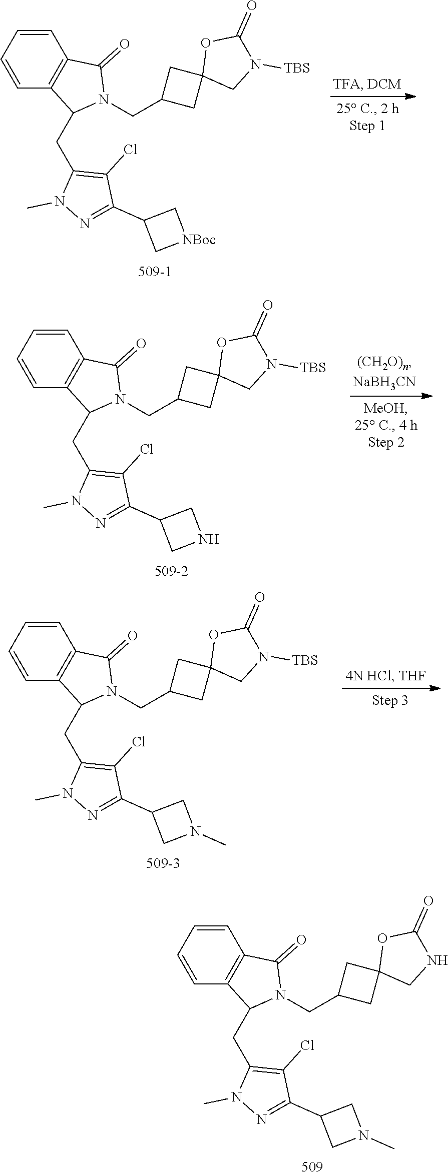

The intermediate 509-1 was prepared according to the procedure described for 451-1 using 438-4 as a starting material.

Step 1 was performed according to the procedure outlined for preparation of Int 12.

Step 2 was performed according to the procedure outlined for step 2 for the preparation of 508.

### Step 3. 2-((1-((4-chloro-1-methyl-3-(1-methylazetidin-3-yl)-1H-pyrazol-5-yl) methyl)-3-oxoisoindolin-2-yl)methyl)-5-oxa-7-azaspiro[3.4]octan-6-one (509)

To a stirred solution of 509-3 (50 mg, 0.08 mmol) in THF (1 mL) was added 4N HCl (1 mL) at r.t. The resulting mixture was stirred for 4 h at 25° C. Then the mixture was quenched with NaHCO₃, diluted with water (10 mL), and extracted with EtOAc (3*10 mL). The combined organic layers were washed with brine (10 mL), dried over anhydrous Na₂SO₄, and after filtration, the filtrate was concentrated under reduced pressure. The residue was purified by reverse phase flash chromatography eluting with ACN/H₂O to afford 509 (8 mg, 21%) as a white solid. MS (ESI) m/z 470 [M+H]⁺. ¹H NMR (400 MHz, CDCl₃) δ 7.86 (d, J=7.2 Hz, 1H), 7.58-7.38 (m, 2H), 6.60 (d, J=7.2 Hz, 1H), 5.33 (s, 1H), 4.81 (dd, J=9.1, 5.7 Hz, 1H), 4.39 (t, J=8.8 Hz, 2H), 4.22 (dd, J=14.3, 3.9 Hz, 1H), 4.11 (p, J=8.2 Hz, 1H), 3.90 (t, J=8.5 Hz, 1H), 3.81 (t, J=8.4 Hz, 1H), 3.61 (s, 2H), 3.46 (s, 3H), 3.42 (dd, J=14.5, 5.5 Hz, 1H), 3.17 (dd, J=13.9, 4.7 Hz, 1H), 2.76 (s, 3H), 2.61 (dd, J=14.5, 9.3 Hz, 1H), 2.47-2.30 (m, 5H).

### 2-((6-chloro-1-((4-chloro-1-methyl-1H-pyrazol-5-yl)methyl)-4-fluoro-3-oxoisoindolin-2-yl)methyl)-5-oxa-7-azaspiro[3.4]octan-6-one (510)

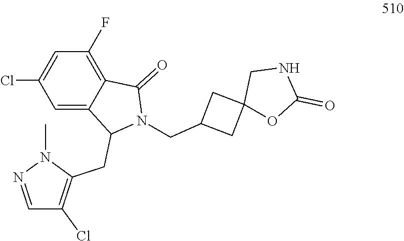 [SMILES: Cn1ncc(Cl)c1CC1c2cc(Cl)cc(F)c2C(=O)N1CC1CC2(CNC(=O)O2)C1]

The title compound 510 was prepared according to the procedure described for compound 307 as a white solid (9 mg, 12.4%). MS (ESI) m/z 453 [M+H]⁺. ¹H NMR (400 MHz, DMSO-d₆) δ 7.56 (s, 1H), 7.53 (dd, J=9.3, 1.6 Hz, 1H), 7.50 (s, 1H), 6.80 (d, J=1.5 Hz, 1H), 4.92 (t, J=6.8 Hz, 1H), 3.93 (dd, J=14.2, 6.1 Hz, 1H), 3.71 (s, 3H), 3.53 (dd, J=15.0, 5.8 Hz, 1H), 3.49 (s, 2H), 3.28 (dd, J=14.1, 6.2 Hz, 1H), 3.02 (dd, J=14.9, 7.9 Hz, 1H), 2.39-2.32 (m, 1H), 2.31-2.23 (m, 1H), 2.22-2.12 (m, 2H), 2.11-2.02 (m, 1H).

The intermediate 510-3 was prepared as follows:

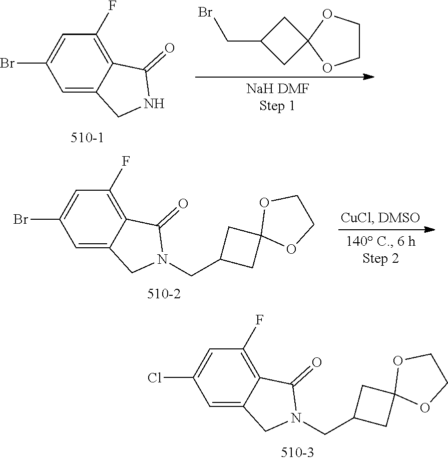

Step 1 was performed according to the procedure outlined for preparation of Int 5.

Step 2 was performed according to the procedure outlined for preparation of 474.

### 2-((3-((4-chloro-1-methyl-1H-pyrazol-5-yl)methyl)-4-fluoro-1-oxoisoindolin-2-yl)methyl)-5-oxa-7-azaspiro[3.4]octan-6-one (511)

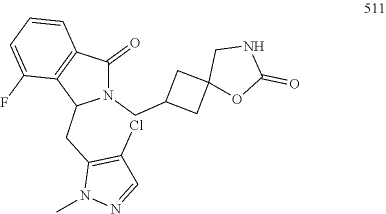 [SMILES: Cn1ncc(Cl)c1CC1c2c(F)cccc2C(=O)N1CC1CC2(CNC(=O)O2)C1]

The title compound 511 was prepared according to the procedure described for compound 451 as a white solid (85 mg, 60%). MS (ESI) m/z 419 [M+H]⁺.H NMR (400 MHz, CDCl₃) δ 7.67 (d, J=7.5 Hz, 1H), 7.54-7.47 (m, 1H), 7.45 (s, 1H), 7.26-7.21 (m, 1H), 5.17 (s, 1H), 4.99 (dd, J=7.3, 5.5 Hz, 1H), 4.16 (dd, J=14.3, 6.0 Hz, 1H), 3.68 (s, 3H), 3.57 (s, 2H), 3.28 (dd, J=15.1, 5.6 Hz, 1H), 3.11 (dd, J=15.1, 7.3 Hz, 1H), 2.86 (dd, J=14.3, 6.9 Hz, 1H), 2.39-2.11 (m, 5H).

511-1 was prepared according to the procedure described for preparation of 446-1.

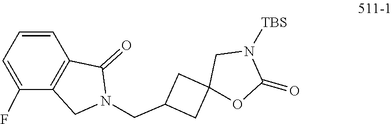

### 2-((5-((2-oxa-6-azaspiro[3.3]heptan-6-yl)methyl)-3-((4-chloro-1-methyl-1H-pyrazol-5-yl)methyl)-1-oxoisoindolin-2-yl)methyl)-5-oxa-7-azaspiro[3.4]octan-6-one (512)

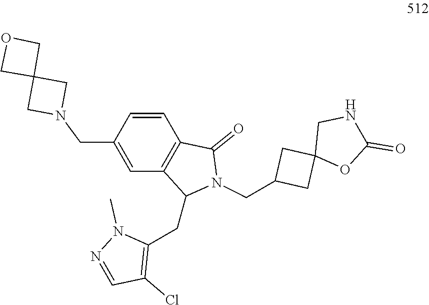 [SMILES: Cn1ncc(Cl)c1CC1c2cc(CN3CC4(COC4)C3)ccc2C(=O)N1CC1CC2(CNC(=O)O2)C1]

The title compound 512 was prepared according to the procedure described for compound 461 as a white solid (10 mg, 11%, last 2 steps). MS (ESI) m/z 512 [M+H]⁺. ¹H NMR (400 MHz, CDCl₃) δ 7.80 (d, J=7.8 Hz, 1H), 7.52 (s, 1H), 7.40 (d, J=7.7 Hz, 1H), 6.54 (s, 1H), 4.96 (s, 1H), 4.82 (dd, J=9.1, 6.0 Hz, 1H), 4.77-4.73 (m, 4H), 4.20 (dd, J=14.4, 5.3 Hz, 1H), 3.61 (s, 2H), 3.60-3.51 (m, 2H), 3.49 (s, 3H), 3.45-3.40 (m, 4H), 3.13 (dd, J=14.4, 6.6 Hz, 1H), 2.66 (dd, J=14.5, 9.1 Hz, 1H), 2.50-2.17 (m, 6H).

The intermediate 512-2 was prepared as follows:

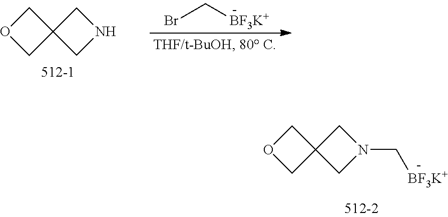

To a solution of 2-oxa-6-azaspiro[3.3]heptane (500 mg, 5.04 mmol) in t-BuOH (5 mL) and THF (20 mL) was added potassium (bromomethyl)trifluoroborate (2.02 g, 10.09 mmol). The reaction mixture was stirred for 2 h at 80° C. and was filtered. The filtrate was concentrated under vacuum. The residue was used for the next step without further purification as a yellow solid (510 mg).

### 3-((3-bromopyridin-2-yl)methyl)-2-((6-methoxypyridin-3-yl)methyl)isoindolin-1-one (513)

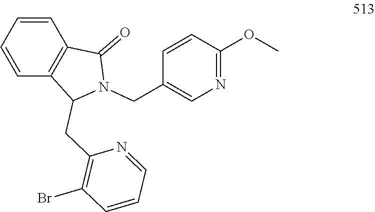 [SMILES: COc1ccc(CN2C(=O)c3ccccc3C2Cc2ncccc2Br)cn1]

The title compound 513 was prepared according to the procedure described for Int 5 (78 mg, 18.4%) as a yellow solid. MS (ESI) m/z 424 [M+H]⁺. ¹H NMR (400 MHz, DMSO-d₆) δ 8.56 (dd, J=4.6, 1.5 Hz, 1H), 8.05 (dd, J=8.1, 1.5 Hz, 1H), 7.89 (d, J=2.4 Hz, 1H), 7.76-7.69 (m, 1H), 7.55-7.45 (m, 3H), 7.30 (dd, J=8.1, 4.6 Hz, 1H), 7.08-7.01 (m, 1H), 6.74 (d, J=8.5 Hz, 1H), 5.15 (t, J=6.5 Hz, 1H), 4.96 (d, J=15.4 Hz, 1H), 4.39 (d, J=15.4 Hz, 1H), 3.81 (s, 3H), 3.59 (dd, J=15.1, 6.0 Hz, 1H), 3.22 (dd, J=15.1, 7.1 Hz, 1H).

### tert-butyl 3-(3-((4-chloro-1-methyl-1H-pyrazol-5-yl)methyl)-1-oxo-2-((6-oxo-5-oxa-7-azaspiro[3.4]octan-2-yl)methyl)isoindolin-5-yl)azetidine-1-carboxylate (514)

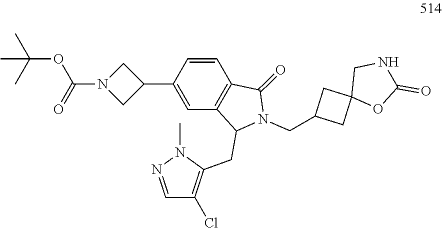 [SMILES: Cn1ncc(Cl)c1CC1c2cc(C3CN(C(=O)OC(C)(C)C)C3)ccc2C(=O)N1CC1CC2(CNC(=O)O2)C1]

The title compound 514 was prepared according to the procedure described for compound 473 as a white solid (30 mg, 55.0%, last 2 steps). MS (ESI) m/z 556 [M+H]⁺. ¹H NMR (400 MHz, CDCl₃) δ 7.83 (d, J=7.8 Hz, 1H), 7.51 (s, 1H), 7.48-7.41 (m, 1H), 6.49 (s, 1H), 5.16 (s, 1H), 4.82 (dd, J=9.2, 6.0 Hz, 1H), 4.31 (td, J=8.6, 1.8 Hz, 2H), 4.20 (dd, J=14.4, 4.9 Hz, 1H), 3.86 (td, J=8.2, 5.7 Hz, 2H), 3.73-3.66 (m, 1H), 3.61 (s, 2H), 3.49 (s, 3H), 3.44 (dd, J=14.7, 6.3 Hz, 1H), 3.14 (dd, J=14.4, 6.2 Hz, 1H), 2.67 (dd, J=14.4, 9.2 Hz, 1H), 2.49-2.27 (m, 5H), 1.48 (s, 9H).

### (E)-3-((4-chloro-1-methyl-1H-pyrazol-5-yl)methyl)-2-(2-fluoro-3-(1H-1,2,3-triazol-4-yl)allyl)isoindolin-1-one (515)

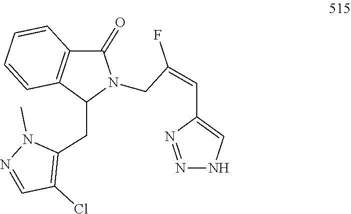 [SMILES: Cn1ncc(Cl)c1CC1c2ccccc2C(=O)N1C/C(F)=C\c1c[nH]nn1]

The title compound 515 was prepared according to the procedure described for compound 314 as a white solid (8 mg, 32.2%). MS (ESI) m/z 387 [M+H]⁺. ¹H NMR (400 MHz, CD₃OD) S 7.90 (s, 1H), 7.81 (dd, J=6.4, 1.6 Hz, 1H), 7.55-7.48 (m, 2H), 7.39 (s, 1H), 6.69 (d, J=6.8 Hz, 1H), 6.49 (d, J=18.8 Hz, 1H), 5.26-5.19 (m, 1H), 5.12-5.00 (m, 1H), 4.97 (dd, J=9.4, 5.5 Hz, 1H), 3.72 (dd, J=14.4, 5.6 Hz, 1H), 3.50 (s, 3H), 2.88 (dd, J=14.4, 9.6 Hz, 1H).

The intermediate 515-4 was prepared as follows:

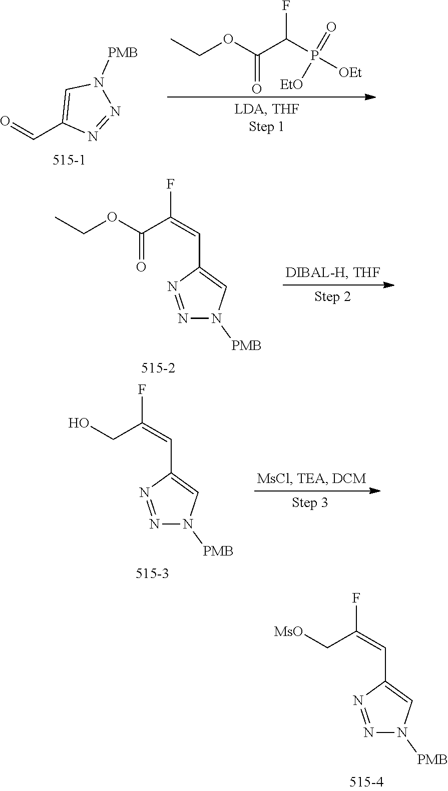

### Step 1. ethyl (E)-2-fluoro-3-(1-(4-methoxybenzyl)-1H-1,2,3-triazol-4-yl)acrylate (515-2)

To a solution of ethyl 2-(diethoxyphosphoryl)-2-fluoroacetate (266 mg, 1.10 mmol) in THF (5 mL) was added LDA (0.6 mL, 1.2 mmol, 2M in THF) at −25′T and stirred for 1 h. The mixture was cooled to −78′C and 1-(4-methoxybenzyl)-1H-1,2,3-triazole-4-carbaldehyde (217 mg, 1.00 mmol) was added. The mixture was stirred at r.t. for 16 h. The mixture was quenched with aq. NH₄Cl (10 mL), and extracted with EA (10 mL*2). The combined organic layers were washed with brine (10 mL), dried over Na₂SO₄, filtered and concentrated. The residue was purified by FCC (PE/EA=5/1) to give 515-2 (190 mg, 69%) as a yellow oil. MS (ESI) m/z 306 [M+H]⁺.

### Step 2. (E)-2-fluoro-3-(1-(4-methoxybenzyl)-1H-1,2,3-triazol-4-yl)prop-2-en-1-ol (515-3)

To a solution of 515-2 (190 mg, 0.62 mmol) in THF (5 mL) was added DIBAL-H (1.4 mL, 1.37 mmol, 1M in hexane) at 0° C. The mixture was stirred at r.t. overnight under N₂. The mixture 20 was quenched with aq. NH₄Cl (10 mL), and extracted with EA (10 mL*3). The combined organic layers were washed with brine (20 mL), dried over Na₂SO₄, filtered and concentrated. The residue was purified by FCC (PE/EA=1/1) to give 515-3 (120 mg, 75%) as a colorless oil. MS (ESI) m/z 264 [M+H]⁺.

### Step 3. (E)-2-fluoro-3-(1-(4-methoxybenzyl)-1H-1,2,3-triazol-4-yl)allylmethanesulfonate (515-4)

To a solution of 515-3 (120 mg, 0.46 mmol) and TEA (137 mg, 1.37 mmol) in DCM (10 mL) was added MsCl (104 mg, 0.91 mmol) at 0′C. The mixture was stirred at r.t. overnight under N₂. The mixture was quenched with aq. NH₄Cl (10 mL), and extracted with DCM (10 mL*3). The combined organic layers were washed with brine (20 mL), dried over Na₂SO₄, filtered and concentrated to give 515-4 (109 mg, 100%) as a yellow solid. MS (ESI) m/z 342 [M+H]⁺.

### (E)-3-((4-chloro-1-methyl-1H-pyrazol-5-yl)methyl)-2-(3-(5-fluoro-1H-1,2,3-triazol-4-yl)allyl)isoindolin-1-one (516)

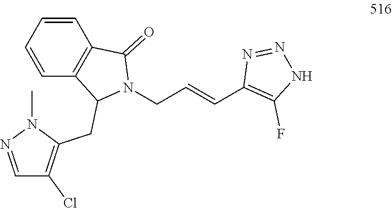 [SMILES: Cn1ncc(Cl)c1CC1c2ccccc2C(=O)N1C/C=C/c1nn[nH]c1F]

The title compound 516 was prepared according to the procedure described for compound 515 as a white solid (9 mg, 39%). MS (EST) m/z 387 [M+H]⁺. ¹H NMR (400 MHz, CD₃OD) 5 7.83-7.80 (m, 1H), 7.55-7.50 (m, 3H), 6.89-6.86 (m, 1H), 6.52 (d, J=16.4 Hz, 1H), 6.38-6.31 (m, 1H), 5.03 (dd, J=8.4, 6.0 Hz, 1H), 4.92-4.86 (m, 1H), 4.05 (dd, J=16.0, 7.6 Hz, 1H), 3.61 (s, 3H), 3.55 (dd, J=14.4, 6.0 Hz, 1H), 2.97 (dd, J=14.8, 8.8 Hz, 1H).

The intermediate 516-7 was prepared as follows:

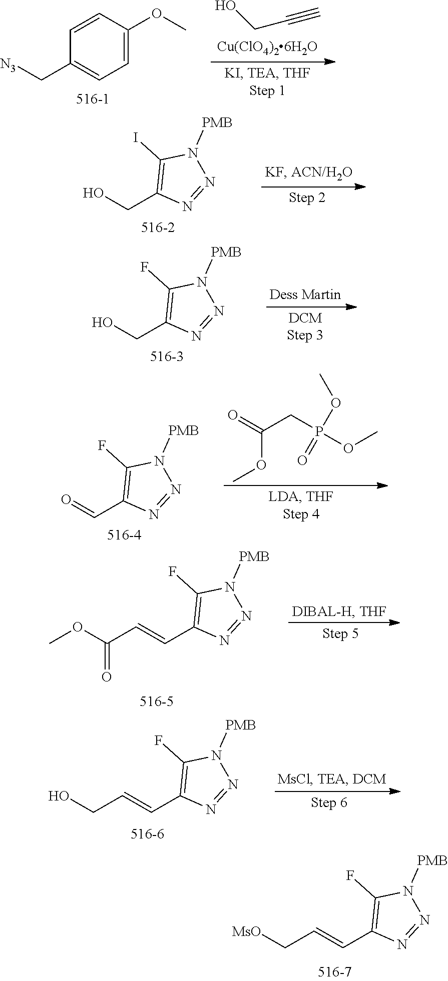

### Step 1. (5-iodo-1-(4-methoxybenzyl)-1H-1,2,3-triazol-4-yl)methanol (516-2)

To a solution of I-(azidomethyl)-4-methoxybenzene (1.00 g, 6.13 mmol), Cu(ClO₄)₂ 6H₂O (4.55 g, 12.26 mmol) and KI (4.07 g, 24.54 mmol) in THF (50 mL) were added TEA (1.24 g, 12.27 mmol) and prop-2-yn-1-ol (343 mg, 6.13 mmol) at r.t. The mixture was stirred at r.t. overnight under N₂. The mixture was poured into water (50 mL), extracted with EA (50 mL*2). The combined organic layers were washed with NH₃H₂O (20 mL) and H₂O (20 mL), dried over Na₂SO₄, filtered and concentrated and purified by FCC (DCM/MeOH=20/1) to give 516-2 (1.7 g, 81%) as a white solid. MS (ESI) m/z 346 [M+H]⁺.

### Step 2. (5-fluoro-1-(4-methoxybenzyl)-1H-1,2,3-triazol-4-yl)methanol (516-3)

A mixture of 516-2 (345 mg, 1.00 mmol) and KF (290 mg, 5.00 mmol) in ACN/H₂O (4 mL/4 mL) was stirred at 160′C for 10 min under microwave. The mixture was poured into H₂O (10 mL), and extracted with EA (15 mL*2). The combined organic layers were dried over Na₂SO₄, filtered and concentrated. The residue was purified by FCC (PE/EA=1/1 to 1/2) to give 516-3 (600 mg, 63%) as a yellow solid. MS (ESI) m/z 238 [M+H]⁺.

Step 3 was performed according to the procedure outlined for preparation of 403-5.

Step 4 was performed according to the procedure outlined for preparation of 515-2.

Step 5 was performed according to the procedure outlined for preparation of 515-3.

Step 6 was performed according to the procedure outlined for preparation of 515-4.

### 3-((4-chloro-1-methyl-1H-pyrazol-5-yl)methyl)-2-(3-fluoro-4-methoxybenzyl)isoindolin-1-one (517)

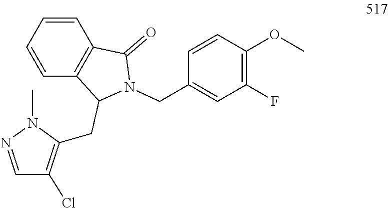 [SMILES: COc1ccc(CN2C(=O)c3ccccc3C2Cc2c(Cl)cnn2C)cc1F]

The title compound 517 was prepared according to the procedure described for compound 502 as a white solid (12 mg, 14%). MS (ESI) m/z 400 [M+H]⁺. ¹H NMR (400 MHz, CDCl₃) δ 7.91 (d, J=7.4 Hz, 1H), 7.53-7.39 (m, 3H), 7.04-6.85 (m, 3H), 6.63 (d, J=7.5 Hz, 1H), 5.31 (d, J=15.1 Hz, 1H), 4.67 (dd, J=9.0, 6.2 Hz, 1H), 4.12 (d, J=15.2 Hz, 1H), 3.87 (s, 3H), 3.47 (s, 3H), 3.37 (dd, J=14.5, 6.1 Hz, 1H), 2.67 (dd, J=14.5, 9.1 Hz, 1H).

### 3-((4-chloro-1-methyl-1H-pyrazol-5-yl)methyl)-2-(4-methoxybenzyl)isoindolin-1-one (518)

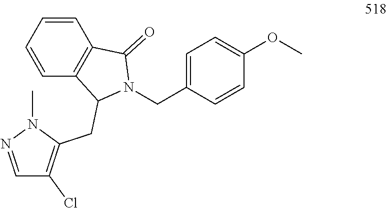 [SMILES: COc1ccc(CN2C(=O)c3ccccc3C2Cc2c(Cl)cnn2C)cc1]

The title compound 518 was prepared according to the procedure described for compound 502 as a white solid (6 mg, 8.9%). MS (ESI) m/z 382 [M+H]⁺. ¹H NMR (400 MHz, CDCl₃) δ 7.91 (d, J=7.5 Hz, 1H), 7.52-7.37 (m, 3H), 7.18 (d, J=8.6 Hz, 2H), 6.86 (d, J=7.4 Hz, 2H), 6.60 (d, J=7.5 Hz, 1H), 5.36 (d, J=15.0 Hz, 1H), 4.64 (dd, J=9.2, 6.0 Hz, 1H), 4.14 (d, J=15.0 Hz, 1H), 3.79 (s, 3H), 3.44 (s, 3H), 3.40 (dd, J=14.5, 6.0 Hz, 1H), 2.65 (dd, J=14.5, 9.3 Hz, 1H).

### (Z)-3-((4-chloro-1-methyl-1H-pyrazol-5-yl)methyl)-2-(3-(3-chloro-1H-pyrazol-4-yl)-2-fluoroallyl)isoindolin-1-one (519)

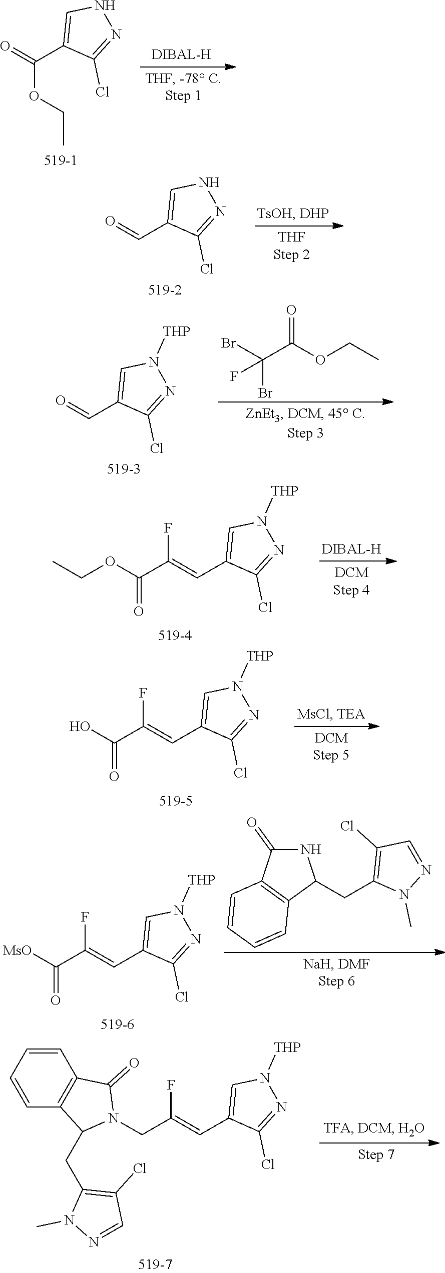

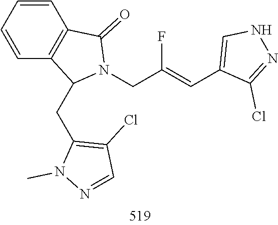 [SMILES: Cn1ncc(Cl)c1CC1c2ccccc2C(=O)N1C/C(F)=C/c1c[nH]nc1Cl]

### Step 1. 3-chloro-1H-pyrazole-4-carbaldehyde (519-2)

To a solution of ethyl 3-chloro-1H-pyrazole-4-carboxylate (2 g, 11.50 mmol) in THF (20 mL) was added DIBAL-H (23.0 mL, 23.0 mmol, 1M in THE) at −78° C. and stirred at −78 TC for 2h. H₂O (1 mL) was added slowly at 0′C, NaOH (1 mL, 15% in H₂O) was added and the resulting mixture was stirred for 3 min. H₂O (2.3 mL) was added and stirred at r.t. for 15 min. Then the mixture was added MgSO₄ (10 g) was added and stirred for 15 min. Filtered by celite and concentrated, the residue was purified by FCC (PE/EA=1/1) to give 519-2 (1.64 g, 99%) as a white solid. MS (ESI) m/z 131 [M+H]⁺.

Step 2 was performed according to the procedure outlined for preparation of 504-2.

### Step 3. ethyl (Z)-3-(3-chloro-1-(tetrahydro-2H-pyran-2-yl)-1H-pyrazol-4-yl)-2-fluoroacrylate (519-4)

To a solution of 3-chloro-1-(tetrahydro-2H-pyran-2-yl)-1H-pyrazole-4-carbaldehyde (700 mg, 3.27 mmol) and ethyl 2,2-dibromo-2-fluoroacetate (1.73 g, 6.54 mmol) in DCM (20 mL) was added ZnEt₂ (13.1 mL, 13.1 mmol, 1 M in THF) dropwise under N₂. The mixture was stirred at 45° C. for 4 h. The mixture was quenched with aq. NH₄Cl (10 mL), and extracted with DCM (10 mL*3). The combined organic layers were washed with brine (20 mL), dried over Na₂SO₄, filtered and concentrated. The residue was purified by FCC (PE/EA=10/1) to give 519-4 (260 mg, 26%) as a yellow solid. MS (ESI) m/z 303 [M+H]⁺.

Step 4 was performed according to the procedure outlined for preparation of 504-4.

Step 5 was performed according to the procedure outlined for preparation of 504-5.

Step 6 was performed according to the procedure outlined for preparation of Int 5.

Step 7 was performed according to the procedure outlined for step 6 for preparation of 504 to afford 519 as a white solid (16 mg, 22%). MS (ESI) m/z 420 [M+H]⁺. ¹H NMR (400 MHz, CD₃OD) b 7.89 (d, J=2.1 Hz, 1H), 7.87-7.78 (m, 1H), 7.56-7.53 (m, 2H), 7.52 (s, 1H), 6.93-6.85 (m, 1H), 5.86 (d, J=38.4 Hz, 1H), 5.08-4.97 (m, 2H), 4.10 (dd, J=26.4, 16.0 Hz, 1H), 3.63 (s, 3H), 3.60 (dd, J=14.7, 6.0 Hz, 1H), 2.99 (dd, J=14.8, 8.6 Hz, 1H).

### N-(2-((1-((4-chloro-1-methyl-1H-pyrazol-5-yl)methyl)-3-oxoisoindolin-2-yl)methyl)-5-oxa-7-azaspiro[3.4]oct-6-en-6-yl)cyanamide (520)

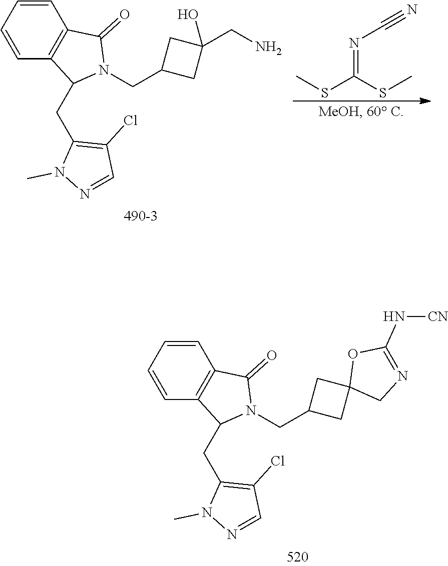

To a solution of 490-3 (100 mg, 0.26 mmol) in MeOH (20 mL) was added dimethyl cyanocarbonimidodithioate (39 mg, 0.26 mmol) at r.t. under N₂. The reaction mixture was stirred at 60 TC for 12 h. The reaction mixture was concentrated and purified by prep-HPLC to give 520 (4 mg, 3.2%) as a white solid. MS (ESI) m/z 447 [M+Na]. ¹H NMR (400 MHz, CD₃OD) δ 7.76-7.78 (m, 1H), 7.49-7.51 (m, 3H), 6.82-6.74 (m, 1H), 4.96-5.00 (m, 1H), 4.10-4.12 (m, 1H), 3.61 (s, 2H), 3.57-3.61 (m, 4H), 3.43-3.34 (m, 3H), 2.86-2.90 (m, 1H), 2.19-2.31 (m, 2H), 1.81-1.89 (m, 1H),

### 2-((3-((4-chloro-1-methyl-1H-pyrazol-5-yl)methyl)-5-fluoro-1-thioxoisoindolin-2-yl)methyl)-5-oxa-7-azaspiro[3.4]octan-6-one (521)

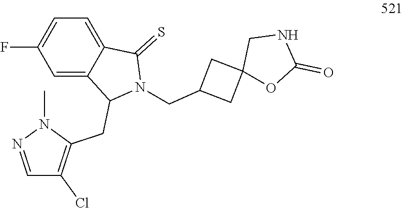 [SMILES: Cn1ncc(Cl)c1CC1c2cc(F)ccc2C(=S)N1CC1CC2(CNC(=O)O2)C1]

The title compound 521 was prepared according to the procedure described for compound 482 as a white solid (4 mg, 6%). MS (ESI) m/z 435 [M+H]⁺. ¹H NMR (400 MHz, CD₃OD) δ 7.96 (dd, J=8.5, 5.1 Hz, 1H), 7.49 (s, 1H), 7.29-7.19 (m, 1H), 6.53 (dd, J=8.4, 2.3 Hz, 1H), 5.23 (dd, J=8.5, 5.8 Hz, 1H), 4.93-4.89 (m, 1H), 3.76-3.67 (m, 2H), 3.65 (s, 3H), 3.64 (s, 2H), 3.01 (dd, J=14.9, 8.6 Hz, 1H), 2.58-2.43 (m, 3H), 2.42-2.37 (m, 2H).

### 2-((1-((4-chloro-1-methyl-3-(morpholinomethyl)-1H-pyrazol-5-yl)methyl)-3-oxoisoindolin-2-yl)methyl)-5-oxa-7-azaspiro[3.4]octan-6-one(522)

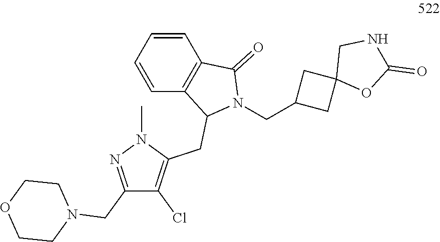 [SMILES: Cn1nc(CN2CCOCC2)c(Cl)c1CC1c2ccccc2C(=O)N1CC1CC2(CNC(=O)O2)C1]

The title compound 522 was prepared according to the procedure described for compound 508 as a white solid (7 mg, 10%). MS (ESI) m/z 500 [M+H]⁺. ¹H NMR (400 MHz, CD₃OD) δ 7.67-7.62 (m, 1H), 7.45-7.36 (m, 2H), 6.91-6.85 (m, 1H), 4.93 (dd, J=7.7, 5.7 Hz, 11H), 4.07 (dd, J=14.3, 5.6 Hz, 1H), 3.73 (s, 2H), 3.66 (t, J=4.8 Hz, 4H), 3.53 (s, 3H), 3.52-3.45 (m, 3H), 3.32 (dd, J=14.5, 5.4 Hz, 1H), 2.92 (dd, J=15.0, 7.8 Hz, 1H), 2.68 (t, J=4.8 Hz, 4H), 2.45-2.35 (m, 1H), 2.30-2.15 (m, 4H).

### 2-((1-((4-chloro-3-(2-(dimethylamino)ethoxy)-1-methyl-1H-pyrazol-5-yl)methyl)-3-oxoisoindolin-2-yl)methyl)-5-oxa-7-azaspiro[3.4]octan-6-one (523)

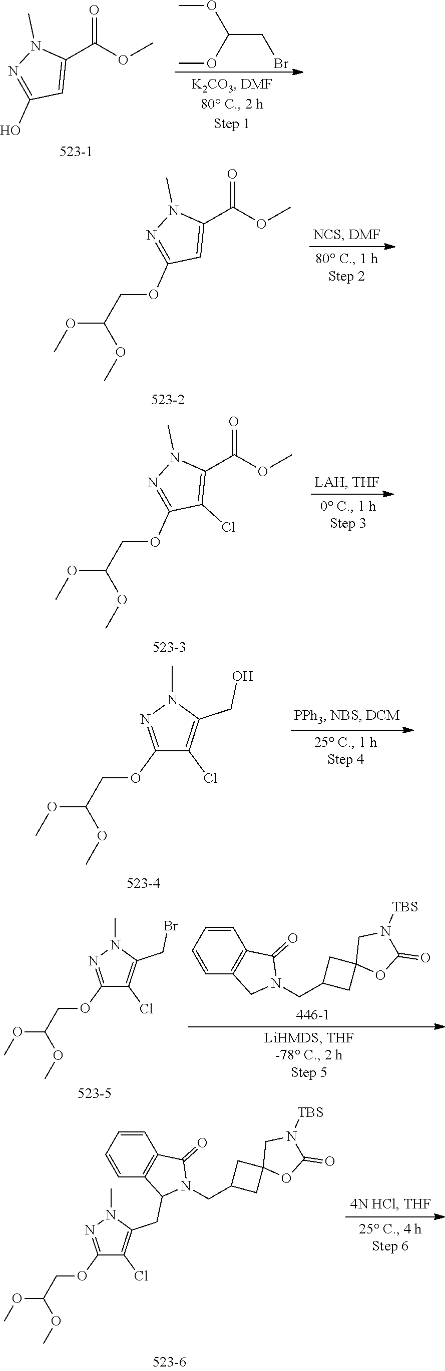

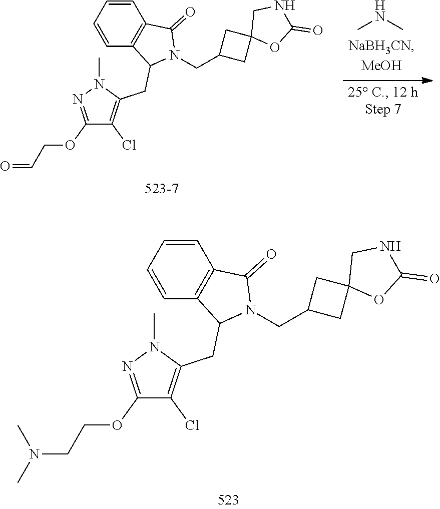

### Step 1. methyl 3-(2,2-dimethoxyethoxy)-1-methyl-1H-pyrazole-5-carboxylate (523-2)

To a solution of methyl 3-hydroxy-1-methyl-IH-pyrazole-5-carboxylate (1 g, 6.4 mmol) and 2-bromo-1,1-dimethoxyethane (1.62 g, 9.6 mmol) in DMF (10 mL) was added K₂CO₃ (1.77 g, 12.82 mmol). The resulting mixture was stirred for 12 h at 80° C. under N₂. The mixture was allowed to cool down to r.t. and diluted with water (20 mL), and extracted with EtOAc (3*20 mL). The combined organic layers were washed with brine (10 mL), dried over anhydrous Na₂SO₄, and after filtration, the filtrate was concentrated under reduced pressure. The residue was purified by silica gel column chromatography, and eluted with (PE/EA=2/1) to afford 523-2 (1.1 g, 70%) as a white solid. MS (ESI) m/z 245 [M+H]⁺.

Step 2 was performed according to the procedure outlined for preparation of 334-2.

Step 3 was performed according to the procedure outlined for preparation of 263-2.

Step 4 was performed according to the procedure outlined for preparation of 333-7.

Step 5 was performed according to the procedure outlined for preparation of Int 12-2.

### Step 6. 2-((4-chloro-1-methyl-5-((3-oxo-2-((6-oxo-5-oxa-7-azaspiro[3.4]octan-2-yl)methyl)isoindolin-1-yl)methyl)-1H-pyrazol-3-yl)oxy)acetaldehyde (523-7)

To a stirred solution of 7-(tert-butyldimethylsilyl)-2-((1-((4-chloro-3-(2,2-dimethoxyethoxy)-1-methyl-1H-pyrazol-5-yl)methyl)-3-oxoisoindolin-2-yl)methyl)-5-oxa-7-azaspiro[3.4]octan-6-one (50 mg, 0.08 mmol) in THE (1 mL) was added 4N HCl (1 mL) at r.t. The resulting mixture was stirred for 4 h at 25° C. Then the mixture was quenched with NaHCO₃, diluted with water (10 mL) and extracted with EtOAc (3*10 mL). The combined organic layers were washed with brine (10 mL), dried over anhydrous Na₂SO₄, and after filtration, the filtrate was concentrated under reduced pressure to afford 523-7 (35 mg, 95%) as a colorless oil. MS (ESI) m/z 459 [M+H]⁺.

### Step 7. 2-((1-((4-chloro-3-(2-(dimethylamino)ethoxy)-1-methyl-1H-pyrazol-5-yl)methyl)-3-oxoisoindolin-2-yl)methyl)-5-oxa-7-azaspiro[3.4]octan-6-one (523)

To a stirred solution of 523-7 (35 mg, 0.07 mmol) in MeOH (5 mL) was added Me₂NH (0.2 mL, 0.4 mmol, 2 mmol/ml in THF). After 1 h, NaBH₃CN (13 mg, 0.2 mmol) was added. The resulting mixture was stirred for 12 h at 25° C. The mixture was extracted with EtOAc (3*10 mL). The combined organic layers were washed with brine (10 mL), dried over anhydrous Na₂SO₄, and after filtration, the filtrate was concentrated under reduced pressure. The residue was purified by reverse phase flash chromatography to afford 523 (2 mg, 6%) as a white solid. MS (ESI) m/z 488 [M+H]⁺. ¹H NMR (400 MHz, CD₃OD) δ 7.69-7.65 (m, 1H), 7.47-7.40 (m, 2H), 6.98-6.92 (m, 1H), 4.89 (dd, J=7.8, 5.9 Hz, 1H), 4.45-4.39 (m, 2H), 4.06 (dd, J=14.2, 5.9 Hz, 1H), 3.53 (s, 2H), 3.40 (s, 3H), 3.38-3.34 (m, 3H), 3.29-3.23 (m, 1H), 2.87 (dd, J=14.9, 7.8 Hz, 1H), 2.79 (s, 6H), 2.43-2.34 (m, 1H), 2.31-2.08 (m, 4H).

### 2-((6-chloro-1-((4-chloro-1-methyl-1H-pyrazol-5-yl)methyl)-3-oxo-1,3-dihydro-2H-pyrrolo[3,4-c]pyridin-2-yl)methyl)-5-oxa-7-azaspiro[3.4]octan-6-one (524)

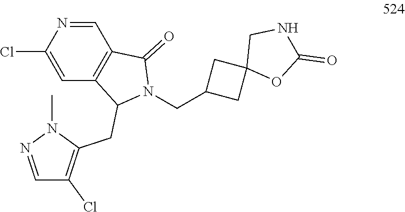 [SMILES: Cn1ncc(Cl)c1CC1c2cc(Cl)ncc2C(=O)N1CC1CC2(CNC(=O)O2)C1]

The title compound 524 was prepared according to the procedure described for compound 451 as a white solid (10 mg, 76%). MS (ESI) m/z 436 [M+H]⁺. ¹H NMR (400 MHz, CDCl₃) δ 8.88 (s, 1H), 7.56 (s, 1H), 6.59 (s, 1H), 4.89 (m, 2H), 4.18 (dd, J=15.2, 5.2 Hz, 1H), 3.61 (s, 5H), 3.49 (dd, J=14.5, 5.7 Hz, 1H), 3.19 (dd, J=14.3, 5.7 Hz, 1H), 2.70 (dd, J=14.6, 9.6 Hz, 1H), 2.48-2.34 (m, 5H).

The intermediate 524-10 was prepared as follows:

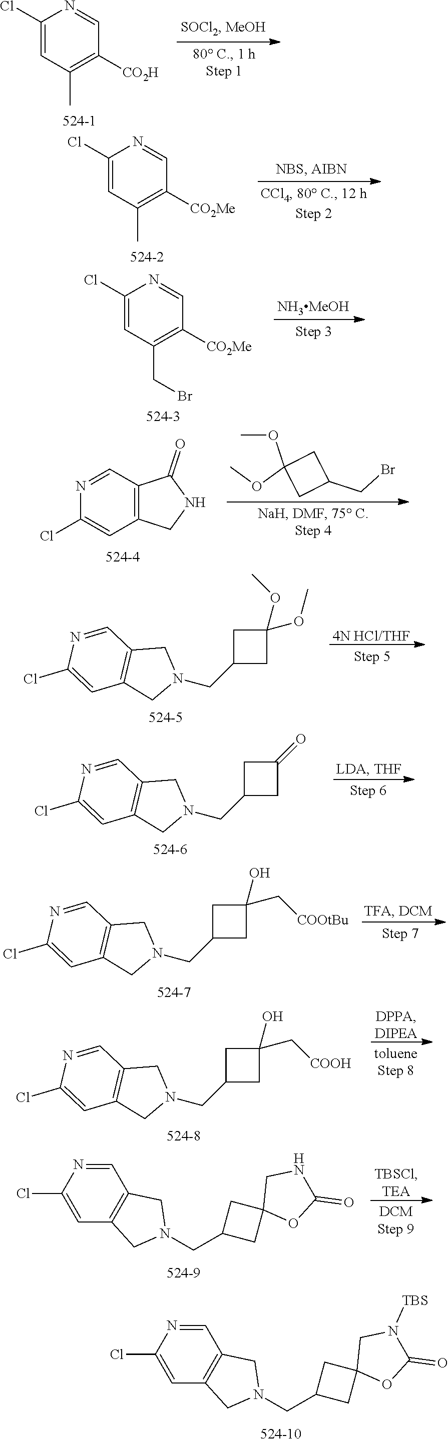

### Step 1: methyl 6-chloro-4-methylnicotinate (524-2)

To a solution of 6-chloro-4-methylnicotinic acid (20 g, 116.9 mmol) in MeOH (50 mL) was added SOCl₂ (50 mL) at 0° C. The resulting mixture was stirred for 1 h at 70° C. The reaction mixture was concentrated under reduced pressure. The residue was purified by silica gel flash column chromatography (PE/EtOAc=10/1) to afford 524-2 (20 g, 92%) as a white solid. MS (ESI) m/z 186 [M+H]⁺.

Step 2 was performed according to the procedure outlined for preparation of 222-1.

### Step 3: 6-chloro-1,2-dihydro-3H-pyrrolo[3,4-c]pyridin-3-one (524-4)

A mixture of methyl 4-(bromomethyl)-6-chloronicotinate (13 g, 49.2 mmol) in NH₃ MeOH (20 mL) was stirred at 60′C for 1 h under sealed tube. To the mixture was added 20 mL of water. The resulting mixture was extracted with EtOAc (20 ml*3). The combined organic phase was successively washed with water (10 mL) and brine (10 mL), dried over anhydrous Na₂SO₄, and after filtration, the filtrate was concentrated under reduced pressure. The residue was purified by silica gel flash column chromatography (EA) to afford 524-4 (6 g, 73%) as a white solid. MS (ESI) m/z 169 [M+H]⁺.

524-10 was prepared according to the procedure described for 446-1 using 524-4 as a starting material.

### (E)-3-((4-chloro-1-methyl-1H-pyrazol-5-yl)methyl)-2-(3-(5-chloro-1H-1,2,3-triazol-4-yl)allyl)isoindolin-1-one (525)

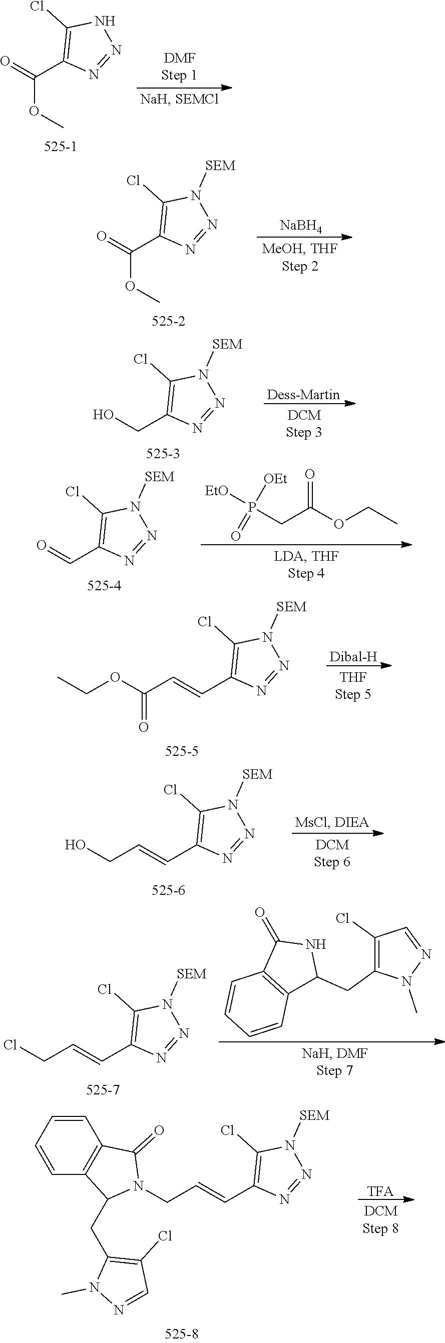

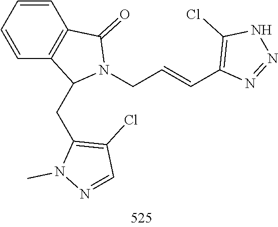 [SMILES: Cn1ncc(Cl)c1CC1c2ccccc2C(=O)N1C/C=C/c1nn[nH]c1Cl]

Step 1 was performed according to the procedure outlined for preparation of Int 4-2.

Step 2 was performed according to the procedure outlined for preparation of 164-2.

Step 3 was performed according to the procedure outlined for preparation of 403-5.

Step 4 was performed according to the procedure outlined for preparation of 515-2.

Step 5 was performed according to the procedure outlined for preparation of 515-3.

Step 6 was performed according to the procedure outlined for preparation of 515-4.

Step 7 was performed according to the procedure outlined for preparation of Int 5.

Step 8 was performed according to the procedure outlined for step 2 for preparation of compound 7 to afford 525 as a white solid (21 mg, 40.6%). MS (ESI) m/z 403 [M+H]⁺. ¹H NMR (400 MHz, DMSO-d₆) δ 7.72-7.70 (m, 1H), 7.54-7.48 (m, 3H), 6.91-6.88 (m, 1H), 6.47 (d, J=16.2 Hz, 1H), 6.25-6.17 (m, 1H), 4.90 (dd, J=8.4, 5.8 Hz, 1H), 4.73-4.68 (m, 1H), 3.92 (dd, J=15.5, 8.0 Hz, 1H), 3.65 (s, 3H), 3.53 (dd, J=1 4.8, 5.9 Hz, 1H), 2.95 (dd, J=14.8, 8.4 Hz, 1H).

### 2-((1-((4-chloro-1,2-dimethyl-1H-imidazol-5-yl)methyl)-3-oxoisoindolin-2-yl)methyl)-5-oxa-7-azaspiro[3.4]octan-6-one (526)

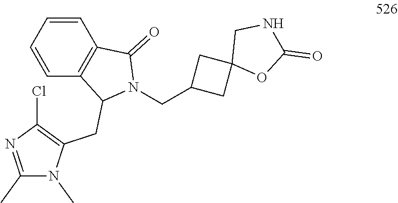 [SMILES: Cc1nc(Cl)c(CC2c3ccccc3C(=O)N2CC2CC3(CNC(=O)O3)C2)n1C]

The title compound 526 was prepared according to the procedure described for compound 451 as a white solid (10 mg, 27%). MS (ESI) m/z 415 [M+H]⁺. ¹H NMR (400 MHz, CD₃OD) δ 7.81-7.70 (m, 1H), 7.57-7.43 (m, 2H), 6.96-6.86 (m, 1H), 4.90 (dd, J=8.7, 5.8 Hz, 1H), 4.13 (dd, J=14.1, 5.4 Hz, 1H), 3.63 (s, 2H), 3.53-3.39 (m, 2H), 3.32 (s, 3H), 2.76 (dd, J=15.2, 8.8 Hz, 1H), 2.48 (m, 1H), 2.40-2.24 (m, 7H).

The intermediate 526-5 was prepared as follows:

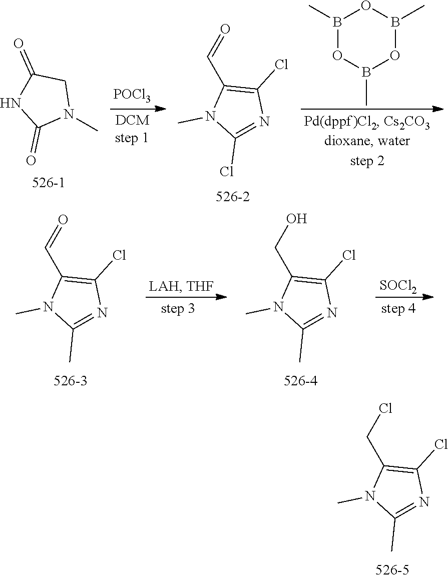

### Step 1: 2,4-dichloro-1-methyl-1H-imidazole-5-carbaldehyde (526-2)

POCl₃ (23 g, 0.15 mol) was added to a dispersion of 1-methylimidazolidine-2,4-dione (3.42 g, 0.03 mol) in 4.38 g of DMF while stirring and cooling to 0-5° C. The reaction mixture was stirred at 90° C. for 8 h. The excess POCl₃ was removed under vacuum. The residue was diluted with 50 ml of water. The mixture was neutralized with crystalline NaHCO₃ to pH=8. The precipitate was filtered off, and the filtrate was extracted with ethyl acetate (3*50 ml). The combined organic layers were washed with water (10 mL) and brine (10 mL), dried over Na₂SO₄, filtered, and concentrated in vacuo. The residue was purified by column chromatography on silica gel (eluting with PE/EA=5/1 to 1/1) to give 526-2 (1.7 g, 22%) as a white solid. MS (ESI) m/z 179 [M+H]f.

Step 2 was performed according to the procedure outlined for preparation of 348-3.

Step 3 was performed according to the procedure outlined for preparation of 263-2.

### Step 4: 4-chloro-5-(chloromethyl)-1,2-dimethyl-1H-imidazole (526-5)

The mixture of (4-chloro-1,2-dimethyl-1H-imidazol-5-yl)methanol (200 mg, 1.2 mmol) in SOCl₂ (3 mL) was stirred at 0° C. for 2 h. The solvent was removed to obtained 526-5 (200 mg, 94%) as a solid. MS (ESI) m/z 179 [M+H]⁺.

### 2-((1-((4-chloro-1,3-dimethyl-1H-pyrazol-5-yl)methyl)-3-oxoisoindolin-2-yl)methyl)-5-oxa-7-azaspiro[3.4]octan-6-one (527)

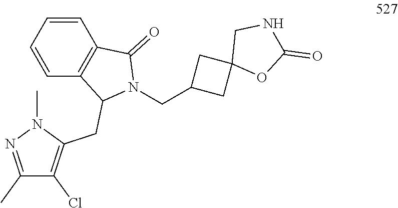 [SMILES: Cc1nn(C)c(CC2c3ccccc3C(=O)N2CC2CC3(CNC(=O)O3)C2)c1Cl]

The title compound 527 was prepared according to the procedure described for compound 451 as a white solid (22 mg, 31%). MS (ESD) m/z 415 [M+H]⁺. ¹H NMR (400 MHz, CDCl₃) δ 7.86 (d, J=7.3 Hz, 1H), 7.52-7.39 (m, 2H), 6.57 (d, J=7.3 Hz, 1H), 5.07 (s, 1H), 4.83 (dd, J=9.9, 5.4 Hz, 1H), 4.26-4.18 (m, 1H), 3.62 (s, 2H), 4.46 (dd, J=14.3, 5.4 Hz, 1H), 3.40 (s, 3H), 3.22 (dd, J=14.6, 5.8 Hz, 1H), 2.54 (dd, J=14.3, 9.9 Hz, 1H), 2.47-2.35 (m, 5H), 2.30 (s, 3H).

### 3-((4-chloro-1-methyl-1H-pyrazol-5-yl)methyl)-2-(4-(difluoromethoxy)benzyl)isoindolin-1-one (528)

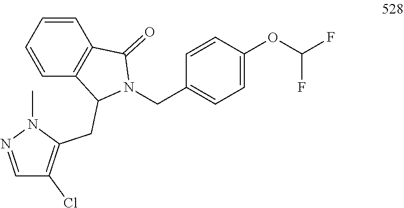 [SMILES: Cn1ncc(Cl)c1CC1c2ccccc2C(=O)N1Cc1ccc(OC(F)F)cc1]

The title compound 528 was prepared according to the procedure described for compound 502 25 as a white solid (4 mg, 4.8%). MS (ESI) m/z 418 [M+H]⁺. ¹H NMR (400 MHz, CDCl₃) δ 7.92 (d, J=7.4 Hz, 1H), 7.52-7.40 (m, 3H), 7.28-7.21 (m, 2H), 7.08 (d, J=8.5 Hz, 2H), 6.64 (d, J=7.6 Hz, 1H), 6.49 (t, J=74 Hz, 1H), 5.38 (d, J=15.2 Hz, 1H), 4.66 (dd, J=8.9, 6.2 Hz, 1H), 4.18 (d, J=15.2 Hz, 1H), 3.45 (s, 3H), 3.37 (dd, J=14.5, 6.2 Hz, 1H), 2.69 (dd, J=14.5, 9.1 Hz, 1H).

### 3-((4-chloro-1-methyl-1H-pyrazol-5-yl)methyl)-2-((5-methoxypyridin-2-yl)methyl)isoindolin-1-one (529)

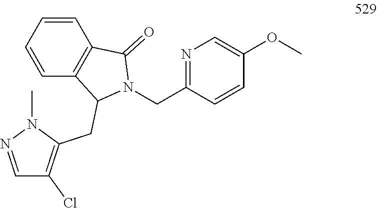 [SMILES: COc1ccc(CN2C(=O)c3ccccc3C2Cc2c(Cl)cnn2C)nc1]

The title compound 529 was prepared according to the procedure described for compound 502 as a white solid (3 mg, 3.5%). MS (ESI) m/z 383 [M+H]⁺. ¹H NMR (400 MHz, CDCl₃) δ 8.24 (d, J=2.8 Hz, 1H), 7.89 (d, J=7.5 Hz, 1H), 7.49-7.43 (m, 2H), 7.39 (t, J=7.0 Hz, 1H), 7.29 (d, J=8.5 Hz, 1H), 7.17 (dd, J=8.6, 2.9 Hz, 1H), 6.55 (d, J=7.5 Hz, 1H), 5.37 (d, J=15.1 Hz, 1H), 4.81 (dd, J=10.1, 5.2 Hz, 1H), 4.49 (d, J=15.0 Hz, 1H), 3.85 (s, 3H), 3.68 (dd, J=14.4, 5.3 Hz, 1H), 3.59 (s, 3H), 2.62 (dd, J=14.3, 10.3 Hz, 1H).

The intermediate 529-2 was prepared as follows:

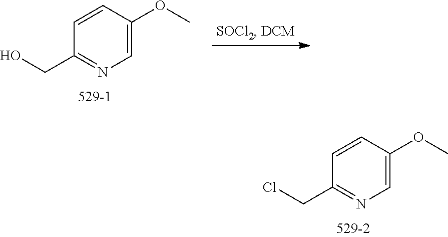

To a solution of (5-methoxypyridin-2-yl)methanol (500 mg, 3.5 mmol) in DCM (5 mL) was added SOCl₂ (826 mg, 7 mmol) at 0 T. The reaction mixture was stirred at 50 TC for 2 h. The solvent was removed and the residue was dissolved in EA and NaHCO₃ (aq.). The organic layer was separated and dried over Na₂SO₄, filtered, and concentrated to give 2-(chloromethyl)-5-methoxypyridine (450 mg, 75%) as an oil. MS (ESI) m/z 158 [M+H]⁺.

### N-((2s,4s)-2-((1-((3-bromopyridin-2-yl)methyl)-3-oxoisoindolin-2-yl)methyl)-5-oxa-7-azaspiro[3.4]oct-6-en-6-yl)cyanamide (530) and N-((2r,4r)-2-((1-((3-bromopyridin-2-yl)methyl)-3-oxoisoindolin-2-yl)methyl)-5-oxa-7-azaspiro[3.4]oct-6-en-6-yl)cyanamide (531)

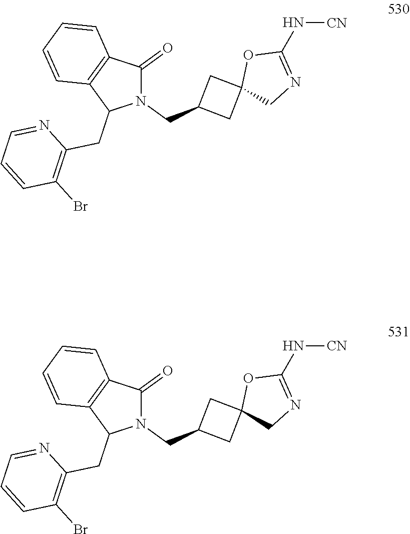 [SMILES: N#CNC1=NC[C@]2(C[C@H](CN3C(=O)c4ccccc4C3Cc3ncccc3Br)C2)O1 ; N#CNC1=NC[C@]2(C[C@@H](CN3C(=O)c4ccccc4C3Cc3ncccc3Br)C2)O1]

The title compounds 530 and 531 were prepared according to the procedure described for compound 520. After prep-HPLC separation, two isomers were obtained. One of the two isomers was a white solid (13 mg, 11.1%), with RT=2.900 min (Waters Sunfire C18 3.5 μm, 50*3.0 mm, 6.5 min 5-95% (0.05% TFA)), MS (ESI) m/z 466 [M+H]⁺. ¹H NMR (400 MHz, DMSO-d₆) δ 8.55 (dd, J=4.8, 1.6 Hz, 1H), 8.04 (dd, J=8.0, 1.6 Hz, 1H), 7.79-7.71 (m, 1H), 7.49-7.45 (m, 2H), 7.26 (dd, J=8.1, 4.7 Hz, 1H), 7.06-6.97 (m, 1H), 5.34 (dd, J=7.4, 5.7 Hz, 1H), 4.04 (dd, J=14.4, 9.8 Hz, 1H), 3.85 (s, 2H), 3.71 (dd, J=14.7, 5.7 Hz, 1H), 3.42 (dd, J=14.4, 6.2 Hz, 1H), 3.26 (dd, J=14.7, 7.5 Hz, 1H), 2.85-2.73 (m, 1H), 2.72-2.66 (m, 1H), 2.61-2.54 (m, 1H), 2.31-2.25 (m, 1H), 2.19-2.13 (m, 1H). The other one of the two isomers was a white solid (14 mg, 12.4%), with RT=2.842 min (Waters Sunfire C18 3.5 μm, 50*3.0 mm, 6.5 min 5-95% (0.05% TFA)), MS (ESI) m/z 466 [M+H]⁺. ¹H NMR (400 MHz, DMSO-d₆) δ 8.57 (dd, J=4.8, 1.6 Hz, 1H), 8.04 (dd, J=8.0, 1.6 Hz, 1H), 7.79-7.70 (m, 1H), 7.52-7.43 (m, 2H), 7.28 (dd, J=8.1, 4.7 Hz, 1H), 7.03-6.98 (m, 11H), 5.35 (dd, J=7.5, 5.7 Hz, 1H), 4.10 (dd, J=14.2, 6.0 Hz, 1H), 3.82 (s, 2H), 3.71 (dd, J=14.7, 5.7 Hz, 11H), 3.41 (dd, J=14.2, 5.6 Hz, 11H), 3.27 (dd, J=14.4, 7.6 Hz, 1H), 2.49-2.34 (m, 5H).

The intermediate 530-4 was prepared as follows:

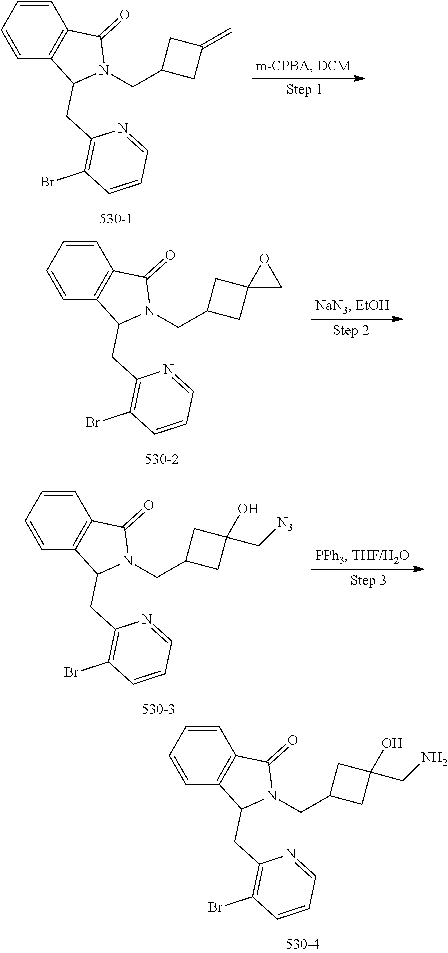 [SMILES: C=C1CC(CN2C(=O)c3ccccc3C2Cc2ncccc2Br)C1 ; O=C1c2ccccc2C(Cc2ncccc2Br)N1CC1CC2(CO2)C1 ; [N-]=[N+]=NCC1(O)CC(CN2C(=O)c3ccccc3C2Cc2ncccc2Br)C1 ; NCC1(O)CC(CN2C(=O)c3ccccc3C2Cc2ncccc2Br)C1]

530-1 was prepared according to the procedure described for the preparation of 222-2.

### Step 1. 2-((1-oxaspiro[2.3]hexan-5-yl)methyl)-3-((3-bromopyridin-2-yl)methyl) isoindolin-1-one (530-2)

A mixture of 3-((3-bromopyridin-2-yl)methyl)-2-((3-methylenecyclobutyl)methyl) isoindolin-1-one (1.5 g, 3.9 mmol) and m-CPBA (1.0 g, 5.1 mmol) in DCM (30 mL) was stirred at r.t. for 16 h. The mixture was quenched with water (30 mL) and extracted with DCM (30 mL*2). The combined organic phase was washed with brine (30 mL), dried over anhydrous Na₂SO₄, filtered and concentrated to give 530-2 (500 mg, 31%) as a colorless oil. MS (ESI) m/z 399 [M+H]⁺.

### Step 2. 2-((3-(azidomethyl)-3-hydroxycyclobutyl)methyl)-3-((3-bromopyridin-2-yl)methyl)isoindolin-1-one (530-3)

To a solution of 530-2 (500 mg, 1.25 mmol) in EtOH/H₂O (10 mL/2 mL) were added NaN₃ (244 mg, 3.75 mmol) and NH₄Cl (210 mg, 3.75 mmol) at r.t. The mixture was stirred at 70° C. overnight. To the mixture was added water (30 mL) and extracted with EA (20 mL*2). The combined organic layers were washed with water (20 mL*2) and brine (20 mL), dried over Na₂SO₄, filtered, purged by N₂ until dryness to give 530-3 (250 mg, 55%) as a yellow oil. MS (ESI) m/z 442 [M+H]⁺.

### Step 3. 2-((3-(aminomethyl)-3-hydroxycyclobutyl)methyl)-3-((3-bromopyridin-2-yl)methyl)isoindolin-1-one (530-4)

A mixture of 530-3 (250 mg, 0.6 mmol) and PPh₃ (330 mg, 1.25 mmol) in THF/H₂O (4 mL/1 mL) was stirred at r.t. overnight. The mixture was concentrated and the residue was purified by prep-HPLC to give 530-4 (200 mg, 90%) as a white solid. MS (ESI) m/z 416 [M+H]⁺. 3-((4-chloro-1-methyl-1H-pyrazol-5-yl)methyl)-2-((5-fluoro-6-methoxypyridin-3-yl)methyl)isoindolin-1-one (532)

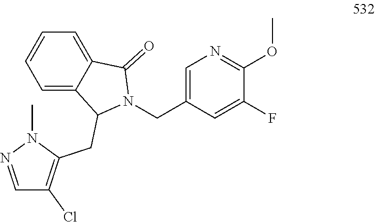 [SMILES: COc1ncc(CN2C(=O)c3ccccc3C2Cc2c(Cl)cnn2C)cc1F]

The title compound 532 was prepared according to the procedure described for compound 502 as a white solid (59 mg, 11.1%). MS (ESI) m/z 401 [M+H]⁺. ¹H NMR (400 MHz, DMSO-d₆) δ 7.91 (s, 1H), 7.76-7.69 (m, 1H), 7.57-7.46 (m, 4H), 6.83 (d, J=6.4 Hz, 1H), 5.11 (d, J=15.2 Hz, 1H), 4.80-4.72 (m, 1H), 4.38 (d, J=15.2 Hz, 1H), 3.92 (s, 3H), 3.60 (s, 3H), 3.53 (dd, J=14.8, 5.8 Hz, 1H), 2.98 (dd, J=14.8, 8.5 Hz, 1H).

The intermediate 532-3 was prepared as follows:

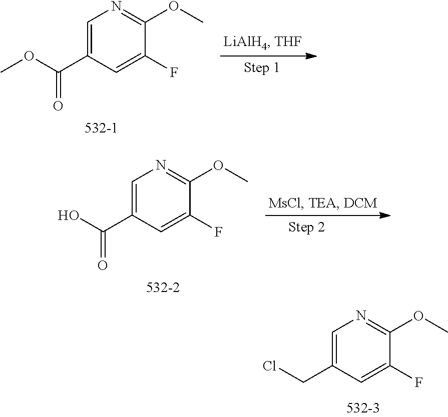

Step 1 was performed according to the procedure outlined for preparation of 263-2.

### Step 2. 5-(chloromethyl)-3-fluoro-2-methoxypyridine (532-3)

To a solution of (5-fluoro-6-methoxypyridin-3-yl)methanol (250 mg, 1.59 mmol) and TEA (225 mg, 1.75 mmol) in DCM (5 mL) was added MsCl (200 mg 1.75 mmol) under N₂. The mixture was stirred at r.t. for 1h. Water (10 mL) was added and the resulting mixture was extracted with EA (10 mL*3). The combined organic layers were washed with brine (20 mL), dried over Na₂SO₄, filtered and concentrated. The residue was purified by FCC (PE/EA=5/1) to give 532-3 (240 mg, 86%) as a yellow oil. MS (ESI) m/z 176 [M+H]⁺.

### (2s,4S)-2-(((R)-1-((3,4-dichloro-1-methyl-1H-pyrazol-5-yl)methyl)-3-oxoisoindolin-2-yl)methyl)-5-oxa-7-azaspiro[3.4]octan-6-one (533) and (2s,4R)-2-(((S)-1-((3,4-dichloro-1-methyl-1H-pyrazol-5-yl)methyl)-3-oxoisoindolin-2-yl)methyl)-5-oxa-7-azaspiro[3.4]octan-6-one (534)

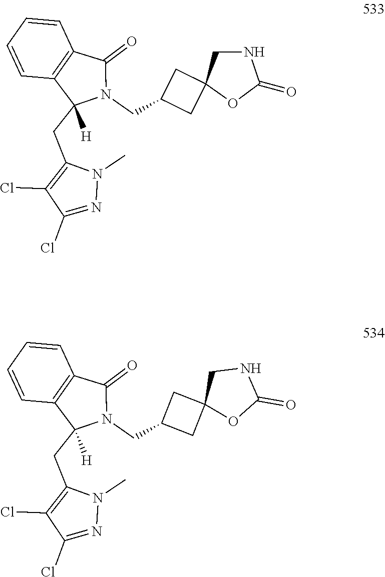 [SMILES: Cn1nc(Cl)c(Cl)c1C[C@@H]1c2ccccc2C(=O)N1C[C@H]1C[C@]2(CNC(=O)O2)C1 ; Cn1nc(Cl)c(Cl)c1C[C@H]1c2ccccc2C(=O)N1C[C@H]1C[C@]2(CNC(=O)O2)C1]

Chiral separation of compound 499 by chiral prep-HPLC (column: CHIRALPAK IA; column size: 5 cm×25 cm, 5 μm; mobile phase A: Hex; mobile phase B: EtOH) was conducted to afford two enantiomers: compound 533 and compound 534. One of the two enantiomers was a white solid, with RT=1.313 min (CHIRAL HPLC, column: CHIRALPAK IA-3; column size: 4.6*50 mm, 3 μm; mobile phase: Hex (0.1% DEA):EtOH=50:50; flow: 25.0 mL/min). MS (ESI) m/z 435 [M+H]⁺. ¹H NMR (400 MHz, CDCl₃) δ 7.85 (d, J=7.3 Hz, 1H), 7.53-7.40 (m, 2H), 6.59 (d, J=7.4 Hz, 1H), 5.75 (s, 1H), 4.81 (dd, J=10.0, 5.3 Hz, 1H), 4.21 (dd, J=14.4, 5.3 Hz, 1H), 3.61 (s, 2H), 3.50 (dd, J=14.4, 5.3 Hz, 1H), 3.40 (s, 3H), 3.24 (dd, J=14.6, 6.1 Hz, 1H), 2.55 (dd, J=14.4, 10.0 Hz, 1H), 2.49-2.26 (m, 5H). The other one of the two enantiomers was a white solid, with RT=1.708 min, (CHIRAL HPLC, column: CHIRALPAK IA-3; column size: 4.6*50 mm, 3 μm; mobile phase: Hex (0.1% DEA): EtOH=50:50; flow: 25.0 mL/min). MS (ESI) m/z 435 [M+H]⁺. ¹H NMR (400 MHz, CDCl₃) δ 7.85 (d, J=7.3 Hz, 1H), 7.53-7.40 (m, 2H), 6.59 (d, J=7.4 Hz, 1H), 5.74 (s, 1H), 4.81 (dd, J=10.0, 5.2 Hz, 1H), 4.21 (dd, J=14.4, 5.3 Hz, 1H), 3.61 (s, 2H), 3.50 (dd, J=14.4, 5.3 Hz, 1H), 3.40 (s, 3H), 3.24 (dd, J=14.7, 6.1 Hz, 1H), 2.55 (dd, J=14.4, 10.0 Hz, 1H), 2.49-2.28 (m, 5H).

### 2-((1-((4-chloro-1-methyl-1H-pyrazol-5-yl)methyl)-3-oxoisoindolin-2-yl)methyl)-5-oxa-7-azaspiro[3.4]octan-6-one-8,8-d₂ (535)

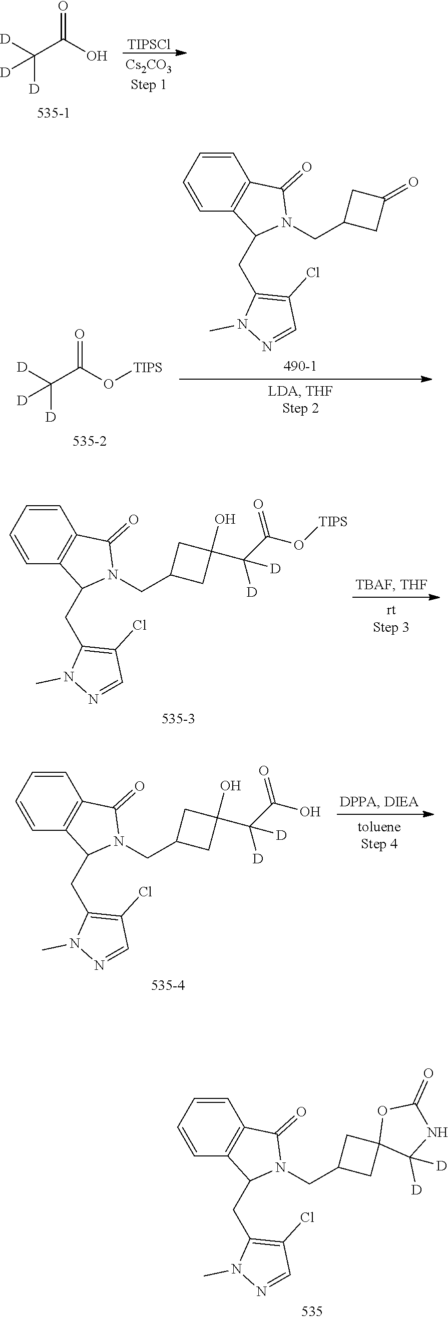

### Step 1. triisopropylsilyl acetate-d₃ (535-2)

To a solution of acetic-2,2,2-d₃ acid (1.1 g, 17.5 mmol) in DCM (100 mL) was added Cs₂CO₃ (5.7 g, 17.5 mmol) in DCM (100 mL) at r.t., then was added TIPSCl (3.4 g, 17.5 mol) at r.t. The reaction mixture was stirred at r.t. overnight. The mixture was filtered and concentrated to give crude product 535-2 (3.5 g, 92%).

### Step 2. triisopropylsilyl 2-(3-((1-((4-chloro-1-methyl-1H-pyrazol-5-yl)methyl)-3-oxoisoindolin-2-yl)methyl)-1-hydroxycyclobutyl)acetate-d₂ (535-3)

To a solution of LDA (0.40 mL, 2M in THF, 0.80 mmol) in THF (1 mL) was added a solution of 535-2 (179 mg, 0.82 mmol) in THF (1 mL) at −78° C. under N₂. The reaction mixture was stirred for 0.5h at −78° C. under N₂. Then a solution of 490-1 (100 mg, 0.29 mmol) in THF (1 mL) was added and stirred for 1 h under N₂. The reaction mixture was quenched with NH₄Cl (aq.) (10 mL), extracted with EA (5 mL*3). The combined organic layers were washed with brine (5 mL), dried over Na₂SO₄, filtered and concentrated. The residue was purified by FCC (PE/EA=1/1) to give 535-3 (100 mg, 61%) as a yellow oil. MS (ESI) m/z 562 [M+H]⁺.

### Step 3. 2-(3-((1-((4-chloro-1-methyl-1H-pyrazol-5-yl)methyl)-3-oxoisoindolin-2-yl)methyl)-1-hydroxycyclobutyl)acetic-2,2-d₂ acid (535-4)

To a solution of 535-3 (100 mg, 0.18 mmol) in THF (3 mL) was added TBAF (0.3 mL, 1M in THF, 0.3 mmol) at ice-bath. The reaction mixture was stirred at r.t. overnight under N₂. The mixture was poured into water (10 mL) and acidified to pH 5-6, extracted with DCM (10 mL*3). The combined organic layers were washed with brine (20 mL), dried over Na₂SO₄, filtered and concentrated to give 535-4 (72 mg, 100%) as a yellow oil. MS (ESI) m/z 406 [M+H]⁺.

### Step 4. 2-((1-((4-chloro-1-methyl-IH-pyrazol-5-yl)methyl)-3-oxoisoindolin-2-yl)methyl)-5-oxa-7-azaspiro[3.4]octan-6-one-8,8-d₂ (535)

A mixture of 535-4 (72 mg, 0.18 mmol), DPPA (147 mg, 0.54 mmol) and DIEA (232 mg, 1.8 mmol) in toluene (2 mL) was refluxed for 2 h under N₂. The mixture was cooled to r.t. and concentrated. The residue was purified by prep-HPLC to give 535 (18 mg, 25%) as a white solid. MS (ESI) m/z 403 [M+H]⁺. ¹H NMR (400 MHz, DMSO-d₆) δ 7.71-7.64 (m, 1H), 7.56 (s, 1H), 7.54-7.44 (m, 3H), 6.81-6.75 (m, 1H), 4.88 (dd, J=8.6, 5.6 Hz, 1H), 4.00 (dd, J=14.2, 6.1 Hz, 1H), 3.64 (s, 3H), 3.56 (dd, J=14.8, 5.7 Hz, 1H), 3.36-3.34 (m, 1H), 2.87 (dd, J=14.8, 8.7 Hz, 1H), 2.39-2.36 (m, 1H), 2.29-2.19 (m, 3H), 2.11-2.08 (m, 1H).

### (2s,4S)-2-(((R)-1-((4-chloro-1-methyl-1H-pyrazol-5-yl)methyl)-3-oxoisoindolin-2-yl)methyl)-5-oxa-7-azaspiro[3.4]octan-6-one-8,8-d₂ (536) and (2s,4R)-2-(((S)-1-((4-chloro-1-methyl-1H-pyrazol-5-yl)methyl)-3-oxoisoindolin-2-yl)methyl)-5-oxa-7-azaspiro[3.4]octan-6-one-8,8-d₂ (537)

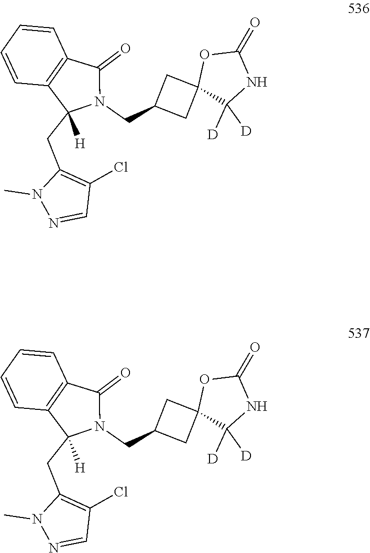 [SMILES: [2H]C1([2H])NC(=O)O[C@]12C[C@@H](CN1C(=O)c3ccccc3[C@H]1Cc1c(Cl)cnn1C)C2 ; [2H]C1([2H])NC(=O)O[C@]12C[C@@H](CN1C(=O)c3ccccc3[C@@H]1Cc1c(Cl)cnn1C)C2]

Chiral separation of 535 by chiral prep-HPLC (column: CHIRALPAK IA; column size: 2 cm×25 cm, 5 μm; mobile phase A: MTBE; mobile phase B: EtOH) was conducted to afford two enantiomers: compound 536 and compound 537. One of the two enantiomers was a white solid, with RT=2.013 min (CHIRAL HPLC, column: CHIRALPAK IA-3; column size: 4.6*50 mm, 3 μm; mobile phase: MTBE (0.1% DEA):EtOH=80:20; flow: 1.0 mL/min). MS (ESI) m/z 425 [M+Na]+. ¹H NMR (400 MHz, CD₃OD) δ 7.81-7.74 (m, 1H), 7.52-7.50 (m, 3H), 6.81-6.74 (m, 1H), 5.00 (dd, J=8.9, 5.7 Hz, 1H), 4.16 (dd, J=14.1, 5.2 Hz, 1H), 3.64-3.58 (m, 4H), 3.47-3.37 (m, 1H), 2.89 (dd, J=14.7, 8.8 Hz, 1H), 2.50-2.47 (m, 1H), 2.42-2.26 (m, 4H). The other one of the two enantiomers was a white solid, with RT=2.643 min (CHIRAL HPLC, column: CHIRALPAK IA-3; column size: 4.6*50 mm, 3 μm; mobile phase: MTBE (0.1% DEA): EtOH=80:20; flow: 1.0 mL/min). MS (ESI) m/z 425 [M+Na]+. 1H NMR (400 MHz, CD₃OD) δ 7.81-7.73 (m, 1H), 7.56-7.45 (m, 3H), 6.81-6.73 (m, 1H), 5.00 (dd, J=8.9, 5.7 Hz, 1H), 4.16 (dd, J=14.0, 5.5 Hz, 1H), 3.72-3.56 (m, 4H), 3.42 (dd, J=14.1, 5.1 Hz, 1H), 2.89 (dd, J=14.8, 9.0 Hz, 1H), 2.50-2.45 (m, 1H), 2.42-2.18 (m, 4H).

### 2-((1-((5-chloro-1,2,3-thiadiazol-4-yl)methyl)-3-oxoisoindolin-2-yl)methyl)-5-oxa-7-azaspiro[3.4]octan-6-one (538)

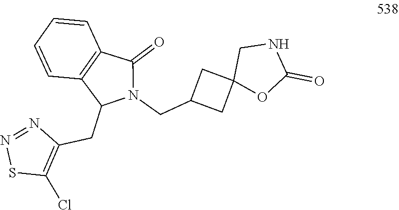 [SMILES: O=C1NCC2(CC(CN3C(=O)c4ccccc4C3Cc3nnsc3Cl)C2)O1]

The title compound 538 was prepared according to the procedure described for compound 307 as a white solid (5 mg, 13.3%). MS (ESI) m/z 405 [M+H]⁺. ¹H NMR (400 MHz, CDCl₃): δ 7.83-7.71 (m, 1H), 7.49-7.42 (m, 2H), 7.10-7.01 (m, 1H), 5.13 (dd, J=7.4, 4.4 Hz, 1H), 4.85 (s, 1H), 4.28-4.16 (m, 11H), 3.68 (dd, J=14.8, 4.4 Hz, 1H), 3.61 (s, 2H), 3.35 (dd, J=14.8, 6.8 Hz, 2H), 2.47-2.32 (m, 5H).

The intermediate 538-3 was prepared as follows:

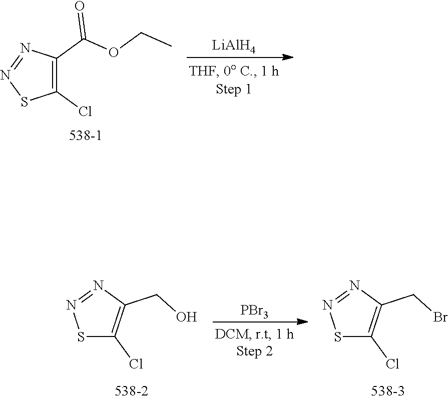

Step 1 was performed according to the procedure outlined for the preparation of 217-2.

Step 2 was performed according to the procedure outlined for the preparation of 217-3.

### 2-((3′-((4-chloro-1-methyl-1H-pyrazol-5-yl)methyl)spiro[cyclopropane-1,1′-isoindolin]-2′-yl)methyl)-5-oxa-7-azaspiro[3.4]octan-6-one (539)

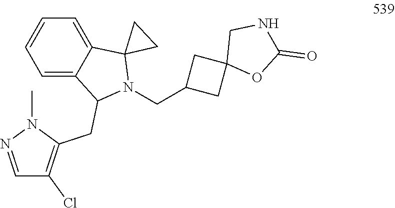 [SMILES: Cn1ncc(Cl)c1CC1c2ccccc2C2(CC2)N1CC1CC2(CNC(=O)O2)C1]

The title compound 539 was prepared according to the procedure described for compound 270 as a light yellow solid (10 mg, 2.5%). MS (ESI) m/z 413 [M+H]⁺. ¹H NMR (400 MHz, CDCl₃) δ 7.46 (s, 1H), 7.42 (t, J=7.6 Hz, 1H), 7.22 (t, J=7.6 Hz, 1H), 6.91 (d, J=7.8 Hz, 1H), 6.37 (d, J=7.5 Hz, 1H), 4.90 (s, 1H), 4.80 (dd, J=11.6, 5.1 Hz, 1H), 3.52 (dd, J=18.0, 4.8 Hz, 1H), 3.61 (s, 2H), 3.52 (dd, J=17.4, 12.4 Hz, 1H), 3.22 (s, 3H), 3.19-3.08 (m, 2H), 2.62-2.39 (m, 4H), 2.32-2.22 (m, 2H), 2.00-1.88 (m, 1H), 1.64-1.53 (m, 1H), 1.16-1.08 (m, 1H).

The intermediate 539-3 was prepared as follows:

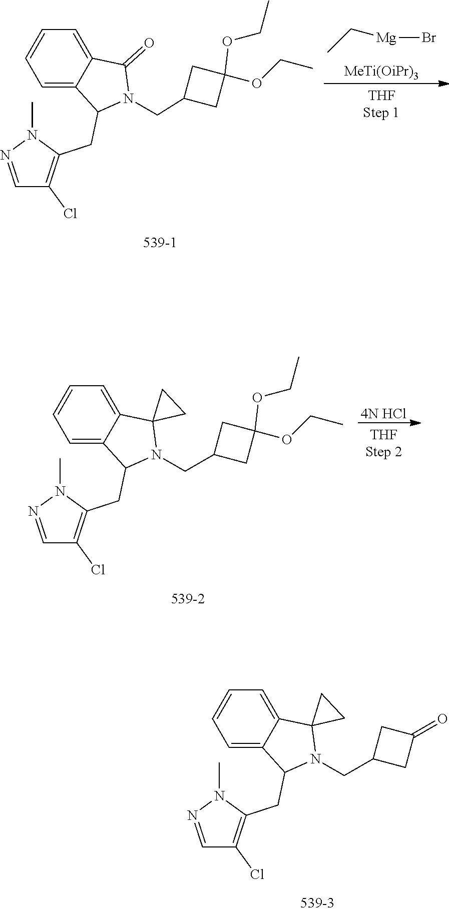

539-1 was prepared according to the procedure outlined for the preparation of 270-2.

### Step 1. 3′-((4-chloro-1-methyl-1H-pyrazol-5-yl)methyl)-2′-((3,3-diethoxycyclobutyl)methyl)spiro[cyclopropane-1,1′-isoindoline](539-2)

To a solution of 539-1 (2.0 g, 4.8 mmol) and triisopropoxy(methyl)titanium (2.3 g, 9.6 mmol) in THF (30 mL) was added ethylmagnesium bromide (19.2 mL, 19.2 mmol) at 0° C. The mixture was stirred at r.t. for 3 h, then quenched with water, extracted with EA and concentrated in vacuo. The residue was purified by silica gel flash column chromatography (PE/EA=1/2) to afford 539-2 (600 mg, 29.1%) as a yellow oil. MS (ESI) m/z 430 [M+H]⁺.

### Step 2. 3-((3′-((4-chloro-1-methyl-1H-pyrazol-5-yl)methyl)spiro[cyclopropane-1,1′-isoindolin]-2′-yl)methyl)cyclobutan-1-one (539-3)

To a solution of 539-2 (600 mg, 1.4 mmol) in THF (8 mL) was added 4N HCl (2 mL) and the reaction mixture was stirred at 50° C. for 2 h. The reaction mixture was diluted with EA and washed with water. The organic layer was separated, dried over anhydrous Na₂SO₄ and concentrated in vacuo to afford 539-3 (400 mg) as a yellow oil, which was used for the next step without further purification. MS (ESI) m/z 356 [M+H]⁺.

### 2-((3-((4-chloro-1-methyl-1H-pyrazol-5-yl)methyl)-1,1-dioxidobenzo[d]isothiazol-2(3H)-yl)methyl)-5-oxa-7-azaspiro[3.4]octan-6-one (540)

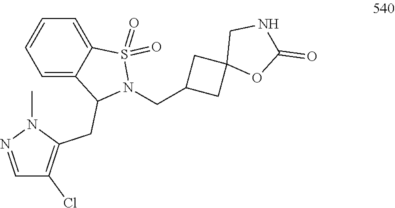 [SMILES: Cn1ncc(Cl)c1CC1c2ccccc2S(=O)(=O)N1CC1CC2(CNC(=O)O2)C1]

The title compound 540 was prepared according to the procedure described for compound 270 as a white solid (16 mg, 16%). MS (ESI) m/z 437 [M+H]⁺. ¹H NMR (400 MHz, DMSO-d₆) δ 7.89 (d, J=7.3 Hz, 1H), 7.71-7.59 (m, 2H), 7.55 (s, 1H), 7.52 (s, 1H), 7.08 (d, J=7.4 Hz, 1H), 4.87 (t, J=7.2 Hz, 1H), 3.73 (s, 3H), 3.50 (s, 2H), 3.39-3.31 (m, 2H), 3.19 (dd, J=14.2, 5.3 Hz, 1H), 3.08 (dd, J=1 4.9, 7.1 Hz, 1H), 2.33-2.23 (m, 2H), 2.11-1.98 (m, 3H).

The Intermediate 540-6 was prepared as follows:

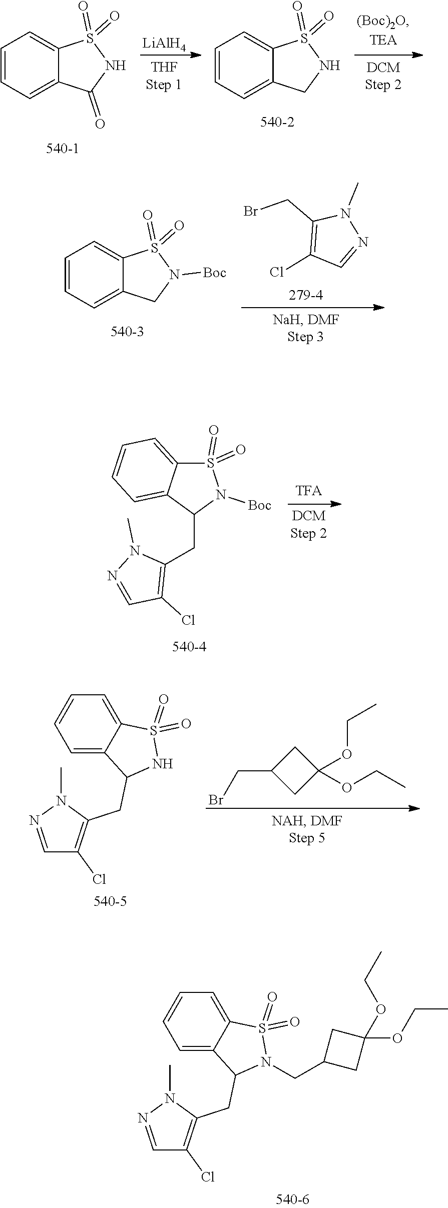

Step 1 was performed according to the procedure outlined for the preparation of 217-2.

Step 2 was performed according to the procedure outlined for the preparation of Int 12-1.

### Step 3. tert-butyl 3-((4-chloro-1-methyl-1H-pyrazol-5-yl)methyl)benzo[d]isothiazole-2(3H)-carboxylate 1,1-dioxide (540-4)

To a solution of 540-3 (1.3 g, 4.83 mmol) in DMF (5 mL) was added NaH (213 mg, 5.32 mmol) at 0° C. The mixture was stirred at 0° C. for 30 min. Then 279-4 (1.1 g, 5.32 mmol) was added to the mixture. The reaction mixture was stirred at 65° C. for 2 h, then quenched with water, extracted with EA and concentrated in vacuo. The residue was purified by silica gel flash column chromatography (PE/EA=1/2) to afford 540-4 (450 mg, 23.5%) as a yellow oil. MS (ESI) m/z 398 [M+H]⁺.

Step 4 was performed according to the procedure outlined for step 3 for the preparation of Int 12.

Step 5 was performed according to the procedure outlined for the preparation of 270-2.

### 2-((3-((4-chloro-1-methyl-1H-pyrazol-5-yl)methyl)-5-(difluoromethoxy)-1-oxoisoindolin-2-yl)methyl)-5-oxa-7-azaspiro[3.4]octan-6-one (541)

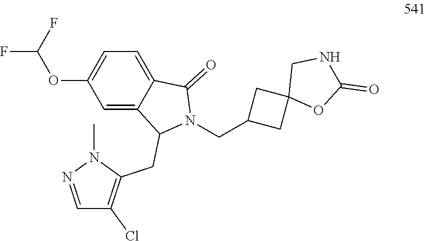 [SMILES: Cn1ncc(Cl)c1CC1c2cc(OC(F)F)ccc2C(=O)N1CC1CC2(CNC(=O)O2)C1]

The title compound 541 was prepared according to the procedure described for compound 270 using 421-2 as a starting material to afford a white solid (20 mg, 34%). MS (ESI) m/z 467 [M+H]⁺. ¹H NMR (400 MHz, CDCl₃) δ 7.85 (d, J=8.3 Hz, 1H), 7.54 (s, 1H), 7.24 (dd, J=8.3, 2.0 Hz, 1H), 6.48 (t, J=72.8 Hz, 1H), 6.27 (d, J=2.1 Hz, 1H), 4.92 (s, 1H), 4.83 (dd, J=9.7, 5.6 Hz, 1H), 4.20 (dd, J=14.4, 5.3 Hz, 1H), 3.62 (s, 2H), 3.52 (s, 3H), 3.53-3.46 (m, 1H), 3.20 (dd, J=14.4, 6.6 Hz, 1H), 2.62 (dd, J=14.4, 9.8 Hz, 1H), 2.48-2.36 (m, 5H).

### 3-((4-chloro-1-methyl-1H-pyrazol-5-yl)methyl)-2-(4-(methylthio)benzyl)isoindolin-1-one

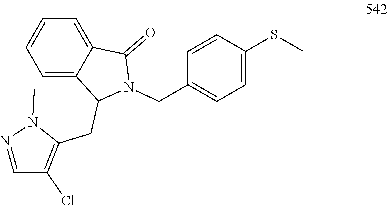 [SMILES: CSc1ccc(CN2C(=O)c3ccccc3C2Cc2c(Cl)cnn2C)cc1]

The title compound 542 was prepared according to the procedure described for 502 as a white solid (25 mg, 11%). MS (ESI) m/z 398 [M+H]⁺. ¹H NMR (400 MHz, DMSO-d₆) δ 7.77-7.70 (m, 1H), 7.55 (s, 1H), 7.54-7.49 (m, 2H), 7.22 (d, J=8.0 Hz, 2H), 7.14 (d, J=8.0 Hz, 2H), 6.91-6.85 (m, 1H), 5.17 (d, J=15.2 Hz, 1H), 4.63 (dd, J=8.0, 6.0 Hz, 1H), 4.24 (d, J=15.2 Hz, 1H), 3.57 (s, 3H), 3.47 (dd, J=14.8, 6.1 Hz, 1H), 2.99 (dd, J=14.8, 8.0 Hz, 1H), 2.44 (s, 3H).

### 3-((4-chloro-1-methyl-1H-pyrazol-5-yl)methyl)-2-((6-fluoro-5-methoxypyridin-2-yl)methyl)isoindolin-1-one (543)

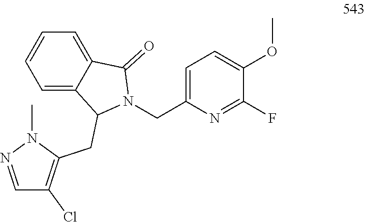 [SMILES: COc1ccc(CN2C(=O)c3ccccc3C2Cc2c(Cl)cnn2C)nc1F]

The title compound 543 was prepared according to the procedure described for 502 as a white solid (35 mg, 21.8%). MS (ESI) m/z 401 [M+H]⁺. ¹H NMR (400 MHz, DMSO-d₆) δ 7.74-7.70 (m, 1H), 7.65-7.59 (m, 1H), 7.58-7.47 (m, 3H), 7.20 (d, J=8.0 Hz, 1H), 6.93-6.86 (m, 1H), 5.11 (d, J=15.6 Hz, 1H), 4.85 (dd, J=8.4, 5.8 Hz, 1H), 4.34 (d, J=15.6 Hz, 1H), 3.85 (s, 3H), 3.63 (s, 3H), 3.51 (dd, J=14.8, 6.0 Hz, 1H), 2.99 (dd, J=14.8, 8.4 Hz, 1H).

### 3-((4-chloro-1-methyl-1H-pyrazol-5-yl)methyl)-2-(3-chloro-4-methoxybenzyl)isoindolin-1-one (544)

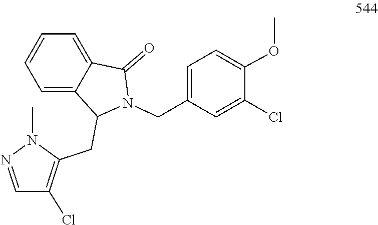 [SMILES: COc1ccc(CN2C(=O)c3ccccc3C2Cc2c(Cl)cnn2C)cc1Cl]

The title compound 544 was prepared according to the procedure described for 502 as a white solid (60 mg, 25.2%). MS (ESI) m/z 416 [M+H]⁺. ¹H NMR (400 MHz, DMSO-d₆) δ 7.77-7.70 (m, 1H), 7.55 (s, 1H), 7.55-7.47 (m, 2H), 7.30 (d, J=2.0 Hz, 1H), 7.19-7.07 (m, 2H), 6.88-6.82 (m, 1H), 5.14 (d, J=15.2 Hz, 1H), 4.64 (dd, J=8.0, 6.0 Hz, 1H), 4.25 (d, J=15.2 Hz, 1H), 3.82 (s, 3H), 3.59 (s, 3H), 3.52 (dd, J=14.8, 6.0 Hz, 1H), 2.98 (dd, J=14.8, 8.4 Hz, 1H).

### 6-methyl-5-(((R)-3-oxo-2-(((2s,4S)-6-oxo-5-oxa-7-azaspiro[3.4]octan-2-yl)methyl)isoindolin-1-yl)methyl)pyrimidine-4-carbonitrile (545) and 6-methyl-5-(((S)-3-oxo-2-(((2s,4R)-6-oxo-5-oxa-7-azaspiro[3.4]octan-2-yl)methyl)isoindolin-1-yl)methyl)pyrimidine-4-carbonitrile (546)

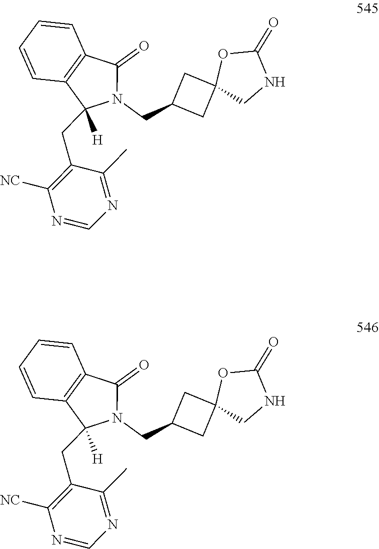 [SMILES: Cc1ncnc(C#N)c1C[C@@H]1c2ccccc2C(=O)N1C[C@H]1C[C@]2(CNC(=O)O2)C1 ; Cc1ncnc(C#N)c1C[C@H]1c2ccccc2C(=O)N1C[C@H]1C[C@]2(CNC(=O)O2)C1]

Chiral separation of compound 491 by chiral prep-HPLC (IA, 4.6 mm*250 mm 5 um, Hex/EtOH=30/70, 1 mL/min) was conducted to afford two enantiomers: compound 545 and compound 546. One of the two enantiomers was a white solid, with RT=6.524 min (IA, 4.6 mm*250 mm 5 μm, Hex/EtOH=30/70, 1 mL/min). MS (ESI) m/z 404 [M+H]⁺. ¹H NMR (400 MHz, DMSO-d₆) δ 9.20 (s, 1H), 7.73 (d, J=7.6 Hz, 1H), 7.52-7.43 (m, 3H), 6.53 (d, J=7.6 Hz, 1H), 4.98 (dd, J=10.0, 5.6 Hz, 1H), 4.03 (dd, J=13.6, 5.6 Hz, 1H), 3.89 (dd, J=14.0, 5.2 Hz, 1H), 3.51 (s, 2H), 3.44 (dd, J=14.4, 5.1 Hz, 1H), 2.70 (dd, J=13.6, 10.0 Hz, 1H), 2.57 (s, 3H), 2.41-2.25 (m, 4H), 2.13-2.11 (m, 1H). The other one of the two enantiomers was a white solid, with RT=9.713 min, (IA, 4.6 mm*250 mm 5 um, Hex/EtOH=30/70, 1 mL/min). MS (ESI) m/z 404 [M+H]⁺. ¹H NMR (400 MHz, DMSO-d₆) δ 9.20 (s, 1H), 7.73 (d, J=7.6 Hz, 1H), 7.52-7.43 (m, 3H), 6.54 (d, J=7.6 Hz, 1H), 4.98 (dd, J=10.0, 5.6 Hz, 1H), 4.03 (dd, J=13.6, 5.6 Hz, 1H), 3.89 (dd, J=14.0, 5.2 Hz, 1H), 3.51 (s, 2H), 3.44 (dd, J=14.2, 5.3 Hz, 1H), 2.70 (dd, J=13.6, 10.0 Hz, 1H), 2.57 (s, 3H), 2.41-2.25 (m, 4H), 2.13-2.11 (m, 1H).

### 2-((1-((4-chloro-3-fluoro-1-methyl-1H-pyrazol-5-yl)methyl)-3-oxoisoindolin-2-yl)methyl)-5-oxa-7-azaspiro[3.4]octan-6-one (547)

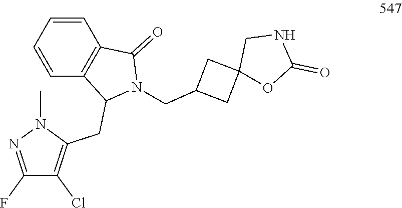 [SMILES: Cn1nc(F)c(Cl)c1CC1c2ccccc2C(=O)N1CC1CC2(CNC(=O)O2)C1]

The title compound 547 was prepared according to the procedure described for 451 as a white solid (55 mg, 32.5%). MS (ESI) m/z 419 [M+H]⁺. ¹H NMR (400 MHz, CDCl₃) δ 7.86 (dd, J=7.4, 2.7 Hz, 1H), 7.53-7.40 (m, 2H), 6.68-6.61 (m, 1H), 5.50 (s, 1H), 4.84-4.79 (m, 1H), 4.25-4.19 (m, 1H), 3.61 (s, 2H), 3.51-3.44 (m, 1H), 3.33 (s, 3H), 3.26-3.20 (m, 1H), 2.57-2.50 (m, 1H), 2.48-2.30 (m, 5H).

The intermediate 547-4 was prepared as follows:

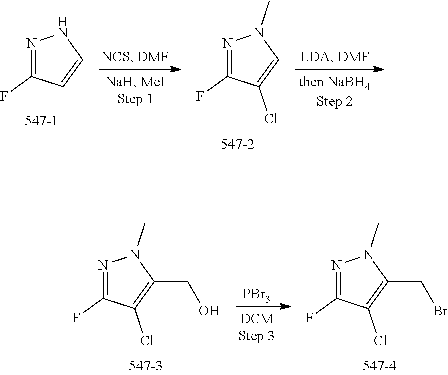

### Step 1. 4-chloro-3-fluoro-1-methyl-pyrazole (547-2)

A mixture of 3-fluoro-1H-pyrazole (500 mg, 5.81 mmol) and NCS (814 mg, 6.1 mmol) in DMF (20 mL) was stirred at 70° C. for 3 h. The mixture was cooled to r.t. and followed by the addition of NaH (511 mg, 12.78 mmol) and Mel (3.29 g, 23.24 mmol). The mixture was stirred for 2 h, then quenched with water (10 mL), and extracted with EA (10 mL*3). The organics were combined and dried (Na₂SO₄) before concentration to dryness. The residue was purified by flash (PE/EA=10/1) to afford 547-2 (561 mg, 71.8%) as a colorless oil. MS (ESI) m/z 135 [M+H]⁺.

### Step 2. (4-chloro-5-fluoro-2-methyl-pyrazol-3-yl)methanol (547-3)

To a solution of 547-2 (300 mg, 2.23 mmol) in THF (10 mL) was added LDA (1.7 mL, 3.34 mmol). The mixture was stirred in a dry ice/acetone bath for 30 min, followed by the addition of DMF (345 μL, 4.46 mmol) in THF (2 mL) and stirred for 1 h. The mixture was then warmed to r.t. and followed by the addition of NaBH₄ (254 mg, 6.69 mmol). The mixture was stirred for 1 h, quenched with sat. NH₄Cl (aq.) (20 mL), and extracted with EA (2*30 mL). The organics were combined and dried (Na₂SO₄) before concentration to dryness. The residue was purified by flash (PE/EA=4/1) to afford 547-3 (254 mg, 69%) as a colorless oil. MS (ESI) m/z 165 [M+H]⁺.

Step 3 was performed according to the procedure outlined for the preparation of 217-3.

### trans 2-((2-(1H-1,2,3-triazol-4-yl)cyclopropyl)methyl)-3-((4-chloro-1-methyl-1H-pyrazol-5-yl)methyl)isoindolin-1-one (548) and cis 2-((2-(1H-1,2,3-triazol-4-yl)cyclopropyl)methyl)-3-((4-chloro-1-methyl-1H-pyrazol-5-yl)methyl)isoindolin-1-one (604)

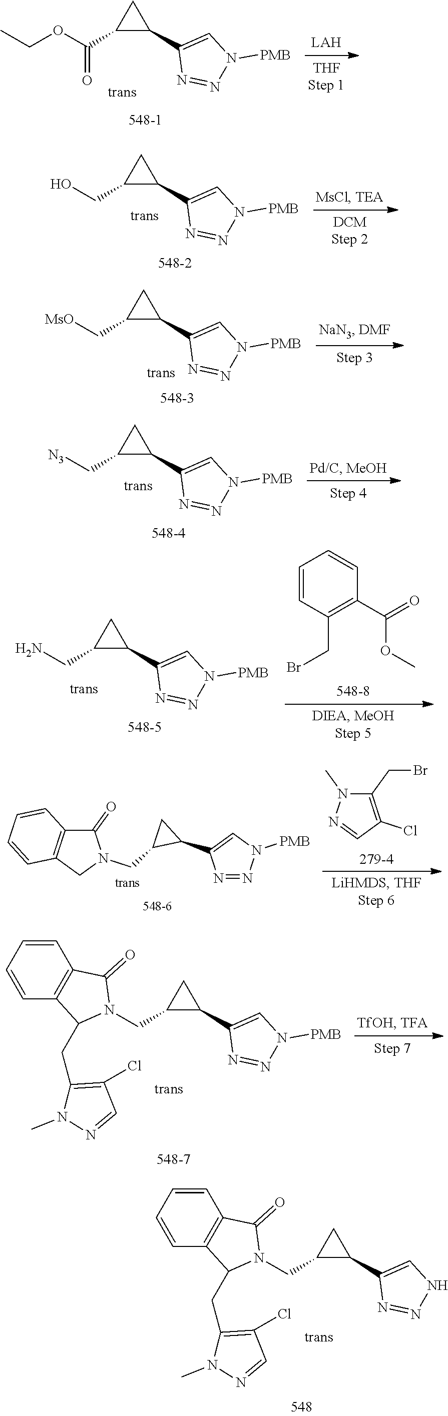

Step 1 was performed according to the procedure outlined for the preparation of 279-3.

Step 2 was performed according to the procedure outlined for the preparation of 132-2.

### Step 3. Trans 4-(2-(azidomethyl)cyclopropyl)-1-(4-methoxybenzyl)-1H-1,2,3-triazole (548-4)

To a solution of 548-3 (585 mg, 1.73 mmol) in DMF (10 mL) was added NaN₃ (135 mg, 2.08 mmol) at r.t. The mixture was stirred at 65° C. for 2 h. After filtration, water (30 mL) was added to the mixture and the mixture was extracted with EA (30 mL*3). The combined organic layers were separated, dried over anhydrous Na₂SO₄, filtered and concentrated under vacuum. The residue was purified by FCC (PE/EA=3/1) to give 548-4 (290 mg, 59%) as a colorless liquid. MS (ESI) m/z 285 [M+H]⁺.

### Step 4. Trans (2-(1-(4-methoxybenzyl)-1H-1,2,3-triazol-4-yl)cyclopropyl)methanamine (548-5)

To a solution of 548-4 (290 mg, 1.02 mmol) in MeOH (10 mL) was added Pd/C (87 mg) at r.t. The mixture was stirred at r.t. for 8 h under 1 atm H₂. The mixture was filtered, and the filtrate was concentrated to give 548-5 (258 mg, crude). MS (ESI) m/z 259 [M+H]⁺.

### Step 5. Trans 2-((2-(1-(4-methoxybenzyl)-1H-1,2,3-triazol-4-yl)cyclopropyl)methyl)isoindolin-1-one (548-6)

To a solution of 548-5 (258 mg, 0.96 mmol) and methyl 2-(bromomethyl)benzoate (200 mg, 0.90 mmol) in MeOH (10 mL) was added DIEA (0.4 mL, 2.42 mmol) at r.t. The mixture was stirred at 60′C for 16 h. The mixture was concentrated and purified by FCC (PE/EA=1/1) to give 548-6 (200 mg, 53%) as a white solid. MS (ESI) m/z 375 [M+H]⁺.

Step 6 was performed according to the procedure outlined for the preparation of Int 12-2.

Step 7 was performed according to the procedure outlined for step 2 for the preparation of 4 to give 548 (3 mg, 7.5%) as an off-white solid. MS (ESI) m/z 383 [M+H]⁺. ¹H NMR (400 MHz, DMSO-d₆) δ 7.72-7.66 (m, 1H), 7.60-7.45 (m, 4H), 6.84-6.75 (m, 1H), 5.05 (dd, J=8.4, 5.8 Hz, 1H), 4.09-3.94 (m, 1H), 3.69-3.55 (m, 4H), 3.29-3.17 (m, 1H), 2.91 (dd, J=14.8, 8.6 Hz, 1H), 2.18-1.87 (m, 1H), 1.48-1.36 (m, 1H), 1.20-0.99 (m, 1H), 0.96-0.90 (m, 1H).

The intermediate 548-1 was prepared as follows:

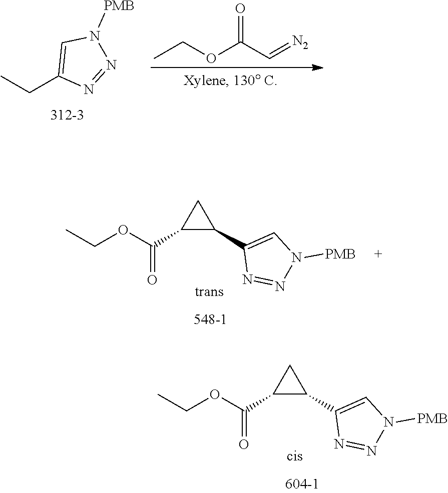

The mixture of 312-3 (3.4 g, 15.8 mmol) and ethyl 2-diazoacetate (5.4 g, 47.44 mmol) in xylene (30 mL) was stirred at 130° C. for 16 h. The mixture was directly purified by FCC (PE/EA=2/1) to give 548-1 (2.1 g, 44%) as a light-brown solid and 604-1 (1.0 g, 21%) as a light-brown solid. MS (ESI) m/z 302 [M+H]⁺.

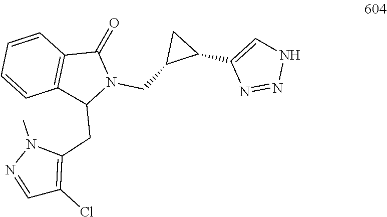 [SMILES: Cn1ncc(Cl)c1CC1c2ccccc2C(=O)N1C[C@@H]1C[C@@H]1c1c[nH]nn1]

The title compound 604 was prepared according to the procedure described for compound 548 using 604-1 as a starting material to afford a white solid (12 mg, 12%). MS (ESI) m/z 383 [M+H]⁺. ¹H NMR (400 MHz, CD₃OD) δ 7.72-7.57 (m, 2H), 7.50-7.39 (m, 3H), 6.87-6.78 (m, 1H), 5.16-5.13 (m, 0.55H), 4.76-4.73 (m, 0.45H), 3.95-3.90 (m, 1H), 3.57-3.44 (m, 3.55H), 3.34-3.25 (m, 0.45H), 3.04-3.01 (m, 0.55H), 2.89-2.62 (m, 1H), 2.32-2.12 (m, 1H), 1.72-1.54 (m, 1H), 1.38-1.15 (m, 2.5H).

### (2s,4S)-2-(((R)-1-((4-chloro-1-methyl-1H-pyrazol-5-yl)methyl)-4,6-difluoro-3-oxoisoindolin-2-yl)methyl)-5-oxa-7-azaspiro[3.4]octan-6-one (549) and (2s,4R)-2-(((S)-1-((4-chloro-1-methyl-1H-pyrazol-5-yl)methyl)-4,6-difluoro-3-oxoisoindolin-2-yl)methyl)-5-oxa-7-azaspiro[3.4]octan-6-one (550)

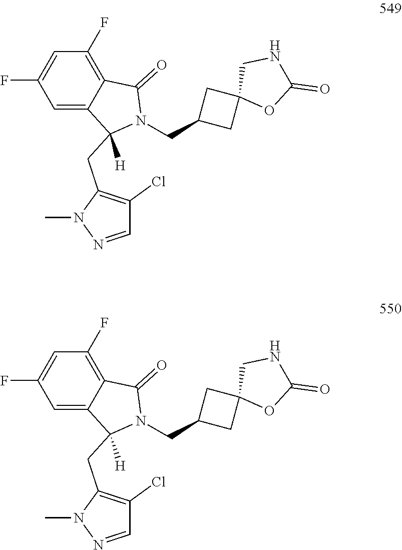 [SMILES: Cn1ncc(Cl)c1C[C@@H]1c2cc(F)cc(F)c2C(=O)N1C[C@H]1C[C@]2(CNC(=O)O2)C1 ; Cn1ncc(Cl)c1C[C@H]1c2cc(F)cc(F)c2C(=O)N1C[C@H]1C[C@]2(CNC(=O)O2)C1]

Chiral separation of compound 496 by chiral prep-HPLC (column: CHIRALPAK ID; column size: 2 cm×25 cm, 5 μm; mobile phase A: (Hex:DCM=3:1) (0.5% 2 mM NH₃-MeOH); mobile phase B: EtOH) was conducted to afford two enantiomers: compound 549 and compound 550. One of the two enantiomers was a white solid, with RT=3.877 min (CHIRAL HPLC, column: CHIRALPAK ID-3; column size: 4.6*50 mm, 3 μm; mobile phase: (Hex:DCM=3:1) (0.10% DEA):EtOH=90:10; flow: 1.0 mL/min). MS (EST) m/z 437 [M+H]⁺. ¹H NMR (400 MHz, DMSO-d₆) δ 7.56 (s, 1H), 7.50 (s, 1H), 7.36 (td, J=9.8, 2.0 Hz, 1H), 6.58 (dd, J=8.2, 2.0 Hz, 1H), 4.92 (t, J=6.8 Hz, 1H), 3.93 (dd, J=14.2, 6.1 Hz, 1H), 3.71 (s, 3H), 3.52 (dd, J=1 4.8, 6.0 Hz, 1H), 3.49 (s, 2H), 3.25 (dd, J=14.2, 6.1 Hz, 1H), 3.02 (dd, J=14.9, 7.9 Hz, 1H), 2.39-2.31 (m, 1H), 2.30-2.22 (m, 1H), 2.23-2.10 (m, 2H), 2.10-2.02 (m, 1H). The other one of the two enantiomers was a white solid, with RT=4.624 min (CHIRAL HPLC, column: CHIRALPAK ID-3; column size: 4.6*50 mm, 3 μm; mobile phase: (Hex:DCM=3:1) (0.1% DEA): EtOH=90:10; flow: 1.0 mL/min). MS (ESI) m/z 437 [M+H]⁺. ¹H NMR (400 MHz, DMSO-d₆) δ 7.56 (s, 1H), 7.50 (s, 1H), 7.36 (td, J=9.8, 2.0 Hz, 1H), 6.58 (dd, J=8.2, 2.0 Hz, 1H), 4.92 (t, J=6.8 Hz, 1H), 3.93 (dd, J=14.2, 6.1 Hz, 1H), 3.71 (s, 3H), 3.52 (dd, J=14.8, 6.0 Hz, 1H), 3.49 (s, 2H), 3.25 (dd, J=14.2, 6.1 Hz, 1H), 3.02 (dd, J=14.9, 7.9 Hz, 1H), 2.39-2.31 (m, 1H), 2.30-2.22 (m, 1H), 2.23-2.10 (m, 2H), 2.10-2.02 (m, 1H).

### (2s,4R)-2-(((S)-1-((4-chloro-1-methyl-1H-pyrazol-5-yl)methyl)-3-oxoisoindolin-2-yl)methyl)-7-methyl-5-oxa-7-azaspiro[3.4]octan-6-one (551)

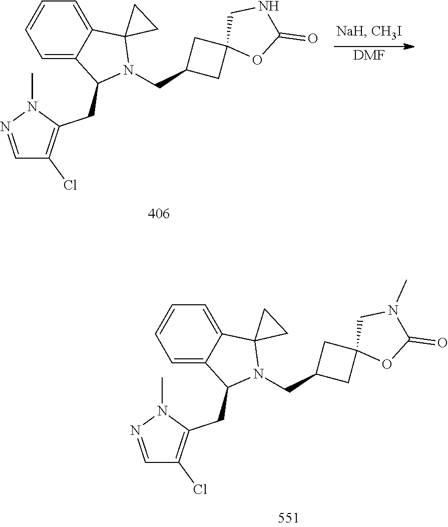

To a solution of 406 (50 mg, 0.12 mmol) in DMF (1 mL) was added NaH (3 mg, 0.13 mmol) at 0° C. under N₂. The mixture was stirred for 30 min. Then iodomethane (18 mg, 0.12 mmol) was added and the reaction was allowed to warm to r.t. This suspension was allowed to be stirred for an additional 2 h. The solvent was removed in vacuo and the residue was purified by prep-HPLC to give 551 (28 mg, 51%) as a light yellow solid. MS (ESI) m/z 415 [M+H]⁺. ¹H NMR (400 MHz, DMSO-d₆) δ 7.72-7.64 (m, 1H), 7.56 (s, 1H), 7.53-7.44 (m, 2H), 6.82-6.73 (m, 1H), 4.89 (dd, J=8.7, 5.6 Hz, 1H), 4.01 (dd, J=14.2, 5.9 Hz, 1H), 3.64 (s, 3H), 3.57 (s, 2H), 3.56 (dd, J=15.0, 5.8 Hz, 1H), 3.34 (dd, J=14.1, 6.0 Hz, 1H), 2.87 (dd, J=14.8, 8.7 Hz, 1H), 2.71 (s, 3H), 2.43-2.33 (m, 1H), 2.33-2.14 (m, 3H), 2.14-2.05 (m, 1H).

### 3-bromo-2-((2-((3-methyl-2-oxo-2,3-dihydrobenzo[d]oxazol-6-yl)methyl)-3-oxoisoindolin-1-yl)methyl)benzonitrile (552)

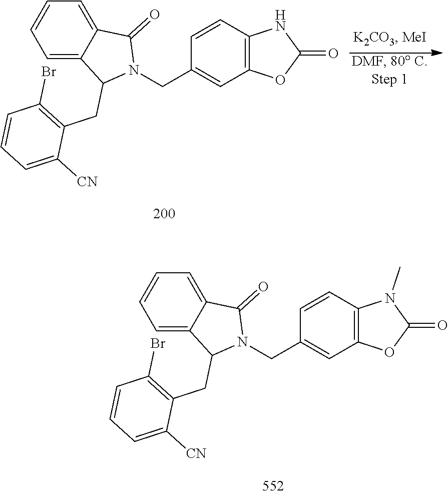 [SMILES: N#Cc1cccc(Br)c1CC1c2ccccc2C(=O)N1Cc1ccc2[nH]c(=O)oc2c1 ; "DMF, 80° C.Step 1" ; Cn1c(=O)oc2cc(CN3C(=O)c4ccccc4C3Cc3c(Br)cccc3C#N)ccc21]

The mixture of 200 (50 mg, 0. 1 mmol), K₂CO₃ (29 mg, 0.21 mmol) and Mel (29 mg, 0.21 mmol) in dried DMF (10 mL) was stirred at 80° C. for 16 h. The mixture was diluted with ethyl acetate (10 mL), washed with water (10 mL) and brine (10 mL), dried over Na₂SO₄, filtered, and concentrated in vacuo. The residue was purified by column chromatography on silica gel (eluting PE/EA=1/1 to 1/2) to give 552 (30 mg, 58%) as a white solid. MS (ESI) m/z 488 [M+H]⁺. ¹H NMR (400 MHz, CDCl₃) δ 7.94 (d, J=7.5 Hz, 11H), 7.87 (dd, J=8.2, 1.3 Hz, 11H), 7.68 (dd, J=7.7, 1.3 Hz, 1H), 7.49 (t, J=7.5 Hz, 1H), 7.43-7.31 (m, 2H), 7.10 (dd, J=7.9, 1.6 Hz, 1H), 7.02 (d, J=1.5 Hz, 11H), 6.87 (d, J=8.0 Hz, 11H), 6.57 (d, J=7.7 Hz, 11H), 5.37 (d, J=15.3 Hz, 1H), 4.88 (dd, J=8.5, 6.5 Hz, 1H), 4.34 (d, J=15.3 Hz, 1H), 3.70 (dd, J=13.6, 6.4 Hz, 1H), 3.38 (s, 3H), 3.11 (dd, J=13.6, 8.7 Hz, 1H).

### (2s,4S)-2-(((R)-6-chloro-1-((4-chloro-1-methyl-1H-pyrazol-5-yl)methyl)-4-fluoro-3-oxoisoindolin-2-yl)methyl)-5-oxa-7-azaspiro[3.4]octan-6-one (553) and (2s,4R)-2-(((S)-6-chloro-1-((4-chloro-1-methyl-1H-pyrazol-5-yl)methyl)-4-fluoro-3-oxoisoindolin-2-yl)methyl)-5-oxa-7-azaspiro[3.4]octan-6-one (554)

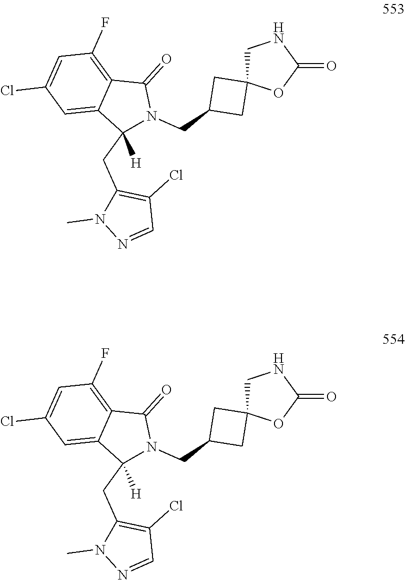 [SMILES: Cn1ncc(Cl)c1C[C@@H]1c2cc(Cl)cc(F)c2C(=O)N1C[C@H]1C[C@]2(CNC(=O)O2)C1 ; Cn1ncc(Cl)c1C[C@H]1c2cc(Cl)cc(F)c2C(=O)N1C[C@H]1C[C@]2(CNC(=O)O2)C1]

Chiral separation of compound 510 by chiral prep-HPLC (column: CHIRAL Cellulose SB; column size: 2 cm×25 cm, 5 μm; mobile phase A: MTBE; mobile phase B: EtOH) was conducted to afford two enantiomers: compound 553 and compound 554. One of the two enantiomers was a white solid, with RT=3.614 min (CHIRAL HPLC, column: CHIRAL Cellulose-SB; column size: 4.6*100 mm, 3 μm; MtBE(0.1% DEA):EtOH=70:30; flow: 1 mL/min). MS (ESI) m/z 453 [M+H]⁺. ¹H NMR (400 MHz, DMSO-d₆) δ 7.55 (s, 1H), 7.53 (dd, J=9.3, 1.6 Hz, 1H), 7.48 (s, 1H), 6.80 (d, J=1.5 Hz, 1H), 4.92 (dd, J=7.8, 5.8 Hz, 1H), 3.93 (dd, J=14.2, 6.1 Hz, 1H), 3.71 (s, 3H), 3.53 (dd, J=15.0, 5.8 Hz, 1H), 3.49 (s, 2H), 3.28 (dd, J=14.1, 6.2 Hz, 1H), 3.02 (dd, J=14.9, 7.9 Hz, 1H), 2.39-2.32 (m, 1H), 2.31-2.23 (m, 11H), 2.22-2.12 (m, 2H), 2.11-2.02 (m, 11H). The other one of the two enantiomers was a white solid, with RT=4.417 min (CHIRAL HPLC, column: CHIRAL Cellulose-SB; column size: 4.6*100 mm, 3 μm; MtBE(0.1% DEA):EtOH=70:30; flow: 1 mL/min). MS (ESI) m/z 453 [M+H]⁺. ¹H NMR (400 MHz, DMSO-d₆) δ 7.55 (s, 1H), 7.53 (dd, J=9.3, 1.6 Hz, 1H), 7.50 (s, 1H), 6.80 (d, J=1.5 Hz, 1H), 4.92 (dd, J=7.8, 5.8 Hz, 11H), 3.93 (dd, J=14.2, 6.1 Hz, 11H), 3.71 (s, 3H), 3.53 (dd, J=15.0, 5.8 Hz, 11H), 3.49 (s, 2H), 3.28 (dd, J=14.1, 6.2 Hz, 11H), 3.02 (dd, J=14.9, 7.9 Hz, 1H), 2.39-2.32 (m, 1H), 2.31-2.23 (m, 1H), 2.22-2.12 (m, 2H), 2.11-2.02 (m, 1H).

### 3-((4-chloro-1-methyl-1H-pyrazol-5-yl)methyl)-2-((4-fluoro-5-methoxypyridin-2-yl)methyl)isoindolin-1-one (555)

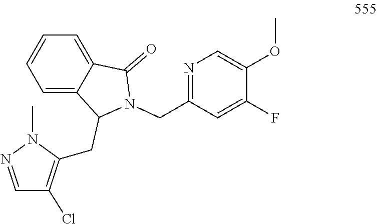 [SMILES: COc1cnc(CN2C(=O)c3ccccc3C2Cc2c(Cl)cnn2C)cc1F]

The title compound 555 was prepared according to the procedure described for compound 502 as a white solid (113 mg, 44%). MS (ESI) m/z 401 [M+H]⁺. ¹H NMR (400 MHz, DMSO-d₆) δ 7.76-7.68 (m, 1H), 7.65-7.59 (m, 1H), 7.58-7.47 (m, 3H), 7.20 (d, J=8.0 Hz, 1H), 6.95-6.87 (m, 1H), 5.12 (d, J=16.0 Hz, 1H), 4.86 (dd, J=8.0, 6.0 Hz, 1H), 4.35 (d, J=15.6 Hz, 1H), 3.85 (s, 3H), 3.63 (s, 3H), 3.51 (dd, J=14.8, 6.0 Hz, 1H), 3.00 (dd, J=14.8, 8.4 Hz, 1H).

The intermediate 555-5 was prepared as follows:

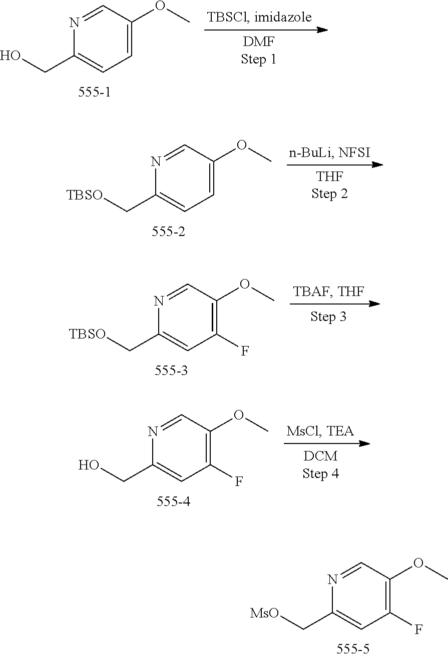

Step 1 was performed according to the procedure outlined for the preparation of 360-3.

### Step 2. 2-(((tert-butyldimethylsilyl)oxy)methyl)-4-fluoro-5-methoxypyridine (555-3)

To a mixture of 2-(((tert-butyldimethylsilyl)oxy)methyl)-5-methoxypyridine (200 mg, 0.79 mmol) in THF (10 mL) was added n-BuLi (0.5 mL, 2.5 mol/L in THF) at −70° C. The mixture was stirred at −70° C. for 1 h. NFSI (400 mg, 1.26 mmol) was added and the mixture was stirred at −70° C. for another 1 h. Water (20 mL) was added and extracted with EA (20 mL*3). The combined organic layers were washed with H₂O (100 mL), dried over anhydrous Na₂SO₄, filtered and concentrated. The residue was purified by FCC (PE/EA=10/1) to give 555-3 (70 mg, 32%) as a yellow oil. MS (ESI) m/z 272 [M+H]⁺.

Step 3 was performed according to the procedure outlined for the preparation of 360-5.

Step 4 was performed according to the procedure outlined for the preparation of 132-2.

### 3-((4-chloro-1-methyl-1H-pyrazol-5-yl)methyl)-2-((3-oxocyclobutyl)methyl)isoindolin-1-one (556)

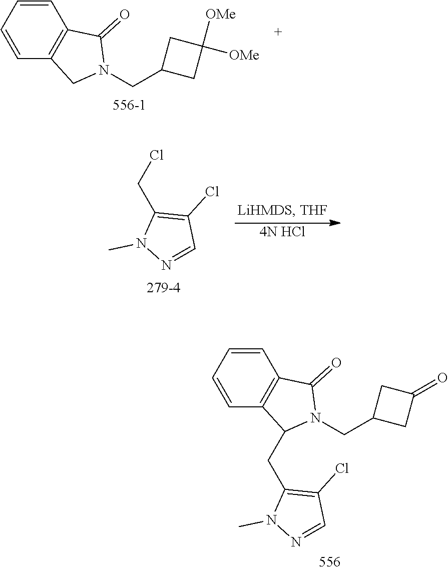

To a solution of 2-[(3,3-dimethoxycyclobutyl)methyl]isoindolin-1-one (4.45 g, 17.0 mmol) in THF (100 mL) was added LiHMDS (19.3 mmol) at −78° C. under N₂. The mixture was stirred for 30 min. Then 279-4 (2.90 g, 17.6 mmol) in THF (20 mL) was added. This suspension was allowed to be stirred for an additional I h. Then the mixture was warmed up to r.t. 4N HCl (30 mL) was added to the mixture. The mixture was stirred overnight, quenched with sat. NaHCO₃ (30 mL) and then extracted with EA. The organic layer was separated, dried over anhydrous Na₂SO₄ and concentrated under vacuum. The residue was purified by column chromatography to give 556 (2.05 g, 33.93%) as a white solid. MS (ESI) m/z 344 [M+H]⁺. ¹H NMR (400 MHz, CDCl₃) δ 7.88 (d, J=7.5 Hz, 1H), 7.55-7.40 (m, 3H), 6.56 (d, J=7.4 Hz, 1H), 4.93 (dd, J=9.8, 5.6 Hz, 1H), 4.34 (dd, J=14.3, 8.8 Hz, 1H), 3.53 (dd, J=14.4, 5.6 Hz, 1H), 3.47 (s, 3H), 3.41 (dd, J=14.3, 6.5 Hz, 11H), 3.29-3.20 (m, 1H), 3.16-3.08 (m, 1H), 3.06-2.77 (m, 3H), 2.63 (dd, J=14.4, 9.8 Hz, 1H).

### 3-((4-chloro-1-methyl-1H-pyrazol-5-yl)methyl)-2-((1-methyl-1H-benzo[d][1,2,3]triazol-5-yl)methyl)isoindolin-1-one (557)

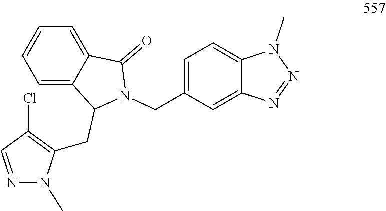 [SMILES: Cn1ncc(Cl)c1CC1c2ccccc2C(=O)N1Cc1ccc2c(c1)nnn2C]

The title compound 557 was prepared according to the procedure described for compound 502 as a white solid (30 mg, 16%). MS (ESI) m/z 407 [M+H]⁺. ¹H NMR (400 MHz, CDCl₃) δ 7.97-7.91 (m, 2H), 7.53-7.40 (m, 5H), 6.60 (d, J=7.5 Hz, 1H), 5.54 (d, J=15.2 Hz, 1H), 4.67 (dd, J=9.2, 6.0 Hz, 1H), 4.39 (d, J=15.2 Hz, 1H), 4.29 (s, 3H), 3.45 (s, 3H), 3.43 (dd, J=14.5, 6.0 Hz, 1H), 2.69 (dd, J=14.5, 9.2 Hz, 11H).

The intermediate 557-4 was prepared as follows:

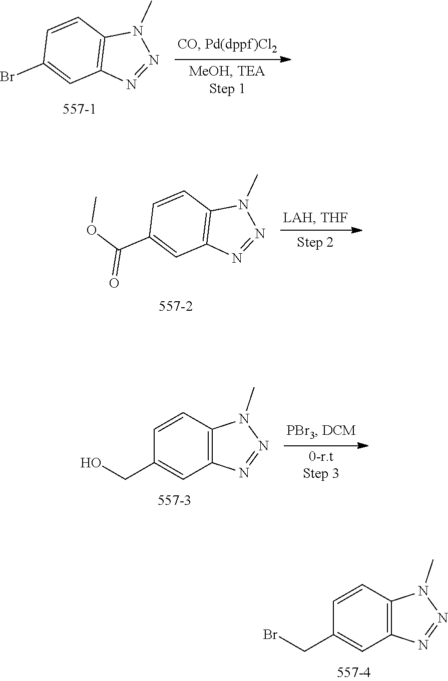

### Step 1: methyl 1-methyl-1H-benzo[d][1,2,3]triazole-5-carboxylate (557-2)

The mixture of 5-bromo-1-methyl-1H-benzo[d][1,2,3]triazole (700 mg, 3.3 mmol), TEA (3.3 g, 33 mmol) and Pd(dppf)Cl₂ (239 mg, 0.33 mmol) in dried MeOH (15 mL) was stirred at 80° C. for 16 h under CO atmosphere. The mixture was diluted with ethyl acetate (20 mL), the organic layer was washed with water (20 mL) and brine (20 mL), dried over Na₂SO₄, filtered, and concentrated in vacuo. The residue was purified by column chromatography on silica gel (eluting PE/EA=2/1 to 1/1) to give 557-2 (400 mg, 63%) as a yellow oil. MS (ESI) m/z 192 [M+H]⁺.

Step 2 was performed according to the procedure outlined for the preparation of 217-2.

Step 3 was performed according to the procedure outlined for the preparation of 217-3.

### 3-((4-chloro-1-methyl-1H-pyrazol-5-yl)methyl)-2-((1-methyl-1H-benzo[d][1,2,3]triazol-6-yl)methyl)isoindolin-1-one (558) and 3-((4-chloro-1-methyl-1H-pyrazol-5-yl)methyl)-2-((2-methyl-2H-benzo[d][1,2,3]triazol-5-yl)methyl)isoindolin-1-one (559)

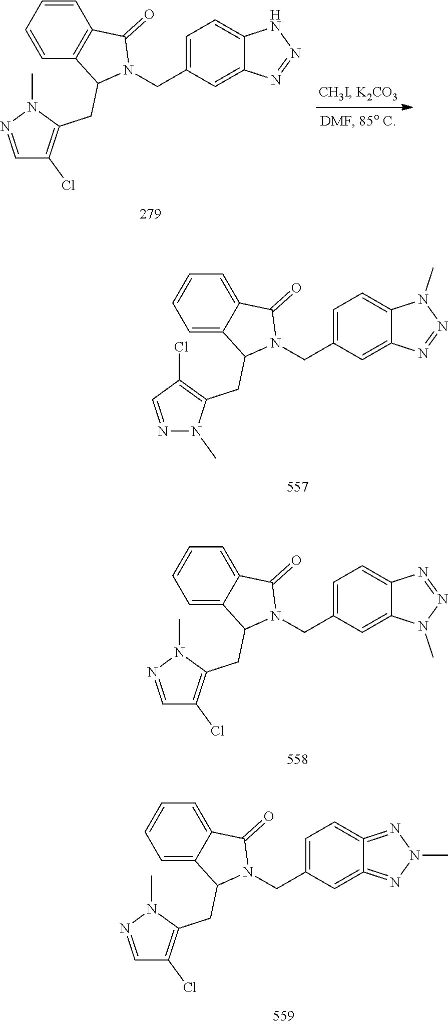

To a solution of 279 (95 mg, 0.24 mmol) in DMF (5 mL) was added CH₃I (68 mg, 0.48 mmol) and K₂CO₃ (66 mg, 0.48 mmol). The mixture was stirred for 16 h at 85° C., then quenched with water (5 mL) and extracted with ethyl acetate (10 mL*2), The combined organic layer was washed with water (10 mL) and brine (10 mL), dried over Na₂SO₄, filtered, and concentrated in vacuo. The residue was purified by pre-HPLC to give 3 isomers: 557, 558 and 559.

The first one of the 3 isomers was compound 557, with RT=2.39 min (Column: Agilent poroshell 120 SB C18+, 2.7 um 4.6*50 mm; Mobile phase: ACN(0.05% FA)-Water(0.05% FA); Gradient: 5% ACN to 95% ACN in 1.0 min, hold 1.0 min, total 5 min; Flow rate:1.8 mL/min; Column Temp: 40 degree).

The second one of the 3 isomers was a white solid, with RT=2.41 min (Column: Agilent poroshell 120 SB C18+, 2.7 um 4.6*50 mm; Mobile phase: CAN (0.05% FA)-Water(0.05% FA); Gradient: 5% ACN to 95% ACN in 1.0 min, hold 1.0 min, total 5 min; Flow rate:1.8 mL/min; Column Temp: 40 degree). MS (ESI) m/z 407 [M+H]⁺. ¹H NMR (400 MHz, CDCl₃) δ 7.94 (d, J=7.5 Hz, 11H), 7.80 (dd, J=8.8, 0.9 Hz, 1H), 7.70 (s, 11H), 7.52-7.41 (m, 3H), 7.30 (dd, J=8.8, 1.5 Hz, 1H), 6.60 (d, J=7.2 Hz, 1H), 5.51 (d, J=15.2 Hz, 1H), 4.68 (dd, J=9.2, 6.0 Hz, 1H), 4.50 (s, 3H), 4.36 (d, J=15.2 Hz, 1H), 3.43 (s, 3H), 3.42 (dd, J=14.5, 6.0 Hz, 1H), 2.68 (dd, J=14.5, 9.2 Hz, 1H).

The third one of the 3 isomers was a white solid, with RT=2.50 min (Column: Agilent poroshell 120 SB C18+, 2.7 um 4.6*50 mm; Mobile phase: ACN(0.05% FA)-Water(0.05% FA); Gradient: 5% ACN to 95% ACN in 1.0 min, hold 1.0 min, total 5 min; Flow rate:1.8 mL/min; Column Temp:40 degree). MS (ESI) m/z 407 [M+H]⁺. ¹H NMR (400 MHz, CDCl₃) δ 7.99 (d, J=8.4 Hz, 1H), 7.95 (d, J=7.6 Hz, 1H), 7.55-7.42 (m, 3H), 7.31-7.26 (m, 2H), 6.73 (d, J=7.4 Hz, 1H), 5.55 (d, J=15.1 Hz, 1H), 4.67 (t, J=7.5 Hz, 1H), 4.32 (d, J=15.1 Hz, 1H), 4.28 (s, 3H), 3.44 (s, 3H), 3.36 (dd, J=14.7, 6.7 Hz, 1H), 2.78 (dd, J=14.6, 8.3 Hz, 1H).

### 3-((4-chloro-1-methyl-1H-pyrazol-5-yl)methyl)-2-(3,5-difluoro-4-methoxybenzyl)isoindolin-1-one (560)

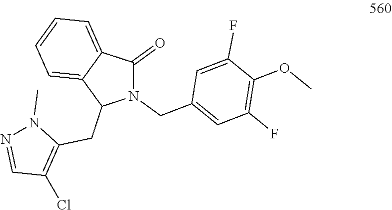 [SMILES: COc1c(F)cc(CN2C(=O)c3ccccc3C2Cc2c(Cl)cnn2C)cc1F]

The title compound 560 was prepared according to the procedure described for 502 as a white solid (9 mg, 19%). MS (ESI) m/z 418 [M+H]⁺. ¹H NMR (400 MHz, CDCl₃) δ 7.92 (d, J=7.3 Hz, 1H), 7.55-7.42 (m, 3H), 6.86-6.75 (m, 2H), 6.67 (d, J=7.4 Hz, 1H), 5.25 (d, J=15.4 Hz, 1H), 4.71 (dd, J=8.9, 6.2 Hz, 1H), 4.11 (d, J=15.4 Hz, 1H), 3.97 (s, 3H), 3.49 (s, 3H), 3.35 (dd, J=14.5, 6.2 Hz, 1H), 2.71 (dd, J=14.5, 9.0 Hz, 1H).

### 2-(benzo[d]oxazol-5-ylmethyl)-3-((4-chloro-1-methyl-1H-pyrazol-5-yl)methyl)isoindolin-1-one (561)

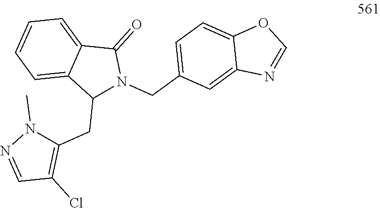 [SMILES: Cn1ncc(Cl)c1CC1c2ccccc2C(=O)N1Cc1ccc2ocnc2c1]

The title compound 561 was prepared according to the procedure described for 502 as a white solid (30 mg, 20%). MS (ESI) m/z 393 [M+H]⁺. ¹H NMR (400 MHz, CDCl₃) δ 8.10 (s, 1H), 7.93 (d, J=7.5 Hz, 1H), 7.69 (d, J=1.7 Hz, 1H), 7.56-7.49 (m, 2H), 7.48 (s, 1H), 7.45-7.40 (m, 1H), 7.32 (dd, J=8.4, 1.6 Hz, 1H), 6.61 (d, J=7.5 Hz, 1H), 5.51 (d, J=15.2 Hz, 1H), 4.68 (dd, J=9.2, 6.0 Hz, 1H), 4.35 (d, J=15.2 Hz, 1H), 3.44 (s, 3H), 3.41 (dd, J=14.5, 6.1 Hz, 1H), 2.69 (dd, J=14.5, 9.2 Hz, 1H).

### 3-((1-((4-chloro-1-methyl-1H-pyrazol-5-yl)methyl)-3-oxoisoindolin-2-yl)methyl)cyclobutane-1-carboxamide (562)

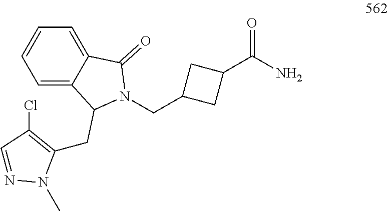 [SMILES: Cn1ncc(Cl)c1CC1c2ccccc2C(=O)N1CC1CC(C(N)=O)C1]

The title compound 562 was prepared according to the procedure described for compound 404 as an off-white solid (12 mg, 22.6%). MS (ESI) m/z 373 [M+H]⁺. ¹H NMR (400 MHz, DMSO-d₆) δ 7.71-7.63 (m, 1H), 7.55 (s, 1H), 7.53-7.44 (m, 2H), 7.13 (d, J=10.7 Hz, 1H), 6.81-6.72 (m, 1H), 6.68 (s, 1H), 4.92-4.81 (m, 1H), 4.05 (dd, J=14.0, 9.6 Hz, 0.5H), 3.91 (dd, J=14.2, 7.8 Hz, 0.5H), 3.64 (d, J=4.9 Hz, 3H), 3.54-3.53 (m, 1H), 3.35-3.18 (m, 1H), 3.06-2.73 (m, 2H), 2.47-2.43 (m, 1H), 2.31-2.11 (m, 1H), 2.10-1.87 (m, 2H), 1.86-1.71 (m, 1H).

### 2-((3-((4-chloro-1-methyl-1H-pyrazol-5-yl)methyl)-5-fluoro-1,1-dioxidobenzo[d]isothiazol-2(3H)-yl)methyl)-5-oxa-7-azaspiro[3.4]octan-6-one (563)

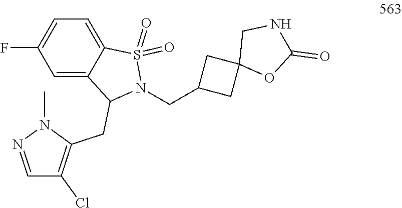 [SMILES: Cn1ncc(Cl)c1CC1c2cc(F)ccc2S(=O)(=O)N1CC1CC2(CNC(=O)O2)C1]

The title compound 563 was prepared according to the procedure described for compound 270 as a white solid (26 mg, 24.5%). MS (ESI) m/z 455 [M+H]⁺. ¹H NMR (400 MHz, DMSO-d₆) δ 7.99 (dd, J=8.6, 4.9 Hz, 1H), 7.55 (s, 1H), 7.54-7.41 (m, 2H), 7.06 (dd, J=9.0, 2.3 Hz, 1H), 4.87 (t, J=7.1 Hz, 1H), 3.80 (s, 3H), 3.49-3.39 (m, 2H), 3.38-3.25 (m, 2H), 3.21-3.11 (m, 2H), 2.32-2.18 (m, 2H), 2.10-1.93 (m, 3H).

The intermediate 563-6 was prepared as follows:

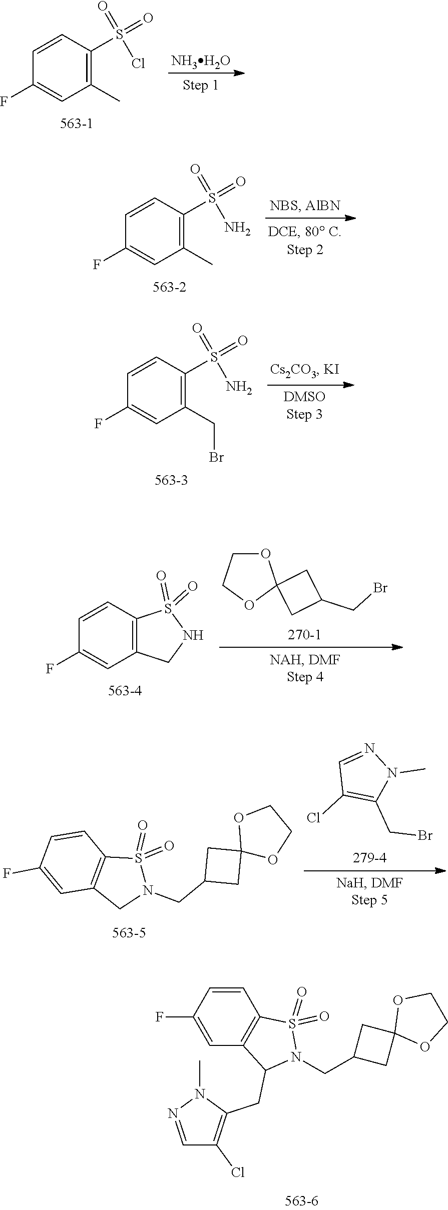

### Step 1. 4-fluoro-2-methylbenzenesulfonamide (563-2)

To a solution of ammonium hydroxide (10 mL) was added 563-1 (5 g, 24.0 mmol) in THF (50 mL) at 0′C under N₂. The mixture was stirred for 1 h, then diluted with EA, and washed with water. The organic layer was separated, dried over anhydrous Na₂SO₄ and concentrated under vacuum to give 563-2 (3.8 g) as a white solid, which was used in the next step without further purification. MS (ESI) m/z 190 [M+H]⁺.

Step 2 was performed according to the procedure outlined for the preparation of 260-3.

### Step 3. 5-fluoro-2,3-dihydrobenzo[d]isothiazole 1,1-dioxide (563-4)

To a solution of 563-3 (1 g, 3.73 mmol) in DMSO (10 mL) were added Cs₂CO₃ (1.82 g, 5.59 mmol) and KI (62 mg, 0.37 mmol) under N₂. The mixture was stirred at 80′C for 2 h. The mixture was diluted with EA and washed with water. The organic layer was separated, dried over anhydrous Na₂SO₄ and concentrated under vacuum. The residue was purified by prep-TLC (PE/EA=1.5/1) to give 563-4 (230 mg, 32%) as a white solid. MS (ESI) m/z 188 [M+H]⁺.

Step 4 was performed according to the procedure outlined for the preparation of 222-2.

### Step 5. 2-((5,8-dioxaspiro[3.4]octan-2-yl)methyl)-3-((4-chloro-1-methyl-1H-pyrazol-5-yl)methyl)-5-fluoro-2,3-dihydrobenzo[d]isothiazole 1,1-dioxide (563-6)

To a solution of 563-5 (200 mg, 0.638 mmol) in DMF (1 mL) was added NaH (17 mg, 0.70 mmol) at 0° C.C under N₂. The mixture was stirred for 30 min. Then 279-4 (134 mg, 0.63 mmol) was added and the reaction was allowed to warm to r.t. This suspension was stirred at 80′C for additional 2 h. Water was added at 0° C.C and then extracted with EA. The organic layer was separated, dried over anhydrous Na₂SO₄ and concentrated under vacuum. The residue was purified by prep-TLC (PE/EA=2/1) to give 563-6 (100 mg, 35%) as a yellow solid. MS (ESI) m/z 442 [M+H]⁺.

### 2-((2-oxaspiro[3.3]heptan-6-yl)methyl)-3-((4-chloro-1-methyl-1H-pyrazol-5-yl)methyl)isoindolin-1-one (564)

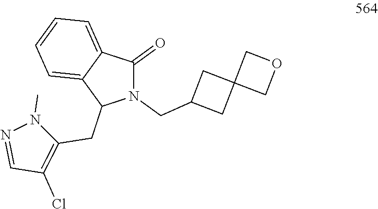 [SMILES: Cn1ncc(Cl)c1CC1c2ccccc2C(=O)N1CC1CC2(COC2)C1]

The title compound 564 was prepared according to the procedure described for compound 502 as an off-white solid (14 mg, 15.6%). MS (ESI) m/z 372 [M+H]⁺. ¹H NMR (400 MHz, DMSO-d₆) δ 7.72-7.62 (m, 1H), 7.55 (s, 1H), 7.53-7.44 (m, 2H), 6.83-6.75 (m, 1H), 4.85 (dd, J=8.6, 5.8 Hz, 1H), 4.58-4.44 (m, 4H), 3.87 (dd, J=14.0, 8.2 Hz, 1H), 3.63 (s, 3H), 3.49 (dd, J=14.8, 5.8 Hz, 1H), 3.20 (dd, J=14.0, 6.4 Hz, 1H), 2.85 (dd, J=14.8, 8.6 Hz, 1H), 2.44-2.28 (m, 2H), 2.25-2.14 (m, 1H), 2.04-1.95 (m, 1H), 1.88-1.79 (m, 1H).

The intermediate 564-3 was prepared as follows:

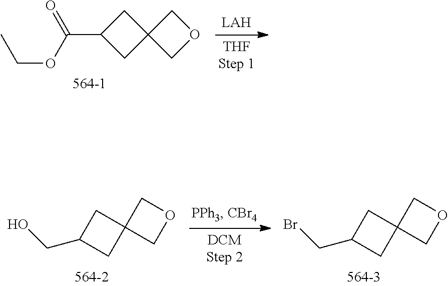

Step 1 was performed according to the procedure outlined for the preparation of 217-2.

Step 2 was performed according to the procedure outlined for the preparation of 217-3.

### 2-((3-((4-chloro-1-methyl-1H-pyrazol-5-yl)methyl)-1,1-dimethylisoindolin-2-yl)methyl)-5-oxa-7-azaspiro[3.4]octan-6-one (565)

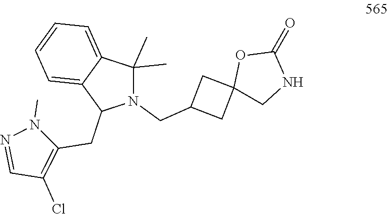 [SMILES: Cn1ncc(Cl)c1CC1c2ccccc2C(C)(C)N1CC1CC2(CNC(=O)O2)C1]

The title compound 565 was prepared according to the procedure described for compound 270 as a white solid (25 mg, 6.5%). MS (ESI) m/z 415 [M+H]⁺. ¹H NMR (400 MHz, DMSO-d₆) δ 7.52 (s, 1H), 7.48 (s, 1H), 7.29-7.19 (m, 2H), 7.13-7.01 (m, 1H), 6.29 (d, J=7.5 Hz, 1H), 4.42 (dd, J=9.1, 5.2 Hz, 1H), 3.56-3.49 (m, 4H), 3.35 (s, 2H), 3.26 (dd, J=14.4, 5.3 Hz, 1H), 3.05 (dd, J=13.4, 5.9 Hz, 1H), 2.80-2.65 (m, 1H), 2.42-2.26 (m, 2H), 2.25-2.13 (m, 1H), 2.07-1.94 (m, 2H), 1.38 (s, 3H), 1.26 (s, 3H).

The intermediate 565-10 was prepared as follows:

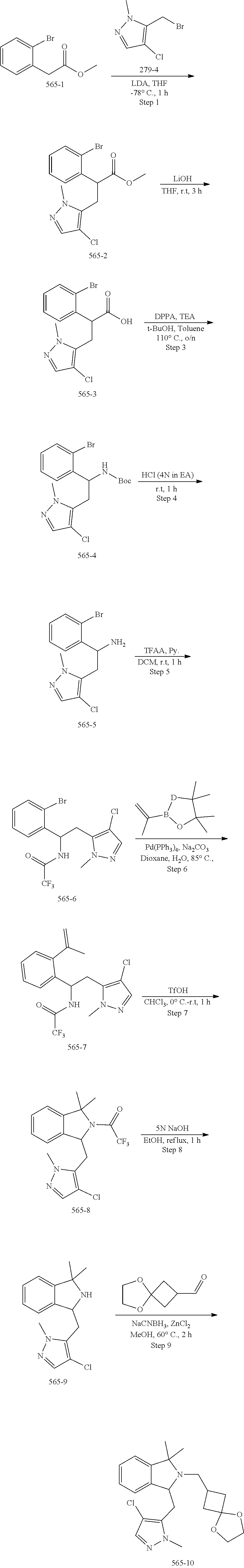

### Step 1. methyl 2-(2-bromophenyl)-3-(4-chloro-1-methyl-IH-pyrazol-5-yl)propanoate (565-2)

To a solution of methyl 2-(2-bromophenyl)acetate (1.50 g, 6.55 mmol) in THF (20 mL) was added LDA (1M in THF) (8.5 mL, 8.51 mmol) dropwise at −78° C. under N₂. The mixture was stirred at −78° C. for 30 min. Then 279-4 (1.37 g, 6.55 mmol) was added. The resulting mixture was stirred at −78° C. for 1 h. The reaction mixture was quenched with sat. NH₄Cl (aq.) and extracted with ethyl acetate. The combined organic phase was dried over Na₂SO₄ and concentrated. The residue was purified by silica gel column eluting with PE/EA (5/1) to afford 565-2 (1.38 g, 58.9%) as a light yellow oil. MS (ESI) m/z 357/359 [M+H]⁺.

### Step 2. 2-(2-bromophenyl)-3-(4-chloro-1-methyl-1H-pyrazol-5-yl)propanoic acid (565-3)

LiOH (1 M in water) (12 mL) was added to a stirred solution of 565-2 (1.38 g, 3.86 mmol) in THF (20 mL). The mixture was stirred at r.t. for 3 h. The mixture was diluted with water and extracted with ethyl acetate. The pH of aqueous phase was adjusted to about 3-4 using 1N HCl and the resulting aqueous phase was extracted with ethyl acetate. The organic phase was dried and concentrated to afford 565-3 (1.18 g, 89.0%) as a white solid. MS (ESI) m/z 343/345 [M+H]⁺.

### Step 3. tert-butyl (1-(2-bromophenyl)-2-(4-chloro-1-methyl-1H-pyrazol-5-yl)ethyl)carbamate (565-4)

To a solution of 565-3 (1.10 g, 3.20 mmol) in toluene (10 mL) was added TEA (0.89 mL, 6.40 mmol) and [azido(phenoxy)phosphoryl]oxybenzene (1.32 g, 4.80 mmol). The mixture was stirred at 110° C. for 2 h and then cooled to 50° C. Tert-butanol (1.5 mL) was added to the above mixture. The resulting mixture was stirred at 90° C. overnight. The reaction mixture was cooled to r.t. and concentrated under vacuum. The residue was purified by pre-TLC (PE/EA=2/1) to afford 565-4 (680 mg, 51.2%) as a white solid. MS (ESI) m/z 414/416 [M+H]⁺.

### Step 4. 1-(2-bromophenyl)-2-(4-chloro-1-methyl-1H-pyrazol-5-yl)ethan-1-amine (565-5)

A mixture of 565-4 (350 mg, 0.84 mmol) in HCl (4N in EA) (5.0 mL) was stirred at r.t. for 1 h. The precipitated solid was collected by filtration and washed with ethyl acetate. The collected solid was dried in an oven to afford 565-5 (245 mg, 92.3%) as a white solid. MS (ESI) m/z 314/316 [M+H]⁺.

### Step 5. N-(1-(2-bromophenyl)-2-(4-chloro-1-methyl-1H-pyrazol-5-yl)ethyl)-2,2,2-trifluoroacetamide (565-6)

To a solution of 565-5 (245 mg, 0.70 mmol) and pyridine (110 mg, 1.40 mmol) in DCM (5 mL) was added (2,2,2-trifluoroacetyl) 2,2,2-trifluoroacetate (147 mg, 0.70 mmol). The reaction mixture was stirred at r.t. for 1 h. The mixture was diluted with DCM and washed with water. The organic layer was separated, dried over anhydrous Na₂SO₄ and concentrated. The residue was purified by silica gel column eluting with PE/EA (1/3) to give 565-6 (272 mg, 94.9%) as a colorless oil. MS (ESI) m/z 410/412 [M+H]⁺.

Step 6 was performed according to the procedure outlined for the preparation of 26-1.

### Step 7. 1-(3-((4-chloro-1-methyl-1H-pyrazol-5-yl)methyl)-1,1-dimethylisoindolin-2-yl)-2,2,2-trifluoroethan-1-one (565-8)

To a solution of 565-7 (153 mg, 0.41 mmol) in chloroform (5 mL) was added trifluoromethanesulfonic acid (62 mg, 0.41 mmol) at 0° C. The mixture was stirred at r.t. for 1 h. The reaction mixture was concentrated under vacuum. The residue was quenched with sat. NaHCO₃ (aq.) and extracted with DCM. The organic layer was separated, dried over anhydrous Na₂SO₄ and concentrated. The residue was purified by pre-TLC (PE/EA=5/1) to give 565-8 (135 mg, 88.2%) as a colorless oil. MS (ESI) m/z 372 [M+H]⁺.

### Step 8. 3-((4-chloro-1-methyl-1H-pyrazol-5-yl)methyl)-1,1-dimethylisoindoline (565-9)

To a solution of 565-8 (125 mg, 0.34 mmol) in ethanol (10 mL) was added NaOH (5M in H₂O) (2.0 mL). The mixture was stirred at 100° C. for 1 h. The solvent was removed in vacuo. The residue was triturated with DCM/methanol (10/1), and then anhydrous MgSO₄ was added. The mixture was filtered and the filtrate was concentrated to give 565-9 (116 mg, crude) as a yellow solid, which was used for next step directly. MS (ESI) m/z 276 [M+H]⁺.

### Step 9. 2-((5,8-dioxaspiro[3.4]octan-2-yl)methyl)-3-((4-chloro-1-methyl-1H-pyrazol-5-yl)methyl)-1,1-dimethylisoindoline (565-10)

To a solution of 565-9 (90 mg, 0.33 mmol) and 5,8-dioxaspiro[3.4]octane-2-carbaldehyde (93 mg, 0.65 mmol) in methanol (3 mL) were added AcOH (20 mg, 0.33 mmol) and NaCNBH₃ (41 mg, 0.65 mmol) under N₂. The mixture was stirred at r.t. for 2 h. The solvent was removed in vacuo and the residue was purified by pre-TLC (PE/EA=3/1) to give 565-10 (130 mg, 99.1%) as a yellow oil. MS (ESI) m/z 402 [M+H]⁺.

### 3-((3,4-dichloro-1-methyl-1H-pyrazol-5-yl)methyl)-2-(4-methoxybenzyl)isoindolin-1-one (566)

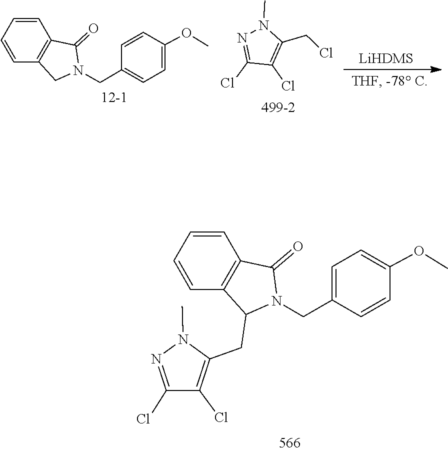

To a solution of 12-1 (400 mg, 1.5 mmol) in THF (8 mL) was added LiHMDS (2 ml, 2 mmol) dropwise at −78° C. under N₂. The mixture was stirred for 40 min at −78° C. Then 499-2 (398 mg, 2 mmol) was added into the mixture and stirred for 40 min at −78° C. The mixture was quenched with sat. NH₄Cl (aq.), diluted with water (10 mL), and extracted with EA (3*10 mL). The 15 combined organic layers were washed with brine (10 mL), dried over anhydrous Na₂SO₄, and after filtration, the filtrate was concentrated under reduced pressure. The residue was purified by prep-HPLC to give 566 (160 mg, 32%) as a white solid. MS (ESI) m/z 416 [M+H]⁺. ¹H NMR (400 MHz, CDCl₃) δ 7.91 (d, J=7.6 Hz, 1H), 7.57-7.40 (m, 2H), 7.21-7.13 (m, 2H), 6.90-6.80 (m, 2H), 6.68 (d, J=7.5 Hz, 1H), 5.36 (d, J=15.1 Hz, 1H), 4.64 (dd, J=9.2, 5.9 Hz, 1H), 4.15 (d, J=15.1 Hz, 1H), 3.79 (s, 3H), 3.41 (s, 3H), 3.36 (dd, J=14.6, 5.9 Hz, 1H), 2.64 (dd, J=14.6, 9.2 Hz, 1H).

### 3-((2-(4-methoxybenzyl)-3-oxoisoindolin-1-yl)methyl)-4-methylpicolinonitrile (567)

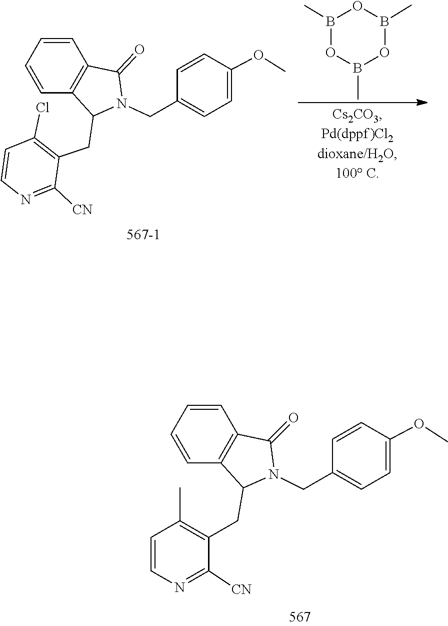

The intermediate 567-1 was prepared according to the procedure described for compound 566. The mixture of 567-1 (450 mg, 1.11 mmol), Pd(dppf)Cl₂ (17 mg, 0.02 mmol), Cs₂CO₃ (384 mg, 2.78 mmol) and trimethylboroxine (1.0 g, 5.6 mmol) in 1,4-dioxane/H₂O(10/1, 11 mL) was stirred at 100° C. for 16 h. The mixture was diluted with ethyl acetate (20 mL), washed with water (20 mL) and brine (20 mL), dried over Na₂SO₄, filtered, and concentrated in vacuo. The residue was purified by column chromatography on silica gel (eluting with PE/EA=1/1 to 1/2) to give 567 (170 mg, 40%) as a white solid. MS (ESI) m/z 384 [M+H]⁺. 11H NMR (400 MHz, CDCl₃) δ 8.54 (d, J=4.8 Hz, 1H), 7.93 (d, J=7.5 Hz, 1H), 7.48 (t, J=7.5 Hz, 1H), 7.39-7.29 (m, 2H), 7.19 (d, J=8.5 Hz, 2H), 6.84 (d, J=8.6 Hz, 2H), 6.45 (d, J=7.6 Hz, 11H), 5.28 (d, J=15.1 Hz, 1H), 4.76 (dd, J=9.0, 6.4 Hz, 1H), 4.26 (d, J=15.1 Hz, 11H), 3.78 (s, 3H), 3.60 (dd, J=13.8, 6.3 Hz, 1H), 2.86 (dd, J=13.8, 9.3 Hz, 1H), 2.01 (s, 3H).

### 2-((1-((4-chloro-1-methyl-1H-pyrazol-5-yl)methyl)-3-oxoisoindolin-2-yl)methyl)-5-thia-7-azaspiro[3.4]octan-6-one (568)

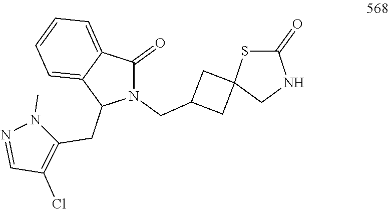 [SMILES: Cn1ncc(Cl)c1CC1c2ccccc2C(=O)N1CC1CC2(CNC(=O)S2)C1]

The title compound 568 was prepared according to the procedure described for compound 307 as a yellow solid (14 mg, 12.4%). MS (ESI) m/z 417 [M+H]⁺. ¹H NMR (400 MHz, DMSO-d₆) δ 7.85-7.83 (m, 1H), 7.55-7.50 (m, 4H), 6.80-6.73 (m, 1H), 5.20 (dd, J=8.6, 5.3 Hz, 1H), 4.75 (d, J=13.2 Hz, 1H), 3.79-3.73 (m, 2H), 3.64 (s, 3H), 3.51 (s, 2H), 2.96 (dd, J=14.8, 8.8 Hz, 1H), 2.45-2.25 (m, 5H).

The intermediate 568-1 was prepared as follows:

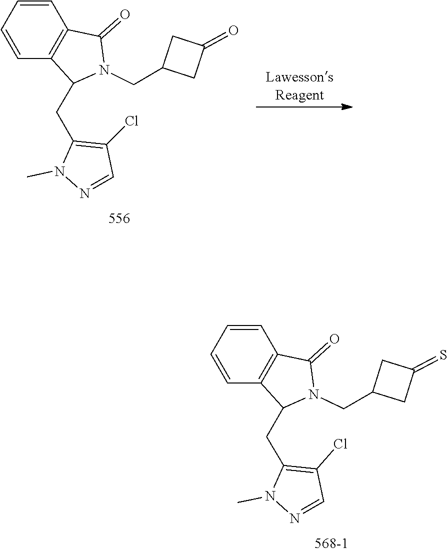 [SMILES: Cn1ncc(Cl)c1CC1c2ccccc2C(=O)N1CC1CC(=O)C1 ; Cn1ncc(Cl)c1CC1c2ccccc2C(=O)N1CC1CC(=S)C1]

A mixture of 556 (2.0 g, 5.8 mmol) and Lawesson's Reagent (1.4 g, 3.5 mmol) in toluene (50 mL) was stirred at 90° C. overnight. The mixture was concentrated and purified by FCC (PE/EA=3/1) to give 568-1 (1.0 g, 50%) as a yellow solid. MS (ESI) m/z 360 [M+H]⁺.

### (2s,4R)-2-(((S)-1-((4-chloro-1-methyl-1H-pyrazol-5-yl)methyl)-3-oxoisoindolin-2-yl)methyl)-5-oxa-7-azaspiro[3.4]octane-6,8-dione (569), 2-((1-((4-chloro-1-methyl-1H-pyrazol-5-yl)methyl)-3-oxoisoindolin-2-yl)methyl)-5-oxa-7-azaspiro[3.4]octane-6,8-dione (587) and (2s,4S)-2-(((R)-1-((4-chloro-1-methyl-1H-pyrazol-5-yl)methyl)-3-oxoisoindolin-2-yl)methyl)-5-oxa-7-azaspiro[3.4]octane-6,8-dione (644)

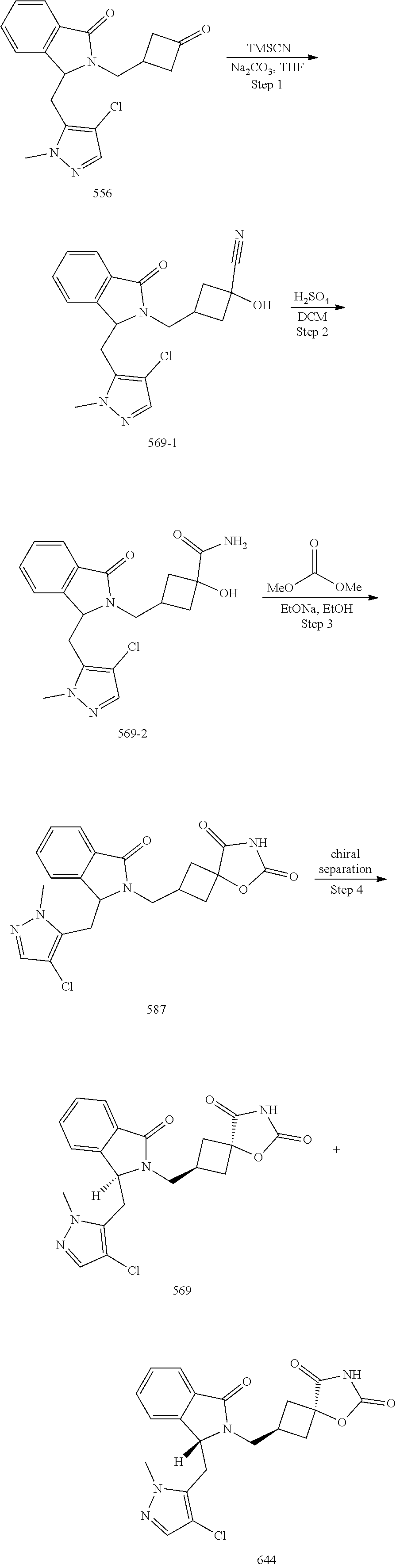

### Step 1. 3-((1-((4-chloro-1-methyl-1H-pyrazol-5-yl)methyl)-3-oxoisoindolin-2-yl)methyl)-1-hydroxycyclobutane-1-carbonitrile (569-1)

To a solution of 556 (1.0 g, 2.9 mmol) in THF (30 mL) were added Na₂CO₃ (922 mg, 8.7 mmol) and TMSCN (350 mg, 3.5 mmol) at r.t. The reaction was stirred at r.t. overnight. The mixture was added to ice-water (20 mL), and extracted with EA (20 mL*3). The combined organic layers were washed with brine (30 mL), dried over Na₂SO₄, filtered and concentrated to give crude 569-1 (1.0 g, 93%) as a white solid. MS (EST) m/z 371 [M+H]⁺.

### Step 2. 3-((1-((4-chloro-1-methyl-1H-pyrazol-5-yl)methyl)-3-oxoisoindolin-2-yl)methyl)-1-hydroxycyclobutane-1-carboxamide (569-2)

To a solution of 569-1 (600 mg, 1.6 mmol) in DCM (5 mL) was added H₂SO₄ (5 mL, 98%) at r.t. The reaction was stirred for 3 h at r.t. The mixture was poured into sat. Na₂CO₃(20 mL), extracted with DCM (15 mL*2). The combined organic layers were washed with brine (20 mL), dried over Na₂SO₄, filtered and concentrated. The residue was purified by FCC (PE/EA˜DCM/MeOH=1/1˜15/1) to give 569-2 (400 mg, 64%) as a white solid. MS (ESI) m/z 389 [M+H]⁺.

### Step 3. 2-((1-((4-chloro-1-methyl-1H-pyrazol-5-yl)methyl)-3-oxoisoindolin-2-yl)methyl)-5-oxa-7-azaspiro[3.4]octane-6,8-dione (587)

A mixture of 569-2 (100 mg, 0.76 mmol), dimethyl carbonate (116 mg, 1.3 mmol) and NaOEt (170 mg, 0.52 mmol, 21 wt % in EtOH) in EtOH (10 mL) was stirred at 85° C. overnight. The mixture was added ice-water (10 mL), and extracted with EA (20 mL*2). The combined organic layers were washed with brine (20 mL), dried over Na₂SO₄, filtered and concentrated. The residue was purified by pre-HPLC to afford 587 as a white solid (72 mg, 45%). MS (ESI) m/z 415 [M+H]⁺. ¹H NMR (400 MHz, CD₃OD) δ 7.82-7.73 (m, 1H), 7.56-7.45 (m, 3H), 6.80-6.75 (m, 1H), 5.00 (dd, J=9.0, 5.7 Hz, 1H), 4.14 (dd, J=14.2, 7.6 Hz, 1H), 3.66-3.57 (m, 1H), 3.60 (s, 3H), 3.47 (dd, J=14.2, 6.0 Hz, 1H), 2.89 (dd, J=14.8, 9.0 Hz, 1H), 2.80-2.57 (m, 3H), 2.49-2.32 (m, 2H).

### Step 4. (2s,4R)-2-(((S)-1-((4-chloro-1-methyl-1H-pyrazol-5-yl)methyl)-3-oxoisoindolin-2-yl)methyl)-5-oxa-7-azaspiro[3.4]octane-6,8-dione (569) and (2s,4S)-2-(((R)-1-((4-chloro-1-methyl-1H-pyrazol-5-yl)methyl)-3-oxoisoindolin-2-yl)methyl)-5-oxa-7-azaspiro[3.4]octane-6,8-dione (644)

Chiral separation of compound 587 by chiral prep-HPLC (IE: Hex-EtOH-TFA=40-60-0.2) was conducted to afford two isomers: compounds 569 and 644. One of the two isomers was a white solid (20 mg, 19%), with RT=9.799 min (IE: Hex-EtOH-TFA=40-60-0.2), MS (ESI) m/z 415 [M+H]⁺. ¹H NMR (400 MHz, CD₃OD) δ 7.81-7.74 (m, 1H), 7.53-7.48 (m, 3H), 6.81-6.75 (m, 1H), 5.01 (dd, J=9.0, 5.7 Hz, 1H), 4.14 (dd, J=14.4, 7.6 Hz, 1H), 3.61 (s, 3H), 3.67-3.57 (m, 1H), 3.48 (dd, J=14.3, 6.0 Hz, 1H), 2.90 (dd, J=15.2, 9.2 Hz, 1H), 2.79-2.60 (m, 3H), 2.50-2.34 (m, 2H). The other one of the two isomers was a white solid (16 mg, 15.2%), with RT=8.013 min (IE: Hex-EtOH-TFA=40-60-0.2), MS (ESI) m/z 415 [M+H]⁺. ¹H NMR (400 MHz, CD₃OD) δ 7.80-7.75 (m, 1H), 7.54-7.47 (m, 3H), 6.81-6.75 (m, 1H), 5.04-4.98 (m, IH), 4.14 (dd, J=14.0, 7.6 Hz, 1H), 3.64-3.59 (m, 4H), 3.51-3.46 (m, 1H), 2.93-2.86 (m, 1H), 2.77-2.61 (m, 3H), 2.50-2.36 (m, 2H).

### (R,E)-3-((4-chloro-1-methyl-1H-pyrazol-5-yl)methyl)-2-(3-(5-fluoro-1H-1,2,3-triazol-4-yl)allyl)isoindolin-1-one (570) and (S,E)-3-((4-chloro-1-methyl-1H-pyrazol-5-yl)methyl)-2-(3-(5-fluoro-1H-1,2,3-triazol-4-yl)allyl)isoindolin-1-one (571)

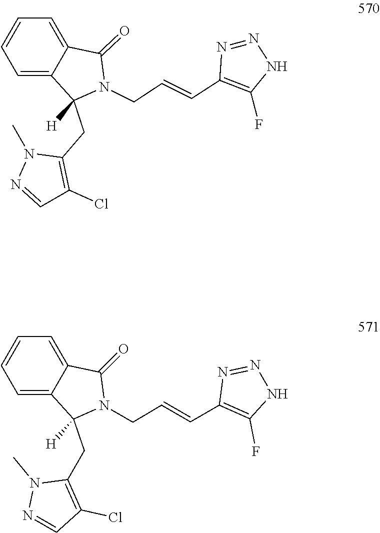 [SMILES: Cn1ncc(Cl)c1C[C@@H]1c2ccccc2C(=O)N1C/C=C/c1nn[nH]c1F ; Cn1ncc(Cl)c1C[C@H]1c2ccccc2C(=O)N1C/C=C/c1nn[nH]c1F]

Chiral separation of compound 516 by chiral prep-HPLC (IA: 250 mm*4.6 mm 5 um, Hex-EtOH-TFA=60-40-0.2) was conducted to afford two enantiomers: compounds 570 and 571. One of the two enantiomers was a white solid, with RT=5.999 min (IA: 250 mm*4.6 mm Sum, Hex-EtOH-TFA=60-40-0.2). MS (ESI) m/z 387 [M+H]⁺. ¹H NMR (400 MHz, CD₃OD) δ 7.83-7.81 (m, 1H), 7.58-7.49 (m, 3H), 6.89-6.86 (m, 1H), 6.52 (d, J=16.4 Hz, 1H), 6.38-6.31 (m, 1H), 5.03 (dd, J=6.0, 8.4 Hz, 1H), 4.87 (dd, J=5.0, 1.8 Hz, 1H), 4.05 (dd, J=16.0, 7.6 Hz, 1H), 3.61 (s, 3H), 3.56 (dd, J=14.8, 6.1 Hz, 1H), 2.97 (dd, J=14.8, 8.8 Hz, 1H). The other one of the two enantiomers was a white solid, with RT=7.223 min (IA: 250 mm*4.6 mm Sum, Hex-EtOH-TFA=60-40-0.2). MS (ESI) m/z 387 [M+H]⁺. ¹H NMR (400 MHz, CD₃OD) δ 7.83-7.81 (m, 1H), 7.58-7.49 (m, 3H), 6.89-6.86 (m, 1H), 6.52 (d, J=16.4 Hz, 1H), 6.38-6.31 (m, 1H), 5.03 (dd, J=6.0, 8.4 Hz, 1H), 4.87 (dd, J=5.0, 1.8 Hz, 1H), 4.05 (dd, J=16.0, 7.6 Hz, 1H), 3.61 (s, 3H), 3.56 (dd, J=14.8, 6.1 Hz, 1H), 2.97 (dd, J=14.8, 8.8 Hz, 1H).

### 3-((4-chloro-1-methyl-1H-pyrazol-5-yl)methyl)-2-(4-fluorobenzyl)isoindolin-1-one (572)

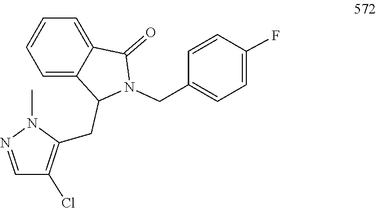 [SMILES: Cn1ncc(Cl)c1CC1c2ccccc2C(=O)N1Cc1ccc(F)cc1]

The title compound 572 was prepared according to the procedure described for compound 502 as a white solid (44 mg, 26.10%), MS (ESI) m/z 370 [M+H]⁺. ¹H NMR (400 MHz, DMSO-d₆) δ 7.78-7.72 (m, 1H), 7.55 (s, 1H), 7.54-7.49 (m, 2H), 7.30-7.23 (m, 2H), 7.19-7.13 (m, 2H) 6.89-6.83 (m, 11H), 5.19 (d, J=15.2 Hz, 1H), 4.66 (dd, J=8.0, 6.0 Hz, 1H), 4.32 (d, J=15.2 Hz, 1H), 3.57 (s, 3H), 3.48 (dd, J=14.8, 6.0 Hz, 1H), 2.99 (dd, J=14.8, 8.2 Hz, 1H).

### 3-((4-chloro-1-methyl-1H-pyrazol-5-yl)methyl)-2-(4-chlorobenzyl)isoindolin-1-one (573)

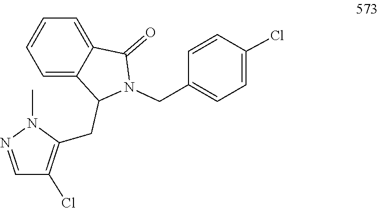 [SMILES: Cn1ncc(Cl)c1CC1c2ccccc2C(=O)N1Cc1ccc(Cl)cc1]

The title compound 573 was prepared according to the procedure described for compound 502 as a white solid (26 mg, 27.1%), MS (ESI) m/z 386 [M+H]⁺. ¹H NMR (400 MHz, DMSO-d₆) δ 7.78-7.73 (m, 1H), 7.55-7.50 (m, 3H), 7.43-7.34 (m, 2H), 7.25-7.21 (m, 2H), 6.90-6.84 (m, 1H), 5.17 (d, J=15.6 Hz, 1H), 4.67 (dd, J=8.0, 6.0 Hz, 1H), 4.33 (d, J=15.6 Hz, 1H), 3.57 (s, 3H), 3.47 (dd, J=14.9, 6.0 Hz, 1H), 2.98 (dd, J=14.8, 8.4 Hz, 1H).

### 3-((4-chloro-1-methyl-1H-pyrazol-5-yl)methyl)-2-((6-methoxypyridin-3-yl)methyl)isoindolin-1-one (574)

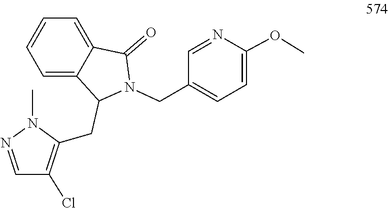 [SMILES: COc1ccc(CN2C(=O)c3ccccc3C2Cc2c(Cl)cnn2C)cn1]

The title compound 574 was prepared according to the procedure described for compound 502 as a white solid (43 mg, 29.3%), MS (ESI) m/z 383 [M+H]⁺. ¹H NMR (400 MHz, DMSO-d₆) δ 8.07 (d, J=2.5 Hz, 1H), 7.76-7.68 (m, 1H), 7.59-7.45 (m, 4H), 6.90-6.82 (m, 1H), 6.78 (d, J=8.5 Hz, 11H), 5.13 (d, J=15.2 Hz, 1H), 4.69 (dd, J=8.3, 5.8 Hz, 1H), 4.29 (d, J=15.3 Hz, 1H), 3.82 (s, 3H), 3.60 (s, 3H), 3.52 (dd, J=14.9, 5.9 Hz, 1H), 2.98 (dd, J=14.8, 8.3 Hz, 1H).

### 2-(4-bromobenzyl)-3-((4-chloro-1-methyl-1H-pyrazol-5-yl)methyl)isoindolin-1-one (575)

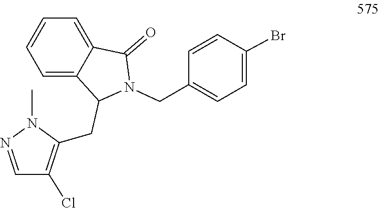 [SMILES: Cn1ncc(Cl)c1CC1c2ccccc2C(=O)N1Cc1ccc(Br)cc1]

The title compound 575 was prepared according to the procedure described for compound 502 as a white solid (35 mg, 32.7%). MS (ESI) m/z 430 [M+H]⁺. ¹H NMR (400 MHz, DMSO-d₆) δ 7.77-7.72 (m, L H), 7.57-7.47 (m, 5H), 7.20-7.14 (m, 2H), 6.90-6.84 (m, 1H), 5.15 (d, J=15.6 Hz, 1H), 4.68 (dd, J=8.0, 6.0 Hz, 11H), 4.32 (d, J=15.6 Hz, 1H), 3.57 (s, 3H), 3.47 (dd, J=14.8, 6.0 Hz, 1H), 2.98 (dd, J=14.8, 8.4 Hz, 1H).

### (S,E)-3-((4-chloro-1-methyl-1H-pyrazol-5-yl)methyl)-2-(3-(1-methyl-1H-1,2,3-triazol-4-yl)allyl)isoindolin-1-one (576-1), (S,E)-3-((4-chloro-1-methyl-1H-pyrazol-5-yl)methyl)-2-(3-(1-methyl-1H-1,2,3-triazol-5-yl)allyl)isoindolin-1-one (576-2) and (S,E)-3-((4-chloro-1-methyl-1H-pyrazol-5-yl)methyl)-2-(3-(2-methyl-2H-1,2,3-triazol-4-yl)allyl)isoindolin-1-one (577)

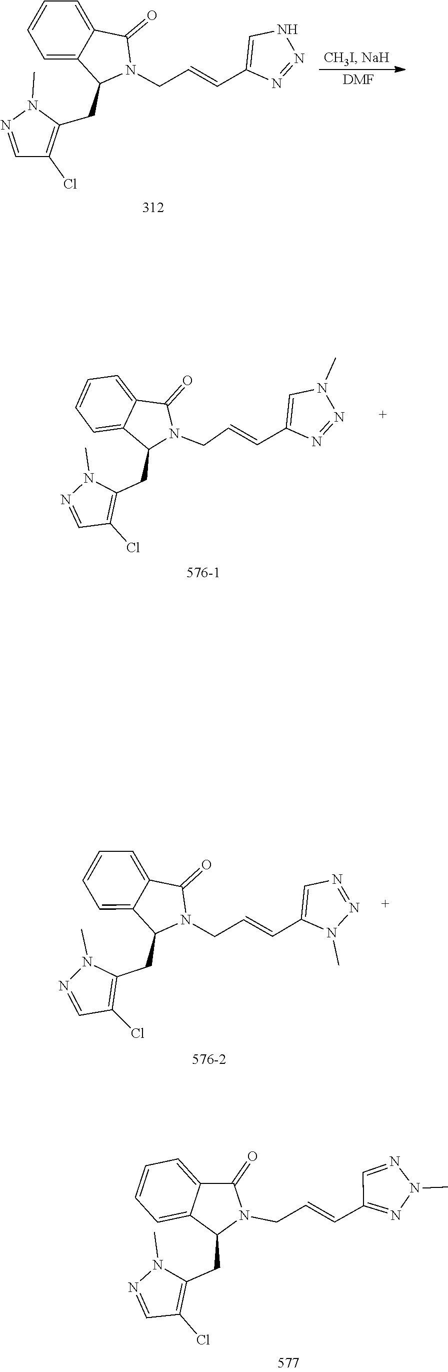

To a solution of 312 (130 mg, 0.35 mmol) in DMF (5 mL) was added NaH (14 mg, 0.35 mmol) at 0° C. under N₂. The mixture was stirred for 30 min. CH₃I (100 mg, 0.71 mmol) was added and the reaction mixture was warmed to r.t. This suspension was stirred for an additional 2 h. Water was added to the suspension at 0° C. and the suspension was extracted with EA. The organic layer was separated, dried over anhydrous Na₂SO₄ and concentrated under vacuum. The residue was purified by prep-HPLC to give two products: a mixture of two isomers of 576-1, 576-2 and 577, and the remaining one of 576-1, 576-2 and 577. The mixture of two isomers of 576-1, 576-2 and 577 was a white solid (30 mg, 22.2%), with RT=1.11 min (Column: xBridge®BEHC18 C18+, 2.5 um 4.6*30 mm, column XP; Mobile phase: ACN-Water (0.1% FA); Gradient: 5% ACN to 95% ACN in 0.8 min, hold 0.8 min, 95% ACN to 5% ACN in 1.4 min, total 3 min; Flow rate:1.8 mL/min; Column Temp: 40 degree). MS (ESI) m/z 383 [M+H]⁺. ¹H NMR (400 MHz, DMSO-d) δ 8.11 (s, 0.7H), 7.91 (s, 0.3H), 7.76-7.68 (m, 1H), 7.60-7.47 (m, 3H), 6.96-6.88 (m, 1H), 6.51 (d, J=16.5 Hz, 11H), 6.42-6.19 (m, 1H), 5.01-4.88 (m, 11H), 4.81-4.64 (m, 1H), 4.00 (s, 3H), 3.94 (dd, J=15.9, 8.0 Hz, 1H), 3.65 (s, 0.9H), 3.64 (s, 2.1H), 3.56-3.40 (m, 1H), 3.10-2.90 (m, 1H). The remaining one of 576-1, 576-2 and 577 was a white solid (20 mg, 14.8%). With RT=1.19 min, (Column: xBridge®BEHC18 C18+, 2.5 um 4.6*30 mm, column XP; Mobile phase: ACN-Water (0.1% FA); Gradient: 5% ACN to 95% ACN in 0.8 min, hold 0.8 min, 95% ACN to 5% ACN in 1.4 min, total 3 min; Flow rate:1.8 mL/min; Column Temp: 40 degree). MS (ESI) m/z 383 [M+H]⁺. ¹H NMR (400 MHz, DMSO-d₆) δ 7.88 (s, 1H), 7.75-7.68 (m, 1H), 7.59-7.47 (m, 3H), 6.94-6.88 (m, 1H), 6.50 (d, J=16.1 Hz, 1H), 6.38-6.26 (m, 1H), 4.92 (dd, J=8.4, 6.0 Hz, 11H), 4.75-4.66 (m, 1H), 4.09 (s, 3H), 3.94 (dd, J=16.0, 7.8 Hz, 1H), 3.64 (s, 3H), 3.49 (dd, J=14.8, 6.0 Hz, 1H), 2.98 (dd, J=14.8, 8.4 Hz, 1H).

### 3-[(4-chloro-2-methyl-pyrazol-3-yl)methyl]-2-[(6-hydroxy-3-pyridyl)methyl]isoindolin-1-one (578)

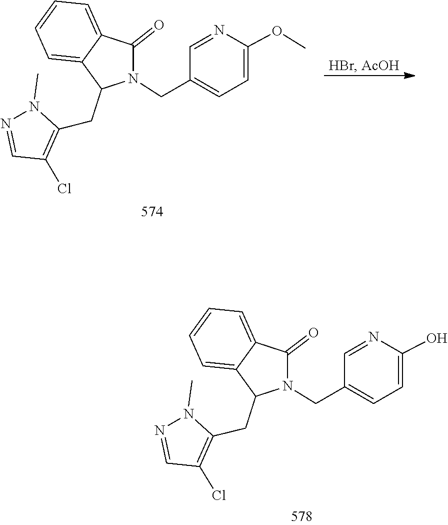 [SMILES: COc1ccc(CN2C(=O)c3ccccc3C2Cc2c(Cl)cnn2C)cn1 ; Cn1ncc(Cl)c1CC1c2ccccc2C(=O)N1Cc1ccc(O)nc1]

A mixture of 574 (20 mg, 0.052 mmol) in HBr/AcOH (5 mL) was stirred for 4 h at 100° C.C. After the reaction was completed, the mixture was concentrated. The residue was purified by prep-HPLC to give 578 (7 mg, 0.019 mmol) as a white solid. MS (ESI) m/z 369 [M+H]⁺. ¹H NMR (400 MHz, DMSO-d₆) δ 11.54 (s, 1H), 7.75-7.68 (m, 1H), 7.55 (s, 1H), 7.53-7.48 (m, 2H), 7.34 (d, J=2.6 Hz, 1H), 7.26 (dd, J=9.4, 2.8 Hz, 1H), 6.83-6.76 (m, 1H), 6.28 (d, J=9.4 Hz, 1H), 4.93 (d, J=15.2 Hz, 1H), 4.70 (dd, J=8.6, 5.6 Hz, 1H), 4.10 (d, J=15.2 Hz, 1H), 3.61 (s, 3H), 3.56 (dd, J=14.8, 5.6 Hz, 1H), 2.92 (dd, J=14.8, 8.8 Hz, 11H).

### (S)-3-((4-chloro-1-methyl-1H-pyrazol-5-yl)methyl)-2-(((R)-1-methyl-4,5,6,7-tetrahydro-1H-benzo[d][1,2,3]triazol-5-yl)methyl)isoindolin-1-one (579) and (S)-3-((4-chloro-1-methyl-1H-pyrazol-5-yl)methyl)-2-(((R)-1-methyl-4,5,6,7-tetrahydro-1H-benzo[d][1,2,3]triazol-6-yl)methyl)isoindolin-1-one (645)

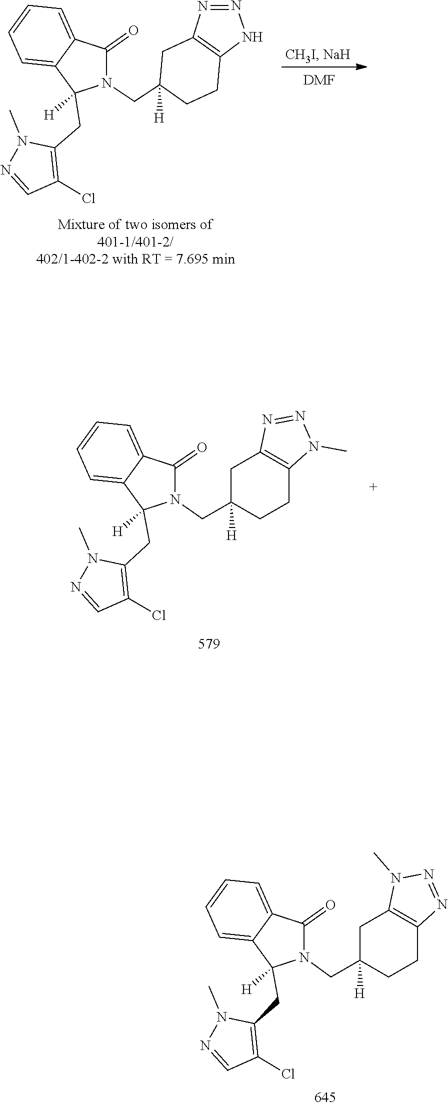

The title compounds 579 and 645 were prepared according to the procedure described for 393-2. After purification by pre-HPLC, two isomers were obtained: 579 and 645. One of the two isomers was a white solid, with RT=1.11 min (Column: xBridge@BEHC18 C18+, 2.5 um 4.6*30 mm, column XP; Mobile phase: ACN-Water (0.10% FA); Gradient: 5% ACN to 95% ACN in 0.8 min, hold 0.8 min, 95% ACN to 5% ACN in 1.4 min, total 3 min; Flow rate:1.8 mL/min; Column Temp: 40 degree). MS (ESI) m/z 411 [M+H]⁺. ¹H NMR (400 MHz, DMSO-d₆) δ 7.74-7.68 (m, 1H), 7.58-7.46 (m, 3H), 6.92-6.80 (m, 1H), 5.02 (p, J=6.7 Hz, 1H), 3.99-3.89 (m, 1H), 3.87-3.84 (m, 2H), 3.76-3.65 (m, 4H), 3.61-3.48 (m, 1H), 3.30-3.25 (m, 1H), 3.0-2.9 (m, 1H), 2.83-2.64 (m, 3H), 2.44-2.12 (m, 2H), 1.98-1.70 (m, 1H), 1.58-1.30 (m, 1H). The other one of the two isomers was a white solid, with RT=1.19 min (Column: xBridge@BEHC18 C18+, 2.5 um 4.6*30 mm, column XP; Mobile phase: ACN-Water (0.1% FA); Gradient: 5% ACN to 95% ACN in 0.8 min, hold 0.8 min, 95% ACN to 5% ACN in 1.4 min, total 3 min; Flow rate:1.8 mL/min; Column Temp: 40 degree). MS (ESI) m/z 411 [M+H]⁺. ¹H NMR (400 MHz, DMSO) δ 7.73-7.69 (m, 1H), 7.58-7.46 (m, 3H), 6.91-6.87 (m, 1H), 5.02 (q, J=7.7 Hz, 1H), 4.04-3.88 (m, 4H), 3.68 (s, 1.5H), 3.67 (s, 1.5H), 3.52 (dd, J=14.8, 6.0 Hz, 1H), 3.29-3.21 (m, 1H), 2.95 (dd, J=14.8, 8.3 Hz, 2H), 2.77-2.55 (m, 2H), 2.40-2.13 (m, 2H), 1.94-1.71 (m, 1H), 1.57-1.31 (m, 1H).

### 2-((5′-chloro-3′-((4-chloro-1-methyl-1H-pyrazol-5-yl)methyl)-7′-fluorospiro[cyclopropane-1,1′-isoindolin]-2′-yl)methyl)-5-oxa-7-azaspiro[3.4]octan-6-one (580)

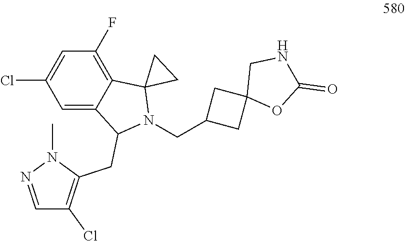 [SMILES: Cn1ncc(Cl)c1CC1c2cc(Cl)cc(F)c2C2(CC2)N1CC1CC2(CNC(=O)O2)C1]

The title compound 580 was prepared according to the procedure described for compound 270 as a white solid (3 mg, 3.8%). MS (ESI) m/z 465 [M+H]⁺. ¹H NMR (400 MHz, DMSO-d₆) δ 7.48 (s, 1H), 7.44 (s, 1H), 7.23 (dd, J=9.8, 1.6 Hz, 1H), 7.05 (d, J=1.6 Hz, 1H), 4.28 (dd, J=8.7, 5.3 Hz, 1H), 3.76 (s, 3H), 3.40 (d, J=8.9 Hz, 1H), 3.33-3.27 (m, 3H), 3.06-2.84 (m, 2H), 2.14-1.99 (m, 1H), 1.92-1.71 (m, 3H), 1.65-1.45 (m, 2H), 1.29-1.14 (m, 2H), 0.91-0.86 (m, 1H).

The intermediate 580-2 was prepared as follows:

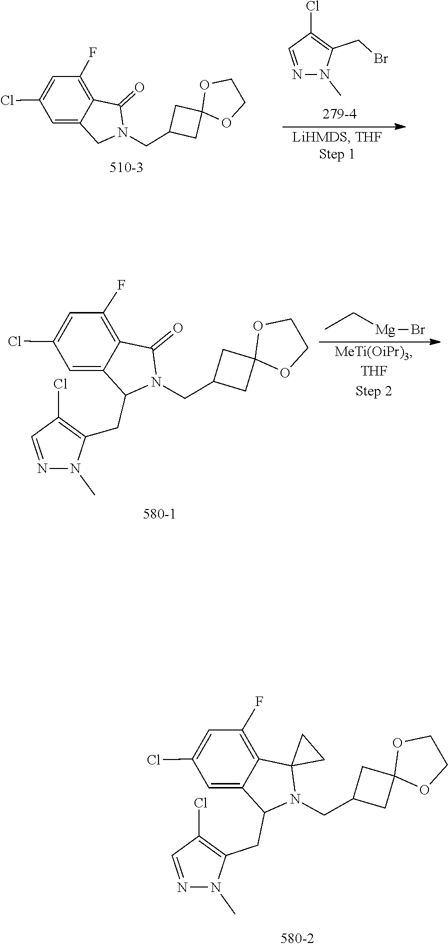

Step 1 was performed according to the procedure outlined for the preparation of Int 12-2.

Step 2 was performed according to the procedure outlined for the preparation of 539-2.

### 5-chloro-3-((4-chloro-1-methyl-1H-pyrazol-5-yl)methyl)-7-fluoro-2-(4-methoxybenzyl)isoindolin-1-one (581)

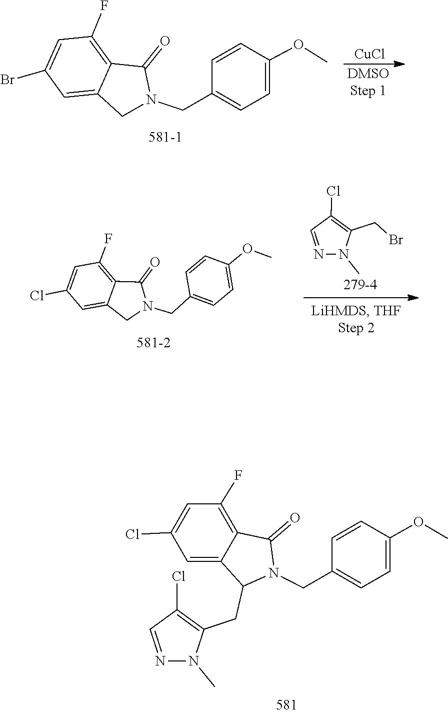

Step 1 was performed according to the procedure outlined for preparation of 474.

Step 2 was performed according to the procedure outlined for preparation of Int 12-2 to afford 581 (15 mg, 7.0%) as a white solid. MS (ESI) m/z 434 [M+H]+H NMR (400 MHz, DMSO-d₆) δ 7.56-7.53 (m, 2H), 7.14-7.06 (m, 2H), 6.93-6.85 (m, 3H), 5.10 (d, J=15.1 Hz, 1H), 4.64 (t, J=6.8 Hz, 1H), 4.12 (d, J=15.1 Hz, 1H), 3.72 (s, 3H), 3.61 (s, 3H), 3.43 (dd, J=15.2, 6.5 Hz, 1H), 3.12 (dd, J=15.0, 7.4 Hz, 1H).

### 2-((1-((4-chloro-1-methyl-1H-pyrazol-5-yl)methyl)-3-oxoisoindolin-2-yl)methyl)cyclopropane-1-carboxamide (582)

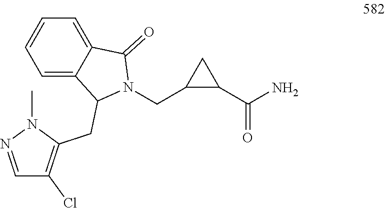 [SMILES: Cn1ncc(Cl)c1CC1c2ccccc2C(=O)N1CC1CC1C(N)=O]

The title compound 582 was prepared according to the procedure described for compound 404 as a white solid (20 mg, 34.9%). MS (ESI) m/z 359 [M+H]⁺. ¹H NMR (400 MHz, DMSO-d₆) δ 7.74-7.62 (m, 1H), 7.57-7.44 (m, 4H), 6.86-6.71 (m, 2H), 5.07-4.96 (m, 1H), 3.92-3.82 (m, 0.5H), 3.77-3.69 (m, 0.5H), 3.65 (s, 3H), 3.64-3.50 (m, 1H), 3.39-3.33 (m, 0.5H), 3.19-3.09 (m, 0.5H), 2.96-2.85 (m, 1H), 1.69-1.60 (m, 0.5H), 1.52-1.45 (m, 1H), 1.45-1.33 (m, 0.5H), 0.98-0.89 (m, 0.5H), 0.89-0.80 (m, 1H), 0.74-0.65 (m, 0.5H).

### 3-((2-(4-(methoxy-d₃)benzyl)-3-oxoisoindolin-1-yl)methyl)-4-methylpicolinonitrile (583)

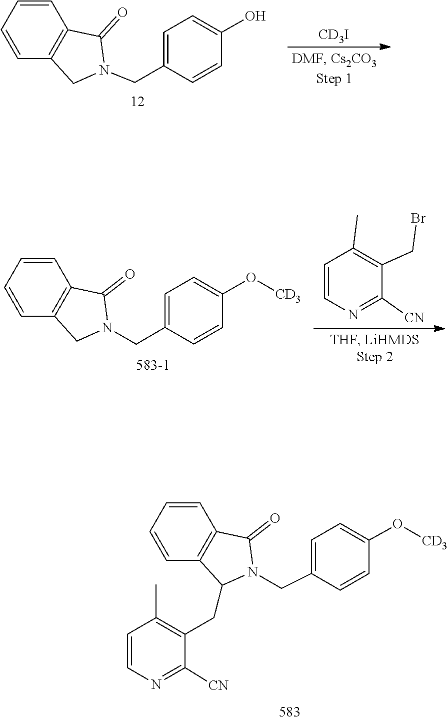

### Step 1. 2-(4-(methoxy-d₃)benzyl)isoindolin-1-one (583-1)

To a solution of 12 (100 mg, 0.41 mmol) in DMF (2 mL) were added Cs₂CO₃ (272 mg, 0.83 mmol) and iodomethane-d₃ (91 mg, 0.62 mmol). The mixture was stirred for 13 h, and then the mixture was diluted with water, and extracted with ethyl acetate. The organic layer was separated, dried over anhydrous Na₂SO₄ and concentrated to afford 583-1 (100 mg) as a yellow solid, which was used in the next step without further purification. MS (ESI) m/z 257 [M+H]⁺.

Step 2 was performed according to the procedure outlined for preparation of Int 12-2 to afford 583 (90 mg, 57%) as a yellow solid. MS (ESI) m/z 387 [M+H]⁺. ¹H NMR (400 MHz, DMSO-d₆) δ 8.57 (d, J=4.9 Hz, 1H), 7.78 (d, J=7.5 Hz, 1H), 7.61 (d, J=4.9 Hz, 1H), 7.59-7.48 (m, 11H), 7.48-7.40 (m, 11H), 7.20-7.12 (m, 2H), 6.93-6.84 (m, 2H), 6.47 (d, J=7.5 Hz, 1H), 5.15 (d, J=15.2 Hz, 11H), 4.69 (dd, J=9.4, 6.1 Hz, 1H), 4.26 (d, J=15.2 Hz, 11H), 3.76 (dd, J=14.0, 6.1 Hz, 11H), 2.82 (dd, J=13.9, 9.4 Hz, 11H), 2.22 (s, 3H).

### 2-(4-acetylbenzyl)-3-((4-chloro-1-methyl-1H-pyrazol-5-yl)methyl)isoindolin-1-one (584)

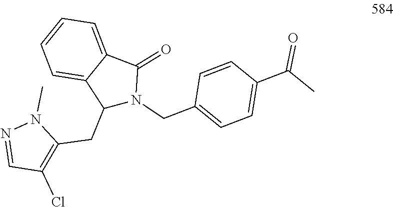 [SMILES: CC(=O)c1ccc(CN2C(=O)c3ccccc3C2Cc2c(Cl)cnn2C)cc1]

The title compound 584 was prepared according to the procedure described for compound 502 as a white solid (33 mg, 33.2%), MS (ESI) m/z 394 [M+H]⁺. ¹H NMR (400 MHz, DMSO-d₆) δ 7.93 (d, J=8.3 Hz, 2H), 7.78-7.74 (m, 1H), 7.58-7.47 (m, 3H), 7.35 (d, J=8.0 Hz, 2H), 6.93-6.85 (m, 11H), 5.25 (d, J=15.6 Hz, 1H), 4.70 (dd, J=8.0, 6.0 Hz, 1H), 4.45 (d, J=15.6 Hz, 1H), 3.57 (s, 3H), 3.48 (dd, J=14.8, 6.0 Hz, 1H), 3.00 (dd, J=14.8, 8.4 Hz, 1H), 2.55 (s, 3H).

### 3-((4-chloro-1-methyl-1H-pyrazol-5-yl)methyl)-2-((1-methyl-1H-benzo[d]imidazol-5-yl)methyl)isoindolin-1-one (585)

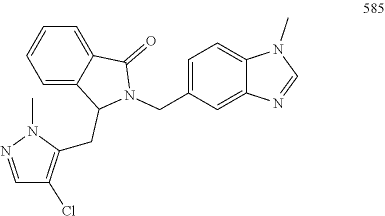 [SMILES: Cn1ncc(Cl)c1CC1c2ccccc2C(=O)N1Cc1ccc2c(c1)ncn2C]

The title compound 585 was prepared according to the procedure described for compound 502 as a white solid (10 mg, 21%). MS (ESI) m/z 406 [M+H]⁺. ¹H NMR (400 MHz, CDCl₃) δ 7.99 (s, 1H), 7.92 (d, J=7.5 Hz, 1H), 7.73 (s, 1H), 7.56-7.26 (m, 5H), 6.56 (d, J=7.5 Hz, 1H), 5.53 (d, J=15.1 Hz, 1H), 4.66 (dd, J=9.3, 5.6 Hz, 1H), 4.39 (d, J=15.0 Hz, 1H), 3.86 (s, 3H), 3.52-3.28 (m, 4H), 2.66 (dd, J=14.4, 9.5 Hz, 1H).

The intermediate 585-2 was prepared as follows:

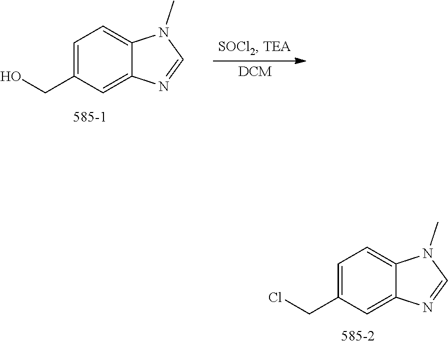

To a solution of (1-methyl-1H-benzo[d]imidazol-5-yl)methanol (162 mg, 1 mmol) in dried DCM (5 mL) were added SOCl₂ (2 mL) and TEA (242 mg, 2 mmol) at 0° C. under N₂. The mixture was stirred at 25° C. for 1 h, then quenched with water (15 mL) and extracted with ethyl acetate (10 mL*2). The combined organic layer was washed with water (10 mL) and brine (10 mL), dried 15 over Na₂SO₄, filtered, and concentrated in vacuo. The residue was purified by column chromatography on silica gel (eluting PE/EA=3/1 to 2/1) to 585-2 (170 mg, 98%) as a yellow oil. MS (ESI) m/z 181 [M+H]⁺.

### 3-((4-chloro-1-methyl-1H-pyrazol-5-yl)methyl)-2-((1-methyl-1H-indazol-5-yl)methyl)isoindolin-1-one (586)

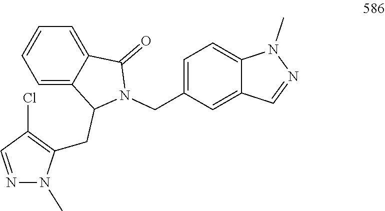 [SMILES: Cn1ncc(Cl)c1CC1c2ccccc2C(=O)N1Cc1ccc2c(cnn2C)c1]

The title compound 586 was prepared according to the procedure described for compound 502 as a white solid (10 mg, 11%). MS (ESI) m/z 406 [M+H]¹H NMR (400 MHz, CDCl₃) δ 7.95-7.91 (m, 2H), 7.59 (s, 1H), 7.51-7.46 (m, 2H), 7.41 (td, J=7.5, 1.3 Hz, 1H), 7.38-7.26 (m, 2H), 6.60 (d, J=7.5 Hz, 1H), 5.52 (d, J=15.0 Hz, 1H), 4.63 (dd, J=9.0, 6.2 Hz, 1H), 4.30 (d, J=15.0 Hz, 1H), 4.06 (s, 3H), 3.42 (s, 3H), 3.42 (dd, J=14.5, 6.1 Hz, 1H), 2.68 (dd, J=14.5, 9.1 Hz, 1H).

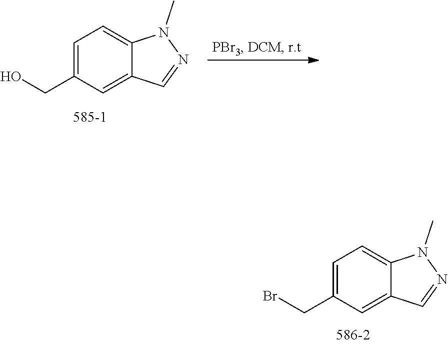

586-2 was prepared according to the procedure outlined for the preparation of 217-3.

### 3-((2-((6-methoxypyridin-3-yl)methyl)-3-oxoisoindolin-1-yl)methyl)-4-methylpicolinonitrile (588)

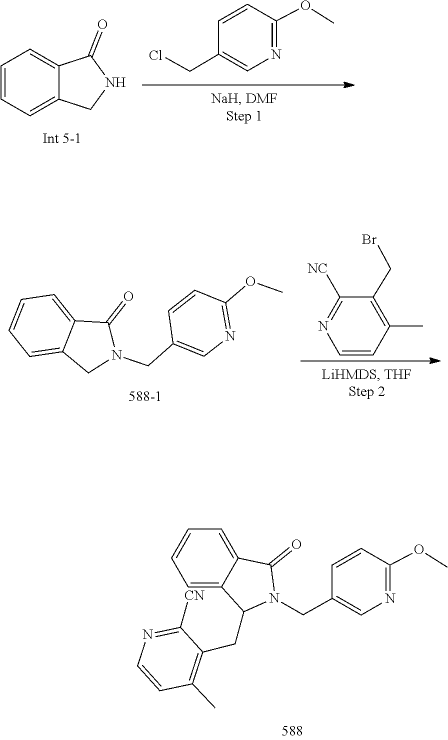

Step 1 was performed according to the procedure outlined for preparation of Int 5.

Step 2 was performed according to the procedure outlined for preparation of Int 12-2 to afford 588 (77 mg, 42.7%) as a white solid. MS (ESI) m/z 385 [M+H]+¹H NMR (400 MHz, CDCl₃) δ 8.56 (d, J=4.9 Hz, 1H), 8.10 (d, J=2.5 Hz, 1H), 7.92 (d, J=7.6 Hz, 1H), 7.62 (dd, J=8.6, 2.5 Hz, 1H), 7.49 (t, J=7.5 Hz, 1H), 7.40-7.32 (m, 2H), 6.73 (d, J=8.5 Hz, 1H), 6.46 (d, J=7.3 Hz, 1H), 5.21 (d, J=15.3 Hz, 1H), 4.80 (dd, J=9.5, 5.9 Hz, 1H), 4.36 (d, J=15.3 Hz, 1H), 3.94 (s, 3H), 3.63 (dd, J=13.8, 6.0 Hz, 1H), 2.85 (dd, J=13.8, 9.5 Hz, 1H), 2.05 (s, 3H).

### 3-((3-chloro-1-methyl-6-oxo-1,6-dihydropyridin-2-yl)methyl)-2-(4-methoxybenzyl)isoindolin-1-one (589)

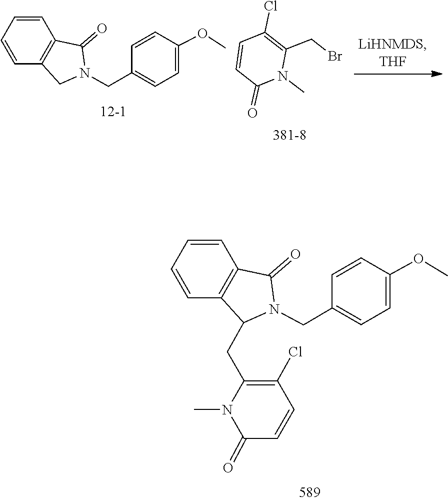

The title compound 589 was prepared according to the procedure described for Int 12-2 to afford 589 (40 mg, 10%) as a white solid. MS (ESI) m/z 409 [M+H]⁺. ¹H NMR (400 MHz, CDCl₃) δ 7.93 (d, J=7.5 Hz, 1H), 7.50 (t, J=7.3 Hz, 1H), 7.43 (td, J=7.5, 1.2 Hz, 1H), 7.34 (d, J=9.7 Hz, 1H), 7.20-7.15 (m, 2H), 6.88-6.82 (m, 2H), 6.74 (d, J=7.6 Hz, 1H), 6.62 (d, J=9.7 Hz, 1H), 5.38 (d, J=15.1 Hz, 1H), 4.77 (dd, J=9.0, 6.8 Hz, 1H), 4.15 (d, J=15.1 Hz, 1H), 3.79 (s, 3H), 3.59 (dd, J=14.2, 6.6 Hz, 1H), 3.12 (s, 3H), 2.86 (dd, J=14.1, 9.1 Hz, 1H).

### 3-((2-(4-methoxybenzyl)-3-oxoisoindolin-1-yl)methyl)pyridine-2,4-dicarbonitrile (590)

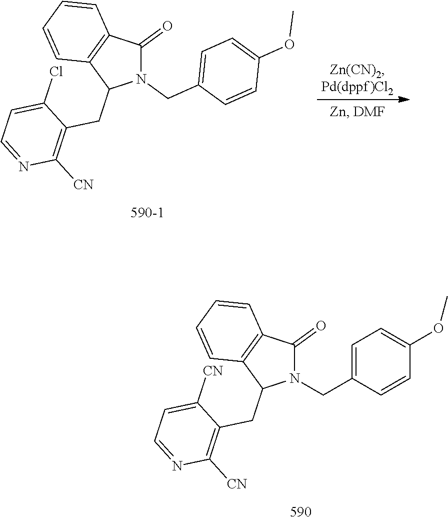 [SMILES: COc1ccc(CN2C(=O)c3ccccc3C2Cc2c(Cl)ccnc2C#N)cc1 ; COc1ccc(CN2C(=O)c3ccccc3C2Cc2c(C#N)ccnc2C#N)cc1]

The intermediate 590-1 was prepared according to the procedure outlined for 589. The title compound 590 was prepared according to the procedure described for 284-2 as a white solid (27 mg, 47%). MS (EST) m/z 395 [M+H]⁺. ¹H NMR (400 MHz, CDCl₃) δ 8.81 (d, J=4.9 Hz, 1H), 7.91 (d, J=7.6 Hz, 1H), 7.72 (d, J=4.9 Hz, 1H), 7.50 (t, J=7.6 Hz, 1H), 7.42 (td, J=7.5, 1.2 Hz, 1H), 7.24-7.16 (m, 2H), 6.85-6.80 (m, 2H), 6.62 (dd, J=7.6, 0.9 Hz, 1H), 5.22 (d, J=15.3 Hz, 1H), 4.88 (dd, J=7.9, 5.9 Hz, 1H), 4.47 (d, J=15.3 Hz, 1H), 3.78 (s, 3H), 3.78 (dd, J=14.0, 5.8 Hz, 1H), 3.12 (dd, J=14.0, 8.0 Hz, 1H).

### 3-((4-chloro-1-methyl-1H-pyrazol-5-yl)methyl)-2-((2-methoxythiazol-5-yl)methyl)isoindolin-1-one (591) and 5-((1-((4-chloro-1-methyl-1H-pyrazol-5-yl)methyl)-3-oxoisoindolin-2-yl)methyl)thiazol-2(3H)-one (592)

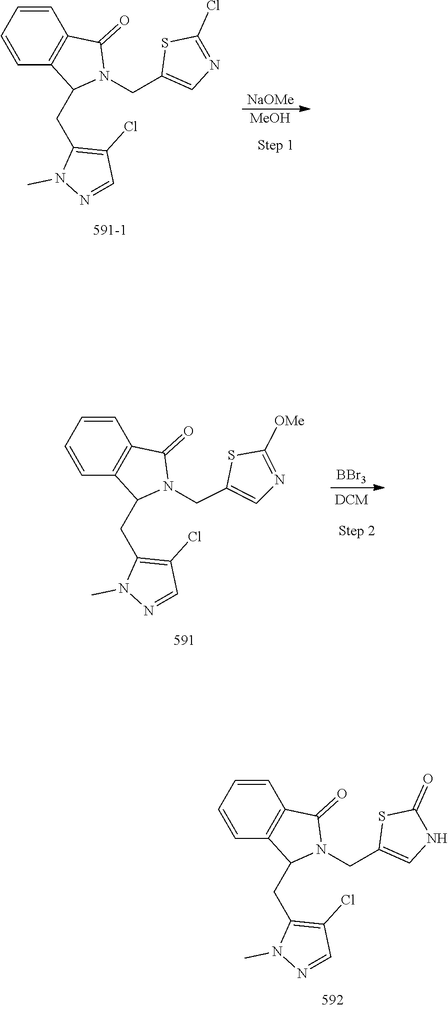

591-1 was prepared according to the procedure described for compound 502.

### Step 1. 3-((4-chloro-1-methyl-1H-pyrazol-5-yl)methyl)-2-((2-methoxythiazol-5-yl)methyl)isoindolin-1-one (591)

To a solution of 591-1 (100 mg, 0.25 mmol) in MeOH (2 mL) was added NaOMe (27 mg, 0.50 mmol) at r.t. The mixture was refluxed for 16 h. The mixture was concentrated and purified by HPLC to give 591 (14 mg, 14.4%) as a white solid. MS (ESI) m/z 389 [M+H]⁺. ¹H NMR (400 MHz, CD₃OD) δ 7.81-7.79 (m, 1H), 7.54-7.51 (m, 3H), 7.06 (s, 1H), 6.89-6.87 (m, 1H), 5.25 (d, J=15.6 Hz, 1H), 4.87 (dd, J=8.4, 6.3 Hz, 1H), 4.49 (d, J=15.6 Hz, 1H), 4.01 (s, 3H), 3.61 (s, 3H), 3.56 (dd, J=14.8, 6.4 Hz, 1H), 3.00 (dd, J=14.8, 8.0 Hz, 1H).

Step 2 was prepared according to the procedure described for step 2 of preparation of 14 to give 592 (19 mg, 21%) as a white solid. MS (ESI) m/z 375 [M+H]⁺. ¹H NMR (400 MHz, DMSO-d₆) δ 10.70 (brs, 1H), 7.71 (d, J=6.8 Hz, 1H), 7.56-7.49 (m, 3H), 6.86-6.85 (m, 2H), 4.95 (d, J=15.6 Hz, 1H), 4.80 (dd, J=8.4, 6.0 Hz, 1H), 4.31 (d, J=15.6 Hz, 1H), 3.65 (s, 3H), 3.57 (dd, J=14.8, 5.6 Hz, 1H), 2.96 (dd, J=14.4, 8.4 Hz, 1H).

### 3-((4-chloro-1-methyl-1H-pyrazol-5-yl)methyl)-2-((3-methoxyisoxazol-5-yl)methyl)isoindolin-1-one (593) and 5-((1-((4-chloro-1-methyl-1H-pyrazol-5-yl)methyl)-3-oxoisoindolin-2-yl)methyl)isoxazol-3(2H)-one (602)

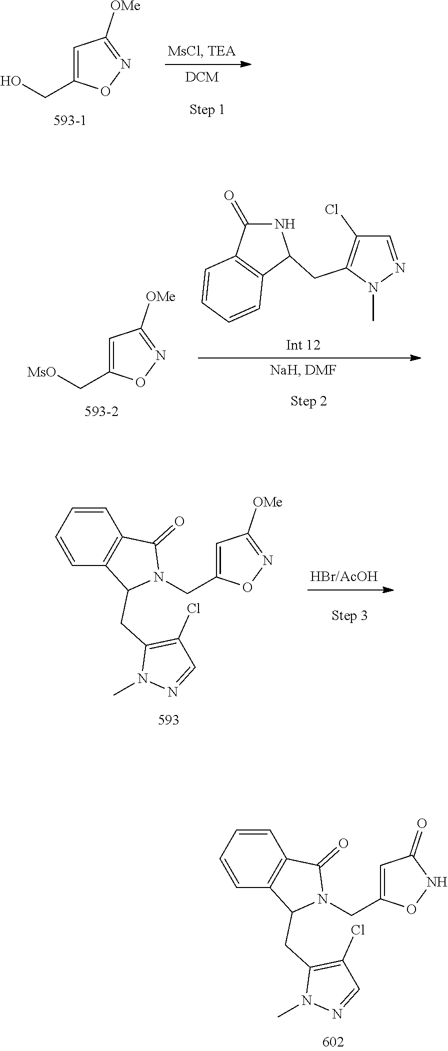 [SMILES: COc1cc(CO)on1 ; COc1cc(COS(C)(=O)=O)on1 ; Cn1ncc(Cl)c1CC1NC(=O)c2ccccc21 ; COc1cc(CN2C(=O)c3ccccc3C2Cc2c(Cl)cnn2C)on1 ; Cn1ncc(Cl)c1CC1c2ccccc2C(=O)N1Cc1cc(=O)[nH]o1]

Step 1 was performed according to the procedure outlined for the preparation of 132-2.

Step 2 was performed according to the procedure outlined for the preparation of Int 5 to afford 593 (250 mg, 80%) as a white solid. MS (ESI) m/z 373 [M+H]⁺. ¹H NMR (400 MHz, CD₃OD) δ 7.85-7.78 (m, 1H), 7.57-7.50 (m, 3H), 6.88-6.85 (m, 1H), 6.03 (s, 1H), 5.22 (d, J=16.4 Hz, 1H), 4.96 (dd, J=8.4, 5.6 Hz, 1H), 4.55 (d, J=16.8 Hz, 1H), 3.91 (s, 3H), 3.63 (s, 3H), 3.57 (dd, J=14.8, 6.0 Hz, 1H), 3.00 (dd, J=14.8, 8.8 Hz, 1H).

### Step 3. 5-((1-((4-chloro-1-methyl-1H-pyrazol-5-yl)methyl)-3-oxoisoindolin-2-yl)methyl)isoxazol-3(2H)-one (602)

To a solution of 593 (100 mg, 0.27 mmol) in AcOH (1 mL) was added HBr (1 mL, 40% in water) at r.t. The mixture was stirred at 80° C. for 6 h. The mixture was cooled to r.t., diluted with DCM (5 mL), and poured into water (2 mL), and the pH of the mixture was adjusted to 8-9 with sat. NaHCO₃ (aq.). The organic layer was concentrated under vacuum. The residue was purified by prep-HPLC to give 602 (21 mg, 21.6%) as a white solid. MS (ESI) m/z 359 [M+H]⁺. ¹H NMR (400 MHz, CD₃OD) δ 7.81-7.79 (m, 1H), 7.53-7.48 (m, 3H), 6.84-6.81 (m, 1H), 5.67 (s, 1H), 5.16 (d, J=16.0 Hz, 1H), 4.93 (dd, J=9.2, 4.4 Hz, 1H), 4.42 (d, J=15.6 Hz, 1H), 3.64 (s, 3H), 3.59 (dd, J=14.8, 5.6 Hz, 1H), 2.94 (dd, J=15.2, 9.2 Hz, 1H).

### 3-((4-chloro-1-methyl-1H-pyrazol-5-yl)methyl)-2-((3-methylbenzo[d]isoxazol-6-yl)methyl)isoindolin-1-one (594)

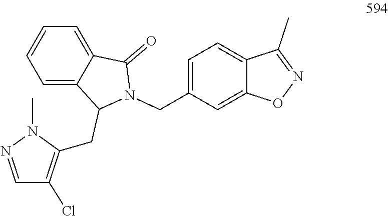 [SMILES: Cc1noc2cc(CN3C(=O)c4ccccc4C3Cc3c(Cl)cnn3C)ccc12]

The title compound 594 was prepared according to the procedure described for compound 502 as a white solid (31 mg, 24.6%). MS (ESI) m/z 407 [M+H]⁺. ¹H NMR (400 MHz, DMSO-d₆) δ 7.81 (d, J=8.0 Hz, 1H), 7.80-7.73 (m, 1H), 7.59-7.47 (m, 4H), 7.23 (dd, J=8.0, 1.2 Hz, 1H), 6.93-6.79 (m, 1H), 5.34 (d, J=15.4 Hz, 1H), 4.71 (dd, J=8.4, 6.0 Hz, 1H), 4.54 (d, J=15.6 Hz, 1H), 3.56 (s, 3H), 3.52 (dd, J=14.8, 5.9 Hz, 1H), 3.00 (dd, J=14.8, 8.4 Hz, 1H), 2.53 (s, 3H).

The intermediate 594-4 was prepared as follows:

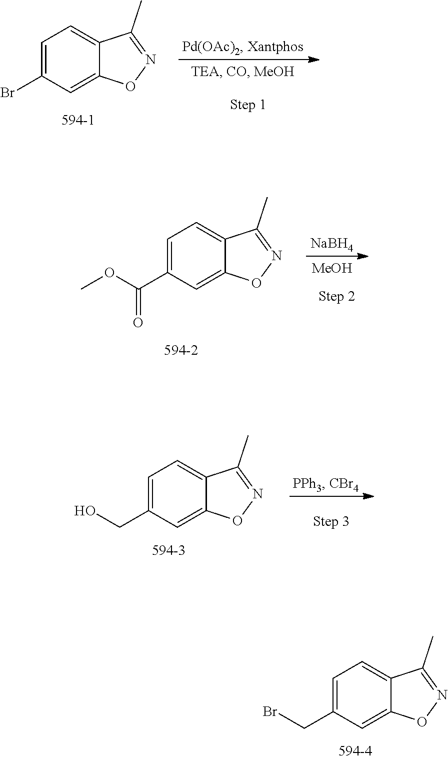

Step 1 was performed according to the procedure outlined for the preparation of 382-1.

Step 2 was performed according to the procedure outlined for the preparation of 164-2.

Step 3 was performed according to the procedure outlined for the preparation of 164-3.

### 5-((2-((6-methoxypyridin-3-yl)methyl)-3-oxoisoindolin-1-yl)methyl)-6-methylpyrimidine-4-carbonitrile (595)

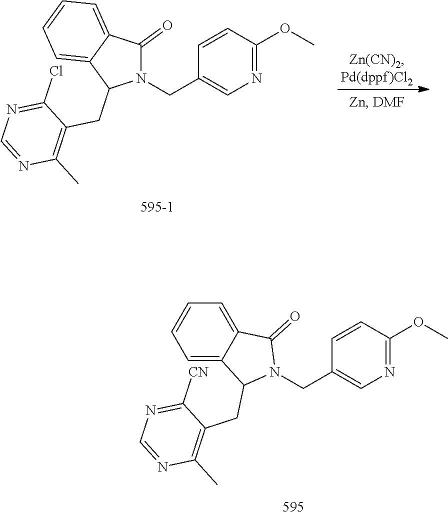 [SMILES: COc1ccc(CN2C(=O)c3ccccc3C2Cc2c(C)ncnc2Cl)cn1 ; COc1ccc(CN2C(=O)c3ccccc3C2Cc2c(C)ncnc2C#N)cn1]

The intermediate 595-1 was prepared according to the procedure outlined for 588. The title compound 595 was prepared according to the procedure described for 284-2 as a white solid (5 mg, 6.5%). MS (ESI) m/z 386 [M+H]⁺. ¹H NMR (400 MHz, CDCl₃) δ 9.15 (s, 1H), 8.12 (s, 1H), 7.94 (d, J=7.6 Hz, 1H), 7.59 (dd, J=8.5, 2.5 Hz, 1H), 7.51 (t, J=7.5 Hz, 1H), 7.40 (td, J=7.5, 1.2 Hz, 1H), 6.72 (d, J=8.5 Hz, 1H), 6.43 (d, J=7.6 Hz, 1H), 5.24 (d, J=15.4 Hz, 1H), 4.74 (dd, J=9.6, 5.7 Hz, 1H), 4.39 (d, J=15.4 Hz, 1H), 3.92 (s, 3H), 3.64 (dd, J=13.9, 5.7 Hz, 1H), 2.81 (dd, J=13.9, 9.7 Hz, 1H), 2.29 (s, 3H).

### 3-((6-chloro-4-fluoro-2-((6-methoxypyridin-3-yl)methyl)-3-oxoisoindolin-1-yl)methyl)-4-methylpicolinonitrile (596)

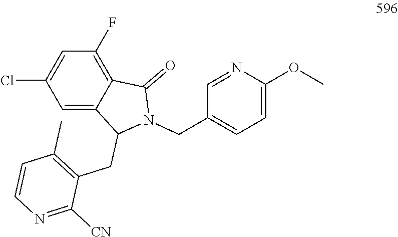 [SMILES: COc1ccc(CN2C(=O)c3c(F)cc(Cl)cc3C2Cc2c(C)ccnc2C#N)cn1]

The title compound 596 was prepared according to the procedure described for 588 as a white solid (5 mg, 7.2%). MS (ESI) m/z 437 [M+H]⁺. ¹H NMR (400 MHz, CDCl₃) δ 8.59 (d, J=4.9 Hz, 1H), 8.02 (d, J=2.4 Hz, 1H), 7.57 (dd, J=8.6, 2.5 Hz, 1H), 7.38 (d, J=4.9 Hz, 1H), 7.16 (dd, J=8.7, 1.5 Hz, 1H), 6.71 (d, J=8.6 Hz, 1H), 6.30 (s, 1H), 5.14 (d, J=15.3 Hz, 1H), 4.77 (dd, J=9.0, 6.3 Hz, 1H), 4.23 (d, J=15.3 Hz, 1H), 3.92 (s, 3H), 3.59 (dd, J=14.0, 6.3 Hz, 1H), 2.89 (dd, J=14.0, 9.1 Hz, 1H), 2.11 (s, 3H).

### 5-((2-(4-methoxybenzyl)-3-oxoisoindolin-1-yl)methyl)-6-methylpyrimidine-4-carbonitrile (597)

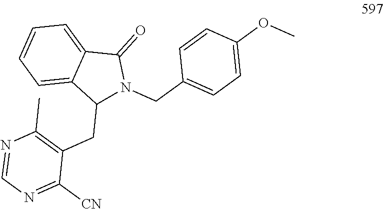 [SMILES: COc1ccc(CN2C(=O)c3ccccc3C2Cc2c(C)ncnc2C#N)cc1]

The title compound 597 was prepared according to the procedure described for 384 as a white solid (50 mg, 26%). MS (EST) m/z 385 [M+H]⁺. ¹H NMR (400 MHz, CDCl₃) δ 9.14 (s, 1H), 7.95 (d, J=7.6 Hz, 1H), 7.51 (t, J=7.5 Hz, 11H), 7.39 (td, J=7.5, 1.2 Hz, 1H), 7.23-7.15 (m, 2H), 6.90-6.82 (m, 2H), 6.45 (d, J=7.6 Hz, 1H), 5.30 (d, J=15.2 Hz, 1H), 4.72 (dd, J=9.3, 6.1 Hz, 1H), 4.30 (d, J=15.2 Hz, 1H), 3.79 (s, 3H), 3.60 (dd, J=13.9, 6.0 Hz, 1H), 2.82 (dd, J=13.9, 9.3 Hz, 1H), 2.26 (s, 3H).

### 3-((2-((6-methoxypyridin-3-yl)methyl)-3-oxoisoindolin-1-yl)methyl)pyridine-2,4-dicarbonitrile (598)

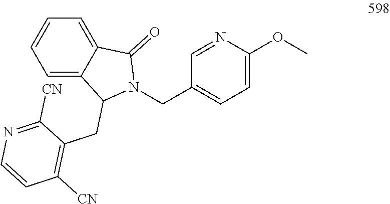 [SMILES: COc1ccc(CN2C(=O)c3ccccc3C2Cc2c(C#N)ccnc2C#N)cn1]

The title compound 598 was prepared according to the procedure described for 590 as a white solid (110 mg, 56%). MS (ESI) m/z 396 [M+H]+H NMR (400 MHz, CDCl₃) δ 8.83 (d, J=4.8 Hz, 1H), 8.07 (d, J=2.1 Hz, 1H), 7.90 (d, J=7.5 Hz, 1H), 7.74 (d, J=4.8 Hz, 1H), 7.62 (dd, J=8.5, 2.3 Hz, 1H), 7.54-7.39 (m, 2H), 6.71 (d, J=8.5 Hz, 1H), 6.64 (d, J=7.5 Hz, 1H), 5.22 (d, J=15.5 Hz, 1H), 4.87 (dd, J=8.0, 5.4 Hz, 1H), 4.48 (d, J=15.5 Hz, 1H), 3.92 (s, 3H), 3.82 (dd, J=13.9, 5.3 Hz, 1H), 3.14 (dd, J=13.9, 8.2 Hz, 1H).

### 3-((4-chloro-1-methyl-1H-pyrazol-5-yl)methyl)-2-((6-methoxyspiro[3.3]heptan-2-yl)methyl)isoindolin-1-one (599)

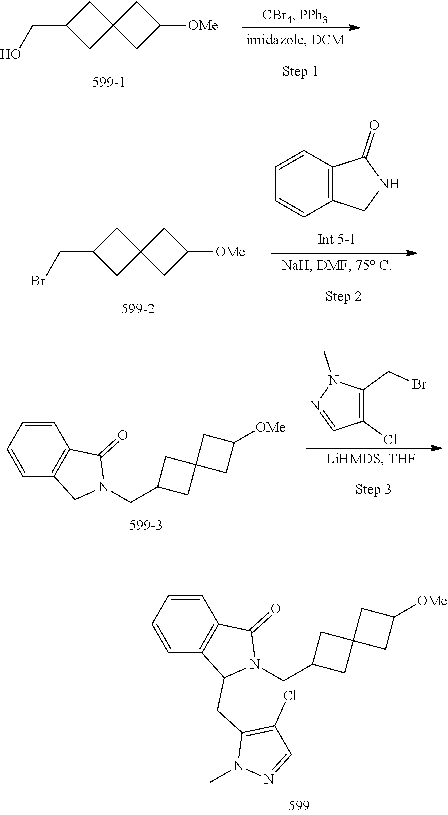

Step 1 was performed according to the procedure outlined for the preparation of 321-4.

Step 2 was performed according to the procedure outlined for the preparation of 270-2.

Step 3 was performed according to the procedure outlined for the preparation of Int 12-2 to afford 599 (70 mg, 24%) as a white solid. MS (ESI) m/z 400 [M+H]⁺. ¹H NMR (400 MHz, CDCl₃) δ 7.85 (dd, J=7.5, 2.4 Hz, 1H), 7.50 (d, J=2.4 Hz, 1H), 7.48-7.37 (m, 2H), 6.51 (d, J=7.6 Hz, 1H), 4.83-4.79 (m, 1H), 4.23-4.06 (m, 1H), 3.79-3.68 (m, 1H), 3.52-3.41 (m, 1H), 3.44 (s, 3H), 3.21-3.06 (m, 1H), 3.19 (s, 3H), 2.67-2.48 (m, 2H), 2.44-2.11 (m, 2H), 2.07-1.97 (m, 1H), 1.95-1.75 (m, 5H).

### 2-(benzo[c][1,2,5]oxadiazol-5-ylmethyl)-3-((4-chloro-1-methyl-IH-pyrazol-5-yl)methyl)isoindolin-1-one (600)

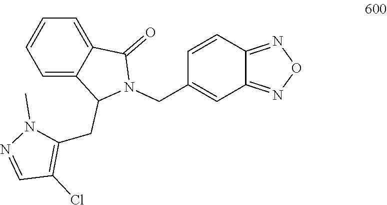 [SMILES: Cn1ncc(Cl)c1CC1c2ccccc2C(=O)N1Cc1ccc2nonc2c1]

The title compound 600 was prepared according to the procedure described for compound 502 as a white solid (40 mg, 38%). MS (ESI) m/z 394 [M+H]⁺. ¹H NMR (400 MHz, CDCl₃) δ 7.94 (d, J=7.0 Hz, 1H), 7.79 (d, J=9.3 Hz, 1H), 7.62 (s, 1H), 7.57-7.44 (m, 3H), 7.33 (dd, J=9.3, 1.4 Hz, 1H), 6.70 (d, J=7.4 Hz, 1H), 5.39 (dd, J=15.7, 1.3 Hz, 1H), 4.75 (dd, J=8.8, 6.2 Hz, 1H), 4.31 (d, J=15.7 Hz, 1H), 3.49 (s, 3H), 3.38 (dd, J=14.5, 6.2 Hz, 1H), 2.77 (dd, J=14.5, 8.8 Hz, 1H).

### (Z)-3-((4-chloro-1-methyl-1H-pyrazol-5-yl)methyl)-2-(2-fluoro-3-(5-fluoro-1H-1,2,3-triazol-4-yl)allyl)isoindolin-1-one (601)

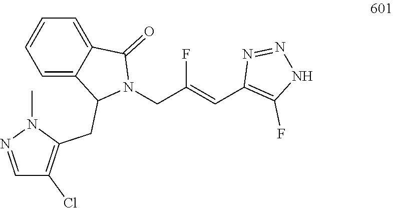 [SMILES: Cn1ncc(Cl)c1CC1c2ccccc2C(=O)N1C/C(F)=C/c1nn[nH]c1F]

The title compound 601 was prepared according to the procedure described for compound 516 as a white solid (6 mg, 26.5%). MS (ESI) m/z 405 [M+H]⁺. ¹H NMR (400 MHz, CD₃OD) δ 7.84-7.79 (m, 1H), 7.58-7.47 (m, 3H), 6.89-6.85 (m, 1H), 5.92 (d, J=38.4 Hz, 1H), 5.06-4.99 (m, 2H), 4.13 (dd, J=23.6, 16.0 Hz, 1H), 3.63 (s, 3H), 3.63 (dd, J=14.8, 5.9 Hz, 1H), 2.98 (dd, J=14.4, 8.8 Hz, 1H).

The intermediate 601-3 was prepared as follows:

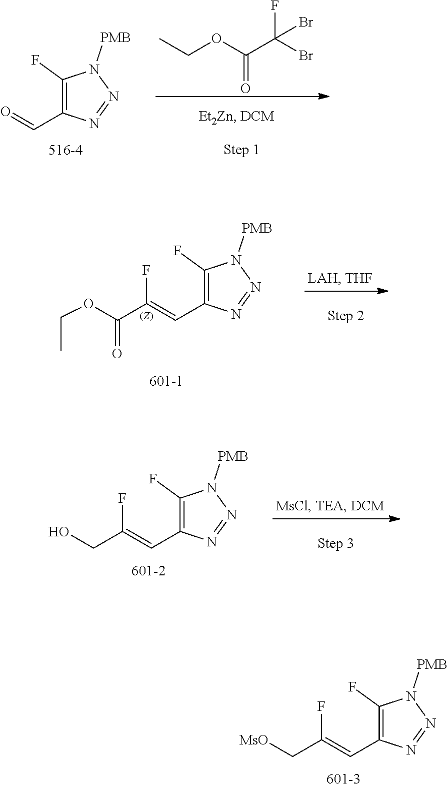

### Step 1. ethyl (Z)-2-fluoro-3-(5-fluoro-1-(4-methoxybenzyl)-1H-1,2,3-triazol-4-yl)acrylate (601-1)

To a solution of 516-4 (2 g, 8.5 mmol) and ethyl 2,2-dibromo-2-fluoroacetate (4.4 g, 17 mmol) in DCM (60 mL) was added ZnEt₂ (34 mL, 34 mmol, 1.0 M in hexane) at r.t. under N₂. The mixture was stirred at 40′C for 2 h. The mixture was cooled to r.t., added sat. NH₄Cl (50 mL) and extracted with DCM (50 mL*2). The combined organic layers were concentrated under vacuum. The residue was purified by FCC (PE/EA=2/1) to give 601-1 (300 mg, 10%) as a yellow oil. MS (ESI) m/z 324 [M+H]⁺.

### Step 2. (Z)-2-fluoro-3-(5-fluoro-1-(4-methoxybenzyl)-1H-1,2,3-triazol-4-yl)prop-2-en-1-ol (601-2)

To a solution of 601-1 (150 mg, 0.46 mmol) in THF (2 mL) was added LAH (0.5 mL, 0.56 mmol, 1M in THF) at −40′C. The mixture was stirred at −40° C. for 2 h. The mixture was quenched with water (5 mL), and extracted with EA (5 mL*3). The combined organic layers were concentrated under vacuum. The residue was purified by FCC (PE/EA=1/1) to give 601-2 (20 mg, 15%) as a yellow oil. MS (ESI) m/z 282 [M+H]⁺.

Step 3 was performed according to the procedure outlined for the preparation of 132-2 to afford 601-3 as a yellow oil. MS (ESI) m/z 360 [M+H]⁺.

### 3-((4-chloro-1-methyl-1H-pyrazol-5-yl)methyl)-2-((2-methoxyoxazol-5-yl)methyl)isoindolin-1-one (603) and 3-((4-chloro-1-methyl-1H-pyrazol-5-yl)methyl)-2-((2-hydroxyoxazol-5-yl)methyl)isoindolin-1-one (605)

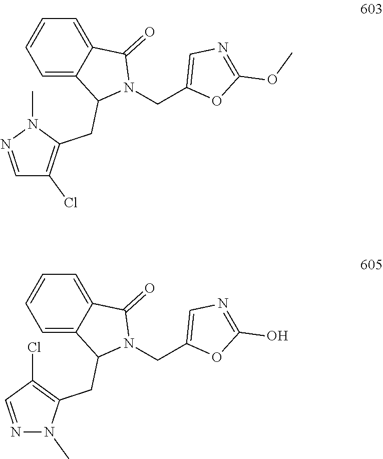 [SMILES: COc1ncc(CN2C(=O)c3ccccc3C2Cc2c(Cl)cnn2C)o1 ; Cn1ncc(Cl)c1CC1c2ccccc2C(=O)N1Cc1cnc(O)o1]

The title compound 603 was prepared according to the procedure described for compound 591 as a white solid (50 mg, 28%). MS (ESI) m/z 373 [M+H]⁺. ¹H NMR (400 MHz, CD₃OD) δ 7.81-7.79 (m, 11H), 7.52-7.51 (m, 3H), 6.88-6.81 (m, 1H), 6.81 (s, 1H), 5.22 (d, J=16.4 Hz, 1H), 4.89 (dd, J=8.4, 6.0 Hz, 1H), 4.37 (d, J=16.4 Hz, 1H), 4.01 (s, 3H), 3.63 (s, 3H), 3.58 (dd, J=15.2, 6.0 Hz, 1H), 2.97 (dd, J=14.8, 8.8 Hz, 1H).

The title compound 605 was prepared according to the procedure described for compound 602 as a white solid (14.8 mg, 39%). MS (ESI) m/z 359 [M+H]⁺. 11H NMR (400 MHz, CD₃OD) δ 7.81-7.79 (m, 1H), 7.55-7.50 (m, 3H), 6.87-6.82 (m, 1H), 6.78 (s, 1H), 5.07 (d, J=16.4 Hz, 1H), 4.96 (dd, J=8.8, 6.0 Hz, 1H), 4.24 (d, J=16.0 Hz, 1H), 3.65 (s, 3H), 3.60 (dd, J=14.8, 5.6 Hz, 1H), 2.97 (dd, J=14.8, 8.8 Hz, 1H).

### (E)-2-(3-(1H-1,2,3-triazol-4-yl)allyl)-3-((4-chloro-1-methyl-1H-pyrazol-5-yl)methyl)-1,1-dimethylisoindoline (606)

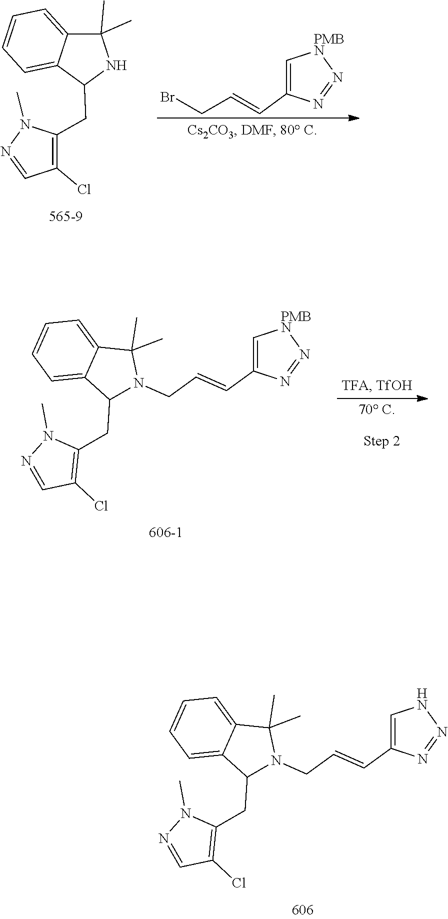

### Step 1. (E)-3-((4-chloro-1-methyl-1H-pyrazol-5-yl)methyl)-2-(3-(1-(4-methoxybenzyl)-1H-1,2,3-triazol-4-yl)allyl)-1,1-dimethylisoindoline (606-1)

To a solution of 565-9 (200 mg, 0.72 mmol) and Cs₂CO₃ (473 mg, 1.45 mmol) in DMF (2 mL) was added (E)-4-(3-bromoprop-1-en-1-yl)-1-(4-methoxybenzyl)-lH-1,2,3-triazole (223 mg, 0.72 mmol) under N₂. The mixture was stirred at 80° C. for 2 h, then diluted with EA, and washed with water. The organic layer was separated, dried over anhydrous Na₂SO₄ and concentrated under vacuum. The residue was purified by prep TLC (PE/EA=1/2) to give 606-1 (190 mg, 52%) as a yellow solid. MS (ESI) m/z 503 [M+H]⁺.

Step 2 was performed according to the procedure outlined for step 2 of 4 to afford 606 (29 mg, 19%) as a white solid. MS (ESI) m/z 383 [M+H]⁺. ¹H NMR (400 MHz, DMSO-d₆) δ 14.78 (s, 1H), 7.94 (s, 1H), 7.48 (s, 1H), 7.29-7.20 (m, 2H), 7.17-7.07 (m, 1H), 6.63-6.54 (m, 2H), 6.49-6.37 (m, 1H), 4.49 (dd, J=7.8, 5.8 Hz, 1H), 3.63 (s, 3H), 3.61-3.51 (m, 2H), 3.20 (dd, J=14.4, 5.6 Hz, 1H), 2.88 (dd, J=14.5, 7.9 Hz, 1H), 1.42 (s, 3H), 1.28 (s, 3H).

### 3-((3-((4-chloro-1-methyl-1H-pyrazol-5-yl)methyl)-1,1-dimethylisoindolin-2-yl)methyl)cyclobutane-1-carboxamide (607)

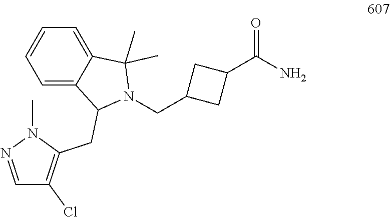 [SMILES: Cn1ncc(Cl)c1CC1c2ccccc2C(C)(C)N1CC1CC(C(N)=O)C1]

The title compound 607 was prepared according to the procedure described for compound 404 as a white solid (17 mg, 10.3%). MS (ESI) m/z 387 [M+H]⁺. ¹H NMR (400 MHz, DMSO-d₆) δ 7.55-7.49 (m, 1H), 7.28-7.18 (m, 2H), 7.10 (s, 1H), 7.07-7.00 (m, 1H), 6.65 (s, 1H), 6.26-6.19 (m, 1H), 4.45-4.34 (m, 1H), 3.50 (s, 2H), 3.49 (s, 1H), 3.29-3.20 (m, 1H), 3.05-2.95 (m, 1H), 2.95-2.78 (m, 1H), 2.76-2.61 (m, 2H), 2.48-2.43 (m, 1H), 2.32-2.08 (m, 2H), 1.88-1.70 (m, 2H), 1.38 (s, 1H), 1.37 (s, 2H), 1.28 (s, 1H), 1.25 (s, 2H).

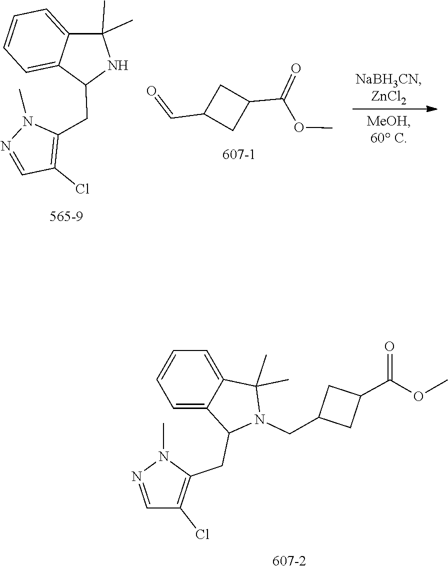

The intermediate 607-2 was prepared according to the procedure described for 565-10.

### 5-((3-((4-chloro-1-methyl-IH-pyrazol-5-yl)methyl)-1,1-dimethylisoindolin-2-yl)methyl)-1H-benzo[d][1,2,3]triazole (608)

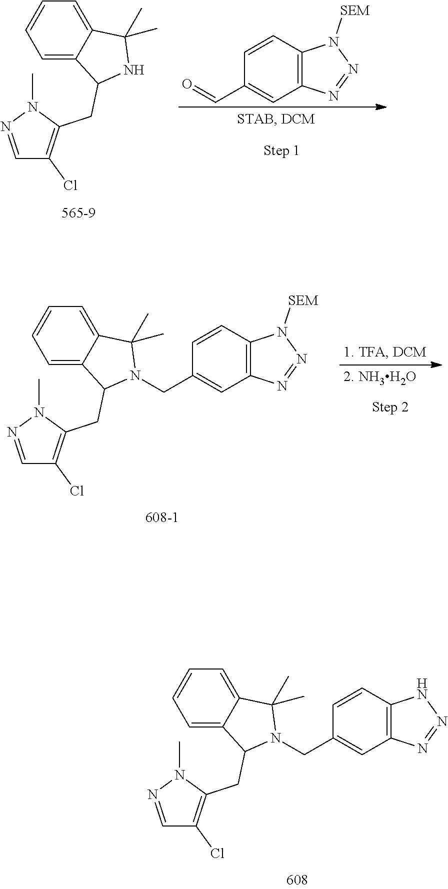 [SMILES: Cn1ncc(Cl)c1CC1NC(C)(C)c2ccccc21 ; C[Si](C)(C)CCOCn1nnc2cc(C=O)ccc21 ; Cn1ncc(Cl)c1CC1c2ccccc2C(C)(C)N1Cc1ccc2c(c1)nnn2COCC[Si](C)(C)C ; Cn1ncc(Cl)c1CC1c2ccccc2C(C)(C)N1Cc1ccc2[nH]nnc2c1]

### Step 1. 5-((3-((4-chloro-1-methyl-1H-pyrazol-5-yl)methyl)-1,1-dimethylisoindolin-2-yl)methyl)-1-((2-(trimethylsilyl)ethoxy)methyl)-1H-benzo[d][1,2,3]triazole (608-1)

To a solution of 565-9 (200 mg, 0.72 mmol) in DCM (5 mL) was added 1-((2-(trimethylsilyl)ethoxy)methyl)-1H-benzo[d][1,2,3]triazole-5-carbaldehyde (221 mg, 0.79 mmol) and STAB (461 mg, 2.18 mmol) under N₂. The mixture was stirred for 14 h, then diluted with DCM, and washed with water. The organic layer was separated, dried over anhydrous Na₂SO₄ and concentrated under vacuum. The residue was purified by silica gel column 20 chromatography (PE/EA=3/1) to give 608-1 (160 mg, 41%) as a yellow solid. MS (ESI) m/z 537 [M+H]⁺.

Step 2 was performed according to the procedure outlined for step 2 of preparation of 146 to give 608 (51 mg, 40.4%) as a white solid. MS (ESI) m/z 407 [M+H]⁺. ¹H NMR (400 MHz, DMSO-d₆) δ 15.46 (s, 1H), 7.92 (s, 1H), 7.87 (d, J=8.6 Hz, 1H), 7.54 (dd, J=8.5, 1.4 Hz, 1H), 7.44 (s, 1H), 7.32-7.22 (m, 2H), 7.12-7.03 (m, 1H), 6.25 (d, J=7.5 Hz, 1H), 4.41 (dd, J=9.8, 4.7 Hz, 1H), 4.27 (d, J=14.8 Hz, 1H), 4.18 (d, J=14.7 Hz, 1H), 3.37 (s, 3H), 3.11 (dd, J=14.4, 4.8 Hz, 1H), 2.67 (dd, J=14.5, 9.7 Hz, 1H), 1.40 (s, 3H), 1.39 (s, 3H).

### 3-((3-chloro-1-methyl-6-oxo-1,6-dihydropyridin-2-yl)methyl)-2-((6-methoxypyridin-3-yl)methyl)isoindolin-1-one (609)

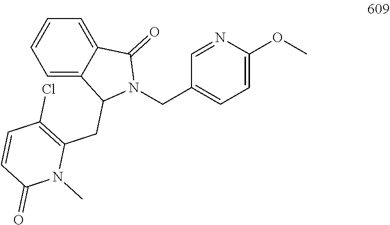 [SMILES: COc1ccc(CN2C(=O)c3ccccc3C2Cc2c(Cl)ccc(=O)n2C)cn1]

The title compound 609 was prepared according to the procedure described for 588 using 381-8 as a starting material to afford a white solid (76 mg, 26%). MS (ESI) m/z 410 [M+H]⁺. ¹H NMR (400 MHz, DMSO-d₆) δ 8.10 (d, J=2.2 Hz, 1H), 7.82-7.73 (m, 1H), 7.58 (dd, J=8.5, 2.5 Hz, 1H), 7.54-7.47 (m, 3H), 6.85-6.81 (m, 1H), 6.79 (d, J=8.5 Hz, 1H), 6.49 (d, J=9.7 Hz, 1H), 5.12 (d, J=15.3 Hz, 1H), 4.82 (dd, J=9.0, 6.7 Hz, 1H), 4.30 (d, J=15.4 Hz, 1H), 3.82 (s, 3H), 3.70 (dd, J=14.4, 6.7 Hz, 1H), 3.27 (s, 3H), 3.01 (dd, J=14.4, 9.2 Hz, 1H).

### 2-((1-((4-chloro-1-methyl-1H-pyrazol-5-yl)methyl)-3,4-dihydroisoquinolin-2(1H)-yl)methyl)-5-oxa-7-azaspiro[3.4]octan-6-one (610)

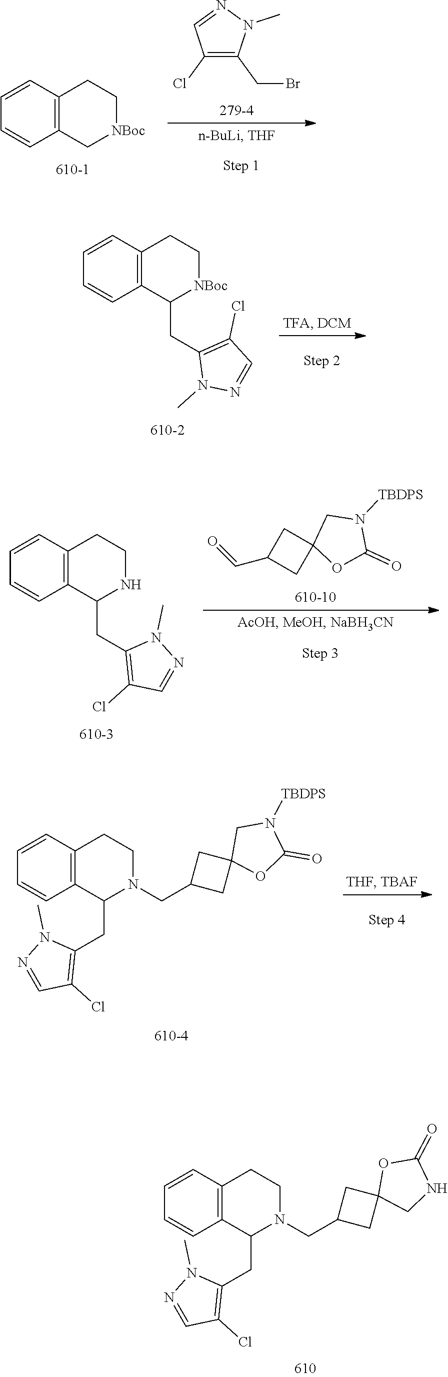

### Step 1: tert-butyl 1-((4-chloro-1-methyl-1H-pyrazol-5-yl)methyl)-3,4-dihydroisoquinoline-2(1H)-carboxylate (610-2)

To a solution of tert-butyl 3,4-dihydroisoquinoline-2(1H)-carboxylate (500 mg, 2.15 mmol) in dried THF (15 mL) was added n-Buli (2.4 M, 4.3 mmol, 1.8 mL) dropwise at −50° C. under N₂. The mixture was stirred for 20 min, and then 279-4 (900 mg, 4.3 mmol) was added. The mixture was stirred for 30 min at −50° C., then quenched with NH₄Cl (aq.) (5 mL) and extracted with Ethyl acetate (2*20 mL). The combined organic layer was dried over anhydrous Na₂SO₄ and concentrated. The residue was purified by column chromatography on silica gel (eluting PE/EA=1/2 to 1/1) to give 610-2 (190 mg, 50% of purity) as a yellow oil, which was used for next step without further purification. MS (ESI) m/z 362 [M+H]⁺.

Step 2 was performed according to the procedure outlined for step 3 of preparation of Int 12.

### Step 3: 7-(tert-butyldiphenylsilyl)-2-((1-((4-chloro-1-methyl-1H-pyrazol-5-yl)methyl)-3,4-dihydroisoquinolin-2(1H)-yl)methyl)-5-oxa-7-azaspiro[3.4]octan-6-one (610-4)

To a solution of 610-3 (20 mg, 0.07 mmol) and 610-10 (55 mg, 0.14 mmol) in MeOH (5 mL) was added AcOH (1 drop) and NaBH₃CN (8 mg, 0.14 mmol). The mixture was stirred for 2 h at r.t., and then diluted with water (5 mL). The organic layer was washed with water (10 mL) and extracted with EA (10 mL), dried over Na₂SO₄, filtered, and concentrated in vacuo. The residue was purified by column chromatography on silica gel (eluting PE/EA=3/1 to 2/1) to give 610-4 (20 mg, 45%) as a yellow oil. MS (ESI) m/z 639 [M+H]⁺.

Step 4 was performed according to the procedure outlined for step 5 of preparation of 321 to give 610 (2 mg, 6%) as a white solid. MS (ESI) m/z 401 [M+H]⁺. ¹H NMR (400 MHz, CDCl₃) δ 7.40 (s, 1H), 7.22-7.09 (m, 3H), 6.90 (s, 1H), 4.98 (s, 1H), 3.87 (s, 1H), 3.61 (s, 3H), 3.56 (s, 2H), 3.46-3.36 (m, 1H), 3.05-2.95 (m, 3H), 2.91 (dd, J=14.5, 5.3 Hz, 1H), 2.76-2.57 (m, 3H), 2.27-2.18 (m, 2H), 2.08-1.97 (m, 3H).

The intermediate 610-10 was prepared as follows:

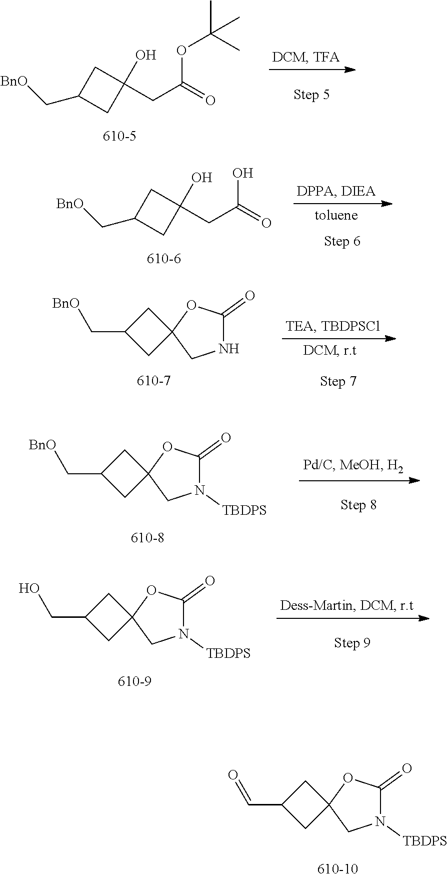

610-5 was prepared according to the procedure outlined for preparation of 270-4.

Step 5 was performed according to the procedure outlined for step 3 of preparation of 270-5.

Step 6 was performed according to the procedure outlined for step 5 of preparation of 270.

Step 7 was performed according to the procedure outlined for preparation of 445-1.

Step 8 was performed according to the procedure outlined for step 1 of preparation of 1.

Step 9 was performed according to the procedure outlined for preparation of 361-4.

### 3-((4-chloro-1-methyl-1H-pyrazol-5-yl)methyl)-2-((6-oxo-5,7-diazaspiro[3.4]octan-2-yl)methyl)isoindolin-1-one (611)

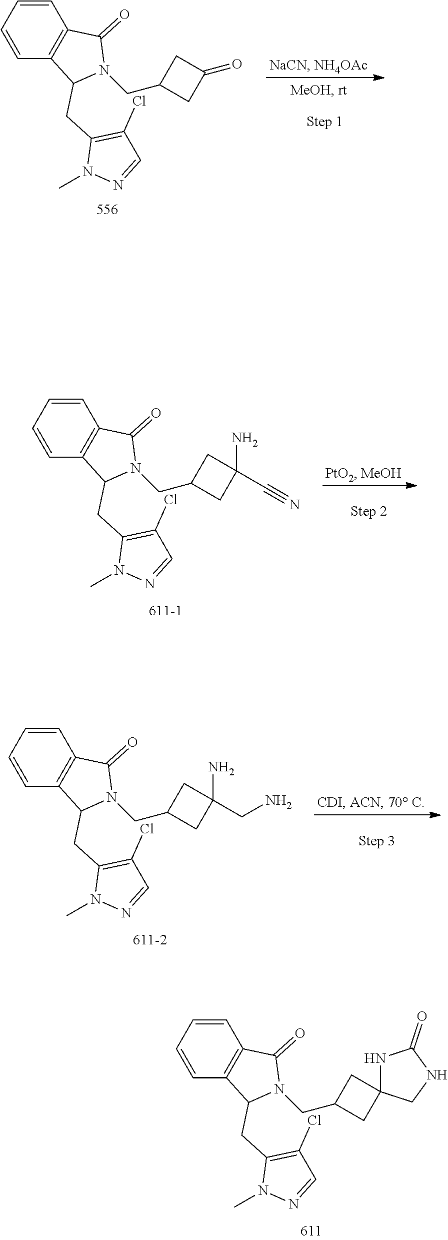 [SMILES: Cn1ncc(Cl)c1CC1c2ccccc2C(=O)N1CC1CC(=O)C1 ; Cn1ncc(Cl)c1CC1c2ccccc2C(=O)N1CC1CC(N)(C#N)C1 ; Cn1ncc(Cl)c1CC1c2ccccc2C(=O)N1CC1CC(N)(CN)C1 ; Cn1ncc(Cl)c1CC1c2ccccc2C(=O)N1CC1CC2(CNC(=O)N2)C1]

### Step 1. 1-amino-3-((1-((4-chloro-1-methyl-1H-pyrazol-5-yl)methyl)-3-oxoisoindolin-2-yl)methyl)cyclobutane-1-carbonitrile (611-1)

To a solution of 556 (500 mg, 1.46 mmol) in MeOH (20 mL) was added NaCN (86 mg, 1.75 mmol) and NH₄0Ac (898 mg, 11.66 mmol) at r.t. The mixture was stirred at r.t. overnight. The mixture was poured into water (20 mL), and extracted with DCM (20 mL*3), and then the combined organic layers were concentrated and purified by FCC (DCM/MeOH=20/1) to give 611-1 (200 mg, 37%) as a white solid. MS (ESI) m/z 370 [M+H]⁺.

### Step 2. 2-((3-amino-3-(aminomethyl)cyclobutyl)methyl)-3-((4-chloro-1-methyl-1H-pyrazol-5-yl)methyl)isoindolin-1-one (611-2)

To a solution of 611-1 (190 mg, 0.51 mmol) in MeOH (10 mL) was added PtO₂ (50 mg) at r.t. The mixture was stirred at 30 TC overnight under 1 atm H₂. The mixture was filtered and the filtrate was concentrated to give 611-2 (150 mg, 78%) as a yellow solid. MS (ESI) m/z 374 [M+H]⁺.

### Step 3. 3-((4-chloro-1-methyl-IH-pyrazol-5-yl)methyl)-2-((6-oxo-5,7-diazaspiro[3.4]octan-2-yl)methyl)isoindolin-1-one (611)

A mixture of 611-2 (50 mg, 0.13 mmol) and CDI (33 mg, 0.20 mmol) in ACN (5 mL) was stirred at 70° C.T for 1 h. The mixture was diluted with DCM (10 mL), and washed with H₂O (10 mL*2). The organic layer was dried over Na₂SO₄, filtered, concentrated and purified by prep-HPLC to give 611 (9 mg, 16.6%) as a white solid. MS (ESI) m/z 400 [M+H]⁺. ¹H NMR (400 MHz, CD₃OD) δ 7.78-7.76 (m, 1H), 7.52-7.49 (m, 3H), 6.77-6.75 (m, 1H), 4.99 (dd, J=8.8, 5.6 Hz, 1H), 4.09 (dd, J=14.0, 7.2 Hz, 1H), 3.61-3.52 (m, 3H), 3.57 (s, 3H), 3.35 (dd, J=14.2, 6.0 Hz, 1H), 2.87 (dd, J=14.8, 9.2 Hz, 1H), 2.39-2.35 (m, 2H), 2.25-2.02 (m, 3H).

### 6-((3-((4-chloro-1-methyl-1H-pyrazol-5-yl)methyl)-1,1-dimethylisoindolin-2-yl)methyl)benzo[d]oxazol-2(3H)-one (613)

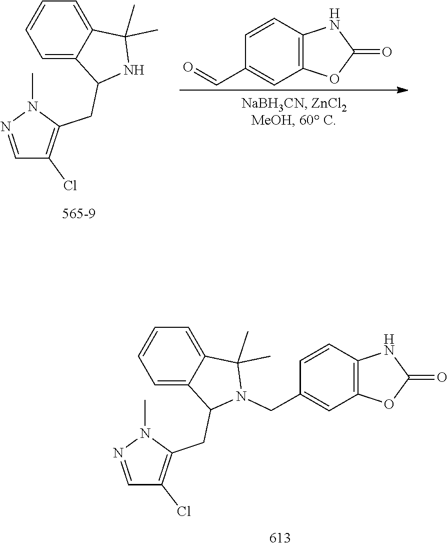 [SMILES: Cn1ncc(Cl)c1CC1NC(C)(C)c2ccccc21 ; O=Cc1ccc2[nH]c(=O)oc2c1 ; Cn1ncc(Cl)c1CC1c2ccccc2C(C)(C)N1Cc1ccc2[nH]c(=O)oc2c1]

The title compound 613 was prepared according to the procedure described for 565-10 as a white solid (36 mg, 11%). MS (ESI) m/z 423 [M+H]⁺. ¹H NMR (400 MHz, DMSO-d₆) δ 11.62 (s, 1H), 7.46 (s, 1H), 7.32 (d, J=1.4 Hz, 1H), 7.31-7.20 (m, 3H), 7.13-6.98 (m, 2H), 6.28 (d, J=7.4 Hz, 1H), 4.38 (dd, J=9.7, 4.8 Hz, 1H), 4.10 (d, J=14.6 Hz, 1H), 4.01 (d, J=14.6 Hz, 1H), 3.42 (s, 3H), 3.09 (dd, J=14.5, 4.9 Hz, 1H), 2.66 (dd, J=14.4, 9.8 Hz, 1H), 1.38 (s, 3H), 1.34 (s, 3H).

### 5-((3-((4-chloro-1-methyl-1H-pyrazol-5-yl)methyl)-1,1-dimethylisoindolin-2-yl)methyl)thiazol-2-ol (614)

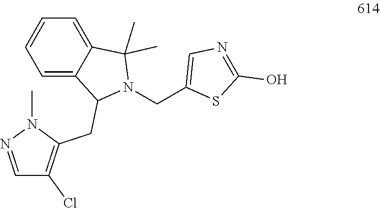 [SMILES: Cn1ncc(Cl)c1CC1c2ccccc2C(C)(C)N1Cc1cnc(O)s1]

The title compound 614 was prepared according to the procedure described for compound 592 as a white solid (15 mg, 13%). MS (ESI) m/z 389 [M+H]⁺. ¹H NMR (400 MHz, DMSO-d₆) δ 10.88 (s, 1H), 7.51 (s, 1H), 7.29-7.20 (m, 2H), 7.12-7.03 (m, 1H), 6.87 (d, J=2.5 Hz, 1H), 6.32 (d, J=7.7 Hz, 1H), 4.46 (dd, J=9.4, 5.0 Hz, 1H), 4.00 (d, J=15.0 Hz, 1H), 3.86-3.78 (m, 1H), 3.51 (s, 3H), 3.29 (dd, J=14.3, 5.0 Hz, 1H), 2.71 (dd, J=14.3, 9.5 Hz, 1H), 1.40 (s, 3H), 1.30 (s, 3H).

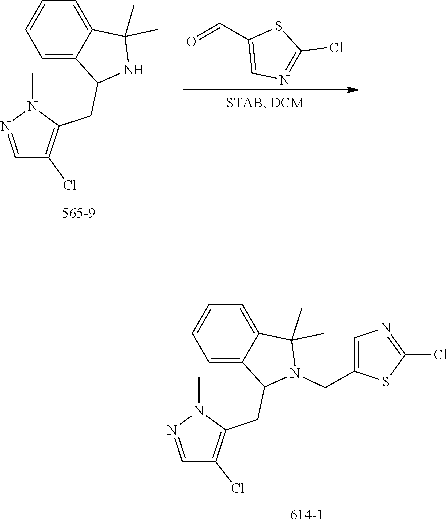

614-1 was prepared according to the procedure outlined for the preparation of 608-1.

### 6-((4-chloro-1-methyl-1H-pyrazol-5-yl)methyl)-5-(4-methoxybenzyl)-5,6-dihydro-4H-pyrrolo[3,4-d]thiazol-4-one (615)

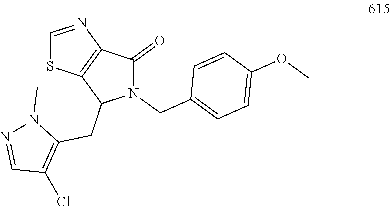 [SMILES: COc1ccc(CN2C(=O)c3ncsc3C2Cc2c(Cl)cnn2C)cc1]

The title compound 615 was the intermediate 430-6. It was prepared according to the procedure described for intermediate 430-6 as a white solid. MS (ESI) m/z 389 [M+H]⁺. ¹H NMR (400 MHz, DMSO-d₆) δ 9.18 (s, 1H), 7.56 (s, 1H), 7.28-7.20 (m, 2H), 6.96-6.88 (m, 2H), 5.08 (d, J=15.2 Hz, 1H), 4.82 (dd, J=9.5, 5.8 Hz, 1H), 4.31 (d, J=15.2 Hz, 1H), 3.73 (s, 3H), 3.66 (s, 3H), 3.61 (dd, J=14.6, 5.9 Hz, 1H), 2.90 (dd, J=14.6, 9.5 Hz, 1H).

### 2-((3-((4-chloro-1-methyl-1H-pyrazol-5-yl)methyl-d₂)-1,1-dimethylisoindolin-2-yl)methyl)-5-oxa-7-azaspiro[3.4]octan-6-one (616)

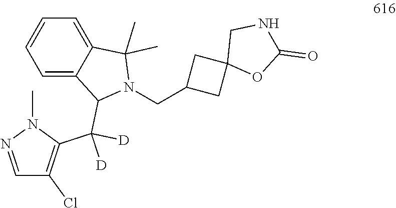 [SMILES: [2H]C([2H])(c1c(Cl)cnn1C)C1c2ccccc2C(C)(C)N1CC1CC2(CNC(=O)O2)C1]

The title compound 616 was prepared according to the procedure described for compound 565 as a white solid (12 mg, 15%). MS (ESI) m/z 417 [M+H]⁺. ¹H NMR (400 MHz, DMSO-d₆) δ 7.52 (s, 1H), 7.47 (s, 1H), 7.28-7.19 (m, 2H), 7.11-7.01 (m, 1H), 6.29 (d, J=7.5 Hz, 1H), 4.40 (s, 1H), 3.62-3.42 (m, 5H), 3.09-3.00 (m, 1H), 2.78-2.68 (m, 1H), 2.44-2.26 (m, 2H), 2.24-2.15 (m, 1H), 2.09-1.95 (m, 2H), 1.38 (s, 3H), 1.27 (s, 3H).

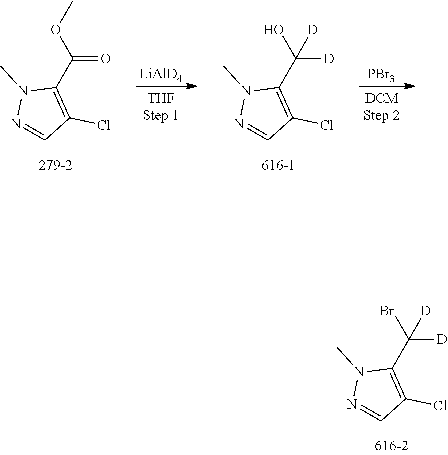

616-2 was prepared according to the procedure outlined for preparation of 279-4.

### (R)-3-((4-chloro-1-methyl-IH-pyrazol-5-yl)methyl)-2-(4-methoxybenzyl)isoindolin-1-one (617) and (S)-3-((4-chloro-1-methyl-1H-pyrazol-5-yl)methyl)-2-(4-methoxybenzyl)isoindolin-1-one (618)

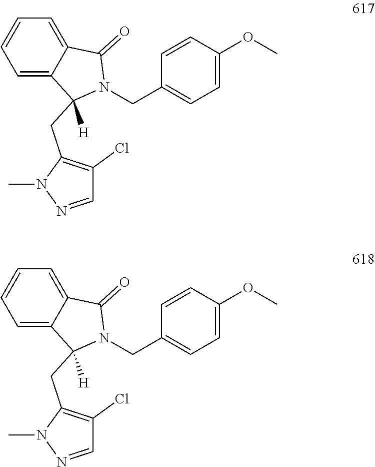 [SMILES: COc1ccc(CN2C(=O)c3ccccc3[C@H]2Cc2c(Cl)cnn2C)cc1 ; COc1ccc(CN2C(=O)c3ccccc3[C@@H]2Cc2c(Cl)cnn2C)cc1]

Chiral separation of compound 518 by chiral prep-HPLC (column: CHIRALPAK IA; column size: 5 cm×25 cm, 5 μm; mobile phase A: MTBE; mobile phase B: MeOH:DCM) was conducted to afford two enantiomers: compound 617 and compound 618. One of the two enantiomers was a white solid, with RT=2.253 min (CHIRAL HPLC, column: CHIRALPAK IA-3; column size: 4.6*50 mm, 3 μm; mobile phase: MTBE(0.1% DEA):(MeOH:DCM=1:1)=90:10; flow: 1 mL/min). MS (ESI) m/z 382 [M+H]⁺. ¹H NMR (400 MHz, CDCl₃) δ 7.91 (d, J=7.4 Hz, 1H), 7.50-7.38 (m, 3H), 7.21-7.15 (m, 2H), 6.89-6.81 (m, 2H), 6.60 (dd, J=7.5, 1.0 Hz, 1H), 5.36 (d, J=15.0 Hz, 1H), 4.65 (dd, J=9.2, 6.0 Hz, 1H), 4.14 (d, J=15.0 Hz, 1H), 3.78 (s, 3H), 3.44 (s, 3H), 3.39 (dd, J=14.5, 6.1 Hz, 1H), 2.65 (dd, J=14.5, 9.2 Hz, 1H). The other one of the two enantiomers was a white solid, with RT=2.932 min (CHIRAL HPLC, column: CHIRALPAK IA-3; column size: 4.6*50 mm, 3 μm; mobile phase: MTBE (0.1% DEA): (MeOH:DCM=1:1)=90:10; flow: 1 mL/min). MS (ESI) m/z 382 [M+H]⁺. ¹H NMR (400 MHz, CDCl₃) δ 7.91 (d, J=7.4 Hz, 1H), 7.52-7.37 (m, 3H), 7.21-7.11 (m, 2H), 6.90-6.78 (m, 2H), 6.60 (dd, J=7.5, 1.0 Hz, 1H), 5.36 (d, J=15.1 Hz, 1H), 4.64 (dd, J=9.2, 6.0 Hz, 1H), 4.14 (d, J=15.0 Hz, 1H), 3.78 (s, 3H), 3.44 (s, 3H), 3.39 (dd, J=14.5, 6.0 Hz, 1H), 2.65 (dd, J=14.5, 9.3 Hz, 1H).

### 3-((4-chloro-1-methyl-1H-pyrazol-5-yl)methyl)-5-(difluoromethoxy)-2-((6-methoxypyridin-3-yl)methyl)isoindolin-1-one (619)

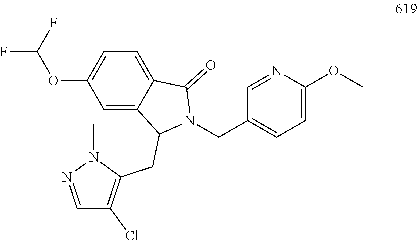 [SMILES: COc1ccc(CN2C(=O)c3ccc(OC(F)F)cc3C2Cc2c(Cl)cnn2C)cn1]

The title compound 619 was prepared according to the procedure described for 588 using 421-2 as a starting material to afford a white solid (86 mg, 52%). MS (EST) m/z 449 [M+H]⁺. ¹H NMR (400 MHz, CDCl₃) δ 8.08 (d, J=2.2 Hz, 1H), 7.89 (d, J=8.3 Hz, 1H), 7.54 (dd, J=8.6, 2.4 Hz, IH), 7.50 (s, 1H), 7.24 (dd, J=8.3, 2.1 Hz, 1H), 6.73 (d, J=8.6 Hz, 1H), 6.47 (t, J=32.8 Hz, 1H), 6.36 (d, J=2.0 Hz, 1H), 5.27 (d, J=15.2 Hz, 1H), 4.65 (dd, J=9.0, 6.0 Hz, 1H), 4.14 (d, J=15.3 Hz, 1H), 3.94 (s, 3H), 3.53 (s, 3H), 3.40 (dd, J=14.6, 6.0 Hz, 1H), 2.71 (dd, J=14.5, 9.2 Hz, 1H).

### 3-((4-chloro-1-methyl-1H-pyrazol-5-yl)methyl)-2-((6-methoxypyridin-3-yl)methyl)-3-methylisoindolin-1-one (620)

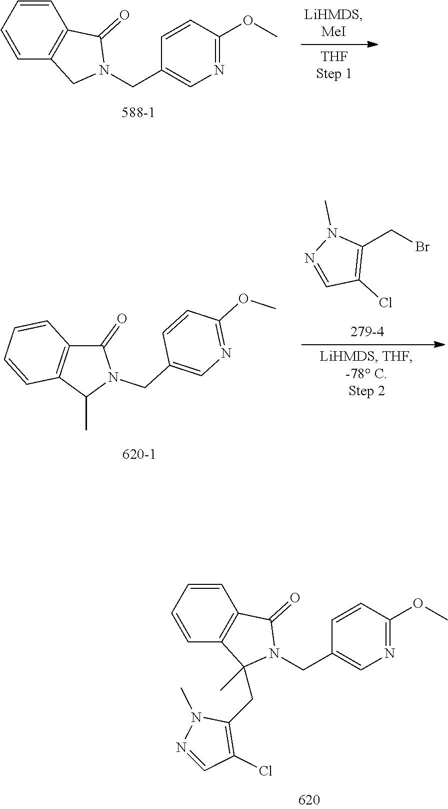

### Step 1: 2-((6-methoxypyridin-3-yl)methyl)-3-methylisoindolin-1-one (620-1)

To a solution of 588-1 (100 mg, 0.39 mmol) in dried THF (3 mL) was added LiHMDS (0.39 mL, 0.39 mmol) dropwise at −78° C. under N₂. The mixture was stirred for 40 min at −78° C., and then Mel (65.6 mg, 0.47 mmol) was added. The mixture was stirred for 1 h at −78° C., then quenched with NH₄Cl (aq.) (2 mL) and extracted with ethyl acetate (2*10 mL). The combined organic layer was dried over anhydrous Na₂SO₄ and concentrated. The residue was purified by column chromatography on silica gel (eluting PE/EA=1/2 to 1/1) to give 620-1 (50 mg, 47%) as a white solid. MS (ESI) m/z 269 [M+H]⁺.

Step 2 was performed according to the procedure outlined for 620-1 to give 620 (30 mg, 42%) 5 as a white solid. MS (ESI) m/z 397 [M+H]⁺. ¹H NMR (400 MHz, CDCl₃) δ 8.22 (d, J=2.1 Hz, 1H), 7.90 (d, J=7.3 Hz, 1H), 7.79 (dd, J=8.6, 2.3 Hz, 1H), 7.51-7.40 (m, 3H), 6.73 (d, J=8.6 Hz, 1H), 6.64 (d, J=7.5 Hz, 1H), 4.91 (d, J=15.6 Hz, 1H), 4.64 (d, J=15.6 Hz, 1H), 3.94 (s, 3H), 3.35 (d, J=14.5 Hz, 1H), 2.99 (s, 3H), 2.53 (d, J=14.5 Hz, 1H), 1.56 (s, 3H).

### 3-((2-(4-methoxybenzyl)-3-oxo-2,3-dihydro-1H-pyrrolo[3,4-c]pyridin-1-yl)methyl)-4-methylpicolinonitrile (621)

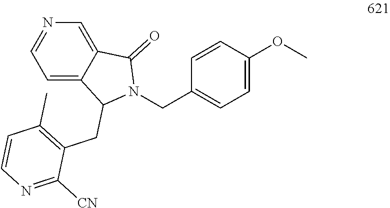 [SMILES: COc1ccc(CN2C(=O)c3cnccc3C2Cc2c(C)ccnc2C#N)cc1]

The title compound 621 was prepared according to the procedure described for compound 567 as a white solid (40 mg, 20%). MS (ESI) m/z 385 [M+H]⁺. ¹H NMR (400 MHz, CDCl₃) δ 9.18 (d, J=1.1 Hz, 1H), 8.59 (dd, J=9.8, 5.0 Hz, 2H), 7.36 (d, J=4.9 Hz, 1H), 7.20-7.14 (m, 2H), 6.89-6.81 (m, 2H), 6.47 (d, J=5.0 Hz, 1H), 5.27 (d, J=15.0 Hz, 1H), 4.81 (dd, J=9.2, 6.6 Hz, 1H), 4.25 (d, J=15.1 Hz, 1H), 3.78 (s, 3H), 3.61 (dd, J=14.0, 6.6 Hz, 1H), 2.91 (dd, J=14.0, 9.2 Hz, 1H), 2.06 (s, 3H).

### 5-bromo-3-((4-chloro-1-methyl-1H-pyrazol-5-yl)methyl)-2-((6-methoxypyridin-3-yl)methyl)isoindolin-1-one (622) and 3-((4-chloro-1-methyl-1H-pyrazol-5-yl)methyl)-5-cyclopropyl-2-((6-methoxypyridin-3-yl)methyl)isoindolin-1-one (623)

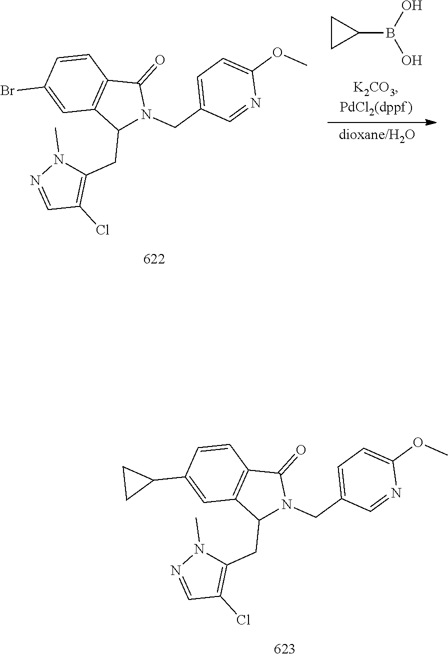

The title compound 622 was prepared according to the procedure described for compound 502 as a white solid (150 mg, 67%). MS (ESI) m/z 461 [M+H]⁺. ¹H NMR (400 MHz, DMSO-d₆) δ 8.03 (d, J=2.1 Hz, 1H), 7.72-7.62 (m, 2H), 7.55 (s, 1H), 7.53 (dd, J=8.6, 2.4 Hz, 1H), 7.16 (s, 1H), 6.78 (d, J=8.5 Hz, 1H), 5.10 (d, J=15.3 Hz, 1H), 4.71 (t, J=6.8 Hz, 1H), 4.27 (d, J=15.3 Hz, 1H), 3.82 (s, 3H), 3.65 (s, 3H), 3.49 (dd, J=14.9, 6.0 Hz, 1H), 3.07 (dd, J=14.9, 7.7 Hz, 1H).

The title compound 623 was prepared according to the procedure described for 26-1 as a white solid (12 mg, 20%). MS (ESI) m/z 423 [M+H]⁺. 1H NMR (400 MHz, DMSO-d₆) δ 8.05 (d, J=2.2 Hz, 1H), 7.59-7.51 (m, 3H), 7.28 (d, J=7.9 Hz, 1H), 6.78 (d, J=8.5 Hz, 1H), 6.35 (s, 1H), 5.09 (d, J=15.2 Hz, 1H), 4.58 (dd, J=8.0, 6.2 Hz, 1H), 4.25 (d, J=15.3 Hz, 1H), 3.82 (s, 3H), 3.58 (s, 3H), 3.49 (dd, J=14.8, 6.0 Hz, 1H), 2.92 (dd, J=14.8, 8.4 Hz, 1H), 1.98-1.88 (m, 1H), 1.03-0.91 (m, 2H), 0.63-0.55 (m, 2H).

### 3-((4-chloro-1-methyl-1H-pyrazol-5-yl)methyl)-5-cyclopropyl-2-(4-methoxybenzyl)isoindolin-1-one (624)

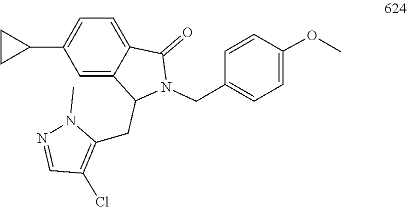 [SMILES: COc1ccc(CN2C(=O)c3ccc(C4CC4)cc3C2Cc2c(Cl)cnn2C)cc1]

The title compound 624 was prepared according to the procedure described for compound 623 as a white solid (15 mg, 26%). MS (ESI) m/z 422 [M+H]⁺. ¹H NMR (400 MHz, CDCl₃) δ 7.76 (d, J=7.9 Hz, 1H), 7.48 (s, 1H), 7.23-7.14 (m, 3H), 6.88-6.80 (m, 2H), 6.14 (s, 1H), 5.33 (d, J=15.0 Hz, 1H), 4.55 (dd, J=9.2, 6.2 Hz, 1H), 4.11 (d, J=15.0 Hz, 1H), 3.78 (s, 3H), 3.42 (s, 3H), 3.37 (dd, J=14.4, 6.1 Hz, 1H), 2.61 (dd, J=14.4, 9.3 Hz, 1H), 1.95-1.78 (m, 1H), 1.03-0.91 (m, 2H), 0.67-0.55 (m, 2H).

### 3-((4-chloro-1-methyl-2-oxo-1,2-dihydropyridin-3-yl)methyl)-2-((6-methoxypyridin-3-yl)methyl)isoindolin-1-one (625)

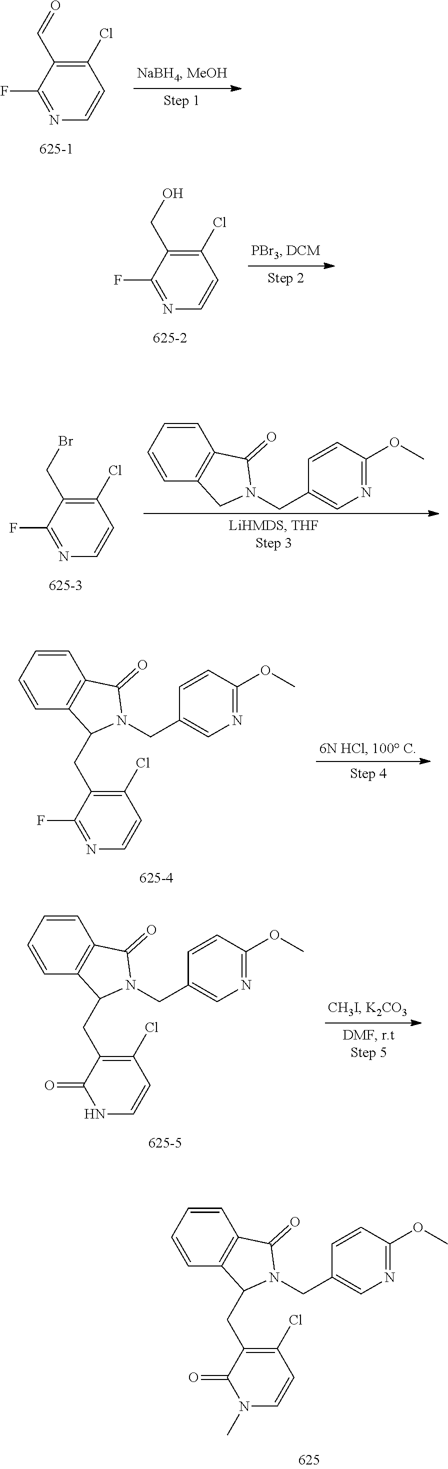

Step 1 was performed according to the procedure outlined for preparation of 164-2.

Step 2 was performed according to the procedure outlined for preparation of 217-3.

Step 3 was performed according to the procedure outlined for preparation of Int 12-2.

### Step 4: 3-((4-chloro-2-oxo-1,2-dihydropyridin-3-yl)methyl)-2-((6-methoxypyridin-3-yl)methyl)isoindolin-1-one (625-5)

The mixture of 625-4 (100 mg, 0.25 mmol) in 6N HCl (5 mL) was stirred for 16 h at 100 C, then quenched with NaHCO₃ (aq.) (5 mL) and extracted with ethyl acetate (2*10 mL). The combined organic layer was dried over anhydrous Na₂SO₄ and concentrated. The residue was purified by column chromatography on silica gel (eluting PE/EA=1/1) to give 625-5 (75 mg, 74%) as a white solid. MS (ESI) m/z 396 [M+H]⁺.

Step 5 was performed according to the procedure outlined for preparation of 381-4 to afford 625 (30 mg, 38%) as a white solid. MS (ESI) m/z 410 [M+H]⁺. ¹H NMR (400 MHz, CDCl₃) δ 7.91-7.82 (m, 2H), 7.52 (dd, J=8.5, 2.4 Hz, 1H), 7.49-7.40 (m, 2H), 7.28 (s, 1H), 7.23-7.18 (m, 1H), 6.65 (d, J=8.5 Hz, 1H), 6.29 (d, J=7.3 Hz, 1H), 5.26 (d, J=15.2 Hz, 1H), 4.95 (t, J=6.6 Hz, 1H), 4.27 (d, J=15.3 Hz, 1H), 3.89 (s, 3H), 3.54 (s, 3H), 3.26 (dd, J=13.4, 6.6 Hz, 1H), 3.00 (dd, J=13.4, 6.7 Hz, 1H).

### 3-((4-chloro-1-methyl-1H-pyrazol-5-yl)methyl)-2-((2-thioxo-2,3-dihydrobenzo[d]oxazol-6-yl)methyl)isoindolin-1-one (626)

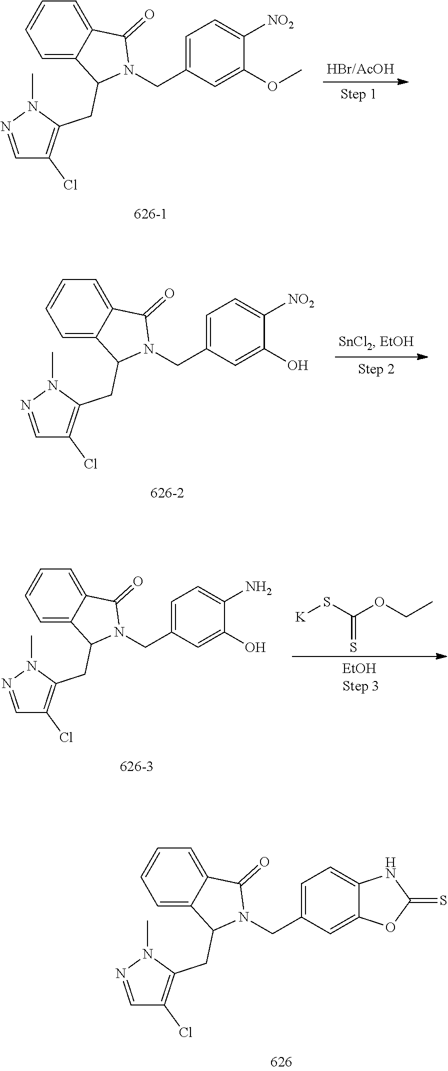

626-1 was performed according to the procedure outlined for preparation of 502.

Step 1 was performed according to the procedure outlined for preparation of 578.

Step 2. 2-[(4-amino-3-hydroxy-phenyl)methyl]-3-[(4-chloro-2-methyl-pyrazol-3-yl)methyl]isoindolin-1-one (626-3)

To a solution of 626-2 (500 mg, 1.21 mmol) in ethanol (10 mL) was added SnCl₂ (687 mg, 3.62 mmol) under N₂. The mixture was stirred for 2 h at 80° C.C. After the reaction was completed, the solid was filtered and the filtrate was concentrated. The residue was purified by silica gel flash column chromatography (PE/EA=2/1) to give 626-3 (300 mg, 64.9%) as a yellow oil. MS (ESI) m/z 383 [M+H]⁺.

### Step 3. 3-((4-chloro-1-methyl-1H-pyrazol-5-yl)methyl)-2-((2-thioxo-2,3-dihydrobenzo[d]oxazol-6-yl)methyl)isoindolin-1-one (626)

A mixture of 626-3 (40 mg, 0.10 mmol) and potassium O-ethyl carbonodithioate (18 mg, 0.12 mmol) in pyridine (10 mL) was refluxed for 2 h. Then the mixture was cooled to r.t., poured into a mixture of ice-water (40 mL) and concentrated HCl (4 mL). The solid was collected, washed with water, and purified by prep-HPLC to give 626 (15 mg, 33.9%) as a white solid. MS (ESI) m/z 425 [M+H]⁺. ¹H NMR (400 MHz, DMSO-d₆) δ 13.88 (s, 1H), 7.78-7.71 (m, 1H), 7.59-7.46 (m, 3H), 7.35 (s, 1H), 7.19 (d, J=8.0 Hz, 1H), 7.13 (d, J=8.0 Hz, 1H), 6.87-6.81 (m, 1H), 5.24 (d, J=15.2 Hz, 1H), 4.68 (t, J=7.2 Hz, 1H), 4.40 (d, J=15.2 Hz, 1H), 3.57 (s, 3H), 3.50 (dd, J=14.8, 6.0 Hz, 1H), 2.98 (dd, J=14.8, 8.4 Hz, 1H).

### 3-((4-chloro-1-methyl-1H-pyrazol-5-yl)methyl)-2-((3-methyl-[1,2,3]triazolo[1,5-a]pyridin-6-yl)methyl)isoindolin-1-one (627)

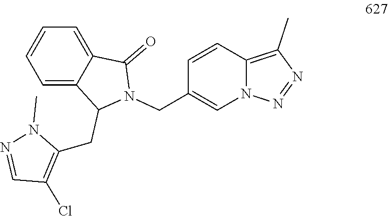 [SMILES: Cc1nnn2cc(CN3C(=O)c4ccccc4C3Cc3c(Cl)cnn3C)ccc12]

The title compound 627 was prepared according to the procedure described for compound 502 as a white solid (46 mg, 33.2%). MS (EST) m/z 407 [M+H]⁺. ¹H NMR (400 MHz, DMSO-d₆) δ 8.99 (s, 1H), 7.86 (dd, J=9.2, 1.0 Hz, 1H), 7.79-7.71 (m, 1H), 7.55-7.47 (m, 3H), 7.14 (dd, J=9.1, 1.4 Hz, 11H), 6.81-6.73 (m, 11H), 5.24 (d, J=15.3 Hz, 1H), 4.77 (dd, J=8.7, 5.4 Hz, 1H), 4.54 (d, J=15.4 Hz, 1H), 3.65 (dd, J=14.8, 5.5 Hz, 1H), 3.60 (s, 3H), 2.98 (dd, J=14.8, 8.8 Hz, 1H), 2.51 (s, 3H)).

The intermediate 627-6 was prepared as follows:

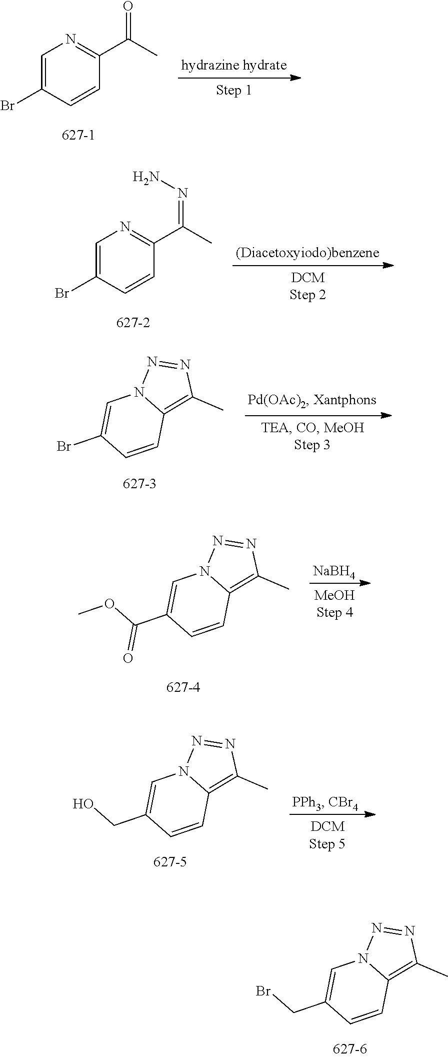

### Step 1. 1-(5-bromo-2-pyridyl)ethanone hydrazone (627-2)

The mixture of 1-(5-bromo-2-pyridyl)ethanone (500 mg, 2.50 mmol) in hydrazine hydrate (4.0 mL) was stirred for 2 h at 100° C. under N₂. The reaction mixture was concentrated to give 627-2 (500 mg, 93.5%) as a yellow oil. MS (ESI) m/z 214 [M+H]⁺.

### Step 2. 5-bromo-3-methyl-triazolo[1,5-a]pyridine (627-3)

To a solution of 627-2 (1.0 g, 4.67 mmol) in DCM (10 mL) was added (Diacetoxyiodo)benzene (1.5 g, 4.67 mmol) at r.t. After addition, the mixture was stirred for 1 h at 25° C. Then water (10 mL) was added and the mixture was extracted with EA (10 mL*3). The organics were combined and dried (Na₂SO₄) before concentration to dryness. The residue was purified by flash (PE/EA=5/1) to give 627-3 (680 mg, 68.6%) as a white solid. MS (ESI) m/z 212 [M+H]⁺.

Step 3 was performed according to the procedure outlined for the preparation of 382-1.

Step 4 was performed according to the procedure outlined for the preparation of 164-2.

Step 5 was performed according to the procedure outlined for the preparation of 164-3.

### 2-((2-aminothiazol-5-yl)methyl)-3-((4-chloro-1-methyl-1H-pyrazol-5-yl)methyl)isoindolin-1-one (628)

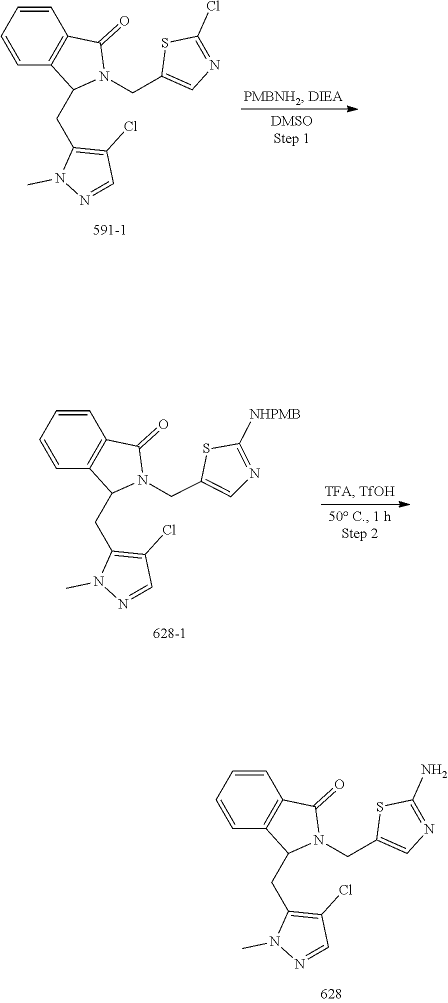

### Step 1. 3-((4-chloro-1-methyl-1H-pyrazol-5-yl)methyl)-2-((2-((4-methoxybenzyl)amino)thiazol-5-yl)methyl)isoindolin-1-one (628-1)

The mixture of 591-1 (500 mg, 1.28 mmol), PMBNH₂ (1.37 g, 10.00 mmol) and DIEA (2 mL) in DMSO (10 mL) was stirred at 100° C. for 16 h. The mixture was concentrated under vacuum. The residue was purified by HPLC to give 628-1 (20 mg, 3%) as a white solid. MS (ESI) m/z 494 [M+H]⁺.

Step 2 was performed according to the procedure outlined for step 2 of preparation of 4 to afford 628 (6 mg, 40.4%) as a white solid. MS (ESI) m/z 374 [M+H]⁺. ¹H NMR (400 MHz, DMSO-d₆) δ 7.73-7.67 (m, 1H), 7.56-7.48 (m, 3H), 6.93 (d, J=7.2 Hz, 1H), 6.88 (s, 2H), 6.81 (s, 1H), 5.06 (d, J=15.6 Hz, 1H), 4.71 (t, J=7.2 Hz, 1H), 4.31 (d, J=15.6 Hz, 1H), 3.65 (s, 3H), 3.51 (dd, J=14.8, 6.4 Hz, 1H), 3.01 (dd, J=14.8, 8.0 Hz, 1H).

### 2-((1H-pyrazol-4-yl)methyl)-3-((4-chloro-1-methyl-1H-pyrazol-5-yl)methyl)isoindolin-1-one (629)

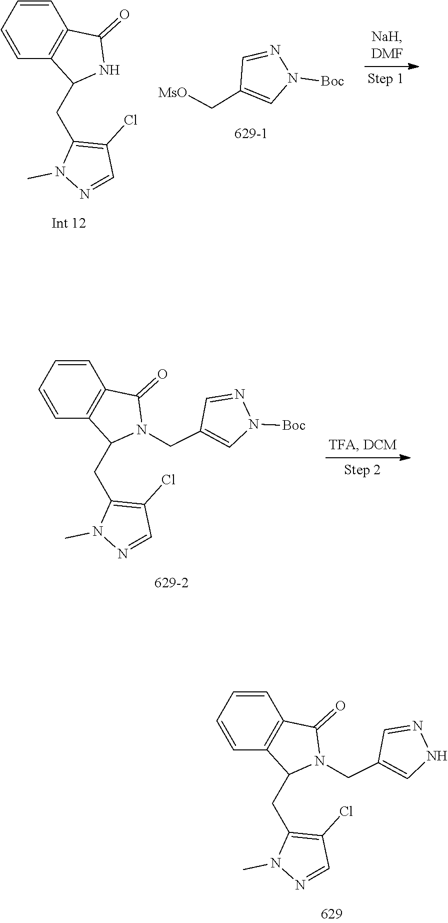 [SMILES: Cn1ncc(Cl)c1CC1NC(=O)c2ccccc21 ; CC(C)(C)OC(=O)n1cc(COS(C)(=O)=O)cn1 ; Cn1ncc(Cl)c1CC1c2ccccc2C(=O)N1Cc1cnn(C(=O)OC(C)(C)C)c1 ; Cn1ncc(Cl)c1CC1c2ccccc2C(=O)N1Cc1cn[nH]c1]

Step 1 was performed according to the procedure outlined for the preparation of 502.

Step 2 was performed according to the procedure outlined for step 3 of preparation of 12 to afford 629 (19 mg, 50%) as a white solid. MS (ESI) m/z 342 [M+H]⁺. ¹H NMR (400 MHz, CD₃OD) δ 7.85-7.76 (m, 1H), 7.57 (s, 2H), 7.56-7.45 (m, 3H), 6.82-6.80 (m, 1H), 5.20 (d, J=15.6 Hz, 1H), 4.84-4.79 (m, 1H), 4.33 (d, J=15.6 Hz, 1H), 3.57-3.52 (m, 4H), 2.95 (dd, J=14.8, 8.8 Hz, 1H).

### 3-((4-chloro-1-methyl-1H-pyrazol-5-yl)methyl)-2-((5-fluoro-1H-1,2,3-triazol-4-yl)methyl)isoindolin-1-one (630)

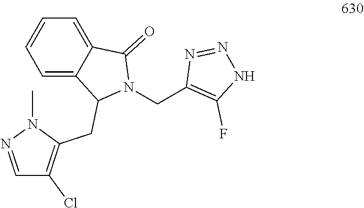 [SMILES: Cn1ncc(Cl)c1CC1c2ccccc2C(=O)N1Cc1nn[nH]c1F]

The title compound 630 was prepared according to the procedure described for compound 502 as a white solid (37 mg, 28%). MS (EST) m/z 361 [M+H]⁺. ¹H NMR (400 MHz, CD₃OD) δ 7.79 (d, J=6.8 Hz, 1H), 7.50-7.44 (m, 3H), 6.69 (d, J=7.2 Hz, 1H), 5.33 (d, J=15.6 Hz, 1H), 4.82-4.80 (m, 1H), 4.52 (d, J=16.0 Hz, 1H), 3.66 (dd, J=14.8, 5.6 Hz, 1H), 3.57 (s, 3H), 2.86 (dd, J=14.4, 9.6 Hz, 1H).

The intermediate 630-5 was prepared as follows:

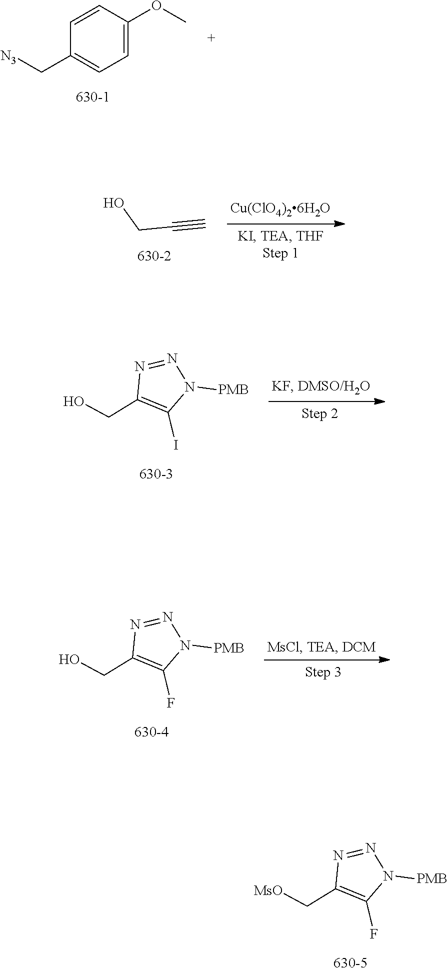

### Step 1. (5-iodo-1-(4-methoxybenzyl)-1H-1,2,3-triazol-4-yl)methanol (630-3)

To a solution of 1-(azidomethyl)-4-methoxybenzene (1.00 g, 6.13 mmol), Cu(ClO₄)₂·6H₂O (4.55 g, 12.26 mmol) and KI (4.07 g, 24.54 mmol) in THF (50 mL) was added TEA (1.24 g, 12.27 mmol) and prop-2-yn-1-ol (343 mg, 6.13 mmol) at r.t. The mixture was stirred at r.t. overnight under N₂. The mixture was poured into water (50 mL), and extracted with EA (50 mL*2). The combined organic layers were washed with NH₃H₂O (20 mL) and H₂O (20 mL), dried over Na₂SO₄, filtered, concentrated and purified by FCC (DCM/MeOH=20/1) to give 630-3 (1.7 g, 81%) as a white solid. MS (ESI) m/z 346 [M+H]⁺.

### Step 2. (5-fluoro-1-(4-methoxybenzyl)-1H-1,2,3-triazol-4-yl)methanol (630-4)

The mixture of (5-iodo-1-(4-methoxybenzyl)-1H-1,2,3-triazol-4-yl)methanol (900 mg, 2.6 mmol), KF (756 mg, 13.0 mmol) in DMSO/H₂O (8 mL/8 mL) was stirred at 160° C. for 10 min under the microwave. The mixture was poured into water (50 mL), extracted with EA (30 mL*3). The combined organic layers were separated, washed with water (30 mL*2) and brine (30 mL), dried over anhydrous Na₂SO₄, filtered and concentrated under vacuum. The residue was purified by FCC (PE/EA=1/1) to give 630-4 (440 mg, 71%) as a white solid. MS (ESI) m/z 238 [M+H]⁺.

Step 3 was performed according to the procedure outlined for the preparation of 132-2.

### 3-((4-chloro-1-methyl-1H-pyrazol-5-yl)methyl)-2-((2-thioxo-2,3-dihydrothiazol-5-yl)methyl)isoindolin-1-one (631)

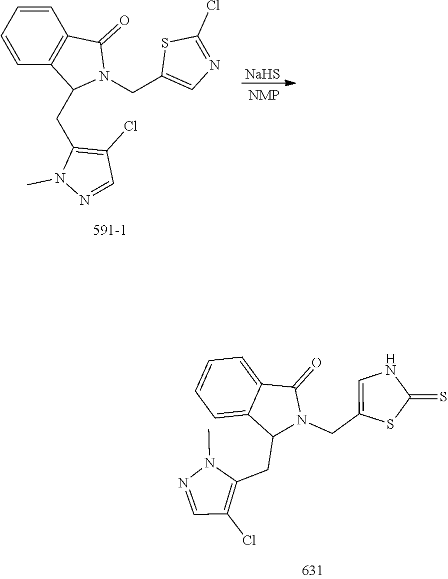

The solution of 591-1 (200 mg, 0.51 mmol) and NaHS (285 mg, 5.1 mmol) in NMP (5 mL) was stirred at 80° C. for 1 h. The mixture was poured into water (20 mL), extracted with EA (10 mL*2). The combined organic layers were washed with water (10 m*2) and brine (10 mL), dried over anhydrous Na₂SO₄, filtered and concentrated under vacuum. The residue was purified by prep-HPLC to give 631 (86 mg, 43%) as a white solid. MS (ESI) m/z 391 [M+H]⁺. ¹H NMR (400 MHz, DMSO-d₆) δ 13.11 (brs, 1H), 7.72-7.71 (m, 1H), 7.55-7.49 (m, 3H), 7.28 (s, 1H), 6.84 (d, J=7.2 Hz, 1H), 4.96 (d, J=15.2 Hz, 1H), 4.85 (dd, J=8.0, 5.2 Hz, 1H), 4.47 (d, J=15.6 Hz, 1H), 3.66 (s, 3H), 3.60 (dd, J=14.8, 5.6 Hz, 1H), 2.96 (dd, J=14.8, 8.4 Hz, 1H).

### 5-((1-((4-chloro-1-methyl-1H-pyrazol-5-yl)methyl)-3-oxoisoindolin-2-yl)methyl)-1,3,4-oxadiazol-2(3H)-one (632)

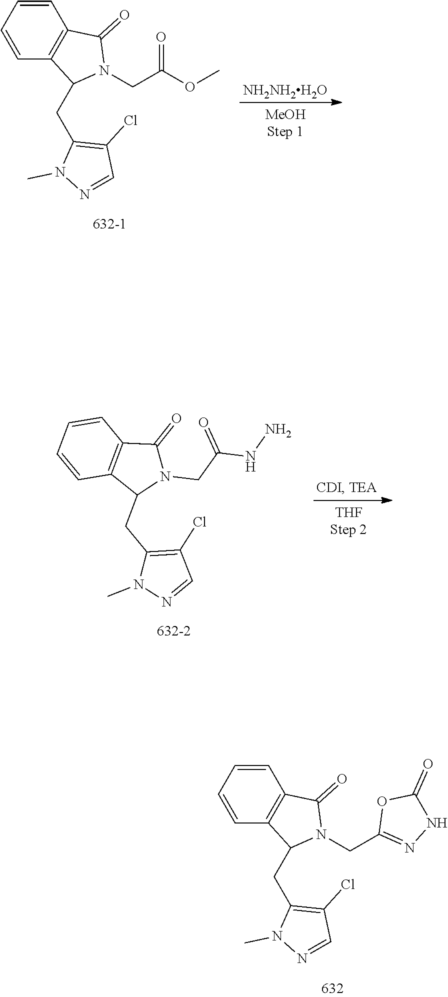

632-1 was performed according to the procedure outlined for the preparation of 502.

### Step 1. 2-(1-((4-chloro-1-methyl-1H-pyrazol-5-yl)methyl)-3-oxoisoindolin-2-yl)acetohydrazide (632-2)

The solution of 632-1 (80 mg, 0.24 mmol) and NH₂NH₂·H₂O (60 mg, 1.20 mmol) in MeOH (5 mL) was stirred at 70° C. for 6 h under N₂. The mixture was diluted with water (5 mL) and extracted with DCM (5 mL*3). The combined organic layers were washed with brine (5 mL), dried over anhydrous Na₂SO₄, filtered and concentrated. The residue was purified by pre-TLC (DCM/MeOH=10/1) to give 632-2 (55 mg, 69%) as a white solid. MS (ESI) m/z 334 [M+H]⁺.

### Step 2. 5-((1-((4-chloro-1-methyl-1H-pyrazol-5-yl)methyl)-3-oxoisoindolin-2-yl)methyl)-1,3,4-oxadiazol-2(3H)-one (632)

A solution of 632-1 (55 mg, 0.65 mmol), CDI (32 mg, 0.98 mmol) and TEA (20 mg, 0.98 mmol) in THF (5 mL) was stirred at r.t. overnight. The solvent was removed in vacuo and the residue was purified by prep-HPLC to give 632 (28 mg, 29%) as a white solid. MS (ESI) m/z 360 [M+H]⁺. ¹H NMR (400 MHz, DMSO-d₆) δ 12.29 (brs, 1H), 7.74 (d, J=6.8 Hz, 1H), 7.57-7.52 (m, 3H), 6.92 (d, J=7.2 Hz, 11H), 5.07-4.99 (m, 2H), 4.44 (d, J=16.4 Hz, 1H), 3.69 (s, 3H), 3.55 (dd, J=14.8, 5.6 Hz, 1H), 2.99 (dd, J=15.2, 8.4 Hz, 11H).

### (R)-3-((4-chloro-1-methyl-1H-pyrazol-5-yl)methyl)-2-(3-fluoro-4-methoxybenzyl)isoindolin-1-one (633) and (S)-3-((4-chloro-1-methyl-1H-pyrazol-5-yl)methyl)-2-(3-fluoro-4-methoxybenzyl)isoindolin-1-one (634)

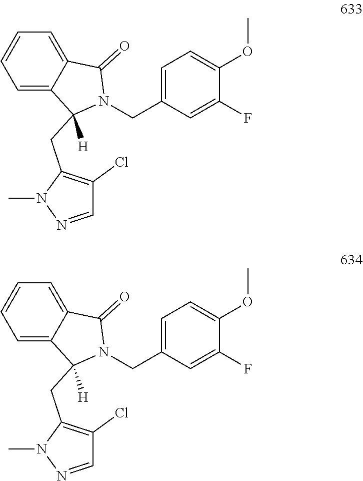 [SMILES: COc1ccc(CN2C(=O)c3ccccc3[C@H]2Cc2c(Cl)cnn2C)cc1F ; COc1ccc(CN2C(=O)c3ccccc3[C@@H]2Cc2c(Cl)cnn2C)cc1F]

Chiral separation of compound 517 by chiral prep-HPLC (column: CHIRALPAK IA; column size: 2 cm×25 cm, 5 μm; mobile phase A: MTBE; mobile phase B: MeOH:DCM) was conducted to afford two enantiomers: compound 633 and compound 634. One of the two enantiomers was a white solid, with RT=2.036 min (CHIRAL HPLC, column: CHIRALPAK IA-3; column size: 4.6*50 mm, 3 μm; mobile phase: MTBE (0.1% DEA):(MeOH:DCM=1:1)=90:10; flow: 1 mL/min), MS (ESI) m/z 400 [M+H]+, 1H NMR (400 MHz, CDCl₃) δ 7.91 (dt, J=7.5, 1.0 Hz, 1H), 7.51-7.40 (m, 3H), 7.04-6.85 (m, 3H), 6.66-6.62 (m, 1H), 5.31 (d, J=15.2 Hz, 1H), 4.67 (dd, J=9.1, 6.1 Hz, 1H), 4.12 (d, J=15.2 Hz, 1H), 3.87 (s, 3H), 3.47 (s, 3H), 3.37 (dd, J=14.5, 6.1 Hz, 1H), 2.67 (dd, J=14.5, 9.1 Hz, 1H). The other one of the two enantiomers was a white solid, with RT=2.496 min (CHIRAL HPLC, column: CHIRALPAK IA-3; column size: 4.6*50 mm, 3 μm; mobile phase: MTBE (0.1% DEA):(MeOH:DCM=1:1)=90:10; flow: 1 mL/min). MS (ESI) m/z 400 [M+H]+¹H NMR (400 MHz, CDCl₃) δ 7.91 (dt, J=7.6, 1.2 Hz, 1H), 7.50-7.40 (m, 3H), 7.04-6.85 (m, 3H), 6.67-6.60 (m, 1H), 5.31 (d, J=15.1 Hz, 1H), 4.67 (dd, J=9.1, 6.1 Hz, 1H), 4.12 (d, J=15.2 Hz, 1H), 3.87 (s, 3H), 3.47 (s, 3H), 3.37 (dd, J=14.5, 6.1 Hz, 11H), 2.67 (dd, J=14.5, 9.1 Hz, 11H).

### 3-((4-chloro-1-methyl-1H-pyrazol-5-yl)methyl)-2-((2-hydroxy-4-methylthiazol-5-yl)methyl)isoindolin-1-one (635)

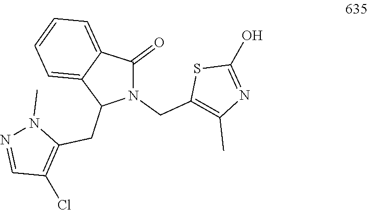 [SMILES: Cc1nc(O)sc1CN1C(=O)c2ccccc2C1Cc1c(Cl)cnn1C]

The title compound 635 was prepared according to the procedure described for compound 592 as a white solid (47 mg, 62.2%). MS (ESI) m/z 389 [M+H]⁺. ¹H NMR (400 MHz, DMSO-d₆) δ 11.04 (s, 1H), 7.71 (dd, J=6.4, 2.0 Hz, 1H), 7.57 (s, 1H), 7.55-7.49 (m, 2H), 6.85 (d, J=7.1 Hz, 11H), 4.91 (d, J=16.0 Hz, 11H), 4.82 (dd, J=8.4, 6.0 Hz, 11H), 4.29 (d, J=15.6 Hz, 1H), 3.63 (s, 3H), 3.48 (dd, J=14.8, 6.0 Hz, 1H), 2.97 (dd, J=14.8, 8.4 Hz, 1H), 2.06 (s, 3H).

### 3-((4-chloro-1-methyl-1H-pyrazol-5-yl)methyl)-2-(2-(6-oxo-1,6-dihydropyridin-3-yl)ethyl)isoindolin-1-one (636)

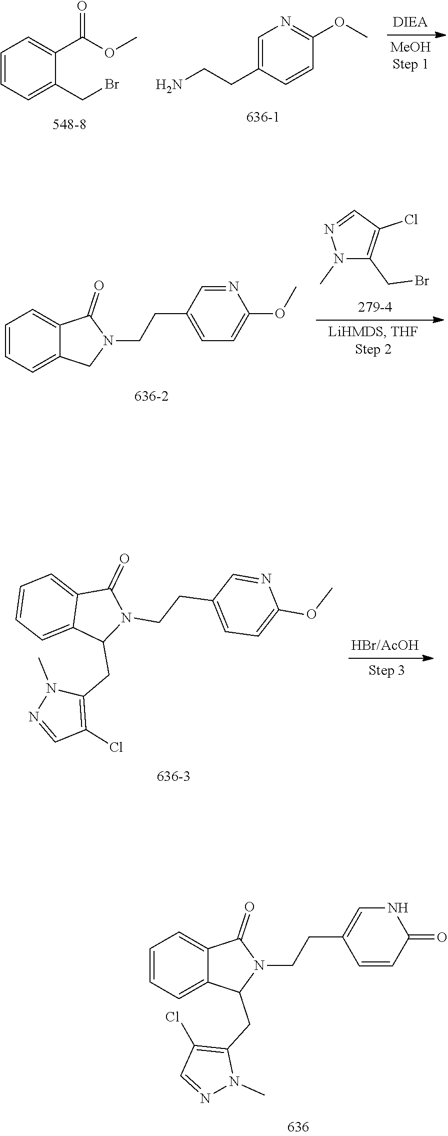

### Step 1. 2-(2-(6-methoxypyridin-3-yl)ethyl)isoindolin-1-one (636-2)

To a solution of 2-(6-methoxypyridin-3-yl)ethan-1-amine (800 mg, 5.3 mmol) and DIEA (2.3 mL, 13.2 mmol) in MeOH (20 mL) was added methyl 2-(bromomethyl)benzoate (1.1 g, 4.7 mmol) at r.t. The mixture was stirred at 60° C. overnight. The mixture was poured into water (100 mL), and extracted with EA (30 mL*3) and the organic layer was concentrated. The residue was triturated with PE (5 mL) to give 636-2 (900 mg, 64%) as a white solid. MS (ESI) m/z 269 [M+H]⁺.

Step 2 was performed according to the procedure outlined for the preparation of Int 12-2.

Step 3 was performed according to the procedure outlined for the preparation of 578 to give 636 (18 mg, 9%) as a white solid. MS (ESI) m/z 383 [M+H]⁺. ¹H NMR (400 MHz, DMSO-d₆) δ 7.69-7.61 (m, 1H), 7.56 (s, 1H), 7.53-7.46 (m, 2H), 7.33 (dd, J=9.2, 2.8 Hz, 1H), 7.14 (d, J=2.0 Hz, 1H), 6.91-6.83 (m, 1H), 6.25 (d, J=9.2 Hz, 1H), 4.88 (dd, J=8.4, 6.4 Hz, 1H), 4.04-3.99 (m, 1H), 3.67 (s, 3H), 3.50 (dd, J=14.4, 5.6 Hz, 1H), 3.37-3.31 (m, 1H), 2.90 (dd, J=14.8, 8.4 Hz, 1H), 2.68-2.55 (m, 2H).

### 2-((1H-1,2,3-triazol-4-yl)methyl)-3-((4-chloro-1-methyl-1H-pyrazol-5-yl)methyl)isoindolin-1-one (637)

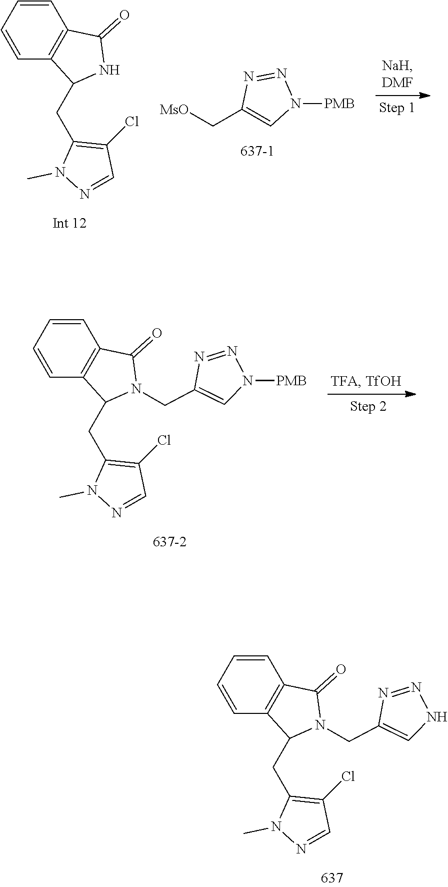

Step 1 was performed according to the procedure outlined for the preparation of 502.

Step 2 was performed according to the procedure outlined for step 2 for the preparation of 4 to give 637 (13 mg, 11.2%) as a white solid. MS (ESI) m/z 343 [M+H]⁺. ¹H NMR (400 MHz, DMSO-d₆) δ 7.75 (s, 1H), 7.73-7.66 (m, 1H), 7.55-7.49 (m, 3H), 6.84-6.82 (m, 1H), 6.07 (brs, 1H), 5.22 (d, J=15.2 Hz, 1H), 4.80 (dd, J=8.4, 5.6 Hz, 1H), 4.49 (d, J=15.6 Hz, 1H), 3.63 (s, 3H), 3.57 (dd, J=14.8, 6.0 Hz, 1H), 2.96 (dd, J=14.8, 8.8 Hz, 1H).

### 5-((6-cyclopropyl-2-(4-methoxybenzyl)-3-oxoisoindolin-1-yl)methyl)-6-methylpyrimidine-4-carbonitrile (638)

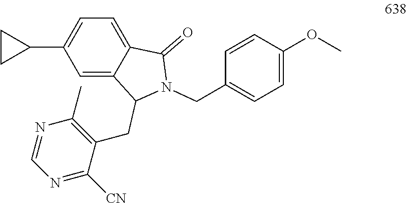 [SMILES: COc1ccc(CN2C(=O)c3ccc(C4CC4)cc3C2Cc2c(C)ncnc2C#N)cc1]

The title compound 638 was prepared according to the procedure described for 597 as a white solid (10 mg, 23%). MS (EST) m/z 425 [M+H]⁺. ¹H NMR (400 MHz, CDCl₃) δ 9.15 (s, 1H), 7.80 (d, J=7.8 Hz, 1H), 7.80 (d, J=8.0 Hz, 1H), 7.17 (d, J=8.0 Hz, 2H), 6.85 (d, J=8.4 Hz, 2H), 6.00 (s, 1H), 5.28 (d, J=15.3 Hz, 1H), 4.63 (dd, J=9.2, 6.1 Hz, 1H), 4.24 (d, J=15.2 Hz, 1H), 3.78 (s, 3H), 3.58 (dd, J=13.8, 5.9 Hz, 1i), 2.79 (dd, J=13.9, 9.2 Hz, 1i), 2.25 (s, 3H), 1.87-1.79 (m, 1H), 1.02-0.92 (m, 2H), 0.66-0.43 (m, 2H).

### 5-((6-chloro-2-((6-methoxypyridin-3-yl)methyl)-3-oxoisoindolin-1-yl)methyl)-6-methylpyrimidine-4-carbonitrile (639)

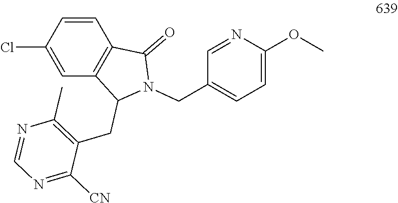 [SMILES: COc1ccc(CN2C(=O)c3ccc(Cl)cc3C2Cc2c(C)ncnc2C#N)cn1]

The title compound 639 was prepared according to the procedure described for 597 as a white solid (3 mg, 4.1%). MS (ESI) m/z 420 [M+H]⁺. ¹H NMR (400 MHz, CDCl₃) δ 9.19 (s, 1), 8.05 (d, J=2.2 Hz, 1H), 7.87 (d, J=8.1 Hz, 1H), 7.55-7.48 (m, 2H), 6.72 (d, J=8.6 Hz, 1), 6.51 (s, 11H), 5.20 (d, J=15.4 Hz, 1H), 4.72 (dd, J=9.2, 6.0 Hz, 1H), 4.31 (d, J=15.4 Hz, 1H), 3.92 (s, 3H), 3.59 (dd, J=14.1, 5.9 Hz, 1H), 2.86 (dd, J=14.1, 9.2 Hz, 1H), 2.35 (s, 3H).

### 5-((6-cyclopropyl-2-((6-methoxypyridin-3-yl)methyl)-3-oxoisoindolin-1-yl)methyl)-6-methylpyrimidine-4-carbonitrile (640)

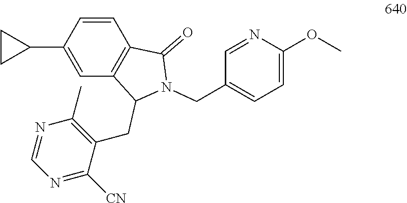 [SMILES: COc1ccc(CN2C(=O)c3ccc(C4CC4)cc3C2Cc2c(C)ncnc2C#N)cn1]

The title compound 640 was prepared according to the procedure described for 595 as a white solid (50 mg, 26%). MS (ESI) m/z 426 [M+H]⁺. ¹H NMR (400 MHz, CDCl₃) δ 9.16 (s, 1H), 8.10 (d, J=2.0 Hz, 1H), 7.79 (d, J=7.9 Hz, 1H), 7.59 (dd, J=8.6, 2.4 Hz, 1H), 7.30-7.19 (m, 1H), 6.72 (d, J=8.5 Hz, 1H), 5.99 (s, 1H), 5.21 (dd, J=15.4, 8.3 Hz, 1H), 4.65 (dd, J=9.4, 5.6 Hz, 1H), 4.34 (d, J=15.4 Hz, 1H), 3.93 (s, 3H), 3.62 (dd, J=13.8, 5.6 Hz, 1H), 2.78 (dd, J=13.8, 9.6 Hz, 1H), 2.28 (s, 3H), 1.86-1.77 (m, 1H), 1.00-0.94 (m, 2H), 0.61-0.44 (m, 2H).

### 3-((4-chloro-1-methyl-1H-pyrazol-5-yl)methyl)-2-(4-methoxybenzyl)-2,3-dihydrobenzo[d]isothiazole 1,1-dioxide (641)

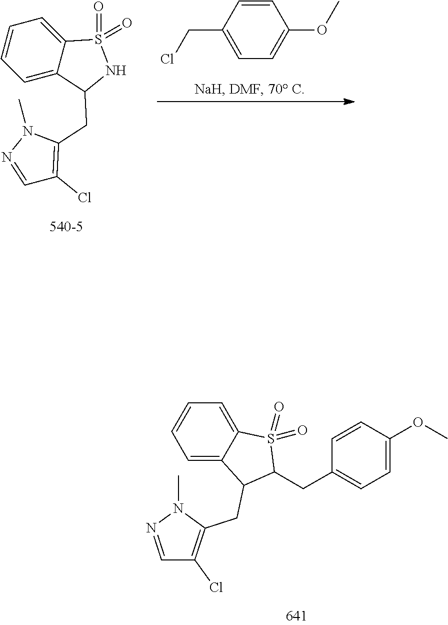

To a solution of 540-5 (158 mg, 0.53 mmol) in dried DMF (2 mL) was added NaH (23 mg, 0.58 mmol, 60% in oil) in portions at 0′C under N₂. The mixture was stirred for 30 min, and then added PMBCl (125 mg, 0.8 mmol). The mixture was stirred at 70 C for 2 h, then quenched with water (3 mL) and extracted with ethyl acetate (10 mL*2). The combined organic layer was washed with water (10 mL) and brine (10 mL), dried over Na₂SO₄, filtered, and concentrated in vacuo. The residue was purified by column chromatography on silica gel (PE/EA=2/1 to 1/1) to give 641 (46 mg, 20%) as a waxy solid. MS (ESI) m/z 418 [M+H]⁺. ¹H NMR (400 MHz, CDCl₃) δ 7.86 (d, J=7.5 Hz, 1H), 7.58-7.47 (m, 2H), 7.40 (s, 1H), 7.22-7.14 (m, 2H), 6.86-6.82 (m, 2H), 6.79 (d, J=7.6 Hz, 1H), 4.58 (t, J=7.6 Hz, 1H), 4.52 (d, J=14.8 Hz, 1H), 4.08 (d, J=14.6 Hz, 1H), 3.79 (s, 3H), 3.52 (s, 3H), 3.23 (dd, J=14.6, 7.8 Hz, 1H), 2.96 (dd, J=14.6, 7.4 Hz, 1H).

### 3-((4-chloro-1-methyl-1H-pyrazol-5-yl)methyl)-2-((6-methoxypyridin-3-yl)methyl)-2,3-dihydrobenzo[d]isothiazole 1,1-dioxide (642)

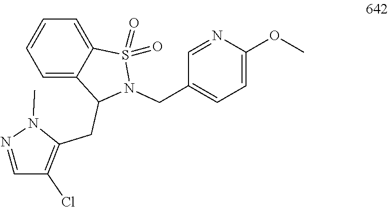 [SMILES: COc1ccc(CN2C(Cc3c(Cl)cnn3C)c3ccccc3S2(=O)=O)cn1]

The title compound 642 was prepared according to the procedure described for 641 as a white solid (32 mg, 15%). MS (ESI) m/z 419 [M+H]⁺. ¹H NMR (400 MHz, CDCl₃) δ 7.99 (d, J=2.3 Hz, 1H), 7.85 (dd, J=6.7, 1.9 Hz, 1H), 7.62-7.47 (m, 3H), 7.41 (s, 1H), 6.91 (dd, J=6.8, 1.7 Hz, 1H), 6.68 (d, J=8.6 Hz, 1H), 4.59 (dd, J=8.3, 6.7 Hz, 1H), 4.42 (d, J=14.9 Hz, 1H), 4.04 (d, J=14.9 Hz, 1H), 3.94 (s, 3H), 3.60 (s, 3H), 3.22 (dd, J=14.7, 8.4 Hz, 1H), 3.01 (dd, J=14.7, 6.7 Hz, 1H).

### 2-((1-((4-chloro-1-methyl-1H-pyrazol-5-yl)methyl)-3-oxoisoindolin-2-yl)methyl-d)-5-oxa-7-azaspiro[3.4]octan-6-one (643)

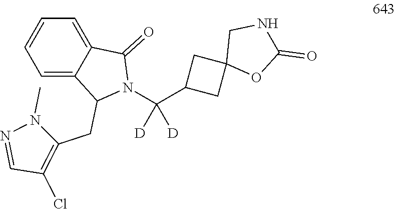 [SMILES: [2H]C([2H])(C1CC2(CNC(=O)O2)C1)N1C(=O)c2ccccc2C1Cc1c(Cl)cnn1C]

The title compound 643 was prepared according to the procedure described for compound 307 as a white solid (39 mg, 19%). MS (ESI) m/z 403 [M+H]⁺. ¹H NMR (400 MHz, DMSO-d₆) δ 7.72-7.63 (m, 1H), 7.55 (s, 1H), 7.53-7.44 (m, 3H), 6.83-6.74 (m, 1H), 4.88 (dd, J=8.6, 5.6 Hz, 1H), 3.64 (s, 3H), 3.55 (dd, J=14.8, 5.7 Hz, 1H), 3.50 (s, 2H), 2.87 (dd, J=14.8, 8.7 Hz, 1H), 2.43-2.33 (m, 1H), 2.32-2.12 (m, 3H), 2.12-2.04 (m, 1H).

The intermediate 643-3 was prepared as follows:

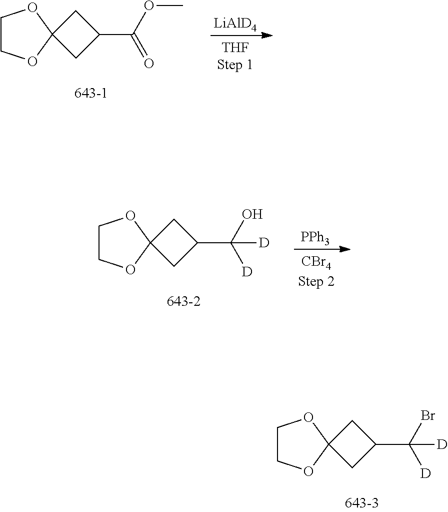

Step 1 was performed according to the procedure outlined for the preparation of 279-3.

Step 2 was performed according to the procedure outlined for the preparation of 164-3.

### Example II. NAMPT Enzymatic and Cellular Assays

### A. NMN Fluorescence Biochemical Assay

Certain compounds described herein were assayed for their ability to stimulate the synthesis of nicotinamide mononucleotide (NMN) by human enzyme NAMPT (full length). The enzymatic assay was performed in a 384-well plate using a TMD buffer (50 mM Tris-HCl, 10 mM MgCl2, 2 mM DTT, pH 7.5). Purified NAMPT (100 nM) was pre-incubated with 0.2 μL. DMSO solution of the test compound (with a series of concentrations) in 10 μL TMD buffer for 15 min at 37° C. The reaction was initiated by adding 10 μL solution of NAM (50 μM), PRPP (100 μM), and ATP (4 mM) in TMD buffer. After 1 h of incubation at 37 T, an aliquot (20 μL) of the NMN-containing sample was sequentially mixed with 5 μL of 20% acetophenone (in DMSO) and 5 μL of 2M KOH. The mixture was placed on ice for 10 min. Next, 22.5 μL of 100% formic acid was added to each sample, vortexed, and then incubated at 37° C. for 20 min. Fluorescence (Ex/Em=382/445 nm) was measured using an Envision plate reader. The potency measurements for the test compounds were quantified and represented as EC50 (concentration of a compound that gives half-maximal activation) and Emax (fold change of a compound's maximal activation compared to DMSO).

### B. Cellular NAD+ Modulation Assay.

Certain compounds described herein were also assayed for their ability to stimulate the endogenous NAMPT in a native cellular environment in a cellular NAD⁺ modulation assay. U2OS cells were grown in McCoy's 5A medium with 10% fetal bovine serum, in a humidified incubator with an atmosphere of 95% air and 5% CO₂ at 37° C. The assays were initiated by plating 30 μL of U2OS cells in a culture medium with 10% fetal bovine serum, at a density of 3000 cells per well in a Corning® 384-well White/Clear Bottom Polystyrene Microplate. The plates were incubated in 37T incubators for a period of 16 hours. Test compounds with a series of concentrations in DMSO were added to the plates in a volume of 150 nL using Echo Liquid handlers. The plates are then incubated for 24 hours. The medium was removed from the wells, then 10 μL PBS was added to each well. To each well of cells in 10 μL of PBS, 10 μL of base solution with 1% DTAB was added. The plate was briefly mixed on a plate shaker to ensure homogeneity and cell lysis (5 mins). 10p L of 0.4N HCl was added per well. The plate was coved, and all samples were incubated for 15 minutes at 60° C. Then the plate was equilibrated for 10 minutes at room temperature. 10 μL of 0.5M Trizma® base was added to each well of acid-treated cells to neutralize the acid. Finally, 30 μL of NAD/NADH-Glo™ Detection Reagent was added. The plate was gently shaken to mix the samples. The plate was incubated for 40 minutes at room temperature. Luminescence was recorded using a luminometer. The potency measurements for the test compounds were quantified and represented as EC50 (concentration of a compound that gives half-maximal activation) and Emax (fold change of a compound's maximal activation compared to DMSO).

Activities of the compounds as tested in the above-described two assays are summarized in Table 2. In Table 2, the levels of activities (EC50 and Emax for the enzymatic assay and the cellular assay, respectively) are provided as follows:

EnzymaticEC50: A<0.5 μM≤B<5 μM≤C≤5 μM≤D

Enzymatic Emax: A>3≥B>2≥C

Cellular EC50: A<0.5 μM≤B<5 μM≥C<50 μM≤D

Cellular Emax: A>1.5≥B>1.2≥C

TABLE 2

| | Enzymatic | Enzymatic | Cellular | Cellular |
|---|---|---|---|---|
| Cmpd No. | EC50 | Emax | EC50 | Emax |
| 1 | C | A | B | A |
| 2 | B | A | B | A |
| 3 | C | B | C | B |
| 4 | C | B | C | B |
| 5 | C | A | B | B |
| 6 | C | B | | |
| 7 | D | B | C | B |
| 8 | C | B | | |
| 9 | B | B | B | A |
| 10 | C | B | | |
| 11 | C | B | C | B |
| 12 | C | B | C | B |
| 13 | D | B | | |
| 14 | B | B | B | B |
| 15 | B | B | A | C |
| 16 | C | A | B | C |
| 17 | B | B | | |
| 18 | B | B | | |
| 19 | C | A | B | B |
| 20 | B | A | B | A |
| 21 | B | B | C | B |
| 22 | B | B | | |
| 23 | C | A | B | A |
| 24 | C | A | | |
| 25 | C | A | C | A |
| 26 | B | A | A | C |
| 27 | C | A | C | A |
| 28 | C | A | | |
| 29 | B | A | C | A |
| 30 | B | A | | |
| 31 | B | A | B | A |
| 32 | C | A | | |
| 33 | C | A | C | B |
| 34 | B | B | B | B |
| 35 | B | A | B | B |
| 36 | B | A | B | B |
| 37 | C | A | B | C |
| 38 | C | A | C | C |
| 39 | C | B | C | B |
| 40 | C | B | C | B |
| 41 | B | A | B | B |
| 42 | C | A | C | A |
| 43 | C | A | C | B |
| 44 | C | B | C | B |
| 45 | C | B | B | B |
| 46 | D | B | C | A |
| 47 | C | B | C | A |
| 48 | C | B | B | A |
| 49 | C | B | C | A |
| Compound 50/51 (RT = | C | B | A | C |
| 5.19 min) | | | | |
| Compound 50/51 (RT = | C | B | C | B |
| 5.41 min) | | | | |
| 52 | C | B | B | A |
| 53 | C | B | C | B |
| 54 | C | A | B | A |
| 55 | C | B | C | B |
| 56 | C | A | B | A |
| Compound 57/58 (RT = | C | B | B | B |
| 2.98 min) | | | | |
| Compound 57/58 (RT = | B | C | C | B |
| 3.27 min) | | | | |
| Compound 59/60 (RT = | C | A | C | B |
| 4.21 min) | | | | |
| Compound 59/60 (RT = | C | B | C | C |
| 4.34 min) | | | | |
| Mixture of 61 and 62 | C | A | | |
| Compound 63/64 (RT = | C | A | B | A |
| 3.54 min) | | | | |
| Compound 63/64 (RT = | C | C | | |
| 3.59 min) | | | | |
| Compound 65/66 (RT = | C | A | B | A |
| 2.09 min) | | | | |
| Compound 65/66 (RT = | B | C | | |
| 2.21 min) | | | | |
| 67 | C | B | C | B |
| Compound 68/69 (RT = | C | B | C | A |
| 2.69 min) | | | | |
| Compound 68/69 (RT = | C | C | | |
| 2.83 min) | | | | |
| Compound 70/71 (RT = | C | B | C | B |
| 2.85 min) | | | | |
| Compound 70/71 (RT = | C | C | | |
| 3.02 min) | | | | |
| 72 | C | A | B | A |
| Compound 73/74 (RT = | C | B | C | A |
| 3.760 min) | | | | |
| Compound 73/74 (RT = | B | A | B | A |
| 4.563 min) | | | | |
| Compound 75/76 (RT = | D | B | | |
| 2.25 min) | | | | |
| Compound 75/76 (RT = | C | A | B | A |
| 2.45 min) | | | | |
| Compound 77/78 (RT = | B | A | C | B |
| 3.95 min) | | | | |
| Compound 77/78 (RT = | B | C | | |
| 4.10 min) | | | | |
| 79 | C | A | A | C |
| Mixture of 80-1 and | B | A | C | B |
| 80-2 | | | | |
| 81 | C | A | B | B |
| 82 | C | B | B | B |
| Mixture of 83 and 84 | C | B | | |
| Mixture of 85 and 86 | B | B | | |
| Mixture of 87 and 88 | B | B | | |
| 89 | C | B | C | B |
| Compound 90/91 (RT = | C | B | | |
| 2.29 min) | | | | |
| Compound 90/91 (RT = | D | C | | |
| 2.45 min) | | | | |
| 92 | C | B | | |
| Compound 93/94 (RT= | B | B | C | A |
| 2.56 min) | | | | |
| Compound 93/94 (RT = | C | C | | |
| 2.80 min) | | | | |
| 95 | B | B | C | A |
| 96 | C | A | B | A |
| 97 | B | A | B | A |
| Mixture of 98 and 99 | B | A | B | A |
| Compound 100/101 | B | B | B | A |
| (RT = 4.58 min) | | | | |
| Compound 100/101 | B | C | | |
| (RT = 4.80 min) | | | | |
| Compound 102/103 | C | A | B | C |
| (RT = 3.37 min) | | | | |
| Compound 102/103 | B | A | C | B |
| (RT = 2.99 min) | | | | |
| 104 | C | B | C | A |
| 105 | C | B | B | A |
| 106 | B | A | B | B |
| Compound 107/108 | C | B | C | C |
| (RT = 2.417 min) | | | | |
| Compound 107/108 | B | A | B | A |
| (RT = 3.042 min) | | | | |
| Compound 109/110 | C | B | C | B |
| (RT = 2.19 min) | | | | |
| Compound 109/110 | C | C | | |
| (RT = 2.46 min) | | | | |
| Compound 111/112 | C | B | B | A |
| (RT = 3.90 min) | | | | |
| Compound 111/112 | D | D | | |
| (RT = 4.08 min) | | | | |
| 113 | B | B | B | B |
| 114 | B | B | C | A |
| 115 | C | A | C | B |
| 116 | A | B | B | A |
| Compound 117/118 | B | C | C | A |
| (RT = 5.06 min) | | | | |
| Compound 117/118 | B | A | B | A |
| (RT = 4.94 min) | | | | |
| Compound 119/120 | B | B | | |
| (RT = 3.35 min) | | | | |
| Compound 119/120 | B | B | | |
| (RT = 3.54 min) | | | | |
| Compound 121/122 | D | D | | |
| (RT = 3.94 min) | | | | |
| Compound 121/122 | B | B | | |
| (RT = 4.04 min) | | | | |
| 123 | B | A | B | A |
| 124 | A | A | B | A |
| 125 | C | A | B | A |
| Compound 126/127 | B | A | B | A |
| (Int 10/Int 11: RT = | | | | |
| 1.728 min as a starting | | | | |
| material) | | | | |
| Compound 126/127 | B | B | B | A |
| (Int 10/Int 11: RT = | | | | |
| 1.969 min as a starting | | | | |
| material) | | | | |
| 128 | C | B | B | A |
| 129 | B | A | B | A |
| 130 | C | B | B | A |
| 131 | B | A | B | A |
| Compound 132/133 | C | A | C | B |
| (RT = 2.07 min) | | | | |
| Compound 132/133 | C | C | | |
| (RT = 2.53 min) | | | | |
| 134 | A | A | B | A |
| Compound 135/136 | B | B | C | B |
| (RT = 2.075 min) | | | | |
| Compound 135/136 | A | A | B | A |
| (RT = 2.971 min) | | | | |
| 137 | B | B | A | A |
| Compound 138/139 | C | B | B | A |
| (Int 10/Int11: RT = | | | | |
| 1.728 min as a starting | | | | |
| material) | | | | |
| Compound 138/139 | B | A | B | A |
| (Int 10/Int11: RT = | | | | |
| 1.969 min as a starting | | | | |
| material) | | | | |
| 140 | C | A | B | A |
| 141 | B | B | B | A |
| 142 | A | B | B | A |
| 143 | C | A | B | A |
| Compound 144/145 | C | B | | |
| (Int 10/Int11: RT = | | | | |
| 1.728 min as a starting | | | | |
| material) | | | | |
| Compound 144/145 | C | A | B | A |
| (Int 10/Int11: RT = | | | | |
| 1.969 min as a starting | | | | |
| material) | | | | |
| 146 | A | A | A | A |
| 147 | A | A | A | A |
| 148 | B | B | B | A |
| 149 | B | B | B | A |
| 150 | C | A | C | A |
| 151 | C | A | B | A |
| Mixture of 152 and | C | C | | |
| 153/mixture of 154 | | | | |
| and 155 (RT = 3.24 | | | | |
| min) | | | | |
| Mixture of 152 and | C | B | C | A |
| 153/mixture of 154 | | | | |
| and 155 (RT = 3.68 | | | | |
| min) | | | | |
| 156 | C | A | B | A |
| 157 | A | A | B | A |
| 158 | C | A | B | A |
| 159 | A | A | A | A |
| 160 | B | B | B | A |
| 161 | B | A | B | A |
| 162 | B | B | C | A |
| 163 | C | A | B | A |
| 164 | A | B | A | A |
| 165 | B | B | | |
| 166 | B | B | B | B |
| 167 | B | B | B | B |
| 168 | B | A | B | A |
| 169 | A | B | A | A |
| 170 | A | A | A | A |
| 171 | C | C | | |
| 172 | A | B | A | A |
| 173 | B | B | B | A |
| 174 | A | B | B | A |
| 175 | A | B | A | A |
| 176 | A | A | B | A |
| 177 | C | C | | |
| 178 | A | A | A | A |
| 179 | C | B | | |
| 180 | B | B | | |
| 181 | C | B | | |
| 182 | B | A | B | A |
| 183 | B | B | | |
| 184 | B | B | | |
| 185 | C | B | | |
| 186 | C | B | B | A |
| 187 | B | B | B | A |
| 188 | C | B | B | A |
| 189 | B | B | B | A |
| 190 | A | B | A | A |
| 191 | B | A | B | A |
| Compound 192/193 | B | C | | |
| (RT = 1.350 min) | | | | |
| Compound 192/193 | B | A | B | A |
| (RT = 1.680 min) | | | | |
| 194 | B | B | | |
| 195 | A | B | A | B |
| 196 | B | B | | |
| 197 | B | B | B | A |
| 198 | B | B | B | A |
| 199 | A | A | A | A |
| 200 | A | B | A | A |
| 201 | B | A | A | A |
| 202 | B | A | A | A |
| 203 | B | A | A | A |
| 204 | B | B | | |
| 205 | A | A | A | A |
| Compound 206/207 | C | B | | |
| (RT = 1.087 min) | | | | |
| Compound 206/207 | A | A | A | A |
| (RT = 1.302 min) | | | | |
| 208 | B | B | | |
| 209 | A | A | A | A |
| 210 | B | A | B | A |
| 211 | A | A | A | A |
| 212 | A | A | A | A |
| 213 | B | A | | |
| 214 | B | A | A | A |
| 215 | B | A | A | A |
| 216 | B | A | | |
| 217 | B | A | B | A |
| 218 | B | A | | |
| 219 | B | B | | |
| 220 | B | B | | |
| 221 | A | A | A | A |
| 222 | C | B | | |
| 223 | A | A | B | A |
| 224 | A | A | B | A |
| 225 | B | A | B | A |
| 226 | C | B | | |
| 227 | B | A | B | A |
| 228 | A | B | A | A |
| Compound 229/230 | A | A | A | B |
| (RT = 7.184 min) | | | | |
| Compound 229/230 | B | A | | |
| (RT = 8.249 min) | | | | |
| 231 | C | A | B | A |
| 232 | A | A | A | A |
| 233 | B | B | | |
| 234 | C | A | B | A |
| Mixture of 235 and | C | C | | |
| 236/mixture of 237 | | | | |
| and 238 (RT = 3.54 | | | | |
| min) | | | | |
| Mixture of 235 and | B | A | B | A |
| 236/mixture of 237 | | | | |
| and 238 (RT = 3.63 | | | | |
| min) | | | | |
| 239 | C | B | | |
| 240 | A | B | A | B |
| 241 | B | A | B | B |
| 242 | C | A | B | A |
| 243 | C | A | B | A |
| 244 | C | B | | |
| 245 | C | B | | |
| 246 | C | B | | |
| 247 | C | B | | |
| 248 | B | A | | |
| 249 | A | A | | |
| 250 | A | B | | |
| 251 | A | A | | |
| 252 | B | B | | |
| 253 | C | A | | |
| 254 | B | B | | |
| 255 | B | A | | |
| 256 | C | A | | |
| 257 | B | A | A | A |
| Compound 258/259 | C | C | | |
| (RT = 1.809 min) | | | | |
| Compound 258/259 | B | A | A | A |
| (RT = 2.246 min) | | | | |
| 260 | B | B | | |
| 261 | B | A | | |
| 262 | B | A | | |
| 263 | A | A | | |
| 264 | B | B | | |
| 265 | C | A | | |
| 266 | C | A | | |
| 267 | B | A | | |
| 268 | B | A | | |
| 269 | C | B | | |
| 270 | A | A | A | B |
| 271 | B | A | B | A |
| 272 | B | A | A | A |
| 273 | C | B | B | B |
| 274 | C | B | C | B |
| 275 | C | B | B | A |
| 276 | A | A | A | A |
| 277 | B | A | A | A |
| 278 | A | A | A | A |
| 279 | A | B | A | A |
| 280 | C | B | | |
| 281 | C | C | | |
| 282 | A | B | A | B |
| 283 | A | A | A | B |
| 284 | A | A | A | B |
| 285 | A | B | | |
| 286 | A | B | | |
| 287 | B | B | | |
| 288 | C | C | | |
| 289 | C | C | C | B |
| 290 | D | C | | |
| 291 | A | C | A | B |
| 292 | A | C | A | C |
| 293 | A | C | C | A |
| 294 | C | C | C | A |
| 295 | B | C | B | B |
| 296 | B | C | B | B |
| 297 | B | C | B | B |
| 298 | B | C | B | B |
| 299 | B | C | B | B |
| 300 | C | C | C | B |
| 301 | C | C | C | B |
| Compound 302/303 | B | C | C | B |
| (RT = 4.82 min) | | | | |
| Compound 302/303 | B | C | | |
| (RT = 4.16 min) | | | | |
| 304 | B | C | B | B |
| 305 | C | C | | |
| 306 | C | C | | |
| 307 | B | B | A | A |
| 308 | A | B | A | A |
| 309 | A | B | A | A |
| 310 | B | B | A | A |
| 311 | B | B | A | A |
| 312 | A | B | A | A |
| 313 | C | C | | |
| 314 | A | B | A | A |
| 315 | B | A | C | B |
| 316 | A | B | A | B |
| 317 | A | C | A | B |
| 318 | A | A | A | A |
| Compound 319/320 | C | C | | |
| (RT = 2.396 min) | | | | |
| Compound 319/320 | A | A | A | B |
| (RT = 4.047 min) | | | | |
| 321 | B | B | | |
| 322 | B | A | B | A |
| 323 | C | B | | |
| 324 | A | A | A | A |
| 325 | A | A | A | A |
| 326 | A | A | A | A |
| 327 | B | A | A | B |
| 328 | A | B | A | A |
| 329 | A | B | C | A |
| 330 | A | A | B | A |
| 331 | A | A | A | A |
| 332 | B | A | A | A |
| 333 | B | B | | |
| 334 | B | A | A | A |
| 335 | B | A | A | A |
| 336 | A | A | A | A |
| 337 | A | A | A | A |
| 338 | A | A | A | A |
| 339 | B | A | B | A |
| 340 | A | A | A | A |
| 341 | A | A | A | A |
| 342 | A | A | A | A |
| 343 | A | A | A | A |
| 344 | B | A | A | A |
| Compound 345/346 | C | C | | |
| (RT = 1.286 min) | | | | |
| Compound 345/346 | A | A | A | A |
| (RT = 2.358 min) | | | | |
| 347 | B | A | B | A |
| 348 | B | C | | |
| 349 | B | A | C | A |
| 350 | A | A | A | A |
| 351 | A | A | A | A |
| 352 | A | A | A | A |
| 353 | A | A | A | A |
| 354 | A | A | A | A |
| 355 | C | A | | |
| 356 | A | A | A | A |
| 357 | B | A | A | A |
| 358 | B | A | | |
| 359 | A | B | B | A |
| 360 | A | B | A | A |
| 361 | A | B | A | A |
| 362 | A | B | A | A |
| 363 | B | B | | |
| 364 | A | B | A | A |
| 365 | B | B | | |
| 366 | B | C | | |
| 367 | C | C | | |
| Compound 368/369 | C | C | | |
| (RT = 2.235 min) | | | | |
| Compound 368/369 | A | B | A | A |
| (RT = 3.334 min) | | | | |
| 370 | A | B | A | A |
| 371 | A | B | A | A |
| Compound 372/373 | A | A | A | A |
| (RT = 4.48 min) | | | | |
| Compound 372/373 | A | A | A | A |
| (RT = 4.56 min) | | | | |
| 374 | B | B | | |
| 375 | A | A | A | A |
| Compound 376/377 | B | C | | |
| (RT = 4.40 min) | | | | |
| Compound 376/377 | B | B | A | A |
| (RT = 4.54 min) | | | | |
| Compound 378/379 | C | B | | |
| (RT = 3.572 min) | | | | |
| Compound 378/379 | A | B | A | A |
| (RT = 5.826 min) | | | | |
| 380 | A | B | A | A |
| 381 | A | B | A | A |
| 382 | B | B | B | A |
| 383 | A | B | | |
| 384 | A | B | A | A |
| 385 | B | B | C | A |
| 386 | B | B | | |
| 387 | C | C | | |
| 388 | A | B | B | A |
| Compound 389/390 | A | B | B | A |
| (RT = 2.32 min) | | | | |
| Compound 389/390 | A | B | A | A |
| (RT = 3.95 min) | | | | |
| Compound 391/392 | A | B | A | A |
| (RT = 3.06 min) | | | | |
| Compound 391/392 | A | B | B | A |
| (RT = 4.48 min) | | | | |
| 393 | A | B | A | A |
| 394 | A | B | B | A |
| 395 | B | C | B | A |
| 396 | B | B | | |
| 397 | A | B | A | A |
| 398 | A | A | B | A |
| 399 | A | B | A | A |
| 400 | B | B | B | A |
| Mixture of two | B | C | | |
| isomers of 401-1/401- | | | | |
| 2/402-1/402-2 (RT = | | | | |
| 5.504 min) | | | | |
| Mixture of the other | A | A | A | A |
| two isomers of 401- | | | | |
| 1/401-2/402-1/402-2 | | | | |
| (RT = 7.695 min) | | | | |
| 403 | C | C | | |
| 404 | A | B | A | A |
| 405 | B | C | C | B |
| 406 | A | B | A | A |
| Compound 407/408 | B | C | | |
| (RT = 2.628 min) | | | | |
| Compound 407/408 | A | B | A | A |
| (RT = 3.760 min) | | | | |
| 409 | A | B | A | A |
| 410 | A | B | B | A |
| 411 | A | B | A | A |
| 412 | A | B | C | B |
| 413 | A | B | B | A |
| 414 | B | A | A | A |
| 415 | B | B | A | B |
| 416 | A | B | A | A |
| 417 | A | B | A | A |
| 418 | A | B | A | A |
| 419 | A | A | A | A |
| 420 | A | B | A | A |
| 421 | A | B | A | A |
| 422 | C | B | C | B |
| 423 | A | B | A | A |
| 424 | C | C | | |
| 425 | A | B | A | A |
| Compound 426/427 | C | C | | |
| (RT = 2.312 min) | | | | |
| Compound 426/427 | A | B | A | A |
| (RT = 3.262 min) | | | | |
| 428 | B | B | A | A |
| 429 | A | C | | |
| 430 | A | B | A | A |
| 431 | A | A | A | A |
| 432 | C | B | | |
| 433 | B | B | | |
| 434 | B | B | | |
| 435 | A | A | A | A |
| 436 | A | A | A | A |
| 437 | A | A | C | A |
| 438 | A | B | B | A |
| Compound 439/440 | B | C | | |
| (439-1/440-1: RT = | | | | |
| 0.928 min as a starting | | | | |
| material) | | | | |
| Compound 439/440 | B | C | | |
| (439-1/440-1: RT = | | | | |
| 0.960 min as a starting | | | | |
| material) | | | | |
| Compound 441/442 | A | B | A | A |
| (RT = 4.47 min) | | | | |
| Compound 441/442 | B | B | | |
| (RT = 4.63 min) | | | | |
| 443 | B | C | | |
| 444 | B | A | B | A |
| 445 | C | B | | |
| 446 | A | B | A | A |
| 447 | C | B | | |
| 448 | A | A | B | A |
| 449 | B | B | A | A |
| 450 | B | A | A | A |
| 451 | A | B | A | A |
| 452 | B | C | | |
| 453 | A | B | A | A |
| 454 | C | C | | |
| 455 | C | C | | |
| 456 | C | C | | |
| 457 | C | C | | |
| 458 | C | B | | |
| 459 | B | A | A | A |
| 460 | A | B | A | A |
| 461 | B | B | B | A |
| 462 | B | C | C | A |
| 463 | B | B | A | A |
| 464 | B | B | B | A |
| 465 | B | B | A | A |
| 466 | C | C | | |
| 467 | A | B | A | A |
| 468 | B | B | B | A |
| 469 | B | B | A | A |
| 470 | C | B | B | A |
| 471 | B | B | A | A |
| 472 | C | C | | |
| 473 | B | B | B | A |
| 474 | B | B | A | A |
| 475 | B | C | B | B |
| 476 | C | B | C | A |
| 477 | A | B | A | A |
| 478 | B | B | B | A |
| 479 | B | B | A | A |
| 480 | A | B | A | A |
| 481 | B | B | B | A |
| 482 | A | B | A | A |
| 483 | B | B | A | B |
| 484 | B | B | A | B |
| 485 | B | B | A | A |
| 486 | B | B | A | A |
| 487 | B | B | A | A |
| 488 | B | B | A | A |
| 489 | B | B | B | A |
| 490 | B | B | B | A |
| 491 | B | B | A | A |
| 492 | A | B | A | A |
| 493 | B | B | B | A |
| 494 | C | C | | |
| 495 | B | B | A | A |
| 496 | A | B | | |
| 497 | A | B | | |
| 498 | A | B | | |
| 499 | A | B | | |
| 500 | B | C | | |
| 501 | B | C | | |
| 502 | C | C | | |
| 503 | C | C | | |
| 504 | B | B | | |
| 505 | C | C | | |
| 506 | A | B | | |
| 507 | C | B | | |
| 508 | A | B | B | A |
| 509 | A | A | B | A |
| 510 | A | A | A | A |
| 511 | B | A | A | B |
| 512 | B | B | B | A |
| 513 | B | B | B | A |
| 514 | B | A | B | A |
| 515 | C | B | B | A |
| 516 | A | B | A | A |
| 517 | B | B | A | B |
| 518 | B | B | A | B |
| 519 | B | B | B | A |
| 520 | C | C | | |
| 521 | A | B | B | A |
| 522 | B | B | B | A |
| 523 | A | C | B | A |
| 524 | C | C | B | A |
| 525 | C | C | | |
| 526 | B | B | B | A |
| 527 | A | B | A | A |
| 528 | C | C | | |
| 529 | B | C | | |
| Compound 530/531 | B | A | A | A |
| (RT = 2.900 min) | | | | |
| Compound 530/531 | A | B | A | B |
| (RT = 2.842 min) | | | | |
| 532 | C | C | | |
| Compound 533/534 | D | C | | |
| (RT = 1.313 min) | | | | |
| Compound 533/534 | A | B | A | B |
| (RT = 1.708 min) | | | | |
| 535 | B | A | A | A |
| Compound 536/537 | C | C | | |
| (RT = 2.013 min) | | | | |
| Compound 536/537 | A | A | A | A |
| (RT = 2.643 min) | | | | |
| 538 | C | C | B | A |
| 539 | C | B | B | B |
| 540 | A | B | A | A |
| 541 | B | B | A | A |
| 542 | C | B | B | B |
| 543 | B | B | B | A |
| 544 | B | C | B | B |
| Compound 545/546 | D | C | | |
| (RT = 6.524 min) | | | | |
| Compound 545/546 | A | A | A | A |
| (RT = 9.713 min) | | | | |
| 547 | B | A | A | A |
| 548 | B | B | A | A |
| Compound 549/550 | B | A | A | A |
| (RT = 3.877 min) | | | | |
| Compound 549/550 | C | C | | |
| (RT = 4.624 min) | | | | |
| 551 | B | B | A | A |
| 552 | A | A | A | A |
| Compound 553/554 | C | C | | |
| (RT = 3.614 min) | | | | |
| Compound 553/554 | B | A | A | B |
| (RT = 4.617 min) | | | | |
| 555 | C | B | A | A |
| 556 | C | B | | |
| 557 | B | A | A | A |
| Compound 558/559 | B | B | | |
| (RT = 2.41 min) | | | | |
| Compound 558/559 | C | A | B | B |
| (RT = 2.50 min) | | | | |
| 560 | C | B | | |
| 561 | C | C | | |
| 562 | B | B | A | B |
| 563 | B | A | A | A |
| 564 | C | C | | |
| 565 | A | A | A | A |
| 566 | B | A | A | A |
| 567 | A | B | A | A |
| 568 | A | B | A | B |
| Compound 569/644 | C | B | B | B |
| (RT = 9.799 min) | | | | |
| Compound 570/571 | C | C | | |
| (RT = 5.999 min) | | | | |
| Compound 570/571 | A | A | A | A |
| (RT = 7.223 min) | | | | |
| 572 | C | C | | |
| 573 | C | C | C | C |
| 574 | B | B | B | A |
| 575 | B | C | | |
| Compound 576-1/576- | B | B | A | A |
| 2/577 (RT = 1.11 min) | | | | |
| Compound 576-1/576- | C | C | | |
| 2/577 (RT = 1.19 min) | | | | |
| 578 | C | B | B | B |
| Compound 579/645 | C | B | B | B |
| (RT = 1.11 min) | | | | |
| 580 | B | A | B | B |
| 581 | B | A | B | B |
| 582 | C | C | C | B |
| 583 | B | A | A | A |
| 584 | B | B | | |
| 585 | B | B | B | A |
| 586 | C | C | | |
| 587 | C | C | | |
| 588 | C | B | B | A |
| 589 | B | A | A | A |
| 590 | B | B | B | B |
| 591 | C | B | B | B |
| 592 | A | A | A | A |
| 593 | C | B | B | B |
| 594 | C | B | A | B |
| 595 | B | B | B | A |
| 596 | B | B | B | A |
| 597 | B | B | A | A |
| 598 | B | B | B | A |
| 599 | C | C | B | B |
| 600 | C | C | C | C |
| 601 | B | A | A | B |
| 602 | C | B | B | A |
| 603 | C | C | B | B |
| 604 | C | C | C | C |
| 605 | B | A | A | A |
| 606 | B | A | A | A |
| 607 | C | C | C | B |
| 608 | A | A | B | A |
| 609 | B | B | B | A |
| 610 | C | B | B | B |
| 611 | C | C | C | B |
| 613 | A | A | B | B |
| 614 | B | B | C | C |
| 615 | C | B | B | B |
| 616 | B | A | A | B |
| Compound 617/618 | D | C | | |
| (RT = 2.253 min) | | | | |
| Compound 617/618 | B | A | A | B |
| (RT = 2.932 min) | | | | |
| 619 | C | A | B | A |
| 620 | C | B | B | B |
| 621 | C | B | B | A |
| 622 | C | A | B | A |
| 623 | C | A | B | A |
| 624 | B | B | B | B |
| 625 | B | A | B | A |
| 626 | C | B | B | A |
| 627 | C | B | B | B |
| 628 | C | B | A | B |
| 629 | A | B | A | B |
| 630 | C | C | | |
| 631 | C | A | B | A |
| 632 | C | B | B | A |
| Compound 633/634 | C | C | | |
| (RT = 2.036 min) | | | | |
| Compound 633/634 | B | A | A | B |
| (RT = 2.496 min) | | | | |
| 635 | B | B | B | A |
| 636 | C | C | | |
| 637 | C | B | A | B |
| 638 | B | A | A | B |
| 639 | C | B | B | B |
| 640 | C | A | B | B |
| 641 | C | A | A | B |
| 642 | C | B | | |
| 643 | B | B | | |
| Compound 569/644 | D | C | | |
| (RT = 8.031 min) | | | | |
| Compound 579/645 | D | C | | |
| (RT = 1.19 min) | | | | |

As shown in Table 2, the term “Compound X/Y (RT=Z min)” means the isomer of Compounds X or Y that has a retention time (RT) of Z min. For example, “Compound 50/51 (RT=5.19 min)” means the isomer of Compound 50 or Compound 51 that has a retention time of 5.19 min. “Compound 50/51 (RT=5.41 min)” means the isomer of Compound 50 or Compound 51 that has a retention time of 5.41 min.

All publications, including but not limited to disclosures and disclosure applications, cited in this specification are herein incorporated by reference as though fully set forth. If certain content of a publication cited herein contradicts or is inconsistent with the present disclosure, the present disclosure controls.

One skilled in the art will readily recognize from the disclosure and claims that various changes, modifications, and variations can be made therein without departing from the spirit and scope of the disclosure as defined in the following claims.

1. A compound of the following structural Formula 1: 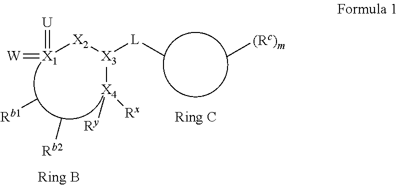
a tautomer thereof, a solvate or stereoisomer of the compound or the tautomer, or a pharmaceutically acceptable salt of the foregoing, wherein:
X₁ is C or S, provided that when X₁ is C, W is absent, and when X₁ is S, W is O or absent;
U is selected from O and S provided that when X₁ is S, U cannot be S;
X₂ is C, N, or absent; X₃ is C or N; X₄ is C, N, or absent;
Ring B is a 5- to 6-membered heterocyclic group, wherein Ring B comprises zero or one heteroatom at a position other than the positions of X₁, X₂, X₃, and X₄;
Ring C is phenyl, a 9- to 10-membered aryl, a 5- to 6-membered heteroaryl, a 9-to 10-membered heteroaryl, a 3- to 6-membered carbocyclyl, or a 4- to 12-membered heterocyclic group;
L is selected from 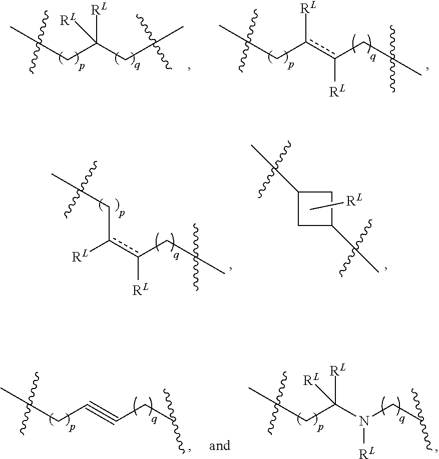
wherein R^{L}, for each occurrence, is independently selected from H, deuterium, halogen, and C₁ to C₃ alkyl optionally substituted with 1-3 groups selected from halogen;
R^{b1} and R^{b2} are attached to two adjacent positions in Ring B and R^{b1} and R^{b2} join to form Ring A, wherein Ring A is a phenyl or 5- to 6-membered heteroaryl group and wherein Ring A is substituted with z groups of R^{a};
R^{a}, for each occurrence, is independently selected from halogen, CN, C₁ to C₄ alkyl, —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3- to 7-membered carbocyclyl, and 3- to 7-membered heterocyclyl,
wherein:
the C₁ to C₄ alkyl of R^{a} is optionally substituted with 1-3 groups selected from —NR^{p}R^{q}, —C(═O)NR^{p}R^{q}, phenyl, 5- to 6-membered heteroaryl, 3- to 7-membered carbocyclyl, 3- to 7-membered heterocyclyl, halogen, and OR^{s},
the 5- to 6-membered heteroaryl, the 3- to 7-membered carbocyclyl, or the 3- to 7-membered heterocyclyl of R^{a} is optionally substituted with 1-2 groups selected from C₁ to C₄ alkyl, —O(C₁ to C₄ alkyl), —N(C₁ to C₄ alkyl)₂, and —NH(C₁ to C₄ alkyl);
R^{x} is selected from H, ═O, CN, C₃ to C₆ carbocyclyl, C₁ to C₄ alkyl, 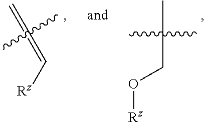
wherein:
the C₁ to C₄ alkyl of R^{x} is optionally substituted with 1-3 groups selected from halogen, deuterium, CN, 5- to 6-membered carbocyclyl, 5- to 9-membered heterocyclyl, 6-membered aryl, 5- to 6-membered heteroaryl, and OH,
wherein: the 6-membered aryl or the 5- to 6-membered heteroaryl of the C₁ to C₄ alkyl of R^{x} is optionally substituted with 1-3 groups selected from halogen, OR^{s}, CN, C(═O)NR^{p}R^{q}, NR^{p}R^{q}, C₁-C₆ alkyl (which is optionally substituted with 1-3 groups selected from halogen, O—R^{z}, OR^{s}, and 4- to 6-membered heterocyclyl optionally substituted with C₁ to C₂ alkyl), C₁-C₆ alkenyl, C₁-C₆ alkynyl, 3- to 6-membered carbocyclyl, and 4- to 7-membered heterocyclyl (which is optionally substituted with 1-3 groups selected from optionally substituted C₁ to C₄ alkyl, halogen, and OR^{s}), and
wherein: the 5- to 9-membered heterocyclyl of the C₁ to C₄ alkyl of R^{x} is optionally substituted with 1-2 groups selected from C₁ to C₆ alkyl, ═O, and halogen,
R^{z} is selected from H, 6-membered aryl, and 5- to 6-membered heteroaryl,
wherein: the 6-membered aryl or 5- to 6-membered heteroaryl of R^{z} is optionally substituted with 1-2 groups selected from halogen;
R^{y} is selected from H, C₁ to C₂ alkyl, and absent;
R^{c}, for each occurrence, is independently selected from deuterium, halogen, C₁ to C₄ alkyl, ═O, ═S, OR^{s}, —NHC(═O)R^{s}, —NHC(═O)OR^{s}, NR^{p}R^{q}, —NHC(═O)NR^{p}R^{q}, CN, optionally substituted 5- to 6-membered heteroaryl, optionally substituted 5- to 6-membered heterocyclyl, and —C(═O)NR^{p}R^{q},
wherein:
the C₁ to C₄ alkyl of R^{c} is optionally substituted with 1-3 groups selected from halogen and OR^{s};
wherein:
R^{p}, for each occurrence, is independently selected from H and C₁ to C₆ alkyl, R^{q}, for each occurrence, is independently selected from H, C₁ to C₆ alkyl, phenyl, 5- to 6-membered heteroaryl, 4- to 7-membered heterocyclyl, and CN, or
R^{p} and R^{q} join to form a 3- to 6-membered carbocyclyl;
R^{s}, for each occurrence, is independently selected from H, C₁ to C₆ alkyl (which is optionally substituted with 1-3 groups selected from halogen, deuterium, O(C₁ to C₄ alkyl), and NR^{p}R^{q}), phenyl, and 4- to 7-membered heterocyclyl;
wherein:
the 4- to 7-membered heterocyclyl of R^{q} and R^{s}, in each occurrence, is independently optionally substituted with 1-2 groups selected from C₁ to C₄ alkyl and —O(C₁ to C₄ alkyl); and
wherein:
m is an integer selected from 0, 1, 2, and 3;
p is an integer selected from 0, 1, 2, and 3;
q is an integer selected from 0, 1, 2, and 3;
z is an integer selected from 0, 1, 2, 3, and 4; and
the sum of p and q is an integer equal to or less than 5.

2. The compound of claim 1, wherein the compound has the following structural Formula 2a or Formula 2a′: 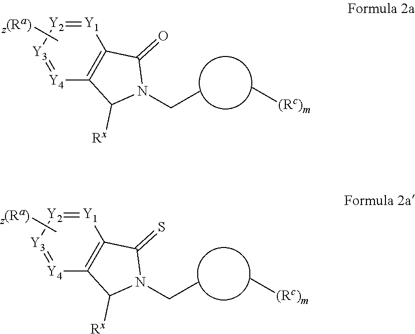
a tautomer thereof, a solvate or stereoisomer of the compound or the tautomer, or a pharmaceutically acceptable salt of the foregoing, wherein Y₁, Y₂, Y₃, and Y₄ are independently selected from C and N.

3. The compound of claim 1, wherein the compound has the following structural Formula 2b: 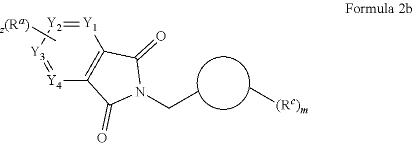
a tautomer thereof, a solvate or stereoisomer of the compound or the tautomer, or a pharmaceutically acceptable salt of the foregoing, wherein Y₁, Y₂, Y₃, and Y₄ are independently selected from C and N.

4. The compound of claim 1, wherein the compound has the following structural Formula 2c, 2c-1, or 2c-2: 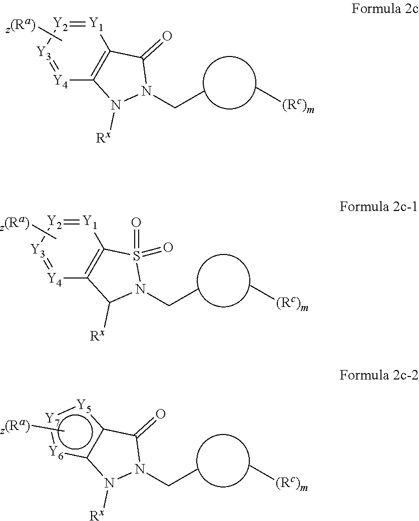
a tautomer thereof, a solvate or stereoisomer of the compound or the tautomer, or a pharmaceutically acceptable salt of the foregoing, wherein Y₁, Y₂, Y₃ and Y₄ are independently selected from C and N, and Y₅, Y₆, and Y₇ are independently selected from N, S, O and C.

5. The compound of claim 1, wherein the compound has a structural formula selected from Formulae 2d to 2i: 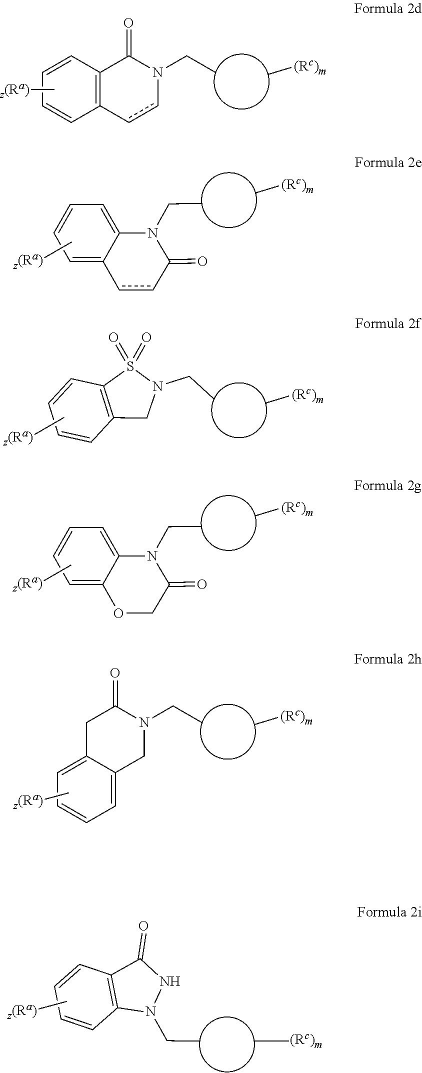
a tautomer thereof, a solvate or stereoisomer of the compound or the tautomer, or a pharmaceutically acceptable salt of the foregoing.

6. The compound of claim 1, wherein the compound has a structural formula selected from Formulae 3a and 3a-1 to 3a-16: 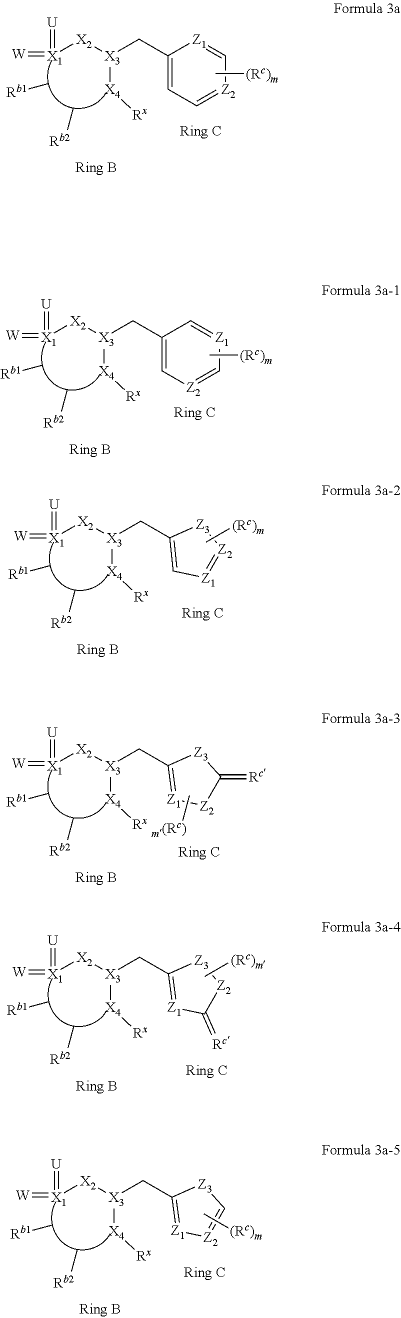 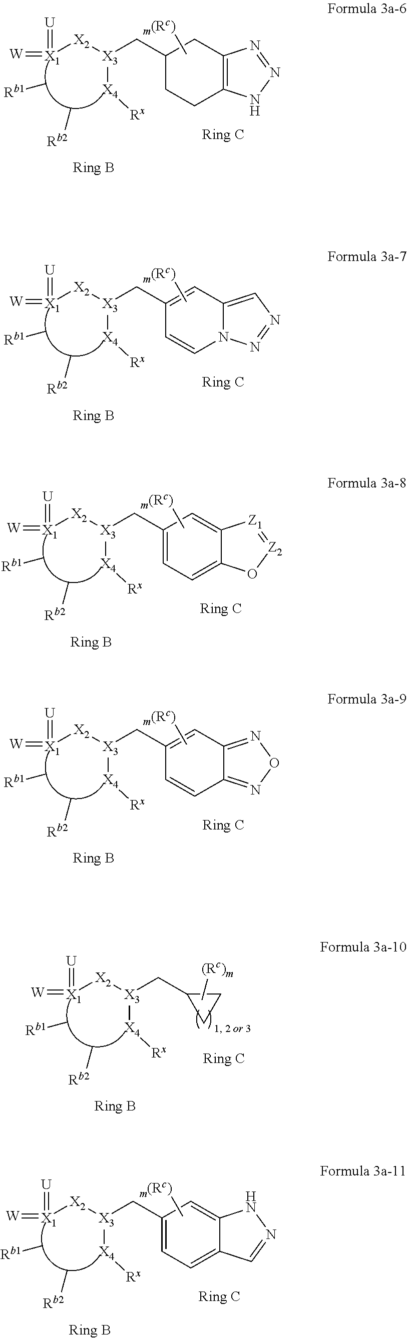 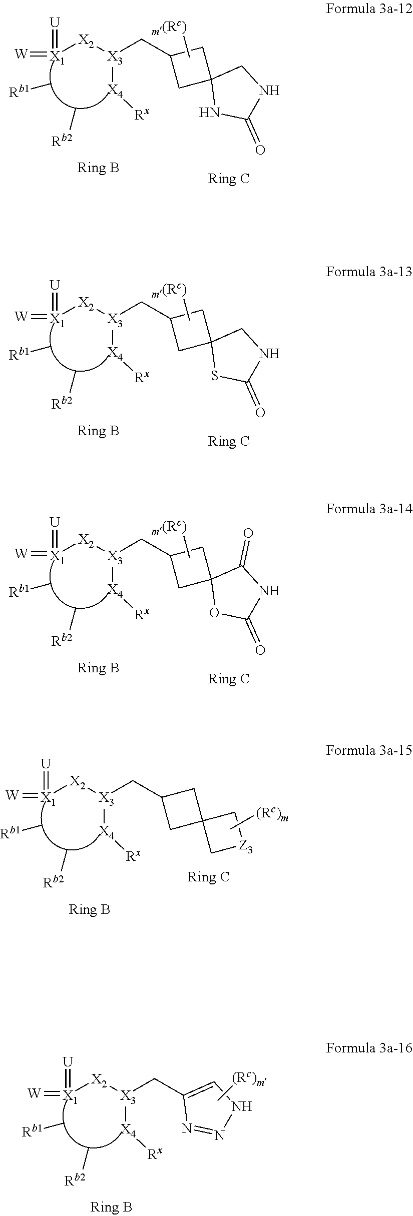
a tautomer thereof, a solvate or stereoisomer of the compound or the tautomer, or a pharmaceutically acceptable salt of the foregoing, wherein Z₁ and Z₂ are independently selected from C and N, Z₃ is selected from C, N, S, and O, R^{c′} is O or S, and m′, for each occurrence, is independently selected from 0, 1, and 2.

7. The compound of claim 1, wherein the compound has a structural formula selected from Formulae 3b-3c: 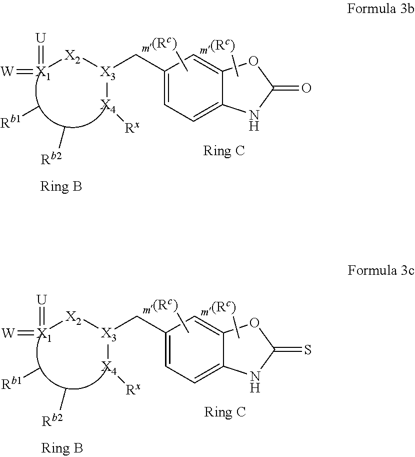
a tautomer thereof, a solvate or stereoisomer of the compound or the tautomer, or a pharmaceutically acceptable salt of the foregoing, wherein m′ is selected from 0 and 1.

8. The compound of claim 1, wherein the compound has a structural formula selected from Formulae 3d-3e: 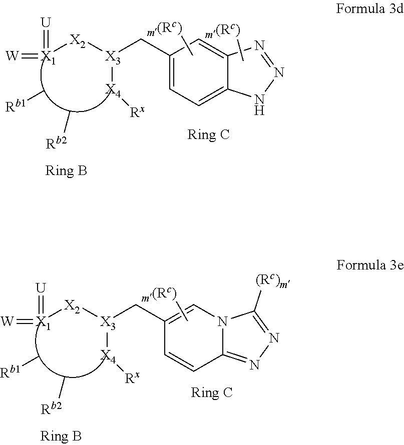
a tautomer thereof, a solvate or stereoisomer of the compound or the tautomer, or a pharmaceutically acceptable salt of the foregoing, wherein m′, for each occurrence, is independently selected from 0 and 1.

9. The compound of claim 1, wherein the compound has a structural formula selected from Formulae 3f-3h: 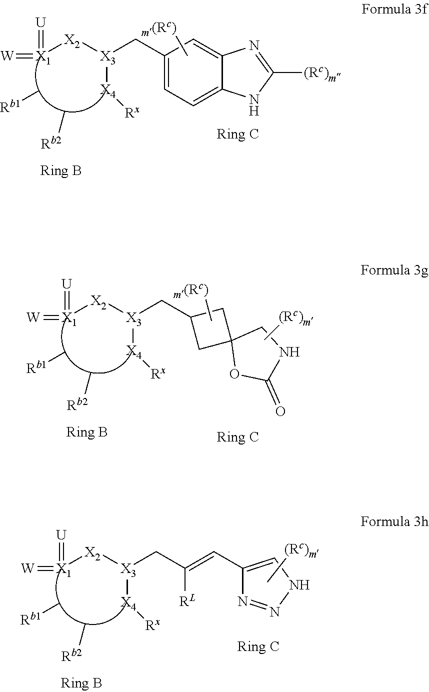
a tautomer thereof, a solvate or stereoisomer of the compound or the tautomer, or a pharmaceutically acceptable salt of the foregoing, wherein m′, for each occurrence, is independently selected from 0, 1, and 2, m″, for each occurrence, is independently selected from 0 and 1, and R^{L} is selected from H and F.

10. The compound of claim 1, wherein the compound has a structural formula selected from Formulae 4a-4h: 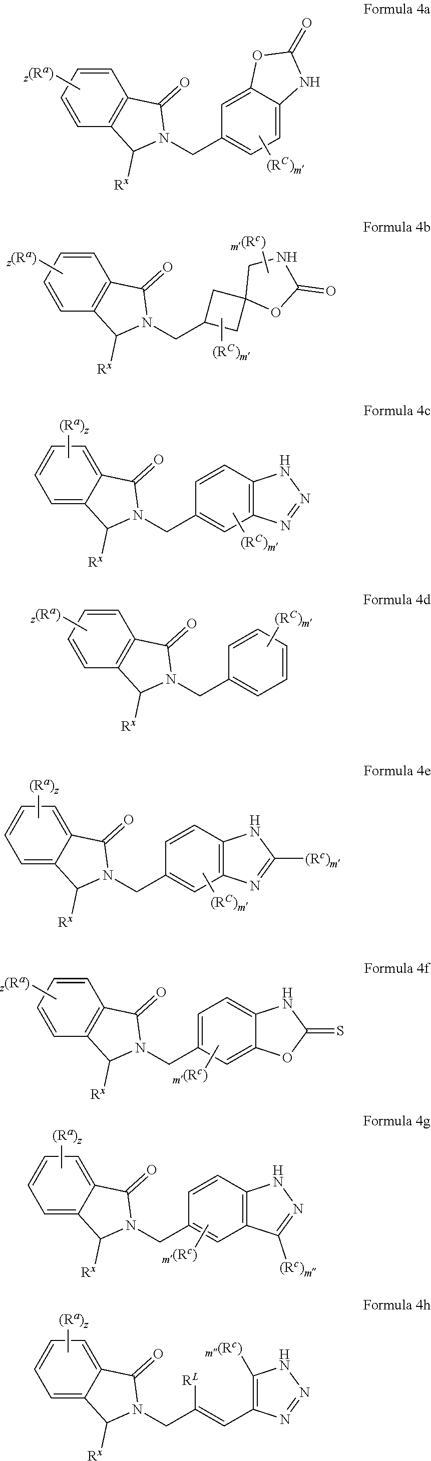
a tautomer thereof, a solvate or stereoisomer of the compound or the tautomer, or a pharmaceutically acceptable salt of the foregoing, wherein m′, for each occurrence, is independently selected from 0, 1 and 2, m″, for each occurrence, is independently selected from 0 and 1, and R^{L} is selected from H and F.

11. The compound of claim 1, wherein the compound has a structural formula selected from Formulae 5a-5e: 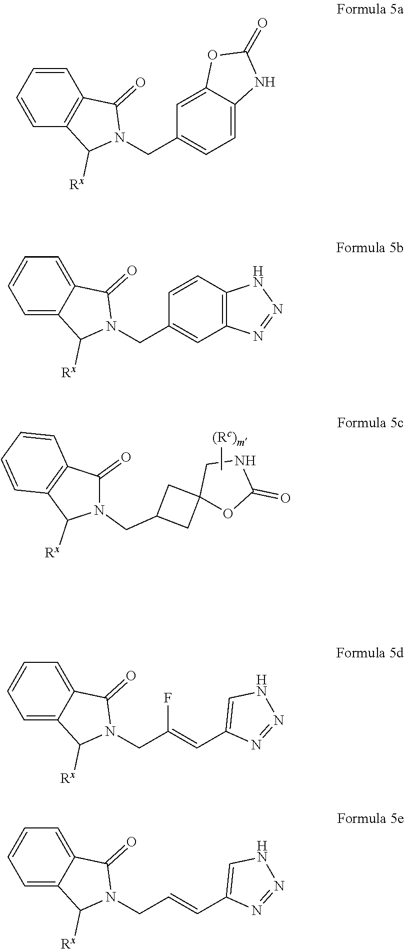
a tautomer thereof, a solvate or stereoisomer of the compound or the tautomer, or a pharmaceutically acceptable salt of the foregoing, R^{c} is deuterium, and m′ is selected from 0, 1, and 2.

12. The compound of claim 1, wherein the compound has a structural formula selected from Formulae 6a-6d: 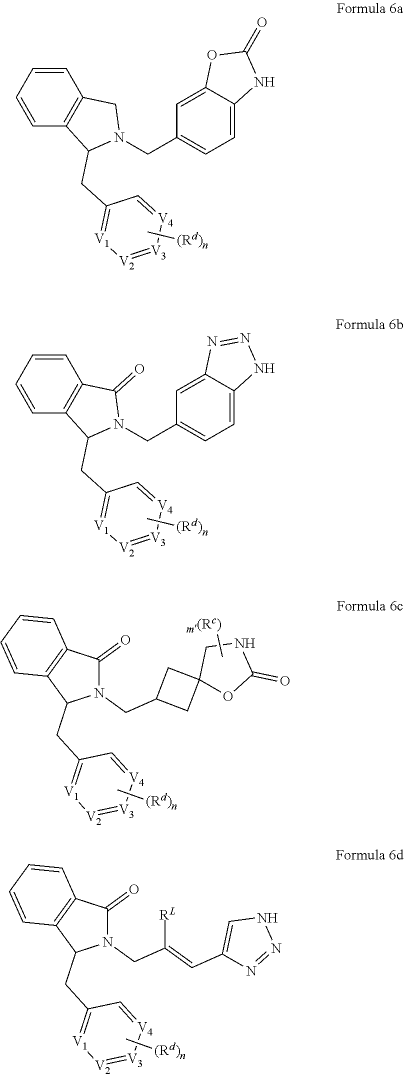
a tautomer thereof, a solvate or stereoisomer of the compound or the tautomer, or a pharmaceutically acceptable salt of the foregoing, wherein V₁, V₂, V₃, and V₄ are independently selected from C and N, R^{L} is selected from H and F, R^{c} is deuterium, R^{d}, for each occurrence, is independently selected from halogen, CN, OCH₃, C₁ to C₄ alkyl, C₁-C₄ alkenyl, C₁-C₄ alkynyl, 3- to 6-membered carbocyclyl, and 3- to 6-membered heterocyclyl, m′ is selected from 0, 1, and 2, and n is selected from 0, 1, 2, and 3.

13. The compound of claim 1, wherein the compound has a structural formula selected from Formulae 6e-6h: 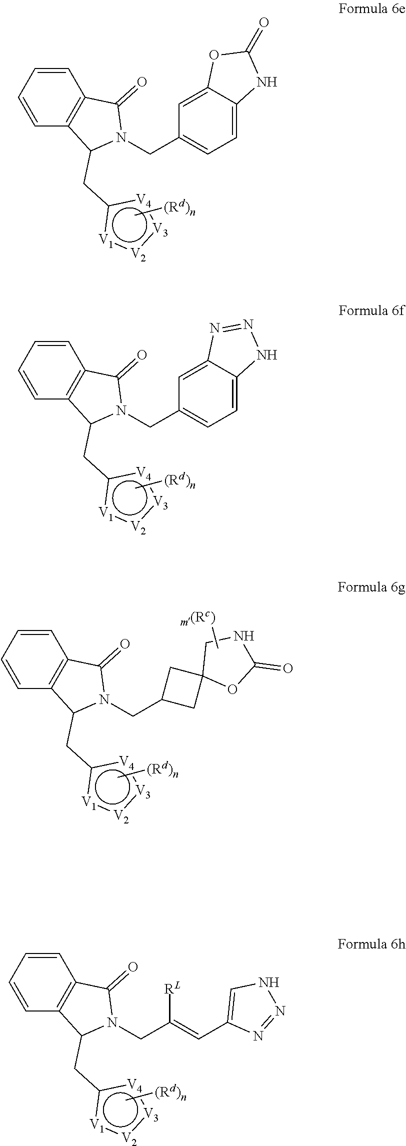
a tautomer thereof, a solvate or stereoisomer of the compound or the tautomer, or a pharmaceutically acceptable salt of the foregoing, wherein V₁, V₂, V₃, and V₄ are independently selected from C, O, S, and N, R^{L} is selected from H and F, R^{c} is deuterium, R^{d}, for each occurrence, is independently selected from halogen, CN, OCH₃, C₁ to C₄ alkyl, C₁-C₄ alkenyl, C₁-C₄ alkynyl, 3- to 6-membered carbocyclyl, and 3- to 6-membered heterocyclyl, m′ is selected from 0, 1, and 2, and n is selected from 0, 1, 2 and 3.

14. The compound, tautomer, solvate, stereoisomer, or pharmaceutically acceptable salt of claim 1, wherein Ring A is selected from phenyl, pyridyl, and 5-membered heteroaryl containing 1 to 2 heteroatoms selected from N, S, and O.

15. The compound, tautomer, solvate, stereoisomer, or pharmaceutically acceptable salt of claim 1, wherein X₁ is C, X₂ is absent, and X₃ is C or N.

16. The compound, tautomer, solvate, stereoisomer, or pharmaceutically acceptable salt of claim 1, wherein Ring C is selected from: 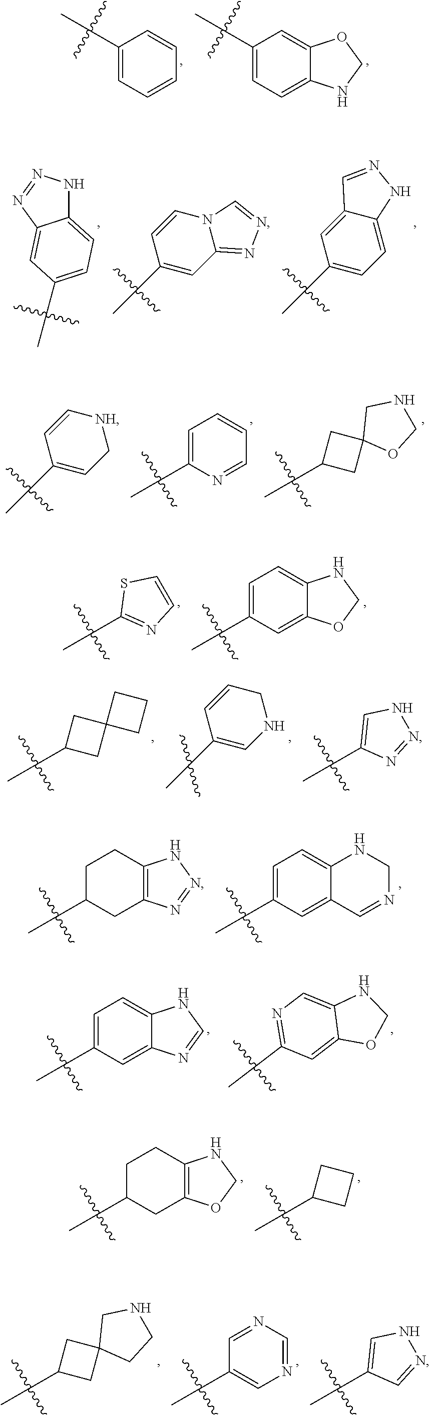 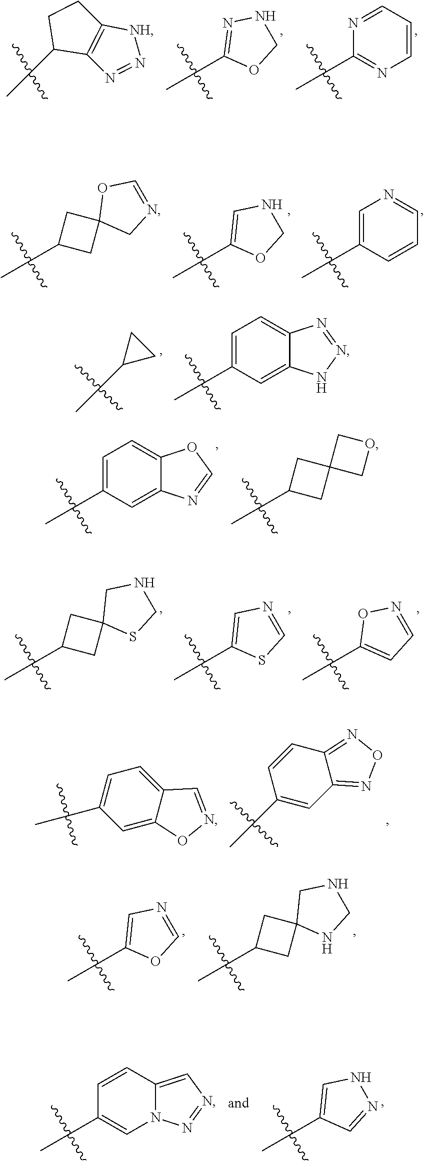
and wherein Ring C is substituted with m groups of R^{c}.

17. The compound, tautomer, solvate, stereoisomer, or pharmaceutically acceptable salt of claim 1, wherein R^{a} is selected from halogen, CN, C₁ to C₃ alkyl, —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3- to 7-membered carbocyclyl, and 3- to 7-membered heterocyclyl,
wherein:
the C₁ to C₃ alkyl of R^{a} is optionally substituted with 1-2 groups selected from —NR^{p}R^{q}, —C(═O)NR^{p}R^{q}, phenyl, 4- to 7-membered heterocyclyl, halogen, and OR^{s}, the 5- to 6-membered heteroaryl, 3- to 7-membered carbocyclyl, or 3- to 7-membered heterocyclyl of R^{a} is optionally substituted with one group selected from C₁ to C₃ alkyl, —O(C₁ to C₃ alkyl), —N(C₁ to C₃ alkyl)₂, and —NH(C₁ to C₃ alkyl), and
wherein:
R^{p}, for each occurrence, is independently selected from H and C₁ to C₃ alkyl,
R^{q}, for each occurrence, is independently selected from H, C₁ to C₃ alkyl, phenyl, 5- to 6-membered heteroaryl, and 4- to 7-membered heterocyclyl, and
R^{s}, for each occurrence, is independently selected from H, C₁ to C₃ alkyl (which is optionally substituted with 1-2 groups selected from halogen), phenyl, and 4- to 7-membered heterocyclyl,
wherein:
the 4- to 7-membered heterocyclyl of R^{q} and R^{s}, in each occurrence, is independently optionally substituted with one group selected from C₁ to C₃ alkyl and —O(C₁ to C₃ alkyl).

18. The compound, tautomer, solvate, stereoisomer, or pharmaceutically acceptable salt of claim 1, wherein R^{a} is selected from F, Br, Cl, CN, methyl, ethyl, —C(CH₃)₃, —CH(CH₃)₂, CH₂N(CH₃)₂, CH₂CH₂C(═O)NH₂, 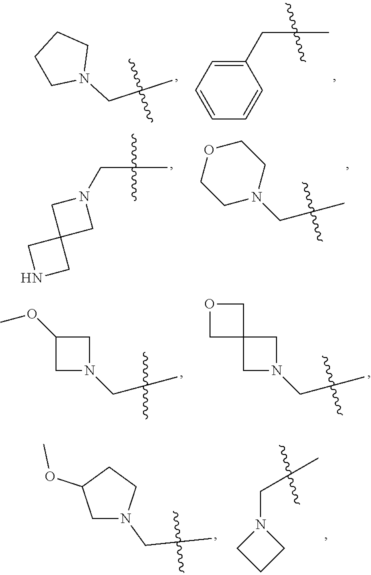
NH₂, —N(CH₃)₂, 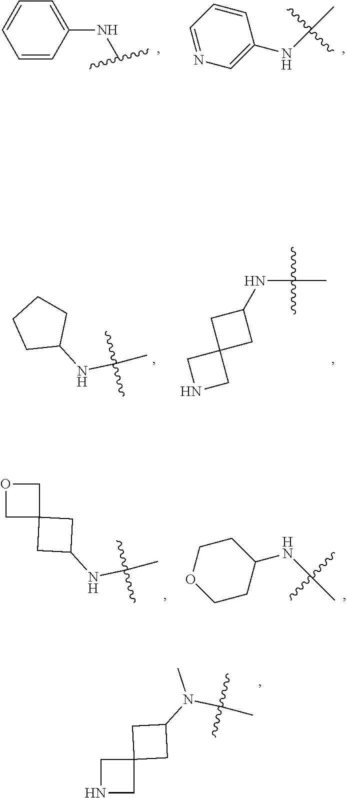
—NHC(═O)CH₃, —NHS(═O)₂CH₃, 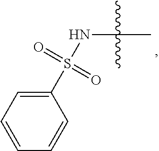 [SMILES: *NS(=O)(=O)c1ccccc1]
OH, OCH₃, 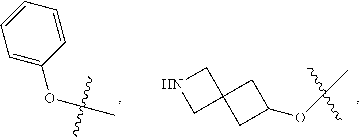 [SMILES: *Oc1ccccc1 ; *OC1CC2(CNC2)C1]
—C(═O)NH₂, —C(═O)NHCH₃, 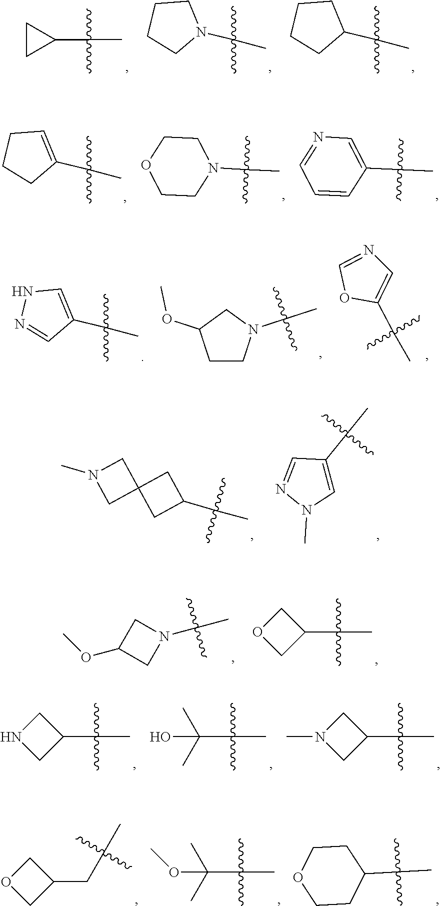
CH₂NHCH₃, CH₂OCH₃, —CHF₂, 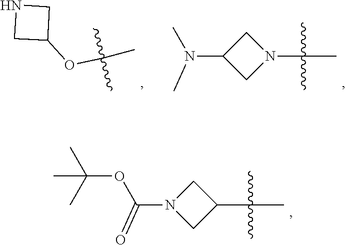
and OCHF₂.

19. The compound, tautomer, solvate, stereoisomer, or pharmaceutically acceptable salt of claim 1, wherein R^{a} is selected from F, Br, Cl, CN, OH, 5- to 6-membered heteroaryl, 3- to 6-membered carbocyclyl, 3- to 6-membered heterocyclyl, and methyl optionally substituted with 3- to 6-membered heterocyclyl.

20. The compound, tautomer, solvate, stereoisomer, or pharmaceutically acceptable salt of claim 1, wherein
R^{x} is selected from H, ═O, CN, 3-6 carbocyclyl, C₁ to C₂ alkyl, 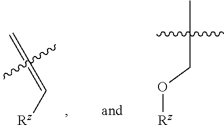
wherein:
the C₁ to C₂ alkyl of R^{x} is optionally substituted with 1-3 groups selected from halogen, deuterium, CN, 5- to 6-membered carbocyclyl, 5- to 9-membered heterocyclyl, 6-membered aryl, 5- to 6-membered heteroaryl, and OH,
wherein: the 6-membered aryl or the 5- to 6-membered heteroaryl of the C₁ to C₂ alkyl of R^{x} is optionally substituted with 1-3 groups selected from halogen, OR^{s}, CN, CONH₂, NH₂, C₁-C₄ alkyl (which is optionally substituted with 1-3 groups selected from halogen, O—R^{z}, OR^{s}, and 4- to 6-membered heterocyclyl optionally substituted with C₁ to C₂ alkyl), C₁-C₃ alkenyl, C₁-C₃ alkynyl, 3- to 4-membered carbocyclyl, and 4- to 7-membered heterocyclyl (which is optionally substituted with 1-3 groups selected from C₁ to C₂ alkyl, halogen, and OR^{s}) and
wherein: the 5- to 9-membered heterocyclyl of the C₁ to C₂ alkyl of R^{x} is optionally substituted with 1-2 groups selected from C₁ to C₂ alkyl, ═O, and halogen,
wherein:
R^{s} is selected from H and C₁ to C₂ alkyl optionally substituted with 1-3 groups selected from halogen, O(C₁ to C₂ alkyl), —N(C₁ to C₂ alkyl)(C₁ to C₂ alkyl), and
R^{z} is selected from H, 6-membered aryl, and 5- to 6-membered heteroaryl,
wherein: the 6-membered aryl or 5- to 6-membered heteroaryl of R^{z} is optionally substituted with 1-2 groups selected from halogen.

21. The compound, tautomer, solvate, stereoisomer, or pharmaceutically acceptable salt of claim 1, wherein R^{x} is selected from C₁ to C₂ alkyl, wherein the C₁ to C₂ alkyl of R^{x} is substituted with 6-membered aryl or 5- to 6-membered heteroaryl, wherein: the 6-membered aryl or the 5- to 6-membered heteroaryl of the C₁ to C₂ alkyl of R^{x} is optionally substituted with 1-3 groups selected from halogen, OCH₃, CN, CONH₂, NH₂, C₁-C₂ alkyl (which is optionally substituted with 1-3 groups selected from halogen and O—R^{z}), O(C₁ to C₂ alkyl) (which is optionally substituted with 1-3 groups selected from halogen), 3- to 4-membered carbocyclyl, and 4- to 7-membered heterocyclyl.

22. The compound, tautomer, solvate, stereoisomer, or pharmaceutically acceptable salt of claim 1, wherein R^{x} is selected from H, ═O, CN, methyl, ethyl, propyl, CH₂CN

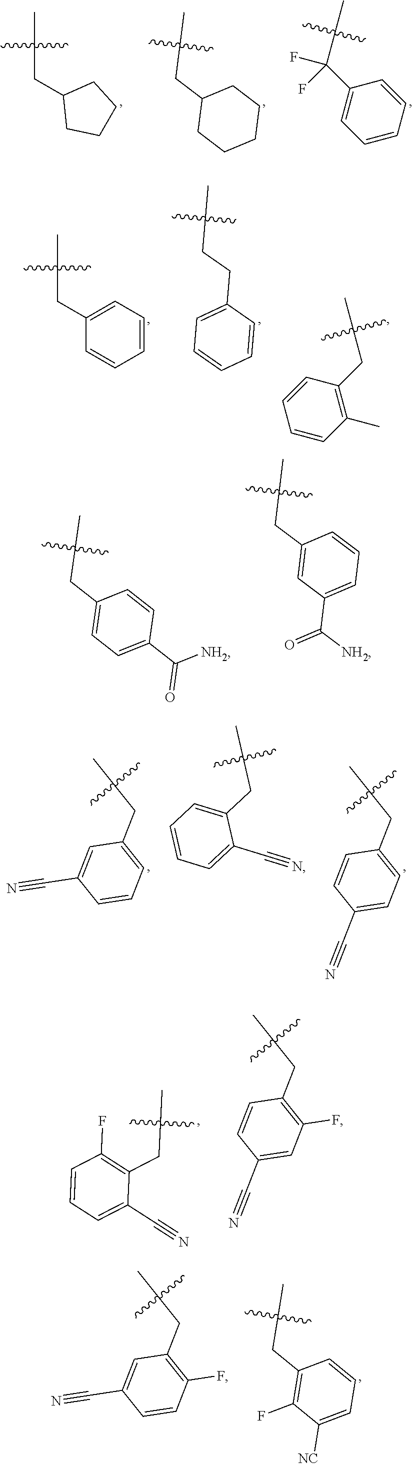

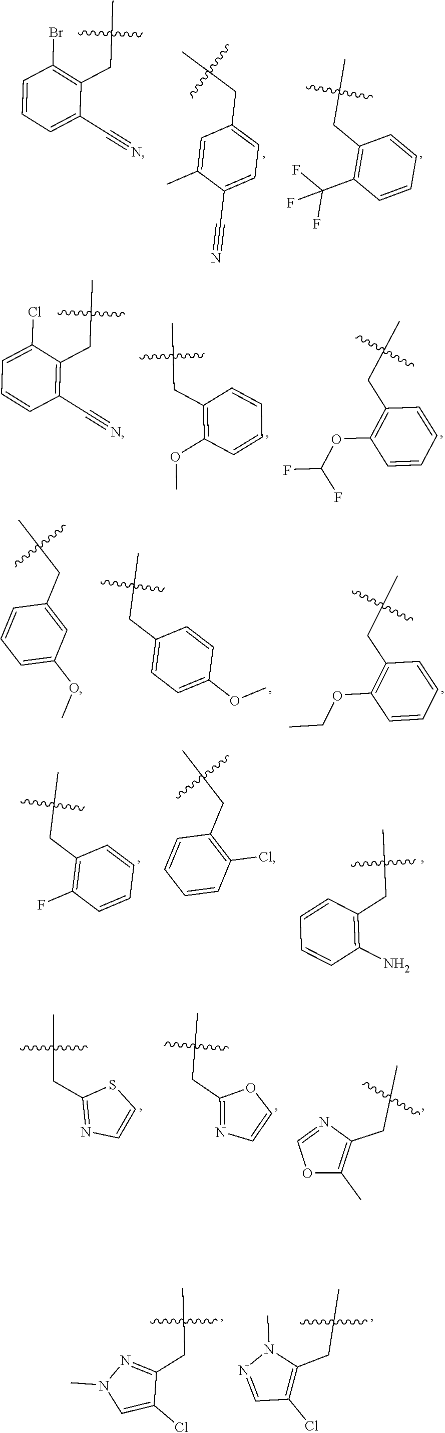

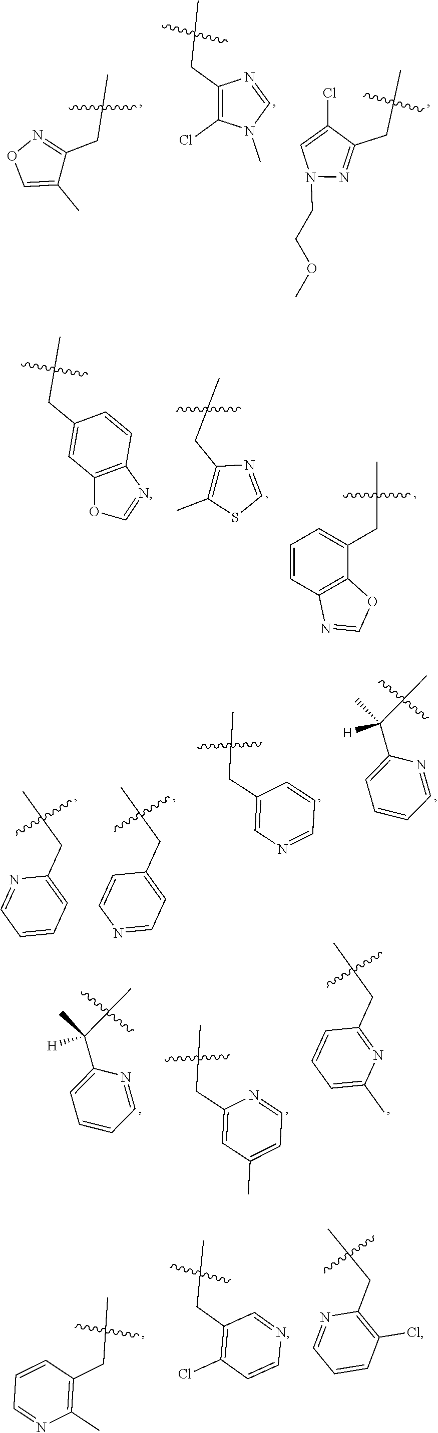

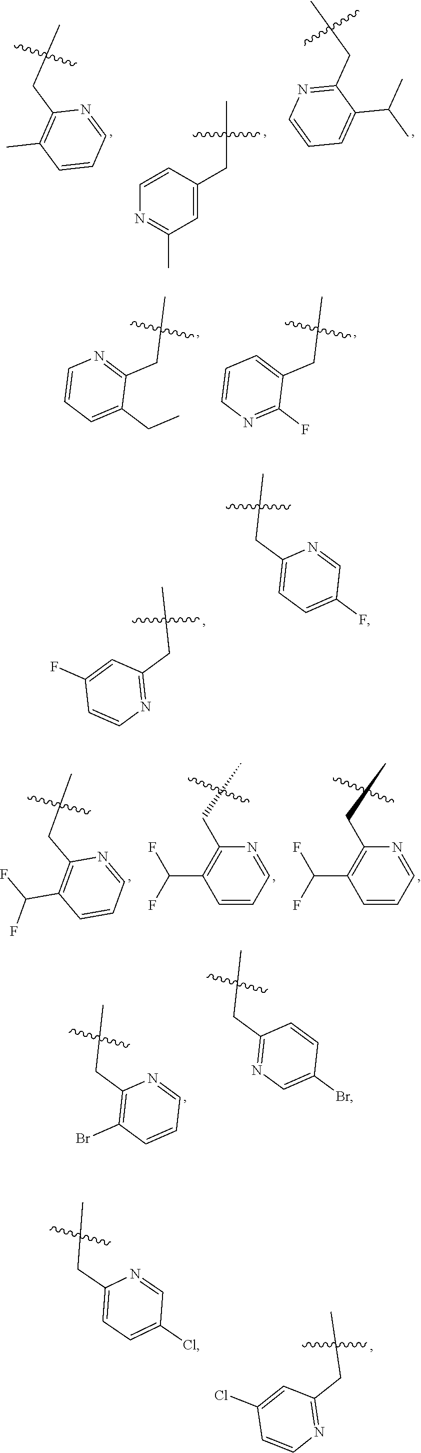

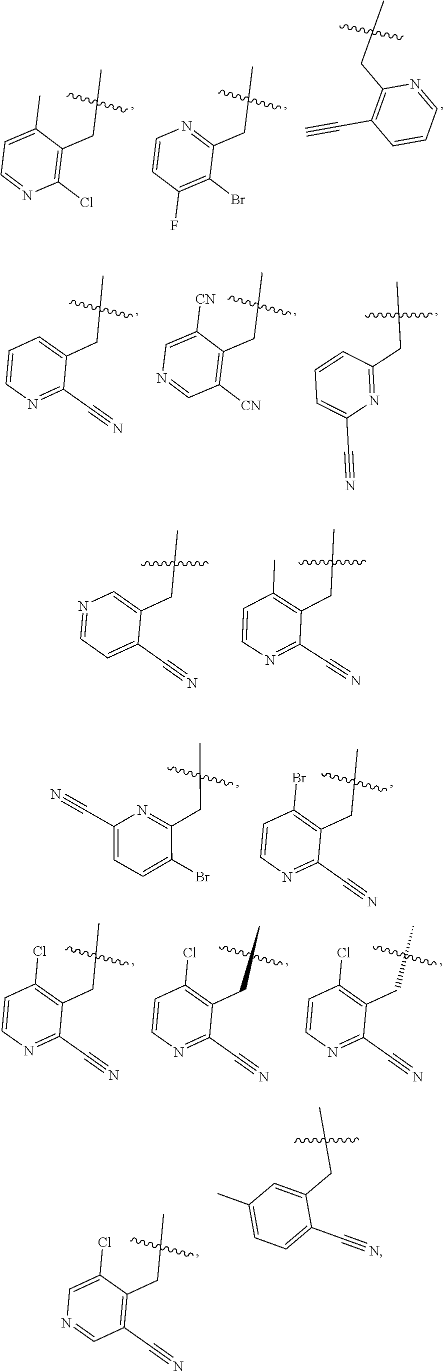

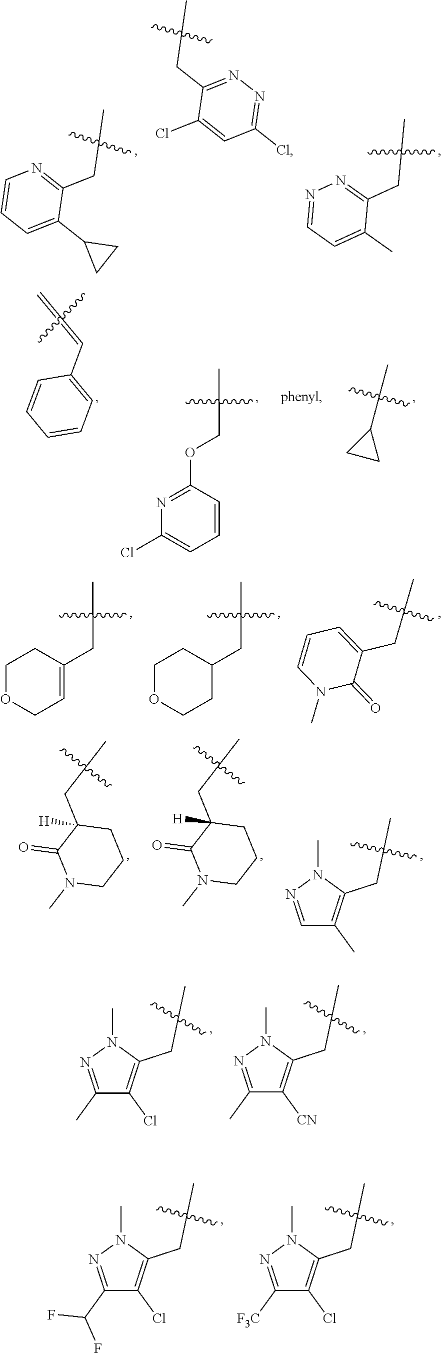

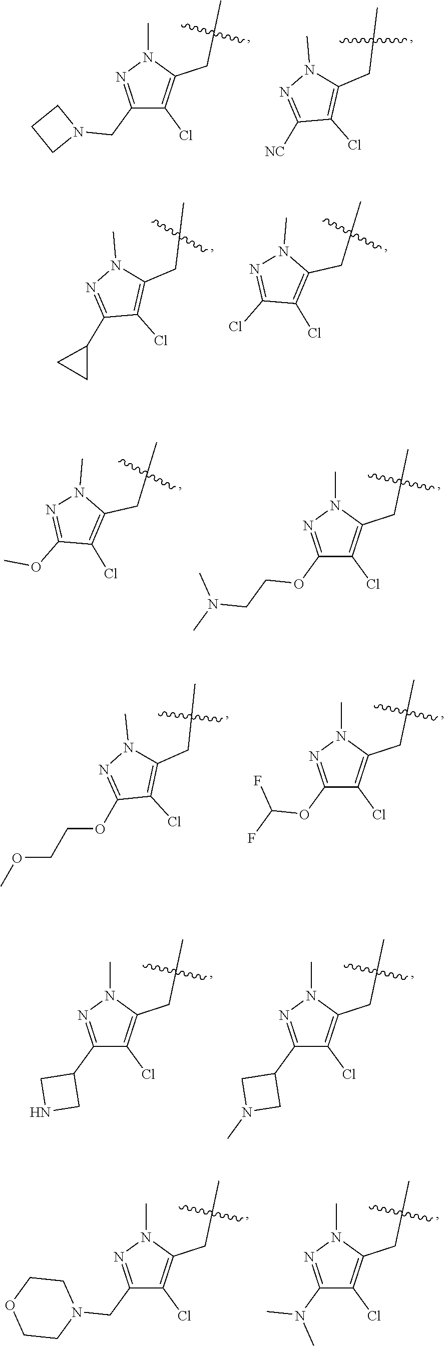

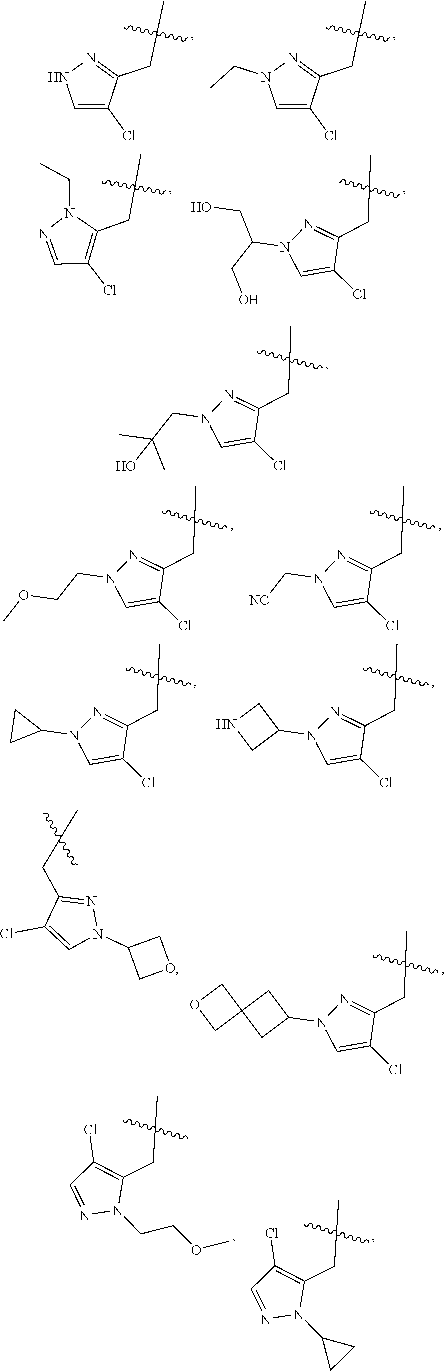

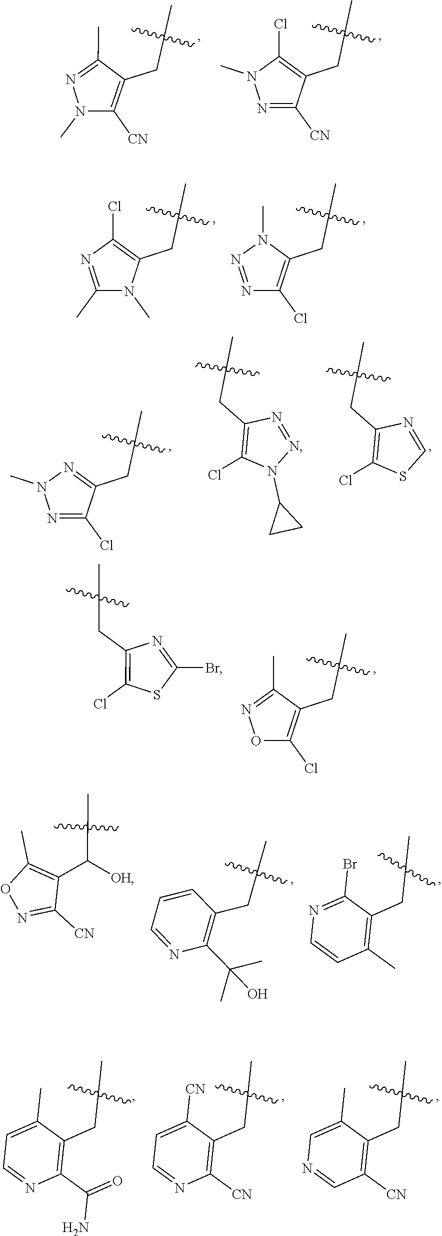

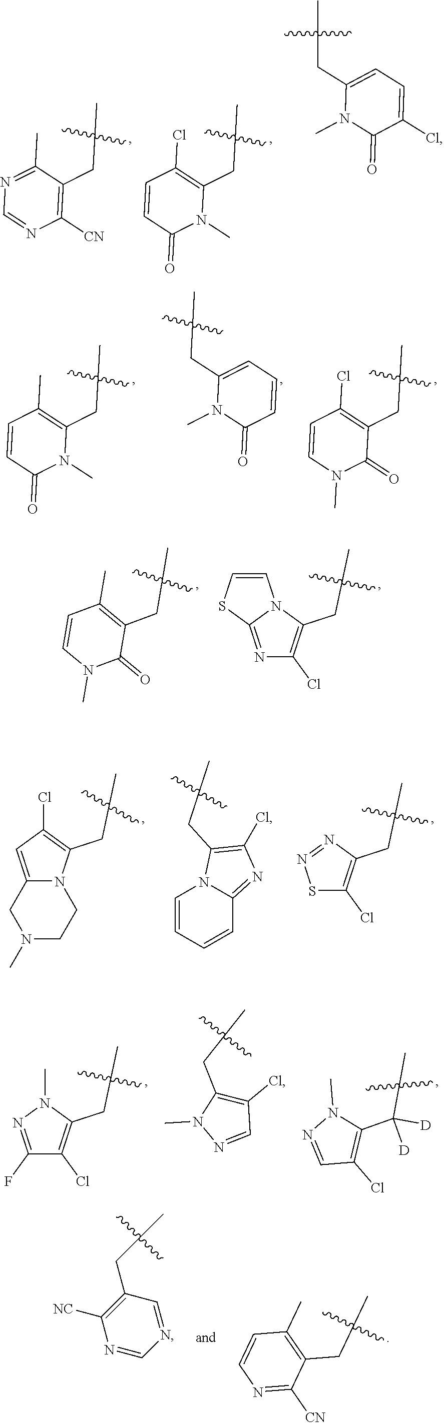

23. The compound, tautomer, solvate, stereoisomer, or pharmaceutically acceptable salt of claim 1, wherein R^{c}, for each occurrence, is independently selected from deuterium, halogen, methyl (which is optionally substituted with OR^{s}), trifluoromethyl, ═O, ═S, OR^{x}, —NHC(═O)R^{x}, —NHC(═O)OR^{x}, NR^{p}R^{q}, —NHC(═O)NR^{p}R^{q}, CN, —C(═O)NR^{p}R^{q}, optionally substituted 5-to 6-membered heteroaryl, and optionally substituted 5- to 6-membered heterocyclyl, wherein:
R^{p}, for each occurrence, is independently selected from H and C₁ to C₂ alkyl,
R^{q}, for each occurrence, is independently selected from H, C₁ to C₂ alkyl, phenyl, 5-to 6-membered heteroaryl, 4- to 7-membered heterocyclyl, and CN, and
R^{s}, for each occurrence, is independently selected from H, C₁ to C₂ alkyl (which is optionally substituted with 1-3 groups selected from halogen and deuterium), phenyl, and 4- to 7-membered heterocyclyl,
wherein:
the 4- to 7-membered heterocyclyl of R^{q} and R^{s}, in each occurrence, is optionally substituted with one group selected from C₁ to C₂ alkyl and —O(C₁ to C₂ alkyl), and
wherein: m is selected from 0, 1, 2, and 3.

24. The compound, tautomer, solvate, stereoisomer, or pharmaceutically acceptable salt of claim 1, wherein R^{c}, for each occurrence, is independently selected from deuterium, F, CH₃, CF₃, OH, ═O, ═S, OCH₃, NH₂, —NHC(═O)CH₃, —NHC(═O)NHCH₃, —NHC(═O)NH₂, —NHC(═O)OCH₃, 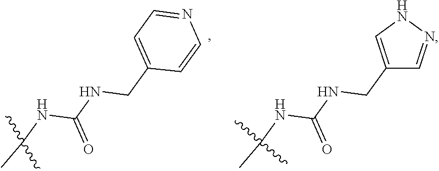 [SMILES: *NC(=O)NCc1ccncc1 ; *NC(=O)NCc1cn[nH]c1]
CN, CH₂OH, —C(═O)NH₂, Cl, —NHCN, OCHF₂, 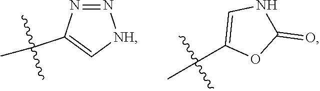 [SMILES: *c1c[nH]nn1 ; *c1c[nH]c(=O)o1]
—SCH₃, OCD₃, and —C(═O)CH₃.

25. The compound, tautomer, solvate, stereoisomer, or pharmaceutically acceptable salt of claim 1, wherein m is zero.

26. The compound, tautomer, solvate, stereoisomer, or pharmaceutically acceptable salt of claim 1, wherein R^{L} is H or deuterium, and R^{y} is H.

27. The compound, tautomer, solvate, stereoisomer, or pharmaceutically acceptable salt of claim 1, wherein: 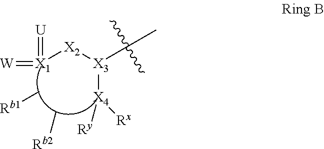
of Formula 1 is: 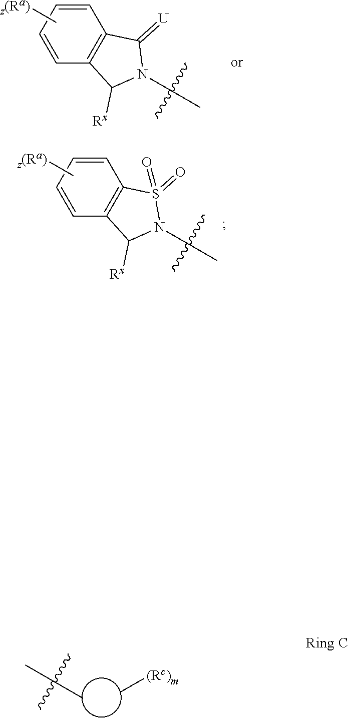
of Formula 1 is selected from: 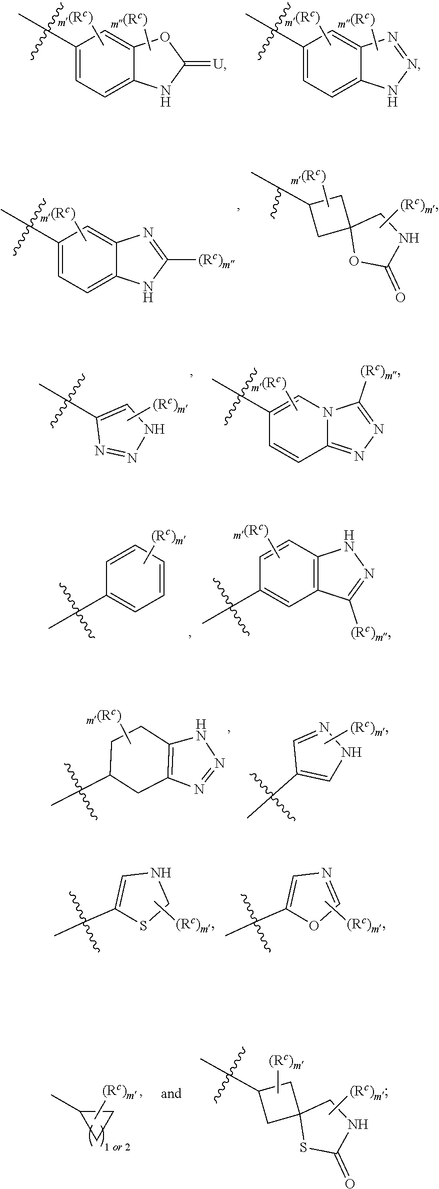
L is selected from —CH₂—, —CD₂—, 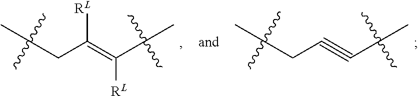
R^{x} is selected from 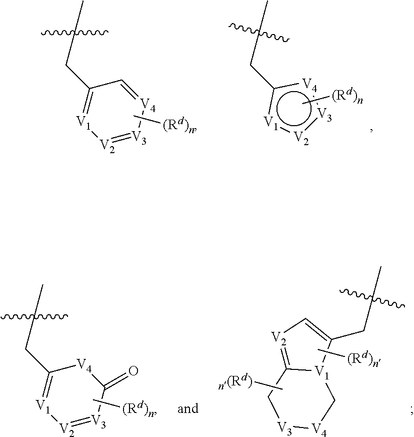
U is O or S;
V₁, V₂, V₃, and V₄ are independently selected from C and N;
R^{L}, for each occurrence, is independently selected from H, deuterium, F, and CH₃;
R^{a}, for each occurrence, is independently selected from CN, halogen, optionally substituted 3- to 4-membered carbocyclyl, optionally substituted 4- to 5-membered heterocyclyl, and optionally substituted O(C₁ to C₃ alkyl);
R^{c}, for each occurrence, is independently selected from deuterium, halogen, OH, C₁ to C₃ alkyl, O(C₁ to C₃ alkyl), and 5- to 6-membered heteroaryl, wherein the O(C₁ to C₃ alkyl) of R^{c} is optionally substituted with 1 to 3 deuteriums;
R^{d}, for each occurrence, is independently selected from halogen, CN, C₁ to C₃ alkyl, and O(C₁ to C₃ alkyl);
z, for each occurrence, is an integer independently selected from 0, 1, 2, and 3;
m′, for each occurrence, is an integer independently selected from 0, 1, and 2;
m″, for each occurrence, is an integer independently selected from 0 and 1;
n, for each occurrence, is an integer independently selected from 0, 1, 2, and 3; and
n′, for each occurrence, is an integer independently selected from 0, 1, and 2.

28. The compound, tautomer, solvate, stereoisomer, or pharmaceutically acceptable salt of claim 1, wherein: 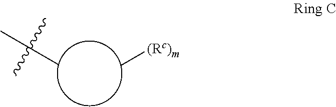
of Formula 1 is selected from: 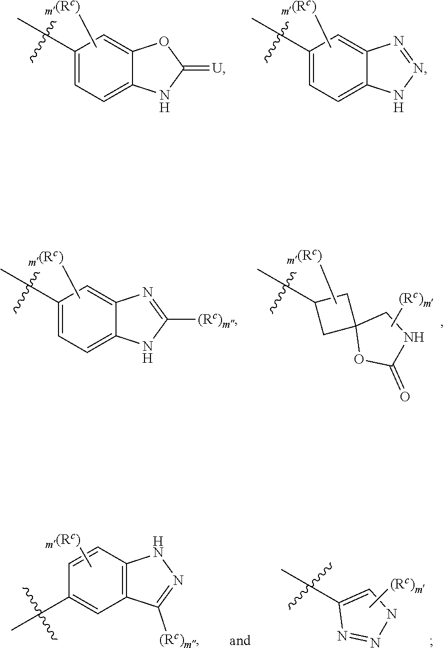
L is selected from —CH₂—, —CD₂—, 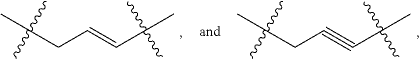 [SMILES: */C=C/C* ; *C#CC*]
R^{x} is selected from 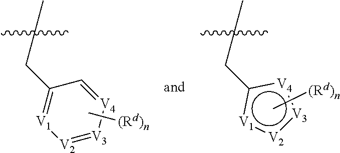
R^{d}, for each occurrence, is independently selected from halogen, CN, C₁ to C₃ alkyl, and O(C₁ to C₃ alkyl);
m′, for each occurrence, is an integer independently selected from 0, 1, and 2;
m″, for each occurrence, is an integer independently selected from 0 and 1; and
n, for each occurrence, is an integer independently selected from 0, 1, 2, and 3.

29. The compound, tautomer, solvate, stereoisomer, or pharmaceutically acceptable salt of claim 1, wherein: 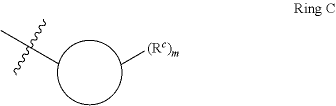
of Formula 1 is selected from: 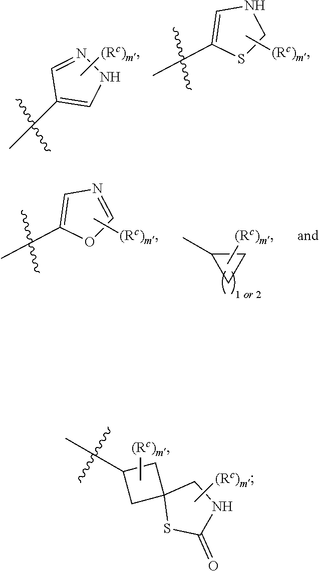
L is selected from —CH₂—, —CD₂—, 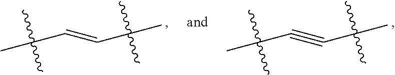
R^{x} is selected from 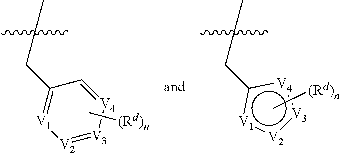
R^{d}, for each occurrence, is independently selected from halogen, CN, C₁ to C₃ alkyl, and O(C₁ to C₃ alkyl);
m′, for each occurrence, is an integer independently selected from 0, 1, and 2; and
n, for each occurrence, is an integer independently selected from 0, 1, 2, and 3.

30. The compound, tautomer, solvate, stereoisomer, or pharmaceutically acceptable salt of claim 1, wherein:
U is O;
V₁, V₂, V₃, and V₄ are independently selected from C and N, wherein no more than two of V₁, V₂, V₃, and V₄ are N;
R^{L} is H or deuterium;
R^{a}, for each occurrence, is independently selected from CN, F, Cl, Br, 3-membered carbocyclyl, 4- to 5-membered heterocyclyl optionally substituted by —N(C₁ to C₂ alkyl)(C₁ to C₂ alkyl), and OCHF₂;
R^{c}, for each occurrence, is independently selected from deuterium, F, OH, CH₃, OCH₃, OCD₃, and 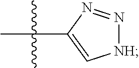 [SMILES: *c1c[nH]nn1]
R^{d}, for each occurrence, is independently selected from F, Cl, Br, CH₃, CH₂CH₃, CN, and OCH₃;
z, for each occurrence, is an integer independently selected from 0, 1, and 2;
m′, for each occurrence, is an integer independently selected from 0, 1, and 2;
n, for each occurrence, is an integer independently selected from 0, 1, 2, and 3; and
n′, for each occurrence, is an integer independently selected from 0, 1, and 2.

31. The compound of claim 1, wherein the compound has a structural formula selected from Formulae 7a-7e: 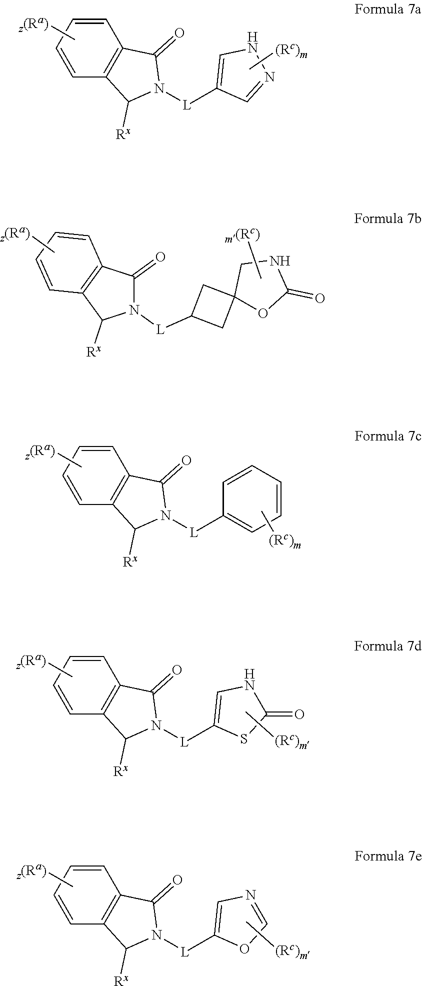
a tautomer thereof, a solvate or stereoisomer of the compound or the tautomer, or a pharmaceutically acceptable salt of the foregoing, wherein L is selected from —CH₂—, —CD₂—, 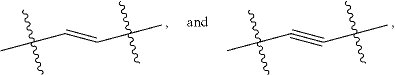
m′ is selected from 0, 1, and 2, and z is selected from 0, 1, and 2.

32. The compound, tautomer, solvate, stereoisomer, or pharmaceutically acceptable salt of claim 31, wherein:
L is selected from —CH₂— and —CD₂—;
R^{a}, for each occurrence, is independently selected from C₁ to C₃ alkyl, halogen, and O(C₁ to C₃ alkyl) optionally substituted with 1 to 3 groups selected from halogen;
R^{c}, for each occurrence, is independently selected from deuterium, halogen, OH, CH₃, OCH₃, and OCD₃;
R^{d}, for each occurrence, is independently selected from halogen, CN, OCH₃, and C₁ to C₄ alkyl;
R^{x} is selected from 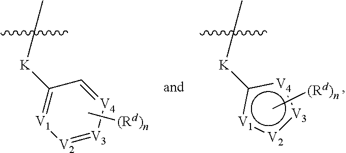
wherein K is selected from —CH₂— and —CD₂—, and
n, for each occurrence, is independently selected from 0, 1, 2, and 3.

33. A compound of the following structural Formula 8: 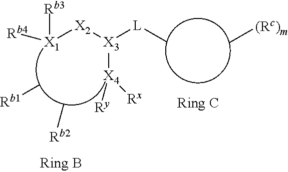
a tautomer thereof, a solvate or stereoisomer of the compound or the tautomer, or a pharmaceutically acceptable salt of the foregoing, wherein:
X₁ is C;
X₂ is C, N, or absent; X₃ is C or N; X₄ is C, N, or absent;
Ring B is a 5- to 6-membered heterocyclic group, wherein Ring B comprises zero or one heteroatom at a position other than the positions of X₁, X₂, X₃, and X₄;
Ring C is phenyl, a 9- to 10-membered aryl, a 5- to 6-membered heteroaryl, a 9-to 10-membered heteroaryl, a 3- to 6-membered carbocyclyl, or a 4- to 12-membered heterocyclic group;
L is selected from 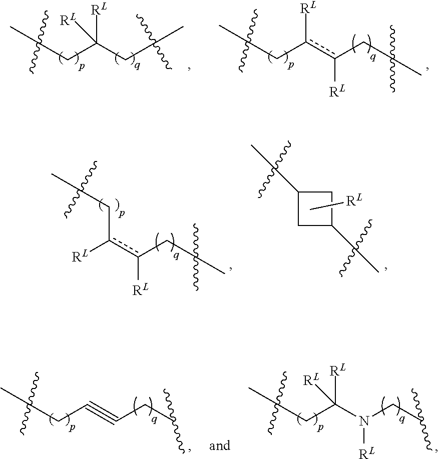
wherein R^{L}, for each occurrence, is independently selected from H, deuterium, halogen, and C₁ to C₃ alkyl optionally substituted with 1-3 groups selected from halogen;
R^{b1} and R^{b2} are attached to two adjacent positions in Ring B and R^{b1} and R^{b2} join to form Ring A, wherein Ring A is a phenyl or 5- to 6-membered heteroaryl group and wherein Ring A is substituted with z groups of R^{a};
R^{b3} and R^{b4} are independently selected from H and C₁ to C₃ alkyl, or R^{b3} and R^{b4} join to form a 3- to 4-membered carbocyclyl,
R^{a}, for each occurrence, is independently selected from halogen, CN, C₁ to C₄ alkyl, —NR^{p}R^{q}, —NR^{p}C(═O)R^{s}, —NR^{p}S(═O)₂R^{q}, OR^{s}, —C(═O)NR^{p}R^{q}, 5- to 6-membered heteroaryl, 3- to 7-membered carbocyclyl, and 3- to 7-membered heterocyclyl,
wherein:
the C₁ to C₄ alkyl of R^{a} is optionally substituted with 1-3 groups selected from —NR^{p}R^{q}, —C(═O)NR^{p}R^{q}, phenyl, 5- to 6-membered heteroaryl, 3- to 7-membered carbocyclyl, 3- to 7-membered heterocyclyl, halogen, and OR^{s},
the 5- to 6-membered heteroaryl, the 3- to 7-membered carbocyclyl, or the 3- to 7-membered heterocyclyl of R^{a} is optionally substituted with 1-2 groups selected from C₁ to C₄ alkyl, —O(C₁ to C₄ alkyl), —N(C₁ to C₄ alkyl)₂, and —NH(C₁ to C₄ alkyl);
R^{x} is selected from H, ═O, CN, C₃ to C₆ carbocyclyl, C₁ to C₄ alkyl, 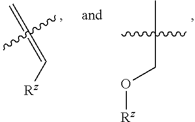
wherein:
the C₁ to C₄ alkyl of R^{x} is optionally substituted with 1-3 groups selected from halogen, deuterium, CN, 5- to 6-membered carbocyclyl, 5- to 9-membered heterocyclyl, 6-membered aryl, 5- to 6-membered heteroaryl, and OH, wherein: the 6-membered aryl or the 5- to 6-membered heteroaryl of the C₁ to C₄ alkyl of R^{x} is optionally substituted with 1-3 groups selected from halogen, OR^{s}, CN, C(═O)NR^{p}R^{q}, NR^{p}R^{q}, C₁-C₆ alkyl (which is optionally substituted with 1-3 groups selected from halogen, O—R^{z}, OR^{s}, and 4- to 6-membered heterocyclyl optionally substituted with C₁ to C₂ alkyl), C₁-C₆ alkenyl, C₁-C₆ alkynyl, 3- to 6-membered carbocyclyl, and 4- to 7-membered heterocyclyl (which is optionally substituted with 1-3 groups selected from optionally substituted C₁ to C₄ alkyl, halogen, and OR^{s}), and
wherein: the 5- to 9-membered heterocyclyl of the C₁ to C₄ alkyl of R^{x} is optionally substituted with 1-2 groups selected from C₁ to C₆ alkyl, ═O, and halogen,
R^{z} is selected from H, 6-membered aryl, and 5- to 6-membered heteroaryl,
wherein: the 6-membered aryl or 5- to 6-membered heteroaryl of R^{z} is optionally substituted with 1-2 groups selected from halogen;
R^{y} is selected from H, C₁ to C₂ alkyl, and absent;
R^{c}, for each occurrence, is independently selected from deuterium, halogen, C₁ to C₄ alkyl, ═O, ═S, OR^{s}, —NHC(═O)R^{s}, —NHC(═O)OR^{s}, NR^{p}R^{q}, —NHC(═O)NR^{p}R^{q}, CN, optionally substituted 5- to 6-membered heteroaryl or optionally substituted 5- to 6-membered heterocyclyl, and —C(═O)NR^{p}R^{q},
wherein:
the C₁ to C₄ alkyl of R^{c} is optionally substituted with 1-3 groups selected from halogen and OR^{s};
wherein:
R^{p}, for each occurrence, is independently selected from H and C₁ to C₆ alkyl, R^{q}, for each occurrence, is independently selected from H, C₁ to C₆ alkyl, phenyl, 5- to 6-membered heteroaryl, 4- to 7-membered heterocyclyl, and CN, or
R^{p} and R^{q} join to form a 3- to 6-membered carbocyclyl;
R^{s}, for each occurrence, is independently selected from H, C₁ to C₆ alkyl (which is optionally substituted with 1-3 groups selected from deuterium, halogen, O(C₁ to C₄ alkyl), and NR^{p}R^{q}), phenyl, and 4- to 7-membered heterocyclyl;
wherein:
the 4- to 7-membered heterocyclyl of R^{a} and R^{s}, in each occurrence, is optionally substituted with 1-2 groups selected from C₁ to C₄ alkyl and —O(C₁ to C₄ alkyl); and
wherein:
m is an integer selected from 0, 1, 2, and 3;
p is an integer selected from 0, 1, 2, and 3;
q is an integer selected from 0, 1, 2, and 3;
z is an integer selected from 0, 1, 2, 3, and 4; and
the sum of p and q is an integer equal to or less than 5.

34. The compound of claim 33, wherein the compound has a structural formula selected from Formulae 9a-9h: 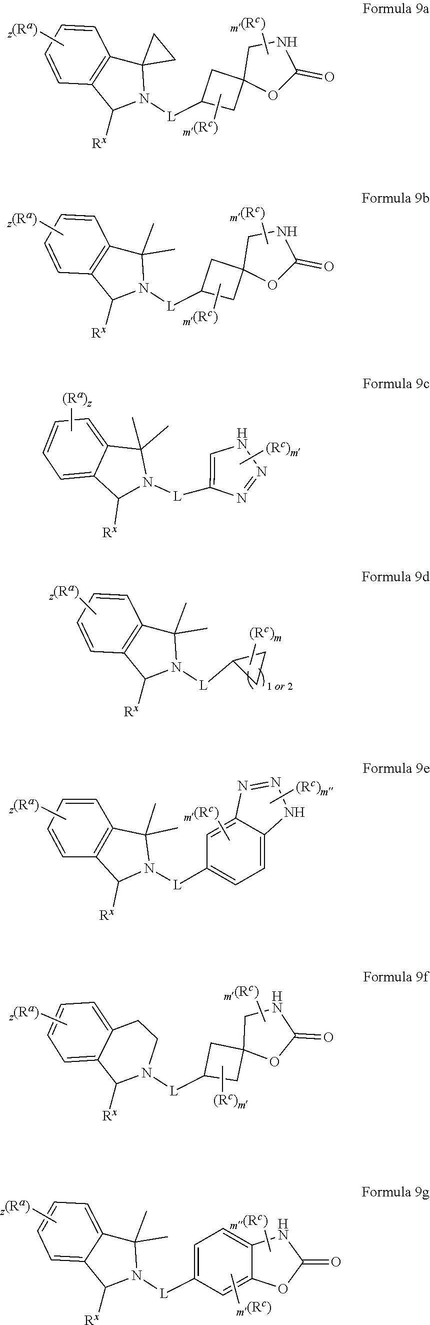 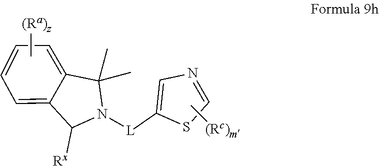
a tautomer thereof, a solvate or stereoisomer of the compound or the tautomer, or a pharmaceutically acceptable salt of the foregoing, wherein:
L is selected from —CH₂—, —CD₂—, 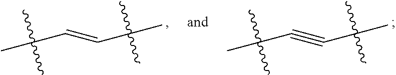
m′, for each occurrence, is independently selected from 0, 1, and 2, m″, for each occurrence, is independently selected from 0, and 1, z, for each occurrence, is independently selected from 0, 1, and 2.

35. The compound, tautomer, solvate, stereoisomer, or pharmaceutically acceptable salt of claim 34, wherein:
L is selected from —CH₂—, —CD₂—, and 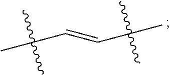 [SMILES: */C=C/*]
R^{a} is selected from C₁ to C₃ alkyl, CN, and halogen;
R^{x} is selected from 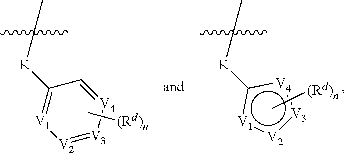
wherein K is selected from —CH₂— and —CD₂—;
R^{c}, for each occurrence, is independently selected from deuterium, C₁ to C₃ alkyl, halogen, 5- to 6-membered heteroaryl, OH, and O(C₁ to C₃ alkyl) optionally substituted with 1 to 3 groups selected from halogen and deuterium,
R^{d}, for each occurrence, is independently selected from halogen, CN, OCH₃, —C(═O)NH₂, and C₁ to C₄ alkyl; and
n, for each occurrence, is independently selected from 0, 1, 2 and 3.

36. The compound, tautomer, solvate, stereoisomer, or pharmaceutically acceptable salt of claim 35, wherein:
R^{a} is selected from F and Cl,
R^{x} is selected from: 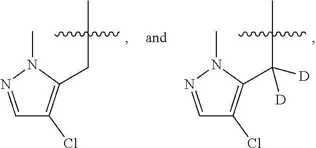
R^{c}, for each occurrence, is independently selected from: deuterium, F, OH, OCH₃, OCD₃, and C(═O)NH₂;
m, for each occurrence, is independently 0, 1, or 2; m′, for each occurrence, is independently 0, 1 or 2; and
z, for each occurrence, is independently 0, 1, or 2.

37. A compound selected from Compounds 1 to 645 in Table 1, a tautomer thereof, a solvate or stereoisomer of the compound or the tautomer, or a pharmaceutically acceptable salt of the foregoing.

38. A pharmaceutical composition comprising a compound according to claim 1, a tautomer thereof, a solvate or stereoisomer of the compound or the tautomer, or a pharmaceutically acceptable salt of the foregoing and at least one pharmaceutically acceptable carrier.

39. A method of treating a disease or condition, comprising administering to a subject in need thereof, a therapeutically effective amount of a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier, wherein the disease or condition is selected from cardiac diseases, renal diseases, hyperproliferative diseases or conditions, cancer, chemotherapy induced tissue damage, renal diseases, metabolic diseases, muscular diseases, neurological diseases and injuries, inflammatory diseases or conditions, mitochondrial diseases, ocular diseases, diseases caused by impaired stem cell function, DNA damages, primary mitochondrial disorders, obesity, atherosclerosis, insulin resistance, diabetes, complications associated with diabetes, Alzheimer's disease, Huntington's disease, Parkinson's disease, amyotrophic lateral sclerosis, depression, Down syndrome, neonatal nerve injury, aging, axonal degeneration, carpal tunnel syndrome, Guillain-Barre syndrome, nerve damage, polio (poliomyelitis), and spinal cord injury.

40. A method of treating a disease or condition responsive to NAMPT activation, comprising administering to a subject in need thereof, a therapeutically effective amount of a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier.

41. A method of modulating NAMPT, comprising contacting a subject in need thereof with a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier.

42. A method of increasing NAD⁺ level, comprising contacting a subject in need thereof with a compound according to claim 1, a tautomer thereof, a solvate or stereoisomer of the compound or the tautomer, or a pharmaceutically acceptable salt of the foregoing, or a pharmaceutical composition comprising the compound of claim 1, a tautomer thereof, a solvate or stereoisomer of the compound or the tautomer, or a pharmaceutically acceptable salt of the foregoing and at least one pharmaceutically acceptable carrier.
